(19) United States
(12) Patent Application Publication
(10) Pub. No.: US 2025/0388605 A1
(43) Pub. Date: Dec. 25, 2025

(54) HETEROBIFUNCTIONAL COMPOUNDS AND METHODS OF TREATING DISEASE

(71) Applicant: Halda Therapeutics OpCo, Inc., New Haven, CT (US)

(72) Inventors: Kyle J. Eastman, Killingworth, CT (US); Katherine J. Kayser-Bricker, Branford, CT (US); Michael Barrington Martin, Manchester, CT (US); James John Mousseau, Salem, CT (US); Matthew Alexander Perry, Uncasville, CT (US); David E. Puleo, Fairfield, CT (US); Kanak Shail Raina, Branford, CT (US); Nilesh K Zaware, Hamden, CT (US); Taavi Neklesa, Orange, CT (US); Samuel W. Gerritz, Guilford, CT (US); Todd Bosanac, Burlington, CT (US)

(21) Appl. No.: 19/108,548

(22) PCT Filed: Sep. 8, 2023
(86) PCT No.: PCT/US2023/073698

§ 371 Date: Mar. 4, 2025

Related U.S. Application Data
(60) Provisional application No. 63/404,575, filed on Sep. 8, 2022.
(60) Provisional application No. 63/506,790, filed on Jun. 7, 2023.

(51) Int. Cl. C07D 519/00; A61K 31/444; A61K 31/4545; A61K 31/497; A61K 31/4995; A61K 31/501; A61K 31/506; A61K 31/519; A61K 31/5377; A61K 31/5386; A61K 31/55; A61P 35/00; C07D 471/04

(52) U.S. Cl. CPC C07D 519/00; A61K 31/444; A61K 31/4545; A61K 31/497; A61K 31/4995; A61K 31/501; A61K 31/506; A61K 31/519; A61K 31/5377; A61K 31/5386; A61K 31/55; A61P 35/00; C07D 471/04

(57) ABSTRACT

The invention provides heterobifunctional compounds which may bind to both an androgen receptor and BRD4 (bromodomain-containing protein 4). Also provided are pharmaceutical compositions comprising the same and their use in treating disease, such as cancer.

### CROSS-REFERENCE TO RELATED APPLICATIONS

This application claims the benefit of and priority to U.S. Provisional Patent Application Ser. No. 63/404,575, filed Sep. 8, 2022, and U.S. Provisional Patent Application Ser. No. 63/506,790, filed Jun. 7, 2023; the contents of each of which are hereby incorporated by reference in their entirety.

### FIELD OF THE INVENTION

The invention provides heterobifunctional compounds, pharmaceutical compositions, and their use in treating disease, such as cancer.

### BACKGROUND

Cancer continues to be a significant health problem despite the substantial research efforts and scientific advances reported in the literature for treating this disease. Solid tumors, including prostate cancer, breast cancer, and lung cancer remain highly prevalent among the world population. The incidence of prostate cancer increases with age, and with increasing longevity of human subjects, there continues to be a corresponding rise in the number of patients suffering from prostate cancer. Breast cancer is one of the most common cancers among women and is a leading cause of death for women between ages 50-55. Lung cancer is a leading cause of death among cancer patients, where over 85% of lung cancers are non-small cell lung cancer (NSCLC). Many lung cancers are attributed to tobacco smoking. Current treatment options for these cancers are not effective for all patients and/or can have substantial adverse side effects.

New therapies are needed to address this unmet need in cancer therapy. In particular, new therapies are needed that achieve an anti-cancer effect through a different mechanism than commonly available therapies. Exemplary mechanisms for common anti-cancer therapies include (a) alkylation of DNA which limits ability of the cell to reproduce, (b) topoisomerase inhibition, in which the therapeutic agent inhibits the activity of a topoisomerases thereby limiting separation of strands of DNA, and (c) mitotic inhibition, where the therapeutic agent reduces ability of the cell to divide. New therapies that achieve an anti-cancer effect through a different mechanism present an opportunity to treat cancers more effectively and/or to treat cancers that have become resistant to currently available medicines.

The present invention addresses the foregoing needs and provides other related advantages.

### SUMMARY

The invention provides heterobifunctional compounds, pharmaceutical compositions, and their use in treating disease, such as cancer. In particular, one aspect of the invention provides a collection of heterobifunctional compounds, such as a compound represented by Formula I:

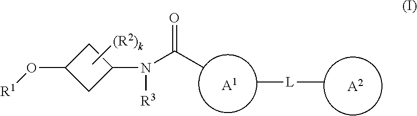

or a pharmaceutically acceptable salt thereof, where the variables are as defined in the detailed description. Further description of additional collections of heterobifunctional compounds are described in the detailed description. The compounds may be part of a pharmaceutical composition comprising a pharmaceutically acceptable carrier.

Another aspect of the invention provides a collection of heterobifunctional compounds, such as a compound represented by Formula I-1:

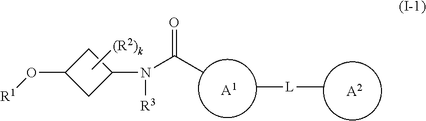

or a pharmaceutically acceptable salt thereof, where the variables are as defined in the detailed description. Further description of additional collections of heterobifunctional compounds are described in the detailed description. The compounds may be part of a pharmaceutical composition comprising a pharmaceutically acceptable carrier.

Another aspect of the invention provides a collection of heterobifunctional compounds, such as a compound represented by Formula I-1*:

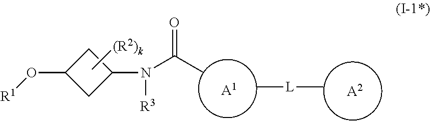

or a pharmaceutically acceptable salt thereof, where the variables are as defined in the detailed description. Further description of additional collections of heterobifunctional compounds are described in the detailed description. The compounds may be part of a pharmaceutical composition comprising a pharmaceutically acceptable carrier.

Another aspect of the invention provides a collection of heterobifunctional compounds, such as a compound represented by Formula II:

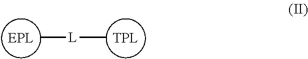

or a pharmaceutically acceptable salt thereof, where the variables are as defined in the detailed description. Further description of additional collections of heterobifunctional compounds are described in the detailed description. The compounds may be part of a pharmaceutical composition comprising a pharmaceutically acceptable carrier.

Another aspect of the invention provides a method of treating cancer. The method comprises administering to a patient in need thereof a therapeutically effective amount of a compound described herein, such as a compound of Formula I or II, to treat the cancer.

Another aspect of the invention provides a method of causing death of a cancer cell. The method comprises contacting a cancer cell with an effective amount of a compound described herein, such as a compound of Formula I or II, to cause death of the cancer cell.

### DETAILED DESCRIPTION

The invention provides heterobifunctional compounds, pharmaceutical compositions, and their use in treating disease, such as cancer. The practice of the present invention employs, unless otherwise indicated, conventional techniques of organic chemistry, pharmacology, molecular biology (including recombinant techniques), cell biology, biochemistry, and immunology. Such techniques are explained in the literature, such as in “Comprehensive Organic Synthesis” (B. M. Trost & I. Fleming, eds., 1991-1992); “Handbook of experimental immunology” (D. M. Weir & C. C. Blackwell, eds.); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety.

Various aspects of the invention are set forth below in sections; however, aspects of the invention described in one particular section are not to be limited to any particular section. Further, when a variable is not accompanied by a definition, the previous definition of the variable controls.

### Definitions

Compounds of the present invention include those described generally herein, and are further illustrated by the classes, subclasses, and species disclosed herein. As used herein, the following definitions shall apply unless otherwise indicated. These definitions apply regardless of whether a term is used by itself or in combination with other terms, unless otherwise indicated. Hence, the definition of “alkyl” applies to “alkyl” as well as the “alkyl” portions of “—O-alkyl” etc. For purposes of this invention, the chemical elements are identified in accordance with the Periodic Table of the Elements, CAS version, Handbook of Chemistry and Physics, ₇₅^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e., carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

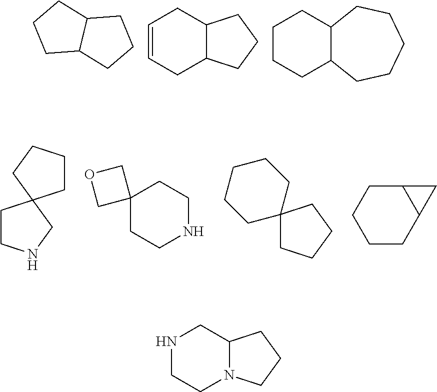

Exemplary bridged bicyclics include:

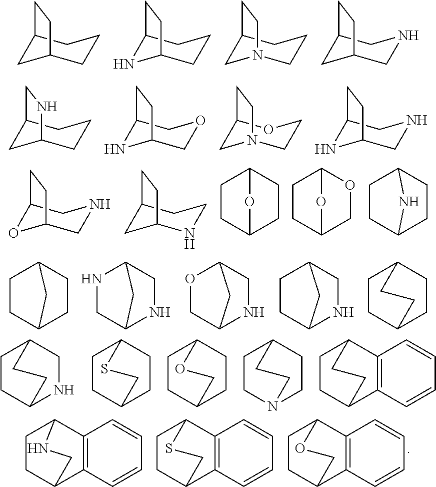

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “—(C₀ alkylene)-” refers to a bond. Accordingly, the term “—(C₀₋₃ alkylene)-” encompasses a bond (i.e., C₀) and a —(C₁₋₃ alkylene)- group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “haloaryl” refers to an aryl group that is substituted with at least one halogen. Exemplary haloaryl groups include chlorophenyl (e.g., 3-chlorophenyl, 4-chlorophenyl), fluorophenyl, and the like. The term “phenylene” refers to a bivalent phenyl group.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted. The term “haloheteroaryl” refers to a heteroaryl group that is substituted with at least one halogen. Exemplary haloheteroaryl groups include chloropyridine, fluoropyridine, chloropyrazole, fluoropyrazole, and the like. The term “heteroarylene” refers to a bivalent heteroaryl group. Similarly, the terms “pyrazolylene”, “imidazolylene”, and “pyrrolylene”, respectively refer to bivalent pyrazolyl, imidazolyl, and pyrrolyl groups. Similarly, the terms “pyridazinylene,” “pyrimidinylene,” “pyrazinylene,” and “pyridinylene,” respectively refer to bivalent pyridazinyl, pyrimidinyl, pyrazinyl, and pyridinyl groups.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted. The term “heterocyclylene” refers to a bivalent heterocyclyl group. The terms “piperidinylene,” “piperazinylene,” and “azetidinylene”, respectively refer to bivalent piperidinyl, piperazinyl, and azetidinyl groups.

As used herein, the term “heterocycloalkyl” refers to a saturated heterocyclyl. The term “heterocycloalkylene” refers to a bivalent heterocycloalkyl group.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R°; —(CH₂)₀₋₄OR°; —O(CH₂)₀₋₄R°, —O—(CH₂)₀₋₄C(O)OR°; —(CH₂)₀₋₄CH(OR°)₂; —(CH₂)₀₋₄SR°; —(CH₂)₀₋₄Ph, which may be substituted with R°; —(CH₂)₀₋₄O(CH₂)₀₋₁Ph which may be substituted with R°; —CH═CHPh, which may be substituted with R°; —(CH₂)₀₋₄O(CH₂)₀₋₁-pyridyl which may be substituted with R°; —NO₂; —CN; —N₃; —(CH₂)₀₋₄N(R°)₂; —(CH₂)₀₋₄N(R°)C(O)R°; —N(R°)C(S)R°; —(CH₂)₀₋₄N(R°)C(O)NR°₂; —N(R°)C(S)NR°₂; —(CH₂)₀₋₄N(R°)C(O)OR°; —N(R°)N(R°)C(O)R°; —N(R°)N(R°)C(O)NR°₂, —N(R°)N(R°)C(O)OR°; —(CH₂)₀₋₄C(O)R°; —C(S)R°; —(CH₂)₀₋₄C(O)OR°; —(CH₂)₀₋₄C(O)SR°; —(CH₂)₀₋₄C(O)OSiR°₃; —(CH₂)₀₋₄OC(O)R°; —OC(O)(CH₂)₀₋₄SR—, SC(S)SR°; —(CH₂)₀₋₄SC(O)R°; —(CH₂)₀₋₄C(O)NR°₂; —C(S)NR°₂; —C(S)SR°; —SC(S)SR°, —(CH₂)₀₋₄OC(O)NR°₂; —C(O)N(OR°)R°; —C(O)C(O)R°; —C(O)CH₂C(O)R°; —C(NOR°)R°; —(CH₂)₀₋₄SSR°; —(CH₂)₀₋₄S(O)₂R°; —(CH₂)₀₋₄S(O)₂OR°; —(CH₂)₀₋₄OS(O)₂R°; —S(O)₂NR°₂; —S(O)(NR°)R°; —S(O)₂N═C(NR°₂)₂; —(CH₂)₀₋₄S(O)R°; —N(R°)S(O)₂NR°₂; —N(R°)S(O)₂R°; —N(OR°)R°; —C(NH)NR°₂; —P(O)₂R°; —P(O)R°₂; —OP(O)R°₂; —OP(O)(OR°)₂; SiR°₃; —(C₁₋₄ straight or branched alkylene)O—N(R°)₂; or —(C₁₋₄ straight or branched alkylene)C(O)O—N(R°)₂.

Each R° is independently hydrogen, C₁₋₄ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R°, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R° selected from ═O and =S; or each R° is optionally substituted with a monovalent substituent independently selected from halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•}.

Each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens; or wherein an optional substituent on a saturated carbon is a divalent substituent independently selected from ═O, ═S, ═NNR⁺ ₂, ═NNHC(O)R^{•}, ═NNHC(O)OR^{•}, ═NNHS(O)₂R^{•}, ═NR⁺, ═NOR^{•}, —O(C(R^{•}₂))₋₃O—, or —S(C(R^{•}2))₂₋₃S—, or a divalent substituent bound to vicinal substitutable carbons of an “optionally substituted” group is —O(CR^{•}₂)₂₋₃₀—, wherein each independent occurrence of R^{•} is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R^{•} is C₁₋₆ aliphatic, R^{•} is optionally substituted with halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens.

An optional substituent on a substitutable nitrogen is independently —R^{\}, —NR^{\}₂, —C(O)R^{\}, —C(O)OR^{\}, —C(O)C(O)R^{\}, —C(O)CH₂C(O)R^{\}, —S(O)₂R^{\}, —S(O)₂NR^{\}₂, —C(S)NR^{\}2, —C(NH)NR^{\}₂, or —N(R^{\})S(O)₂R^{\}; wherein each R^{\} is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of R^{\}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R^{\} is C₁₋₆ aliphatic, R^{\} is optionally substituted with halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}2, or —NO₂, wherein each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. The invention includes compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as a atropisomer (e.g., substituted biaryls), all forms of such atropisomer are considered part of this invention.

Chemical names, common names, and chemical structures may be used interchangeably to describe the same structure. If a chemical compound is referred to using both a chemical structure and a chemical name, and an ambiguity exists between the structure and the name, the structure predominates. It should also be noted that any carbon as well as heteroatom with unsatisfied valences in the text, schemes, examples and tables herein is assumed to have the sufficient number of hydrogen atom(s) to satisfy the valences.

The terms “a” and “an” as used herein mean “one or more” and include the plural unless the context is inappropriate.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl. The term “cycloalkylene” refers to a bivalent cycloalkyl group.

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like. The term “chloroalkyl” refers to an alkyl group that is substituted with at least one chloro. The term “bromoalkyl” refers to an alkyl group that is substituted with at least one bromo. The term “haloalkylene” refers to a bivalent haloalkyl group.

The term “hydroxyalkyl” refers to an alkyl group that is substituted with at least one hydroxyl. Exemplary hydroxyalkyl groups include —CH₂CH₂OH, —C(H)(OH)CH₃, —CH₂C(H)(OH)CH₂CH₂OH, and the like.

The term “heteroalkyl” refers to an alkyl group in which one or more carbon atoms has been replaced by a heteroatom (e.g., N, O, or S). Exemplary heteroalkyl groups include —OCH₃, —CH₂OCH₃, —CH₂CH₂N(CH₃)₂, and —CH₂CH₂OH. The heteroalkyl group may contain, for example, from 2-4, 2-6, or 2-8 atoms selected from the group consisting of carbon and a heteroatom (e.g., N, O, or S). The phrase 3-8 membered heteroalkyl refers to a heteroalkyl group having from 3 to 8 atoms selected from the group consisting of carbon and a heteroatom. The term “heteroalkylene” refers to a bivalent heteroalkyl group.

The terms “alkenyl” and “alkynyl” are art-recognized and refer to unsaturated aliphatic groups analogous in length and possible substitution to the alkyls described above, but that contain at least one double or triple bond respectively. The term “haloalkenyl” refers to an alkenyl group that is substituted with at least one halogen. The term “fluoroalkenyl” refers to an alkenyl group that is substituted with at least one fluoro. The term “nitroalkenyl” refers to an alkenyl group that is substituted with at least one nitro.

The term “carbocyclylene” refers to a bivalent cycloaliphatic group.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. The term “haloalkoxyl” refers to an alkoxyl group that is substituted with at least one halogen. Exemplary haloalkoxyl groups include —OCH₂F, —OCHF₂, —OCF₃, —OCH₂CF₃, —OCF₂CF₃, and the like.

The term “oxo” is art-recognized and refers to a “═O” substituent. For example, a cyclopentane susbsituted with an oxo group is cyclopentanone.

The term “amino” is art-recognized and refers to both unsubstituted and substituted amines, e.g., a moiety that may be represented by the general formulas:

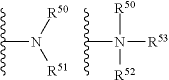

wherein R⁵⁰, R⁵¹, R⁵² and R⁵³ each independently represent a hydrogen, an alkyl, an alkenyl, —(CH₂)ₘ—R⁶¹, or R⁵⁰ and R⁵¹, taken together with the N atom to which they are attached complete a heterocycle having from 4 to 8 atoms in the ring structure; R⁶¹ represents an aryl, a 3-7 membered cycloalkyl, a 4-7 membered cycloalkenyl, 5-10 membered heteroaryl, or 3-10 membered heterocyclyl; and m is zero or an integer in the range of 1 to 8.

The term “amido” is art-recognized and refers to both unsubstituted and substituted amides, e.g., a moiety that may be represented by the general formulas:

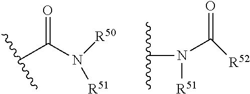

wherein R⁵⁰ and R⁵¹ each independently represent a hydrogen, an alkyl, an alkenyl, —(CH₂)ₘ—R⁶¹, or R⁵⁰ and R⁵¹, taken together with the N atom to which they are attached complete a heterocycle having from 4 to 8 atoms in the ring structure; R⁶¹ represents an aryl, a 3-7 membered cycloalkyl, a 4-7 membered cycloalkenyl, 5-10 membered heteroaryl, or 3-10 membered heterocyclyl; and m is zero or an integer in the range of 1 to 8; and R⁵² is an alkyl, an alkenyl, or —(CH₂)ₘ—R⁶¹.

The symbol “ [symbol] ” indicates a point of attachment.

When any substituent or variable occurs more than one time in any constituent or the compound of the invention, its definition on each occurrence is independent of its definition at every other occurrence, unless otherwise indicated.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

As used herein, the terms “subject” and “patient” are used interchangeable and refer to organisms to be treated by the methods of the present invention. Such organisms preferably include, but are not limited to, mammals (e.g., murines, simians, equines, bovines, porcines, canines, felines, and the like), and most preferably includes humans.

The term “IC₅₀” is art-recognized and refers to the concentration of a compound that is required to achieve 50% inhibition of the target.

As used herein, the term “effective amount” refers to the amount of a compound sufficient to effect beneficial or desired results (e.g., a therapeutic, ameliorative, inhibitory or preventative result). An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

In addition, when a compound of the invention contains both a basic moiety (such as, but not limited to, a pyridine or imidazole) and an acidic moiety (such as, but not limited to, a carboxylic acid) zwitterions (“inner salts”) may be formed. Such acidic and basic salts used within the scope of the invention are pharmaceutically acceptable (i.e., non-toxic, physiologically acceptable) salts. Such salts of the compounds of the invention may be formed, for example, by reacting a compound of the invention with an amount of acid or base, such as an equivalent amount, in a medium such as one in which the salt precipitates or in an aqueous medium followed by lyophilization.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified.

### I. Heterobifunctional Compounds

One aspect of the invention provides heterobifunctional compounds. The compounds may be used in the pharmaceutical compositions and therapeutic methods described herein. Exemplary compounds are described in the following sections, along with exemplary procedures for making the compounds. Without being bound by theory, the compounds can facilitate therapeutic effects by binding to both an androgen receptor and BRD4 (bromodomain-containing protein 4).

### Part A: Compound of Formula T

One aspect of the invention provides a compound represented by Formula I:

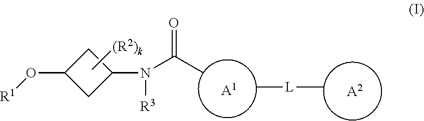

or a pharmaceutically acceptable salt thereof, wherein:
R¹ is phenyl substituted by cyano, halogen, and m occurrences of R⁴;
R² represents independently for each occurrence C₁₋₄ alkyl;
R³ is hydrogen or C₁₋₄ alkyl;
R⁴ is C₁₋₄ alkyl;
R⁵ represents independently for each occurrence C₁₋₄ alkyl or halogen;
A¹ is a pyridazinylene, pyrimidinylene, pyrazinylene, pyridinylene, or phenylene, each of which is substituted with n occurrences of R⁵;
A² is one of the following:

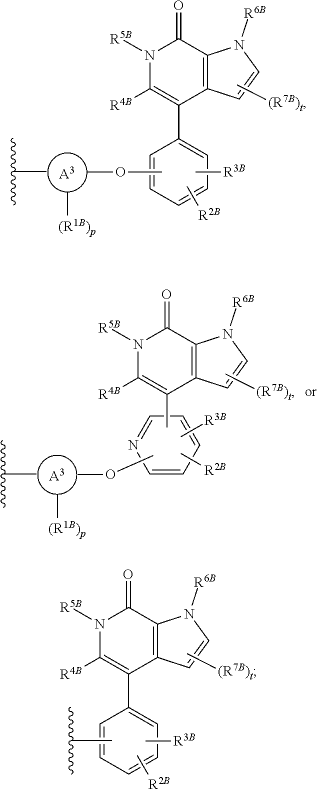

A³ is phenylene, a 3-10 membered saturated monocyclic, bicyclic or spirocyclic carbocycylene, or C₁₋₆ alkylene;
R^{1B} represents independently for each occurrence halo, C₁₋₄ alkyl, or C₁₋₄ haloalkyl;
R^{2B} is hydrogen, halo, C₁₋₄ alkyl, or C₁₋₄ haloalkyl;
R^{3B} is —N(R^{8B})SO₂R^{9B}, —SO₂N(R^{8B})₂, —SO₂R^{9B}, —(C₁₋₆ alkylene)-SO₂R^{9B}, —(C₀₋₆ alkylene)-N(R^{8B})C(O)R^{9B}, —(C₀₋₆ alkylene)-C(O)N(R^{8B})₂, —N(R^{8B})₂, —NO₂, C₁₋₆ hydroxyalkyl, hydrogen, or a 4-7 membered saturated carbocyclic ring in which one CH₂ is replaced with SO₂;
R^{4B} is hydrogen, halo, or C₁₋₄ alkyl;
R^{5B} is C₁₋₄ alkyl or C₃₋₄ cycloalkyl;
R^{6B} is hydrogen, C₁₋₄ alkyl, or C₃₋₄ cycloalkyl;
R^{7B} represents independently for each occurrence C₁₋₄ alkyl or C₃₋₄ cycloalkyl;
R^{8B} represents independently for each occurrence hydrogen or C₁₋₄ alkyl; or two occurrences of R⁸ are taken together with the nitrogen atom to which they are attached to form a 3-7 membered heterocyclic ring containing 1 or 2 heteroatoms independently selected from nitrogen and oxygen; or R^{8B} and R^{9B} are taken together with their intervening atoms to a form a 5-7 membered ring containing 1, 2, or 3 heteroatoms independently selected from nitrogen, oxygen, and sulfur;
R^{9B} is C₁₋₆ alkyl, C₁₋₆ haloalkyl, —(C₁₋₆ alkylene)-(C₃₋₆ cycloalkyl), or C₃₋₆ cycloalkyl; and
L is a linker;
k is 1, 2, 3, or 4; and
m, n, p and t are independently 0, 1, or 2.

The definitions of variables in Formula I above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I.

As define generally above, R¹ is phenyl substituted by cyano, halogen, and m occurrences of R⁴. In certain embodiments, R¹ is phenyl substituted by cyano, halogen, and 0 occurrences of R⁴. In certain embodiments, R¹ is phenyl substituted by cyano, halogen, and 1 occurrence of R⁴. In certain embodiments, R¹ is phenyl substituted by cyano, halogen, and 2 occurrences of R⁴. In certain embodiments, R¹ is phenyl substituted by cyano, Cl, and 0 occurrences of R⁴. In certain embodiments, R¹ is

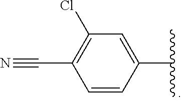 [SMILES: *c1ccc(C#N)c(Cl)c1]

In certain embodiments, R¹ is selected from the groups depicted in the compounds in Table 1 below.

As defined generally above, R² represents independently for each occurrence C₁₋₄ alkyl. In certain embodiments, R² is C₁₋₂ alkyl. In certain embodiments, R² is methyl. In certain embodiments, R² is selected from the groups depicted in the compounds in Table 1 below.

As defined generally above, R³ is hydrogen or C₁₋₄ alkyl. In certain embodiments, R³ is hydrogen. In certain embodiments, R³ is C₁₋₄ alkyl. In certain embodiments, R³ is selected from the groups depicted in the compounds in Table 1 below.

As defined generally above, A¹ is a pyridazinylene, pyrimidinylene, pyrazinylene, pyridinylene, or phenylene, each of which is substituted with n occurrences of R⁵. In certain embodiments, A¹ is pyridazinylene substituted with n occurrences of R⁵. In certain embodiments, A¹ is

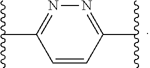 [SMILES: *c1ccc(*)nn1]

In certain embodiments, A¹ is pyrimidinylene substituted with n occurrences of R⁵. In certain embodiments, A¹ is

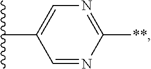

where ** is the point of attachment to L. In certain embodiments, A¹ is

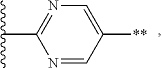

where ** is the point of attachment to L. In certain embodiments, A¹ is pyrazinylene substituted with n occurrences of R⁵. In certain embodiments, A¹ is

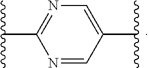 [SMILES: *c1cnc(*)nc1]

In certain embodiments, A¹ is pyridinylene substituted with n occurrences of R⁵. In certain embodiments, A¹ is

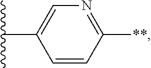

where ** is the point of attachment to L. In certain embodiments, A¹ is phenylene substituted with n occurrences of R⁵. In certain embodiments, A¹ is

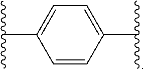 [SMILES: *c1ccc(*)cc1]

In certain embodiments, A¹ is a pyridazinylene substituted with 0 occurrences of R⁵. In certain embodiments, A¹ is a pyrimidinylene substituted with 0 occurrences of R⁵. In certain embodiments, A¹ is a pyrazinylene substituted with 0 occurrences of R⁵. In certain embodiments, A¹ is a pyridinylene, substituted with 0 occurrences of R⁵. In certain embodiments, A¹ is a phenylene substituted with 0 occurrences of R⁵. In certain embodiments, A¹ is selected from the groups depicted in the compounds in Table 1 below.

As defined generally above, A² is

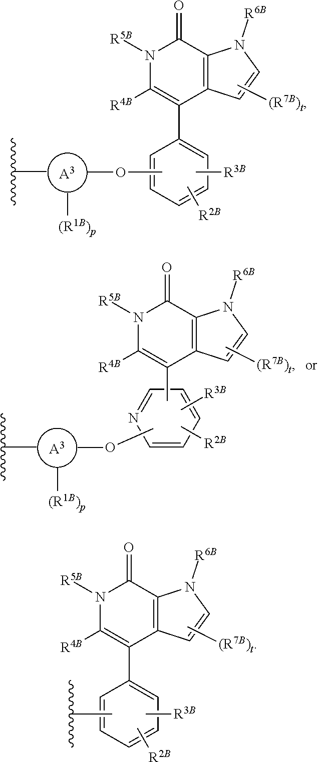

In certain embodiments, A² is

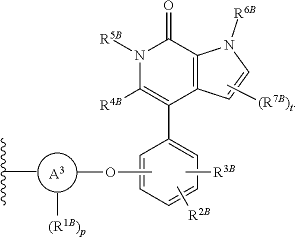

In certain embodiments, A² is

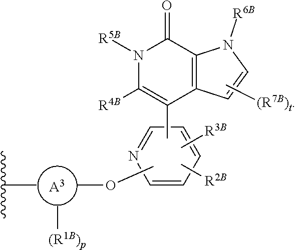

In certain embodiments, A² is

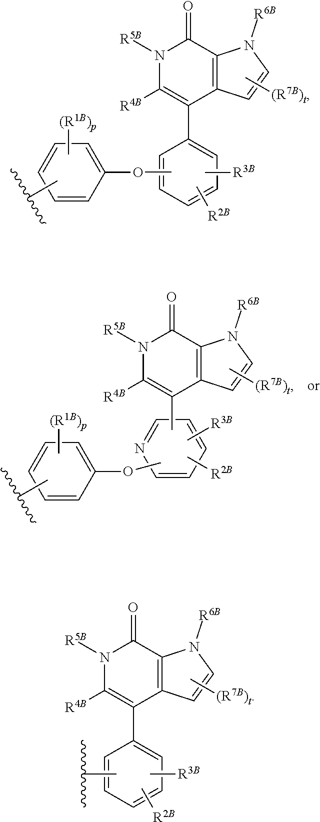

In certain embodiments, A² is

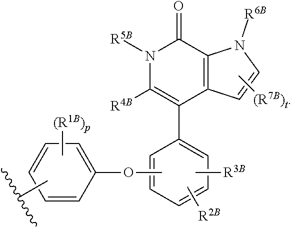

In certain embodiments, A² is

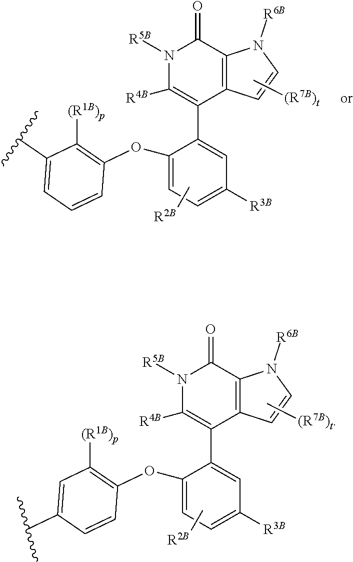

In certain embodiments, A² is

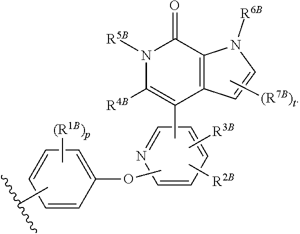

In certain embodiments, A² is

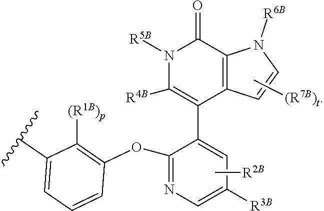

In certain embodiments, A² is

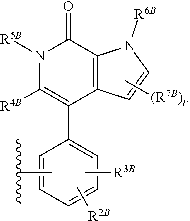

In certain embodiments, A² is

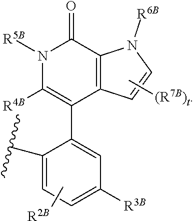

In certain
embodiments, A² is

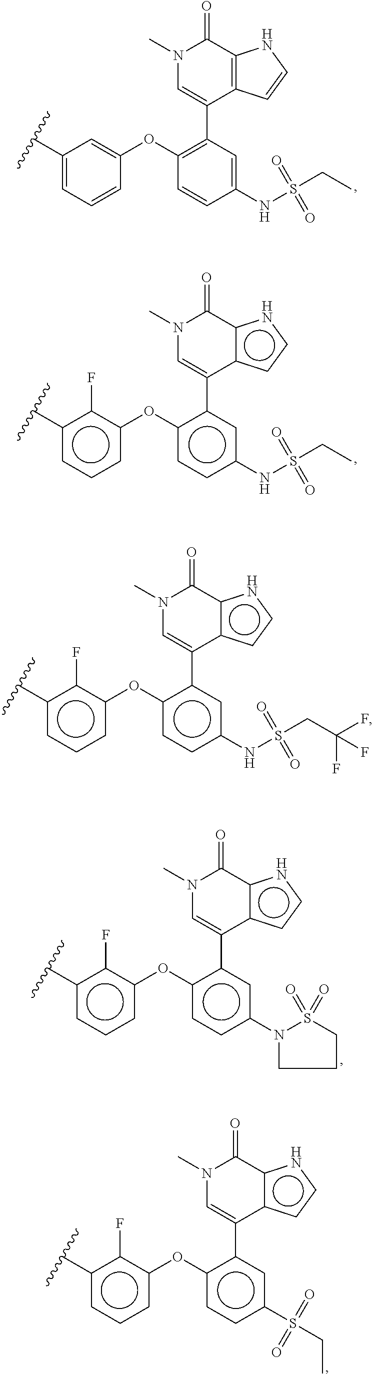

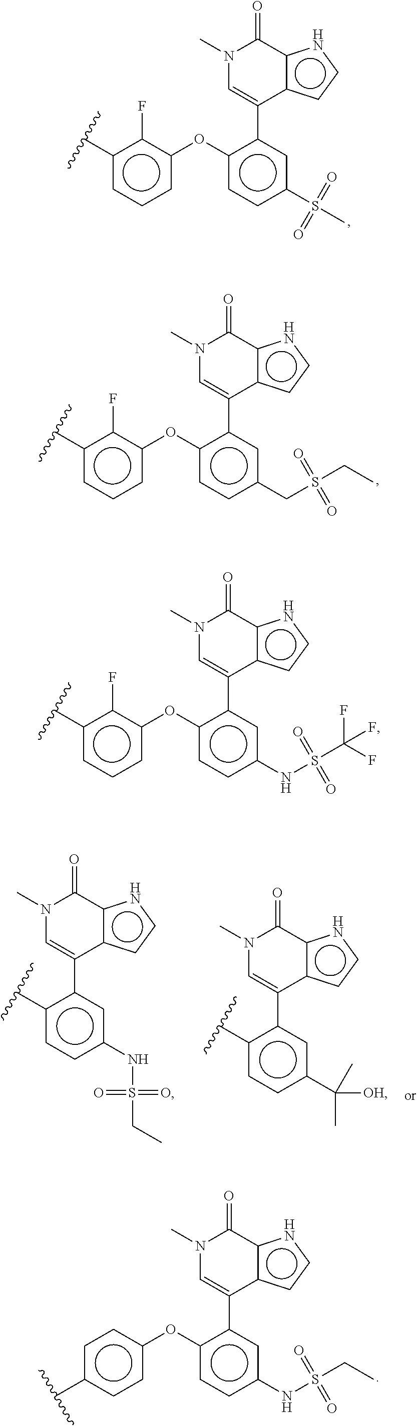

In certain embodiments, A² is

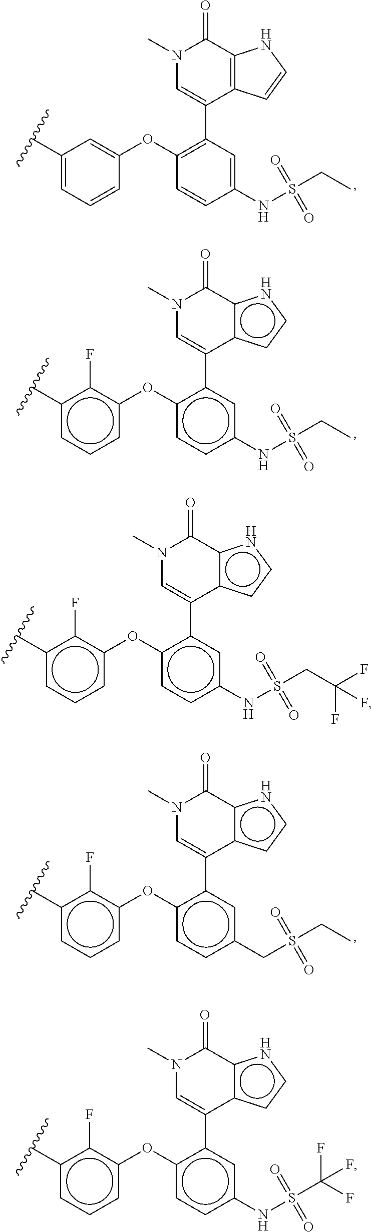

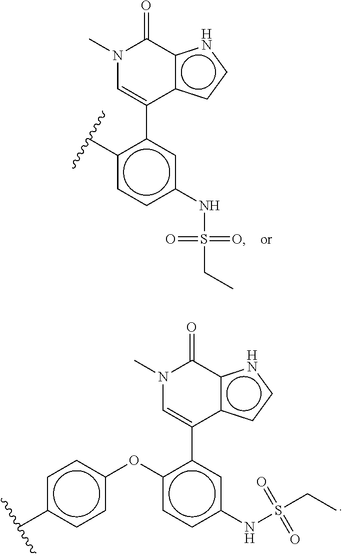

In certain embodiments A is

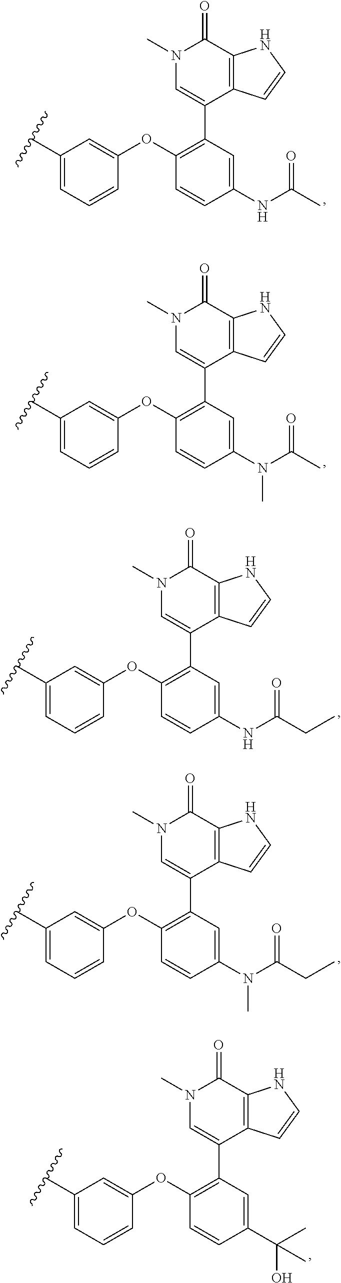

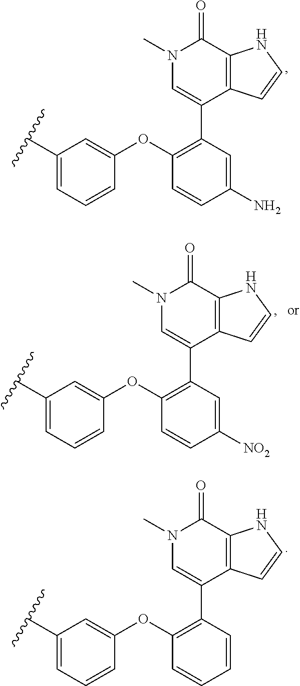

In certain embodiments, A² is selected from the groups depicted in the compounds in Table 1 below.

As defined generally above, A³ is phenylene, a 3-10 membered saturated monocyclic, bicyclic or spirocyclic carbocycylene, or C₁₋₆ alkylene. In certain embodiments, A³ is phenylene. In certain embodiments, A³ is a 3-10 membered saturated monocyclic, bicyclic or spirocyclic carbocycylene. In certain embodiments, A³ is C₁₋₆ alkylene. In certain embodiments, A³ is methylene or ethylene. In certain embodiments, A³ is a 3-6 membered saturated monocyclic carbocycylene. In certain embodiments, A³ is a 6-10 membered saturated bicyclic or spirocyclic carbocycylene. In certain embodiments, A³ is selected from the groups depicted in the compounds in Table 1 below.

As defined generally above, R^{1B} represents independently for each occurrence halo, C₁₋₄ alkyl, or C₁₋₄ haloalkyl. In certain embodiments, R^{1B} is halo. In certain embodiments, R^{1B} fluoro. In certain embodiments, R^{1B} C₁₋₄ alkyl. In certain embodiments, R^{1B} is C₁₋₄ haloalkyl. In certain embodiments, R^{1B} is selected from the groups depicted in the compounds in Table 1 below.

As defined generally above, R^{2B} is hydrogen, halo, C₁₋₄ alkyl, or C₁₋₄ haloalkyl. In certain embodiments, R^{2B} is hydrogen. In certain embodiments, R^{2B} is halo. In certain embodiments, R^{2B} is C₁₋₄ alkyl. In certain embodiments, R^{2B} is C₁₋₄ haloalkyl. In certain embodiments, R^{2B} is selected from the groups depicted in the compounds in Table 1 below.

As defined generally above, R^{3B} is —N(R^{8B})SO₂R^{9B}, —SO₂N(R^{8B})₂, —SO₂R^{9B}, —(C₁₋₆ alkylene)-SO₂R^{9B}, —(C₀₋₆ alkylene)-N(R^{8B})C(O)R^{9B}, —(C₀₋₆ alkylene)-C(O)N(R^{8B})₂, —N(R^{8B})₂, —NO₂, C₁₋₆ hydroxyalkyl, hydrogen, or a 4-7 membered saturated carbocyclic ring in which one CH₂ is replaced with SO₂. In certain embodiments, R^{3B} is —N(R^{1B})SO₂R^{9B}. In certain embodiments, R^{3B} is —SO₂R^{9B}. In certain embodiments, R^{3B} is —(C₁₋₆ alkylene)-SO₂R^{9B}. In certain embodiments, R^{3B} is —SO₂N(R^{8B})₂. In certain embodiments, R^{3B} is —(C₀₋₆ alkylene)-N(R^{8B})C(O)R^{9B}. In certain embodiments, R^{3B} is —N(R^{8B})C(O)R^{9B}. In certain embodiments, R^{3B} is —N(H)C(O)CH₃. In certain embodiments, R^{3B} is —N(CH₃)C(O)CH₃. In certain embodiments, R^{3B} is —(C₆₋₆ alkylene)-C(O)N(R^{8B})₂. In certain embodiments, R^{3B} is —C(O)N(R^{8B})₂. In certain embodiments, R^{3B} is —N(R^{8B})₂. In certain embodiments, R^{3B} is —NH₂. In certain embodiments, R^{3B} is —NO₂. In certain embodiments, R^{3B} is C₁₋₆ hydroxyalkyl. In certain embodiments, R^{3B} is —C(CH₃)₂(OH). In certain embodiments, R^{3B} is hydrogen. In certain embodiments, R^{3B} is a 4-7 membered saturated carbocyclic ring in which one CH₂ is replaced with SO₂. In certain embodiments, R^{3B} is selected from the groups depicted in the compounds in Table 1 below.

As defined generally above, R^{4B} is hydrogen, halo, or C₁₋₄ alkyl. In certain embodiments, R^{4B} is hydrogen. In certain embodiments, R^{4B} is halo. In certain embodiments, R^{4B} is C₁₋₄ alkyl. In certain embodiments, R^{4B} is selected from the groups depicted in the compounds in Table 1 below.

As defined generally above, R^{5B} is C₁₋₄ alkyl or C₃₋₄ cycloalkyl. In certain embodiments, R^{5B} is C₁₋₄ alkyl. In certain embodiments, R^{5B} is methyl. In certain embodiments, R^{5B} is C₃₋₄ cycloalkyl. In certain embodiments, R^{5B} is selected from the groups depicted in the compounds in Table 1 below.

As defined generally above, R^{6B} is hydrogen, C₁₋₄ alkyl, or C₃₋₄ cycloalkyl. In certain embodiments, R^{6B} is hydrogen. In certain embodiments, R^{6B} is C₁₋₄ alkyl. In certain embodiments, R^{6B} is C₃₋₄ cycloalkyl. In certain embodiments, R^{6B} is selected from the groups depicted in the compounds in Table 1 below.

As defined generally above, R^{7B} represents independently for each occurrence C₁₋₄ alkyl or C₃₋₄ cycloalkyl. In certain embodiments, R^{7B} is C₁₋₄ alkyl. In certain embodiments, R^{7B} is methyl. In certain embodiments, R^{7B} is C₃₋₄ cycloalkyl. In certain embodiments, R^{7B} is selected from the groups depicted in the compounds in Table 1 below.

As defined generally above, R^{8B} represents independently for each occurrence hydrogen or C₁₋₄ alkyl; or two occurrences of R^{8B} are taken together with the nitrogen atom to which they are attached to form a 3-7 membered heterocyclic ring containing 1 or 2 heteroatoms independently selected from nitrogen and oxygen; or R^{8E} and R^{9B} are taken together with their intervening atoms to a form a 5-7 membered ring containing 1, 2, or 3 heteroatoms independently selected from nitrogen, oxygen, and sulfur. In certain embodiments, R^{8B} represents independently for each occurrence hydrogen or C₁₋₄ alkyl. In certain embodiments, R^{8B} is hydrogen. In certain embodiments, R^{8B} is C₁₋₄ alkyl. In certain embodiments, two occurrences of R^{8B} are taken together with the nitrogen atom to which they are attached to form a 3-7 membered heterocyclic ring containing 1 or 2 heteroatoms independently selected from nitrogen and oxygen. In certain embodiments, R^{8B} and R^{9B} are taken together with their intervening atoms to a form a 5-7 membered ring containing 1, 2, or 3 heteroatoms independently selected from nitrogen, oxygen, and sulfur. In certain embodiments, R^{8B} is selected from the groups depicted in the compounds in Table 1 below.

As defined generally above, R^{9B} is C₁₋₆ alkyl, C₁₋₆ haloalkyl, —(C₁₋₆ alkylene)-(C₃₋₆ cycloalkyl), or C₃₋₆ cycloalkyl. In certain embodiments, R^{9B} is C₁₋₆ alkyl or C₁₋₆ haloalkyl. In certain embodiments, R^{9B} is C₁₋₆ alkyl. In certain embodiments, R^{9B} is C₁₋₆ haloalkyl. In certain embodiments, R^{9B} is —(C₁₋₆ alkylene)-(C₃₋₆ cycloalkyl). In certain embodiments, R^{9B} is C₃₋₆ cycloalkyl. In certain embodiments, R^{9B} is selected from the groups depicted in the compounds in Table 1 below.

As defined generally above, k is 1, 2, 3, or 4. In certain embodiments, k is 1. In certain embodiments, k is 2. In certain embodiments, k is 3. In certain embodiments, k is 4. In certain embodiments, k is selected from the corresponding value in the groups depicted in the compounds in Table 1 below.

As defined generally above, m, n, p and t are independently 0, 1, or 2. In certain embodiments, p is 2. In certain embodiments, p is 1. In certain embodiments, p is 0. In certain embodiments, t is 2. In certain embodiments, m is 0. In certain embodiments, m is 1. In certain embodiments, m is 2. In certain embodiments, n is 0. In certain embodiments, n is 1. In certain embodiments, n is 2. In certain embodiments, t is 0. In certain embodiments, t is 1. In certain embodiments, p is selected from the corresponding value in the groups depicted in the compounds in Table 1 below. In certain embodiments, t is selected from the corresponding value in the groups depicted in the compounds in Table 1 below. In certain embodiments, m is selected from the corresponding value in the groups depicted in the compounds in Table 1 below. In certain embodiments, n is selected from the corresponding value in the groups depicted in the compounds in Table 1 below.

In certain embodiments, the compound of Formula I is further defined by Formula Ia or a pharmaceutically acceptable salt thereof:

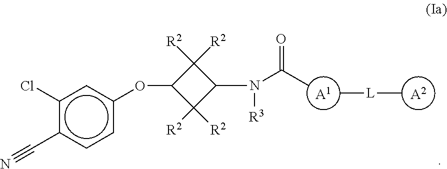

In certain embodiments, the definition of variables R², R³, A¹, and A² is one of the embodiments described above in connection with Formula I.

In certain embodiments, the compound of Formula I is further defined by Formula Ib or a pharmaceutically acceptable salt thereof:

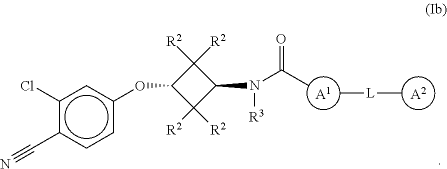

In certain embodiments, the definition of variables R², R², A¹, and A² is one of the embodiments described above in connection with Formula I.

In certain embodiments, the compound of Formula I is further defined by Formula Ic or a pharmaceutically acceptable salt thereof:

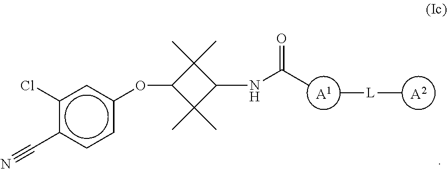

In certain embodiments, the definition of variables A¹ and A² is one of the embodiments described above in connection with Formula I.

In certain embodiments, the compound of Formula I is further defined by Formula Id or a pharmaceutically acceptable salt thereof:

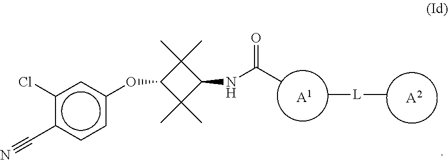

In certain embodiments, the definition of variables A¹ and A² is one of the embodiments described above in connection with Formula I.

In certain embodiments, the compound of Formula I is further defined by Formula Ie:

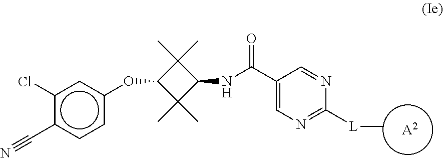

or a pharmaceutically acceptable salt thereof, wherein

### A² is

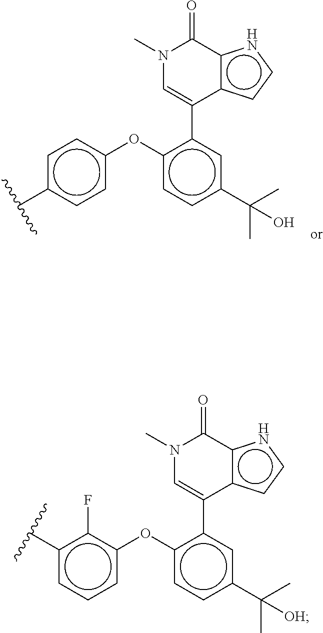

and
L is a linker.

The compounds may be further characterized according to, for example, the identity of L. Exemplary further embodiments for L are provided in Part C below.

In certain embodiments, the compound of Formula I is further defined by Formula If:

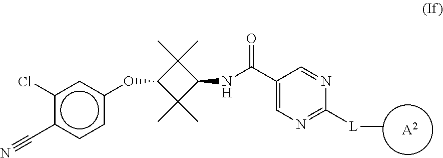

or a pharmaceutically acceptable salt thereof, wherein

### A² is

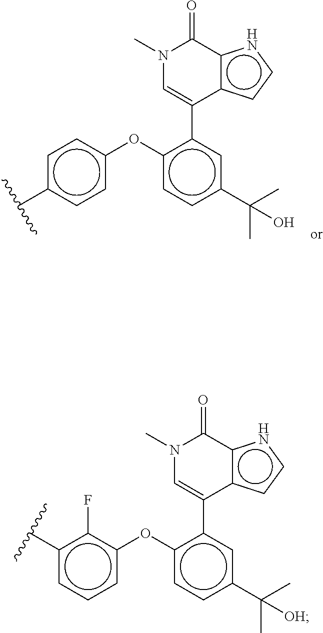

and
L is one of the following:
(3-7 membered, monocyclic, saturated heterocyclic ring containing 1 or 2 heteroatoms selected from nitrogen)-C₁₋₁₀ alkylene, wherein *** is the point of attachment to A²;
(piperidinylene)-X¹—***, wherein *** is the point of attachment to A², and X¹ is (i) C₁₋₅ alkylene where 1 or 2 methylene groups are optionally replaced by —O—, —N(H)—, or —N(C₁₋₄ alkyl)-;
7-11 membered spirocyclic or fused bicyclic saturated heterocyclic ring containing 1, 2, or 3 heteroatoms selected from nitrogen and oxygen; or
(3-7 membered monocyclic saturated heterocyclic ring containing 1 or 2 heteroatoms selected from nitrogen)-X³-(3-7 membered monocyclic saturated heterocyclic ring containing 1 or 2 heteroatoms selected from nitrogen)-, wherein X³ is C₁₋₁₀ alkylene, —O—, —N(H)—, —N(C₁₋₄ alkyl)-, or a bond.

In certain embodiments, A² is

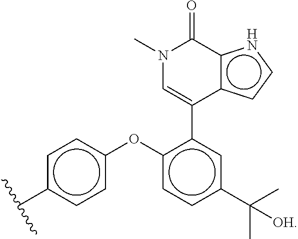

In certain embodiments, A² is

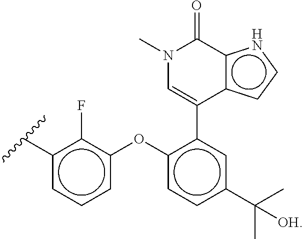

In certain embodiments, L is -(3-7 membered, monocyclic, saturated heterocyclic ring containing 1 or 2 heteroatoms selected from nitrogen)-C₁₋₁₀ alkylene, wherein *** is the point of attachment to A². In certain embodiments, L is -(piperidinylene)-X¹—***, wherein *** is the point of attachment to A², and X¹ is (i) C₁₋₅ alkylene where 1 or 2 methylene groups are optionally replaced by —O—, —N(H)—, or —N(C₁₋₄ alkyl)-. In certain embodiments, L is 7-11 membered spirocyclic or fused bicyclic saturated heterocyclic ring containing 1, 2, or 3 heteroatoms selected from nitrogen and oxygen. In certain embodiments, L is -(3-7 membered monocyclic saturated heterocyclic ring containing 1 or 2 heteroatoms selected from nitrogen)-X³-(3-7 membered monocyclic saturated heterocyclic ring containing 1 or 2 heteroatoms selected from nitrogen)-, wherein X³ is C₁₋₁₀ alkylene, —O—, —N(H)—, —N(C₁₋₄ alkyl)-, or a bond.

In certain embodiments, in connection with Formula If, L is -piperidinylene-(C₁₋₅ alkylene)-***, -(piperidinylene)-X³-(azetidinylene)-, or -(azetidinylene)-X³-(azetidinylene)-, wherein X³ is C₁₋₃ alkylene, and *** is the point of attachment to A².

In certain embodiments, the compound of Formula I is further defined by Formula Ig:

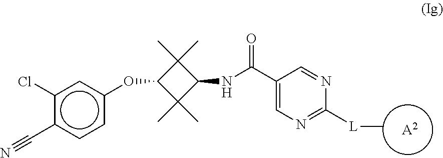

or a pharmaceutically acceptable salt thereof; wherein
A² is

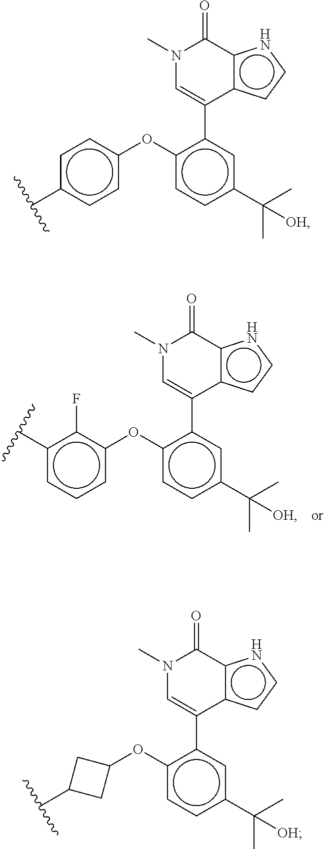

and
L is one of the following:
   -(3-7 membered, monocyclic, saturated heterocyclic ring containing 1 or 2 heteroatoms selected from nitrogen)-C₁₋₁₀ alkylene, wherein *** is the point of attachment to A²;
   -(piperidinylene)-X¹—***, wherein *** is the point of attachment to A², and X¹ is (i) C₁₋₅ alkylene where 1 or 2 methylene groups are optionally replaced by —O—, —N(H)—, or —N(C₁₋₄ alkyl)-;
   -(7-11 membered spirocyclic or fused bicyclic saturated heterocyclic ring containing 1, 2, or 3 heteroatoms selected from nitrogen and oxygen)-(C₀₋₆ alkylene)-***, wherein *** is the point of attachment to A²;
   -(3-7 membered monocyclic saturated heterocyclic ring containing 1 or 2 heteroatoms selected from nitrogen)-X³-(3-7 membered monocyclic saturated heterocyclic ring containing 1 or 2 heteroatoms selected from nitrogen)-, wherein X³ is C₁₋₁₀ alkylene, —O—, —N(H)—, —N(C₁₋₄ alkyl)-, or a bond; or
   -(3-7 membered monocyclic saturated heterocyclic ring containing 1 or 2 heteroatoms selected from nitrogen)-X³—(C₃₋₆ cycloalkylene)-***, wherein *** is the point of attachment to A², and wherein X³ is C₁₋₁₀ alkylene, —O—, —N(H)—, —N(C₁₋₄ alkyl)-, or a bond.

In certain embodiments, L is A² is

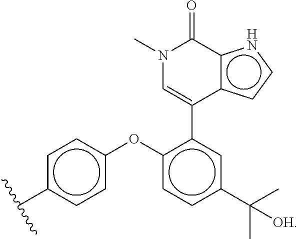

In certain embodiments, L is A² is

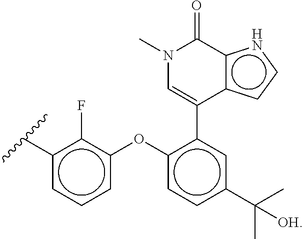

In certain embodiments, L is A² is

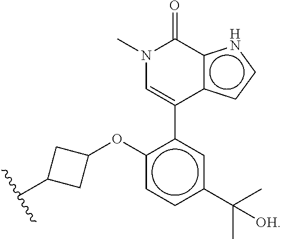

In certain embodiments, L is -(3-7 membered, monocyclic, saturated heterocyclic ring containing 1 or 2 heteroatoms selected from nitrogen)-C₁₋₁₀ alkylene, wherein *** is the point of attachment to A². In certain embodiments, L is -(piperidinylene)-X¹—***, wherein *** is the point of attachment to A², and X¹ is (i) C₁₋₅ alkylene where 1 or 2 methylene groups are optionally replaced by —O—, —N(H)—, or —N(C₁₋₄ alkyl)-. In certain embodiments, L is -(7-11 membered spirocyclic or fused bicyclic saturated heterocyclic ring containing 1, 2, or 3 heteroatoms selected from nitrogen and oxygen)-(C₀₋₆ alkylene)-***, wherein *** is the point of attachment to A². In certain embodiments, L is -(3-7 membered monocyclic saturated heterocyclic ring containing 1 or 2 heteroatoms selected from nitrogen)-X³-(3-7 membered monocyclic saturated heterocyclic ring containing 1 or 2 heteroatoms selected from nitrogen)-, wherein X³ is C₁₋₁₀ alkylene, —O—, —N(H)—, —N(C₁₋₄ alkyl)-, or a bond. In certain embodiments, L is -(3-7 membered monocyclic saturated heterocyclic ring containing 1 or 2 heteroatoms selected from nitrogen)-X³—(C₃₋₆ cycloalkylene)-***, wherein *** is the point of attachment to A², and wherein X³ is C₁₋₁₀ alkylene, —O—, —N(H)—, —N(C₁₋₄ alkyl)-, or a bond.

In certain embodiments, in connection with Formula Ig, L is -piperidinylene-(C₁₋₅ alkylene)-***, -(piperidinylene)-X³-(azetidinylene)-, or -(azetidinylene)-X³-(azetidinylene)-, wherein X³ is C₁₋₃ alkylene, and *** is the point of attachment to A².

### Part B: Compound of Formula I-1

One aspect of the invention provides a compound represented by Formula I-1:

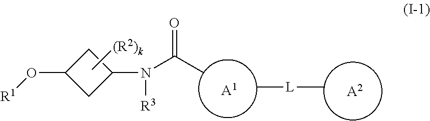

or a pharmaceutically acceptable salt thereof, wherein:
R¹ is phenyl substituted by cyano, halogen, and m occurrences of R⁴;
R² represents independently for each occurrence C₁₋₄ alkyl;
R³ is hydrogen or C₁₋₄ alkyl;
R⁴ is C₁₋₄ alkyl;
R⁵ represents independently for each occurrence C₁₋₄ alkyl or halogen, or one occurrence of R⁵ is taken together with R³ to form a C₁₋₃ alkylene;
A¹ is a pyridazinylene, pyrimidinylene, pyrazinylene, pyridinylene, phenylene, or C₅0.6 cycloalkylene, each of which is substituted with n occurrences of R⁵;
A² is one of the following:

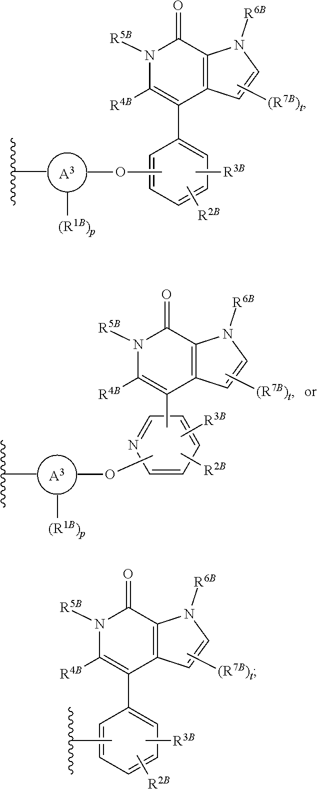

A³ is phenylene, pyridinylene, pyrazinylene, a 3-10 membered saturated monocyclic, bicyclic or spirocyclic carbocycylene, or C₁₋₆ alkylene;
R^{1B} represents independently for each occurrence halo, C₁₋₄ alkyl, or C₁₋₄ haloalkyl;
R^{2B} is hydrogen, halo, C₁₋₄ alkyl, or C₁₋₄ haloalkyl;
R^{3B} is —N(R^{8B})SO₂R^{9B}, —SO₂N(R^{8B})₂, —SO₂R^{9B}, —(C₁₋₆ alkylene)-SO₂R^{9B}, —(C₀₋₆ alkylene)-N(R^{8B})C(O)R^{9B}, —(C₀₋₆ alkylene)-C(O)N(R^{8B})₂, —N(R^{8B})₂, —NO₂, C₁₋₆ hydroxyalkyl, hydrogen, or a 4-7 membered saturated carbocyclic ring in which one CH₂ is replaced with SO₂;
R^{4B} is hydrogen, halo, or C₁₋₄ alkyl;
R^{5B} is C₁₋₄ alkyl or C₃₋₄ cycloalkyl;
R^{6B} is hydrogen, C₁₋₄ alkyl, or C₃₋₄ cycloalkyl;
R^{7B} represents independently for each occurrence C₁₋₄ alkyl or C₃₋₄ cycloalkyl;
R^{8B} represents independently for each occurrence hydrogen or C₁₋₄ alkyl; or two occurrences of R^{8B} are taken together with the nitrogen atom to which they are attached to form a 3-7 membered heterocyclic ring containing 1 or 2 heteroatoms independently selected from nitrogen and oxygen; or R^{8B} and R^{9B} are taken together with their intervening atoms to a form a 5-7 membered ring containing 1, 2, or 3 heteroatoms independently selected from nitrogen, oxygen, and sulfur;
R^{9B} is C₁₋₆ alkyl, C₁₋₆ haloalkyl, —(C₁₋₆ alkylene)-(C₃₋₆ cycloalkyl), or C₃₋₆ cycloalkyl; and
L is a linker;
k is 1, 2, 3, or 4; and
m, n, p and t are independently 0, 1, or 2.

The definitions of variables in Formula I-1 above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I-1.

As define generally above, R¹ is phenyl substituted by cyano, halogen, and m occurrences of R⁴. In certain embodiments, R¹ is phenyl substituted by cyano, halogen, and 0 occurrences of R⁴. In certain embodiments, R¹ is phenyl substituted by cyano, halogen, and 1 occurrence of R⁴. In certain embodiments, R¹ is phenyl substituted by cyano, halogen, and 2 occurrences of R⁴. In certain embodiments, R¹ is phenyl substituted by cyano, Cl, and 0 occurrences of R⁴. In certain embodiments, R¹ is

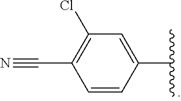 [SMILES: *c1ccc(C#N)c(Cl)c1]

In certain embodiments, R¹ is selected from the groups depicted in the compounds in Table 1 below.

As defined generally above, R² represents independently for each occurrence C₁₋₄ alkyl. In certain embodiments, R² is C₁₋₂ alkyl. In certain embodiments, R² is methyl. In certain embodiments, R² is selected from the groups depicted in the compounds in Table 1 below.

As defined generally above, R³ is hydrogen or C₁₋₄ alkyl. In certain embodiments, R³ is hydrogen. In certain embodiments, R³ is C₁₋₄ alkyl. In certain embodiments, R³ is selected from the groups depicted in the compounds in Table 1 below.

As defined generally above, R⁴ is C₁₋₄ alkyl. In certain embodiments, R⁴ is methyl. In certain embodiments, R⁴ is selected from the groups depicted in the compounds in Table 1 below.

As defined generally above, R⁵ represents independently for each occurrence C₁₋₄ alkyl or halogen, or one occurrence of R⁵ is taken together with R³ to form a C₁₋₃ alkylene. In certain embodiments, R⁵ represents independently for each occurrence C₁₋₄ alkyl. In certain embodiments, R⁵ represents independently for each occurrence C₁₋₄ alkyl or halogen. In certain embodiments, R⁵ represents independently for each occurrence methyl. In certain embodiments, R⁵ represents independently for each occurrence halogen. In certain embodiments, R⁵ represents independently for each occurrence Cl, Br, or F.

In certain embodiments, R⁵ is C₁₋₄ alkyl. In certain embodiments, R⁵ is methyl. In certain embodiments, R⁵ is halogen. In certain embodiments, R⁵ is Cl, Br, or F. In certain embodiments, one occurrence of R⁵ is taken together with R³ to form a C₁₋₃ alkylene. In certain embodiments, R⁵ is selected from the groups depicted in the compounds in Table 1 below.

As defined generally above, A¹ is a pyridazinylene, pyrimidinylene, pyrazinylene, pyridinylene, phenylene, or C₅₋₆ alkylene, each of which is substituted with n occurrences of R⁵. In certain embodiments, A¹ is a pyridazinylene, pyrimidinylene, pyrazinylene, pyridinylene, or phenylene, each of which is substituted with n occurrences of R⁵. In certain embodiments, A¹ is pyridazinylene substituted with n occurrences of R⁵. In certain embodiments, A¹ is

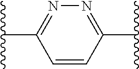 [SMILES: *c1ccc(*)nn1]

In certain embodiments, A¹ is pyrimidinylene substituted with n occurrences of R⁵. In certain embodiments, A¹ is

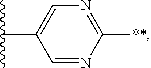

where ** is the point of attachment to L. In certain embodiments, A¹ is

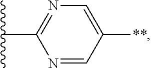

where ** is the point of attachment to L. In certain embodiments, A is pyrazinylene substituted with n occurrences of R⁵. In certain embodiments, A¹ is

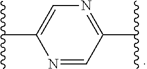 [SMILES: *c1cnc(*)cn1]

In certain embodiments, A¹ is pyridinylene substituted with n occurrences of R⁵. In certain embodiments, A¹ is

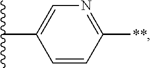

where ** is the point of attachment to L. In certain embodiments, A¹ is phenylene substituted with n occurrences of R⁵. In certain embodiments, A¹ is

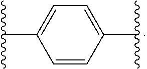 [SMILES: *c1ccc(*)cc1]

In certain embodiments, A¹ is C₅₋₆ cycloalkylene substituted with n occurrences of R⁵. In certain embodiments, A¹ is C₅₋₆ cycloalkylene substituted with n occurrences of R⁵. In certain embodiments, A¹ is a pyridazinylene substituted with 0 occurrences of R⁵. In certain embodiments, A¹ is a pyrimidinylene substituted with 0 occurrences of R⁵. In certain embodiments, A¹ is a pyrazinylene substituted with 0 occurrences of R⁵. In certain embodiments, A¹ is a pyridinylene substituted with 0 occurrences of R⁵. In certain embodiments, A¹ is a phenylene substituted with 0 occurrences of R⁵. In certain embodiments, A¹ is C₅₋₆ cycloalkylene substituted with 0 occurrences of R⁵. In certain embodiments, A¹ is C₆ cycloalkylene substituted with 0 occurrences of R⁵. In certain embodiments, A¹ is selected from the groups depicted in the compounds in Table 1 below.

As defined generally above, A^{2 1} is

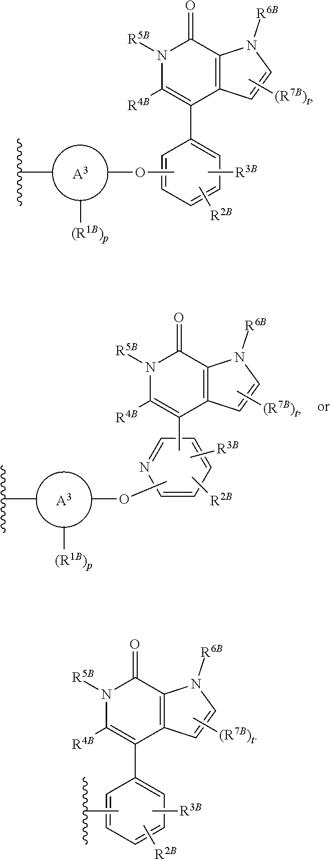

In certain embodiments, A² is

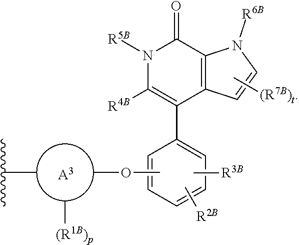

In certain embodiments, A² is

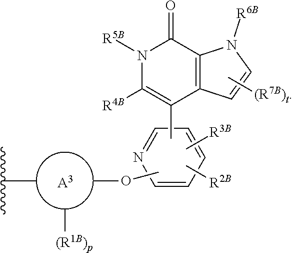

In certain embodiments, A² is

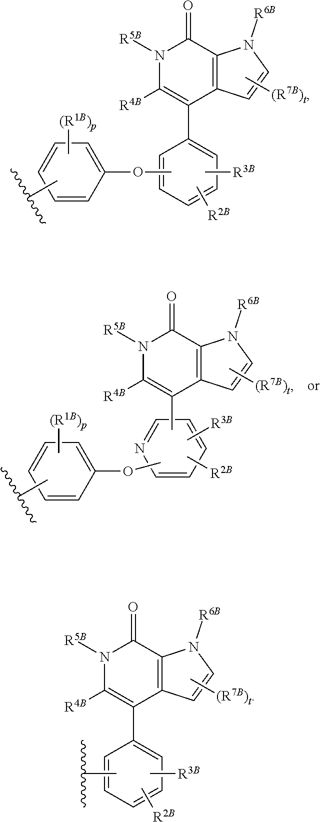

In certain embodiments, A² is

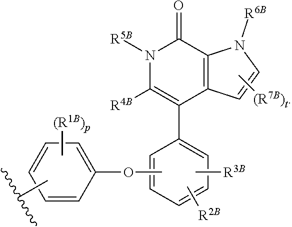

In certain embodiments, A² is

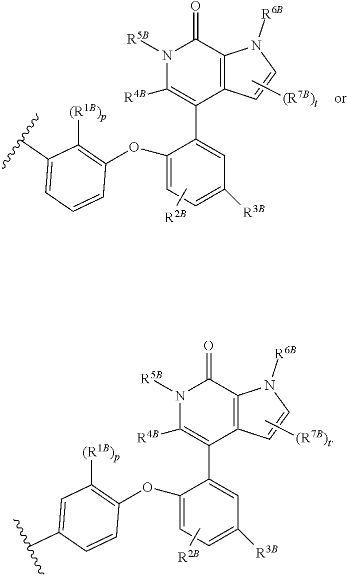

In certain embodiments, A² is

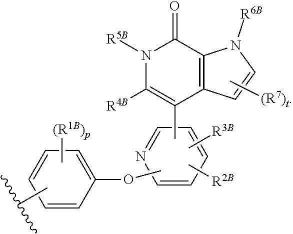

In certain embodiments, A² is

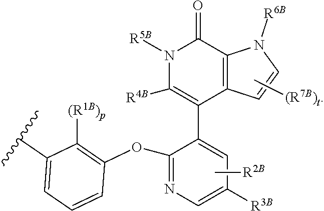

In certain embodiments, A² is

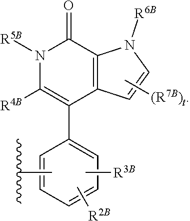

In certain embodiments, A² is

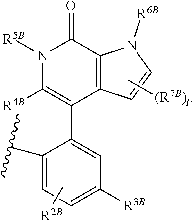

In certain embodiments, A² is

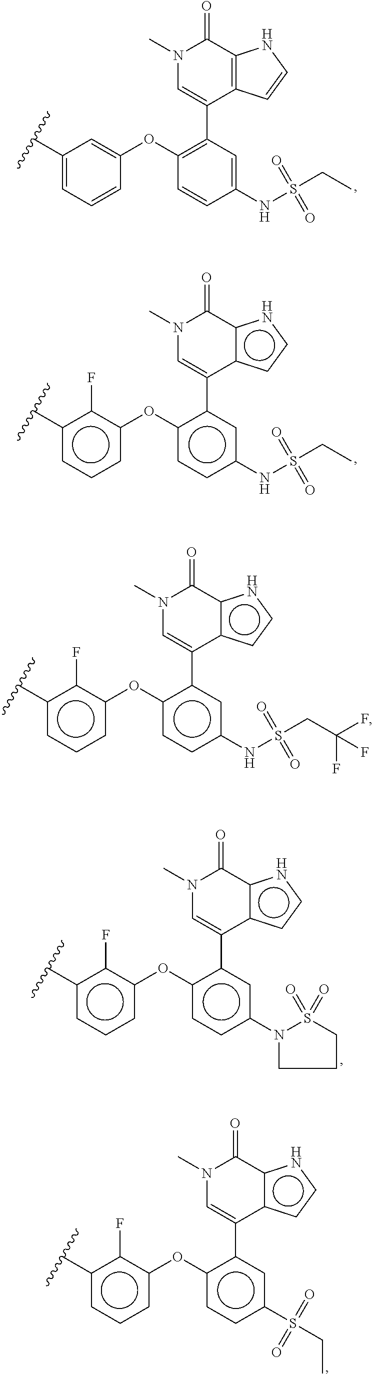

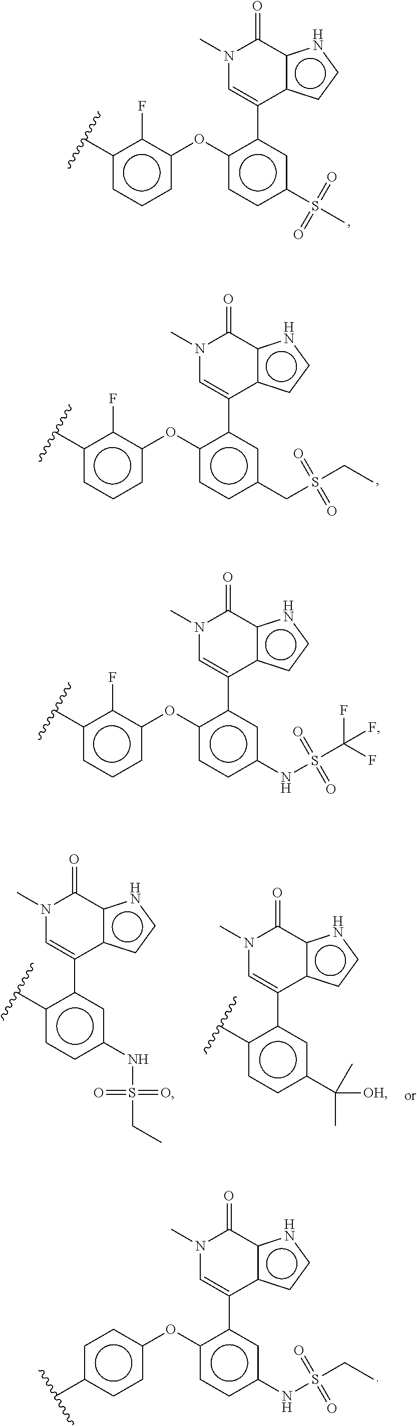

In certain embodiments, A² is

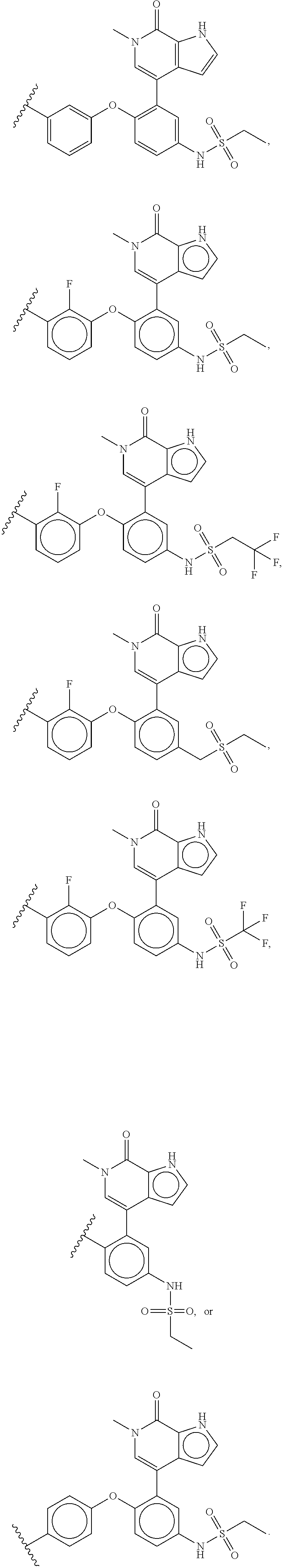

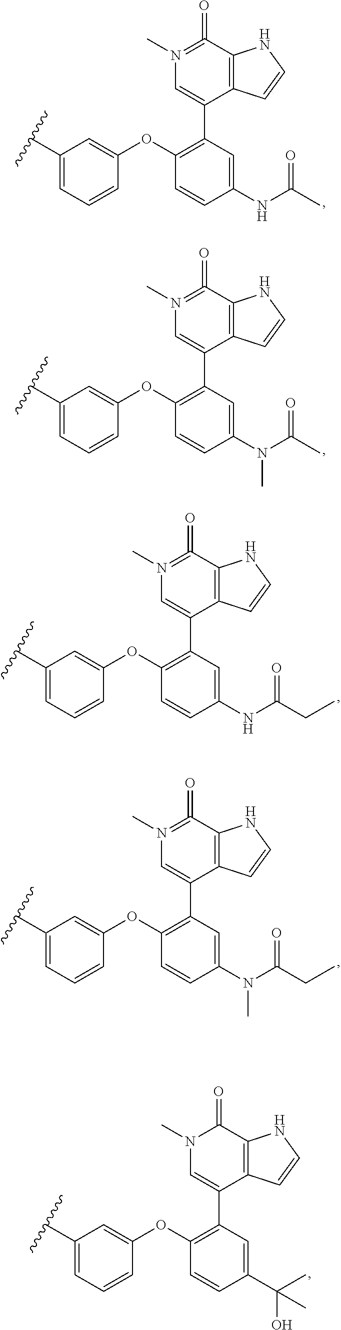

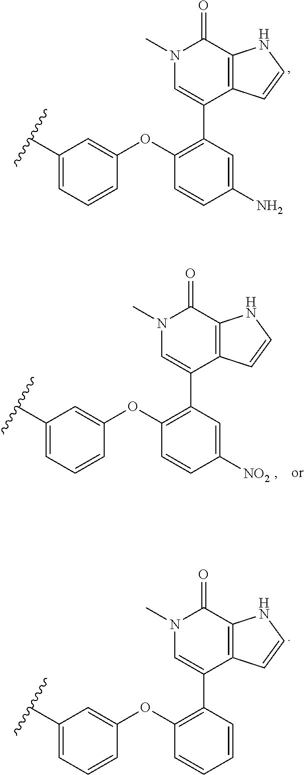 [SMILES: *c1cccc(Oc2ccc(N)cc2-c2cn(C)c(=O)c3[nH]ccc23)c1 ; *c1cccc(Oc2ccc([N+](=O)[O-])cc2-c2cn(C)c(=O)c3[nH]ccc23)c1 ; *c1cccc(Oc2ccccc2-c2cn(C)c(=O)c3[nH]ccc23)c1]

In certain embodiments, A² is

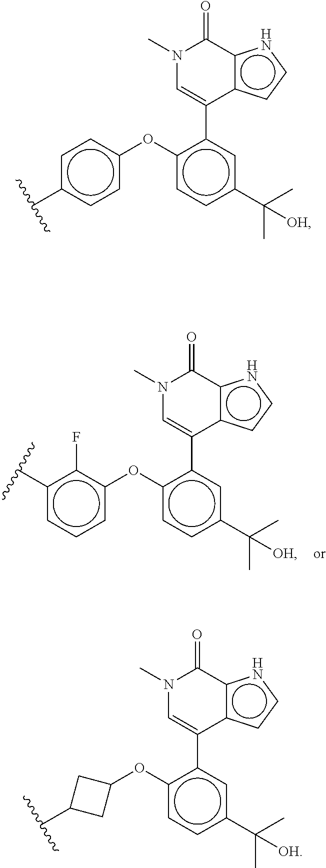

In certain embodiments, A² is selected from the groups depicted in the compounds in Table 1 below.

As defined generally above, A³ is phenylene, pyridinylene, pyrazinylene, a 3-10 membered saturated monocyclic, bicyclic or spirocyclic carbocycylene, or C₁₋₆ alkylene. In certain embodiments, A³ is phenylene, a 3-10 membered saturated monocyclic, bicyclic or spirocyclic carbocycylene, or C₁₋₆ alkylene. In certain embodiments, A³ is phenylene. In certain embodiments, A³ is pyridinylene. In certain embodiments, A³ is pyrazinylene. In certain embodiments, A³ is a 3-10 membered saturated monocyclic, bicyclic or spirocyclic carbocycylene. In certain embodiments, A³ is C₁₋₆ alkylene. In certain embodiments, A³ is methylene or ethylene. In certain embodiments, A³ is a 3-6 membered saturated monocyclic carbocycylene. In certain embodiments, A³ is a 6-10 membered saturated bicyclic or spirocyclic carbocycylene. In certain embodiments, A³ is selected from the groups depicted in the compounds in Table 1 below.

As defined generally above, R^{1B} represents independently for each occurrence halo, C₁₋₄ alkyl, or C₁₋₄ haloalkyl. In certain embodiments, R^{1B} is halo. In certain embodiments, R^{1B} is fluoro. In certain embodiments, R^{1B} is C₁₋₄ alkyl. In certain embodiments, R^{1B} is C₁₋₄ haloalkyl. In certain embodiments, R^{1B} is selected from the groups depicted in the compounds in Table 1 below.

As defined generally above, R^{2B} is hydrogen, halo, C₁₋₄ alkyl, or C₁₋₄ haloalkyl. In certain embodiments, R^{2B} is hydrogen. In certain embodiments, R^{2B} is halo. In certain embodiments, R^{2B} is C₁₋₄ alkyl. In certain embodiments, R^{2B} is C₁₋₄ haloalkyl. In certain embodiments, R^{2B} is selected from the groups depicted in the compounds in Table 1 below.

As defined generally above, R^{3B} is —N(R^{8B})SO₂R^{9B}, —SO₂N(R^{8B})₂, —SO₂R^{9B}, —(C₁₋₆ alkylene)-SO₂R^{9B}, —(C₀₋₆ alkylene)-N(R^{8B})C(O)R^{9B}, —(C_{0.6} alkylene)-C(O)N(R^{RB})₂, —N(R^{8B})₂, —NO₂, C₁₋₆ hydroxyalkyl, hydrogen, or a 4-7 membered saturated carbocyclic ring in which one CH₂ is replaced with SO₂. In certain embodiments, R^{3B} is —N(R^{8B})SO₂R^{9B}. In certain embodiments, R^{3B} is —SO₂R^{9B}. In certain embodiments, R^{3B} is —(C₁₋₆ alkylene)-SO₂R^{9B}. In certain embodiments, R^{3B} is —SO₂N(R^{3B})₂. In certain embodiments, R^{3B} is —(C₀₋₆ alkylene)-N(R^{8B})C(O)R^{9B}. In certain embodiments, R^{3B} is —N(R^{8B})C(O)R^{9B}. In certain embodiments, R^{3B} is —N(H)C(O)CH₃. In certain embodiments, R^{3B} is —N(CH₃)C(O)CH₃. In certain embodiments, R^{3B} is —(C_{0.6} alkylene)-C(O)N(R^{3B})₂. In certain embodiments, R^{3B} is —C(O)N(R^{3B})₂. In certain embodiments, R^{3B} is —N(R^{8B})₂. In certain embodiments, R^{3B} is —NH₂. In certain embodiments, R^{3B} is —NO₂. In certain embodiments, R^{3B} is C₁₋₆ hydroxyalkyl. In certain embodiments, R^{3B} is —C(CH₃)₂(OH). In certain embodiments, R^{3B} is hydrogen. In certain embodiments, R^{3B} is a 4-7 membered saturated carbocyclic ring in which one CH₂ is replaced with SO₂. In certain embodiments, R^{3B} is selected from the groups depicted in the compounds in Table 1 below.

As defined generally above, R^{4B} is hydrogen, halo, or C₁₋₄ alkyl. In certain embodiments, R^{4B} is hydrogen. In certain embodiments, R^{4B} is halo. In certain embodiments, R^{4B} is C₁₋₄ alkyl. In certain embodiments, R^{4B} is selected from the groups depicted in the compounds in Table 1 below.

As defined generally above, R^{5B} is C₁₋₄ alkyl or C₃₋₄ cycloalkyl. In certain embodiments, R^{5B} is C₁₋₄ alkyl. In certain embodiments, R^{5B} is methyl. In certain embodiments, R^{5B} is C₃₋₄ cycloalkyl. In certain embodiments, R^{5B} is selected from the groups depicted in the compounds in Table 1 below.

As defined generally above, R^{6B} is hydrogen, C₁₋₄ alkyl, or C₃₋₄ cycloalkyl. In certain embodiments, R^{6B} is hydrogen. In certain embodiments, R^{6B} is C₁₋₄ alkyl. In certain embodiments, R^{6B} is C₃₋₄ cycloalkyl. In certain embodiments, R^{6B} is selected from the groups depicted in the compounds in Table 1 below.

As defined generally above, R^{7B} represents independently for each occurrence C₁₋₄ alkyl or C₃₋₄ cycloalkyl. In certain embodiments, R^{7B} is C₁₋₄ alkyl. In certain embodiments, R^{7B} is methyl. In certain embodiments, R^{7B} is C₃₋₄ cycloalkyl. In certain embodiments, R^{7B} is selected from the groups depicted in the compounds in Table 1 below.

As defined generally above, R^{8B} represents independently for each occurrence hydrogen or C₁₋₄ alkyl; or two occurrences of R^{8B} are taken together with the nitrogen atom to which they are attached to form a 3-7 membered heterocyclic ring containing 1 or 2 heteroatoms independently selected from nitrogen and oxygen; or R^{8B} and R^{9B} are taken together with their intervening atoms to a form a 5-7 membered ring containing 1, 2, or 3 heteroatoms independently selected from nitrogen, oxygen, and sulfur. In certain embodiments, R^{8B} represents independently for each occurrence hydrogen or C₁₋₄ alkyl. In certain embodiments, R^{8B} is hydrogen. In certain embodiments, R^{8B} is C₁-alkyl. In certain embodiments, two occurrences of R^{8B} are taken together with the nitrogen atom to which they are attached to form a 3-7 membered heterocyclic ring containing 1 or 2 heteroatoms independently selected from nitrogen and oxygen. In certain embodiments, R^{8B} and R^{9B} are taken together with their intervening atoms to a form a 5-7 membered ring containing 1, 2, or 3 heteroatoms independently selected from nitrogen, oxygen, and sulfur. In certain embodiments, R^{8B} is selected from the groups depicted in the compounds in Table 1 below.

As defined generally above, R^{9B} is C₁₋₆ alkyl, C₁₋₆ haloalkyl, —(C₁₋₆ alkylene)-(C₃₋₆ cycloalkyl), or C₃₋₆ cycloalkyl. In certain embodiments, R^{9B} is C₁₋₆ alkyl or C₁₋₆ haloalkyl. In certain embodiments, R^{9B} is C₁₋₆ alkyl. In certain embodiments, R^{9B} is C₁₋₆ haloalkyl. In certain embodiments, R^{9B} is —(C₁₋₆ alkylene)-(C₃₋₆ cycloalkyl). In certain embodiments, R^{9E} is C₃₋₆ cycloalkyl. In certain embodiments, R^{9B} is selected from the groups depicted in the compounds in Table 1 below.

As defined generally above, k is 1, 2, 3, or 4. In certain embodiments, k is 1. In certain embodiments, k is 2. In certain embodiments, k is 3. In certain embodiments, k is 4. In certain embodiments, k is selected from the corresponding value in the groups depicted in the compounds in Table 1 below.

As defined generally above, m, n, p and t are independently 0, 1, or 2. In certain embodiments, p is 2. In certain embodiments, p is 1. In certain embodiments, p is 0. In certain embodiments, t is 2. In certain embodiments, m is 0. In certain embodiments, m is 1. In certain embodiments, m is 2. In certain embodiments, n is 0. In certain embodiments, n is 1. In certain embodiments, n is 2. In certain embodiments, t is 0. In certain embodiments, t is 1. In certain embodiments, p is selected from the corresponding value in the groups depicted in the compounds in Table 1 below. In certain embodiments, t is selected from the corresponding value in the groups depicted in the compounds in Table 1 below. In certain embodiments, m is selected from the corresponding value in the groups depicted in the compounds in Table 1 below. In certain embodiments, n is selected from the corresponding value in the groups depicted in the compounds in Table 1 below.

In certain embodiments, the compound of Formula 1-1 is further defined by Formula Ia or a pharmaceutically acceptable salt thereof:

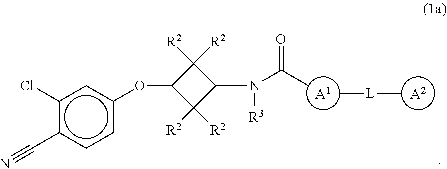

In certain embodiments, the definition of variables R², R³, A¹, and A² is one of the embodiments described above in connection with Formula I-1.

In certain embodiments, the compound of Formula I-1 is further defined by Formula Ib or a pharmaceutically acceptable salt thereof:

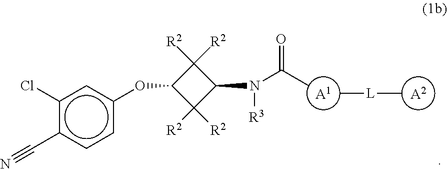

In certain embodiments, the definition of variables R², R³, A¹, and A² is one of the embodiments described above in connection with Formula I-1.

In certain embodiments, the compound of Formula I-1 is further defined by Formula Ic or a pharmaceutically acceptable salt thereof:

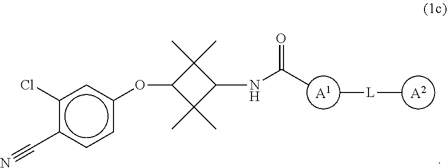

In certain embodiments, the definition of variables A¹ and A² is one of the embodiments described above in connection with Formula I-1.

In certain embodiments, the compound of Formula I-1 is further defined by Formula Id or a pharmaceutically acceptable salt thereof:

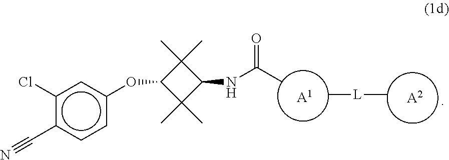

In certain embodiments, the definition of variables A¹ and A² is one of the embodiments described above in connection with Formula I-1.

The compounds may be further characterized according to, for example, the identity of L. Exemplary further embodiments for L are provided in Part F below.

Another aspect of the invention provides a compound represented by Formula I-la:

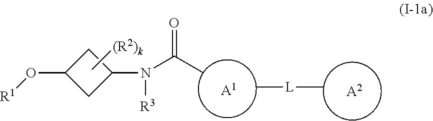

or a pharmaceutically acceptable salt thereof, wherein:
R¹ is phenyl substituted by cyano, halogen, and m occurrences of R⁴;
R² represents independently for each occurrence C₁₋₄ alkyl;
R³ is hydrogen or C₁₋₄ alkyl;
R⁴ is C₁₋₄ alkyl;
R⁵ represents independently for each occurrence C₁₋₄ alkyl or halogen, or one occurrence of R⁵ is taken together with R³ to form a C₁₋₃ alkylene;
A¹ is a pyridazinylene, pyrimidinylene, pyrazinylene, pyridinylene, phenylene, or C₅0.6 cycloalkylene, each of which is substituted with n occurrences of R⁵;
A² is one of the following:

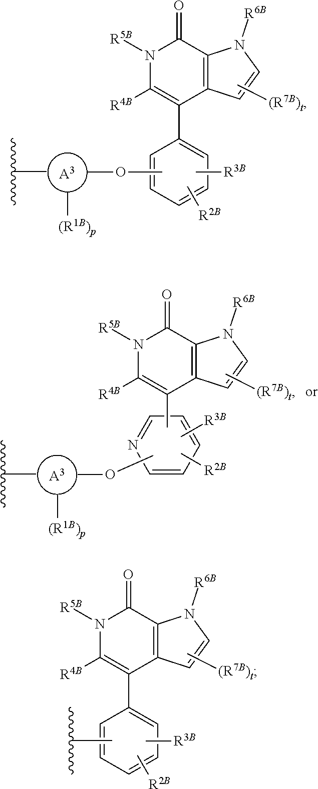

A³ is phenylene, pyridinylene, pyrazinylene, a 3-10 membered saturated monocyclic, bicyclic or spirocyclic carbocycylene, or C₁₋₆ alkylene;
R^{1B} represents independently for each occurrence halo, C₁₋₄ alkyl, or C₁₋₄ haloalkyl;
R^{2B} is hydrogen, halo, C₁₋₄ alkyl, or C₁₋₄ haloalkyl;
R^{1B} is —N(R^{8B})SO₂R^{9B}, —SO₂N(R^{8B})₂, —SO₂R^{9B}, —(C₁₋₆ alkylene)-SO₂R^{9B}, —(C₀₋₆ alkylene)-N(R^{8B})C(O)R^{9B}, —(C₀₋₆ alkylene)-C(O)N(R^{8B})₂, —N(R^{8B})₂, —NO₂, C₁₋₆ hydroxyalkyl, hydrogen, or a 4-7 membered saturated carbocyclic ring in which one CH₂ is replaced with SO₂;
R^{4B} is hydrogen, halo, or C₁₋₄ alkyl;
R^{5B} is C₁₋₄ alkyl or C₃₋₄ cycloalkyl;
R^{6B} is hydrogen, C₁₋₄ alkyl, or C₃₋₄ cycloalkyl;
R^{7B} represents independently for each occurrence C₁₋₄ alkyl or C₃₋₄ cycloalkyl;
R^{8B} represents independently for each occurrence hydrogen or C₁₋₄ alkyl; or two occurrences of R^{8B} are taken together with the nitrogen atom to which they are attached to form a 3-7 membered heterocyclic ring containing 1 or 2 heteroatoms independently selected from nitrogen and oxygen; or R^{8B} and R^{9B} are taken together with their intervening atoms to a form a 5-7 membered ring containing 1, 2, or 3 heteroatoms independently selected from nitrogen, oxygen, and sulfur;
R^{9B} is C₁₋₆ alkyl, C₁₋₆ haloalkyl, —(C₁₋₆ alkylene)-(C₃₋₆ cycloalkyl), or C₃₋₆ cycloalkyl; and
L is one of the following:
a 7-11 membered spirocyclic or fused bicyclic saturated heterocyclic ring containing 1, 2, or 3 heteroatoms selected from nitrogen and oxygen;
a 7-8 membered spirocyclic or fused bicyclic saturated heterocyclic ring containing 2 heteroatoms selected from nitrogen;
   -(3-7 membered monocyclic saturated heterocyclic ring containing 1 or 2 heteroatoms selected from nitrogen)-X³-(3-7 membered monocyclic saturated heterocyclic ring containing 1 or 2 heteroatoms selected from nitrogen)-, wherein X³ is C₁₋₁₀ alkylene, —O—, —N(H)—, —N(C₁₋₄ alkyl)-, or a bond;
   -(piperidinylene)-(C₁₋₅ alkylene)-(piperazinylene)-***, wherein *** is the point of attachment to A²;
   -(piperazinylene)-(azetidinylene)-*** or (azetidinylene)-(piperazinylene)-***, wherein *** is the point of attachment to A²;
   -(piperidinylene)-X³-(azetidinylene)- or -(azetidinylene)-X³-(azetidinylene)-, wherein X³ is C₁₋₃ alkylene, and *** is the point of attachment to A²;
   -(3-7 membered monocyclic saturated heterocyclic ring containing 1 or 2 heteroatoms selected from nitrogen)-X³—(C₃₋₆ cycloalkylene)-O—***, -(3-7 membered monocyclic saturated heterocyclic ring containing 1 or 2 heteroatoms selected from nitrogen)-X³—(C₃₋₆ cycloalkylene)-N(H)—***, or -(3-7 membered monocyclic saturated heterocyclic ring containing 1 or 2 heteroatoms selected from nitrogen)-X³—(C₃₋₆ cycloalkylene)-N(C₁₋₄ alkyl)-***, wherein *** is the point of attachment to A², and X³ is C₁₋₁₀ alkylene, —O—, —N(H)—, —N(C₁₋₄ alkyl)-, or a bond;
   -(piperidinylene)-X³—(C₃₋₆ cycloalkylene)-O—***, -(piperidinylene)-X³—(C₃₋₆ cycloalkylene)-N(H)—***, or -(piperidinylene)-X³—(C₃₋₆ cycloalkylene)-***, wherein *** is the point of attachment to A², and X³ is C₁₋₁₀ alkylene, —O—, —N(H)—, —N(C₁₋₄ alkyl)-, or a bond;
—N(C₁₋₃ alkyl)-(C₂₋₇ alkylene)-O—***, —N(H)—(C₂₋₇ alkylene)-O—***, —N(C₁₋₃ alkyl)-[(C₂₋₄ alkylene)-O-]₂₋₁₀-***, or —N(H)—[(C₂₋₄ alkylene)-O-]₂₋₁₀-***, where *** is a point of attachment to A²;
—N(C₁₋₃ alkyl)-(C₁₋₆ alkylene)-(5-6 membered saturated heterocyclylene containing 1 or 2 heteratoms independently selected from nitrogen and oxygen)-(C₁₋₆ alkylene)-O—*** or —N(H)—(C₁₋₆ alkylene)-(5-6 membered saturated heterocyclylene containing 1 or 2 heteratoms independently selected from nitrogen and oxygen)-(C₁₋₆ alkylene)-O—***, where *** is a point of attachment to A²; or

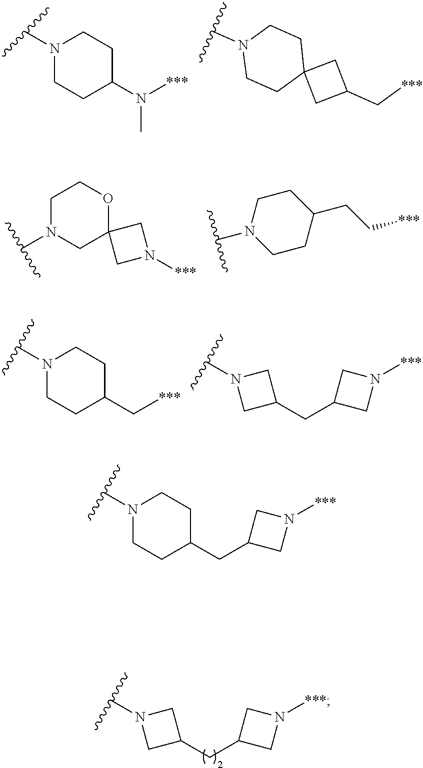

k is 1, 2, 3, or 4; and
m, n, p and t are independently 0, 1, or 2.

The definitions of variables in Formula I-la above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I-la. In certain embodiments, the definition of one or more variables set forth in Formula I-la is as set forth above in connection with Formula 1-1. Exemplary further embodiments for L are provided in Part F below.

### Part C: Compound of Formula T-1*

One aspect of the invention provides a compound represented by Formula I-1*:

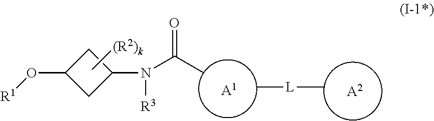

or a pharmaceutically acceptable salt thereof, wherein:
R¹ is phenyl substituted by cyano, halogen, and m occurrences of R⁴;
R² represents independently for each occurrence C₁₋₄ alkyl;
R³ is hydrogen or C₁₋₄ alkyl;
R⁴ is C₁₋₄ alkyl;
R⁵ represents independently for each occurrence C₁₋₄ alkyl or halogen, or one occurrence of R^{•} is taken together with R³ to form a C₁₋₃ alkylene;
A¹ is a pyridazinylene, pyrimidinylene, pyrazinylene, pyridinylene, phenylene, or C₅0.6 cycloalkylene, each of which is substituted with n occurrences of R⁵;
A² is one of the following:

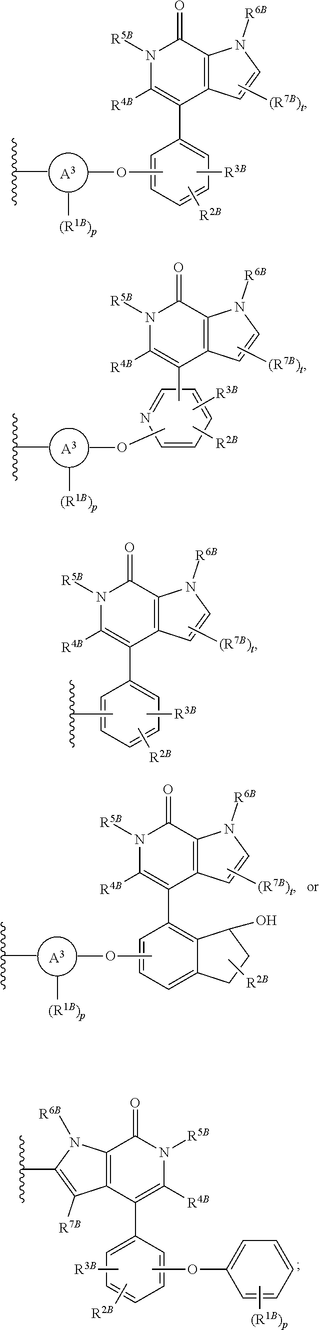

A³ is phenylene, pyridinylene, pyrazinylene, a 3-10 membered saturated monocyclic, bicyclic or spirocyclic carbocycylene, or C₁₋₆ alkylene;
R^{1B} represents independently for each occurrence halo, C₁₋₄ alkyl, or C₁₋₄ haloalkyl;
R^{2B} is hydrogen, halo, C₁₋₄ alkyl, or C₁₋₄ haloalkyl;
R^{3B} is —N(R^{8B})SO₂R^{9B}, —SO₂N(R^{8B})₂, —SO₂R^{9B}, —(C₁₋₆ alkylene)-SO₂R^{9B}, —(C₀₋₆ alkylene)-N(R^{8B})C(O)R^{9B}, —(C₀₋₆ alkylene)-C(O)N(R^{1B})₂, —N(R^{8B})₂, —NO₂, C₁₋₆ hydroxyalkyl, hydrogen, or a 4-7 membered saturated carbocyclic ring in which one CH₂ is replaced with SO₂;
R^{4B} is hydrogen, halo, or C₁₋₄ alkyl;
R^{5B} is C₁₋₄ alkyl or C₃₋₄ cycloalkyl;
R^{6B} is hydrogen, C₁₋₄ alkyl, or C₃₋₄ cycloalkyl;
R^{7B} represents independently for each occurrence C₁₋₄ alkyl or C₃₋₄ cycloalkyl;
R^{8B} represents independently for each occurrence hydrogen or C₁₋₄ alkyl; or two occurrences of R^{8B} are taken together with the nitrogen atom to which they are attached to form a 3-7 membered heterocyclic ring containing 1 or 2 heteroatoms independently selected from nitrogen and oxygen; or R^{8B} and R^{9B} are taken together with their intervening atoms to a form a 5-7 membered ring containing 1, 2, or 3 heteroatoms independently selected from nitrogen, oxygen, and sulfur;
R^{9B} is C₁₋₆ alkyl, C₁₋₆ haloalkyl, —(C₁₋₆ alkylene)-(C₃₋₆ cycloalkyl), or C₃₋₆ cycloalkyl; and
L is a linker;
k is 1, 2, 3, or 4; and
m, n, p and t are independently 0, 1, or 2.

The definitions of variables in Formula I-1* above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I-1*.

As define generally above, R¹ is phenyl substituted by cyano, halogen, and m occurrences of R⁴. In certain embodiments, R¹ is phenyl substituted by cyano, halogen, and 0 occurrences of R⁴. In certain embodiments, R¹ is phenyl substituted by cyano, halogen, and 1 occurrence of R⁴. In certain embodiments, R¹ is phenyl substituted by cyano, halogen, and 2 occurrences of R⁴. In certain embodiments, R¹ is phenyl substituted by cyano, Cl, and 0 occurrences of R⁴. In certain embodiments, R¹ is

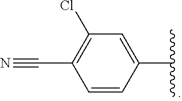 [SMILES: *c1ccc(C#N)c(Cl)c1]

In certain embodiments, R¹ is selected from the groups depicted in the compounds in Table 1 below.

As defined generally above, R² represents independently for each occurrence C₁₋₄ alkyl. In certain embodiments, R² is C₁₋₂ alkyl. In certain embodiments, R² is methyl. In certain embodiments, R² is selected from the groups depicted in the compounds in Table 1 below.

As defined generally above, R³ is hydrogen or C₁₋₄ alkyl. In certain embodiments, R³ is hydrogen. In certain embodiments, R³ is C₁₋₄ alkyl. In certain embodiments, R³ is selected from the groups depicted in the compounds in Table 1 below.

As defined generally above, R⁴ is C₁₋₄ alkyl. In certain embodiments, R⁴ is methyl. In certain embodiments, R⁴ is selected from the groups depicted in the compounds in Table 1 below.

As defined generally above, R⁵ represents independently for each occurrence C₁₋₄ alkyl or halogen, or one occurrence of R⁵ is taken together with R³ to form a C₁₋₃ alkylene. In certain embodiments, R⁵ represents independently for each occurrence C₁₋₄ alkyl. In certain embodiments, R⁵ represents independently for each occurrence C₁₋₄ alkyl or halogen. In certain embodiments, R⁵ represents independently for each occurrence methyl. In certain embodiments, R⁵ represents independently for each occurrence halogen. In certain embodiments, R⁵ represents independently for each occurrence Cl, Br, or F.

In certain embodiments, R⁵ is C₁₋₄ alkyl. In certain embodiments, R⁵ is methyl. In certain embodiments, R⁵ is halogen. In certain embodiments, R⁵ is Cl, Br, or F. In certain embodiments, one occurrence of R⁵ is taken together with R³ to form a C₁₋₃ alkylene. In certain embodiments, R⁵ is selected from the groups depicted in the compounds in Table 1 below.

As defined generally above, A¹ is a pyridazinylene, pyrimidinylene, pyrazinylene, pyridinylene, phenylene, or C₅₋₆ alkylene, each of which is substituted with n occurrences of R⁵. In certain embodiments, A¹ is a pyridazinylene, pyrimidinylene, pyrazinylene, pyridinylene, or phenylene, each of which is substituted with n occurrences of R⁵. In certain embodiments, A¹ is pyridazinylene substituted with n occurrences of R⁵. In certain embodiments, A¹ is

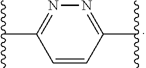 [SMILES: *c1ccc(*)nn1]

In certain embodiments, A¹ is pyrimidinylene substituted with n occurrences of R⁵. In certain embodiments, A¹ is

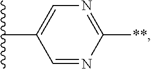

where ** is the point of attachment to L. In certain embodiments, A¹ is

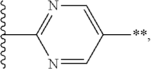

where ** is the point of attachment to L. In certain embodiments, A¹ is pyrazinylene substituted with n occurrences of R⁵. In certain embodiments, A¹ is

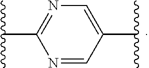 [SMILES: *c1cnc(*)nc1]

In certain embodiments, A¹ is pyridinylene substituted with n occurrences of R⁵. In certain embodiments, A¹ is

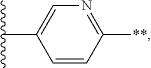

where ** is the point of attachment to L. In certain embodiments, A¹ is phenylene substituted with n occurrences of R⁵. In certain embodiments, A¹ is

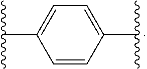 [SMILES: *c1ccc(*)cc1]

In certain embodiments, A¹ is C₅₋₆ cycloalkylene substituted with n occurrences of R⁵. In certain embodiments, A¹ is C₆ cycloalkylene substituted with n occurrences of R⁵. In certain embodiments, A¹ is a pyridazinylene substituted with 0 occurrences of R⁵. In certain embodiments, A¹ is a pyrimidinylene substituted with 0 occurrences of R⁵. In certain embodiments, A¹ is a pyrazinylene substituted with 0 occurrences of R⁵. In certain embodiments, A¹ is a pyridinylene substituted with 0 occurrences of R⁵. In certain embodiments, A¹ is a phenylene substituted with 0 occurrences of R⁵. In certain embodiments, A¹ is C₅₋₁₅. cycloalkylene substituted with 0 occurrences of R⁵. In certain embodiments, A¹ is C₆ cycloalkylene substituted with 0 occurrences of R⁵. In certain embodiments, A¹ is selected from the groups depicted in the compounds in Table 1 below.

As defined generally above, A² is

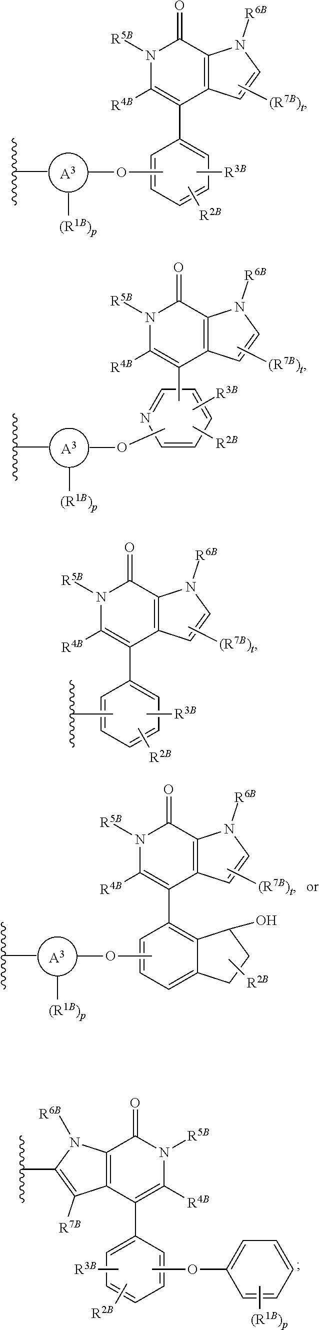

In certain embodiments, A² is

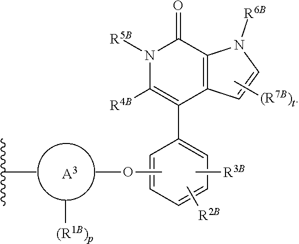

In certain embodiments, A² is

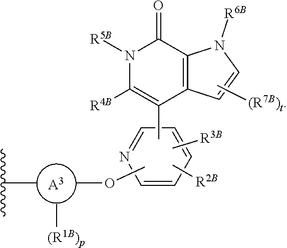

In certain embodiments, A² is

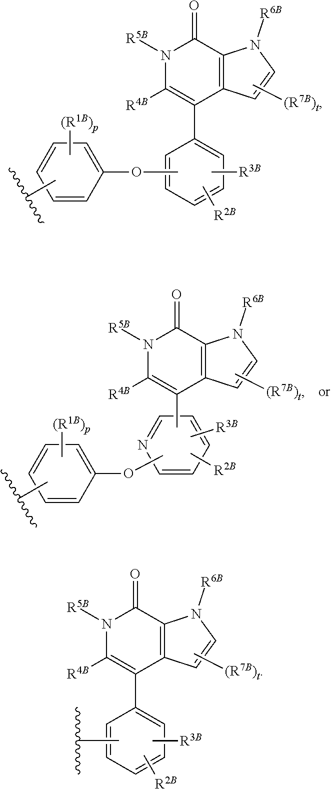

In certain embodiments, A² is

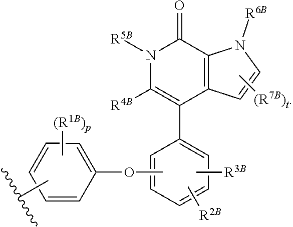

In certain embodiments, A² is

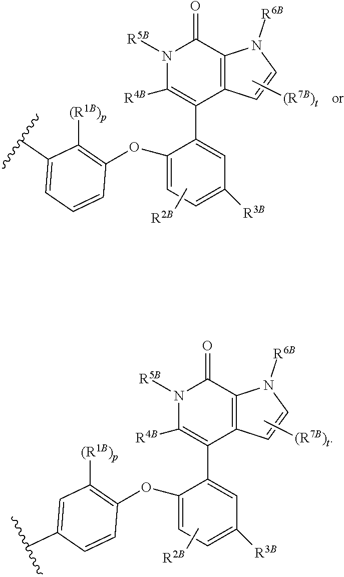

In certain embodiments, A² is

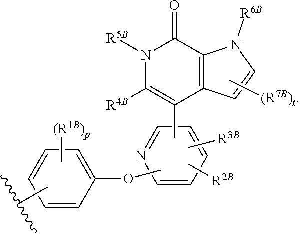

In certain embodiments, A² is

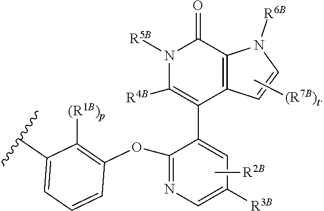

In certain embodiments, A² is

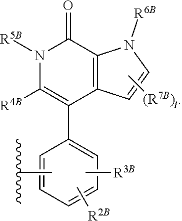

In certain embodiments, A² is

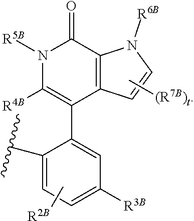

In certain embodiments, A² is

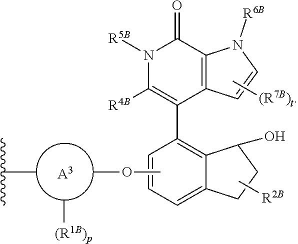

In certain embodiments, A² is

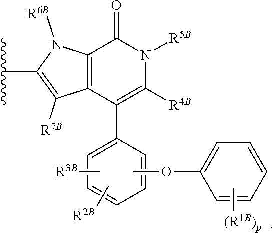

In certain embodiments, A² is

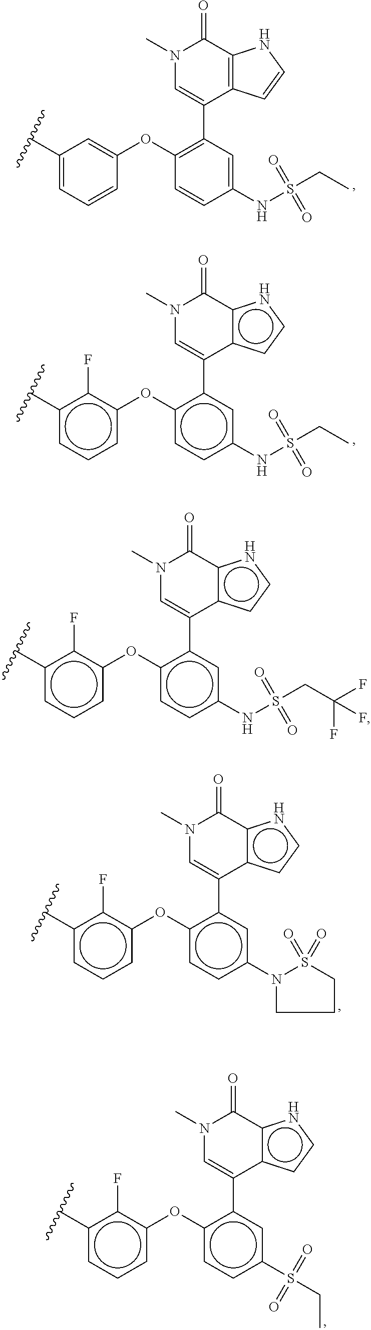

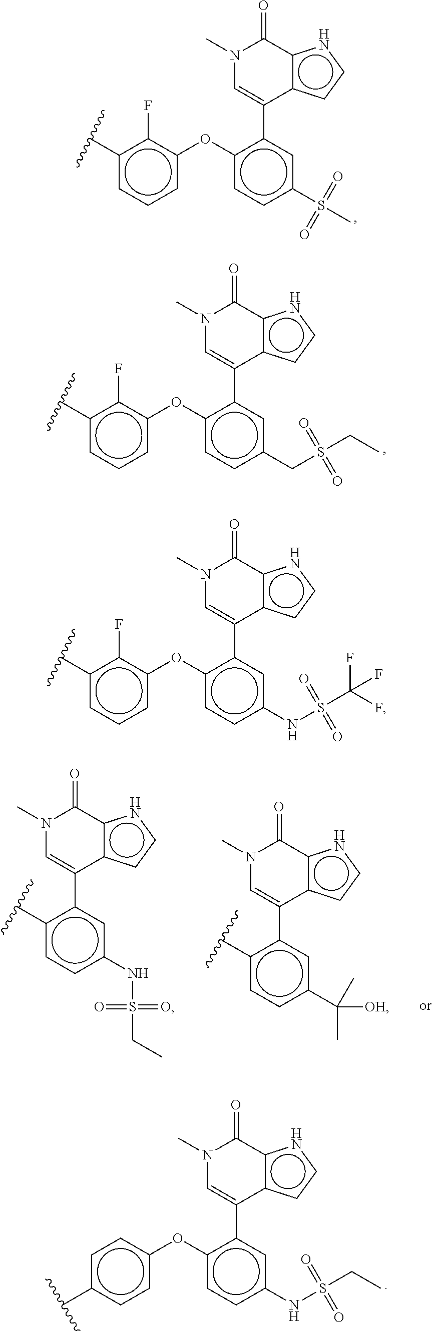

In certain embodiments, A² is

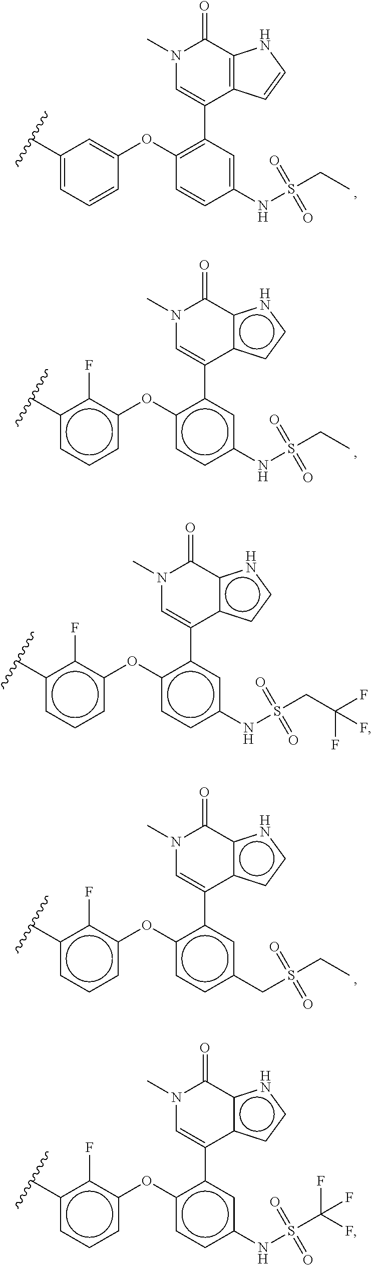

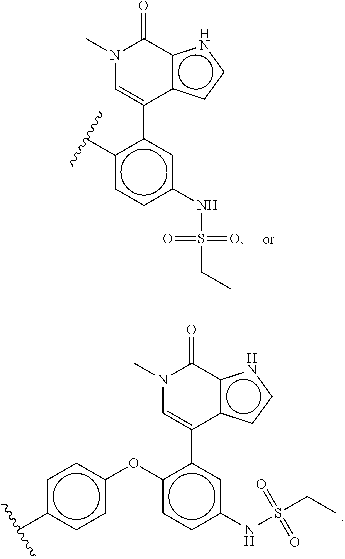

In certain embodiments, A² is

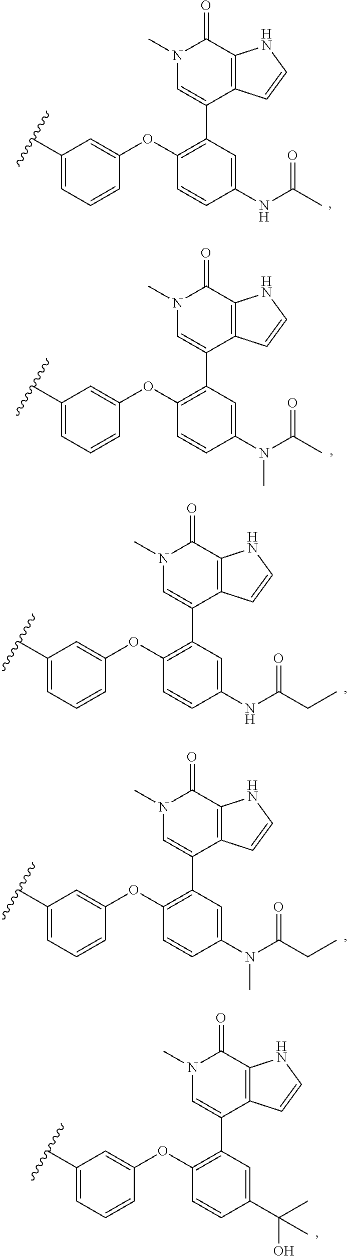

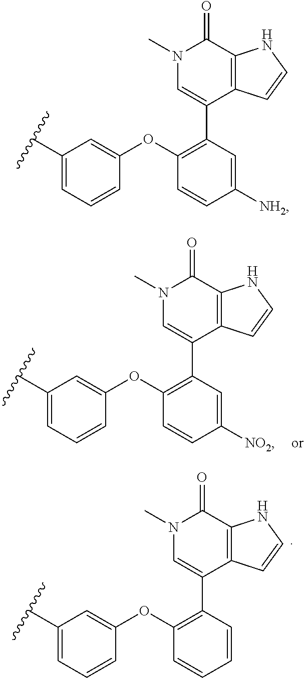

In certain embodiments, A² is

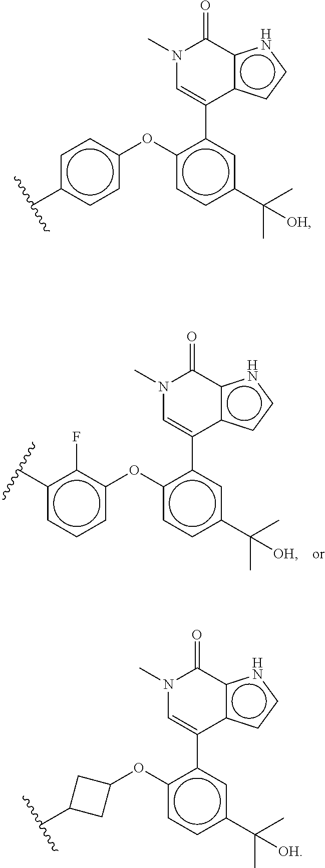

In certain embodiments, A² is

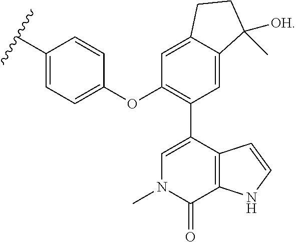 [SMILES: *c1ccc(Oc2cc3c(cc2-c2cn(C)c(=O)c4[nH]ccc24)C(C)(O)CC3)cc1]

In certain embodiments, A² is

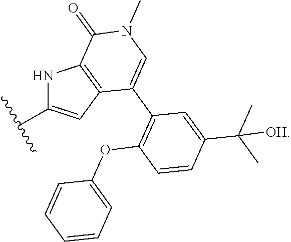 [SMILES: *c1cc2c(-c3cc(C(C)(C)O)ccc3Oc3ccccc3)cn(C)c(=O)c2[nH]1]

In certain embodiments, A² is selected from the groups depicted in the compounds in Table 1 below.

As defined generally above, A³ is phenylene, pyridinylene, pyrazinylene, a 3-10 membered saturated monocyclic, bicyclic or spirocyclic carbocycylene, or C₁₋₆ alkylene. In certain embodiments, A³ is phenylene, a 3-10 membered saturated monocyclic, bicyclic or spirocyclic carbocycylene, or C₁₋₆ alkylene. In certain embodiments, A³ is phenylene. In certain embodiments, A³ is pyridinylene. In certain embodiments, A³ is pyrazinylene. In certain embodiments, A³ is a 3-10 membered saturated monocyclic, bicyclic or spirocyclic carbocycylene. In certain embodiments, A³ is C₁₋₆ alkylene. In certain embodiments, A³ is methylene or ethylene. In certain embodiments, A³ is a 3-6 membered saturated monocyclic carbocycylene. In certain embodiments, A³ is a 6-10 membered saturated bicyclic or spirocyclic carbocycylene. In certain embodiments, A³ is selected from the groups depicted in the compounds in Table 1 below.

As defined generally above, RB represents independently for each occurrence halo, C₁₋₄ alkyl, or C₁₋₄ haloalkyl. In certain embodiments, RB is halo. In certain embodiments, RB is fluoro. In certain embodiments, R^{1B} is C₁₋₄ alkyl. In certain embodiments, R^{1B} is C₁₋₄ haloalkyl. In certain embodiments, R^{1B} is selected from the groups depicted in the compounds in Table 1 below.

As defined generally above, R^{2B} is hydrogen, halo, C₁₋₄ alkyl, or C₁₋₄ haloalkyl. In certain embodiments, R^{2B} is hydrogen. In certain embodiments, R^{2B} is halo. In certain embodiments, R^{2B} is C₁₋₄ alkyl. In certain embodiments, R^{2B} is C₁₋₄ haloalkyl. In certain embodiments, R^{2B} is selected from the groups depicted in the compounds in Table 1 below.

As defined generally above, R^{3B} is —N(R^{8B})SO₂R^{9B}, —SO₂N(R^{8B})₂, —SO₂R^{9B}, —(C₁₋₆ alkylene)-SO₂R^{9B}, —(C₀₋₆ alkylene)-N(R^{8B})C(O)R^{9B}, —(C₀₋₆ alkylene)-C(O)N(R^{8B})₂, —N(R^{8B})₂, —NO₂, C₁₋₆ hydroxyalkyl, hydrogen, or a 4-7 membered saturated carbocyclic ring in which one CH₂ is replaced with SO₂. In certain embodiments, R^{3B} is —N(R^{8B})SO₂R^{9B}. In certain embodiments, R^{3B} is —SO₂R^{9B}. In certain embodiments, R^{3B} is —(C₁₋₆ alkylene)-SO₂R^{9B}. In certain embodiments, R^{3B} is —SO₂N(R^{8B})₂. In certain embodiments, R^{3B} is —(C₀₋₆ alkylene)-N(R^{8B})C(O)R^{9B}. In certain embodiments, R^{3B} is —N(R^{8B})C(O)R^{9B}. In certain embodiments, R^{3B} is —N(H)C(O)CH₃. In certain embodiments, R^{3B} is —N(CH₃)C(O)CH₃. In certain embodiments, R^{3B} is —(C₀₋₆ alkylene)-C(O)N(R^{8B})₂. In certain embodiments, R^{3B} is —C(O)N(R^{8B})₂. In certain embodiments, R^{3B} is —N(R^{8B})₂. In certain embodiments, R^{3B} is —NH₂. In certain embodiments, R^{3B} is —NO₂. In certain embodiments, R^{3B} is C₁₋₆ hydroxyalkyl. In certain embodiments, R^{3B} is —C(CH₃)₂(OH). In certain embodiments, R^{3B} is hydrogen. In certain embodiments, R^{3B} is a 4-7 membered saturated carbocyclic ring in which one CH₂ is replaced with SO₂. In certain embodiments, R^{3B} is selected from the groups depicted in the compounds in Table 1 below.

As defined generally above, R^{4B} is hydrogen, halo, or C₁₋₄ alkyl. In certain embodiments, R^{4B} is hydrogen. In certain embodiments, R^{4B} is halo. In certain embodiments, R^{4B} is C₁₋₄ alkyl. In certain embodiments, R^{4B} is selected from the groups depicted in the compounds in Table 1 below.

As defined generally above, R^{5B} is C₁₋₄ alkyl or C₃₋₄ cycloalkyl. In certain embodiments, R^{5B} is C₁₋₄ alkyl. In certain embodiments, R^{8B} is methyl. In certain embodiments, R^{5B} is C₃₋₄ cycloalkyl. In certain embodiments, R^{3B} is selected from the groups depicted in the compounds in Table 1 below.

As defined generally above, R^{6B} is hydrogen, C₁₋₄ alkyl, or C₃₋₄ cycloalkyl. In certain embodiments, R^{6B} is hydrogen. In certain embodiments, R^{6B} is C₁₋₄ alkyl. In certain embodiments, R^{6B} is C₃₋₄ cycloalkyl. In certain embodiments, R^{6B} is selected from the groups depicted in the compounds in Table 1 below.

As defined generally above, R^{7B} represents independently for each occurrence C₁₋₄ alkyl or C₃₋₄ cycloalkyl. In certain embodiments, R^{7B} is C₁₋₄ alkyl. In certain embodiments, R^{7B} is methyl. In certain embodiments, R^{7B} is C₃₋₄ cycloalkyl. In certain embodiments, R^{7B} is selected from the groups depicted in the compounds in Table 1 below.

As defined generally above, R^{8B} represents independently for each occurrence hydrogen or C₁₋₄ alkyl; or two occurrences of R^{8B} are taken together with the nitrogen atom to which they are attached to form a 3-7 membered heterocyclic ring containing 1 or 2 heteroatoms independently selected from nitrogen and oxygen; or R^{8B} and R^{9B} are taken together with their intervening atoms to a form a 5-7 membered ring containing 1, 2, or 3 heteroatoms independently selected from nitrogen, oxygen, and sulfur. In certain embodiments, R^{8B} represents independently for each occurrence hydrogen or C₁₋₄ alkyl. In certain embodiments, R^{8B} is hydrogen. In certain embodiments, R^{8B} is C₁₋₄ alkyl. In certain embodiments, two occurrences of R^{8B} are taken together with the nitrogen atom to which they are attached to form a 3-7 membered heterocyclic ring containing 1 or 2 heteroatoms independently selected from nitrogen and oxygen. In certain embodiments, R^{8B} and R^{9B} are taken together with their intervening atoms to a form a 5-7 membered ring containing 1, 2, or 3 heteroatoms independently selected from nitrogen, oxygen, and sulfur. In certain embodiments, R^{8B} is selected from the groups depicted in the compounds in Table 1 below.

As defined generally above, R^{9B} is C₁₋₆ alkyl, C₁₋₆ haloalkyl, —(C₁₋₆ alkylene)-(C₃₋₆ cycloalkyl), or C₃₋₆ cycloalkyl. In certain embodiments, R^{9B} is C₁₋₆ alkyl or C₁₋₆ haloalkyl. In certain embodiments, R^{9B} is C₁₋₆ alkyl. In certain embodiments, R^{9B} is C₁₋₆ haloalkyl. In certain embodiments, R^{9B} is —(C₁₋₆ alkylene)-(C₃₋₆ cycloalkyl). In certain embodiments, R^{9E} is C₃₋₆ cycloalkyl. In certain embodiments, R^{9B} is selected from the groups depicted in the compounds in Table 1 below.

As defined generally above, k is 1, 2, 3, or 4. In certain embodiments, k is 1. In certain embodiments, k is 2. In certain embodiments, k is 3. In certain embodiments, k is 4. In certain embodiments, k is selected from the corresponding value in the groups depicted in the compounds in Table 1 below.

As defined generally above, m, n, p and t are independently 0, 1, or 2. In certain embodiments, p is 2. In certain embodiments, p is 1. In certain embodiments, p is 0. In certain embodiments, t is 2. In certain embodiments, m is 0. In certain embodiments, m is 1. In certain embodiments, m is 2. In certain embodiments, n is 0. In certain embodiments, n is 1. In certain embodiments, n is 2. In certain embodiments, t is 0. In certain embodiments, t is 1. In certain embodiments, p is selected from the corresponding value in the groups depicted in the compounds in Table 1 below. In certain embodiments, t is selected from the corresponding value in the groups depicted in the compounds in Table 1 below. In certain embodiments, m is selected from the corresponding value in the groups depicted in the compounds in Table 1 below. In certain embodiments, n is selected from the corresponding value in the groups depicted in the compounds in Table 1 below.

In certain embodiments, the compound of Formula I-1* is further defined by Formula Ia or a pharmaceutically acceptable salt thereof.

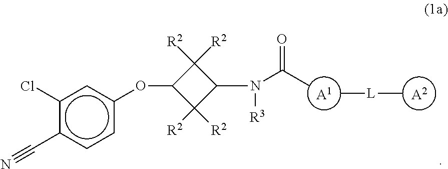

In certain embodiments, the definition of variables R², R³, A¹, and A² is one of the embodiments described above in connection with Formula I-1*.

In certain embodiments, the compound of Formula 1-1*is further defined by Formula Ib or a pharmaceutically acceptable salt thereof:

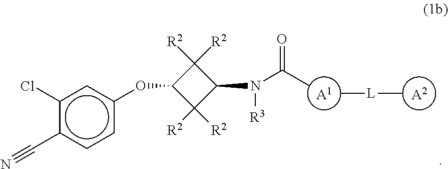

In certain embodiments, the definition of variables R², R³, A¹, and A² is one of the embodiments described above in connection with Formula I-1*.

In certain embodiments, the compound of Formula I-1* is further defined by Formula Ic or a pharmaceutically acceptable salt thereof:

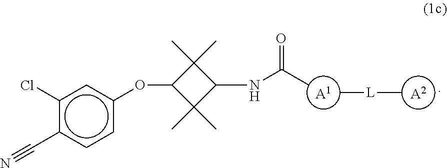

In certain embodiments, the definition of variables A¹ and A² is one of the embodiments described above in connection with Formula I-1*.

In certain embodiments, the compound of Formula I-1* is further defined by Formula Id or a pharmaceutically acceptable salt thereof:

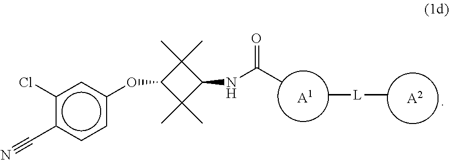

In certain embodiments, the definition of variables A¹ and A² is one of the embodiments described above in connection with Formula I-1*.

The compounds may be further characterized according to, for example, the identity of L. Exemplary further embodiments for L are provided in Part F below.

Another aspect of the invention provides a compound represented by Formula I-la*:

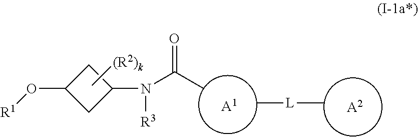

or a pharmaceutically acceptable salt thereof, wherein:
R¹ is phenyl substituted by cyano, halogen, and m occurrences of R⁴;
R² represents independently for each occurrence C₁₋₄ alkyl;
R³ is hydrogen or C₁₋₄ alkyl;
R⁴ is C₁₋₄ alkyl;
R⁵ represents independently for each occurrence C₁₋₄ alkyl or halogen, or one occurrence of R⁵ is taken together with R³ to form a C₁₋₃ alkylene;
A¹ is a pyridazinylene, pyrimidinylene, pyrazinylene, pyridinylene, phenylene, or C₅₋₆ cycloalkylene, each of which is substituted with n occurrences of R⁵;
A² is one of the following:

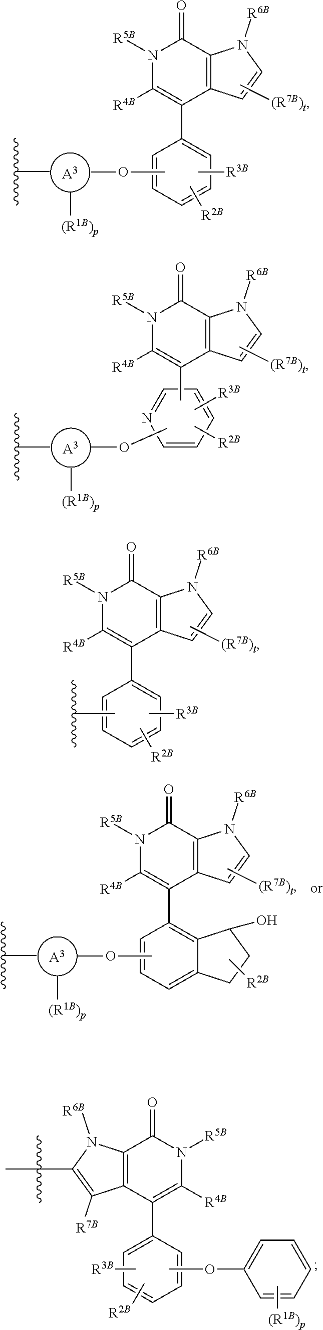

A³ is phenylene, pyridinylene, pyrazinylene, a 3-10 membered saturated monocyclic, bicyclic or spirocyclic carbocycylene, or C₁₋₆ alkylene;
R^{1B} represents independently for each occurrence halo, C₁₋₄ alkyl, or C₁₋₄ haloalkyl;
R^{2B} is hydrogen, halo, C₁₋₄ alkyl, or C₁₋₄ haloalkyl;
R^{3B} is —N(R^{8B})SO₂R^{9B}, —SO₂N(R^{8B})₂, —SO₂R^{9B}, —(C₁₋₆ alkylene)-SO₂R^{9B}, —(C₀₋₆ alkylene)-N(R^{8B})C(O)R^{9B}, —(C₀₋₆ alkylene)-C(O)N(R^{8B})₂, —N(R^{8B})₂, —NO₂, C₁₋₆ hydroxyalkyl, hydrogen, or a 4-7 membered saturated carbocyclic ring in which one CH₂ is replaced with SO₂;
R^{4B} is hydrogen, halo, or C₁₋₄ alkyl;
R^{5B} is C₁₋₄ alkyl or C₃₋₄ cycloalkyl;
R^{6B} is hydrogen, C₁₋₄ alkyl, or C₃₋₄ cycloalkyl;
R^{1B} represents independently for each occurrence C₁₋₄ alkyl or C₃₋₄ cycloalkyl;
R^{8B} represents independently for each occurrence hydrogen or C₁₋₄ alkyl; or two occurrences of R^{8B} are taken together with the nitrogen atom to which they are attached to form a 3-7 membered heterocyclic ring containing 1 or 2 heteroatoms independently selected from nitrogen and oxygen; or R^{8B} and R^{9B} are taken together with their intervening atoms to a form a 5-7 membered ring containing 1, 2, or 3 heteroatoms independently selected from nitrogen, oxygen, and sulfur;
R^{9B} is C₁₋₆ alkyl, C₁₋₆ haloalkyl, —(C₁₋₆ alkylene)-(C₃₋₆ cycloalkyl), or C₃₋₆ cycloalkyl; and
L is one of the following:
   a 7-11 membered spirocyclic or fused bicyclic saturated heterocyclic ring containing 1, 2, or 3 heteroatoms selected from nitrogen and oxygen;
   a 7-8 membered spirocyclic or fused bicyclic saturated heterocyclic ring containing 2 heteroatoms selected from nitrogen;
   -(3-7 membered monocyclic saturated heterocyclic ring containing 1 or 2 heteroatoms selected from nitrogen)-X³-(3-7 membered monocyclic saturated heterocyclic ring containing 1 or 2 heteroatoms selected from nitrogen)-, wherein X³ is C₁₋₁₀ alkylene, —O—, —N(H)—, —N(C₁₋₄ alkyl)-, or a bond;
   -(piperidinylene)-(C₁₋₅ alkylene)-(piperazinylene)-***, wherein *** is the point of attachment to A²;
   -(piperazinylene)-(azetidinylene)-*** or (azetidinylene)-(piperazinylene)-***, wherein *** is the point of attachment to A²;
   -(piperidinylene)-X³-(azetidinylene)- or -(azetidinylene)-X³-(azetidinylene)-, wherein X³ is C₁₋₃ alkylene, and *** is the point of attachment to A²;
   -(3-7 membered monocyclic saturated heterocyclic ring containing 1 or 2 heteroatoms selected from nitrogen)-X³—(C₃₋₆ cycloalkylene)-O—***, -(3-7 membered monocyclic saturated heterocyclic ring containing 1 or 2 heteroatoms selected from nitrogen)-X³—(C₃₋₆ cycloalkylene)-N(H)—***, or -(3-7 membered monocyclic saturated heterocyclic ring containing 1 or 2 heteroatoms selected from nitrogen)-X³—(C₃₋₆ cycloalkylene)-N(C₁₋₄ alkyl)-***, wherein *** is the point of attachment to A², and X³ is C₁₋₁₀ alkylene, —O—, —N(H)—, —N(C₁₋₄ alkyl)-, or a bond;
   -(piperidinylene)-X³—(C₃₋₆ cycloalkylene)-O—***, -(piperidinylene)-X³—(C₃₋₆ cycloalkylene)-N(H)—***, or -(piperidinylene)-X³—(C₃₋₆ cycloalkylene)-***, wherein *** is the point of attachment to A², and X³ is C₁₋₁₀ alkylene, —O—, —N(H)—, —N(C₁₋₄ alkyl)-, or a bond;
—N(C₁₋₃ alkyl)-(C₂₋₇ alkylene)-O—***, —N(H)—(C₂₋₇ alkylene)-O—***, —N(C₁₋₃ alkyl)-[(C₂₋₄ alkylene)-O-]₂₋₁₀-***, or —N(H)—[(C₂₋₄ alkylene)-O-]₂₋₁₀-***, where *** is a point of attachment to A²;
—N(C₁₋₃ alkyl)-(C₁₋₆ alkylene)-(5-6 membered saturated heterocyclylene containing 1 or 2 heteratoms independently selected from nitrogen and oxygen)-(C₁₋₆ alkylene)-O—*** or —N(H)—(C₁₋₆ alkylene)-(5-6 membered saturated heterocyclylene containing 1 or 2 heteratoms independently selected from nitrogen and oxygen)-(C₁₋₆ alkylene)-O—***, where *** is a point of attachment to A²; or

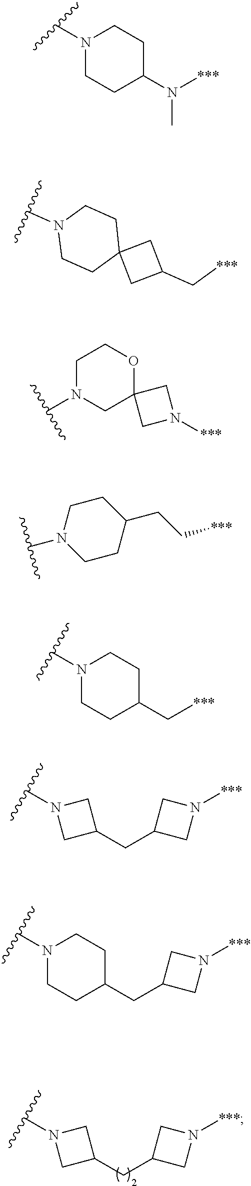

k is 1, 2, 3, or 4; and
m, n, p and t are independently 0, 1, or 2.

The definitions of variables in Formula I-la* above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I-la*. In certain embodiments, the definition of one or more variables set forth in Formula I-la* is as set forth above in connection with Formula T-1. Exemplary further embodiments for L are provided in Part F below.

### Part D: Compound of Formula II

Another aspect of the invention provides a compound represented by Formula II:

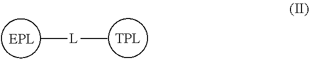

or a pharmaceutically acceptable salt thereof; wherein:
TPL is a group defined by Formula II-1 that is substituted by one occurrence of R^{1I}-A wherein Formula II-1 is represented by:

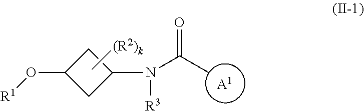

or a pharmaceutically acceptable salt thereof, wherein:
R^{II-1A} is a bond to L;
R¹ is phenyl substituted by cyano, halogen, and m occurrences of R⁴;
R² represents independently for each occurrence C₁₋₄ alkyl;
R³ is hydrogen or C₁₋₄ alkyl;
R⁴ is C₁₋₄ alkyl;
R⁵ represents independently for each occurrence C₁₋₄ alkyl or halogen;
A¹ is a pyridazinyl, pyrimidinyl, pyrazinyl, or pyridinyl, each of which is substituted with n occurrences of R⁵;
L is a linker;
EPL is a moiety that binds to BRD4;
k is 1, 2, 3, or 4; and
m and n are independently 0, 1, or 2.

The definitions of variables in Formula II above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula II.

As defined generally above, R¹ is phenyl substituted by cyano, halogen, and m occurrences of R⁴. In certain embodiments, R¹ is phenyl substituted by cyano, halogen, and 0 occurrences of R⁴. In certain embodiments, R¹ is

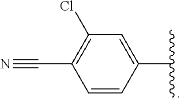 [SMILES: *c1ccc(C#N)c(Cl)c1]

In certain embodiments, R¹ is phenyl substituted by cyano, halogen, and 1 occurrences of R⁴. In certain embodiments, R¹ is phenyl substituted by cyano, halogen, and 2 occurrences of R⁴. In certain embodiments, R¹ is phenyl substituted by cyano, Cl, and 0 occurrences of R⁴. In certain embodiments, R^{•} is selected from the groups depicted in the compounds in Table 1 below.

As defined generally above, R² represents independently for each occurrence C₁₋₄ alkyl. In certain embodiments, R² is C₁₋₁₂ alkyl. In certain embodiments, R² is methyl. In certain embodiments, R² is selected from the groups depicted in the compounds in Table 1 below.

As defined generally above, R³ is hydrogen or C₁₋₄ alkyl. In certain embodiments, R³ is hydrogen. In certain embodiments, R³ is C₁₋₄ alkyl. In certain embodiments, R³ is selected from the groups depicted in the compounds in Table 1 below.

As defined generally above, R⁴ is C₁₋₄ alkyl. In certain embodiments, R⁴ is methyl. In certain embodiments, R⁴ is selected from the groups depicted in the compounds in Table 1 below.

As defined generally above, R⁵ represents independently for each occurrence C₁₋₄ alkyl or halogen. In certain embodiments, R⁵ represents independently for each occurrence C₁₋₄ alkyl. In certain embodiments, R⁵ represents independently for each occurrence methyl. In certain embodiments, R⁵ represents independently for each occurrence halogen. In certain embodiments, R⁵ represents independently for each occurrence Cl, Br, or F.

In certain embodiments, R⁵ is C₁₋₄ alkyl. In certain embodiments, R⁵ is methyl. In certain embodiments, R⁵ is halogen. In certain embodiments, R⁵ is Cl, Br, or F. In certain embodiments, R⁵ is selected from the groups depicted in the compounds in Table 1 below.

As defined generally above, A¹ is a pyridazinyl, pyrimidinyl, pyrazinyl, or pyridinyl, each of which is substituted with n occurrences of R⁵. In certain embodiments, A¹ is pyridazinyl substituted with n occurrences of R⁵. In certain embodiments, A¹ is pyrimidinyl substituted with n occurrences of R⁵. In certain embodiments, A¹ is pyrazinyl substituted with n occurrences of R⁵. In certain embodiments, A¹ is pyridinyl substituted with n occurrences of R⁵.

As defined generally above, k is 1, 2, 3, or 4. In certain embodiments, k is 1. In certain embodiments, k is 2. In certain embodiments, k is 3. In certain embodiments, k is 4. In certain embodiments, k is selected from the corresponding value in the groups depicted in the compounds in Table 1 below.

In certain embodiments, m is 0. In certain embodiments, m is 1. In certain embodiments, m is 2. In certain embodiments, n is 0. In certain embodiments, n is 1. In certain embodiments, n is 2. In certain embodiments, m is selected from the corresponding value in the groups depicted in the compounds in Table 1 below. In certain embodiments, n is selected from the corresponding value in the groups depicted in the compounds in Table 1 below.

In certain embodiments, the TPL is

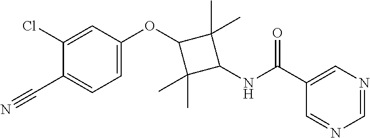 [SMILES: CC1(C)C(NC(=O)c2cncnc2)C(C)(C)C1Oc1ccc(C#N)c(Cl)c1]

that is substituted by one occurrence of R^{II-1A}.

In certain embodiments, the TPL is

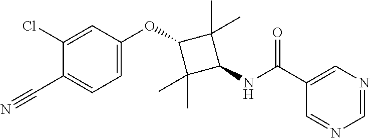 [SMILES: CC1(C)[C@H](NC(=O)c2cncnc2)C(C)(C)[C@H]1Oc1ccc(C#N)c(Cl)c1]

that is substituted by one occurrence of R^{II-1A} In certain embodiments, the TPL is

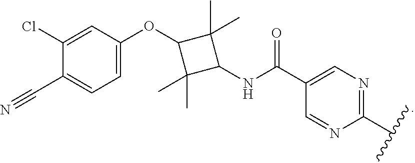 [SMILES: *c1ncc(C(=O)NC2C(C)(C)C(Oc3ccc(C#N)c(Cl)c3)C2(C)C)cn1]

In certain embodiments, the TPL is

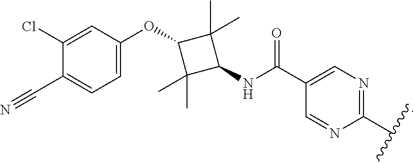 [SMILES: *c1ncc(C(=O)N[C@H]2C(C)(C)[C@H](Oc3ccc(C#N)c(Cl)c3)C2(C)C)cn1]

In certain embodiments, the EPL is defined by Formula II-2 that is substituted by one occurrence of R¹-2, wherein Formula II-2 is represented by:

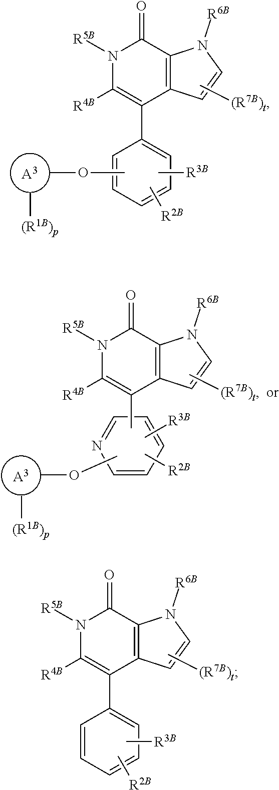

wherein
R^{II-2A} is a bond to L;
A³ is phenylene, pyridinylene, pyrazinylene, a 3-10 membered saturated monocyclic, bicyclic or spirocyclic carbocycylene, or C₁₋₆ alkylene;
R^{1B} represents independently for each occurrence halo, C₁₋₄ alkyl, or C₁₋₄ haloalkyl;
R^{2B} is hydrogen, halo, C₁₋₄ alkyl, or C₁₋₄ haloalkyl;
R^{3B} is —N(R^{8B})SO₂R^{9B}, —SO₂N(R^{8B})₂, —SO₂R^{9B}, —(C₁₋₆ alkylene)-SO₂R^{9B}, —(C₀₋₆ alkylene)-N(R^{8B})C(O)R^{9B}, —(C₀₋₆ alkylene)-C(O)N(R^{8B})₂, —N(R^{8B})₂, —NO₂, C₁₋₆ hydroxyalkyl, hydrogen, or a 4-7 membered saturated carbocyclic ring in which one CH₂ is replaced with SO₂;
R^{4B} is hydrogen, halo, or C₁₋₄ alkyl;
R^{5B} is C₁₋₄ alkyl or C₃₋₄ cycloalkyl;
R^{6B} is hydrogen, C₁₋₄ alkyl, or C₃₋₄ cycloalkyl;
R^{1B} represents independently for each occurrence C₁₋₄ alkyl or C₃₋₄ cycloalkyl;
R^{8B} represents independently for each occurrence hydrogen or C₁₋₄ alkyl; or two occurrences of R^{8B} are taken together with the nitrogen atom to which they are attached to form a 3-7 membered heterocyclic ring containing 1 or 2 heteroatoms independently selected from nitrogen and oxygen; or R^{8B} and R^{9B} are taken together with their intervening atoms to a form a 5-7 membered ring containing 1, 2, or 3 heteroatoms independently selected from nitrogen, oxygen, and sulfur;
R^{9B} is C₁₋₆ alkyl, C₁₋₆ haloalkyl, —(C₁₋₆ alkylene)-(C₃₋₆ cycloalkyl), or C₃₋₆ cycloalkyl; and p and t are independently 0, 1, or 2.

In certain embodiments, the EPL is defined by Formula II-2 that is substituted by one occurrence of R^{II-2A}, wherein Formula II-2 is represented by:

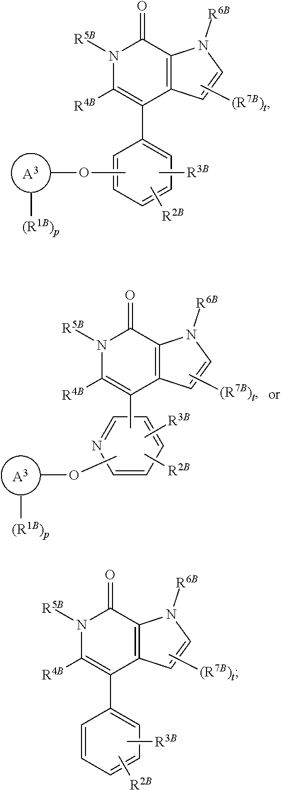

wherein
R^{II-2A} is a bond to L;
A³ is phenylene, a 3-10 membered saturated monocyclic, bicyclic or spirocyclic carbocycylene, or C₁₋₆ alkylene;
R^{1B} represents independently for each occurrence halo, C₁₋₄ alkyl, or C₁₋₄ haloalkyl;
R^{2B} is hydrogen, halo, C₁₋₄ alkyl, or C₁₋₄ haloalkyl;
R^{3B} is —N(R^{8B})SO₂R^{9B}, —SO₂N(R^{8B})₂, —SO₂R^{9B}, —(C₁₋₆ alkylene)-SO₂R^{9B}, —(C₀₋₆ alkylene)-N(R^{8B})C(O)R^{9B}, —(C₀₋₆ alkylene)-C(O)N(R^{8B})₂, —N(R^{8B})₂, —NO₂, C₁₋₆ hydroxyalkyl, hydrogen, or a 4-7 membered saturated carbocyclic ring in which one CH₂ is replaced with SO₂;
R^{4B} is hydrogen, halo, or C₁₋₄ alkyl;
R^{5B} is C₁₋₄ alkyl or C₃₋₄ cycloalkyl;
R^{6B} is hydrogen, C₁₋₄ alkyl, or C₃₋₄ cycloalkyl;
R^{1B} represents independently for each occurrence C₁₋₄ alkyl or C₃₋₄ cycloalkyl;
R^{1B} represents independently for each occurrence hydrogen or C₁₋₄ alkyl; or two occurrences of R^{8B} are taken together with the nitrogen atom to which they are attached to form a 3-7 membered heterocyclic ring containing 1 or 2 heteroatoms independently selected from nitrogen and oxygen; or R^{8B} and R^{9B} are taken together with their intervening atoms to a form a 5-7 membered ring containing 1, 2, or 3 heteroatoms independently selected from nitrogen, oxygen, and sulfur;
R^{9B} is C₁₋₆ alkyl, C₁₋₆ haloalkyl, —(C₁₋₆ alkylene)-(C₃₋₆ cycloalkyl), or C₃₋₆ cycloalkyl; and
p and t are independently 0, 1, or 2.

In certain embodiments, the EPL is

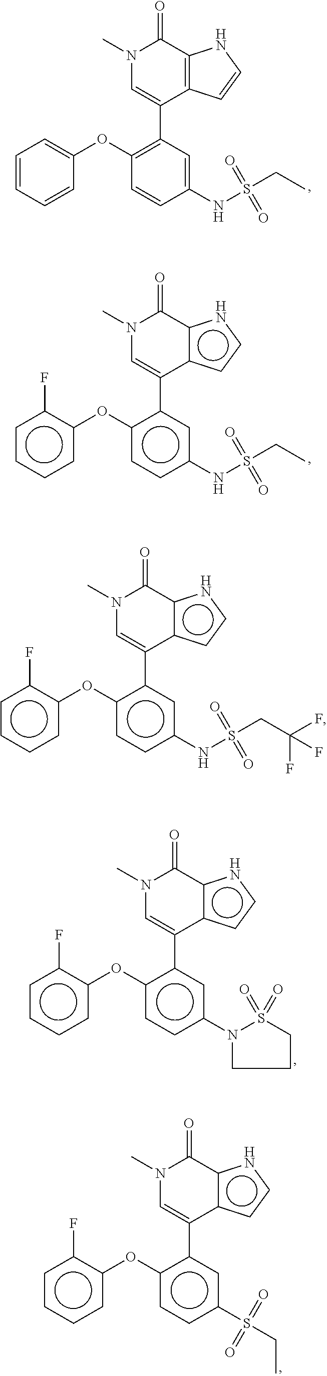

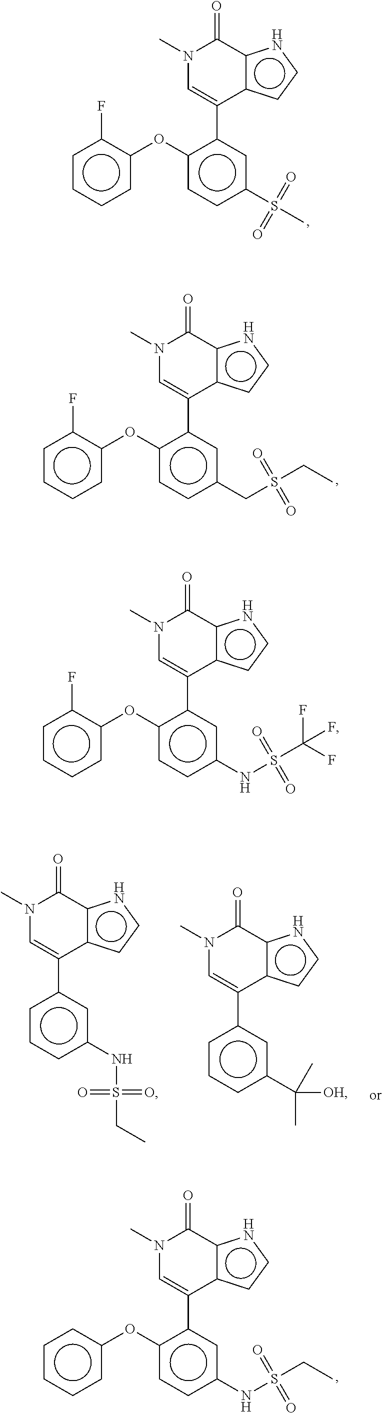

each of which is substituted by one occurrence of R^{II-2A} wherein R^{II-2A} is a bond to L.

In certain embodiments, the EPL is

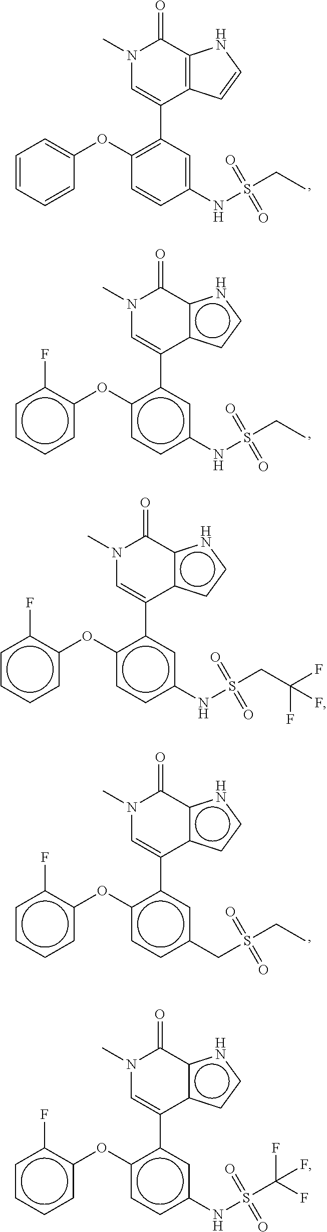

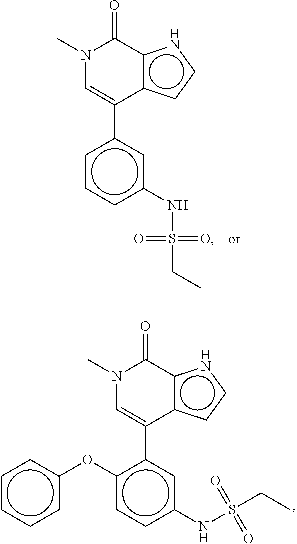

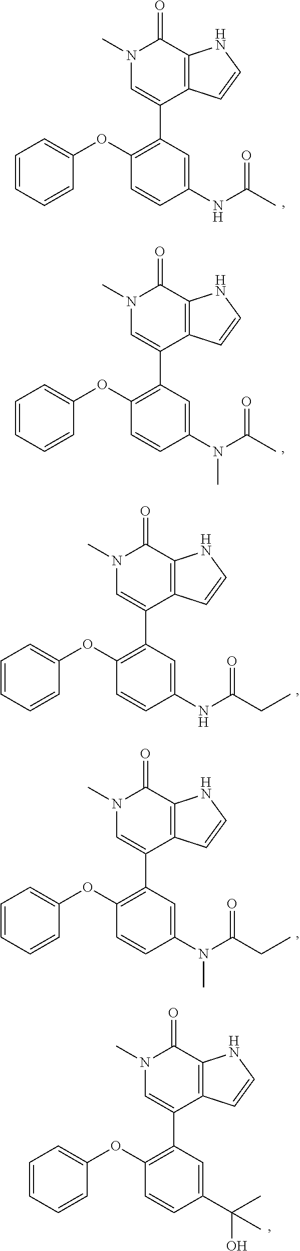

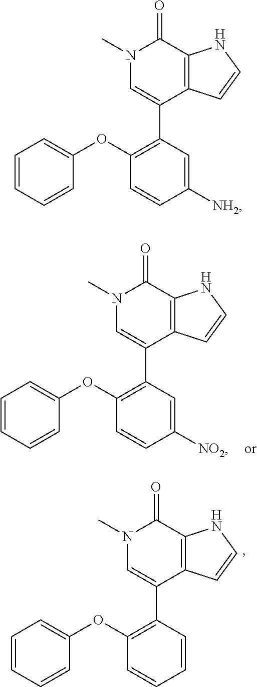

each of which is substituted by one occurrence of R^{II-2A} wherein R^{II-2A} is a bond to L.

In certain embodiments, the EPL is

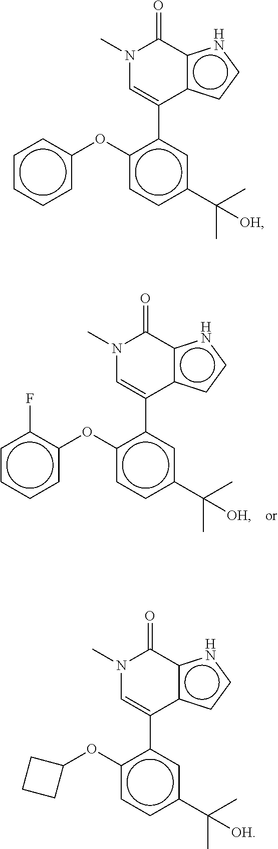

In certain embodiments, the EPL is defined by Formula II-2 that is substituted by one occurrence of R^{II-1A}, wherein Formula II-2 is represented by:

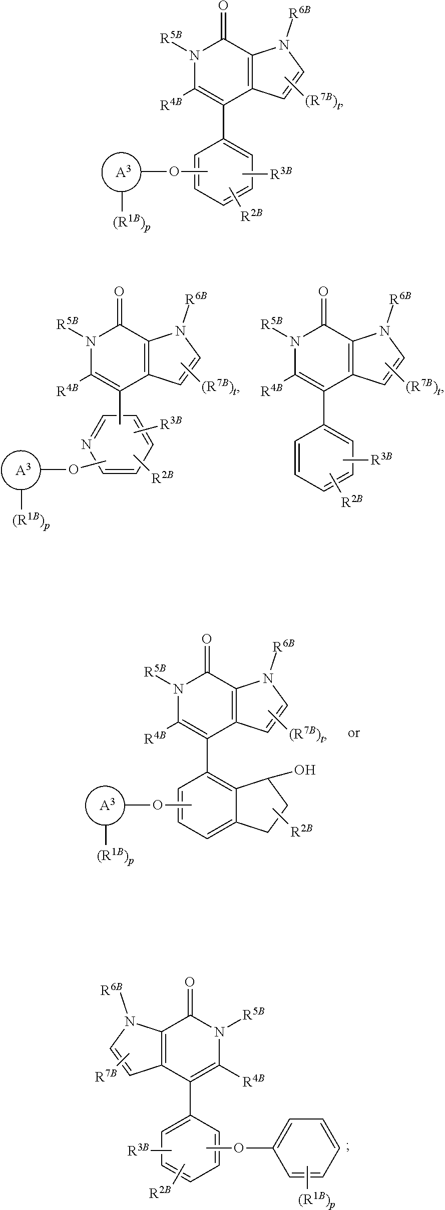

wherein
R^{II-2A} is a bond to L;
A³ is phenylene, a 3-10 membered saturated monocyclic, bicyclic or spirocyclic carbocycylene, or C₁₋₆ alkylene;
R^{1B} represents independently for each occurrence halo, C₁₋₄ alkyl, or C₁₋₄ haloalkyl;
R^{2B} is hydrogen, halo, C₁₋₄ alkyl, or C₁₋₄ haloalkyl;
R^{3B} is —N(R^{8B})SO₂R^{9B}, —SO₂N(R^{8B})₂, —SO₂R^{9B}, —(C₁₋₆ alkylene)-SO₂R^{9B}, —(C₀₋₆ alkylene)-N(R^{8B})C(O)R^{9B}, —(C₀₋₆ alkylene)-C(O)N(R^{8B})₂, —N(R^{8B})₂, —NO₂, C₁₋₆ hydroxyalkyl, hydrogen, or a 4-7 membered saturated carbocyclic ring in which one CH₂ is replaced with SO₂;
R^{4B} is hydrogen, halo, or C₁₋₄ alkyl;
R^{5B} is C₁₋₄ alkyl or C₃₋₄ cycloalkyl;
R^{6B} is hydrogen, C₁₋₄ alkyl, or C₃₋₄ cycloalkyl;
R^{7B} represents independently for each occurrence C₁₋₄ alkyl or C₃₋₄ cycloalkyl;
R^{1B} represents independently for each occurrence hydrogen or C₁₋₄ alkyl; or two occurrences of R^{8B} are taken together with the nitrogen atom to which they are attached to form a 3-7 membered heterocyclic ring containing 1 or 2 heteroatoms independently selected from nitrogen and oxygen; or R^{8B} and R^{9B} are taken together with their intervening atoms to a form a 5-7 membered ring containing 1, 2, or 3 heteroatoms independently selected from nitrogen, oxygen, and sulfur;
R^{9B} is C₁₋₆ alkyl, C₁₋₆ haloalkyl, —(C₁₋₆ alkylene)-(C₃₋₆ cycloalkyl), or C₃₋₆ cycloalkyl; and p and t are independently 0, 1, or 2.

In certain embodiments, the EPL is one of the following:

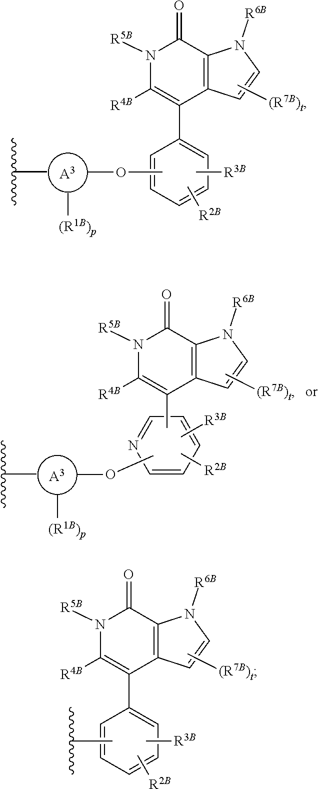

A³ is phenylene, pyridinylene, pyrazinylene, a 3-10 membered saturated monocyclic, bicyclic or spirocyclic carbocycylene, or C₁₋₆ alkylene;
R^{1B} represents independently for each occurrence halo, C₁₋₄ alkyl, or C₁₋₄ haloalkyl;
R^{2B} is hydrogen, halo, C₁₋₄ alkyl, or C₁₋₄ haloalkyl;
R^{3B} is —N(R^{8B})SO₂R^{9B}, —SO₂N(R^{8B})₂, —SO₂R^{9B}, —(C₁₋₆ alkylene)-SO₂R^{9B}, —(C₀₋₆ alkylene)-N(R^{8B})C(O)R^{9B}, —(C₀₋₆ alkylene)-C(O)N(R^{8B})₂, —N(R^{1B})₂, —NO₂, C₁₋₆ hydroxyalkyl, hydrogen, or a 4-7 membered saturated carbocyclic ring in which one CH₂ is replaced with SO₂;
R^{4B} is hydrogen, halo, or C₁₋₄ alkyl;
R^{5B} is C₁₋₄ alkyl or C₃₋₄ cycloalkyl;
R^{6B} is hydrogen, C₁₋₄ alkyl, or C₃₋₄ cycloalkyl;
R^{7B} represents independently for each occurrence C₁₋₄ alkyl or C₃₋₄ cycloalkyl;
R^{8B} represents independently for each occurrence hydrogen or C₁₋₄ alkyl; or two occurrences of R^{8B} are taken together with the nitrogen atom to which they are attached to form a 3-7 membered heterocyclic ring containing 1 or 2 heteroatoms independently selected from nitrogen and oxygen; or R^{8B} and R^{9B} are taken together with their intervening atoms to a form a 5-7 membered ring containing 1, 2, or 3 heteroatoms independently selected from nitrogen, oxygen, and sulfur;
R^{9B} is C₁₋₆ alkyl, C₁₋₆ haloalkyl, —(C₁₋₆ alkylene)-(C₃₋₆ cycloalkyl), or C₃₋₆ cycloalkyl; and
p and t are independently 0, 1, or 2.

In certain embodiments, the EPL is one of the following:

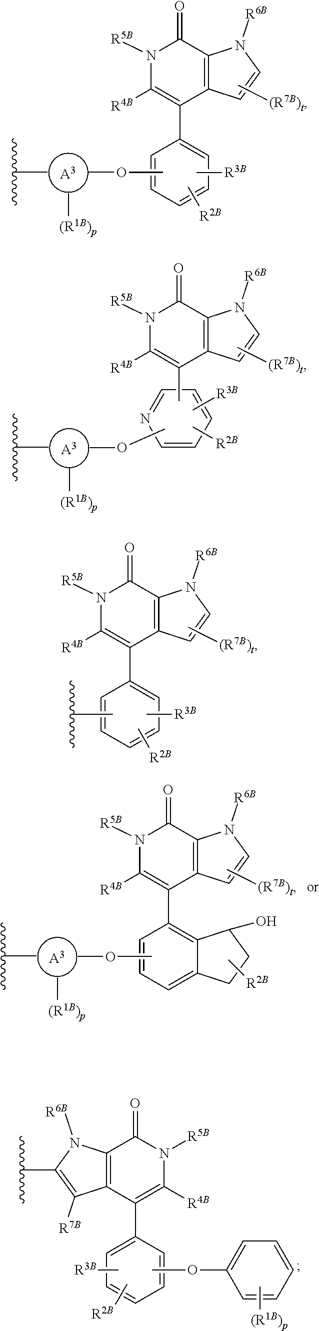

A³ is phenylene, pyridinylene, pyrazinylene, a 3-10 membered saturated monocyclic, bicyclic or spirocyclic carbocycylene, or C₁₋₆ alkylene;
R^{1B} represents independently for each occurrence halo, C₁₋₄ alkyl, or C₁₋₄ haloalkyl;
R^{2B} is hydrogen, halo, C₁₋₄ alkyl, or C₁₋₄ haloalkyl;
R^{3B} is —N(R^{8B})SO₂R^{9B}, —SO₂N(R^{8B})₂, —SO₂R^{9B}, —(C₁₋₆ alkylene)-SO₂R^{9B}, —(C₀₋₆ alkylene)-N(R^{8B})C(O)R^{9B}, —(C₀₋₆ alkylene)-C(O)N(R^{8B})₂, —N(R^{8B})₂, —NO₂, C₁₋₆ hydroxyalkyl, hydrogen, or a 4-7 membered saturated carbocyclic ring in which one CH₂ is replaced with SO₂;
R^{4B} is hydrogen, halo, or C₁₋₄ alkyl;
R^{5B} is C₁₋₄ alkyl or C₃₋₄ cycloalkyl;
R^{6B} is hydrogen, C₁₋₄ alkyl, or C₃₋₄ cycloalkyl;
R^{7B} represents independently for each occurrence C₁₋₄ alkyl or C₃₋₄ cycloalkyl;
R^{8B} represents independently for each occurrence hydrogen or C₁₋₄ alkyl; or two occurrences of R^{8B} are taken together with the nitrogen atom to which they are attached to form a 3-7 membered heterocyclic ring containing 1 or 2 heteroatoms independently selected from nitrogen and oxygen; or R^{8B} and R^{9B} are taken together with their intervening atoms to a form a 5-7 membered ring containing 1, 2, or 3 heteroatoms independently selected from nitrogen, oxygen, and sulfur;
R^{9B} is C₁₋₆ alkyl, C₁₋₆ haloalkyl, —(C₁₋₆ alkylene)-(C₃₋₆ cycloalkyl), or C₃₋₆ cycloalkyl; and
p and t are independently 0, 1, or 2.

In certain embodiments, the EPL is one of the following:

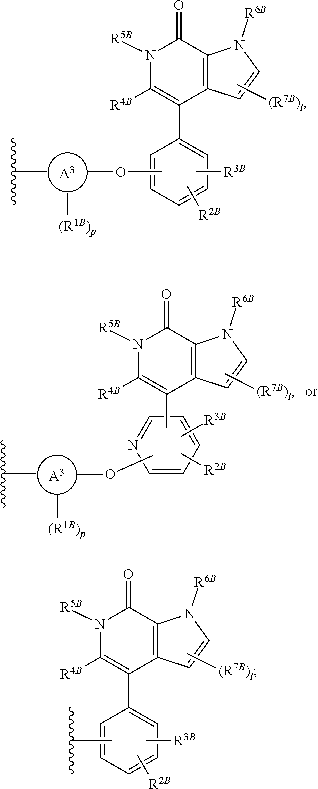

A³ is phenylene, a 3-10 membered saturated monocyclic, bicyclic or spirocyclic carbocycylene, or C₁₋₆ alkylene;
R^{1B} represents independently for each occurrence halo, C₁₋₄ alkyl, or C₁₋₄ haloalkyl;
R^{2B} is hydrogen, halo, C₁₋₄ alkyl, or C₁₋₄ haloalkyl;
R^{3B} is —N(R^{8B})SO₂R^{9B}, —SO₂N(R^{8B})₂, —SO₂R^{9B}, —(C₁₋₆ alkylene)-SO₂R^{9B}, —(C₀₋₆ alkylene)-N(R^{8B})C(O)R^{9B}, —(C₀₋₆ alkylene)-C(O)N(R^{8B})₂, —N(R^{8B})₂, —NO₂, C₁₋₆ hydroxyalkyl, hydrogen, or a 4-7 membered saturated carbocyclic ring in which one CH₂ is replaced with SO₂;
R^{4B} is hydrogen, halo, or C₁₋₄ alkyl;
R^{5B} is C₁₋₄ alkyl or C₃₋₄ cycloalkyl;
R^{6B} is hydrogen, C₁₋₄ alkyl, or C₃₋₄ cycloalkyl;
R^{1B} represents independently for each occurrence C₁₋₄ alkyl or C₃₋₄ cycloalkyl;
R^{8B} represents independently for each occurrence hydrogen or C₁₋₄ alkyl; or two occurrences of R^{8B} are taken together with the nitrogen atom to which they are attached to form a 3-7 membered heterocyclic ring containing 1 or 2 heteroatoms independently selected from nitrogen and oxygen; or R^{8B} and R^{9B} are taken together with their intervening atoms to a form a 5-7 membered ring containing 1, 2, or 3 heteroatoms independently selected from nitrogen, oxygen, and sulfur;
R^{9B} is C₁₋₆ alkyl, C₁₋₆ haloalkyl, —(C₁₋₆ alkylene)-(C₃₋₆ cycloalkyl), or C₃₋₆ cycloalkyl; and
p and t are independently 0, 1, or 2.

In certain embodiments, the EPL is

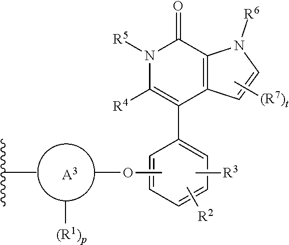

In certain embodiments, the EPL is

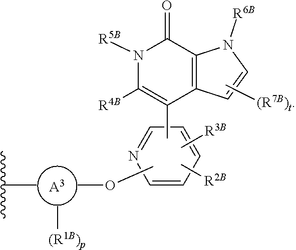

In certain embodiments, the EPL is

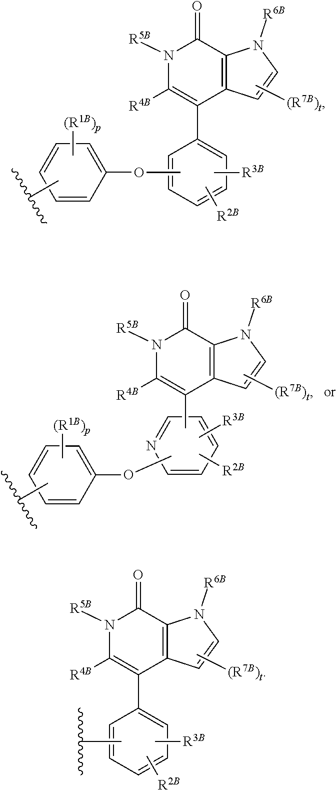

In certain embodiments, the EPL is

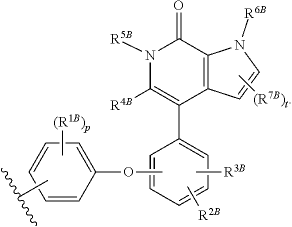

In certain embodiments, the EPL is

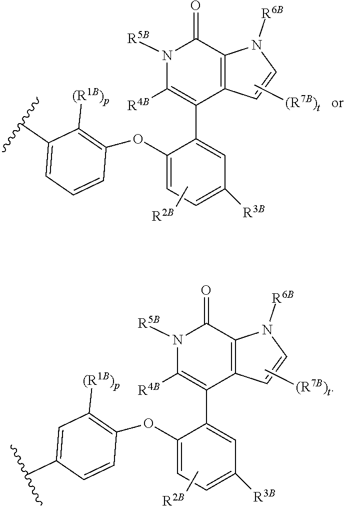

In certain embodiments, the EPL is

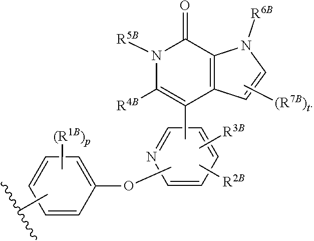

In certain embodiments, the EPL is

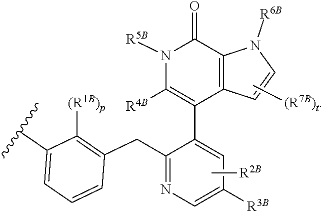

In certain embodiments, the EPL is

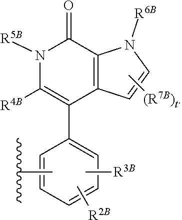

In certain embodiments the EPL is

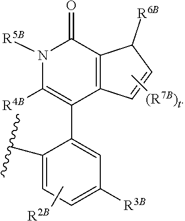

In certain embodiments, the EPL is

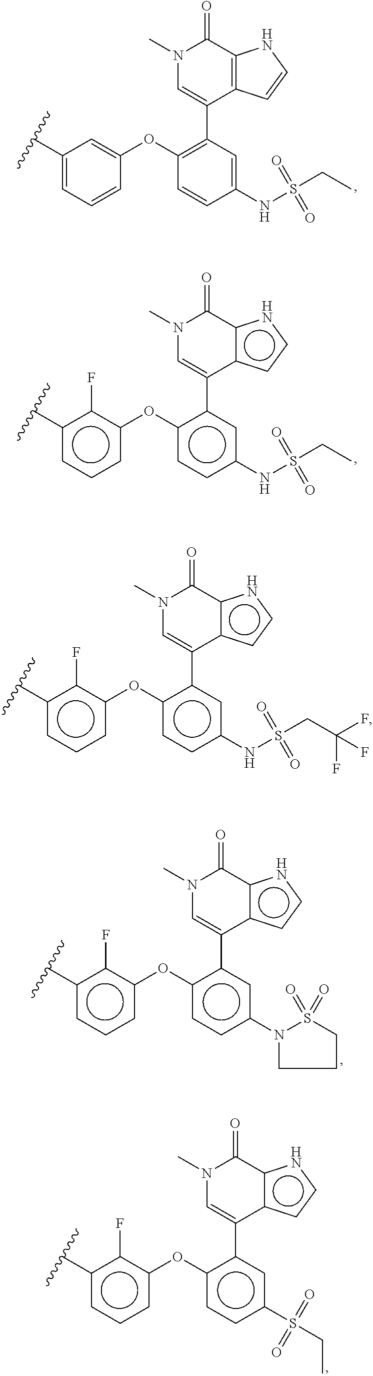

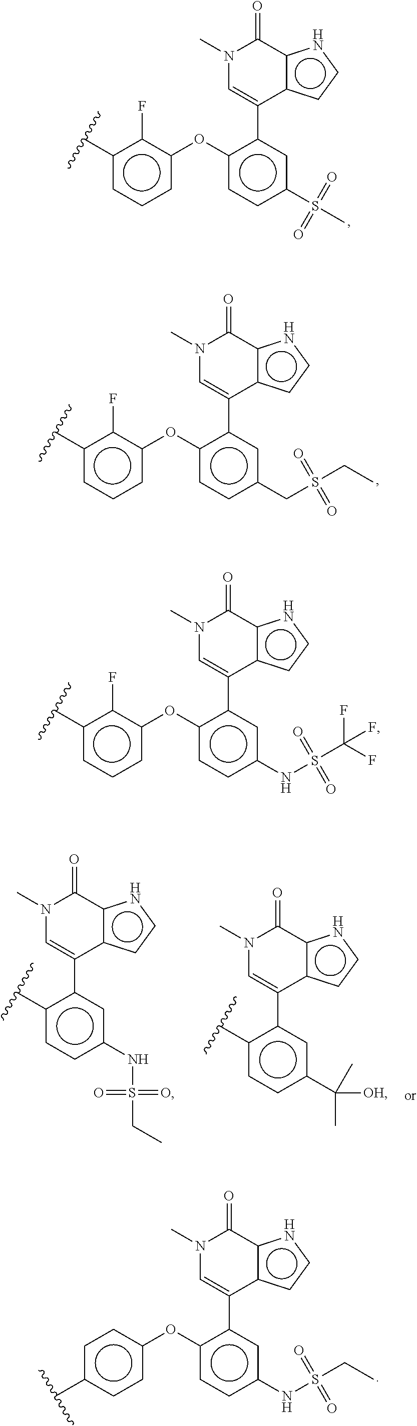

In certain embodiments, the EPL is

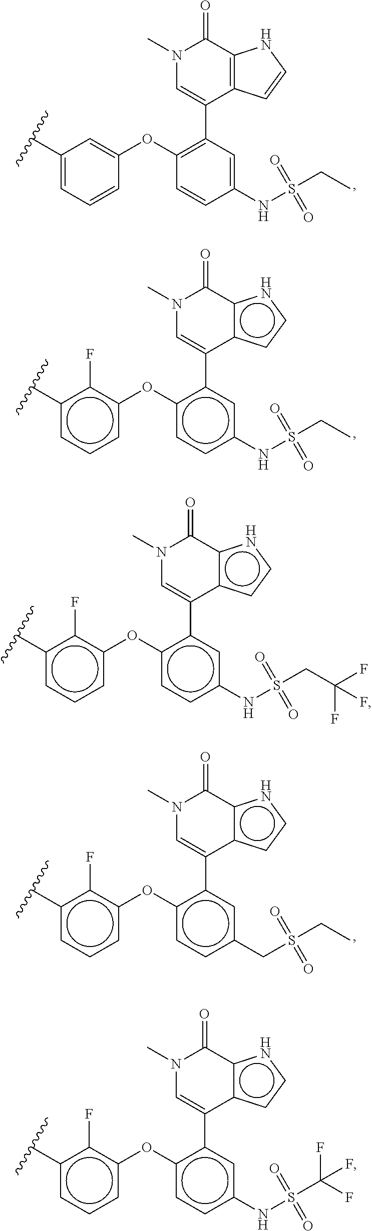

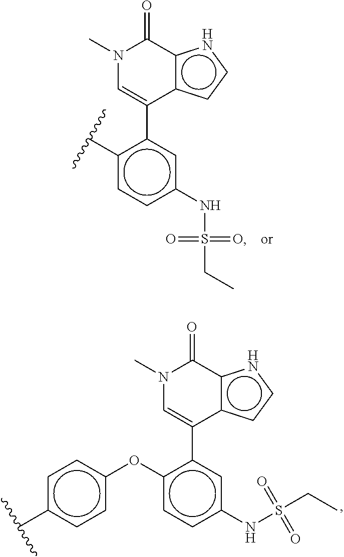

In certain embodiments, the EPL is

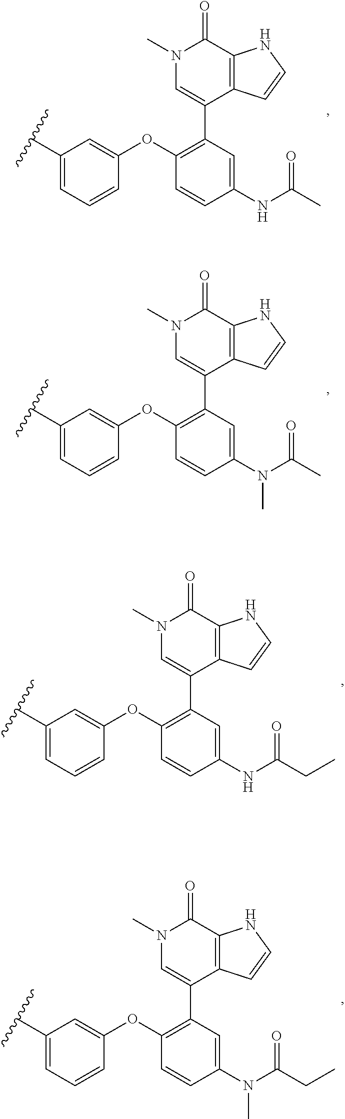 [SMILES: *c1cccc(Oc2ccc(NC(C)=O)cc2-c2cn(C)c(=O)c3[nH]ccc23)c1 ; *c1cccc(Oc2ccc(N(C)C(C)=O)cc2-c2cn(C)c(=O)c3[nH]ccc23)c1 ; *c1cccc(Oc2ccc(NC(=O)CC)cc2-c2cn(C)c(=O)c3[nH]ccc23)c1 ; *c1cccc(Oc2ccc(N(C)C(=O)CC)cc2-c2cn(C)c(=O)c3[nH]ccc23)c1]

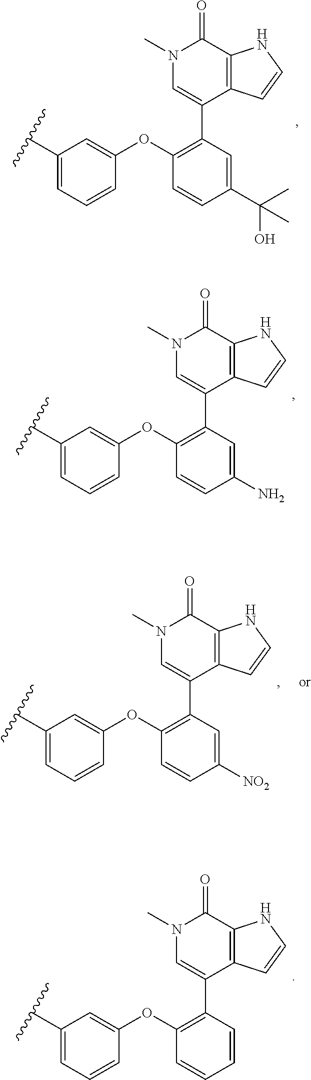 [SMILES: *c1cccc(Oc2ccc(C(C)(C)O)cc2-c2cn(C)c(=O)c3[nH]ccc23)c1 ; *c1cccc(Oc2ccc(N)cc2-c2cn(C)c(=O)c3[nH]ccc23)c1 ; *c1cccc(Oc2ccc([N+](=O)[O-])cc2-c2cn(C)c(=O)c3[nH]ccc23)c1 ; *c1cccc(Oc2ccccc2-c2cn(C)c(=O)c3[nH]ccc23)c1]

In certain embodiments, EPL is

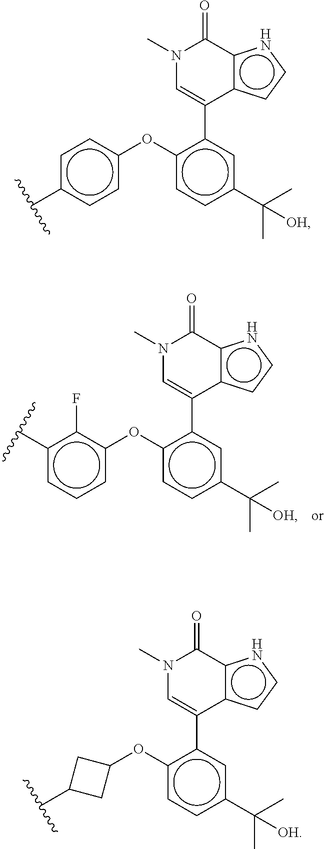

In certain embodiments, the EPL is

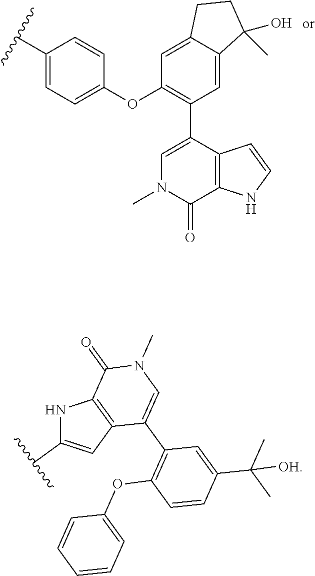 [SMILES: *c1ccc(Oc2cc3c(cc2-c2cn(C)c(=O)c4[nH]ccc24)C(C)(O)CC3)cc1 ; *c1cc2c(-c3cc(C(C)(C)O)ccc3Oc3ccccc3)cn(C)c(=O)c2[nH]1]

In certain embodiments, A³ is phenylene, pyridinylene, pyrazinylene, a 3-10 membered saturated monocyclic, bicyclic or spirocyclic carbocycylene, or C₁₋₆ alkylene. In certain embodiments, A³ is phenylene, a 3-10 membered saturated monocyclic, bicyclic or spirocyclic carbocycylene, or C₁₋₆ alkylene. In certain embodiments, A³ is phenylene. In certain embodiments, A³ is pyridinylene. In certain embodiments, A³ is pyrazinylene. In certain embodiments, A³ is a 3-10 membered saturated monocyclic, bicyclic or spirocyclic carbocycylene. In certain embodiments, A³ is C₁₋₆ alkylene. In certain embodiments, A³ is methylene or ethylene. In certain embodiments, A³ is a 3-6 membered saturated monocyclic carbocycylene. In certain embodiments, A³ is a 6-10 membered saturated bicyclic or spirocyclic carbocycylene. In certain embodiments, A³ is selected from the groups depicted in the compounds in Table 1 below.

As defined generally above, RB represents independently for each occurrence halo, C₁₋₄ alkyl, or C₁₋₄ haloalkyl. In certain embodiments, RB is halo. In certain embodiments, RB is fluoro. In certain embodiments, R^{1B} is C₁₋₄ alkyl. In certain embodiments, R^{1B} is C₁₋₄ haloalkyl. In certain embodiments, R^{1B} is selected from the groups depicted in the compounds in Table 1 below.

As defined generally above, R^{2B} is hydrogen, halo, C₁₋₄ alkyl, or C₁₋₄ haloalkyl. In certain embodiments, R²⁸ is hydrogen. In certain embodiments, R^{2B} is halo. In certain embodiments, R^{2B} is C₁₋₄ alkyl. In certain embodiments, R^{2B} is C₁₋₄ haloalkyl. In certain embodiments, R^{2B} is selected from the groups depicted in the compounds in Table 1 below.

As defined generally above, R^{3B} is —N(R^{8B})SO₂R^{9B}, —SO₂N(R^{8B})₂, —SO₂R^{9B}, —(C₁₋₆ alkylene)-SO₂R^{9B}, —(C₀₋₆ alkylene)-N(R^{8B})C(O)R^{9B}, —(C₀₋₆ alkylene)-C(O)N(R^{8B})₂, —N(R^{8B})₂, —NO₂, C₁₋₆ hydroxyalkyl, hydrogen, or a 4-7 membered saturated carbocyclic ring in which one CH₂ is replaced with SO₂. In certain embodiments, R^{3B} is —N(R^{8B})SO₂R^{9B}. In certain embodiments, R^{3B} is —SO₂R^{9B}. In certain embodiments, R^{3B} is —(C₁₋₆ alkylene)-SO₂R^{9B}. In certain embodiments, R^{3B} is —SO₂N(R^{8B})₂. In certain embodiments, R^{3B} is —(C₀₋₆ alkylene)-N(R^{8B})C(O)R^{9B}. In certain embodiments, R^{3B} is —N(R^{8B})C(O)R^{9B}. In certain embodiments, R^{3B} is —N(H)C(O)CH₃. In certain embodiments, R^{3B} is —N(CH₃)C(O)CH₃. In certain embodiments, R^{3B} is —(C₀₋₆ alkylene)-C(O)N(R^{8B})₂. In certain embodiments, R^{3B} is —C(O)N(R^{8B})₂. In certain embodiments, R^{3B} is —N(R^{8B})₂. In certain embodiments, R^{3B} is —NH₂. In certain embodiments, R^{3B} is —NO₂. In certain embodiments, R^{3B} is C₁₋₆ hydroxyalkyl. In certain embodiments, R^{3B} is —C(CH₃)₂(OH). In certain embodiments, R^{3B} is hydrogen. In certain embodiments, R^{3B} is a 4-7 membered saturated carbocyclic ring in which one CH₂ is replaced with SO₂. In certain embodiments, R^{3B} is selected from the groups depicted in the compounds in Table 1 below.

As defined generally above, R^{4B} is hydrogen, halo, or C₁₋₄ alkyl. In certain embodiments, R^{4B} is hydrogen. In certain embodiments, R^{4B} is halo. In certain embodiments, R^{4B} is C₁₋₄ alkyl. In certain embodiments, R^{4B} is selected from the groups depicted in the compounds in Table 1 below.

As defined generally above, R^{5B} is C₁₋₄ alkyl or C₃₋₄ cycloalkyl. In certain embodiments, R^{5B} is C₁₋₄ alkyl. In certain embodiments, R^{5B} is methyl. In certain embodiments, R^{5B} is C₃₋₄ cycloalkyl. In certain embodiments, R^{5B} is selected from the groups depicted in the compounds in Table 1 below.

As defined generally above, R^{6B} is hydrogen, C₁₋₄ alkyl, or C₃₋₄ cycloalkyl. In certain embodiments, R^{6B} is hydrogen. In certain embodiments, R^{3B} is C₁₋₄ alkyl. In certain embodiments, R^{6B} is C₃₋₄ cycloalkyl. In certain embodiments, R^{6B} is selected from the groups depicted in the compounds in Table 1 below.

As defined generally above, R^{7B} represents independently for each occurrence C₁₋₄ alkyl or C₃₋₄ cycloalkyl. In certain embodiments, R^{7B} is C₁₋₄ alkyl. In certain embodiments, R^{7B} is methyl. In certain embodiments, R^{7B} is C₃₋₄ cycloalkyl. In certain embodiments, R^{7B} is selected from the groups depicted in the compounds in Table 1 below.

As defined generally above, R^{8B} represents independently for each occurrence hydrogen or C₁₋₄ alkyl; or two occurrences of R^{8B} are taken together with the nitrogen atom to which they are attached to form a 3-7 membered heterocyclic ring containing 1 or 2 heteroatoms independently selected from nitrogen and oxygen; or R^{8B} and R^{9B} are taken together with their intervening atoms to a form a 5-7 membered ring containing 1, 2, or 3 heteroatoms independently selected from nitrogen, oxygen, and sulfur. In certain embodiments, R^{8B} represents independently for each occurrence hydrogen or C₁₋₄ alkyl. In certain embodiments, R^{8B} is hydrogen. In certain embodiments, R^{8B} is C₁₋₄ alkyl. In certain embodiments, two occurrences of R^{8B} are taken together with the nitrogen atom to which they are attached to form a 3-7 membered heterocyclic ring containing 1 or 2 heteroatoms independently selected from nitrogen and oxygen. In certain embodiments, R^{8B} and R^{9B} are taken together with their intervening atoms to a form a 5-7 membered ring containing 1, 2, or 3 heteroatoms independently selected from nitrogen, oxygen, and sulfur. In certain embodiments, R^{8B} is selected from the groups depicted in the compounds in Table 1 below.

As defined generally above, R^{9B} is C₁₋₆ alkyl, C₁₋₆ haloalkyl, —(C₁₋₆ alkylene)-(C₃₋₆ cycloalkyl), or C₃₋₆ cycloalkyl. In certain embodiments, R^{9B} is C₁₋₆ alkyl or C₁₋₆ haloalkyl. In certain embodiments, R^{9B} is C₁₋₆ alkyl. In certain embodiments, R^{9B} is C₁₋₆ haloalkyl. In certain embodiments, R^{9B} is —(C₁₋₆ alkylene)-(C₃₋₆ cycloalkyl). In certain embodiments, R^{9B} is C₃₋₆ cycloalkyl. In certain embodiments, R^{9B} is selected from the groups depicted in the compounds in Table 1 below.

As defined generally above, k is 1, 2, 3, or 4. In certain embodiments, k is 1. In certain embodiments, k is 2. In certain embodiments, k is 3. In certain embodiments, k is 4. In certain embodiments, k is selected from the corresponding value in the groups depicted in the compounds in Table 1 below.

In certain embodiments, p is 2. In certain embodiments, p is 1. In certain embodiments, p is 0. In certain embodiments, t is 2. In certain embodiments, t is 0. In certain embodiments, t is 1. In certain embodiments, p is selected from the corresponding value in the groups depicted in the compounds in Table 1 below. In certain embodiments, t is selected from the corresponding value in the groups depicted in the compounds in Table 1 below.

In certain embodiments, the EPL is selected from the groups depicted in the compounds in Table 1 below.

In certain embodiments, the EPL is defined by variable A² set forth above in connection with Formula I. In certain embodiments, the EPL is defined by one or more of the embodiments for variable A² set forth in connection with Formula I.

The compounds may be further characterized according to, for example, the identity of L. Exemplary further embodiments for L are provided in Part F below.

Another aspect of the invention provides a compound represented by Formula II-A:

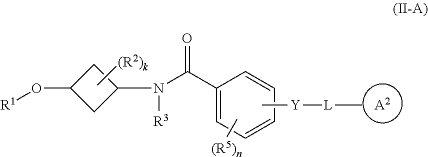

or a pharmaceutically acceptable salt thereof, wherein:
R¹ is phenyl substituted by cyano, halogen, and m occurrences of R⁴;
R² represents independently for each occurrence C₁₋₄ alkyl;
R³ is hydrogen or C₁₋₄ alkyl;
R⁴ is C₁₋₄ alkyl;
R⁵ represents independently for each occurrence C₁₋₄ alkyl or halogen;
A²

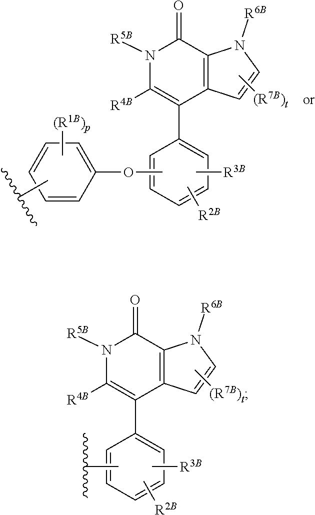

R^{1B} represents independently for each occurrence halo, C₁₋₄ alkyl, or C₁₋₄ haloalkyl;
R^{2B} is hydrogen, halo, C₁₋₄ alkyl, or C₁₋₄ haloalkyl;
R^{3B} is —N(R^{8B})SO₂R^{9B}, —SO₂N(R^{8B})₂, —SO₂R^{9B}, —(C₁₋₆ alkylene)-SO₂R^{9B}, —(C₀₋₆ alkylene)-N(R^{8B})C(O)R^{9B}, —(C₀₋₆ alkylene)-C(O)N(R^{8B})₂, —N(R^{8B})₂, —NO₂, C₁₋₆ hydroxyalkyl, or hydrogen;
R^{4B} is hydrogen, halo, or C₁₋₄ alkyl;
R^{5B} is C₁₋₄ alkyl or C₃₋₄ cycloalkyl;
R^{6B} is hydrogen, C₁₋₄ alkyl, or C₃₋₄ cycloalkyl;
R^{7B} represents independently for each occurrence C₁₋₄ alkyl or C₃₋₄ cycloalkyl;
R^{8B} represents independently for each occurrence hydrogen or C₁₋₄ alkyl;
R^{9B} is C₁₋₆ alkyl, C₁₋₆ haloalkyl, —(C₁₋₆ alkylene)-(C₃₋₆ cycloalkyl), or C₃₋₆ cycloalkyl; and
L is a linker; and
k is 1, 2, 3, or 4; and
m, n, p and t are independently 0, 1, or 2.

The definitions of variables in Formula II-A above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula II-A.

As define generally above, R¹ is phenyl substituted by cyano, halogen, and m occurrences of R⁴. In certain embodiments, R¹ is phenyl substituted by cyano, halogen, and 0 occurrences of R⁴. In certain embodiments, R¹ is phenyl substituted by cyano, halogen, and 1 occurrence of R⁴. In certain embodiments, R¹ is phenyl substituted by cyano, halogen, and 2 occurrences of R⁴. In certain embodiments, R¹ is phenyl substituted by cyano, Cl, and 0 occurrences of R⁴. In certain embodiments, R¹ is

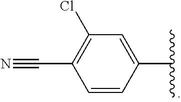 [SMILES: *c1ccc(C#N)c(Cl)c1]

In certain embodiments, R¹ is selected from the groups depicted in the compounds in Table 1 below.

As defined generally above, R² represents independently for each occurrence C₁₋₄ alkyl. In certain embodiments, R² is C₁₋₂ alkyl. In certain embodiments, R² is methyl. In certain embodiments, R² is selected from the groups depicted in the compounds in Table 1 below.

As defined generally above, R³ is hydrogen or C₁₋₄ alkyl. In certain embodiments, R³ is hydrogen. In certain embodiments, R³ is C₁₋₄ alkyl. In certain embodiments, R³ is selected from the groups depicted in the compounds in Table 1 below.

In certain embodiments, Y is —N(C₁₋₄ alkyl)-. In certain embodiments, Y is —N(CH₃)—. In certain embodiments, Y is a 3-7 membered saturated heterocyclylene containing 1 or 2 heteroatoms selected from oxygen and nitrogen. In certain embodiments, Y is piperidinylene.

As defined generally above, A²

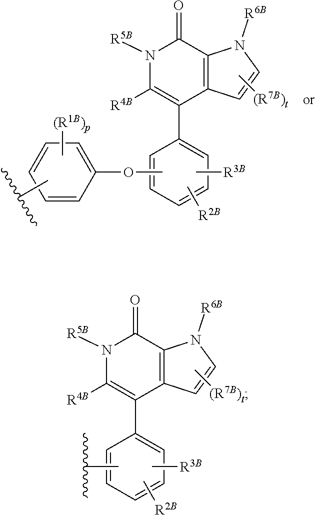

In certain embodiments, A²

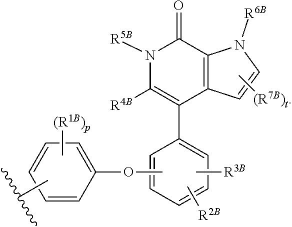

embodiments, A² is

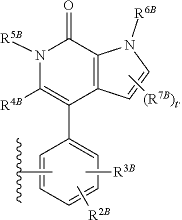

In certain embodiments, the compound of Formula II-A is further defined by Formula IIa-A or a pharmaceutically acceptable salt thereof:

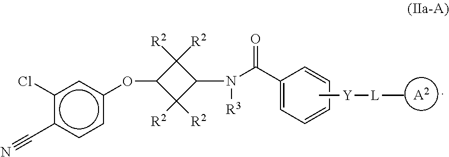

In certain embodiments, the definition of variables R², R³, A¹, and A² is one of the embodiments described above in connection with Formula II-A.

In certain embodiments, the compound of Formula II-A is further defined by Formula IIb-A or a pharmaceutically acceptable salt thereof.

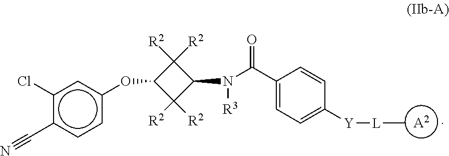

In certain embodiments, the definition of variables R², R³, A¹, and A² is one of the embodiments described above in connection with Formula II-A.

As defined generally above, R^{1B} represents independently for each occurrence halo, C₁₋₄ alkyl, or C₁₋₄ haloalkyl. In certain embodiments, RB is halo. In certain embodiments, RB is fluoro. In certain embodiments, RB is C₁₋₄ alkyl. In certain embodiments, R^{1B} is C₁₋₄ haloalkyl. In certain embodiments, R^{1B} is selected from the groups depicted in the compounds in Table 1 below.

As defined generally above, R^{2B} is hydrogen, halo, C₁₋₄ alkyl, or C₁₋₄ haloalkyl. In certain embodiments, R^{2B} is hydrogen. In certain embodiments, R^{2B} is halo. In certain embodiments, R^{2B} is C₁₋₄ alkyl. In certain embodiments, R^{2B} is C₁₋₄ haloalkyl. In certain embodiments, R^{2B} is selected from the groups depicted in the compounds in Table 1 below.

As defined generally above, R^{3B} is —N(R^{B})SO₂R^{9B}, —SO₂N(R^{8B})₂, —SO₂R⁹⁸, —(C₁₋₆ alkylene)-SO₂R^{9B}, —(C₀₋₆ alkylene)-N(R^{8B})C(O)R^{9B}, —(C₀₋₆ alkylene)-C(O)N(R^{SB})₂, —N(R^{8B})₂, —NO₂, C₁₋₆ hydroxyalkyl, or hydrogen. In certain embodiments, R^{3B} is —N(R^{8B})SO₂R^{9B}. In certain embodiments, R^{3B} is —SO₂R^{9B}. In certain embodiments, R^{3B} is —(C₁₋₆ alkylene)-SO₂R^{9B}. In certain embodiments, R^{3B} is —SO₂N(R^{8B})₂. In certain embodiments, R^{3B} is —(C₀₋₅ alkylene)-N(R^{8B})C(O)R^{9B}. In certain embodiments, R³ is —N(R^{8B})C(O)R^{9B}. In certain embodiments, R^{3B} is —N(H)C(O)CH₃. In certain embodiments, R^{3B} is —N(CH₃)C(O)CH₃. In certain embodiments, R^{3B} is —(C_{0.6} alkylene)-C(O)N(R^{3B})₂. In certain embodiments, R^{3E} is —C(O)N(R^{B})₂. In certain embodiments, R^{3B} is —N(R^{8B})₂. In certain embodiments, R^{3B} is —NH₂. In certain embodiments, R^{3B} is —NO₂. In certain embodiments, R^{3B} is C₁₋₆ hydroxyalkyl. In certain embodiments, R^{3B} is —C(CH₃)₂(OH). In certain embodiments, R^{3B} is hydrogen. In certain embodiments, R^{3B} is selected from the groups depicted in the compounds in Table 1 below.

As defined generally above, R^{4B} is hydrogen, halo, or C₁₋₄ alkyl. In certain embodiments, R^{4B} is hydrogen. In certain embodiments, R^{4B} is halo. In certain embodiments, R^{4B} is C₁₋₄ alkyl. In certain embodiments, R^{4B} is selected from the groups depicted in the compounds in Table 1 below.

As defined generally above, R^{5B} is C₁₋₄ alkyl or C₃₋₄ cycloalkyl. In certain embodiments, R^{5B} is C₁₋₄ alkyl. In certain embodiments, R^{8B} is methyl. In certain embodiments, R^{5B} is C₃₋₄ cycloalkyl. In certain embodiments, R^{3B} is selected from the groups depicted in the compounds in Table 1 below.

As defined generally above, R^{6B} is hydrogen, C₁₋₄ alkyl, or C₃₋₄ cycloalkyl. In certain embodiments, R^{6B} is hydrogen. In certain embodiments, R^{6B} is C₁₋₄ alkyl. In certain embodiments, R^{6B} is C₃₋₄ cycloalkyl. In certain embodiments, R^{6B} is selected from the groups depicted in the compounds in Table 1 below.

As defined generally above, R^{7B} represents independently for each occurrence C₁₋₄ alkyl or C₃₋₄ cycloalkyl. In certain embodiments, R^{7B} is C₁₋₄ alkyl. In certain embodiments, R^{7B} is methyl. In certain embodiments, R^{7B} is C₃₋₄ cycloalkyl. In certain embodiments, R^{7B} is selected from the groups depicted in the compounds in Table 1 below.

As defined generally above, R^{8B} represents independently for each occurrence hydrogen or C₁₋₄ alkyl. In certain embodiments, R^{8B} is hydrogen. In certain embodiments, R^{8B} is C₁₋₄ alkyl. In certain embodiments, R^{8B} is selected from the groups depicted in the compounds in Table 1 below.

As defined generally above, R^{9B} is C₁₋₆ alkyl, C₁₋₆ haloalkyl, —(C₁₋₆ alkylene)-(C₃₋₆ cycloalkyl), or C₃₋₆ cycloalkyl. In certain embodiments, R^{9B} is C₁₋₆ alkyl or C₁₋₆ haloalkyl. In certain embodiments, R^{9B} is C₁₋₆ alkyl. In certain embodiments, R^{9B} is C₁₋₆ haloalkyl. In certain embodiments, R^{9B} is —(C₁₋₆ alkylene)-(C₃₋₆ cycloalkyl). In certain embodiments, R^{9B} is C₃₋₆ cycloalkyl. In certain embodiments, R^{9B} is selected from the groups depicted in the compounds in Table 1 below.

As defined generally above, k is 1, 2, 3, or 4. In certain embodiments, k is 1. In certain embodiments, k is 2. In certain embodiments, k is 3. In certain embodiments, k is 4. In certain embodiments, k is selected from the corresponding value in the groups depicted in the compounds in Table 1 below.

As defined generally above, m, n, p and t are independently 0, 1, or 2. In certain embodiments, p is 2. In certain embodiments, p is 1. In certain embodiments, p is 0. In certain embodiments, t is 2. In certain embodiments, m is 0. In certain embodiments, m is 1. In certain embodiments, m is 2. In certain embodiments, n is 0. In certain embodiments, n is 1. In certain embodiments, n is 2. In certain embodiments, t is 0. In certain embodiments, t is 1. In certain embodiments, p is selected from the corresponding value in the groups depicted in the compounds in Table 1 below. In certain embodiments, t is selected from the corresponding value in the groups depicted in the compounds in Table 1 below. In certain embodiments, m is selected from the corresponding value in the groups depicted in the compounds in Table 1 below. In certain embodiments, n is selected from the corresponding value in the groups depicted in the compounds in Table 1 below.

In certain embodiments, A² is

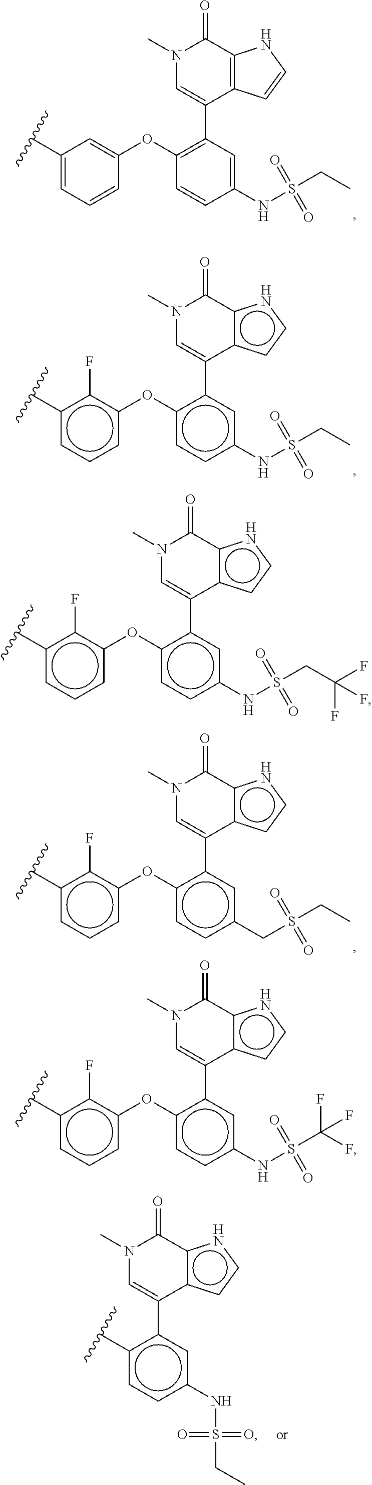

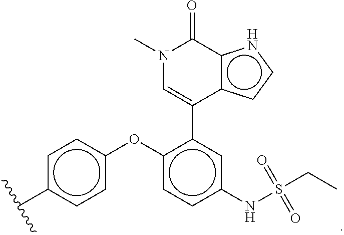

In certain embodiments, A² is

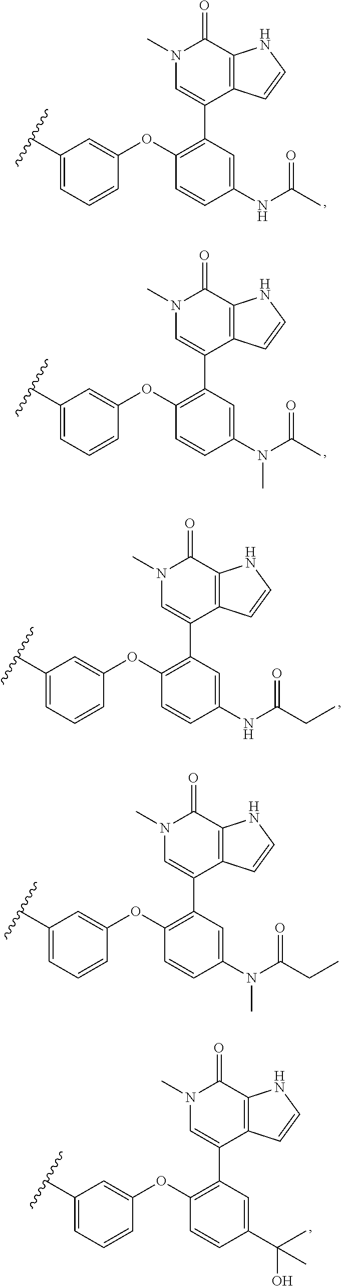 [SMILES: *c1cccc(Oc2ccc(NC(C)=O)cc2-c2cn(C)c(=O)c3[nH]ccc23)c1 ; *c1cccc(Oc2ccc(N(C)C(C)=O)cc2-c2cn(C)c(=O)c3[nH]ccc23)c1 ; *c1cccc(Oc2ccc(NC(=O)CC)cc2-c2cn(C)c(=O)c3[nH]ccc23)c1 ; *c1cccc(Oc2ccc(N(C)C(=O)CC)cc2-c2cn(C)c(=O)c3[nH]ccc23)c1 ; *c1cccc(Oc2ccc(C(C)(C)O)cc2-c2cn(C)c(=O)c3[nH]ccc23)c1]

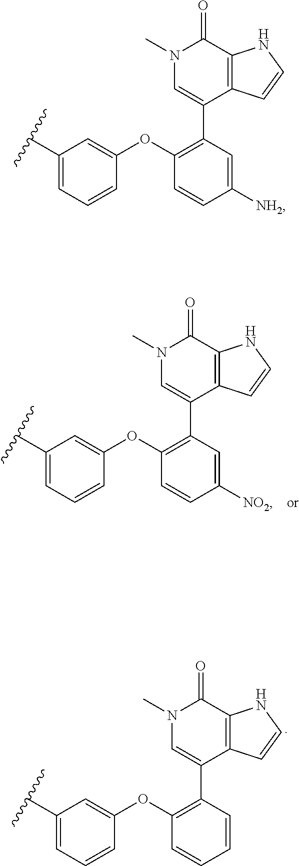 [SMILES: *c1cccc(Oc2ccc(N)cc2-c2cn(C)c(=O)c3[nH]ccc23)c1 ; *c1cccc(Oc2ccc([N+](=O)[O-])cc2-c2cn(C)c(=O)c3[nH]ccc23)c1 ; *c1cccc(Oc2ccccc2-c2cn(C)c(=O)c3[nH]ccc23)c1]

The compounds may be further characterized according to, for example, the identity of L. Exemplary further embodiments for L are provided in Part F below.

### Part E: Compound of Formula III

Another aspect of the invention provides a compound represented by Formula III:

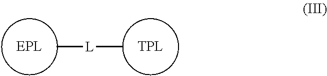

or a pharmaceutically acceptable salt thereof; wherein:
TPL is a group defined by Formula III-I that is substituted by one occurrence of R^{III-1A}, wherein Formula III-1 is represented by:

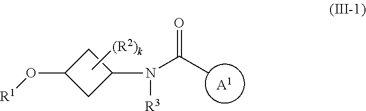

or a pharmaceutically acceptable salt thereof; wherein:
R^{II-1A} is a bond to L;
R¹ is phenyl substituted by cyano, halogen, and m occurrences of R⁴;
R² represents independently for each occurrence C₁₋₄ alkyl;
R³ is hydrogen or C₁₋₄ alkyl;
R⁴ is C₁₋₄ alkyl;
R⁵ represents independently for each occurrence C₁₋₄ alkyl or halogen, or one occurrence of R⁵ is taken together with R³ to form a C₁₋₃ alkylene;
A¹ is a pyridazinyl, pyrimidinyl, pyrazinyl, or pyridinyl, each of which is substituted with n occurrences of R⁵;
L is a linker;
EPL is a moiety that binds to BRD4;
k is 1, 2, 3, or 4; and
m and n are independently 0, 1, or 2.

The definitions of variables in Formula III-1 above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula III-1.

As defined generally above, R⁵ represents independently for each occurrence C₁₋₄ alkyl or halogen, or one occurrence of R⁵ is taken together with R³ to form a C₁₋₃ alkylene. In certain embodiments, R⁵ represents independently for each occurrence C₁₋₄ alkyl or halogen. In certain embodiments, R⁵ represents independently for each occurrence C₁₋₄ alkyl. In certain embodiments, R⁵ represents independently for each occurrence methyl. In certain embodiments, R⁵ represents independently for each occurrence halogen. In certain embodiments, R⁵ represents independently for each occurrence Cl, Br, or F.

In certain embodiments, R⁵ is C₁₋₄ alkyl. In certain embodiments, R⁵ is methyl. In certain embodiments, R⁵ is halogen. In certain embodiments, R⁵ is Cl, Br, or F. In certain embodiments, one occurrence of R⁵ is taken together with R³ to form a C₁₋₃ alkylene. In certain embodiments, R⁵ is selected from the groups depicted in the compounds in Table 1 below.

In certain embodiments, the definition of one or more of TPL, R¹, R², R³, R⁴, R⁵, A¹, L, EPL, k m, and n are as set forth above for Formula II or an embodiment in connection with Formula II.

### Part F: Exemplary Further Description of Linker (L) Component of Compounds of Formula I and II

Compounds of Formulae I, I-1, II and II-1 may be further characterized according to, for example, the identity of the linker (L) component. A variety of linkers are known to one of skill in the art and may be used in the heterobifunctional compounds described herein. For example, in certain embodiments, L comprises one or more optionally substituted groups selected from amino acids, polyether chains, aliphatic groups, and any combinations thereof. In certain embodiments, L consists of one or more optionally substituted groups selected from amino acids, polyether chains, aliphatic groups, and any combinations thereof. In certain embodiments, L consists of one or more groups selected from amino acids, polyether chains, aliphatic groups, and any combinations thereof.

In some embodiments, L is symmetrical. In some embodiments, L is asymmetric. In certain embodiments, L is a bond.

In certain embodiments, L is a covalent bond or a bivalent C₁₋₃₀ saturated or unsaturated, straight or branched, hydrocarbon chain, wherein 1-15 methylene units of L are optionally and independently replaced by cyclopropylene, —N(H)—, —N(C₁₋₄ alkyl)-, —N(C₃₋₅ cycloalkyl)-, —O—, —C(O)—, —OC(O)—, —C(O)O—, —C(O)—, —S—, —S(O)—, —S(O)₂—, —S(O)₂N(H)—, —S(O)₂N(C₁₋₄ alkyl)-, —S(O)₂N(C₃₋₅ cycloalkyl)-, —N(H)C(O)—, —N(C₁₋₄ alkyl)C(O)—, —N(C₃₋₅ cycloalkyl)C(O)—, —C(O)N(H)—, —C(O)N(C₁₋₄ alkyl)-, —C(O)N(C₃₋₅ cycloalkyl)-, —N(C₃₋₆ cycloalkyl)-, —C(H)(C₃₋₆ cycloalkyl)-, phenylene, an 8-10 membered bicyclic arylene, a 4-7 membered saturated or partially unsaturated carbocyclylene, an 8-10 membered bicyclic saturated or partially unsaturated carbocyclylene, a 3-7 membered saturated or partially unsaturated heterocyclylene having 1-2 heteroatoms independently selected from nitrogen, oxygen, and sulfur, an 8-11 membered bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur.

In certain embodiments, L is a covalent bond or a bivalent C₁₋₃₀ saturated or unsaturated, straight or branched, hydrocarbon chain, wherein 1-15 methylene units of L are optionally and independently replaced by cyclopropylene, —N(H)—, —N(C₁₋₄ alkyl)-, —N(C₃₋₅ cycloalkyl)-, —O—, —C(O)—, —OC(O)—, —C(O)O—, —S—, —S(O)—, —S(O)₂—, —S(O)₂N(H)—, —S(O)₂N(C₁₋₄ alkyl)-, —S(O)₂N(C₃₋₅ cycloalkyl)-, —N(H)C(O)—, —N(C₁₋₄ alkyl)C(O)—, —N(C₃₋₅ cycloalkyl)C(O)—, —C(O)N(H)—, —C(O)N(C₁₋₄ alkyl)-, —C(O)N(C₃₋₅ cycloalkyl)-, phenylene, an 8-10 membered bicyclic arylene, a 4-7 membered saturated or partially unsaturated carbocyclylene, an 8-10 membered bicyclic saturated or partially unsaturated carbocyclylene, a 3-7 membered saturated or partially unsaturated heterocyclylene having 1-2 heteroatoms independently selected from nitrogen, oxygen, and sulfur, an 8-10 membered bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur.

In certain embodiments, L is a bivalent, saturated or unsaturated, straight or branched C₁₋₆₀ hydrocarbon chain, wherein 0-20 methylene units of the hydrocarbon are independently replaced with —O—, —S—, —N(R**)—, —OC(O)—, —C(O)O—, —C(O)—, —S(O)—, —S(O)₂—, —N(R**)S(O)₂—, —S(O)₂N(R**)—, —N(R**)C(O)—, —C(O)N(R**)—, —OC(O)N(R**)—, —N(R**)C(O)O—, —N(C₃₋₆ cycloalkyl)-, —C(H)(C₃₋₆ cycloalkyl)-, optionally substituted 3-10 membered carbocyclyl, or optionally substituted 3-11 membered heterocyclyl containing 1, 2, 3, or 4 heteroatoms independently selected from nitrogen, oxygen, and sulfur, wherein R** represents independently for each occurrence hydrogen, C₁₋₆ alkyl, or C₃₋₆ cycloalkyl.

In certain embodiments, L is a bivalent, saturated or unsaturated, straight or branched C₁₋₆₀ hydrocarbon chain, wherein 0-20 methylene units of the hydrocarbon are independently replaced with —O—, —S—, —N(R**)—, —OC(O)—, —C(O)O—, —S(O)—, —S(O)₂—, —N(R**)S(O)₂—, —S(O)₂N(R**)—, —N(R**)C(O)—, —C(O)N(R**)—, —OC(O)N(R**)—, —N(R**)C(O)O—, optionally substituted 3-10 membered carbocyclyl, or optionally substituted 3-10 membered heterocyclyl containing 1, 2, 3, or 4 heteroatoms independently selected from nitrogen, oxygen, and sulfur, wherein R** represents independently for each occurrence hydrogen, C₁₋₆ alkyl, or C₃₋₆ cycloalkyl.

In certain embodiments, L is a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, or optionally substituted 3-10 membered heterocyclyl containing 1, 2, 3, or 4 heteroatoms independently selected from nitrogen, oxygen, and sulfur.

In certain embodiments, L is a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —C(O)—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, —N(C₃₋₆ cycloalkyl)-, —C(H)(C₃₋₆ cycloalkyl)-, optionally substituted 3-10 membered carbocyclyl, or optionally substituted 3-11 membered heterocyclyl containing 1, 2, 3, or 4 heteroatoms independently selected from nitrogen, oxygen, and sulfur.

In certain embodiments, L is a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, or optionally substituted 3-10 membered heterocyclyl containing 1, 2, 3, or 4 heteroatoms independently selected from nitrogen, oxygen, and sulfur.

In certain embodiments, L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —N(H)C(O)—, —N(C₁₋₆ alkyl)C(O)—, —C(O)N(H)—, —C(O)N(C₁₋₆ alkyl)-, 3-10 membered carbocyclyl, or 3-10 membered heterocyclyl containing 1, 2, or 3 heteroatoms independently selected from nitrogen, oxygen, and sulfur.

In certain embodiments, L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —C(O)—, —N(H)C(O)—, —N(C₁₋₆ alkyl)C(O)—, —C(O)N(H)—, —C(O)N(C₁₋₆ alkyl)-, —N(C₃₋₆ cycloalkyl)-, —C(H)(C₃₋₆ cycloalkyl)-, 3-10 membered carbocyclyl, or 3-11 membered heterocyclyl containing 1, 2, or 3 heteroatoms independently selected from nitrogen, oxygen, and sulfur.

In certain embodiments, L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —N(H)C(O)—, —N(C₁₋₆ alkyl)C(O)—, —C(O)N(H)—, —C(O)N(C₁₋₆ alkyl)-, 3-10 membered carbocyclyl, or 3-10 membered heterocyclyl containing 1, 2, or 3 heteroatoms independently selected from nitrogen, oxygen, and sulfur.

In certain embodiments, L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —N(H)C(O)—, —N(C₁₋₆ alkyl)C(O)—, —C(O)N(H)—, or —C(O)N(C₁₋₆ alkyl)-.

In certain embodiments, L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —C(O)—, —N(H)C(O)—, —N(C₁₋₆ alkyl)C(O)—, —C(O)N(H)—, —C(O)N(C₁₋₆ alkyl)-, —N(C₃₋₆ cycloalkyl)-, or —C(H)(C₃₋₆ cycloalkyl)-.

In certain embodiments, L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —N(H)C(O)—, —N(C₁₋₆ alkyl)C(O)—, —C(O)N(H)—, or —C(O)N(C₁₋₆ alkyl)-.

In yet other embodiments, L comprises a polyethylene glycol chain ranging in size from about 1 to about 12 ethylene glycol units, from about 1 to about 10 ethylene glycol units, from about 2 to about 6 ethylene glycol units, from about 2 to about 5 ethylene glycol units, or from about 2 to about 4 ethylene glycol units. In yet other embodiments, L is a diradical of a polyethylene glycol chain ranging in size from about 1 to about 12 ethylene glycol units, from about 1 to about 10 ethylene glycol units, from about 2 to about 6 ethylene glycol units, from about 2 to about 5 ethylene glycol units, or from about 2 to about 4 ethylene glycol units.

In certain embodiments, L is a heteroalkylene having from 4 to 30 atoms selected from carbon, oxygen, nitrogen, and sulfur. In certain embodiments, L is a heteroalkylene having from 4 to 20 atoms selected from carbon, oxygen, nitrogen, and sulfur. In certain embodiments, L is a heteroalkylene having from 4 to 10 atoms selected from carbon, oxygen, nitrogen, and sulfur. In certain embodiments, L is a heteroalkylene having from 4 to 30 atoms selected from carbon, oxygen, and nitrogen. In certain embodiments, L is a heteroalkylene having from 4 to 20 atoms selected from carbon, oxygen, and nitrogen. In certain embodiments, L is a heteroalkylene having from 4 to 10 atoms selected from carbon, oxygen, and nitrogen. In certain embodiments, L is a heteroalkylene having from 4 to 30 atoms selected from carbon and oxygen. In certain embodiments, L is a heteroalkylene having from 4 to 20 atoms selected from carbon and oxygen. In certain embodiments, L is a heteroalkylene having from 4 to 10 atoms selected from carbon and oxygen.

In additional embodiments, the L is an optionally substituted (poly)ethyleneglycol having between 1 and about 100 ethylene glycol units, between about 1 and about 50 ethylene glycol units, between 1 and about 25 ethylene glycol units, between about 1 and about 10 ethylene glycol units, between 1 and about 8 ethylene glycol units, between 1 and about 6 ethylene glycol units, between 2 and about 4 ethylene glycol units, or optionally substituted alkyl groups interdispersed with optionally substituted, O, N, S, P or Si atoms. In certain embodiments, L is substituted with an aryl, phenyl, benzyl, alkyl, alkylene, or heterocycle group.

In certain embodiments, L is a bivalent, saturated or unsaturated, straight or branched C₁₋₄₅ hydrocarbon chain, wherein 0-10 methylene units of the hydrocarbon are independently replaced with —O—, —S—, —N(R**)—, —OC(O)—, —C(O)O—, —S(O)—, —S(O)₂—, —N(R**)S(O)₂—, —S(O)₂N(R**)—, —N(R**)C(O)—, —C(O)N(R**)—, —OC(O)N(R**)—, —N(R**)C(O)O—, optionally substituted carbocyclyl, or optionally substituted heterocyclyl, wherein R** represents independently for each occurrence hydrogen, C₁₋₆ alkyl, or C₃₋₆ cycloalkyl.

In certain embodiments, L is a bivalent, saturated or unsaturated, straight or branched C₁₋₄₅ hydrocarbon chain, wherein 0-10 methylene units of the hydrocarbon are independently replaced with —O—, —S—, —N(R**)—, —OC(O)—, —C(O)O—, —S(O)—, —S(O)₂—, —N(R**)S(O)₂—, —S(O)₂N(R**)—, —N(R**)C(O)—, —C(O)N(R**)—, —OC(O)N(R**)—, —N(R**)C(O)O—, optionally substituted 3-10 membered carbocyclyl, or optionally substituted 3-10 membered heterocyclyl containing 1, 2, 3, or 4 heteroatoms selected from nitrogen, oxygen, and sulfur, wherein R** represents independently for each occurrence hydrogen, C₁₋₆ alkyl, or C₃₋₆ cycloalkyl.

In certain embodiments, L has the formula —N(R)-(optionally substituted 3-20 membered heteroalkylene)ₚ-CH₂—C(O)—, wherein R is hydrogen or optionally substituted C₁-C₆ alkyl, and p is 0 or 1.

In certain embodiments, L has the formula —N(R)-(3-20 membered heteroalkylene)ₚ-CH₂—C(O)—; wherein the 3-20 membered heteroalkylene is optionally substituted with 1, 2, 3, or 4 substituents independently selected from halogen, C₁-C₆ haloalkyl, C₃-C₆ cycloalkyl, hydroxyl, and cyano; R is hydrogen or optionally substituted C₁-C₆ alkyl; and p is 0 or 1.

In certain embodiments, L has the formula —N(R)-(3-20 membered heteroalkylene)ₚ-CH₂—C(O)—; wherein the 3-20 membered heteroalkylene is optionally substituted with 1, 2, or 3 substituents independently selected from halogen and C₁-C₆ haloalkyl; R is hydrogen or C₁-C₆ alkyl; and p is 0 or 1.

In certain embodiments, L is a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, or optionally substituted 3-10 membered heterocyclyl containing 1, 2, 3, or 4 heteroatoms independently selected from nitrogen, oxygen, and sulfur.

In certain embodiments, L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —N(H)C(O)—, —N(C₁₋₆ alkyl)C(O)—, —C(O)N(H)—, —C(O)N(C₁₋₆ alkyl)-, 3-10 membered carbocyclyl, or 3-10 membered heterocyclyl containing 1, 2, or 3 heteroatoms independently selected from nitrogen, oxygen, and sulfur.

In certain embodiments, L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —N(H)C(O)—, —N(C₁₋₆ alkyl)C(O)—, —C(O)N(H)—, or —C(O)N(C₁₋₆ alkyl)-.

In certain embodiments, L is a bivalent, saturated or unsaturated, straight or branched C₅₋₄₀ hydrocarbon chain, wherein 1-20 methylene units of the hydrocarbon are independently replaced with —O—, —N(H)—, —N(C₁₋₆ alkyl)-, —N(H)C(O)—, —N(C₁₋₆ alkyl)C(O)—, —C(O)N(H)—, —C(O)N(C₁₋₆ alkyl)-, optionally substituted 3-10 membered carbocyclyl, or optionally substituted 3-10 membered heterocyclyl containing 1, 2, 3, or 4 heteroatoms independently selected from nitrogen, oxygen, and sulfur.

In certain embodiments, L is -(3-7 membered, monocyclic, saturated heterocyclic ring containing 1 or 2 heteroatoms selected from nitrogen)-(OCH₂CH₂)₁₋₁₅—O—***, wherein *** is the point of attachment to A².

In certain embodiments, L is -(3-7 membered, monocyclic, saturated heterocyclic ring containing 1 or 2 heteroatoms selected from nitrogen)-(OCH₂CH₂)₁₋₅—O—***, wherein *** is the point of attachment to A².

In certain embodiments, L is -(3-7 membered, monocyclic, saturated heterocyclic ring containing 1 or 2 heteroatoms selected from nitrogen)-(OCH₂CH₂)₆₋₁₀—O—***, wherein *** is the point of attachment to A².

In certain embodiments, L is -piperidinylene-(OCH₂CH₂)₁₋₁₅—O—***, wherein *** is the point of attachment to A².

In certain embodiments, L is

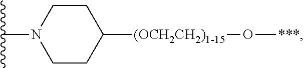

wherein *** is the point of attachment to A². In certain embodiments, L is

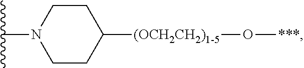

wherein *** is the point of attachment to A². In certain embodiments, L is

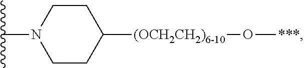

wherein *** is the point of attachment to A².

In certain embodiments, L is -(3-7 membered, monocyclic, saturated heterocyclic ring containing 1 or 2 heteroatoms selected from nitrogen)-(OCH₂CH₂)₁₋₁₀—N(H)C(O)—C₁₋₅ alkylene-***, -(3-7 membered, monocyclic, saturated heterocyclic ring containing 1 or 2 heteroatoms selected from nitrogen)-(OCH₂CH₂)₁₋₁₅—N(C₁₋₄ alkyl)C(O)—C₁₋₁₀ alkylene-***, -(3-7 membered, monocyclic, saturated heterocyclic ring containing 1 or 2 heteroatoms selected from nitrogen)-(OCH₂CH₂)₁₋₁₅—C(O)N(H)—C₁₋₁₀ alkylene-***, or -(3-7 membered, monocyclic, saturated heterocyclic ring containing 1 or 2 heteroatoms selected from nitrogen)-(OCH₂CH₂)₁₋₁₅—C(O)N(C₁₋₄ alkyl)-C₁₋₁₀ alkylene-***, wherein *** is the point of attachment to A².

In certain embodiments, L is -(3-7 membered, monocyclic, saturated heterocyclic ring containing 1 or 2 heteroatoms selected from nitrogen)-(OCH₂CH₂)₁₋₁₀—N(H)C(O)—C₁₋₅ alkylene-***, -(3-7 membered, monocyclic, saturated heterocyclic ring containing 1 or 2 heteroatoms selected from nitrogen)-(OCH₂CH₂)₁₋₁₀—N(C₁₋₄ alkyl)C(O)—C₁₋₅ alkylene-***, -(3-7 membered, monocyclic, saturated heterocyclic ring containing 1 or 2 heteroatoms selected from nitrogen)-(OCH₂CH₂)₁₋₁₀—C(O)N(H)—C₁₋₅ alkylene-***, or -(3-7 membered, monocyclic, saturated heterocyclic ring containing 1 or 2 heteroatoms selected from nitrogen)-OCH₂CH₂)₁₋₁₀—C(O)N(C₁₋₄ alkyl)-C₁₋₅ alkylene-***, wherein *** is the point of attachment to A².

In certain embodiments, L is -piperidinylene-(OCH₂CH₂)₁-s-N(H)C(O)—C₁₋₅ alkylene-***, -piperidinylene-(OCH₂CH₂)₁₋₅—N(C₁₋₄ alkyl)C(O)—C-5 alkylene-***, -piperidinylene-(OCH₂CH₂)₁₋₅—C(O)N(H)—C₁₋₅ alkylene-***, or -piperidinylene-(OCH₂CH₂)₁₋₅—C(O)N(C₁₋₄ alkyl)-C₁₋₅ alkylene-***, wherein *** is the point of attachment to A².

In certain embodiments, L is -(3-7 membered, monocyclic, saturated heterocyclic ring containing 1 or 2 heteroatoms selected from nitrogen)-(OCH₂CH₂)₁₋₁₀—***, -(3-7 membered, monocyclic, saturated heterocyclic ring containing 1 or 2 heteroatoms selected from nitrogen)-(C₀₋₁₀ alkylene)-O—***, or -(3-7 membered, monocyclic, saturated heterocyclic ring containing 1 or 2 heteroatoms selected from nitrogen)-C₁₋₁₀ alkylene, wherein *** is the point of attachment to A².

In certain embodiments, L is -piperidinylene-(OCH₂CH₂)₁₋₅—***, -piperidinylene-(C₀₋₅ alkylene)-O—***, or -piperidinylene-(C₁₋₅ alkylene)-***, wherein *** is the point of attachment to A².

In certain embodiments, L is -(3-7 membered monocyclic saturated heterocyclic ring containing 1 or 2 heteroatoms selected from nitrogen)-X¹—***, wherein *** is the point of attachment to A², and X¹ is (i) C₁₋₁₀ alkylene where 1 or 2 methylene groups are optionally replaced by —O—, —N(H)—, or —N(C₁₋₄ alkyl)-, (ii) a 3-7 membered monocyclic saturated heterocyclic ring containing 1 or 2 heteroatoms selected from nitrogen, or (iii) -(3-7 membered monocyclic saturated heterocyclic ring containing 1 or 2 heteroatoms selected from nitrogen)-(C₁₋₁₀ alkylene)-.

In certain embodiments, L is -(piperidinylene)-X¹—***, wherein *** is the point of attachment to A², and X¹ is (i) C₁₋₅ alkylene where 1 or 2 methylene groups are optionally replaced by —O—, —N(H)—, or —N(C₁₋₄ alkyl)-, (ii) a 3-4 membered monocyclic saturated heterocyclic ring containing 1 heteroatom selected from nitrogen, or (iii) -(3-4 membered monocyclic saturated heterocyclic ring containing 1 heteroatom selected from nitrogen)-(C₁₋₅ alkylene)-.

In certain embodiments, L is

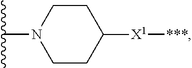

wherein *** is the point of attachment to A², and X¹ is (i) C₁₋₁₀ alkylene where 1 or 2 methylene groups are optionally replaced by —O—, —N(H)—, or —N(C₁₋₄ alkyl)-, (ii) a 3-7 membered monocyclic saturated heterocyclic ring containing 1 or 2 heteroatoms selected from nitrogen, or (iii) -(3-7 membered monocyclic saturated heterocyclic ring containing 1 or 2 heteroatoms selected from nitrogen)-(C₁₋₁₀ alkylene)-.

In certain embodiments, L is -(piperazinylene)-X¹—***, wherein *** is the point of attachment to A², and X¹ is (i) C₁₋₅ alkylene where 1 or 2 methylene groups are optionally replaced by —O—, (ii) a 3-4 membered monocyclic saturated heterocyclic ring containing 1 heteroatom selected from nitrogen, or (iii) -(3-4 membered monocyclic saturated heterocyclic ring containing 1 heteroatom selected from nitrogen)-(C₁₋₅ alkylene)-.

In certain embodiments, L is

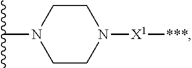

wherein *** is the point of attachment to A², and X¹ is (i) C₁₋₁₀ alkylene where 1 or 2 methylene groups are optionally replaced by —O—, (ii) a 3-7 membered monocyclic saturated heterocyclic ring containing 1 or 2 heteroatoms selected from nitrogen, or (iii) -(3-7 membered monocyclic saturated heterocyclic ring containing 1 or 2 heteroatoms selected from nitrogen)-(C₁₋₁₀ alkylene)-.

In certain embodiments, L is -(3-7 membered monocyclic saturated heterocyclic ring containing 1 or 2 heteroatoms selected from nitrogen)-X²—(C₁₋₁₀ alkylene)-***, wherein *** is the point of attachment to A², and X² is —O—, —N(H)—, or —N(C₁₋₆ alkyl)-. In certain embodiments, L is -(3-7 membered monocyclic saturated heterocyclic ring containing 1 or 2 heteroatoms selected from nitrogen)-X²—(C₁₋₁₀ alkylene)-***, wherein *** is the point of attachment to A², and X² is —O—.

In certain embodiments, L is -(piperidinylene)-X²—(C₁₋₁₀ alkylene)-***, wherein *** is the point of attachment to A², and X² is —O—, —N(H)—, or —N(C₁₋₆ alkyl)-. In certain embodiments, L is -(piperidinylene)-X²—(C₁₋₁₀ alkylene)-***, wherein *** is the point of attachment to A², and X² is —O—.

In certain embodiments, L is -(piperidinylene)-X²-(a 3-7 membered monocyclic saturated heterocyclic ring containing 1 or 2 heteroatoms selected from nitrogen)-***, wherein *** is the point of attachment to A², and X² is —O—, —N(H)—, or —N(C₁₋₆ alkyl)-. In certain embodiments, L is -(piperidinylene)-X²-(a 3-7 membered monocyclic saturated heterocyclic ring containing 1 or 2 heteroatoms selected from nitrogen)-***, wherein *** is the point of attachment to A², and X² is —O—.

In certain embodiments, L is

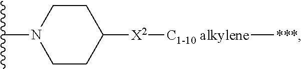

wherein *** is the point of attachment to A², and X² is —O—, —N(H)—, or —N(C₁₋₆ alkyl)-. In certain embodiments, L is

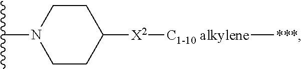

wherein *** is the point of attachment to A², and X² is —O—.

In certain embodiments, L is 3-7 membered monocyclic saturated

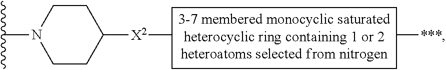

wherein *** is the point of attachment to A², and X² is —O—, —N(H)—, or —N(C₁₋₆ alkyl)-. In certain embodiments, L is

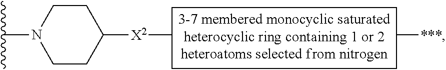

wherein *** is the point of attachment to A², and X² is —O—.

In certain embodiments, L is -(3-7 membered monocyclic saturated heterocyclic ring containing 1 or 2 heteroatoms selected from nitrogen)-X¹—***, wherein *** is the point of attachment to A², and X¹ is —(OCH₂CH₂)₁₋₁₀ where 1 CH₂ group is optionally replaced with —C(H)(C₃₋₆ cycloalkyl)-.

In certain embodiments, L is a 7-11 membered spirocyclic or fused bicyclic saturated heterocyclic ring containing 1, 2, or 3 heteroatoms selected from nitrogen and oxygen. In certain embodiments, L is a 7-8 membered spirocyclic or fused bicyclic saturated heterocyclic ring containing 2 heteroatoms selected from nitrogen.

In certain embodiments, L is -(3-7 membered monocyclic saturated heterocyclic ring containing 1 or 2 heteroatoms selected from nitrogen)-X³-(3-7 membered monocyclic saturated heterocyclic ring containing 1 or 2 heteroatoms selected from nitrogen)-, wherein X³ is C₁₋₁₀ alkylene, —O—, —N(H)—, —N(C₁₋₄ alkyl)-, or a bond.

In certain embodiments, L is -(piperidinylene)-(C₁₋₅ alkylene)-(piperazinylene)-*** wherein *** is the point of attachment to A².

In certain embodiments, L is -(piperazinylene)-(azetidinylene)-*** or (azetidinylene)-(piperazinylene)-***, wherein *** is the point of attachment to A².

In certain embodiments, L is -(piperidinylene)-X³-(azetidinylene)- or -(azetidinylene)-X³-(azetidinylene)-, wherein X³ is C₁₋₃ alkylene, and *** is the point of attachment to A².

In certain embodiments, L is -(3-7 membered monocyclic saturated heterocyclic ring containing 1 or 2 heteroatoms selected from nitrogen)-X³—(C₃₋₆ cycloalkylene)-O—***, -(3-7 membered monocyclic saturated heterocyclic ring containing 1 or 2 heteroatoms selected from nitrogen)-X³—(C₃₋₆ cycloalkylene)-N(H)—***, or -(3-7 membered monocyclic saturated heterocyclic ring containing 1 or 2 heteroatoms selected from nitrogen)-X³—(C₃₋₆ cycloalkylene)-N(C₁₋₄ alkyl)-***, wherein *** is the point of attachment to A², and X³ is C₁₋₁₀ alkylene, —O—, —N(H)—, —N(C₁₋₄ alkyl)-, or a bond.

In certain embodiments, L is -(piperidinylene)-X³—(C₃₋₆ cycloalkylene)-O—***, -(piperidinylene)-X³—(C₃₋₆ cycloalkylene)-N(H)—***, or -(piperidinylene)-X³—(C₃₋₆ cycloalkylene)-***, wherein *** is the point of attachment to A², and X³ is C₁₋₁₀ alkylene, —O—, —N(H)—, —N(C₁₋₄ alkyl)-, or a bond.

In certain embodiments, L is —N(C₁₋₃ alkyl)-(C₂₋₇ alkylene)-O—***, —N(H)—(C₂₋₇ alkylene)-O—***, —N(C₁₋₃ alkyl)-[(C₂₋₄ alkylene)-O—]₂₋₁₀-***, or —N(H)—[(C₂₋₄ alkylene)-O—]₂₋₁₀—***, where *** is a point of attachment to A².

In certain embodiments, L is —N(C₁₋₃ alkyl)-(C₁₋₆ alkylene)-(5-6 membered saturated heterocyclylene containing 1 or 2 heteratoms independently selected from nitrogen and oxygen)-(C₁₋₆ alkylene)-O—*** or —N(H)—(C₁₋₆ alkylene)-(5-6 membered saturated heterocyclylene containing 1 or 2 heteratoms independently selected from nitrogen and oxygen)-(C₁₋₆ alkylene)-0-***, where *** is a point of attachment to A².

In certain embodiments, L is —N(C₁₋₃ alkyl)-(C₂₋₇ alkylene)-N(R)—*** or —N(H)—(C₂₋₇ alkylene)-N(R)—***, where *** is a point of attachment to A², and R is hydrogen or C₁₋₃ alkyl.

In certain embodiments, L is —N(C₁₋₃ alkyl)-(C₁₋₆ alkylene)-(5-6 membered saturated heterocyclylene containing 1 or 2 heteratoms independently selected from nitrogen and oxygen)-(C₁₋₆ alkylene)-N(R)—***, —N(H)—(C₁₋₆ alkylene)-(5-6 membered saturated heterocyclylene containing 1 or 2 heteratoms independently selected from nitrogen and oxygen)-(C₁₋₆ alkylene)-N(R)—***, where *** is a point of attachment to A², and R is hydrogen or C₁₋₃ alkyl.

In certain embodiments, L is —N(C₁₋₃ alkyl)-(C₂₋₇ alkylene)-N(H)C(O)—***, —N(H)—(C₂₋₇ alkylene)-N(H)C(O)—***, —N(H)—(C₂₋₇ alkylene)-N(C₁₋₃ alkyl)C(O)—***, —N(C₁₋₃ alkyl)-(C₂₋₇ alkylene)-N(C₁₋₃ alkyl)C(O)—***, —N(C₁₋₃ alkyl)-(C₂₋₇ alkylene)-C(O)N(H)—***, —N(H)—(C₂₋₇ alkylene)-C(O)N(H)—***, —N(H)—(C₂₋₇ alkylene)-C(O)N(C₁₋₃ alkyl)-***, —N(C₁₋₃ alkyl)-(C₂₋₇ alkylene)-C(O)N(C₁₋₃ alkyl)-***, —N(C₁₋₃ alkyl)-[(C₂₋₄ alkylene)-O—]₂₋₈—(C₂₋₆ alkylene)-N(H)C(O)—***, —N(H)—[(C₂₋₄ alkylene)-O—]₂₋₈—(C₂₋₆ alkylene)-N(H)C(O)—***, —N(H)—[(C₂₋₄ alkylene)-O—]₂₋₈—(C₂₋₆ alkylene)-N(C₁₋₃ alkyl)C(O)—***, —N(C₁₋₃ alkyl)-[(C₂₋₄ alkylene)-O—]₂₋₈—(C₂₋₆ alkylene)-N(C_{1—. 3} alkyl)C(O)—***, —N(C₁₋₃ alkyl)-[(C₂₋₄ alkylene)-O—]₂₋₈—(C₂₋₆ alkylene)-C(O)N(H)—***, —N(H)—[(C₂₋₄ alkylene)-O—]₂₋₈—(C₂₋₆ alkylene)-C(O)N(H)—***, —N(H)—[(C₂₋₄ alkylene)-O—]₂₋₈—(C₂₋₆ alkylene)-C(O)N(C₁₋₃ alkyl)-***, or —N(C₁₋₃ alkyl)-[(C₂₋₄ alkylene)-O—]₂₋₈—(C₂₋₆ alkylene)-C(O)N(C₁₋₃ alkyl)-***, where *** is a point of attachment to A².

In certain embodiments, L is —N(C₁₋₃ alkyl)-(C₁₋₆ alkylene)-(5-6 membered saturated heterocyclylene containing 1 or 2 heteratoms independently selected from nitrogen and oxygen)-(C₁₋₆ alkylene)-N(H)C(O)—***, —N(C₁₋₃ alkyl)-(C₁₋₆ alkylene)-(5-6 membered saturated heterocyclylene containing 1 or 2 heteratoms independently selected from nitrogen and oxygen)-(C₁₋₆ alkylene)-N(C₁₋₃ alkyl)C(O)—***, —N(H)—(C₁₋₆ alkylene)-(5-6 membered saturated heterocyclylene containing 1 or 2 heteratoms independently selected from nitrogen and oxygen)-(C₁₋₆ alkylene)-N(C₁₋₃ alkyl)C(O)—***, —N(H)—(C₁₋₆ alkylene)-(5-6 membered saturated heterocyclylene containing 1 or 2 heteratoms independently selected from nitrogen and oxygen)-(C₁₋₆ alkylene)-N(H)C(O)—***, —N(C₁₋₃ alkyl)-(C₁₋₆ alkylene)-(5-6 membered saturated heterocyclylene containing 1 or 2 heteratoms independently selected from nitrogen and oxygen)-(C₁₋₆ alkylene)-C(O)N(H)—***, —N(C₁₋₃ alkyl)-(C₁₋₆ alkylene)-(5-6 membered saturated heterocyclylene containing 1 or 2 heteratoms independently selected from nitrogen and oxygen)-(C₁₋₆ alkylene)-C(O)N(C₁₋₃ alkyl)-***, —N(H)—(C₁₋₆ alkylene)-(5-6 membered saturated heterocyclylene containing 1 or 2 heteratoms independently selected from nitrogen and oxygen)-(C₁₋₆ alkylene)-C(O)N(C₁₋₃ alkyl)-***, or —N(H)—(C₁₋₆ alkylene)-(5-6 membered saturated heterocyclylene containing 1 or 2 heteratoms independently selected from nitrogen and oxygen)-(C₁₋₆ alkylene)-C(O)N(H)—***, where *** is a point of attachment to A².

In certain embodiments, L has the formula —(C₀₋₁₂ alkylene)-(optionally substituted 3-40 membered heteroalkylene)-(C₀₋₁₂ alkylene)-.

In certain embodiments, L is one of the following:

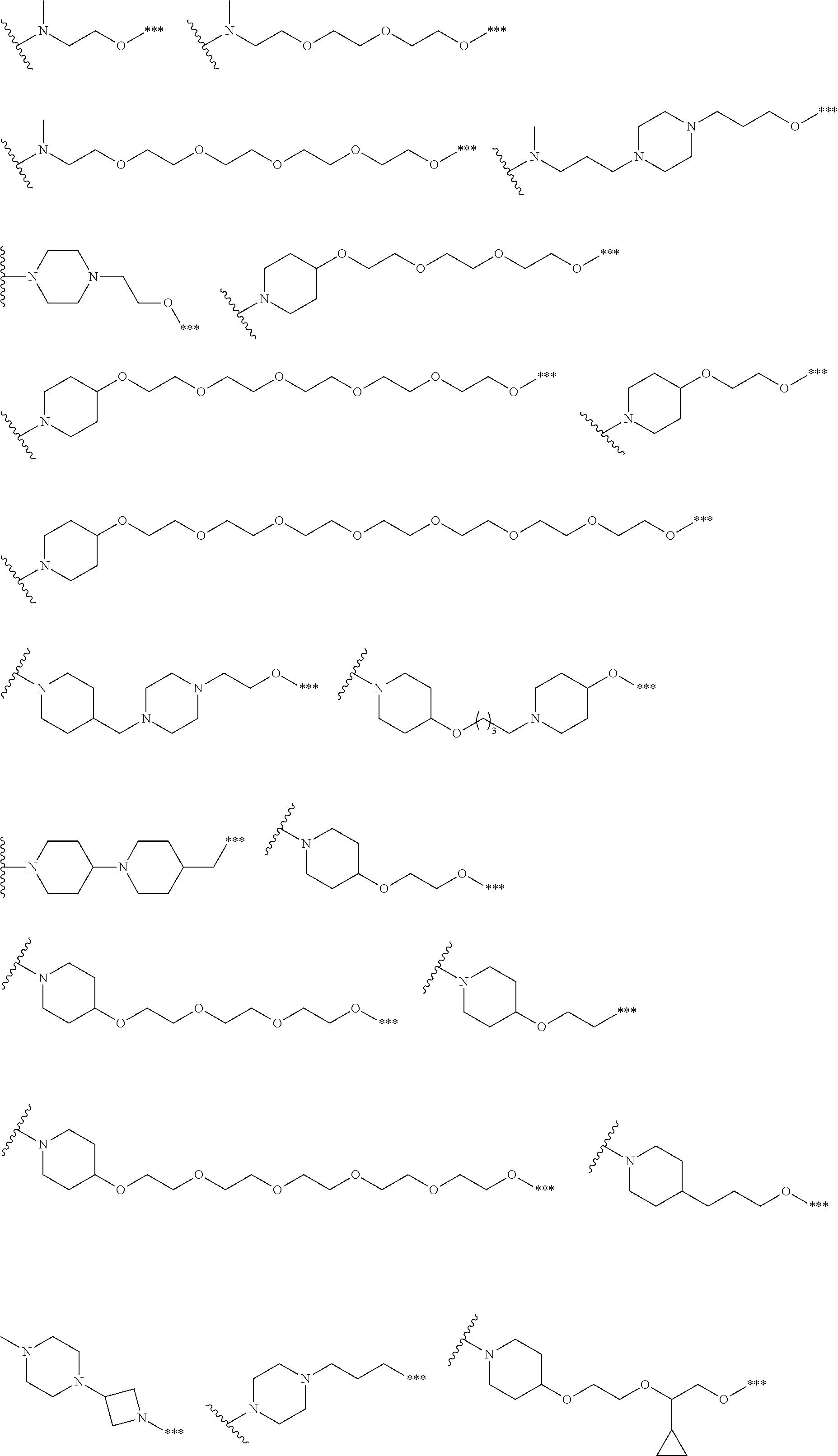

wherein *** is the point of attachment to A².

In certain embodiments, L is one of the following:

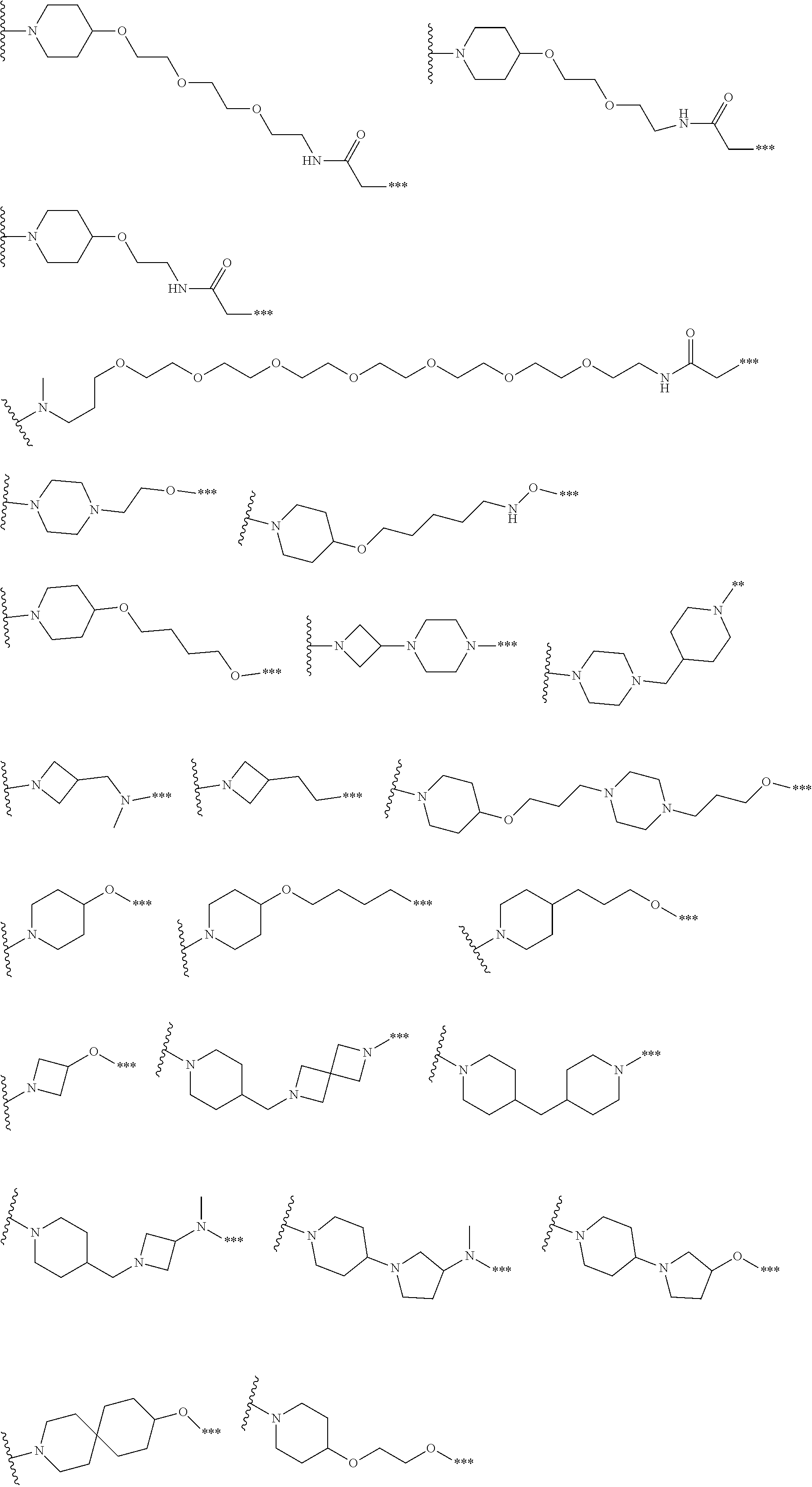

wherein *** is the point of attachment to A².

In certain embodiments, L is one of the following:

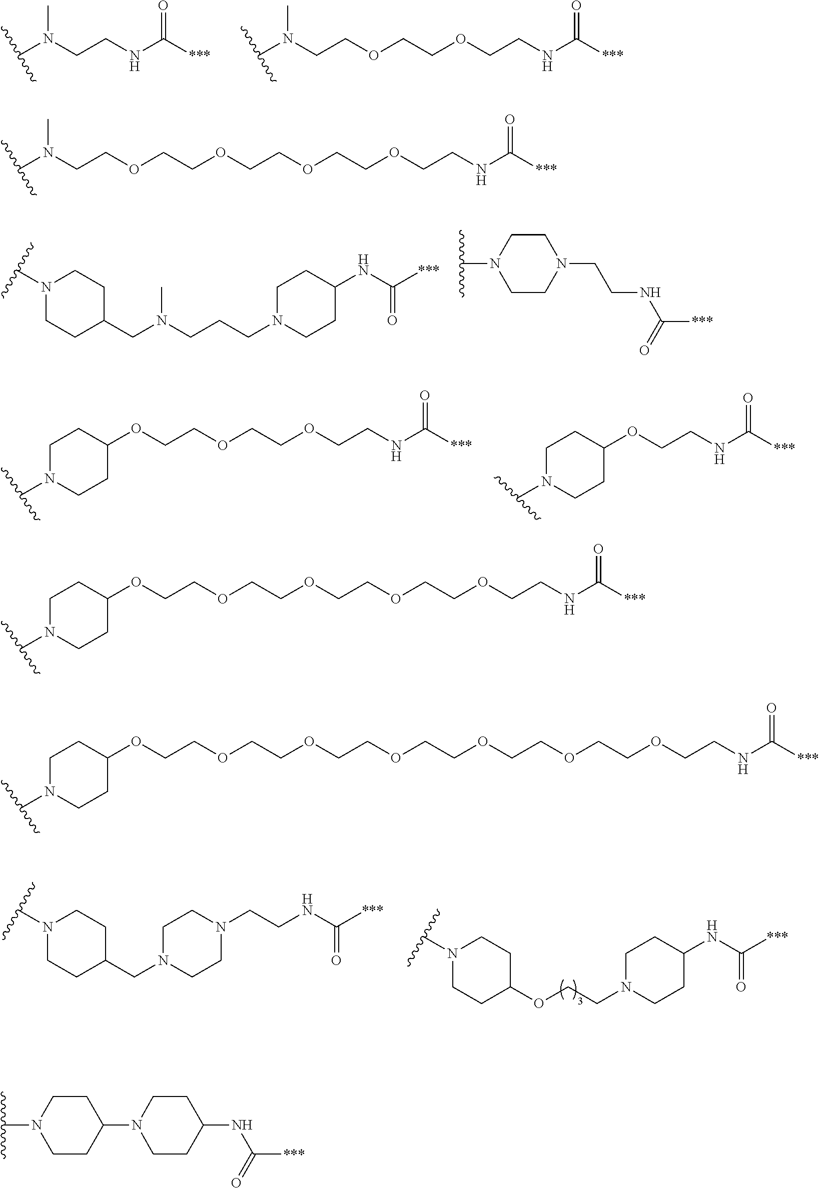

wherein *** is the point of attachment to A².

In certain embodiments, L is one of the following:

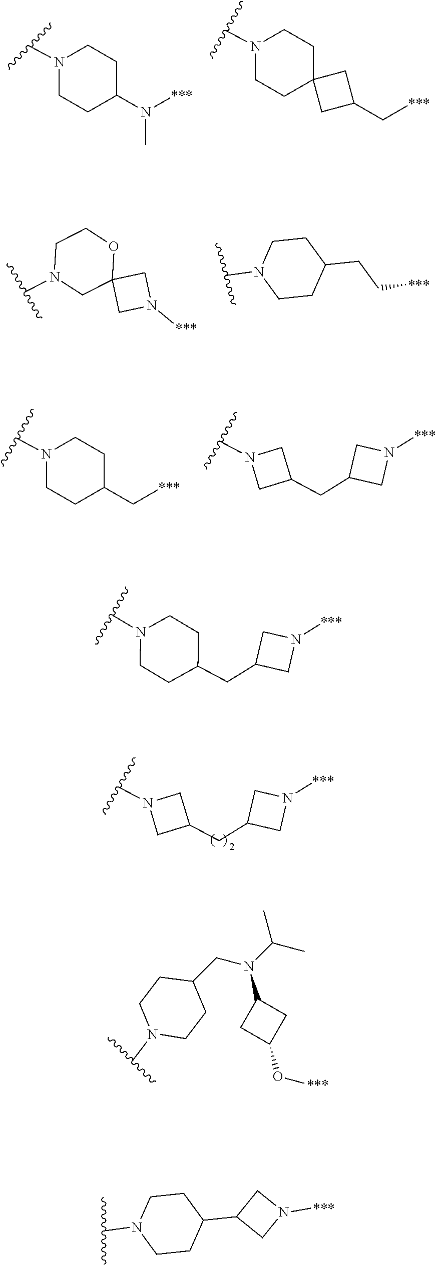

wherein *** is the point of attachment to A².

In certain embodiments, L is -(3-7 membered, monocyclic, saturated heterocyclic ring containing 1 or 2 heteroatoms selected from nitrogen)-O—(C₁₋₆ alkylene)-(3-7 membered, monocyclic, saturated heterocyclic ring containing 1 or 2 heteroatoms selected from nitrogen)-(C₁₋₆ alkylene)-O—***, -(3-7 membered, monocyclic, saturated heterocyclic ring containing 1 or 2 heteroatoms selected from nitrogen)-(C₁₋₆ alkylene)-(C₁₋₆ alkylene)-(3-7 membered, monocyclic, saturated heterocyclic ring containing 1 or 2 heteroatoms selected from nitrogen)-(C₁₋₆ alkylene)-O—***, -(3-7 membered, monocyclic, saturated heterocyclic ring containing 1 or 2 heteroatoms selected from nitrogen)-(C₁₋₆ alkylene)-(C₀₋₆ alkylene)-(7-9 membered spirocyclic saturated heterocyclic ring containing 1 or 2 heteroatoms selected from nitrogen)-(C₀₋₆ alkylene)-***, -(7-12 membered spirocyclic saturated heterocyclic ring containing 1 or 2 heteroatoms selected from nitrogen)-(C₀₋₆ alkylene)-O—(C₀₋₆ alkylene)-***, -(7-12 membered spirocyclic saturated heterocyclic ring containing 1 or 2 heteroatoms selected from nitrogen)-(C₁₋₆ alkylene)-***, -(7-12 membered spirocyclic saturated heterocyclic ring containing 1 or 2 heteroatoms selected from nitrogen)-(C₃₋₆ cycloalkylene)-***, -(7-12 membered spirocyclic saturated heterocyclic ring containing 1 or 2 heteroatoms selected from nitrogen)-(3-7 membered, monocyclic, saturated heterocyclic ring containing 1 or 2 heteroatoms selected from nitrogen)-***, -(7-12 membered spirocyclic saturated heterocyclic ring containing 1 or 2 heteroatoms selected from nitrogen)-(C₂₋₄ alkynylene)-***, -(3-7 membered, monocyclic, saturated heterocyclic ring containing 1 or 2 heteroatoms selected from nitrogen)-O—(C₁₋₆ alkylene)-N(C₁₋₆ alkyl)C(O)—***, -(7-10 membered, fused bicyclic saturated heterocyclic ring containing 1 or 2 heteroatoms selected from nitrogen)-(C₁₋₆ alkylene)-***, -(7-10 membered, fused bicyclic saturated heterocyclic ring containing 1 or 2 heteroatoms selected from nitrogen)-(C₁₋₆ alkylene)-(3-7 membered, monocyclic, saturated heterocyclic ring containing 1 or 2 heteroatoms selected from nitrogen)-***, -(3-7 membered, monocyclic, saturated heterocyclic ring containing 1 or 2 heteroatoms selected from nitrogen)-(C₃₋₆ cycloalkylene)-N(C₁₋₆ alkyl)-(C₀₋₆ alkylene)-***, -(3-7 membered, monocyclic, saturated heterocyclic ring containing 1 or 2 heteroatoms selected from nitrogen)-N(C₁₋₆ alkyl)-(C₁₋₆ alkylene)-N(H)—(C₀₋₆ alkylene)-***, -(3-7 membered, monocyclic, saturated heterocyclic ring containing 1 or 2 heteroatoms selected from nitrogen)-N(C₁₋₆ alkyl)-(C₁₋₆ alkylene)-N(C₁₋₆ alkylene)-(C₀₋₆ alkylene)-***, -(3-7 membered, monocyclic, saturated heterocyclic ring containing 1 or 2 heteroatoms selected from nitrogen)-(C₁₋₆ alkylene)-N(C₁₋₆ alkyl)-(C₃₋₆ cycloalkylene)-O—(C₀₋₆ alkylene)-*** -(3-7 membered, monocyclic, saturated heterocyclic ring containing 1 or 2 heteroatoms selected from nitrogen)-N(C₃₋₆ cycloalkyl)-(3-7 membered, monocyclic, saturated heterocyclic ring containing 1 or 2 heteroatoms selected from nitrogen)-***, -(5-9 membered, bridged bicyclic, saturated heterocyclic ring containing 1 or 2 heteroatoms selected from nitrogen)-(C₁₋₆ alkylene)-(3-7 membered, monocyclic, saturated heterocyclic ring containing 1 or 2 heteroatoms selected from nitrogen)-***, -(3-7 membered monocyclic saturated heterocyclic ring containing 1 or 2 heteroatoms selected from nitrogen)-O—(3-7 membered monocyclic saturated heterocyclic ring containing 1 or 2 heteroatoms selected from nitrogen)-(C₁₋₆ alkylene)-O—***, -(7-10 membered bridged bicyclic saturated heterocyclic ring containing 1 or 2 heteroatoms selected from nitrogen)-(C₁₋₆ alkylene)-(3-7 membered monocyclic saturated heterocyclic ring containing 1 or 2 heteroatoms selected from nitrogen)-(C₀₋₆ alkylene)-***, -(3-7 membered monocyclic saturated heterocyclic ring containing 1 or 2 heteroatoms selected from nitrogen)-(C₀₋₆ alkylene)-(7-11 membered spirocyclic saturated heterocyclic ring containing 1 or 2 heteroatoms selected from nitrogen)-O—(C₀₋₆ alkylene)-***, -(6-10 membered bicyclic saturated heterocyclic ring containing 1 or 2 heteroatoms selected from nitrogen)-(C₁₋₆ alkylene)-***, -(4-6 membered monocyclic heterocyclyl containing 1 or 2 heteroatoms selected from nitrogen)-(C₀₋₆ alkylene)-O—(5-6 membered heteroaryl containing 1 or 2 heteroatoms selected from nitrogen)-O—***, -(4-6 membered monocyclic heterocyclyl containing 1 or 2 heteroatoms selected from nitrogen)-(C₀₋₆ alkylene)-(4-6 membered monocyclic heterocyclyl containing 1 or 2 heteroatoms selected from nitrogen)-O—***, or -(4-6 membered monocyclic heterocyclyl containing 1 or 2 heteroatoms selected from nitrogen)-(C₃₋₆ cycloalkylene)-(C₀₋₆ alkylene)-***, wherein *** is the point of attachment to A².

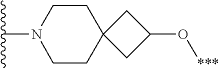

In certain embodiments, L is *** wherein *** is the point of attachment to A². In certain embodiments, L is

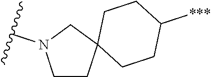

wherein *** is the point of attachment to A².

### Additional Exemplary Embodiments for L

In certain embodiments, L is —N(H)—(C₂₋₉ alkylene)-O—(C₁₋₆ alkylene)-C(O)—***, —N(H)—(C₁₋₂₀ alkylene)-O—(C₁₋₆ alkylene)-C(O)—***, —N(H)—[(C₂₋₄ alkylene)-O—]₂₋₆—(C₁₋₆ alkylene)-C(O)—***, —N(H)—[(C₂₋₄ alkylene)-O—]₇₋₁₅—(C₁₋₆ alkylene)-C(O)—***, —N(H)—(C₁₋₆ alkylene)-C(O)—***, —N(H)—(C₇₋₁₅ alkylene)-C(O)—***, —N(H)—[(C₂₋₄ alkylene)-O—]₂₋₆—(C₁₋₆ alkylene)-***, —N(H)—[(C₂₋₄ alkylene)-O—]₇₋₁₅—(C₁₋₆ alkylene)-***, —N(H)—(C₂₋₉ alkylene)-O—(C₁₋₆ alkylene)-C(O)N(C₁₋₆ alkyl)-(C₁₋₆ alkylene)-***, —N(H)—(C₂₋₉ alkylene)-O—(C₁₋₆ alkylene)-C(O)N(H)—(C₁₋₆ alkylene)-***, —N(H)—[(C₂₋₄ alkylene)-O—]₂₋₆—(C₁₋₆ alkylene)-N(H)—(C₁₋₆ alkylene)-***, —N(H)—[(C₂₋₄ alkylene)-O—]₇₋₁₅—(C₁₋₆ alkylene)-N(H)—(C₁₋₆ alkylene)-***, —N(H)—[(C₂₋₄ alkylene)-O—]₂₋₆—(C₁₋₆ alkylene)-N(C₁₋₆ alkyl)-(C₁₋₆ alkylene)-***, or —N(H)—[(C₂₋₄ alkylene)-O—]₇₋₁₅—(C₁₋₆ alkylene)-N(C₁₋₆ alkyl)-(C₁₋₆ alkylene)-***, where *** is a point of attachment to A².

In certain embodiments, L is —N(H)—(C₂₋₉ alkylene)-O—(C₁₋₆ alkylene)-C(O)—***, —N(H)—(C₁₀₋₂₀ alkylene)-O—(C₁₋₆ alkylene)-C(O)—***, —N(H)—[CH₂CH₂—O—]₂₋₆—(C₁₋₆ alkylene)-C(O)—***, —N(H)—[CH₂CH₂—O—]₇₋₁₆—(C₁₋₆ alkylene)-C(O)—***, —N(H)—(C₁₋₆ alkylene)-C(O)—***, —N(H)—(C₇₋₁₅ alkylene)-C(O)—***, —N(H)—[CH₂CH₂—O—]₂₋₆—(C₁₋₆ alkylene)-***, —N(H)—[CH₂CH₂—O—]₇₋₁₅—(C₁₋₆ alkylene)-***, —N(H)—(C₂₋₉ alkylene)-O—(C₁₋₆ alkylene)-C(O)N(C₁₋₆ alkyl)-(C₁₋₆ alkylene)-***, —N(H)—(C₂₋₉ alkylene)-O—(C₁₋₆ alkylene)-C(O)N(H)—(C₁₋₆ alkylene)-***, —N(H)—[CH₂CH₂—O-]₂₋₆—(C₁₋₆ alkylene)-N(H)—(C₁₋₆ alkylene)-***, —N(H)—[CH₂CH₂—O—]₇₋₁₅—(C₁₋₆ alkylene)-N(H)—(C₁₋₆ alkylene)-***, —N(H)—[CH₂CH₂—O—]₂₋₆—(C₁₋₆ alkylene)-N(C₁₋₆ alkyl)-(C₁₋₆ alkylene)-***, or —N(H)—[CH₂CH₂—O—]₁₋₁₅—(C₁₋₆ alkylene)-N(C₁₋₆ alkyl)-(C₁₋₆ alkylene)-***, where *** is a point of attachment to A².

In certain embodiments, L is —N(H)—[(C₂₋₄ alkylene)-O—]₂₋₆—(C₁₋₆ alkylene)-C(O)—***, —N(H)—[(C₂₋₄ alkylene)-O—]₇₋₁₅—(C₁₋₆ alkylene)-C(O)—***, —N(H)—(C₁₋₆ alkylene)-N(C₁₋₆ alkyl)C(O)—(C₁₋₆ alkylene)***, —N(H)—(C₁₋₆ alkylene)-N(H)C(O)—(C₁₋₆ alkylene)***, —N(H)—(C₂₋₆ alkylene)-***, —N(H)—(C₇₋₁₅ alkylene)-***, —N(C₁₋₆ alkyl)-(C₂₋₆ alkylene)-***, —N(C₁₋₆ alkyl)-(C₇₋₁₅ alkylene)-***, —N(H)—[(C₂₋₄ alkylene)-O—]₂₋₆—(C₁₋₆ alkylene)-***, —N(H)—[(C₂₋₄ alkylene)-O—]₇₋₁₅—(C₁₋₆ alkylene)-***, —N(H)—(C₁₋₆ alkylene)-(3-6 membered heterocycloalkylene)-(C₁₋₆ alkylene)-N(C₁₋₆ alkyl)-(C₁₋₆ alkylene)-***, —N(H)—(C₁₋₆ alkylene)-(3-6 membered heterocycloalkylene)-(C₁₋₆ alkylene)-N(H)—(C₁₋₆ alkylene)-***, —N(H)—(C₂₋₆ alkylene)-N(H)—(C₁₋₆ alkylene)-***, or —N(H)—(C₂₋₆ alkylene)-N(C₁₋₆ alkyl)-(C₁₋₆ alkylene)-***, where *** is a point of attachment to A².

In certain embodiments, L is —N(H)—[CH₂CH₂—O—]₂₋₆—(C₁₋₆ alkylene)-C(O)—***, —N(H)—[CH₂CH₂—O—]₇₋₁₅—(C₁₋₆ alkylene)-C(O)—***, —N(H)—(C₁₋₆ alkylene)-N(C₁₋₆ alkyl)C(O)—(C₁₋₆ alkylene)***, —N(H)—(C₁₋₆ alkylene)-N(H)C(O)—(C₁₋₆ alkylene)***, —N(H)—(C₂₋₆ alkylene)-***, —N(H)—(C₇₋₁₅ alkylene)-***, —N(C₁₋₆ alkyl)-(C₂₋₆ alkylene)-***, —N(C₁₋₆ alkyl)-(C₇₋₁₅ alkylene)-***, —N(H)—[CH₂CH₂—O—]₂₋₆—(C₁alkylene)-***, —N(H)—[CH₂CH₂—O—]₇₋₁₅—(C₁₋₆ alkylene)-***, —N(H)—(C₁₋₆ alkylene)-(3-6 membered heterocycloalkylene)-(C₁₋₆ alkylene)-N(C₁₋₆ alkyl)-(C₁₋₆ alkylene)-***, —N(H)—(C₁₋₆ alkylene)-(3-6 membered heterocycloalkylene)-(C₁₋₆ alkylene)-N(H)—(C₁₋₆ alkylene)-***, —N(H)—(C₂₋₆ alkylene)-N(H)—(C₁₋₆ alkylene)-***, or —N(H)—(C₂₋₆ alkylene)-N(C₁₋₆ alkyl)-(C₁₋₆ alkylene)-***, where *** is a point of attachment to A².

In certain embodiments, L is —[(C₂₋₄ alkylene)-O—]₂₋₆-(CL-6 alkylene)-***, —[(C₂₋₄ alkylene)-O—]₇₋₁₅—(C₁₋₆ alkylene)-***, —[(C₂₋₄ alkylene)-O—]₂₋₆—(C₁₋₆ alkylene)-N(C₁₋₆ alkyl)(C₁₋₆ alkylene)-***, —[(C₂₋₄ alkylene)-O—]₇₋₁i-(C₁₋₆ alkylene)-N(C₁₋₆ alkyl)(C₁₋₆ alkylene)-***, —[(C₂₋₄ alkylene)-O—]₂₋₆—(C₁₋₆ alkylene)-N(H)(C₁₋₆ alkylene)-***, —[(C₂₋₄ alkylene)-O—]₇₋₁₅—(C₁₋₆ alkylene)-N(H)(C₁₋₆ alkylene)-***, —(C₁₋₉ alkylene)-C(O)N(H)—(C₁₋₆ alkylene)-***, —(C₁₋₉ alkylene)-N(H)C(O)—(C₁₋₆ alkylene)-***, —(C₁₋₉ alkylene)-C(O)N(H)—[(C₂₋₄ alkylene)-O—]₂₋₆—(C₁₋₆ alkylene)-***, —(C₁₋₉ alkylene)-N(H)C(O)—[(C₂₋₄ alkylene)-O—]₂₋₆—(C₁₋₆ alkylene)-***, —(C₁₋₉ alkylene)-C(O)N(H)—[(C₂₋₄ alkylene)-O—]₅₋₁₅—(C₁₋₆ alkylene)-***, —(C₁₋₉ alkylene)-N(H)C(O)—[(C₂₋₄ alkylene)-O—]₇₋₁₅—(C₁₋₆ alkylene)-***, —(C₁₋₉ alkylene)-C(O)N(H)—[(C₂₋₄ alkylene)-O—]₂₋₆—(C₁₋₆ alkylene)-N(C₁₋₆ alkyl)-(C₁₋₆ alkylene)-***, —(C₁₋₉ alkylene)-N(H)C(O)—[(C₂₋₄ alkylene)-O—]₂₋₆—(C₁₋₆ alkylene)-N(C₁₋₆ alkyl)-(C₁₋₆ alkylene)-***, —(C₁₋₉ alkylene)-C(O)N(H)—[(C₂₋₄ alkylene)-O-]₇₋₁₅—(C₁₋₆ alkylene)-N(C₁₋₆ alkyl)-(C₁₋₆ alkylene)-***, or —(C₁₋₉ alkylene)-N(H)C(O)—[(C₂₋₄ alkylene)-O—]₇₋₁₅—(C₁₋₆ alkylene)-N(C₁₋₆ alkyl)-(C₁₋₆ alkylene)-***, where *** is a point of attachment to A².

In certain embodiments, L is —[CH₂CH₂—O—]₂₋₆—(C₁₋₆ alkylene)-***, —[CH₂CH₂—O—]₇₋₁₅—(C₁₋₆ alkylene)-***, —[CH₂CH₂—O—]₂₋₆—(C₁₋₆ alkylene)-N(C₁₋₆ alkyl)(C₁₋₆ alkylene)-***, —[CH₂CH₂—O—]₇₋₁₅—(C₁₋₆ alkylene)-N(C₁₋₆ alkyl)(C₁₋₆ alkylene)-***, —[CH₂CH₂—O—]₂₋₆—(C₁₋₆ alkylene)-N(H)(C₁₋₆ alkylene)-***, —[CH₂CH₂—O—]₇₋₁₅—(C₁₋₆ alkylene)-N(H)(C₁₋₆ alkylene)-***, —(C₁₋₉ alkylene)-C(O)N(H)—(C₁₋₆ alkylene)-***, —(C₁₋₉ alkylene)-N(H)C(O)—(C₁₋₆ alkylene)-***, —(C₁₋₉ alkylene)-C(O)N(H)—[CH₂CH₂—O—]₂₋₆—(C₁₋₆ alkylene)-***, —(C₁₋₉ alkylene)-N(H)C(O)—[CH₂CH₂—O —]₂₋₆—(C₁₋₆ alkylene)-***, —(C₁₋₉ alkylene)-C(O)N(H)—[CH₂CH₂—O—]₇₋₁₅—(C₁₋₆ alkylene)-***, —(C₁₋₉ alkylene)-N(H)C(O)—[CH₂CH₂—O—]₇₋₁₅—(C₁₋₆ alkylene)-***, —(C₁₋₉ alkylene)-C(O)N(H)—[CH₂CH₂—O—]₂₋₆—(C₁₋₆ alkylene)-N(C₁₋₆ alkyl)-(C₁₋₆ alkylene)-***, —(C₁₋₉ alkylene)-N(H)C(O)—[CH₂CH₂—O—]₂₋₆—(C₁₋₆ alkylene)-N(C₁₋₆ alkyl)-(C₁₋₆ alkylene)-***, —(C₁₋₉ alkylene)-C(O)N(H)—[CH₂CH₂—O—]₇₋₁₅—(C₁₋₆ alkylene)-N(C₁₋₆ alkyl)-(C₁₋₆ alkylene)-***, or —(C₁₋₉ alkylene)-N(H)C(O)—[(CH₂CH₂—O—]₇₋₁₅—(C₁₋₆ alkylene)-N(C₁₋₆ alkyl)-(C₁₋₆ alkylene)-***, where *** is a point of attachment to A².

In certain embodiments, L is —N(H)—[(C₂₋₄ alkylene)-O—]₂₋₆—(C₁₋₆ alkylene)-N(H)—***, —N(H)—[(C₂₋₄ alkylene)-O—]₇₋₁₅—(C₁₋₆ alkylene)-N(H)—***, —N(C₁₋₆ alkyl)-[(C₂₋₄ alkylene)-O—]₂₋₆—(C₁-₆ alkylene)-N(H)—***, —N(C₁₋₆ alkyl)-[(C₂₋₄ alkylene)-O—]₇₋₁₅—(C₁₋₆ alkylene)-N(H)—***, —N(C₁₋₆ alkyl)-[(C₂₋₄ alkylene)-O—]₂₋₆—(C₁₋₆ alkylene)-N(C₁₋₆ alkyl)-***, or —N(C₁₋₆ alkyl)-[(C₂₋₄ alkylene)-O—]₇₋₁₅—(C₁₋₆ alkylene)-N(C₁₋₆ alkyl)-***, where *** is a point of attachment to A².

In certain embodiments, L is —N(H)—[CH₂CH₂—O—]₂₋₆—(C₁₋₆ alkylene)-N(H)—***, —N(H)—[CH₂CH₂—O—]₇₋₁i-(C₁₋₆ alkylene)-N(H)—***, —N(C₁₋₆ alkyl)-[CH₂CH₂—O—]₂₋₆—(C₁₋₆ alkylene)-N(H)—***, —N(C₁₋₆ alkyl)-[CH₂CH₂—O—]₇₋₁₅—(C₁₋₆ alkylene)-N(H)—***, —N(C₁₋₆ alkyl)-[CH₂CH₂—O—]₂₋₆—(C₁₋₆ alkylene)-N(C₁₋₆ alkyl)-***, or —N(C₁₋₆ alkyl)-[CH₂CH₂—O—]₇₋₁₅—(C₁₋₆ alkylene)-N(C₁₋₆ alkyl)-***, where *** is a point of attachment to A².

In some embodiments, L is one of the following:

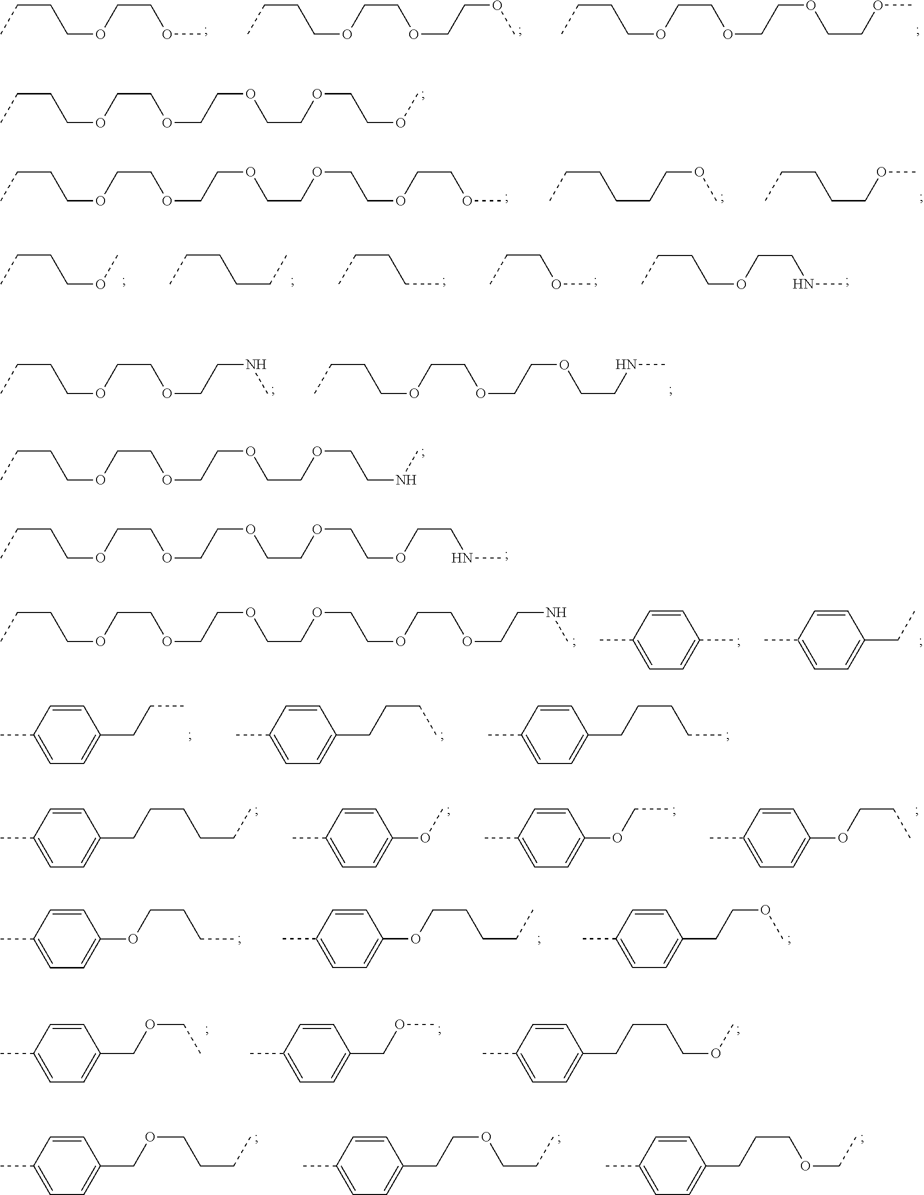

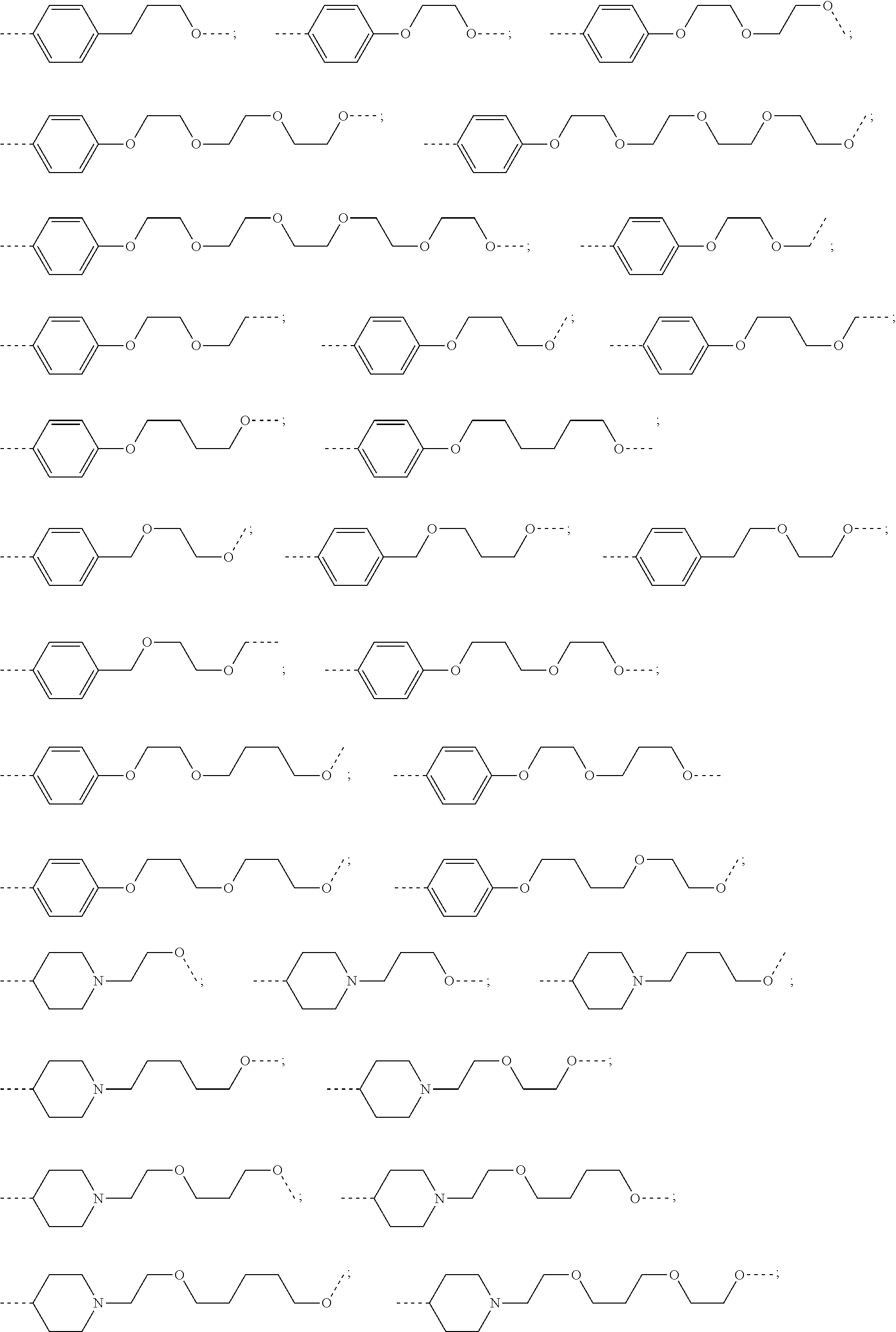

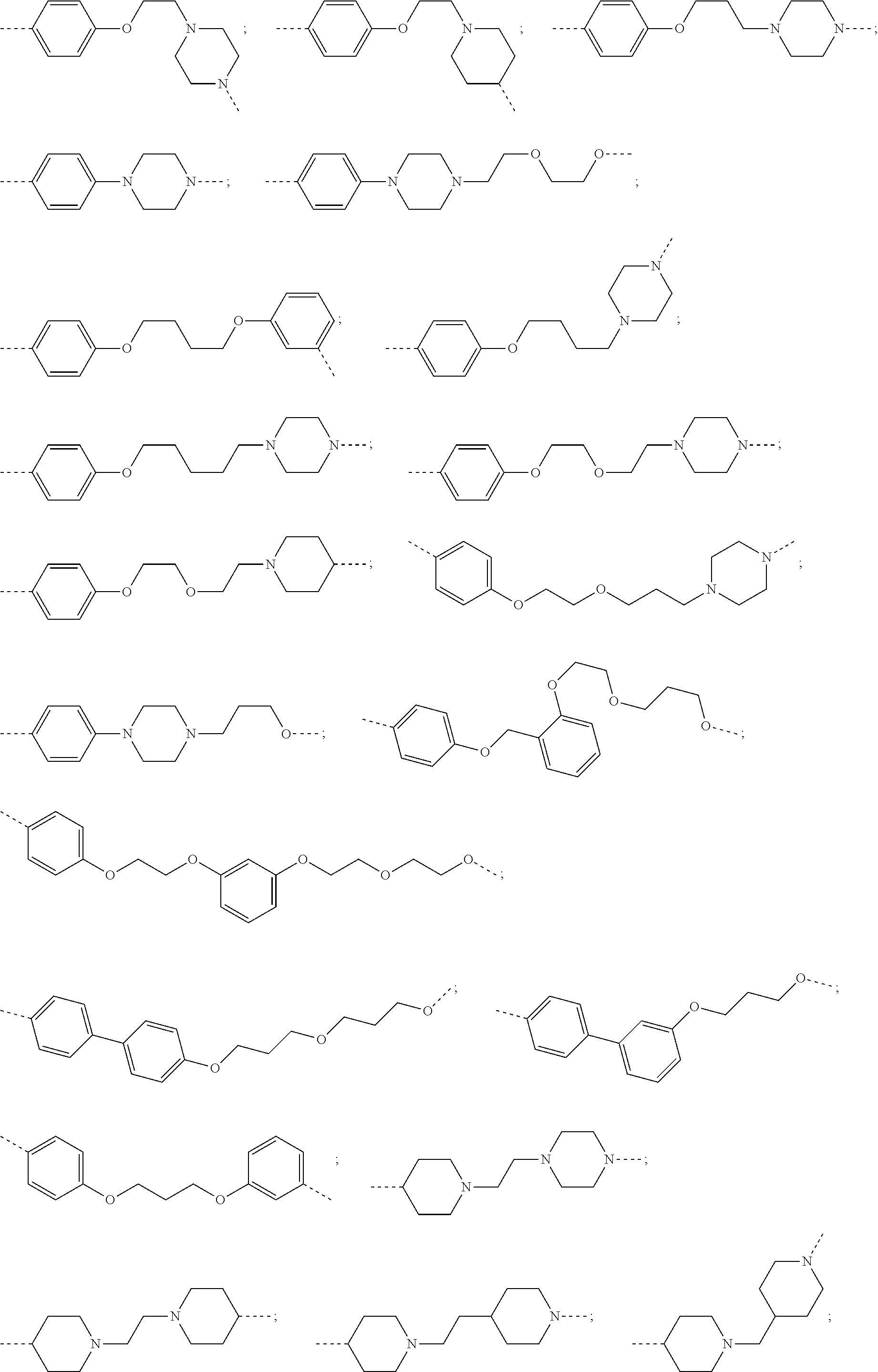

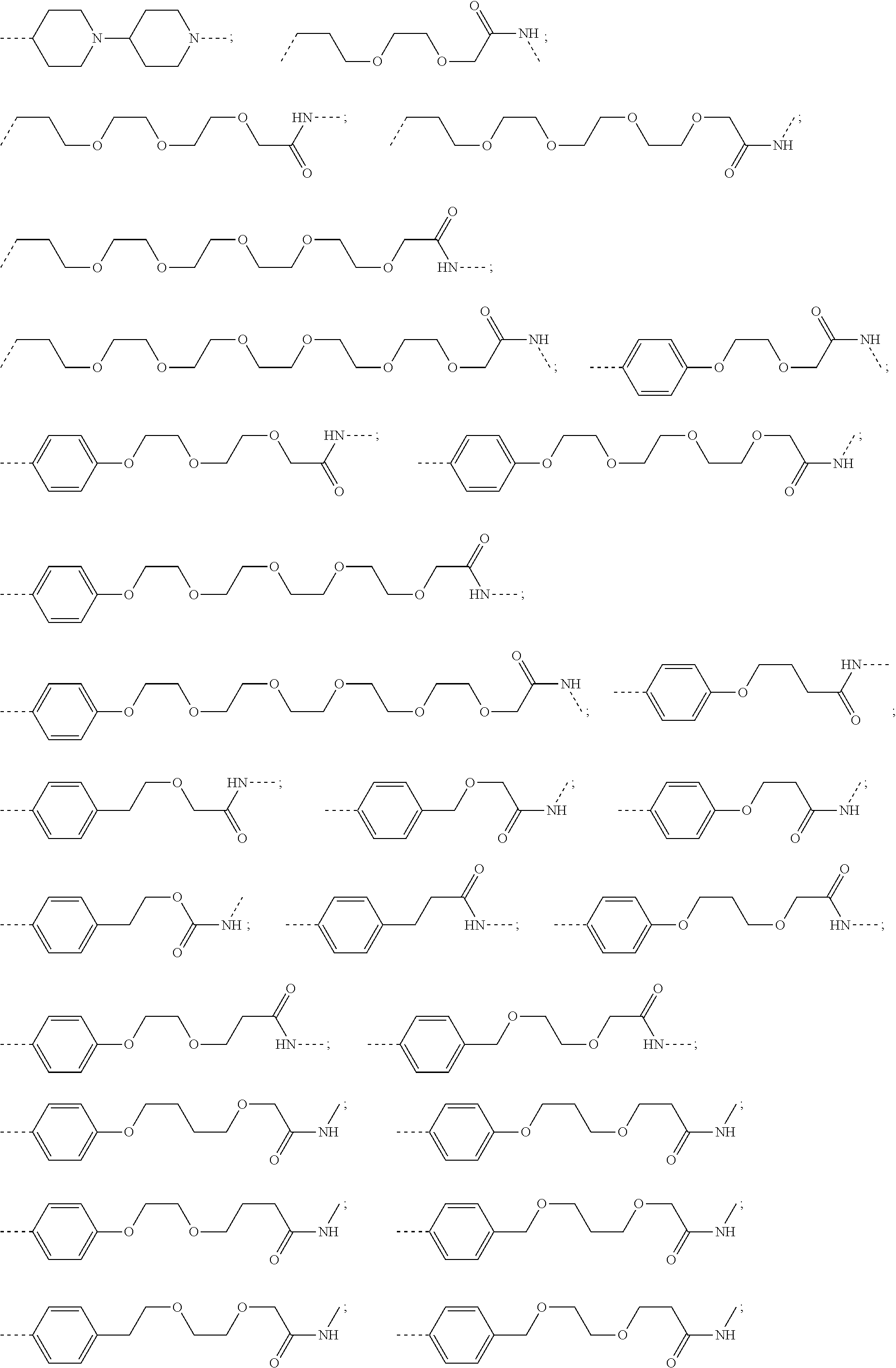

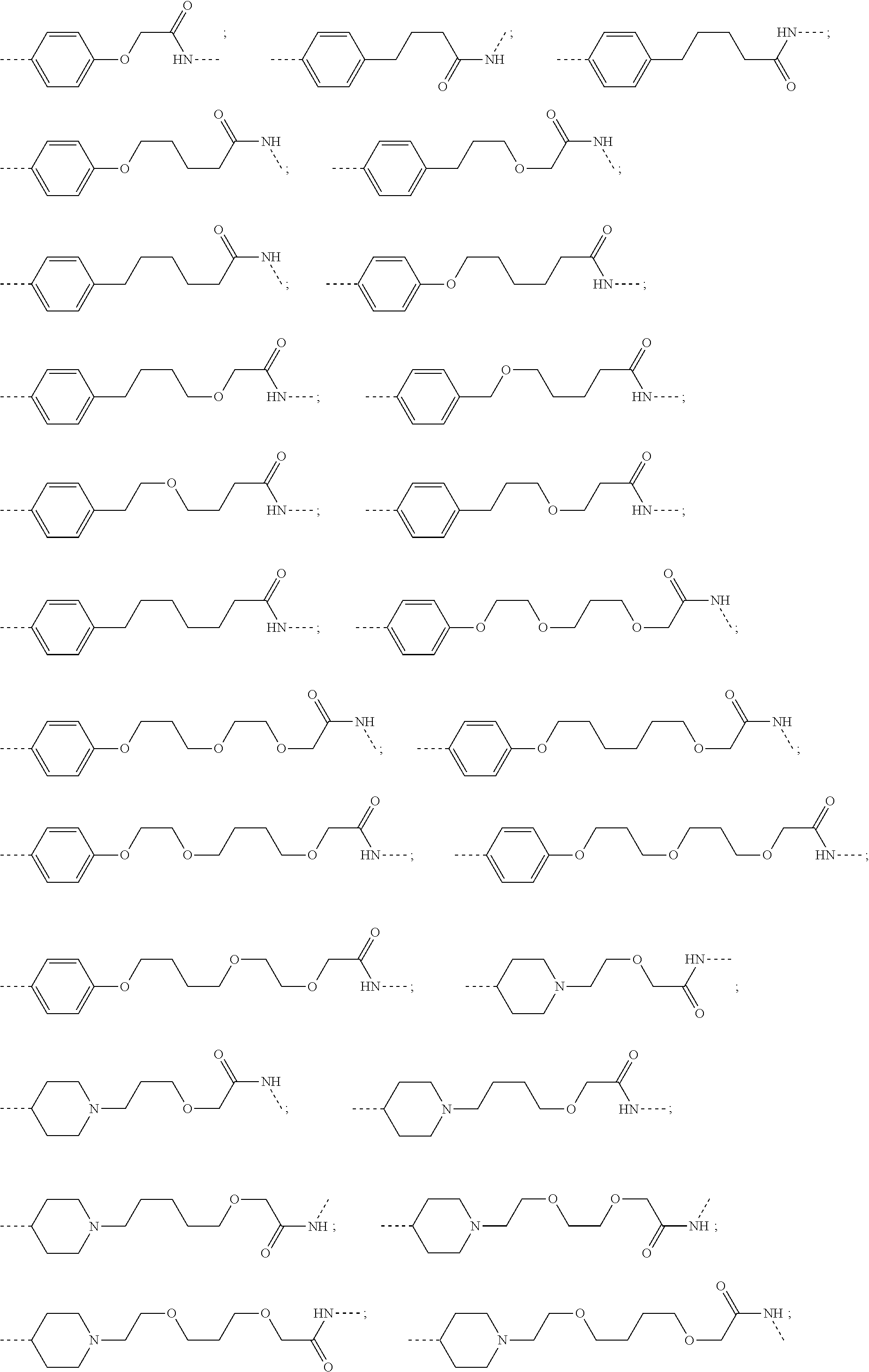

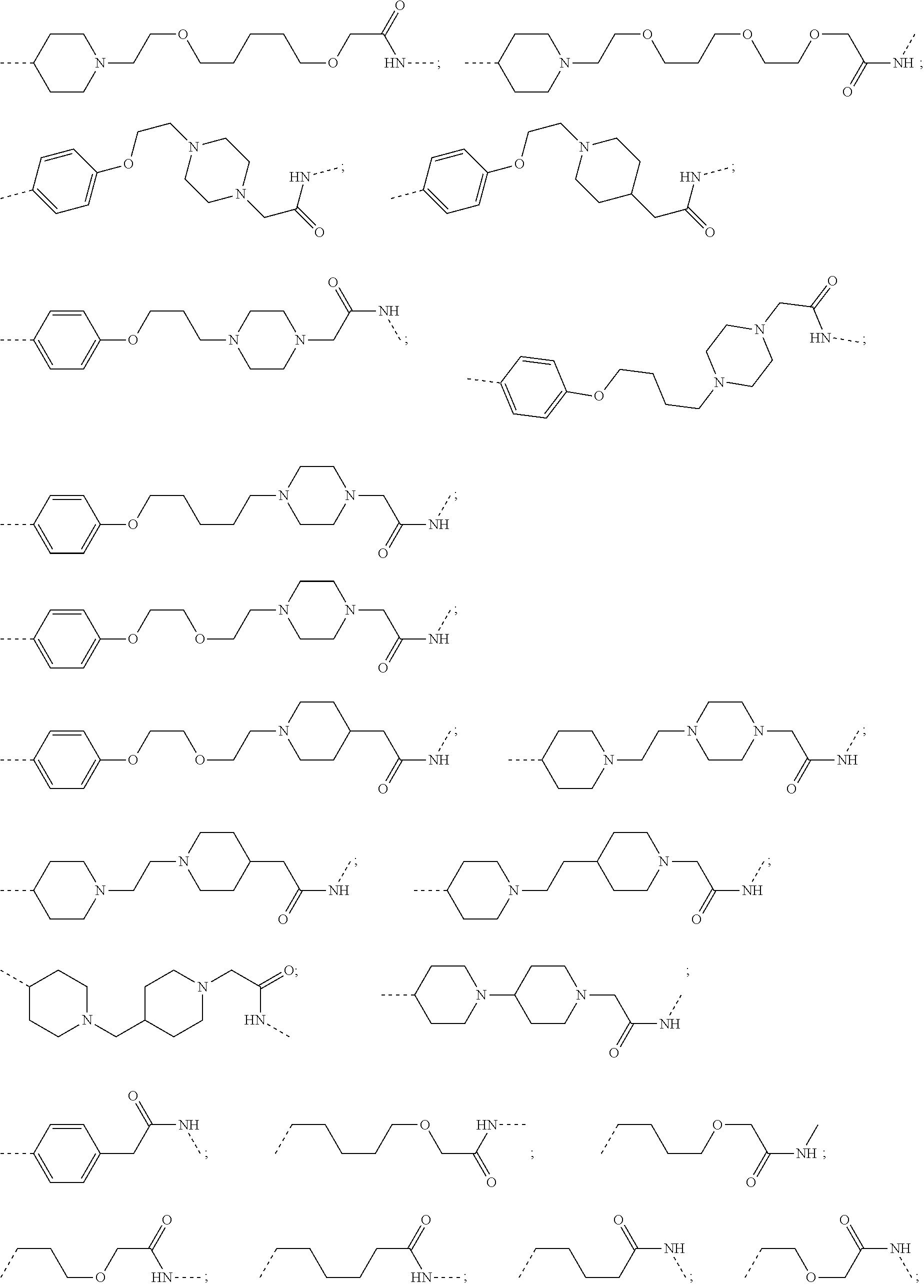

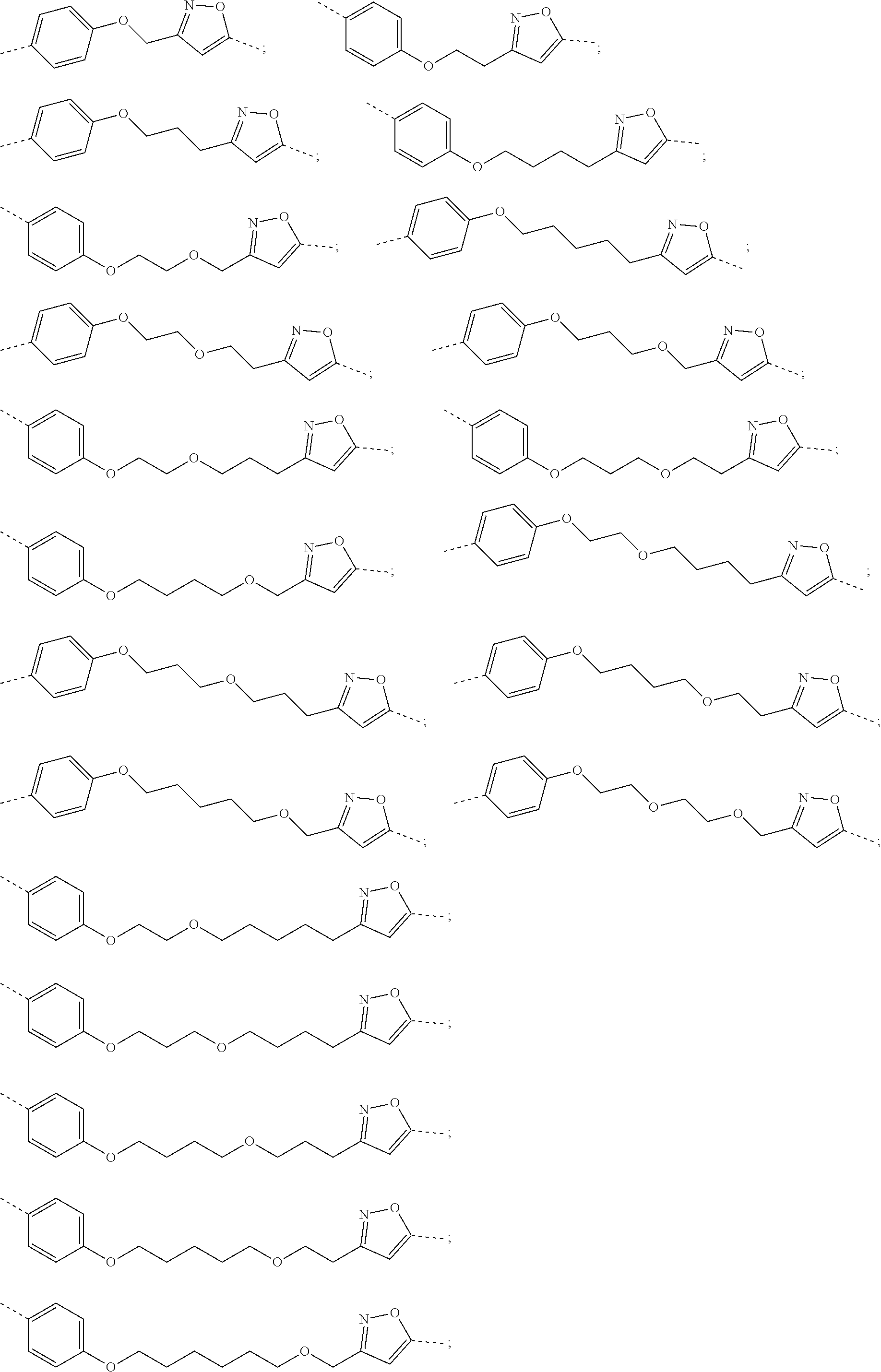

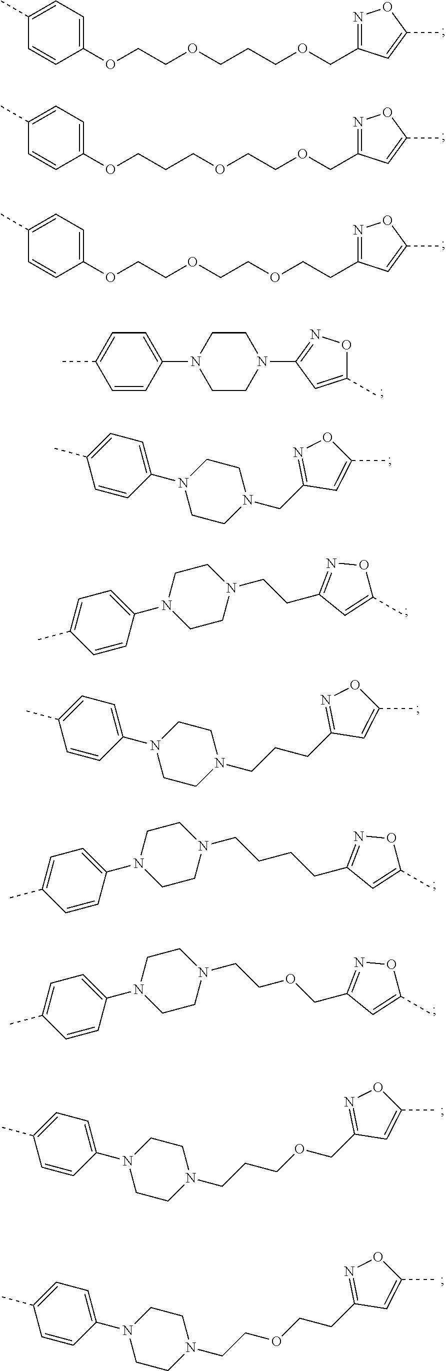

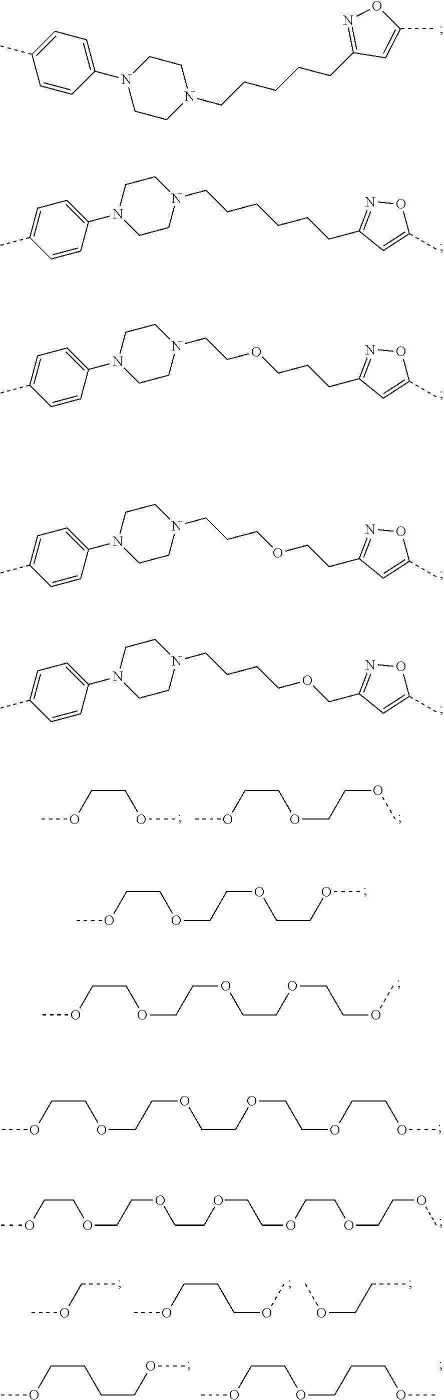

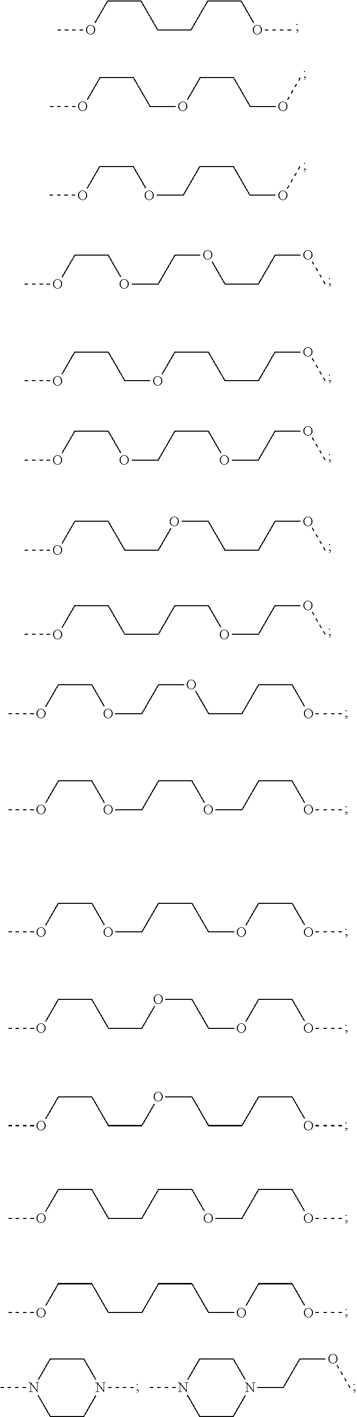

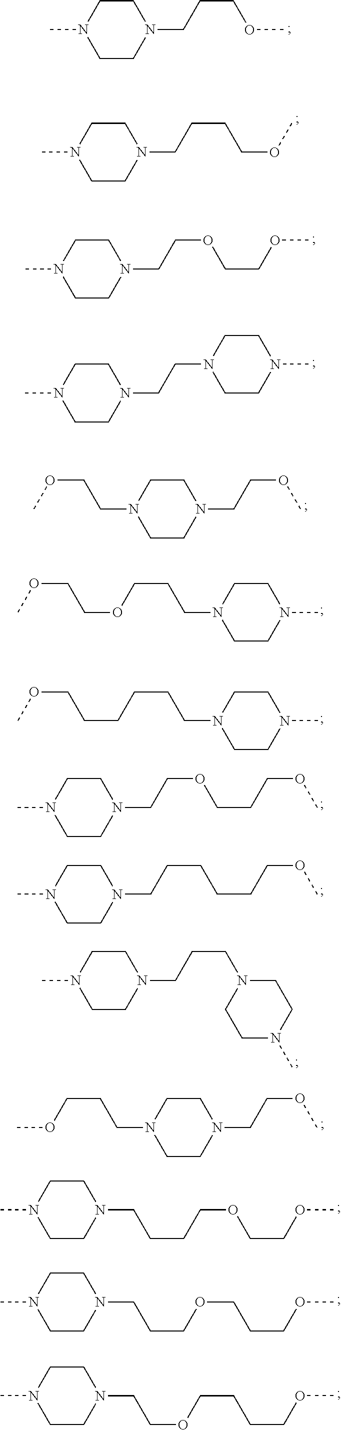

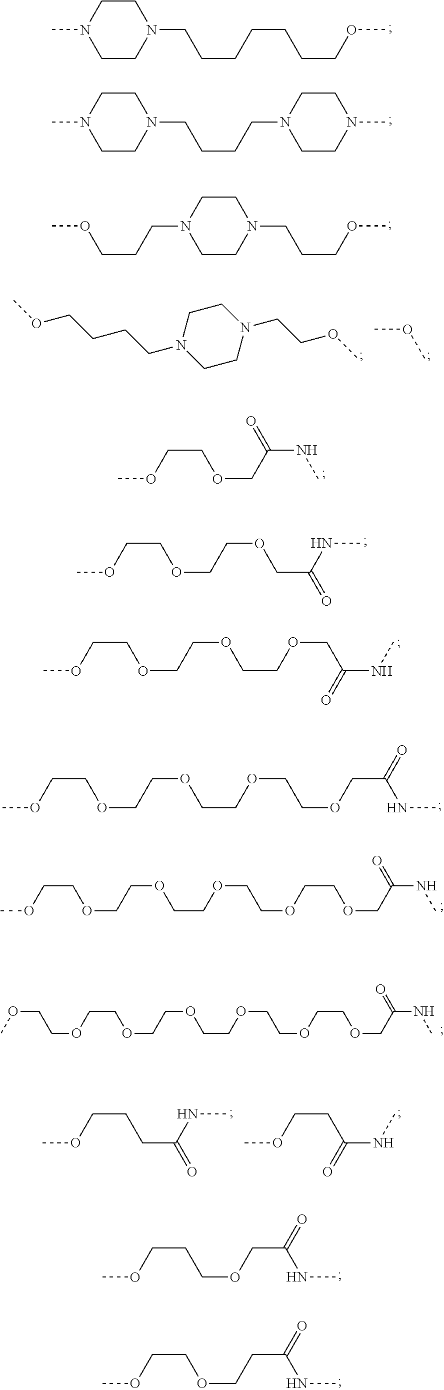

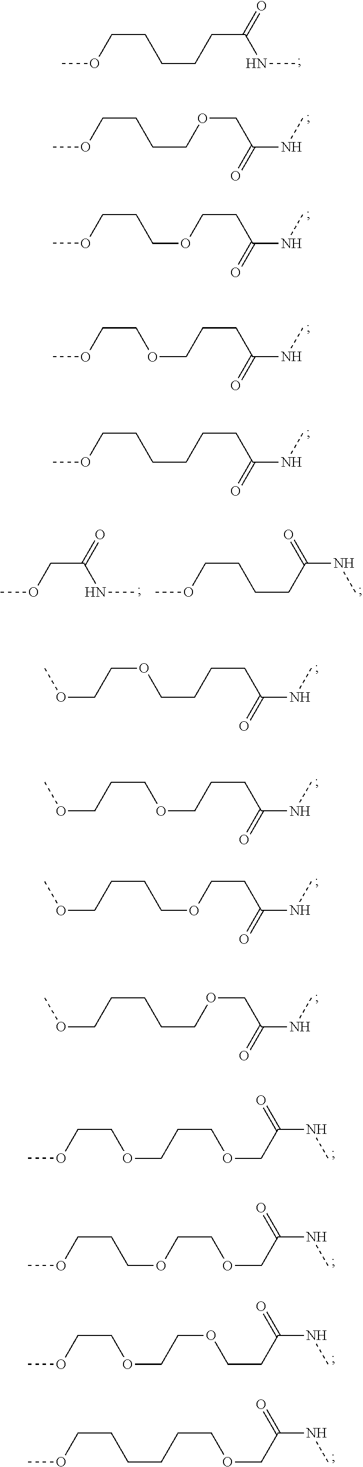

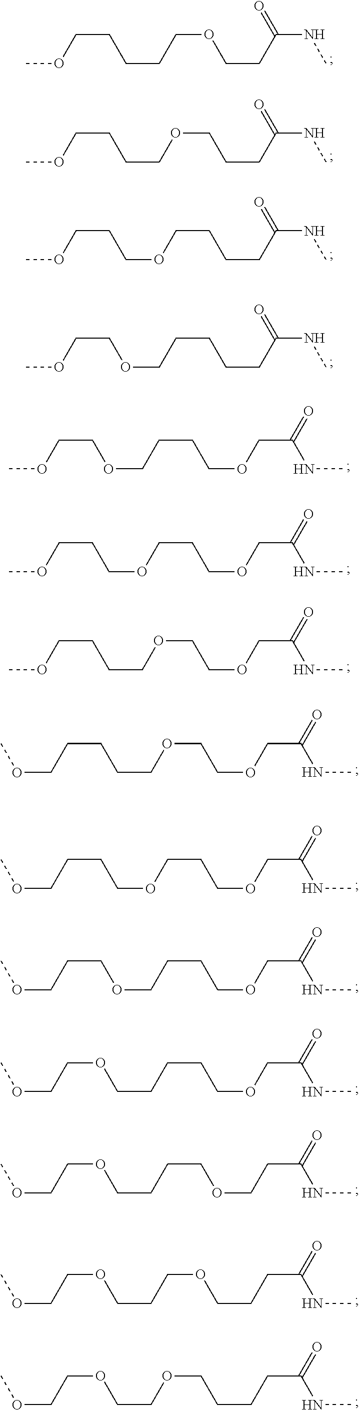

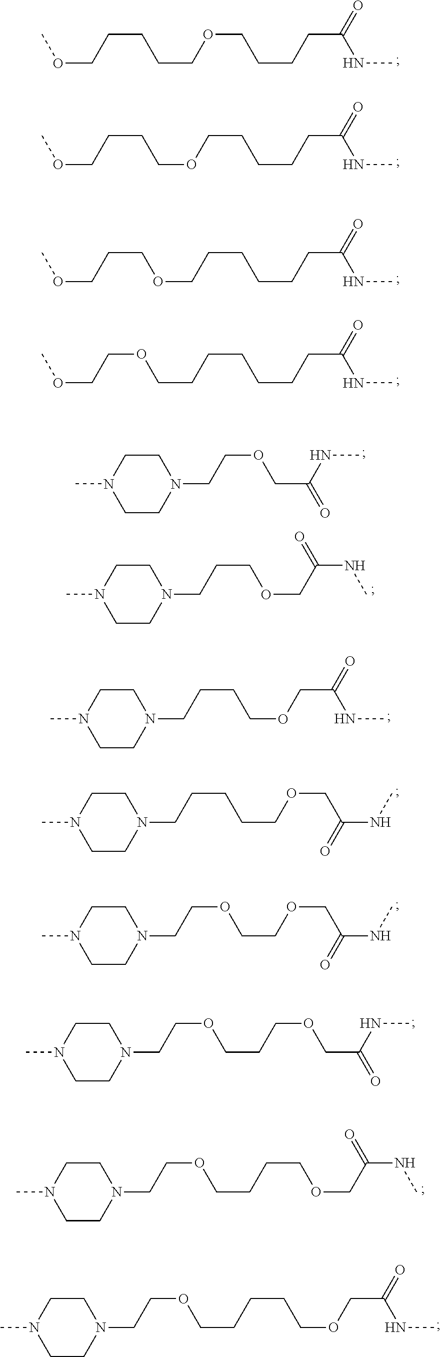

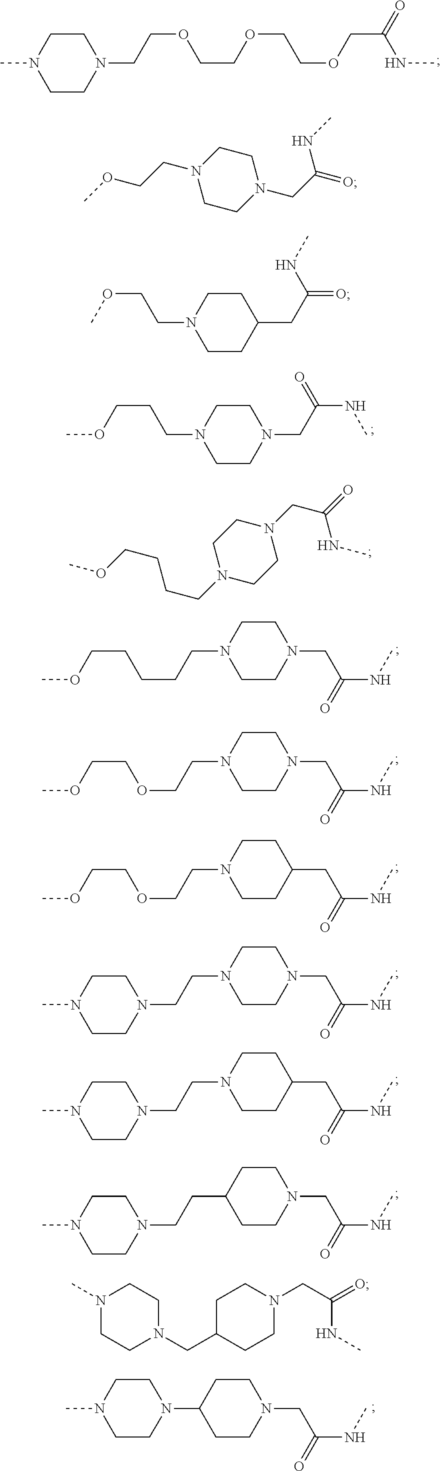

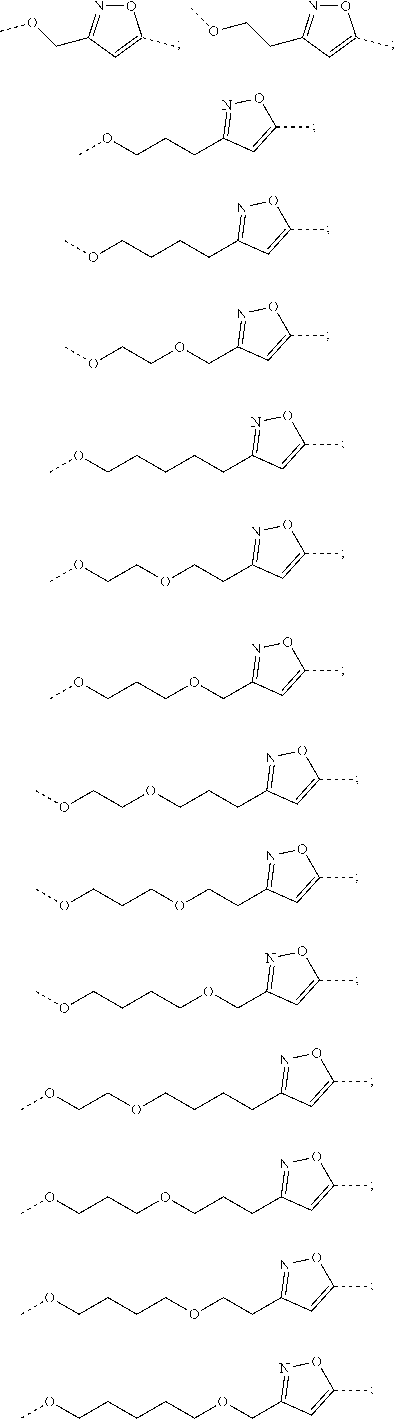

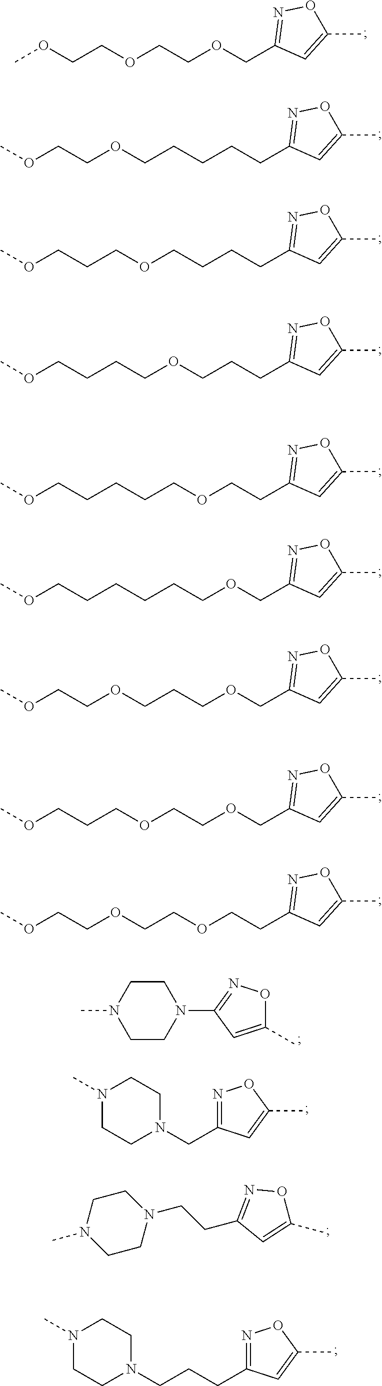

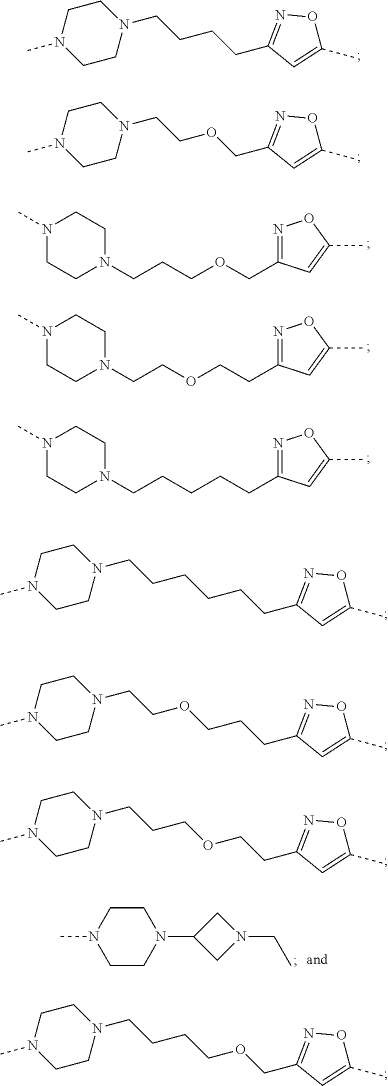

wherein a dashed bond indicates a point of attachment.

In certain embodiments, L is —C(H)(R¹⁰⁰)—, —C(R¹⁰⁰)₂—, O, —N(R¹⁰)—, —S(O)₂—, an optionally substituted C₃₋₇ cycloalkylene, an optionally substituted C₄₋₇ cycloalkenylene, or an optionally substituted 3-7 membered heterocyclylene containing 1, 2 or 3 heteroatoms selected from oxygen, nitrogen, and sulfur; wherein R¹⁰⁰ represents independently for each occurrence hydrogen, halogen, C₁₋₆ alkyl, or C₃₋₆ cycloalkyl, and R¹⁰1 is hydrogen, C₁₋₆ alkyl, or C₃₋₆ cycloalkyl. In certain embodiments, R¹⁰⁰ represents independently for each occurrence hydrogen, methyl, ethyl, isopropyl, cyclopropyl, cyclobutyl, or cyclopentyl. In certain embodiments, R¹⁰¹ is hydrogen, methyl, ethyl, isopropyl, cyclopropyl, cyclobutyl, or cyclopentyl.

In certain embodiments, L is —CH₂—Y²⁰—, —C(H)(R¹⁰⁰)—Y²⁰—, —C(R)₂—Y²⁰—, —O—Y²⁰—, —N(R¹⁰¹)—Y²⁰—, —S(O)₂—Y²⁰—, —C(O)—Y²⁰—, -(optionally substituted C₃₋₇ cycloalkylene)-Y²⁰—, -(optionally substituted C₄₋₇ cycloalkenylene)-Y²⁰—, -(optionally substituted 3-7 membered heterocyclylene containing 1, 2 or 3 heteroatoms selected from oxygen, nitrogen, and sulfur)-Y²⁰-, —Y²⁰—CH₂—, —Y²⁰—C(H)(R¹⁰⁰)—, —Y²⁰—C(R¹⁰⁰)₂—, —Y²⁰—O—, —Y²⁰—N(R¹⁰¹)—, —Y²⁰—S(O)₂—, —Y²⁰—C(O)—, —Y²⁰-(optionally substituted C₃₋₇ cycloalkylene)-, —Y²⁰-(optionally substituted C₄. cycloalkenylene)-, or —Y²⁰-(optionally substituted 3-7 membered heterocyclylene containing 1, 2 or 3 heteroatoms selected from oxygen, nitrogen, and sulfur)-; wherein R¹⁰⁰ represents independently for each occurrence hydrogen, halogen, C₁₋₆ alkyl, or C₃₋₆ cycloalkyl, and R¹⁰¹ is hydrogen, C₁₋₆ alkyl, or C₃₋₆ cycloalkyl. In certain embodiments, R¹⁰⁰ represents independently for each occurrence hydrogen, methyl, ethyl, isopropyl, cyclopropyl, cyclobutyl, or cyclopentyl. In certain embodiments, R¹⁰¹ is hydrogen, methyl, ethyl, isopropyl, cyclopropyl, cyclobutyl, or cyclopentyl.

In certain embodiments, L is one of the following:

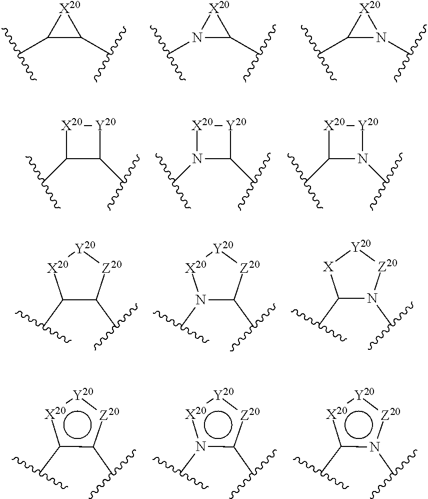

wherein X²⁰, Y²⁰, and Z²⁰ are independently —CH₂—, —C(H)(R¹⁰⁰)—, —C(R¹⁰⁰)₂—, O, —N(R¹⁰¹)—, —S(O)₂—, —C(O)—, an optionally substituted C₃₋₇ cycloalkylene, or an optionally substituted C₄₋₇ cycloalkenylene; wherein R¹⁰⁰ represents independently for each occurrence hydrogen, halogen, C₁₋₆ alkyl, or C₃₋₆ cycloalkyl, and R¹⁰1 is hydrogen, C₁₋₆ alkyl, or C₃₋₆ cycloalkyl. In certain embodiments, R¹⁰⁰ represents independently for each occurrence hydrogen, methyl, ethyl, isopropyl, cyclopropyl, cyclobutyl, or cyclopentyl. In certain embodiments, R¹⁰¹ is hydrogen, methyl, ethyl, isopropyl, cyclopropyl, cyclobutyl, or cyclopentyl.

In certain embodiments, L is one of the following:

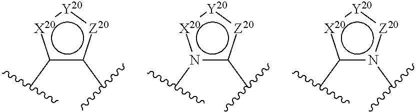

wherein X²⁰, Y²⁰, and Z²⁰ are independently —C(R¹⁰⁰)- or —N—; wherein R¹⁰⁰ represents independently for each occurrence hydrogen, halogen, C₁₋₆ alkyl, or C₃₋₆ cycloalkyl. In certain embodiments, R¹⁰⁰ represents independently for each occurrence hydrogen, methyl, ethyl, isopropyl, cyclopropyl, cyclobutyl, or cyclopentyl.

In certain embodiments, L is —X²⁰—Y²⁰—Z²⁰—, wherein X²⁰, Y²⁰, and Z²⁰ are independently —CH₂—, —C(H)(R¹⁰⁰)—, —C(R¹⁰⁰)₂—, O, —N(R¹⁰¹)—, —S(O)₂—, —C(O)—, an optionally substituted C₃₋₇ cycloalkylene, an optionally substituted C₄₋₇ cycloalkenylene, or optionally substituted 3-7 membered heterocyclylene containing 1, 2 or 3 heteroatoms selected from oxygen, nitrogen, and sulfur; wherein R¹⁰⁰ represents independently for each occurrence hydrogen, halogen, C₁₋₆ alkyl, or C₃₋₆ cycloalkyl, and R¹⁰1 is hydrogen, C₁₋₆ alkyl, or C₃₋₆ cycloalkyl. In certain embodiments, R¹⁰⁰ represents independently for each occurrence hydrogen, methyl, ethyl, isopropyl, cyclopropyl, cyclobutyl, or cyclopentyl. In certain embodiments, R¹⁰¹ is hydrogen, methyl, ethyl, isopropyl, cyclopropyl, cyclobutyl, or cyclopentyl.

In certain embodiments, L is —X²⁰=Y²⁰—Z²¹—, wherein X²⁰ and Y²⁰ are independently —C(R¹⁰⁰)- or —N—, and Z²¹ is —CH₂—, —C(H)(R¹⁰⁰)—, —C(R¹⁰¹)₂—, O, —N(R¹⁰¹)—, —S(O)₂—, —C(O)—, an optionally substituted C₃₋₇ cycloalkylene, or an optionally substituted C₄₋₇ cycloalkenylene; wherein R¹⁰⁰ represents independently for each occurrence hydrogen, halogen, C₁₋₆ alkyl, or C₃₋₆ cycloalkyl, and R¹⁰¹ is hydrogen, C₁₋₆ alkyl, or C₃₋₆ cycloalkyl. In certain embodiments, R¹⁰⁰ represents independently for each occurrence hydrogen, methyl, ethyl, isopropyl, cyclopropyl, cyclobutyl, or cyclopentyl. In certain embodiments, R¹⁰¹ is hydrogen, methyl, ethyl, isopropyl, cyclopropyl, cyclobutyl, or cyclopentyl.

In certain embodiments, L is —C≡C—Z²⁰—, wherein Z²⁰ is —CH₂—, —C(H)(R¹⁰⁰)—, —C(R¹⁰⁰)₂—O, —N(R¹⁰¹)—, —S(O)₂—, —C(O)—, an optionally substituted C₃₋₇ cycloalkylene, or an optionally substituted C₄₋₇ cycloalkenylene; wherein R¹⁰⁰ represents independently for each occurrence hydrogen, halogen, C₁₋₆ alkyl, or C₃₋₆ cycloalkyl, and R¹⁰1 is hydrogen, C₁₋₆ alkyl, or C₃₋₆ cycloalkyl. In certain embodiments, R¹⁰⁰ represents independently for each occurrence hydrogen, methyl, ethyl, isopropyl, cyclopropyl, cyclobutyl, or cyclopentyl. In certain embodiments, R⁰¹ is hydrogen, methyl, ethyl, isopropyl, cyclopropyl, cyclobutyl, or cyclopentyl.

In certain embodiments, L is one of the following:

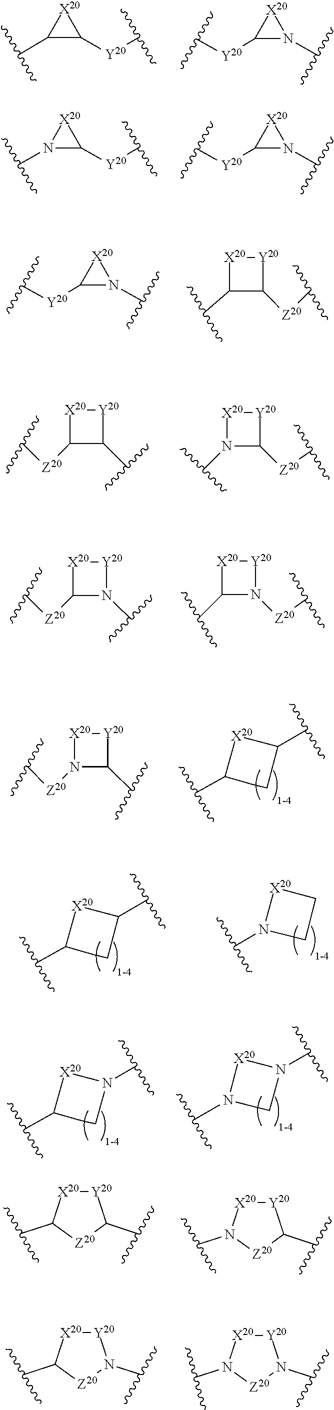

wherein X²⁰, Y²⁰, and Z²⁰ are independently —CH₂—, —C(H)(R¹⁰⁰)—, —C(R¹⁰⁰)₂—, O, —N(R¹⁰¹)—, —S(O)₂—, —C(O)—, an optionally substituted C₃₋₇ cycloalkylene, an optionally substituted C₄₋₇ cycloalkenylene, or optionally substituted 3-7 membered heterocyclylene containing 1, 2 or 3 heteroatoms selected from oxygen, nitrogen, and sulfur; wherein R¹⁰⁰ represents independently for each occurrence hydrogen, halogen, C₁₋₆ alkyl, or C₃₋₆ cycloalkyl, and R¹⁰¹ is hydrogen, C₁₋₆ alkyl, or C₃₋₆ cycloalkyl. In certain embodiments, R¹⁰⁰ represents independently for each occurrence hydrogen, methyl, ethyl, isopropyl, cyclopropyl, cyclobutyl, or cyclopentyl. In certain embodiments, R¹⁰¹ is hydrogen, methyl, ethyl, isopropyl, cyclopropyl, cyclobutyl, or cyclopentyl.

In certain embodiments, L is one of the following:

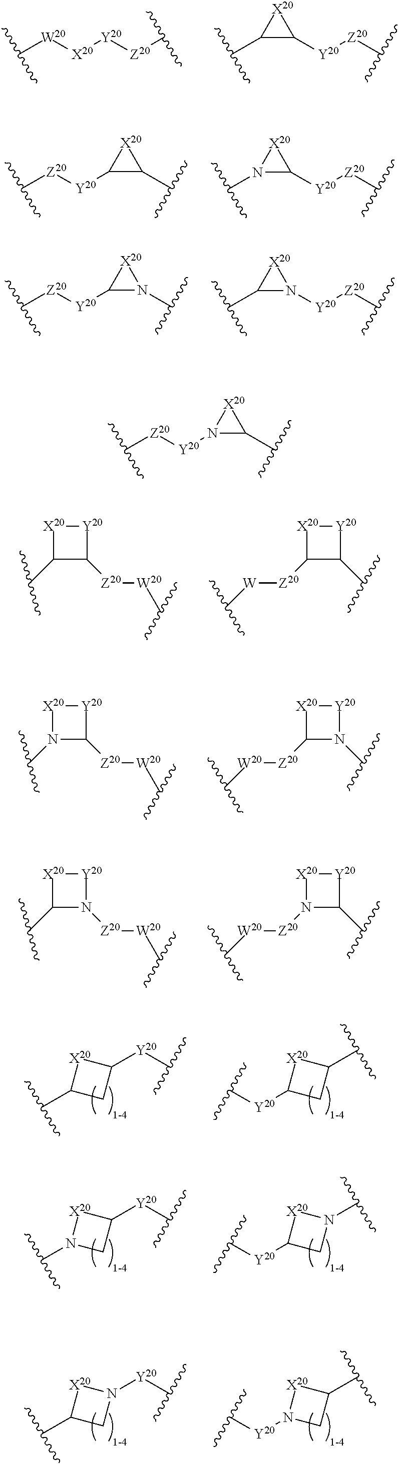

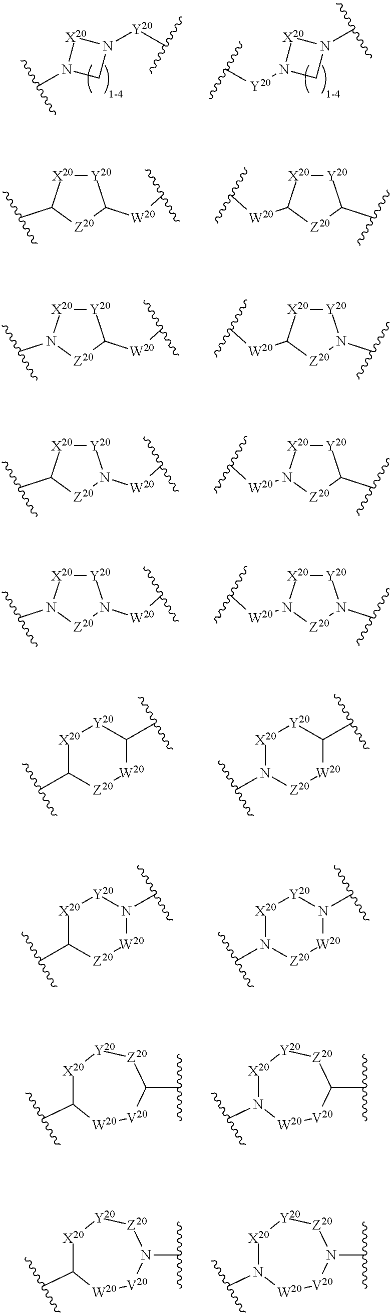

wherein V²⁰, W²⁰, X²⁰, Y²⁰, and Z²⁰ are independently —CH₂—, —C(H)(R¹⁰°)-, —C(R¹⁰⁰)₂₋₁₀, —N(R¹⁰¹)—, —S(O)₂—, —C(O)—, an optionally substituted C₃₋₇ cycloalkylene, an optionally substituted C₄₋₇ cycloalkenylene, or optionally substituted 3-7 membered heterocyclylene containing 1, 2 or 3 heteroatoms selected from oxygen, nitrogen, and sulfur; wherein R¹⁰⁰ represents independently for each occurrence hydrogen, halogen, C₁₋₆alkyl, or C₃₋₆ cycloalkyl, and Riot is hydrogen, C₁₋₆alkyl, or C₃₋₆ cycloalkyl. In certain embodiments, R¹⁰⁰ represents independently for each occurrence hydrogen, methyl, ethyl, isopropyl, cyclopropyl, cyclobutyl, or cyclopentyl. In certain embodiments, R¹ is hydrogen, methyl, ethyl, isopropyl, cyclopropyl, cyclobutyl, or cyclopentyl.

In certain embodiments, L is one of the following:

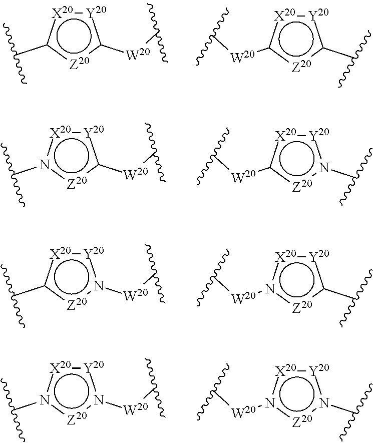

wherein W²⁰, X²⁰, Y²⁰, and Z²⁰ are independently —C(R¹⁰⁰)— or —N—; wherein R¹⁰⁰ represents independently for each occurrence hydrogen, halogen, C₁₋₆ alkyl, or C₃₋₆ cycloalkyl. In certain embodiments, R¹⁰⁰ represents independently for each occurrence hydrogen, methyl, ethyl, isopropyl, cyclopropyl, cyclobutyl, or cyclopentyl.

In certain embodiments, L is one of the following:

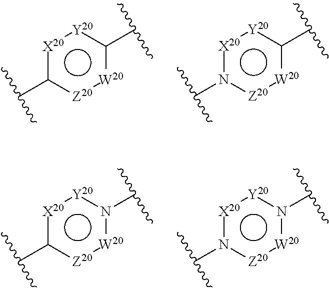

wherein W²⁰, X²⁰, Y²⁰, and Z²⁰ are independently —C(R¹⁰⁰)— or —N—; wherein R¹⁰⁰ represents independently for each occurrence hydrogen, halogen, C₁₋₆ alkyl, or C₃₋₆ cycloalkyl. In certain embodiments, R¹⁰⁰ represents independently for each occurrence hydrogen, methyl, ethyl, isopropyl, cyclopropyl, cyclobutyl, or cyclopentyl.

In certain embodiments, L is one of the following:

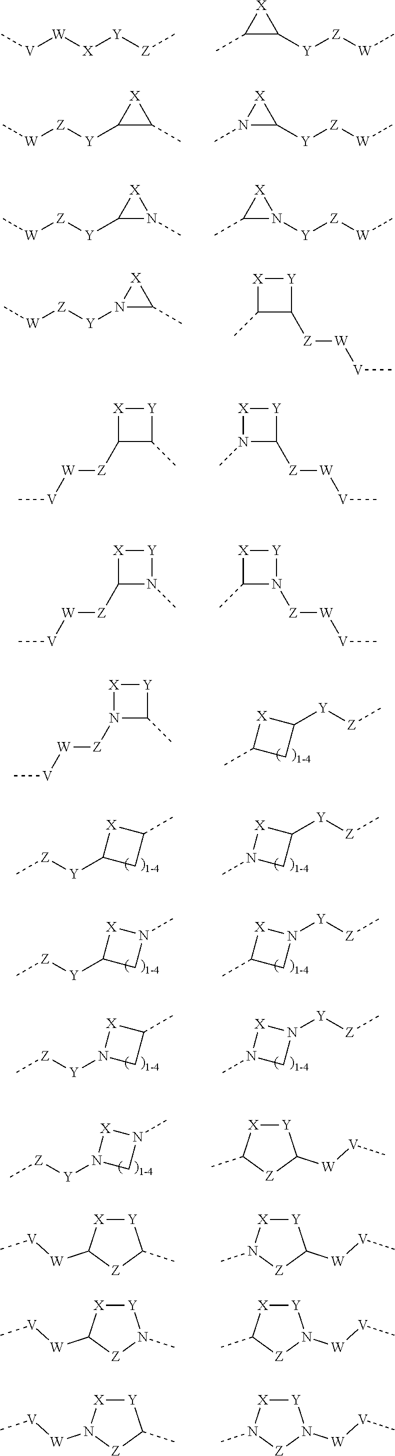

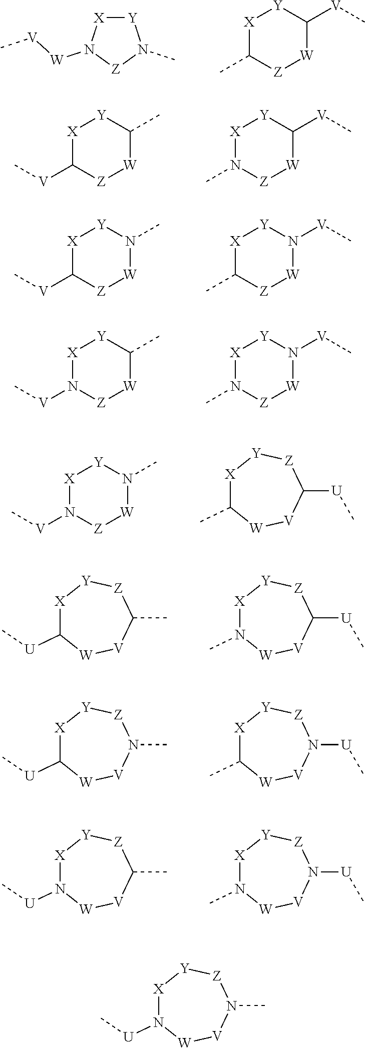

wherein U, V, W, X, Y, and Z are independently —CH₂—, —C(H)(R Z⁰)——C(R¹⁰⁰)₂—, O, —N(R¹⁰¹)—, —S(O)₂—, —C(O)—, an optionally substituted C₃₋₇ cycloalkylene, an optionally substituted C₄₋₇cycloalkenylene, or optionally substituted 3-7 membered heterocyclylene containing 1, 2 or 3 heteroatoms selected from oxygen, nitrogen, and sulfur; wherein R¹⁰⁰ represents independently for each occurrence hydrogen, halogen, C₁₋₆alkyl, or C₃₋₆ cycloalkyl; and R¹⁰¹ is hydrogen, C₁₋₆ alkyl, or C₃₋₆ cycloalkyl; and a dashed bond indicates a point of attachment. In certain embodiments, R¹⁰⁰ represents independently for each occurrence hydrogen, methyl, ethyl, isopropyl, cyclopropyl, cyclobutyl, or cyclopentyl. In certain embodiments, R¹⁰¹ is hydrogen, methyl, ethyl, isopropyl, cyclopropyl, cyclobutyl, or cyclopentyl.

In certain embodiments, L is one of the following:

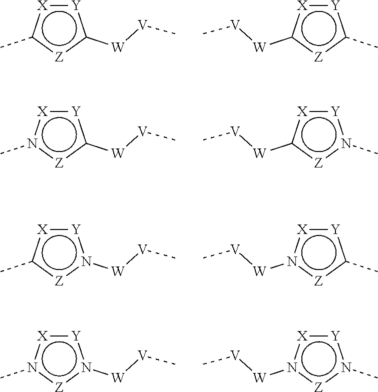

wherein X, Y, and Z are independently —C(R¹⁰⁰)— or —N—; V and W are independently —CH₂—, —C(H)(R¹⁰¹)—, —C(R¹⁰⁰)₂—, O, —N(R¹⁰¹)—, —S(O)₂—, —C(O)—, an optionally substituted C₃₋₇ cycloalkylene, an optionally substituted C₄₋₇ cycloalkenylene, or optionally substituted 3-7 membered heterocyclylene containing 1, 2 or 3 heteroatoms selected from oxygen, nitrogen, and sulfur; wherein R¹⁰⁰ represents independently for each occurrence hydrogen, halogen, C₁₋₆ alkyl, or C₃₋₆ cycloalkyl; R¹⁰¹ is hydrogen, C₁₋₆ alkyl, or C₃₋₆ cycloalkyl; and a dashed bond indicates a point of attachment. In certain embodiments, R¹⁰⁰ represents independently for each occurrence hydrogen, methyl, ethyl, isopropyl, cyclopropyl, cyclobutyl, or cyclopentyl. In certain embodiments, R⁰¹ is hydrogen, methyl, ethyl, isopropyl, cyclopropyl, cyclobutyl, or cyclopentyl.

In certain embodiments, L is one of the following:

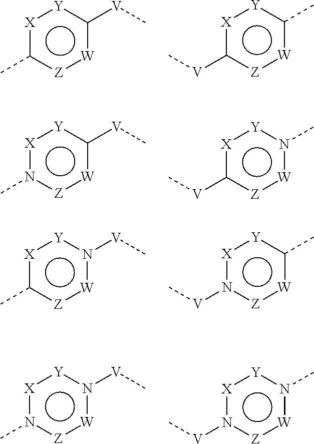

—C(R¹⁰⁰)₂—, O, —N(R¹⁰¹)—, —S(O)₂—, —C(O)—, an optionally substituted C₃₋₇ cycloalkylene, an optionally substituted C₄₋₇ cycloalkenylene, or optionally substituted 3-7 membered heterocyclylene containing 1, 2 or 3 heteroatoms selected from oxygen, nitrogen, and sulfur; wherein R¹⁰⁰ represents independently for each occurrence hydrogen, halogen, C₁₋₆ alkyl, or C₃₋₆ cycloalkyl; R¹⁰¹ is hydrogen, C₁₋₆ alkyl, or C₃₋₆ cycloalkyl; and a dashed bond indicates a point of attachment. In certain embodiments, R¹⁰⁰ represents independently for each occurrence hydrogen, methyl, ethyl, isopropyl, cyclopropyl, cyclobutyl, or cyclopentyl. In certain embodiments, R¹⁰¹ is hydrogen, methyl, ethyl, isopropyl, cyclopropyl, cyclobutyl, or cyclopentyl.

In certain embodiments, L is one of the following:

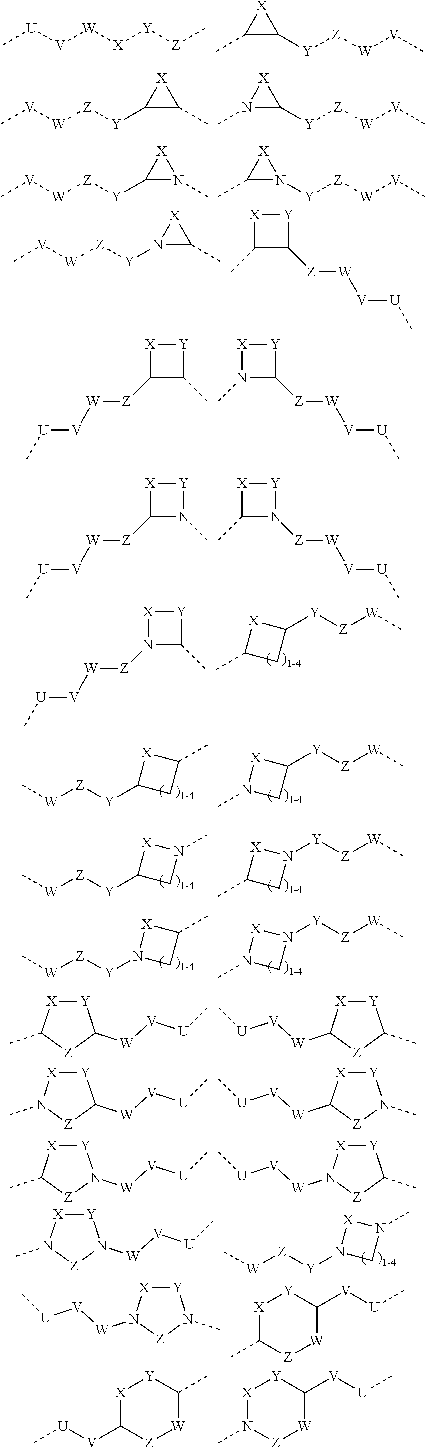

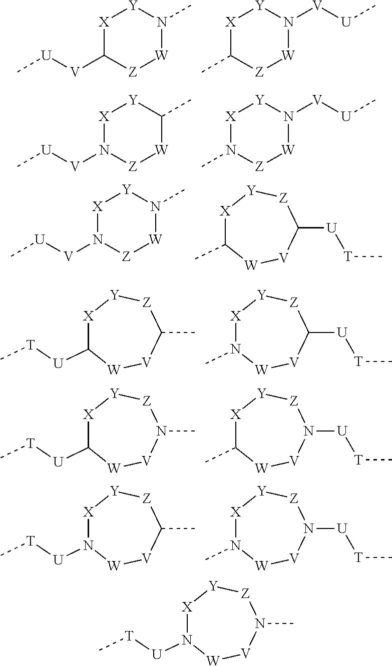

wherein T, U, V, W, X, Y, and Z are independently —CH₂—, —C(H)(R¹⁰⁰)—, —C(R¹⁰⁰)₂—, O, —N(R¹⁰¹)—, —S(O)₂—, —C(O)—, an optionally substituted C₃₋₇ cycloalkylene, an optionally substituted C₄₋₇ cycloalkenylene, or optionally substituted 3-7 membered heterocyclylene containing 1, 2 or 3 heteroatoms selected from oxygen, nitrogen, and sulfur; wherein R¹⁰⁰ represents independently for each occurrence hydrogen, halogen, C₁₋₆ alkyl, or C₃₋₆ cycloalkyl; R¹⁰¹ is hydrogen, C₁₋₆ alkyl, or C₃₋₆ cycloalkyl; and a dashed bond indicates a point of attachment. In certain embodiments, R¹⁰⁰ represents independently for each occurrence hydrogen, methyl, ethyl, isopropyl, cyclopropyl, cyclobutyl, or cyclopentyl. In certain embodiments, R¹⁰¹ is hydrogen, methyl, ethyl, isopropyl, cyclopropyl, cyclobutyl, or cyclopentyl.

In certain embodiments, L is one of the following:

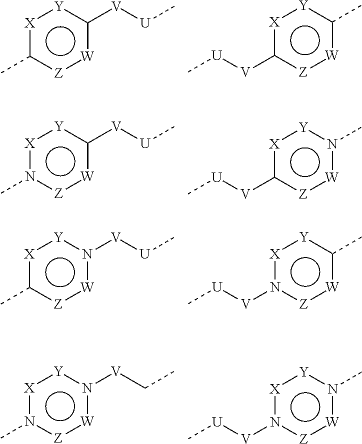

wherein W, X, Y, and Z are independently —C(R¹⁰⁰)— or —N—; U and V are independently —CH₂—, —C(H)(R¹⁰⁰)—, —C(R¹⁰⁰)₂—, O, —N(R¹⁰¹)—, —S(O)₂—, —C(O)—, an optionally substituted C₃₋₇ cycloalkylene, an optionally substituted C₄₋₇ cycloalkenylene, or optionally substituted 3-7 membered heterocyclylene containing 1, 2 or 3 heteroatoms selected from oxygen, nitrogen, and sulfur; R⁰⁰ represents independently for each occurrence hydrogen, halogen, C₁₋₆ alkyl, or C₃₋₆ cycloalkyl; R⁰¹° is hydrogen, C₁₋₆ alkyl, or C₃₋₆ cycloalkyl; and a dashed bond indicates a point of attachment. In certain embodiments, R¹⁰⁰ represents independently for each occurrence hydrogen, methyl, ethyl, isopropyl, cyclopropyl, cyclobutyl, or cyclopentyl. In certain embodiments, R¹⁰¹ is hydrogen, methyl, ethyl, isopropyl, cyclopropyl, cyclobutyl, or cyclopentyl.

In certain embodiments, L is one of the following:

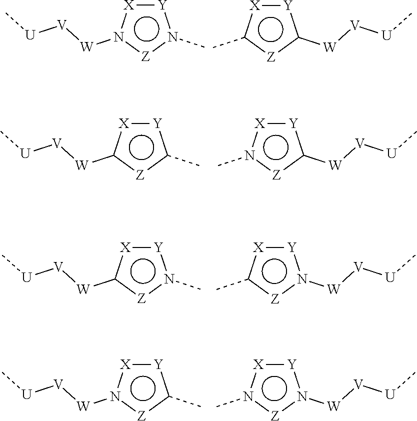

wherein X, Y, and Z are independently —C(R¹⁰⁰)- or —N—; U, V, and W are independently —CH₂—, —C(H)(R¹⁰⁰)—, —C(R¹⁰⁰)₂—, O, —N(R¹⁰¹)—, —S(O)₂—, —C(O)—, an optionally substituted C₃₋₇ cycloalkylene, an optionally substituted C₄₋₇ cycloalkenylene, or optionally substituted 3-7 membered heterocyclylene containing 1, 2 or 3 heteroatoms selected from oxygen, nitrogen, and sulfur; wherein R¹⁰⁰ represents independently for each occurrence hydrogen, halogen, C₁₋₆ alkyl, or C₃₋₆ cycloalkyl; R¹⁰¹ is hydrogen, C₁₋₆ alkyl, or C₃₋₆ cycloalkyl; and a dashed bond indicates a point of attachment. In certain embodiments, R¹⁰⁰ represents independently for each occurrence hydrogen, methyl, ethyl, isopropyl, cyclopropyl, cyclobutyl, or cyclopentyl. In certain embodiments, R¹⁰¹ is hydrogen, methyl, ethyl, isopropyl, cyclopropyl, cyclobutyl, or cyclopentyl.

In certain embodiments, L is one of the following:

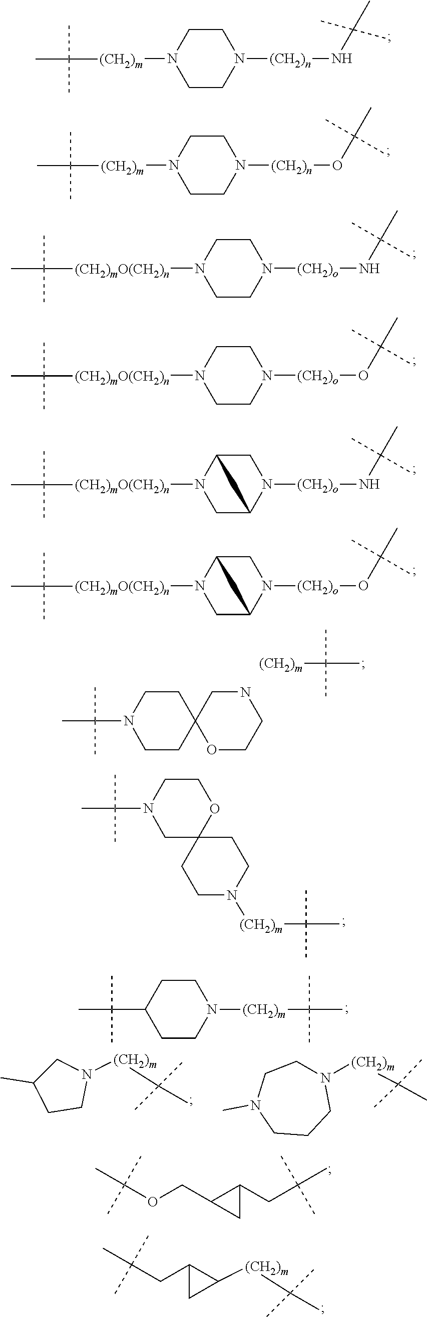

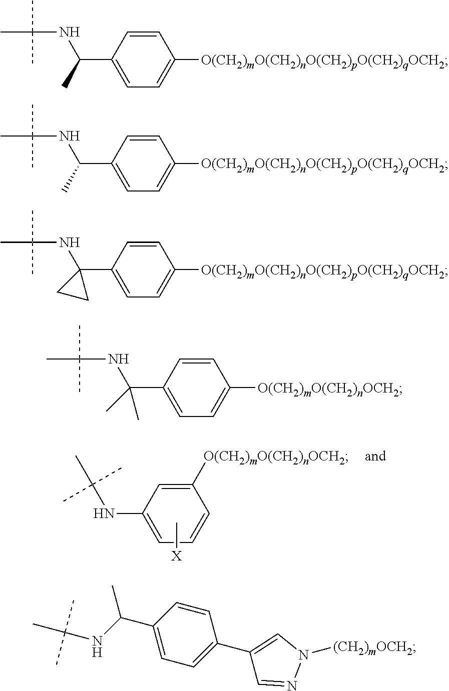

wherein
variables m, n, o, p, and q are independently 0, 1, 2, 3, 4, 5, 6, 7, 8, 9, 10, 11, 12, 13, 14, 15, 16, 17, 18, 19, or 20.

In certain embodiments, L is one of the following:

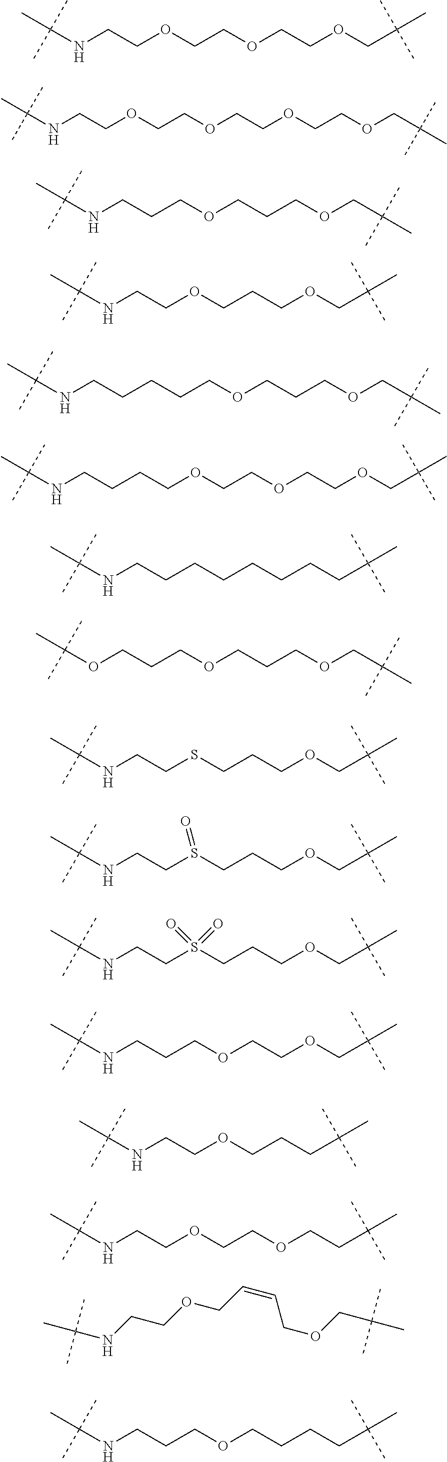

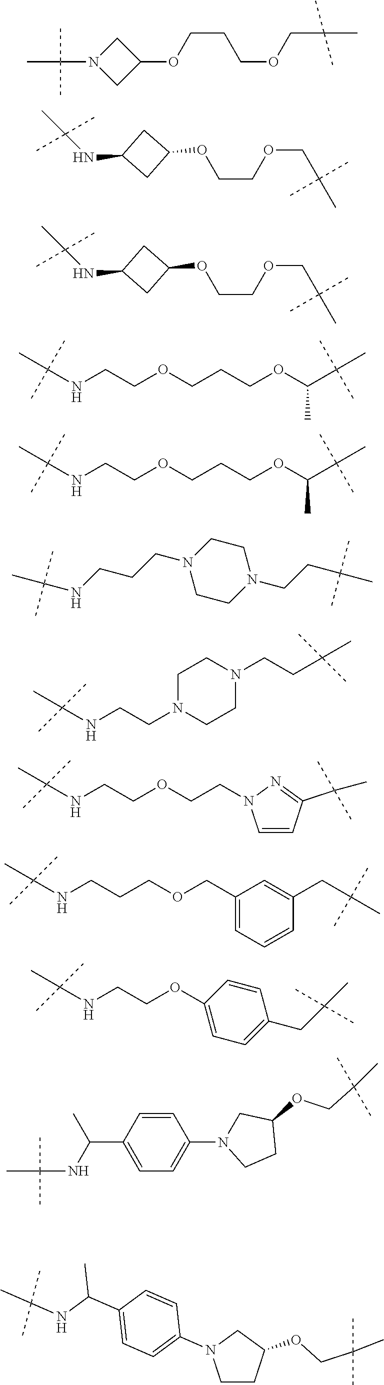

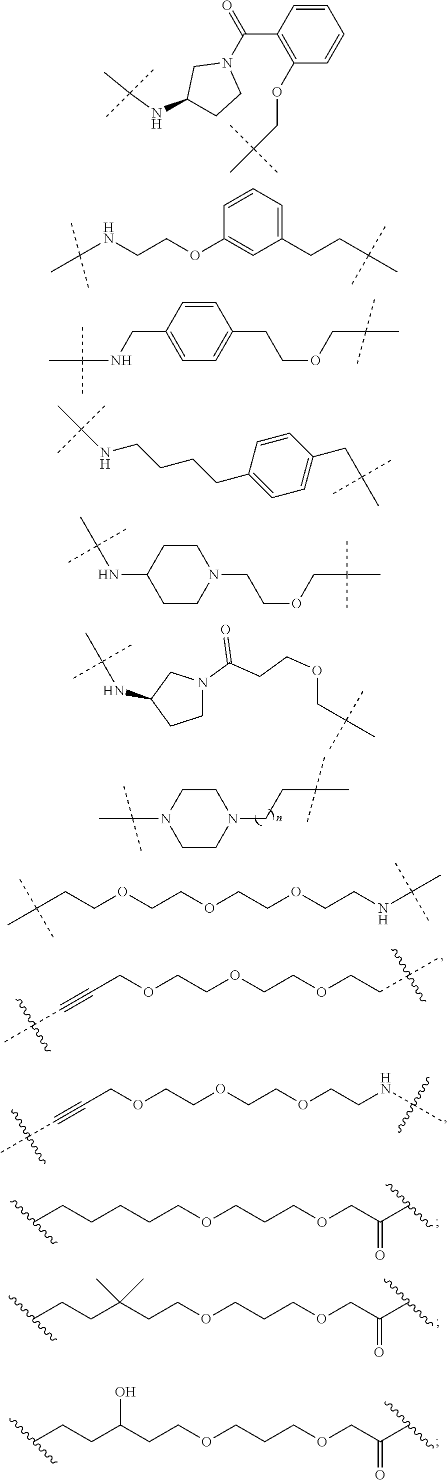

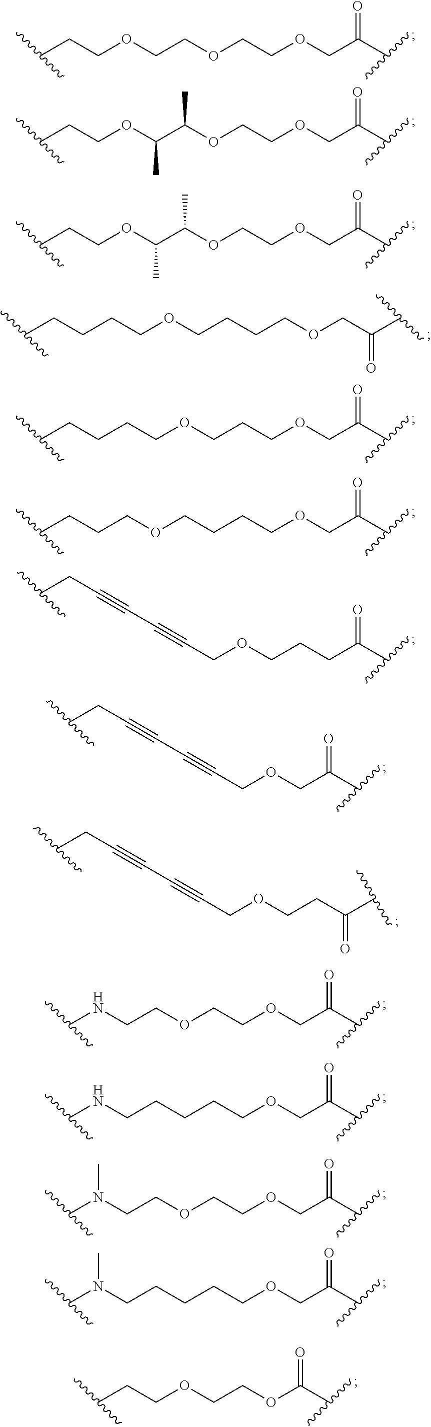

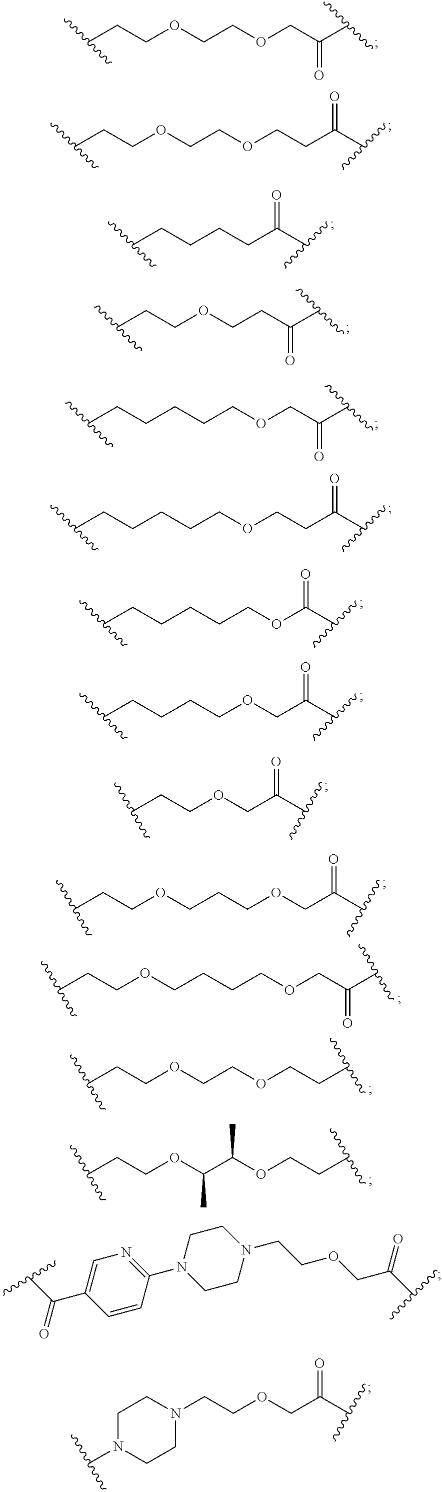

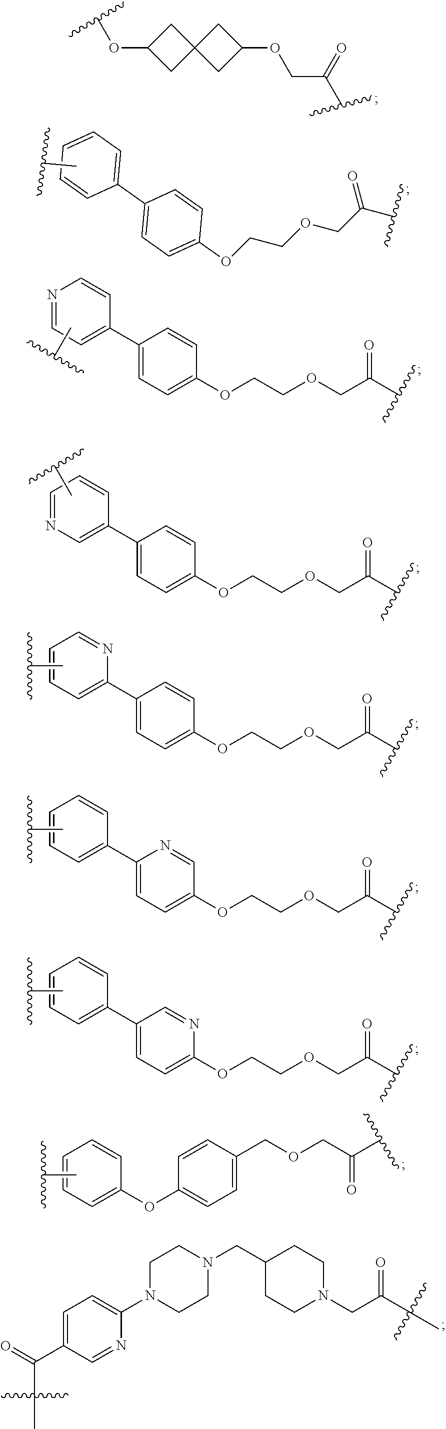

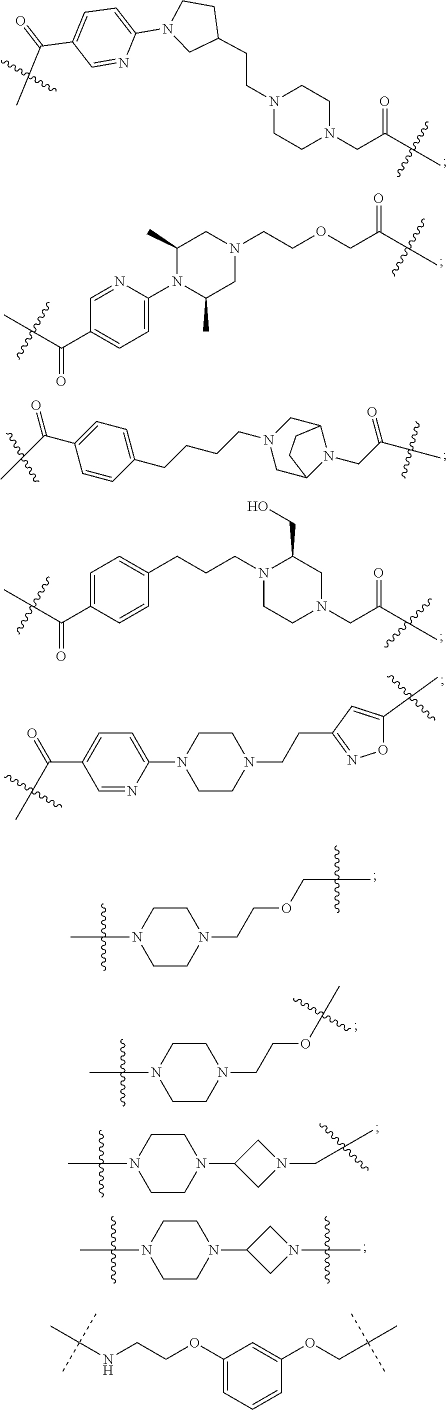

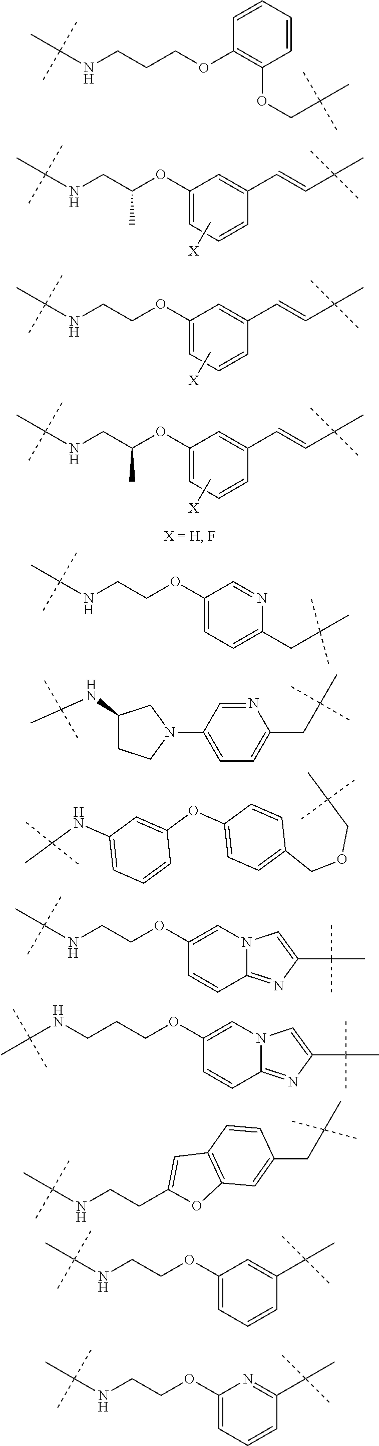

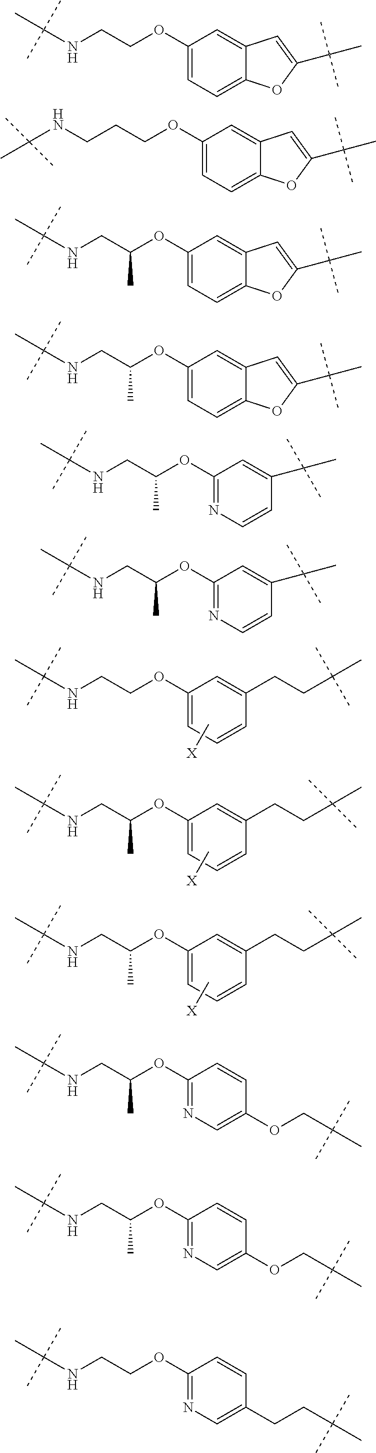

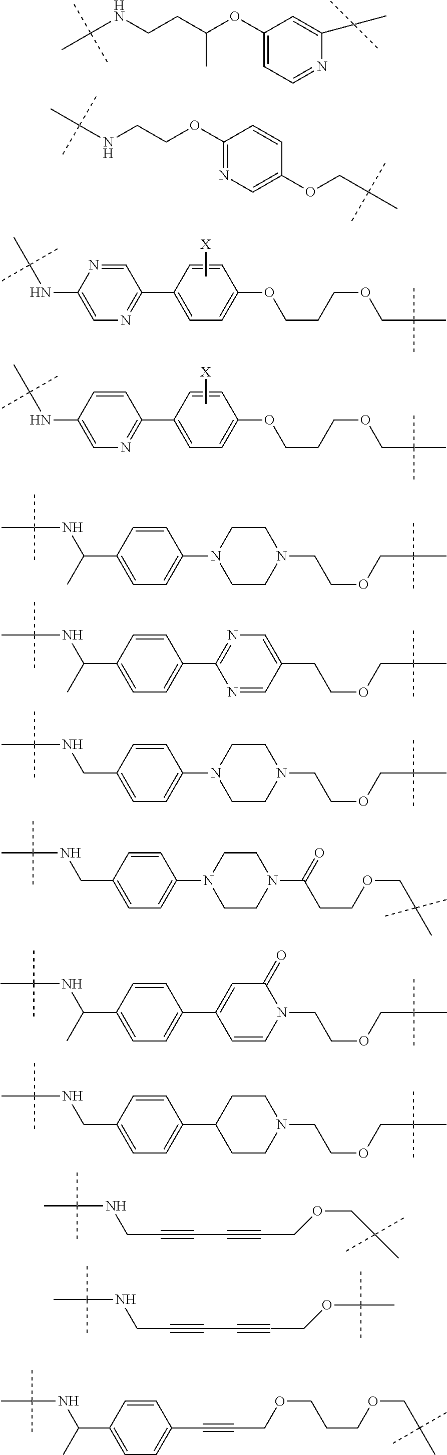

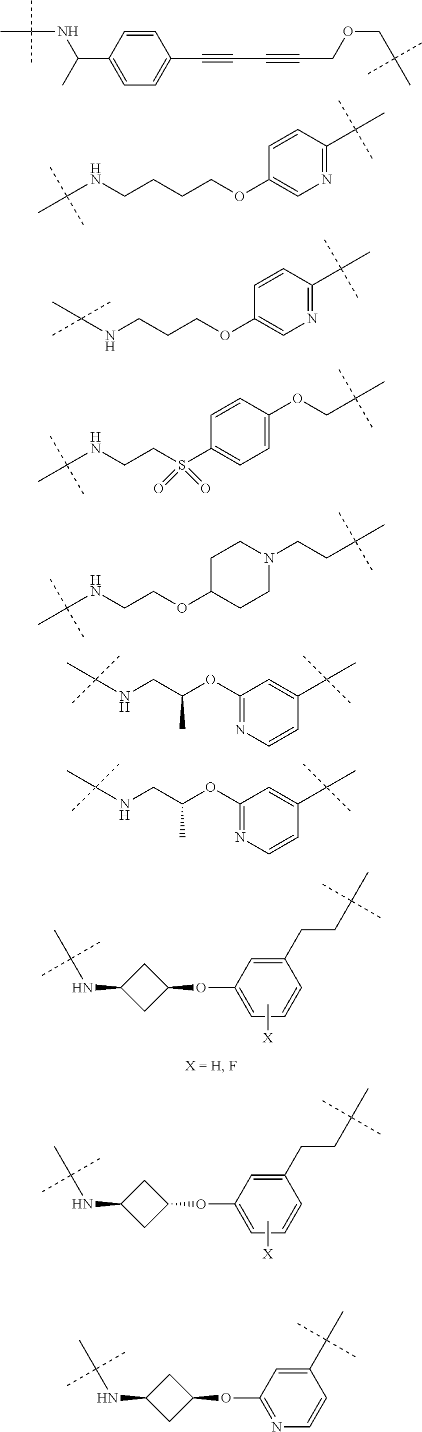

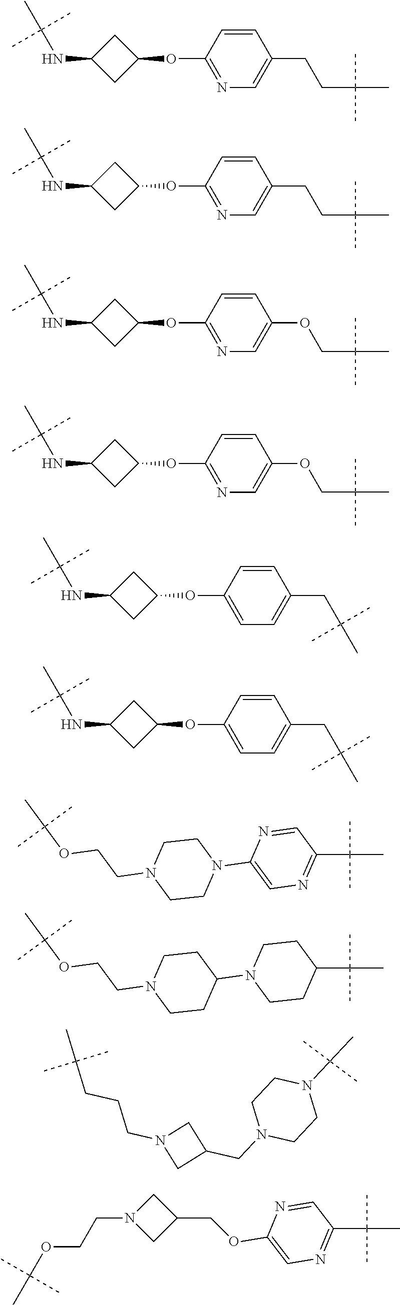

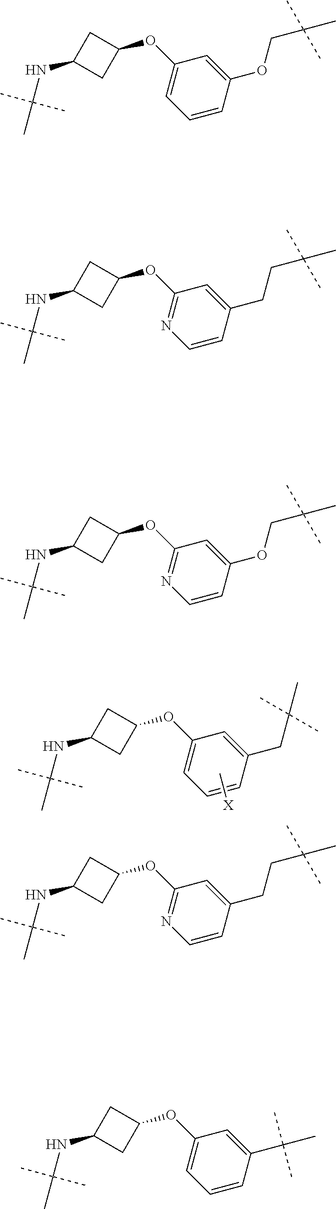

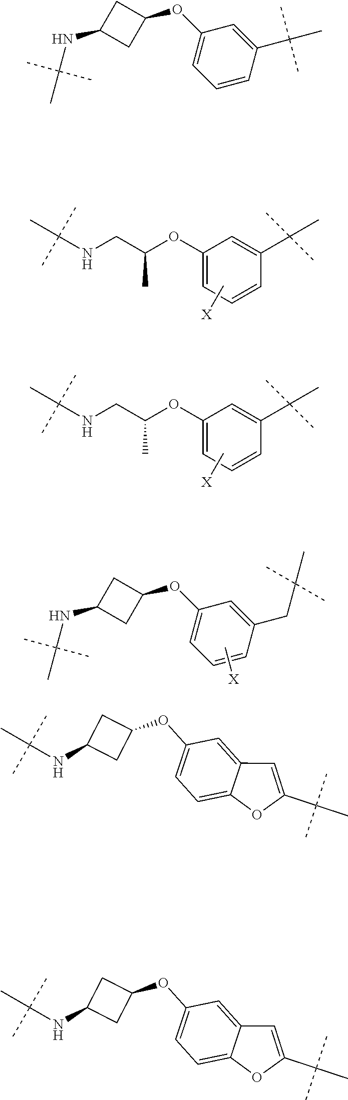

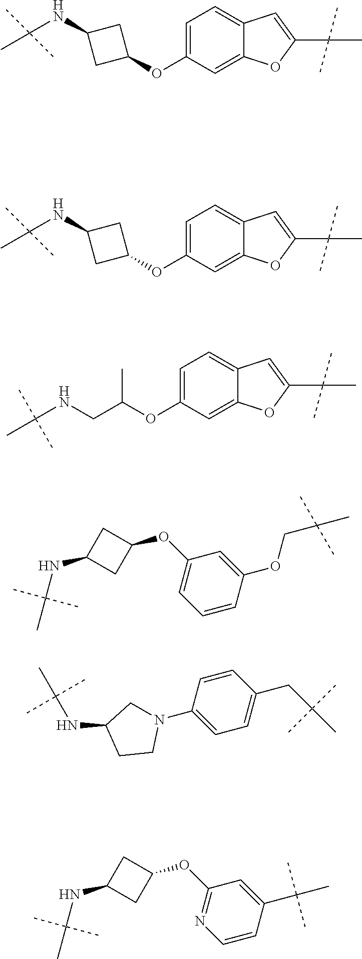

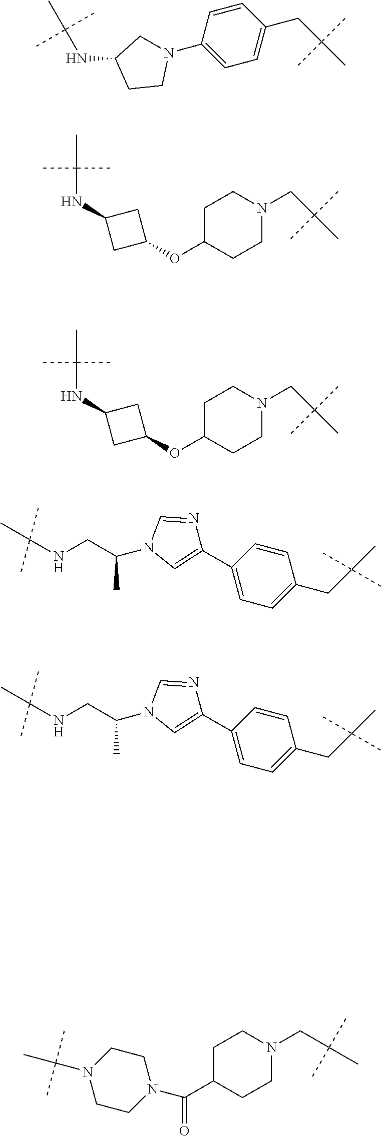

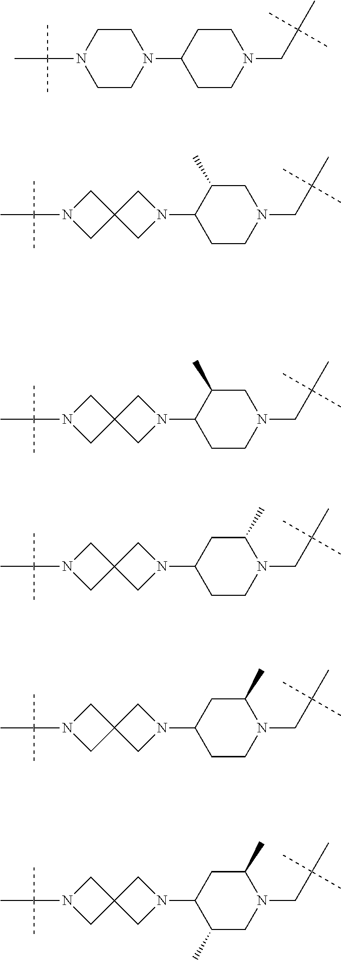

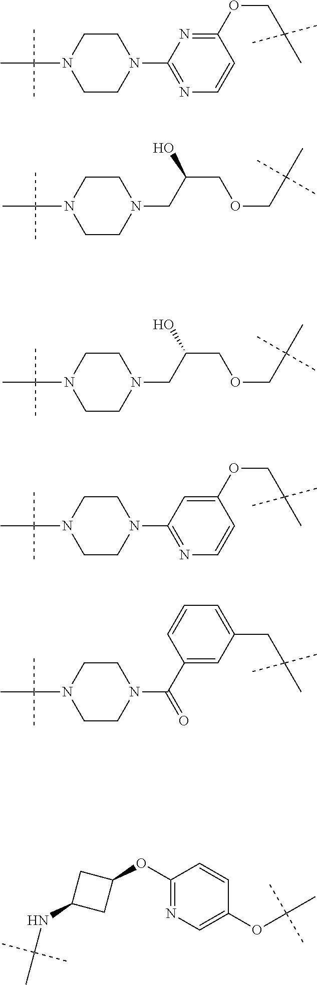

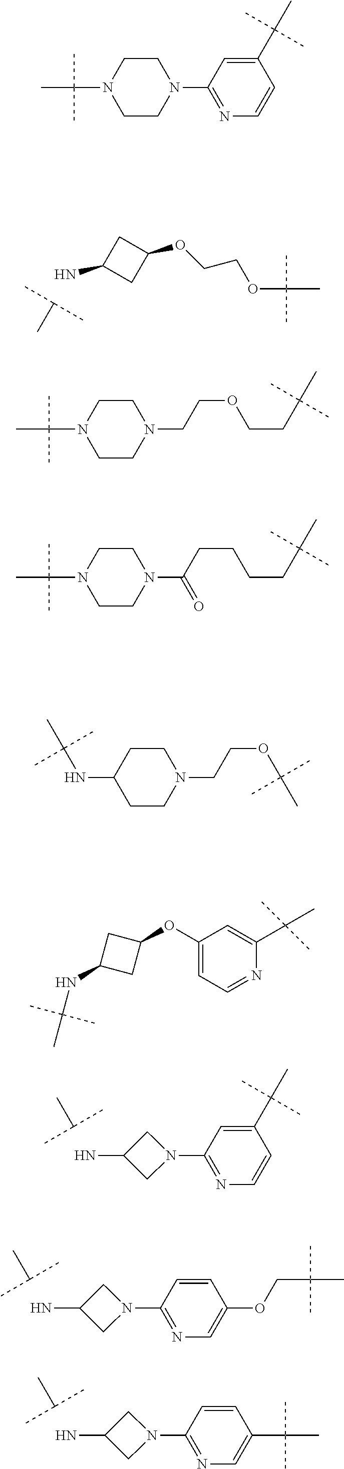

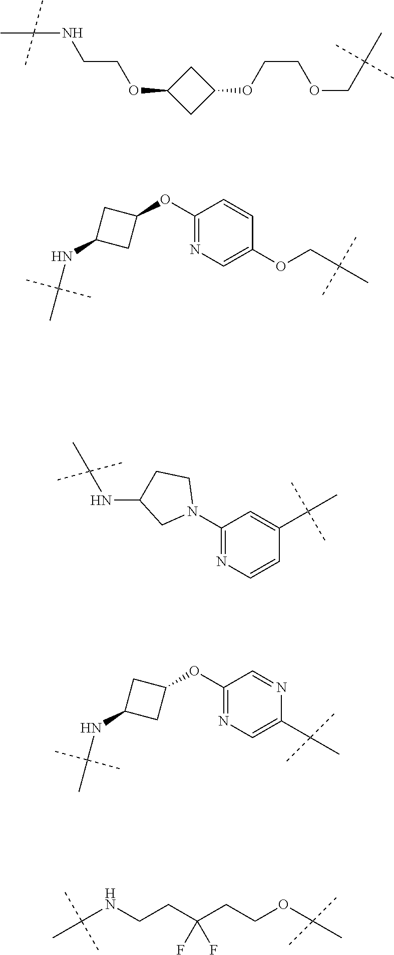

wherein any m or n are independently 0, 1, 2, 3, 4, 5, or 6; and any X is H or F.

In certain embodiments, L is one of the following:

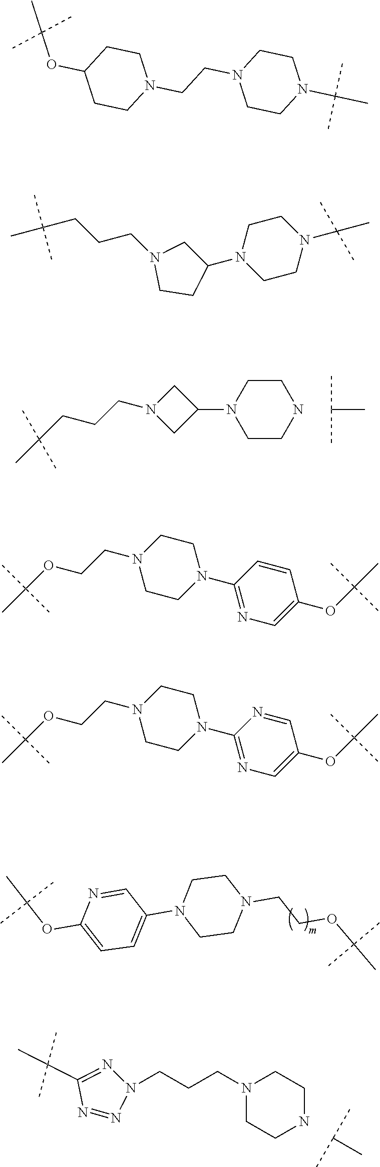

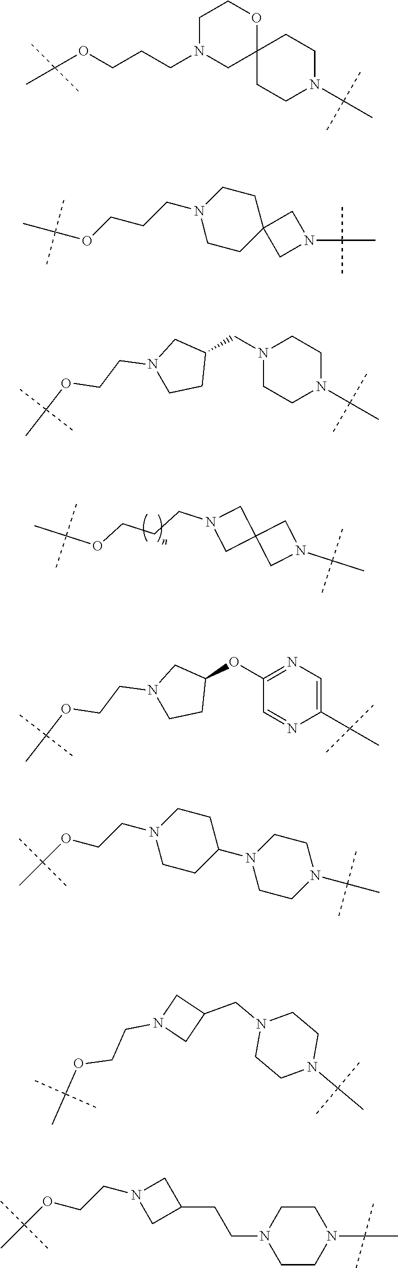

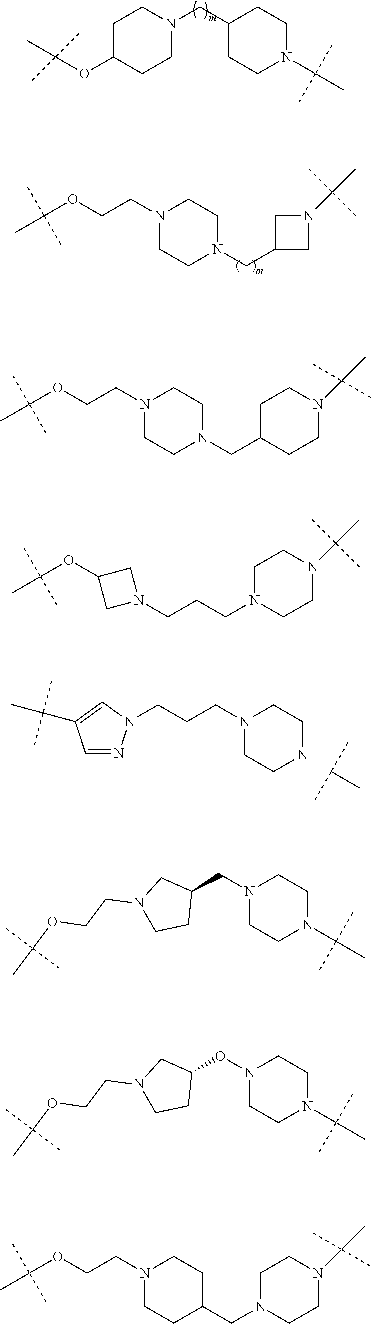

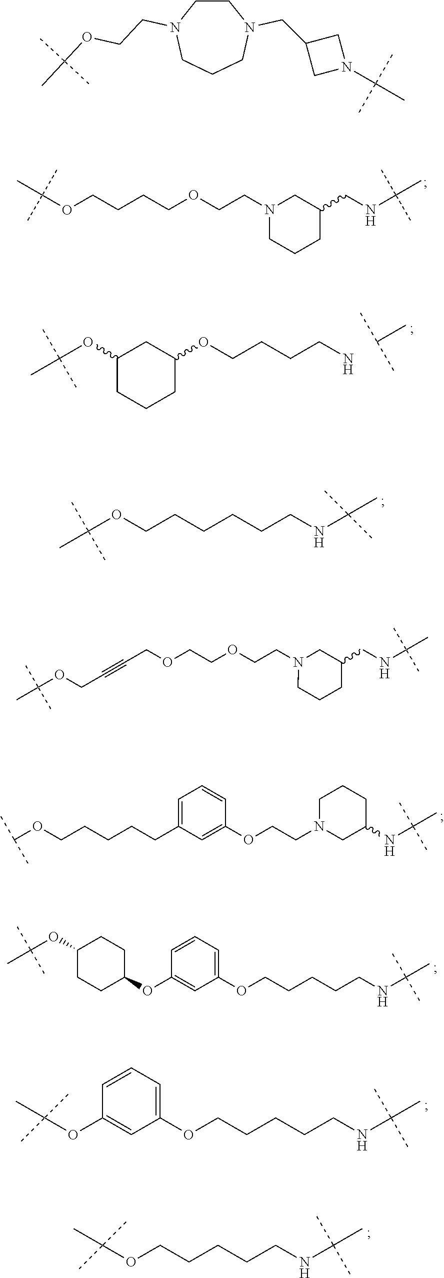

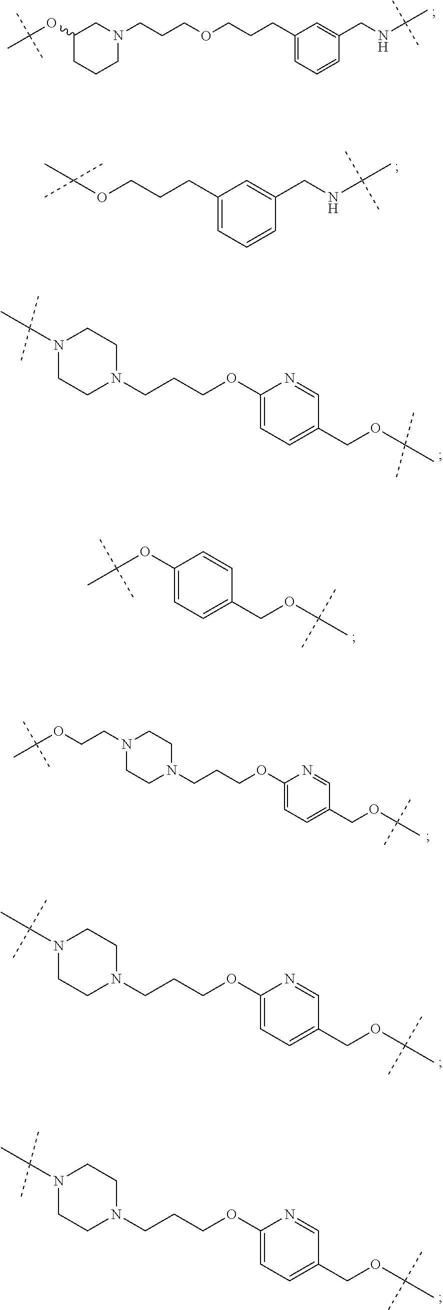

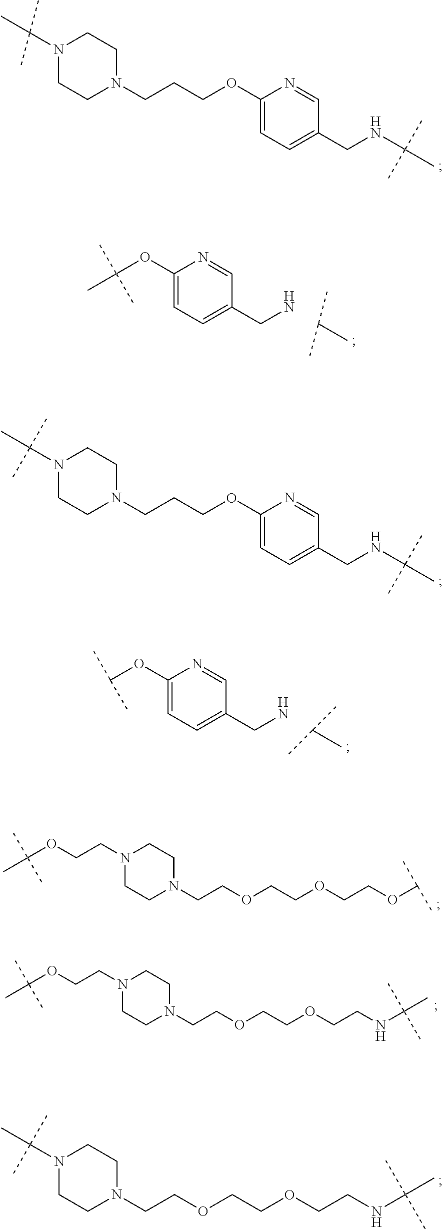

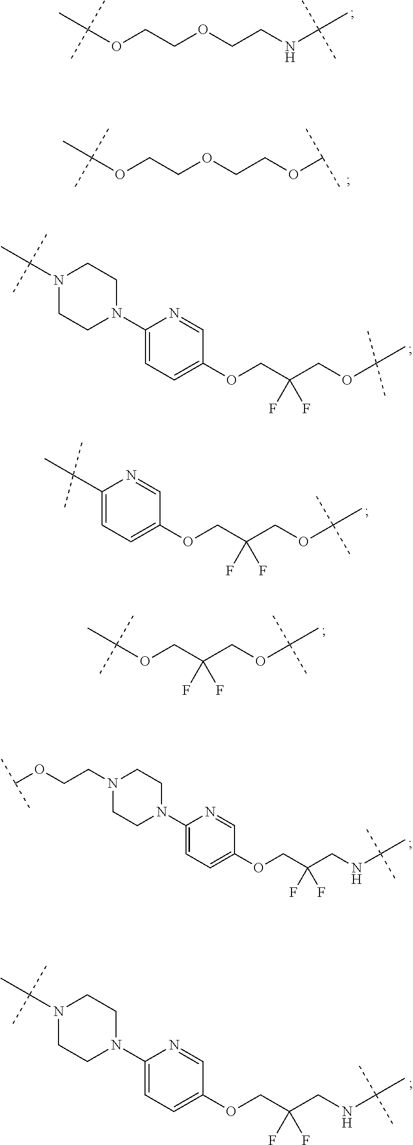

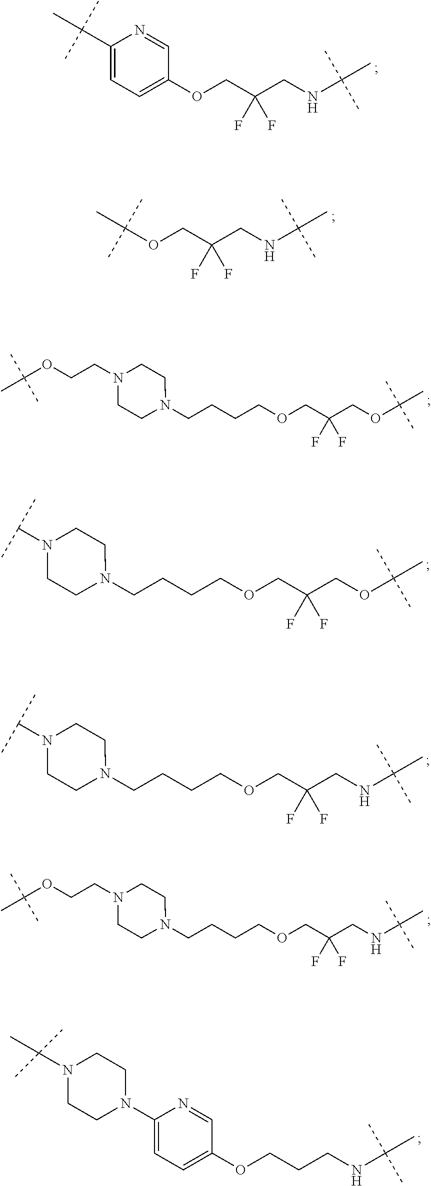

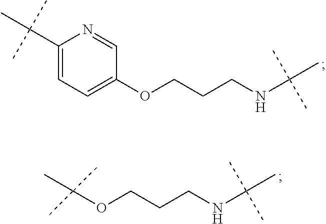

wherein any m or n are independently 0, 1, 2, 3, 4, 5, or 6.

In certain embodiments, L is one of the following:

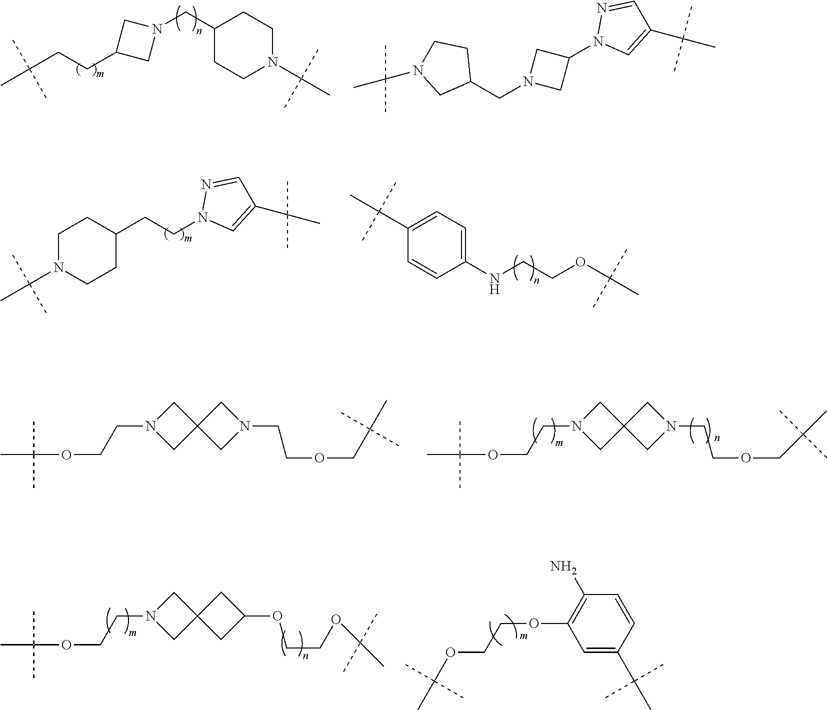

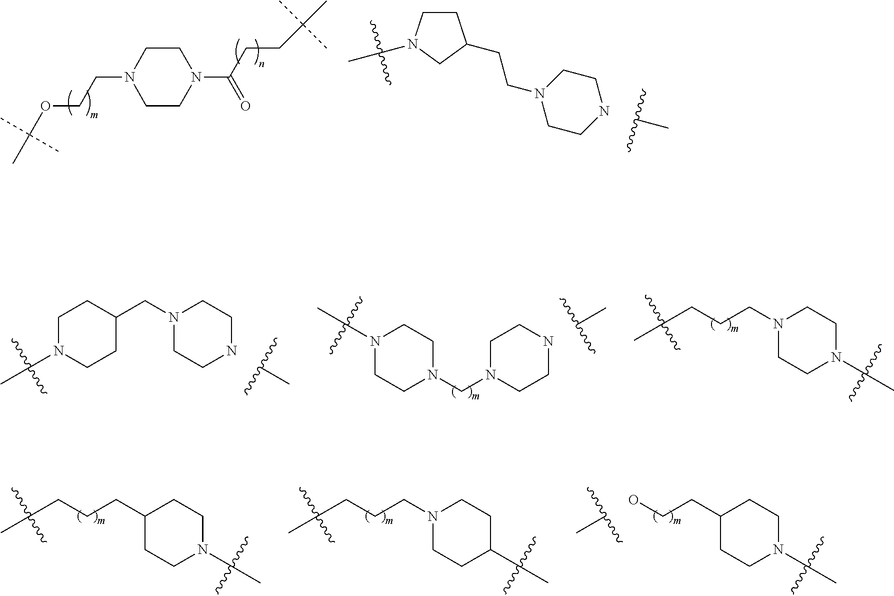

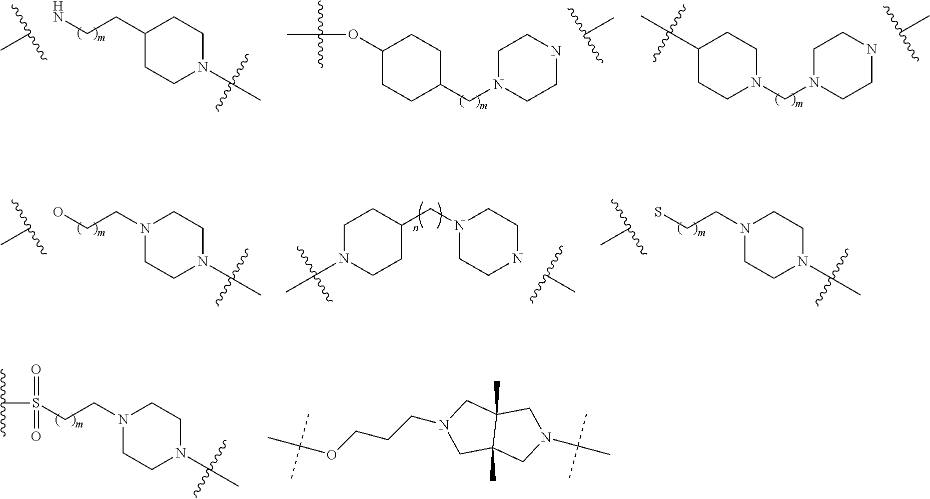

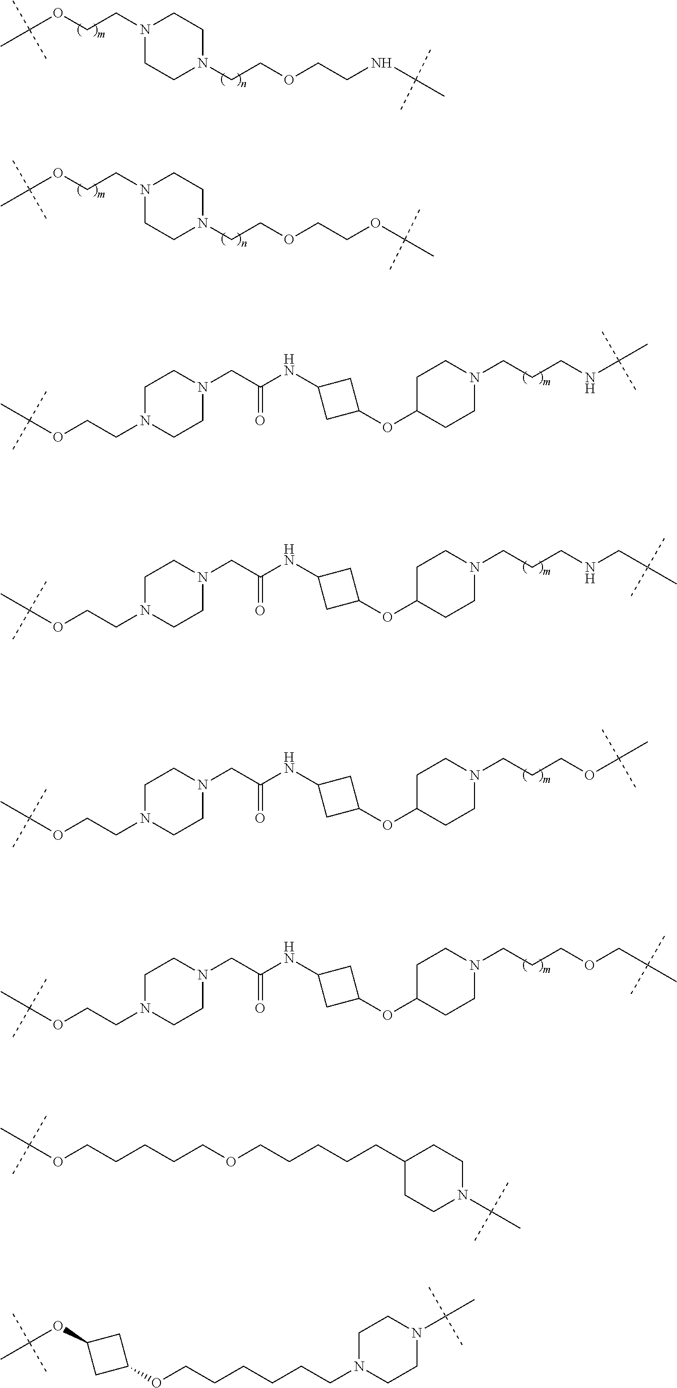

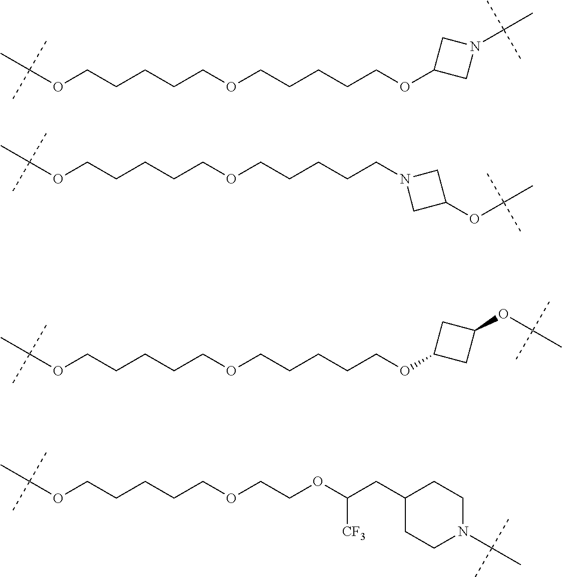

wherein any m or n are independently 0, 1, 2, 3, 4, 5, or 6.

In certain embodiments, L is one of the following:

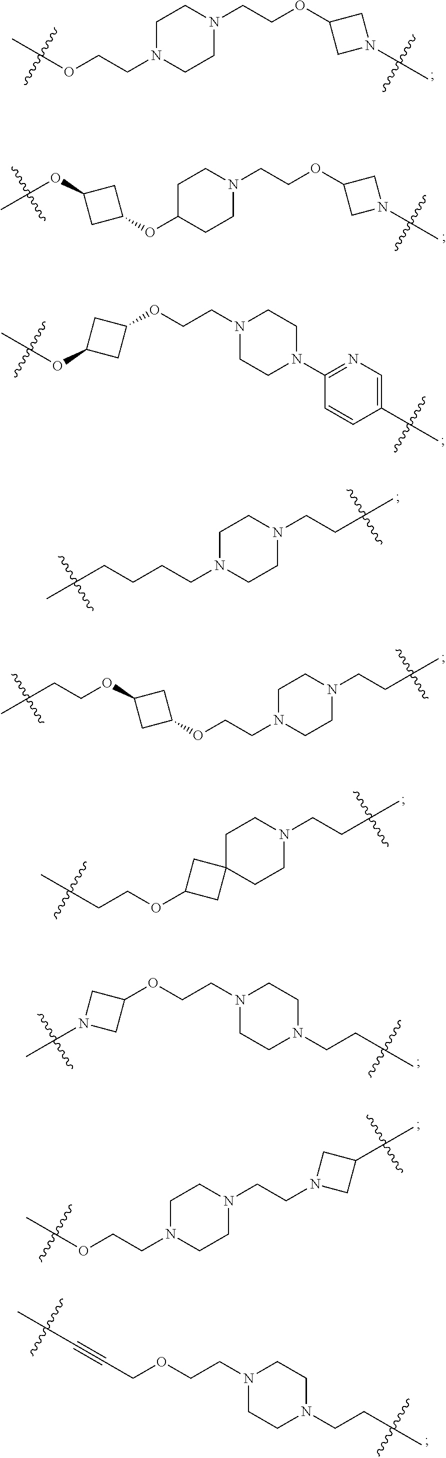

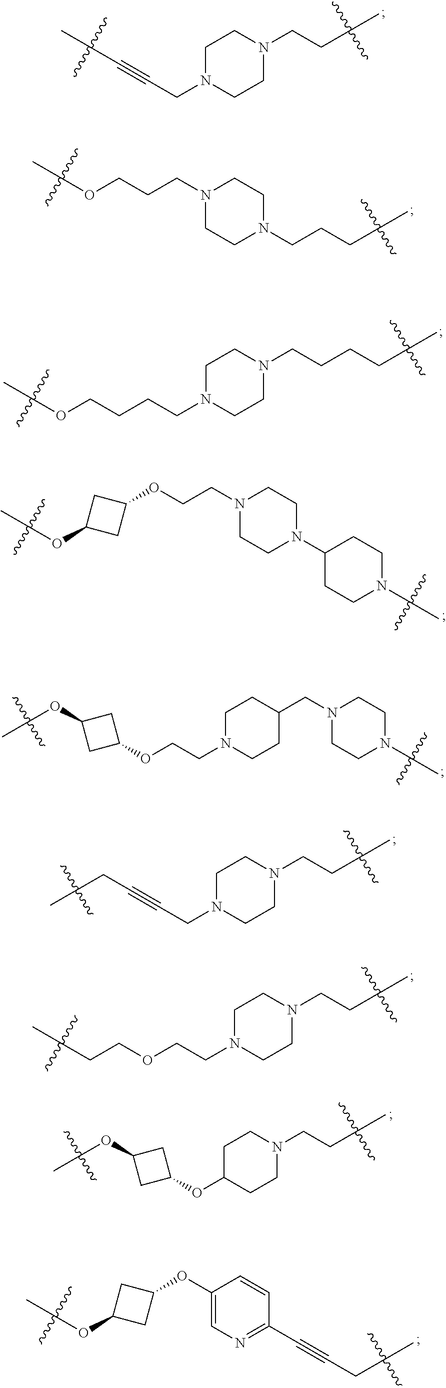

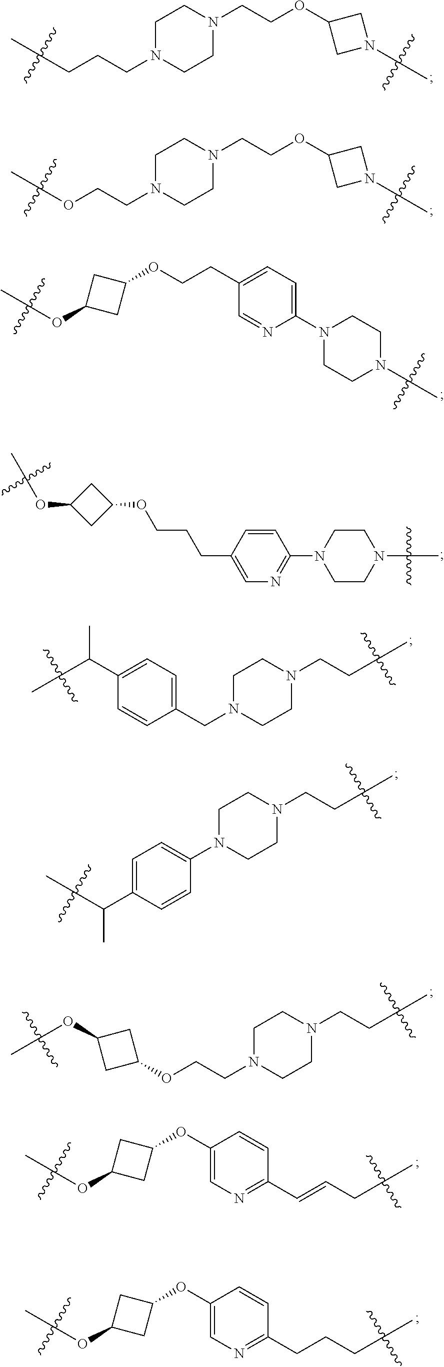

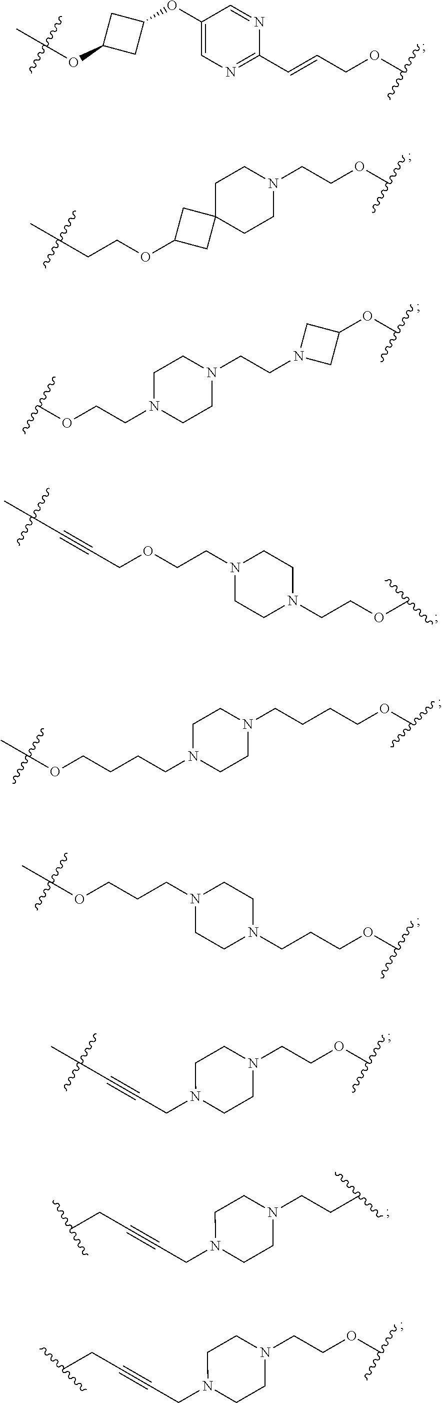

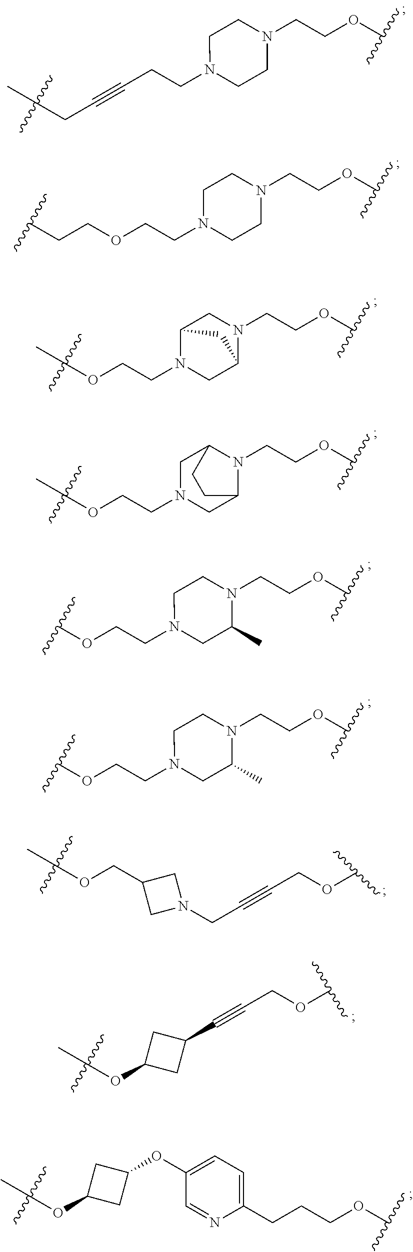

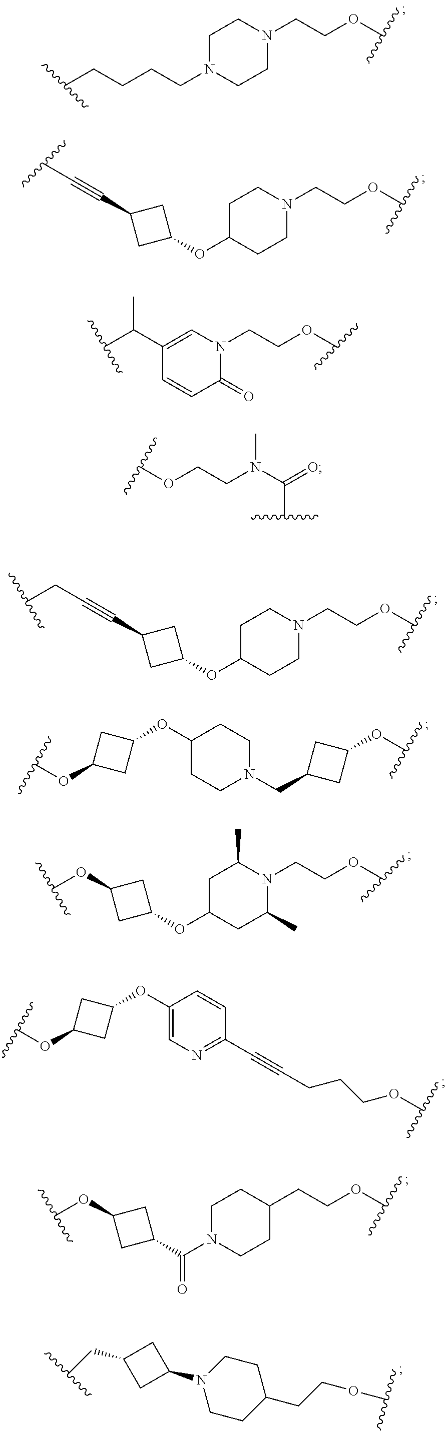

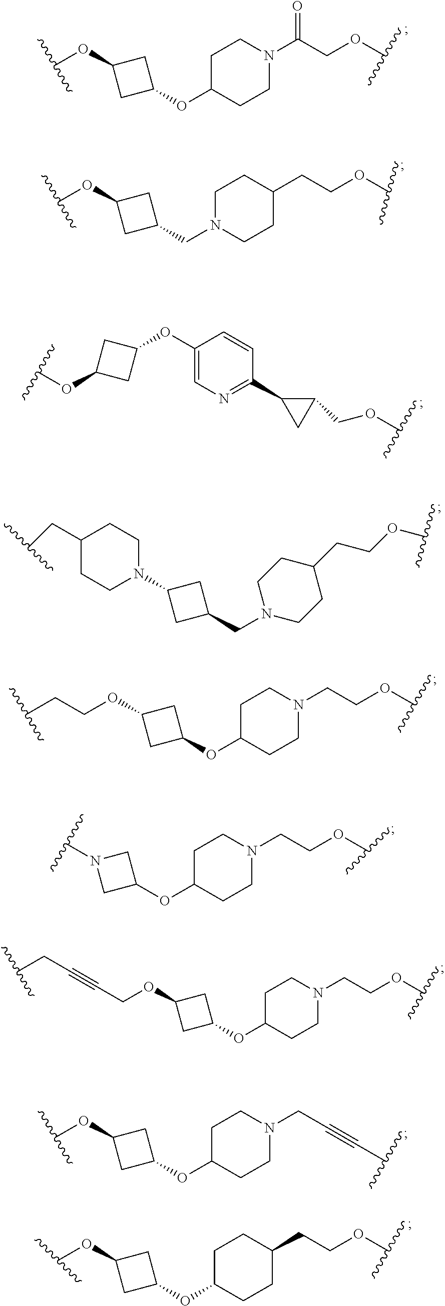

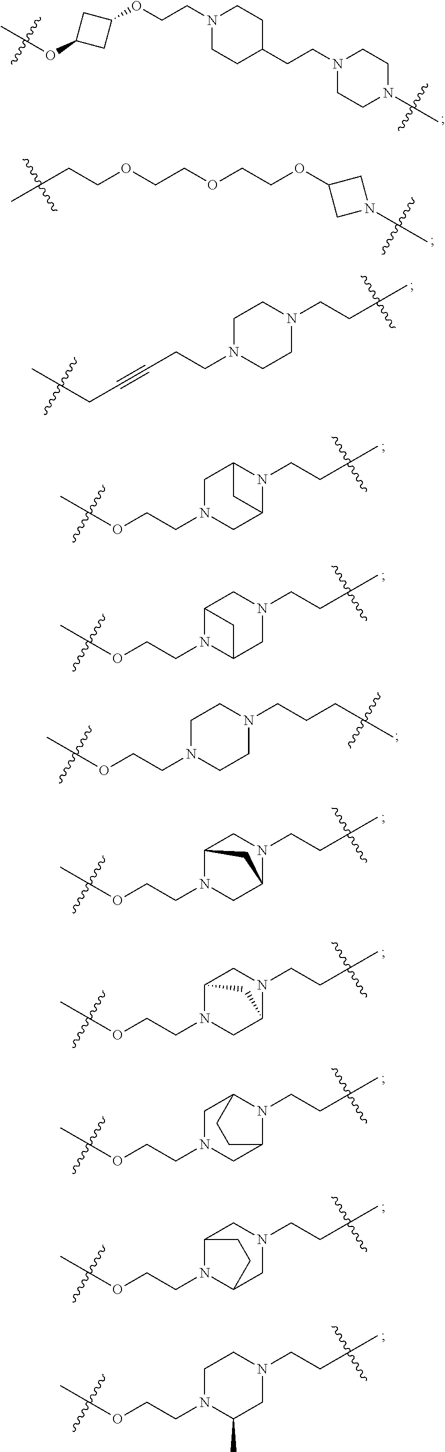

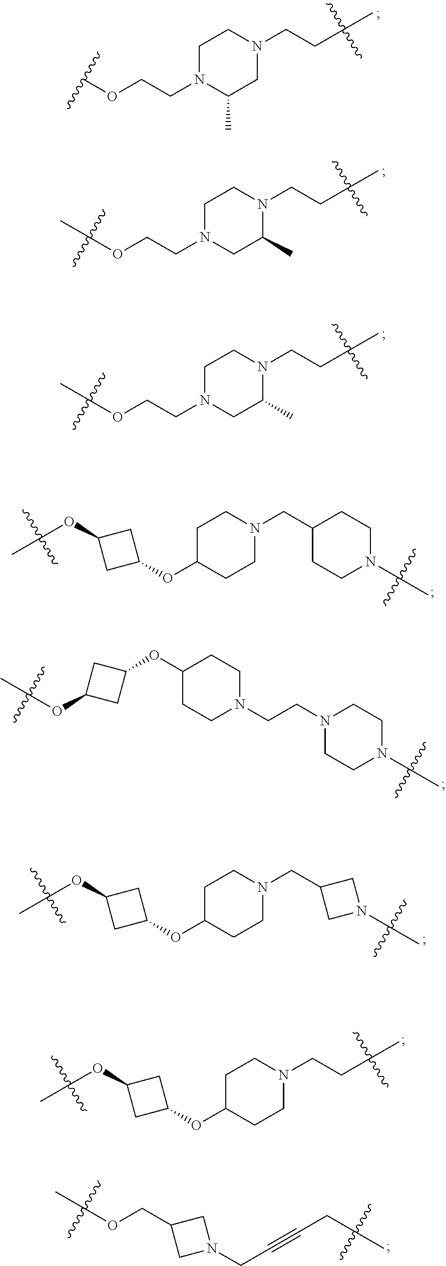

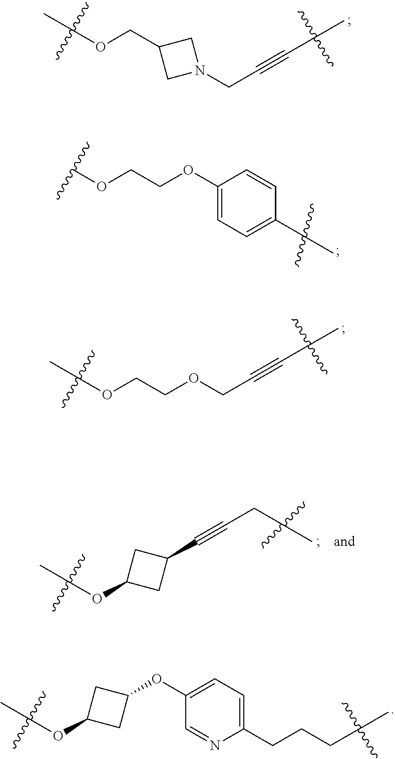 [SMILES: *C#CCN1CC(CO*)C1 ; *OCCOc1ccc(*)cc1 ; *C#CCOCCO* ; *CC#C[C@H]1C[C@@H](O*)C1 ; *CCCc1ccc(O[C@H]2C[C@H](O*)C2)cn1]

In certain embodiments, L is one of the following:

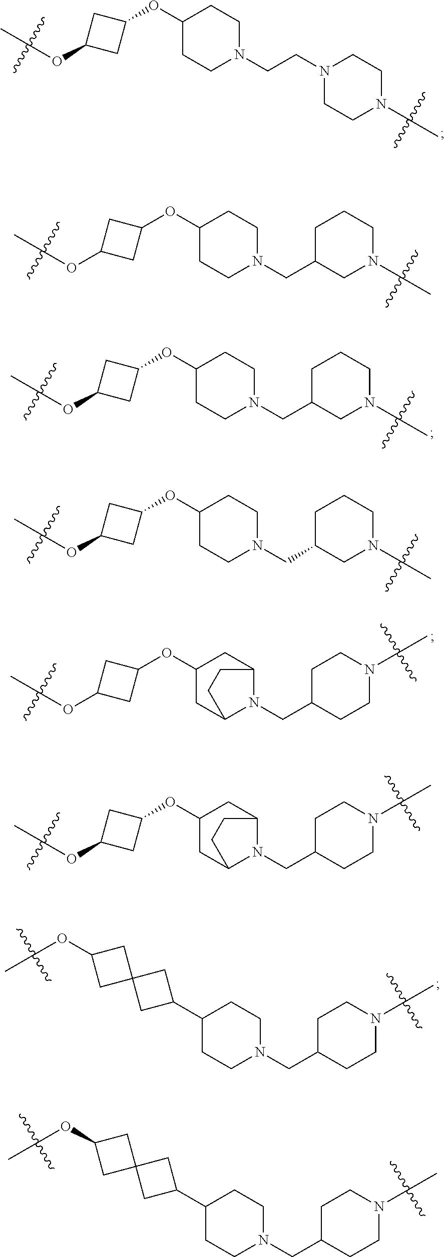

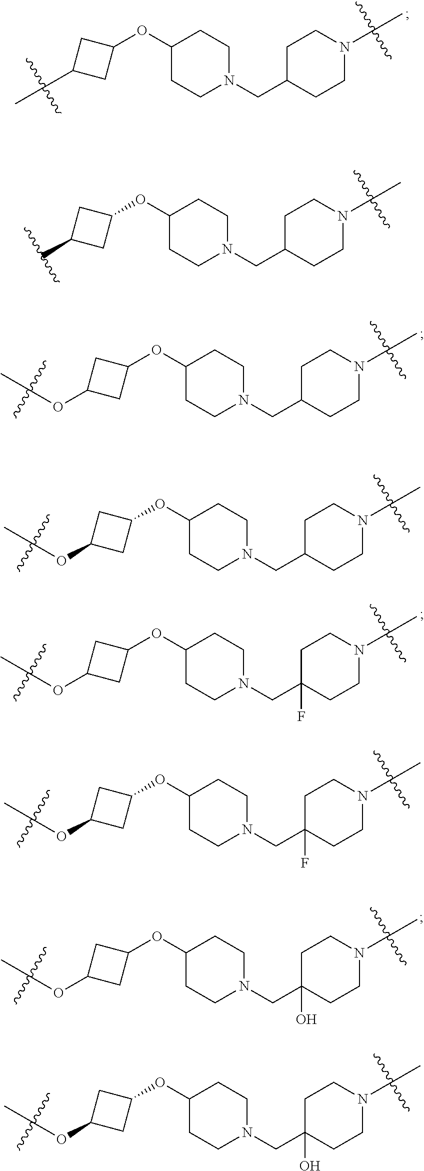

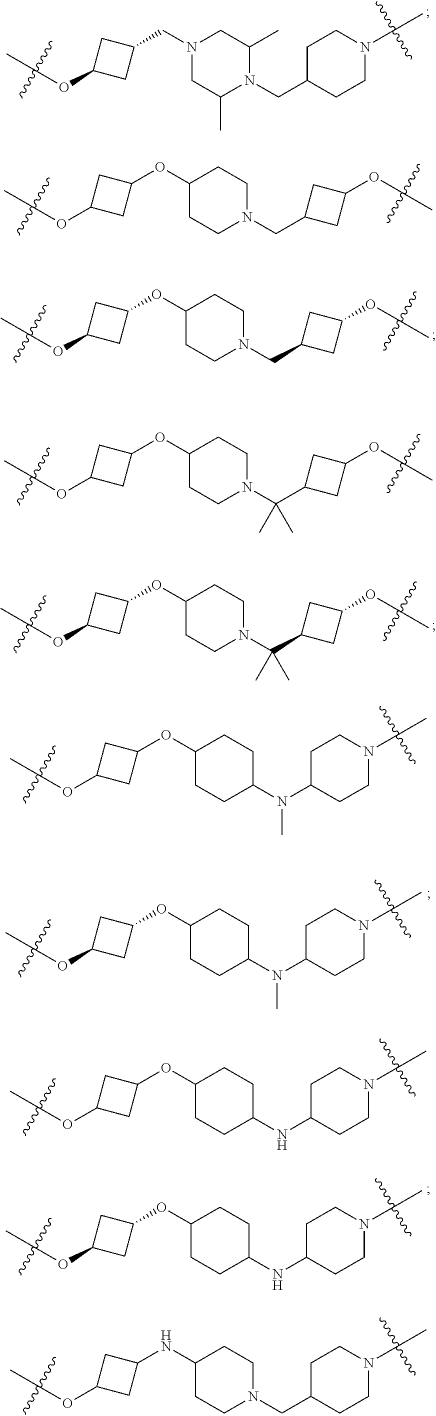

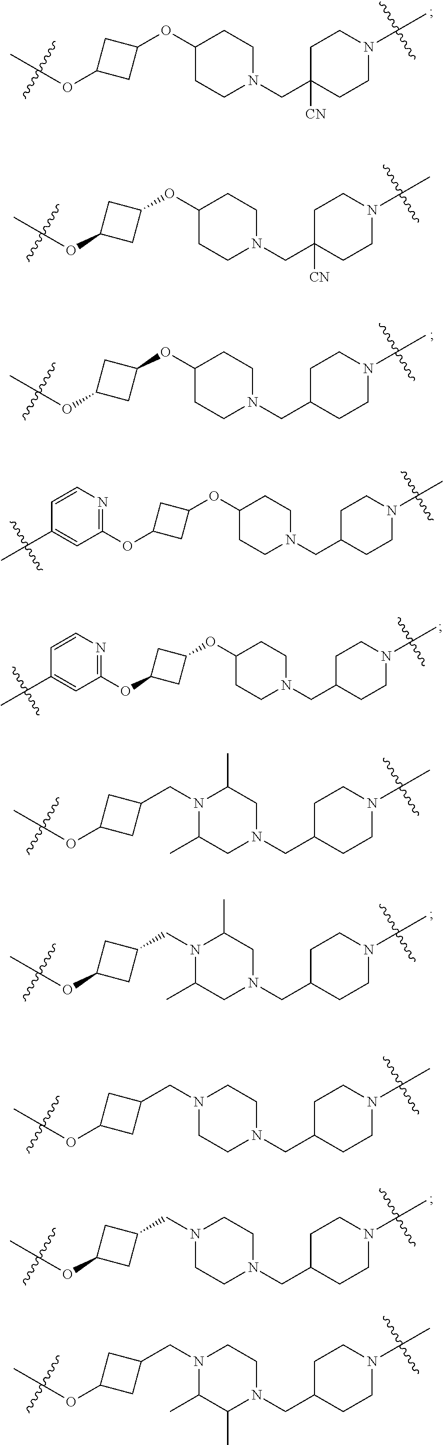

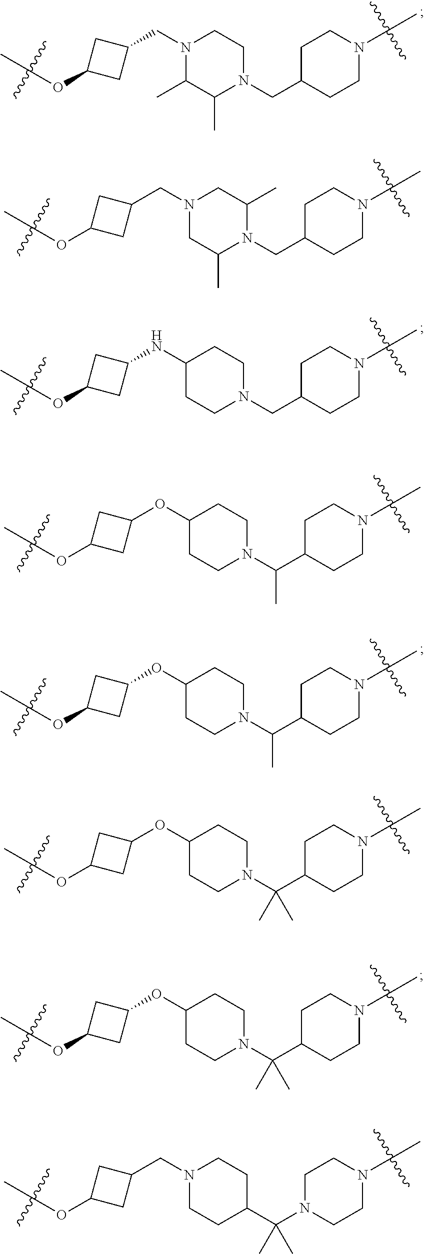

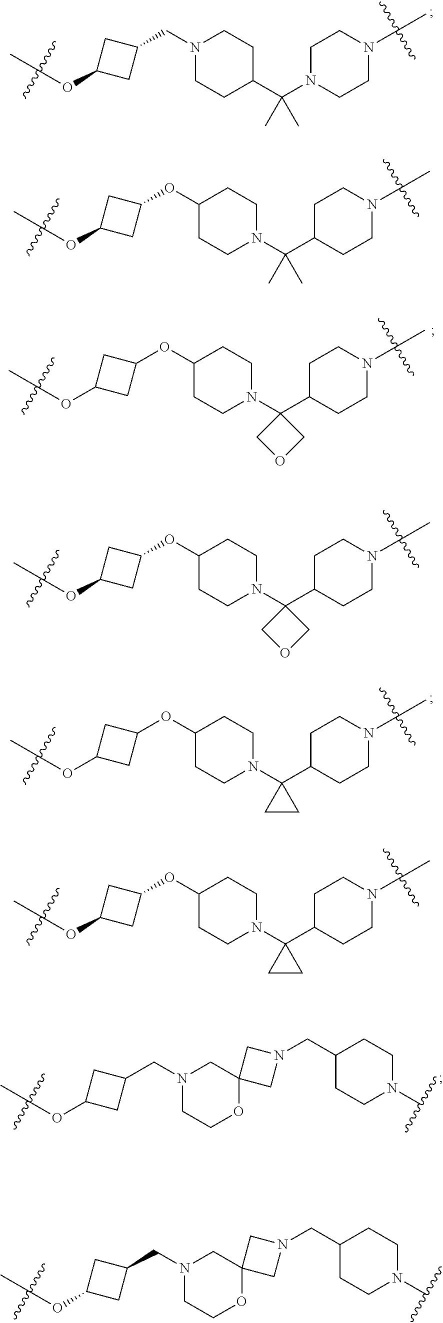

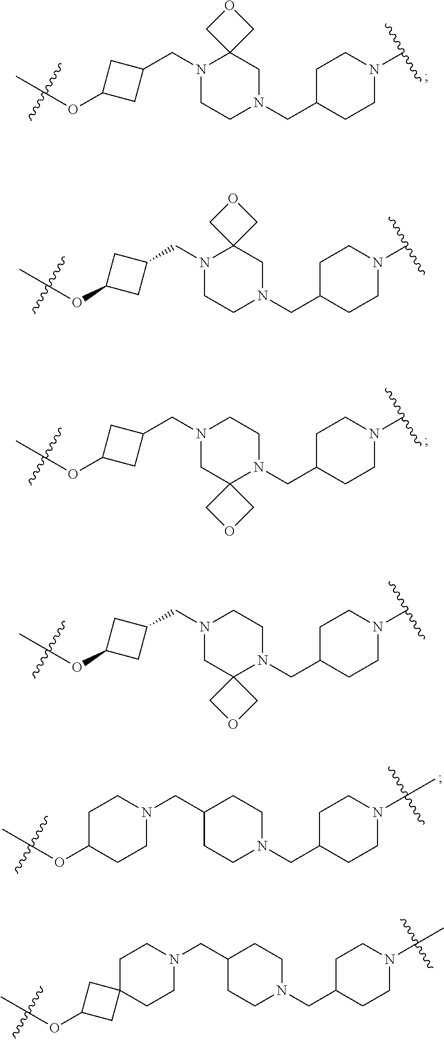 [SMILES: *OC1CC(CN2CCN(CC3CCN(*)CC3)CC23COC3)C1 ; *O[C@H]1C[C@H](CN2CCN(CC3CCN(*)CC3)CC23COC3)C1 ; *OC1CC(CN2CCN(CC3CCN(*)CC3)C3(COC3)C2)C1 ; *O[C@H]1C[C@H](CN2CCN(CC3CCN(*)CC3)C3(COC3)C2)C1 ; *OC1CCN(CC2CCN(CC3CCN(*)CC3)CC2)CC1 ; *OC1CC2(CCN(CC3CCN(CC4CCN(*)CC4)CC3)CC2)C1]

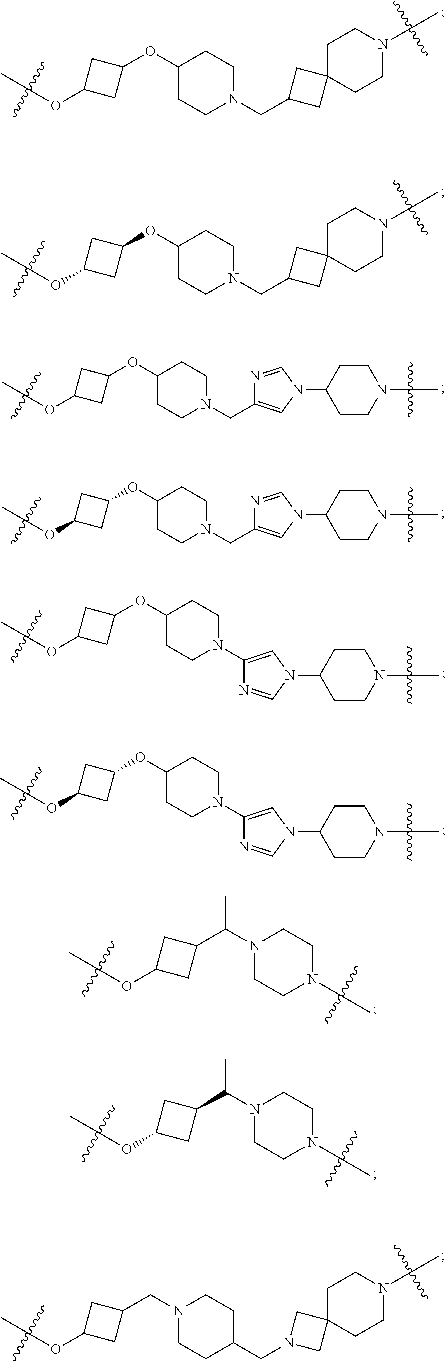

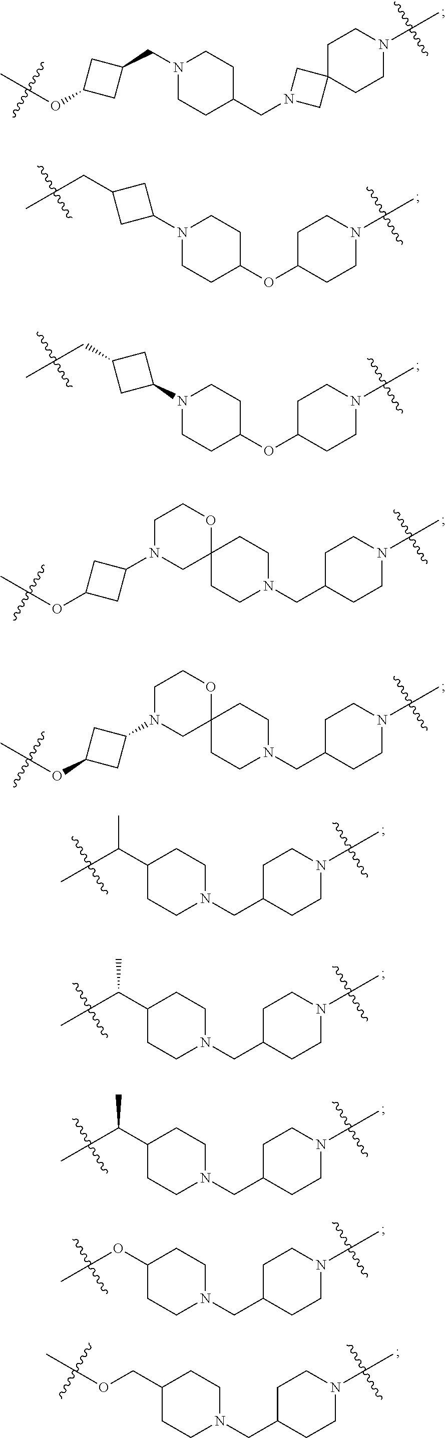

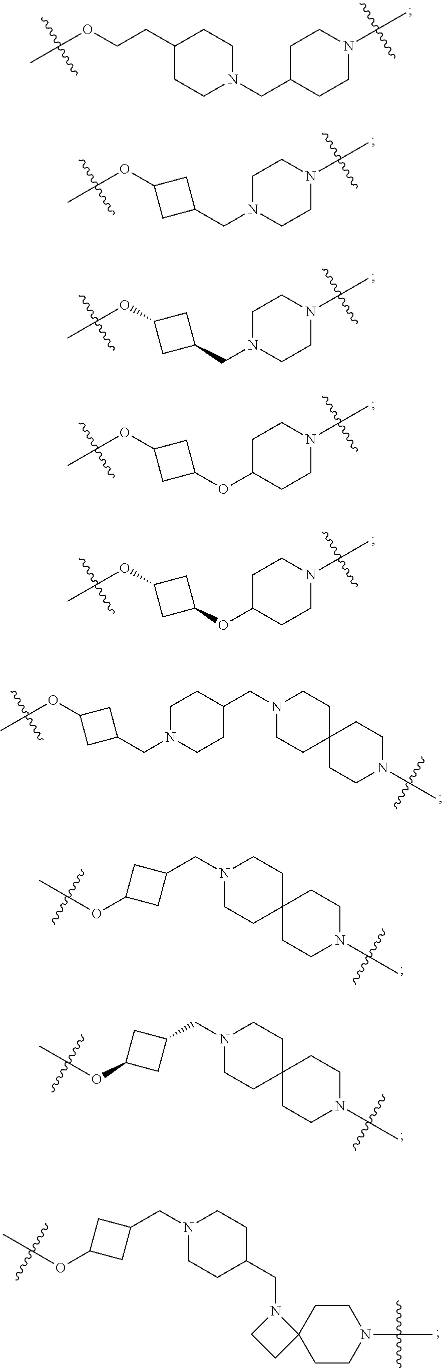

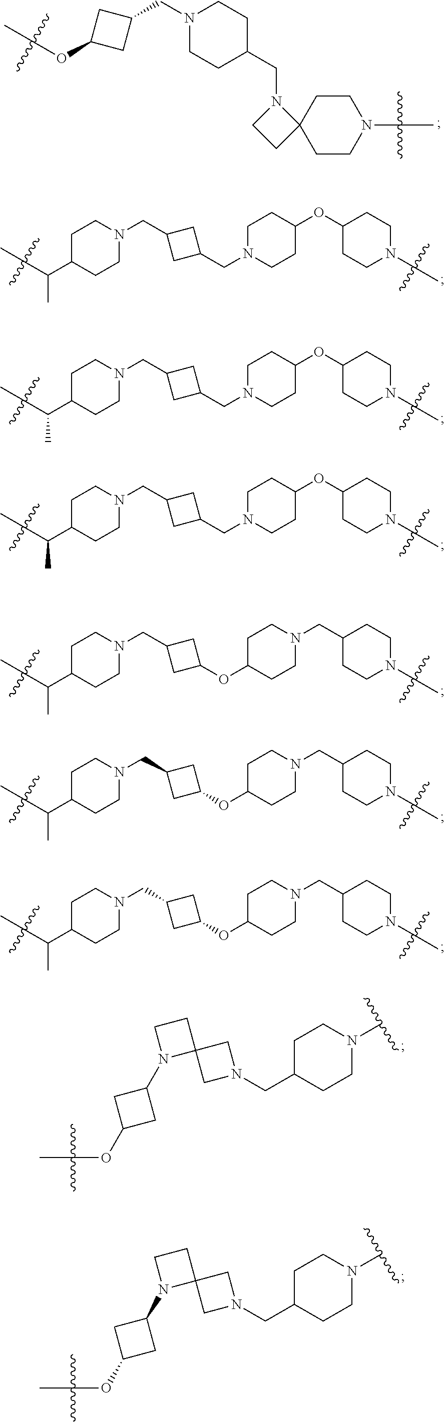

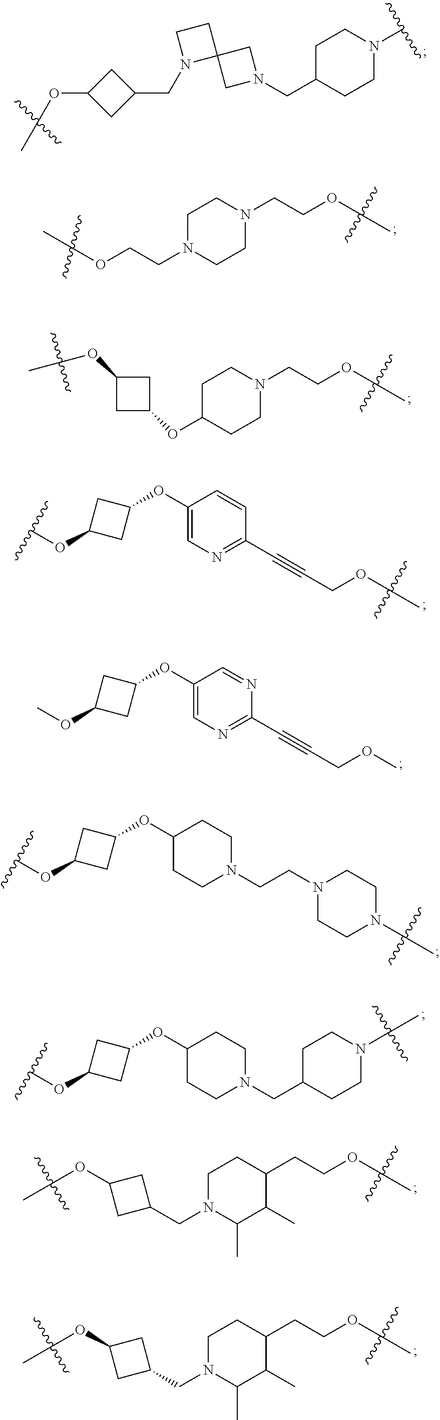

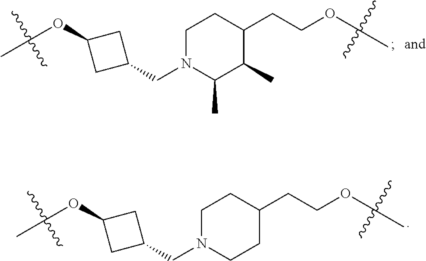 [SMILES: *OCCC1CCN(C[C@H]2C[C@H](O*)C2)[C@H](C)[C@@H]1C ; *OCCC1CCN(C[C@H]2C[C@H](O*)C2)CC1]

In certain embodiments, L has the formula —(C₀₋₁₂ alkylene)-(optionally substituted 3-40 membered heteroalkylene)-(C₀₋₁₂ alkylene)-. In certain embodiments, L is C₄₋₁₄ alkylene. In certain embodiments, L is —(CH₂)₆₋₁₀—.

In certain embodiments, L is —CH₂CH₂(OCH₂CH₂)—***, —CH₂CH₂(OCH₂CH₂)₂—***, —CH₂CH₂(OCH₂CH₂)₃—***, —CH₂CH₂(OCH₂CH₂)₄—***, —CH₂CH₂(OCH₂CH₂)₅—***, —CH₂CH₂(OCH₂CH₂)₆—***, —CH₂CH₂(OCH₂CH₂)₇—***, —CH₂CH₂(OCH₂CH₂)₈—***, —CH₂CH₂(OCH₂CH₂)₉—***, —CH₂CH₂(OCH₂CH₂)₁₀—***, —CH₂CH₂(OCH₂CH₂)ₙ1-***, —CH₂CH₂(OCH₂CH₂)₁₂—***, —CH₂CH₂(OCH₂CH₂)₁₃—***, —CH₂CH₂(OCH₂CH₂)₁₄—***, —CH₂CH₂(OCH₂CH₂)₁₅—***, or —CH₂CH₂(OCH₂CH₂)₁₆₋₂₀—***, where *** is a point of attachment to A².

In certain embodiments, L is —(C₂₋₂₀ alkylene)-(OCH₂CH₂)₂₋₄—(C₀₋₄ alkylene)-***, —(C₂-20 alkylene)-(OCH₂CH₂)₅₋₇—(C₀₋₄ alkylene)-***, —(C₂₋₂₀ alkylene)-(OCH₂CH₂)₅₋₁₀—(C₀₋₄ alkylene)-***, —(C₂₋₂₀ alkylene)-(OCH₂CH₂)₁₋₁₃—(C₀₋₄ alkylene)-***, —(C₂₋₂₀ alkylene)-(OCH₂CH₂)₁₄₋₁₆—(C₀₋₄ alkylene)-***, —(C₂₋₂₀ alkylene)-(OCH₂CH₂)₁₇₋₂₀—(C₀₋₄ alkylene)-***, —(C₁₋₂₀ alkylene)-(OCH₂CH₂)₁₋₁₀—(C₀₋₄ alkylene)-C(O)—***, or —(C₁₋₂₀ alkylene)-(OCH₂CH₂)₁₆₋₂₀—(C₀₋₄ alkylene)-C(O)—***, where *** is a point of attachment to A².

In certain embodiments, L is —O(CH₂CH₂O)₋₄—(C₀₋₄ alkylene)-***, —O(CH₂CH₂O)₅0.7—(C₀₋₄ alkylene)-***, —O(CH₂CH₂O)₅₋₁₀—(C₀₋₄ alkylene)-***, —O(CH₂CH₂O)ₙ3-(C₀₋₄ alkylene)-***, —O(CH₂CH₂O)₁₄₋₁₆—(C₀₋₄alkylene)-***, —O(CH₂CH₂O)₁₆₋₂₀—(C₀₋₄ alkylene)-***, —O(CH₂CH₂O)₂₋₁₀—(C₀₋₄ alkylene)C(O)—***, or —O(CH₂CH₂O)₁₁₋₂₀—(C₀₋₄ alkylene)C(O)—***, where *** is a point of attachment to A².

In certain embodiments, L is —(C₀₋₂₀ alkylene)-(OCH₂CH₂)₁₋₁₀—(N(C₁₋₄ alkyl))-***, —(C₀₋₂₀ alkylene)-(OCH₂CH₂)₁₁₋₂₀—(N(C₁₋₄ alkyl))-***, —(C₀₋₂₀ alkylene)-(CH₂CH₂O)₁₋₁₀—(C₂₋₁₀ alkylene)-(N(C₁₋₄ alkyl))-(C₀₋₁₀ alkylene)-***, or —(C₀₋₂₀ alkylene)-(CH₂CH₂O)₁₁₋₂₀—(C₂₋₁₀ alkylene)-(N(C₁₋₄ alkyl))-(C₀₋₁₀ alkylene)-***, where *** is a point of attachment to A².

In certain embodiments, L is selected from those depicted in the compounds in Table 1, below.

### Exemplary Specific Compounds

In certain embodiments, the compound is a compound in Table 1, or a pharmaceutically acceptable salt thereof. In certain embodiments, the compound is a compound in Table 1. In certain embodiments, the compound is a compound selected from I-1 through I-103 of Table 1 below, or a pharmaceutically acceptable salt thereof. In certain embodiments, the compound is a compound selected from I-1 through I-103 of Table 1 below. In certain embodiments, the compound is a compound selected from I-1 through 323 of Table 1 below, or a pharmaceutically acceptable salt thereof. In certain embodiments, the compound is a compound selected from I-1 through I-323 of Table 1 below.

TABLE 1

| Com- | |
|---|---|
| pound | |
| No. | Chemical Structure |
| I-1 | 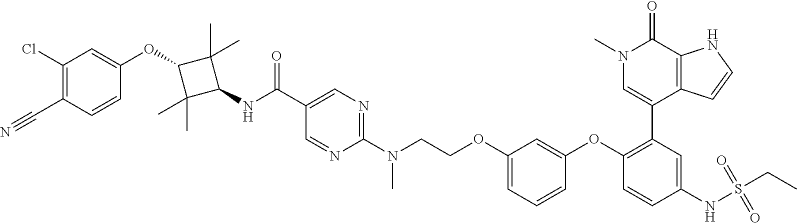 [SMILES: CCS(=O)(=O)Nc1ccc(Oc2cccc(OCCN(C)c3ncc(C(=O)N[C@H]4C(C)(C)[C@H](Oc5ccc(C#N)c(Cl)c5)C4(C)C)cn3)c2)c(-c2cn(C)c(=O)c3[nH]ccc23)c1] |
| I-2 | 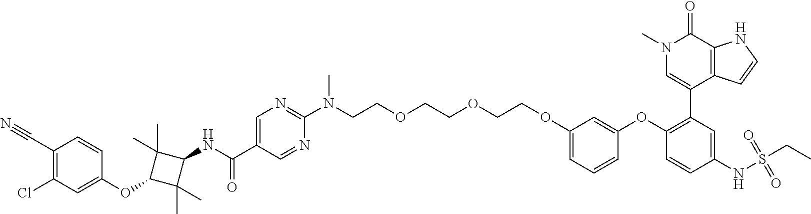 [SMILES: CCS(=O)(=O)Nc1ccc(Oc2cccc(OCCOCCOCCN(C)c3ncc(C(=O)N[C@H]4C(C)(C)[C@H](Oc5ccc(C#N)c(Cl)c5)C4(C)C)cn3)c2)c(-c2cn(C)c(=O)c3[nH]ccc23)c1] |
| I-3 | 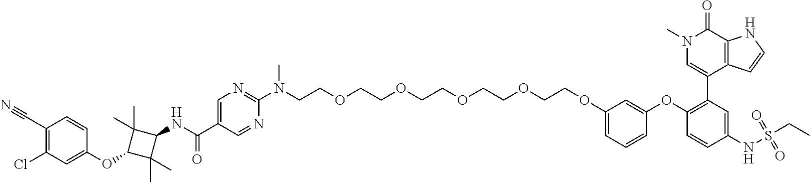 [SMILES: CCS(=O)(=O)Nc1ccc(Oc2cccc(OCCOCCOCCOCCOCCN(C)c3ncc(C(=O)N[C@H]4C(C)(C)[C@H](Oc5ccc(C#N)c(Cl)c5)C4(C)C)cn3)c2)c(-c2cn(C)c(=O)c3[nH]ccc23)c1] |
| I-4 | 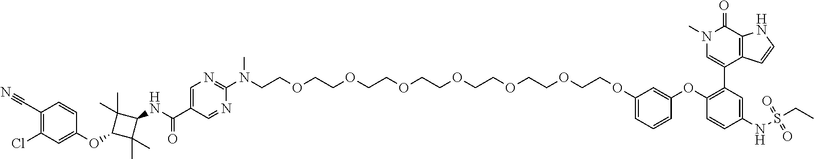 [SMILES: CCS(=O)(=O)Nc1ccc(Oc2cccc(OCCOCCOCCOCCOCCOCCOCCN(C)c3ncc(C(=O)N[C@H]4C(C)(C)[C@H](Oc5ccc(C#N)c(Cl)c5)C4(C)C)cn3)c2)c(-c2cn(C)c(=O)c3[nH]ccc23)c1] |
| I-5 | 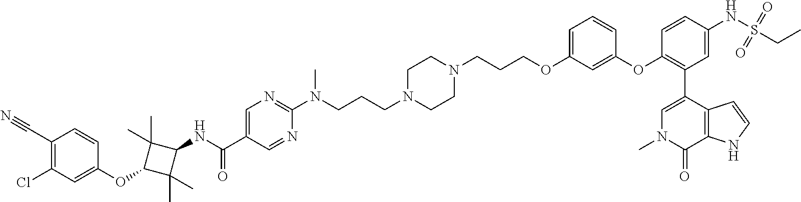 [SMILES: CCS(=O)(=O)Nc1ccc(Oc2cccc(OCCCN3CCN(CCCN(C)c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-6 | 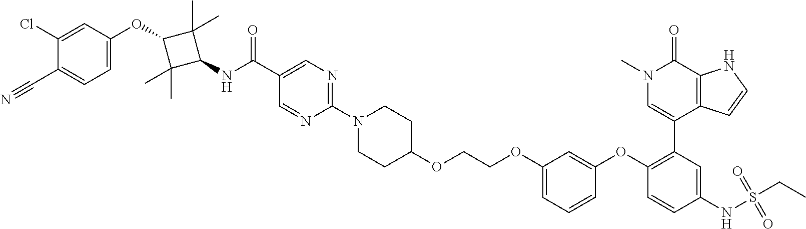 [SMILES: CCS(=O)(=O)Nc1ccc(Oc2cccc(OCCOC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-7 | 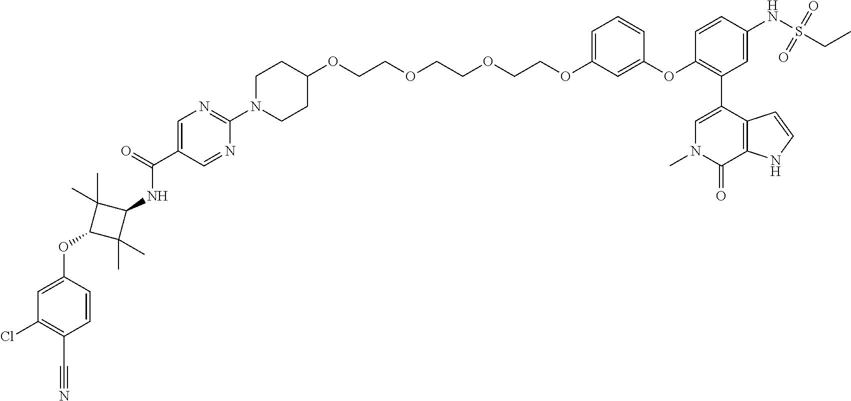 [SMILES: CCS(=O)(=O)Nc1ccc(Oc2cccc(OCCOCCOCCOC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-8 | 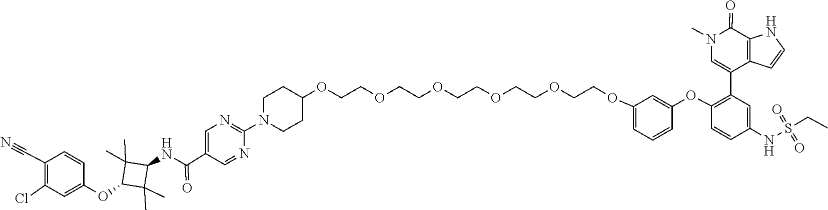 [SMILES: CCS(=O)(=O)Nc1ccc(Oc2cccc(OCCOCCOCCOCCOCCOC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-9 | 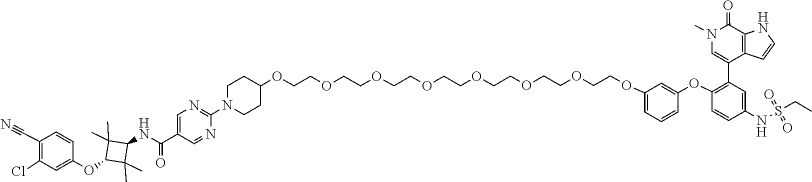 [SMILES: CCS(=O)(=O)Nc1ccc(Oc2cccc(OCCOCCOCCOCCOCCOCCOCCOC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-10 | 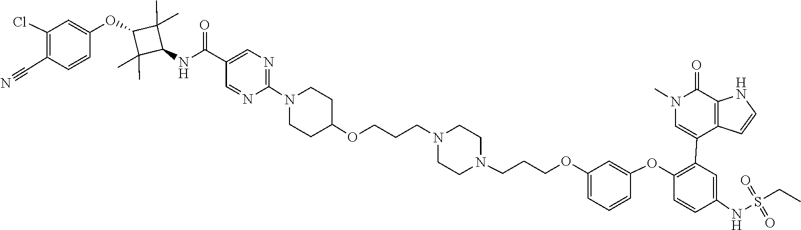 [SMILES: CCS(=O)(=O)Nc1ccc(Oc2cccc(OCCCN3CCN(CCCOC4CCN(c5ncc(C(=O)N[C@H]6C(C)(C)[C@H](Oc7ccc(C#N)c(Cl)c7)C6(C)C)cn5)CC4)CC3)c2)c(-c2cn(C)c(=O)c3[nH]ccc23)c1] |
| I-11 | 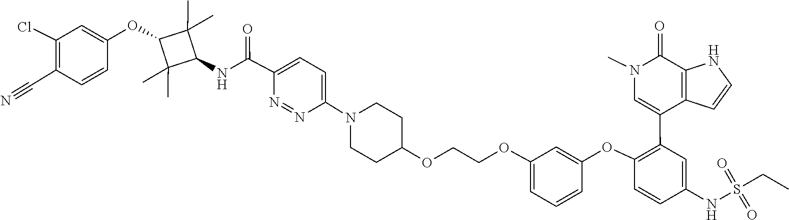 [SMILES: CCS(=O)(=O)Nc1ccc(Oc2cccc(OCCOC3CCN(c4ccc(C(=O)N[C@H]5C(C)(C)[C@H](Oc6ccc(C#N)c(Cl)c6)C5(C)C)nn4)CC3)c2)c(-c2cn(C)c(=O)c3[nH]ccc23)c1] |
| I-12 | 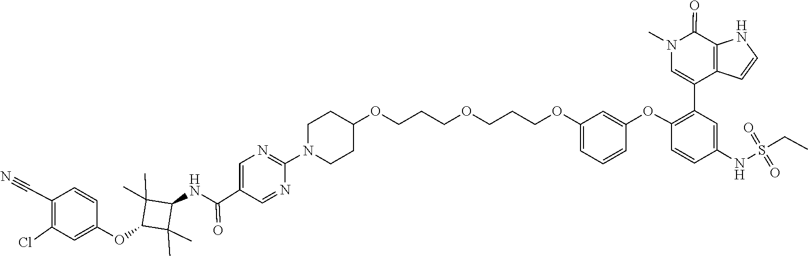 [SMILES: CCS(=O)(=O)Nc1ccc(Oc2cccc(OCCCOCCCOC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-13 | 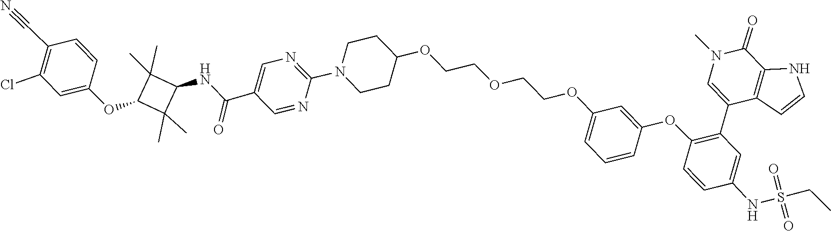 [SMILES: CCS(=O)(=O)Nc1ccc(Oc2cccc(OCCOCCOC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-14 | 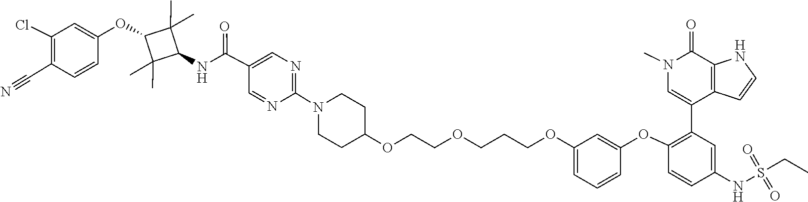 [SMILES: CCS(=O)(=O)Nc1ccc(Oc2cccc(OCCCOCCOC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-15 | 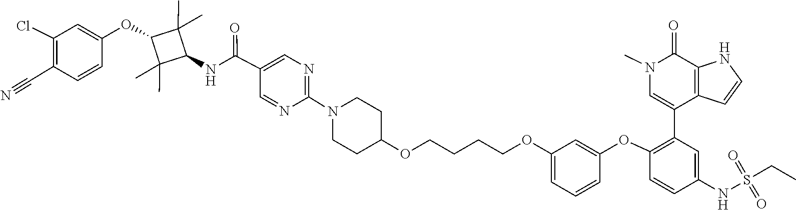 [SMILES: CCS(=O)(=O)Nc1ccc(Oc2cccc(OCCCCOC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-16 | 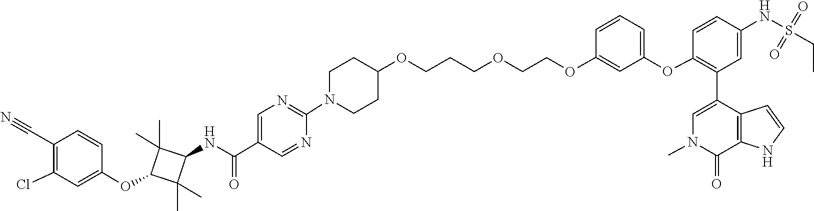 [SMILES: CCS(=O)(=O)Nc1ccc(Oc2cccc(OCCOCCCOC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-17 | 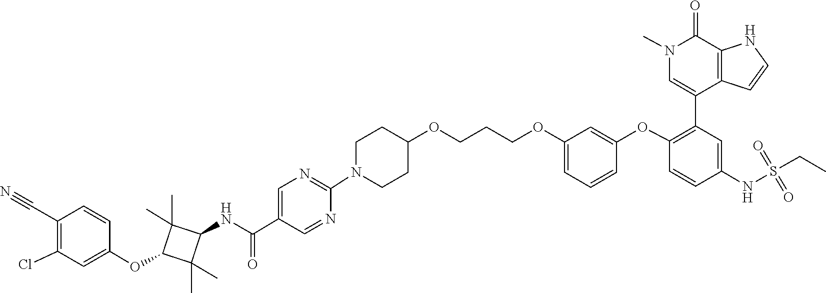 [SMILES: CCS(=O)(=O)Nc1ccc(Oc2cccc(OCCCOC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-18 | 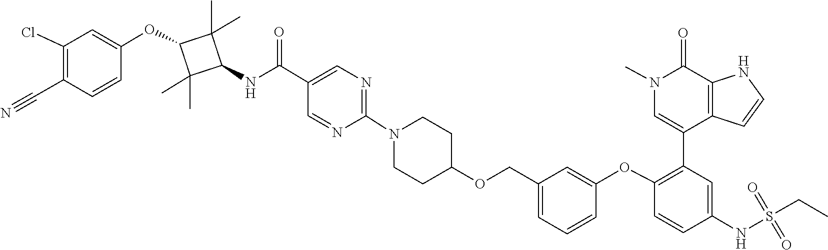 [SMILES: CCS(=O)(=O)Nc1ccc(Oc2cccc(COC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-19 | 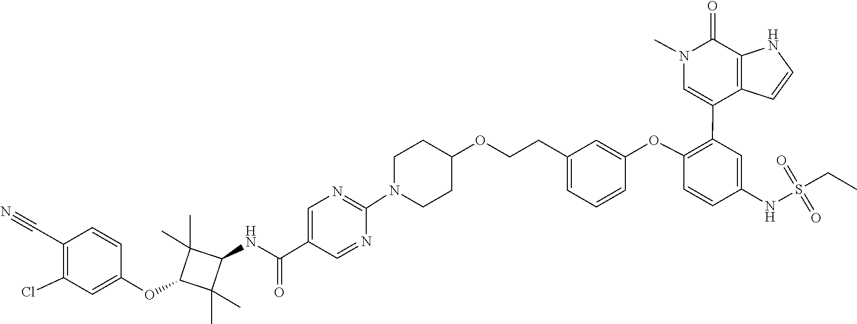 [SMILES: CCS(=O)(=O)Nc1ccc(Oc2cccc(CCOC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-20 | 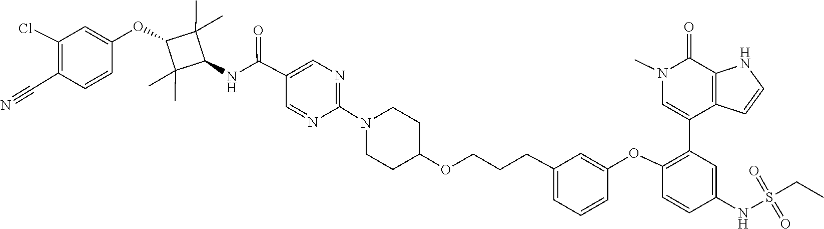 [SMILES: CCS(=O)(=O)Nc1ccc(Oc2cccc(CCCOC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-21 | 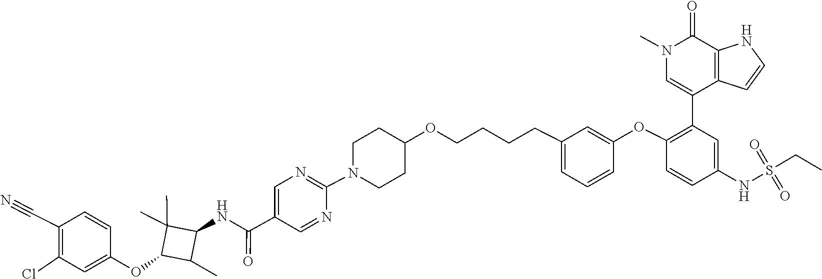 [SMILES: CCS(=O)(=O)Nc1ccc(Oc2cccc(CCCCOC3CCN(c4ncc(C(=O)N[C@H]5C(C)[C@H](Oc6ccc(C#N)c(Cl)c6)C5(C)C)cn4)CC3)c2)c(-c2cn(C)c(=O)c3[nH]ccc23)c1] |
| I-22 | 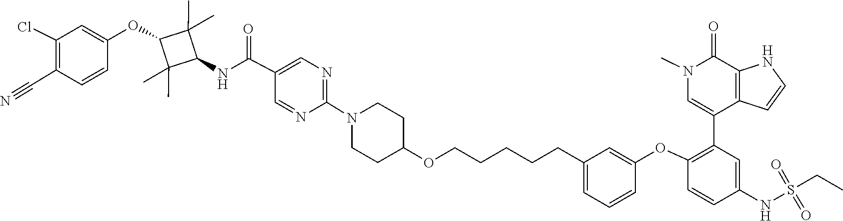 [SMILES: CCS(=O)(=O)Nc1ccc(Oc2cccc(CCCCCOC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-23 | 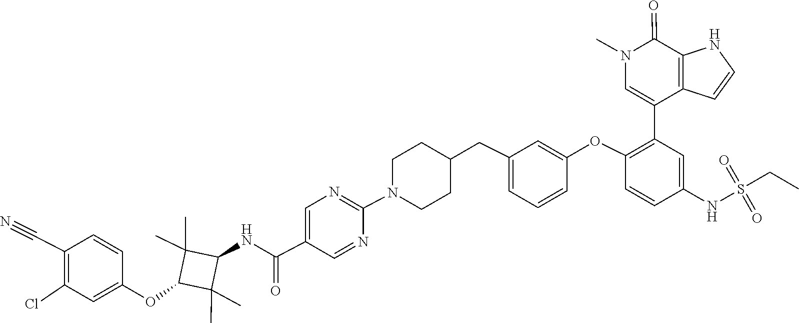 [SMILES: CCS(=O)(=O)Nc1ccc(Oc2cccc(CC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-24 | 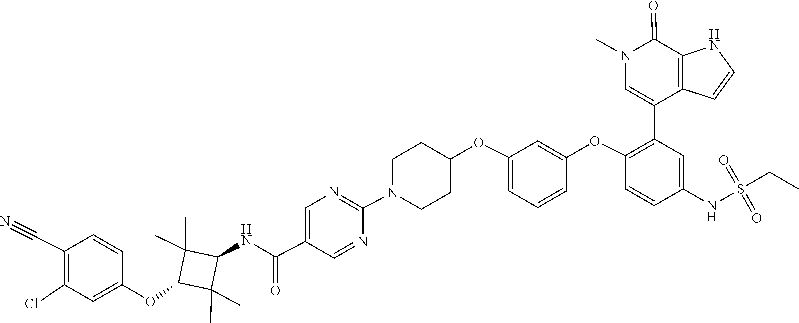 [SMILES: CCS(=O)(=O)Nc1ccc(Oc2cccc(OC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-25 | 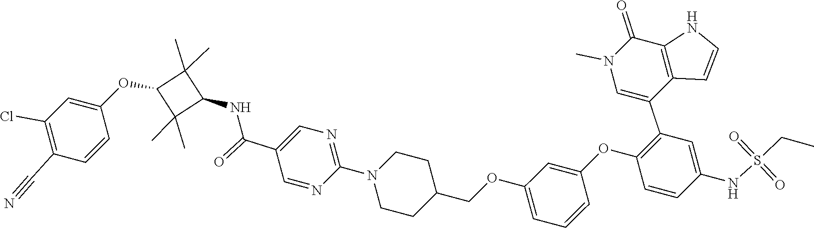 [SMILES: CCS(=O)(=O)Nc1ccc(Oc2cccc(OCC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-26 | 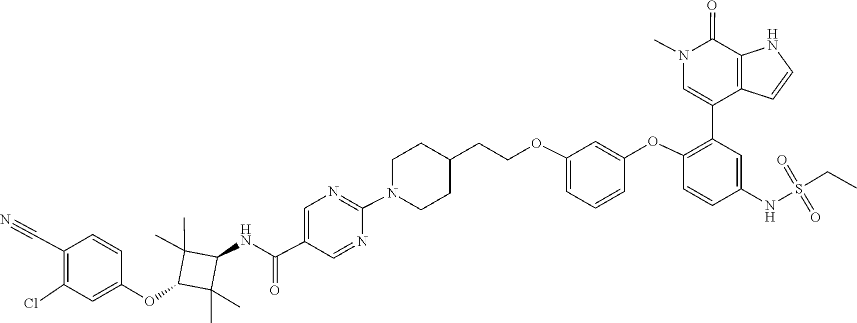 [SMILES: CCS(=O)(=O)Nc1ccc(Oc2cccc(OCCC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-27 | 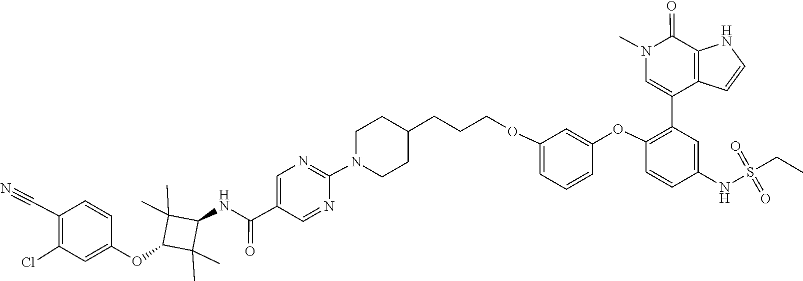 [SMILES: CCS(=O)(=O)Nc1ccc(Oc2cccc(OCCCC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-28 | 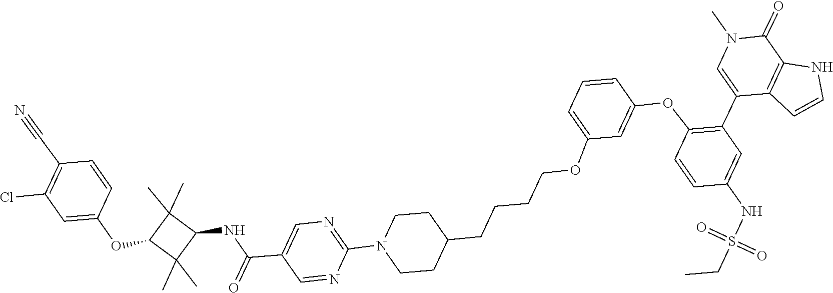 [SMILES: CCS(=O)(=O)Nc1ccc(Oc2cccc(OCCCCC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-29 | 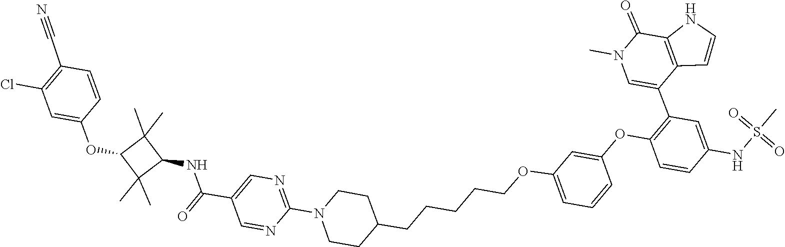 [SMILES: Cn1cc(-c2cc(NS(C)(=O)=O)ccc2Oc2cccc(OCCCCCC3CCN(c4ncc(C(=O)N[C@H]5C(C)(C)[C@H](Oc6ccc(C#N)c(Cl)c6)C5(C)C)cn4)CC3)c2)c2cc[nH]c2c1=O] |
| I-30 | 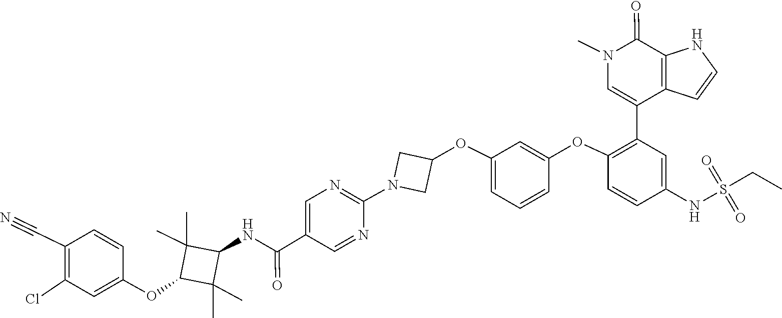 [SMILES: CCS(=O)(=O)Nc1ccc(Oc2cccc(OC3CN(c4ncc(C(=O)N[C@H]5C(C)(C)[C@H](Oc6ccc(C#N)c(Cl)c6)C5(C)C)cn4)C3)c2)c(-c2cn(C)c(=O)c3[nH]ccc23)c1] |
| I-31 | 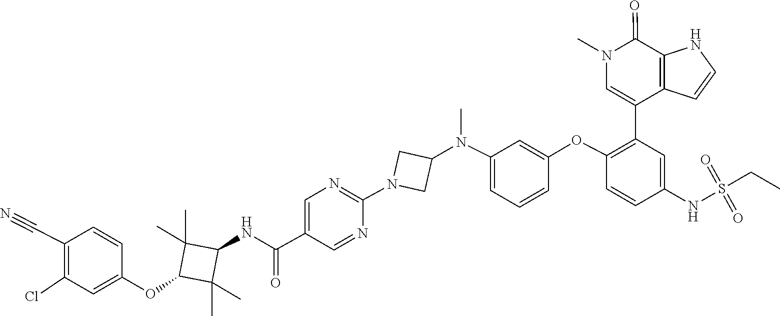 [SMILES: CCS(=O)(=O)Nc1ccc(Oc2cccc(N(C)C3CN(c4ncc(C(=O)N[C@H]5C(C)(C)[C@H](Oc6ccc(C#N)c(Cl)c6)C5(C)C)cn4)C3)c2)c(-c2cn(C)c(=O)c3[nH]ccc23)c1] |
| I-32 | 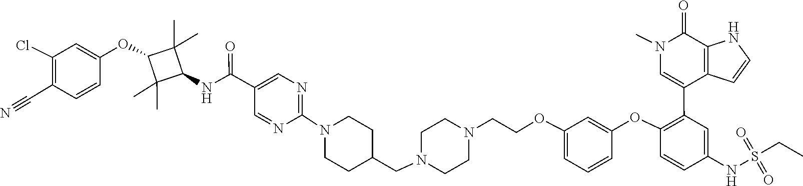 [SMILES: CCS(=O)(=O)Nc1ccc(Oc2cccc(OCCN3CCN(CC4CCN(c5ncc(C(=O)N[C@H]6C(C)(C)[C@H](Oc7ccc(C#N)c(Cl)c7)C6(C)C)cn5)CC4)CC3)c2)c(-c2cn(C)c(=O)c3[nH]ccc23)c1] |
| I-33 | 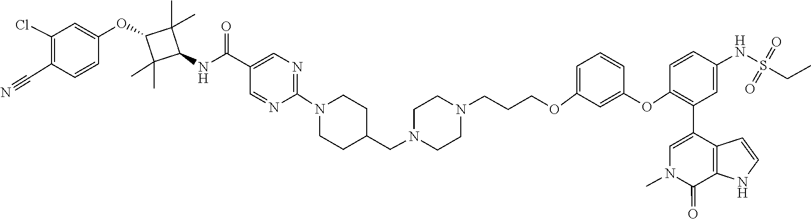 [SMILES: CCS(=O)(=O)Nc1ccc(Oc2cccc(OCCCN3CCN(CC4CCN(c5ncc(C(=O)N[C@H]6C(C)(C)[C@H](Oc7ccc(C#N)c(Cl)c7)C6(C)C)cn5)CC4)CC3)c2)c(-c2cn(C)c(=O)c3[nH]ccc23)c1] |
| I-34 | 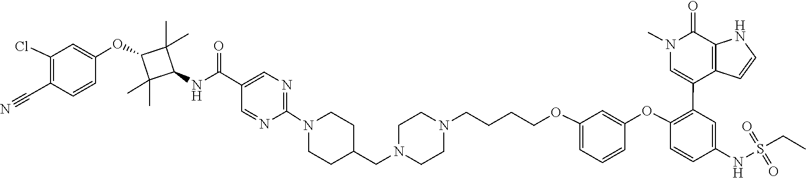 [SMILES: CCS(=O)(=O)Nc1ccc(Oc2cccc(OCCCCN3CCN(CC4CCN(c5ncc(C(=O)N[C@H]6C(C)(C)[C@H](Oc7ccc(C#N)c(Cl)c7)C6(C)C)cn5)CC4)CC3)c2)c(-c2cn(C)c(=O)c3[nH]ccc23)c1] |
| I-35 | 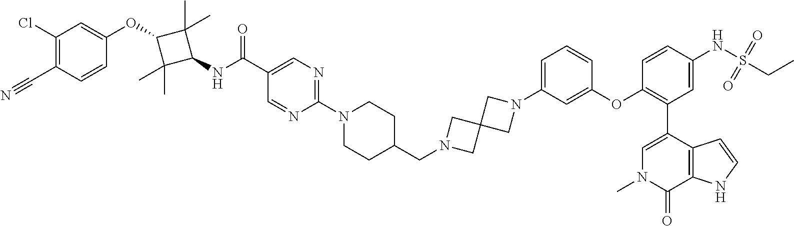 [SMILES: CCS(=O)(=O)Nc1ccc(Oc2cccc(N3CC4(CN(CC5CCN(c6ncc(C(=O)N[C@H]7C(C)(C)[C@H](Oc8ccc(C#N)c(Cl)c8)C7(C)C)cn6)CC5)C4)C3)c2)c(-c2cn(C)c(=O)c3[nH]ccc23)c1] |
| I-36 | 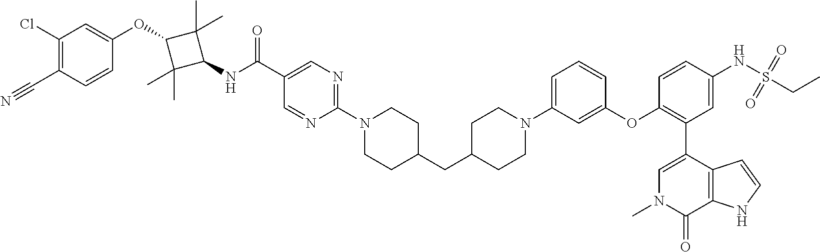 [SMILES: CCS(=O)(=O)Nc1ccc(Oc2cccc(N3CCC(CC4CCN(c5ncc(C(=O)N[C@H]6C(C)(C)[C@H](Oc7ccc(C#N)c(Cl)c7)C6(C)C)cn5)CC4)CC3)c2)c(-c2cn(C)c(=O)c3[nH]ccc23)c1] |
| I-37 | 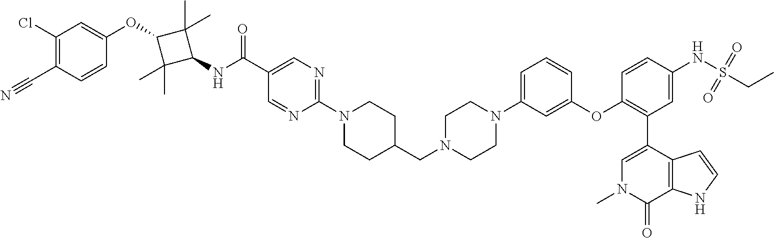 [SMILES: CCS(=O)(=O)Nc1ccc(Oc2cccc(N3CCN(CC4CCN(c5ncc(C(=O)N[C@H]6C(C)(C)[C@H](Oc7ccc(C#N)c(Cl)c7)C6(C)C)cn5)CC4)CC3)c2)c(-c2cn(C)c(=O)c3[nH]ccc23)c1] |
| I-38 | 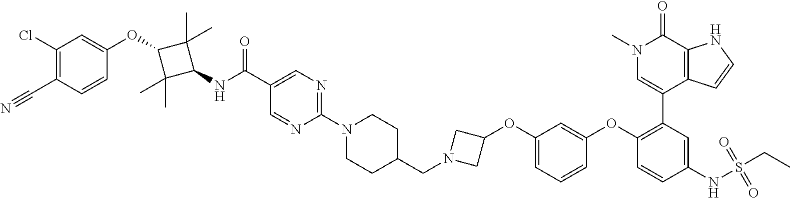 [SMILES: CCS(=O)(=O)Nc1ccc(Oc2cccc(OC3CN(CC4CCN(c5ncc(C(=O)N[C@H]6C(C)(C)[C@H](Oc7ccc(C#N)c(Cl)c7)C6(C)C)cn5)CC4)C3)c2)c(-c2cn(C)c(=O)c3[nH]ccc23)c1] |
| I-39 | 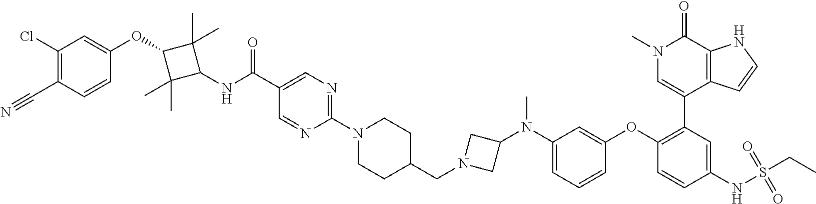 |
| I-40 | 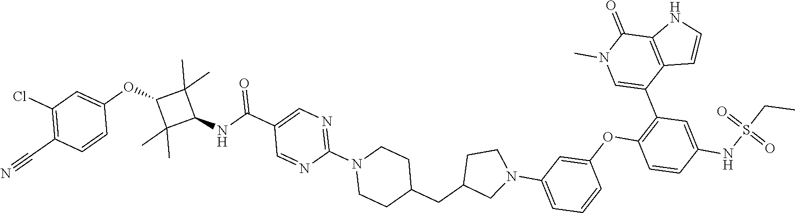 [SMILES: CCS(=O)(=O)Nc1ccc(Oc2cccc(N3CCC(CC4CCN(c5ncc(C(=O)N[C@H]6C(C)(C)[C@H](Oc7ccc(C#N)c(Cl)c7)C6(C)C)cn5)CC4)C3)c2)c(-c2cn(C)c(=O)c3[nH]ccc23)c1] |
| I-41 | 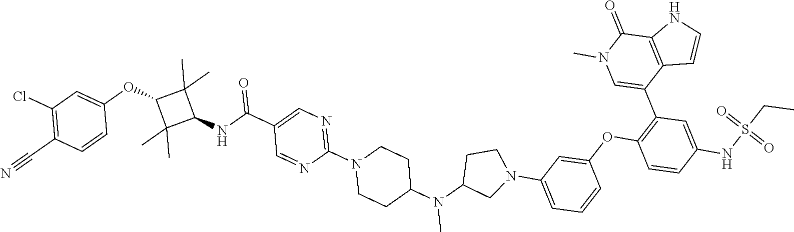 [SMILES: CCS(=O)(=O)Nc1ccc(Oc2cccc(N3CCC(N(C)C4CCN(c5ncc(C(=O)N[C@H]6C(C)(C)[C@H](Oc7ccc(C#N)c(Cl)c7)C6(C)C)cn5)CC4)C3)c2)c(-c2cn(C)c(=O)c3[nH]ccc23)c1] |
| I-42 | 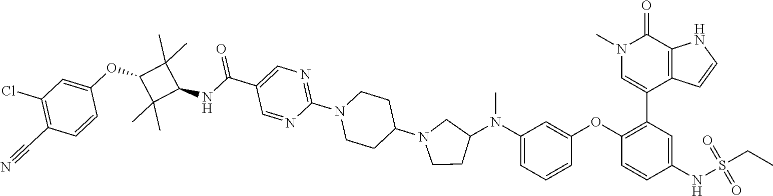 [SMILES: CCS(=O)(=O)Nc1ccc(Oc2cccc(N(C)C3CCN(C4CCN(c5ncc(C(=O)N[C@H]6C(C)(C)[C@H](Oc7ccc(C#N)c(Cl)c7)C6(C)C)cn5)CC4)C3)c2)c(-c2cn(C)c(=O)c3[nH]ccc23)c1] |
| I-43 | 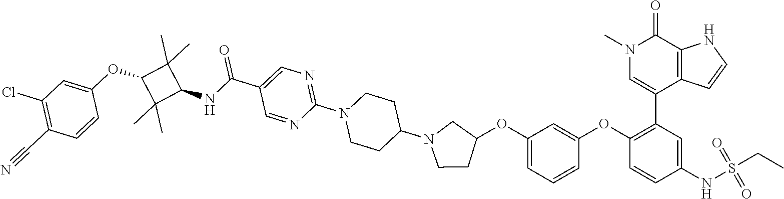 [SMILES: CCS(=O)(=O)Nc1ccc(Oc2cccc(OC3CCN(C4CCN(c5ncc(C(=O)N[C@H]6C(C)(C)[C@H](Oc7ccc(C#N)c(Cl)c7)C6(C)C)cn5)CC4)C3)c2)c(-c2cn(C)c(=O)c3[nH]ccc23)c1] |
| I-44 | 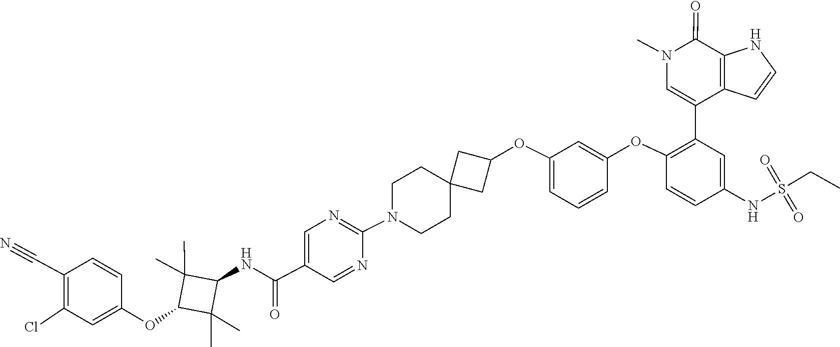 [SMILES: CCS(=O)(=O)Nc1ccc(Oc2cccc(OC3CC4(CCN(c5ncc(C(=O)N[C@H]6C(C)(C)[C@H](Oc7ccc(C#N)c(Cl)c7)C6(C)C)cn5)CC4)C3)c2)c(-c2cn(C)c(=O)c3[nH]ccc23)c1] |
| I-45 | 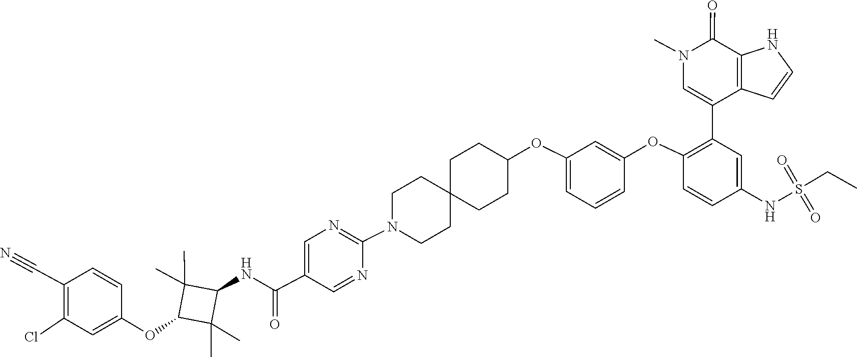 [SMILES: CCS(=O)(=O)Nc1ccc(Oc2cccc(OC3CCC4(CC3)CCN(c3ncc(C(=O)N[C@H]5C(C)(C)[C@H](Oc6ccc(C#N)c(Cl)c6)C5(C)C)cn3)CC4)c2)c(-c2cn(C)c(=O)c3[nH]ccc23)c1] |
| I-46 | 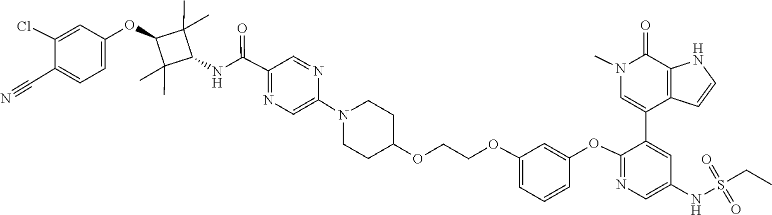 [SMILES: CCS(=O)(=O)Nc1cnc(Oc2cccc(OCCOC3CCN(c4cnc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-47 | 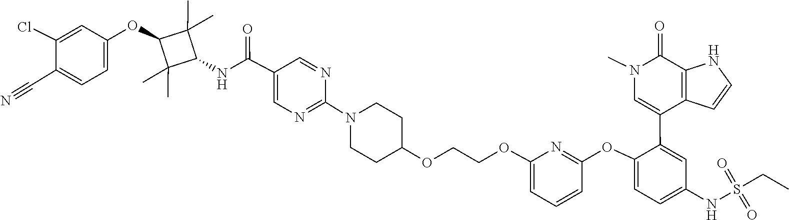 [SMILES: CCS(=O)(=O)Nc1ccc(Oc2cccc(OCCOC3CCN(c4ncc(C(=O)N[C@H]5C(C)(C)[C@H](Oc6ccc(C#N)c(Cl)c6)C5(C)C)cn4)CC3)n2)c(-c2cn(C)c(=O)c3[nH]ccc23)c1] |
| I-48 | 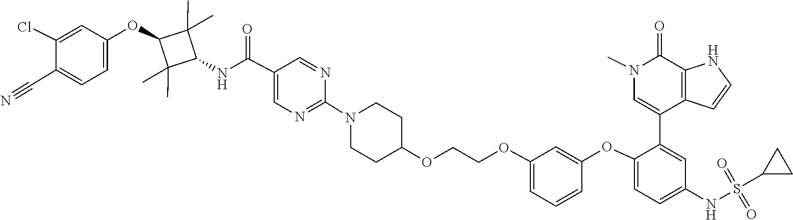 [SMILES: Cn1cc(-c2cc(NS(=O)(=O)C3CC3)ccc2Oc2cccc(OCCOC3CCN(c4ncc(C(=O)N[C@H]5C(C)(C)[C@H](Oc6ccc(C#N)c(Cl)c6)C5(C)C)cn4)CC3)c2)c2cc[nH]c2c1=O] |
| I-49 | 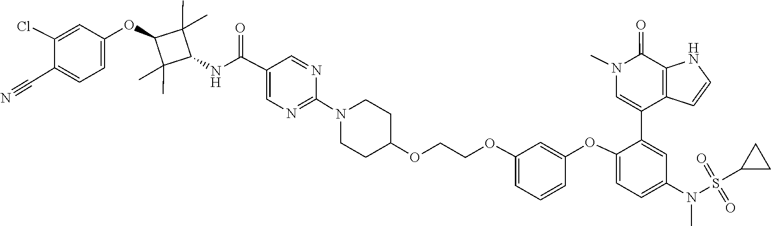 [SMILES: CN(c1ccc(Oc2cccc(OCCOC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1)S(=O)(=O)C1CC1] |
| I-50 | 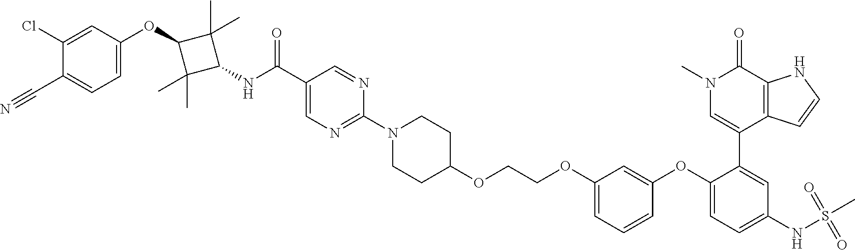 [SMILES: Cn1cc(-c2cc(NS(C)(=O)=O)ccc2Oc2cccc(OCCOC3CCN(c4ncc(C(=O)N[C@H]5C(C)(C)[C@H](Oc6ccc(C#N)c(Cl)c6)C5(C)C)cn4)CC3)c2)c2cc[nH]c2c1=O] |
| I-51 | 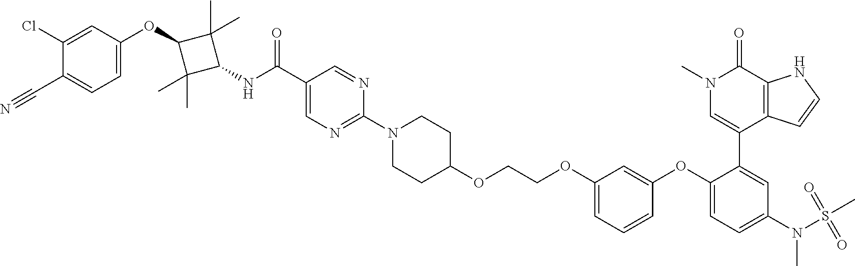 [SMILES: CN(c1ccc(Oc2cccc(OCCOC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1)S(C)(=O)=O] |
| I-52 | 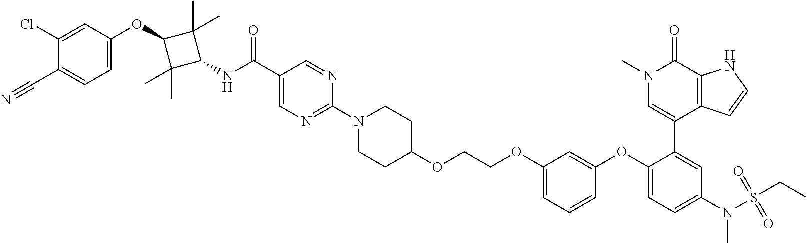 [SMILES: CCS(=O)(=O)N(C)c1ccc(Oc2cccc(OCCOC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-53 | 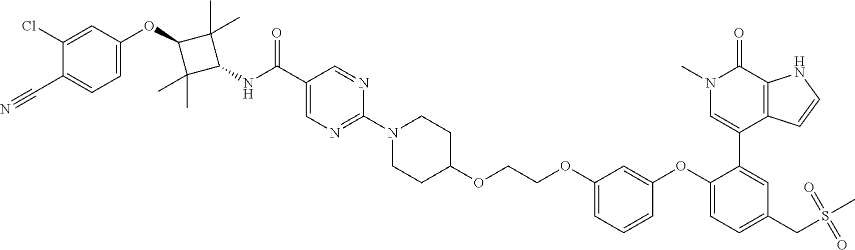 [SMILES: Cn1cc(-c2cc(CS(C)(=O)=O)ccc2Oc2cccc(OCCOC3CCN(c4ncc(C(=O)N[C@H]5C(C)(C)[C@H](Oc6ccc(C#N)c(Cl)c6)C5(C)C)cn4)CC3)c2)c2cc[nH]c2c1=O] |
| I-54 | 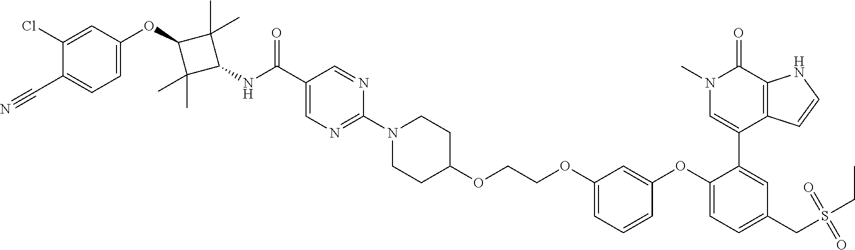 [SMILES: CCS(=O)(=O)Cc1ccc(Oc2cccc(OCCOC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-55 | 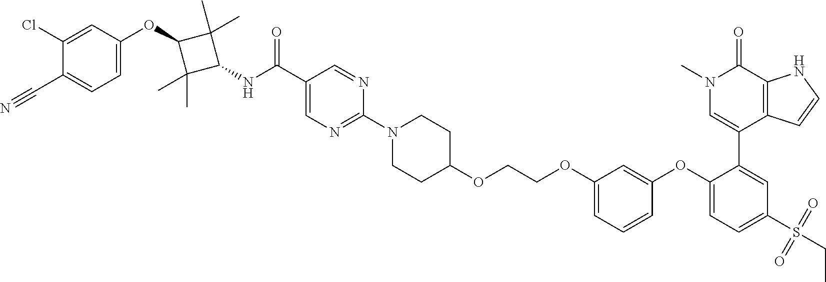 [SMILES: CCS(=O)(=O)c1ccc(Oc2cccc(OCCOC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-56 | 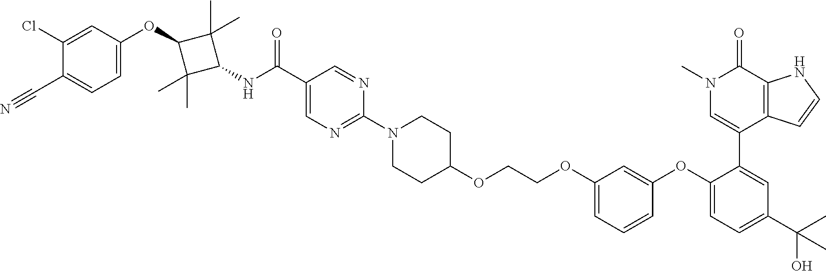 [SMILES: Cn1cc(-c2cc(C(C)(C)O)ccc2Oc2cccc(OCCOC3CCN(c4ncc(C(=O)N[C@H]5C(C)(C)[C@H](Oc6ccc(C#N)c(Cl)c6)C5(C)C)cn4)CC3)c2)c2cc[nH]c2c1=O] |
| I-57 | 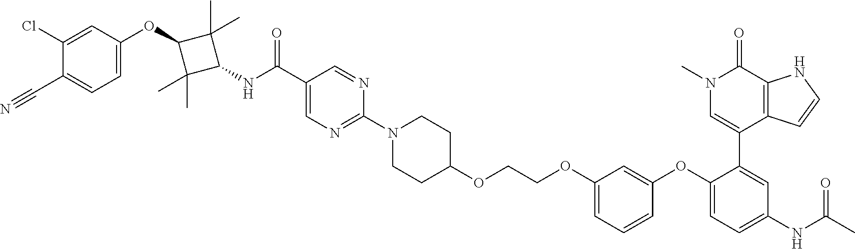 [SMILES: CC(=O)Nc1ccc(Oc2cccc(OCCOC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-58 | 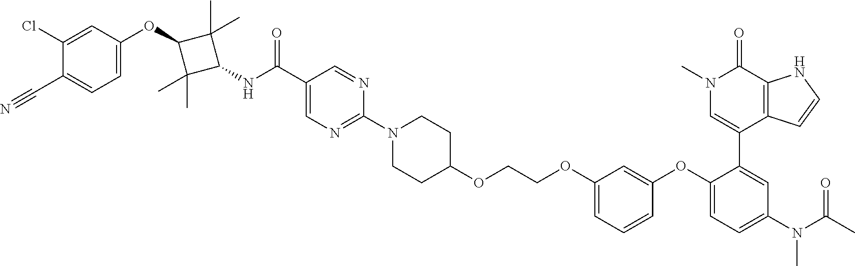 [SMILES: CC(=O)N(C)c1ccc(Oc2cccc(OCCOC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-59 | 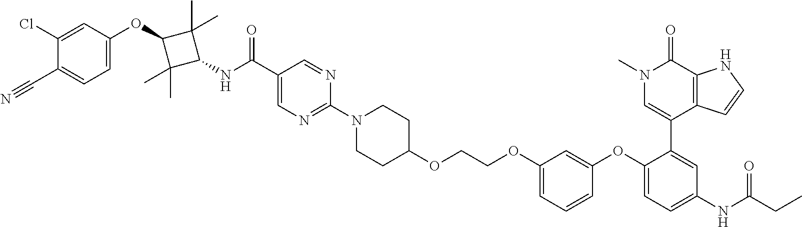 [SMILES: CCC(=O)Nc1ccc(Oc2cccc(OCCOC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-60 | 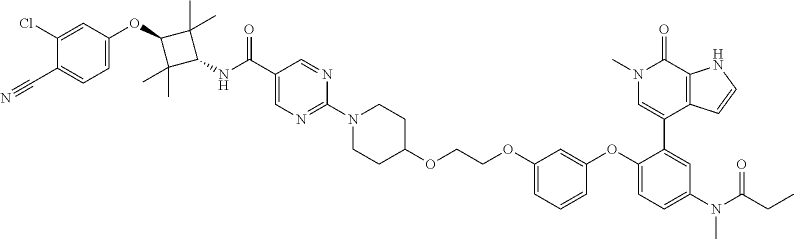 [SMILES: CCC(=O)N(C)c1ccc(Oc2cccc(OCCOC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-61 | 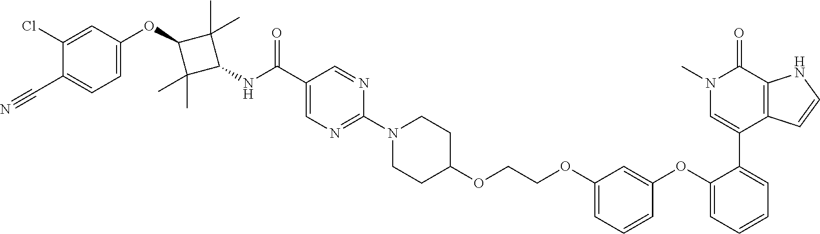 [SMILES: Cn1cc(-c2ccccc2Oc2cccc(OCCOC3CCN(c4ncc(C(=O)N[C@H]5C(C)(C)[C@H](Oc6ccc(C#N)c(Cl)c6)C5(C)C)cn4)CC3)c2)c2cc[nH]c2c1=O] |
| I-62 | 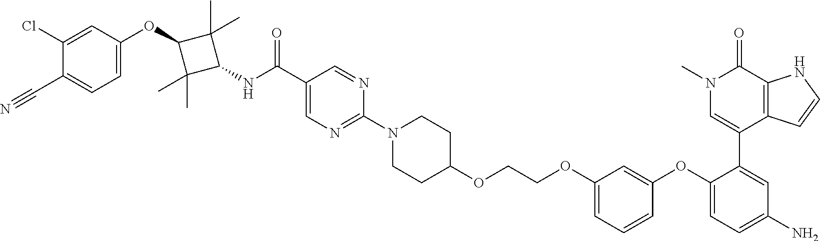 [SMILES: Cn1cc(-c2cc(N)ccc2Oc2cccc(OCCOC3CCN(c4ncc(C(=O)N[C@H]5C(C)(C)[C@H](Oc6ccc(C#N)c(Cl)c6)C5(C)C)cn4)CC3)c2)c2cc[nH]c2c1=O] |
| I-63 | 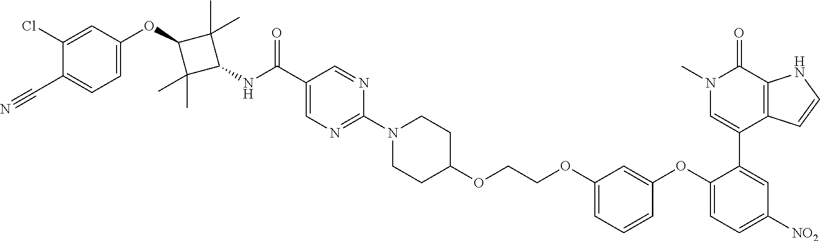 [SMILES: Cn1cc(-c2cc([N+](=O)[O-])ccc2Oc2cccc(OCCOC3CCN(c4ncc(C(=O)N[C@H]5C(C)(C)[C@H](Oc6ccc(C#N)c(Cl)c6)C5(C)C)cn4)CC3)c2)c2cc[nH]c2c1=O] |
| I-64 | 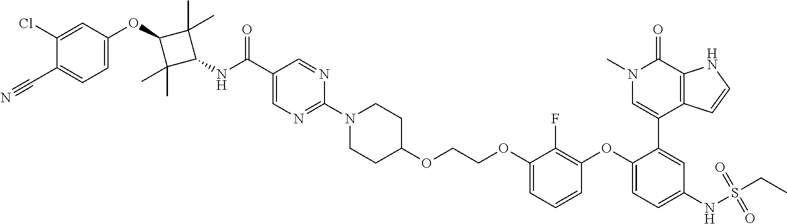 [SMILES: CCS(=O)(=O)Nc1ccc(Oc2cccc(OCCOC3CCN(c4ncc(C(=O)N[C@H]5C(C)(C)[C@H](Oc6ccc(C#N)c(Cl)c6)C5(C)C)cn4)CC3)c2F)c(-c2cn(C)c(=O)c3[nH]ccc23)c1] |
| I-65 | 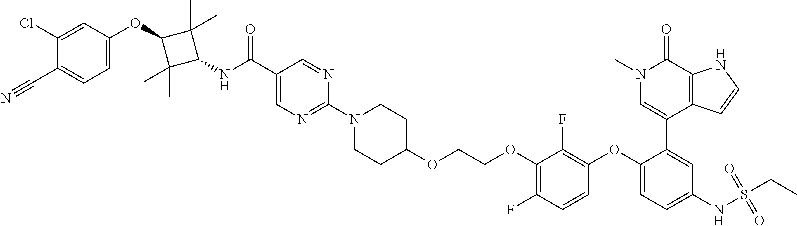 [SMILES: CCS(=O)(=O)Nc1ccc(Oc2ccc(F)c(OCCOC3CCN(c4ncc(C(=O)N[C@H]5C(C)(C)[C@H](Oc6ccc(C#N)c(Cl)c6)C5(C)C)cn4)CC3)c2F)c(-c2cn(C)c(=O)c3[nH]ccc23)c1] |
| I-66 | 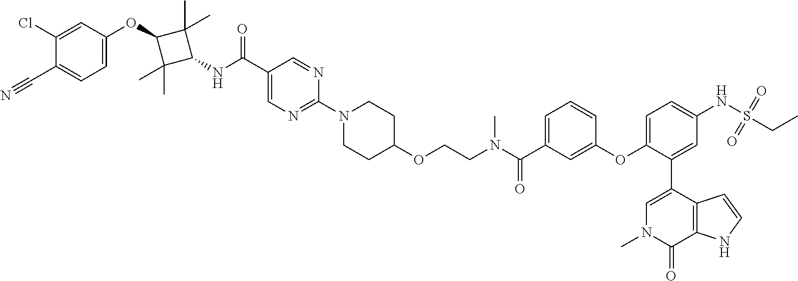 [SMILES: CCS(=O)(=O)Nc1ccc(Oc2cccc(C(=O)N(C)CCOC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-67 | 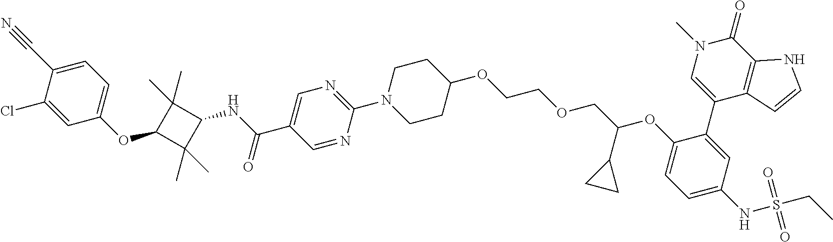 [SMILES: CCS(=O)(=O)Nc1ccc(OC(COCCOC2CCN(c3ncc(C(=O)N[C@H]4C(C)(C)[C@H](Oc5ccc(C#N)c(Cl)c5)C4(C)C)cn3)CC2)C2CC2)c(-c2cn(C)c(=O)c3[nH]ccc23)c1] |
| I-68 | 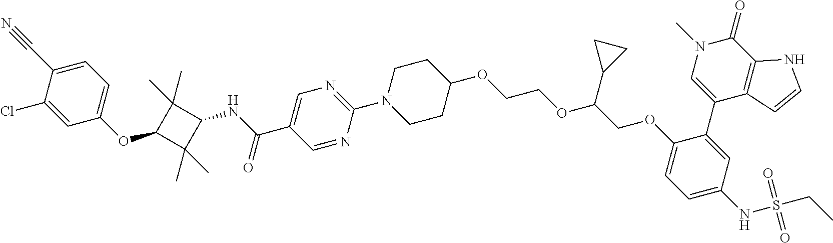 [SMILES: CCS(=O)(=O)Nc1ccc(OCC(OCCOC2CCN(c3ncc(C(=O)N[C@H]4C(C)(C)[C@H](Oc5ccc(C#N)c(Cl)c5)C4(C)C)cn3)CC2)C2CC2)c(-c2cn(C)c(=O)c3[nH]ccc23)c1] |
| I-69 | 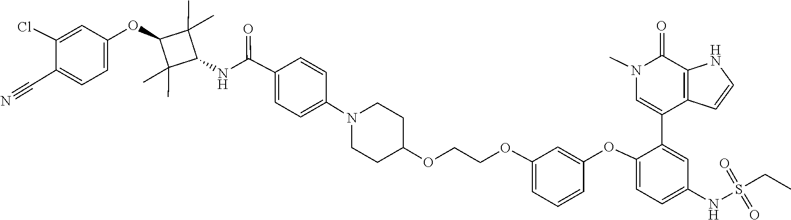 [SMILES: CCS(=O)(=O)Nc1ccc(Oc2cccc(OCCOC3CCN(c4ccc(C(=O)N[C@H]5C(C)(C)[C@H](Oc6ccc(C#N)c(Cl)c6)C5(C)C)cc4)CC3)c2)c(-c2cn(C)c(=O)c3[nH]ccc23)c1] |
| I-70 | 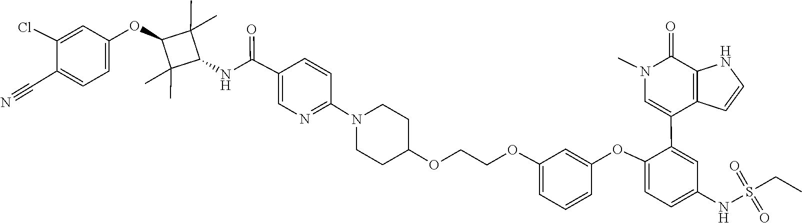 [SMILES: CCS(=O)(=O)Nc1ccc(Oc2cccc(OCCOC3CCN(c4ccc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-71 | 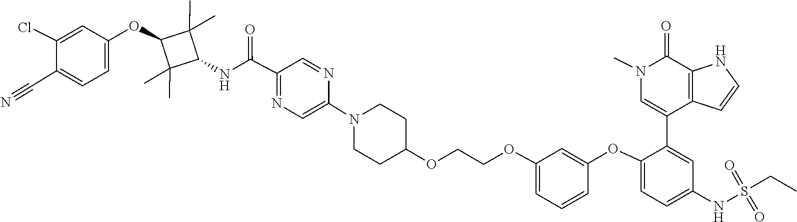 [SMILES: CCS(=O)(=O)Nc1ccc(Oc2cccc(OCCOC3CCN(c4cnc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-72 | 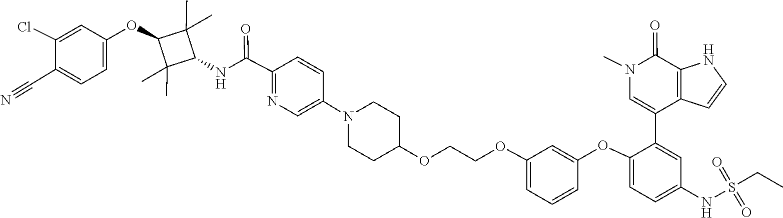 [SMILES: CCS(=O)(=O)Nc1ccc(Oc2cccc(OCCOC3CCN(c4ccc(C(=O)N[C@H]5C(C)(C)[C@H](Oc6ccc(C#N)c(Cl)c6)C5(C)C)nc4)CC3)c2)c(-c2cn(C)c(=O)c3[nH]ccc23)c1] |
| I-73 | 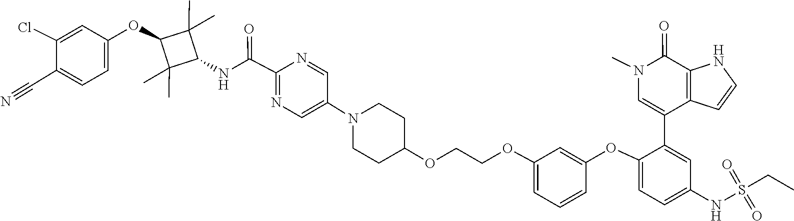 [SMILES: CCS(=O)(=O)Nc1ccc(Oc2cccc(OCCOC3CCN(c4cnc(C(=O)N[C@H]5C(C)(C)[C@H](Oc6ccc(C#N)c(Cl)c6)C5(C)C)nc4)CC3)c2)c(-c2cn(C)c(=O)c3[nH]ccc23)c1] |
| I-74 | 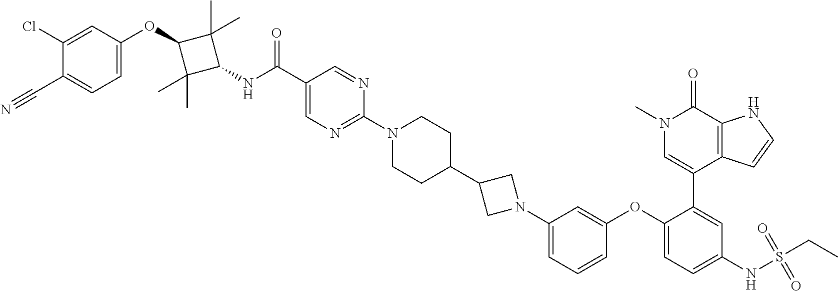 [SMILES: CCS(=O)(=O)Nc1ccc(Oc2cccc(N3CC(C4CCN(c5ncc(C(=O)N[C@H]6C(C)(C)[C@H](Oc7ccc(C#N)c(Cl)c7)C6(C)C)cn5)CC4)C3)c2)c(-c2cn(C)c(=O)c3[nH]ccc23)c1] |
| I-75 | 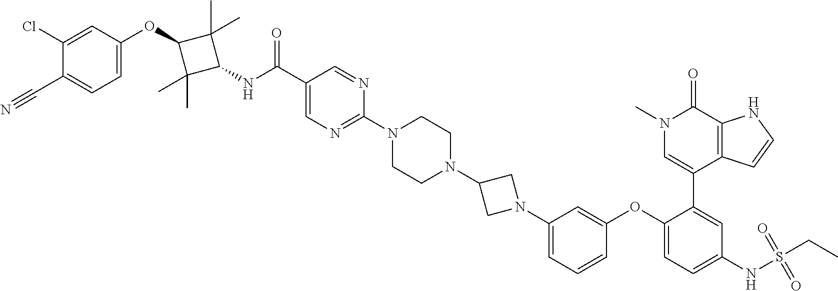 [SMILES: CCS(=O)(=O)Nc1ccc(Oc2cccc(N3CC(N4CCN(c5ncc(C(=O)N[C@H]6C(C)(C)[C@H](Oc7ccc(C#N)c(Cl)c7)C6(C)C)cn5)CC4)C3)c2)c(-c2cn(C)c(=O)c3[nH]ccc23)c1] |
| I-76 | 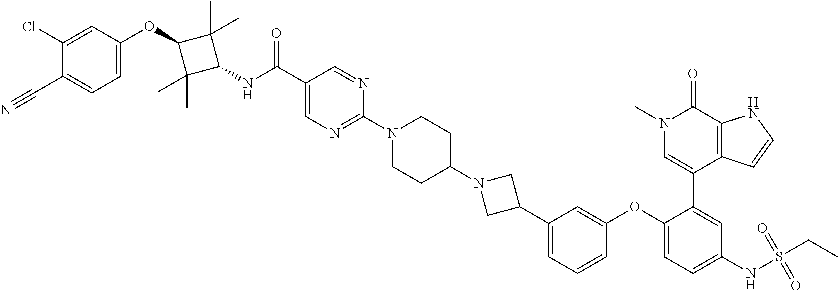 [SMILES: CCS(=O)(=O)Nc1ccc(Oc2cccc(C3CN(C4CCN(c5ncc(C(=O)N[C@H]6C(C)(C)[C@H](Oc7ccc(C#N)c(Cl)c7)C6(C)C)cn5)CC4)C3)c2)c(-c2cn(C)c(=O)c3[nH]ccc23)c1] |
| I-77 | 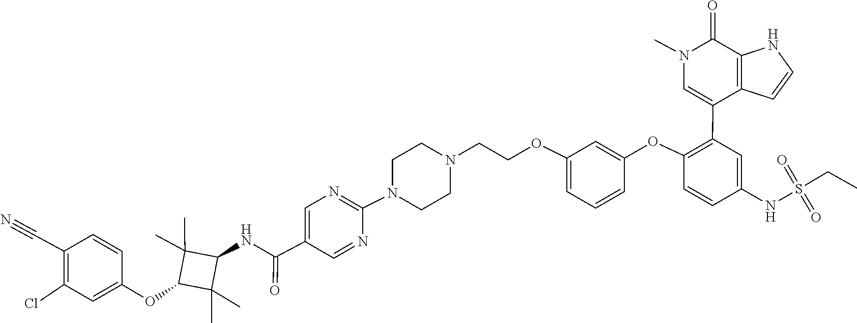 [SMILES: CCS(=O)(=O)Nc1ccc(Oc2cccc(OCCN3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-78 | 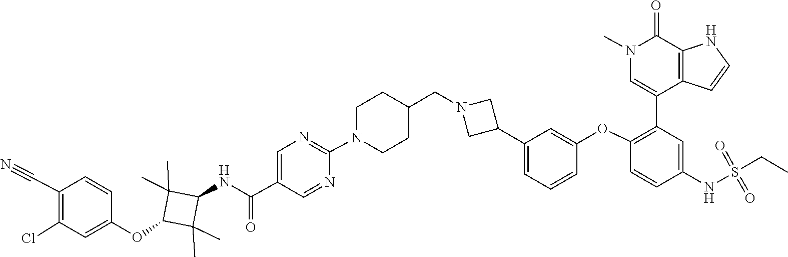 [SMILES: CCS(=O)(=O)Nc1ccc(Oc2cccc(C3CN(CC4CCN(c5ncc(C(=O)N[C@H]6C(C)(C)[C@H](Oc7ccc(C#N)c(Cl)c7)C6(C)C)cn5)CC4)C3)c2)c(-c2cn(C)c(=O)c3[nH]ccc23)c1] |
| I-79 | 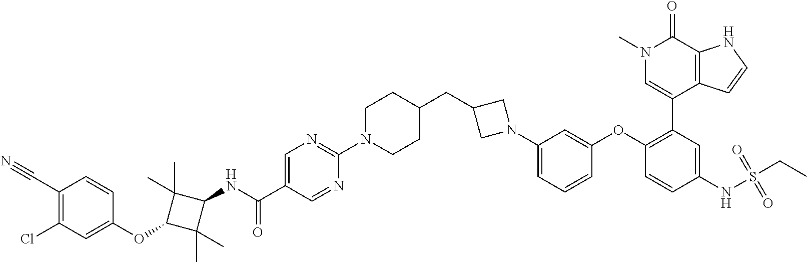 [SMILES: CCS(=O)(=O)Nc1ccc(Oc2cccc(N3CC(CC4CCN(c5ncc(C(=O)N[C@H]6C(C)(C)[C@H](Oc7ccc(C#N)c(Cl)c7)C6(C)C)cn5)CC4)C3)c2)c(-c2cn(C)c(=O)c3[nH]ccc23)c1] |
| I-80 | 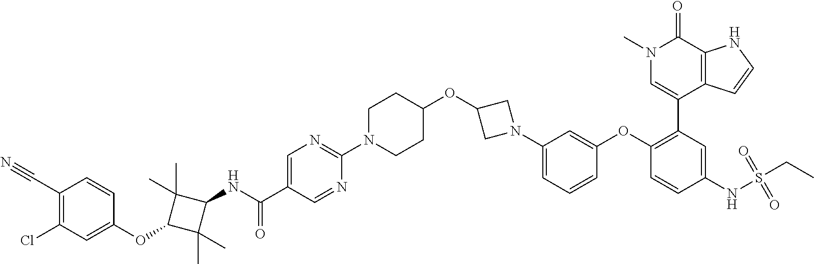 [SMILES: CCS(=O)(=O)Nc1ccc(Oc2cccc(N3CC(OC4CCN(c5ncc(C(=O)N[C@H]6C(C)(C)[C@H](Oc7ccc(C#N)c(Cl)c7)C6(C)C)cn5)CC4)C3)c2)c(-c2cn(C)c(=O)c3[nH]ccc23)c1] |
| I-81 | 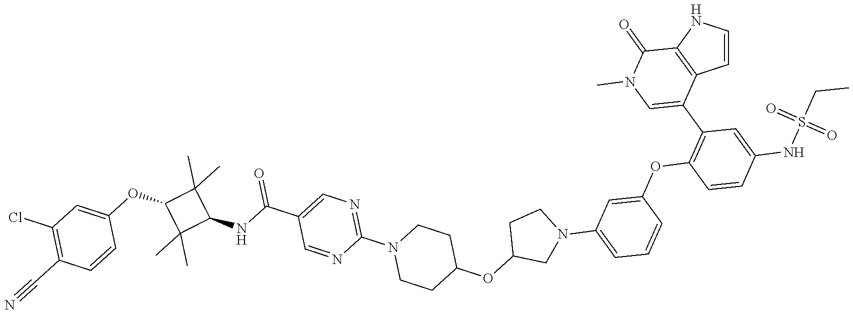 [SMILES: CCS(=O)(=O)Nc1ccc(Oc2cccc(N3CCC(OC4CCN(c5ncc(C(=O)N[C@H]6C(C)(C)[C@H](Oc7ccc(C#N)c(Cl)c7)C6(C)C)cn5)CC4)C3)c2)c(-c2cn(C)c(=O)c3[nH]ccc23)c1] |
| I-82 | 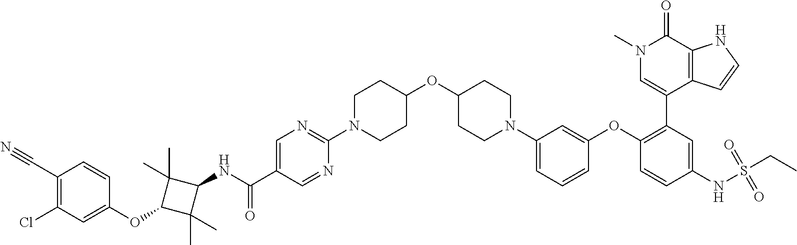 [SMILES: CCS(=O)(=O)Nc1ccc(Oc2cccc(N3CCC(OC4CCN(c5ncc(C(=O)N[C@H]6C(C)(C)[C@H](Oc7ccc(C#N)c(Cl)c7)C6(C)C)cn5)CC4)CC3)c2)c(-c2cn(C)c(=O)c3[nH]ccc23)c1] |
| I-83 | 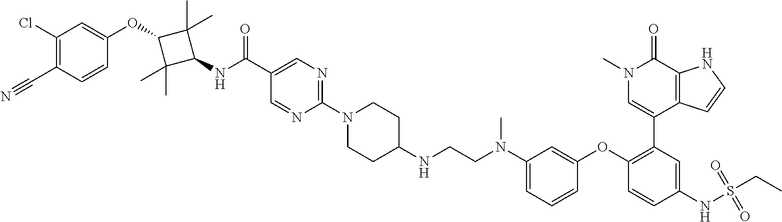 [SMILES: CCS(=O)(=O)Nc1ccc(Oc2cccc(N(C)CCNC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-84 | 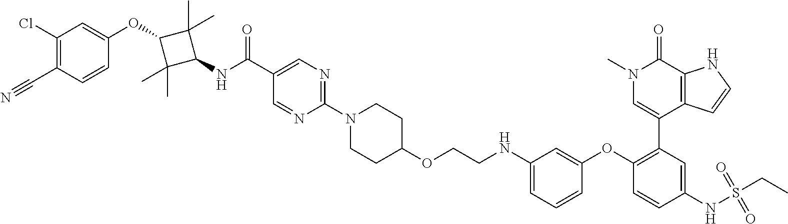 [SMILES: CCS(=O)(=O)Nc1ccc(Oc2cccc(NCCOC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-85 | 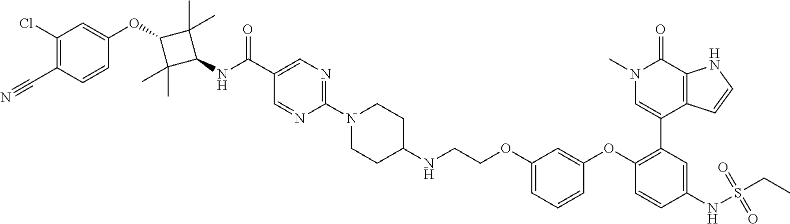 [SMILES: CCS(=O)(=O)Nc1ccc(Oc2cccc(OCCNC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-86 | 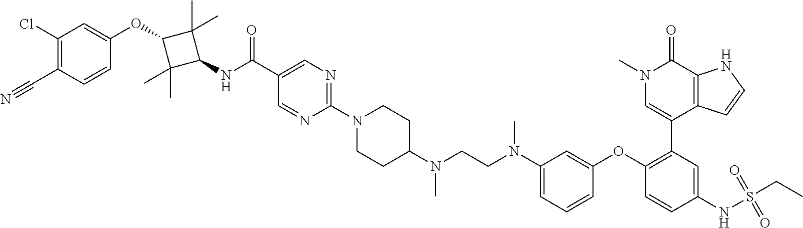 [SMILES: CCS(=O)(=O)Nc1ccc(Oc2cccc(N(C)CCN(C)C3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-87 | 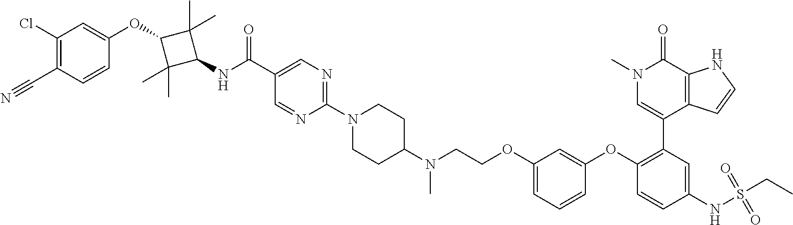 [SMILES: CCS(=O)(=O)Nc1ccc(Oc2cccc(OCCN(C)C3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-88 | 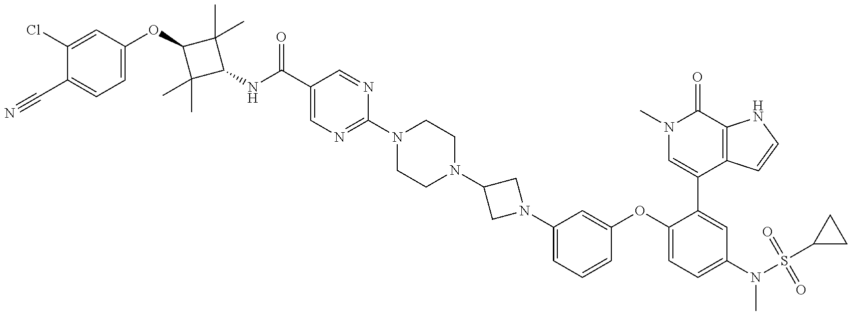 [SMILES: CN(c1ccc(Oc2cccc(N3CC(N4CCN(c5ncc(C(=O)N[C@H]6C(C)(C)[C@H](Oc7ccc(C#N)c(Cl)c7)C6(C)C)cn5)CC4)C3)c2)c(-c2cn(C)c(=O)c3[nH]ccc23)c1)S(=O)(=O)C1CC1] |
| I-89 | 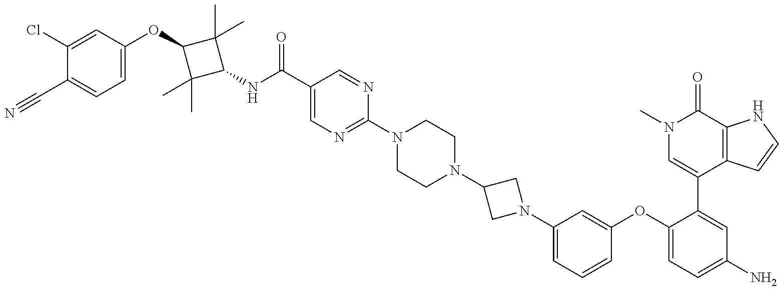 [SMILES: Cn1cc(-c2cc(N)ccc2Oc2cccc(N3CC(N4CCN(c5ncc(C(=O)N[C@H]6C(C)(C)[C@H](Oc7ccc(C#N)c(Cl)c7)C6(C)C)cn5)CC4)C3)c2)c2cc[nH]c2c1=O] |
| I-90 | 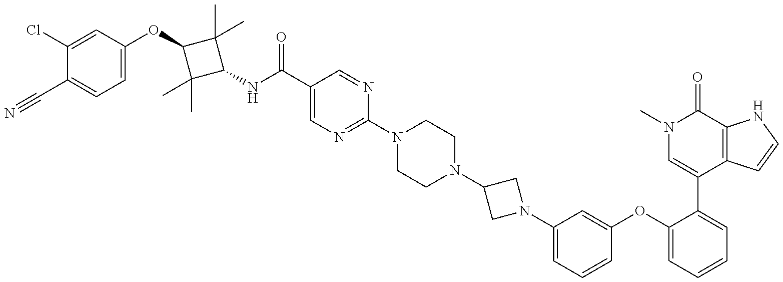 [SMILES: Cn1cc(-c2ccccc2Oc2cccc(N3CC(N4CCN(c5ncc(C(=O)N[C@H]6C(C)(C)[C@H](Oc7ccc(C#N)c(Cl)c7)C6(C)C)cn5)CC4)C3)c2)c2cc[nH]c2c1=O] |
| I-91 | 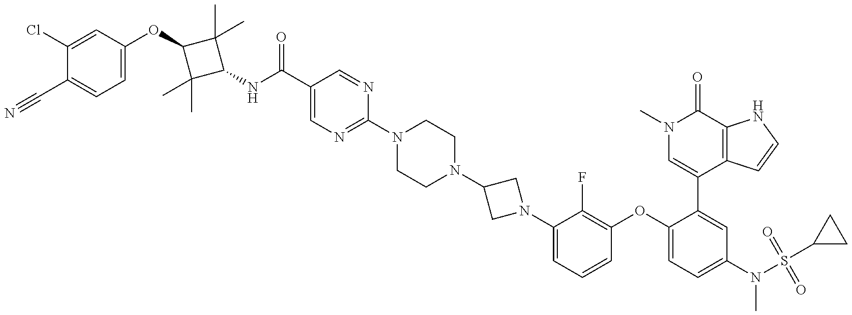 [SMILES: CN(c1ccc(Oc2cccc(N3CC(N4CCN(c5ncc(C(=O)N[C@H]6C(C)(C)[C@H](Oc7ccc(C#N)c(Cl)c7)C6(C)C)cn5)CC4)C3)c2F)c(-c2cn(C)c(=O)c3[nH]ccc23)c1)S(=O)(=O)C1CC1] |
| I-92 | 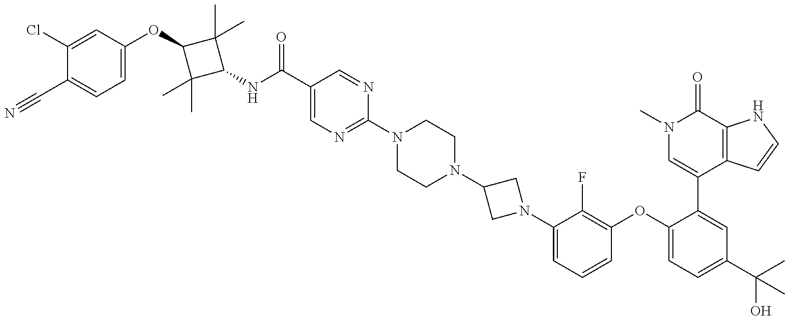 [SMILES: Cn1cc(-c2cc(C(C)(C)O)ccc2Oc2cccc(N3CC(N4CCN(c5ncc(C(=O)N[C@H]6C(C)(C)[C@H](Oc7ccc(C#N)c(Cl)c7)C6(C)C)cn5)CC4)C3)c2F)c2cc[nH]c2c1=O] |
| I-93 | 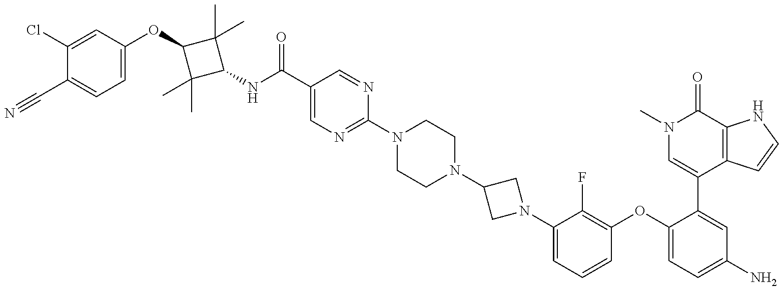 [SMILES: Cn1cc(-c2cc(N)ccc2Oc2cccc(N3CC(N4CCN(c5ncc(C(=O)N[C@H]6C(C)(C)[C@H](Oc7ccc(C#N)c(Cl)c7)C6(C)C)cn5)CC4)C3)c2F)c2cc[nH]c2c1=O] |
| I-94 | 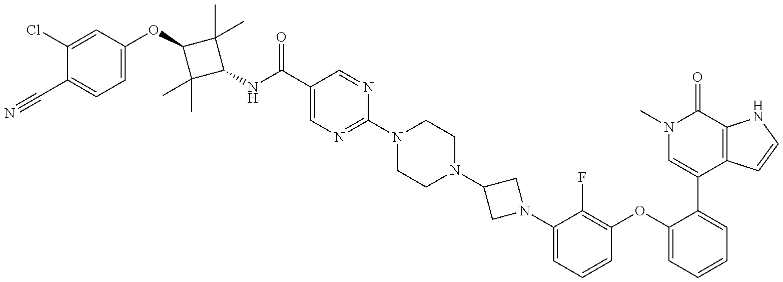 [SMILES: Cn1cc(-c2ccccc2Oc2cccc(N3CC(N4CCN(c5ncc(C(=O)N[C@H]6C(C)(C)[C@H](Oc7ccc(C#N)c(Cl)c7)C6(C)C)cn5)CC4)C3)c2F)c2cc[nH]c2c1=O] |
| I-95 | 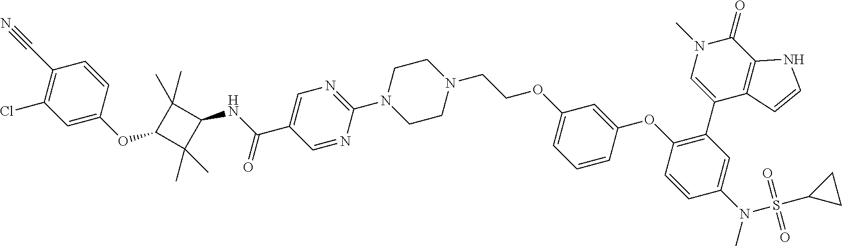 [SMILES: CN(c1ccc(Oc2cccc(OCCN3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1)S(=O)(=O)C1CC1] |
| I-96 | 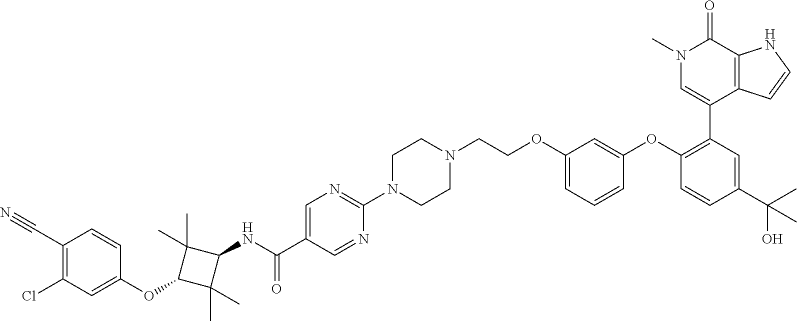 [SMILES: Cn1cc(-c2cc(C(C)(C)O)ccc2Oc2cccc(OCCN3CCN(c4ncc(C(=O)N[C@H]5C(C)(C)[C@H](Oc6ccc(C#N)c(Cl)c6)C5(C)C)cn4)CC3)c2)c2cc[nH]c2c1=O] |
| I-97 | 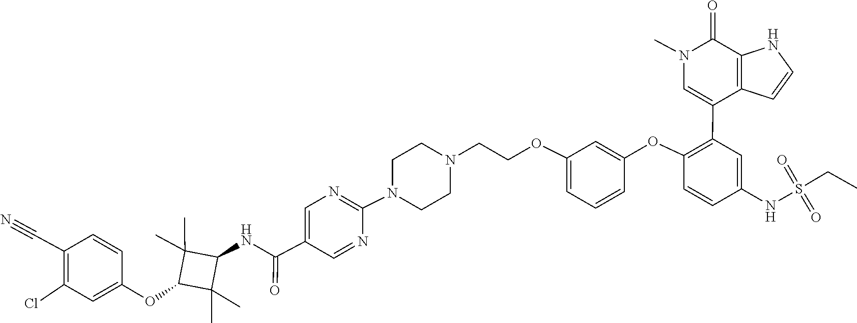 [SMILES: CCS(=O)(=O)Nc1ccc(Oc2cccc(OCCN3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1] |
| I-98 | 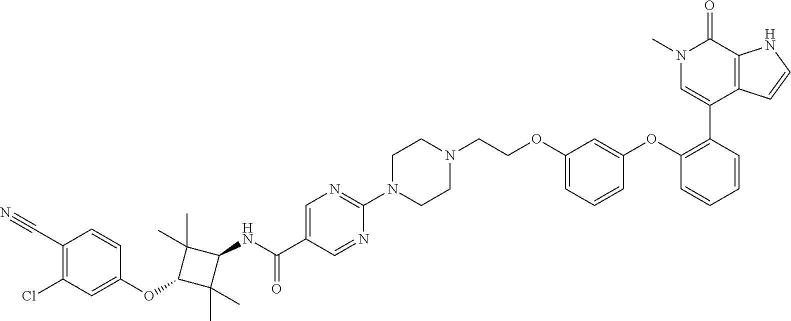 [SMILES: Cn1cc(-c2ccccc2Oc2cccc(OCCN3CCN(c4ncc(C(=O)N[C@H]5C(C)(C)[C@H](Oc6ccc(C#N)c(Cl)c6)C5(C)C)cn4)CC3)c2)c2cc[nH]c2c1=O] |
| I-99 | 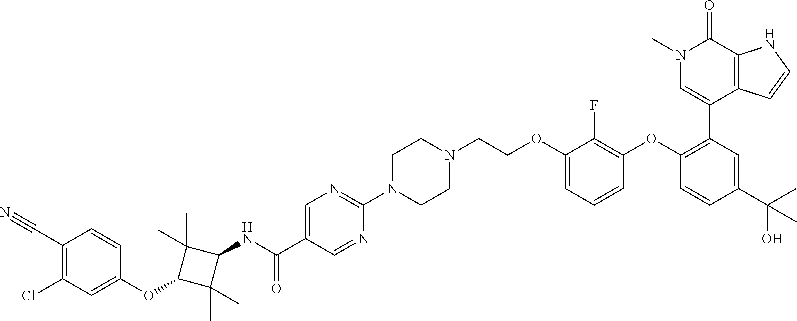 [SMILES: Cn1cc(-c2cc(C(C)(C)O)ccc2Oc2cccc(OCCN3CCN(c4ncc(C(=O)N[C@H]5C(C)(C)[C@H](Oc6ccc(C#N)c(Cl)c6)C5(C)C)cn4)CC3)c2F)c2cc[nH]c2c1=O] |
| I-100 | 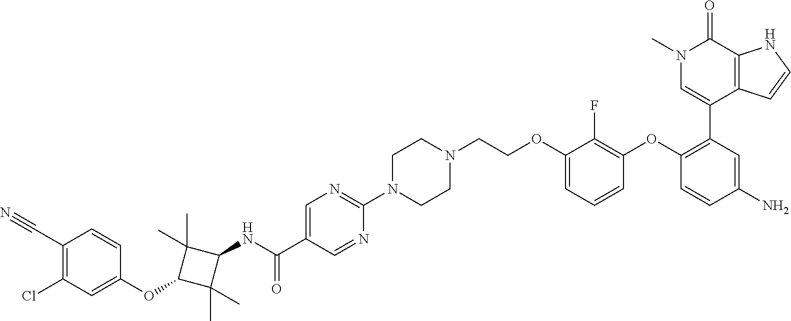 [SMILES: Cn1cc(-c2cc(N)ccc2Oc2cccc(OCCN3CCN(c4ncc(C(=O)N[C@H]5C(C)(C)[C@H](Oc6ccc(C#N)c(Cl)c6)C5(C)C)cn4)CC3)c2F)c2cc[nH]c2c1=O] |
| I-101 | 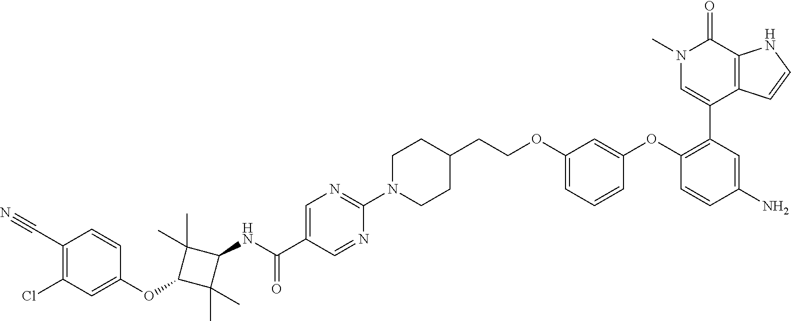 [SMILES: Cn1cc(-c2cc(N)ccc2Oc2cccc(OCCC3CCN(c4ncc(C(=O)N[C@H]5C(C)(C)[C@H](Oc6ccc(C#N)c(Cl)c6)C5(C)C)cn4)CC3)c2)c2cc[nH]c2c1=O] |
| I-102 | 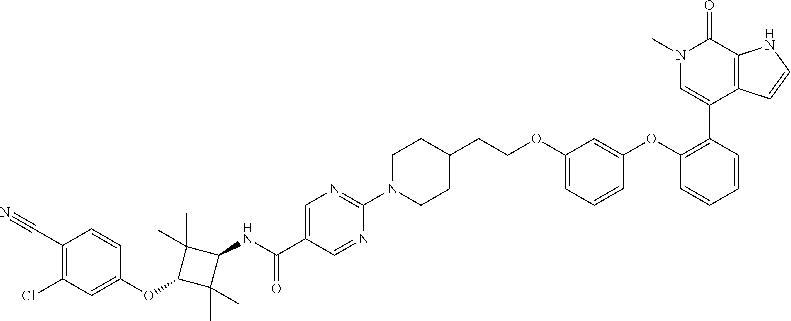 [SMILES: Cn1cc(-c2ccccc2Oc2cccc(OCCC3CCN(c4ncc(C(=O)N[C@H]5C(C)(C)[C@H](Oc6ccc(C#N)c(Cl)c6)C5(C)C)cn4)CC3)c2)c2cc[nH]c2c1=O] |
| I-103 | 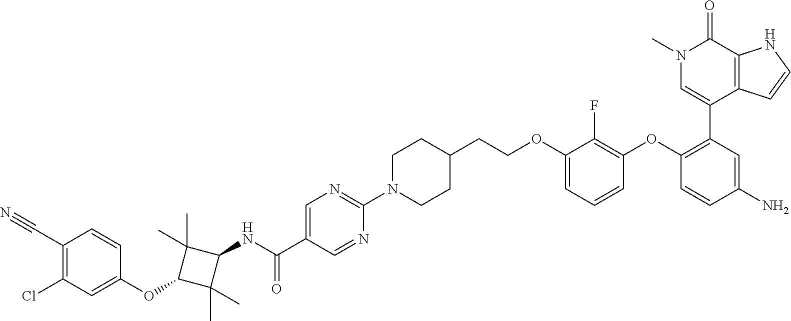 [SMILES: Cn1cc(-c2cc(N)ccc2Oc2cccc(OCCC3CCN(c4ncc(C(=O)N[C@H]5C(C)(C)[C@H](Oc6ccc(C#N)c(Cl)c6)C5(C)C)cn4)CC3)c2F)c2cc[nH]c2c1=O] |
| I-104 | 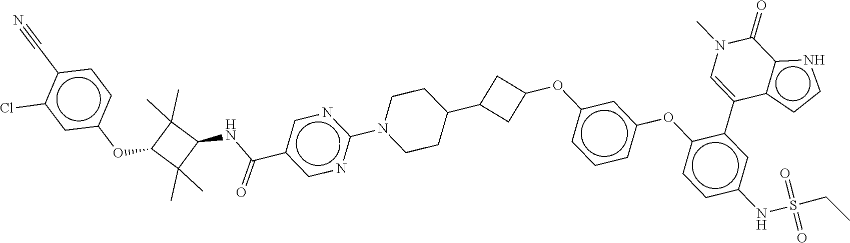 |
| I-105 | 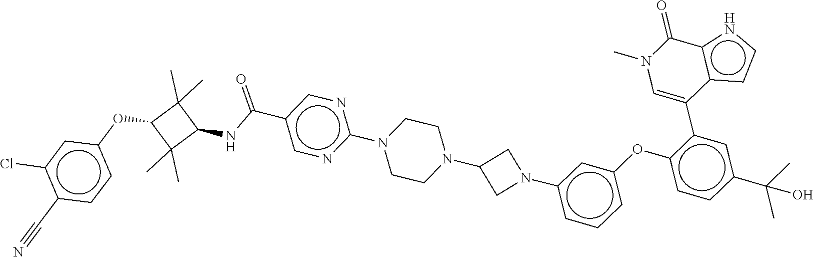 |
| I-106 | 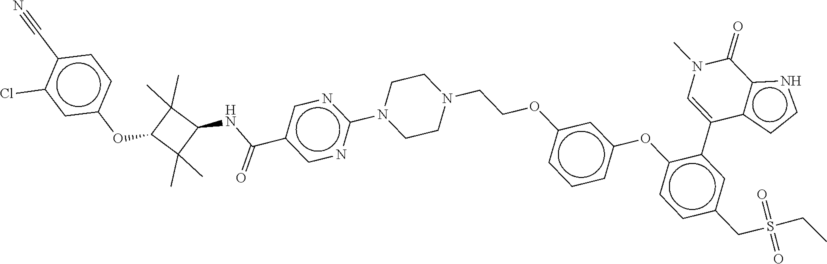 |
| I-107 | 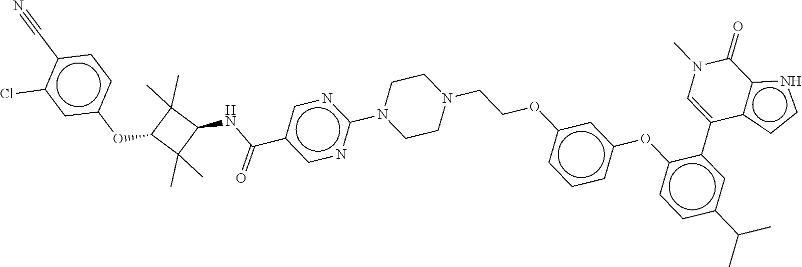 |
| I-108 | 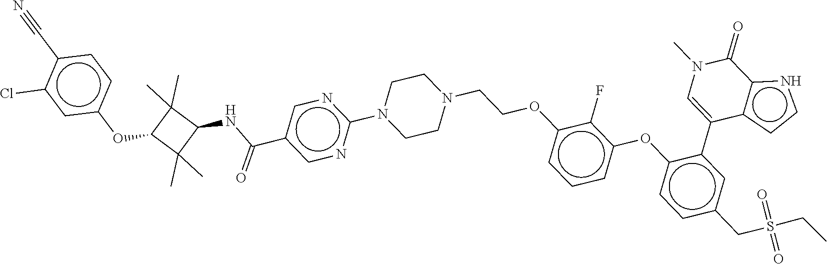 |
| I-109 | 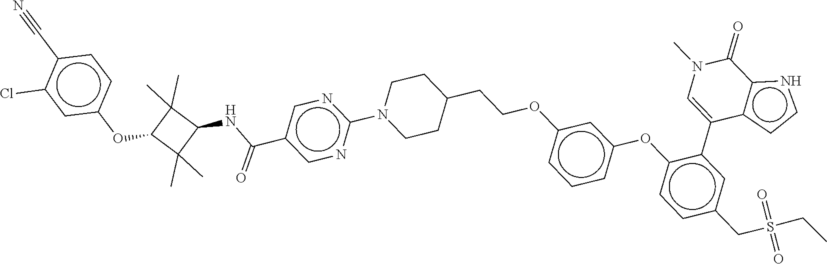 |
| I-110 | 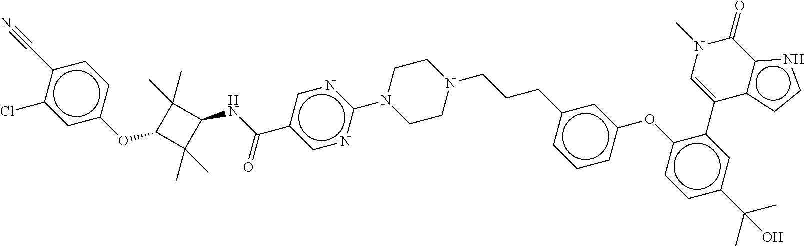 |
| I-111 | 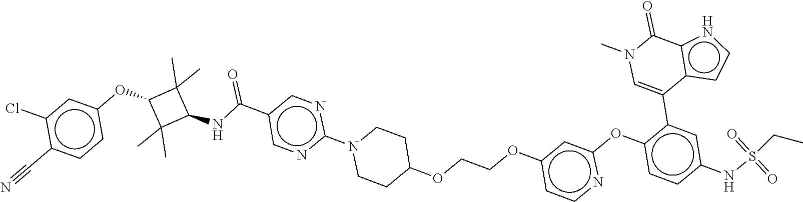 |
| I-112 | 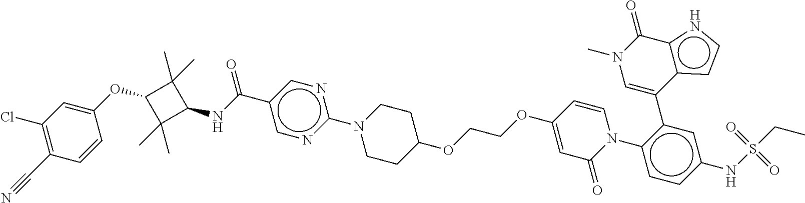 |
| I-113 | 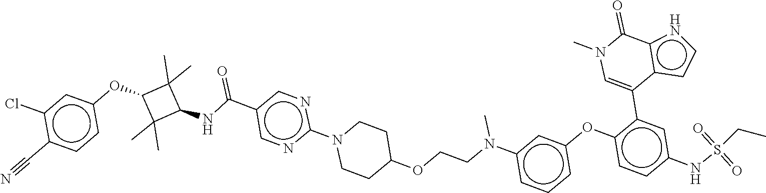 |
| I-114 | 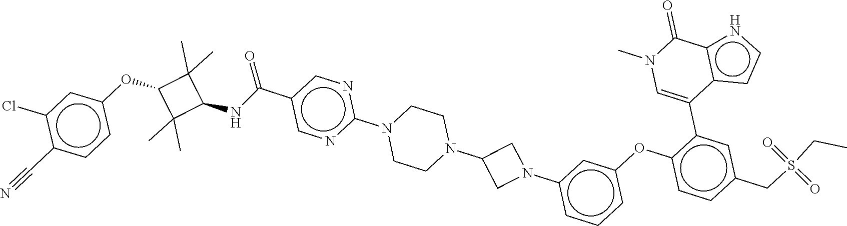 |
| I-115 | 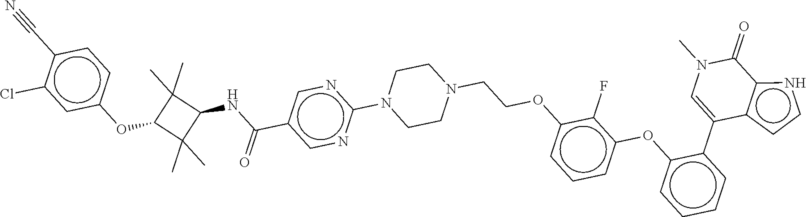 |
| I-116 | 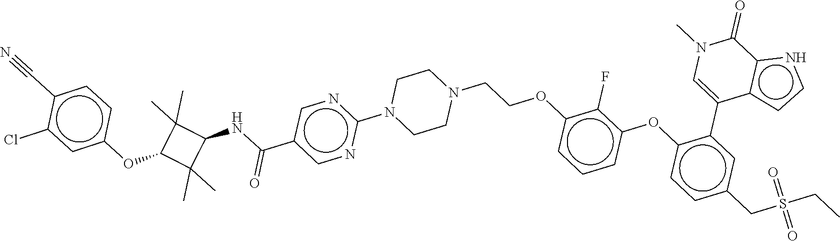 |
| I-117 | 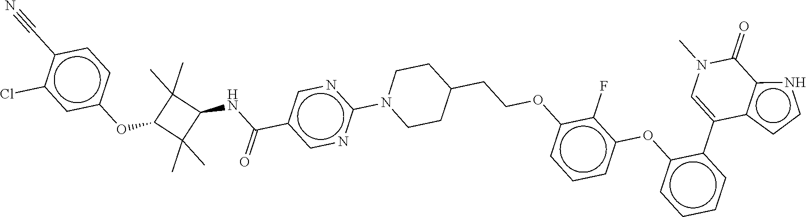 |
| I-118 | 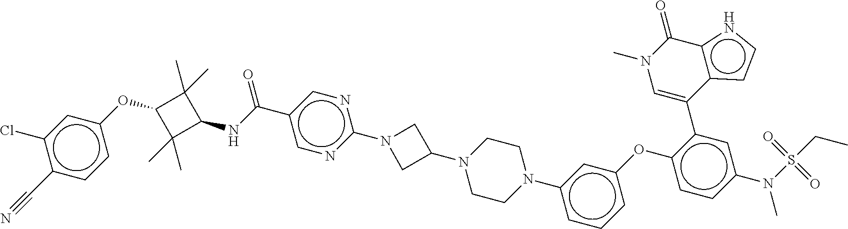 |
| I-119 | 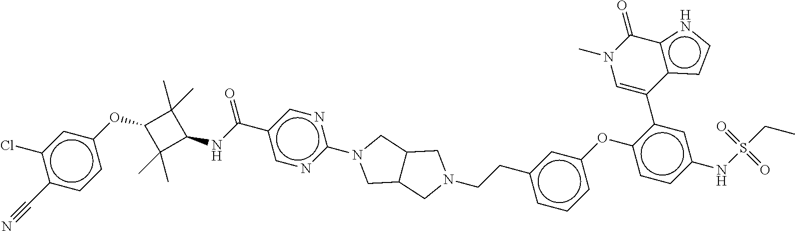 |
| I-120 | 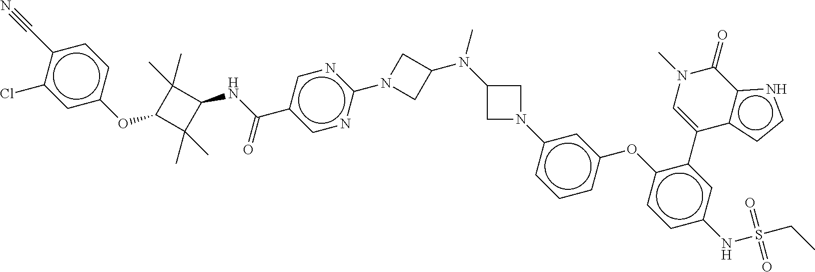 |
| I-121 | 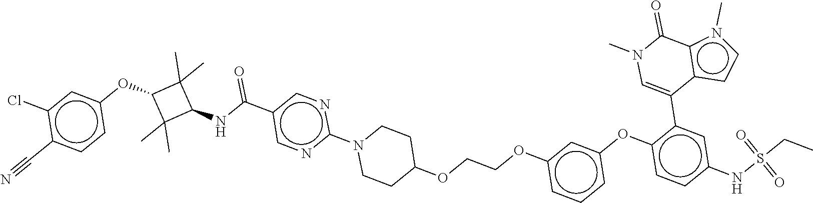 |
| I-122 | 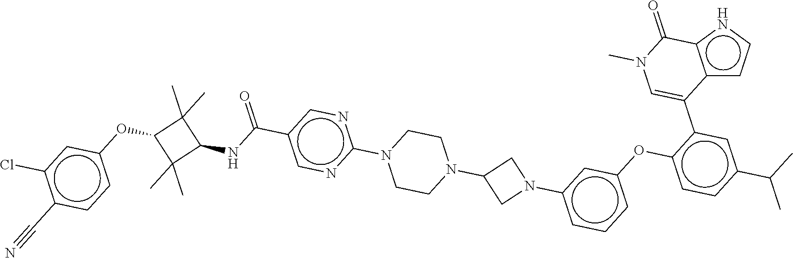 |
| I-123 | 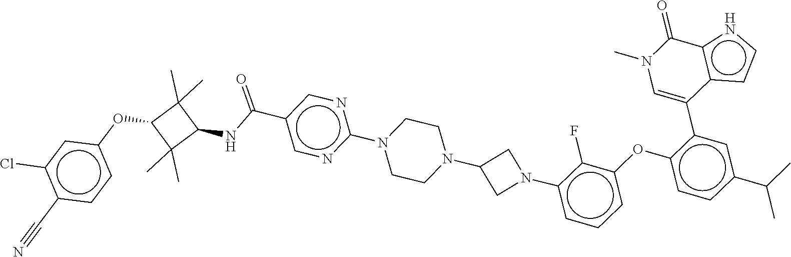 |
| I-124 | 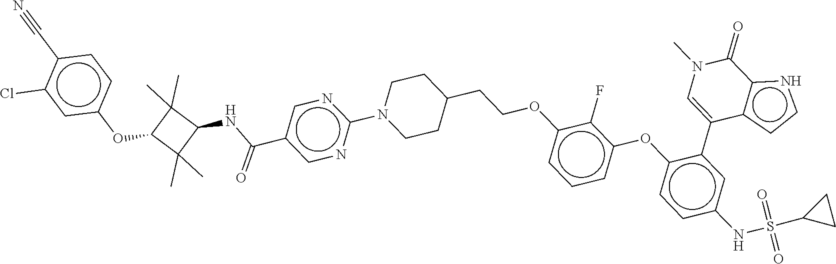 |
| I-125 | 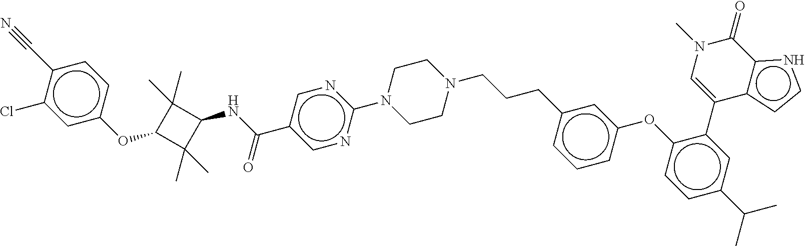 |
| I-126 | 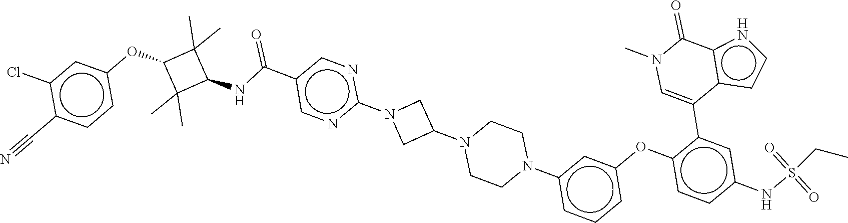 |
| I-127 | 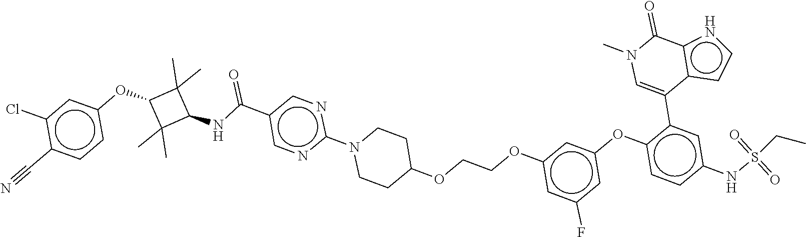 |
| I-128 | 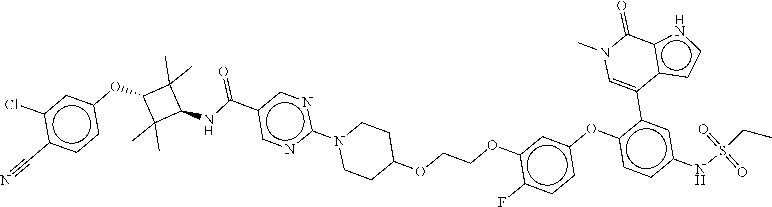 |
| I-129 | 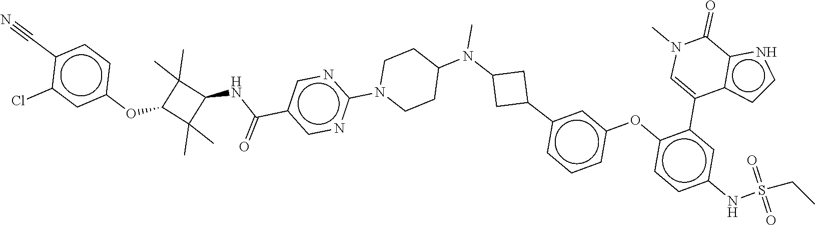 |
| I-130 | 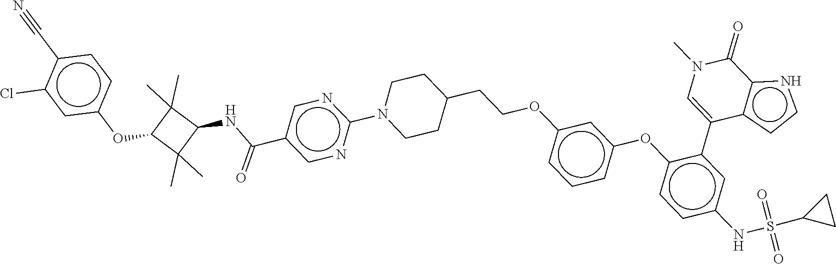 |
| I-131 | 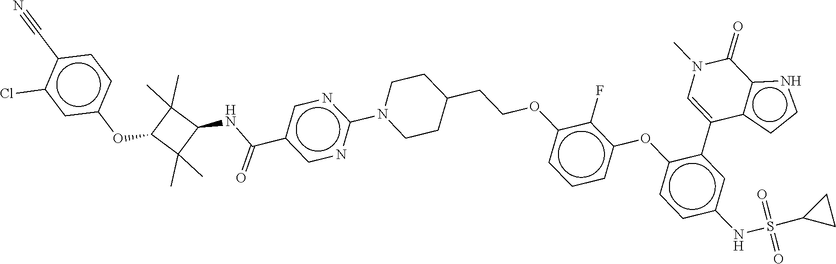 |
| I-132 | 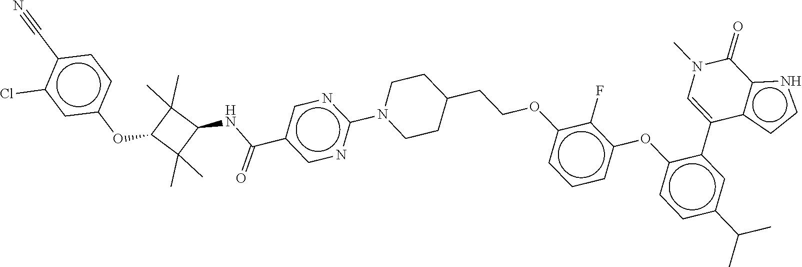 |
| I-133 | 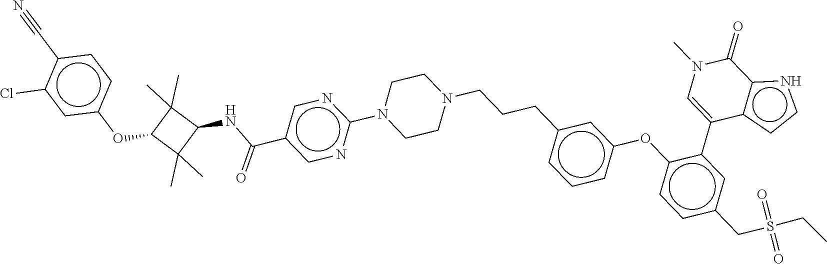 |
| I-134 | 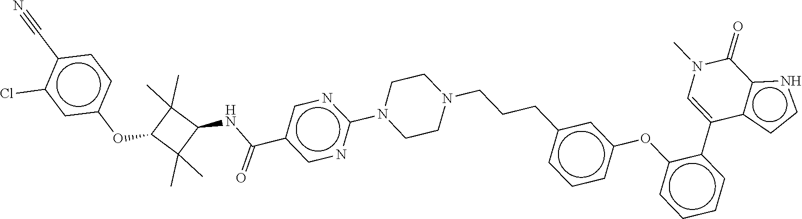 |
| I-135 | 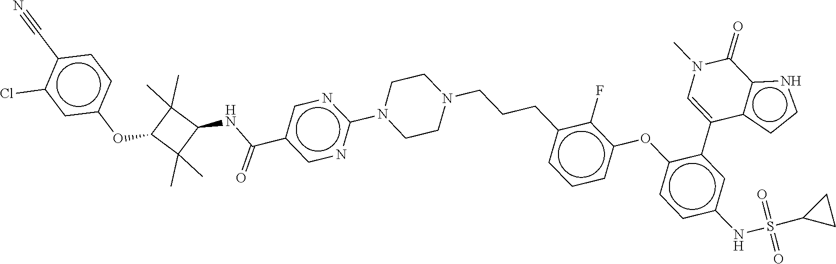 |
| I-136 | 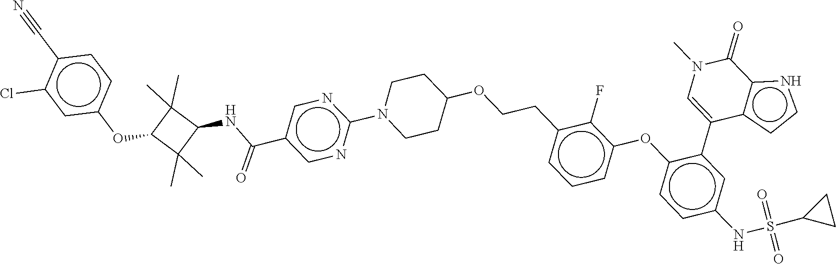 |
| I-137 | 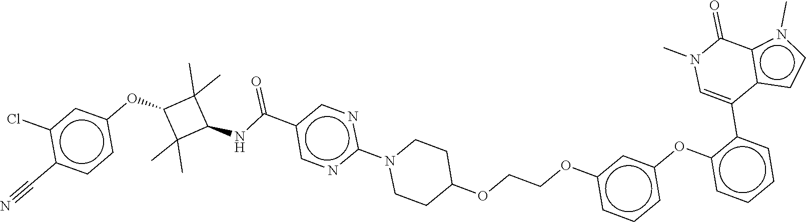 |
| I-138 | 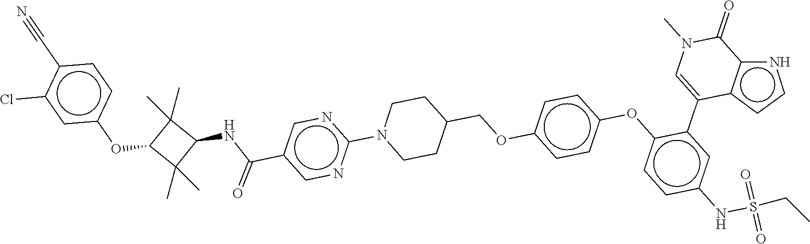 |
| I-139 | 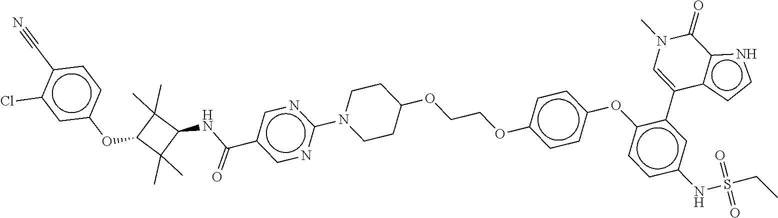 |
| I-140 | 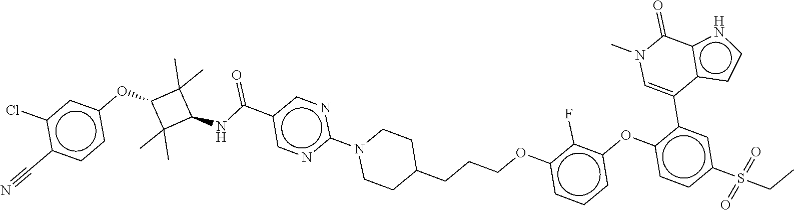 |
| I-141 | 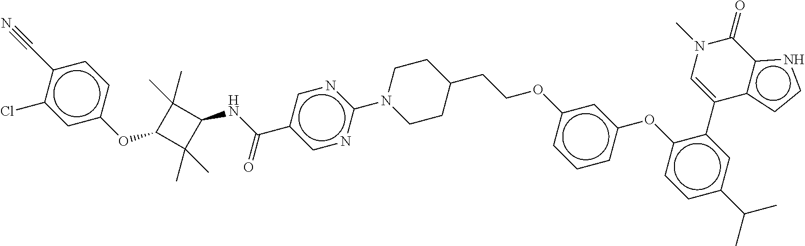 |
| I-142 | 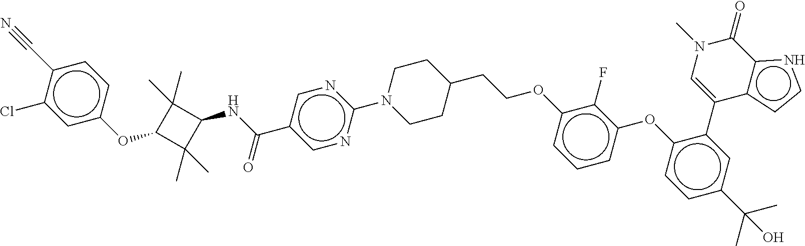 |
| I-143 | 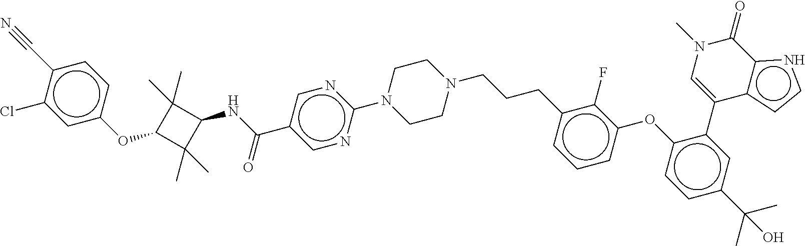 |
| I-144 | 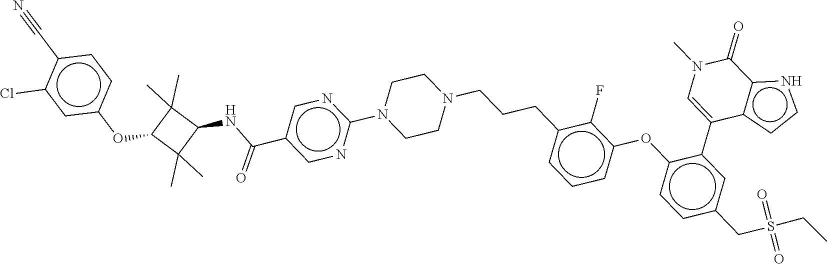 |
| I-145 | 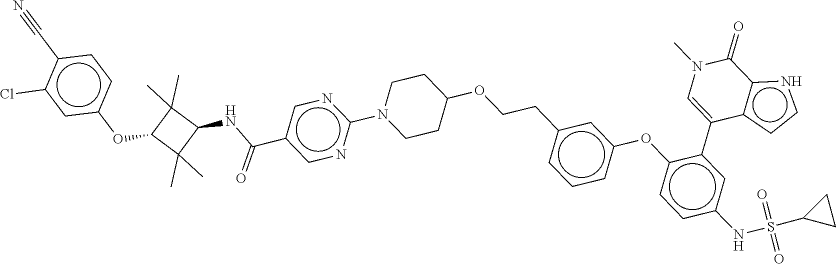 |
| I-146 | 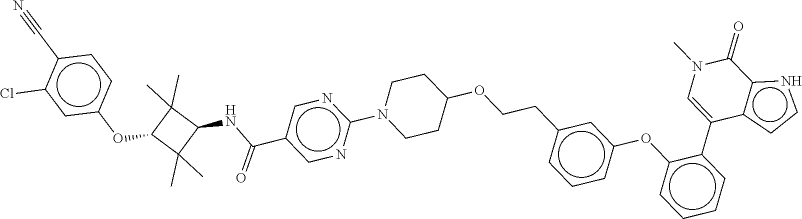 |
| I-147 | 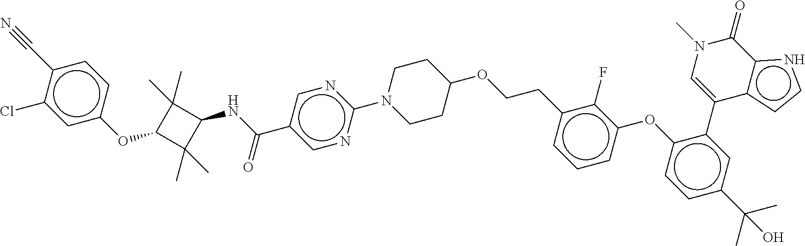 |
| I-148 | 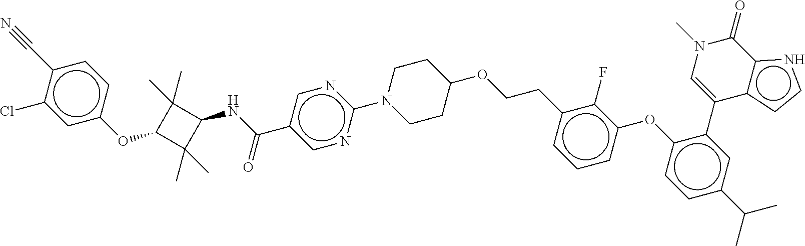 |
| I-149 | 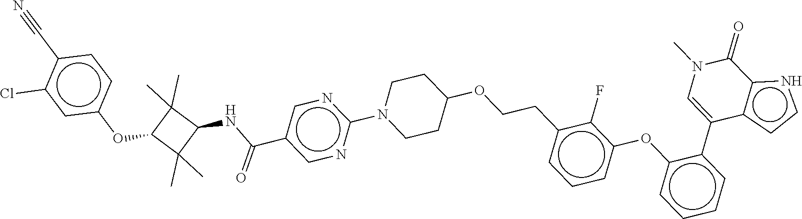 |
| I-150 | 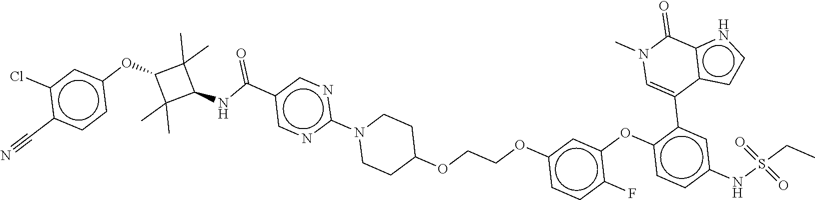 |
| I-151 | 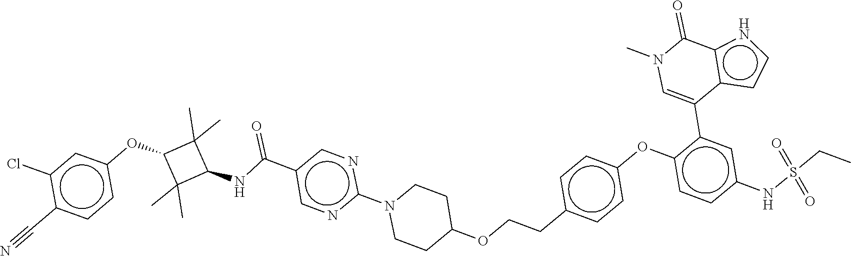 |
| I-152 | 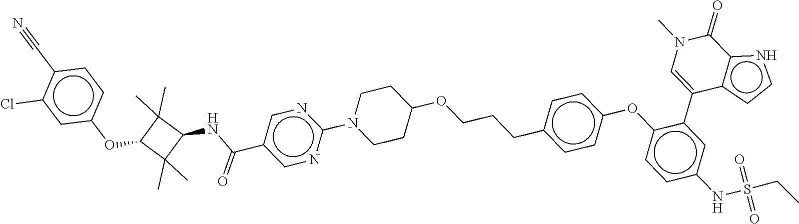 |
| I-153 | 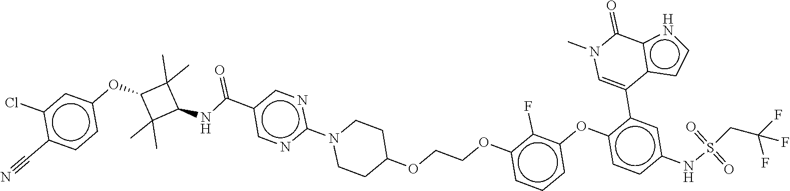 |
| I-154 | 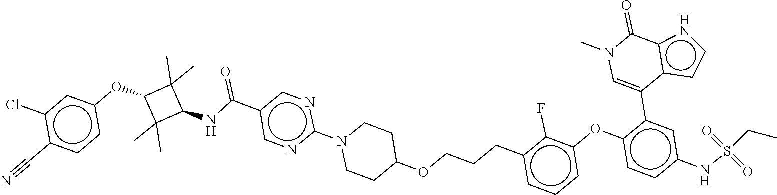 |
| I-155 | 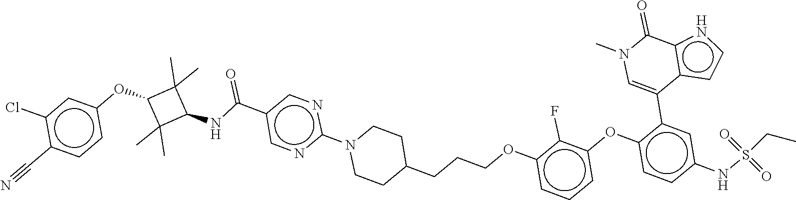 |
| I-156 | 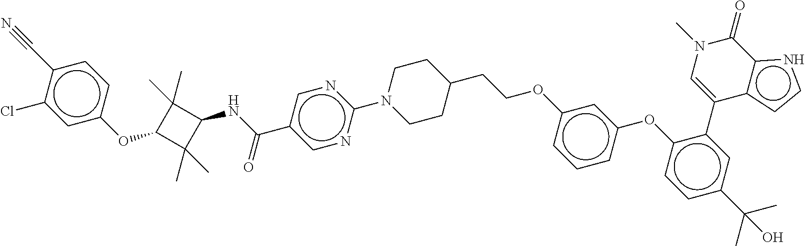 |
| I-157 | 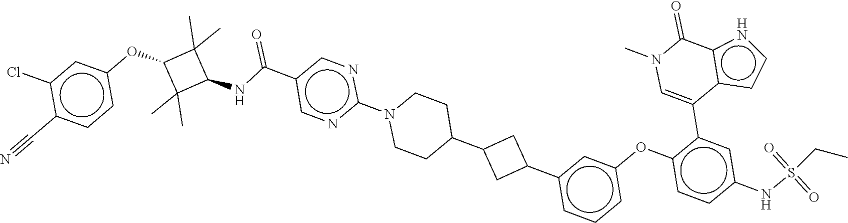 |
| I-158 | 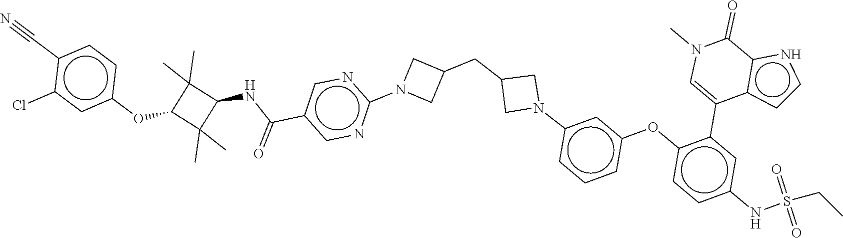 |
| I-159 | 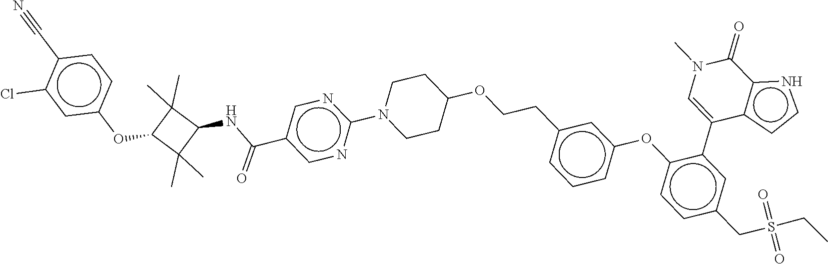 |
| I-160 | 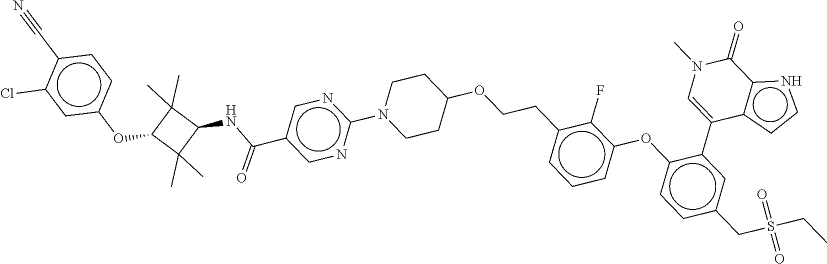 |
| I-161 | 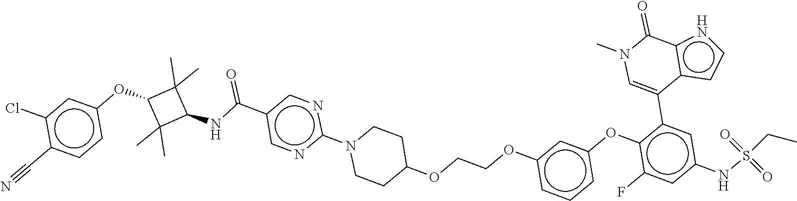 |
| I-162 | 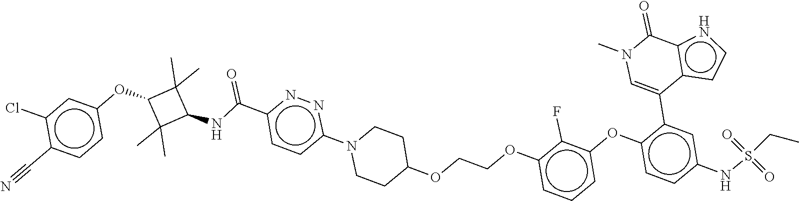 |
| I-163 | 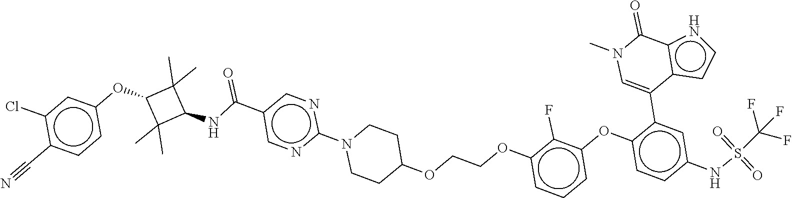 |
| I-164 | 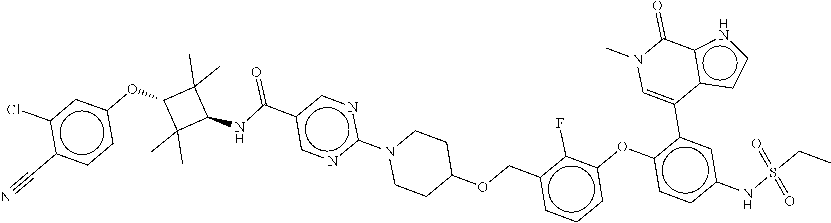 |
| I-165 | 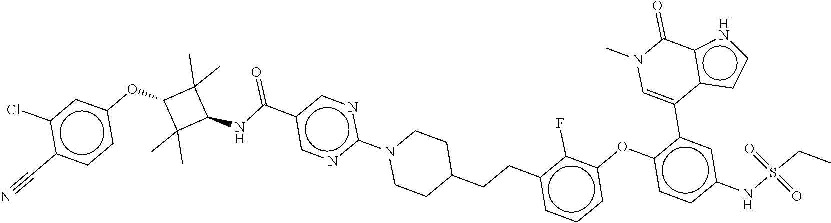 |
| I-166 | 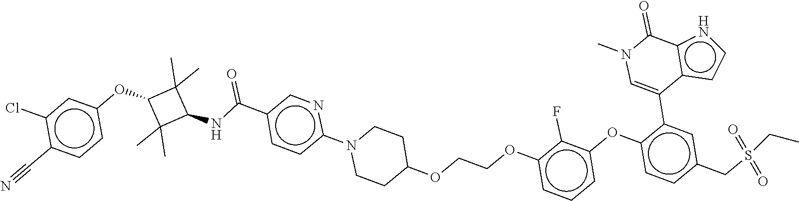 |
| I-167 | 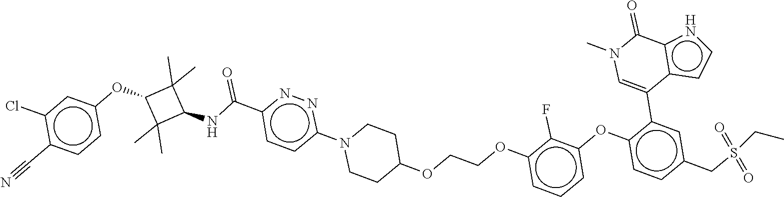 |
| I-168 | 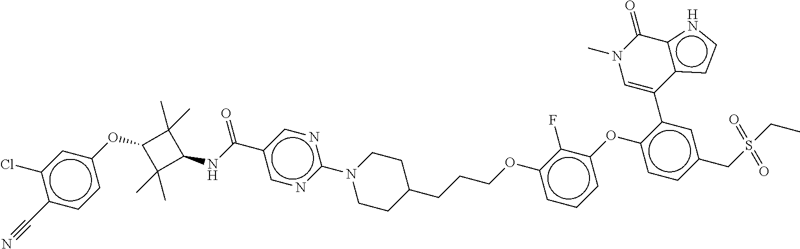 |
| I-169 | 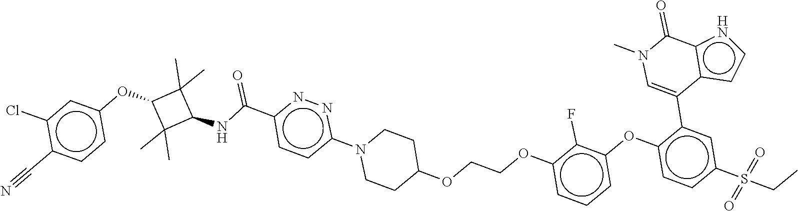 |
| I-170 | 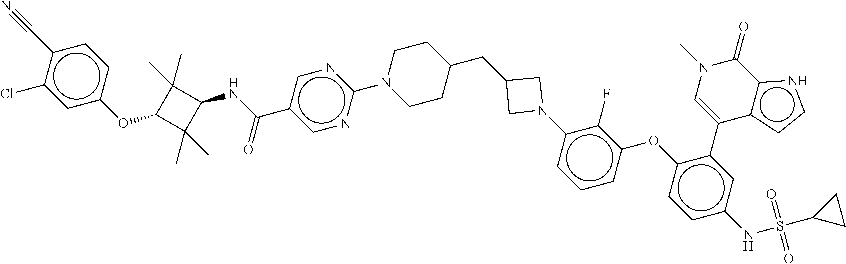 |
| I-171 | 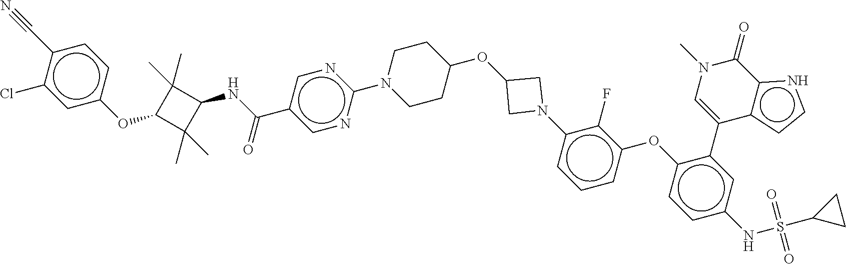 |
| I-172 | 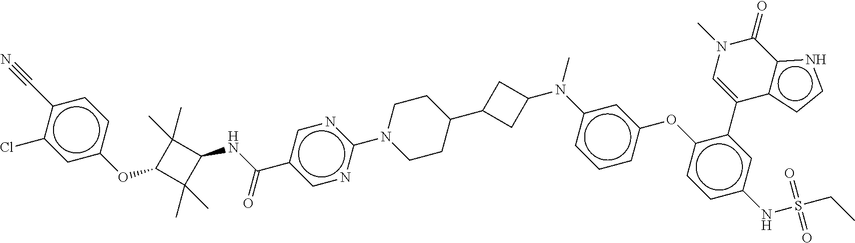 |
| I-173 | 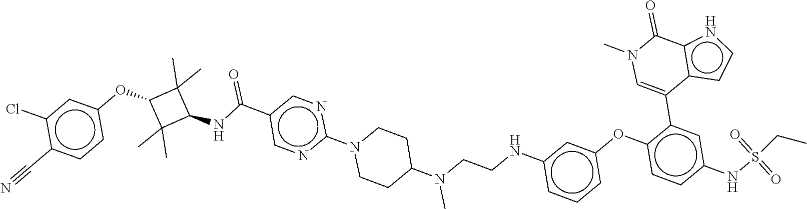 |
| I-174 | 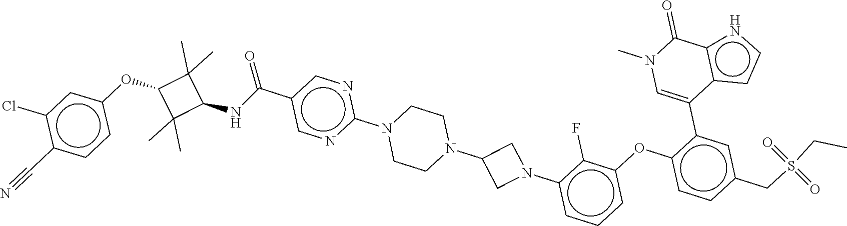 |
| I-175 | 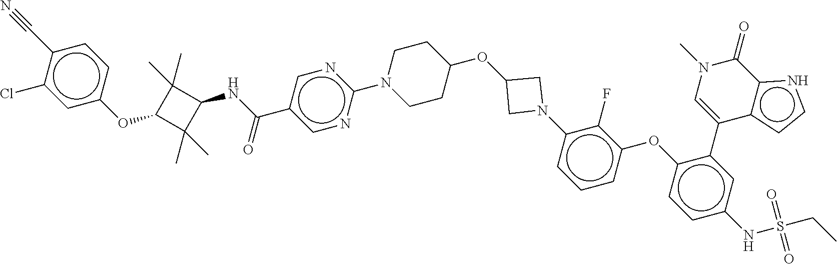 |
| I-176 | 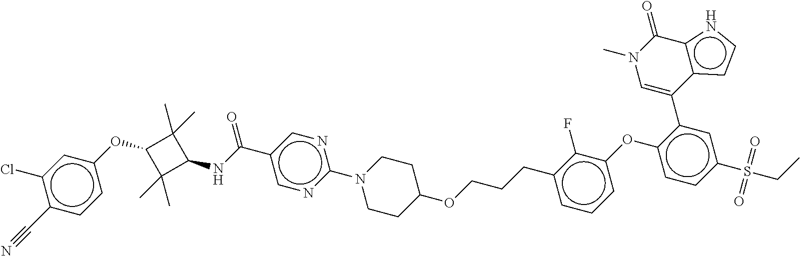 |
| I-177 | 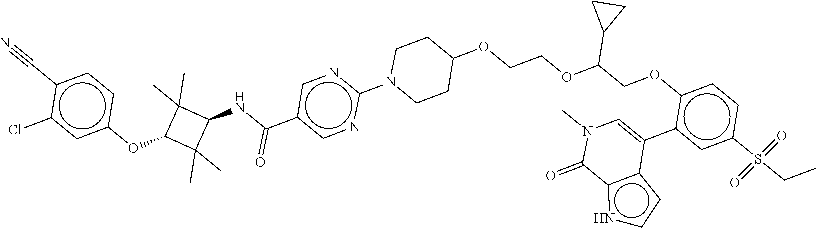 |
| I-178 | 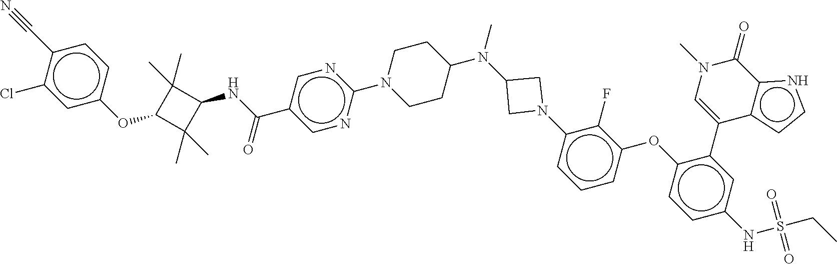 |
| I-179 | 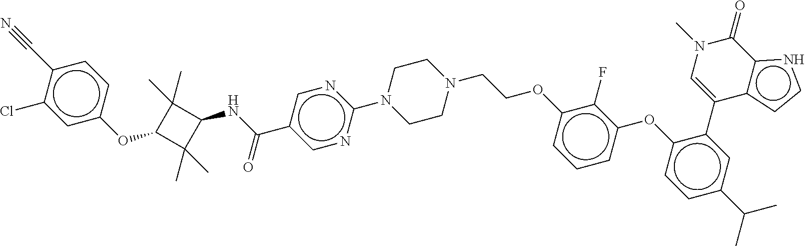 |
| I-180 | 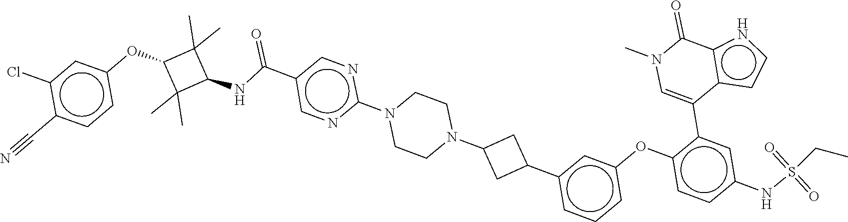 |
| I-181 | 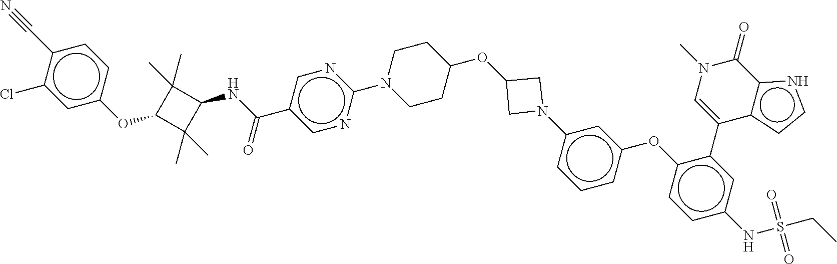 |
| I-182 | 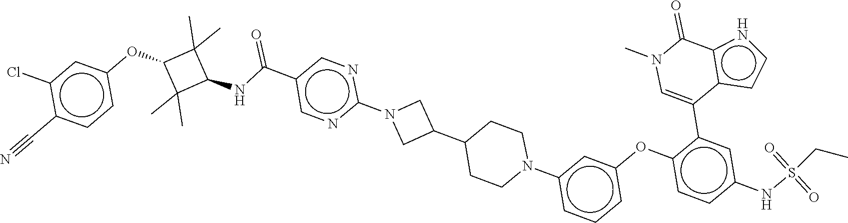 |
| I-183 | 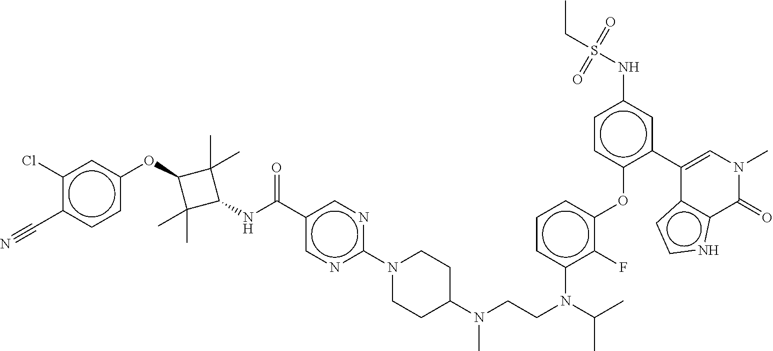 |
| I-184 | 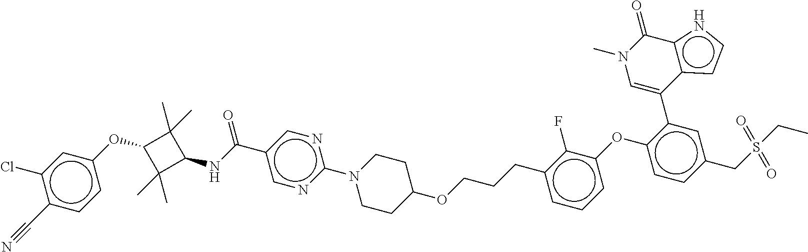 |
| I-185 | 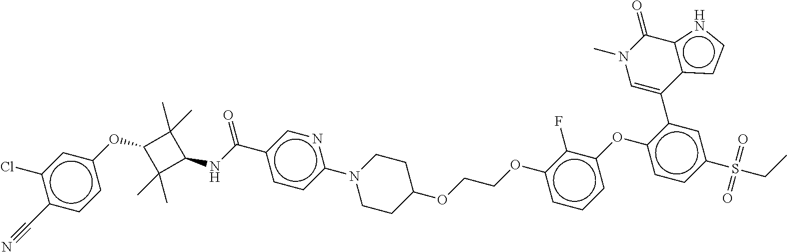 |
| I-186 | 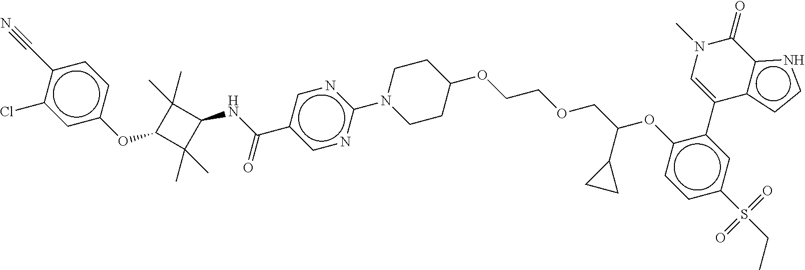 |
| I-187 | 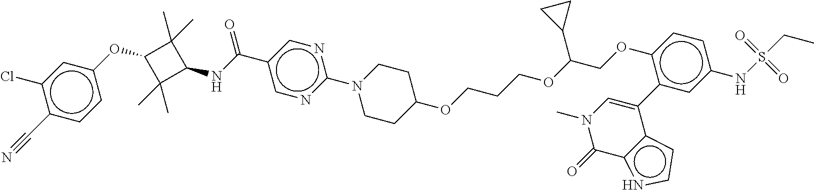 |
| I-188 | 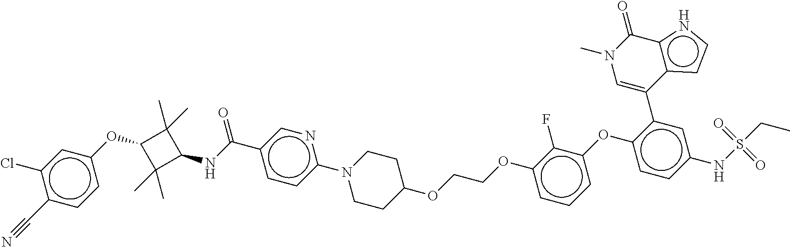 |
| I-189 | 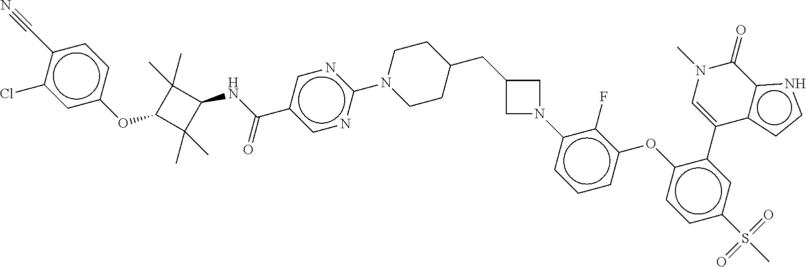 |
| I-190 | 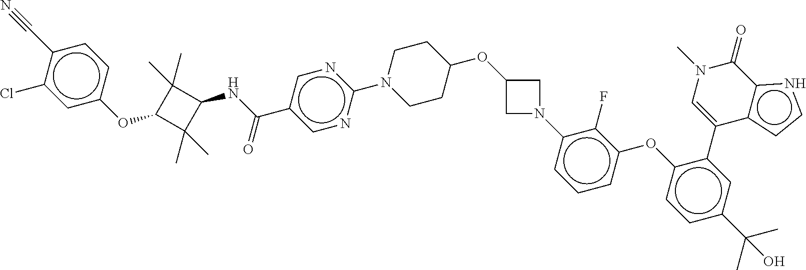 |
| I-191 | 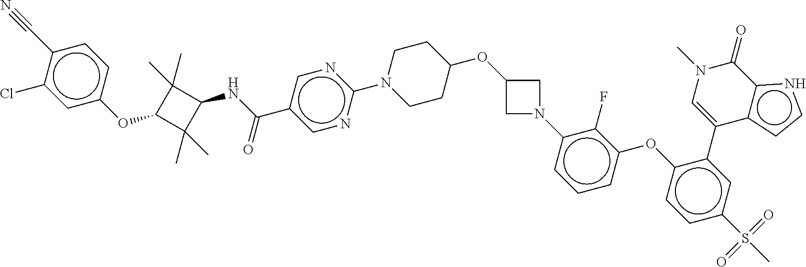 |
| I-192 | 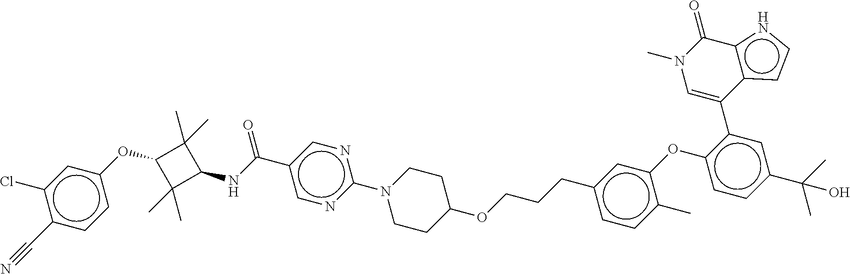 |
| I-193 | 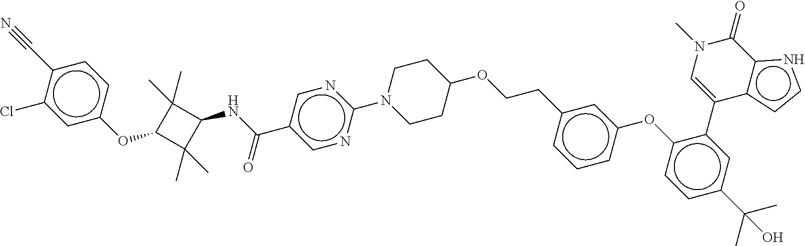 |
| I-194 | 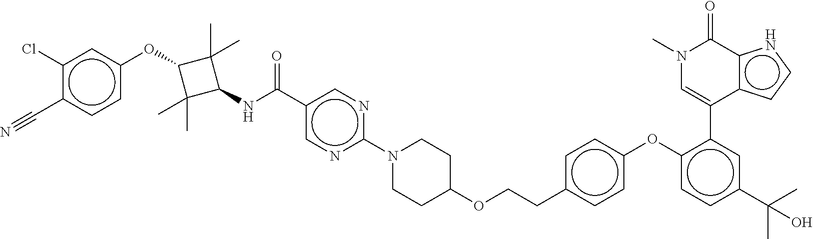 |
| I-195 | 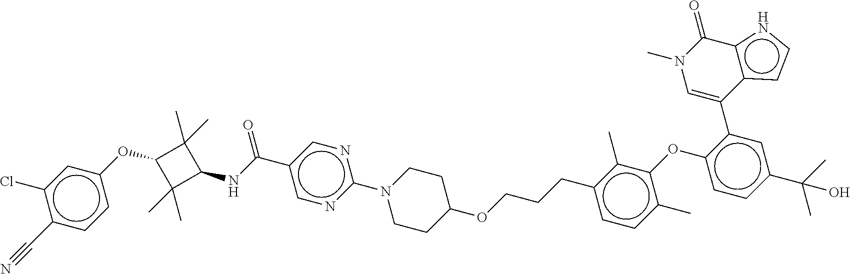 |
| I-196 | 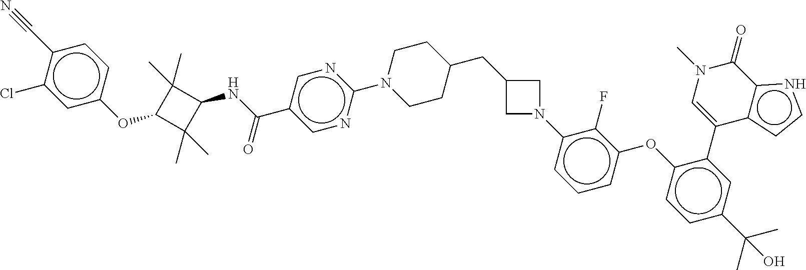 |
| I-197 | 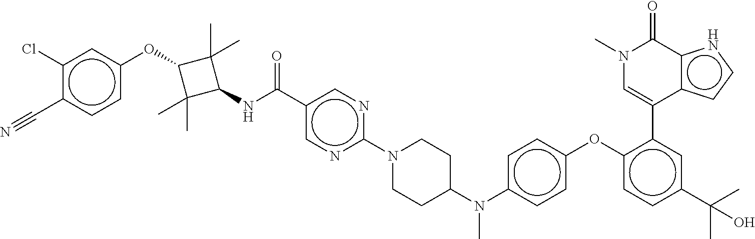 |
| I-198 | 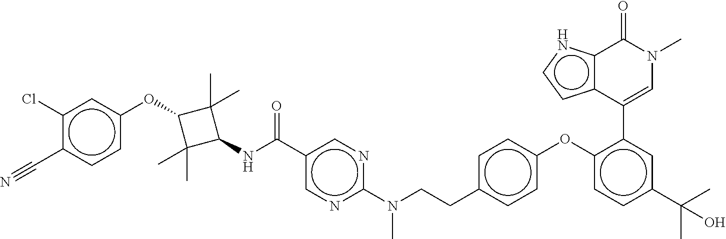 |
| I-199 | 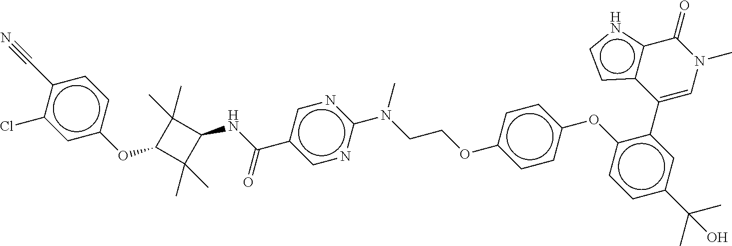 |
| I-200 | 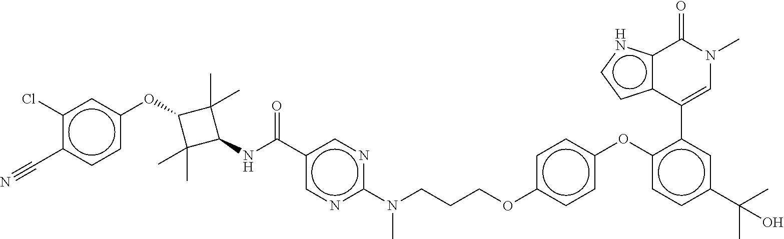 |
| I-201 | 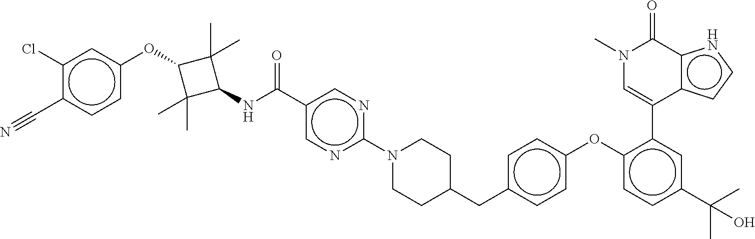 |
| I-202 | 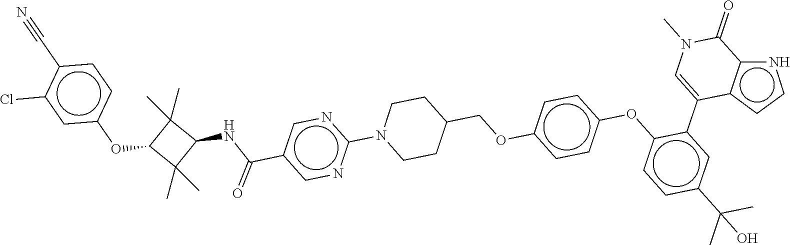 |
| I-203 | 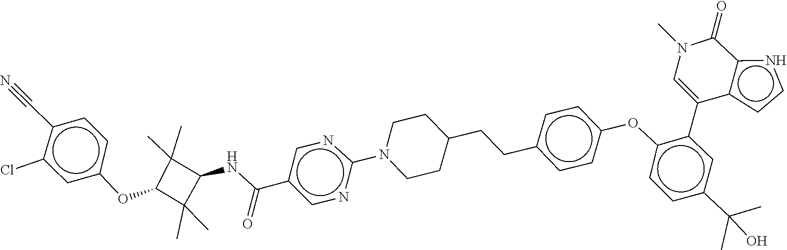 |
| I-204 | 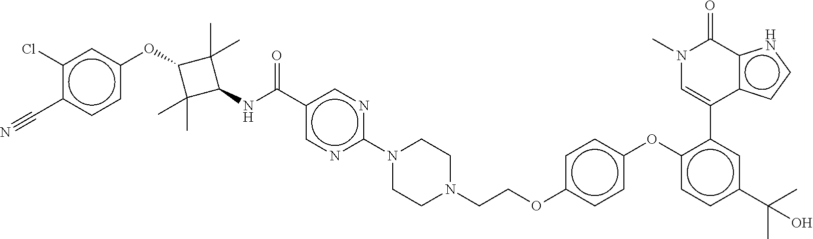 |
| I-205 | 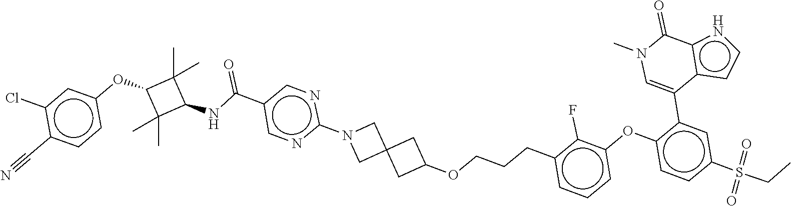 |
| I-206 | 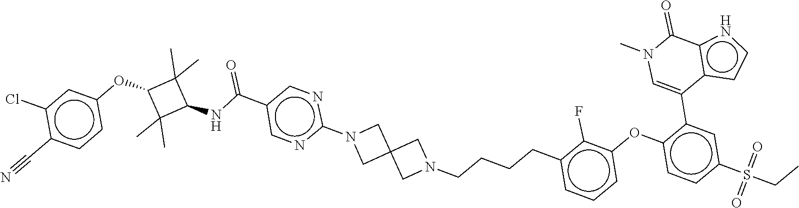 |
| I-207 | 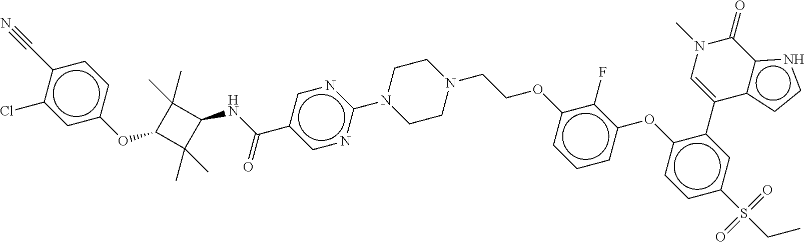 |
| I-208 | 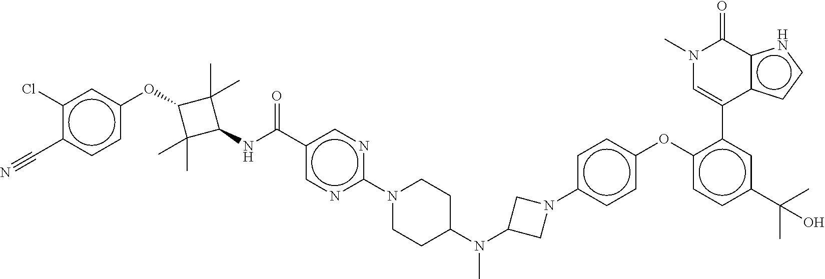 |
| I-209 | 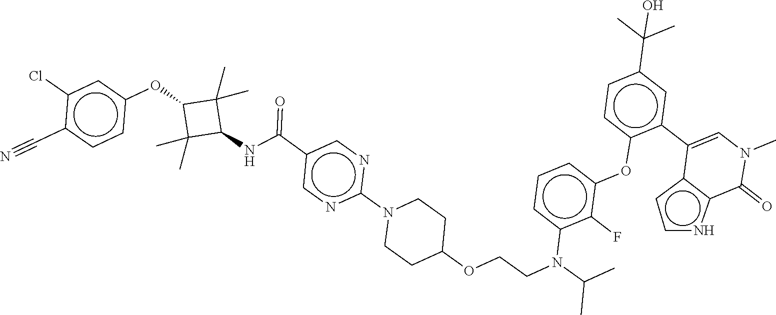 |
| I-210 | 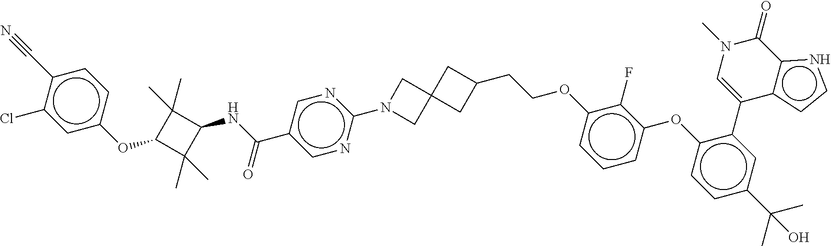 |
| I-211 | 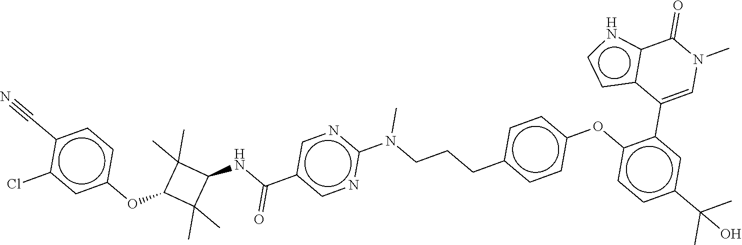 |
| I-212 | 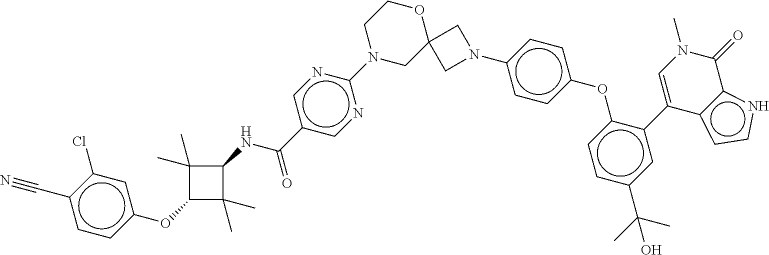 |
| I-213 | 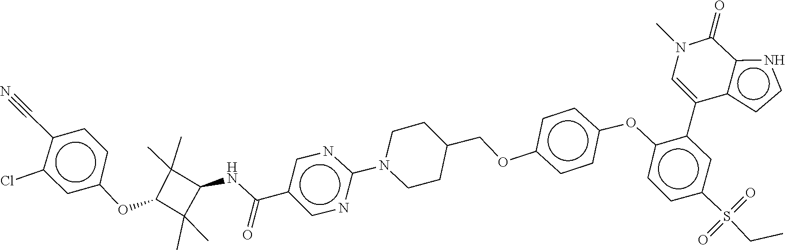 |
| I-214 | 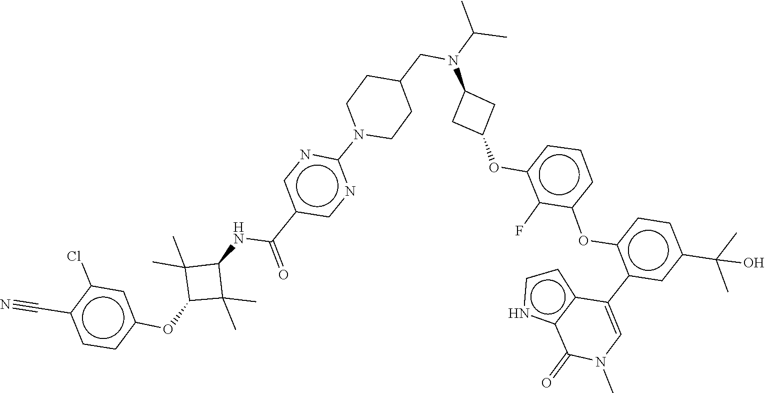 |
| I-215 | 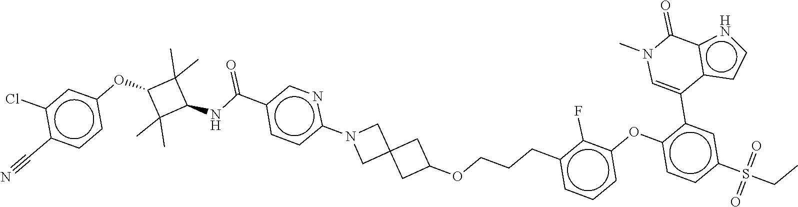 |
| I-216 | 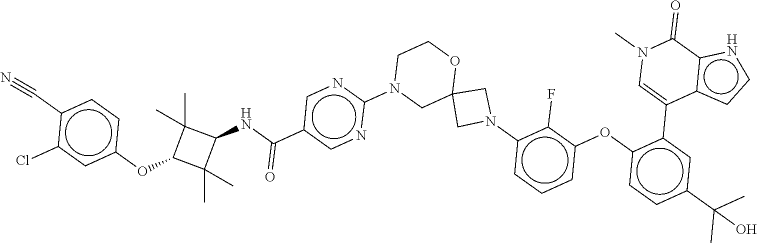 |
| I-217 | 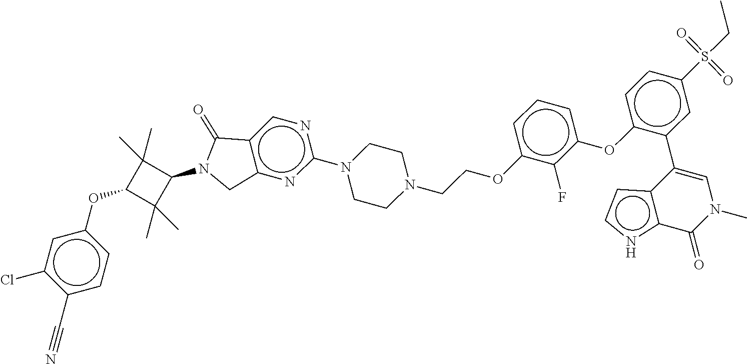 |
| I-218 | 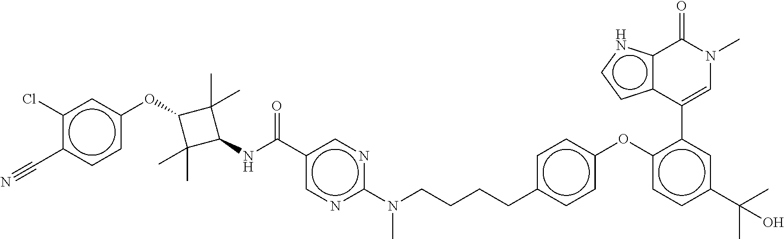 |
| I-219 | 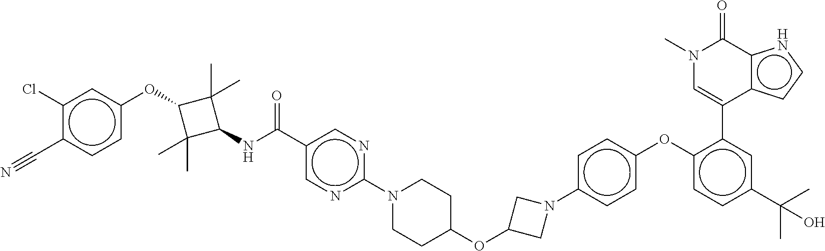 |
| I-220 | 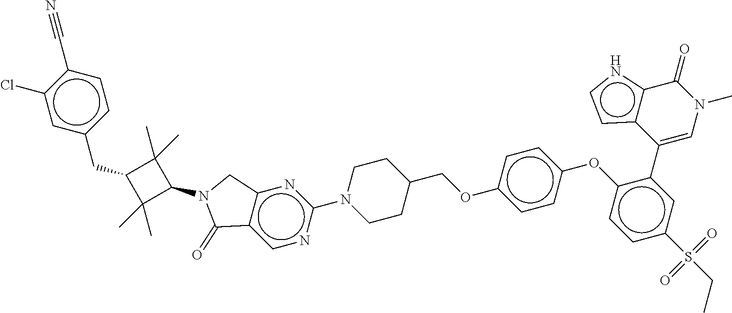 |
| I-221 | 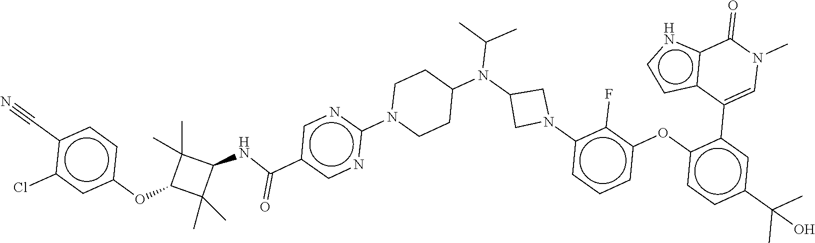 |
| I-222 | 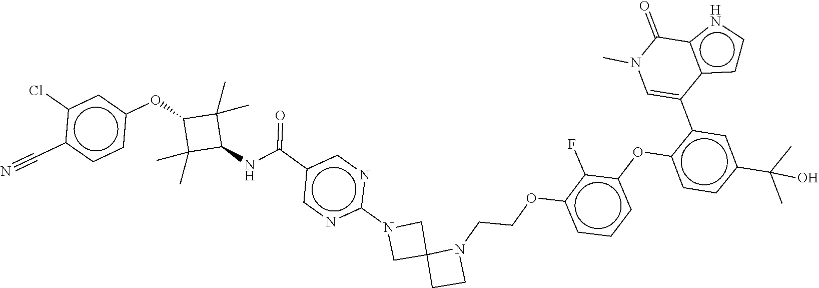 |
| I-223 | 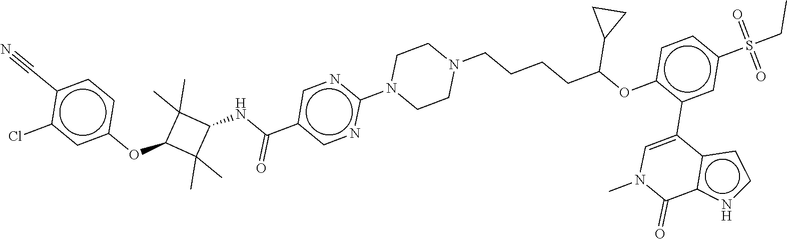 |
| I-224 | 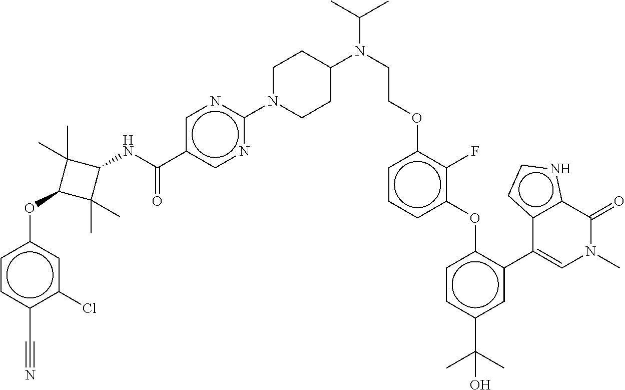 |
| I-225 | 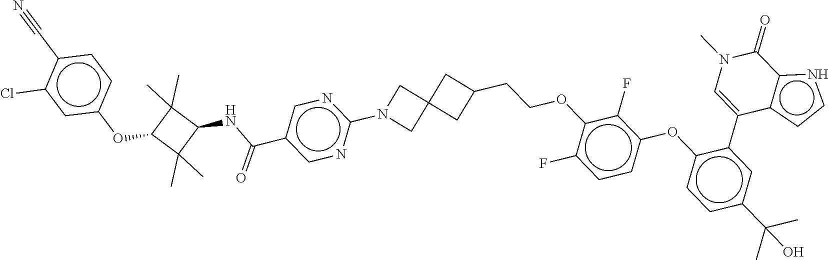 |
| I-226 | 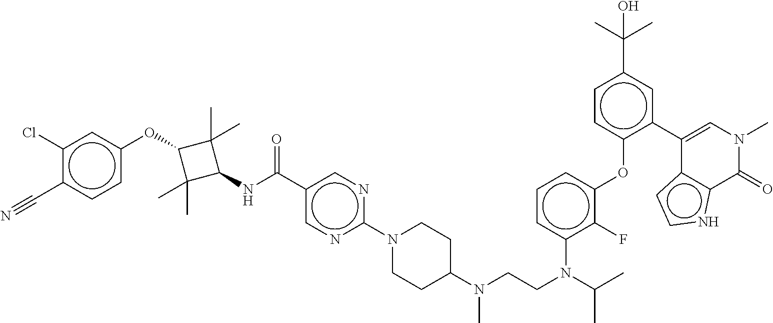 |
| I-227 | 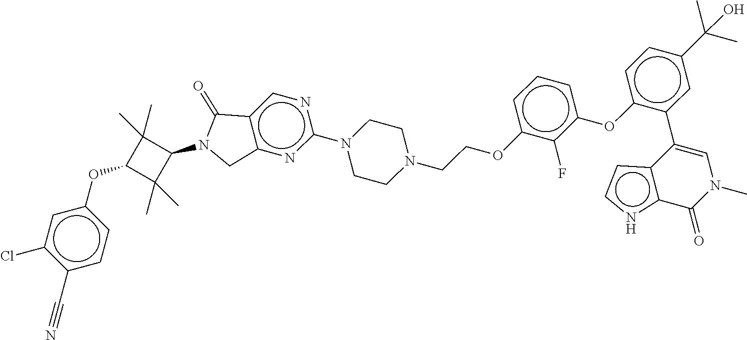 |
| I-228 | 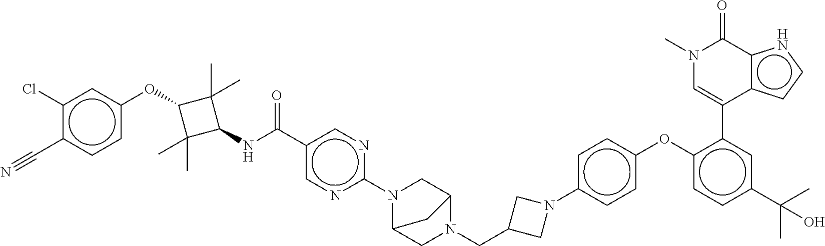 |
| I-229 | 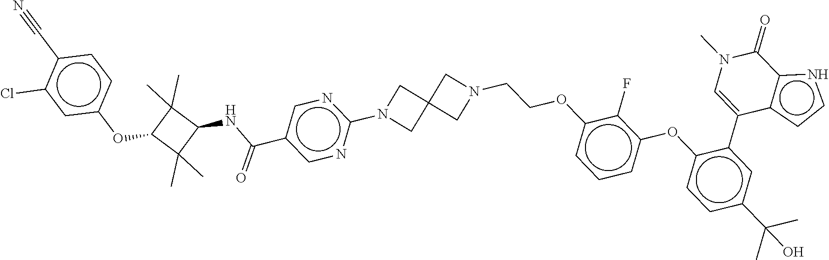 |
| I-230 | 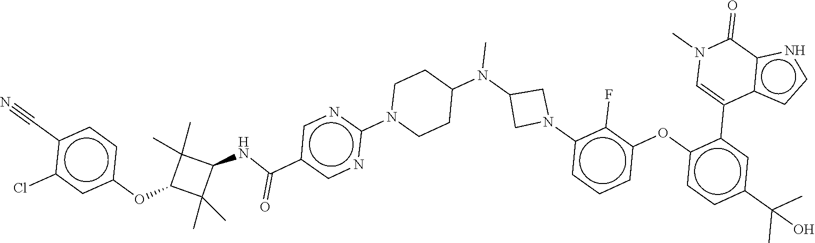 |
| I-231 | 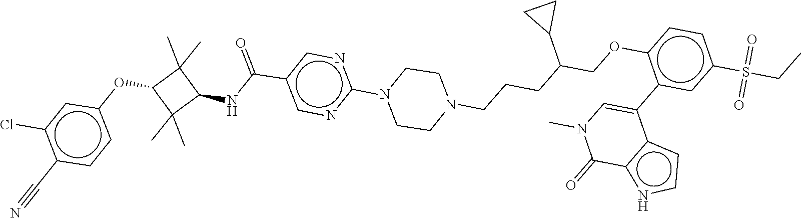 |
| I-232 | 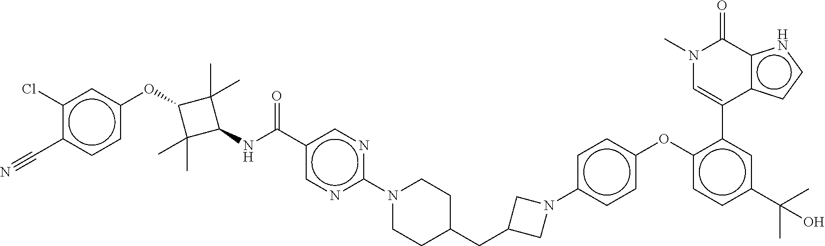 |
| I-233 | 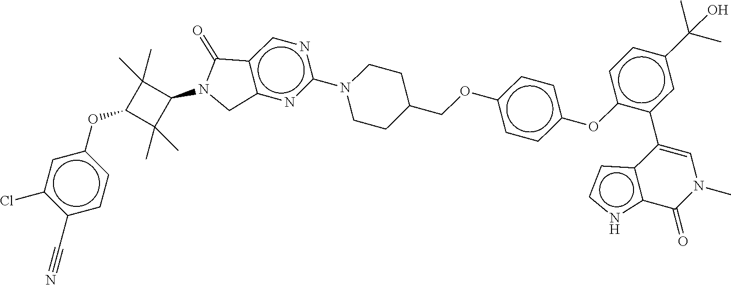 |
| I-234 | 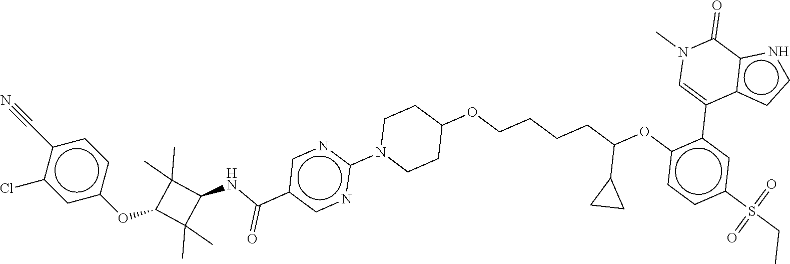 |
| I-235 | 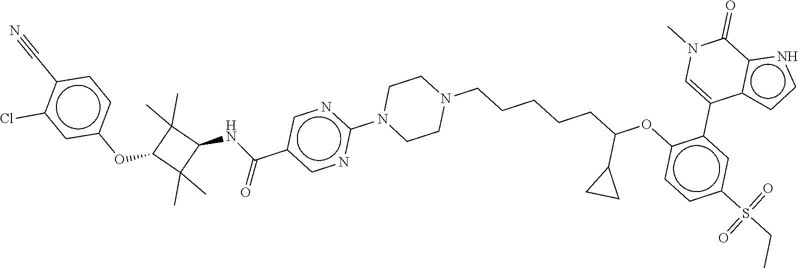 |
| I-236 | 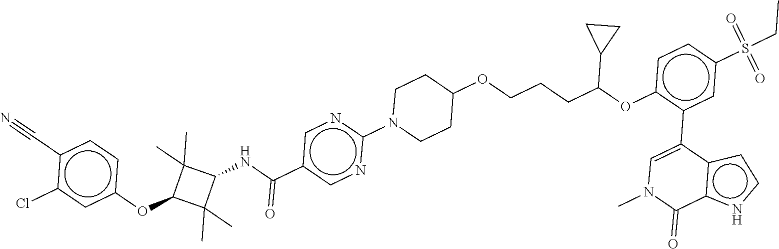 |
| I-237 | 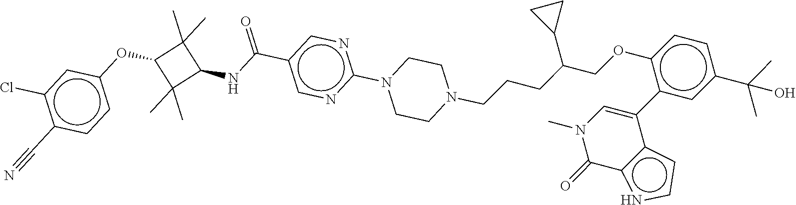 |
| I-238 | 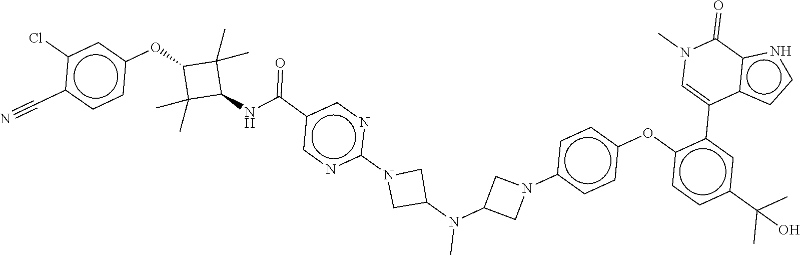 |
| I-239 | 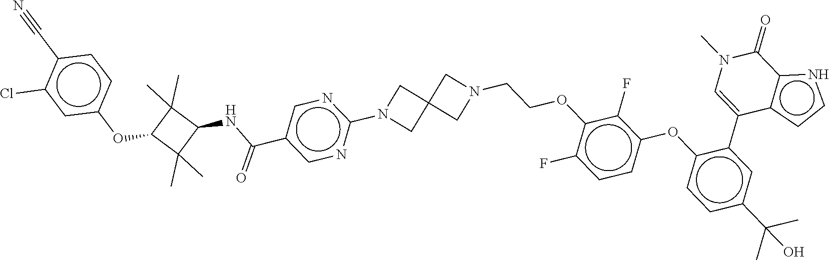 |
| I-240 | 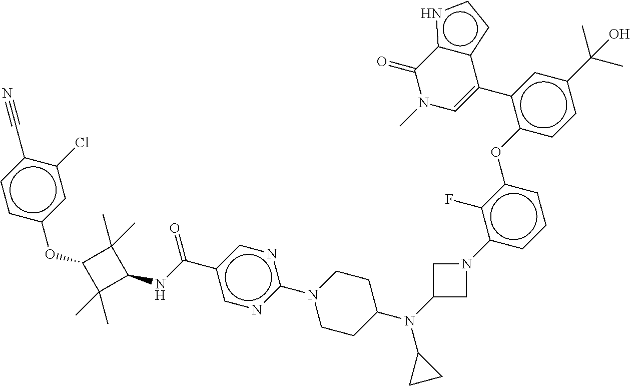 |
| I-241 | 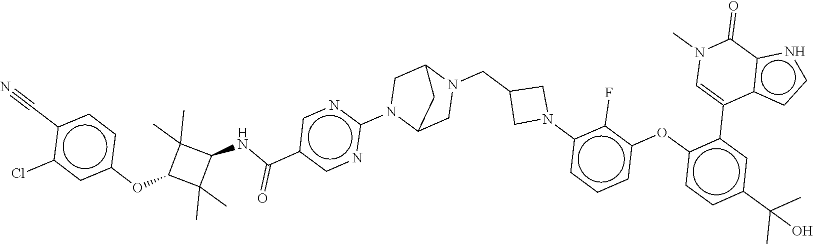 |
| I-242 | 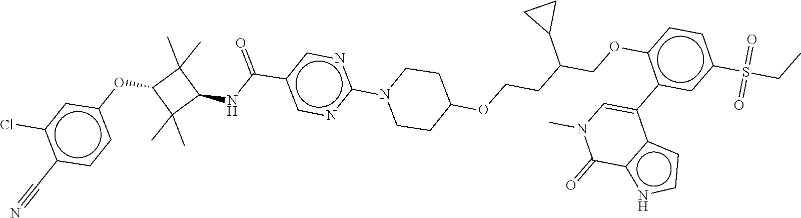 |
| I-243 | 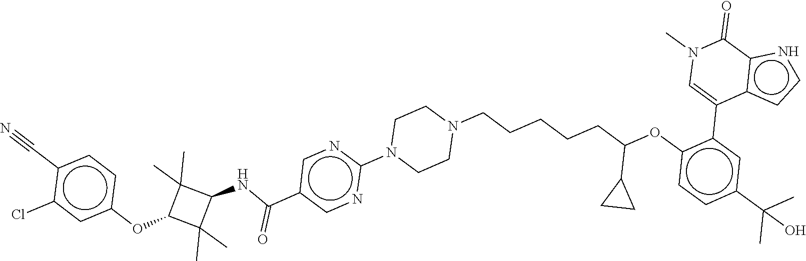 |
| I-244 | 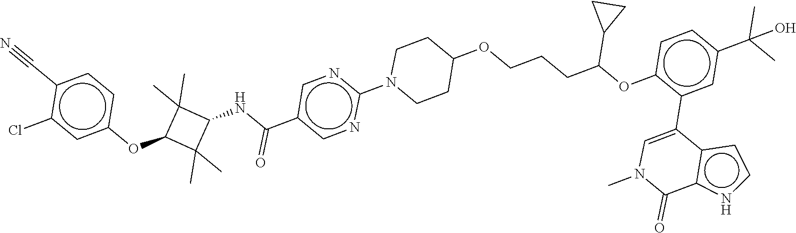 |
| I-245 | 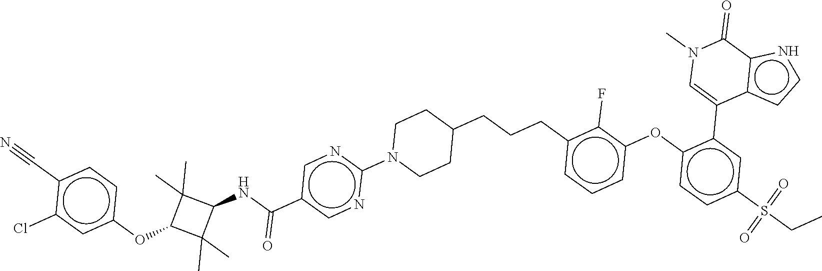 |
| I-246 | 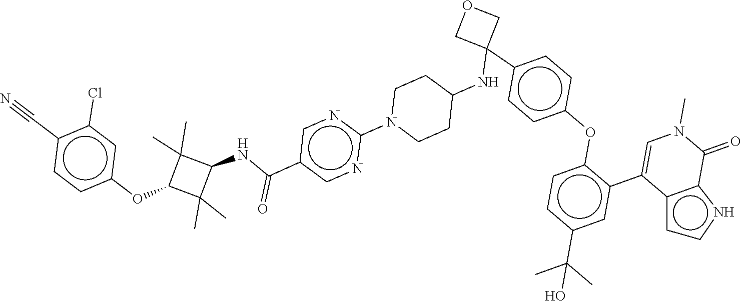 |
| I-247 | 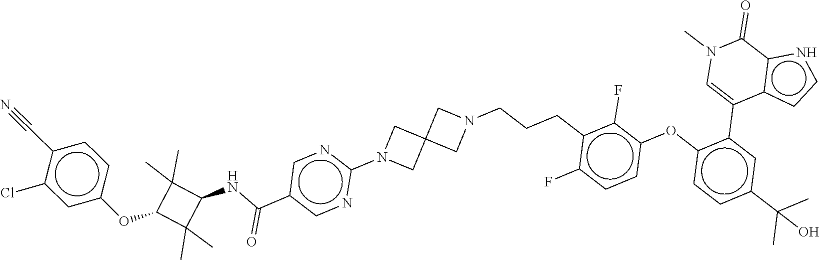 |
| I-248 | 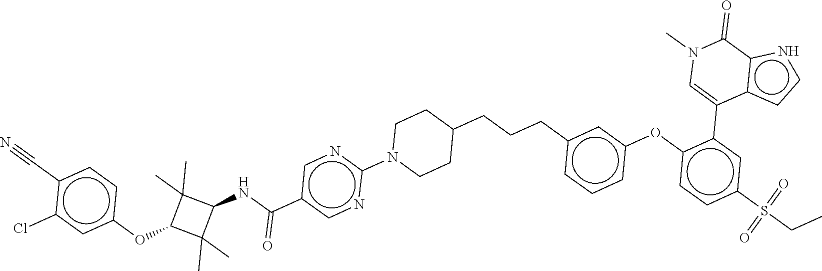 |
| I-249 | 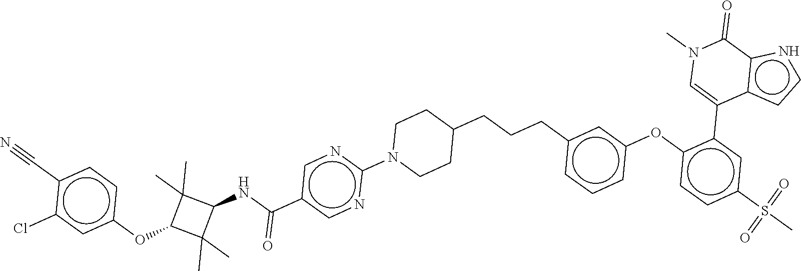 |
| I-250 | 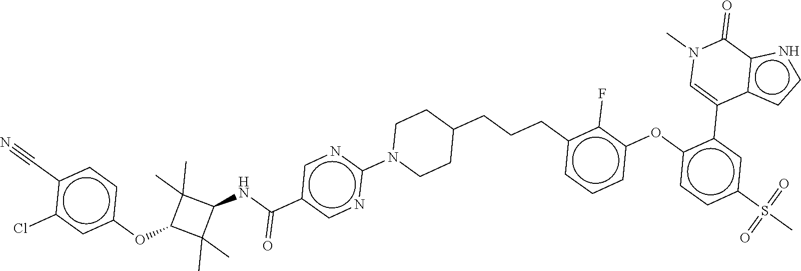 |
| I-251 | 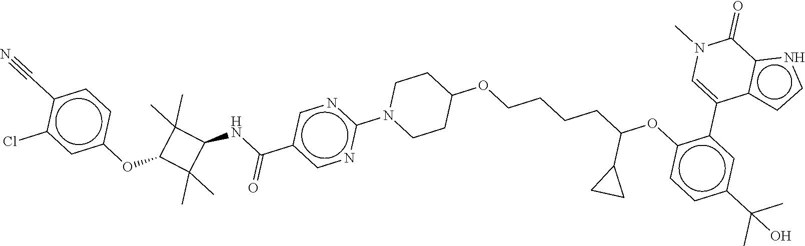 |
| I-252 | 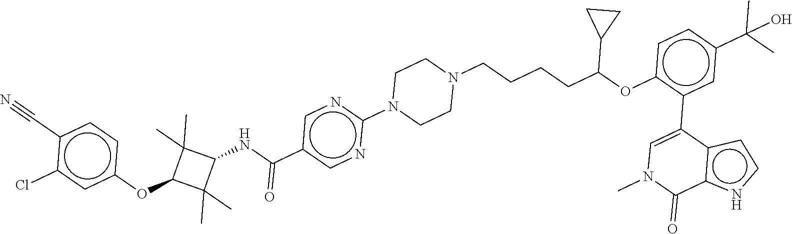 |
| I-253 | 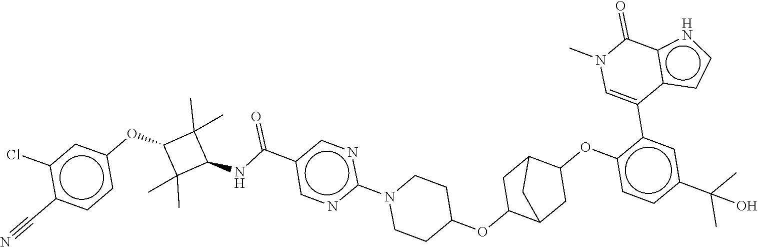 |
| I-254 | 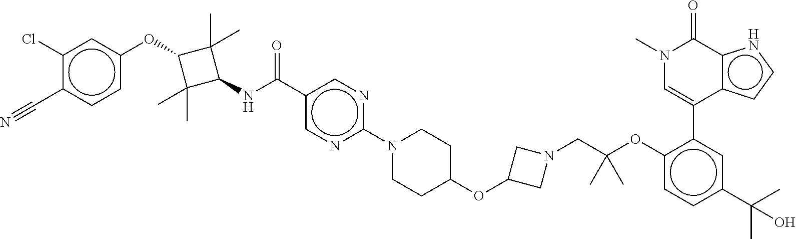 |
| I-255 | 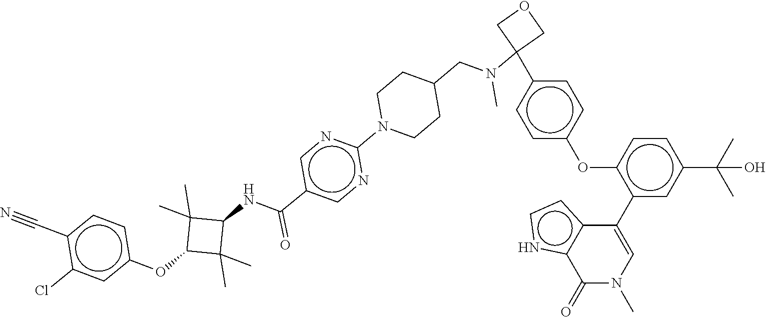 |
| I-256 | 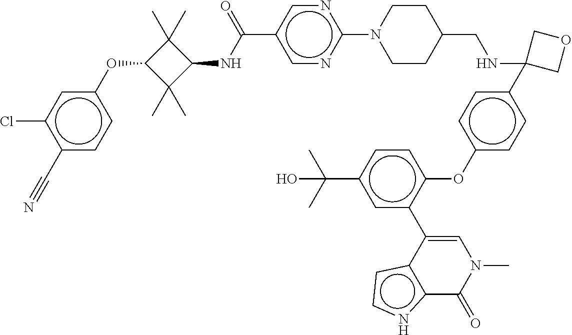 |
| I-257 | 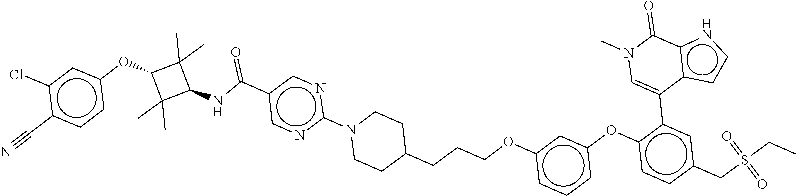 |
| I-258 | 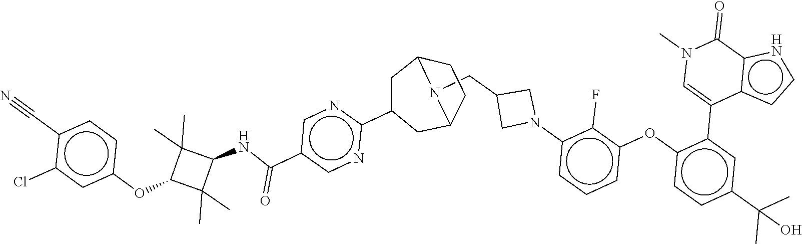 |
| I-259 | 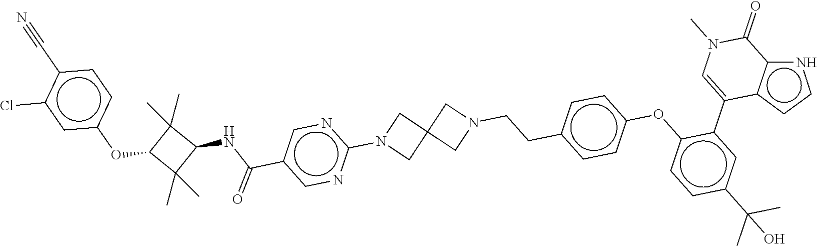 |
| I-260 | 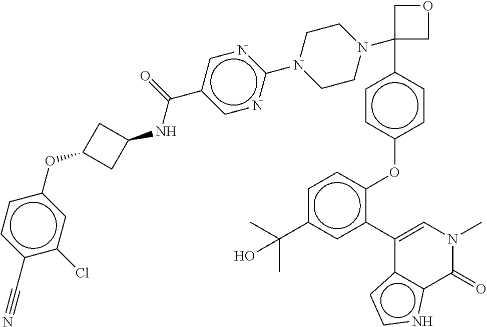 |
| I-261 | 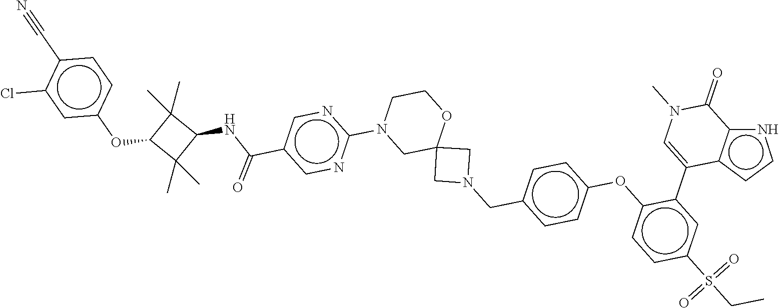 |
| I-262 | 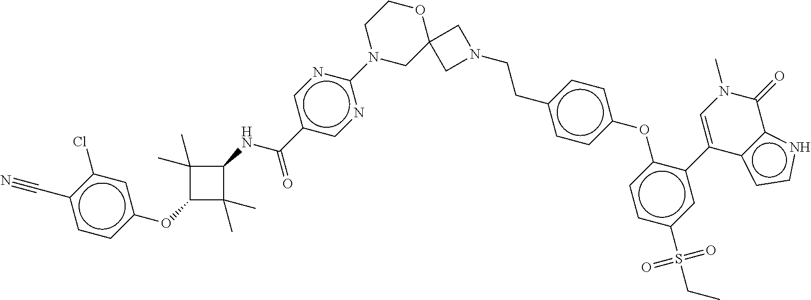 |
| I-263 | 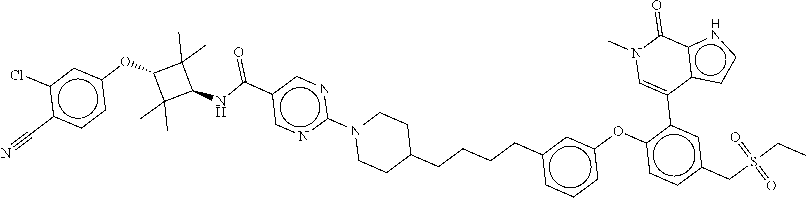 |
| I-264 | 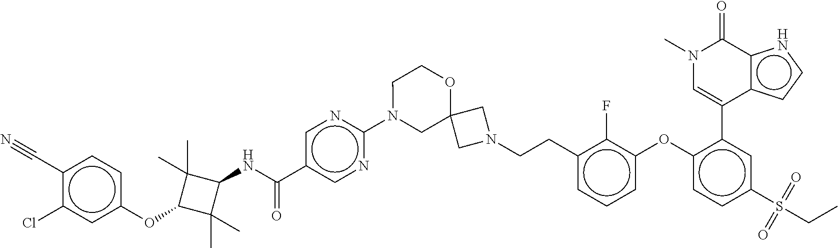 |
| I-265 | 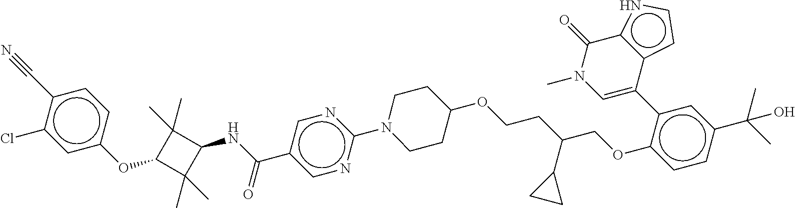 |
| I-266 | 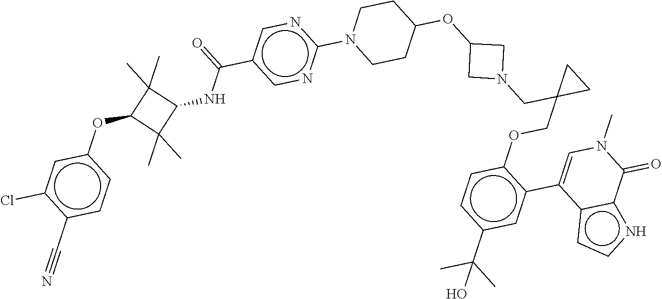 |
| I-267 | 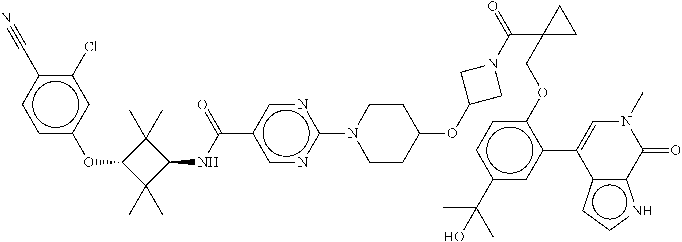 |
| I-268 | 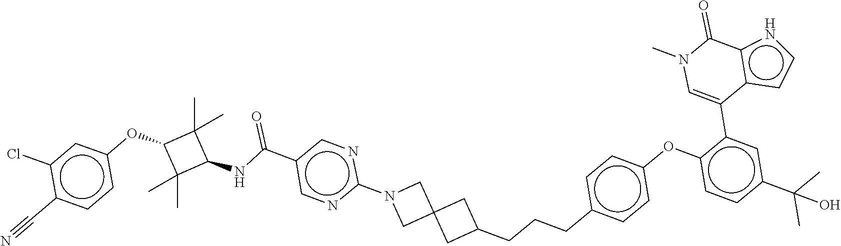 |
| I-269 | 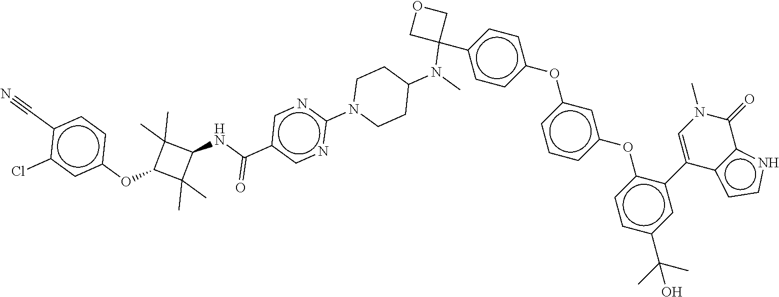 |
| I-270 | 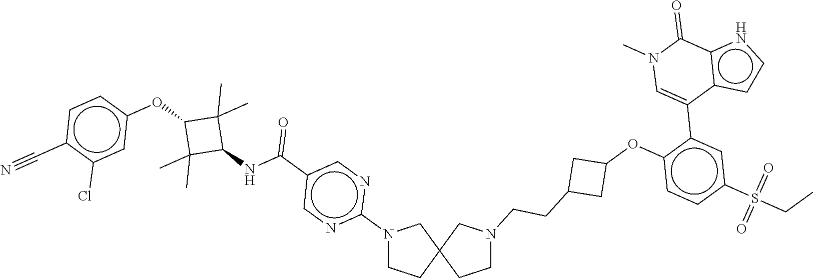 |
| I-271 | 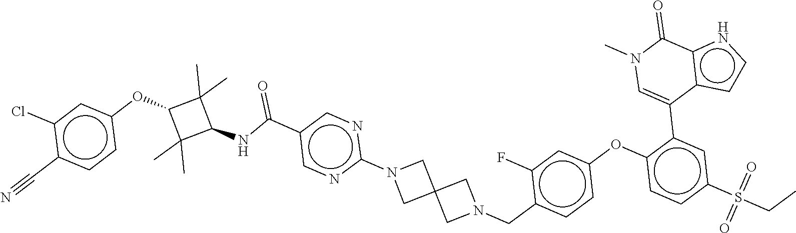 |
| I-272 | 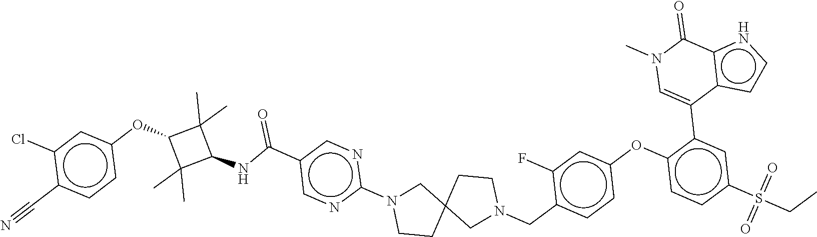 |
| I-273 | 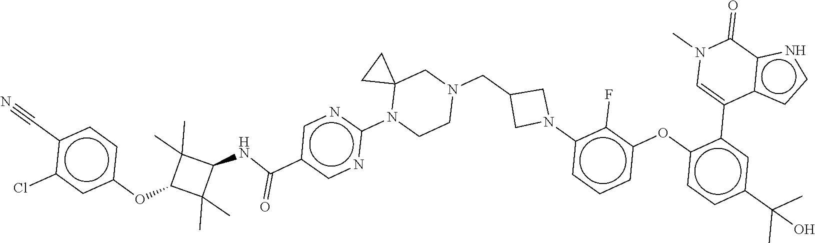 |
| I-274 | 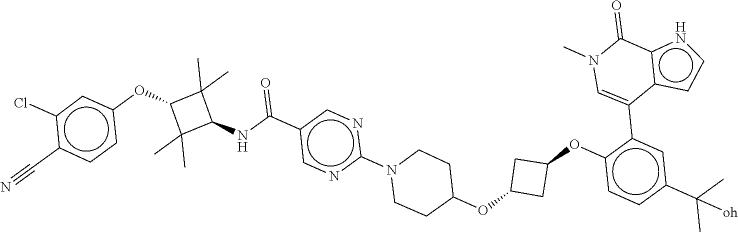 |
| I-275 | 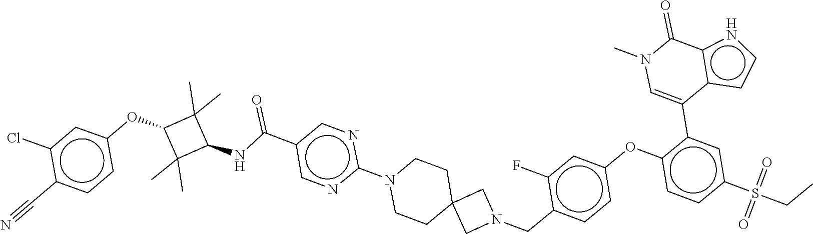 |
| I-276 | 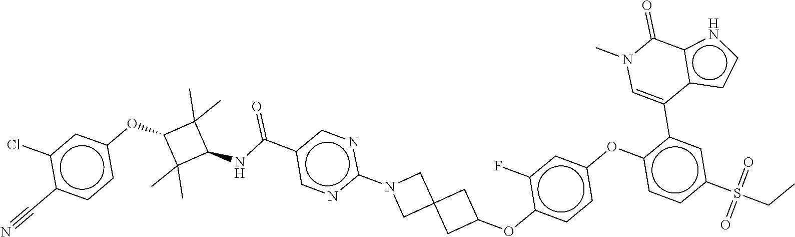 |
| I-277 | 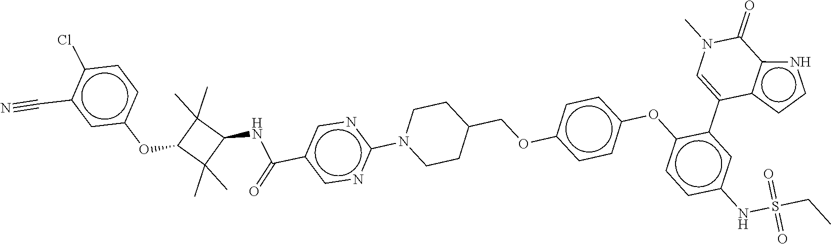 |
| I-278 | 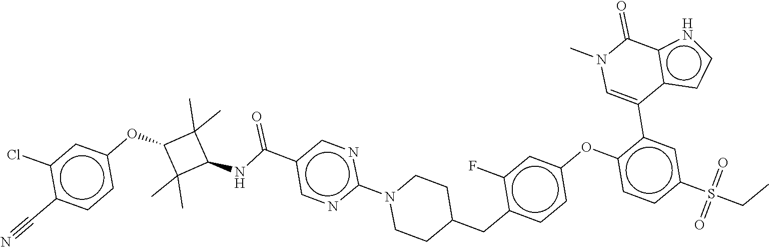 |
| I-279 | 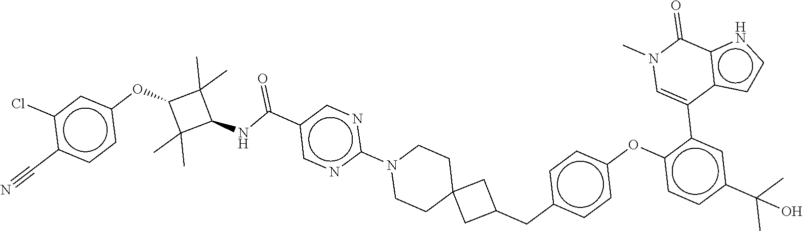 |
| I-280 | 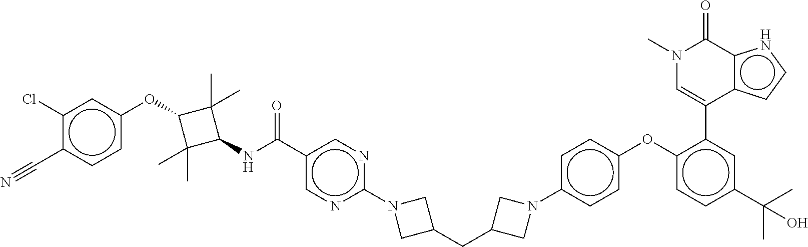 |
| I-281 | 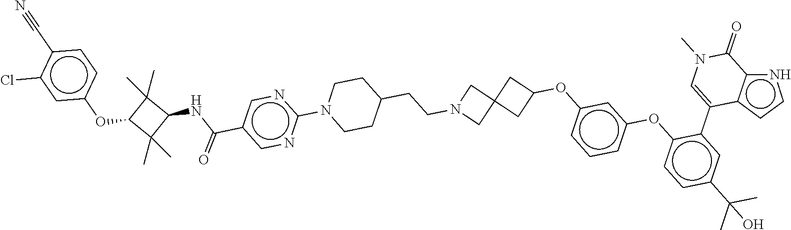 |
| I-282 | 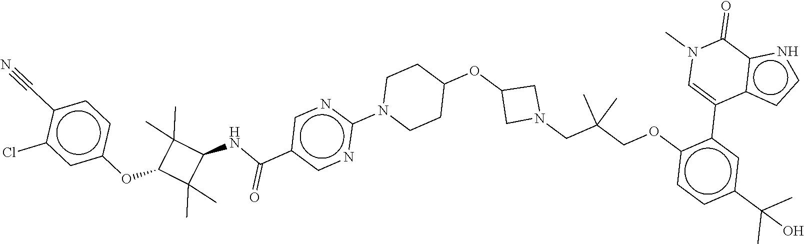 |
| I-283 | 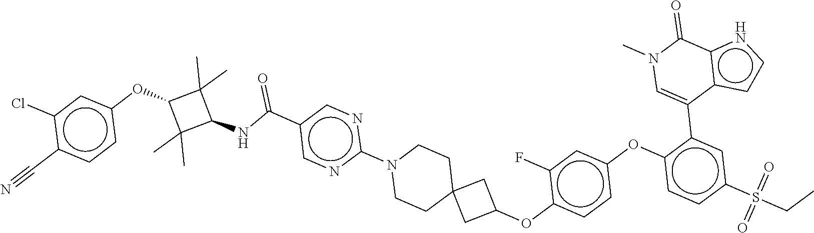 |
| I-284 | 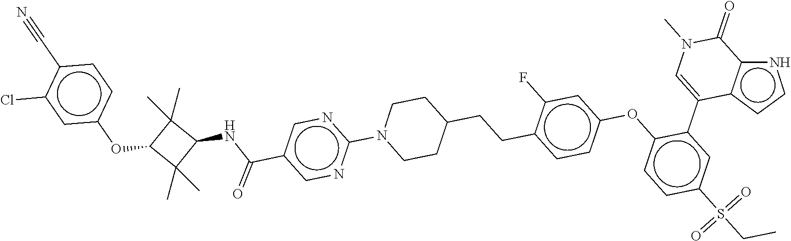 |
| I-285 | 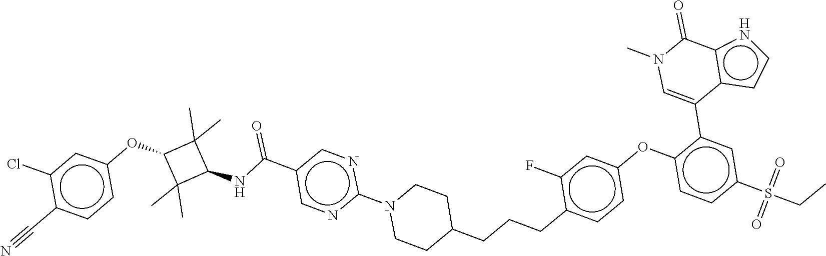 |
| I-286 | 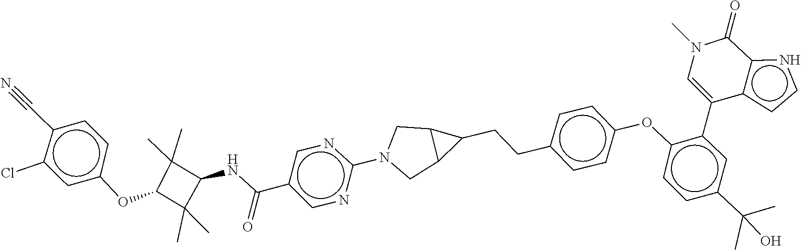 |
| I-287 | 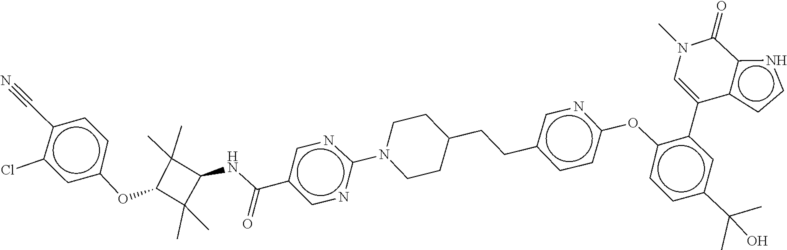 |
| I-288 | 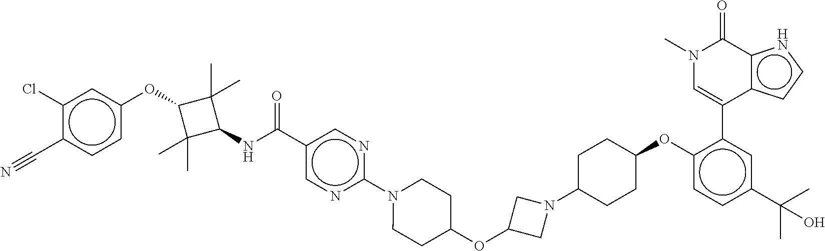 |
| I-289 | 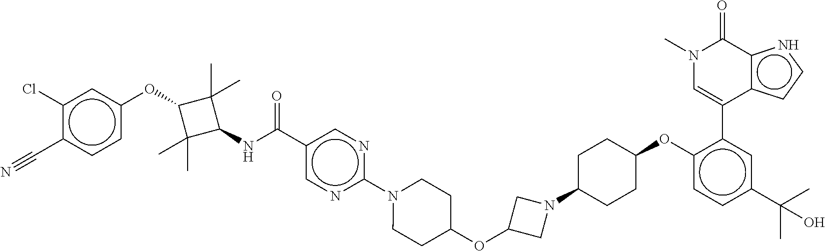 |
| I-290 | 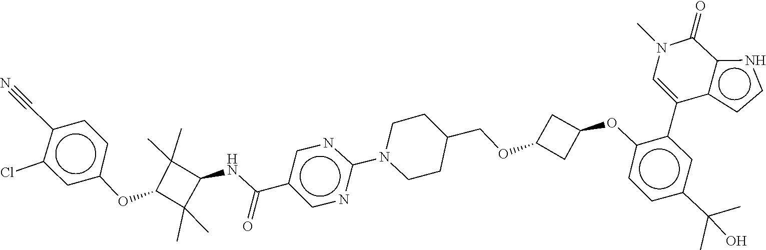 |
| I-291 | 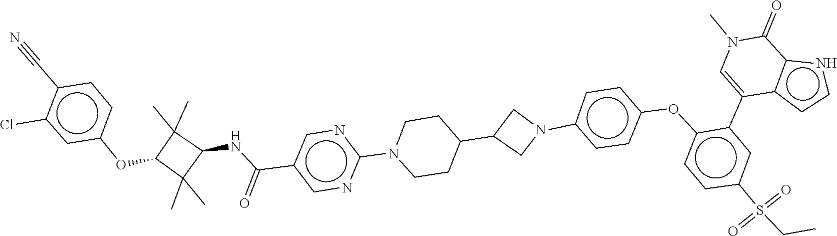 |
| I-292 | 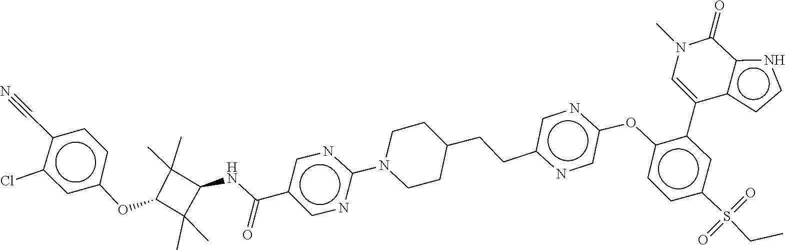 |
| I-293 | 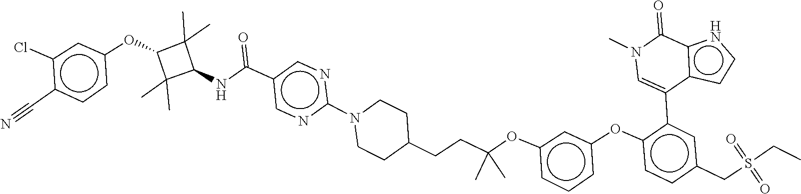 |
| I-294 | 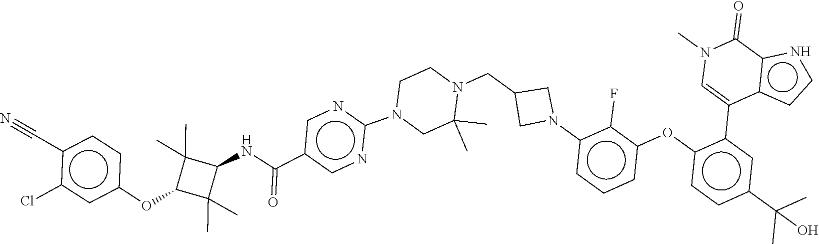 |
| I-295 | 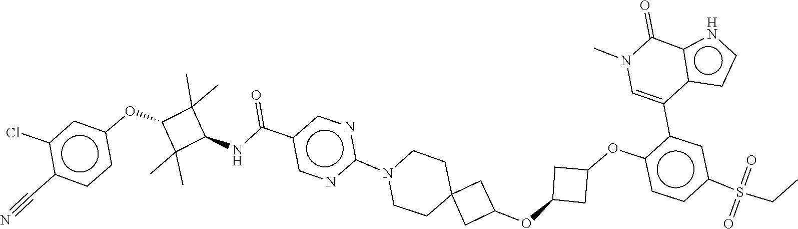 |
| I-296 | 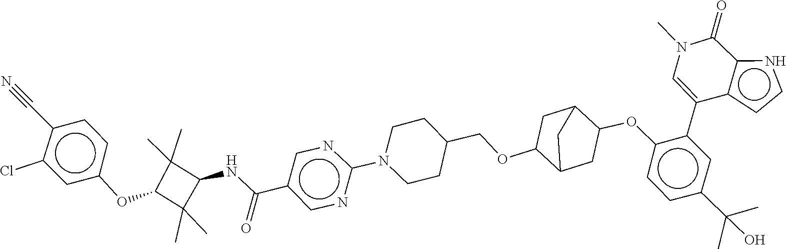 |
| I-297 | 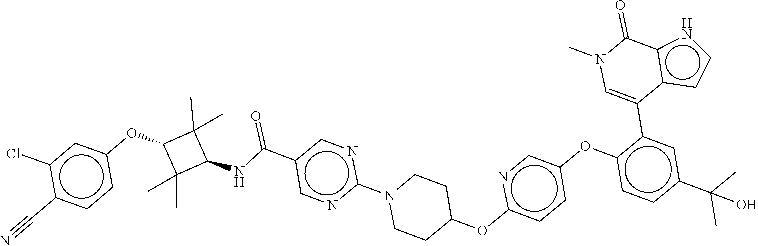 |
| I-298 | 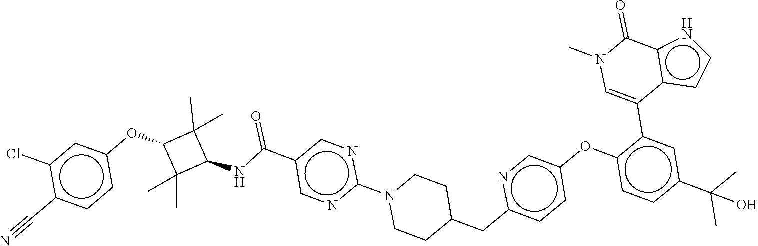 |
| I-299 | 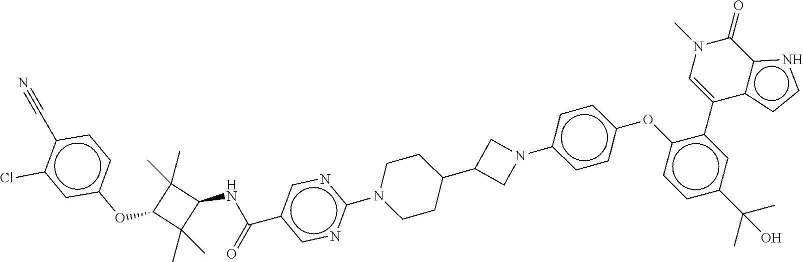 |
| I-300 | 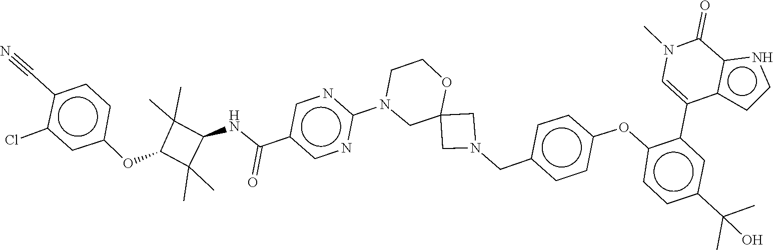 |
| I-301 | 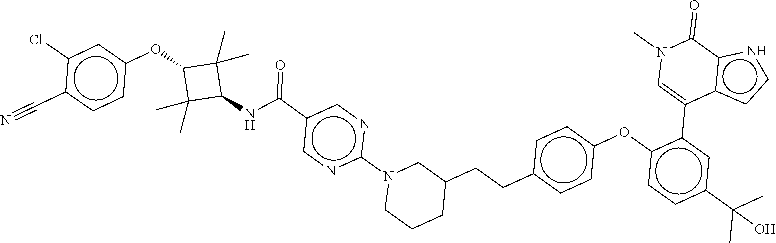 |
| I-302 | 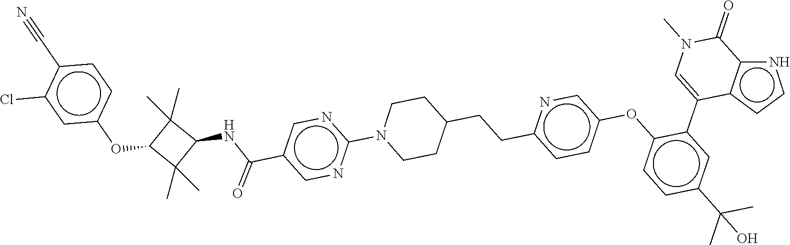 |
| I-303 | 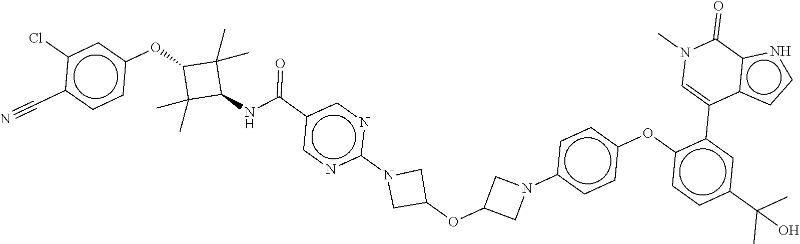 |
| I-304 | 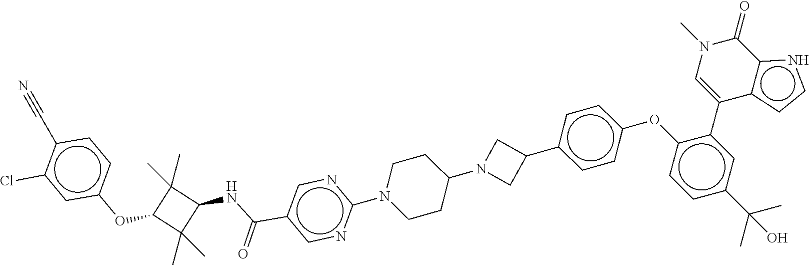 |
| I-305 | 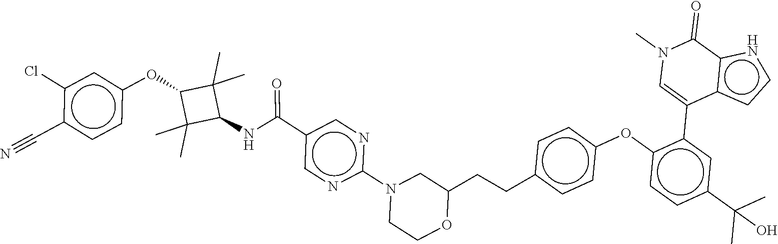 |
| I-306 | 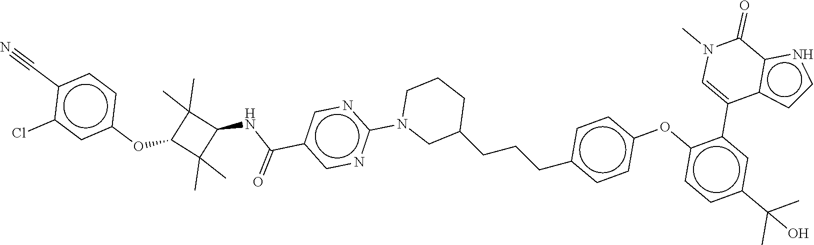 |
| I-307 | 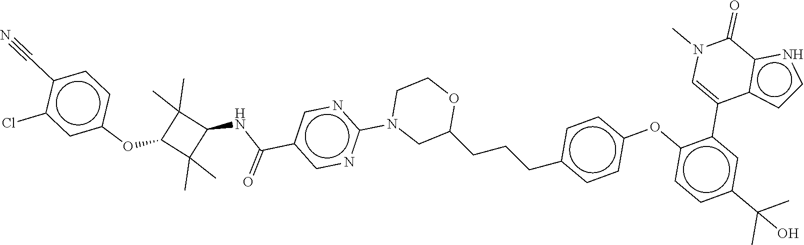 |
| I-308 | 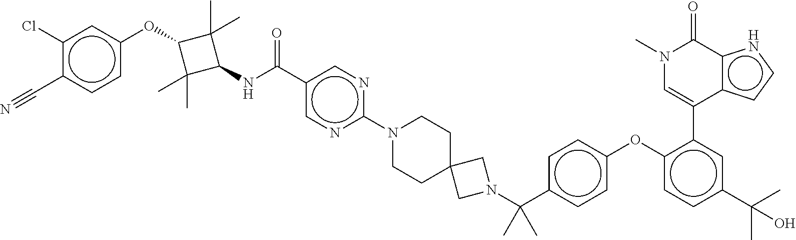 |
| I-309 | 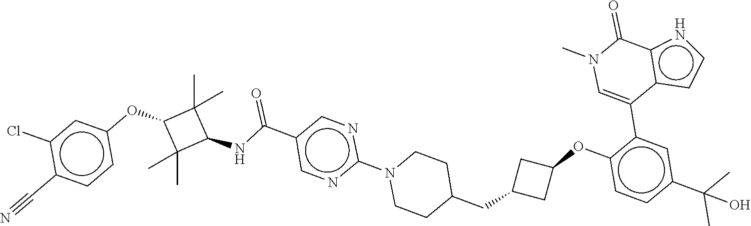 |
| I-310 | 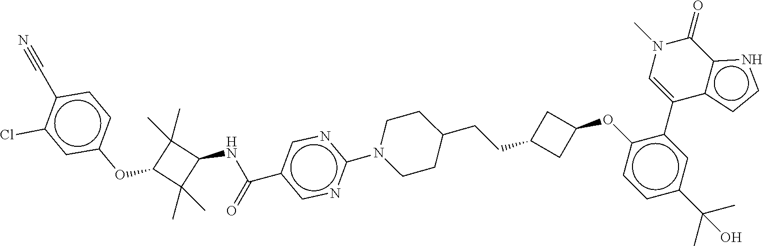 |
| I-311 | 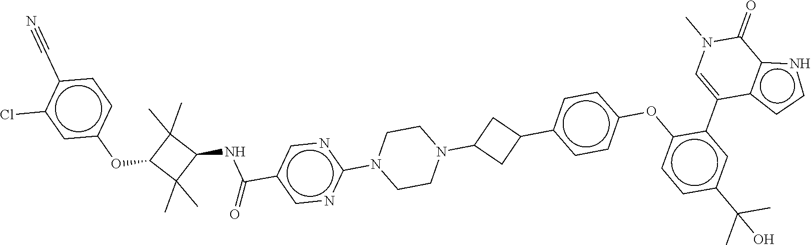 |
| I-312 | 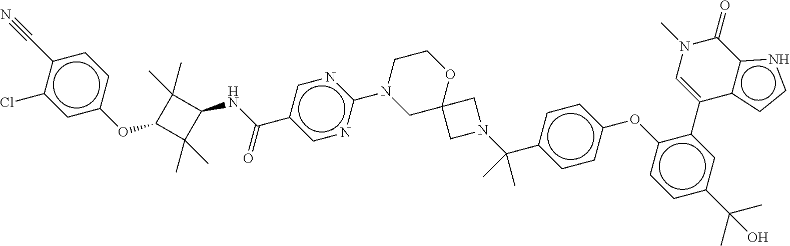 |
| I-313 | 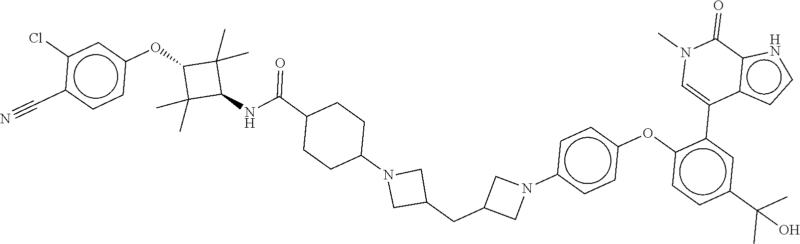 |
| I-314 | 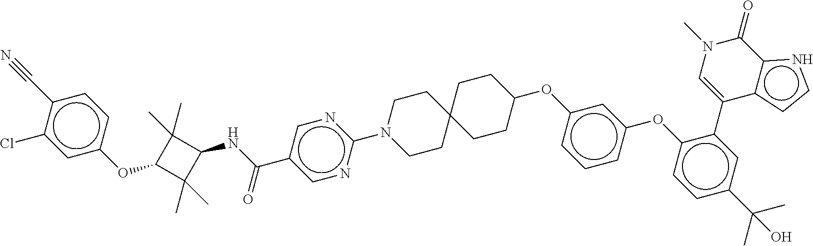 |
| I-315 | 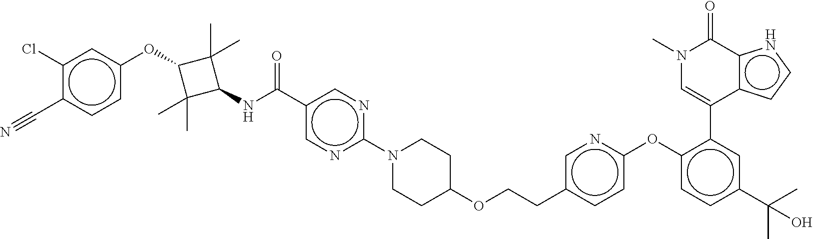 |
| I-316 | 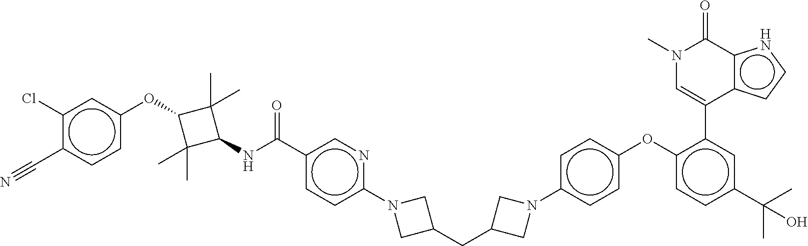 |
| I-317 | 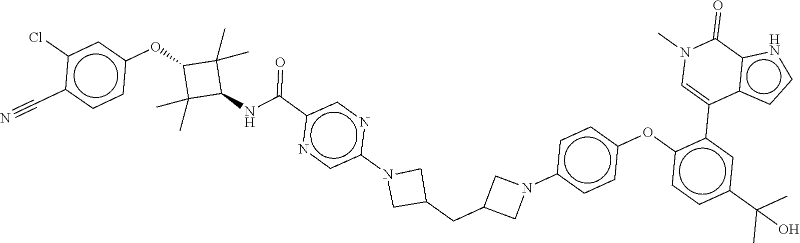 |
| I-318 | 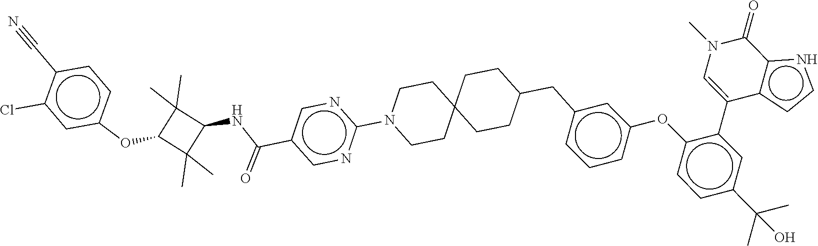 |
| I-319 | 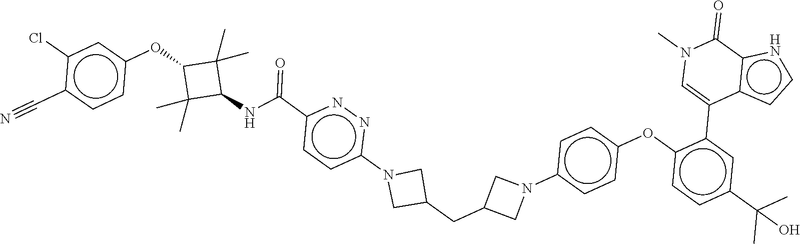 |
| I-320 | 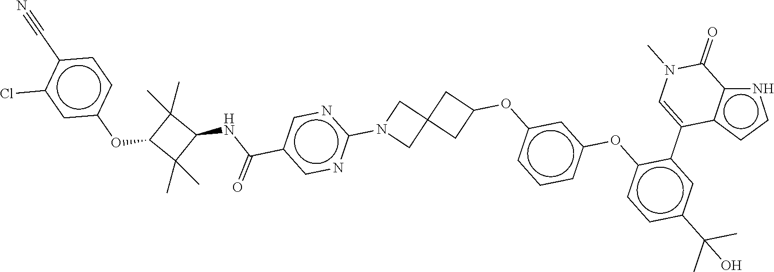 |
| I-321 | 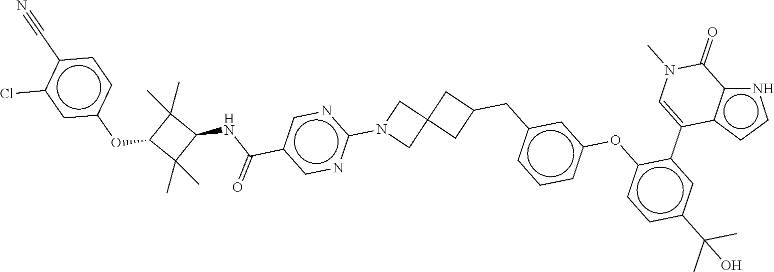 |
| I-322 | 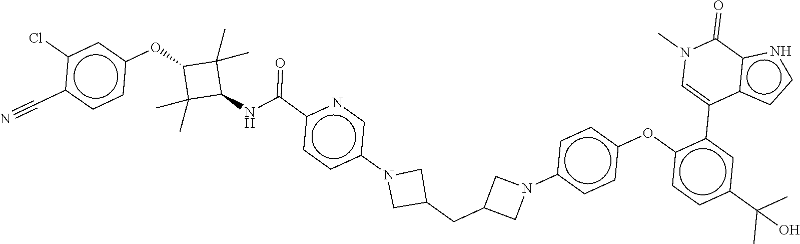 |
| I-323 | 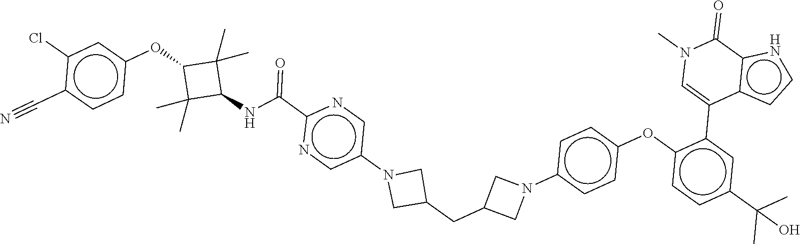 |
| I-324 | 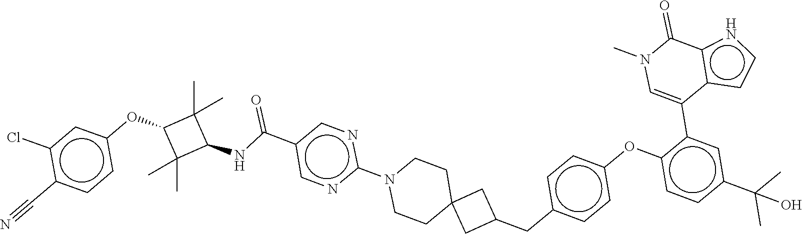 |
| I-325 | 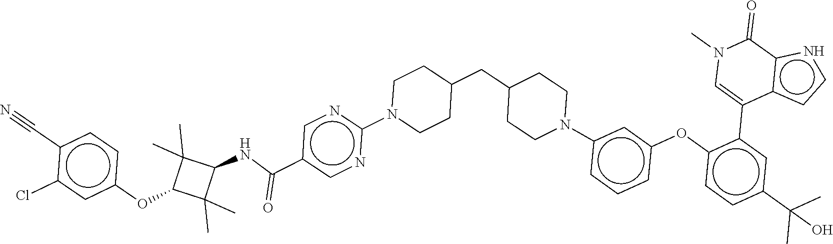 |
| I-326 | 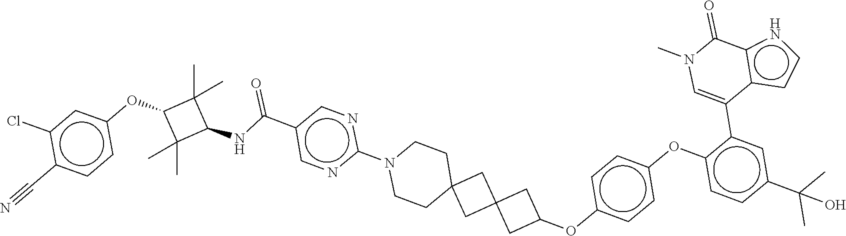 |
| I-327 | 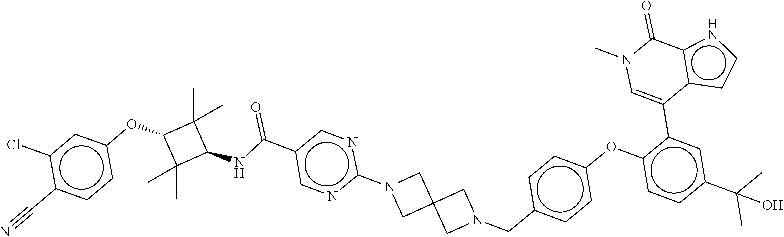 |
| I-328 | 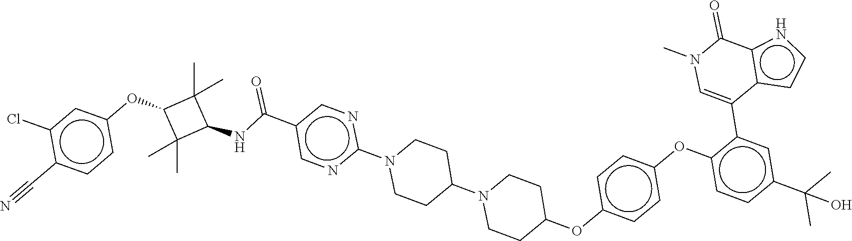 |
| I-329 | 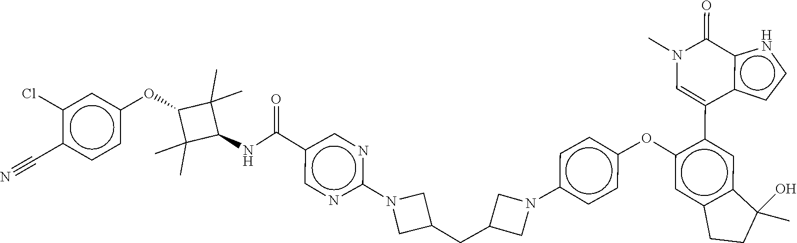 |
| I-330 | 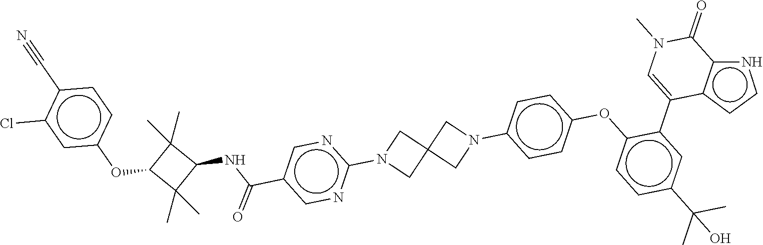 |
| I-331 | 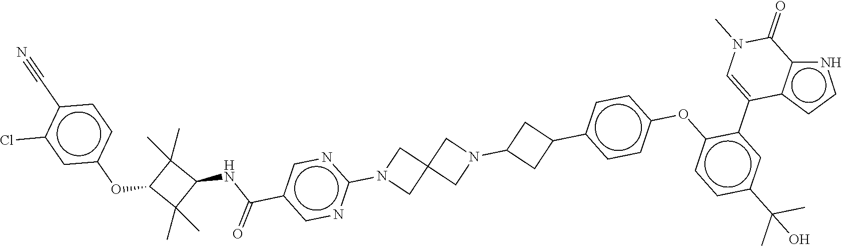 |
| I-332 | 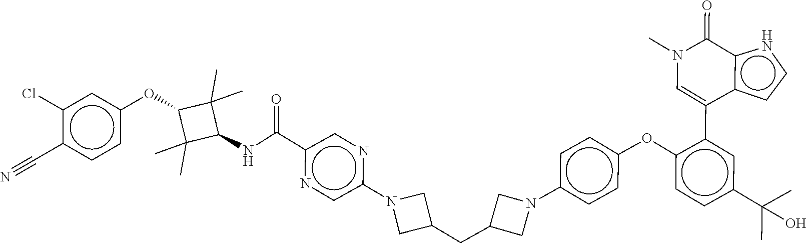 |
| I-333 | 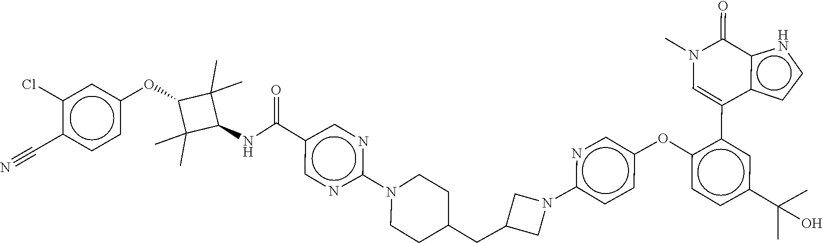 |
| I-334 | 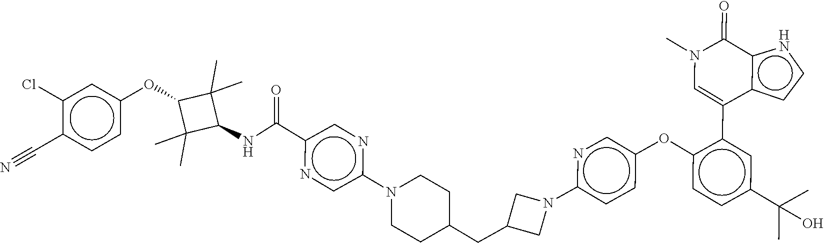 |
| I-335 | 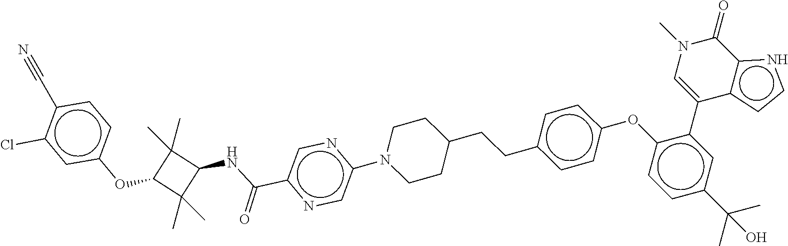 |
| I-336 | 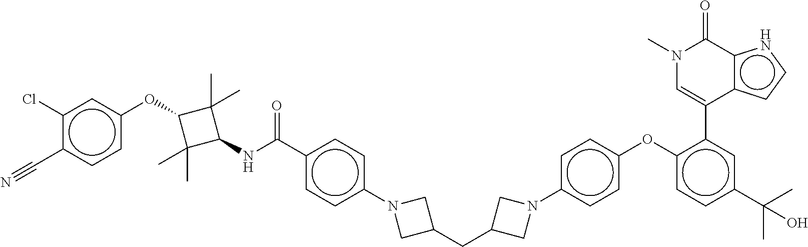 |
| I-337 | 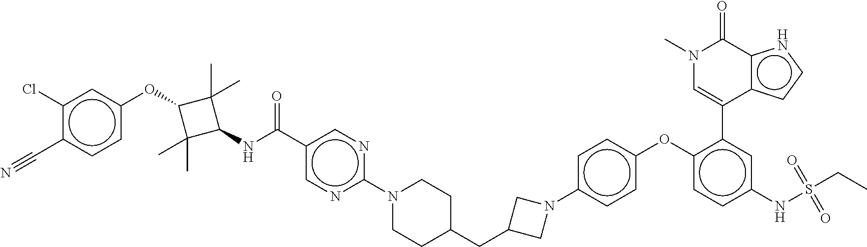 |
| I-338 | 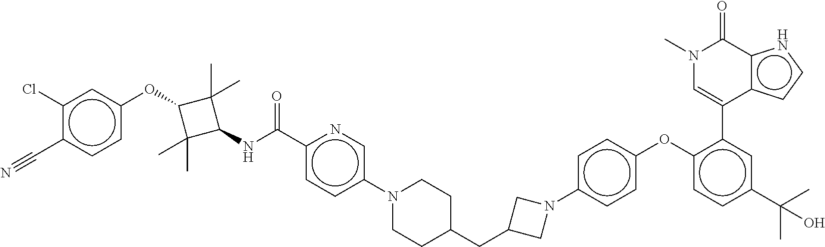 |
| I-339 | 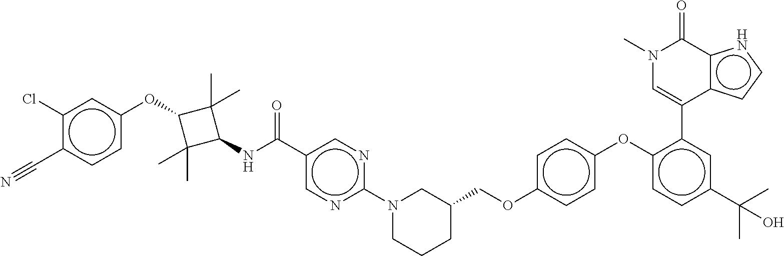 |
| I-340 | 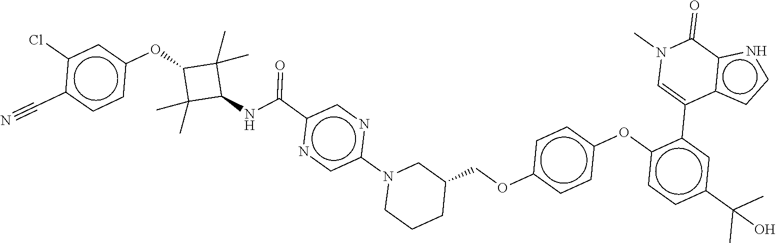 |
| I-341 | 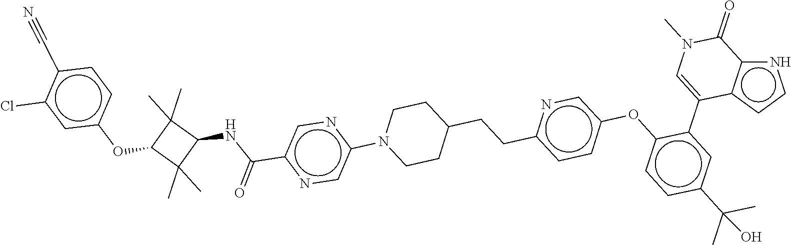 |
| I-342 | 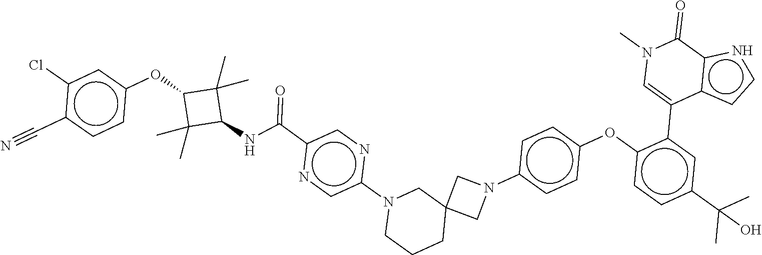 |
| I-343 | 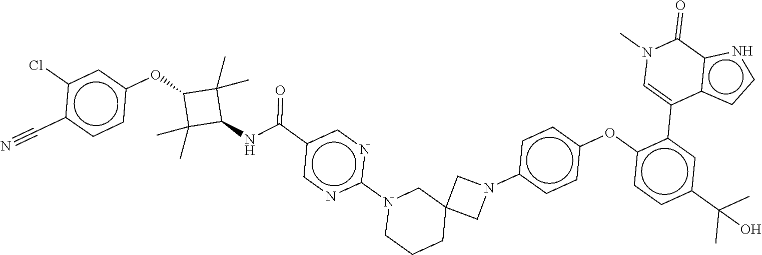 |
| I-344 | 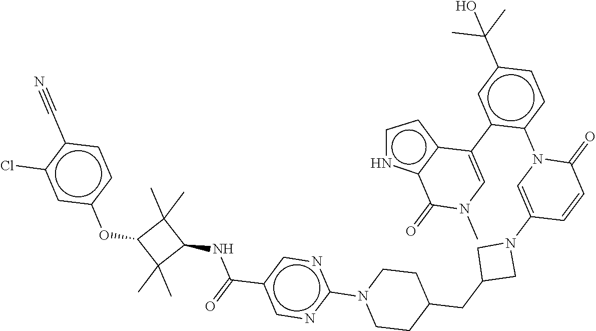 |
| I-345 | 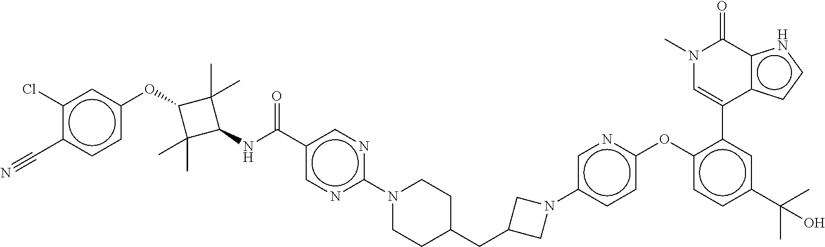 |
| I-346 | 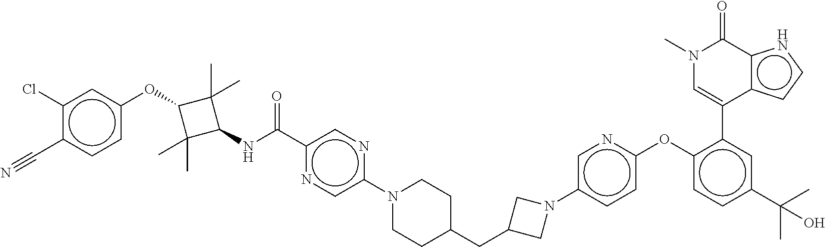 |
| I-347 | 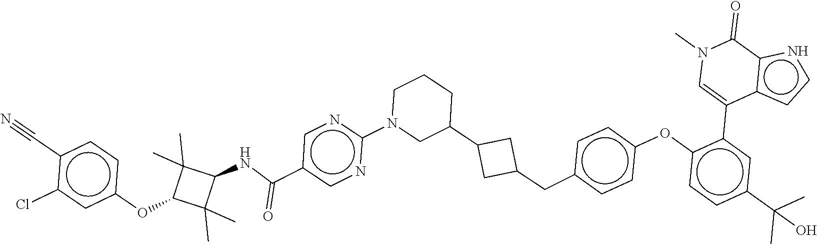 |
| I-348 | 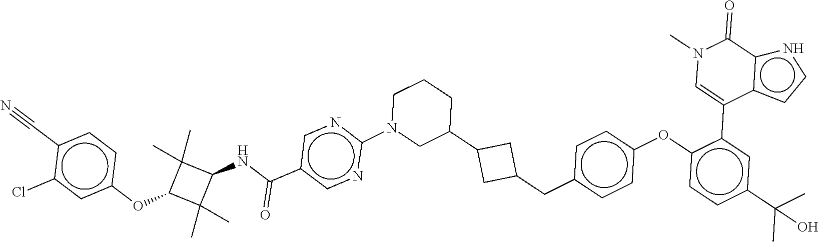 |
| I-349 | 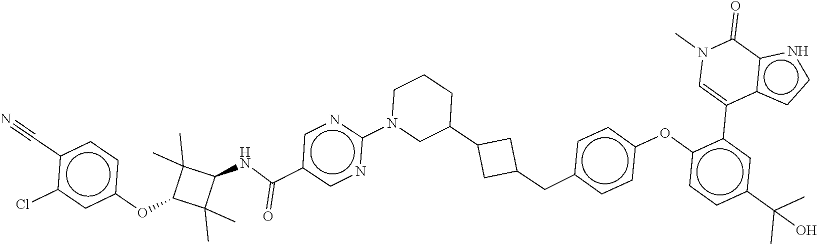 |
| I-350 | 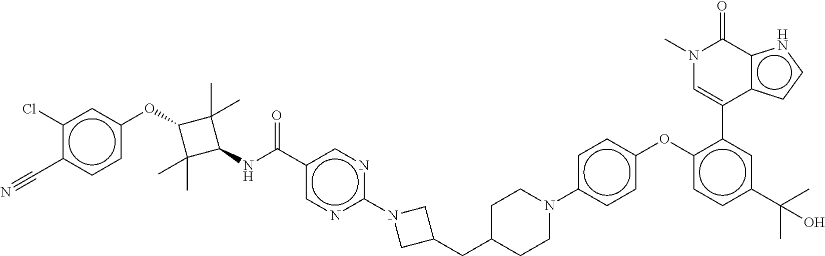 |
| I-351 | 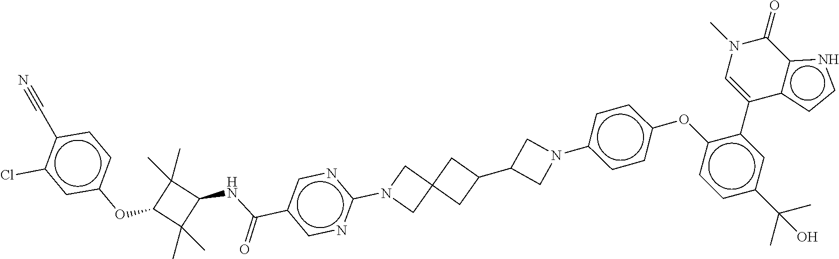 |
| I-352 | 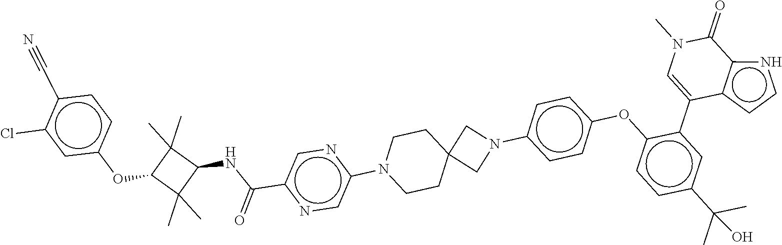 |
| I-353 | 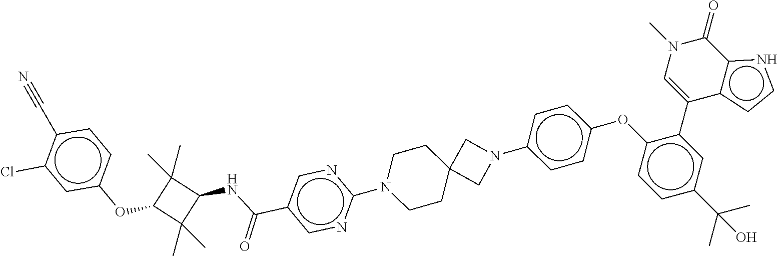 |
| I-354 | 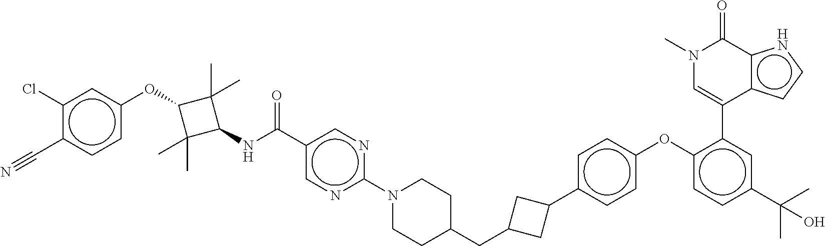 |
| I-355 | 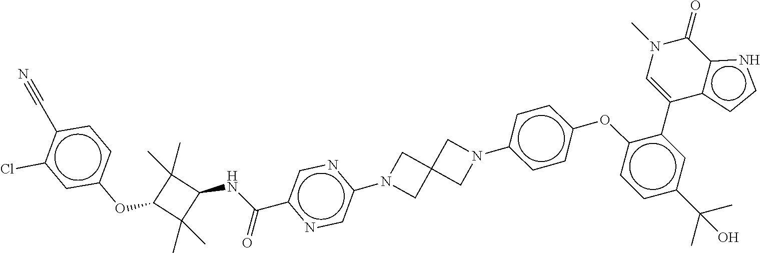 |
| I-356 | 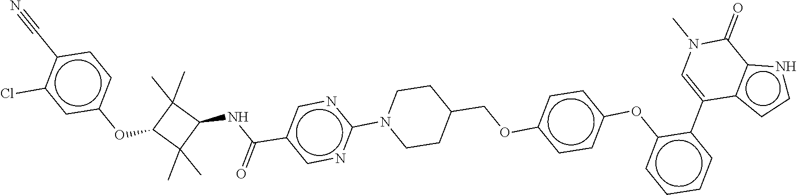 |
| I-357 | 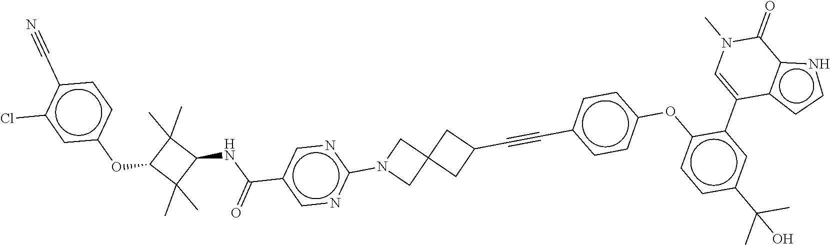 |
| I-358 | 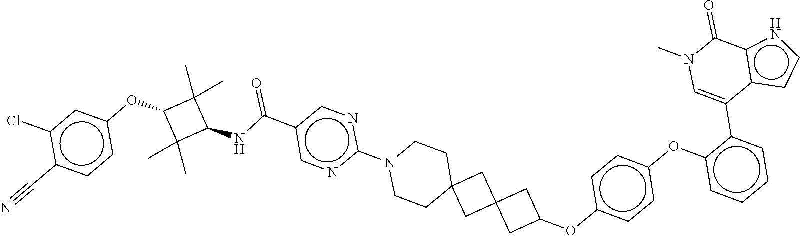 |
| I-359 | 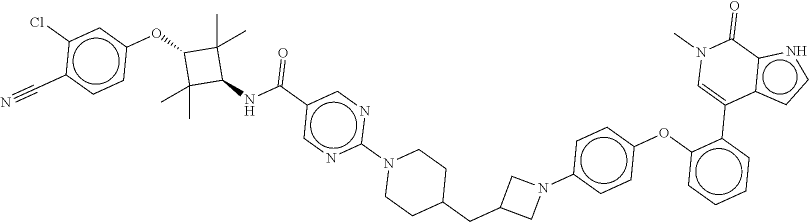 |
| I-360 | 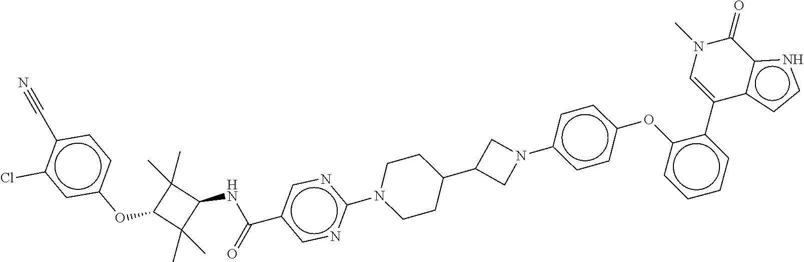 |
| I-361 | 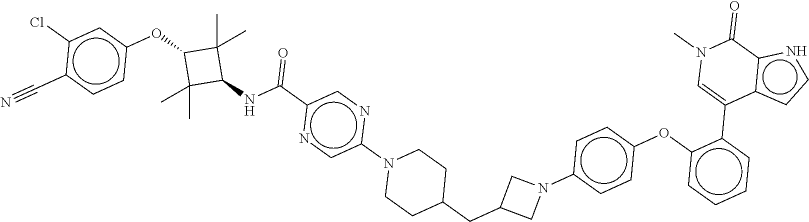 |
| I-362 | 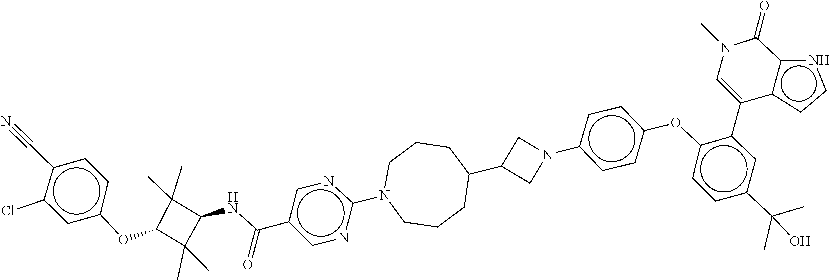 |
| I-363 | 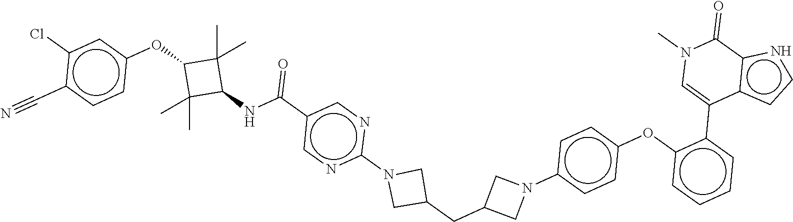 |
| I-364 | 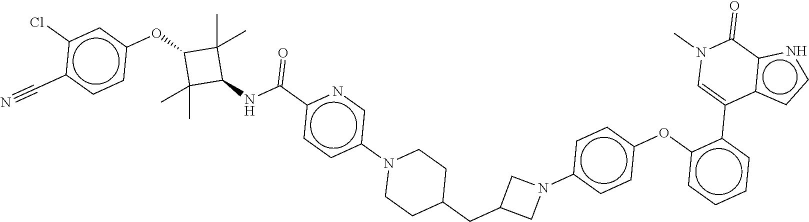 |
| I-365 | 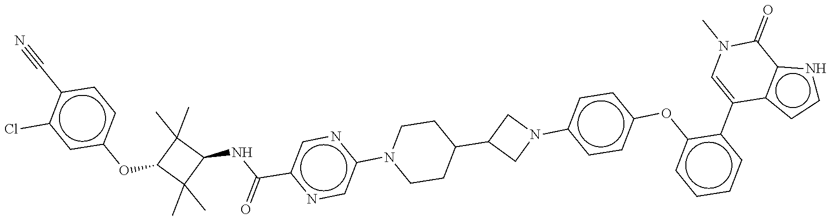 |
| I-366 | 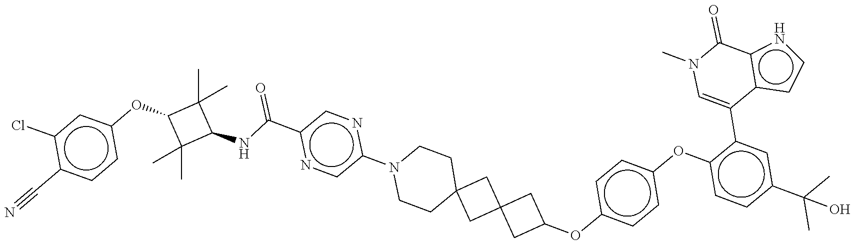 |
| I-367 | 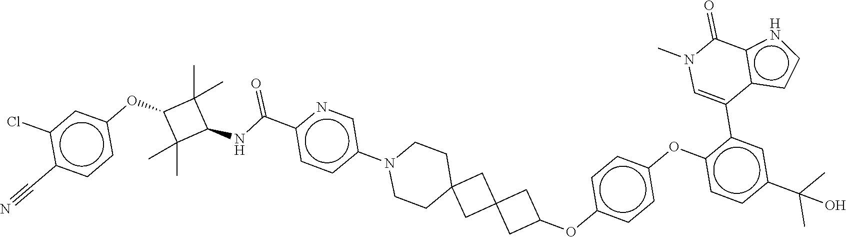 |
| I-368 | 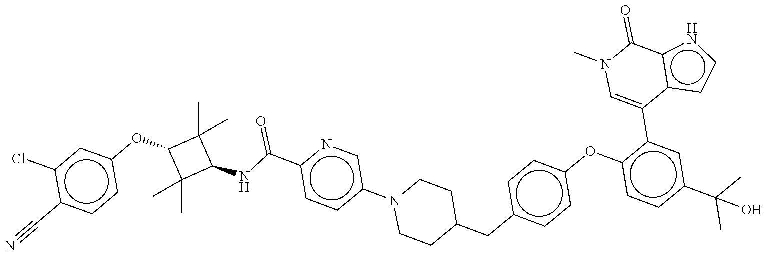 |
| I-369 | 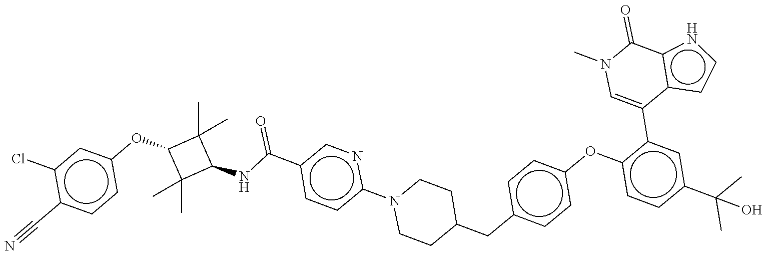 |
| I-370 | 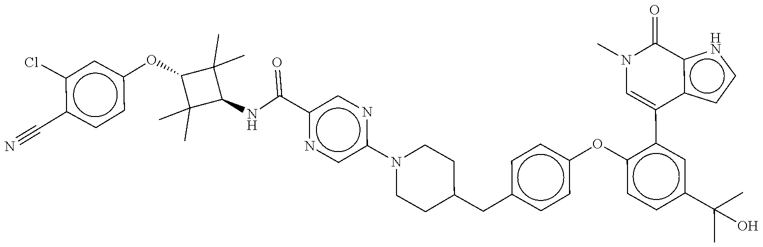 |
| I-371 | 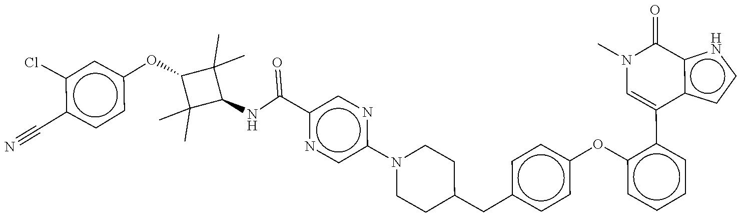 |
| I-372 | 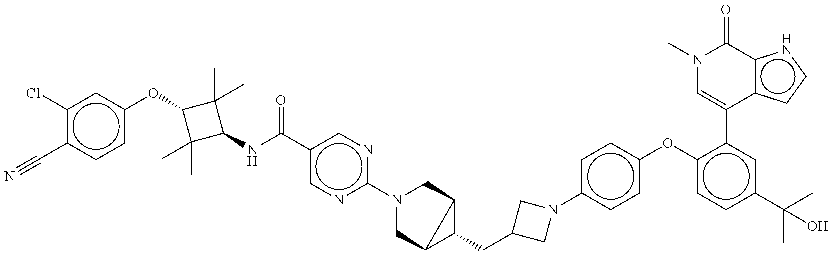 |
| I-373 | 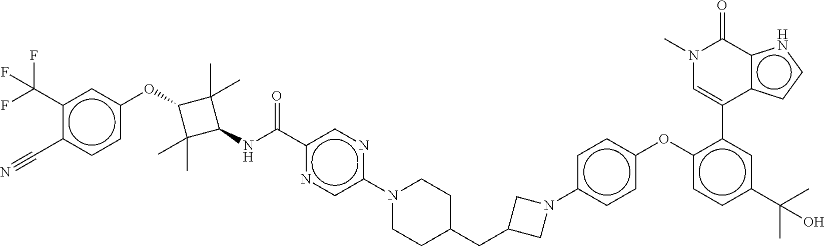 |
| I-374 | 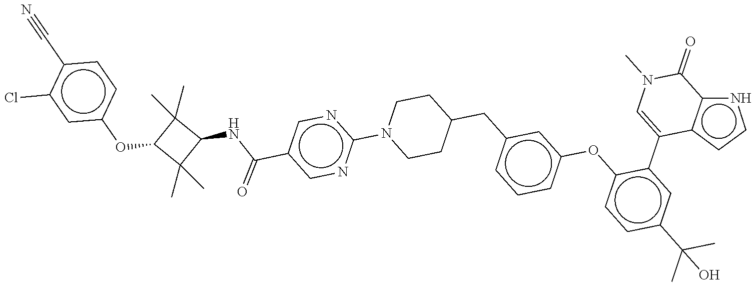 |
| I-375 | 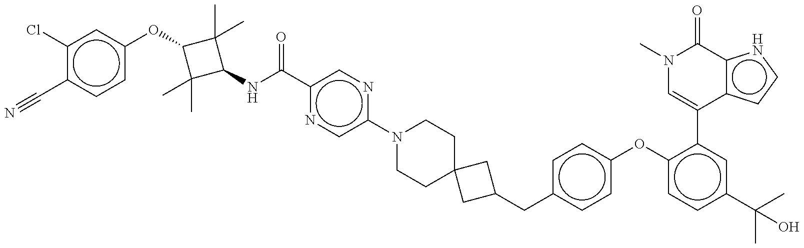 |
| I-376 | 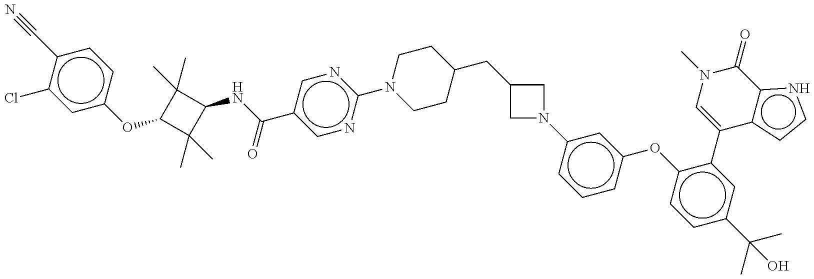 |
| I-377 | 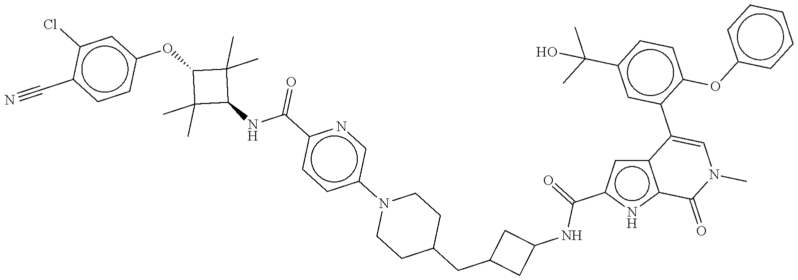 |
| I-378 | 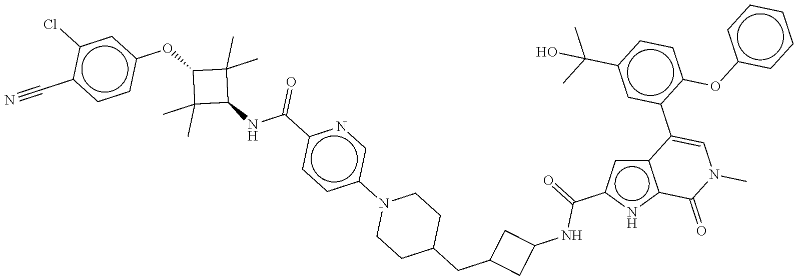 |
| I-379 | 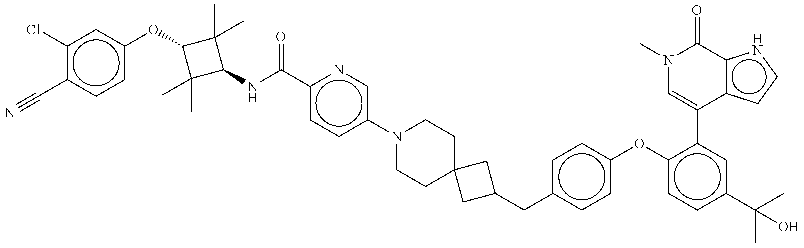 |
| I-380 | 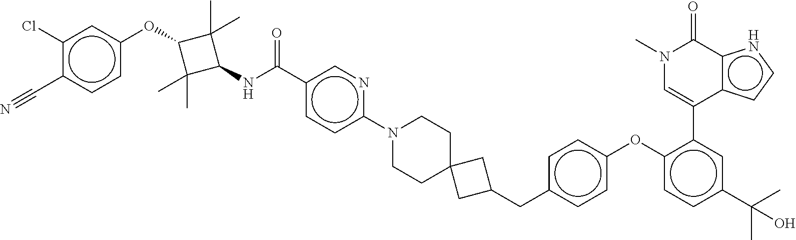 |
| I-381 | 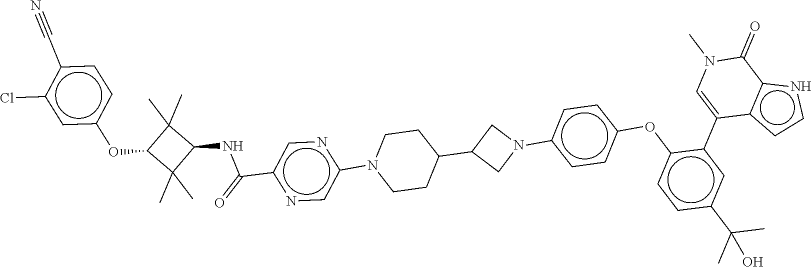 |
| I-382 | 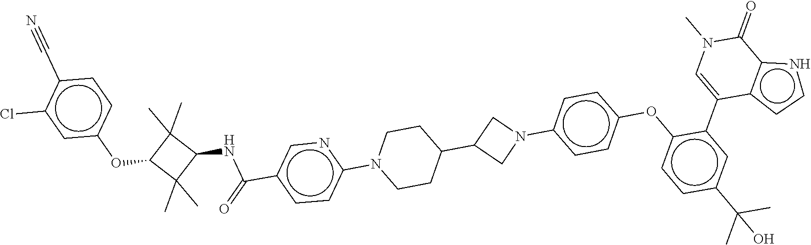 |
| I-383 | 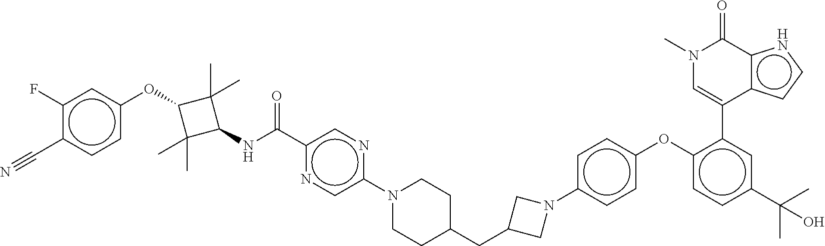 |
| I-384 | 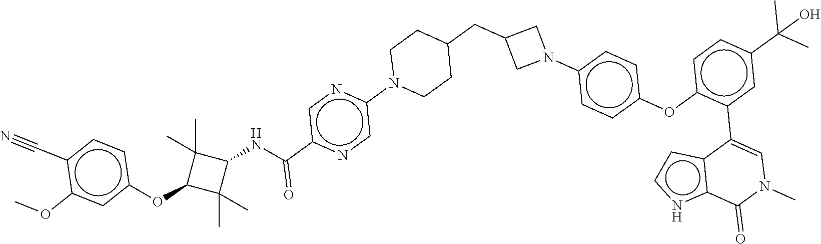 |
| I-385 | 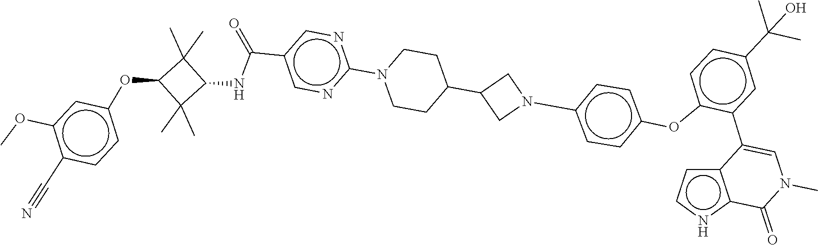 |
| I-386 | 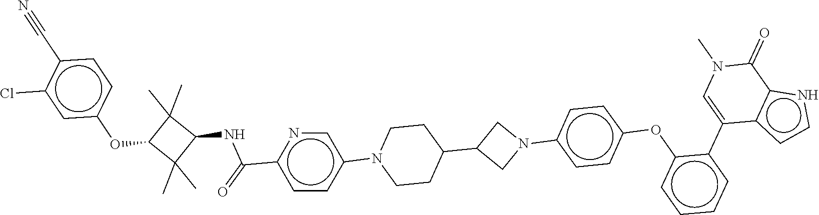 |
| I-387 | 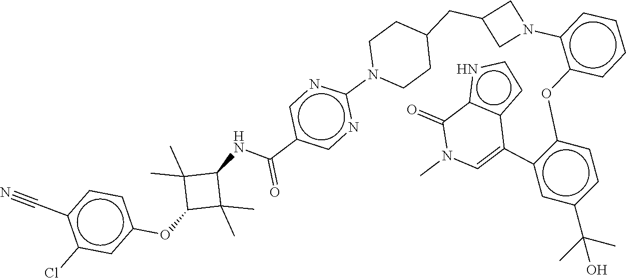 |
| I-388 | 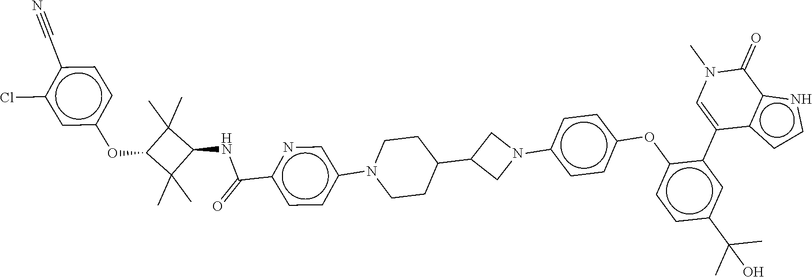 |
| I-389 | 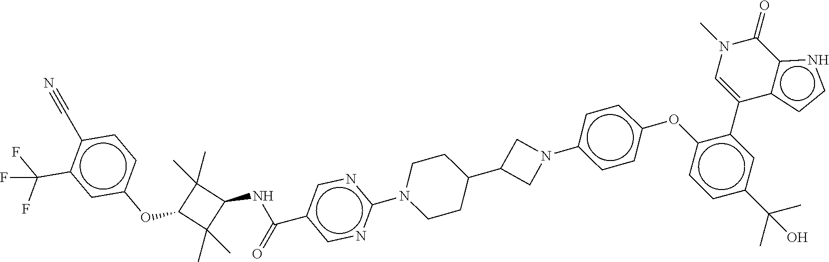 |
| I-390 | 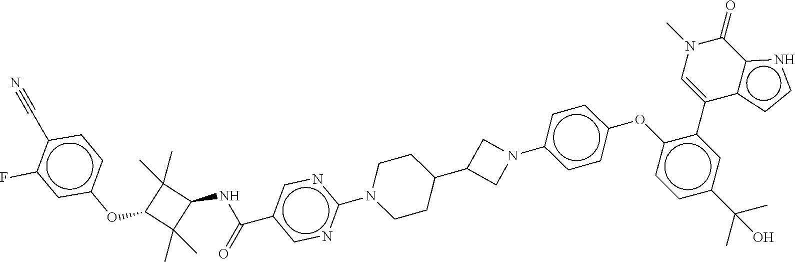 |
| I-391 | 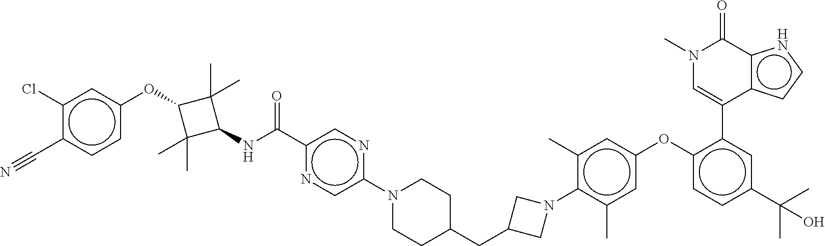 |
| I-392 | 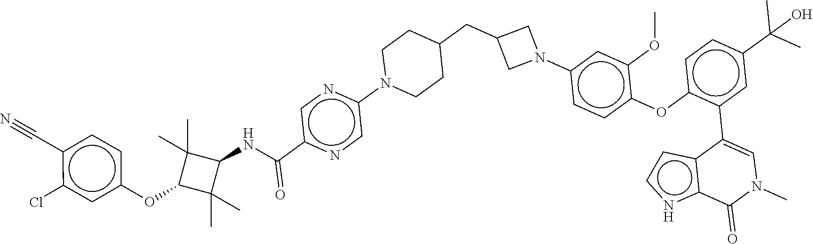 |
| I-393 | 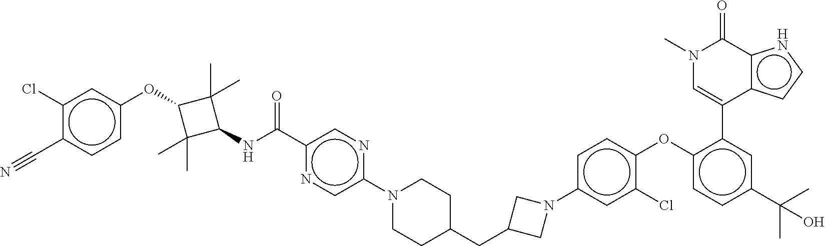 |
| I-394 | 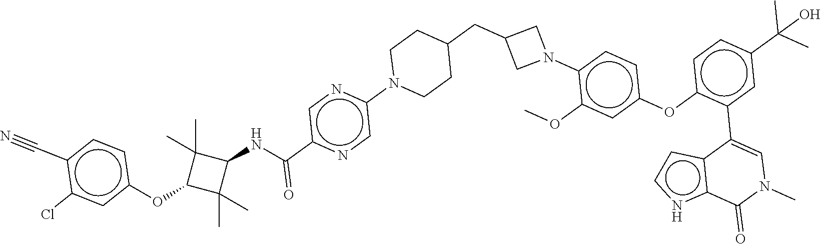 |

### Synthetic Methods

Methods for preparing compounds described herein are illustrated in the following synthetic Schemes. The Schemes are given for the purpose of illustrating the invention, and are not intended to limit the scope or spirit of the invention. Starting materials shown in the Schemes can be obtained from commercial sources or can be prepared based on procedures described in the literature.

In the Schemes, it is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated (for example, use of protecting groups or alternative reactions). Protecting group chemistry and strategy is well known, such as described in, for example, “Protecting Groups in Organic Synthesis”, T. W. Greene and P. G. M. Wuts, 3^{rd} edition, John Wiley & Sons, 1999, the entire contents of which are hereby incorporated by reference.

The synthetic route illustrated in Scheme 1 is a general method for preparing compounds of Formula H. Coupling fluoro-nitrobenzene compound A with substituted phenol B provides phenyl ether compound C. Reduction of the nitro group in compound C provides substituted aniline D. Reaction of substituted aniline D with ClSO₂R provides sulfonamide E. Removal of the protection group (Pg) from compound E provides compound F. The Pg may be, for example, a Boc protecting group that can be removed by treating the compound with trifluoroacetic acid. Coupling compound F with compound G (such as a nucleophilic aromatic substitution reaction when X is an amino group and the leaving group in compound E is chloro) provides the final compound of Formula H.

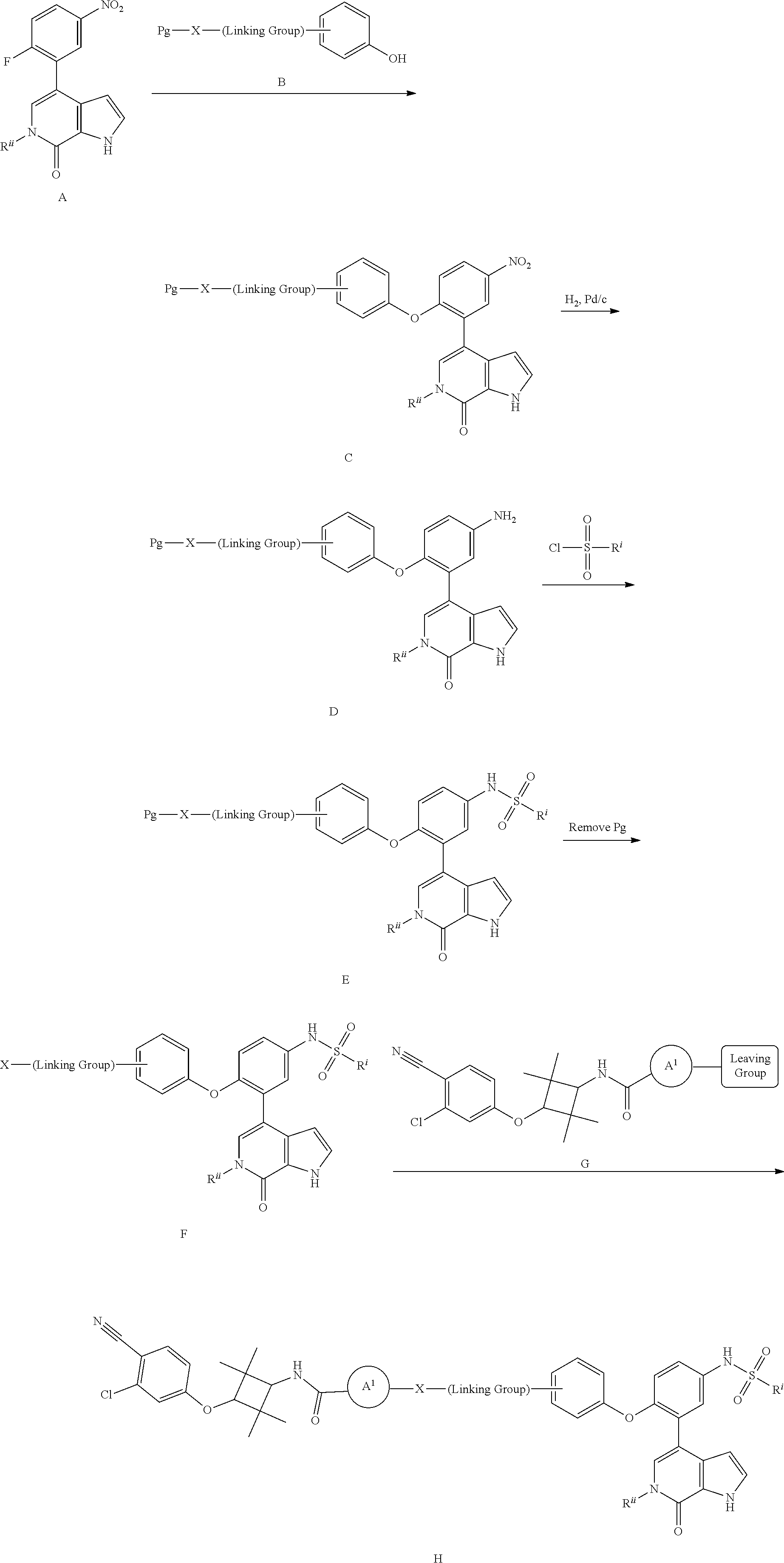

The modular synthetic route illustrated in Scheme 1 can be readily modified to provide additional compounds by conducting functional group transformations on the intermediate and/or final compounds. Such functional group transformations are well known in the art, as described in, for example, Comprehensive Organic Synthesis (B. M. Trost & I. Fleming, eds., 1991-1992); Organic Synthesis, 3^{rd} Ed (Michael B. Smith, Wavefunction, Inc., Irvine: 2010); Modern Methods of Organic Synthesis, 4^{th} Ed. (William Carruthers and lain Coldham, Cambridge University Press, Cambridge: 2004); March's Advanced Organic Chemistry: Reactions, Mechanisms, and Structure, 8^{th} Ed., (Michael B. Smith, John Wiley & Sons, New York: 2020); and Comprehensive Organic Transformations: A Guide to Functional Group Preparations, 3rd Ed (Richard C. Larock, ed., John Wiley & Sons, New York: 2018). Protecting group strategies may be deployed as appropriate to accommodate differing functional groups in the molecules used in the synthetic route. Protecting group chemistry and strategy is described in, for example, Protecting Groups in Organic Synthesis, 3^{rd} Edition, T. W. Greene and P. G. M. Wuts, John Wiley & Sons, 1999 and Greene's Protective Groups in Organic Synthesis, 5th Ed., (Peter G. M. Wuts, John Wiley & Sons: 2014).

### II. Therapeutic Applications

The heterobifunctional compounds described herein, such as a compound of Formula I, II, or other compounds in Section I, provide therapeutic benefits to patients suffering from cancer. Accordingly, one aspect of the invention provides a method of treating cancer. The method comprises administering to a patient in need thereof a therapeutically effective amount of a compound described herein, such as a compound of Formula I, II, or other compound in Section I, to treat the cancer. In certain embodiments, the compound is a compound of Formula I. In certain embodiments, the particular compound of Formula I is a compound defined by one of the embodiments described above.

### Cancer

In certain embodiments, the cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct and gallbladder cancers, liver cancer, pancreatic cancer, bladder cancer, urinary tract cancer, kidney cancer, eye cancer, thyroid cancer, lymphoma, or leukemia. In certain embodiments, the cancer is prostate cancer.

In certain embodiments, the cancer is squamous cell cancer, lung cancer including small cell lung cancer, non-small cell lung cancer, vulval cancer, thyroid cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, hepatic carcinoma, anal carcinoma, penile carcinoma, and head and neck cancer. In certain embodiments, the cancer is at least one selected from the group consisting of ALL, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL, Philadelphia chromosome positive CML, lymphoma, leukemia, multiple myeloma myeloproliferative diseases, large B cell lymphoma, or B cell Lymphoma.

In certain embodiments, the cancer is a solid tumor or leukemia. In certain other embodiments, the cancer is colon cancer, pancreatic cancer, breast cancer, ovarian cancer, prostate cancer, squamous cell carcinoma, basal cell carcinoma, adenocarcinoma, sweat gland carcinoma, sebaceous gland carcinoma, lung cancer, leukemia, bladder cancer, stomach cancer, cervical cancer, testicular cancer, skin cancer, rectal cancer, thyroid cancer, kidney cancer, uterus cancer, espophagus cancer, liver cancer, an acoustic neuroma, oligodendroglioma, meningioma, neuroblastoma, or retinoblastoma. In certain other embodiments, the cancer is small cell lung cancer, non-small cell lung cancer, melanoma, cancer of the central nervous system tissue, brain cancer, Hodgkin's lymphoma, non-Hodgkin's lymphoma, cutaneous T-Cell lymphoma, cutaneous B-Cell lymphoma, or diffuse large B-Cell lymphoma. In certain other embodiments, the cancer is breast cancer, colon cancer, small-cell lung cancer, non-small cell lung cancer, prostate cancer, renal cancer, ovarian cancer, leukemia, melanoma, or cancer of the central nervous system tissue. In certain other embodiments, the cancer is colon cancer, small-cell lung cancer, non-small cell lung cancer, renal cancer, ovarian cancer, renal cancer, or melanoma.

In certain embodiments, the cancer is a fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, epithelial carcinoma, glioma, astrocytoma, medulloblastoma, or hemangioblastoma.

In certain embodiments, the cancer is a neuroblastoma, meningioma, hemangiopericytoma, multiple brain metastase, glioblastoma multiforms, glioblastoma, brain stem glioma, poor prognosis malignant brain tumor, malignant glioma, anaplastic astrocytoma, anaplastic oligodendroglioma, neuroendocrine tumor, rectal adeno carcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma, localized melanoma, malignant mesothelioma, malignant pleural effusion mesothelioma syndrome, peritoneal carcinoma, papillary serous carcinoma, gynecologic sarcoma, soft tissue sarcoma, scelroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waidenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, castrate resistant prostate cancer, castrate resistant metastatic prostate cancer, papillary thyroid carcinoma, follicular thyroid carcinoma, medullary thyroid carcinoma, or leiomyoma.

In certain embodiments, the cancer is bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In certain embodiments, the cancer is hepatocellular carcinoma, ovarian cancer, ovarian epithelial cancer, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In certain embodiments, the cancer is hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In certain embodiments, the cancer is a solid tumor, such as a sarcoma, carcinoma, or lymphoma. In certain embodiments, the cancer is kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In certain embodiments, the cancer is renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In certain embodiments, the cancer is hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In certain embodiments, the cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

### Causing Death of Cancer Cell

Another aspect of the invention provides a method of causing death of a cancer cell. The method comprises contacting a cancer cell with an effective amount of a compound described herein, such as a compound of Formula I or II, or other compound in Section I, to cause death of the cancer cell. In certain embodiments, the particular compound of Formula I or II is a compound defined by one of the embodiments described above.

In certain embodiments, the cancer cell is selected from ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct and gallbladder cancers, liver cancer, pancreatic cancer, bladder cancer, urinary tract cancer, kidney cancer, eye cancer, thyroid cancer, lymphoma, or leukemia. In certain embodiments, the cancer cell is one or more of the cancers recited in the section above entitled “Cancer.” In certain embodiments, the cancer cell is a prostate cancer cell.

### Combination Therapies

The compounds useful within the methods of the invention may be used in combination with one or more additional therapeutic agents useful for treating any disease contemplated herein. These additional therapeutic agents may comprise compounds that are commercially available or synthetically accessible to those skilled in the art. These additional therapeutic agents are known to treat, prevent, or reduce the symptoms, of a disease or disorder contemplated herein.

Accordingly, in certain embodiments, the method further comprises administering to the subject an additional therapeutic agent that treats the disease contemplated herein.

In certain embodiments, administering the compound of the invention to the subject allows for administering a lower dose of the additional therapeutic agent as compared to the dose of the additional therapeutic agent alone that is required to achieve similar results in treating the disease contemplated herein. For example, in certain embodiments, the compound of the invention enhances the therapeutic activity of the additional therapeutic compound, thereby allowing for a lower dose of the additional therapeutic compound to provide the same effect.

A synergistic effect may be calculated, for example, using suitable methods such as, for example, the Sigmoid-Ehd max equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

In certain embodiments, the compound of the invention and the therapeutic agent are co-administered to the subject. In other embodiments, the compound of the invention and the therapeutic agent are coformulated and co-administered to the subject.

In certain embodiments, the compound is administered in combination with a second therapeutic agent having activity against cancer. In certain embodiments, the second therapeutic agent is mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, and leutinizing hormone releasing factor.

In certain embodiments, the second therapeutic agent is an mTOR inhibitor, which inhibits cell proliferation, angiogenesis and glucose uptake. Approved mTOR inhibitors useful in the present invention include everolimus (Afinitor®, Novartis); temsirolimus (Torisel®, Pfizer); and sirolimus (Rapamune®, Pfizer).

In certain embodiments, the second therapeutic agent is a Poly ADP ribose polymerase (PARP) inhibitor. Approved PARP inhibitors useful in the present invention include olaparib (Lynparza®, AstraZeneca); rucaparib (Rubraca®, Clovis Oncology); and niraparib (Zejula®, Tesaro). Other PARP inhibitors being studied which may be used in the present invention include talazoparib (MDV3800/BMN 673/LT00673, Medivation/Pfizer/Biomarin); veliparib (ABT-888, AbbVie); and BGB-290 (BeiGene, Inc.).

In certain embodiments, the second therapeutic agent is a phosphatidylinositol 3 kinase (PI3K) inhibitor. Approved PI3K inhibitors useful in the present invention include idelalisib (Zydelig®, Gilead). Other PI3K inhibitors being studied which may be used in the present invention include alpelisib (BYL719, Novartis); taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

In certain embodiments, the second therapeutic agent is a proteasome inhibitor. Approved proteasome inhibitors useful in the present invention include bortezomib (Velcade®, Takeda); carfilzomib (Kyprolis®, Amgen); and ixazomib (Ninlaro®, Takeda).

In certain embodiments, the second therapeutic agent is a histone deacetylase (HDAC) inhibitor. Approved HDAC inhibitors useful in the present invention include vorinostat (Zolinza®, Merck); romidepsin (Istodax®, Celgene); panobinostat (Farydak®, Novartis); and belinostat (Beleodaq®, Spectrum Pharmaceuticals). Other HDAC inhibitors being studied which may be used in the present invention include entinostat (SNDX-275, Syndax Pharmaceuticals) (NCT00866333); and chidamide (Epidaza®, HBI-8000, Chipscreen Biosciences, China).

In certain embodiments, the second therapeutic agent is a CDK inhibitor, such as a CDK 4/6 inhibitor. Approved CDK 4/6 inhibitors useful in the present invention include palbociclib (Ibrance®, Pfizer); and ribociclib (Kisqali®, Novartis). Other CDK 4/6 inhibitors being studied which may be used in the present invention include abemaciclib (Ly2835219, Eli Lilly); and trilaciclib (G1T28, G1 Therapeutics).

In certain embodiments, the second therapeutic agent is an indoleamine (2,3)-dioxygenase (IDO) inhibitor. IDO inhibitors being studied which may be used in the present invention include epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS:F001287 (Bristol-Myers Squibb); Phy906/KD108 (Phytoceutica); and an enzyme that breaks down kynurenine (Kynase, Kyn Therapeutics).

In certain embodiments, the second therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (Lartruvo®; Eli Lilly). Approved EGFR antagonists which may be used in the present invention include cetuximab (Erbitux®, Eli Lilly); necitumumab (Portrazza®, Eli Lilly), panitumumab (Vectibix®, Amgen); and osimertinib (targeting activated EGFR, Tagrisso®, AstraZeneca).

In certain embodiments, the second therapeutic agent is an aromatase inhibitor. Approved aromatase inhibitors which may be used in the present invention include exemestane (Aromasin®, Pfizer); anastazole (Arimidex®, AstraZeneca) and letrozole (Femara®, Novartis).

In certain embodiments, the second therapeutic agent is an antagonist of the hedgehog pathway. Approved hedgehog pathway inhibitors which may be used in the present invention include sonidegib (Odomzo®, Sun Pharmaceuticals); and vismodegib (Erivedge®, Genentech), both for treatment of basal cell carcinoma.

In certain embodiments, the second therapeutic agent is a folic acid inhibitor. Approved folic acid inhibitors useful in the present invention include pemetrexed (Alimta®, Eli Lilly).

In certain embodiments, the second therapeutic agent is a CC chemokine receptor 4 (CCR4) inhibitor. CCR4 inhibitors being studied that may be useful in the present invention include mogamulizumab (Poteligeo®, Kyowa Hakko Kirin, Japan).

In certain embodiments, the second therapeutic agent is an isocitrate dehydrogenase (IDH) inhibitor. IDH inhibitors being studied which may be used in the present invention include AG120 (Celgene; NCT02677922); AG221 (Celgene, NCT02677922; NCT02577406); BAYl436032 (Bayer, NCT02746081); IDH305 (Novartis, NCT02987010).

In certain embodiments, the second therapeutic agent is an arginase inhibitor. Arginase inhibitors being studied which may be used in the present invention include AEB 1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In certain embodiments, the second therapeutic agent is a glutaminase inhibitor. Glutaminase inhibitors being studied which may be used in the present invention include CB-839 (Calithera Biosciences).

In certain embodiments, the second therapeutic agent is an antibody that binds to tumor antigens, that is, proteins expressed on the cell surface of tumor cells. Approved antibodies that bind to tumor antigens which may be used in the present invention include rituximab (Rituxan®, Genentech/BiogenIdec); ofatumumab (anti-CD20, Arzerra®, GlaxoSmithKline); obinutuzumab (anti-CD20, Gazyva®, Genentech), ibritumomab (anti-CD20 and Yttrium-90, Zevalin®, Spectrum Pharmaceuticals); daratumumab (anti-CD38, Darzalex®, Janssen Biotech), dinutuximab (anti-glycolipid GD2, Unituxin®, United Therapeutics); trastuzumab (anti-HER2, Herceptin®, Genentech); ado-trastuzumab emtansine (anti-HER2, fused to emtansine, Kadcyla®, Genentech); and pertuzumab (anti-HER2, Perjeta®, Genentech); and brentuximab vedotin (anti-CD30-drug conjugate, Adcetris®, Seattle Genetics).

In certain embodiments, the second therapeutic agent is a topoisomerase inhibitor. Approved topoisomerase inhibitors useful in the present invention include irinotecan (Onivyde®, Merrimack Pharmaceuticals); topotecan (Hycamtin®, GlaxoSmithKline). Topoisomerase inhibitors being studied which may be used in the present invention include pixantrone (Pixuvri®, CTI Biopharma).

In certain embodiments, the second therapeutic agent is a nucleoside inhibitor, or other therapeutic that interfere with normal DNA synthesis, protein synthesis, cell replication, or will otherwise inhibit rapidly proliferating cells. Such nucleoside inhibitors or other therapeutics include trabectedin (guanidine alkylating agent, Yondelis®, Janssen Oncology), mechlorethamine (alkylating agent, Valchlor®, Aktelion Pharmaceuticals); vincristine (Oncovin®, Eli Lilly; Vincasar®, Teva Pharmaceuticals; Marqibo®, Talon Therapeutics); temozolomide (prodrug to alkylating agent 5-(3-methyltriazen-1-yl)-imidazole-4-carboxamide (MTIC) Temodar®, Merck); cytarabine injection (ara-C, antimetabolic cytidine analog, Pfizer); lomustine (alkylating agent, CeeNU®, Bristol-Myers Squibb; Gleostine®, NextSource Biotechnology); azacitidine (pyrimidine nucleoside analog of cytidine, Vidaza®, Celgene); omacetaxine mepesuccinate (cephalotaxine ester) (protein synthesis inhibitor, Synribo®; Teva Pharmaceuticals); asparaginase Erwinia chrysanthemi (enzyme for depletion of asparagine, Elspar®, Lundbeck; Erwinaze®, EUSA Pharma); eribulin mesylate (microtubule inhibitor, tubulin-based antimitotic, Halaven®, Eisai); cabazitaxel (microtubule inhibitor, tubulin-based antimitotic, Jevtana®, Sanofi-Aventis); capacetrine (thymidylate synthase inhibitor, Xeloda®, Genentech); bendamustine (bifunctional mechlorethamine derivative, believed to form interstrand DNA cross-links, Treanda®, Cephalon/Teva); ixabepilone (semi-synthetic analog of epothilone B, microtubule inhibitor, tubulin-based antimitotic, Ixempra®, Bristol-Myers Squibb); nelarabine (prodrug of deoxyguanosine analog, nucleoside metabolic inhibitor, Arranon®, Novartis); clorafabine (prodrug of ribonucleotide reductase inhibitor, competitive inhibitor of deoxycytidine, Clolar®, Sanofi-Aventis); and trifluridine and tipiracil (thymidine-based nucleoside analog and thymidine phosphorylase inhibitor, Lonsurf®, Taiho Oncology).

In certain embodiments, the second therapeutic agent is a platinum-based therapeutic, also referred to as platins. Platins cause cross-linking of DNA, such that they inhibit DNA repair and/or DNA synthesis, mostly in rapidly reproducing cells, such as cancer cells. Approved platinum-based therapeutics which may be used in the present invention include cisplatin (Platinol®, Bristol-Myers Squibb); carboplatin (Paraplatin®, Bristol-Myers Squibb; also, Teva; Pfizer); oxaliplatin (Eloxitin® Sanofi-Aventis); and nedaplatin (Aqupla®, Shionogi). Other platinum-based therapeutics which have undergone clinical testing and may be used in the present invention include picoplatin (Poniard Pharmaceuticals); and satraplatin (JM-216, Agennix).

In certain embodiments, the second therapeutic agent is a taxane compound, which causes disruption of microtubules, which are essential for cell division. Approved taxane compounds which may be used in the present invention include paclitaxel (Taxol®, Bristol-Myers Squibb), docetaxel (Taxotere®, Sanofi-Aventis; Docefrez®, Sun Pharmaceutical), albumin-bound paclitaxel (Abraxane®; Abraxis/Celgene), and cabazitaxel (Jevtana®, Sanofi-Aventis). Other taxane compounds which have undergone clinical testing and may be used in the present invention include SID530 (SK Chemicals, Co.) (NCT00931008).

In certain embodiments, the second therapeutic agent is an inhibitor of anti-apoptotic proteins, such as BCL-2. Approved anti-apoptotics which may be used in the present invention include venetoclax (Venclexta®, AbbVie/Genentech); and blinatumomab (Blincyto®, Amgen). Other therapeutic agents targeting apoptotic proteins which have undergone clinical testing and may be used in the present invention include navitoclax (ABT-263, Abbott), a BCL-2 inhibitor (NCT02079740).

In certain embodiments, the second therapeutic agent is a selective estrogen receptor modulator (SERM), which interferes with the synthesis or activity of estrogens. Approved SERMs useful in the present invention include raloxifene (Evista®, Eli Lilly).

In certain embodiments, the second therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL) (NCT02909972; NCT02264613).

In certain embodiments, the second therapeutic agent is an inhibitor of transforming growth factor-beta (TGF-beta or TGF). Inhibitors of TGF-beta proteins being studied which may be used in the present invention include NIS793 (Novartis), an anti-TGF-beta antibody being tested in the clinic for treatment of various cancers, including breast, lung, hepatocellular, colorectal, pancreatic, prostate and renal cancer (NCT 02947165). In some embodiments, the inhibitor of TGF-beta proteins is fresolimumab (GC1008; Sanofi-Genzyme), which is being studied for melanoma (NCT00923169); renal cell carcinoma (NCT00356460); and non-small cell lung cancer (NCT02581787). Additionally, in some embodiments, the additional therapeutic agent is a TGF-beta trap, such as described in Connolly et al. (2012) Int'l J. Biological Sciences 8:964-978. One therapeutic compound currently in clinical trials for treatment of solid tumors is M7824 (Merck KgaA formerly MSB0011459X), which is a bispecific, anti-PD-L1/TGFβ trap compound (NCT02699515); and (NCTO2517398). M7824 is comprised of a fully human IgG1 antibody against PD-L1 fused to the extracellular domain of human TGF-beta receptor II, which functions as a TGFβ “trap.”

In certain embodiments, the second therapeutic agent is a cancer vaccine. In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (μmlygic®, BioVex/Amgen, previously known as T-VEC), a genetically modified oncolytic viral therapy approved for treatment of unresectable cutaneous, subcutaneous and nodal lesions in melanoma. In some embodiments, the additional therapeutic agent is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In certain embodiments, the second therapeutic agent is an immune checkpoint inhibitor selected from a PD-1 antagonist, a PD-L1 antagonist, or a CTLA-4 antagonist. In some embodiments, a compound disclosed herein or a pharmaceutically acceptable salt thereof is administered in combination with nivolumab (anti-PD-1 antibody, Opdivo®, Bristol-Myers Squibb); pembrolizumab (anti-PD-1 antibody, Keytruda®, Merck); ipilimumab (anti-CTLA-4 antibody, Yervoy®, Bristol-Myers Squibb); durvalumab (anti-PD-L1 antibody, μmfinzi®, AstraZeneca); or atezolizumab (anti-PD-L1 antibody, Tecentriq®, Genentech). Other immune checkpoint inhibitors suitable for use in the present invention include REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (Bavencio®, Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; and PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

Another aspect of the invention provides for the use of a compound described herein (such as a compound of Formula I, Formula II, or other compounds in Section I) in the manufacture of a medicament. In certain embodiments, the medicament is for treating a disease described herein, such as cancer.

Another aspect of the invention provides for the use of a compound described herein (such as a compound of Formula I, Formula II, or other compounds in Section I) for treating a medical disease, such a disease described herein (e.g., cancer).

### Evaluation of Cellular Growth Inhibition of HEK293 Cells and HeLa Cells

Compounds can be evaluated for ability to inhibit the proliferation of HEK293 cells or HeLa cells according to the following procedure. HEK293 and HeLa cells are cultured in DMEM medium supplemented with 10% fetal bovine serum and 1% Penn/Strep. Cells are seeded in white 384-well plates at 500 cells/well in 25 μL complete medium. Following seeding, plates are spun at 300×g for three minutes and cultured at 37° C. with 5% CO₂ in a humidified tissue culture incubator. After 24 hours, compounds are titrated in 100% DMSO and diluted in complete cell culture medium. A 25 μL aliquot of compound/media mixture is added to cells to bring total volume in well to 50 μL. DMSO alone is used as a negative control. Plates are then spun at 300×g for three minutes and stored at 37° C. with 5% CO₂ for three days. On Day 0 and Day 3 of compound treatment, cell viability is quantified with CellTiter-Glo 2.0 reagent (Promega). After equilibrating microplates at room temperature for 30 minutes, 25 μL CellTiter-Glo 2.0 reagent is dispensed into each well to bring total volume to 75 μL. Plates are mixed on shaker for 2 minutes at 500 rpm, followed by a 10-minute incubation at room temperature. Following a quick spin, luminescence readings are measured with an EnVision Plate Reader. Data is normalized to DMSO treated Day 0 and Day 3 readings. A four-parameter non-linear regression curve fit is applied to dose-response data in GraphPad Prism data analysis software to determine the half maximal growth inhibitory concentration (GI₅₀) for each compound.

### III. Pharmaceutical Compositions and Dosing Considerations

As indicated above, the invention provides pharmaceutical compositions, which comprise a therapeutically-effective amount of one or more of the compounds described above, formulated together with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdennally; or (8) nasally. In certain embodiments, the invention provides a pharmaceutical composition comprising a compound described herein (e.g., a compound of Formula I) and a pharmaceutically acceptable carrier.

The phrase “therapeutically effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising a heterobifunctional substituted phenylpyrimidinone or related compound described herein in a therapeutically effective amount for the treatment of a medical disorder described herein.

### IV. MEDICAL KITS

Another aspect of this invention is a kit comprising (i) a compound described herein, such as a compound of Formula I, and (ii) instructions for use, such as treating cancer.

### EXAMPLES

The invention now being generally described will be more readily understood by reference to the following examples, which are included merely for purposes of illustration of certain aspects and embodiments of the present invention, and are not intended to limit the invention.

### General Methods

All reactions were carried out under an atmosphere of dry nitrogen or argon. Glassware was oven-dried prior to use. Unless otherwise indicated, common reagents or materials were obtained from commercial sources and used without further purification. N,N-Diisopropylethylamine (DIPEA) was obtained anhydrous by distillation over potassium hydroxide. Tetrahydrofuran (THF), dichloromethane (CH₂C₁₋₂), and dimethylformamide (DMF) were dried by a PureSolV™ solvent drying system. PTLC refers to preparatory thin layer chromatographic separation. Abbreviations: HFIP (hexafluoroisopropanol), HEPES (4-(2-hydroxyethyl)-1-piperazineethanesulfonic acid. Flash column chromatography was performed using silica gel 60 (230-400 mesh). Analytical thin layer chromatography (TLC) was carried out on Merck silica gel plates with QF-254 indicator and visualized by UV or KMnO₄.

¹H and ¹³C NMR spectra were recorded on an Agilent DD₂ 500 (500 MHz ¹H; 125 MHz 13C) or Agilent DD₂ 600 (600 MHz ¹H; 150 MHz ¹³C) or Agilent DD₂ 400 (400 MHz ¹H; 100 MHz ¹³C) spectrometer at room temperature. Chemical shifts were reported in ppm relative to the residual CDCl₃ (S 7.26 ppm ¹H; δ 77.0 ppm ¹³C), CD₃OD (δ 3.31 ppm ¹H; δ 49.00 ppm 13C), or d₆-DMSO (δ 2.50 ppm ¹H; δ 39.52 ppm ¹³C). NMR chemical shifts were expressed in ppm relative to internal solvent peaks, and coupling constants were measured in Hz. (bs=broad signal). In most cases, only peaks of the major rotamer are reported.

Mass spectra were obtained using Agilent 1100 series LC/MSD spectrometers. Analytical HPLC analyses were carried out on 250×4.6 mm C-18 column using gradient conditions (10-100% B, flow rate=0.0 mL/min, 20 mi), or as described in the LC-MS Method tables.

Unless indicated otherwise, preparative HPLC was carried out on 250×21.2 mm C-18 column using gradient conditions (10-100%, flow rate=10.0 mL/min, 20 mm). The eluents used were: solvent A (H₂O with 0.1% TFA) and solvent B (CH₃CN with (v/v TFA). Final products were typically purified via reversed-phase HPLC, PTLC, or flash column chromatography.

LC-MS Method 01

| Instrument | | Agilent 1100 LC & Agilent G1956A | | |
|---|---|---|---|---|
| Software | | Agilent Chemstation Rev. B. 04.03[54] | | |
| HPLC | Column | Agilent ZORBAX 5 μm SB-Aq, 2.1 × 50 mm | | |
| | Mobile Phase | A: 0.0375% TFA in water (v/v) | | |
| | | B: 0.01875% TFA in Acetonitrile (v/v) | | |
| | | Time | B | Flow |
| | | (min) | (%) | (mL/min) |
| | Gradient | 0.00 | 1 | 0.8 |
| | | 0.40 | 1 | 0.8 |
| | | 3.40 | 90 | 0.8 |
| | | 3.90 | 100 | 0.8 |
| | | 3.91 | 1 | 0.8 |
| | | 4.00 | 1 | 1.0 |
| | | 4.50 | 1 | 1.0 |

| | | |
|---|---|---|
| | Post time(min) | 0 |
| | Column Temp | 50° C. |
| | Detector | DAD |
| MS | Ionization | ESI |
| | source | |
| | Drying Gas | N₂ |
| | Drying Gas | 10 (L/min) |
| | Flow | |
| | Nebulizer | 40 (psi) |
| | Pressure | |
| | Drying Gas | 350° C. |
| | Temperature | |
| | Capillary | 2500 (V) |
| | Voltage | |
| | MS Polarity | Positive |
| | MS Mode | Scan |
| | Mass Range | 100-1500 |

LC-MS Method 05

| Instrument | | Agilent 1260 LC & Agilent 6125C MSD | | |
|---|---|---|---|---|
| Software | | Agilent Open Lab CDS 2.6 | | |
| HPLC | Column | Agilent poroshell 120 EC-C18 3.0 × 50 mm, | | |
| | | 2.7 μm | | |
| | Mobile Phase | A: 0.0375% TFA in water (v/v) | | |
| | | B: 0.01875% TFA in Acetonitrile (v/v) | | |
| | Gradient | Time (min) | B (%) | Flow (mL/min) |
| | | 0.00 | 05 | 1.0 |
| | | 0.40 | 05 | 1.0 |
| | | 3.00 | 99 | 1.0 |
| | | 4.00 | 99 | 1.0 |
| | | 4.10 | 5 | 1.0 |
| | | 4.50 | 5 | 1.0 |

| | | |
|---|---|---|
| | Post time(min) | Off |
| | Column Temp | 45° C. |
| | Detector | DAD |
| MS | Ionization source | ESI |
| | Drying Gas | N₂ |
| | Drying Gas Flow | 10 (L/min) |
| | Nebulizer | 40 (psi) |
| | Pressure | |
| | Drying Gas | 350° C. |
| | Temperature | |
| | Capillary Voltage | 3500(V) Positive |
| | MS Polarity | Positive |
| | MS Mode | Scan |
| | Mass Range | 100-2000 |

LC-MS Method 10

| Instrument | | Agilent 1100 LC & Agilent G1956A | | |
|---|---|---|---|---|
| Software | | Agilent Chemstation Rev. B. 04.03[54] | | |
| HPLC | Column | Agilent ZORBAX 5 μm SB-Aq, 2.1 × 50 | | |
| | | mm | | |
| | Mobile Phase | A: 0.0375% TFA in water (v/v) | | |
| | | B: 0.01875% TFA in Acetonitrile (v/v) | | |
| | Gradient | Time (min) | B (%) | Flow (mL/min) |
| | | 0.00 | 10 | 0.8 |
| | | 0.40 | 10 | 0.8 |
| | | 3.40 | 100 | 0.8 |
| | | 3.90 | 100 | 0.8 |
| | | 3.91 | 10 | 0.8 |
| | | 4.00 | 10 | 1.0 |
| | | 4.50 | 10 | 1.0 |

| | | |
|---|---|---|
| | Post time(min) | 0 |
| | Column Temp | 50° C. |
| | Detector | DAD |
| MS | Ionization source | ESI |
| | Drying Gas | N₂ |
| | Drying Gas Flow | 10 (L/min) |
| | Nebulizer | 40 (psi) |
| | Pressure | |
| | Drying Gas | 350° C. |
| | Temperature | |
| | Capillary Voltage | 2500 (V) |
| | MS Polarity | Positive |
| | MS Mode | Scan |
| | Mass Range | 100-1500 |

LC-MS Method 25

| Instrument | | Agilent 1100 LC & Agilent G1956A | | |
|---|---|---|---|---|
| Software | | Agilent Chemstation Rev. B. 04.03[54] | | |
| HPLC | Column | Agilent ZORBAX 5 μm SB-Aq, 2.1 × 50 | | |
| | | mm | | |
| | Mobile Phase | A: 0.0375% TFA in water (v/v) | | |
| | | B: 0.01875% TFA in Acetonitrile (v/v) | | |
| | Gradient | Time (min) | B (%) | Flow (mL/min) |
| | | 0.00 | 25 | 0.8 |
| | | 0.40 | 25 | 0.8 |
| | | 3.40 | 100 | 0.8 |
| | | 3.90 | 100 | 0.8 |
| | | 3.91 | 25 | 0.8 |
| | | 4.00 | 25 | 1.0 |
| | | 4.50 | 25 | 1.0 |

| | | |
|---|---|---|
| | Post time(min) | 0 |
| | Column Temp | 50° C. |
| | Detector | DAD |
| MS | Ionization source | ESI |
| | Drying Gas | N₂ |
| | Drying Gas Flow | 10 (L/min) |
| | Nebulizer | 40 (psi) |
| | Pressure | |
| | Drying Gas | 350° C. |
| | Temperature | |
| | Capillary | 2500(V) Positive |
| | Voltage | |
| | MS Polarity | Positive |
| | MS Mode | Scan |
| | Mass Range | 100-1500 |

LC-MS METHOD 40

| Instrument | | Agilent 1100 LC & Agilent G1956A | | |
|---|---|---|---|---|
| Software | | Agilent Chemstation Rev. B. 04.03[16] | | |
| HPLC | Column | Agilent ZORBAX 5 μm SB-Aq, 2.1 × 50 | | |
| | | mm | | |
| | Mobile Phase | A: 0.0375% TFA in water (v/v) | | |
| | | B: 0.01875% TFA in Acetonitrile (v/v) | | |
| | Gradient | Time (min) | B (%) | Flow (mL/min) |
| | | 0.00 | 40 | 0.8 |
| | | 0.40 | 40 | 0.8 |
| | | 3.40 | 100 | 0.8 |
| | | 3.90 | 100 | 0.8 |
| | | 3.91 | 40 | 0.8 |
| | | 4.00 | 40 | 1.0 |
| | | 4.50 | 40 | 1.0 |

| | | |
|---|---|---|
| | Post time(min) | 0 |
| | Column Temp | 50° C. |
| | Detector | DAD (Agilent 1100)/ELSD (Agilent 1260 |
| | | Infinity) |
| MS | Ionization source | ESI |
| | Drying Gas | N₂ |
| | Drying Gas Flow | 10 (L/min) |
| | Nebulizer | 2070 (Torr) |
| | Pressure | |
| | Drying Gas | 350° C. |
| | Temperature | |
| | Capillary Voltage | 2500 (V) Positive |

LC-MS Method 80

| Instrument | | Agilent 1290 LC & Agilent 6125C MSD | | |
|---|---|---|---|---|
| Software | | Agilent Open Lab CDS 2.6 | | |
| HPLC | Column | Agilent Poroshell EC C18, 2.1*50 mm, 1.9 | | |
| | | μm | | |
| | Mobile Phase | A: 0.0375% TFA in water (v/v) | | |
| | | B: 0.01875% TFA in Acetonitrile (v/v) | | |
| | Gradient | Time(min) | B(%) | Flow(mL/min) |
| | | 0.00 | 10 | 1.0 |
| | | 1.50 | 80 | 1.0 |
| | | 3.00 | 100 | 1.0 |

| | | |
|---|---|---|
| | Post time(min) | 1.0 |
| | Column Temp | 50° C. |
| | Detector | DAD |
| MS | Ionization source | ESI |
| | Drying Gas | N₂ |
| | Drying Gas Flow | 10(L/min) |
| | Nebulizer | 40(psi) |
| | Pressure | |
| | Drying Gas | 350° C. |
| | Temperature | |
| | Capillary | 4000(V) Positive |
| | Voltage | |
| | Fragmentor | 110 V |
| | MS Polarity | Positive |
| | MS Mode | Scan |
| | Mass Range | 100-2000 |

The following abbreviations are used herein: ACN: acetonitrile; Bn: benzyl; Boc: tert-butoxycarbonyl; DCM: dichloromethane; DIEA: diisopropylethylamine; DMF: dimethylformamide; DMSO: dimethylsulfoxide; EtOH: ethanol; EA or EtOAc:ethyl acetate; equiv. or eq.: molar equivalents; FA: formic acid; h: hour or hours; HATU: 1-[bis(dimethylamino)methylene]-1H-1,2,3-triazolo[4,5-b]pyridinium 3-oxide hexafluorophosphate; HPLC: higH-pressure liquid chromatography; LCMS or LC-MS: liquid chromatography-mass spectrometry; MeCN: acetonitrile; MeOH: methanol; MS: mass spectrometry; NMP: N-methylpyrrolidone; NMR: nuclear magnetic resonance, PE: petroleum ether; rt: room temperature; TEA: triethylamine; TFA: trifluoroacetic acid; THF: tetrahydrofuran; TLC: thin-layer chromatography; psi: pounds-per-square inch; and Tos or Ts: p-toluenesulfonyl.

### Example 1—Synthesis of N-((1r, 3r)-3-(3-chloro-4-cyanophenoxy)-2,2,4,4-tetramethylcyclobutyl)-2-((2-(3-(4-(ethylsulfonamido)-2-(6-methyl-7-oxo-6,7-dihydro-1H-pyrrolo[2,3-c]pyridin-4-yl)phenoxy)phenoxy)ethyl)(methyl)amino)pyrimidine-5-carboxamide (I-1)

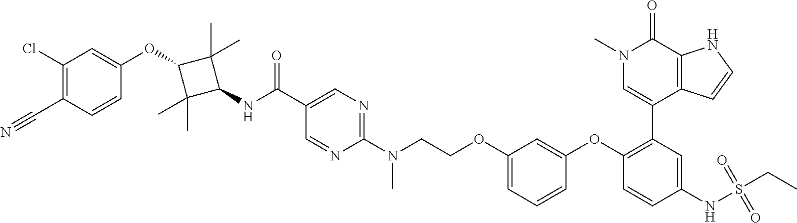 [SMILES: CCS(=O)(=O)Nc1ccc(Oc2cccc(OCCN(C)c3ncc(C(=O)N[C@H]4C(C)(C)[C@H](Oc5ccc(C#N)c(Cl)c5)C4(C)C)cn3)c2)c(-c2cn(C)c(=O)c3[nH]ccc23)c1]

### Step 1: Synthesis of 1-(benzyloxy)-3-(2-bromoethoxy)benzene

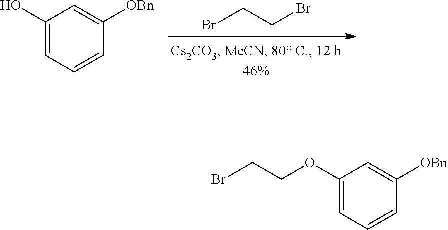

To a solution of 1,2-dibromoethane (30.0 g, 160 μmol, 12 mL, 6.4 equiv) in MeCN (75 mL) was added Cs₂CO₃ (17.5 g, 53.7 mol, 2.2 equiv) and 3-(benzyloxy)phenol (5.00 g, 25.0 μmol, 1.0 equiv). The mixture was stirred at 80° C. for 12 hours. The reaction mixture was filtered and concentrated under reduced pressure to give a residue. The residue was purified by column chromatography (SiO₂, petroleum ether/ethyl acetate=15/1 to 6/1). 1-(Benzyloxy)-3-(2-bromoethoxy)benzene (3.50 g, 11.4 μmol, 46% yield) was obtained as a yellow oil. ¹H NMR (400 MHz, CHLOROFORM-d): δ 3.57-3.84 (m, 2H) 4.17-4.45 (m, 2H) 5.12 (s, 2H) 6.51-6.76 (m, 3H) 7.23-7.54 (m, 1H) 7.27 (t, J=8.25 Hz, 1H) 7.33 (s, 1H) 7.38-7.38 (m, 1H) 7.38-7.44 (m, 1H) 7.44-7.54 (m, 1H).

### Step 2: Synthesis of N-benzyl-2-(3-(benzyloxy)phenoxy)-N-methylethan-1-amine

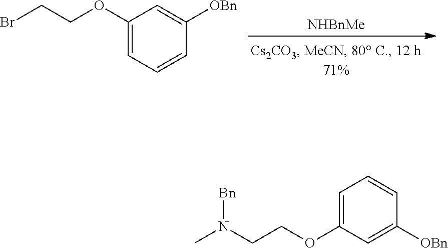 [SMILES: BrCCOc1cccc(OCc2ccccc2)c1 ; CN(CCOc1cccc(OCc2ccccc2)c1)Cc1ccccc1 ; "NHBnMe"]

To a solution of 1-(benzyloxy)-3-(2-bromoethoxy)benzene (3.50 g, 11.4 μmol, 1.0 equiv) in MeCN (72 mL) was added Cs₂CO₃ (8.75 g, 26.9 mol, 2.4 equiv) and N-methyl-1-phenyl-methanamine (1.75 g, 14.4 μmol, 1.9 mL, 1.3 equiv). The mixture was stirred at 80° C. for 12 hours. The reaction mixture was filtered and concentrated under reduced pressure to give a residue. The residue was purified by column chromatography (SiO₂, petroleum ether/ethyl acetate=10/1 to 4/1). N-Benzyl-2-(3-(benzyloxy)phenoxy)-N-methylethan-1-amine (2.80 g, 8.06 mol, 71% yield) was obtained as a yellow oil. ¹H NMR (400 MHz, CHLOROFORM-d): δ 2.28-2.30 (m, 3H) 2.75-2.81 (m, 2H) 3.57 (s, 2H) 3.99-4.06 (m, 2H) 4.96-5.02 (m, 2H) 6.44-6.57 (m, 3H) 7.09-7.15 (m, 1H) 7.17-7.23 (m, 1H) 7.26-7.40 (m, 8H).

### Step 3: Synthesis of tert-butyl (2-(3-hydroxyphenoxy)ethyl)(methyl) carbamate

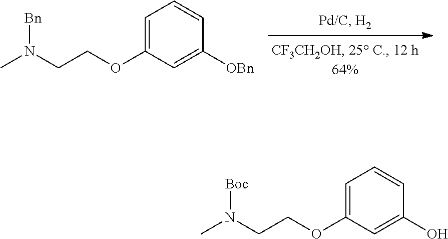 [SMILES: CN(CCOc1cccc(OCc2ccccc2)c1)Cc1ccccc1 ; "Pd/C, H2" ; CN(CCOc1cccc(O)c1)C(=O)OC(C)(C)C]

To a solution of N-benzyl-2-(3-(benzyloxy)phenoxy)-N-methylethan-1-amine (2.80 g, 8.06 mol, 1 equiv) and (Boc)₂0 (3.87 g, 17.7 μmol, 4.1 mL, 2.2 equiv) in CF₃CH₂OH (30 mL) was added Pd/C (2.80 g, 2.63 mol, 10% purity, 0.3 equiv) under N₂ atmosphere. The suspension was degassed and purged with H₂ three times. The mixture was stirred under H₂ (15 psi) at 25° C. for 12 hours. The reaction mixture was filtered and concentrated under reduced pressure to remove solvent. The residue was purified by column chromatography (SiO₂, petroleum ether/ethyl acetate=10/1 to 1/1) to give tert-butyl (2-(3-hydroxyphenoxy) ethyl)(methyl) carbamate (1.38 g, 5.16 mol, 64% yield) as a white solid. ¹H NMR (400 MHz, CHLOROFORM-d): δ 1.42-1.51 (m, 9H) 1.58-1.66 (m, 2H) 1.70-1.98 (m, 6H) 3.03-3.18 (m, 2H) 3.36-3.62 (m, 3H) 3.70-3.84 (m, 2H) 3.91-4.05 (m, 2H) 6.30-6.58 (m, 3H) 7.04-7.17 (m, 1H).

### Step 4: Synthesis of tert-butyl N-methyl-N-[2-[3-[2-(6-methyl-7-oxo-1H-pyrrolo[2,3-c]pyridin-4-yl)-4-nitro-phenoxy]phenoxy]ethyl]carbamate

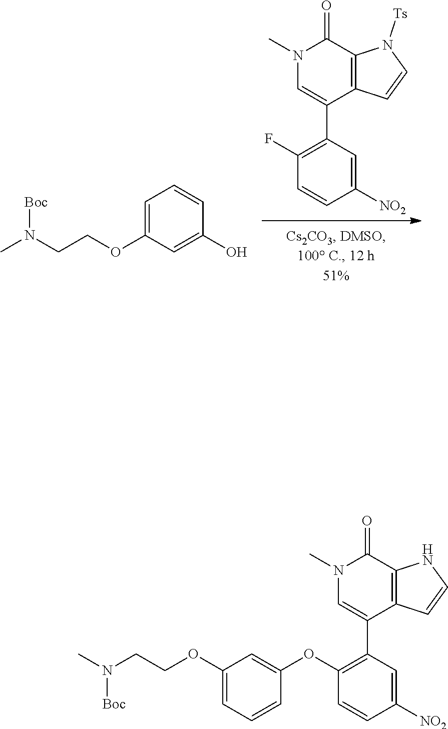 [SMILES: CN(CCOc1cccc(O)c1)C(=O)OC(C)(C)C ; Cc1ccc(S(=O)(=O)n2ccc3c(-c4cc([N+](=O)[O-])ccc4F)cn(C)c(=O)c32)cc1 ; CN(CCOc1cccc(Oc2ccc([N+](=O)[O-])cc2-c2cn(C)c(=O)c3[nH]ccc23)c1)C(=O)OC(C)(C)C]

To a solution of tert-butyl N-[2-(3-hydroxyphenoxy)ethyl]-N-methyl-carbamate (260 mg, 973 μmol, 1.2 equiv) in DMSO (8.0 mL) was added Cs₂CO₃ (650 mg, 1.99 μmol, 2.5 equiv) and 4-(2-fluoro-5-nitro-phenyl)-6-methyl-1-(p-tolylsulfonyl)pyrrolo[2,3-c]pyridin-7-one (350 mg, 793 μmol, 1.0 equiv) at 25° C. The mixture was stirred at 100° C. for 2 hours. LCMS showed the reaction was completed. The reaction mixture was partitioned between H₂O (15 mL) and EtOAc (200 mL). The organic phase was separated, washed with saturated salt solution (20 mL×5), dried over anhydrous sodium sulfate, filtered, and concentrated under reduced pressure to give a residue. The residue was purified by column chromatography (SiO₂, petroleum ether/ethyl acetate=1/1 to 5/1). Compound tert-butyl N-methyl-N-[2-[3-[2-(6-methyl-7-oxo-1H-pyrrolo[2,3-c]pyridin-4-yl)-4-nitro-phenoxy]phenoxy]ethyl]carbamate (216 mg, 400 μmol, 51% yield) was obtained as a yellow solid. ¹H NMR (400 MHz, CHLOROFORM-d): δ ppm 1.19-1.32 (m, 1H) 1.40-1.49 (m, 2H) 1.55-1.59 (m, 9H) 1.99-2.10 (m, 1H) 2.89-3.01 (m, 1H) 3.36 (s, 1H) 3.54-3.64 (m, 1H) 3.65-3.76 (m, 1H) 3.96 (s, 1H) 6.36-6.43 (m, 1H) 6.50-6.76 (m, 1H) 6.93-7.07 (m, 1H) 7.24-7.31 (m, 4H) 7.41-7.54 (m, 1H) 8.12-8.19 (m, 1H) 8.40-8.50 (m, 1H) 9.46 (s, 1H).

### Step 5: Synthesis of tert-butyl (2-(3-(4-amino-2-(6-methyl-7-oxo-6,7-dihydro-1H-pyrrolo[2,3-c]pyridin-4-yl)phenoxy)phenoxy)ethyl)(methyl)carbamate

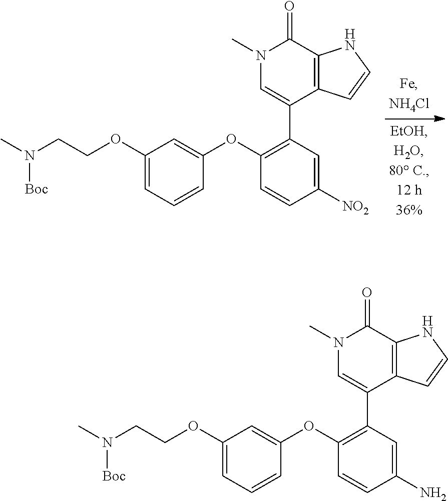 [SMILES: CN(CCOc1cccc(Oc2ccc([N+](=O)[O-])cc2-c2cn(C)c(=O)c3[nH]ccc23)c1)C(=O)OC(C)(C)C ; CN(CCOc1cccc(Oc2ccc(N)cc2-c2cn(C)c(=O)c3[nH]ccc23)c1)C(=O)OC(C)(C)C]

To a solution of tert-butyl N-methyl-N-[2-[3-[2-(6-methyl-7-oxo-1H-pyrrolo[2,3-c]pyridin-4-yl)-4-nitro-phenoxy]phenoxy]ethyl]carbamate (300 mg, 561 mol, 1.0 equiv) and NH₄Cl (150 mg, 2.81 μmol, 5.0 equiv) in EtOH (40 M1) and H₂O (20 M1) was added Fe (157 mg, 2.81 mol, 5.0 equiv) at 25° C. The mixture was stirred at 85° C. for 2 hours. LCMS showed the reaction was completed. The reaction mixture was quenched by the addition of saturated sodium bicarbonate (5 mL) at 25° C. The mixture was then diluted with EtOAc (50 mL) and extracted with EtOAc (10 mL×5). The combined organic layers were washed with saturated salt solution (10 mL×3), dried over Na₂SO₄, filtered, and concentrated under reduced pressure to give a residue. The residue was purified by prep-TLC (SiO₂, DCM/MeOH=10/1). Compound tert-butyl (2-(3-(4-amino-2-(6-methyl-7-oxo-6,7-dihydro-1H-pyrrolo[2,3-c]pyridin-4-yl)phenoxy)phenoxy)ethyl)(methyl)carbamate (103 mg, 204 μmol, 36% yield) was obtained as a white solid.

### Step 6: Synthesis of tert-butyl (2-(3-(4-(ethylsulfonamido)-2-(6-methyl-7-oxo-6,7-dihydro-1H-pyrrolo[2,3-c]pyridin-4-yl)phenoxy)phenoxy)ethyl)(methyl)carbamate

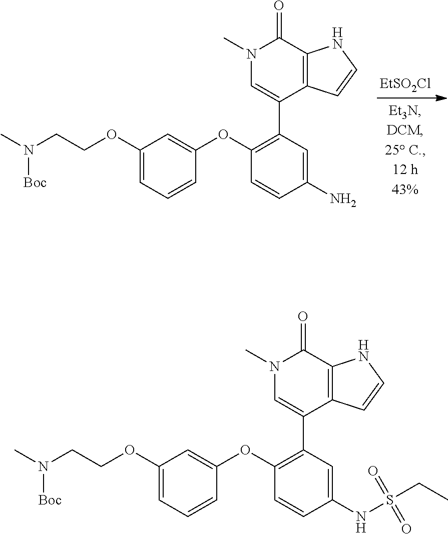 [SMILES: "EtSO2Cl" ; CN(CCOc1cccc(Oc2ccc(N)cc2-c2cn(C)c(=O)c3[nH]ccc23)c1)C(=O)OC(C)(C)C ; CCS(=O)(=O)Nc1ccc(Oc2cccc(OCCN(C)C(=O)OC(C)(C)C)c2)c(-c2cn(C)c(=O)c3[nH]ccc23)c1]

To a solution of tert-butyl (2-(3-(4-amino-2-(6-methyl-7-oxo-6,7-dihydro-1H-pyrrolo[2,3-c]pyridin-4-yl)phenoxy)phenoxy)ethyl)(methyl)carbamate (90 mg, 178 μmol, 1.0 equiv) in DCM (5.0 Ml) was added Et₃N (36 mg, 0.36 mol, 50 Ml, 2.0 equiv) and ethanesulfonyl chloride (23 mg, 0.18 μmol, 17 Ml, 1.0 equiv) at 0° C. The mixture was stirred at 0-25° C. for 2 hours. LCMS showed the reaction was completed. The reaction mixture was quenched by addition of CH₃OH (2 mL) at 0° C., and then concentrated under reduced pressure to give a residue. The residue was purified by prep-HPLC (column: Phenomenex luna C18 150 mm×25 mm, 10 μm; mobile phase: [water(FA)-can]; B %: 42%-72%, 10 min). Compound tert-butyl (2-(3-(4-(ethylsulfonamido)-2-(6-methyl-7-oxo-6,7-dihydro-1H-pyrrolo[2,3-c]pyridin-4-yl)phenoxy)phenoxy)ethyl)(methyl)carbamate (46 mg, 77 mol, 43% yield) was obtained as a white oil.

### Step 7: Synthesis of N-(3-(6-methyl-7-oxo-6,7-dihydro-1H-pyrrolo[2,3-c]pyridin-4-yl)-4-(3-(2-(methylamino)ethoxy)phenoxy)phenyl)ethanesulfonamide

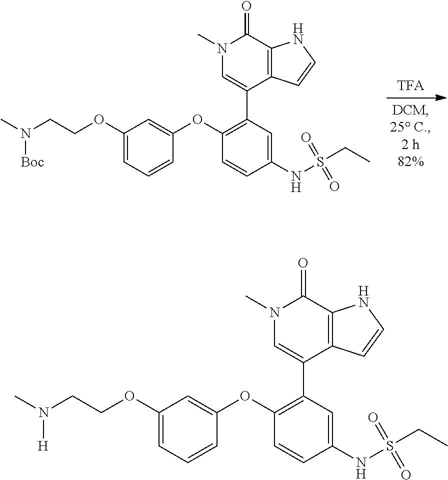

To a solution of tert-butyl (2-(3-(4-(ethylsulfonamido)-2-(6-methyl-7-oxo-6,7-dihydro-1H-pyrrolo[2,3-c]pyridin-4-yl)phenoxy)phenoxy)ethyl)(methyl)carbamate (35.0 mg, 58.7 μmol, 1.0 equiv) in DCM (4.0 mL) was added TFA (6.69 mg, 58.7 μmol, 4.34 μL, 1.0 equiv). The mixture was stirred at 25° C. for 1 hour. LCMS showed the reaction was completed. The reaction mixture was concentrated under reduced pressure to give a residue. The crude product, N-(3-(6-methyl-7-oxo-6,7-dihydro-1H-pyrrolo[2,3-c]pyridin-4-yl)-4-(3-(2-(methylamino)ethoxy)phenoxy)phenyl)ethanesulfonamide (24.0 mg, 48.3 mol, 82% yield), was obtained as a yellow oil and used in the next step without further purification.

### Step 8: Synthesis of N-((1r, 3r)-3-(3-chloro-4-cyanophenoxy)-2,2,4,4-tetramethylcyclobutyl)-2-((2-(3-(4-(ethylsulfonamido)-2-(6-methyl-7-oxo-6,7-dihydro-1H-pyrrolo[2,3-c]pyridin-4-yl)phenoxy)phenoxy)ethyl)(methyl)amino)pyrimidine-5-carboxamide (I-1)

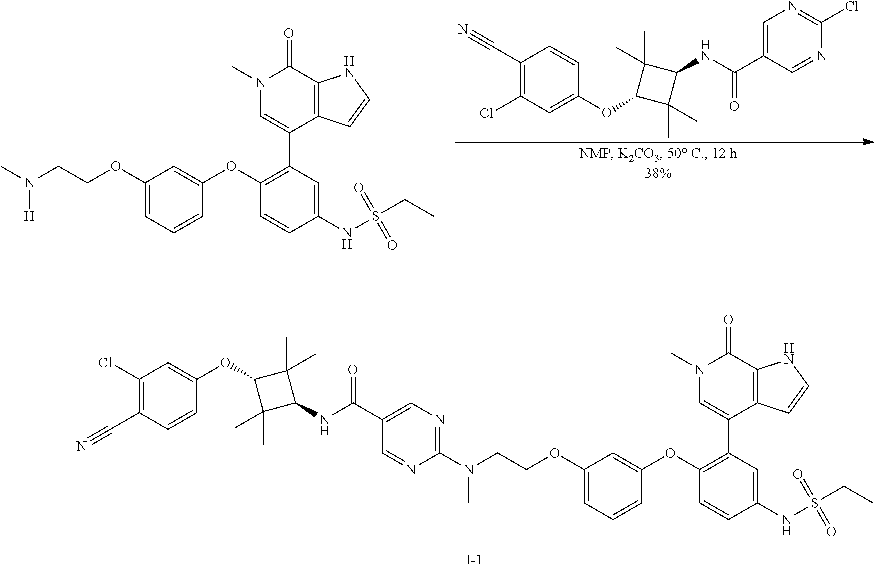 [SMILES: CCS(=O)(=O)Nc1ccc(Oc2cccc(OCCNC)c2)c(-c2cn(C)c(=O)c3[nH]ccc23)c1 ; CC1(C)[C@H](NC(=O)c2cnc(Cl)nc2)C(C)(C)[C@H]1Oc1ccc(C#N)c(Cl)c1 ; CCS(=O)(=O)Nc1ccc(Oc2cccc(OCCN(C)c3ncc(C(=O)N[C@H]4C(C)(C)[C@H](Oc5ccc(C#N)c(Cl)c5)C4(C)C)cn3)c2)c(-c2cn(C)c(=O)c3[nH]ccc23)c1 ; [I-]]

To a solution of N-(3-(6-methyl-7-oxo-6,7-dihydro-1H-pyrrolo[2,3-c]pyridin-4-yl)-4-(3-(2-(methylamino)ethoxy)phenoxy)phenyl)ethanesulfonamide (24 mg, 48 μmol, 1.0 equiv) in NMP (4.0 mL) was added K₂CO₃ (13 mg, 97 mol, 2.0 equiv). Next, 2-chloro-N-[3-(3-chloro-4-cyano-phenoxy)-2,2,4,4-tetramethyl-cyclobutyl]pyrimidine-5-carboxamide (20 mg, 48 mol, 1.0 equiv) was added. The mixture was stirred at 50° C. for 12 hours. LCMS showed the reaction was completed. The reaction mixture was concentrated under reduced pressure to give a residue. The residue was purified by prep-HPLC (column: Waters Xbridge 150 mm×25 mm, 5 μm; mobile phase: [water(NH₄HCO₃)-ACN]; B %: 54%-84%, 10 min). Compound N-((1r, 3r)-3-(3-chloro-4-cyanophenoxy)-2,2,4,4-tetramethylcyclobutyl)-2-((2-(3-(4-(ethylsulfonamido)-2-(6-methyl-7-oxo-6,7-dihydro-1H-pyrrolo[2,3-c]pyridin-4-yl)phenoxy)phenoxy)ethyl)(methyl) amino)pyrimidine-5-carboxamide (16.0 mg, 18.2 mol, 38% yield) was obtained as a white solid. ¹H NMR (400 MHz, METHANOL-d₄): δ 1.19-1.23 (m, 6H) 1.27 (s, 6H) 1.32-1.39 (m, 3H) 3.08-3.19 (m, 2H) 3.26 (s, 3H) 3.42-3.51 (m, 1H) 3.95-4.03 (m, 2H) 4.05-4.10 (m, 2H) 4.12-4.18 (m, 1H) 4.24-4.30 (m, 1H) 6.26-6.39 (m, 3H) 6.46-6.54 (m, 1H) 6.93-7.14 (m, 4H) 7.20 (br d, J=2.13 Hz, 1H) 7.26-7.35 (m, 2H) 7.43-7.51 (m, 1H) 7.72 (d, J=8.76 Hz, 1H) 8.70-8.76 (m, 2H).

### Example 2—Synthesis of N-((1r, 3r)-3-(3-chloro-4-cyanophenoxy)-2,2,4,4-tetramethylcyclobutyl)-2-(4-((3-(4-(ethylsulfonamido)-2-(6-methyl-7-oxo-6,7-dihydro-1H-pyrrolo[2,3-c]pyridin-4-yl)phenoxy)benzyl)oxy)piperidin-1-yl)pyrimidine-5-carboxamide (I-18)

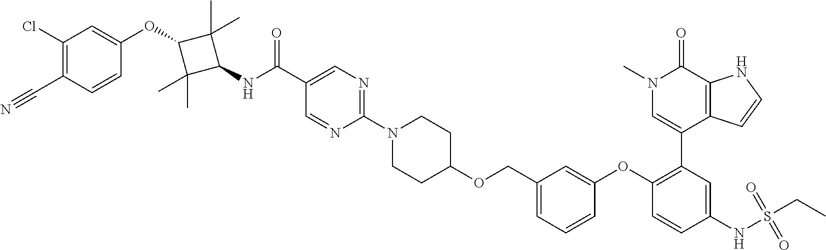 [SMILES: CCS(=O)(=O)Nc1ccc(Oc2cccc(COC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1]

### Step 1: Synthesis of tert-butyl 4-[(3-benzyloxyphenyl)methoxy]piperidine-1-carboxylate

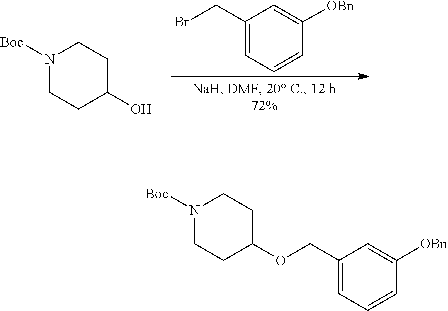 [SMILES: CC(C)(C)OC(=O)N1CCC(O)CC1 ; BrCc1cccc(OCc2ccccc2)c1 ; CC(C)(C)OC(=O)N1CCC(OCc2cccc(OCc3ccccc3)c2)CC1]

To a solution of tert-butyl 4-hydroxypiperidine-1-carboxylate (5 g, 25 mol, 1 equiv) in THF (40 mL) was added NaH (1.19 g, 30 mol, 60% purity, 1.2 equiv) at 0° C. for 0.5 hour. Next, 1-benzyloxy-3-(bromomethyl)benzene (6.9 g, 25 μmol, 1 equiv) was added at 0° C. The mixture was stirred at 20° C. for 12 hours and concentrated. The residue was purified by column chromatography (SiO₂, petroleum ether/ethyl acetate=50/1 to 10/1) to give tert-butyl 4-[(3-benzyloxyphenyl)methoxy]piperidine-1-carboxylate (7.15 g, 72% yield) as a yellow oil. ¹H NVR (400 MHz, CDC₃): δ 1.49 (s, 9H), 1.59˜1.61 (m, 2H), 1.85-1.89 (m, 2H), 3.11˜3.13 (m, 2H), 3.56˜3.59 (m, 1H), 3.73-3.84 (m, 2H), 4.55 (s, 2H), 5.10 (s, 2H), 6.88-6.97 (m, 2H), 7.01 (s, 1H), 7.26-7.30 (m, 1H), 7.32-7.43 (m, 3H), 7.43-7.49 (m, 2H).

### Step 2: Synthesis of tert-butyl 4-[(3-hydroxyphenyl)methoxy]piperidine-1-carboxylate

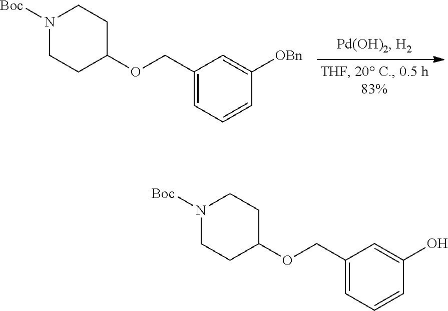 [SMILES: CC(C)(C)OC(=O)N1CCC(OCc2cccc(OCc3ccccc3)c2)CC1 ; CC(C)(C)OC(=O)N1CCC(OCc2cccc(O)c2)CC1]

To a solution of tert-butyl 4-[(3-benzyloxyphenyl)methoxy]piperidine-1-carboxylate (1 g, 2.52 μmol, 1 equiv) in THF (10 mL) was added Pd(OH)₂ (353 mg, 2.52 mol, 1 equiv) under N₂ atmosphere. The mixture was stirred under H₂ (15 psi) at 20° C. for 0.5 hour. The residue was purified by column chromatography (SiO₂, petroleum ether/ethyl acetate=20/1 to 10/1) to give tert-butyl 4-[(3-hydroxyphenyl)methoxy]piperidine-1-carboxylate (643 mg, 83% yield) as a white solid. ¹H NMR (400 MHz, CDCl₃): δ 1.48 (s, 9H), 1.61-1.66 (m, 1H), 1.88˜1.91 (m, 2H), 3.13˜3.15 (m, 2H), 3.58-3.61 (m, 1H), 3.75-3.86 (m, 2H), 4.53 (s, 2H), 5.20 (s, 1H), 6.77˜6.79 (m, 1H), 6.82-6.95 (m, 2H), 7.22˜7.24 (m, 1H).

### Step 3: Synthesis of tert-butyl 4-[[3-[2-(6-methyl-7-oxo-1H-pyrrolo[2,3-c]pyridin-4-yl)-4-nitro-phenoxy]phenyl]methoxy]piperidine-1-carboxylate

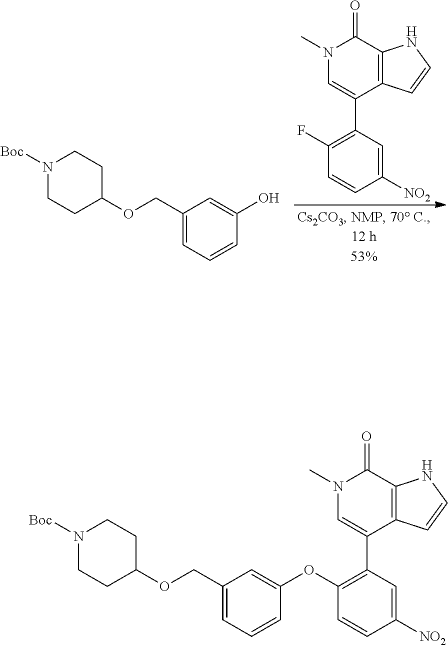 [SMILES: CC(C)(C)OC(=O)N1CCC(OCc2cccc(O)c2)CC1 ; Cn1cc(-c2cc([N+](=O)[O-])ccc2F)c2cc[nH]c2c1=O ; Cn1cc(-c2cc([N+](=O)[O-])ccc2Oc2cccc(COC3CCN(C(=O)OC(C)(C)C)CC3)c2)c2cc[nH]c2c1=O]

To a solution of tert-butyl 4-[(3-hydroxyphenyl)methoxy]piperidine-1-carboxylate (188 mg, 612 μmol, 1 equiv) and 4-(2-fluoro-5-nitro-phenyl)-6-methyl-1-(p-tolylsulfonyl) pyrrolo[2,3-c]pyridin-7-one (270 mg, 612 mol, 1 equiv) in NMP (3 mL) was added Cs₂CO₃ (598 mg, 1.83 μmol, 3 equiv). The mixture was stirred at 70° C. for 12 hours and concentrated. The residue was purified by column chromatography (SiO₂, petroleum ether/ethyl acetate=20/1 to 1/1) to give tert-butyl 4-[[3-[2-(6-methyl-7-oxo-1H-pyrrolo[2,3-c]pyridin-4-yl)-4-nitro-phenoxy]phenyl]methoxy]piperidine-1-carboxylate (186 mg, 53% yield) as a yellow oil. ¹H NMR (400 MHz, CDCl₃): δ 1.38 (s, 9H), 1.77˜1.79 (m, 2H), 2.31˜2.33 (m, 2H), 2.78 (s, 3H), 2.99-3.06 (m, 2H), 3.48˜3.51 (m, 1H), 3.69˜3.71 (m, 2H), 4.45 (s, 2H), 6.32˜6.35 (m, 1H), 6.86-6.88 (m, 1H), 6.90 (d, J=9.12 Hz, 1H), 6.95 (s, 1H), 7.08-7.11 (m, 2H), 7.20 (s, 1H), 7.25-7.30 (m, 1H), 8.08-8.11 (m, 1H), 8.36 (d, J=2.72 Hz, 1H), 9.66 (brs, 1H).

### Step 4: Synthesis of tert-butyl 4-[[3-[4-amino-2-(6-methyl-7-oxo-1H-pyrrolo[2,3-c]pyridin-4-yl)phenoxy]phenyl]methoxy]piperidine-1-carboxylate

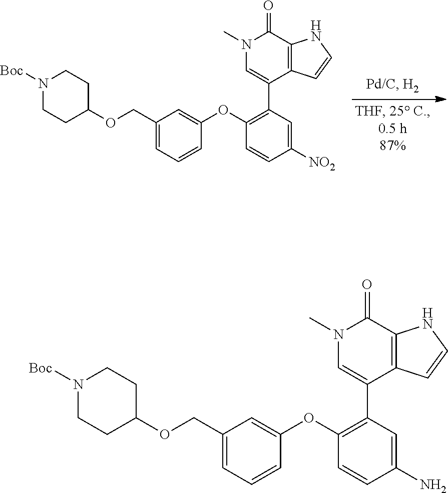 [SMILES: Cn1cc(-c2cc([N+](=O)[O-])ccc2Oc2cccc(COC3CCN(C(=O)OC(C)(C)C)CC3)c2)c2cc[nH]c2c1=O ; Cn1cc(-c2cc(N)ccc2Oc2cccc(COC3CCN(C(=O)OC(C)(C)C)CC3)c2)c2cc[nH]c2c1=O]

To a solution of tert-butyl 4-[[3-[2-(6-methyl-7-oxo-1H-pyrrolo[2,3-c]pyridin-4-yl)-4-nitro-phenoxy]phenyl]methoxy]piperidine-1-carboxylate (80 mg, 139 μmol, 1 equiv) in THF (2 mL) was added Pd/C (10%, 1 g) under N₂ atmosphere. The mixture was stirred under H₂ (15 psi) at 25° C. for 0.5 hour. The mixture was filtered and concentrated to give tert-butyl 4-[[3-[4-amino-2-(6-methyl-7-oxo-1H-pyrrolo[2,3-c]pyridin-4-yl)phenoxy]phenyl]methoxy]piperidine-1-carboxylate (65 mg, 87%) as a yellow oil.

### Step 5: Synthesis of tert-butyl 4-[[3-[4-(ethylsulfonylamino)-2-(6-methyl-7-oxo-1H-pyrrolo[2,3-c]pyridin-4-yl)phenoxy]phenyl]methoxy]piperidine-1-carboxylate

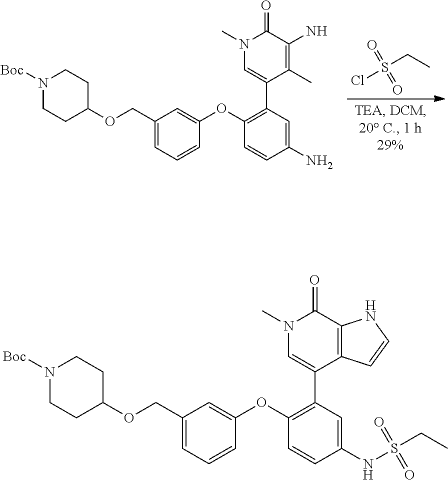

To a solution of tert-butyl 4-[[3-[4-amino-2-(6-methyl-7-oxo-1H-pyrrolo[2,3-c]pyridin-4-yl)phenoxy]phenyl]methoxy]piperidine-1-carboxylate (180 mg, 330 mol, 1 equiv) in DCM (3 mL) was added TEA (334 mg, 3.3 mol, 10 equiv) and ethanesulfonyl chloride (170 mg, 1.32 μmol, 4 equiv). The mixture was stirred at 20° C. for 1 hour. The residue was purified by prep-TLC (SiO₂, DCM/MeOH=20/1) to give tert-butyl 4-[[3-[4-(ethylsulfonylamino)-2-(6-methyl-7-oxo-1H-pyrrolo[2,3-c]pyridin-4-yl)phenoxy]phenyl]methoxy]piperidine-1-carboxylate (60 mg, 29% yield) as a yellow oil.

### Step 6: Synthesis of N-[3-(6-methyl-7-oxo-1H-pyrrolo[2,3-c]pyridin-4-yl)-4-[3-(4-piperidyloxymethyl)phenoxy]phenyl]ethanesulfonamide

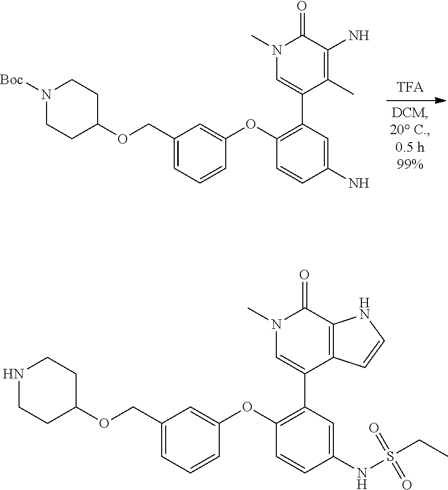 [SMILES: Cc1c(-c2cc(N)ccc2Oc2cccc(COC3CCN(C(=O)OC(C)(C)C)CC3)c2)cn(C)c(=O)c1N ; CCS(=O)(=O)Nc1ccc(Oc2cccc(COC3CCNCC3)c2)c(-c2cn(C)c(=O)c3[nH]ccc23)c1]

To a solution of tert-butyl 4-[[3-[4-(ethylsulfonylamino)-2-(6-methyl-7-oxo-1H-pyrrolo[2,3-c]pyridin-4-yl)phenoxy]phenyl]methoxy]piperidine-1-carboxylate (60 mg, 94 mol, 1 equiv) in DCM (4 mL) was added TFA (2 mL). The mixture was stirred at 20° C. for 0.5 hour and concentrated to give N-[3-(6-methyl-7-oxo-1H-pyrrolo[2,3-c]pyridin-4-yl)-4-[3-(4-piperidyloxymethyl)phenoxy]phenyl]ethanesulfonamide (50 mg) as a yellow oil.

### Step 7: Synthesis of N-((1r, 3r)-3-(3-chloro-4-cyanophenoxy)-2,2,4,4-tetramethylcyclobutyl)-2-(4-((3-(4-(ethylsulfonamido)-2-(6-methyl-7-oxo-6,7-dihydro-1H-pyrrolo[2,3-c]pyridin-4-yl)phenoxy)benzyl)oxy)piperidin-1-yl)pyrimidine-5-carboxamide (I-18)

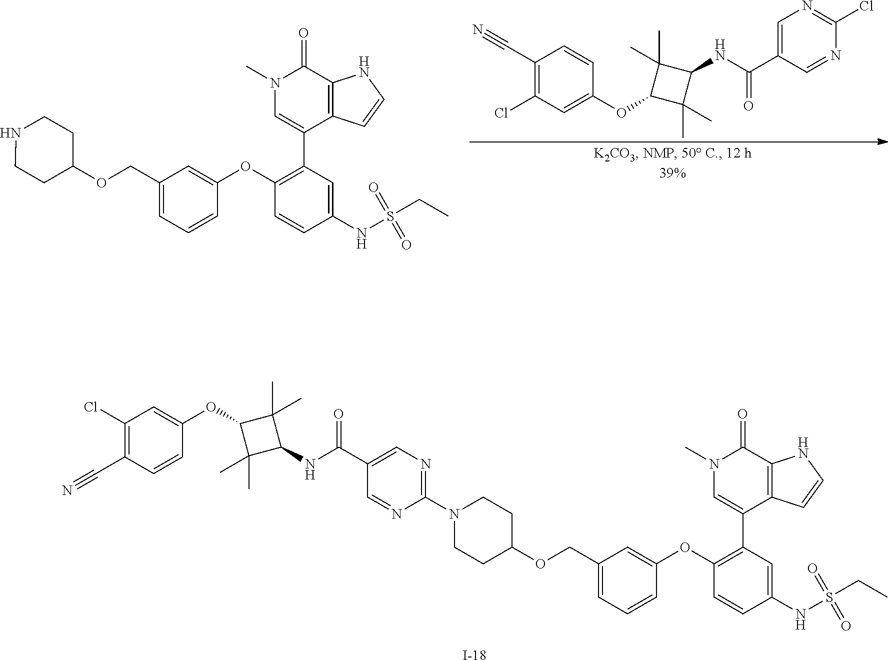

To a solution of N-[3-(6-methyl-7-oxo-1H-pyrrolo[2,3-c]pyridin-4-yl)-4-[3-(4-piperidyloxymethyl)phenoxy]phenyl]ethanesulfonamide (50 mg, 93 μmol, 1 equiv) in NMP (1 mL) was added K₂CO₃ (39 mg, 280 mol, 3 equiv) and 2-chloro-N-[3-(3-chloro-4-cyano-phenoxy)-2,2,4,4-tetramethyl-cyclobutyl]pyrimidine-5-carboxamide (39 mg, 93 mol, 1 equiv). The mixture was stirred at 50° C. for 12 hours. The residue was purified by prep-HPLC (column: Unisil 3-100 C18 Ultra 150 mm×50 mm, 3 μm; mobile phase: [water(FA)-ACN]; B %: 52%-82%, 7 min) to give N-((1r, 3r)-3-(3-chloro-4-cyanophenoxy)-2,2,4,4-tetramethylcyclobutyl)-2-(4-((3-(4-(ethylsulfonamido)-2-(6-methyl-7-oxo-6,7-dihydro-1H-pyrrolo[2,3-c]pyridin-4-yl)phenoxy)benzyl)oxy)piperidin-1-yl)pyrimidine-5-carboxamide (33 mg, 39% yield). ¹H NMR (400 MHz, DMSO-d₆): δ 1.12 (s, 6H), 1.22 (s, 9H), 1.40-1.50 (m, 2H), 1.86˜1.88 (m, 2H), 3.13˜3.15 (m, 2H), 3.47-3.56 (m, 5H), 3.60˜3.61 (m, 1H), 4.04 (d, J=9.04 Hz, 1H), 4.14-4.25 (m, 2H), 4.30 (s, 1H), 4.47 (s, 2H), 6.26 (s, 1H), 6.66-6.89 (m, 2H), 6.94-7.08 (m, 3H), 7.19-7.29 (m, 5H), 7.41 (d, J=2.68 Hz, 1H), 7.72 (d, J=9.28 Hz, 1H), 7.91 (d, J=8.68 Hz, 1H), 8.77 (s, 2H), 9.59-9.93 (m, 1H), 12.04 (s, 1H).

### Example 3—Synthesis of N-((1r, 3r)-3-(3-chloro-4-cyanophenoxy)-2,2,4,4-tetramethylcyclobutyl)-2-(4-((6-(3-(4-(ethylsulfonamido)-2-(6-methyl-7-oxo-6,7-dihydro-1H-pyrrolo[2,3-c]pyridin-4-yl)phenoxy)phenyl)-2,6-diazaspiro[3.3]heptan-2-yl)methyl) piperidin-1-yl)pyrimidine-5-carboxamide (I-35)

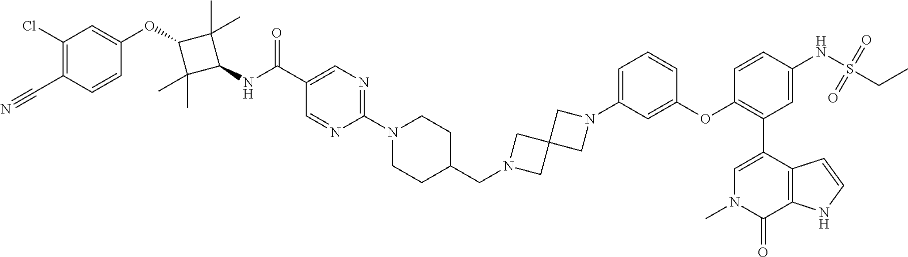 [SMILES: CCS(=O)(=O)Nc1ccc(Oc2cccc(N3CC4(CN(CC5CCN(c6ncc(C(=O)N[C@H]7C(C)(C)[C@H](Oc8ccc(C#N)c(Cl)c8)C7(C)C)cn6)CC5)C4)C3)c2)c(-c2cn(C)c(=O)c3[nH]ccc23)c1]

### Step 1: Synthesis of tert-butyl 6-(3-benzyloxyphenyl)-2,6-diazaspiro[3.3]heptane-2-carboxylate

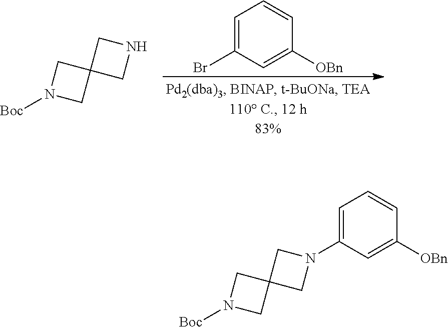 [SMILES: CC(C)(C)OC(=O)N1CC2(CNC2)C1 ; Brc1cccc(OCc2ccccc2)c1 ; CC(C)(C)OC(=O)N1CC2(C1)CN(c1cccc(OCc3ccccc3)c1)C2]

A mixture of tert-butyl 2,6-diazaspiro[3.3]heptane-2-carboxylate (829 mg, 4.2 μmol, 1.1 equiv), 1-benzyloxy-3-bromobenzene (1.0 g, 3.8 μmol, 1.0 equiv), Pd₂(dba)₃ (174 mg, 190 mol, 0.05 equiv), BINAP (236 mg, 380 μmol, 0.1 equiv), NaOtBu (1.2 g, 12.5 mol, 3.3 equiv), and TEA (423 mg, 4.2 μmol, 1.1 equiv) in toluene (25 mL) was degassed and purged with N₂ three times. The mixture was stirred at 1100 C. for 12 hours under N₂ atmosphere. The residue was purified by column chromatography (SiO₂, petroleum ether/ethyl acetate=10/1 to 3/1) to give tert-butyl 6-(3-benzyloxyphenyl)-2,6-diazaspiro [3.3]heptane-2-carboxylate (1.2 g, 83% yield) as a yellow solid. ¹H NMR (400 MHz, CDCl₃): δ 7.51-7.31 (m, 5H), 7.19-7.09 (m, 1H), 6.48-6.38 (m, 1H), 6.20-6.05 (m, 2H), 5.15-4.98 (m, 2H), 4.14-4.06 (m, 4H), 4.01-3.93 (m, 4H), 1.47 (s, 9H).

### Step 2: Synthesis of 2-(3-benzyloxyphenyl)-2,6-diazaspiro[3.3]heptane

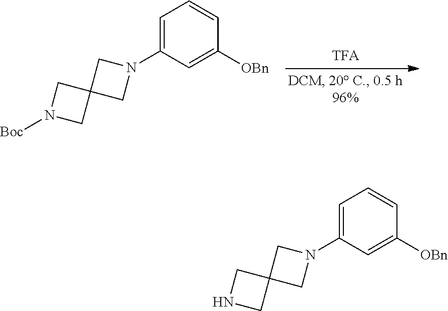 [SMILES: CC(C)(C)OC(=O)N1CC2(C1)CN(c1cccc(OCc3ccccc3)c1)C2 ; c1ccc(COc2cccc(N3CC4(CNC4)C3)c2)cc1]

To a solution of tert-butyl 6-(3-benzyloxyphenyl)-2,6-diazaspiro[3.3]heptane-2-carboxylate (500 mg, 1.3 mol, 1.0 equiv) in DCM (5 mL) was added TFA (0.5 mL). The mixture was stirred at 20° C. for 0.5 hour. The mixture was concentrated to give 2-(3-benzyloxyphenyl)-2,6-diazaspiro[3.3]heptane (500 mg, 96% yield, TFA salt) as a yellow gum.

### Step 3: Synthesis of tert-butyl 4-[[2-(3-benzyloxyphenyl)-2,6-diazaspiro[3.3]heptan-6-yl]methyl]piperidine-1-carboxylate

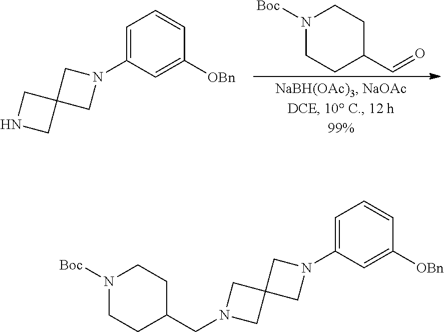 [SMILES: c1ccc(COc2cccc(N3CC4(CNC4)C3)c2)cc1 ; CC(C)(C)OC(=O)N1CCC(C=O)CC1 ; CC(C)(C)OC(=O)N1CCC(CN2CC3(C2)CN(c2cccc(OCc4ccccc4)c2)C3)CC1]

To a solution of 2-(3-benzyloxyphenyl)-2,6-diazaspiro[3.3]heptane (500 mg, 1.27 mol, 1 equiv, TFA salt) in DCE (20 mL) was added NaOAc (520 mg, 6.34 mol, 5.0 equiv), tert-butyl 4-formylpiperidine-1-carboxylate (297 mg, 1.4 μmol, 1.1 equiv), and NaBH(OAc)₃ (806 mg, 3.8 mol, 3.0 equiv). The mixture was stirred at 100 C. for 12 hours. The aqueous phase was extracted with DCM (3×50 mL). The combined organic phase was washed with brine (30 mL), dried with anhydrous Na₂SO₄, filtered, and concentrated. The residue was purified by column chromatography (SiO₂, petroleum ether/ethyl acetate=10/1 to 0/1) to give tert-butyl 4-[[2-(3-benzyloxyphenyl)-2,6-diazaspiro[3.3]heptan-6-yl]methyl]piperidine-1-carboxylate (600 mg, 99% yield) as a yellow gum.

### Step 4: Synthesis of tert-butyl 4-[[2-(3-hydroxyphenyl)-2,6-diazaspiro[3.3]heptan-6-yl]methyl]piperidine-1-carboxylate

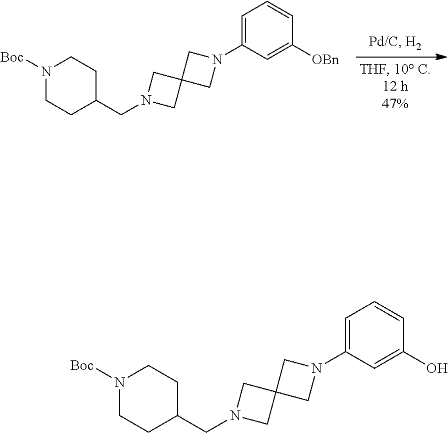 [SMILES: CC(C)(C)OC(=O)N1CCC(CN2CC3(C2)CN(c2cccc(OCc4ccccc4)c2)C3)CC1 ; CC(C)(C)OC(=O)N1CCC(CN2CC3(C2)CN(c2cccc(O)c2)C3)CC1]

To a solution of tert-butyl 4-[[2-(3-benzyloxyphenyl)-2,6-diazaspiro[3.3]heptan-6-yl]methyl]piperidine-1-carboxylate (450 mg, 942 mol, 1.0 equiv) in THF (10 mL) was added Pd/C (60 mg, 10% purity). The mixture was stirred under H₂ (15 psi) at 100 C. for 12 hours. The reaction mixture was filtered and concentrated. The residue was purified by prep-TLC (SiO₂, DCM/MeOH=10/1) to give tert-butyl 4-[[2-(3-hydroxyphenyl)-2,6-diazaspiro [3.3]heptan-6-yl]methyl]piperidine-1-carboxylate (170 mg, 47% yield) as a colorless gum.

### Step 5: Synthesis of tert-butyl 4-[[2-[3-[2-[6-methyl-7-oxo-1-(p-tolylsulfonyl) pyrrolo[2,3-c]pyridin-4-yl]-4-nitro-phenoxy]phenyl]-2,6-diazaspiro[3.3]heptan-6-yl]methyl]piperidine-1-carboxylate

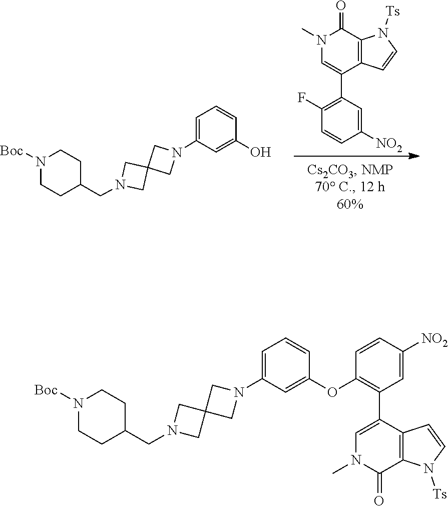 [SMILES: CC(C)(C)OC(=O)N1CCC(CN2CC3(C2)CN(c2cccc(O)c2)C3)CC1 ; Cc1ccc(S(=O)(=O)n2ccc3c(-c4cc([N+](=O)[O-])ccc4F)cn(C)c(=O)c32)cc1 ; Cc1ccc(S(=O)(=O)n2ccc3c(-c4cc([N+](=O)[O-])ccc4Oc4cccc(N5CC6(CN(CC7CCN(C(=O)OC(C)(C)C)CC7)C6)C5)c4)cn(C)c(=O)c32)cc1]

To a solution of tert-butyl 4-[[2-(3-hydroxyphenyl)-2,6-diazaspiro[3.3]heptan-6-yl]methyl]piperidine-1-carboxylate (120 mg, 310 μmol, 1.0 equiv) in NMP (4 mL) was added Cs₂CO₃ (302 mg, 929 mol, 3.0 equiv) and 4-(2-fluoro-5-nitro-phenyl)-6-methyl-1-(p-tolylsulfonyl)pyrrolo[2,3-c]pyridin-7-one (136 mg, 310 μmol, 1.0 equiv). The mixture was stirred at 70° C. for 12 hours. The aqueous phase was extracted with ethyl acetate (3×50 mL). The combined organic phase was washed with brine (50 mL×2), dried with anhydrous Na₂SO₄, filtered, and concentrated. The residue was purified by prep-TLC (DCM/MeOH=10/1) to give tert-butyl 4-[[2-[3-[2-[6-methyl-7-oxo-1-(p-tolylsulfonyl)pyrrolo[2,3-c]pyridin-4-yl]-4-nitro-phenoxy]phenyl]-2,6-diazaspiro[3.3]heptan-6-yl]methyl]piperidine-1-carboxylate (150 mg, 60% yield) as a yellow gum.

### Step 6: Synthesis of tert-butyl 4-[[2-[3-[4-amino-2-[6-methyl-7-oxo-1-(p-tolylsulfonyl)pyrrolo[2,3-c]pyridin-4-yl]phenoxy]phenyl]-2,6-diazaspiro[3.3]heptan-6-yl]methyl]piperidine-1-carboxylate

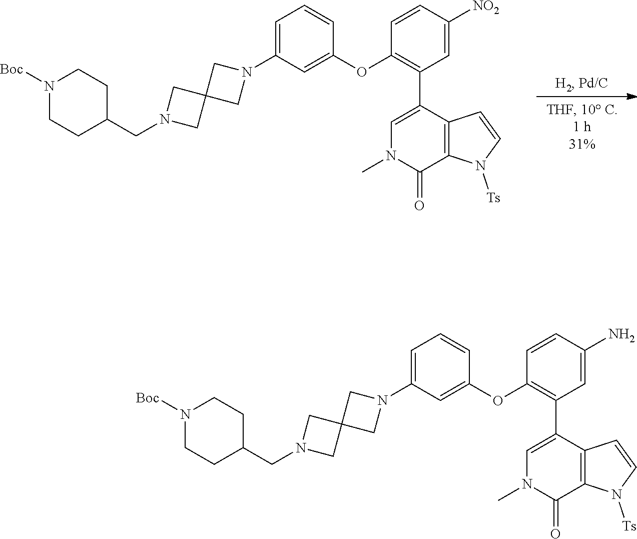 [SMILES: Cc1ccc(S(=O)(=O)n2ccc3c(-c4cc([N+](=O)[O-])ccc4Oc4cccc(N5CC6(CN(CC7CCN(C(=O)OC(C)(C)C)CC7)C6)C5)c4)cn(C)c(=O)c32)cc1 ; Cc1ccc(S(=O)(=O)n2ccc3c(-c4cc(N)ccc4Oc4cccc(N5CC6(CN(CC7CCN(C(=O)OC(C)(C)C)CC7)C6)C5)c4)cn(C)c(=O)c32)cc1]

To a solution of tert-butyl 4-[[2-[3-[2-[6-methyl-7-oxo-1-(p-tolylsulfonyl)pyrrolo [2,3-c]pyridin-4-yl]-4-nitro-phenoxy]phenyl]-2,6-diazaspiro[3.3]heptan-6-yl]methyl]piperidine-1-carboxylate (200 mg, 247 mol, 1.0 equiv) in THE (10 mL) was added Pd/C (60 mg, 10% purity). The mixture was stirred under H₂ (15 psi) at 100 C. for 1 hour. The mixture was filtered, and the filter was concentrated. The residue was purified by prep-TLC (SiO₂, DCM/MeOH=10/1) to give tert-butyl 4-[[2-[3-[4-amino-2-[6-methyl-7-oxo-1-(p-tolylsulfonyl)pyrrolo[2,3-c]pyridin-4-yl]phenoxy]phenyl]-2,6-diazaspiro[3.3]heptan-6-yl]methyl]piperidine-1-carboxylate (60 mg, 31% yield) as a yellow gum.

### Step 7: Synthesis of tert-butyl 4-[[2-[3-[4-(ethylsulfonylamino)-2-[6-methyl-7-oxo-1-(p-tolylsulfonyl)pyrrolo[2,3-c]pyridin-4-yl]phenoxy]phenyl]-2,6-diazaspiro [3.3]heptan-6-yl]methyl]piperidine-1-carboxylate

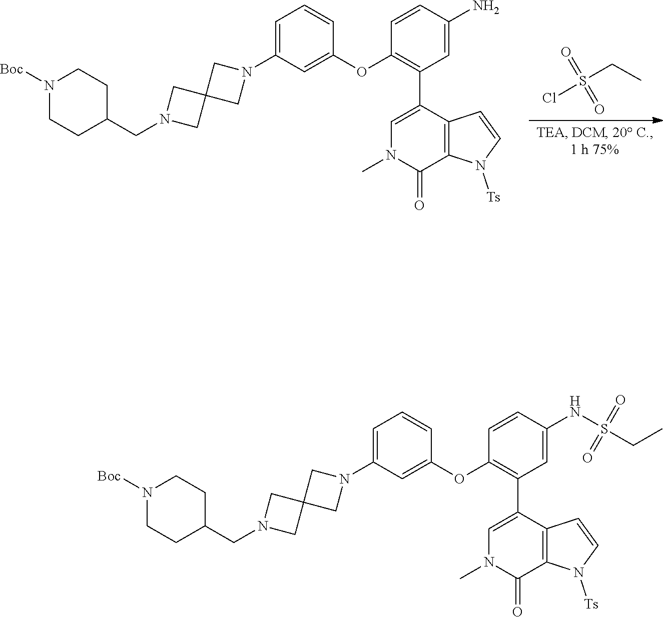

To a solution of tert-butyl 4-[[2-[3-[4-amino-2-[6-methyl-7-oxo-1-(p-tolylsulfonyl)p yrrolo[2,3-c]pyridin-4-yl]phenoxy]phenyl]-2,6-diazaspiro[3.3]heptan-6-yl]methyl]piperidine-1-carboxylate (60 mg, 77 mol, 1.0 equiv) in DCM (2 mL) was added TEA (39 mg, 385 mol, 5.0 equiv) and ethanesulfonyl chloride (20 mg, 154 mol, 2.0 equiv). The mixture was stirred at 20° C. for 1 hour. The residue was purified by prep-TLC (DCM/MeOH=10/1) to give tert-butyl4-[[2-[3-[4-(ethylsulfonylamino)-2-[6-methyl-7-oxo-1-(p-tolylsulfonyl) pyrrolo[2,3-c]pyridin-4-yl]phenoxy]phenyl]-2,6-diazaspiro[3.3]heptan-6-yl]methyl]piperidine-1-carboxylate (50 mg, 75% yield) as a yellow gum.

### Step 8: Synthesis of tert-butyl 4-[[2-[3-[4-(ethylsulfonylamino)-2-(6-methyl-7-oxo-1H-pyrrolo[2,3-c]pyridin-4-yl)phenoxy]phenyl]-2,6-diazaspiro[3.3]heptan-6-yl]methyl]piperidine-1-carboxylate

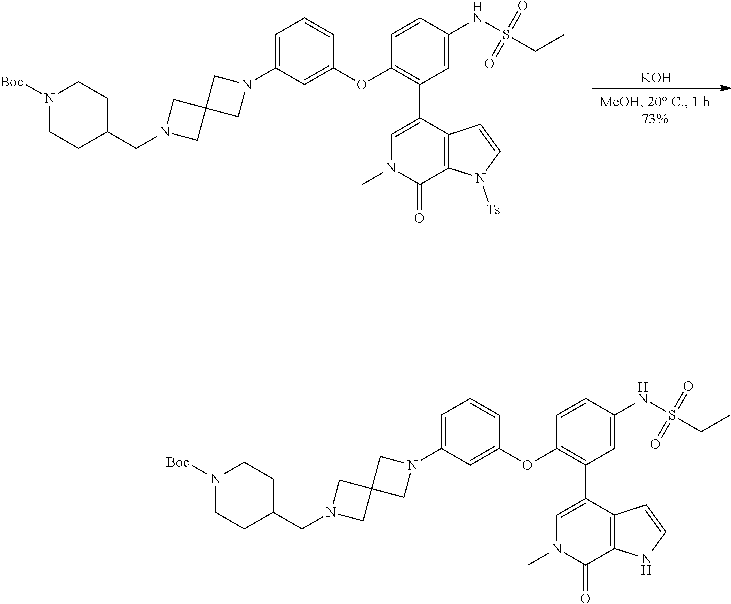 [SMILES: CCS(=O)(=O)Nc1ccc(Oc2cccc(N3CC4(CN(CC5CCN(C(=O)OC(C)(C)C)CC5)C4)C3)c2)c(-c2cn(C)c(=O)c3c2ccn3S(=O)(=O)c2ccc(C)cc2)c1 ; "KOH" ; CCS(=O)(=O)Nc1ccc(Oc2cccc(N3CC4(CN(CC5CCN(C(=O)OC(C)(C)C)CC5)C4)C3)c2)c(-c2cn(C)c(=O)c3[nH]ccc23)c1]

To a solution of tert-butyl 4-[[2-[3-[4-(ethylsulfonylamino)-2-[6-methyl-7-oxo-1-(p-tolylsulfonyl)pyrrolo[2,3-c]pyridin-4-yl]phenoxy]phenyl]-2,6-diazaspiro[3.3]heptan-6-yl]methyl]piperidine-1-carboxylate (50 mg, 57 μmol, 1.0 equiv) in MeOH (3 mL) was added KOH (64 mg, 1.2 mol, 20 equiv). The mixture was stirred at 20° C. for 1 hour and purified by prep-TLC (SiO₂, DCM/MeOH=10/1) to give tert-butyl 4-[[2-[3-[4-(ethylsulfonylamino)-2-(6-methyl-7-oxo-1H-pyrrolo[2,3-c]pyridin-4-yl)phenoxy]phenyl]-2,6-diazaspiro[3.3]heptan-6-yl]methyl]piperidine-1-carboxylate (30 mg, 73% yield) as a yellow gum.

### Step 9: Synthesis of N-[3-(6-methyl-7-oxo-1H-pyrrolo[2,3-c]pyridin-4-yl)-4-[3-[6-(4-piperidylmethyl)-2,6-diazaspiro[3.3]heptan-2-yl]phenoxy]phenyl]ethane-sulfonamide

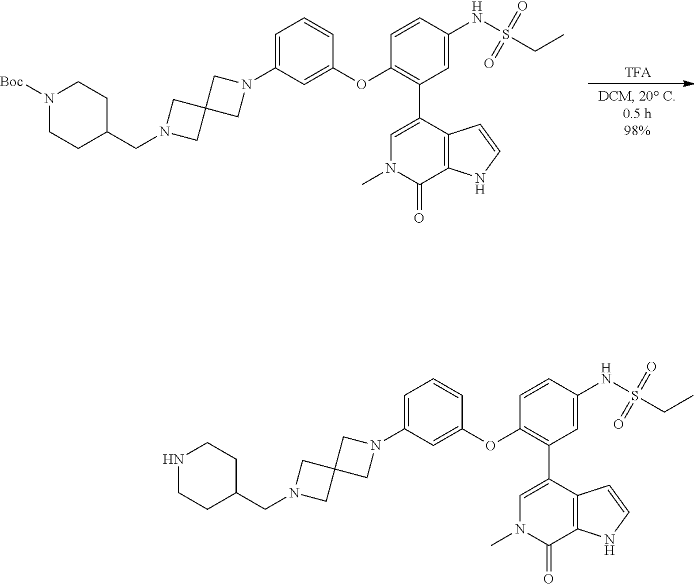 [SMILES: CCS(=O)(=O)Nc1ccc(Oc2cccc(N3CC4(CN(CC5CCN(C(=O)OC(C)(C)C)CC5)C4)C3)c2)c(-c2cn(C)c(=O)c3[nH]ccc23)c1 ; CCS(=O)(=O)Nc1ccc(Oc2cccc(N3CC4(CN(CC5CCNCC5)C4)C3)c2)c(-c2cn(C)c(=O)c3[nH]ccc23)c1]

To a solution of tert-butyl 4-[[2-[3-[4-(ethylsulfonylamino)-2-(6-methyl-7-oxo-1H-pyrrolo[2,3-c]pyridin-4-yl)phenoxy]phenyl]-2,6-diazaspiro[3.3]heptan-6-yl]methyl]piperidine-1-carboxylate (30 mg, 42 mol, 1.0 equiv) in DCM (5 mL) was added TFA (500 μL). The mixture was stirred at 20° C. for 0.5 hour and concentrated to give N-[3-(6-methyl-7-oxo-1H-pyrrolo[2,3-c]pyridin-4-yl)-4-[3-[6-(4-piperidylmethyl)-2,6-diazaspiro[3.3]heptan-2-yl]phenoxy]phenyl]ethanesulfonamide (30 mg, TFA salt) as a yellow gum.

### Step 10: Synthesis of N-((1r, 3r)-3-(3-chloro-4-cyanophenoxy)-2,2,4,4-tetramethylcyclobutyl)-2-(4-((6-(3-(4-(ethylsulfonamido)-2-(6-methyl-7-oxo-6,7-dihydro-1H-pyrrolo[2,3-c]pyridin-4-yl)phenoxy)phenyl)-2,6-diazaspiro[3.3]heptan-2-yl)methyl)piperidin-1-yl)pyrimidine-5-carboxamide (I-35)

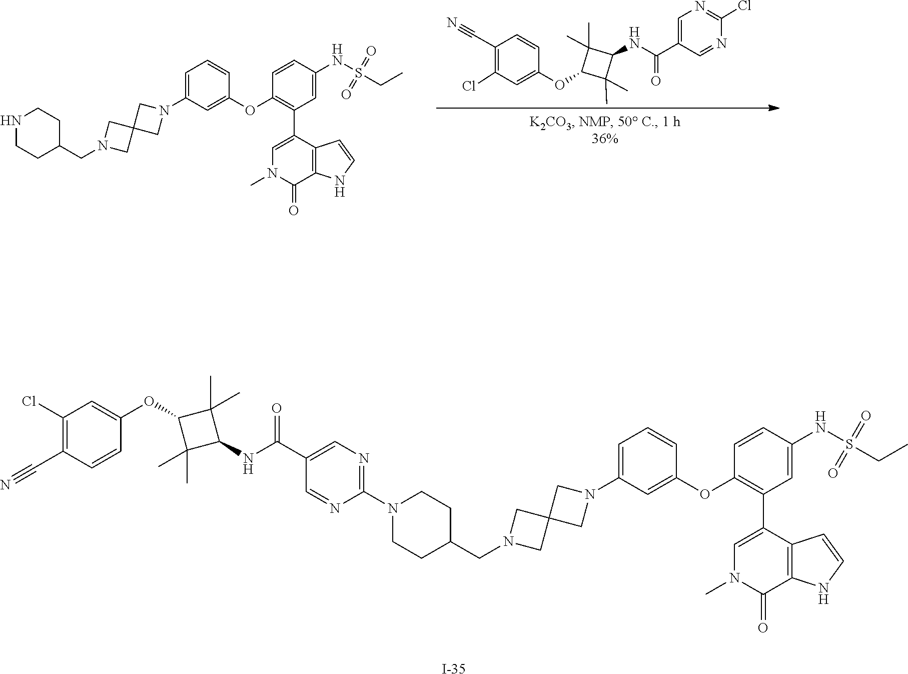

To a solution of N-[3-(6-methyl-7-oxo-1H-pyrrolo[2,3-c]pyridin-4-yl)-4-[3-[6-(4-piperidylmethyl)-2,6-diazaspiro[3.3]heptan-2-yl]phenoxy]phenyl]ethanesulfonamide (30 mg, 41 μmol, 1.0 equiv, TFA salt) in NMP (3 mL) was added K₂CO₃ (17 mg, 123 μmol, 3.0 equiv) and tert-butyl N-[3-[5-(2,5-difluorophenyl)-3-[methyl-[2-[2-[2-[2-[2-(4-piperidyloxy)ethoxy]ethoxy]ethoxy]ethoxy]ethyl]carbamoyl]-2-phenyl-1,3,4-thiadiazol-2-yl]propyl]carbamate (17 mg, 41 μmol, 1.0 equiv). The mixture was stirred at 50° C. for 1 hour. The residue was purified by prep-HPLC (column: Unisil 3-100 C18 Ultra 150 mm×50 mm, 3 μm; mobile phase: [water(FA)-ACN]; B %: 26%-56%, 7 min and column: Waters Xbridge 150 mm×25 mm, 5 μm; mobile phase: [water(NH₄HCO₃)-ACN]; B %: 55%-85%, 9 min) to give N-((1r, 3r)-3-(3-chloro-4-cyanophenoxy)-2,2,4,4-tetramethylcyclobutyl)-2-(4-((6-(3-(4-(ethylsulfonamido)-2-(6-methyl-7-oxo-6,7-dihydro-1H-pyrrolo[2,3-c]pyridin-4-yl)phenoxy) phenyl)-2,6-diazaspiro[3.3]heptan-2-yl)methyl)piperidin-1-yl)pyrimidine-5-carboxamide (15 mg, 36% yield) as a white solid. ¹H NMR (400 MHz, DMSO-d₆): δ 12.05 (s, 1H), 9.86-9.74 (m, 1H), 8.78-8.74 (m, 2H), 7.91 (d, J=8.8 Hz, 1H), 7.78-7.65 (m, 1H), 7.41-7.37 (m, 1H), 7.31-7.20 (m, 4H), 7.05-7.00 (m, 3H), 6.21 (s, 1H), 6.18-6.01 (m, 2H), 5.90-5.85 (m, 1H), 4.76-4.69 (m, 2H), 4.32-4.28 (m, 1H), 4.06-4.02 (m, 1H), 3.77 (s, 4H), 3.50 (s, 3H), 3.40 (s, 2H), 3.30-3.24 (m, 2H), 3.13 (q, J=7.2 Hz, 2H), 2.95 (t, J=12.0 Hz, 2H), 1.75 (d, J=11.2 Hz, 2H), 1.66-1.56 (m, 1H), 1.27-1.21 (m, 10H), 1.16-1.01 (m, 9H).

### Example 4—Synthesis of N-((1r, 3r)-3-(3-chloro-4-cyanophenoxy)-2,2,4,4-tetra-methylcyclobutyl)-2-(4-(2-((6-(4-(ethylsulfonamido)-2-(6-methyl-7-oxo-6,7-dihydro-1H-pyrrolo[2,3-c]pyridin-4-yl)phenoxy)pyridin-2-yl)oxy)ethoxy)piperidin-1-yl)pyrimidine-5-carboxamide (1-47)

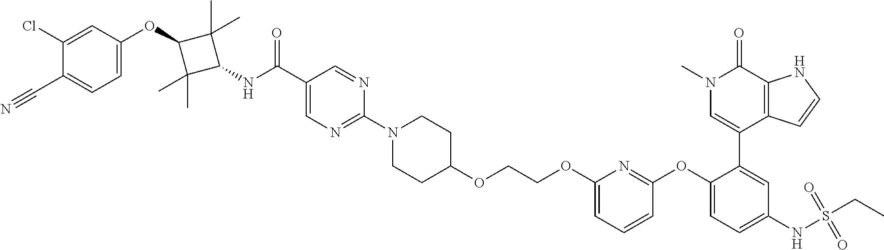 [SMILES: CCS(=O)(=O)Nc1ccc(Oc2cccc(OCCOC3CCN(c4ncc(C(=O)N[C@H]5C(C)(C)[C@H](Oc6ccc(C#N)c(Cl)c6)C5(C)C)cn4)CC3)n2)c(-c2cn(C)c(=O)c3[nH]ccc23)c1]

### 1.1 Synthesis of Compound 2

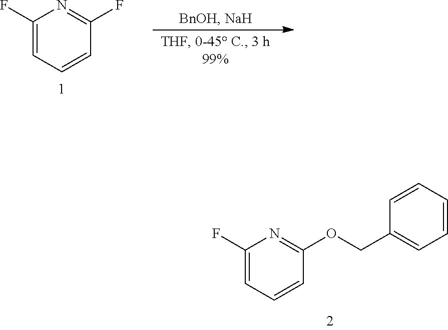 [SMILES: Fc1cccc(F)n1 ; Fc1cccc(OCc2ccccc2)n1]

To a solution of phenylmethanol (1.5 g, 14 mol, 1.0 equiv) in THF (30 mL) was added NaH (666 mg, 17 μmol, 60% purity, 1.2 equiv) at 0° C. The mixture was stirred at 45° C. for 1 h. Then 2,6-difluoropyridine (1.6 g, 14 μmol, 1.0 equiv) was added, the mixture was stirred at 20° C. for 2 h. The reaction quench by NH₄Cl (15 mL) and the mixture was concentrated. The residue was purified by column chromatography (SiO₂, Petroleum ether/Ethyl acetate=50/1 to 3/1) to give 2-benzyloxy-6-fluoro-pyridine (2.8 g, 99% yield) as a yellow oil. ¹H NMR (400 MHz, CDCl₃): δ 7.68 (q, J=8.0 Hz, 1H), 7.52-7.46 (m, 2H), 7.44-7.34 (m, 3H), 6.72-6.66 (m, 1H), 6.55-6.49 (m, 1H), 5.37 (s, 2H).

### 1.2 Synthesis of Compound 4

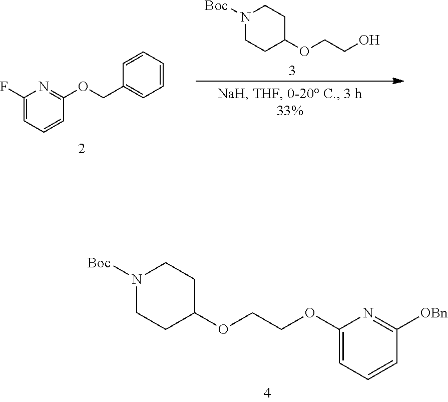 [SMILES: CC(C)(C)OC(=O)N1CCC(OCCO)CC1 ; Fc1cccc(OCc2ccccc2)n1 ; CC(C)(C)OC(=O)N1CCC(OCCOc2cccc(OCc3ccccc3)n2)CC1]

To a solution of tert-butyl 4-(2-hydroxyethoxy)piperidine-1-carboxylate (603 mg, 2.46 mol, 1.0 equiv) in THF (15 mL) was added NaH (118 mg, 2.95 μmol, 60% purity, 1.2 equiv) at 0° C. The mixture was stirred at 20° C. for 1 h. Then 2-benzyloxy-6-fluoro-pyridine (500 mg, 2.46 mol, 1.0 equiv) was added and the mixture was stirred at 20° C. for 2 h. The reaction was quenched by NH₄Cl (15 mL) and the mixture was concentrated. The residue was purified by column chromatography (SiO₂, Petroleum ether/Ethyl acetate=10/1 to 3/1) to give tert-butyl 4-[2-[(6-benzyloxy-2-pyridyl)oxy]ethoxy]piperidine-1-carboxylate (350 mg, 33% yield) as a colorless oil. ¹H NMR (400 MHz, CDC₃): δ 7.44-7.42 (m, 1H), 7.40-7.31 (m, 2H), 7.29-7.27 (m, 2H), 7.24-7.23 (m, 1H), 6.38-6.25 (m, 2H), 5.24 (s, 2H), 4.40-4.38 (m, 2H), 3.70-3.67 (m, 2H), 3.48-3.35 (m, 1H), 3.03-2.96 (m, 2H), 1.77-1.74 (m, 2H), 1.46-1.42 (m, 2H), 1.38 (s, 9H).

### 1.3 Synthesis of Compound 5

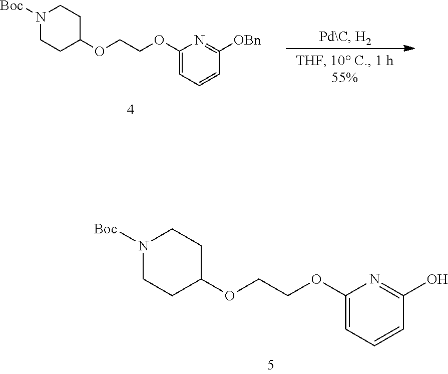 [SMILES: CC(C)(C)OC(=O)N1CCC(OCCOc2cccc(OCc3ccccc3)n2)CC1 ; CC(C)(C)OC(=O)N1CCC(OCCOc2cccc(O)n2)CC1]

To a solution of tert-butyl 4-[2-[(6-benzyloxy-2-pyridyl)oxy]ethoxy]pipendine-1-carboxylate (300 mg, 700 mol, 1.0 equiv) in THF (10 mL) was added Pd/C (60 mg, 10% purity). The mixture was stirred under H₂ (15 psi) at 100 C. for 1 h. The mixture was filtered and the filter was concentrated. The residue was purified by prep-TLC (SiO₂, Petroleum ether/Ethyl acetate=3/1) to give the desired product tert-butyl 4-[2-[(6-hydroxy-2-pyridyl) oxy]ethoxy]piperidine-1-carboxylate (130 mg, 55% yield) as a colorless gum.

### 1.4 Synthesis of Compound 7

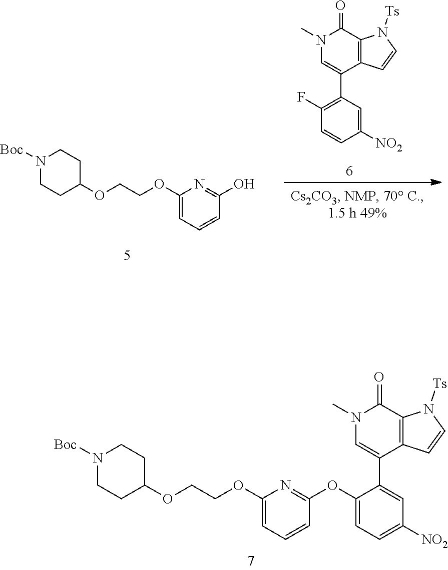 [SMILES: Cc1ccc(S(=O)(=O)n2ccc3c(-c4cc([N+](=O)[O-])ccc4F)cn(C)c(=O)c32)cc1 ; CC(C)(C)OC(=O)N1CCC(OCCOc2cccc(O)n2)CC1 ; Cc1ccc(S(=O)(=O)n2ccc3c(-c4cc([N+](=O)[O-])ccc4Oc4cccc(OCCOC5CCN(C(=O)OC(C)(C)C)CC5)n4)cn(C)c(=O)c32)cc1]

To a solution of tert-butyl 4-[2-[(6-hydroxy-2-pyridyl)oxy]ethoxy]piperidine-1-carboxylate (100 mg, 295 mol, 1.0 equiv) in NMP (3 mL) was added Cs₂CO₃ (289 mg, 886 mol, 3.0 equiv) and 4-(2-fluoro-5-nitro-phenyl)-6-methyl-1-(ptolylsulfonyl)pyrrolo[2,3-c]pyridin-7-one (130 mg, 295 mol, 1.0 equiv). The mixture was stirred at 70° C. for 1.5 h. The aqueous phase was extracted with EA (3×50 mL). The combined organic phase was washed with brine (30 mL), dried with anhydrous Na₂SO₄, filtered and concentrated. The residue was purified by prep-TLC (SiO₂, DCM/MeOH=10/1) to give tert-butyl 4-[2-[[6-[2-[6-methyl-7-oxo-1-(p-tolylsulfonyl)pyrrolo[2,3-c]pyridin-4-yl]-4-nitro-phenoxy]-2-pyridyl]oxy]ethoxy]piperidine-1-carboxylate (110 mg, 49% yield) as a yellow gum.

### 1.5 Synthesis of Compound 8

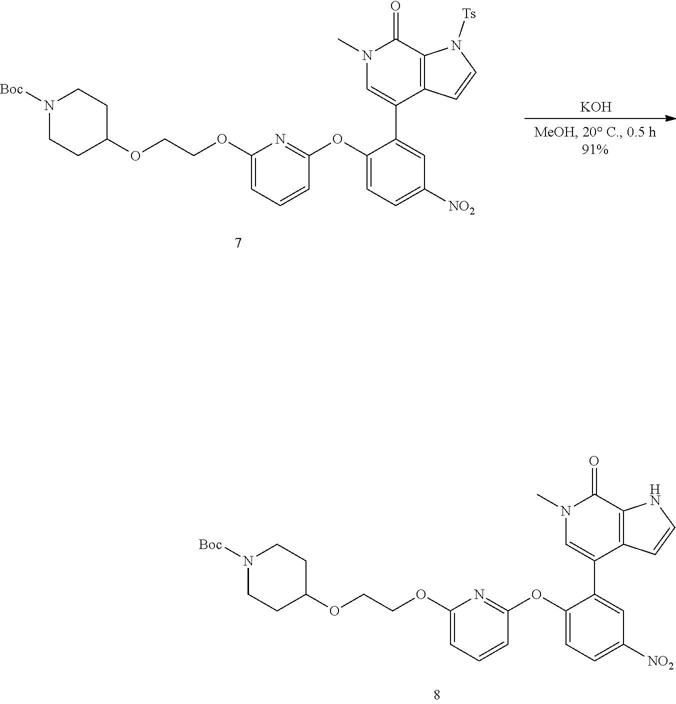 [SMILES: Cc1ccc(S(=O)(=O)n2ccc3c(-c4cc([N+](=O)[O-])ccc4Oc4cccc(OCCOC5CCN(C(=O)OC(C)(C)C)CC5)n4)cn(C)c(=O)c32)cc1 ; "KOH" ; Cn1cc(-c2cc([N+](=O)[O-])ccc2Oc2cccc(OCCOC3CCN(C(=O)OC(C)(C)C)CC3)n2)c2cc[nH]c2c1=O]

To a solution of tert-butyl 4-[2-[[6-[2-[6-methyl-7-oxo-1-(p-tolylsulfonyl) pyrrolo[2,3-c]pyridin-4-yl]-4-nitro-phenoxy]-2-pyridyl]oxy]ethoxy]piperidine-1-carboxylate (110 mg, 145 μmol, 1.0 equiv) in MeOH (3 mL) was added KOH (162 mg, 2.9 μmol, 20 equiv), the mixture was stirred at 20° C. for 0.5 h. The residue was purified by prep-TLC (DCM/MeOH=10/1) to give tert-butyl 4-[2-[[6-[2-(6-methyl-7-oxo-1H-pyrrolo[2,3-c]pyridin-4-yl)-4-nitro-phenoxy]-2-pyridyl]oxy]ethoxy]piperidine-1-carboxylate (80 mg, 91% yield) as a yellow gum.

### 1.6 Synthesis of Compound 9

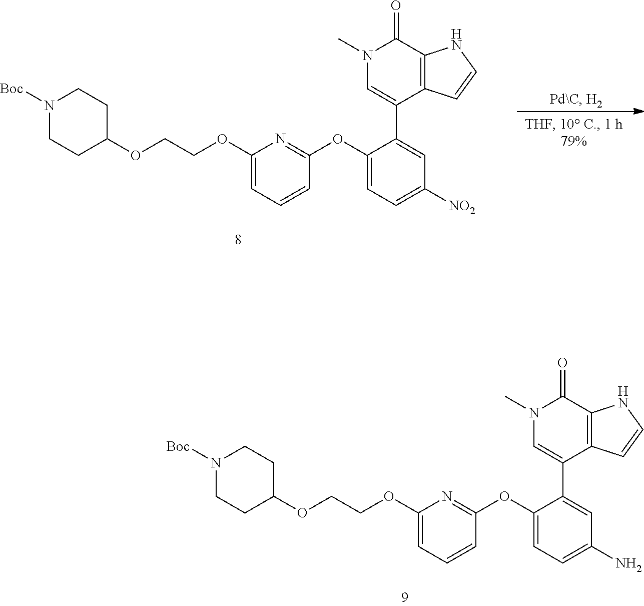 [SMILES: Cn1cc(-c2cc([N+](=O)[O-])ccc2Oc2cccc(OCCOC3CCN(C(=O)OC(C)(C)C)CC3)n2)c2cc[nH]c2c1=O ; Cn1cc(-c2cc(N)ccc2Oc2cccc(OCCOC3CCN(C(=O)OC(C)(C)C)CC3)n2)c2cc[nH]c2c1=O]

To a solution of tert-butyl 4-[2-[[6-[2-(6-methyl-7-oxo-1H-pyrrolo[2,3-c]pyridin-4-yl)-4-nitro-phenoxy]-2-pyridyl]oxy]ethoxy]piperidine-1-carboxylate (80 mg, 132 μmol, 1.0 equiv) in THF (20 mL) was added Pd/C (50 mg, 10% purity). The mixture was stirred under H₂ (15 psi) at 100 C. for 1 h. The mixture was filtered and the filter was concentrated to give tert-butyl 4-[2-[[6-[4-amino-2-(6-methyl-7-oxo-1H-pyrrolo[2,3-c]pyridin-4-yl)phenoxy]-2-pyridyl]oxy]ethoxy]piperidine-1-carboxylate (60 mg, 79% yield) as a colorless gum.

### 1.7 Synthesis of Compound 10

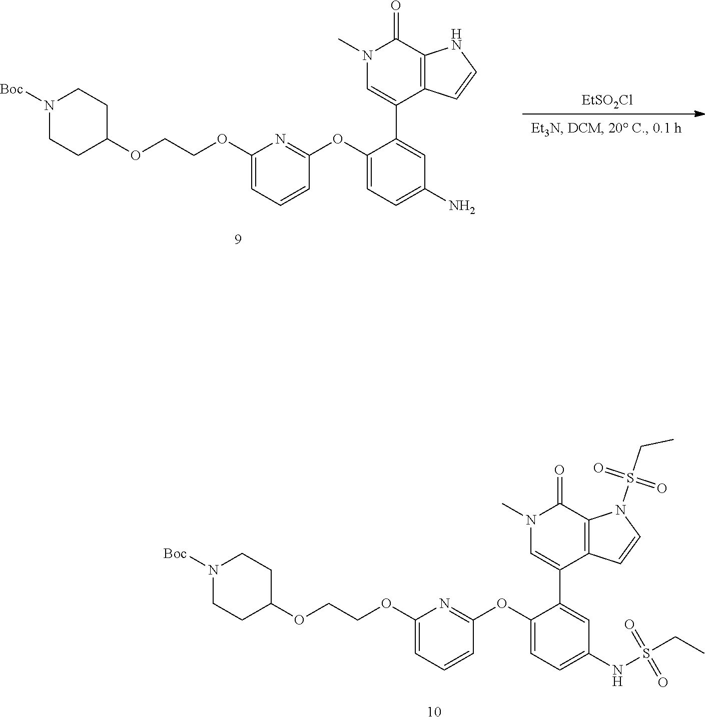 [SMILES: "EtSO2Cl" ; Cn1cc(-c2cc(N)ccc2Oc2cccc(OCCOC3CCN(C(=O)OC(C)(C)C)CC3)n2)c2cc[nH]c2c1=O ; CCS(=O)(=O)Nc1ccc(Oc2cccc(OCCOC3CCN(C(=O)OC(C)(C)C)CC3)n2)c(-c2cn(C)c(=O)c3c2ccn3S(=O)(=O)CC)c1]

To a solution of tert-butyl 4-[2-[[6-[4-amino-2-(6-methyl-7-oxo-1H-pyrrolo[2,3-c]pyridin-4-yl)phenoxy]-2-pyridyl]oxy]ethoxy]piperidine-1-carboxylate (100 mg, 173 mol, 1.0 equiv) in DCM (3 mL) was added TEA (88 mg, 868 μmol, 121 μL, 5 equiv) and ethanesulfonyl chloride (45 mg, 347 μmol, 2.0 equiv). The mixture was stirred at 20° C. for 0.1 h. The mixture was concentrated to give tert-butyl 4-[2-[[6-[4-(ethylsulfonylamino)-2-(1-ethylsulfonyl-6-methyl-7-oxo-pyrrolo[2,3-c]pyridin-4-yl)phenoxy]-2-pyridyl]oxy]ethoxy]piperidine-1-carboxylate (130 mg, crude) as a yellow gum.

### 1.8 Synthesis of Compound 11

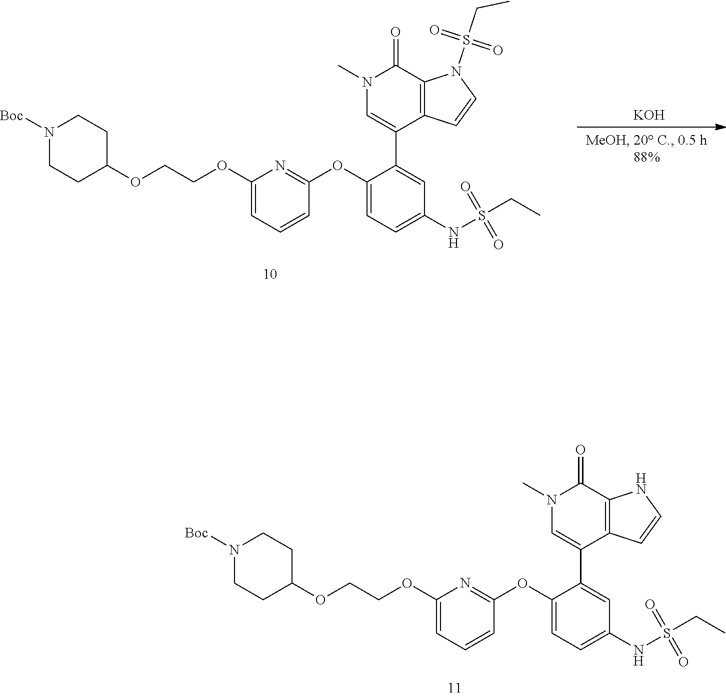 [SMILES: CCS(=O)(=O)Nc1ccc(Oc2cccc(OCCOC3CCN(C(=O)OC(C)(C)C)CC3)n2)c(-c2cn(C)c(=O)c3c2ccn3S(=O)(=O)CC)c1 ; "KOH" ; CCS(=O)(=O)Nc1ccc(Oc2cccc(OCCOC3CCN(C(=O)OC(C)(C)C)CC3)n2)c(-c2cn(C)c(=O)c3[nH]ccc23)c1]

To a solution of tert-butyl 4-[2-[[6-[4-(ethylsulfonylamino)-2-(1-ethylsulfonyl-6-methyl-7-oxo-pyrrolo[2,3-c]pyridin-4-yl)phenoxy]-2-pyridyl]oxy]ethoxy]piperidine-1-carboxylate (130 mg, 171 mol, 1.0 equiv) in MeOH (3 mL) was added KOH (96 mg, 1.71 μmol, 10.0 equiv). The mixture was stirred at 20° C. for 0.5 h. The residue was purified by prep-TLC (SiO₂, DCM/MeOH=10/1) to give the desired product tert-butyl 4-[2-[[6-[4-(ethylsulfonylamino)-2-(6-methyl-7-oxo-1H-pyrrolo[2,3-c]pyridin-4-yl)phenoxy]-2-pyridyl]oxy]ethoxy]piperidine-1-carboxylate (100 mg, 88% yield) as yellow gum.

### 1.9 Synthesis of Compound 12

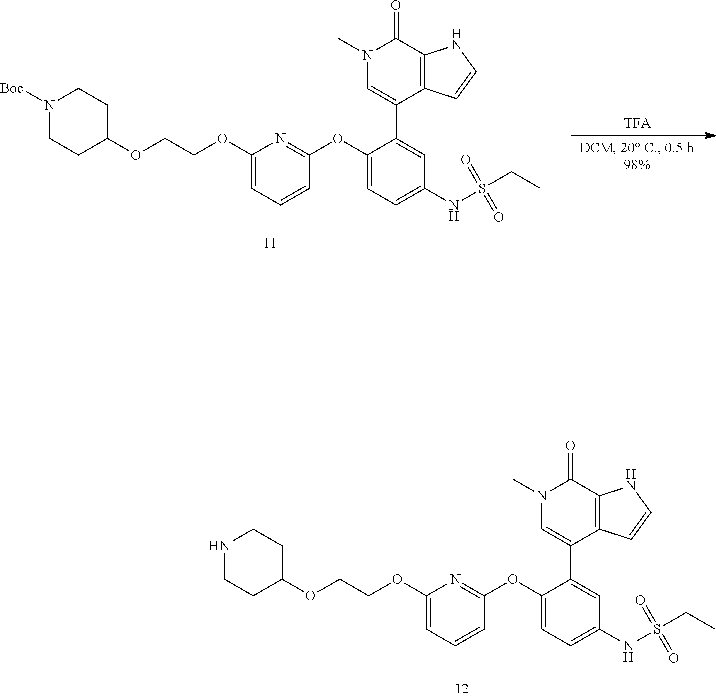 [SMILES: CCS(=O)(=O)Nc1ccc(Oc2cccc(OCCOC3CCN(C(=O)OC(C)(C)C)CC3)n2)c(-c2cn(C)c(=O)c3[nH]ccc23)c1 ; CCS(=O)(=O)Nc1ccc(Oc2cccc(OCCOC3CCNCC3)n2)c(-c2cn(C)c(=O)c3[nH]ccc23)c1]

To a solution of tert-butyl 4-[2-[[6-[4-(ethylsulfonylamino)-2-(6-methyl-7-oxo-1H-pyrrolo[2,3-c]pyridin-4-yl)phenoxy]-2-pyridyl]oxy]ethoxy]piperidine-1-carboxylate (50 mg, 75 mol, 1.0 equiv) in DCM (5 mL) was added TFA (500 μL), the mixture was stirred at 20° C. for 0.5 h and concentrated to give N-[3-(6-methyl-7-oxo-1H-pyrrolo[2,3-c]pyridin-4-yl)-4-[[6-[2-(4-piperidyloxy)ethoxy]-2-pyridyl]oxy]phenyl]ethanesulfonamide (50 mg, TFA salt) as a yellow gum.

### 2.0 Synthesis of Compound I-47

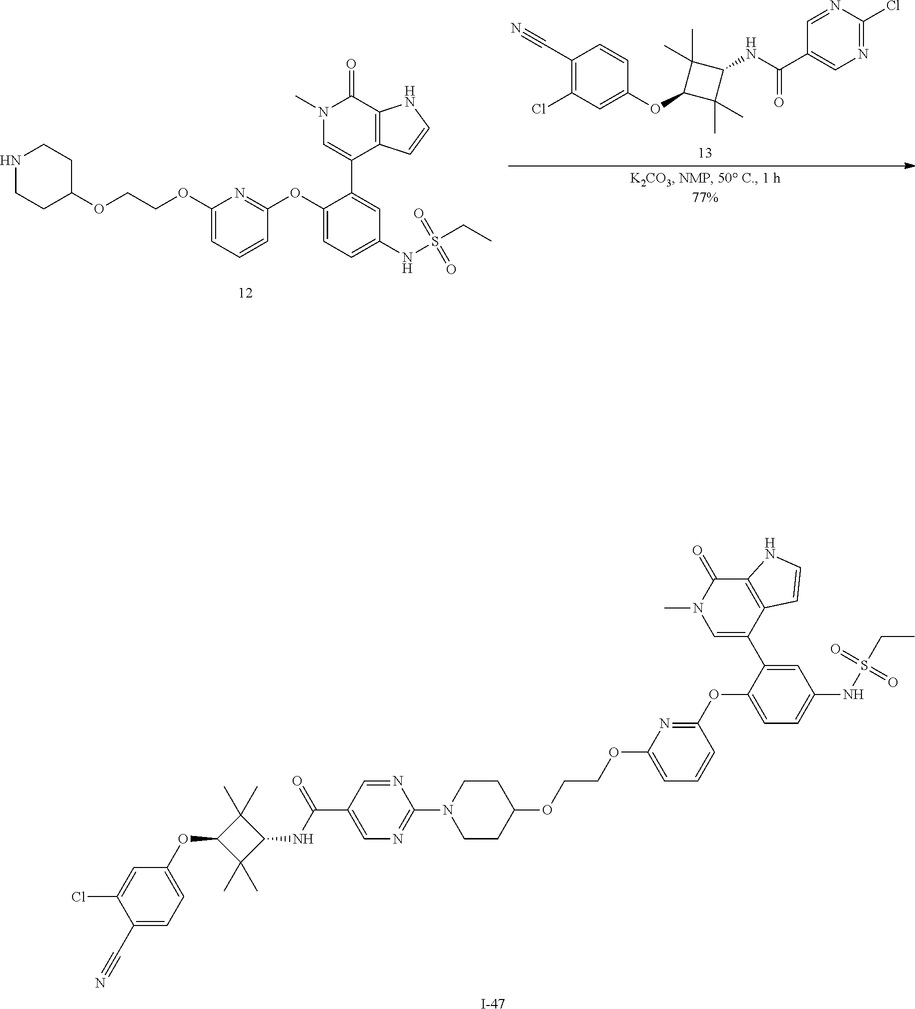

To a solution of N-[3-(6-methyl-7-oxo-1H-pyrrolo[2,3-c]pyridin-4-yl)-4-[[6-[2-(4-piperidyloxy)ethoxy]-2-pyridyl]oxy]phenyl]ethanesulfonamide (50 mg, 73 mol, 1.0 equiv, TFA salt) in NMP (3 mL) was added K₂CO₃ (30 mg, 220 μmol, 3.0 equiv) and tert-butyl N-[3-[5-(2,5-difluorophenyl)-3-[methyl-[2-[2-[2-[2-[2-(4-piperidyloxy)ethoxy]ethoxy]ethoxy]ethoxy]ethyl]carbamoyl]-2-phenyl-1,3,4-thiadiazol-2-yl]propyl]carbamate (30 mg, 73 mol, 1.0 equiv). The mixture was stirred at 50° C. for 1 h. The residue was purified by prep-HPLC (column: Unisil 3-100 C18 Ultra 150 mm*50 mm*3 μm; mobile phase: [water(FA)-ACN]; B %: 45%-75%, 7 min) to give N-[3-(3-chloro-4-cyano-phenoxy)-2,2,4,4-tetramethyl-cyclobutyl]-2-[4-[2-[[6-[4-(ethylsulfonylamino)-2-(6-methyl-7-oxo-1Hpyrrolo[2,3-c]pyridin-4-yl)phenoxy]-2-pyridyl]oxy]ethoxy]-1-piperidyl]pyrimidine-5-carboxamide (55 mg, 77% yield) as a white solid. ¹H NMR (400 MHz, DMSO-d₆): δ 12.05 (s, 1H), 9.86 (s, 1H), 8.78 (s, 2H), 7.91 (d, J=8.8 Hz, 1H), 7.78-7.76 (m, 1H), 7.62-7.58 (m, 1H), 7.45 (s, 1H), 7.15-7.10 (m, 5H), 7.00-6.98 (m, 1H), 6.38-6.30 (m, 2H), 6.20 (s, 1H), 4.30 (s, 1H), 4.24-4.20 (m, 2H), 4.05-4.03 (m, 3H), 3.62-3.50 (m, 3H), 3.47-3.42 (m, 5H), 3.15-3.13 (m, 2H), 1.84-1.81 (m, 2H), 1.38-1.36 (m, 2H), 1.26-1.24 (m, 2H), 1.23-1.21 (m, 9H), 1.12 (s, 9H).

### Example 5—Synthesis of N-((1r, 3r)-3-(3-chloro-4-cyanophenoxy)-2,2,4,4-tetramethylcyclobutyl)-2-(4-(2-(3-(4-(cyclopropanesulfonamido)-2-(6-methyl-7-oxo-6,7-dihydro-1H-pyrrolo[2,3-c]pyridin-4-yl)phenoxy)phenoxy)ethoxy)piperidin-1-yl)pyrimidine-5-carboxamide (I-48)

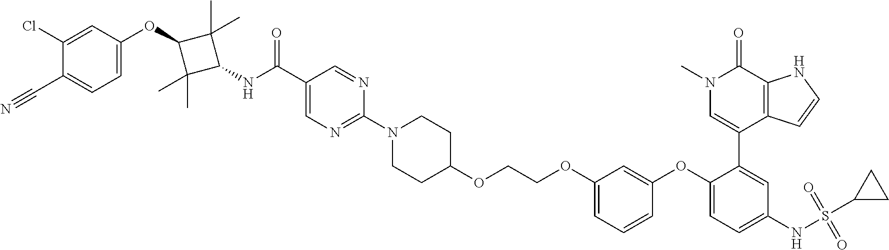 [SMILES: Cn1cc(-c2cc(NS(=O)(=O)C3CC3)ccc2Oc2cccc(OCCOC3CCN(c4ncc(C(=O)N[C@H]5C(C)(C)[C@H](Oc6ccc(C#N)c(Cl)c6)C5(C)C)cn4)CC3)c2)c2cc[nH]c2c1=O]

### 1.1 Synthesis of Compound 2

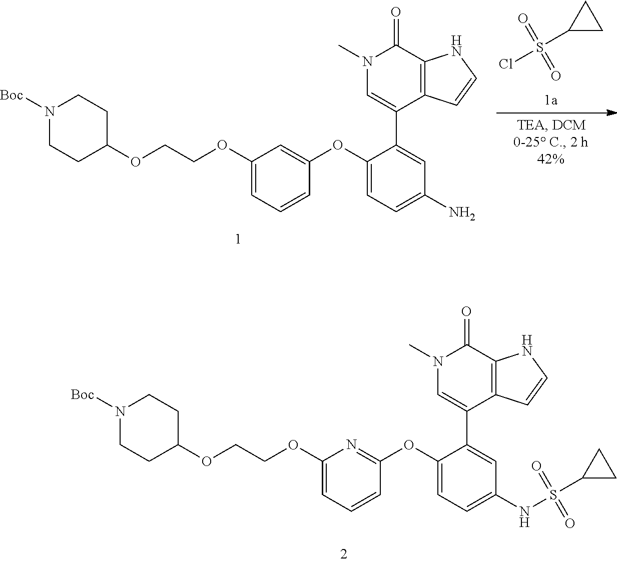

To a solution of tert-butyl 4-[2-[3-[4-amino-2-(6-methyl-7-oxo-1H-pyrrolo[2,3-c]pyridin-4-yl)phenoxy]phenoxy]ethoxy]piperidine-1-carboxylate (50 mg, 87 mol, 1.0 equiv) in DCM (1 mL) was added TEA (106 mg, 1 gmol, 12 equiv) and cyclopropanesulfonyl chloride (245 mg, 2 mol, 20 equiv) at 0° C. The mixture was stirred at 25° C. for 2 h and concentrated. The residue was purified by prep-TLC (SiO₂, DCM: MeOH=10/1) to give tert-butyl 4-[2-[3-[4-(cyclopropylsulfonylamino)-2-(6-methyl-7-oxo-1H-pyrrolo[2,3-c]pyridin-4-yl)phenoxy]phenoxy]ethoxy]piperidine-1-carboxylate (25 mg, 42% yield). ¹H NMR (400 MHz, CDCl₃): δ 9.84 (s, 1H), 7.51 (d, J=2.8 Hz, 1H), 7.23-7.26 (m, 2H), 7.06-7.08 (m, 2H), 7.03 (d, J=6.4 Hz, 2H), 6.57 (m, 1H), 6.47 (m, 1H), 6.43-6.44 (m, 2H), 4.01 (t, J=4.8 Hz, 2H), 3.75 (s, 2H), 3.50-3.56 (m, 2H), 3.04-3.10 (m, 2H), 2.53-2.59 (m, 1H), 1.80-1.83 (m, 1H), 1.63 (s, 3H), 1.48-1.56 (m, 4H), 1.46 (s, 9H), 1.21-1.24 (m, 2H), 1.02 (m, 2H).

### 1.2 Synthesis of Compound 3

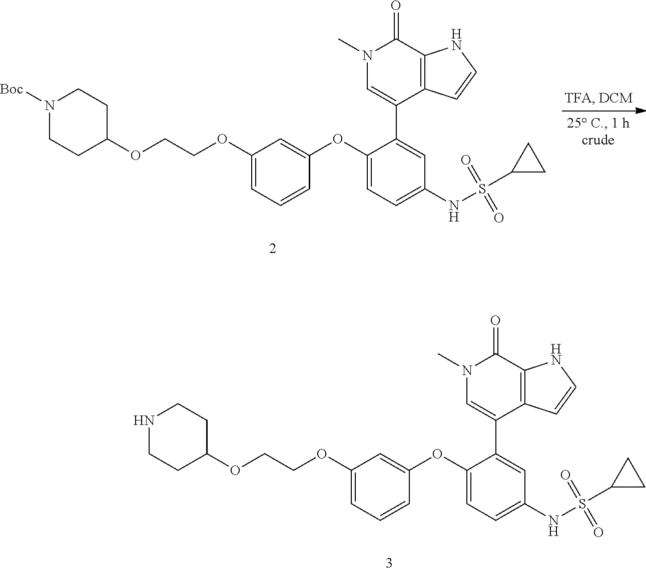 [SMILES: Cn1cc(-c2cc(NS(=O)(=O)C3CC3)ccc2Oc2cccc(OCCOC3CCN(C(=O)OC(C)(C)C)CC3)c2)c2cc[nH]c2c1=O ; Cn1cc(-c2cc(NS(=O)(=O)C3CC3)ccc2Oc2cccc(OCCOC3CCNCC3)c2)c2cc[nH]c2c1=O]

To a solution of tert-butyl 4-[2-[3-[4-(cyclopropylsulfonylamino)-2-(6-methyl-7-oxo-1H-pyrrolo[2,3-c]pyridin-4-yl)phenoxy]phenoxy]ethoxy]piperidine-1-carboxylate (25 mg, 37 μmol, 1.0 equiv) in DCM (1 mL) was added TFA (0.2 mL). The mixture was stirred at 25° C. for 1 h and concentrated under reduced pressure to give a crude product.

### 1.3 Synthesis of I-48

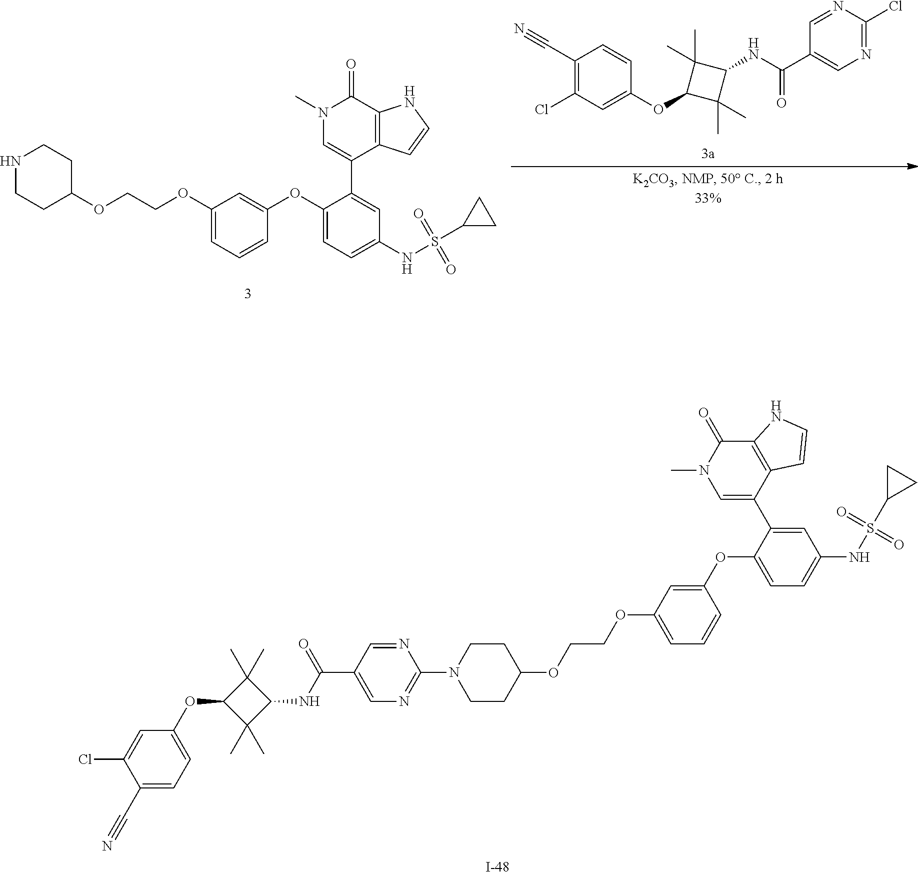

To a solution of N-[3-(6-methyl-7-oxo-1H-pyrrolo[2,3-c]pyridin-4-yl)-4-[3-[2-(4-piperidyloxy)ethoxy]phenoxy]phenyl]cyclopropanesulfonamide (25 mg, 37 mol, 1.0 equiv, TFA) in NMP (1 mL) was added K₂CO₃ (15 mg, 110 mol, 3.0 equiv) and 2-chloro-N-[3-(3-chloro-4-cyano-phenoxy)-2,2,4,4-tetramethyl-cyclobutyl]pyrimidine-5-carboxamide (15 mg, 37 mol, 1.0 equiv). The mixture was stirred at 50° C. for 2 h. The reaction mixture was filtered and concentrated. The residue was purified by prep-HPLC (column: Waters Xbridge 150 mm*25 mm*5 μm; mobile phase: [water(NH₄HCO₃)-ACN]; B %: 52%-82%, 9 min) to give N-[3-(3-chloro-4-cyano-phenoxy)-2,2,4,4-tetramethyl-cyclobutyl]-2-[4-[2-[3-[4-(cyclopropyl-sulfonylamino)-2-(6-methyl-7-oxo-1H-pyrrolo[2,3-c]pyridin-4-yl)phenoxy]phenoxy]ethoxy]-1-piperidyl]pyrimidine-5-carboxamide (12 mg, 33% yield) as a white solid. ¹H NMR (400 MHz, DMSO-d₆): δ 12.05 (s, 1H), 9.72 (s, 1H), 8.75 (s, 2H), 7.90 (d, J=8.8 Hz, 1H), 7.72 (d, J=9.0 Hz, 1H), 7.42 (d, J=2.6 Hz, 1H), 7.27-7.29 (m, 2H), 7.21-7.24 (m, 2H), 7.12-7.16 (m, 1H), 7.06 (d, J=8.8 Hz, 1H), 7.01 (m, 1H), 6.58 (m, 1H), 6.39-6.41 (m, 2H), 6.27 (d, J=2.4 Hz, 1H), 4.20-4.29 (m, 4H), 4.03 (d, J=9.2 Hz, 1H), 3.97-4.00 (m, 2H), 3.71-3.73 (m, 2H), 3.65 (m, 2H), 3.47 (s, 1H), 3.46 (s, 1H), 3.43 (d, J=3.6 Hz, 1H), 3.31 (s, 3H), 1.85-1.89 (m, 2H), 1.37 (s, 4H), 1.21 (s, 6H), 1.11 (s, 6H).

### Example 6—Synthesis of N-((1r, 3r)-3-(3-chloro-4-cyanophenoxy)-2,2,4,4-tetramethylcyclobutyl)-2-(4-(2-(3-(2-(6-methyl-7-oxo-6,7-dihydro-1H-pyrrolo[2,3-c]pyridin-4-yl)-4-(methylsulfonamido)phenoxy)phenoxy)ethoxy)piperidin-1-yl)pyrimidine-5-carboxamide (I-50)

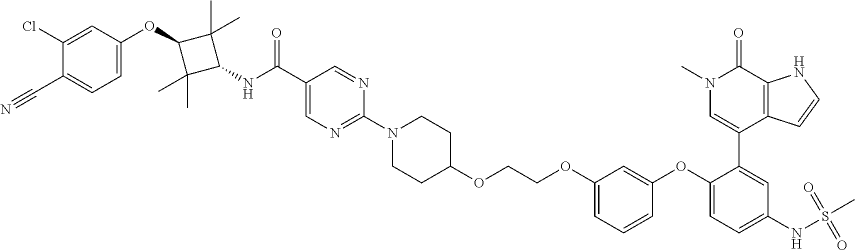 [SMILES: Cn1cc(-c2cc(NS(C)(=O)=O)ccc2Oc2cccc(OCCOC3CCN(c4ncc(C(=O)N[C@H]5C(C)(C)[C@H](Oc6ccc(C#N)c(Cl)c6)C5(C)C)cn4)CC3)c2)c2cc[nH]c2c1=O]

### 1.1 Synthesis of Compound 2

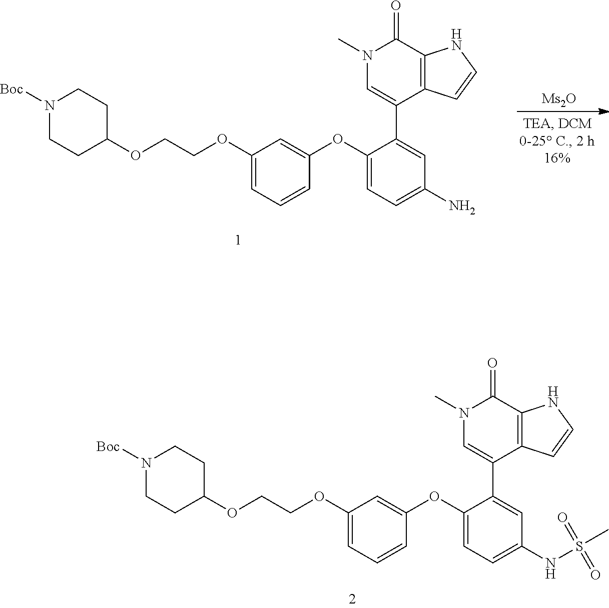 [SMILES: "Ms2O" ; Cn1cc(-c2cc(N)ccc2Oc2cccc(OCCOC3CCN(C(=O)OC(C)(C)C)CC3)c2)c2cc[nH]c2c1=O ; Cn1cc(-c2cc(NS(C)(=O)=O)ccc2Oc2cccc(OCCOC3CCN(C(=O)OC(C)(C)C)CC3)c2)c2cc[nH]c2c1=O]

To a solution of tert-butyl 4-[2-[3-[4-amino-2-(6-methyl-7-oxo-1H-pyrrolo[2,3-c]pyridin-4-yl)phenoxy]phenoxy]ethoxy]piperidine-1-carboxylate (50 mg, 87 mol, 1.0 equiv) in DCM (1 mL) was added TEA (26 mg, 261 μmol, 3.0 equiv) and methylsulfonyl methanesulfonate (30 mg, 174 mol, 2.0 equiv) at 0° C. The mixture was stirred at 25° C. for 2 h and concentrated. The residue was purified by prep-TLC (SiO₂, DCM: MeOH=10/1) to give tert-butyl 4-[2-[3-[4-(methanesulfonamido)-2-(6-methyl-7-oxo-1H-pyrrolo[2,3-c]pyridin-4-yl)phenoxy]phenoxy]ethoxy]piperidine-1-carboxylate (10 mg, 16% yield).

### 1.2 Synthesis of Compound 3

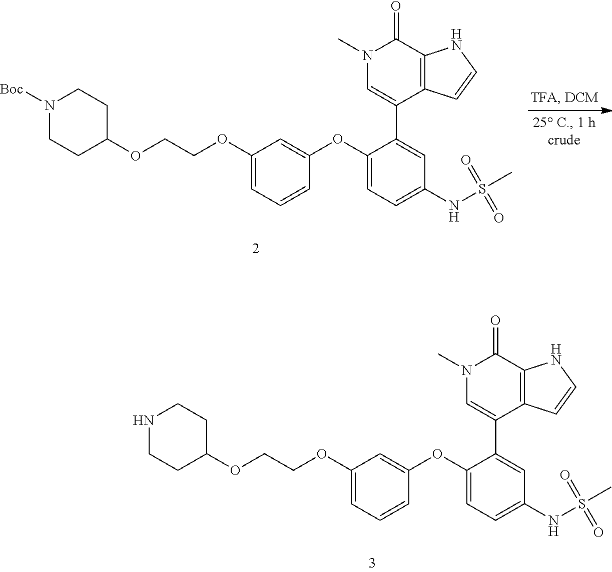 [SMILES: Cn1cc(-c2cc(NS(C)(=O)=O)ccc2Oc2cccc(OCCOC3CCN(C(=O)OC(C)(C)C)CC3)c2)c2cc[nH]c2c1=O ; Cn1cc(-c2cc(NS(C)(=O)=O)ccc2Oc2cccc(OCCOC3CCNCC3)c2)c2cc[nH]c2c1=O]

To a solution of tert-butyl 4-[2-[3-[4-(methanesulfonamido)-2-(6-methyl-7-oxo-1H-pyrrolo[2,3-c]pyridin-4-yl)phenoxy]phenoxy]ethoxy]piperidine-1-carboxylate (10 mg, 15 μmol, 1.0 equiv) in DCM (1 mL) was added TFA (0.2 mL). The mixture was stirred at 25° C. for 1 h and concentrated under reduced pressure to give a crude product.

### 1.3 Synthesis of I-50

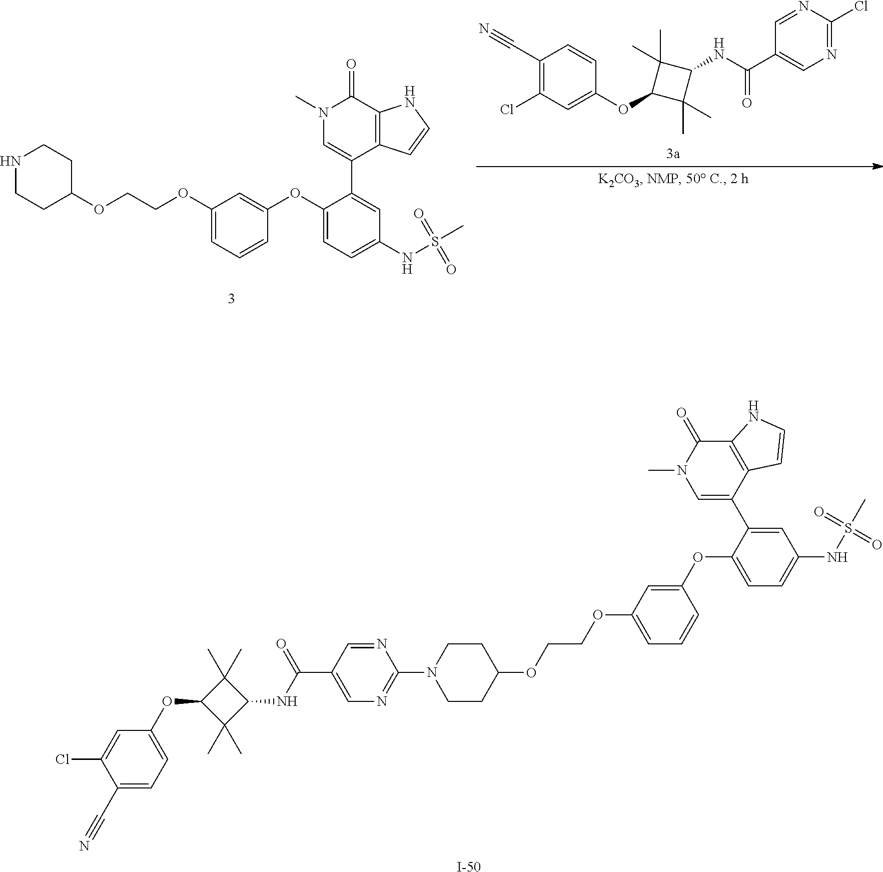

To a solution of N-[3-(6-methyl-7-oxo-1H-pyrrolo[2,3-c]pyridin-4-yl)-4-[3-[2-(4-pipendyloxy)ethoxy]phenoxy]phenyl]methanesulfonamide (10 mg, 15 mol, 1.0 equiv, TFA) in NMP (1 mL) was added K₂CO₃ (δ mg, 45 μmol, 3.0 equiv) and 2-chloro-N-[3-(3-chloro-4-cyano-phenoxy)-2,2,4,4-tetramethyl-cyclobutyl]pyrimidine-5-carboxamide (δ mg, 15 μmol, 1.0 equiv). The mixture was stirred at 50° C. for 2 h. The reaction mixture was filtered and concentrated. The residue was purified by prep-HPLC (column: Waters Xbridge 150 mm*25 mm*5 μm; mobile phase: [water(NH₄HCO₃)-ACN]; B %: 50%-80%, 9 min) to give N-[3-(3-chloro-4-cyano-phenoxy)-2,2,4,4-tetramethyl-cyclobutyl]-2-[4-[2-[3-[4-(methanesulfonamido)-2-(6-methyl-7-oxo-1H-pyrrolo[2,3-c]pyridin-4-yl)phenoxy]phenoxy]ethoxy]-1-piperidyl]pyrimidine-5-carboxamide (3 mg, 24% yield) as a white solid. ¹H NMR (400 MHz, CDCl₃): δ 9.99 (s, 1H), 8.74 (s, 2H), 7.57 (d, J=8.6 Hz, 1H), 7.48 (d, J=2.6 Hz, 1H), 7.38 (s, 1H), 7.21-7.26 (m, 2H), 7.14 (t, J=8.2 Hz, 1H), 7.04-7.10 (m, 2H), 6.97 (d, J=2.4 Hz, 1H), 6.81 (m, 1H), 6.58 (m, 1H), 6.49 (m, 1H), 6.43 (m, 2H), 6.11 (d, J=8.6 Hz, 1H), 4.18-4.24 (m, 2H), 4.15 (d, J=8.0 Hz, 1H), 4.04-4.06 (m, 2H), 3.80 (t, J=4.6 Hz, 2H), 3.65-3.70 (m, 1H), 3.62 (d, J=3.2 Hz, 1H), 3.59 (s, 3H), 3.08 (s, 3H), 1.85-1.94 (m, 2H), 1.59-1.66 (m, 4H), 1.24 (d, J=17.6 Hz, 12H).

### Example 7—Synthesis of N-((1r, 3r)-3-(3-chloro-4-cyanophenoxy)-2,2,4,4-tetramethylcyclobutyl)-2-(4-(2-(3-(2-(6-methyl-7-oxo-6,7-dihydro-1H-pyrrolo[2,3-c]pyridin-4-yl)-4-((methylsulfonyl)methyl)phenoxy)phenoxy)ethoxy)piperidin-1-yl)pyrimidine-5-carboxamide (I-53)

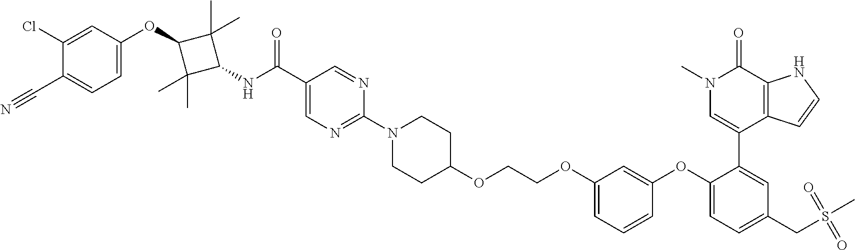 [SMILES: Cn1cc(-c2cc(CS(C)(=O)=O)ccc2Oc2cccc(OCCOC3CCN(c4ncc(C(=O)N[C@H]5C(C)(C)[C@H](Oc6ccc(C#N)c(Cl)c6)C5(C)C)cn4)CC3)c2)c2cc[nH]c2c1=O]

### 1.1 Synthesis of Compound 2

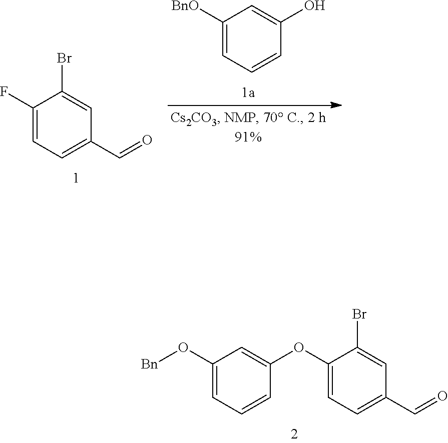 [SMILES: O=Cc1ccc(F)c(Br)c1 ; Oc1cccc(OCc2ccccc2)c1 ; O=Cc1ccc(Oc2cccc(OCc3ccccc3)c2)c(Br)c1]

To a solution of 3-benzyloxyphenol (5 g, 25 mol, 1 equiv) in NMP (30 mL) was added Cs₂CO₃ (24.4 g, 75 mol, 3 equiv) and 3-bromo-4-fluoro-benzaldehyde (5.1 g, 25 μmol, 1 equiv). The mixture was stirred at 70° C. for 2 h and concentrated. The residue was purified by column chromatography (SiO₂, Petroleum ether/Ethyl acetate=20/1 to 5/1) to give 4-(3-benzyloxyphenoxy)-3-bromo-benzaldehyde (8.72 g, 91% yield). ¹H NMR (400 MHz, DMSO-d₆) δ 5.12 (s, 2H), 6.68-6.69 (m, 1H), 6.81˜6.82 (m, 1H), 6.91˜6.93 (m, 1H), 7.05 (d, J=8.36 Hz, 1H), 7.33-7.46 (m, 6H), 7.87˜7.88 (m, 1H), 8.26 (d, J=2.00 Hz, 1H), 9.93 (s, 1H).

### 1.2 Synthesis of Compound 3

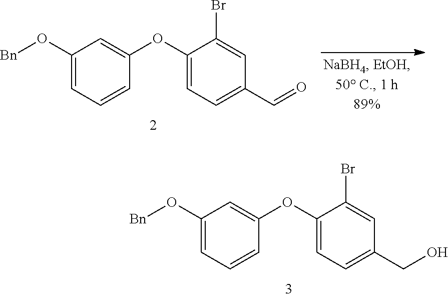 [SMILES: O=Cc1ccc(Oc2cccc(OCc3ccccc3)c2)c(Br)c1 ; OCc1ccc(Oc2cccc(OCc3ccccc3)c2)c(Br)c1]

To a solution of 4-(3-benzyloxyphenoxy)-3-bromo-benzaldehyde (8.72 g, 23 μmol, 1 equiv) in EtOH (80 mL) was added NaBH₄ (1.29 g, 34.1 mol, 1.5 equiv). The mixture was stirred at 50° C. for 1 h. The residue was purified by column chromatography (SiO₂, Petroleum ether/Ethyl acetate=10/1 to 5/1) to give [4-(3-benzyloxyphenoxy)-3-bromo-phenyl]methanol (7.8 g, 89% yield) as a yellow oil. ¹H NMR (400 MHz, CDCl₃) δ 1.65 (s, 1H), 4.60 (s, 2H), 4.95 (s, 2H), 6.43-6.58 (m, 2H), 6.65-6.66 (m, 1H), 6.90 (d, J=8.24 Hz, 1H), 7.08-7.35 (m, 7H), 7.55-7.64 (m, 1H).

### 1.3 Synthesis of Compound 4

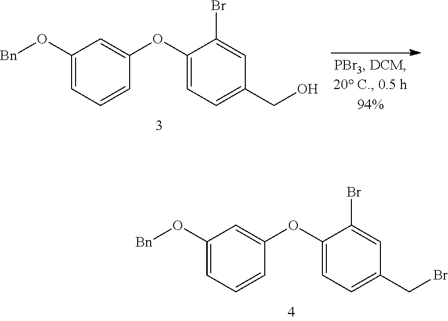 [SMILES: OCc1ccc(Oc2cccc(OCc3ccccc3)c2)c(Br)c1 ; BrCc1ccc(Oc2cccc(OCc3ccccc3)c2)c(Br)c1]

To a solution of [4-(3-benzyloxyphenoxy)-3-bromo-phenyl]methanol (7.8 g, 20 μmol, 1 equiv) in DCM (30 mL) was added PBr₃ (8.22 g, 30 mol, 1.5 equiv) at 0° C. The mixture was stirred at 20° C. for 0.5 h and concentrated. The residue was purified by column chromatography (SiO₂, Petroleum ether/Ethyl acetate=20/1 to 8/1) to give 1-(3-benzyloxyphenoxy)-2-bromo-4-(bromomethyl)benzene (8.52 g, 94% yield). ¹H NMR (400 MHz, CDCl₃) δ 4.37 (s, 2H), 4.95 (s, 2H), 6.46-6.56 (m, 2H), 6.65-6.71 (m, 1H), 6.82 (d, J=8.36 Hz, 1H), 7.16-7.20 (m, 2H), 7.24-7.34 (m, 5H), 7.58 (d, J=2.24 Hz, 1H).

### 1.4 Synthesis of Compound 5

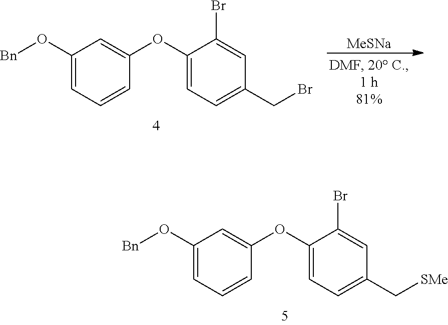 [SMILES: "MeSNa" ; BrCc1ccc(Oc2cccc(OCc3ccccc3)c2)c(Br)c1 ; CSCc1ccc(Oc2cccc(OCc3ccccc3)c2)c(Br)c1]

To a solution of 1-(3-benzyloxyphenoxy)-2-bromo-4-(bromomethyl)benzene (2 g, 4.46 mol, 1 equiv) in DMF (10 mL) was added sodium;methanethiolate (460 mg, 6.56 mol, 1.47 equiv). The mixture was stirred at 20° C. for 1 h. The mixture was filtered and concentrated. The residue was purified by column chromatography (SiO₂, Petroleum ether/Ethyl acetate=20/1 to 8/1) to give 1-(3-benzyloxyphenoxy)-2-bromo-4-(methylsulfanyl-methyl)benzene (1.51 g, 810% yield) as a yellow oil. ¹H NMR (400 MHz, CDCl₃) δ 2.17 (s, 3H), 3.77 (s, 2H), 5.17 (s, 2H), 6.60-6.80 (m, 2H), 6.86˜6.87 (m, 1H), 7.06 (d, J=8.32 Hz, 1H), 7.28-7.39 (m, 2H), 7.44-7.58 (m, 5H), 7.71 (d, J=1.96 Hz, 1H).

### 1.5 Synthesis of Compound 6

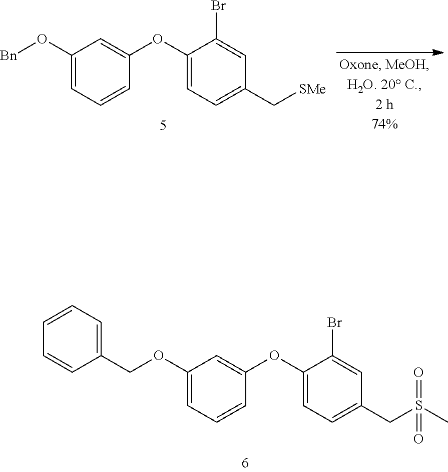 [SMILES: CSCc1ccc(Oc2cccc(OCc3ccccc3)c2)c(Br)c1 ; CS(=O)(=O)Cc1ccc(Oc2cccc(OCc3ccccc3)c2)c(Br)c1]

To a solution of 1-(3-benzyloxyphenoxy)-2-bromo-4-(methylsulfanylmethyl) benzene (1.51 g, 3.64 mol, 1 equiv) in MeOH (20 mL) was added Oxone (4.69 g, 7.63 mol, 2.1 equiv) in H₂O (20 mL). The mixture was stirred at 20° C. for 2 h. The residue was purified by column chromatography (SiO₂, Petroleum ether/Ethyl acetate=20/1 to 1/1) to give 1-(3-benzyloxyphenoxy)-2-bromo-4-(methylsulfonylmethyl) benzene (1.2 g, 74% yield) as a yellow oil. ¹H NMR (400 MHz, CDCl₃) δ 2.85 (s, 3H), 4.19-4.28 (m, 2H), 5.07 (s, 2H), 6.55-6.71 (m, 2H), 6.81˜6.82 (m, 1H), 6.97 (d, J=8.36 Hz, 1H), 7.28-7.37 (m, 3H), 7.38-7.48 (m, 4H), 7.70 (d, J=2.12 Hz, 1H).

### 1.6 Synthesis of Compound 7

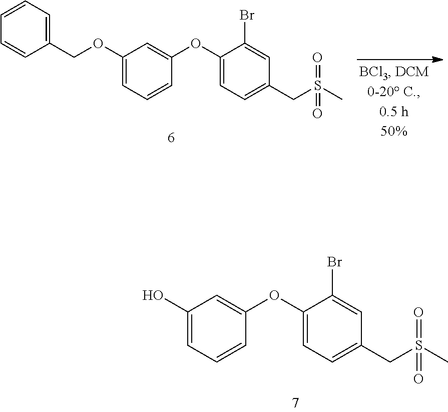 [SMILES: CS(=O)(=O)Cc1ccc(Oc2cccc(OCc3ccccc3)c2)c(Br)c1 ; CS(=O)(=O)Cc1ccc(Oc2cccc(O)c2)c(Br)c1]

To a solution of 1-(3-benzyloxyphenoxy)-2-bromo-4-(methylsulfonylmethyl) benzene (1.2 g, 2.7 μmol, 1 equiv) in DCM (30 mL) was added BCl₃ (1 M, 15 mL, 5.6 equiv) at 0° C. The mixture was stirred at 20° C. for 0.5 h. The residue was purified by column chromatography (SiO₂, Petroleum ether/Ethyl acetate=10/1 to 1/1) to give 3-[2-bromo-4-(methylsulfonylmethyl)phenoxy]phenol (480 mg, 50% yield) as a white solid. ¹H NMR (400 MHz, DMSO-d₆) δ 2.94-2.95 (m, 3H), 4.52 (s, 2H), 6.27-6.34 (m, 1H), 6.38-6.42 (m, 1H), 6.56 (m, 1H), 7.11 (d, J=8.36 Hz, 1H), 7.14-7.18 (m, 1H), 7.43 (m, 1H), 7.75-7.78 (m, 1H), 9.58-9.72 (m, 1H).

### 1.7 Synthesis of Compound 8

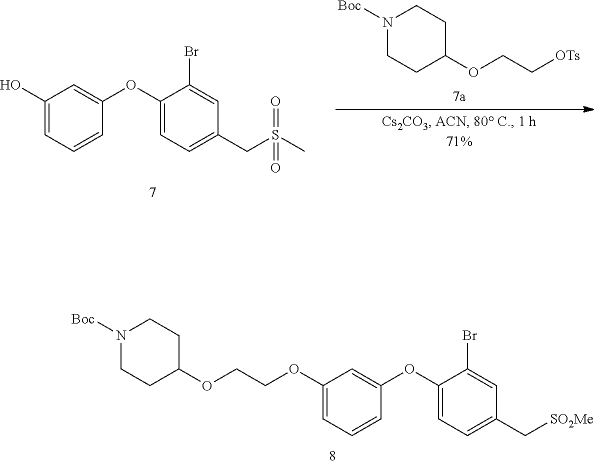 [SMILES: CS(=O)(=O)Cc1ccc(Oc2cccc(O)c2)c(Br)c1 ; Cc1ccc(S(=O)(=O)OCCOC2CCN(C(=O)OC(C)(C)C)CC2)cc1 ; CC(C)(C)OC(=O)N1CCC(OCCOc2cccc(Oc3ccc(CS(C)(=O)=O)cc3Br)c2)CC1]

To a solution of tert-butyl 4-[2-(p-tolylsulfonyloxy)ethoxy]piperidine-1-carboxylate (336 mg, 840 μmol, 1 equiv) in ACN (8 mL) was added Cs₂CO₃ (547 mg, 1.68 μmol, 2 equiv) and 3-[2-bromo-4-(methylsulfonylmethyl)phenoxy]phenol (300 mg, 840 mol, 1 equiv). The mixture was stirred at 80° C. for 1 h. The residue was purified by prep-HPLC (column: Waters Xbridge C18 150 mm*50 mm*10μm; mobile phase: [water(NH₄HCO₃)-ACN]; B %: 44%-74%, 10 min) to give tert-butyl 4-[2-[3-[2-bromo-4-(methylsulfonylmethyl)phenoxy]phenoxy]ethoxy]piperidine-1-carboxylate (350 mg, 71% yield) as a yellow oil.

### 1.8 Synthesis of 9

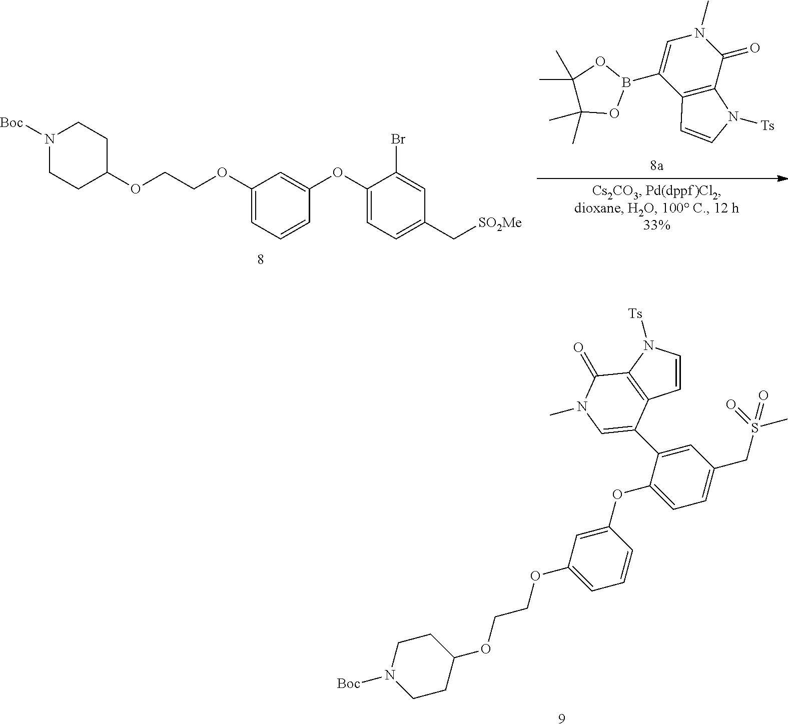 [SMILES: CC(C)(C)OC(=O)N1CCC(OCCOc2cccc(Oc3ccc(CS(C)(=O)=O)cc3Br)c2)CC1 ; Cc1ccc(S(=O)(=O)n2ccc3c(B4OC(C)(C)C(C)(C)O4)cn(C)c(=O)c32)cc1 ; Cc1ccc(S(=O)(=O)n2ccc3c(-c4cc(CS(C)(=O)=O)ccc4Oc4cccc(OCCOC5CCN(C(=O)OC(C)(C)C)CC5)c4)cn(C)c(=O)c32)cc1]

To a solution of tert-butyl 4-[2-[3-[2-bromo-4-(methylsulfonylmethyl)phenoxy]phenoxy]ethoxy]piperidine-1-carboxylate (350 mg, 599 mol, 1 equiv) in dioxane (8 mL) and H₂O (1.6 mL) was added Cs₂CO₃ (585 mg, 1.80 μmol, 3 equiv) and 6-methyl-1-(p-tolylsulfonyl)-4-(4,4,5,5-tetramethyl-1,3,2-dioxaborolan-2-yl)pyrrolo[2,3-c]pyridin-7-one (308 mg, 719 μmol, 1.2 equiv), Pd(dppf)Cl₂ (88 mg, 120 gmol, 0.2 equiv. The mixture was stirred at 100° C. for 12 h and concentrated. The residue was purified by prep-HPLC (column: Waters Xbridge C18 150 mm*50 mm*10 μm; mobile phase: [water(NH₄HCO₃)-ACN]; B %: 40%-40%, 10 min) to give tert-butyl 4-[2-[3-[2-[6-methyl-7-oxo-1-(p-tolylsulfonyl)pyrrolo [2,3-c]pyridin-4-yl]-4-(methylsulfonylmethyl)phenoxy]phenoxy]ethoxy]piperidine-1-carboxylate (160 mg, 33% yield). ¹H NMR (400 MHz, CDCl₃) δ 1.45-1.54 (m, 11H), 1.82-1.89 (m, 2H), 2.41 (s, 3H), 2.88 (s, 3H), 3.07˜3.08 (m, 2H), 3.50-3.57 (m, 4H), 3.74-3.83 (m, 4H), 4.03˜4.04 (m, 2H), 4.26 (s, 2H), 6.45-6.51 (m, 3H), 6.63˜6.64 (m, 1H), 7.03 (d, J=8.36 Hz, 1H), 7.12-7.19 (m, 2H), 7.30-7.34 (m, 3H), 7.47 (d, J=2.12 Hz, 1H), 7.90 (d, J=3.52 Hz, 1H), 8.02 (d, J=8.36 Hz, 2H).

### 1.9 Synthesis of 10

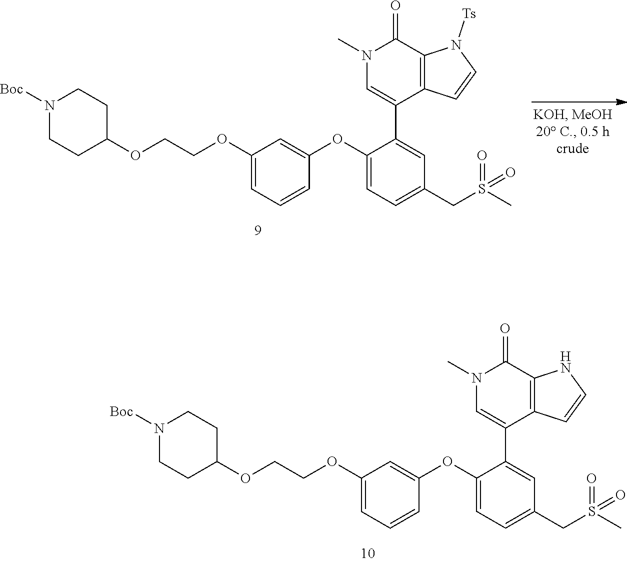 [SMILES: Cc1ccc(S(=O)(=O)n2ccc3c(-c4cc(CS(C)(=O)=O)ccc4Oc4cccc(OCCOC5CCN(C(=O)OC(C)(C)C)CC5)c4)cn(C)c(=O)c32)cc1 ; Cn1cc(-c2cc(CS(C)(=O)=O)ccc2Oc2cccc(OCCOC3CCN(C(=O)OC(C)(C)C)CC3)c2)c2cc[nH]c2c1=O]

To a solution of tert-butyl 4-[2-[3-[2-[6-methyl-7-oxo-1-(p-tolylsulfonyl)pyrrolo [2,3-c]pyridin-4-yl]-4-(methylsulfonylmethyl)phenoxy]phenoxy]ethoxy]piperidine-1-carboxylate (160 mg, 199 mol, 1 equiv) in MeOH (4 mL) was added KOH (33 mg, 596 μmol, 3 equiv). The mixture was stirred at 20° C. for 0.5 h. The reaction mixture was filtered and concentrated to give crude product tert-butyl 4-[2-[3-[2-(6-methyl-7-oxo-1H-pyrrolo[2,3-c]pyridin-4-yl)-4-(methylsulfonylmethyl)phenoxy]phenoxy]ethoxy]piperidine-1-carboxylate (128 mg) as a yellow oil.

### 2.0 Synthesis of 11

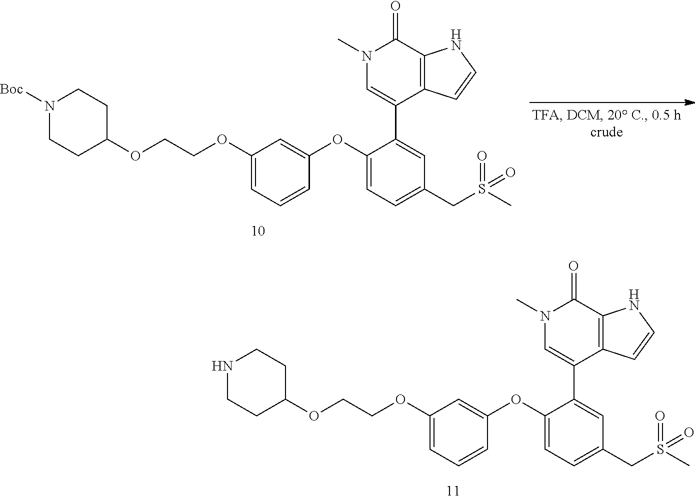 [SMILES: Cn1cc(-c2cc(CS(C)(=O)=O)ccc2Oc2cccc(OCCOC3CCN(C(=O)OC(C)(C)C)CC3)c2)c2cc[nH]c2c1=O ; Cn1cc(-c2cc(CS(C)(=O)=O)ccc2Oc2cccc(OCCOC3CCNCC3)c2)c2cc[nH]c2c1=O]

To a solution of tert-butyl 4-[2-[3-[2-(6-methyl-7-oxo-1H-pyrrolo[2,3-c]pyridin-4-yl)-4-(methylsulfonylmethyl)phenoxy]phenoxy]ethoxy]piperidine-1-carboxylate (128 mg, 196 μmol, 1 equiv) in DCM (4 mL) was added TFA (2 mL). The mixture was stirred at 20° C. for 0.5 h. The mixture was filtered and concentrated to give crude 6-methyl-4-[5-(methylsulfonylmethyl)-2-[3-[2-(4-piperidyloxy)ethoxy]phenoxy]phenyl]-1H-pyrrolo[2,3-c]pyridin 7-one (130 mg, TFA salt) as a yellow oil.

### 2.1 Synthesis of 1-53

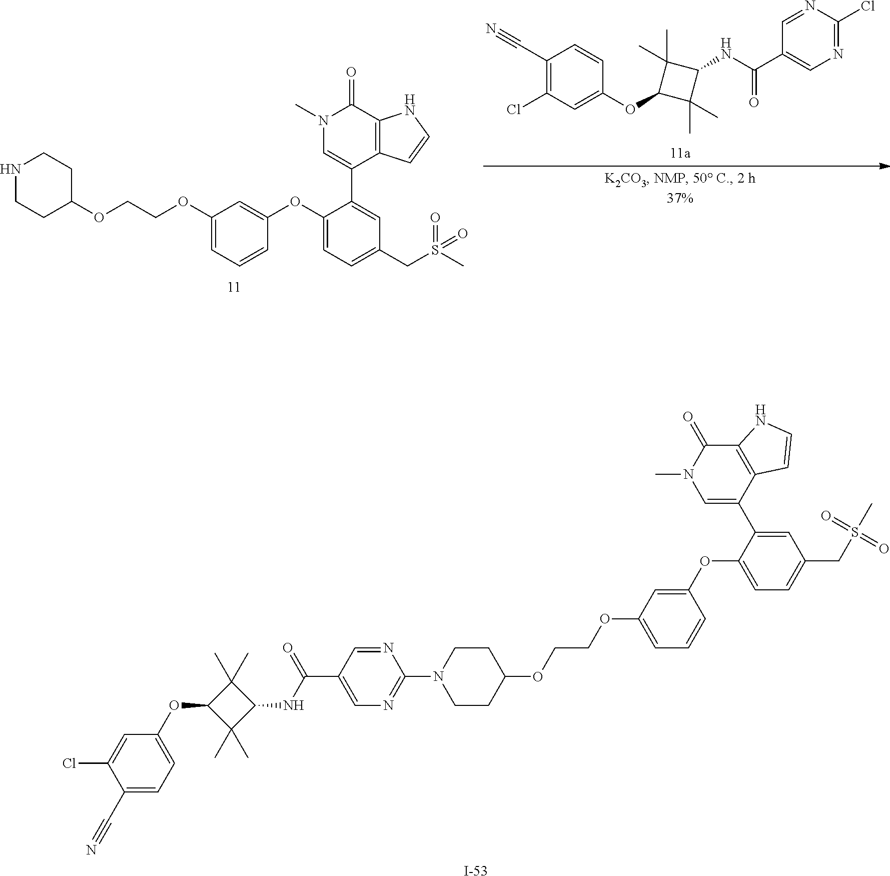

To a solution of 6-methyl-4-[5-(methylsulfonylmethyl)-2-[3-[2-(4-piperidyloxy) ethoxy]phenoxy]phenyl]-1H-pyrrolo[2,3-c]pyridin-7-one (100 mg, 150 μmol, 1 equiv, TFA salt) in NMP (2 mL) was added K₂CO₃ (21 mg, 150 mol, 1 equiv) and 2-chloro-N-[3-(3-chloro-4-cyano-phenoxy)-2,2,4,4-tetramethyl-cyclobutyl]pyrimidine-5-carboxamide (63 mg, 150 μmol, 1 equiv). The mixture was stirred at 50° C. for 2 h. The residue was purified by prep-HPLC (column: Unisil 3-100 C18 Ultra 150 mm*50 mm*3 μm; mobile phase: [water(FA)-ACN]; B %: 46%-76%, 7 min) to give N-[3-(3-chloro-4-cyano-phenoxy)-2,2,4,4-tetramethyl-cyclobutyl]-2-[4-[2-[3-[2-(6-methyl-7-oxo-1H-pyrrolo[2,3-c]pyridin-4-yl)-4-(methylsulfonylmethyl)phenoxy]phenoxy]ethoxy]-1-piperidyl]pyrimidine-5-carboxamide (53 mg, 37% yield). ¹H NMR (400 MHz, MeOD) δ 1.21 (s, 6H), 1.28 (s, 6H), 1.54-1.55 (m, 2H), 1.90-1.91 (m, 2H), 2.96 (s, 3H), 3.55-3.63 (m, 5H), 3.66-3.73 (m, 1H), 3.76-3.81 (m, 2H), 3.98-4.04 (m, 2H), 4.13 (s, 1H), 4.22-4.30 (m, 3H), 4.49 (s, 2H), 6.37-6.47 (m, 3H), 6.58-6.59 (m, 1H), 6.97-6.98 (m, 1H), 7.08-7.15 (m, 3H), 7.24 (s, 1H), 7.32 (d, J=2.88 Hz, 1H), 7.44-7.45 (m, 1H), 7.66 (d, J=2.24 Hz, 1H), 7.72 (d, J=8.76 Hz, 1H), 8.73 (s, 2H).

### Example 8—Synthesis of N-((1r, 3r)-3-(3-chloro-4-cyanophenoxy)-2,2,4,4-tetramethylcyclobutyl)-2-(4-(2-(3-(4-((ethylsulfonyl)methyl)-2-(6-methyl-7-oxo-6,7-dihydro-1H-pyrrolo[2,3-c]pyridin-4-yl)phenoxy)phenoxy)ethoxy)piperidin-1-yl)pyrimidine-5-carboxamide (I-54)

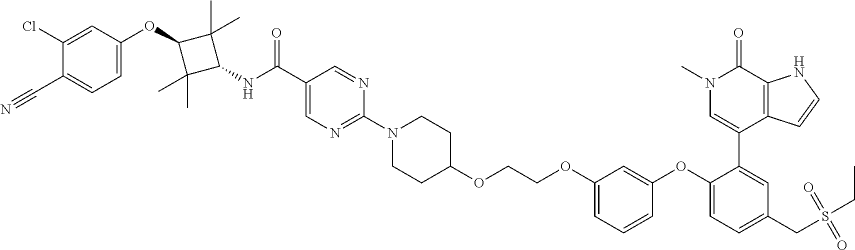 [SMILES: CCS(=O)(=O)Cc1ccc(Oc2cccc(OCCOC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1]

### 1.1 Synthesis of Compound 2

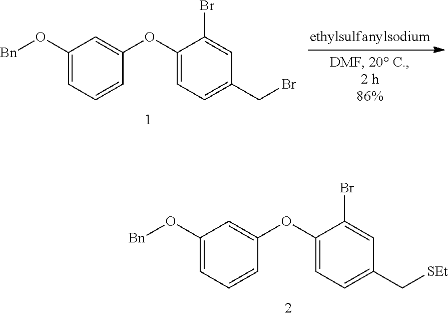

To a solution of 1-(3-benzyloxyphenoxy)-2-bromo-4-(bromomethyl)benzene (1.85 g, 4.13 mol, 1 equiv) in DMF (1 mL) was added ethylsulfanylsodium (521 mg, 6.19 μmol, 1.5 equiv). The mixture was stirred at 20° C. for 2 h and concentrated. The residue was purified by column chromatography (SiO₂, Petroleum ether/Ethyl acetate=10/1 to 5/1) to give 1-(3-benzyloxyphenoxy)-2-bromo-4-(ethylsulfanylmethyl)benzene (1.52 g, 86% yield). ¹H NMR (400 MHz, CDCl₃) δ 1.17˜1.18 (m, 3H), 2.39˜2.40 (m, 2H), 3.60-3.62 (m, 2H), 4.95 (s, 2H), 6.45-6.54 (m, 2H), 6.64˜6.65 (m, 1H), 6.85 (d, J=8.24 Hz, 1H), 7.11-7.17 (m, 2H), 7.24-7.35 (m, 5H), 7.51 (d, 0.1=2.0 Hz, 1H).

### 1.2 Synthesis of Compound 3

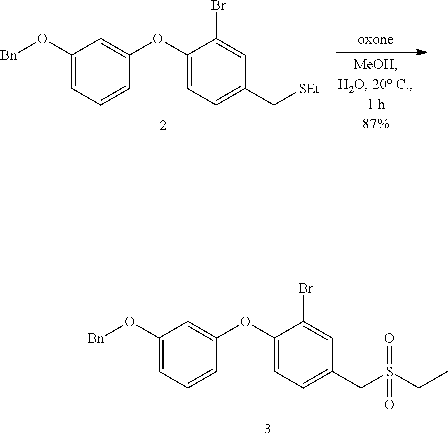

To a solution of 1-(3-benzyloxyphenoxy)-2-bromo-4-(ethylsulfanylmethyl)benzene (1.52 g, 3.54 mol, 1 equiv) in MeOH (20 mL) was added Oxone (4.57 g, 7.43 μmol, 2.1 equiv) in H₂O (20 mL). The mixture was stirred at 20° C. for 1 h. The residue was purified by column chromatography (SiO₂, Petroleum ether/Ethyl acetate=10/1 to 4/1) to give 1-(3-benzyloxyphenoxy)-2-bromo-4-(ethylsulfonylmethyl)benzene (1.42 g, 87% yield) as a white solid.

### 1.3 Synthesis of Compound 4

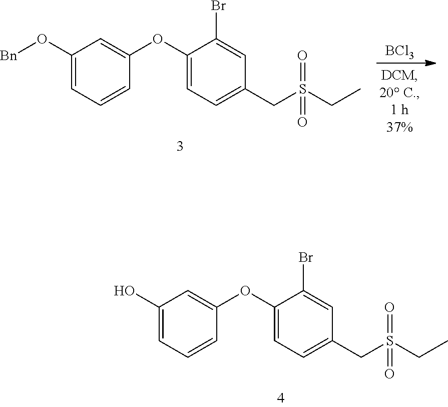 [SMILES: CCS(=O)(=O)Cc1ccc(Oc2cccc(OCc3ccccc3)c2)c(Br)c1 ; "BCl3" ; CCS(=O)(=O)Cc1ccc(Oc2cccc(O)c2)c(Br)c1]

To a solution of 1-(3-benzyloxyphenoxy)-2-bromo-4-(ethylsulfonylmethyl)benzene (1.22 g, 2.64 mol, 1 equiv) in DCM (30 mL) was added BCl₃ (1 M, 26 mL, 10 equiv) at 0° C. The mixture was stirred at 20° C. for 1 h and concentrated. The residue was purified by column chromatography (SiO₂, Petroleum ether/Ethyl acetate=10/1 to 2/1) to give 3-[2-bromo-4-(ethylsulfonylmethyl)phenoxy]phenol (363 mg, 37% yield). ¹H NMR (400 MHz, DMSO-d₆) δ 1.22˜1.26 (m, 3H), 3.04˜3.07 (m, 2H), 4.51 (s, 2H), 6.32 (s, 1H), 6.40˜6.41 (m, 1H), 6.55-6.56 (m, 1H), 7.10 (d, J=8.32 Hz, 1H), 7.17˜7.18 (m, 1H), 7.42˜7.43 (m, 1H), 7.77 (d, J=1.72 Hz, 1H), 9.66 (s, 1H).

### 1.4 Synthesis of Compound 5

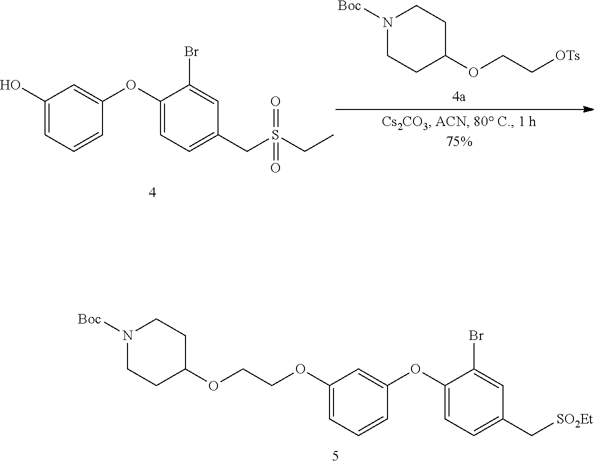

To a solution of 3-[2-bromo-4-(ethylsulfonylmethyl)phenoxy]phenol (363 mg, 978 μmol, 1 equiv) in MeCN (5 mL) was added Cs₂CO₃ (637 mg, 1.96 mol, 2 equiv) and tert-butyl 4-[2-(p-tolylsulfonyloxy)ethoxy]piperidine-1-carboxylate (391 mg, 978 μmol, 1 equiv). The mixture was stirred at 80° C. for 1 h. The mixture was filtered and concentrated. The residue was purified by column chromatography (SiO₂, Petroleum ether/Ethyl acetate=10/1 to 2/1) to give tert-butyl 4-[2-[3-[2-bromo-4-(ethylsulfonylmethyl)phenoxy]phenoxy]ethoxy]piperidine-1-carboxylate (440 mg, 75% yield). ¹HNMR (400 MHz, CDCl₃) δ 1.44-1.45 (m, 3H), 1.50 (s, 9H), 1.86-1.92 (m, 2H), 2.98-2.99 (m, 2H), 3.12˜3.13 (m, 3H), 3.59 (m, 1H), 3.75-3.90 (m, 5H), 4.14 (s, 2H), 4.21 (s, 2H), 6.59-6.65 (m, 2H), 6.74-6.79 (m, 1H), 6.99 (d, J=8.52 Hz, 1H), 7.26-7.30 (m, 1H), 7.34˜7.35 (m, 1H), 7.72 (d, 0.1=2.12 Hz, 1H).

### 1.5 Synthesis of Compound 6

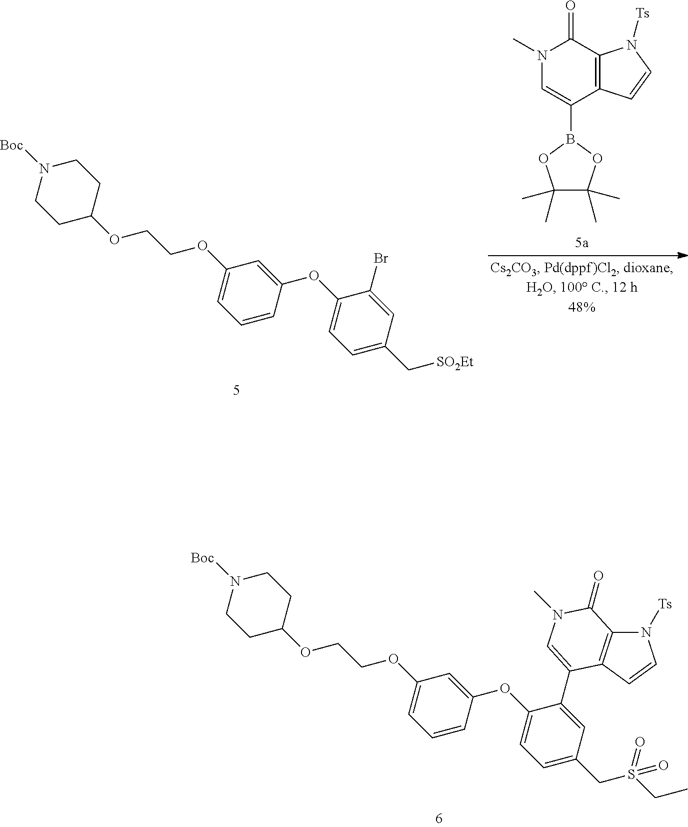

To a solution of tert-butyl 4-[2-[3-[2-bromo-4-(ethylsulfonylmethyl)phenoxy]phenoxy]ethoxy]piperidine-1-carboxylate (400 mg, 668 μmol, 1 equiv) in dioxane (8 mL) was added Cs₂CO₃ (653 mg, 2 mol, 3 equiv) and Pd(dppf)Cl₂ (98 mg, 134 mol, 0.2 equiv), 6-methyl-1-(p-tolylsulfonyl)-4-(4,4,5,5-tetramethyl-1,3,2-dioxaborolan-2-yl)pyrrolo[2,3-c]pyridin-7-one (343 mg, 802 mol, 1.2 equiv). The mixture was stirred at 100° C. for 12 h. The residue was purified by column chromatography (SiO₂, Petroleum ether/Ethyl acetate=10/1 to 1/1) to give tert-butyl 4-[2-[3-[4-(ethylsulfonylmethyl)-2-[6-methyl-7-oxo-1-(p-tolylsulfonyl)pyrrolo[2,3-c]pyridin-4-yl]phenoxy]phenoxy]ethoxy]piperidine-1-carboxylate (264 mg, 48% yield). ¹HNMR (400 MHz, DMSO-d₆) δ 1.23˜1.24 (m, 3H) 1.38 (s, 9H), 1.72-1.83 (m, 2H), 2.36-2.39 (m, 3H), 2.92-3.12 (m, 4H), 3.32 (s, 2H), 3.38-3.47 (m, 4H), 3.47-3.53 (m, 1H), 3.57-3.65 (m, 2H), 3.67-3.72 (m, 2H), 3.97˜3.98 (m, 1H), 4.50 (s, 2H), 6.44-6.51 (m, 2H), 6.57 (d, J=3.56 Hz, 1H), 6.64˜6.65 (m, 1H), 7.01 (d, J=8.44 Hz, 1H), 7.18˜7.19 (m, 1H), 7.35-7.53 (m, 6H), 7.96-8.00 (m, 2H).

### 1.6 Synthesis of Compound 7

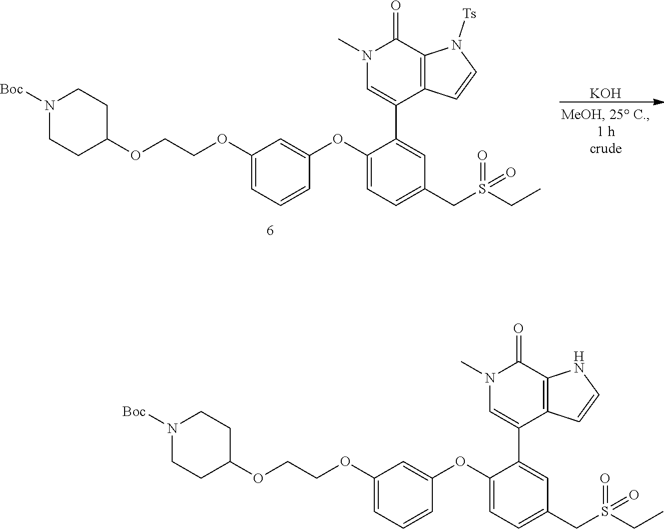 [SMILES: CCS(=O)(=O)Cc1ccc(Oc2cccc(OCCOC3CCN(C(=O)OC(C)(C)C)CC3)c2)c(-c2cn(C)c(=O)c3c2ccn3S(=O)(=O)c2ccc(C)cc2)c1 ; "KOH" ; CCS(=O)(=O)Cc1ccc(Oc2cccc(OCCOC3CCN(C(=O)OC(C)(C)C)CC3)c2)c(-c2cn(C)c(=O)c3[nH]ccc23)c1]

To a solution of tert-butyl 4-[2-[3-[4-(ethylsulfonylmethyl)-2-[6-methyl-7-oxo-1-(p-tolylsulfonyl)pyrrolo[2,3-c]pyridin-4-yl]phenoxy]phenoxy]ethoxy]piperidine-1-carboxylate (244 mg, 298 μmol, 1 equiv) in MeOH (3 mL) was added KOH (50 mg, 893 μmol, 3 equiv). The mixture was stirred at 25° C. for 1 h. The reaction mixture was filtered and concentrated under reduced pressure to give crude product tert-butyl 4-[2-[3-[4-(ethylsulfonylmethyl)-2-(6-methyl-7-oxo-1H-pyrrolo[2,3-c]pyridin-4-yl)phenoxy]phenoxy]ethoxy]piperidine-1-carboxylate (200 mg) was a yellow solid.

### 1.7 Synthesis of Compound 8

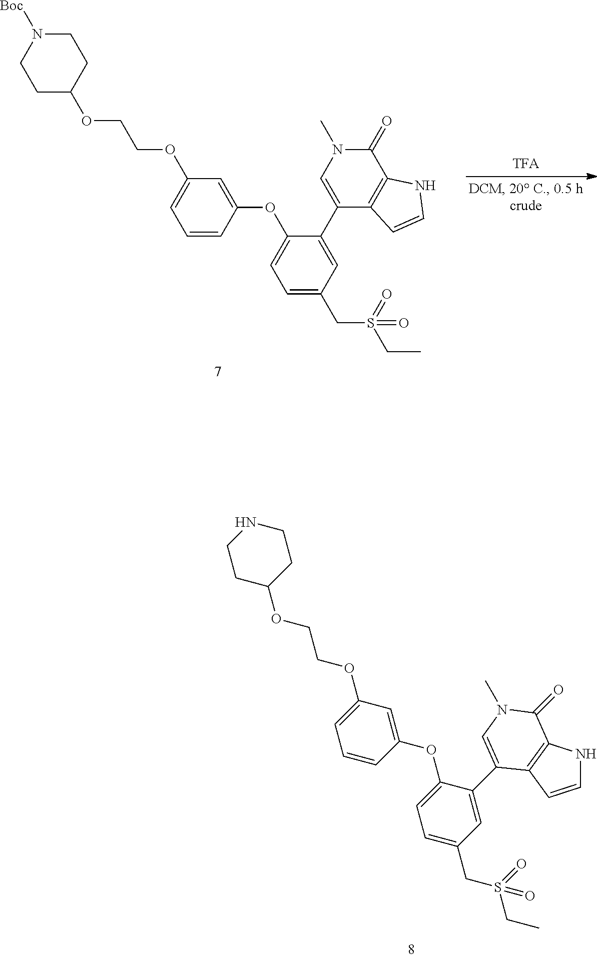 [SMILES: CCS(=O)(=O)Cc1ccc(Oc2cccc(OCCOC3CCN(C(=O)OC(C)(C)C)CC3)c2)c(-c2cn(C)c(=O)c3[nH]ccc23)c1 ; CCS(=O)(=O)Cc1ccc(Oc2cccc(OCCOC3CCNCC3)c2)c(-c2cn(C)c(=O)c3[nH]ccc23)c1]

To a solution of tert-butyl 4-[2-[3-[4-(ethylsulfonylmethyl)-2-(6-methyl-7-oxo-1H-pyrrolo[2,3-c]pyridin-4-yl)phenoxy]phenoxy]ethoxy]piperidine-1-carboxylate (60 mg, 90 μmol, 1 equiv) in DCM (4 mL) was added TFA (2 mL). The mixture was stirred at 20° C. for 0.5 h. The mixture was filtered and concentrated to give crude product 4-[5-(ethylsulfonyl-methyl)-2-[3-[2-(4-piperidyloxy)ethoxy]phenoxy]phenyl]-6-methyl-1H-pyrrolo[2,3-c]pyridin-7-one (50 mg, TFA salt) was a yellow oil.

### 1.8 Synthesis of I-54

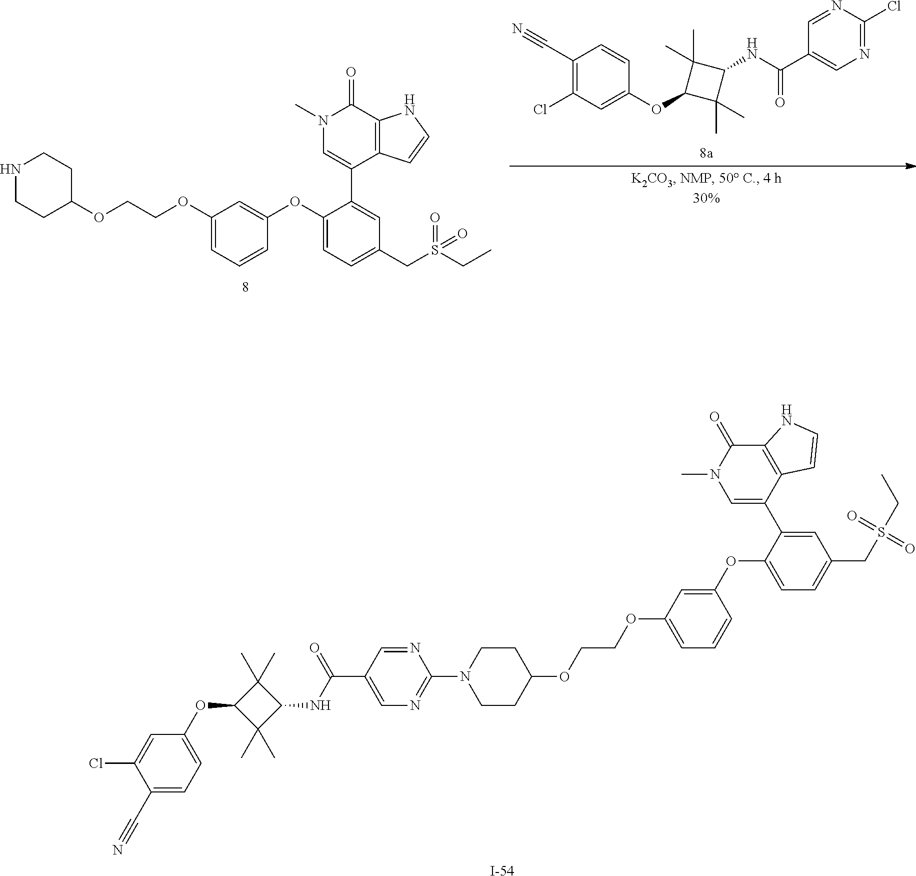

To a solution of 4-[5-(ethylsulfonylmethyl)-2-[3-[2-(4-piperidyloxy)ethoxy]phenoxy]phenyl]-6-methyl-1H-pyrrolo[2,3-c]pyridin-7-one (50 mg, 74 μmol, 1 equiv, TFA salt) in NMP (1 mL) was added K₂CO₃ (31 mg, 221 μmol, 3 equiv) and 2-chloro-N-[3-(3-chloro-4-cyano-phenoxy)-2,2,4,4-tetramethyl-cyclobutyl]pyrimidine-5-carboxamide (31 mg, 74 gmol, 1 equiv). The mixture was stirred at 50° C. for 4 h and concentrated. The residue was purified by prep-HPLC (column: Unisil 3-100 C18 Ultra 150 mm*50 mm*34 μm; mobile phase: [water(FA)-ACN]; B %: 53%-83%, 7 min) to give N-[3-(3-chloro-4-cyano-phenoxy)-2,2,4,4-tetramethyl-cyclobutyl]-2-[4-[2-[3-[4-(ethylsulfonylmethyl)-2-(6-methyl-7-oxo-1H-pyrrolo [2,3-c]pyridin-4-yl)phenoxy]phenoxy]ethoxy]-1-piperidyl]pyrimidine-5-carboxamide (21 mg, 30% yield). ¹H NMR (400 MHz, DMSO-d₆) δ 1.11 (s, 6H), 1.20-1.26 (m, 9H), 1.38-1.47 (m, 2H), 1.82-1.91 (m, 2H), 3.06-3.08 (m, 2H), 3.40-3.56 (m, 6H), 3.66-3.67 (m, 1H), 3.70-3.77 (m, 2H), 3.99-4.06 (m, 3H), 4.20-4.30 (m, 3H), 4.51 (s, 2H), 6.33 (s, 1H), 6.44-6.54 (m, 2H), 6.65-6.66 (m, 1H), 6.98-7.07 (m, 2H), 7.14-7.22 (m, 2H), 7.26-7.31 (m, 2H), 7.38-7.39 (m, 1H), 7.60 (d, J=2.00 Hz, 1H), 7.71 (d, J=9.28 Hz, 1H), 7.90 (d, J=8.76 Hz, 1H), 8.76 (s, 2H), 11.96-12.08 (m, 1H).

### Example 9—Synthesis of N-((1r, 3r)-3-(3-chloro-4-cyanophenoxy)-2,2,4,4-tetramethylcyclobutyl)-2-(4-(2-(3-(4-(ethylsulfonyl)-2-(6-methyl-7-oxo-6,7-dihydro-1H-pyrrolo[2,3-c]pyridin-4-yl)phenoxy)phenoxy)ethoxy)piperidin-1-yl)pyrimidine-5-carboxamide (I-55)

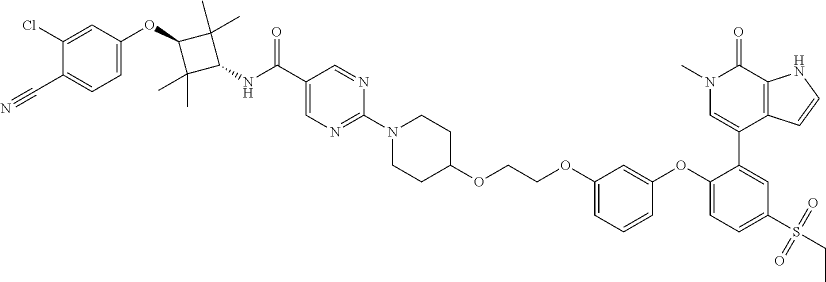 [SMILES: CCS(=O)(=O)c1ccc(Oc2cccc(OCCOC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1]

### 1.1 Synthesis of Compound 2

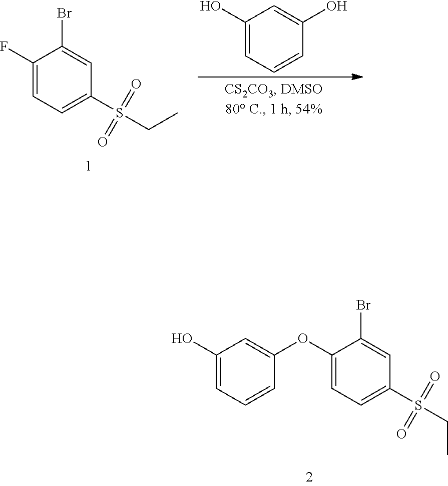 [SMILES: CCS(=O)(=O)c1ccc(F)c(Br)c1 ; Oc1cccc(O)c1 ; CCS(=O)(=O)c1ccc(Oc2cccc(O)c2)c(Br)c1]

To a solution of 2-bromo-4-ethylsulfonyl-1-fluoro-benzene (790 mg, 2.9 mol, 1 equiv) and benzene-1,3-diol (814 mg, 7.3 mol, 2.5 equiv) in DMSO (8 mL) was added Cs₂CO₃ (2.4 g, 7.3 μmol, 2.5 equiv). The mixture was stirred at 80° C. for 1 h and purified by prep-HPLC (column: Waters Xbridge C18 150 mm*50 mm*10 μm; mobile phase: [water (NH₄HCO₃)-ACN]; B %: 28%-58%, 10 min) to give3-(2-bromo-4-ethylsulfonyl-phenoxy) phenol (580 mg, 54% yield) as a yellow oil. ¹H NMR (400 MHz, MeOD): δ 8.16 (s, 1H), 7.89-7.73 (m, 1H), 7.33-7.18 (m, 1H), 7.10-6.94 (m, 1H), 6.75-6.64 (m, 1H), 6.56-6.40 (m, 2H), 3.26-3.23 (m, 2H), 1.31-1.14 (m, 3H).

### 1.2 Synthesis of Compound 4

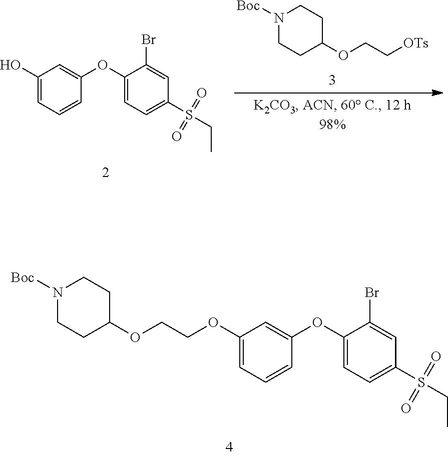 [SMILES: CCS(=O)(=O)c1ccc(Oc2cccc(O)c2)c(Br)c1 ; Cc1ccc(S(=O)(=O)OCCOC2CCN(C(=O)OC(C)(C)C)CC2)cc1 ; CCS(=O)(=O)c1ccc(Oc2cccc(OCCOC3CCN(C(=O)OC(C)(C)C)CC3)c2)c(Br)c1]

To a solution of tert-butyl 4-[2-(p-tolylsulfonyloxy) ethoxy]piperidine-1-carboxylate (559.1 mg, 1.4 μmol, 1 equiv) and 3-(2-bromo-4-ethylsulfonyl-phenoxy) phenol (500 mg, 1.40 mol, 1 equiv) in ACN (10 mL) was added Cs₂CO₃ (912.1 mg, 2.8 μmol, 2 equiv). The mixture was stirred at 80° C. for 1 h. The mixture was filtered and concentrated. The residue was purified by column chromatography (SiO₂, Petroleum ether/Ethyl acetate=10/1 to 2/1) to give tert-butyl 4-[2-[3-(2-bromo-4-ethylsulfonyl-phenoxy) phenoxy]ethoxy]piperidine-1-carboxylate (806 mg, 98% yield) as a yellow oil.

### 1.3 Synthesis of Compound 6

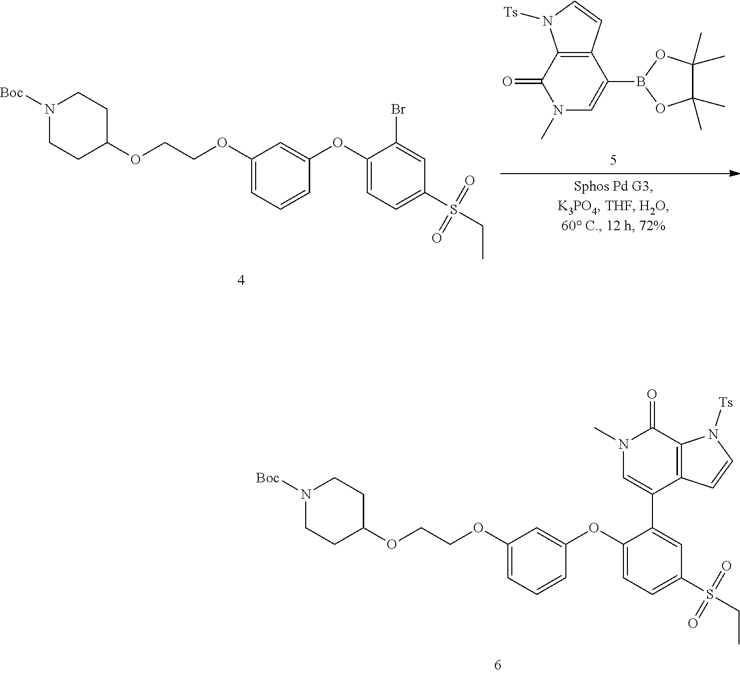 [SMILES: CCS(=O)(=O)c1ccc(Oc2cccc(OCCOC3CCN(C(=O)OC(C)(C)C)CC3)c2)c(Br)c1 ; Cc1ccc(S(=O)(=O)n2ccc3c(B4OC(C)(C)C(C)(C)O4)cn(C)c(=O)c32)cc1 ; CCS(=O)(=O)c1ccc(Oc2cccc(OCCOC3CCN(C(=O)OC(C)(C)C)CC3)c2)c(-c2cn(C)c(=O)c3c2ccn3S(=O)(=O)c2ccc(C)cc2)c1]

To a solution of tert-butyl 4-[2-[3-(2-bromo-4-ethylsulfonyl-phenoxy)phenoxy]ethoxy]piperidine-1-carboxylate (125 mg, 214 μmol, 1 equiv) and 6-methyl-1-(p-tolylsulfonyl)-4-(4,4,5,5-tetramethyl-1,3,2-dioxaborolan-2-yl)pyrrolo[2,3-c]pyridin-7-one (110 mg, 257 μmol, 1.2 equiv) in H₂O (0.5 mL) and THF (2.5 mL) was added dicyclohexyl-[2-(2,6-dimethoxyphenyl)phenyl]phosphane;methanesulfonate;(2-phenylanilino) palladium(1+) (17 mg, 21 mol, 0.1 equiv) and K₃PO₄ (136 mg, 642 μmol, 3 equiv). The mixture was stirred at 60° C. for 12 h. The mixture was purified by prep-TLC (SiO₂, DCM: MeOH=20:1) to give tert-butyl 4-[2-[3-[4-ethylsulfonyl-2-[6-methyl-7-oxo-1-(p-tolylsulfonyl)pyrrolo[2,3-c]pyridin-4-yl]phenoxy]phenoxy]ethoxy]piperidine-1-carboxylate (125 mg, 72% yield) as a yellow oil.

### 1.4 Synthesis of Compound 7

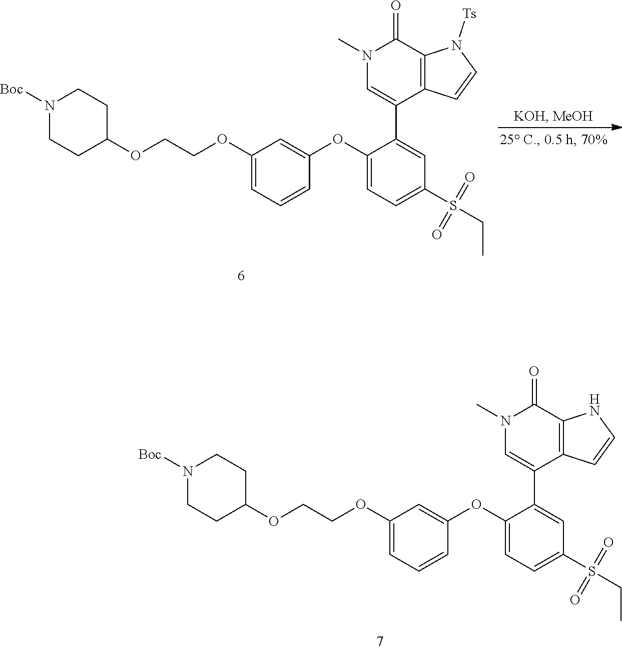

To a solution of tert-butyl 4-[2-[3-[4-ethylsulfonyl-2-[6-methyl-7-oxo-1-(p-tolylsulfonyl)pyrrolo[2,3-c]pyridin-4-yl]phenoxy]phenoxy]ethoxy]piperidine-1-carboxylate (110 mg, 136 μmol, 1 equiv) in MeOH (2 mL) was added KOH (23 mg, 409 mol, 3 equiv). The mixture was stirred at 25° C. for 0.5 h. The residue was purified by prep-TLC (SiO₂, Petroleum ether: Ethyl acetate=1:1) to give tert-butyl 4-[2-[3-[4-ethylsulfonyl-2-(6-methyl-7-oxo-1H-pyrrolo [2, 3-c]pyridin-4-yl) phenoxy]phenoxy]ethoxy]piperidine-1-carboxylate (63 mg, 70% yield) as a yellow oil.

### 1.5 Synthesis of Compound 8

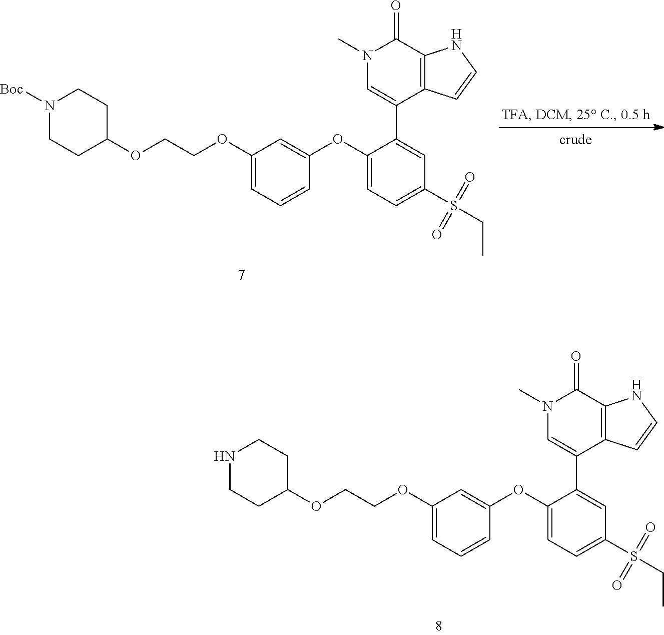 [SMILES: CCS(=O)(=O)c1ccc(Oc2cccc(OCCOC3CCN(C(=O)OC(C)(C)C)CC3)c2)c(-c2cn(C)c(=O)c3[nH]ccc23)c1 ; CCS(=O)(=O)c1ccc(Oc2cccc(OCCOC3CCNCC3)c2)c(-c2cn(C)c(=O)c3[nH]ccc23)c1]

To a solution of tert-butyl 4-[2-[3-[4-ethylsulfonyl-2-(6-methyl-7-oxo-1H-pyrrol [2, 3-c]pyridin-4-yl) phenoxy]phenoxy]ethoxy]piperidine-1-carboxylate (50 mg, 77 mol, 1 equiv) in DCM (1 mL) was added TFA (1 mL). The mixture was stirred at 25° C. for 0.5 h. The mixture was concentrated to give crude 4-[5-ethylsulfonyl-2-[3-[2-(4-piperidyloxy) ethoxy]phenoxy]phenyl]-6-methyl-1H-pyrrolo [2, 3-c]pyridin-7-one (51 mg) as a yellow oil.

### 1.6 Synthesis of I-55

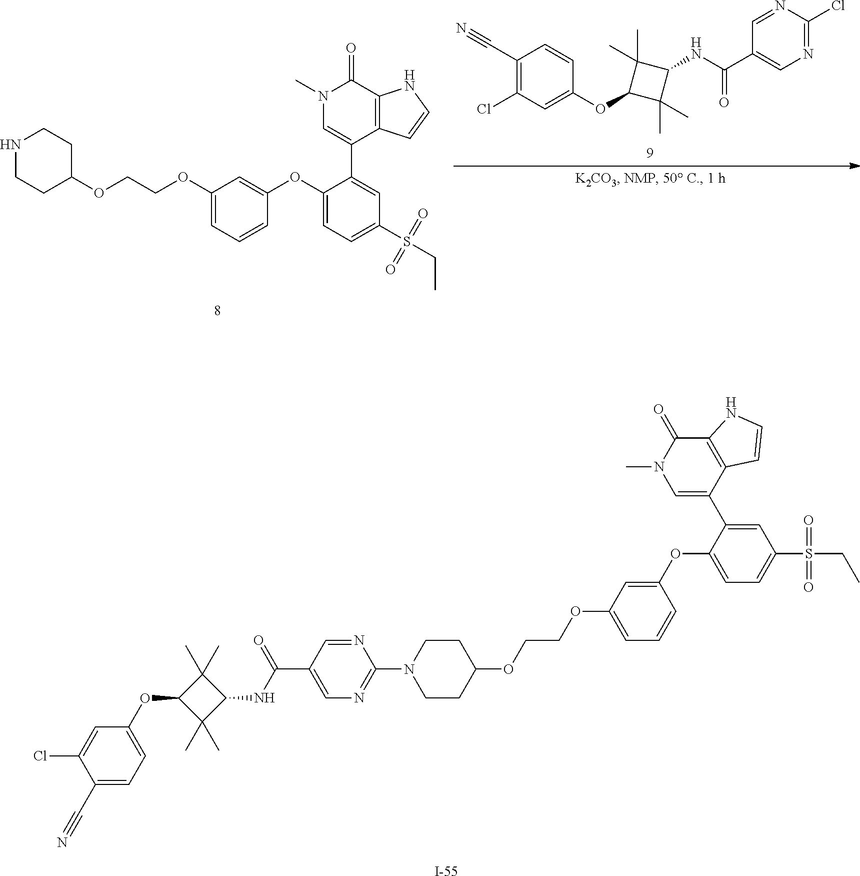

To a solution of 4-[5-ethylsulfonyl-2-[3-[2-(4-piperidyloxy)ethoxy]phenoxy]phenyl]-6-methyl-1H-pyrrolo[2,3-c]pyridin-7-one (51 mg, 77 μmol, 1 equiv, TFA salt) and 2-chloro-N-[3-(3-chloro-4-cyano-phenoxy)-2,2,4,4-tetramethyl-cyclobutyl]pyrimidine-5-carboxamide (32 mg, 77 mol, 1 equiv) in NMP (1 mL) was added K₂CO₃ (53 mg, 383 mol, 5 equiv). The mixture was stirred at 50° C. for 1 h. The residue was purified by prep-HPLC (column: Unisil 3-100 C18 Ultra 150 mm*50 μm*3 μm; mobile phase: [water(FA)-ACN]; B %: 54%-84%, 7 min) to give N-[3-(3-chloro-4-cyano-phenoxy)-2,2,4,4-tetramethyl-cyclobutyl]-2-[4-[2-[3-[4-ethylsulfonyl-2-(6-methyl-7-oxo-1H-pyrrolo[2,3-c]pyridin-4-yl)phenoxy]phenoxy]ethoxy]-1-piperidyl]pyrimidine-5-carboxamide (30.7 mg, 42% yield) as a yellow solid. ¹H NMR (400 MHz, MeOD): δ 8.69 (s, 2H), 8.05-7.99 (m, 1H), 7.88-7.82 (m, 1H), 7.76-7.67 (m, 1H), 7.37-7.31 (m, 2H), 7.23 (t, J=8.0 Hz, 1H), 7.19-7.11 (m, 2H), 7.00-6.95 (m, 1H), 6.78-6.71 (m, 1H), 6.62-6.55 (m, 2H), 6.37-6.31 (m, 1H), 4.27-4.18 (m, 3H), 4.14-4.05 (m, 3H), 3.85-3.80 (m, 2H), 3.72-3.57 (m, 6H), 3.29-3.23 (m, 2H), 1.95-1.84 (m, 2H), 1.62-1.50 (m, 2H), 1.29-1.25 (m, 9H), 1.21 (s, 6H).

### Example 10—Synthesis of N-((1r, 3r)-3-(3-chloro-4-cyanophenoxy)-2,2,4,4-tetramethylcyclobutyl)-2-(4-(2-(3-(4-(2-hydroxypropan-2-yl)-2-(6-methyl-7-oxo-6,7-dihydro-1H-pyrrolo[2,3-c]pyridin-4-yl)phenoxy)phenoxy)ethoxy)piperidin-1-yl)pyrimidine-5-carboxamide (I-56)

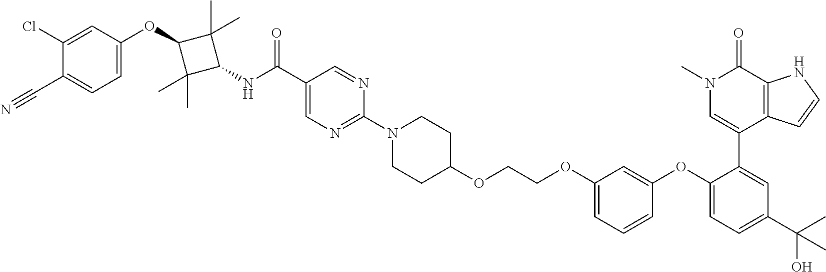 [SMILES: Cn1cc(-c2cc(C(C)(C)O)ccc2Oc2cccc(OCCOC3CCN(c4ncc(C(=O)N[C@H]5C(C)(C)[C@H](Oc6ccc(C#N)c(Cl)c6)C5(C)C)cn4)CC3)c2)c2cc[nH]c2c1=O]

### 1.1 Synthesis of Compound 2

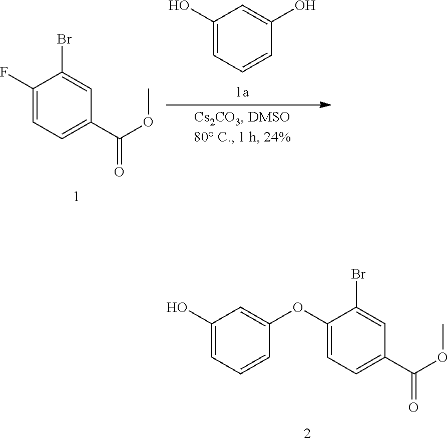 [SMILES: COC(=O)c1ccc(F)c(Br)c1 ; Oc1cccc(O)c1 ; COC(=O)c1ccc(Oc2cccc(O)c2)c(Br)c1]

To a solution of benzene-1,3-diol (3.54 g, 32 μmol, 2.5 equiv) and methyl 3-bromo-4-fluoro-benzoate (3 g, 12.8 μmol, 1 equiv) in DMSO (7 mL) was added Cs₂CO₃ (10.4 g, 32 μmol, 2.5 equiv). The mixture was stirred at 80° C. for 1 h. The residue was purified prep-HPLC (column: Phenomenex luna C18 150 mm*40 mm*15 μm; mobile phase: [water(FA)-ACN]; B %: 43%-73%, 10 min) to give methyl 3-bromo-4-(3-hydroxyphenoxy)benzoate (1 g, 24% yield).

### 1.2 Synthesis of Compound 3

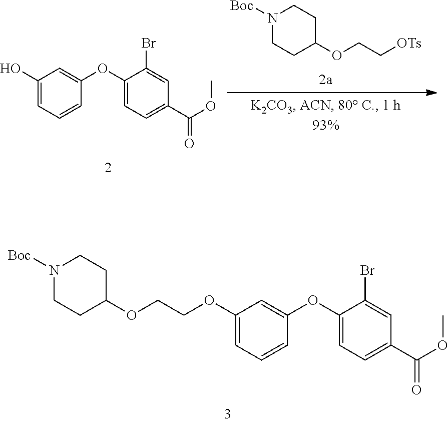 [SMILES: COC(=O)c1ccc(Oc2cccc(O)c2)c(Br)c1 ; Cc1ccc(S(=O)(=O)OCCOC2CCN(C(=O)OC(C)(C)C)CC2)cc1 ; COC(=O)c1ccc(Oc2cccc(OCCOC3CCN(C(=O)OC(C)(C)C)CC3)c2)c(Br)c1]

To a solution of tert-butyl 4-[2-(p-tolylsulfonyloxy) ethoxy]piperidine-1-carboxylate (618 mg, 1.5 μmol, 1 equiv) and methyl 3-bromo-4-(3-hydroxyphenoxy) benzoate (500 mg, 1.5 μmol, 1 equiv) in ACN (10 mL) was added Cs₂CO₃ (1.26 g, 3.8 μmol, 2.5 equiv). The mixture was stirred at 80° C. for 1 h. The mixture was purified by column chromatography (SiO₂, Petroleum ether/Ethyl acetate=10/1 to 2/1) to give tert-butyl 4-[2-[3-(2-bromo-4-methoxycarbonyl-phenoxy) phenoxy]ethoxy]piperidine-1-carboxylate (800 mg, 93% yield) as a yellow oil.

### 1.3 Synthesis of Compound 4

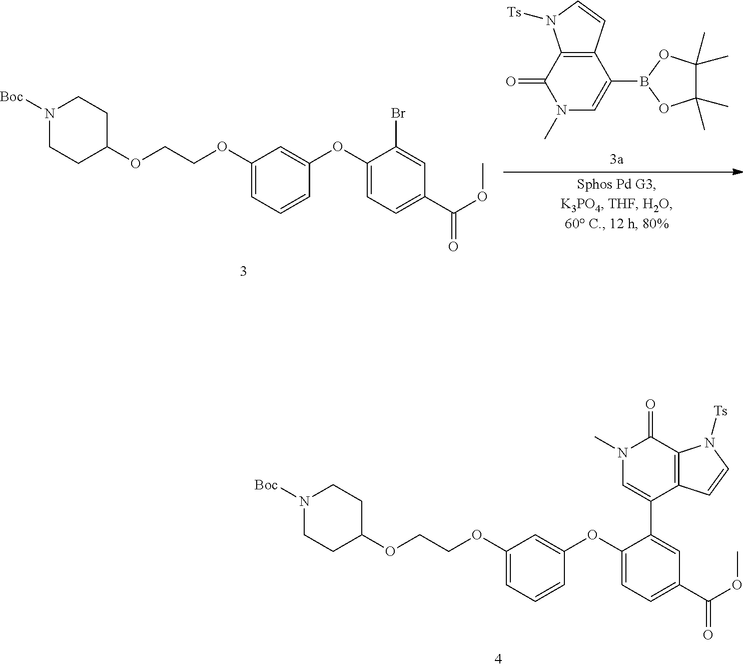 [SMILES: COC(=O)c1ccc(Oc2cccc(OCCOC3CCN(C(=O)OC(C)(C)C)CC3)c2)c(Br)c1 ; Cc1ccc(S(=O)(=O)n2ccc3c(B4OC(C)(C)C(C)(C)O4)cn(C)c(=O)c32)cc1 ; COC(=O)c1ccc(Oc2cccc(OCCOC3CCN(C(=O)OC(C)(C)C)CC3)c2)c(-c2cn(C)c(=O)c3c2ccn3S(=O)(=O)c2ccc(C)cc2)c1]

To a solution of 6-methyl-1-(p-tolylsulfonyl)-4-(4,4,5,5-tetramethyl-1,3,2-dioxaborolan-2-yl)pyrrolo[2,3-c]pyridin-7-one (100 mg, 233 mol, 1.2 equiv) and tert-butyl 4-[2-[3-(2-bromo-4-methoxycarbonyl-phenoxy)phenoxy]ethoxy]piperidine-1-carboxylate (107 mg, 194 μmol, 1 equiv) in THF (5 mL) and H₂O (1 mL) was added K₃PO₄ (124 mg, 583 μmol, 3 equiv) and SPhos Pd G3 (15 mg, 19 μmol, 0.1 equiv). The mixture was stirred at 60° C. for 12 h. The mixture was purified by prep-TLC (SiO₂, DCM: MeOH=10:1) to give tert-butyl 4-[2-[3-[4-methoxycarbonyl-2-[6-methyl-7-oxo-1-(p-tolylsulfonyl)pyrrolo[2,3-c]pyridin-4-yl]phenoxy]phenoxy]ethoxy]piperidine-1-carboxylate (140 mg, 80% yield).

### 1.4 Synthesis of Compound 5

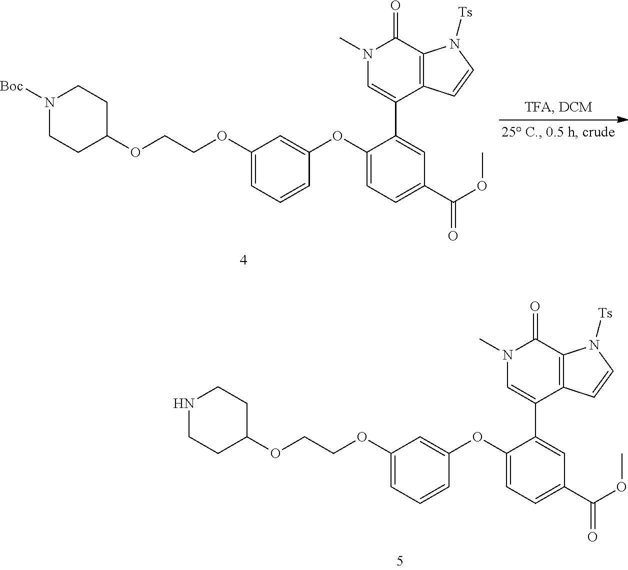 [SMILES: COC(=O)c1ccc(Oc2cccc(OCCOC3CCN(C(=O)OC(C)(C)C)CC3)c2)c(-c2cn(C)c(=O)c3c2ccn3S(=O)(=O)c2ccc(C)cc2)c1 ; COC(=O)c1ccc(Oc2cccc(OCCOC3CCNCC3)c2)c(-c2cn(C)c(=O)c3c2ccn3S(=O)(=O)c2ccc(C)cc2)c1]

To a solution of tert-butyl 4-[2-[3-[4-methoxycarbonyl-2-[6-methyl-7-oxo-1-(p-tolylsulfonyl)pyrrolo[2,3-c]pyridin-4-yl]phenoxy]phenoxy]ethoxy]piperidine-1-carboxylate (80 mg, 104 mol, 1 equiv) in DCM (1 mL) was added TFA (0.3 mL). The mixture was stirred at 25° C. for 0.5 h. The reaction mixture was concentrated to give methyl 3-[6-methyl-7-oxo-1-(p-tolylsulfonyl) pyrrolo [2, 3-c]pyridin-4-yl]-4-[3-[2-(4-piperidyloxy) ethoxy]phenoxy]benzoate (81.4 mg, crude, TFA salt) as a yellow oil.

### 1.5 Synthesis of Compound 6

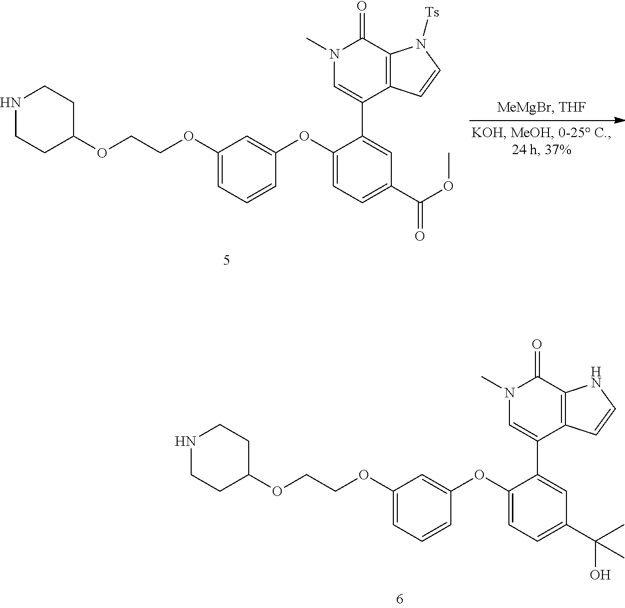 [SMILES: COC(=O)c1ccc(Oc2cccc(OCCOC3CCNCC3)c2)c(-c2cn(C)c(=O)c3c2ccn3S(=O)(=O)c2ccc(C)cc2)c1 ; Cn1cc(-c2cc(C(C)(C)O)ccc2Oc2cccc(OCCOC3CCNCC3)c2)c2cc[nH]c2c1=O]

To a solution of methyl 3-[6-methyl-7-oxo-1-(p-tolylsulfonyl)pyrrolo[2,3-c]pyridin-4-yl]-4-[3-[2-(4-piperidyloxy)ethoxy]phenoxy]benzoate (81 mg, 103 mol, 1 equiv, TFA salt) in THF (2 mL) was added MeMgBr (3 M, 171 μL, 5 equiv) at 0° C. The mixture was stirred at 25° C. for 12 h and KOH (δ mg, 103 μmol, 1 equiv) was added. The mixture was stirred at 25° C. for 12 h and purified by prep-HPLC (column: Waters Xbridge 150 mm*25 mm*5 μm; mobile phase: [water (NH₄HCO₃)-ACN]; B %: 25%-55%, 9 min) to give 4-[5-(1-hydroxy-1-methyl-ethyl)-2-[3-[2-(4-piperidyloxy)ethoxy]phenoxy]phenyl]-6-methyl-1H-pyrrolo[2,3-c]pyridin-7-one (20 mg, 37% yield) as a yellow oil.

### 1.6 Synthesis of I-56

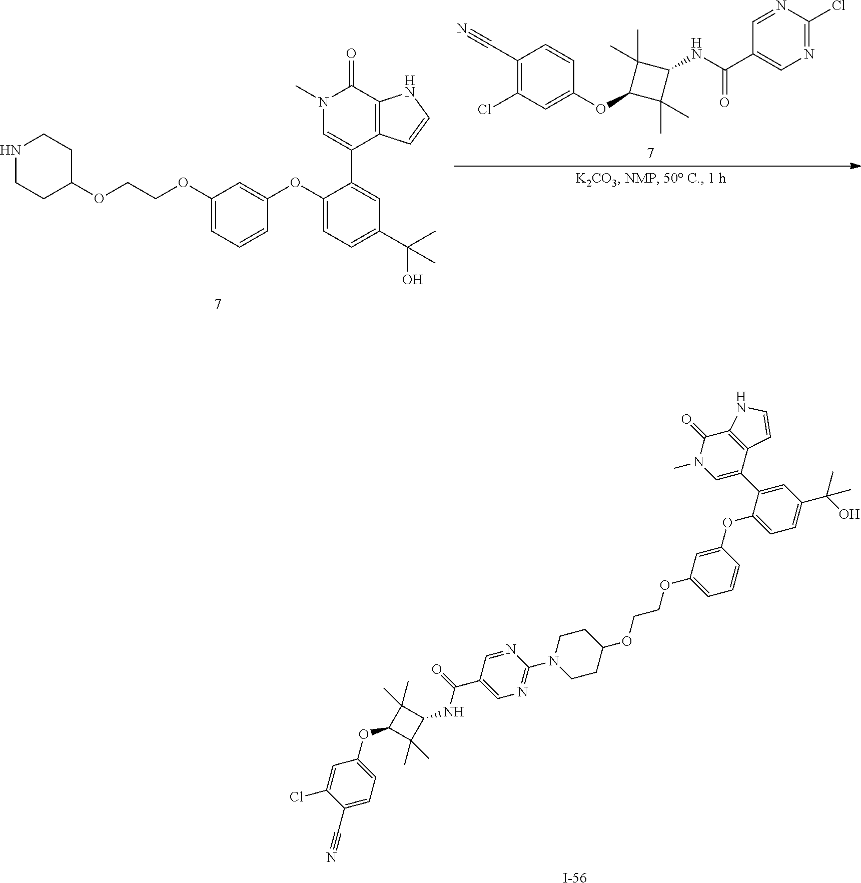

To a solution of 2-chloro-N-[3-(3-chloro-4-cyano-phenoxy)-2,2,4,4-tetramethyl-cyclobutyl]pyrimidine-5-carboxamide (8 mg, 19 μmol, 1 equiv) and 4-[5-(1-hydroxy-1-methyl-ethyl)-2-[3-[2-(4-piperidyloxy)ethoxy]phenoxy]phenyl]-6-methyl-1H-pyrrolo[2,3-c]pyridin-7-one (10 mg, 19 mol, 1 equiv) in NMP (1 mL) was added K₂CO₃ (13 mg, 97 μmol, 5 equiv). The mixture was stirred at 50° C. for 1 h. The residue was purified by prep-HPLC (column: Unisil 3-100 C18 Ultra 150 mm*50 mm*3 μm; mobile phase: [water(FA)-ACN]; B %: 55%-85%, 7 min) to give N-[3-(3-chloro-4-cyano-phenoxy)-2,2,4,4-tetramethyl-cyclobutyl]-2-[4-[2-[3-[4-(1-hydroxy-1-methyl-ethyl)-2-(6-methyl-7-oxo-1H-pyrrolo[2,3-c]pyridin-4-yl)phenoxy]phenoxy]ethoxy]-1-piperidyl]pyrimidine-5-carboxamide (2.93 mg, 16% yield). ¹H NMR (400 MHz, MeOD) δ 8.72 (s, 2H), 7.75-7.66 (m, 2H), 7.55-7.48 (m, 1H), 7.32 (d, J=2.8 Hz, 1H), 7.16 (s, 1H), 7.13 (d, J=2.4 Hz, 1H), 7.10-7.03 (m, 2H), 6.99-6.96 (m, 1H), 6.55-6.50 (m, 1H), 6.38-6.32 (m, 3H), 4.60-4.55 (m, 1H), 4.29-4.22 (m, 3H), 4.13 (s, 1H), 4.01-3.96 (m, 2H), 3.82-3.76 (m, 2H), 3.75-3.67 (m, 1H), 3.63-3.56 (m, 5H), 1.95-1.87 (m, 2H), 1.62-1.55 (m, 8H), 1.28 (s, 6H), 1.21 (s, 6H).

### Example 11—Synthesis of 2-(4-(2-(3-(4-acetamido-2-(6-methyl-7-oxo-6,7-dihydro-1H-pyrrolo [2,3-c]pyridin-4-yl)phenoxy)phenoxy)ethoxy)piperidin-1-yl)-N-((1r, 3r)-3-(3-chloro-4-cyanophenoxy)-2,2,4,4-tetramethylcyclobutyl)pyrimidine-5-carboxamide (I-57)

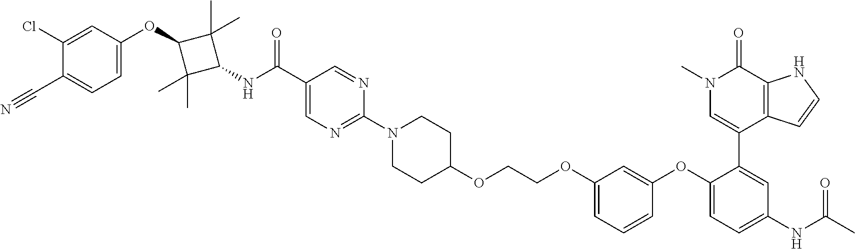 [SMILES: CC(=O)Nc1ccc(Oc2cccc(OCCOC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1]

### 1.1 Synthesis of Compound 2

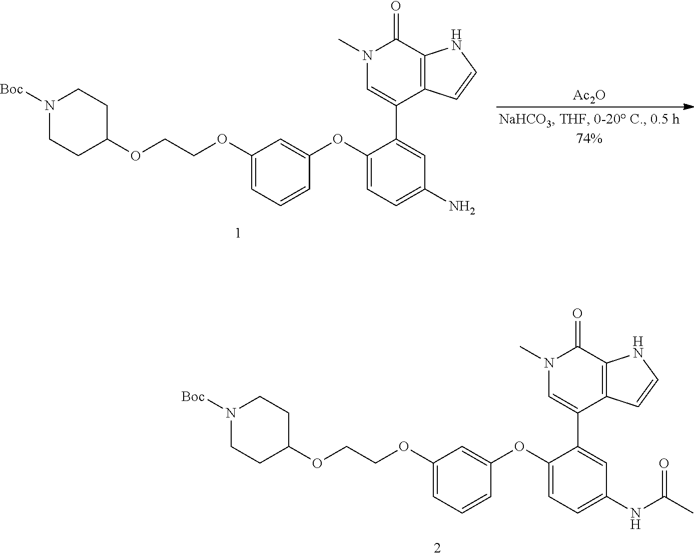 [SMILES: "Ac2O" ; Cn1cc(-c2cc(N)ccc2Oc2cccc(OCCOC3CCN(C(=O)OC(C)(C)C)CC3)c2)c2cc[nH]c2c1=O ; CC(=O)Nc1ccc(Oc2cccc(OCCOC3CCN(C(=O)OC(C)(C)C)CC3)c2)c(-c2cn(C)c(=O)c3[nH]ccc23)c1]

To a solution of tert-butyl 4-[2-[3-[4-amino-2-(6-methyl-7-oxo-1H-pyrrolo[2,3-c]pyridin-4-yl)phenoxy]phenoxy]ethoxy]piperidine-1-carboxylate (50 mg, 87 mol, 1.0 equiv) in THF (1 mL) was added NaHCO₃ (1 mL) and Ac₂O (9 mg, 87 μmol, 1.0 equiv) at 0° C. The mixture was stirred at 20° C. for 0.5 h. The mixture was diluted with water (2 mL) and extracted with DCM (3×3 mL). The combined organic layers were dried over sodium sulfate, filtered and concentrated. The residue was purified by prep-TLC (SiO₂, DCM: MeOH=10:1) to give tert-butyl 4-[2-[3-[4-acetamido-2-(6-methyl-7-oxo-1H-pyrrolo[2,3-c]pyridin-4-yl)phenoxy]phenoxy]ethoxy]piperidine-1-carboxylate (40 mg, 74% yield) as a colorless oil.

### 1.2 Synthesis of Compound 3

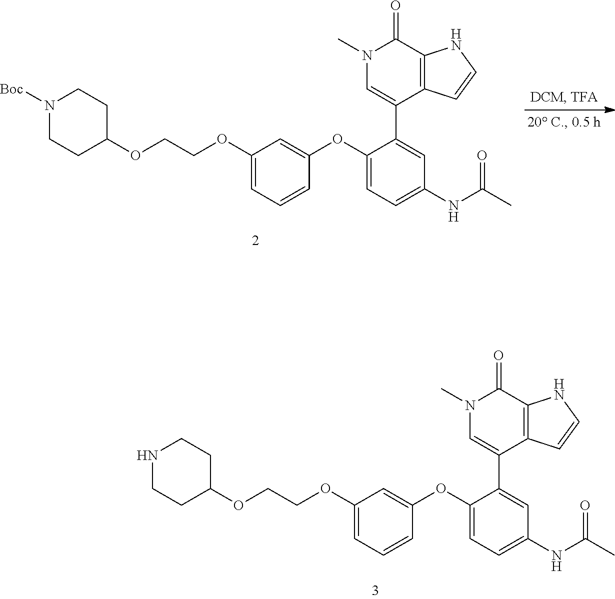 [SMILES: CC(=O)Nc1ccc(Oc2cccc(OCCOC3CCN(C(=O)OC(C)(C)C)CC3)c2)c(-c2cn(C)c(=O)c3[nH]ccc23)c1 ; CC(=O)Nc1ccc(Oc2cccc(OCCOC3CCNCC3)c2)c(-c2cn(C)c(=O)c3[nH]ccc23)c1]

To a solution of tert-butyl 4-[2-[3-[4-acetamido-2-(6-methyl-7-oxo-1H-pyrrolo[2,3-c]pyridin-4-yl)phenoxy]phenoxy]ethoxy]piperidine-1-carboxylate (40 mg, 65 mol, 1.0 equiv) in DCM (1.5 mL) and TFA (0.5 mL). The mixture was stirred at 20° C. for 0.5 h. The solution was concentrated under reduced pressure to give N-[3-(6-methyl-7-oxo-1H-pyrrolo [2,3-c]pyridin-4-yl)-4-[3-[2-(4-piperidyloxy)ethoxy]phenoxy]phenyl]acetamide (40 mg, crude, TFA salt) as a yellow oil.

### 1.3 Synthesis of I-57

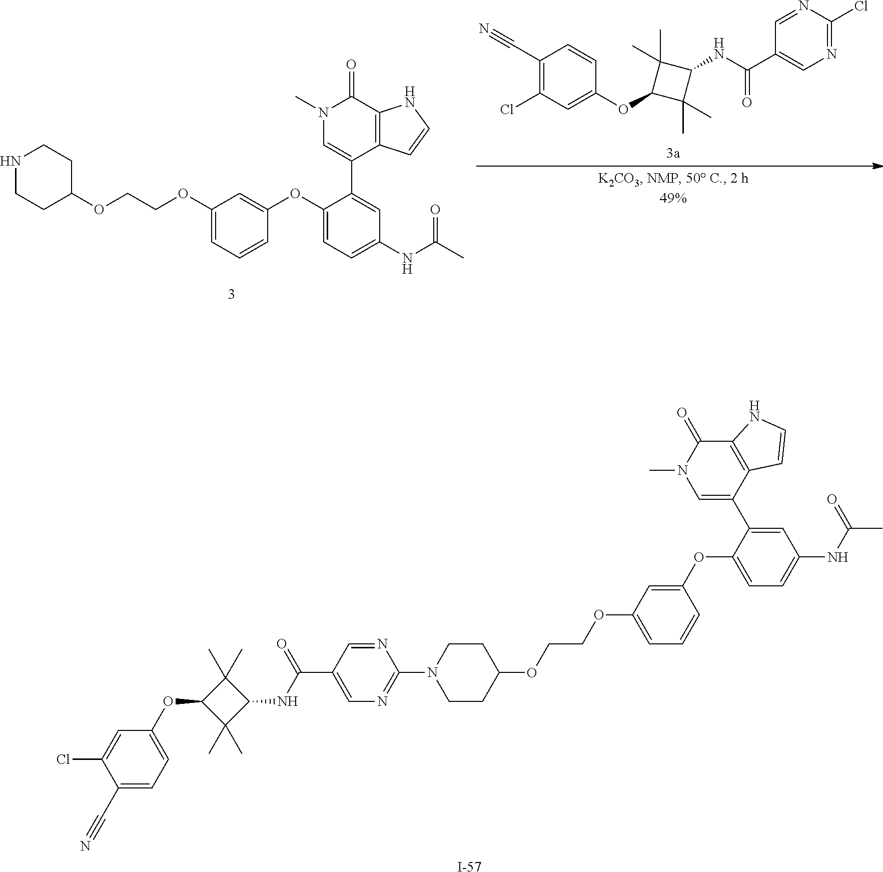

To a solution of N-[3-(6-methyl-7-oxo-1H-pyrrolo[2,3-c]pyridin-4-yl)-4-[3-[2-(4-piperidyloxy)ethoxy]phenoxy]phenyl]acetamide (40 mg, 63 μmol, 1.0 equiv) in NMP (1 mL) was added K₂CO₃ (26 mg, 190 μmol, 3.0 equiv) and 2-chloro-N-[3-(3-chloro-4-cyano-phenoxy)-2,2,4,4-tetramethyl-cyclobutyl]pyrimidine-5-carboxamide (26 mg, 63 mol, 1.0 equiv). The mixture was stirred at 50° C. for 2 h and concentrated. The residue was purified by prep-HPLC (column: Waters Xbridge 150 mm*25 mm*5 μm; mobile phase: [water(NH₄HCO₃)-ACN]; B %: 50%-80%, 9 min) to give 2-[4-[2-[3-[4-acetamido-2-(6-methyl-7-oxo-1H-pyrrolo[2,3-c]pyridin-4-yl)phenoxy]phenoxy]ethoxy]-1-piperidyl]-N-[3-(3-chloro-4-cyano-phenoxy)-2,2,4,4-tetramethyl-cyclobutyl]pyrimidine-5-carboxamide (28 mg, 49% yield) as a white solid. ¹H NMR (400 MHz, DMSO-d₆) δ 12.03 (s, 1H), 10.04 (s, 1H), 8.75 (s, 2H), 7.90 (d, J=8.8 Hz, 1H), 7.82-7.77 (m, 1H), 7.75-7.69 (m, 1H), 7.59-7.53 (m, 1H), 7.29-7.23 (m, 2H), 7.22-7.17 (m, 1H), 7.11 (t, J=8.0 Hz, 1H), 7.06-6.97 (m, 2H), 6.59-6.50 (m, 1H), 6.41-6.33 (m, 2H), 6.30-6.26 (m, 1H), 4.28 (s, 1H), 4.26-4.18 (m, 2H), 4.05-4.01 (m, 1H), 3.99-3.93 (m, 2H), 3.73-3.68 (m, 2H), 3.67-3.59 (m, 2H), 3.48 (s, 4H), 2.04 (s, 3H), 1.90-1.83 (m, 2H), 1.44-1.35 (m, 2H), 1.21 (s, 6H), 1.10 (s, 6H).

### Example 12—Synthesis of N-((1r, 3r)-3-(3-chloro-4-cyanophenoxy)-2,2,4,4-tetramethylcyclobutyl)-2-(4-(2-(3-(2-(6-methyl-7-oxo-6,7-dihydro-1H-pyrrolo[2,3-c]pyridin-4-yl)-4-propionamidophenoxy)phenoxy)ethoxy)piperidin-1-yl)pyrimidine-5-carboxamide (I-59)

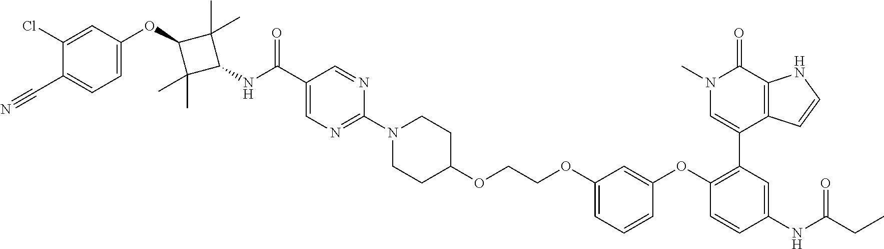 [SMILES: CCC(=O)Nc1ccc(Oc2cccc(OCCOC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1]

### 1.1 Synthesis of Compound 2

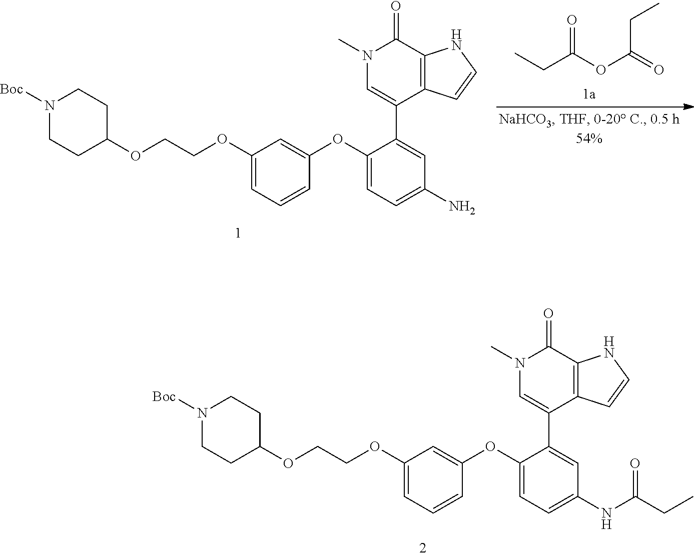

To a solution of tert-butyl 4-[2-[3-[4-amino-2-(6-methyl-7-oxo-1H-pyrrolo[2,3-c]pyridin-4-yl)phenoxy]phenoxy]ethoxy]piperidine-1-carboxylate (50 mg, 87 mol, 1.0 equiv) in THF (1 mL) was added NaHCO₃ (1 mL) and propanoyl propanoate (11 mg, 87 mol, 11 μL, 1.0 equiv) at 0° C. The mixture was stirred at 20° C. for 0.5 h. The mixture was diluted with water (2 mL) and extracted with DCM (3×3 mL). The combined organic layers were dried over sodium sulfate, filtered and concentrated. The residue was purified by prep-TLC (SiO₂, DCM: MeOH=10:1) to give tert-butyl 4-[2-[3-[2-(6-methyl-7-oxo-1H-pyrrolo[2,3-c]pyridin-4-yl)-4-(propanoylamino)phenoxy]phenoxy]ethoxy]piperidine-1-carboxylate (30 mg, 55% yield) as a yellow oil.

### 1.2 Synthesis of Compound 3

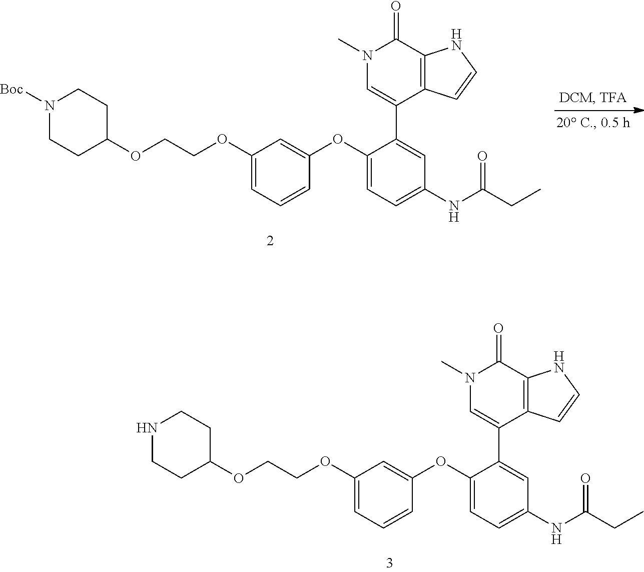 [SMILES: CCC(=O)Nc1ccc(Oc2cccc(OCCOC3CCN(C(=O)OC(C)(C)C)CC3)c2)c(-c2cn(C)c(=O)c3[nH]ccc23)c1 ; CCC(=O)Nc1ccc(Oc2cccc(OCCOC3CCNCC3)c2)c(-c2cn(C)c(=O)c3[nH]ccc23)c1]

To a solution of tert-butyl 4-[2-[3-[2-(6-methyl-7-oxo-1H-pyrrolo[2,3-c]pyridin-4-yl)-4-(propanoylamino)phenoxy]phenoxy]ethoxy]piperidine-1-carboxylate (30 mg, 47 mol, 1.0 equiv) in DCM (1.5 mL) and TFA (0.5 mL). The mixture was stirred at 20° C. for 0.5 h. The solution was concentrated under reduced pressure to give N-[3-(6-methyl-7-oxo-1H-pyrrolo[2,3-c]pyridin-4-yl)-4-[3-[2-(4-piperidyloxy)ethoxy]phenoxy]phenyl]propanamide (30 mg, crude, TFA salt) as a yellow oil.

### 1.3 Synthesis of I-59

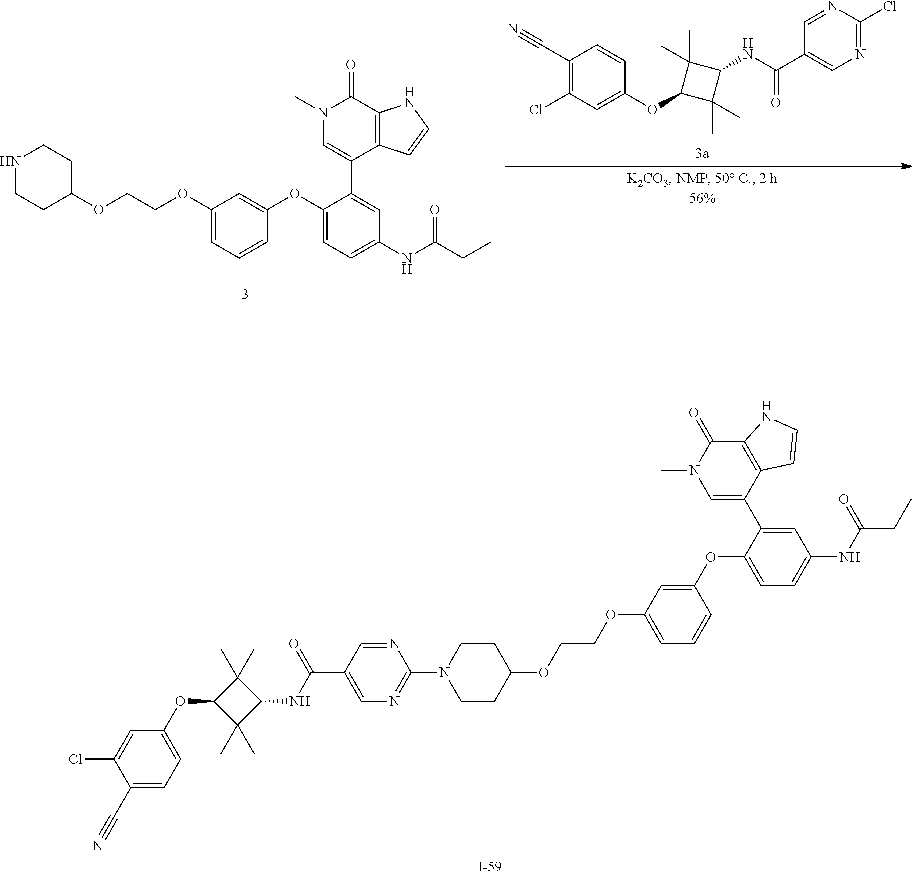

To a solution of N-[3-(6-methyl-7-oxo-1H-pyrrolo[2,3-c]pyridin-4-yl)-4-[3-[2-(4-piperidyloxy)ethoxy]phenoxy]phenyl]propanamide (30 mg, 46 mol, 1.0 equiv, TFA) in NMP (1 mL) was added K₂CO₃ (20 mg, 140 μmol, 3.0 equiv) and 2-chloro-N-[3-(3-chloro-4-cyano-phenoxy)-2,2,4,4-tetramethyl-cyclobutyl]pyrimidine-5-carboxamide (19 mg, 46 μmol, 1.0 equiv). The mixture was stirred at 50° C. for 2 h and concentrated. The residue was purified by prep-HPLC (column: Waters Xbridge 150 mm*25 mm*5 μm; mobile phase: [water(NH₄HCO₃)-ACN]; B %: 53%-83%, 9 min) to give N-[3-(3-chloro-4-cyano-phenoxy)-2,2,4,4-tetramethyl-cyclobutyl]-2-[4-[2-[3-[2-(6-methyl-7-oxo-1H-pyrrolo[2,3-c]pyridin-4-yl)-4-(propanoylamino)phenoxy]phenoxy]ethoxy]-1-piperidyl]pyrimidine-5-carboxamide (24 mg, 56% yield) as a white solid. ¹H NMR (400 MHz, DMSO-d₆): δ 12.06-11.98 (m, 1H), 9.97 (s, 1H), 8.75 (s, 2H), 7.90 (d, J=8.8 Hz, 1H), 7.84 (s, 1H), 7.74-7.68 (m, 1H), 7.61-7.55 (m, 1H), 7.30-7.24 (m, 2H), 7.23-7.19 (m, 1H), 7.15-7.08 (m, 1H), 7.06-6.98 (m, 2H), 6.58-6.52 (m, 1H), 6.41-6.33 (m, 2H), 6.32-6.28 (m, 1H), 4.29 (s, 1H), 4.27-4.18 (m, 2H), 4.06-4.01 (m, 1H), 4.00-3.94 (m, 2H), 3.74-3.69 (m, 2H), 3.68-3.60 (m, 2H), 3.48 (s, 4H), 2.36-2.28 (m, 3H), 1.91-1.82 (m, 2H), 1.47-1.36 (m, 2H), 1.21 (s, 6H), 1.11 (s, 6H), 1.09-1.06 (m, 2H).

### Example 13—Synthesis of N-((1r, 3r)-3-(3-chloro-4-cyanophenoxy)-2,2,4,4-tetramethylcyclobutyl)-2-(4-(2-(3-(2-(6-methyl-7-oxo-6,7-dihydro-1H-pyrrolo[2,3-c]pyridin-4-yl)phenoxy)phenoxy)ethoxy)piperidin-1-yl)pyrimidine-5-carboxamide (I-61)

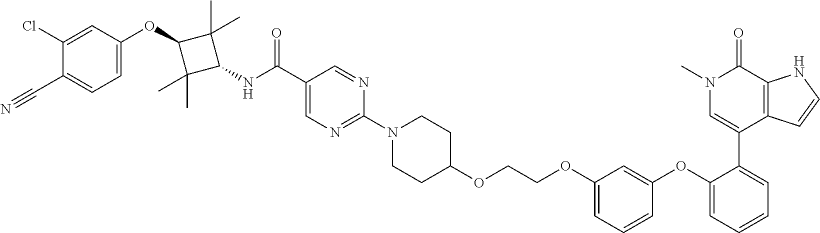 [SMILES: Cn1cc(-c2ccccc2Oc2cccc(OCCOC3CCN(c4ncc(C(=O)N[C@H]5C(C)(C)[C@H](Oc6ccc(C#N)c(Cl)c6)C5(C)C)cn4)CC3)c2)c2cc[nH]c2c1=O]

### 1.1 Synthesis of Compound 2

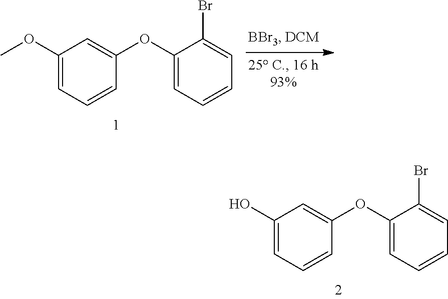 [SMILES: COc1cccc(Oc2ccccc2Br)c1 ; Oc1cccc(Oc2ccccc2Br)c1]

To a solution of 1-bromo-2-(3-methoxyphenoxy)benzene (2 g, 7.17 mol, 1.0 equiv) in DCM (60 mL) was added BBr₃ (5.39 g, 21.5 μmol, 3.0 equiv) at 0° C. The mixture was stirred at 25° C. for 16 h. The mixture was quenched by slowly adding MeOH (30 mL) at 0° C. and stirred at 25° C. for 0.5 h. The mixture was concentrated. The residue was purified by column chromatography (SiO₂, Petroleum ether/Ethyl acetate=50/1 to 4/1) to give 3-(2-bromophenoxy)phenol (1.78 g, 93% yield).

### 1.2 Synthesis of Compound 4

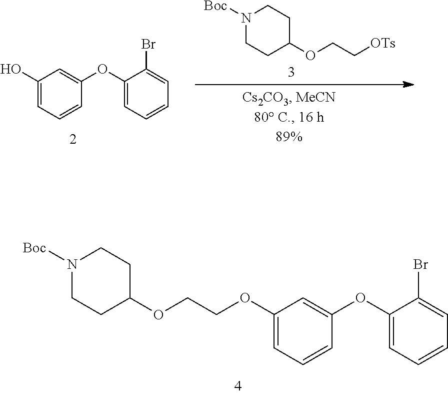 [SMILES: Oc1cccc(Oc2ccccc2Br)c1 ; Cc1ccc(S(=O)(=O)OCCOC2CCN(C(=O)OC(C)(C)C)CC2)cc1 ; CC(C)(C)OC(=O)N1CCC(OCCOc2cccc(Oc3ccccc3Br)c2)CC1]

To a solution of tert-butyl 4-[2-(p-tolylsulfonyloxy)ethoxy]piperidine-1-carboxylate (1.50 g, 3.75 μmol, 1.0 equiv) and 3-(2-bromophenoxy)phenol (995 mg, 3.75 μmol, 1.0 equiv) in MeCN (15 mL) was added Cs₂CO₃ (3.67 g, 11.2 mol, 3.0 equiv). The mixture was stirred at 80° C. for 16 h and concentrated. The residue was purified by column chromatography (SiO₂, Petroleum ether/Ethyl acetate=30/1 to 3/1) to give tert-butyl 4-[2-[3-(2-bromophenoxy) phenoxy]ethoxy]piperidine-1-carboxylate (1.75 g, 89% yield).

### 1.3 Synthesis of 6

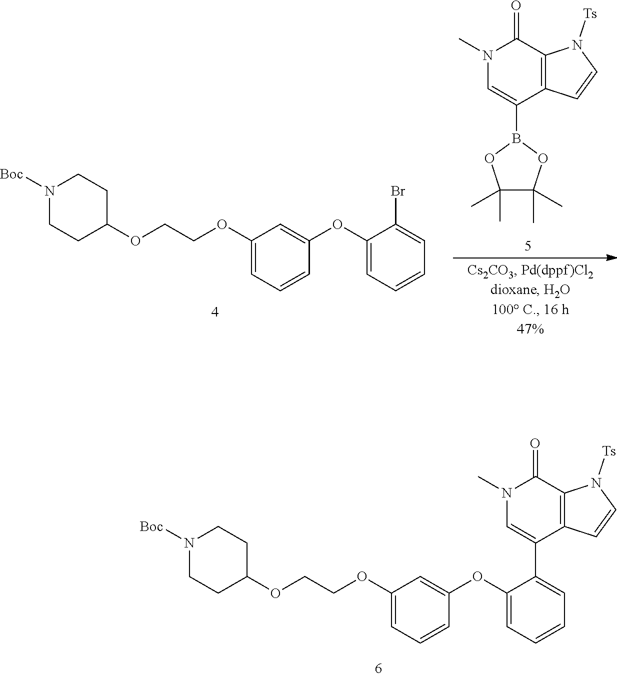 [SMILES: CC(C)(C)OC(=O)N1CCC(OCCOc2cccc(Oc3ccccc3Br)c2)CC1 ; Cc1ccc(S(=O)(=O)n2ccc3c(B4OC(C)(C)C(C)(C)O4)cn(C)c(=O)c32)cc1 ; Cc1ccc(S(=O)(=O)n2ccc3c(-c4ccccc4Oc4cccc(OCCOC5CCN(C(=O)OC(C)(C)C)CC5)c4)cn(C)c(=O)c32)cc1]

To a solution of tert-butyl 4-[2-[3-(2-bromophenoxy)phenoxy]ethoxy]piperidine-1-carboxylate (160 mg, 324 μmol, 1.0 equiv), 6-methyl-1-(p-tolylsulfonyl)-4-(4,4,5,5-tetramethyl-1,3,2-dioxaborolan-2-yl)pyrrolo[2,3-c]pyridin-7-one (167 mg, 389 μmol, 1.2 equiv) and Cs₂CO₃ (317 mg, 974 μmol, 3.0 equiv) in dioxane (5 mL) and H₂O (1 mL) was added Pd(dppf)Cl₂ (47 mg, 64 μmol, 0.2 equiv). The mixture was stirred at 100° C. for 16 h under N₂. The mixture was filtered and concentrated. The residue was purified by prep-TLC (SiO₂, Petroleum ether/Ethyl acetate=0:1) to give tert-butyl 4-[2-[3-[2-[6-methyl-7-oxo-1-(p-tolylsulfonyl)pyrrolo[2,3-c]pyridin-4-yl]phenoxy]phenoxy]ethoxy]piperidine-1-carboxylate (120 mg, 47% yield).

### 1.4 Synthesis of 7

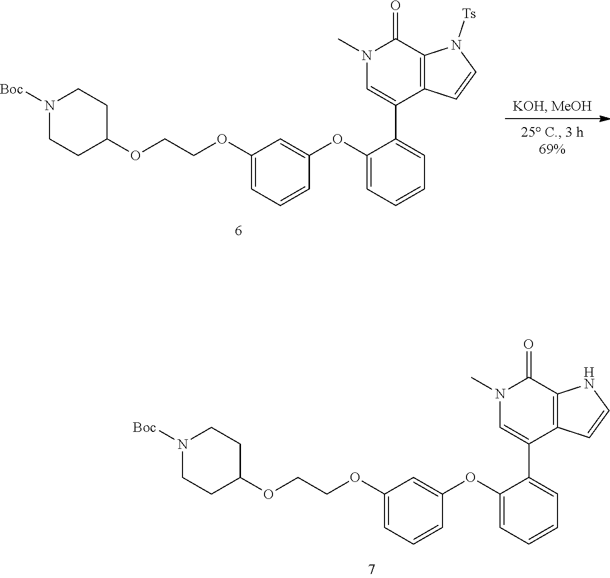 [SMILES: Cc1ccc(S(=O)(=O)n2ccc3c(-c4ccccc4Oc4cccc(OCCOC5CCN(C(=O)OC(C)(C)C)CC5)c4)cn(C)c(=O)c32)cc1 ; Cn1cc(-c2ccccc2Oc2cccc(OCCOC3CCN(C(=O)OC(C)(C)C)CC3)c2)c2cc[nH]c2c1=O]

To a solution of tert-butyl 4-[2-[3-[2-[6-methyl-7-oxo-1-(p-tolylsulfonyl) pyrrolo[2,3-c]pyridin-4-yl]phenoxy]phenoxy]ethoxy]piperidine-1-carboxylate (110 mg, 154 μmol, 1.0 equiv) in MeOH (2 mL) was added KOH (26 mg, 462 mol, 3.0 equiv), the mixture was stirred at 25° C. for 3 h. The mixture was filtered and concentrated The residue was purified by prep-TLC (SiO₂, Ethyl acetate: MeOH=10:1) to give tert-butyl 4-[2-[3-[2-(6-methyl-7-oxo-1H-pyrrolo[2,3-c]pyridin-4-yl)phenoxy]phenoxy]ethoxy]piperidine-1-carboxylate (60 mg, 69% yield).

### 1.5 Synthesis of 8

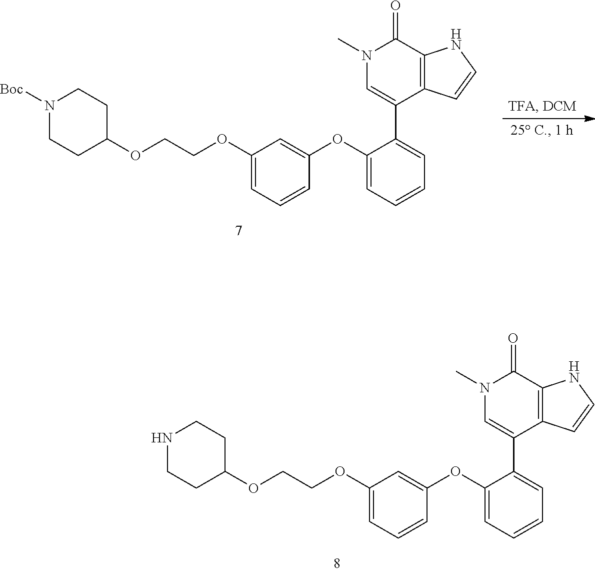 [SMILES: Cn1cc(-c2ccccc2Oc2cccc(OCCOC3CCN(C(=O)OC(C)(C)C)CC3)c2)c2cc[nH]c2c1=O ; Cn1cc(-c2ccccc2Oc2cccc(OCCOC3CCNCC3)c2)c2cc[nH]c2c1=O]

To a solution of tert-butyl 4-[2-[3-[2-(6-methyl-7-oxo-1H-pyrrolo[2,3-c]pyridin-4-yl)phenoxy]phenoxy]ethoxy]piperidine-1-carboxylate (60 mg, 107 gmol, 1.0 equiv) in DCM (1.5 mL) was added TFA (500 L). The mixture was stirred at 25° C. for 1 h. The mixture was concentrated to give crude product 6-methyl-4-[2-[3-[2-(4-piperidyloxy)ethoxy]phenoxy]phenyl]-1H-pyrrolo[2,3-c]pyridin-7-one (61 mg, 106 mol, TFA salt) as a yellow gum.

### 1.6 Synthesis of I-61

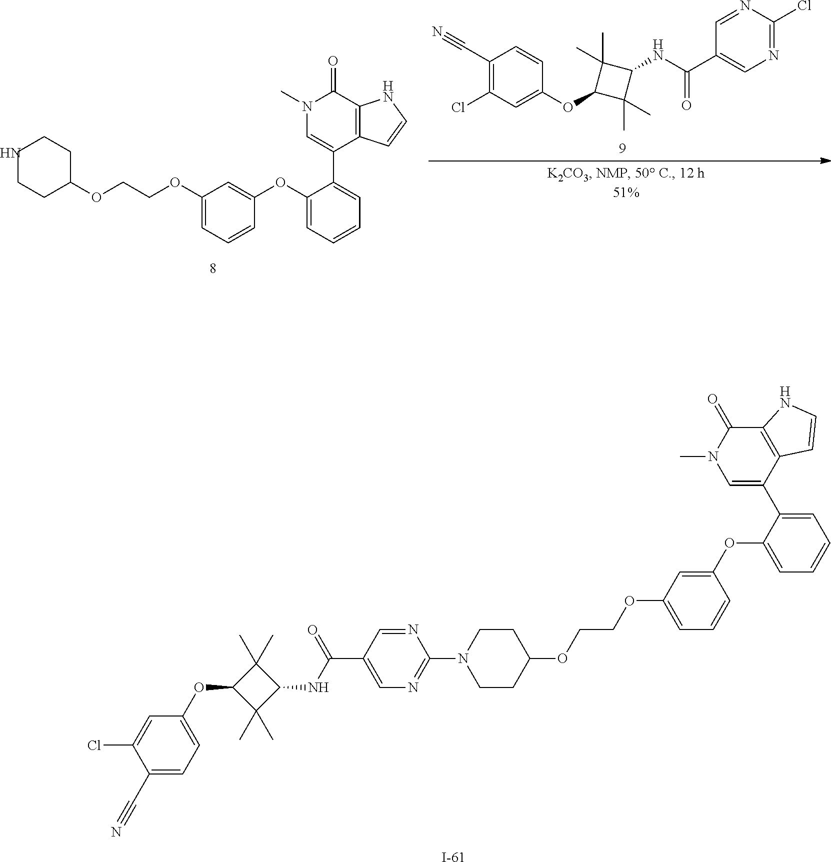

To a solution of 6-methyl-4-[2-[3-[2-(4-piperidyloxy)ethoxy]phenoxy]phenyl]-1H-pyrrolo[2,3-c]pyridin-7-one (61 mg, 106 μmol, 1.0 equiv, TFA salt) and 2-chloro-N-[3-(3-chloro-4-cyano-phenoxy)-2,2,4,4-tetramethyl-cyclobutyl]pyrimidine-5-carboxamide (49 mg, 116 μmol, 1.1 equiv) in NMP (1 mL) was added K₂CO₃ (73 mg, 531 mol, 5.0 equiv), the mixture was stirred at 50° C. for 12 h. The mixture was filtered and concentrated. The residue was purified by prep-HPLC (column: Waters Xbridge 150 mm*25 mm*5 μm; mobile phase: [water(NH₄HCO₃)-ACN]; B %: 60%-90%, 9 min) to give N-[3-(3-chloro-4-cyano-phenoxy)-2,2,4,4-tetramethyl-cyclobutyl]-2-[4-[2-[3-[2-(6-methyl-7-oxo-1H-pyrrolo[2,3-c]pyridin-4-yl)phenoxy]phenoxy]ethoxy]-1-piperidyl]pyrimidine-5-carboxamide (47 mg, 51% yield). 1H NMR (400 MHz, DMSO-d₆): δ 12.0 (s, 1H), 8.76 (s, 2H), 7.90 (d, J=8.8 Hz, 1H), 7.72 (d, J=9.2 Hz, 1H), 7.50 (d, J=7.6 Hz, 1H), 7.42-7.34 (m, 1H), 7.29-7.20 (m, 4H), 7.19-7.12 (m, 1H), 7.06-6.95 (m, 2H), 6.66-6.58 (m, 1H), 6.48-6.40 (m, 2H), 6.22 (s, 1H), 4.29 (s, 1H), 4.27-4.18 (m, 2H), 4.07-3.97 (m, 3H), 3.77-3.70 (m, 2H), 3.69-3.59 (m, 1H), 3.50 (s, 3H), 3.49-3.42 (m, 2H), 1.92-1.81 (m, 2H), 1.48-1.37 (m, 2H), 1.21 (s, 6H), 1.11 (s, 6H).

### Example 14—Synthesis of 2-(4-(2-(3-(4-amino-2-(6-methyl-7-oxo-6,7-dihydro-1H-pyrrolo[2,3-c]pyridin-4-yl)phenoxy)phenoxy)ethoxy)piperidin-1-yl)-N-((1r, 3r)-3-(3-chloro-4-cyanophenoxy)-2,2,4,4-tetramethylcyclobutyl)pyrimidine-5-carboxamide (I-62)

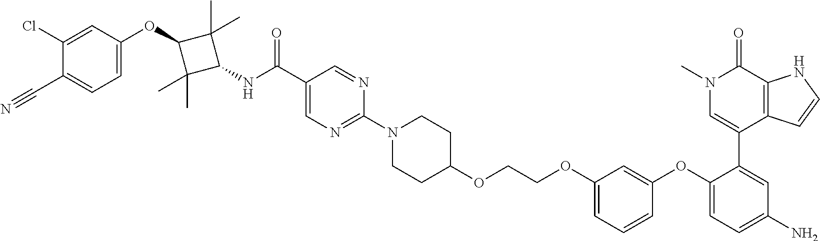 [SMILES: Cn1cc(-c2cc(N)ccc2Oc2cccc(OCCOC3CCN(c4ncc(C(=O)N[C@H]5C(C)(C)[C@H](Oc6ccc(C#N)c(Cl)c6)C5(C)C)cn4)CC3)c2)c2cc[nH]c2c1=O]

### 1.1 Synthesis of Compound 2

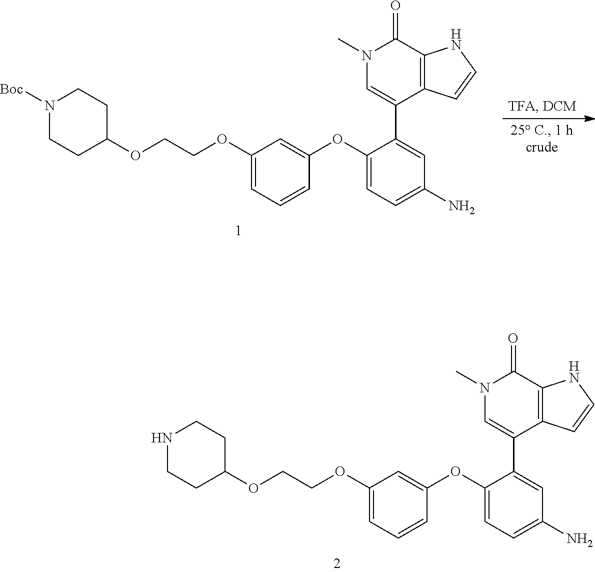 [SMILES: Cn1cc(-c2cc(N)ccc2Oc2cccc(OCCOC3CCN(C(=O)OC(C)(C)C)CC3)c2)c2cc[nH]c2c1=O ; Cn1cc(-c2cc(N)ccc2Oc2cccc(OCCOC3CCNCC3)c2)c2cc[nH]c2c1=O]

To a solution of tert-butyl 4-[2-[3-[4-amino-2-(6-methyl-7-oxo-1H-pyrrolo[2,3-c]pyridin-4-yl)phenoxy]phenoxy]ethoxy]piperidine-1-carboxylate (40 mg, 70 mol, 1.0 equiv) in DCM (1 mL) was added TFA (0.2 mL). The mixture was stirred at 25° C. for 1 h and concentrated under reduced pressure to give a crude product.

### 1.2 Synthesis of I-62

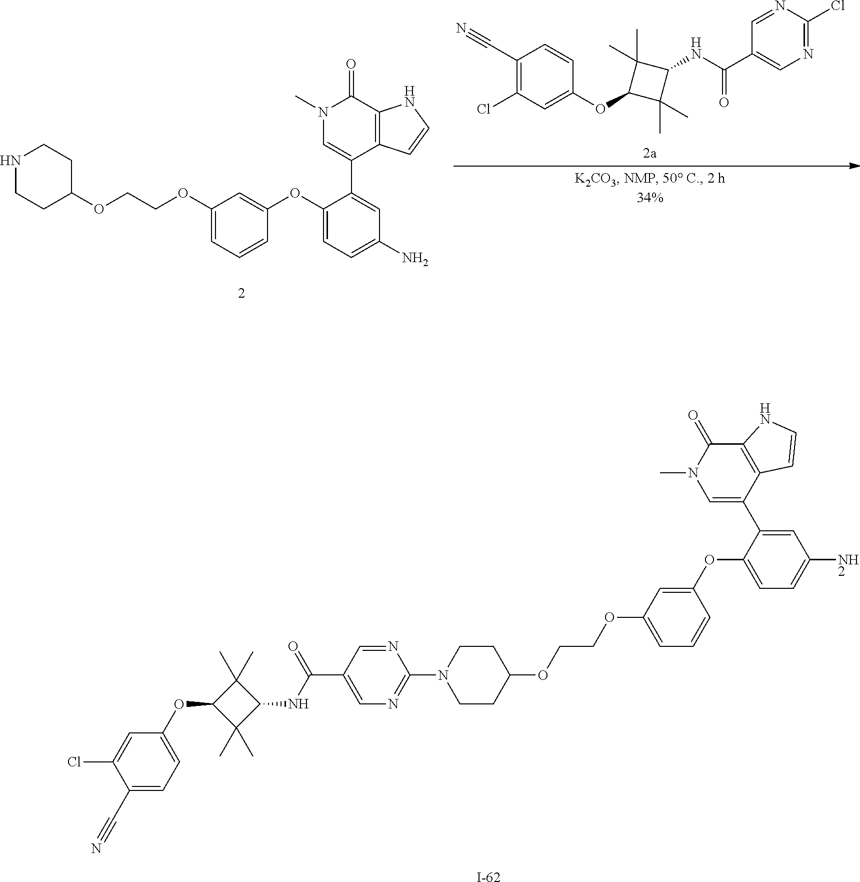

To a solution of 4-[5-amino-2-[3-[2-(4-piperidyloxy)ethoxy]phenoxy]phenyl]-6-methyl-1H-pyrrolo[2,3-c]pyridin-7-one (41 mg, 70 mol, 1.0 equiv, TFA salt) in NMP (1 mL) was added K₂CO₃ (29 mg, 208 μmol, 3.0 equiv) and 2-chloro-N-[3-(3-chloro-4-cyano-phenoxy)-2,2,4,4-tetramethyl-cyclobutyl]pyrimidine-5-carboxanide (29 mg, 70 mol, 1.0 equiv). The mixture was stirred at 50° C. for 2 h and concentrated. The residue was purified by prep-HPLC (column: Waters Xbridge 150 mm*25 mm*5 μm; mobile phase: [water(NH₄HCO₃)-ACN]; B %: 50%-80%, 9 min) to give 2-[4-[2-[3-[4-amino-2-(6-methyl-7-oxo-1H-pyrrolo[2,3-c]pyridin-4-yl)phenoxy]phenoxy]ethoxy]-1-piperidyl]-N-[3-(3-chloro-4-cyano-phenoxy)-2,2,4,4-tetramethyl-cyclobutyl]pyrimidine-5-carboxamide (21 mg, 34% yield) as a white solid. ¹H NMR (400 MHz, DMSO-d₆): 11.95 (s, 1H), 8.76 (s, 2H), 7.90 (d, J=8.6 Hz, 1H), 7.72 (d, J=9.0 Hz, 1H), 7.24 (t, J=2.4 Hz, 1H), 7.21 (d, J=2.4 Hz, 1H), 7.14 (s, 1H), 6.97-7.09 (m, 3H), 6.83 (d, J=8.6 Hz, 1H), 6.75 (d, J=2.6 Hz, 1H), 6.59 (m, 1H), 6.45 (m, 1H), 6.21-6.30 (m, 4H), 5.06-5.12 (m, 2H), 4.29 (s, 1H), 4.20-4.25 (m, 2H), 4.04 (d, J=9.0 Hz, 1H), 3.91-3.94 (m, 2H), 3.68-3.72 (m, 2H), 3.61-3.65 (m, 1H), 3.44 (s, 3H), 1.84-1.89 (m, 2H), 1.37-1.44 (m, 2H), 1.21 (s, 6H), 1.11 (s, 6H).

### Example 15—Synthesis of N-((1r, 3r)-3-(3-chloro-4-cyanophenoxy)-2,2,4,4-tetramethylcyclobutyl)-2-(4-(2-(3-(2-(6-methyl-7-oxo-6,7-dihydro-1H-pyrrolo[2,3-c]pyridin-4-yl)-4-nitrophenoxy)phenoxy)ethoxy)piperidin-1-yl)pyrimidine-5-carboxamide (I-63)

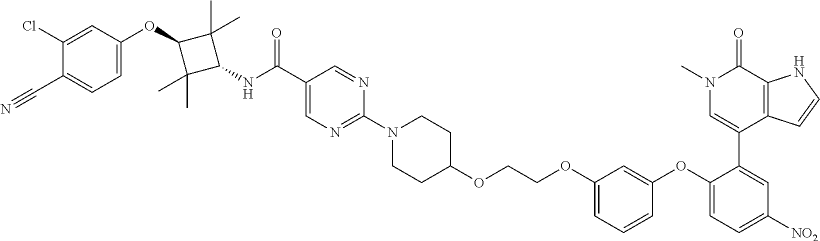 [SMILES: Cn1cc(-c2cc([N+](=O)[O-])ccc2Oc2cccc(OCCOC3CCN(c4ncc(C(=O)N[C@H]5C(C)(C)[C@H](Oc6ccc(C#N)c(Cl)c6)C5(C)C)cn4)CC3)c2)c2cc[nH]c2c1=O]

### 1.1 Synthesis of Compound 2

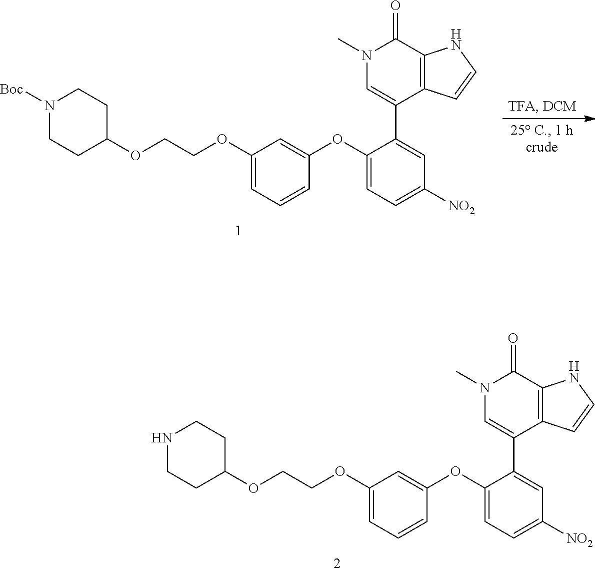 [SMILES: Cn1cc(-c2cc([N+](=O)[O-])ccc2Oc2cccc(OCCOC3CCN(C(=O)OC(C)(C)C)CC3)c2)c2cc[nH]c2c1=O ; Cn1cc(-c2cc([N+](=O)[O-])ccc2Oc2cccc(OCCOC3CCNCC3)c2)c2cc[nH]c2c1=O]

To a solution of tert-butyl 4-[2-[3-[2-(6-methyl-7-oxo-1H-pyrrolo[2,3-c]pyridin-4-yl)-4-nitro-phenoxy]phenoxy]ethoxy]piperidine-1-carboxylate (40 mg, 66 mol, 1.0 equiv) in DCM (1 mL) was added TFA (0.2 mL). The mixture was stirred at 25° C. for 1 h and concentrated under reduced pressure to give a crude product.

### 1.2 Synthesis of I-63

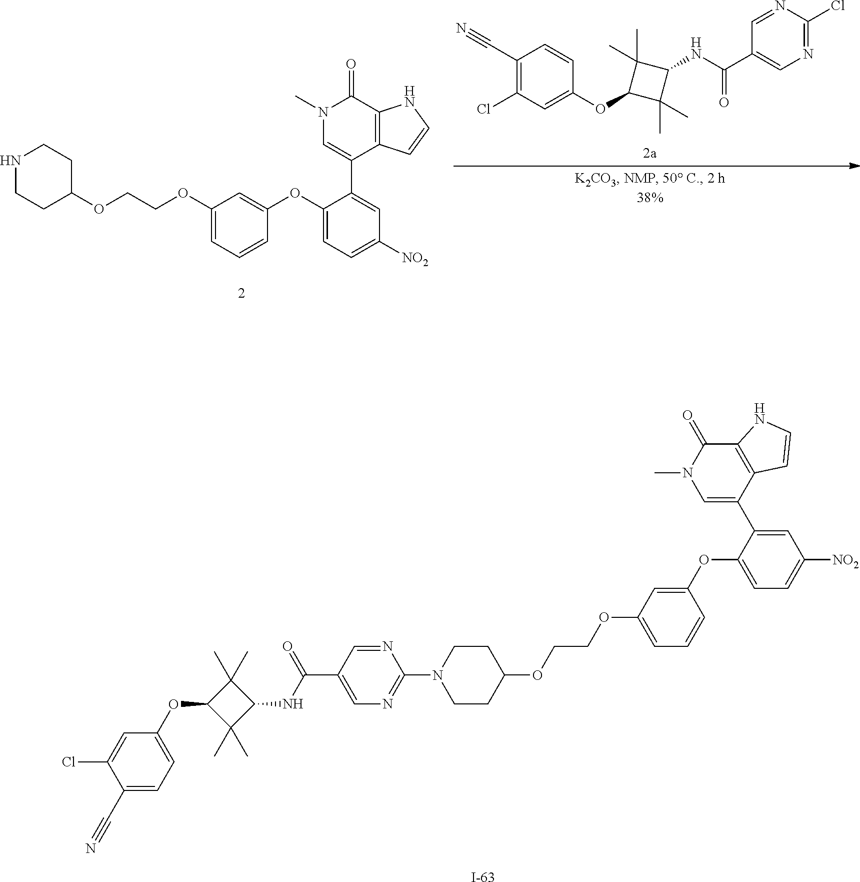

To a solution of 6-methyl-4-[5-nitro-2-[3-[2-(4-piperidyloxy)ethoxy]phenoxy]phenyl]-1H-pyrrolo[2,3-c]pyridin-7-one (41 mg, 66 μmol, 1.0 equiv, TFA salt) in NMP (1 mL) was added K₂CO₃ (27 mg, 198 mol, 3 equiv) and 2-chloro-N-[3-(3-chloro-4-cyano-phenoxy)-2,2,4,4-tetramethyl-cyclobutyl]pyrimidine-5-carboxamide (30 mg, 73 mol, 1.0 equiv). The mixture was stirred at 50° C. for 2 h and concentrated. The residue was purified by prep-HPLC (column: Waters Xbridge 150 mm*25 mm*5 μm; mobile phase: [water(NH₄HCO₃)-ACN]; B %: 57%-87%, 9 min) to give N-[3-(3-chloro-4-cyano-phenoxy)-2,2,4,4-tetramethyl-cyclobutyl]-2-[4-[2-[3-[2-(6-methyl-7-oxo-1H-pyrrolo[2,3-c]pyridin-4-yl)-4-nitro-phenoxy]phenoxy]ethoxy]-1-piperidyl]pyrimidine-5-carboxamide (22 mg, 38% yield) as a yellow solid. ¹H NMR (400 MHz, DMSO-d₆): 12.10 (s, 1H), 8.75 (s, 2H), 8.31 (d, J=2.8 Hz, 1H), 8.22 (m, 1H), 7.90 (d, J=8.6 Hz, 1H), 7.71 (d, J=9.2 Hz, 1H), 7.47 (s, 1H), 7.29-7.36 (m, 2H), 7.21 (d, J=2.4 Hz, 1H), 6.99-7.04 (m, 2H), 6.84 (m, 1H), 6.77 (t, J=2.2 Hz, 1H), 6.73 (m, 1H), 6.32 (s, 1H), 4.29 (s, 1H), 4.20-4.25 (m, 2H), 4.08-4.11 (m, 2H), 4.03 (d, J=9.0 Hz, 1H), 3.76-3.79 (m, 2H), 3.65-3.69 (m, 1H), 3.57 (s, 3H), 3.44-3.50 (m, 2H), 1.86-1.91 (m, 2H), 1.43 (m, 2H), 1.21 (s, 6H), 1.11 (s, 6H).

### Example 16—Synthesis of N-((1r, 3r)-3-(3-chloro-4-cyanophenoxy)-2,2,4,4-tetramethylcyclobutyl)-2-(4-(2-(3-(4-(ethylsulfonamido)-2-(6-methyl-7-oxo-6,7-dihydro-1H-pyrrolo[2,3-c]pyridin-4-yl)phenoxy)-N-methylbenzamido)ethoxy)piperidin-1-yl)pyrimidine-5-carboxamide (I-66)

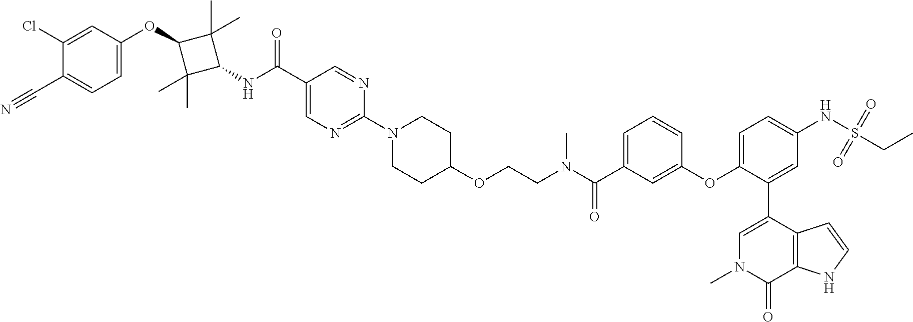 [SMILES: CCS(=O)(=O)Nc1ccc(Oc2cccc(C(=O)N(C)CCOC3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1]

### 1.1 Synthesis of Compound 2

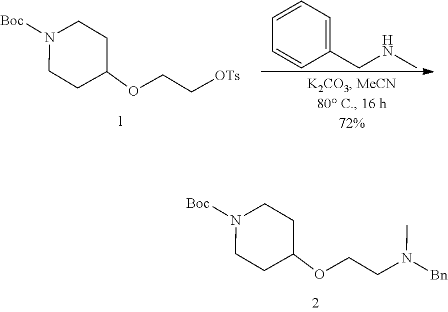

To a solution of tert-butyl 4-[2-(p-tolylsulfonyloxy)ethoxy]piperidine-1-carboxylate (1.5 g, 3.75 μmol, 1.0 equiv) and N-methyl-1-phenyl-methanamine (454 mg, 3.75 mol, 1.0 equiv) in MeCN (20 mL) was added K₂CO₃ (1.0 g, 7.5 gmol, 2.0 equiv). The mixture was stirred at 80° C. for 16 h and concentrated. The residue was purified by prep-HPLC (column: Waters Xbridge C18 150 mm*50 mm*10 μm; mobile phase: [water(NH₄HCO₃)-ACN]; B %: 48%-78%, 10 min) to give tert-butyl 4-[2-[benzyl(methyl)amino]ethoxy]piperidine-1-carboxylate (950 mg, 72% yield) as a colorless oil. ¹H NMR (400 MHz, CDCl₃): δ 7.37-7.28 (m, 4H), 7.27-7.22 (m, 1H), 3.83-3.70 (m, 2H), 3.64-3.54 (m, 4H), 3.49-3.38 (m, 1H), 3.17-2.98 (m, 2H), 2.62 (t, J=6.0 Hz, 2H), 2.28 (s, 3H), 1.87-1.76 (m, 2H), 1.54-1.44 (m, 11H).

### 1.2 Synthesis of Compound 3

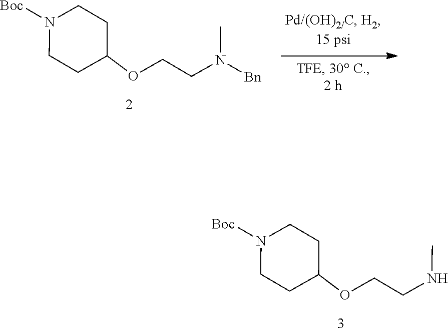 [SMILES: CN(CCOC1CCN(C(=O)OC(C)(C)C)CC1)Cc1ccccc1 ; CNCCOC1CCN(C(=O)OC(C)(C)C)CC1]

To a solution of tert-butyl 4-[2-[benzyl(methyl)amino]ethoxy]piperidine-1-carboxylate (950 mg, 2.73 gmol, 1.0 equiv) in TFE (10 mL) was added Pd(OH)₂/C (100 mg, 10% purity) under N₂. The mixture was stirred under H₂ (15 psi) at 30° C. for 2 h. The mixture was filtered and concentrated to give crude tert-butyl 4-[2-(methylamino)ethoxy]piperidine-1-carboxylate (700 mg) as a colorless oil. ¹H NMR (400 MHz, CDCl₃): δ 3.95-3.83 (m, 1H), 3.81-3.68 (m, 2H), 3.58 (t, J=4.8 Hz, 2H), 3.49-3.40 (m, 1H), 3.16-2.99 (m, 2H), 2.75 (t, J=5.2 Hz, 2H), 2.46 (s, 3H), 1.91-1.72 (m, 2H), 1.61-1.35 (m, 11H).

### 1.3 Synthesis of 4

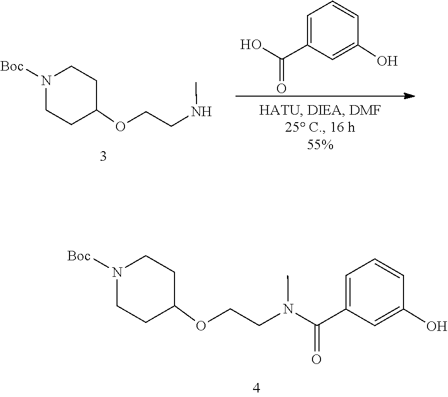 [SMILES: CNCCOC1CCN(C(=O)OC(C)(C)C)CC1 ; O=C(O)c1cccc(O)c1 ; CN(CCOC1CCN(C(=O)OC(C)(C)C)CC1)C(=O)c1cccc(O)c1]

To a solution of tert-butyl 4-[2-(methylamino)ethoxy]piperidine-1-carboxylate (650 mg, 2.5 μmol, 1.0 equiv) and 3-hydroxybenzoic acid (347 mg, 2.52 μmol, 1.0 equiv) in DMF (5 mL) was added DIEA (1.3 g, 10 μmol, 4.0 equiv) and HATU (1.15 g, 3 mol, 1.2 equiv). The mixture was stirred at 25° C. for 16 h and concentrated. The residue was purified by prep-HPLC (column: Phenomenex luna C18 150 mm*40 μm*15 μm; mobile phase: [water(FA)-ACN]; B %: 32%-62%, 10 min) to give tert-butyl 4-[2-[(3-hydroxybenzoyl)-methyl-amino]ethoxy]piperidine-1-carboxylate (530 mg, 55% yield) as a white solid. ¹H NMR (400 MHz, DMSO-d₆): δ 9.74-9.47 (m, 1H), 7.30-7.10 (m, 1H), 6.89-6.56 (m, 3H), 3.69-3.40 (m, 6H), 3.04 (s, 2H), 2.93 (s, 3H), 1.86-1.60 (m, 2H), 1.45-1.24 (m, 11H).

### 1.4 Synthesis of 6

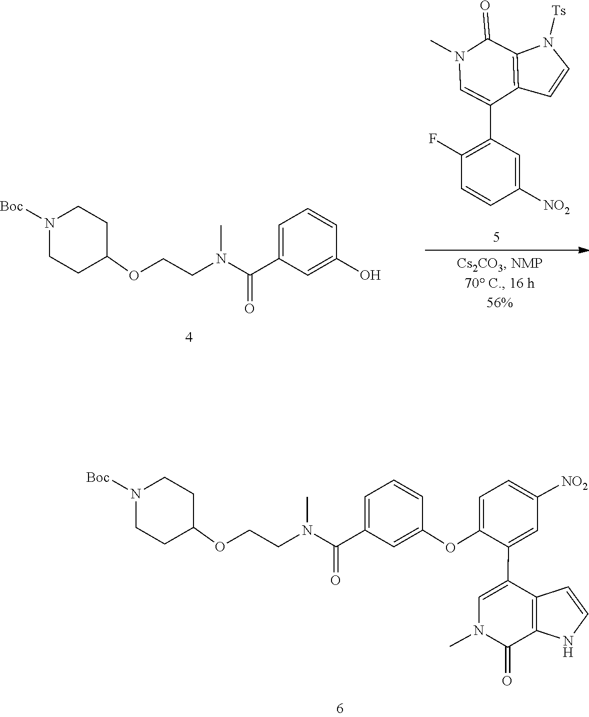 [SMILES: Cc1ccc(S(=O)(=O)n2ccc3c(-c4cc([N+](=O)[O-])ccc4F)cn(C)c(=O)c32)cc1 ; CN(CCOC1CCN(C(=O)OC(C)(C)C)CC1)C(=O)c1cccc(O)c1 ; CN(CCOC1CCN(C(=O)OC(C)(C)C)CC1)C(=O)c1cccc(Oc2ccc([N+](=O)[O-])cc2-c2cn(C)c(=O)c3[nH]ccc23)c1]

To a solution of tert-butyl 4-[2-[(3-hydroxybenzoyl)-methyl-amino]ethoxy]piperidine-1-carboxylate (180 mg, 475 μmol, 1.0 equiv) and 4-(2-fluoro-5-nitro-phenyl)-6-methyl-1-(p-tolylsulfonyl)pyrrolo[2,3-c]pyridin-7-one (209 mg, 475 μmol, 1.0 equiv) in NMP (3 mL) was added Cs₂CO₃ (464 mg, 1.43 mol, 3.0 equiv). The mixture was stirred at 70° C. for 16 h. The reaction mixture was diluted with water (30 mL) and extracted with EtOAc (3×30 mL). The combined organic layers were washed with brine (50 mL), dried over Na₂SO₄, filtered and concentrated. The residue was purified by prep-TLC (SiO₂, Ethyl acetate: Methanol=10:1) to give tert-butyl4-[2-[methyl-[3-[2-(6-methyl-7-oxo-1H-pyrrolo[2,3-c]pyridin-4-yl)-4-nitro-phenoxy]benzoyl]amino]ethoxy]piperidine-1-carboxylate (175 mg, 56% yield) as light yellow gum.

### 1.5 Synthesis of 7

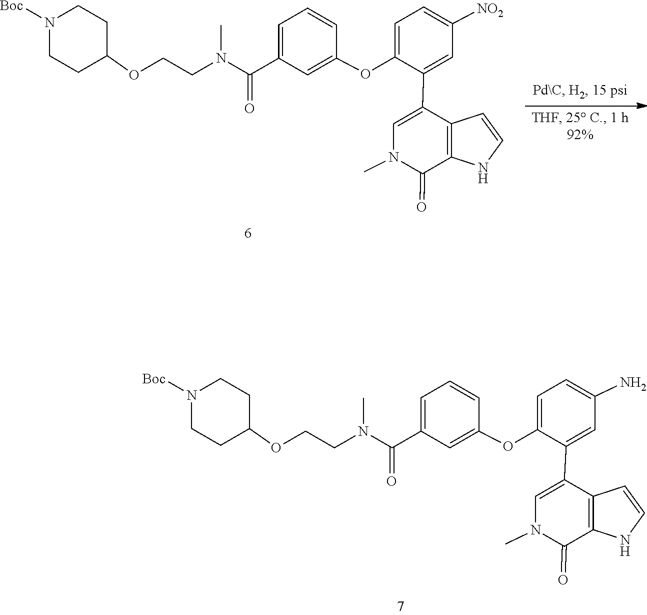 [SMILES: CN(CCOC1CCN(C(=O)OC(C)(C)C)CC1)C(=O)c1cccc(Oc2ccc([N+](=O)[O-])cc2-c2cn(C)c(=O)c3[nH]ccc23)c1 ; CN(CCOC1CCN(C(=O)OC(C)(C)C)CC1)C(=O)c1cccc(Oc2ccc(N)cc2-c2cn(C)c(=O)c3[nH]ccc23)c1]

To a solution of tert-butyl 4-[2-[methyl-[3-[2-(6-methyl-7-oxo-1H-pyrrolo[2,3-c]pyridin-4-yl)-4-nitro-phenoxy]benzoyl]amino]ethoxy]piperidine-1-carboxylate (170 mg, 263 μmol, 1.0 equiv) in THF (4 mL) was added Pd/C (30 mg, 10% purity) under N₂. The mixture was stirred under H₂ (15 psi) at 25° C. for 1 h. The mixture was filtered and concentrated to give tert-butyl 4-[2-[[3-[4-amino-2-(6-methyl-7-oxo-1H-pyrrolo[2,3-c]pyridin-4-yl)phenoxy]benzoyl]-methyl-amino]ethoxy]piperidine-1-carboxylate (150 mg, 92% yield) as a colorless gum.

### 1.6 Synthesis of 8

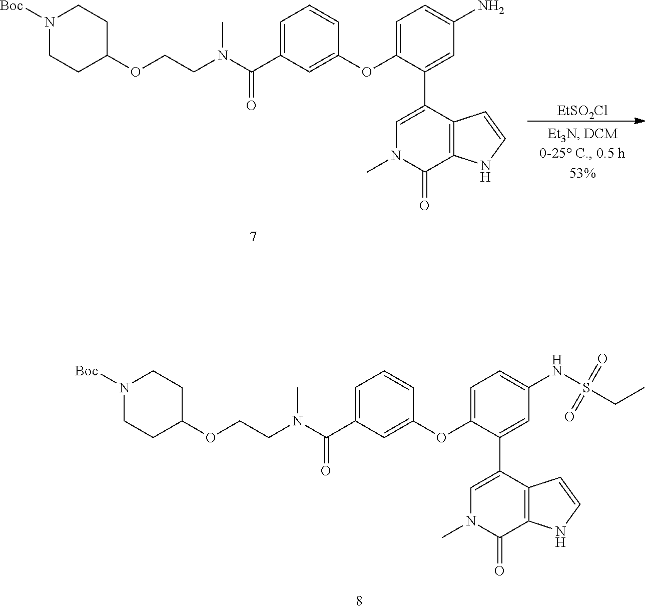 [SMILES: "EtSO2Cl" ; CN(CCOC1CCN(C(=O)OC(C)(C)C)CC1)C(=O)c1cccc(Oc2ccc(N)cc2-c2cn(C)c(=O)c3[nH]ccc23)c1 ; CCS(=O)(=O)Nc1ccc(Oc2cccc(C(=O)N(C)CCOC3CCN(C(=O)OC(C)(C)C)CC3)c2)c(-c2cn(C)c(=O)c3[nH]ccc23)c1]

To a solution of tert-butyl 4-[2-[[3-[4-amino-2-(6-methyl-7-oxo-1H-pyrrolo[2,3-c]pyridin-4-yl)phenoxy]benzoyl]-methyl-amino]ethoxy]piperidine-1-carboxylate (150 mg, 243 μmol, 1.0 equiv) and TEA (246 mg, 2.44 μmol, 10 equiv) in DCM (2 mL) was added ethanesulfonyl chloride (94 mg, 730 μmol, 3.0 equiv) at 0° C. The mixture was stirred at 25° C. for 0.5 h and concentrated. The residue was purified by prep-TLC (SiO₂, Ethyl acetate: Methanol=10:1) to give the desired product tert-butyl 4-[2-[[3-[4-(ethylsulfonylamino)-2-(6-methyl-7-oxo-1H-pyrrolo[2,3-c]pyridin-4-yl)phenoxy]benzoyl]-methyl-amino]ethoxy]piperidine-1-carboxylate (100 mg, 53% yield) as a colorless gum.

### 1.7 Synthesis of 9

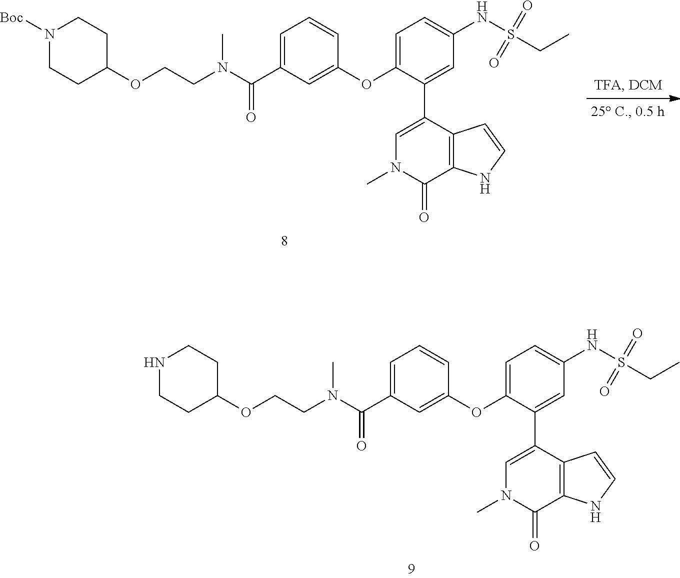 [SMILES: CCS(=O)(=O)Nc1ccc(Oc2cccc(C(=O)N(C)CCOC3CCN(C(=O)OC(C)(C)C)CC3)c2)c(-c2cn(C)c(=O)c3[nH]ccc23)c1 ; CCS(=O)(=O)Nc1ccc(Oc2cccc(C(=O)N(C)CCOC3CCNCC3)c2)c(-c2cn(C)c(=O)c3[nH]ccc23)c1]

To a solution of tert-butyl 4-[2-[[3-[4-(ethylsulfonylamino)-2-(6-methyl-7-oxo-1H-pyrrolo[2,3-c]pyridin-4-yl)phenoxy]benzoyl]-methyl-amino]ethoxy]piperidine-1-carboxylate (50 mg, 71 μmol, 1.0 equiv) in DCM (2 mL) was added TFA (1 mL). The mixture was stirred at 25° C. for 0.5 h and concentrated to give 3-[4-(ethylsulfonylamino)-2-(6-methyl-7-oxo-1H-pyrrolo[2,3-c]pyridin-4-yl)phenoxy]-N-methyl-N-[2-(4-piperidyloxy)ethyl]benzamide (50 mg, TFA salt) as a yellow gum.

### 1.8 Synthesis of I-66

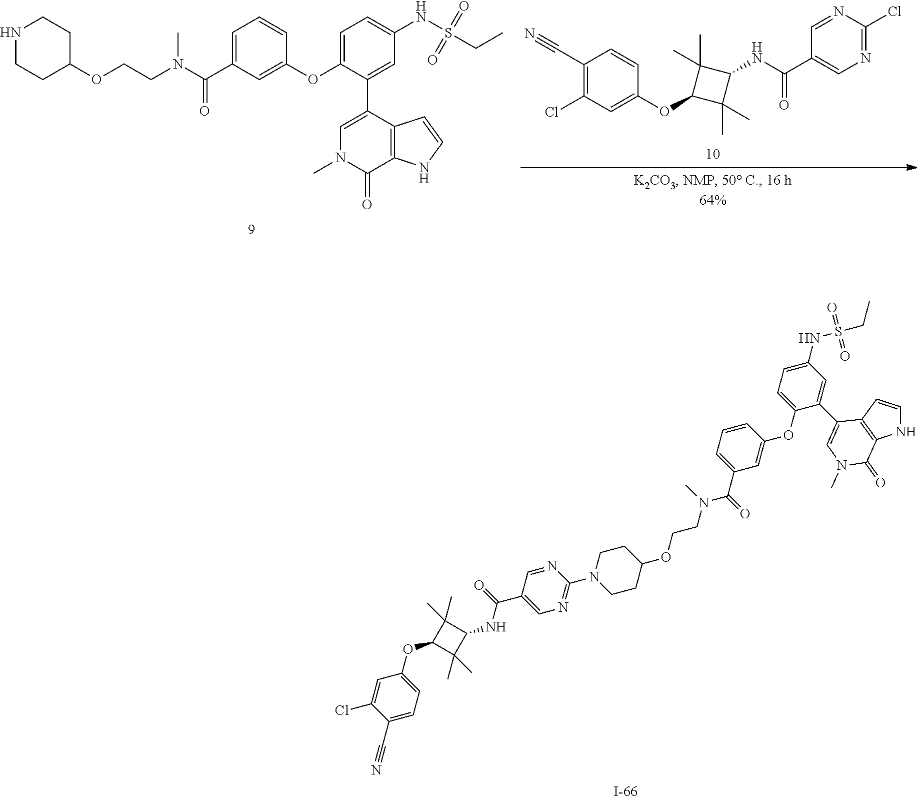

To a solution of 3-[4-(ethylsulfonylamino)-2-(6-methyl-7-oxo-1H-pyrrolo[2,3-c]pyridin-4-yl)phenoxy]-N-methyl-N-[2-(4-piperidyloxy)ethyl]benzamide (50 mg, 69 mol, 1.0 equiv, TFA salt) and 2-chloro-N-[3-(3-chloro-4-cyano-phenoxy)-2,2,4,4-tetramethyl-cyclobutyl]pyrimidine-5-carboxamide (32 mg, 76 μmol, 1.1 equiv) in NMP (1 mL) was added K₂CO₃ (48 mg, 346 mol, 5.0 equiv). The mixture was stirred at 50° C. for 16 h and concentrated. The residue was purified by prep-HPLC (column: Waters Xbridge 150 mm*25 mm*5 μm; mobile phase: [water(NH₄HCO₃)-ACN]; B %: 47%-77%, 9 min) to give N-[3-(3-chloro-4-cyano-phenoxy)-2,2,4,4-tetramethyl-cyclobutyl]-2-[4-[2-[[3-[4-(ethylsulfonylamino)-2-(6-methyl-7-oxo-1H-pyrrolo[2,3-c]pyridin-4-yl)phenoxy]benzoyl]-methyl-amino]ethoxy]-1-piperidyl]pyrimidine-5-carboxamide (44.8 mg, 64% yield) as a white solid. ¹H NMR (400 MHz, DMSO-d₆): δ 12.08-11.95 (m, 1H), 9.92-9.76 (m, 1H), 8.75 (s, 2H), 7.90 (d, J=8.8 Hz, 1H), 7.73-7.68 (m, 1H), 7.42-7.39 (m, 1H), 7.32-7.18 (m, 5H), 7.17-7.07 (m, 1H), 7.03-6.98 (m, 1H), 6.98-6.89 (m, 1H), 6.89-6.82 (m, 1H), 6.82-6.67 (m, 1H), 6.28-6.19 (m, 1H), 4.29 (s, 1H), 4.24-4.00 (m, 3H), 3.70-3.60 (m, 1H), 3.52-3.42 (m, 6H), 3.30 (s, 3H), 3.24-3.18 (m, 1H), 3.17-3.10 (m, 2H), 2.93-2.90 (m, 1H), 2.77-2.69 (m, 1H), 1.93-1.69 (m, 2H), 1.49-1.30 (m, 2H), 1.27-1.19 (m, 9H), 1.11 (s, 6H).

### Example 17—Synthesis of N-((1r, 3r)-3-(3-chloro-4-cyanophenoxy)-2,2,4,4-tetramethylcyclobutyl)-2-(4-(2-(1-cyclopropyl-2-(4-(ethylsulfonamido)-2-(6-methyl-7-oxo-6,7-dihydro-1H-pyrrolo[2,3-c]pyridin-4-yl)phenoxy)ethoxy)ethoxy)piperidin-1-yl)pyrimidine-5-carboxamide (I-68)

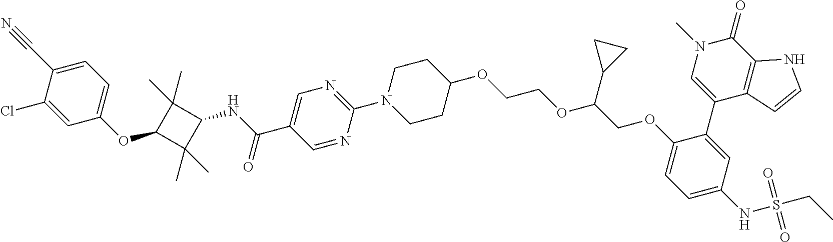 [SMILES: CCS(=O)(=O)Nc1ccc(OCC(OCCOC2CCN(c3ncc(C(=O)N[C@H]4C(C)(C)[C@H](Oc5ccc(C#N)c(Cl)c5)C4(C)C)cn3)CC2)C2CC2)c(-c2cn(C)c(=O)c3[nH]ccc23)c1]

### 1.1 Synthesis of Compound 2

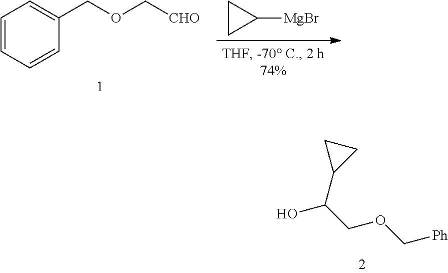

To a solution of 2-benzyloxyacetaldehyde (2 g, 13.3 mol, 1.0 equiv) in THF (20 mL) was added bromo (cyclopropyl) magnesium (0.5 M, 29.3 mL, 1.1 equiv) at −70° C. The mixture was stirred at −70° C. for 2 h under N₂ and quenched by addition 30 mL water. The mixture was extracted with EA (3×50 mL). The combined organic layers were washed with brine (100 mL), dried over anhydrous sodium sulfate, filtered and concentrated. The residue was purified by column chromatography (SiO₂, Petroleum ether/Ethyl acetate=10:1 to 1:1) to afford 2-benzyloxy-1-cyclopropyl-ethanol (1.9 g, 74% yield).

### 1.2 Synthesis of Compound 3

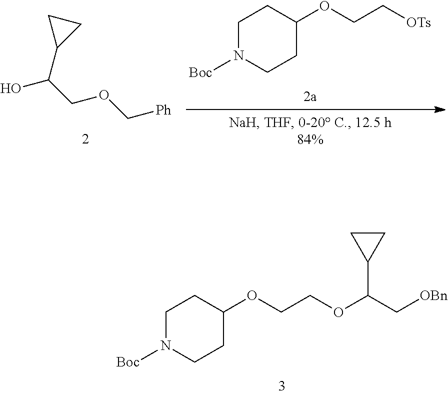 [SMILES: OC(COCc1ccccc1)C1CC1 ; Cc1ccc(S(=O)(=O)OCCOC2CCN(C(=O)OC(C)(C)C)CC2)cc1 ; CC(C)(C)OC(=O)N1CCC(OCCOC(COCc2ccccc2)C2CC2)CC1]

To a solution of 2-benzyloxy-1-cyclopropyl-ethanol (900 mg, 4.7 mol, 1.0 equiv) in THF (20 mL) was added dropwise NaH (280 mg, 7.0 μmol, 60% purity, 1.5 equiv) at 0° C. The mixture was stirred at this temperature for 0.5h and tert-butyl 4-[2-(p-tolylsulfonyloxy) ethoxy]piperidine-1-carboxylate (1.87 g, 4.7 μmol, 1.0 equiv) was added at 0° C. The mixture was stirred at 20° C. for 12 h and concentrated. The residue was diluted with H₂O (100 mL) and extracted with EA (3×50 mL). The combined organic layers were washed with H₂O (50 mL), dried over NA₂SO₄, filtered and concentrated. The residue was purified by flash silica gel chromatography (ISCO®; 20 g SepaFlash® Silica Flash Column, Eluent of 0˜30% Ethyl acetate/Petroleum ethergradient at 80 mE/min) to give tert-butyl 4-[2-(2-benzyloxy-1-cyclopropyl-ethoxy)ethoxy]piperidine-1-carboxylate (1.65 g, 84% yield).

### 1.3 Synthesis of Compound 4

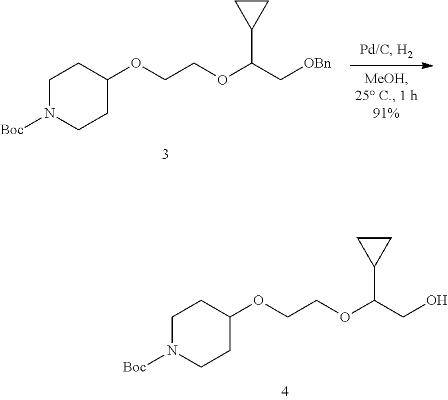 [SMILES: CC(C)(C)OC(=O)N1CCC(OCCOC(COCc2ccccc2)C2CC2)CC1 ; CC(C)(C)OC(=O)N1CCC(OCCOC(CO)C2CC2)CC1]

To a solution of tert-butyl 4-[2-(2-benzyloxy-1-cyclopropyl-ethoxy)ethoxy]piperidine-1-carboxylate (1.6 g, 3.8 mol, 1.0 equiv) and Pd/C (160 mg, 410 mol, 10% purity) in MeOH (50 mL). The mixture was stirred under H₂ (15 psi) at 20° C. for 12 h. The mixture was filtered and the filter was concentrated. The product was used directly for next step.

### 1.4 Synthesis of Compound 5

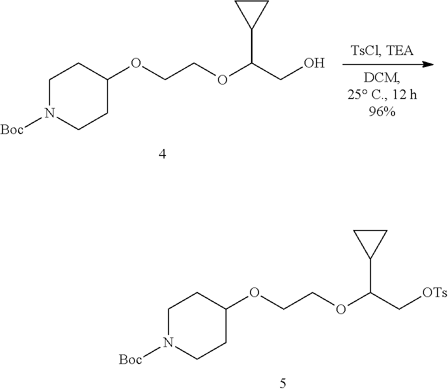 [SMILES: CC(C)(C)OC(=O)N1CCC(OCCOC(CO)C2CC2)CC1 ; Cc1ccc(S(=O)(=O)OCC(OCCOC2CCN(C(=O)OC(C)(C)C)CC2)C2CC2)cc1]

To a solution of tert-butyl 4-[2-(1-cyclopropyl-2-hydroxy-ethoxy)ethoxy]piperidine-1-carboxylate (1.14 g, 3.5 μmol, 1.0 equiv) in DCM (15 mL) was added TEA (1.0 g, 10.4 mol, 1.44 mL, 3.0 equiv) and TosCl (989 mg, 5.2 μmol, 1.5 equiv). The mixture was stirred at 25° C. for 12 h and concentrated. The residue was purified by flash silica gel chromatography (ISCO®; 10 g SepaFlash® Silica Flash Column, Eluent of 0˜25% Ethyl acetate/Petroleum ether gradient at 60 mL/min) to give tert-butyl 4-[2-[1-cyclopropyl-2-(p-tolylsulfonyloxy)ethoxy]ethoxy]piperidine-1-carboxylate (1.6 g, 95% yield).

### 1.5 Synthesis of Compound 6

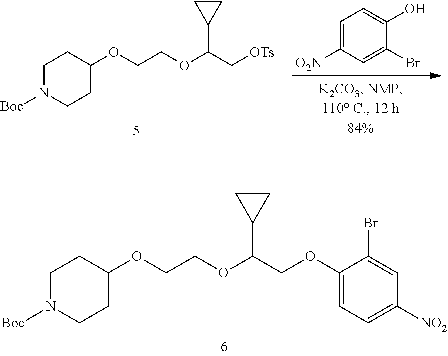 [SMILES: Cc1ccc(S(=O)(=O)OCC(OCCOC2CCN(C(=O)OC(C)(C)C)CC2)C2CC2)cc1 ; O=[N+]([O-])c1ccc(O)c(Br)c1 ; CC(C)(C)OC(=O)N1CCC(OCCOC(COc2ccc([N+](=O)[O-])cc2Br)C2CC2)CC1]

To a solution of tert-butyl 4-[2-[1-cyclopropyl-2-(p-tolylsulfonyloxy)ethoxy]ethoxy]piperidine-1-carboxylate (1.30 g, 2.69 μmol, 1.0 equiv) and 2-bromo-4-nitro-phenol (1.17 g, 5.4 μmol, 2.0 equiv) in NMP (10 mL) was added K₂CO₃ (743 mg, 5.4 μmol, 2.0 equiv). The mixture was stirred at 1100 C. for 12 h. The mixture was filtered and concentrated. The residue was purified by flash silica gel chromatography (ISCO®; 12 g SepaFlash® Silica Flash Column, Eluent of 0˜20% Ethyl acetate/Petroleum ethergradient at 60 mL/min) to give tert-butyl 4-[2-[2-(2-bromo-4-nitro-phenoxy)-1-cyclopropyl-ethoxy]ethoxy]piperidine-1-carboxylate (1.2 g, 84% yield). ¹H NMR (400 MHz, CDCl₃) δ 8.48 (d, J=2.8 Hz, 1H), 8.20 (s, 1H), 7.00 (d, J=8.8 Hz, 1H), 4.25-4.21 (m, 2H), 3.95-3.87 (m, 1H), 3.85-3.70 (m, 3H), 3.66-3.60 (m, 2H), 3.49 (s, 1H), 3.25 (s, 1H), 3.07 (s, 2H), 1.86-1.77 (m, 2H), 1.55-1.48 (m, 2H), 1.46 (s, 9H), 1.03-0.93 (m, 1H), 1.02-0.93 (m, 1H), 0.71-0.63 (m, 1H), 0.60-0.49 (m, 2H), 0.40-0.33 (m, 1H).

### 1.6 Synthesis of Compound 7

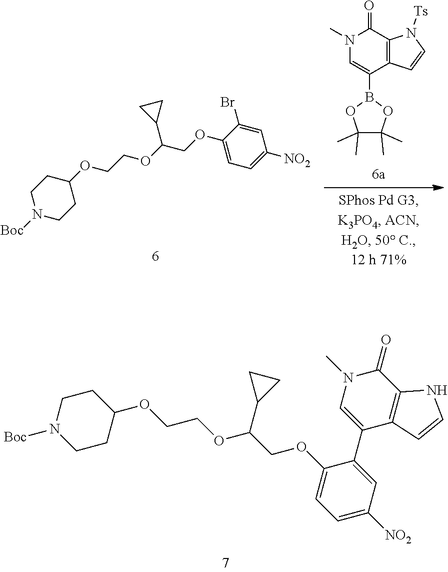 [SMILES: CC(C)(C)OC(=O)N1CCC(OCCOC(COc2ccc([N+](=O)[O-])cc2Br)C2CC2)CC1 ; Cc1ccc(S(=O)(=O)n2ccc3c(B4OC(C)(C)C(C)(C)O4)cn(C)c(=O)c32)cc1 ; Cn1cc(-c2cc([N+](=O)[O-])ccc2OCC(OCCOC2CCN(C(=O)OC(C)(C)C)CC2)C2CC2)c2cc[nH]c2c1=O]

To a solution of tert-butyl 4-[2-[2-(2-bromo-4-nitro-phenoxy)-1-cyclopropyl-ethoxy]ethoxy]piperidine-1-carboxylate (200 mg, 377 mol, 1.0 equiv) and 6-methyl-1-(p-tolylsulfonyl)-4-(4,4,5,5-tetramethyl-1,3,2-dioxaborolan-2-yl)pyrrolo[2,3-c]pyridin-7-one (161 mg, 377 μmol, 1.0 equiv) in ACN (2.5 mL) and H₂O (0.5 mL) was added SPhos Pd G3 (29 mg, 37 μmol, 0.1 equiv) and K₃PO₄ (240 mg, 1.13 μmol, 3.0 equiv). The mixture was stirred at 60° C. for 12 h. The mixture was filtered and concentrated. The residue was purified by prep-HPLC (column: Unisil 3-100 C18 Ultra 150 mm*50 mm*3 μm; mobile phase: [water(FA)-ACN]; B %: 55%-85%, 7 min) to give tert-butyl 4-[2-[1-cyclopropyl-2-[2-[6-methyl-7-oxo-1-(p-tolylsulfonyl)pyrrolo[2,3-c]pyridin-4-yl]-4-nitro-phenoxy]ethoxy]ethoxy]piperidine-1-carboxylate (200 mg, 71% yield).

### 1.7 Synthesis of Compound 8

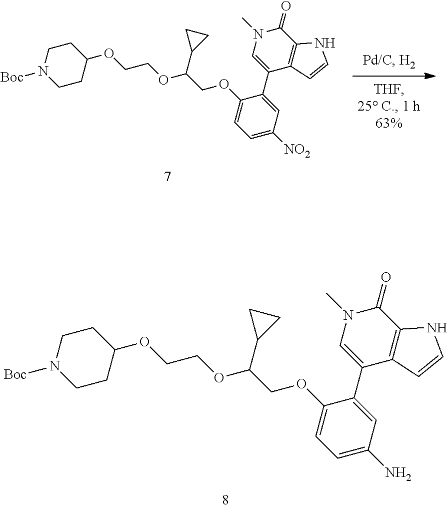 [SMILES: Cn1cc(-c2cc([N+](=O)[O-])ccc2OCC(OCCOC2CCN(C(=O)OC(C)(C)C)CC2)C2CC2)c2cc[nH]c2c1=O ; Cn1cc(-c2cc(N)ccc2OCC(OCCOC2CCN(C(=O)OC(C)(C)C)CC2)C2CC2)c2cc[nH]c2c1=O]

To a solution of tert-butyl 4-[2-[1-cyclopropyl-2-[2-[6-methyl-7-oxo-1-(p-tolylsulfonyl)pyrrolo[2,3-c]pyridin-4-yl]-4-nitro-phenoxy]ethoxy]ethoxy]piperidine-1-carboxylate (100 mg, 133 mol, 1.0 equiv) and Pd/C (10 mg, 410 μmol, 10% purity) in THF (2 mL). The mixture was stirred under H₂ (15 psi) at 20° C. for 1 h. The mixture was filtered and concentrated. The residue was purified by prep-TLC (SiO₂, DCM:MeOH=10:1). Tert-butyl 4-[2-[2-[4-amino-2-[6-methyl-7-oxo-1-(p-tolylsulfonyl)pyrrolo[2,3-c]pyridin-4-yl]phenoxy]-1-cyclopropyl-ethoxy]ethoxy]piperidine-1-carboxylate was obtained (80 mg, 83% yield).

### 1.8 Synthesis of 9

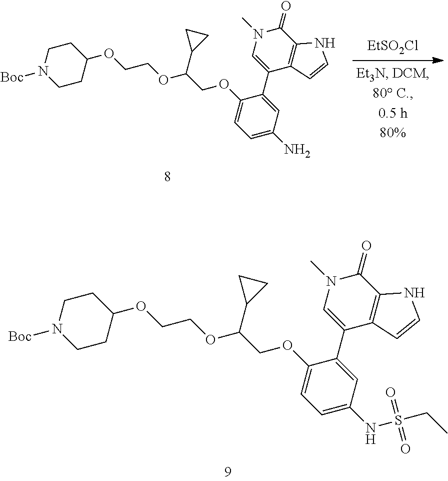 [SMILES: "EtSO2Cl" ; Cn1cc(-c2cc(N)ccc2OCC(OCCOC2CCN(C(=O)OC(C)(C)C)CC2)C2CC2)c2cc[nH]c2c1=O ; CCS(=O)(=O)Nc1ccc(OCC(OCCOC2CCN(C(=O)OC(C)(C)C)CC2)C2CC2)c(-c2cn(C)c(=O)c3[nH]ccc23)c1]

To a solution of tert-butyl 4-[2-[2-[4-amino-2-[6-methyl-7-oxo-1-(p-tolylsulfonyl) pyrrolo[2,3-c]pyridin-4-yl]phenoxy]-1-cyclopropyl-ethoxy]ethoxy]piperidine-1-carboxylate (80 mg, 110 mol, 1.0 equiv) in DCM (2 mL) was added TEA (34 mg, 332 μmol, 3.0 equiv) ethanesulfonyl chloride (28 mg, 221 μmol, 2.0 equiv). The mixture was stirred at 20° C. for 0.5 h and concentrated. The product was used directly for next step. Tert-butyl 4-[2-[2-[4-[bis(ethylsulfonyl)amino]-2-[6-methyl-7-oxo-1-(p-tolylsulfonyl)pyrrolo[2,3-c]pyridin-4-yl]phenoxy]-1-cyclopropyl-ethoxy]ethoxy]piperidine-1-carboxylate was obtained (80 mg, 80% yield).

### 1.9 Synthesis of Compound 10

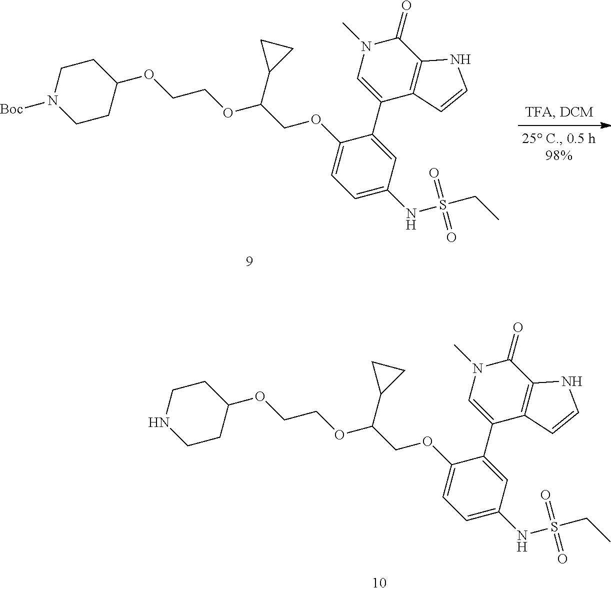 [SMILES: CCS(=O)(=O)Nc1ccc(OCC(OCCOC2CCN(C(=O)OC(C)(C)C)CC2)C2CC2)c(-c2cn(C)c(=O)c3[nH]ccc23)c1 ; CCS(=O)(=O)Nc1ccc(OCC(OCCOC2CCNCC2)C2CC2)c(-c2cn(C)c(=O)c3[nH]ccc23)c1]

To a solution of tert-butyl 4-[2-[1-cyclopropyl-2-[4-(ethylsulfonylamino)-2-(6-methyl-7-oxo-1H-pyrrolo[2,3-c]pyridin-4-yl)phenoxy]ethoxy]ethoxy]piperidine-1-carboxylate (20 mg, 30 μmol, 1.0 equiv) in DCM (1 mL) was added TFA (0.5 mL). The mixture was stirred at 25° C. for 0.5 h and concentrated. The product was used directly for next step.

### 2.0 Synthesis of I-68

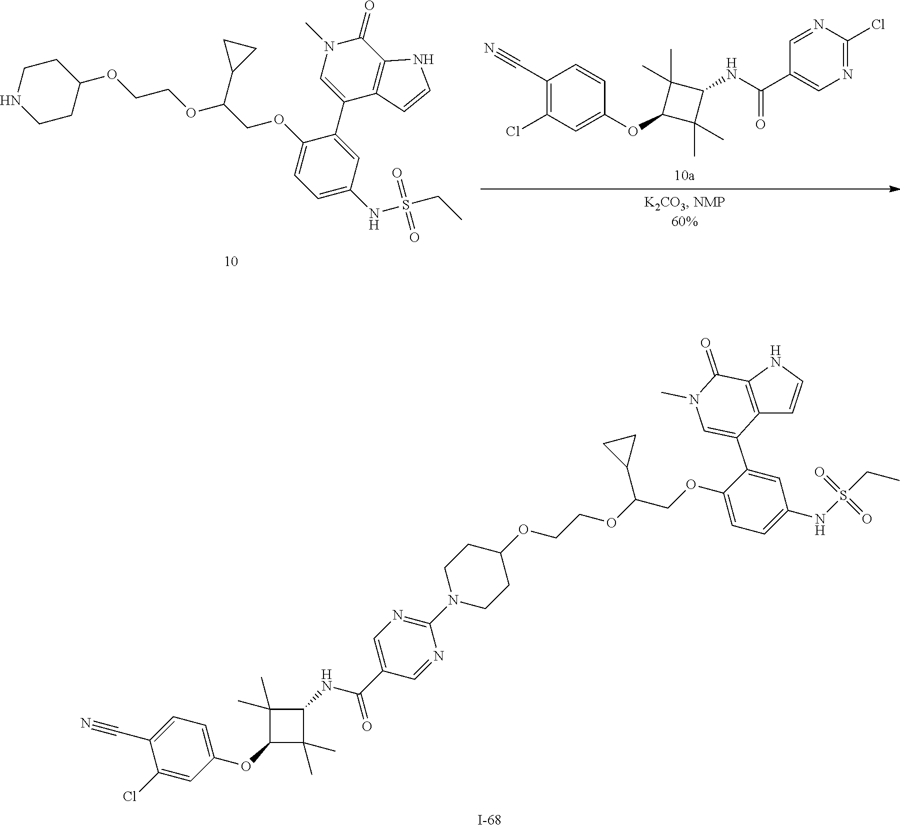

To a solution of N-[4-[2-cyclopropyl-2-[2-(4-piperidyloxy)ethoxy]ethoxy]-3-(6-methyl-7-oxo-1H-pyrrolo[2,3-c]pyridin-4-yl)phenyl]ethanesulfonamide (30 mg, 44 μmol, 1.0 equiv, TFA salt) in NMP (1 mL) was added K₂CO₃ (19 mg, 133 μmol, 3.0 equiv) and 2-chloro-N-1[3-(3-chloro-4-cyano-phenoxy)-2,2,4,4-tetramethyl-cyclobutyl]pyrimidine-5-carboxamide (18 mg, 44 mol, 1.0 equiv). The mixture was stirred at 50° C. for 1 h and concentrated. The residue was purified by prep-HPLC (column: Unisil 3-100 C18 Ultra 150 mm*50 mm*3 μm; mobile phase: [water(FA)-ACN]; B %: 52%-82%, 7 min) to give N-[3-(3-chloro-4-cyano-phenoxy)-2,2,4,4-tetramethyl-cyclobutyl]-2-[4-[2-[1-cyclopropyl-2-[4-(ethylsulfonylamino)-2-(6-methyl-7-oxo-1H-pyrrolo[2,3-c]pyridin-4-yl)phenoxy]ethoxy]ethoxy]-1-piperidyl]pyrimidine-5-carboxamide (28 mg, 60% yield). ¹H NMR (400 MHz, MeOD): δ 8.71 (s, 2H), 7.72 (d, J=8.8 Hz, 1H), 7.36 (s, 1H), 7.35 (d, J=2.8 Hz, 1H), 7.31 (d, J=2.8 Hz, 1H), 7.24 (s, 1H), 7.13 (d, J=2.4 Hz, 1H), 7.10 (d, J=8.9 Hz, 1H), 7.00-6.96 (m, 1H), 6.31 (d, J=2.8 Hz, 1H), 4.27 (s, 1H), 4.21-4.16 (m, 2H), 4.15-4.12 (m, 2H), 4.07-4.01 (m, 1H), 3.69 (s, 3H), 3.65-3.58 (m, 1H), 3.51-3.47 (m, 3H), 3.41 (s, 2H), 3.08 (s, 2H), 3.03-2.98 (m, 1H), 1.79 (s, 2H), 1.46-1.40 (m, 2H), 1.37-1.30 (m, 4H), 1.28 (s, 6H), 1.21 (s, 6H), 0.88-0.81 (m, 1H), 0.55-0.48 (m, 1H), 0.43-0.36 (m, 2H), 0.18-0.11 (m, 1H).

### Example 18—Synthesis of N-((1r, 3r)-3-(3-chloro-4-cyanophenoxy)-2,2,4,4-tetramethylcyclobutyl)-4-(4-(2-(3-(4-(ethylsulfonamido)-2-(6-methyl-7-oxo-6,7-dihydro-1H-pyrrolo[2,3-c]pyridin-4-yl)phenoxy)phenoxy)ethoxy)piperidin-1-yl)benzamide (I-69)

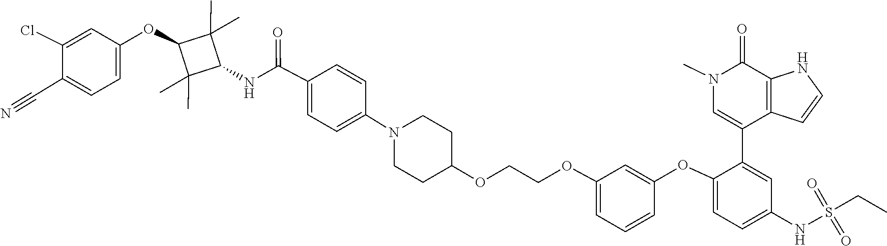 [SMILES: CCS(=O)(=O)Nc1ccc(Oc2cccc(OCCOC3CCN(c4ccc(C(=O)N[C@H]5C(C)(C)[C@H](Oc6ccc(C#N)c(Cl)c6)C5(C)C)cc4)CC3)c2)c(-c2cn(C)c(=O)c3[nH]ccc23)c1]

### 1.1 Synthesis of Compound 2

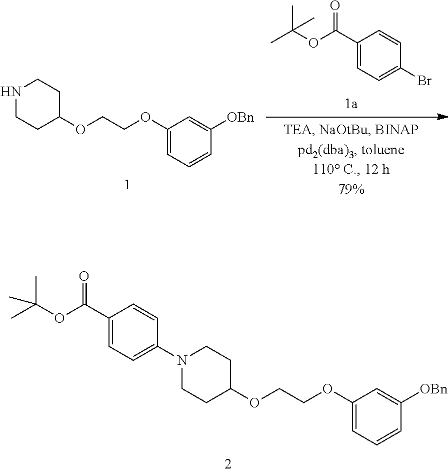 [SMILES: c1ccc(COc2cccc(OCCOC3CCNCC3)c2)cc1 ; CC(C)(C)OC(=O)c1ccc(Br)cc1 ; CC(C)(C)OC(=O)c1ccc(N2CCC(OCCOc3cccc(OCc4ccccc4)c3)CC2)cc1]

A mixture of 4-[2-(3-benzyloxyphenoxy)ethoxy]piperidine (1 g, 3 μmol, 1.1 equiv, HCl salt), tert-butyl 4-bromobenzoate (642 mg, 2.5 mol, 1.0 equiv), TEA (278 mg, 3 mol, 1.1 equiv), NaOtBu (792 mg, 8 mol, 3.3 equiv), BINAP (156 mg, 250 μmol, 0.1 equiv) and Pd₂(dba)₃ (114 mg, 125 mol, 0.05 equiv) in toluene (10 mL) was degassed and purged with N₂ for three times. The mixture was stirred at 1100 C. for 12 h under N₂ atmosphere and concentrated. The residue was purified by column chromatography (SiO₂, Petroleum ether/Ethyl acetate=20/1 to 3/1) to give tert-butyl 4-[4-[2-(3-benzyloxyphenoxy)ethoxy]-1-piperidyl]benzoate (1 g, 79% yield) as a yellow oil. ¹H NMR (400 MHz, DMSO-d₆): δ 7.70 (d, J=8.6 Hz, 2H), 7.36-7.45 (m, 4H), 7.30-7.35 (m, 1H), 7.13-7.20 (m, 1H), 6.95 (d, J=8.8 Hz, 2H), 6.56-6.60 (m, 2H), 6.53 (d, J=8.2 Hz, 1H), 5.07 (s, 2H), 4.05-4.09 (m, 2H), 3.73-3.80 (m, 2H), 3.59-3.66 (m, 2H), 3.27-3.34 (m, 2H), 3.02-3.10 (m, 2H), 1.88-1.94 (m, 2H), 1.51 (s, 9H), 1.45-1.49 (m, 1H).

### 1.2 Synthesis of Compound 3

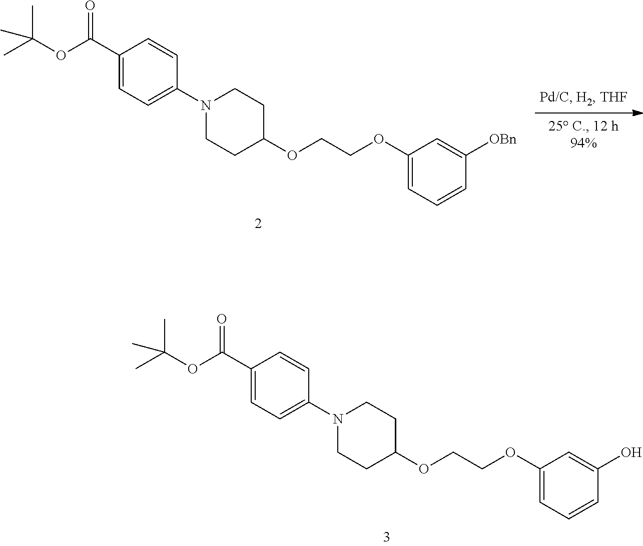 [SMILES: CC(C)(C)OC(=O)c1ccc(N2CCC(OCCOc3cccc(OCc4ccccc4)c3)CC2)cc1 ; CC(C)(C)OC(=O)c1ccc(N2CCC(OCCOc3cccc(O)c3)CC2)cc1]

To a solution of tert-butyl 4-[4-[2-(3-benzyloxyphenoxy)ethoxy]-1-piperidyl]benzoate (1 g, 2 gmol, 1.0 equiv) in THF (10 mL) was added Pd/C (100 mg, 10% purity) under N₂ atmosphere. The mixture was stirred under H₂ (15 Psi) at 25° C. for 12 h. The mixture was filtered and concentrated. The residue was purified by column chromatography (SiO₂, Petroleum ether/Ethyl acetate=10/1 to 3/1) to give tert-butyl 4-[4-[2-(3-hydroxy-phenoxy)ethoxy]-1-piperidyl]benzoate (770 mg, 94% yield) as a yellow oil. ¹H NMR (400 MHz, CDCl₃): δ 7.86 (d, J=8.8 Hz, 2H), 7.09-7.15 (m, 1H), 6.86 (d, J=8.8 Hz, 2H), 6.48-6.53 (m, 1H), 6.41-6.46 (m, 2H), 4.98-5.06 (m, 1H), 4.12-4.17 (m, 1H), 3.83-3.87 (m, 2H), 3.64-3.70 (m, 2H), 3.07-3.14 (m, 2H), 1.98-2.03 (m, 2H), 1.71-1.78 (m, 2H), 1.59 (s, 2H), 1.58 (s, 9H).

### 1.3 Synthesis of Compound 4

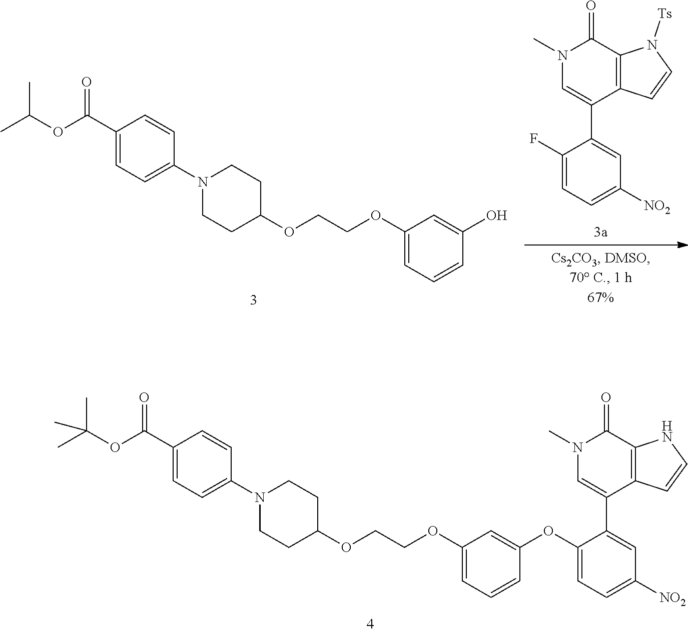 [SMILES: CC(C)OC(=O)c1ccc(N2CCC(OCCOc3cccc(O)c3)CC2)cc1 ; Cc1ccc(S(=O)(=O)n2ccc3c(-c4cc([N+](=O)[O-])ccc4F)cn(C)c(=O)c32)cc1 ; Cn1cc(-c2cc([N+](=O)[O-])ccc2Oc2cccc(OCCOC3CCN(c4ccc(C(=O)OC(C)(C)C)cc4)CC3)c2)c2cc[nH]c2c1=O]

To a solution of tert-butyl 4-[4-[2-(3-hydroxyphenoxy)ethoxy]-1-piperidyl]benzoate (258 mg, 623 μmol, 1.1 equiv) in NMP (3 mL) was added Cs₂CO₃ (369 mg, 1 mol, 2.0 equiv) and 4-(2-fluoro-5-nitro-phenyl)-6-methyl-1-(p-tolylsulfonyl)pyrrolo [2,3-c]pyridin-7-one (250 mg, 566 mol, 1.0 equiv). The mixture was stirred at 70° C. for 1 h and concentrated. The residue was purified by prep-TLC (SiO₂, PE: EA=1/2) to give tert-butyl 4-[4-[2-[3-[2-(6-methyl-7-oxo-1H-pyrrolo[2,3-c]pyridin-4-yl)-4-nitro-phenoxy]phenoxy]ethoxy]-1-piperidyl]benzoate (260 mg, 67% yield). ¹H NMR (400 MHz, DMSO-d₆): δ 12.04-12.16 (m, 1H), 8.31 (d,. J=2.4 Hz, 1H), 8.20-8.23 (m, 1H), 7.69 (d, J=8.4 Hz, 2H), 7.47 (s, 1H), 7.26-7.36 (m, 2H), 6.99-7.05 (m, 1H), 6.90-6.97 (m, 2H), 6.83 (d, J=8.4 Hz, 1H), 6.69-6.79 (m, 2H), 6.32 (s, 1H), 4.06-4.12 (m, 4H), 3.75 (s, 2H), 3.61-3.65 (m, 2H), 3.57 (s, 3H), 3.01-3.09 (m, 2H), 1.85-1.94 (m, 2H), 1.50 (s, 9H), 1.42-1.47 (m, 1H).

### 1.4 Synthesis of Compound 5

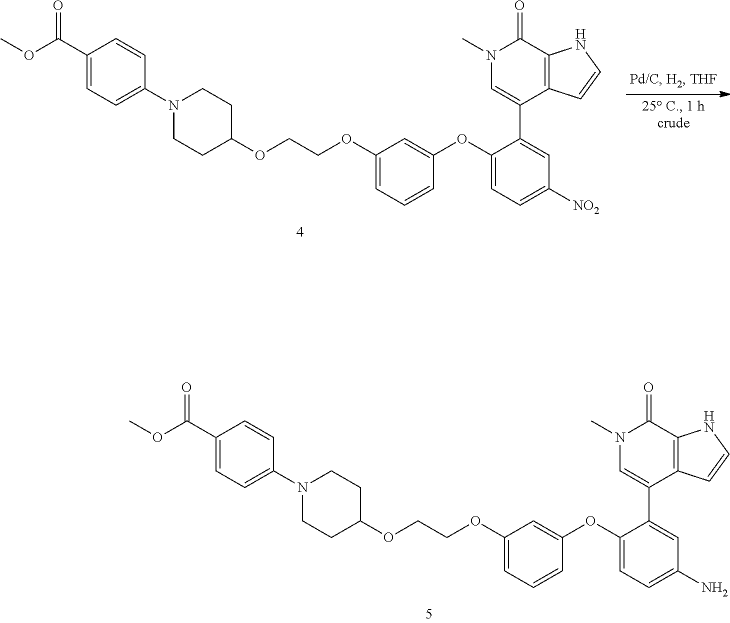 [SMILES: COC(=O)c1ccc(N2CCC(OCCOc3cccc(Oc4ccc([N+](=O)[O-])cc4-c4cn(C)c(=O)c5[nH]ccc45)c3)CC2)cc1 ; COC(=O)c1ccc(N2CCC(OCCOc3cccc(Oc4ccc(N)cc4-c4cn(C)c(=O)c5[nH]ccc45)c3)CC2)cc1]

To a solution of tert-butyl 4-[4-[2-[3-[2-(6-methyl-7-oxo-1H-pyrrolo[2,3-c]pyridin-4-yl)-4-nitro-phenoxy]phenoxy]ethoxy]-1-piperidyl]benzoate (48 mg, 71 μmol, 1.0 equiv) in THF (3 mL) was added Pd/C (10 mg, 10% purity) under N₂ atmosphere. The mixture was stirred at 25° C. for 1 h. The mixture was filtered and concentrated to give tert-butyl4-[4-[2-[3-[4-amino-2-(6-methyl-7-oxo-1H-pyrrolo[2,3-c]pyridin-4-yl)phenoxy]phenoxy]ethoxy]-1-piperidyl]benzoate (45 mg, crude).

### 1.5 Synthesis of Compound 6

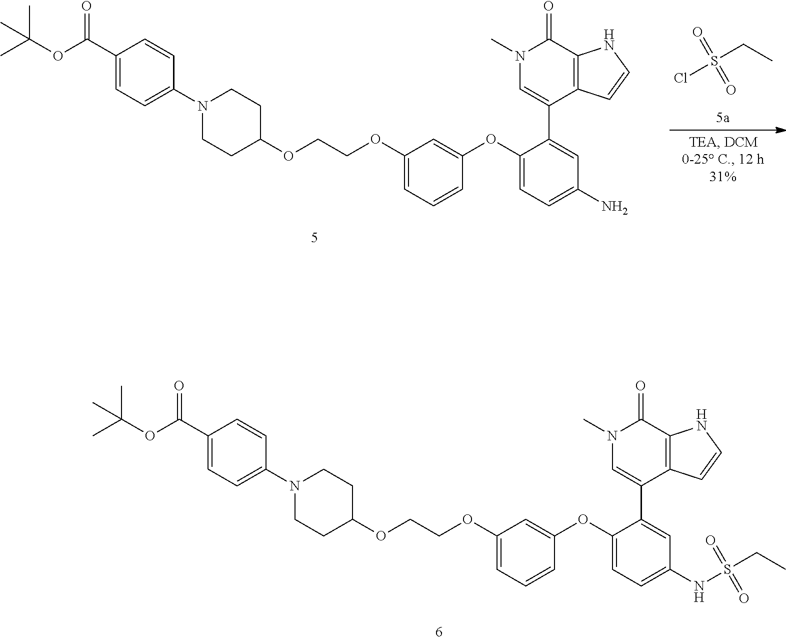

To a solution of tert-butyl 4-[4-[2-[3-[4-amino-2-(6-methyl-7-oxo-1H-pyrrolo[2,3-c]pyridin-4-yl)phenoxy]phenoxy]ethoxy]-1-piperidyl]benzoate (45 mg, 69 mol, 1.0 equiv) in DCM (1 mL) was added TEA (84 mg, 830 mol, 12.0 equiv) and ethanesulfonyl chloride (27 mg, 207 mol, 3.0 equiv) at 0° C. The mixture was stirred at 25° C. for 12 h and concentrated. The residue was purified by prep-TLC (SiO₂, DCM: MeOH=20/1) to give tert-butyl 4-[4-[2-[3-[4-(ethylsulfonylamino)-2-(6-methyl-7-oxo-1H-pyrrolo [2,3-c]pyridin-4-yl)phenoxy]phenoxy]ethoxy]-1-piperidyl]benzoate (16 mg, 31% yield).

### 1.6 Synthesis of Compound 7

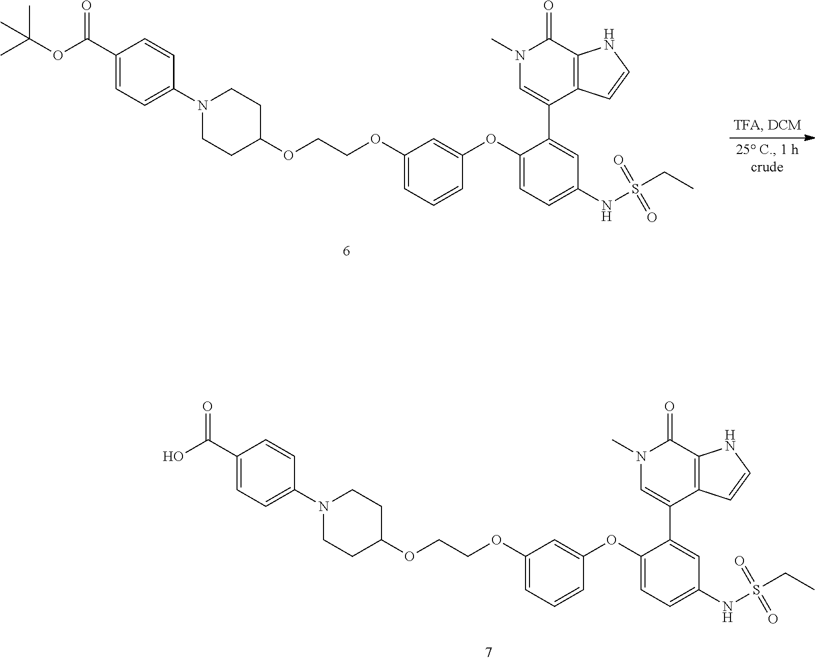 [SMILES: CCS(=O)(=O)Nc1ccc(Oc2cccc(OCCOC3CCN(c4ccc(C(=O)OC(C)(C)C)cc4)CC3)c2)c(-c2cn(C)c(=O)c3[nH]ccc23)c1 ; CCS(=O)(=O)Nc1ccc(Oc2cccc(OCCOC3CCN(c4ccc(C(=O)O)cc4)CC3)c2)c(-c2cn(C)c(=O)c3[nH]ccc23)c1]

To a solution of tert-butyl 4-[4-[2-[3-[4-(ethylsulfonylamino)-2-(6-methyl-7-oxo-1H-pyrrolo[2,3-c]pyridin-4-yl)phenoxy]phenoxy]ethoxy]-1-piperidyl]benzoate (16 mg, 22 μmol, 1.0 equiv) in DCM (0.5 mL) was added TFA (0.1 mL). The mixture was stirred at 25° C. for 1 h and concentrated to give a crude product.

### 1.7 Synthesis of I-69

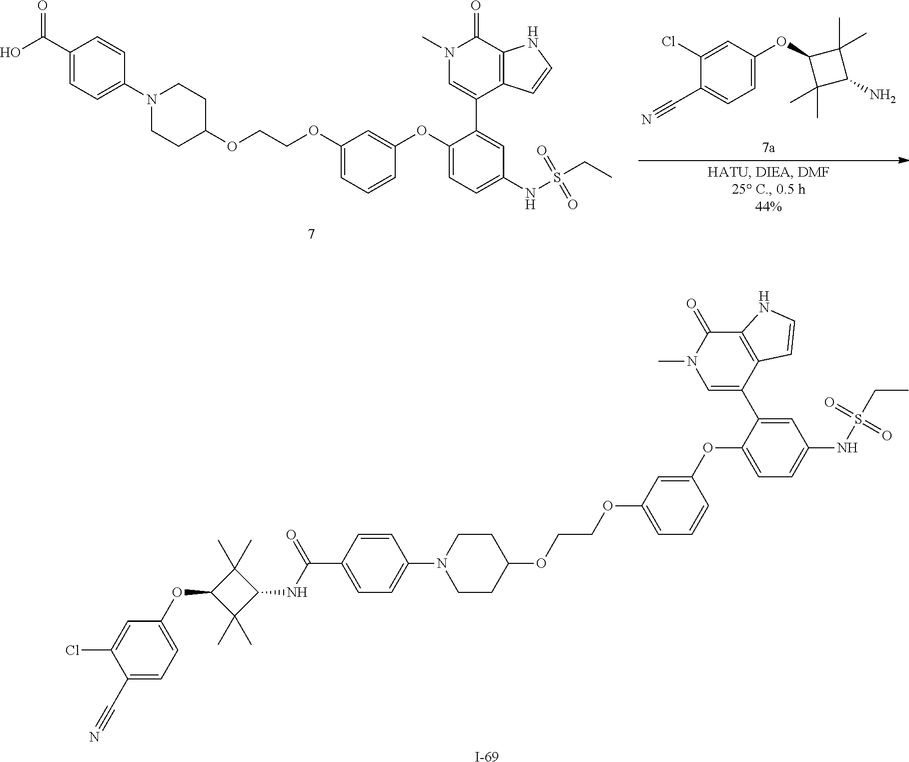

To a solution of 4-[4-[2-[3-[4-(ethylsulfonylamino)-2-(6-methyl-7-oxo-1H-pyrrolo[2,3-c]pyridin-4-yl)phenoxy]phenoxy]ethoxy]-1-piperidyl]benzoic acid (17 mg, 21 μmol, 1.0 equiv) in DMF (1 mL) was added 4-(3-amino-2,2,4,4-tetramethyl-cyclobutoxy)-2-chloro-benzonitrile (8 mg, 21 mol, 1.0 equiv, TFA salt), DIEA (14 mg, 106 mol, 5.0 equiv) and HATU (11 mg, 28 μmol, 1.3 equiv). The mixture was stirred at 25° C. for 0.5 h and concentrated. The residue was purified by prep-HPLC (column: Waters Xbridge 150 mm*25 mm*5 μm; mobile phase: [water(NH₄HCO₃)-ACN]; B %: 50%-80%, 9 min) to give N-[3-(3-chloro-4-cyano-phenoxy)-2,2,4,4-tetramethyl-cyclobutyl]-4-[4-[2-[3-[4-(ethylsulfonylamino)-2-(6-methyl-7-oxo-1H-pyrrolo[2,3-c]pyridin-4-yl)phenoxy]phenoxy]ethoxy]-1-piperidyl]benzamide (9 mg, 44% yield) as a white solid. ¹H NMR (400 MHz, CDCl₃): δ 9.95 (s, 1H), 7.68 (d, J=8.8 Hz, 2H), 7.57 (d, J=8.8 Hz, 1H), 7.47 (d, J=2.6 Hz, 1H), 7.39 (s, 1H), 7.21-7.24 (m, 2H), 7.13 (t, J=8.2 Hz, 1H), 7.02-7.07 (m, 2H), 6.97 (d, J=2.4 Hz, 1H), 6.89 (d, J=8.8 Hz, 2H), 6.80-6.83 (m, 1H), 6.56-6.59 (m, 1H), 6.45-6.48 (m, 1H), 6.41-6.43 (m, 2H), 6.17 (d, J=8.0 Hz, 1H), 4.15-4.17 (m, 1H), 4.04-4.06 (m, 1H), 4.03-4.05 (m, 1H), 3.77-3.83 (m, 2H), 3.60-3.64 (m, 2H), 3.58 (s, 3H), 3.17-3.22 (m, 2H), 3.03-3.13 (m, 2H), 1.93-2.01 (m, 2H), 1.69-1.77 (m, 2H), 1.64-1.68 (m, 2H), 1.43 (t, J=7.4 Hz, 3H), 1.27 (s, 6H) 1.22 (s, 6H).

### Example 19—Synthesis of N-((1r, 3r)-3-(3-chloro-4-cyanophenoxy)-2,2,4,4-tetramethylcyclobutyl)-6-(4-(2-(3-(4-(ethylsulfonamido)-2-(6-methyl-7-oxo-6,7-dihydro-1H-pyrrolo[2,3-c]pyridin-4-yl)phenoxy)phenoxy)ethoxy)piperidin-1-yl)nicotinamide (I-70)

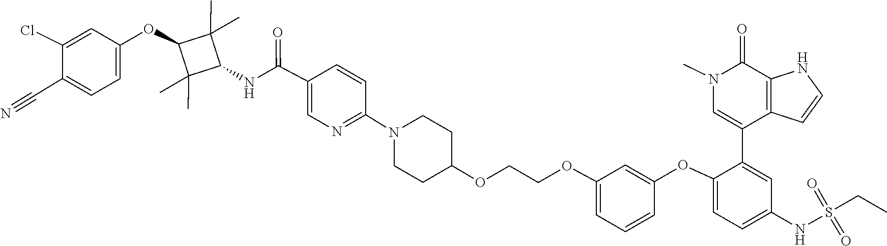 [SMILES: CCS(=O)(=O)Nc1ccc(Oc2cccc(OCCOC3CCN(c4ccc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1]

### 1.1 Synthesis of Compound 2

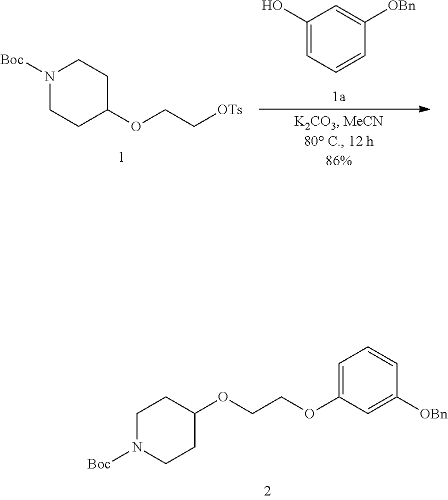 [SMILES: Cc1ccc(S(=O)(=O)OCCOC2CCN(C(=O)OC(C)(C)C)CC2)cc1 ; Oc1cccc(OCc2ccccc2)c1 ; CC(C)(C)OC(=O)N1CCC(OCCOc2cccc(OCc3ccccc3)c2)CC1]

To a solution of 3-benzyloxyphenol (7.17 g, 35.79 mol, 1.1 equiv) in ACN (150 mL) was added K₂CO₃ (13.5 g, 98 μmol, 3.0 equiv) and tert-butyl 4-[2-(p-tolylsulfonyloxy) ethoxy]piperidine-1-carboxylate (13 g, 32.5 μmol, 1.0 equiv). The mixture was stirred at 80° C. for 12 h and concentrated. The residue was purified by prep-HPLC (column: Welch Ultimate XB—CN 250 mm*50 mm*10 μm; mobile phase: [Hexane-EtOH]; B %: 1%-25%, 15 min) to give tert-butyl 4-[2-(3-benzyloxyphenoxy) ethoxy]piperidine-1-carboxylate (12 g, 86% yield). ¹H NMR (400 MHz, CDCl₃) δ 7.38-7.18 (m, 5H), 7.10 (d, J=8.4 Hz, 1H), 6.56-6.41 (m, 3H), 4.97 (s, 2H), 4.02 (m, 2H), 3.73 (m, 2H), 3.48 (m, 1H), 3.02 (m, 2H), 1.77 (m, 2H), 1.53-1.45 (m, 2H), 1.39 (s, 9H), 1.22-1.15 (m, 1H), 0.81 (m, 1H).

### 1.2 Synthesis of Compound 3

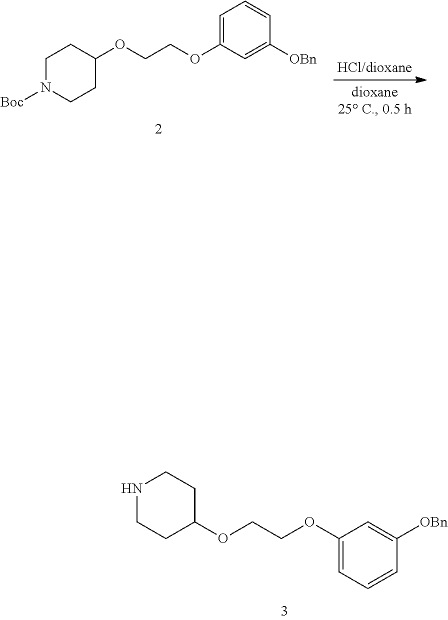 [SMILES: CC(C)(C)OC(=O)N1CCC(OCCOc2cccc(OCc3ccccc3)c2)CC1 ; c1ccc(COc2cccc(OCCOC3CCNCC3)c2)cc1]

To a solution of tert-butyl 4-[2-(3-benzyloxyphenoxy)ethoxy]piperidine-1-carboxylate (1 g, 2.34 mol, 1.0 equiv) in dioxane (2 mL) was added HCl/dioxane (4 M, 10 mL). The mixture was stirred at 25° C. for 0.5 h. The reaction mixture was filtered and concentrated to give 4-[2-(3-benzyloxyphenoxy)ethoxy]piperidine (700 mg, crude, HCl salt).

### 1.3 Synthesis of Compound 4

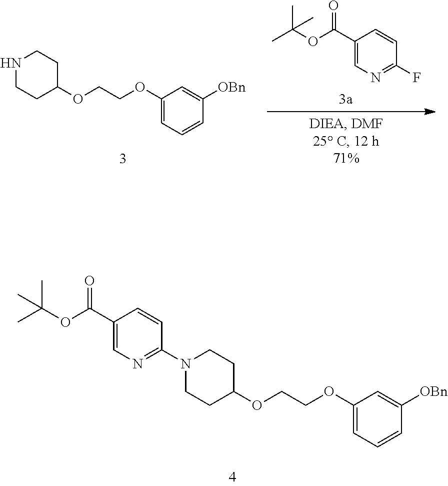 [SMILES: c1ccc(COc2cccc(OCCOC3CCNCC3)c2)cc1 ; CC(C)(C)OC(=O)c1ccc(F)nc1 ; CC(C)(C)OC(=O)c1ccc(N2CCC(OCCOc3cccc(OCc4ccccc4)c3)CC2)nc1]

To a solution of 4-[2-(3-benzyloxyphenoxy)ethoxy]piperidine (100 mg, 305 μmol, 1.0 equiv) in DMF (3 mL) was added DIEA (118 mg, 916 μmol, 3.0 equiv) and tert-butyl 6-fluoropyridine-3-carboxylate (60 mg, 305 μmol, 1.0 equiv). The mixture was stirred at 25° C. for 12 h and concentrated. The residue was purified by prep-TLC (SiO₂, PE: EA=10:1) to give tert-butyl 6-[4-[2-(3-benzyloxyphenoxy) ethoxy]-1-piperidyl]pyridine-3-carboxylate (110 mg, 71% yield).

### 1.4 Synthesis of Compound 5

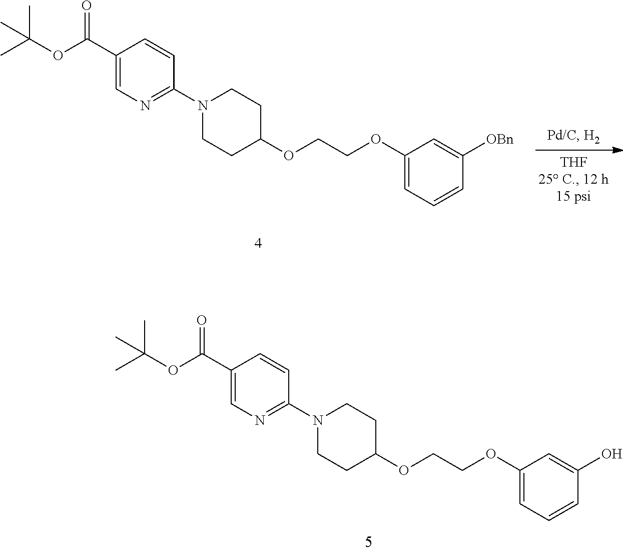 [SMILES: CC(C)(C)OC(=O)c1ccc(N2CCC(OCCOc3cccc(OCc4ccccc4)c3)CC2)nc1 ; CC(C)(C)OC(=O)c1ccc(N2CCC(OCCOc3cccc(O)c3)CC2)nc1]

To a solution tert-butyl 6-[4-[2-(3-benzyloxyphenoxy)ethoxy]-1-piperidyl]pyridine-3-carboxylate (110 mg, 218 mol, 1.0 equiv) in THF (1 mL) was added Pd/C (0.01 g, 10% purity). The mixture was stirred under H₂ (15 psi) at 20° C. for 12 h. The mixture was filtered and concentrated to give tert-butyl 6-[4-[2-(3-hydroxyphenoxy) ethoxy]-1-piperidyl]pyridine-3-carboxylate (900 mg, crude).

### 1.5 Synthesis of Compound 6

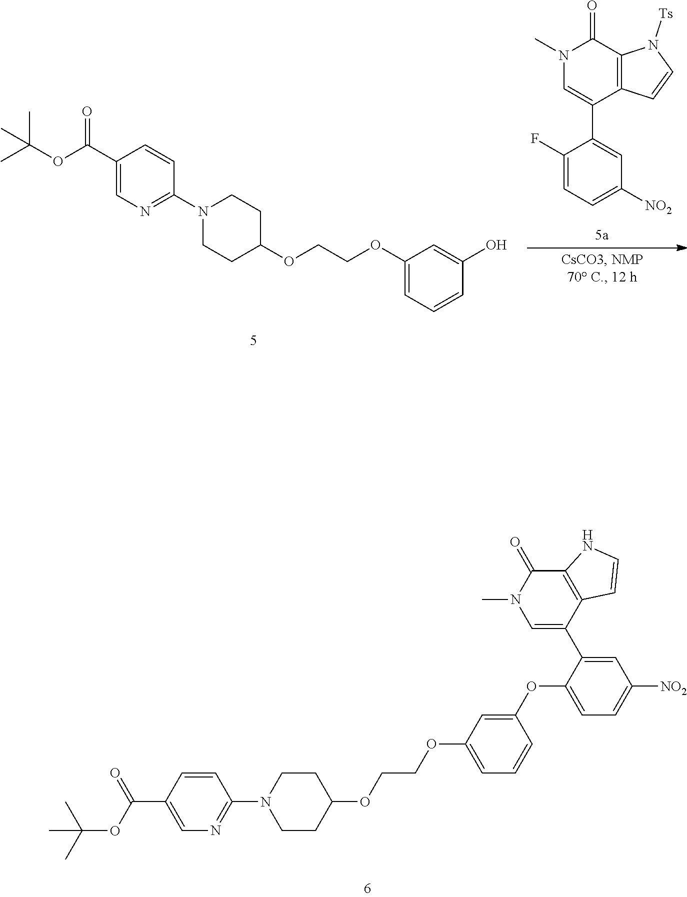 [SMILES: CC(C)(C)OC(=O)c1ccc(N2CCC(OCCOc3cccc(O)c3)CC2)nc1 ; Cc1ccc(S(=O)(=O)n2ccc3c(-c4cc([N+](=O)[O-])ccc4F)cn(C)c(=O)c32)cc1 ; Cn1cc(-c2cc([N+](=O)[O-])ccc2Oc2cccc(OCCOC3CCN(c4ccc(C(=O)OC(C)(C)C)cn4)CC3)c2)c2cc[nH]c2c1=O]

To a solution of tert-butyl 6-[4-[2-(3-hydroxyphenoxy)ethoxy]-1-piperidyl]pyridine-3-carboxylate (100 mg, 241 mol, 1.0 equiv) in NMP (1 mL) was added Cs₂CO₃ (196 mg, 603 μmol, 2.5 equiv) and 4-(2-fluoro-5-nitro-phenyl)-6-methyl-1-(p-tolylsulfonyl) pyrrolo[2,3-c]pyridin-7-one (106 mg, 241 mol, 1.0 equiv). The mixture was stirred at 70° C. for 12 h. The reaction mixture was filtered and concentrated. To give tert-butyl 6-[4-[2-[3-[2-(6-methyl-7-oxo-1H-pyrrolo [2, 3-c]pyridin-4-yl)-4-nitro-phenoxy]phenoxy]ethoxy]-1-piperidyl]pyridine-3-carboxylate (90 mg, crude).

### 1.6 Synthesis of Compound 7

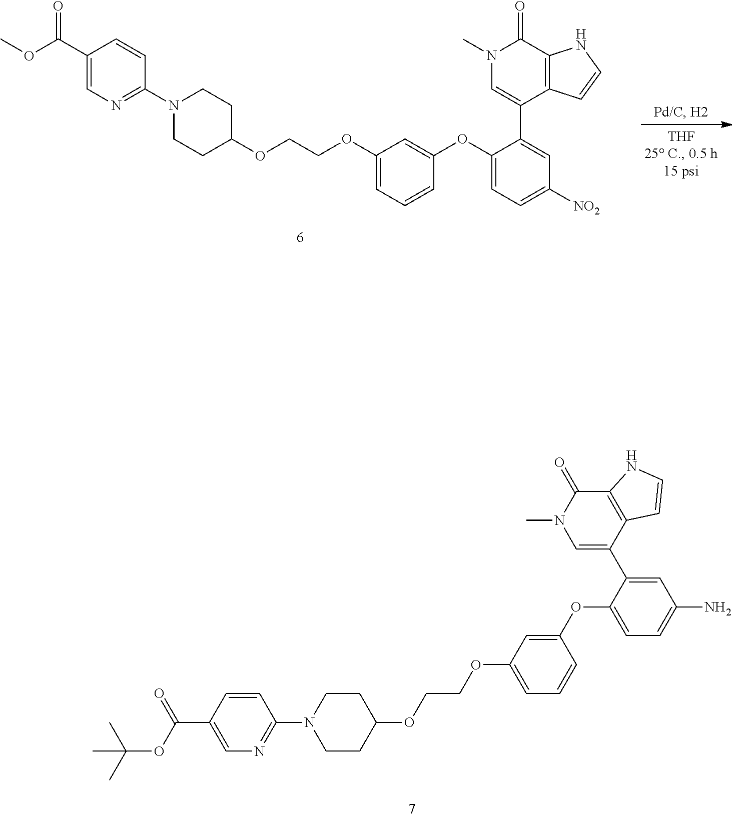 [SMILES: COC(=O)c1ccc(N2CCC(OCCOc3cccc(Oc4ccc([N+](=O)[O-])cc4-c4cn(C)c(=O)c5[nH]ccc45)c3)CC2)nc1 ; Cn1cc(-c2cc(N)ccc2Oc2cccc(OCCOC3CCN(c4ccc(C(=O)OC(C)(C)C)cn4)CC3)c2)c2cc[nH]c2c1=O]

To a solution of tert-butyl 6-[4-[2-[3-[2-(6-methyl-7-oxo-1H-pyrrolo[2,3-c]pyridin-4-yl)-4-nitro-phenoxy]phenoxy]ethoxy]-1-piperidyl]pyridine-3-carboxylate (90 mg, 132 μmol, 1.0 equiv) in THF (2 mL) was added Pd/C (0.01 g, 10% purity). The mixture was stirred under H₂ (15 psi) at 25° C. for 0.5 h. The mixture was filtered and concentrated to give tert-butyl 6-[4-[2-[3-[4-amino-2-(6-methyl-7-oxo-1H-pyrrolo[2,3-c]pyridin-4-yl)phenoxy]phenoxy]ethoxy]-1-piperidyl]pyridine-3-carboxylate (44 mg, crude).

### 1.7 Synthesis of Compound 8

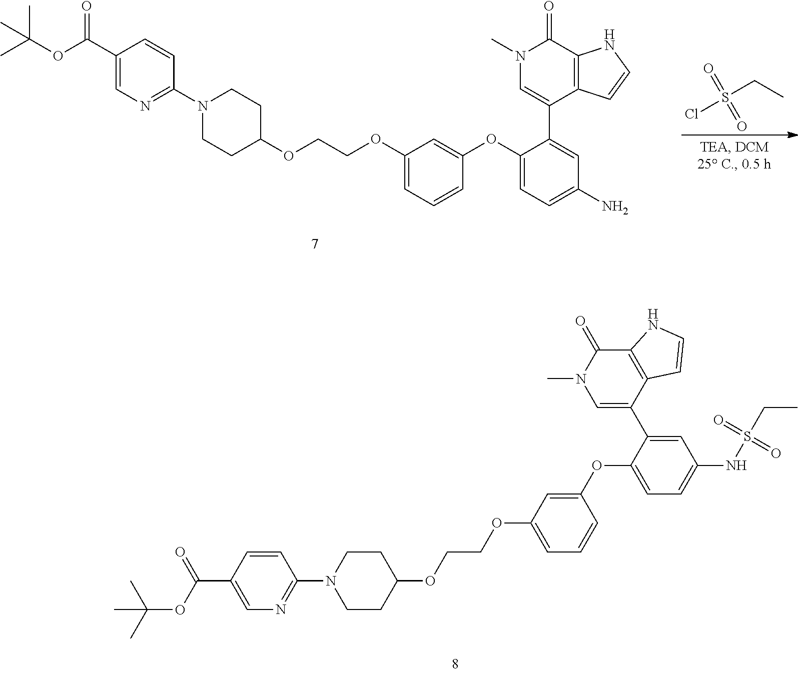

To a solution of tert-butyl 6-[4-[2-[3-[4-amino-2-(6-methyl-7-oxo-1H-pyrrolo[2,3-c]pyridin-4-yl)phenoxy]phenoxy]ethoxy]-1-piperidyl]pyridine-3-carboxylate (36 mg, 55 μmol, 1.0 equiv) in DCM (3 mL) was added TEA (28 mg, 273 gmol, 5.0 equiv) and ethanesulfonyl chloride (21 mg, 164 mol, 3.0 equiv). The mixture was stirred at 25° C. for 0.5 h and concentrated. The residue was purified by prep-HPLC (column: Waters Xbridge 150 mm*25 mm*5 μm; mobile phase: [water(NH₄HCO₃)-ACN]; B %: 49%-79%, 9 min) to give tert-butyl 6-[4-[2-[3-[4-(ethylsulfonylamino)-2-(6-methyl-7-oxo-1H-pyrrolo[2,3-c]pyridin-4-yl)phenoxy]phenoxy]ethoxy]-1-piperidyl]pyridine-3-carboxylate (20 mg, crude).

### 1.8 Synthesis of Compound 9

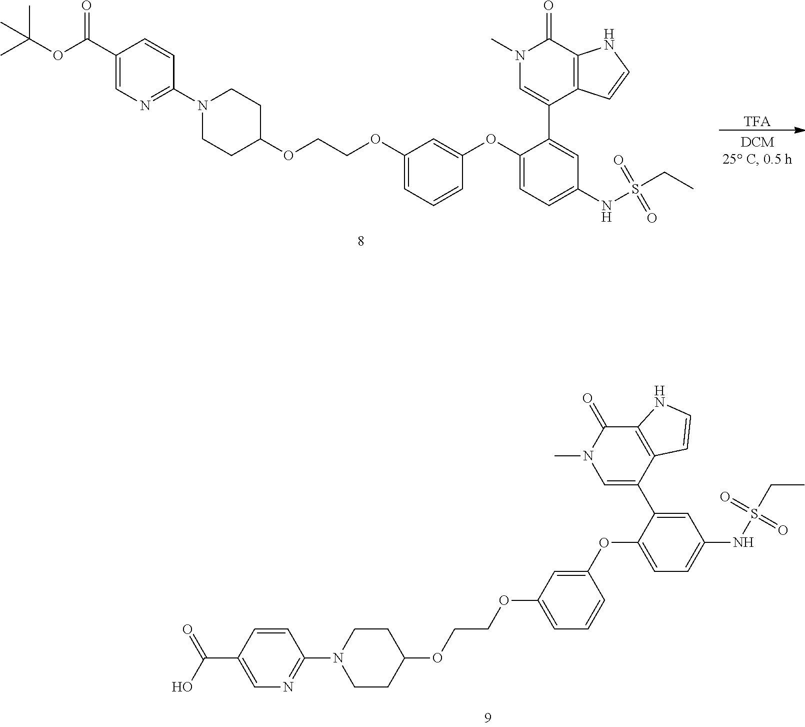 [SMILES: CCS(=O)(=O)Nc1ccc(Oc2cccc(OCCOC3CCN(c4ccc(C(=O)OC(C)(C)C)cn4)CC3)c2)c(-c2cn(C)c(=O)c3[nH]ccc23)c1 ; CCS(=O)(=O)Nc1ccc(Oc2cccc(OCCOC3CCN(c4ccc(C(=O)O)cn4)CC3)c2)c(-c2cn(C)c(=O)c3[nH]ccc23)c1 ; "TFA"]

To a solution of tert-butyl 6-[4-[2-[3-[4-(ethylsulfonylamino)-2-(6-methyl-7-oxo-1H-pyrrolo[2,3-c]pyridin-4-yl)phenoxy]phenoxy]ethoxy]-1-piperidyl]pyridine-3-carboxylate (22 mg, 30 μmol, 1.0 equiv) in DCM (1 mL) was added TFA (0.5 mL). The mixture was stirred at 25° C. for 0.5 h and concentrated. The product was used directly for next step.

### 1.9 Synthesis of I-70

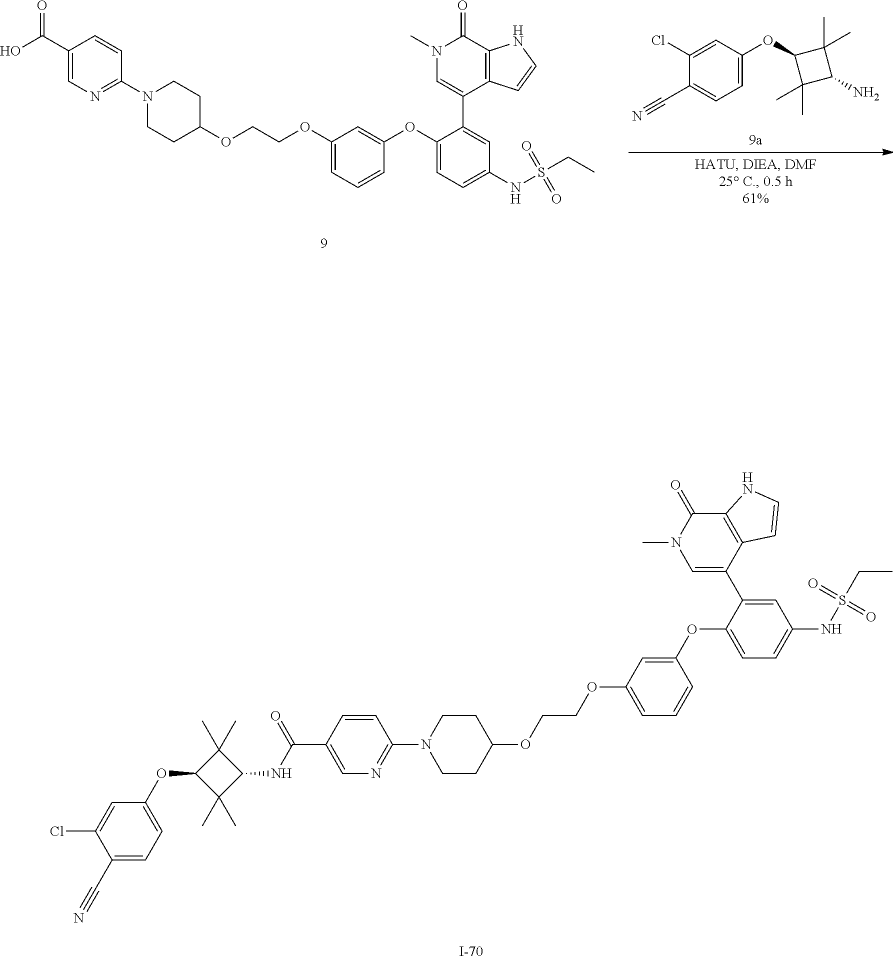

To a solution of 6-[4-[2-[3-[4-(ethylsulfonylamino)-2-(6-methyl-7-oxo-1H-pyrrolo[2,3-c]pyridin-4-yl)phenoxy]phenoxy]ethoxy]-1-piperidyl]pyridine-3-carboxylic acid (23 mg, 33 mol, 1.0 equiv) in DMF (1 mL) was added HATU (17 mg, 43 mol, 1.3 equiv) and DIEA (22 mg, 167 μmol, 5.0 equiv) 4-(3-amino-2,2,4,4-tetramethyl-cyclobutoxy)-2-chloro-benzonitrile (9.3 mg, 33 mol, 1.0 equiv). The mixture was stirred at 25° C. for 0.5 h and concentrated. The residue was purified by prep-HPLC (column: Phenomenex C18 75 mm*30 mm*3 μm; mobile phase: [water(FA)-ACN]; B %: 28%-58%, 7 min) (column: Phenomenex C18 75 mm*30 mm*3 μm; mobile phase: [water(FA)-ACN]; B %: 50%-80%, 7 min) to give N-[3-(3-chloro-4-cyano-phenoxy)-2,2,4,4-tetramethyl-cyclobutyl]-6-[4-[2-[3-[4-(ethylsulfonylamino)-2-(6-methyl-7-oxo-1H-pyrrolo[2,3-c]pyridin-4-yl)phenoxy]phenoxy]ethoxy]-1-piperidyl]pyridine-3-carboxamide (20 mg, 61% yield). ¹H NMR (400 MHz, DMSO-d₆) δ 12.05 (s, 1H), 10.03-9.55 (m, 1H), 8.62 (d, J=2.1 Hz, 1H), 8.15 (s, 1H), 8.00-7.80 (m, 2H), 7.60 (d, J=9.2 Hz, 1H), 7.41 (d, J=2.8 Hz, 1H), 7.34-7.25 (m, 2H), 7.25-7.18 (m, 2H), 7.14 (m, 1H), 7.06 (d, J=8.8 Hz, 1H), 7.01 (m, 1H), 6.92-6.79 (m, 1H), 6.62-6.52 (m, 1H), 6.44-6.37 (m, 2H), 6.27 (m, 1H), 4.31 (s, 1H), 4.20-3.84 (m, 5H), 3.77-3.67 (m, 2H), 3.65-3.59 (m, 1H), 3.49 (s, 3H), 3.27-3.21 (m, 2H), 3.13 (m, 2H), 1.92-1.82 (m, 2H), 1.45-1.33 (m, 2H), 1.29-1.17 (m, 9H), 1.12 (s, 6H).

### Example 20—Synthesis of N-((1r, 3r)-3-(3-chloro-4-cyanophenoxy)-2,2,4,4-tetramethylcyclobutyl)-5-(4-(2-(3-(4-(ethylsulfonamido)-2-(6-methyl-7-oxo-6,7-dihydro-1H-pyrrolo[2,3-c]pyridin-4-yl)phenoxy)phenoxy)ethoxy)piperidin-1-yl)pyrazine-2-carboxamide (I-71)

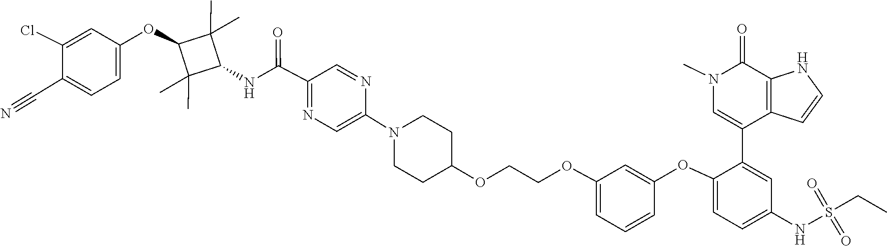 [SMILES: CCS(=O)(=O)Nc1ccc(Oc2cccc(OCCOC3CCN(c4cnc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1]

### 1.1 Synthesis of Compound 3

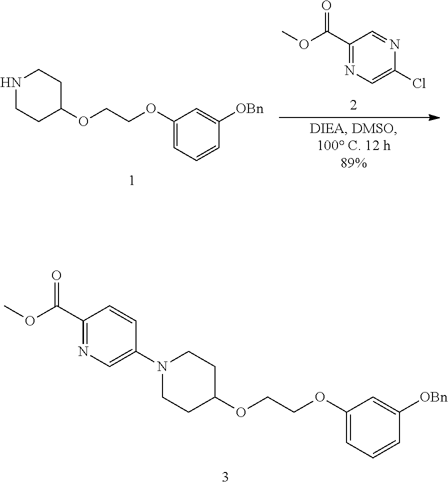 [SMILES: c1ccc(COc2cccc(OCCOC3CCNCC3)c2)cc1 ; COC(=O)c1cnc(Cl)cn1 ; COC(=O)c1ccc(N2CCC(OCCOc3cccc(OCc4ccccc4)c3)CC2)cn1]

To a solution of 4-[2-(3-benzyloxyphenoxy)ethoxy]piperidine (1.0 g, 2.9 μmol, 1.0 equiv, HCl) in DMSO (10 mL) was added DIEA (1.1 g, 8.6 μmol, 1.5 mL, 3.0 equiv) and methyl 5-chloropyrazine-2-carboxylate (597 mg, 3.4 μmol, 1.2 equiv). The mixture was stirred at 100° C. for 12 h. The reaction mixture was filtered and concentrated under reduced pressure to give a residue. The residue was purified by column chromatography (SiO₂, Petroleum ether/Ethyl acetate=10/1 to 5/1). Methyl 5-[4-[2-(3-benzyloxyphenoxy)ethoxy]-1-piperidyl]pyrazine-2-carboxylate (1.2 g, 890 yield) as a yellow solid. ¹H NMR (400 MHz, CDCl₃) δ=8.80 (d, J=1.2 Hz, 1H), 8.16 (d, J=1.2 Hz, 1H), 7.49-7.31 (m, 5H), 7.20 (t, J=8.0 Hz, 1H), 6.70-6.49 (m, 3H), 5.06 (s, 2H), 4.19-4.10 (m, 3H), 4.06-3.98 (m, 2H), 3.97 (s, 3H), 3.90-3.85 (m, 2H), 3.83-3.72 (m, 1H), 3.65-3.54 (m, 2H), 2.04-1.93 (m, 2H), 1.85-1.72 (m, 2H).

### 1.2 Synthesis of Compound 4

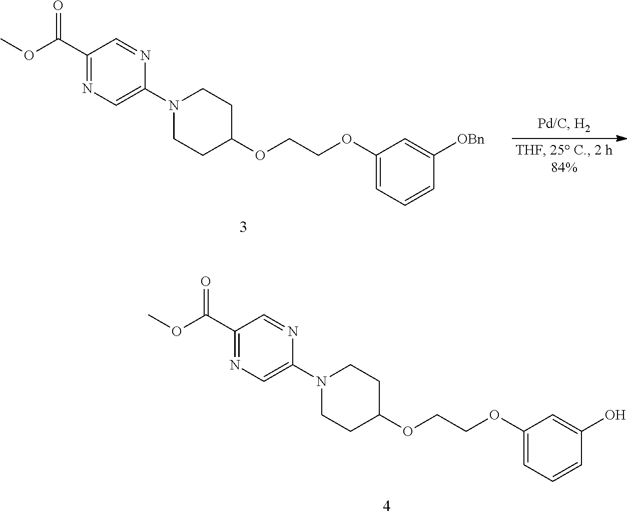 [SMILES: COC(=O)c1cnc(N2CCC(OCCOc3cccc(OCc4ccccc4)c3)CC2)cn1 ; COC(=O)c1cnc(N2CCC(OCCOc3cccc(O)c3)CC2)cn1]

To a solution of methyl 5-[4-[2-(3-benzyloxyphenoxy)ethoxy]-1-piperidyl]pyrazine-2-carboxylate (1.3 g, 2.8 μmol, 1.0 equiv) in THF (2 mL) was added Pd/C (130 mg, 10% purity) under N₂ atmosphere. The mixture was stirred under H₂ (15 Psi) at 25° C. for 2 h. The mixture was filtered and concentrated. The mixture was used for next step directly without further purification. 1HNMR (400 MHz, CDCl₃) δ 8.79 (d, J=1.2 Hz, 1H), 8.14 (d, J=1.2 Hz, 1H), 7.16-7.05 (m, 1H), 6.54-6.48 (m, 1H), 6.47-6.41 (m, 2H), 4.16-4.09 (m, 2H), 4.05-3.97 (m. 2H), 3.95 (s, 3H), 3.91-3.81 (m, 2H), 3.80-3.70 (m, 1H), 3.64-3.51 (m, 2H), 2.00-1.91 (m. 2H), 1.83-1.70 (m. 2H).

### 1.3 Synthesis of Compound 6

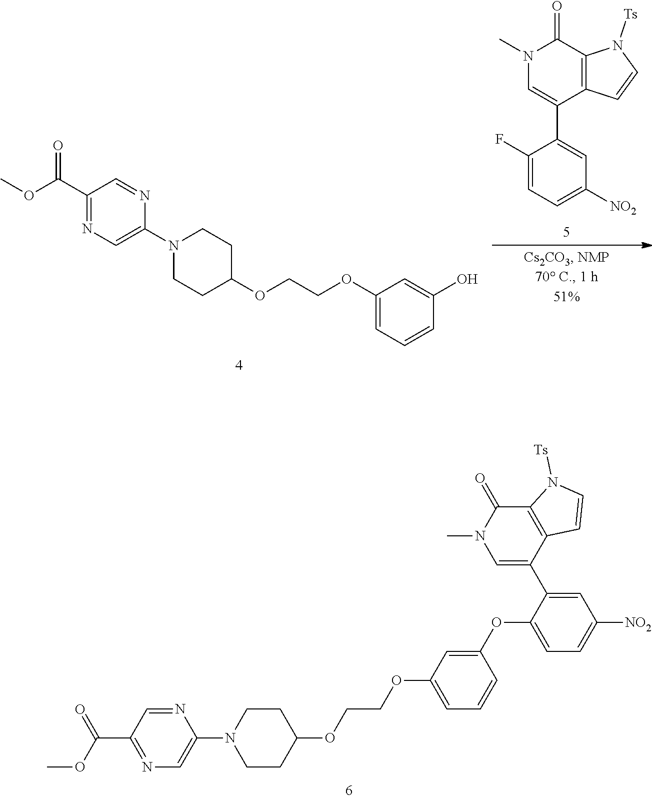 [SMILES: COC(=O)c1cnc(N2CCC(OCCOc3cccc(O)c3)CC2)cn1 ; Cc1ccc(S(=O)(=O)n2ccc3c(-c4cc([N+](=O)[O-])ccc4F)cn(C)c(=O)c32)cc1 ; COC(=O)c1cnc(N2CCC(OCCOc3cccc(Oc4ccc([N+](=O)[O-])cc4-c4cn(C)c(=O)c5c4ccn5S(=O)(=O)c4ccc(C)cc4)c3)CC2)cn1]

To a solution of methyl 5-[4-[2-(3-hydroxyphenoxy)ethoxy]-1-piperidyl]pyrazine-2-carboxylate (279 mg, 747 mol, 1.1 equiv) in NMP (2 mL) was added Cs₂CO₃ (442 mg, 1.3 mol, 2.0 equiv) and 4-(2-fluoro-5-nitro-phenyl)-6-methyl-1-(p-tolylsulfonyl)pyrrolo[2,3-c]pyridin-7-one (300 mg, 679 μmol, 1.0 equiv). The mixture was stirred at 70° C. for 1 h. Then the reaction mixture was filtered and concentrated. The residue was purified by prep-HPLC (column: Waters Xbridge C18 150 mm*50 mm*10 um; mobile phase: [water (NH₄HCO₃)-ACN]; B %: 47%-77%, 10 min) to give methyl 5-[4-[2-[3-[2-[6-methyl-7-oxo-1-(p-tolylsulfonyl)pyrrolo[2,3-c]pyridin-4-yl]-4-nitro-phenoxy]phenoxy]ethoxy]-1-piperidyl]pyrazine-2-carboxylate (277 mg, 51% yield).

### 1.4 Synthesis of Compound 7

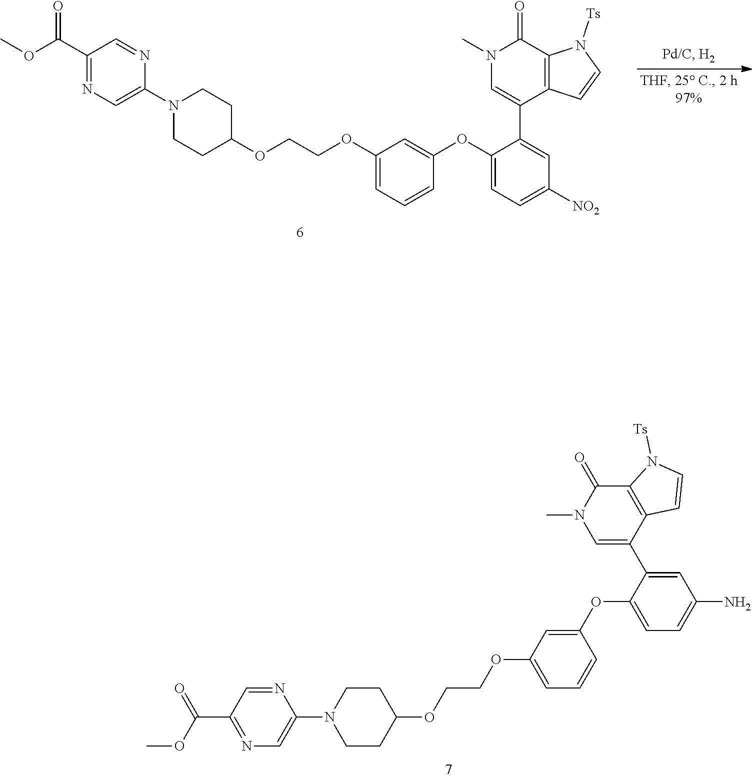 [SMILES: COC(=O)c1cnc(N2CCC(OCCOc3cccc(Oc4ccc([N+](=O)[O-])cc4-c4cn(C)c(=O)c5c4ccn5S(=O)(=O)c4ccc(C)cc4)c3)CC2)cn1 ; COC(=O)c1cnc(N2CCC(OCCOc3cccc(Oc4ccc(N)cc4-c4cn(C)c(=O)c5c4ccn5S(=O)(=O)c4ccc(C)cc4)c3)CC2)cn1]

To a solution of methyl 5-[4-[2-[3-[2-[6-methyl-7-oxo-1-(p-tolylsulfonyl) pyrrolo[2,3-c]pyridin-4-yl]-4-nitro-phenoxy]phenoxy]ethoxy]-1-piperidyl]pyrazine-2-carboxylate (277 mg, 348 mol, 1.0 equiv) in THF (4 mL) was added Pd/C (27 mg, 348 mol, 10% purity, 1.0 equiv) under N₂ atmosphere. The mixture was stirred under H₂ (15 Psi) at 25° C. for 2 h. The mixture was filtered and concentrated. The residue was used for next step directly without further purification.

### 1.5 Synthesis of Compound 8

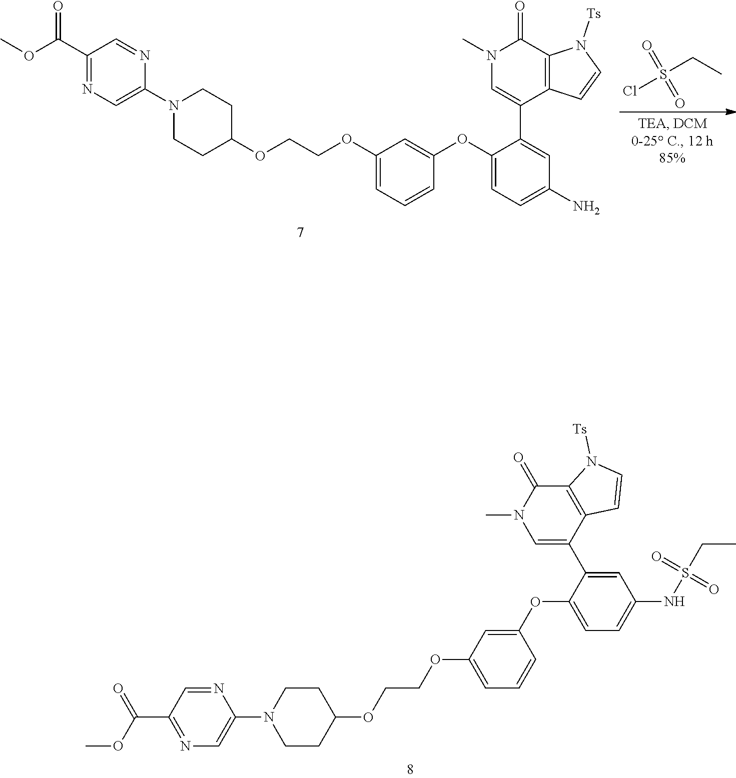

To a solution of methyl 5-[4-[2-[3-[4-amino-2-[6-methyl-7-oxo-1-(p-tolylsulfonyl) pyrrolo[2,3-c]pyridin-4-yl]phenoxy]phenoxy]ethoxy]-1-piperidyl]pyrazine-2-carboxylate (210 mg, 274 mol, 1.0 equiv) in DCM (3 mL) was added TEA (333 mg, 3.3 μmol, 12.0 equiv) and ethanesulfonyl chloride (71 mg, 549 gmol, 51 μL, 2.0 equiv). The mixture was stirred at 25° C. for 12 h. The reaction mixture was filtered and concentrated. The residue was used for next step directly without further purification.

### 1.6 Synthesis of Compound 9

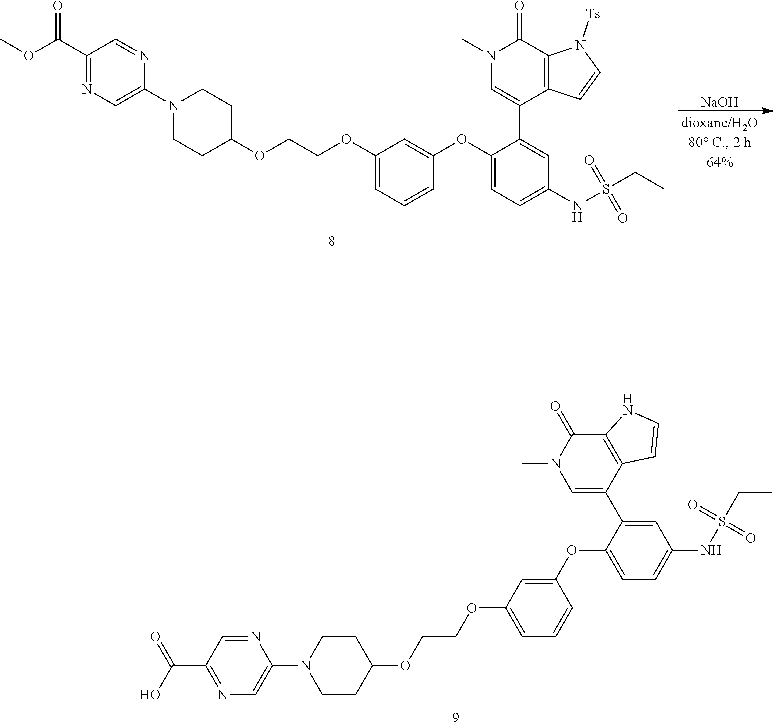 [SMILES: CCS(=O)(=O)Nc1ccc(Oc2cccc(OCCOC3CCN(c4cnc(C(=O)OC)cn4)CC3)c2)c(-c2cn(C)c(=O)c3c2ccn3S(=O)(=O)c2ccc(C)cc2)c1 ; "NaOH" ; CCS(=O)(=O)Nc1ccc(Oc2cccc(OCCOC3CCN(c4cnc(C(=O)O)cn4)CC3)c2)c(-c2cn(C)c(=O)c3[nH]ccc23)c1]

To a solution of methyl 5-[4-[2-[3-[4-(ethylsulfonylamino)-2-[6-methyl-7-oxo-1-(p-tolylsulfonyl)pyrrolo[2,3-c]pyridin-4-yl]phenoxy]phenoxy]ethoxy]-1-piperidyl]pyrazine-2-carboxylate (200 mg, 233 mol, 1.0 equiv) in dioxane (1 mL) and H₂O (1 mL) was added NaOH (242 mg, 6 mol, 26 equiv). The mixture was stirred at 80° C. for 2 h. The reaction mixture was filtered and concentrated. The residue was purified by prep-HPLC(column: 3_Phenomenex Luna C18 75 mm*30 mm*3 μm; mobile phase: [water(TFA)-ACN];B %: 38%-58%, 9 min) to give 5-[4-[2-[3-[4-(ethylsulfonylamino)-2-(6-methyl-7-oxo-1H-pyrrolo[2,3-c]pyridin-4-yl)phenoxy]phenoxy]ethoxy]-1-piperidyl]pyrazine-2-carboxylic acid (120 mg, 64% yield, TFA).

### 1.7 Synthesis of I-71

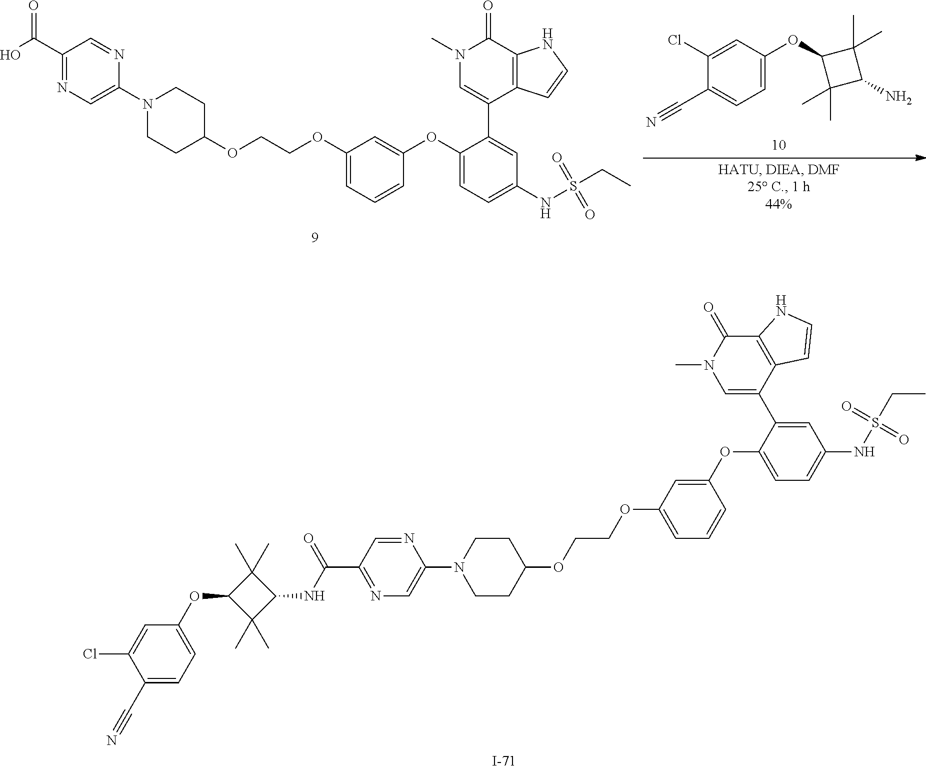

To a solution of 5-[4-[2-[3-[4-(ethylsulfonylamino)-2-(6-methyl-7-oxo-1H-pyrrolo[2,3-c]pyridin-4-yl)phenoxy]phenoxy]ethoxy]-1-piperidyl]pyrazine-2-carboxylic acid (60 mg. 74 mol, 1.0 equiv, TFA salt) and 4-(3-amino-2,2,4,4-tetramethyl-cyclobutoxy)-2-chloro-benzonitrile (20 mg, 74 μmol, 1.0 equiv) in DMF (1 mL) was added DIEA (48 mg, 373 μmol, 5.0 equiv) and HATU (36 mg, 97 μmol, 1.3 equiv). The mixture was stirred at 25° C. for 1.0 h. The mixture was filtered and concentrated. The residue was purified by prep-HPLC (column: Phenomenex C18 75 mm*30 mm*3 μm; mobile phase: [water(FA)-ACN]; B %: 55%-95%, 7 min) to give N-[3-(3-chloro-4-cyano-phenoxy)-2,2,4,4-tetramethyl-cyclobutyl]-5-[4-[2-[3-[4-(ethylsulfonylamino)-2-(6-methyl-7-oxo-1H-pyrrolo[2,3-c]pyridin-4-yl)phenoxy]phenoxy]ethoxy]-1-piperidyl]pyrazine-2-carboxamide (32 mg, 44% yield). 1H NMR (400 MHz, CDCl₃) δ 10.12-9.80 (m, 1H), 8.83 (s, 1H), 8.01 (s, 1H), 7.83-7.70 (m, 1H), 7.57 (d, J=8.8 Hz, 1H), 7.49-7.40 (m, 1H), 7.25-7.22 (m, 1H), 7.22-7.19 (m, 1H), 7.16-7.05 (m, 3H), 7.03-6.95 (m, 1H), 6.90-6.79 (m, 2H), 6.62-6.55 (m, 1H), 6.52-6.37 (m, 3H), 4.20-4.11 (m, 1H), 4.10-3.96 (m, 5H), 3.87-3.78 (m, 2H), 3.74-3.68 (m, 1H), 3.63 (s, 3H), 3.56-3.44 (m, 2H), 3.20 (q, J=7.6 Hz, 2H), 2.01-7.90 (m, 2H), 1.48-1.41 (m, 3H), 1.35-1.18 (m, 14H).

### Example 21—Synthesis of N-((1r, 3r)-3-(3-chloro-4-cyanophenoxy)-2,2,4,4-tetramethylcyclobutyl)-5-(4-(2-(3-(4-(ethylsulfonamido)-2-(6-methyl-7-oxo-6,7-dihydro-1H-pyrrolo[2,3-c]pyridin-4-yl)phenoxy)phenoxy)ethoxy)piperidin-1-yl)picolinamide (I-72)

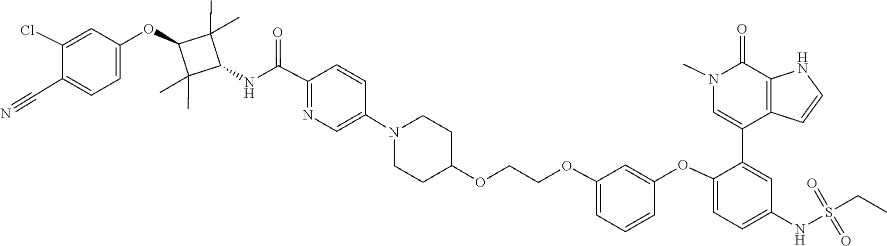 [SMILES: CCS(=O)(=O)Nc1ccc(Oc2cccc(OCCOC3CCN(c4ccc(C(=O)N[C@H]5C(C)(C)[C@H](Oc6ccc(C#N)c(Cl)c6)C5(C)C)nc4)CC3)c2)c(-c2cn(C)c(=O)c3[nH]ccc23)c1]

### 1.1 Synthesis of Compound 2

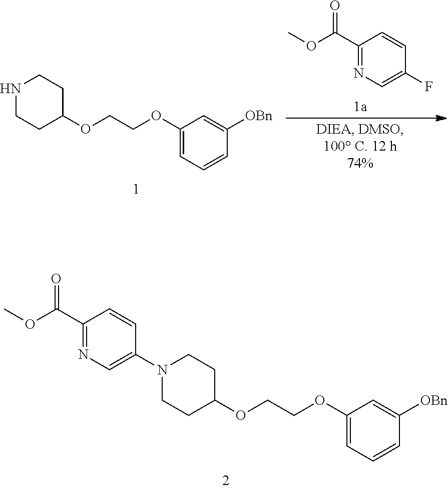 [SMILES: c1ccc(COc2cccc(OCCOC3CCNCC3)c2)cc1 ; COC(=O)c1ccc(F)cn1 ; COC(=O)c1ccc(N2CCC(OCCOc3cccc(OCc4ccccc4)c3)CC2)cn1]

To a solution of 4-[2-(3-benzyloxyphenoxy)ethoxy]piperidine (1.05 g, 3.2 μmol, 1.0 equiv) in DMSO (10 mL) was added DIEA (1.24 g, 9.6 mol, 3.0 equiv) and methyl 5-fluoropyridine-2-carboxylate (597 mg, 3.9 μmol, 1.2 equiv). The mixture was stirred at 100° C. for 12 h. The mixture was diluted with water (10 mL) and extracted with ethyl acetate (3×30 mL). The combined organic layers were washed with brine (90 mL), dried over sodium sulfate, filtered and concentrated. The residue was purified by column chromatography (SiO₂, Petroleum ether/Ethyl acetate=10/1 to 1/1) to give methyl 5-[4-[2-(3-benzyloxyphenoxy) ethoxy]-1-piperidyl]pyri dine-2-carboxylate (1.1 g, 74% yield) as a brown solid. ¹H NMR (400 MHz, CDCl₃): δ 8.36 (d, J=2.8 Hz, 1H), 7.99 (d, J=8.8 Hz, 1H), 7.48-7.30 (m, 5H), 7.22-7.11 (m, 2H), 6.63-6.51 (m, 3H), 5.05 (s, 2H), 4.13-4.09 (m, 2H), 3.97 (s, 3H), 3.88-3.82 (m, 2H), 3.72-3.62 (m, 3H), 3.28-3.17 (m, 2H), 2.04-1.95 (m, 2H), 1.85-1.73 (m, 2H).

### 1.2 Synthesis of Compound 3

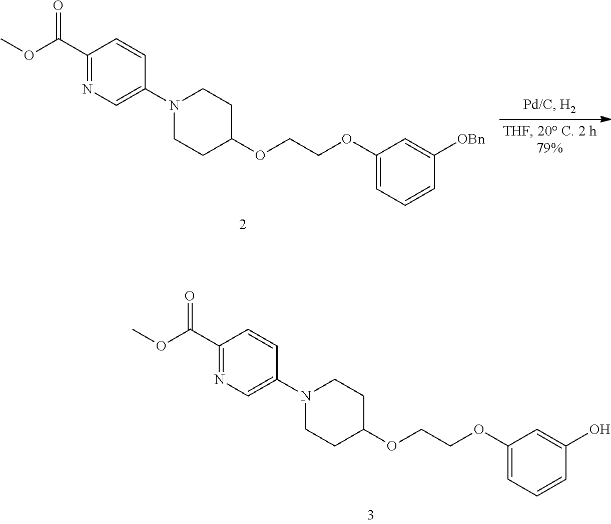 [SMILES: COC(=O)c1ccc(N2CCC(OCCOc3cccc(OCc4ccccc4)c3)CC2)cn1 ; COC(=O)c1ccc(N2CCC(OCCOc3cccc(O)c3)CC2)cn1]

To a solution of methyl 5-[4-[2-(3-benzyloxyphenoxy)ethoxy]-1-piperidyl]pyridine-2-carboxylate (1.1 g, 2.38 mol, 1.0 equiv) in THF (10 mL) was added Pd/C (110 mg, 10% purity) under N₂ atmosphere. The mixture was stirred under H₂ (15 psi) at 20° C. for 2 h. The mixture was filtered and concentrated. The residue was purified by column chromatography (SiO₂, Petroleum ether/Ethyl acetate=10/1 to 0/1) to give methyl 5-[4-[2-(3-hydroxyphenoxy)ethoxy]-1-piperidyl]pyridine-2-carboxylate (700 mg, 79% yield) as a yellow solid. ¹H NMR (400 MHz, CDCl₃): δ 8.32 (d, J=2.8 Hz, 1H), 7.99 (d, J=8.8 Hz, 1H), 7.21-7.04 (m, 2H), 6.53-6.41 (m, 3H), 4.18-4.05 (m, 2H), 3.94 (s, 3H), 3.89-3.79 (m, 2H), 3.73-3.55 (m, 3H), 3.28-3.13 (m, 2H), 2.05-1.91 (m, 2H), 1.85-1.68 (m, 2H).

### 1.3 Synthesis of Compound 4

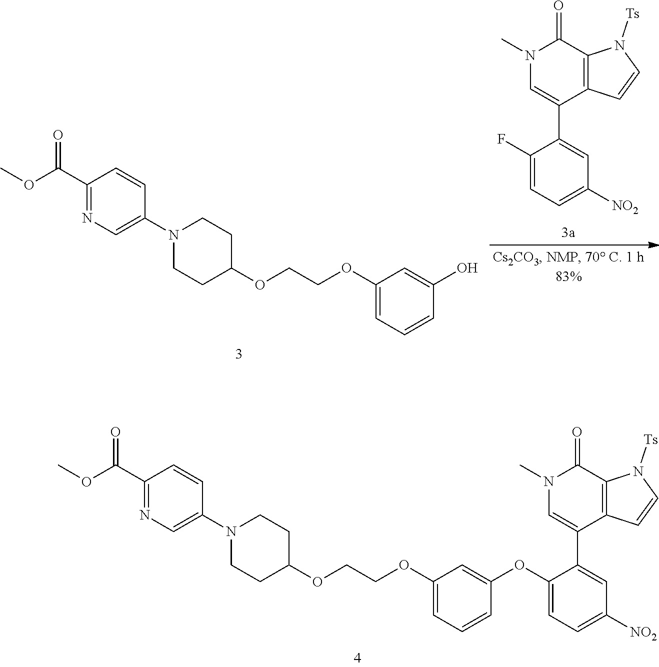 [SMILES: COC(=O)c1ccc(N2CCC(OCCOc3cccc(O)c3)CC2)cn1 ; Cc1ccc(S(=O)(=O)n2ccc3c(-c4cc([N+](=O)[O-])ccc4F)cn(C)c(=O)c32)cc1 ; COC(=O)c1ccc(N2CCC(OCCOc3cccc(Oc4ccc([N+](=O)[O-])cc4-c4cn(C)c(=O)c5c4ccn5S(=O)(=O)c4ccc(C)cc4)c3)CC2)cn1]

To a solution of methyl 5-[4-[2-(3-hydroxyphenoxy)ethoxy]-1-piperidyl]pyridine-2-carboxylate (278 mg, 746 mol, 1.1 eq) in NMP (δ mL) was added Cs₂CO₃ (442 mg, 1.36 μmol, 2.0 equiv) and 4-(2-fluoro-5-nitro-phenyl)-6-methyl-1-(p-tolylsulfonyl)pyrrolo[2,3-c]pyridin-7-one (300 mg, 679 mol, 1.0 equiv). The mixture was stirred at 70° C. for 1 h. The mixture was diluted with 5 mL water and extracted with Ethyl acetate (3×5 mL). The combined organic layers were washed with water (3×5 mL), dried over sodium sulfate, filtered and concentrated. The residue was purified by prep-HPLC (column: Waters Xbridge C18 150 mm*50 mm*10 μm; mobile phase: [water(NH₄HCO₃)-ACN]; B %: 36%-66%, 10 min) to give methyl 5-[4-[2-[3-[2-[6-methyl-7-oxo-1-(p-tolylsulfonyl)pyrrolo[2,3-c]pyridin-4-yl]-4-nitro-phenoxy]phenoxy]ethoxy]-1-piperidyl]pyridine-2-carboxylate (450 mg, 83% yield) as a yellow oil. ¹H NMR (400 MHz, CDCl₃): δ 8.36-8.33 (m, 1H), 8.28 (d, J=2.8 Hz, 1H), 8.20-8.13 (m, 1H), 8.08-8.02 (m, 2H), 8.01-7.97 (m, 1H), 7.93 (d, J=3.6 Hz, 1H), 7.35-7.30 (m, 3H), 7.19-7.13 (m, 2H), 7.00 (d, J=8.8 Hz, 1H), 6.80-6.75 (m, 1H), 6.59-6.53 (m, 2H), 6.40 (d, J=3.2 Hz, 1H), 4.13-4.08 (m, 2H), 3.97 (s, 3H), 3.87-3.82 (m, 2H), 3.70-3.62 (m, 4H), 3.57 (s, 3H), 3.25-3.17 (m, 2H), 2.42 (s, 3H), 2.00-1.97 (m, 1H), 1.82-1.73 (m, 2H)

### 1.4 Synthesis of Compound 5

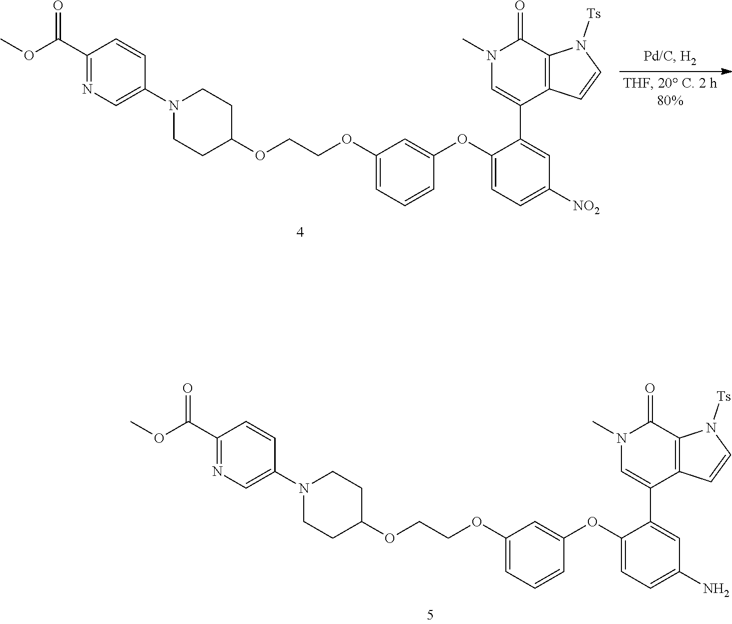 [SMILES: COC(=O)c1ccc(N2CCC(OCCOc3cccc(Oc4ccc([N+](=O)[O-])cc4-c4cn(C)c(=O)c5c4ccn5S(=O)(=O)c4ccc(C)cc4)c3)CC2)cn1 ; COC(=O)c1ccc(N2CCC(OCCOc3cccc(Oc4ccc(N)cc4-c4cn(C)c(=O)c5c4ccn5S(=O)(=O)c4ccc(C)cc4)c3)CC2)cn1]

To a solution of methyl 5-[4-[2-[3-[2-[6-methyl-7-oxo-1-(p-tolylsulfonyl)pyrrolo [2,3-c]pyridin-4-yl]-4-nitro-phenoxy]phenoxy]ethoxy]-1-piperidyl]pyridine-2-carboxylate (450 mg, 566 mol, 1.0 equiv) in THF (5 mL) was added Pd/C (50 mg, 10% purity) under N₂ atmosphere. The mixture was stirred under H₂ (15 psi) at 20° C. for 2 h. The mixture was filtered and concentrated to give methyl 5-[4-[2-[3-[4-amino-2-[6-methyl-7-oxo-1-(p-tolylsulfonyl)pyrrolo[2,3-c]pyridin-4-yl]phenoxy]phenoxy]ethoxy]-1-piperidyl]pyridine-2-carboxylate (350 mg, 80% yield) as a yellow solid.

### 1.5 Synthesis of Compound 6

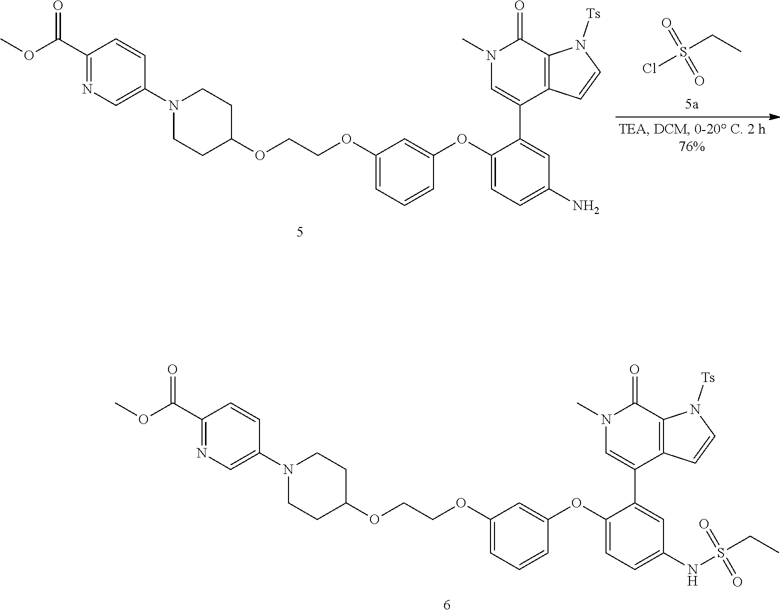

To a solution of methyl 5-[4-[2-[3-[4-amino-2-[6-methyl-7-oxo-1-(p-tolylsulfonyl) pyrrolo[2,3-c]pyridin-4-yl]phenoxy]phenoxy]ethoxy]-1-piperidyl]pyridine-2-carboxylate (350 mg, 458 mol, 1.0 equiv) in DCM (4 mL) was added TEA (556 mg, 5.5 mol, 12 equiv) and ethanesulfonyl chloride (353 mg, 2.75 μmol, 6.0 equiv) at 0° C. The mixture was stirred at 20° C. for 2 h. The solution was concentrated to give methyl 5-[4-[2-[3-[4-(ethylsulfonyl-amino)-2-[6-methyl-7-oxo-1-(p-tolylsulfonyl)pyrrolo[2,3-c]pyridin-4-yl]phenoxy]phenoxy]ethoxy]-1-piperidyl]pyridine-2-carboxylate (300 mg, 76% yield).

### 1.6 Synthesis of Compound 7

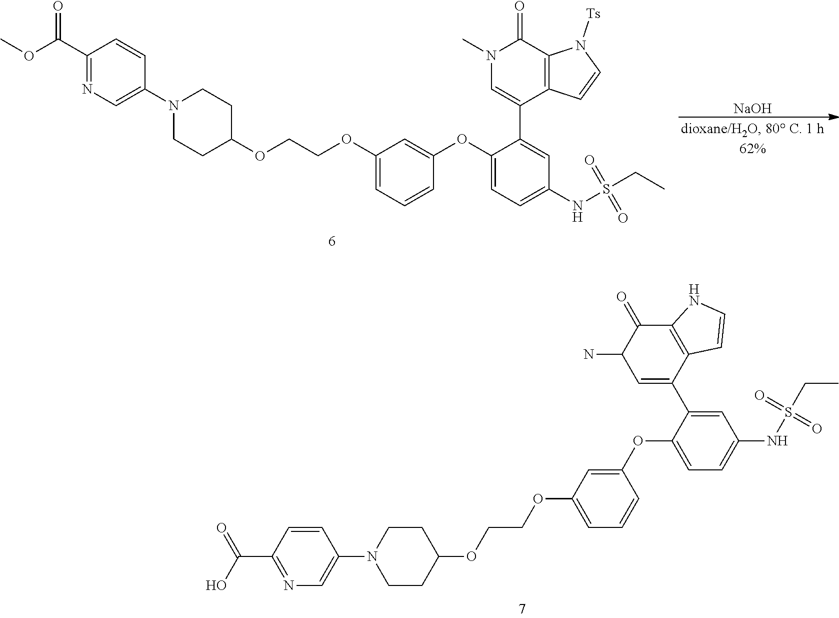 [SMILES: CCS(=O)(=O)Nc1ccc(Oc2cccc(OCCOC3CCN(c4ccc(C(=O)OC)nc4)CC3)c2)c(-c2cn(C)c(=O)c3c2ccn3S(=O)(=O)c2ccc(C)cc2)c1 ; "NaOH" ; CCS(=O)(=O)Nc1ccc(Oc2cccc(OCCOC3CCN(c4ccc(C(=O)O)nc4)CC3)c2)c(C2=CC(N)C(=O)c3[nH]ccc32)c1]

To a solution of methyl 5-[4-[2-[3-[4-(ethylsulfonylamino)-2-[6-methyl-7-oxo-1-(p-tolylsulfonyl)pyrrolo[2,3-c]pyridin-4-yl]phenoxy]phenoxy]ethoxy]-1-piperidyl]pyridine-2-carboxylate (300 mg, 350 mol, 1.0 equiv) in dioxane (5 mL) and H₂O (2 mL) was added NaOH (840 mg, 21 μmol, 60 equiv). The mixture was stirred at 80° C. for 1 h and concentrated. The residue was purified by prep-HPLC (column: 3 Phenomenex Luna C18 75 mm*30 mm*3 μm; mobile phase: [water(TFA)-ACN]; B %: 30%-50%, 9 min) to give 5-[4-[2-[3-[4-(ethylsulfonylamino)-2-(6-methyl-7-oxo-1H-pyrrolo[2,3-c]pyridin-4-yl)phenoxy]phenoxy]ethoxy]-1-piperidyl]pyridine-2-carboxylic acid (150 mg, 62% yield). ¹H NMR (400 MHz, MeOD): δ 12.05 (s, 1H), 9.83 (s, 1H), 8.34 (d, J=2.8 Hz, 1H), 7.89 (d, J=8.8 Hz, 1H), 7.49-7.38 (m, 2H), 7.33-7.25 (m, 2H), 7.25-7.19 (m, 1H), 7.16-7.10 (m, 1H), 7.06 (d, J=8.8 Hz, 1H), 6.61-6.54 (m, 1H), 6.44-6.36 (m, 2H), 6.28-6.24 (m, 1H), 4.02-3.94 (m, 2H), 3.76-3.66 (m, 4H), 3.21-3.08 (m, 4H), 1.95-1.85 (m, 2H), 1.78-1.73 (m, 4H), 1.57-1.43 (m, 2H), 1.27-1.18 (m, 3H).

### 1.7 Synthesis of I-72

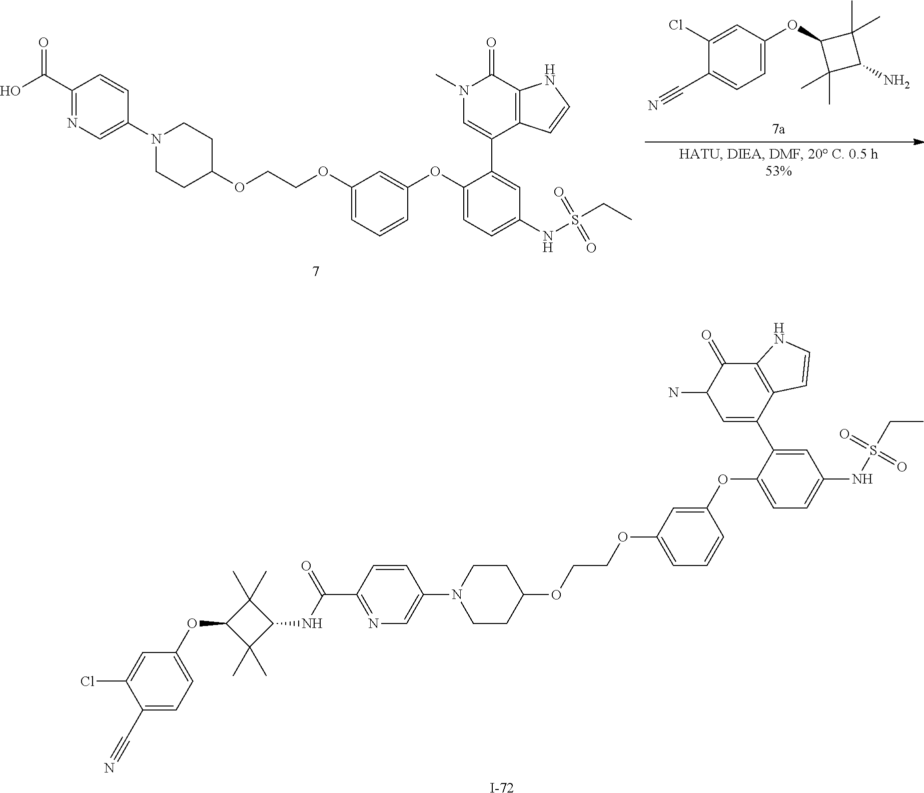

To a solution of 5-[4-[2-[3-[4-(ethylsulfonylamino)-2-(6-methyl-7-oxo-1H-pyrrolo[2,3-c]pyridin-4-yl)phenoxy]phenoxy]ethoxy]-1-piperidyl]pyridine-2-carboxylic acid (50 mg, 62 μmol, 1.0 equiv) in DMF (1 mL) was added DIEA (24 mg, 187 μmol, 3.0 equiv) and 4-(3-amino-2,2,4,4-tetramethyl-cyclobutoxy)-2-chloro-benzonitrile (25 mg, 63 μmol, 1.0 equiv) and HATU (30 mg, 81 μmol, 1.3 equiv). The mixture was stirred at 20° C. for 0.5 h and concentrated. The residue was purified by prep-HPLC (column: Phenomenex C18 75 mm*30 mm*3 μm; mobile phase: [water(FA)-ACN]; B %: 58%-88%, 7 min) to give N-[3-(3-chloro-4-cyano-phenoxy)-2,2,4,4-tetramethyl-cyclobutyl]-5-[4-[2-[3-[4-(ethylsulfonylamino)-2-(6-methyl-7-oxo-1H-pyrrolo[2,3-c]pyridin-4-yl)phenoxy]phenoxy]ethoxy]-1-piperidyl]pyridine-2-carboxamide (31 mg, 53% yield). ¹H NMR (400 MHz, DMSO-d₆): δ 12.06 (s, 1H), 9.83 (s, 1H), 8.34 (d, J=2.4 Hz, 1H), 8.09 (d, J=9.2 Hz, 1H), 7.90 (d, J=8.8 Hz, 1H), 7.83 (d, J=8.8 Hz, 1H), 7.45-7.38 (m, 2H), 7.30-7.27 (m, 2H), 7.26-7.20 (m, 2H), 7.16-7.10 (m, 1H), 7.08-7.00 (m, 2H), 6.62-6.54 (m, 1H), 6.45-6.34 (m, 2H), 6.28-6.24 (m, 1H), 4.43 (s, 1H), 4.01-3.91 (m, 3H), 3.75-3.64 (m, 4H), 3.62-3.55 (m, 1H), 3.49 (s, 3H), 3.16-3.06 (m, 4H), 1.95-1.85 (m, 2H), 1.55-1.44 (m, 2H), 1.25-1.21 (m, 3H), 1.19 (s, 6H), 1.12 (s, 6H).

### Example 22—Synthesis of N-((1r, 3r)-3-(3-chloro-4-cyanophenoxy)-2,2,4,4-tetramethylcyclobutyl)-5-(4-(2-(3-(4-(ethylsulfonamido)-2-(6-methyl-7-oxo-6,7-dihydro-1H-pyrrolo[2,3-c]pyridin-4-yl)phenoxy)phenoxy)ethoxy)piperidin-1-yl)pyrimidine-2-carboxamide (I-73)

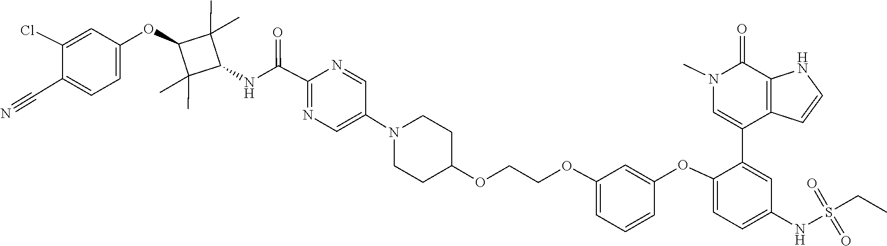 [SMILES: CCS(=O)(=O)Nc1ccc(Oc2cccc(OCCOC3CCN(c4cnc(C(=O)N[C@H]5C(C)(C)[C@H](Oc6ccc(C#N)c(Cl)c6)C5(C)C)nc4)CC3)c2)c(-c2cn(C)c(=O)c3[nH]ccc23)c1]

### 1.1 Synthesis of Compound 2

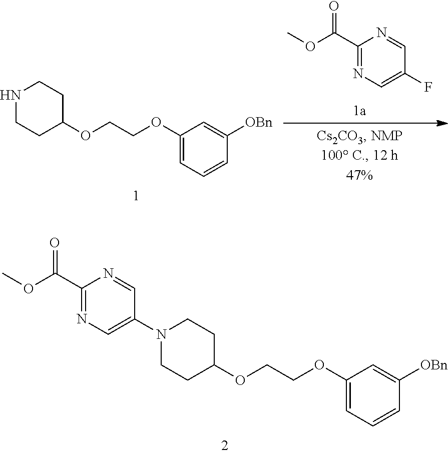 [SMILES: c1ccc(COc2cccc(OCCOC3CCNCC3)c2)cc1 ; COC(=O)c1ncc(F)cn1 ; COC(=O)c1ncc(N2CCC(OCCOc3cccc(OCc4ccccc4)c3)CC2)cn1]

To a solution of 4-[2-(3-benzyloxyphenoxy)ethoxy]piperidine (1 g, 3 μmol, 1.0 equiv, HCl) in NMP (10 mL) was added Cs₂CO₃ (2.7 g, 8 μmol, 3.0 equiv) and methyl 5-fluoropyrimidine-2-carboxylate (558 mg, 3.6 mol, 1.3 equiv). The mixture was stirred at 100° C. for 12 h and concentrated. The residue was purified by prep-HPLC (column: Waters Xbridge Cis 150 mm*50 mm*10 μm; mobile phase: [water(NH₄HCO₃)-ACN]; B %: 44%-74%, 10 min) to give methyl 5-[4-[2-(3-benzyloxyphenoxy)ethoxy]-1-piperidyl]pyrimidine-2-carboxylate (600 mg, 47% yield). ¹H NMR (400 MHz, CDCl₃): δ 8.43 (s, 2H), 7.36-7.45 (m, 4H), 7.30-7.36 (m, 1H), 7.18 (t, J=8.2 Hz, 1H), 6.52-6.62 (m, 3H), 5.04 (s, 2H), 4.11-4.14 (m, 2H), 4.03 (s, 3H), 3.84-3.87 (m, 2H), 3.70-3.75 (m, 1H), 3.62-3.68 (m, 2H), 3.26-3.34 (m, 2H), 1.96-2.04 (m, 2H), 1.79-1.88 (m, 2H).

### 1.2 Synthesis of Compound 3

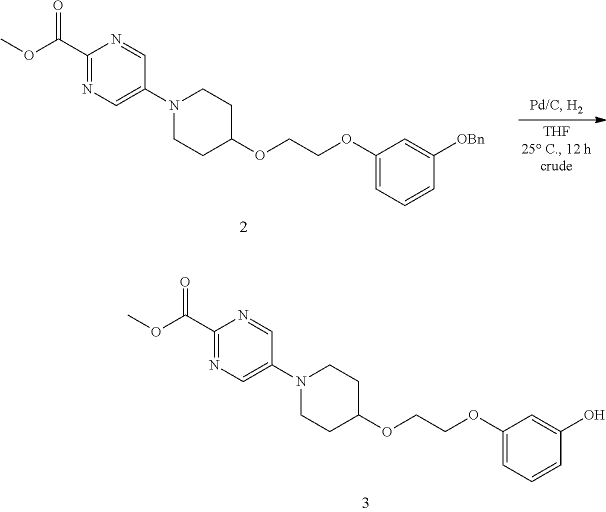 [SMILES: COC(=O)c1ncc(N2CCC(OCCOc3cccc(OCc4ccccc4)c3)CC2)cn1 ; COC(=O)c1ncc(N2CCC(OCCOc3cccc(O)c3)CC2)cn1]

To a solution of methyl 5-[4-[2-(3-benzyloxyphenoxy)ethoxy]-1-piperidyl]pyrimidine-2-carboxylate (700 mg, 1.5 μmol, 1.0 equiv) in THF (8 mL) was added Pd/C (70 mg, 10% purity) under N₂ atmosphere. The mixture was stirred under H₂ (15 Psi) at 25° C. for 12 h. The mixture was filtered and concentrated to give a methyl 5-[4-[2-(3-hydroxyphenoxy) ethoxy]-1-piperidyl]pyrimidine-2-carboxylate (400 mg, crude). ¹H NMR (400 MHz, DMSO-d₆): δ 8.56 (s, 1H), 7.03 (s, 1H), 6.34-6.37 (m, 2H), 6.31-6.33 (m, 2H), 4.01-4.03 (m, 2H), 3.82 (s, 3H), 3.75-3.78 (m, 4H), 3.62-3.69 (m, 2H), 3.22-3.25 (m, 2H), 3.21 (s, 1H), 1.93-1.97 (m, 2H).

### 1.3 Synthesis of Compound 4

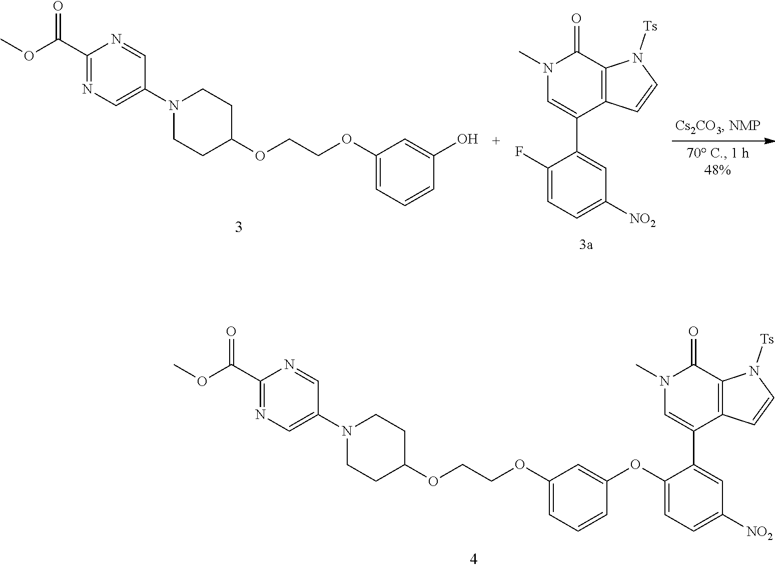 [SMILES: COC(=O)c1ncc(N2CCC(OCCOc3cccc(O)c3)CC2)cn1 ; Cc1ccc(S(=O)(=O)n2ccc3c(-c4cc([N+](=O)[O-])ccc4F)cn(C)c(=O)c32)cc1 ; COC(=O)c1ncc(N2CCC(OCCOc3cccc(Oc4ccc([N+](=O)[O-])cc4-c4cn(C)c(=O)c5c4ccn5S(=O)(=O)c4ccc(C)cc4)c3)CC2)cn1]

To a solution of methyl 5-[4-[2-(3-hydroxyphenoxy)ethoxy]-1-piperidyl]pyrimidine-2-carboxylate (279 mg, 748 mol, 1.1 equiv) in NMP (3 mL) was added Cs₂CO₃ (443 mg, 1.4 μmol, 2.0 equiv) and 4-(2-fluoro-5-nitro-phenyl)-6-methyl-1-(p-tolylsulfonyl) pyrrolo[2,3-c]pyridin-7-one (300 mg, 679 mol, 1.0 equiv). The mixture was stirred at 70° C. for 1 h and concentrated. The residue was purified by prep-HPLC (column: Waters Xbridge C18 150 mm*50 mm*10 μm; mobile phase: [water(NH₄HCO₃)-ACN]; B %: 44%-74%, 10 min) to give methyl 5-[4-[2-[3-[2-[6-methyl-7-oxo-1-(p-tolylsulfonyl)pyrrolo [2,3-c]pyridin-4-yl]-4-nitro-phenoxy]phenoxy]ethoxy]-1-piperidyl]pyrimidine-2-carboxylate (260 mg, 48% yield).

¹H NMR (400 MHz, CDCl₃): δ 8.43 (s, 2H), 8.28 (d, J=2.8 Hz, 1H), 8.15-8.18 (m, 1H), 8.05 (d, J=8.4 Hz, 2H), 7.92 (d, J=3.6 Hz, 1H), 7.33 (d, J=8.2 Hz, 3H), 7.17 (s, 1H), 7.01 (d, J=9.2 Hz, 1H), 6.75-6.78 (m, 1H), 6.53-6.58 (m, 2H), 6.40 (d, J=3.6 Hz, 1H), 4.09-4.11 (m, 2H), 4.03 (s, 3H), 3.84-3.86 (m, 2H), 3.70-3.73 (m, 1H), 3.62-3.65 (m, 2H), 3.57 (s, 3H), 3.26-3.32 (m, 2H), 2.42 (s, 3H), 1.98-2.05 (m, 2H), 1.80-1.86 (m, 2H).

### 1.4 Synthesis of Compound 5

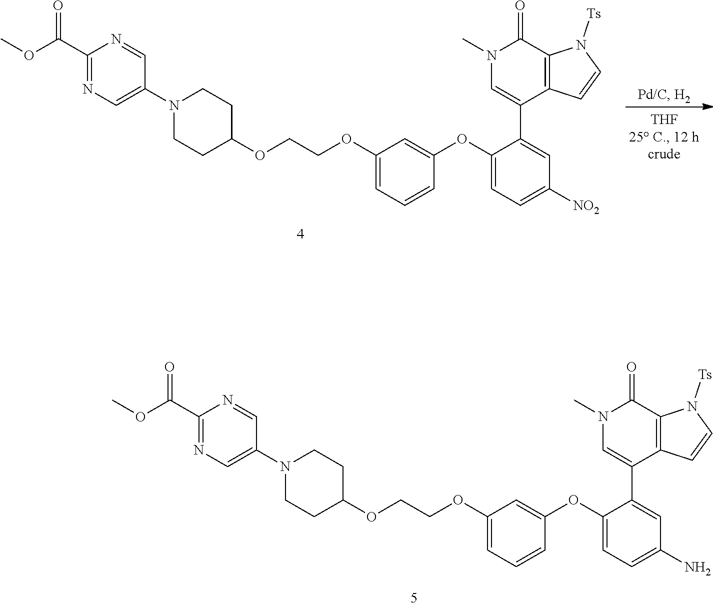 [SMILES: COC(=O)c1ncc(N2CCC(OCCOc3cccc(Oc4ccc([N+](=O)[O-])cc4-c4cn(C)c(=O)c5c4ccn5S(=O)(=O)c4ccc(C)cc4)c3)CC2)cn1 ; COC(=O)c1ncc(N2CCC(OCCOc3cccc(Oc4ccc(N)cc4-c4cn(C)c(=O)c5c4ccn5S(=O)(=O)c4ccc(C)cc4)c3)CC2)cn1]

To a solution of methyl 5-[4-[2-[3-[2-[6-methyl-7-oxo-1-(p-tolylsulfonyl) pyrrolo[2,3-c]pyridin-4-yl]-4-nitro-phenoxy]phenoxy]ethoxy]-1-piperidyl]pyrimidine-2-carboxylate (270 mg, 340 mol, 1.0 equiv) in THF (3 mL) was added Pd/C (30 mg, 10% purity) under N₂ atmosphere. The mixture was stirred under H₂ (15 Psi) at 25° C. for 12 h. The reaction mixture was filtered and concentrated to give methyl 5-[4-[2-[3-[4-amino-2-[6-methyl-7-oxo-1-(p-tolylsulfonyl)pyrrolo[2,3-c]pyridin-4-yl]phenoxy]phenoxy]ethoxy]-1-piperidyl]pyrimidine-2-carboxylate (200 mg, crude). ¹H NMR (400 MHz, CDCl₃): δ 8.43 (s, 2H), 8.00 (d, J=7.8 Hz, 2H), 7.86 (d, J=3.2 Hz, 1H), 7.30 (d, J=7.8 Hz, 3H), 6.92-7.06 (m, 4H), 6.67-6.71 (m, 2H), 6.45-6.50 (m, 2H), 6.26-6.33 (m, 2H), 4.03 (s, 3H), 4.00-4.01 (m, 2H), 3.78-3.81 (m, 2H), 3.61-3.72 (m, 4H), 3.43 (s, 3H), 3.24-3.33 (m, 2H), 2.41 (s, 3H), 1.93-2.04 (m, 2H), 1.78-1.83 (m, 1H).

### 1.5 Synthesis of Compound 6

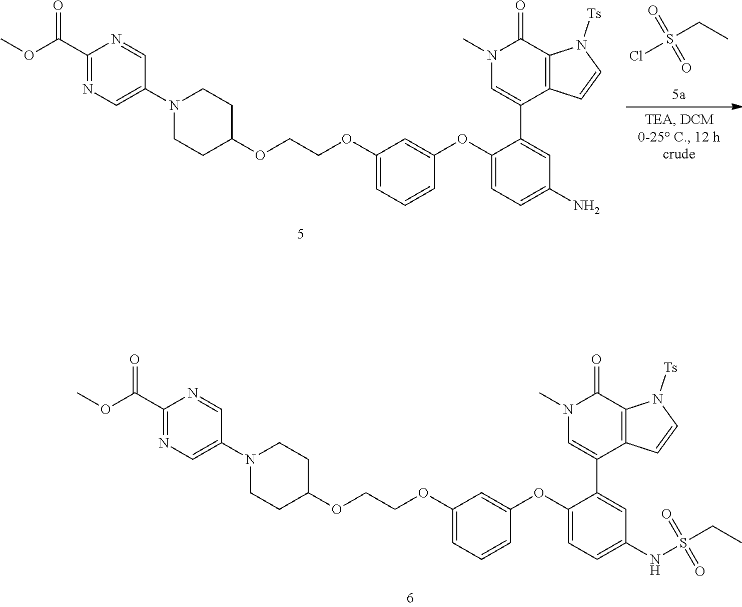

To a solution of methyl 5-[4-[2-[3-[4-amino-2-[6-methyl-7-oxo-1-(p-tolylsulfonyl) pyrrolo[2,3-c]pyridin-4-yl]phenoxy]phenoxy]ethoxy]-1-piperidyl]pyrimidine-2-carboxylate (200 mg, 261 mol, 1.0 equiv) in DCM (3 mL) was added TEA (318 mg, 3 mol, 12.0 equiv) and ethanesulfonyl chloride (101 mg, 784 μmol, 3.0 equiv) at 0° C. The mixture was stirred at 25° C. for 12 h and concentrated to give a crude product.

### 1.6 Synthesis of Compound 7

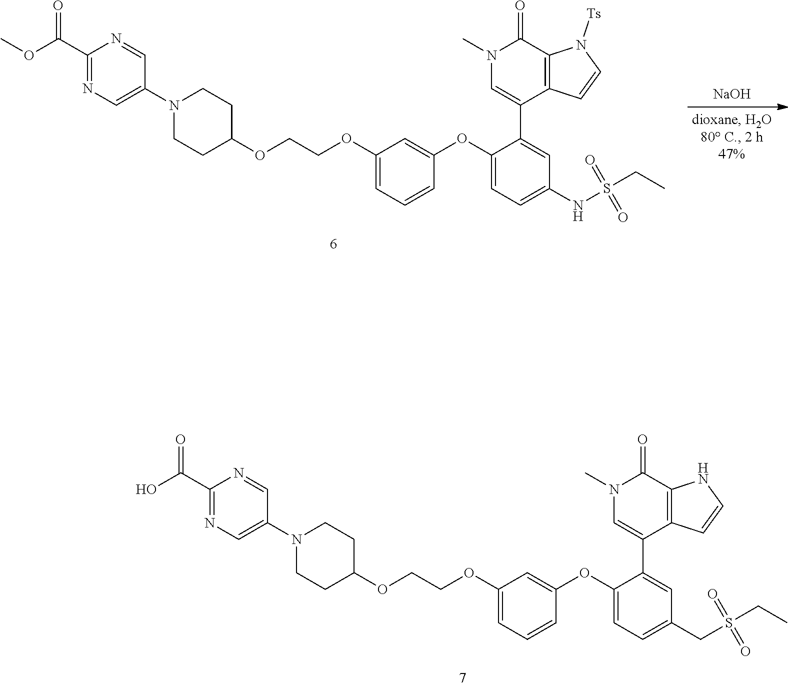 [SMILES: CCS(=O)(=O)Nc1ccc(Oc2cccc(OCCOC3CCN(c4cnc(C(=O)OC)nc4)CC3)c2)c(-c2cn(C)c(=O)c3c2ccn3S(=O)(=O)c2ccc(C)cc2)c1 ; "NaOH" ; CCS(=O)(=O)Cc1ccc(Oc2cccc(OCCOC3CCN(c4cnc(C(=O)O)nc4)CC3)c2)c(-c2cn(C)c(=O)c3[nH]ccc23)c1]

To a solution of methyl 5-[4-[2-[3-[4-(ethylsulfonylamino)-2-[6-methyl-7-oxo-1-(p-tolylsulfonyl)pyrrolo[2,3-c]pyridin-4-yl]phenoxy]phenoxy]ethoxy]-1-piperidyl]pyrimidine-2-carboxylate(180 mg, 210 mol, 1.0 equiv) in dioxane (0.5 mL) was added NaOH (84 mg, 2.1 μmol, 10 equiv) and H₂O(0.5 mL). The mixture was stirred at 80° C. for 2 h and concentrated. The residue was purified by prep-HPLC (column: 3_Phenomenex Luna C18 75 mm*30 mm*3 μm; mobile phase: [water(TFA)-ACN]; B %: 34%-54%, 9 min) to give 5-[4-[2-[3-[4-(ethylsulfonylamino)-2-(6-methyl-7-oxo-1H-pyrrolo[2,3-c]pyridin-4-yl)phenoxy]phenoxy]ethoxy]-1-piperidyl]pyrimidine-2-carboxylic acid (84 mg, 47% yield). ¹H NMR (400 MHz, DMSO-d₆): δ 12.04 (s, 1H), 9.81 (s, 1H), 8.54 (s, 2H), 7.40 (d, J=2.4 Hz, 1H), 7.26-7.30 (m, 2H), 7.21-7.24 (m, 1H), 7.13 (t, J=8.4 Hz, 1H), 7.06 (d, J=8.6 Hz, 1H), 6.56-6.60 (m, 1H), 6.38-6.42 (m, 2H), 6.25-6.26 (m, 1H), 3.97-4.00 (m, 2H), 3.72-3.76 (m, 2H), 3.58-3.62 (m, 4H), 3.49 (s, 3H), 3.17-3.20 (m, 1H), 3.09-3.14 (m, 2H), 1.87-1.95 (m, 2H), 1.45-1.56 (m, 2H), 1.21-1.25 (m, 3H).

### 1.7 Synthesis of I-73

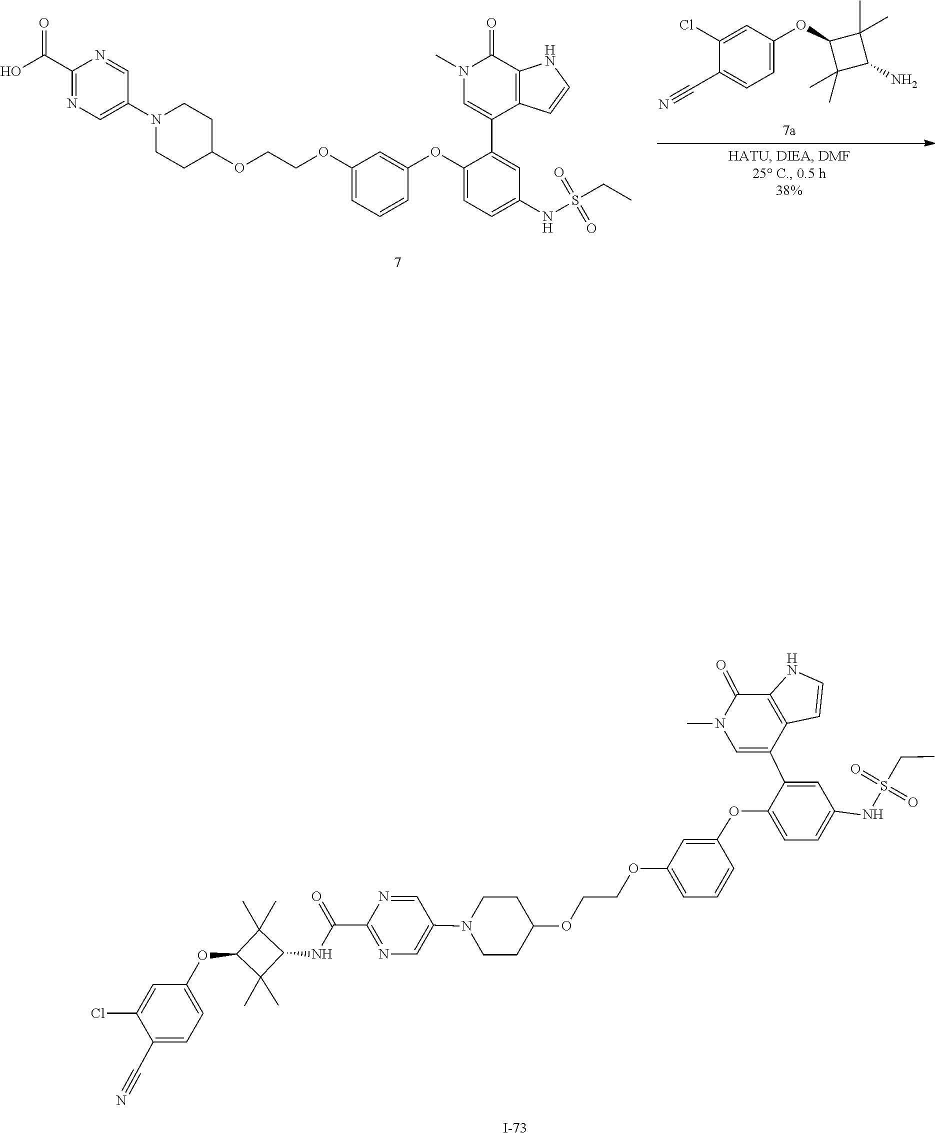

To a solution of 5-[4-[2-[3-[4-(ethylsulfonylamino)-2-(6-methyl-7-oxo-1H-pyrrolo[2,3-c]pyridin-4-yl)phenoxy]phenoxy]ethoxy]-1-piperidyl]pyrimidine-2-carboxylic acid (40 mg, 50 mol, 1.0 equiv) in DMF (1 mL) was added DIEA (32 mg, 249 μmol, 5.0 equiv), 4-(3-amino-2,2,4,4-tetramethyl-cyclobutoxy)-2-chloro-benzonitrile (14 mg, 35 mol, 0.71 equiv, TFA salt) and HATU (25 mg, 65 mol, 1.3 equiv). The mixture was stirred at 25° C. for 0.5 h and concentrated. The residue was purified by prep-HPLC (column: Phenomenex C18 75 mm*30 mm*3 μm; mobile phase: [water(FA)-ACN]; B %: 58%-88%, 7 min) to give N-[3-(3-chloro-4-cyano-phenoxy)-2,2,4,4-tetramethyl-cyclobutyl]-5-[4-[2-[3-[4-(ethylsulfonylamino)-2-(6-methyl-7-oxo-1H-pyrrolo[2,3-c]pyridin-4-yl)phenoxy]phenoxy]ethoxy]-1-piperidyl]pyrimidine-2-carboxamide (18 mg, 38% yield). ¹H NMR (400 MHz, CDCl₃): δ 10.65 (s, 1H), 8.36 (s, 2H), 8.22-8.31 (m, 1H), 8.08 (d, J=8.8 Hz, 1H), 7.54-7.58 (m, 2H), 7.30-7.33 (m, J=8.6, 2.69 Hz, 1H), 7.25-7.27 (m, 1H), 7.11 (t, J=8.2 Hz, 1H), 7.03-7.07 (m, 2H), 6.97 (d, J=2.4 Hz, 1H), 6.80-6.83 (m, 1H), 6.52-6.55 (m, 1H), 6.45-6.47 (m, 1H), 6.39-6.40 (m, 1H), 6.35-6.36 (m, 1H), 4.18-4.20 (m, 1H), 4.08 (s, 1H), 3.98-4.02 (m, 2H), 3.76-3.79 (m, 2H), 3.64-3.69 (m, 1H), 3.58 (s, 3H), 3.52-3.58 (m, 2H), 3.19-3.27 (m, 4H), 1.92-1.99 (m, 2H), 1.74-1.81 (m, 2H), 1.41-1.45 (m, 3H), 1.27 (s, 6H), 1.21 (s, 6H).

### Example 23—Synthesis of N-((1r, 3r)-3-(3-chloro-4-cyanophenoxy)-2,2,4,4-tetramethylcyclobutyl)-2-(4-(1-(3-(2-(6-methyl-7-oxo-6,7-dihydro-1H-pyrrolo[2,3-c]pyridin-4-yl)-4-(N-methylcyclopropanesulfonamido)phenoxy)phenyl)azetidin-3-yl)piperazin-1-yl)pyrimidine-5-carboxamide (I-88)

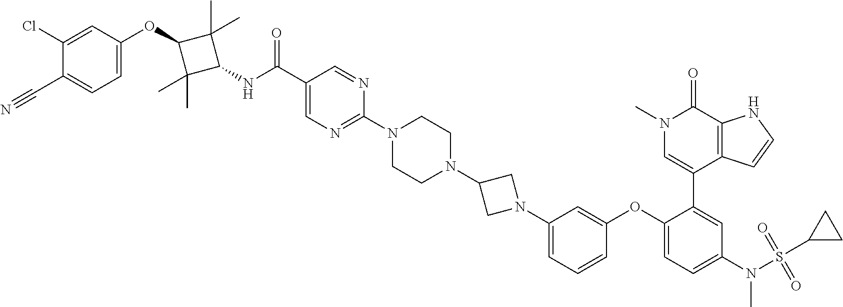 [SMILES: CN(c1ccc(Oc2cccc(N3CC(N4CCN(c5ncc(C(=O)N[C@H]6C(C)(C)[C@H](Oc7ccc(C#N)c(Cl)c7)C6(C)C)cn5)CC4)C3)c2)c(-c2cn(C)c(=O)c3[nH]ccc23)c1)S(=O)(=O)C1CC1]

### 1.1 Synthesis of Compound 4

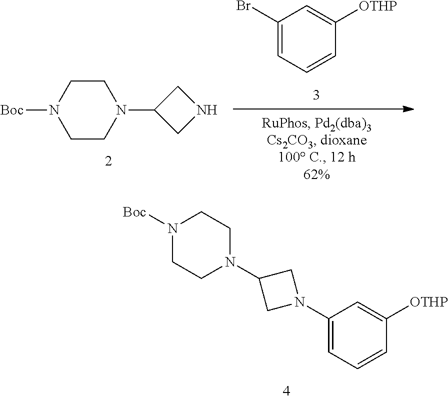

To a solution of tert-butyl 4-(azetidin-3-yl)piperazine-1-carboxylate (6.0 g, 25 mol, 1.0 equiv) in dioxane (60 mL) and 2-(3-bromophenoxy)tetrahydropyran (9.5 g, 37 μmol, 1.5 equiv) was added RuPhos (1.1 g, 2.4 mol, 0.1 equiv), Pd₂(dba)₃ (1.1 g, 1.2 mol, 0.1 equiv) and Cs₂CO₃ (24.3 g, 74.5 μmol, 3.0 equiv). The mixture was stirred at 100° C. for 12 h. The mixture was filtered and concentrated. The residue was purified by column chromatography (SiO₂, Petroleum ether/Ethyl acetate=10/1 to Dichloromethane: Methanol=10/1) to give tert-butyl 4-[1-(3-tetrahydropyran-2-yloxyphenyl)azetidin-3-yl]piperazine-1-carboxylate (6.5 g, 62% yield) as a yellow solid.

### 1.2 Synthesis of Compound 5

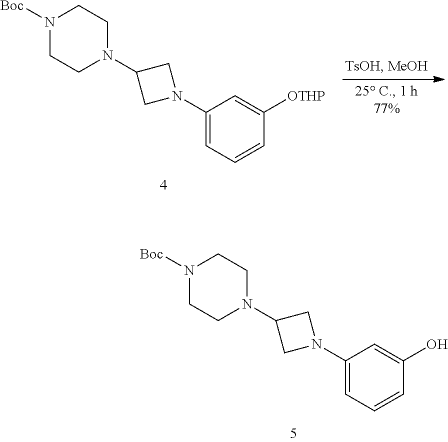

To a solution of tert-butyl 4-[1-(3-tetrahydropyran-2-yloxyphenyl)azetidin-3-yl]piperazine-1-carboxylate (6.5 g, 15.5 mol, 1.0 equiv) in MeOH (60 mL) was added TsOH (8.0 g, 46.7 μmol, 3.0 equiv). The mixture was stirred at 25° C. for 1 h. The mixture was quenched with 10 mL H₂O and extracted with EtOAc (3×10 mL), the combined organic layer was concentrated. The residue was purified by prep-TLC (SiO₂, Dichloromethane: Methanol=10:1) to give tert-butyl 4-[1-(3-hydroxyphenyl)azetidin-3-yl]piperazine-1-carboxylate (4.0 g, 77% yield) as a yellow solid.

### 1.3 Synthesis of Compound 7

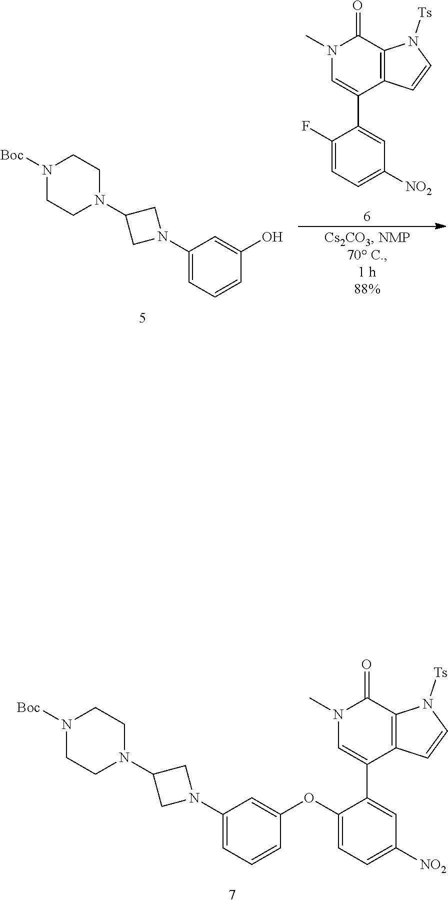

To a solution of tert-butyl 4-[1-(3-hydroxyphenyl)azetidin-3-yl]piperazine-1-carboxylate (500 mg, 1.5 μmol, 1.0 equiv) in NMP (1 mL) was added Cs₂CO₃ (977 mg, 3.0 μmol, 2.0 equiv) and 4-(2-fluoro-5-nitro-phenyl)-6-methyl-1-(p-tolylsulfonyl)pyrrolo[2,3-c]pyridin-7-one (661 mg, 1.5 μmol, 1.0 equiv). The mixture was stirred at 70° C. for 1 h and concentrated. The residue was used for next step without further purification.

### 1.4 Synthesis of Compound 8

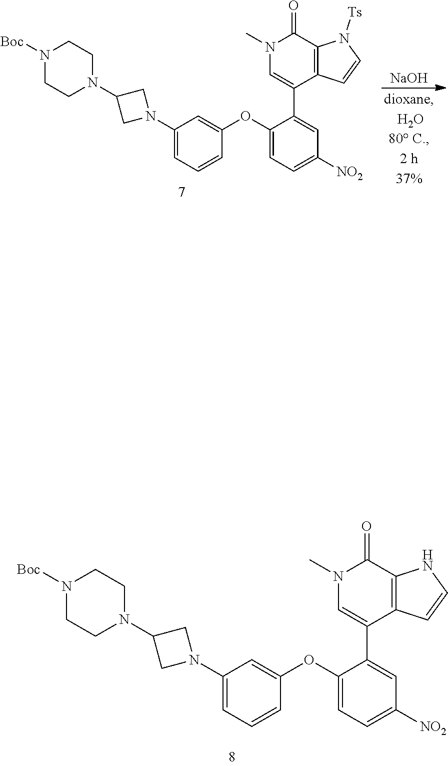

To a solution of tert-butyl 4-[1-[3-[2-[6-methyl-7-oxo-1-(p-tolylsulfonyl)pyrrolo [2,3-c]pyridin-4-yl]-4-nitro-phenoxy]phenyl]azetidin-3-yl]piperazine-1-carboxylate (1.0 g, 1.3 μmol, 1.0 equiv) in dioxane (5 mL) and H₂O (5 mL) was added NaOH (1.2 g, 30 μmol, 23.0 equiv). The mixture was stirred at 80° C. for 2 h. The residue was purified by prep-TLC (SiO₂, Dichloromethane: Methanol=10:1) to give tert-butyl 4-[1-[3-[2-(6-methyl-7-oxo-1H-pyrrolo[2,3-c]pyridin-4-yl)-4-nitro-phenoxy]phenyl]azetidin-3-yl]piperazine-1-carboxylate (300 mg, 37% yield).

### 1.5 Synthesis of Compound 9

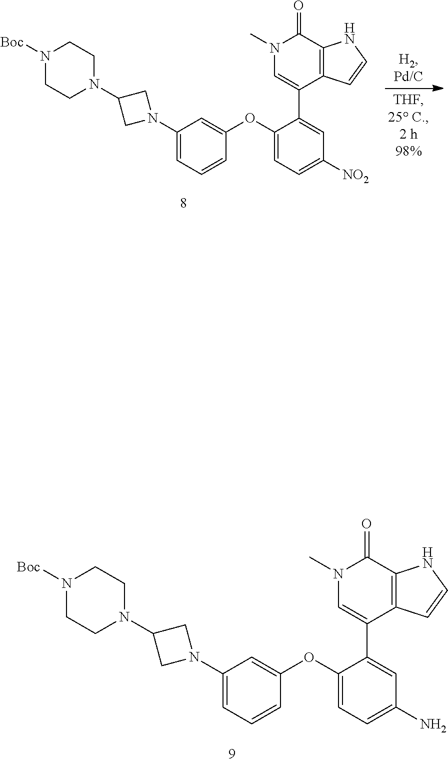 [SMILES: Cn1cc(-c2cc([N+](=O)[O-])ccc2Oc2cccc(N3CC(N4CCN(C(=O)OC(C)(C)C)CC4)C3)c2)c2cc[nH]c2c1=O ; Cn1cc(-c2cc(N)ccc2Oc2cccc(N3CC(N4CCN(C(=O)OC(C)(C)C)CC4)C3)c2)c2cc[nH]c2c1=O]

To a solution of tert-butyl 4-[1-[3-[2-(6-methyl-7-oxo-1H-pyrrolo[2,3-c]pyridin-4-yl)-4-nitro-phenoxy]phenyl]azetidin-3-yl]piperazine-1-carboxylate (320 mg, 532 mol, 1.0 equiv) in THF (5 mL) was added Pd/C (32 mg, 532 μmol, 10% purity, 1.0 equiv) under N₂ atmosphere. The mixture was stirred under H₂ at 25° C. for 2 h. The mixture was filtered and concentrated. The residue was used for next step directly without further purification.

### 1.6 Synthesis of Compound 10

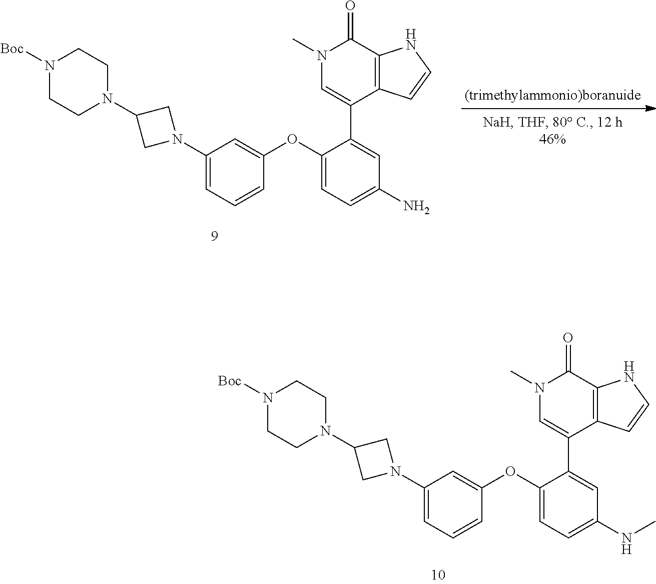 [SMILES: Cn1cc(-c2cc(N)ccc2Oc2cccc(N3CC(N4CCN(C(=O)OC(C)(C)C)CC4)C3)c2)c2cc[nH]c2c1=O ; CNc1ccc(Oc2cccc(N3CC(N4CCN(C(=O)OC(C)(C)C)CC4)C3)c2)c(-c2cn(C)c(=O)c3[nH]ccc23)c1]

To a solution of tert-butyl 4-[1-[3-[4-amino-2-(6-methyl-7-oxo-1H-pyrrolo[2,3-c]pyridin-4-yl)phenoxy]phenyl]azetidin-3-yl]piperazine-1-carboxylate (250 mg, 438 mol, 1.0 equiv) in THF (4 mL) was added (trimethylammonio)boranuide (95 mg, 1.3 μmol, 3.0 equiv) and NaH (70 mg, 1.7 mol, 60% purity, 4.0 equiv). The mixture was stirred at 80° C. for 12 h and poured into ice water (20 ml). The solution was extracted with EtOAc (3×10 mL), dried over Na₂SO₄, filtered and concentrated. The residue was purified by prep-TLC (SiO₂, Dichloromethane: Methanol=10:1) to give tert-butyl 4-[1-[3-[4-(methylamino)-2-(6-methyl-7-oxo-1H-pyrrolo[2,3-c]pyridin-4-yl)phenoxy]phenyl]azetidin-3-yl]piperazine-1-carboxylate (120 mg, 46% yield).

### 1.7 Synthesis of Compound 11

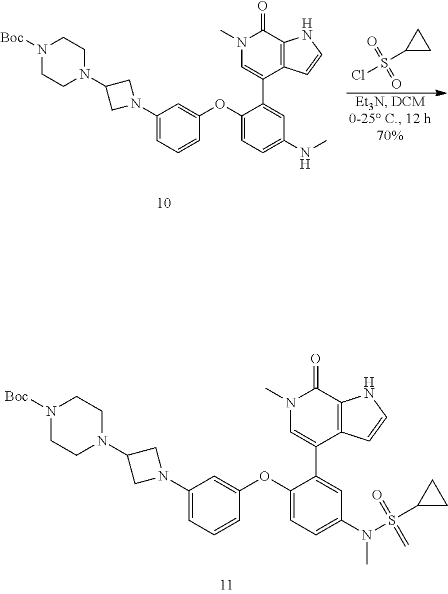 [SMILES: CNc1ccc(Oc2cccc(N3CC(N4CCN(C(=O)OC(C)(C)C)CC4)C3)c2)c(-c2cn(C)c(=O)c3[nH]ccc23)c1 ; O=S(=O)(Cl)C1CC1 ; C=S(=O)(C1CC1)N(C)c1ccc(Oc2cccc(N3CC(N4CCN(C(=O)OC(C)(C)C)CC4)C3)c2)c(-c2cn(C)c(=O)c3[nH]ccc23)c1]

To a solution of cyclopropanesulfonyl chloride (57 mg, 410 mol, 4.0 equiv) in DCM (1 mL) was added TEA (124 mg, 1.2 mol, 12.0 equiv) and tert-butyl 4-[1-[3-[4-(methylamino)-2-(6-methyl-7-oxo-1H-pyrrolo[2,3-c]pyridin-4-yl)phenoxy]phenyl]azetidin-3-yl]piperazine-1-carboxylate (60 mg, 102 μmol, 1.0 equiv) at 0° C. The mixture was stirred at 25° C. for 12 h and concentrated. The residue was purified by prep-TLC (SiO₂, EtOAc: MeOH=10:1). to give tert-butyl 4-[1-[3-[4-[cyclopropylsulfonyl(methyl)amino]-2-(6-methyl-7-oxo-1H-pyrrolo[2,3-c]pyridin-4-yl)phenoxy]phenyl]azetidin-3-yl]piperazine-1-carboxylate (50 mg, 70% yield).

### 1.8 Synthesis of Compound 12

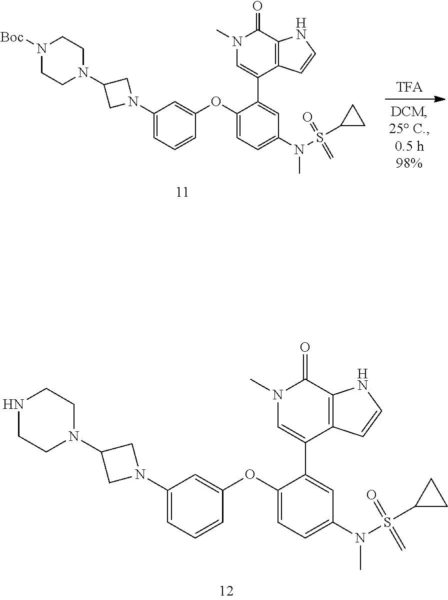 [SMILES: C=S(=O)(C1CC1)N(C)c1ccc(Oc2cccc(N3CC(N4CCN(C(=O)OC(C)(C)C)CC4)C3)c2)c(-c2cn(C)c(=O)c3[nH]ccc23)c1 ; C=S(=O)(C1CC1)N(C)c1ccc(Oc2cccc(N3CC(N4CCNCC4)C3)c2)c(-c2cn(C)c(=O)c3[nH]ccc23)c1 ; "TFA"]

To a solution of tert-butyl 4-[1-[3-[4-[cyclopropylsulfonyl(methyl)amino]-2-(6-methyl-7-oxo-1H-pyrrolo[2,3-c]pyridin-4-yl)phenoxy]phenyl]azetidin-3-yl]piperazine-1-carboxylate (50 mg, 72 μmol, 1.0 equiv) in DCM (3 mL) was added TFA (1 mL). The mixture was stirred at 25° C. for 0.5 h. The solution was filtered and concentrated. The residue was used directly for next step.

### 1.9 Synthesis of Compound I-88

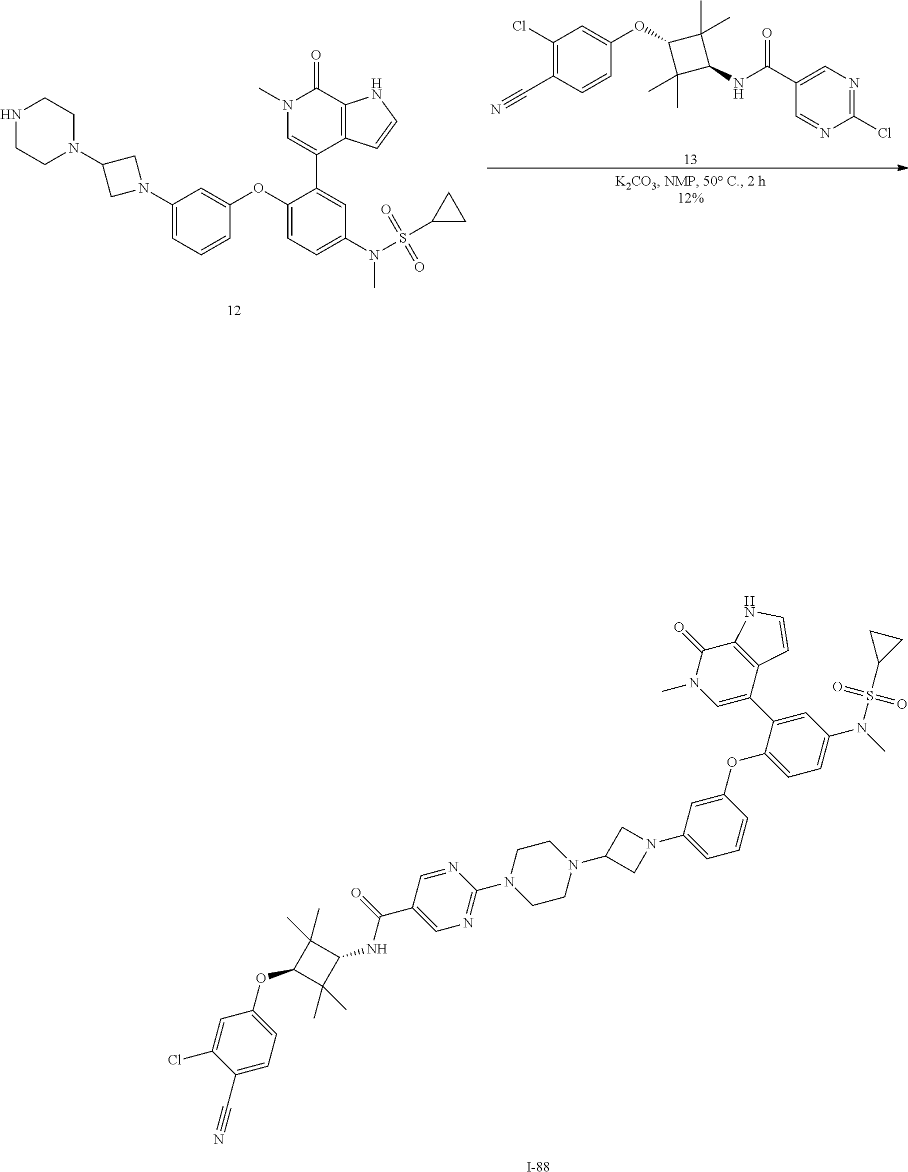

To a solution of N-methyl-N-[3-(6-methyl-7-oxo-1H-pyrrolo[2,3-c]pyridin-4-yl)-4-[3-(3-piperazin-1-ylazetidin-1-yl)phenoxy]phenyl]cyclopropanesulfonamide (50 mg, 71 mol, 1.0 equiv, TFA) and 2-chloro-N-[3-(3-chloro-4-cyano-phenoxy)-2,2,4,4-tetramethyl-cyclobutyl]pyrimidine-5-carboxamide (29 mg, 71 μmol, 1.0 equiv) in NMP (1 mL) was added K₂CO₃ (29 mg, 213 5 mol, 3.0 equiv). The mixture was stirred at 50 C for 2 h. The residue was purified by prep-HPLC (column: Waters Xbridge 150 mm*25 mm*5 μm; mobile phase: [water(NH₄HCO₃)-ACN]; B %: 600%-90%, 8 min) to give N-[3-(3-chloro-4-cyano-phenoxy)-2,2,4,4-tetramethyl-cyclobutyl]-2-[4-[1-[3-[4-[cyclopropylsulfonyl(methyl)amino]-2-(6-methyl-7-oxo-1H-pyrrolo[2,3-c]pyridin-4-yl)phenoxy]phenyl]azetidin-3-yl]piperazin-1l-yl]pyrimidine-5-carboxamide (9 mg, 12% o yield). ¹1HNMR (400 MHz, CDCl₃) δ 9.83 (s, 1H), 8.72 (s, 2H), 7.65-7.55 (m, 2H), 7.34-7.31 (m, 1H), 7.26-7.23 (m, 1H), 7.13 (s, 2H), 6.97 (s, 2H), 6.84-6.76 (m, 1H), 6.47 (t, J=2.4 Hz, 1H), 6.35-6.25 (m, 1H), 6.22-6.14 (m, 1H), 6.02 (d, J=2.1 Hz, 1H), 5.96 (d, J=8.0 Hz, 1H), 4.14 (d, J=8.0 Hz, 1H), 4.05 (s, 1H), 3.98-3.91 (m, 6H), 3.70-3.65 (m, 2H), 3.64 (s, 3H), 3.39 (s, 3H), 3.35-3.29 (m, 1H), 2.50-2.42 (m, 5H), 1.26 (s, 6H), 1.22 (s, 6H), 1.17-1.14 (m, 2H), 1.05-0.91 (m, 2H).

### Example 24—Synthesis of 2-(4-(1-(3-(4-amino-2-(6-methyl-7-oxo-6,7-dihydro-1H-pyrrolo[2,3-c]pyridin-4-yl)phenoxy)phenyl)azetidin-3-yl)piperazin-1-yl)-N-((1r, 3r)-3-(3-chloro-4-cyanophenoxy)-2,2,4,4-tetramethylcyclobutyl)pyrimidine-5-carboxamide (I-89)

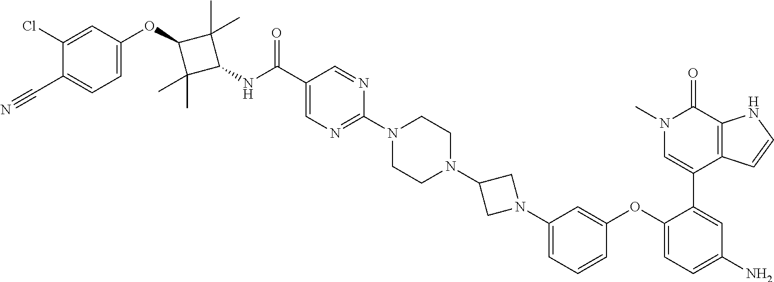 [SMILES: Cn1cc(-c2cc(N)ccc2Oc2cccc(N3CC(N4CCN(c5ncc(C(=O)N[C@H]6C(C)(C)[C@H](Oc7ccc(C#N)c(Cl)c7)C6(C)C)cn5)CC4)C3)c2)c2cc[nH]c2c1=O]

### 1.1 Synthesis of Compound 2

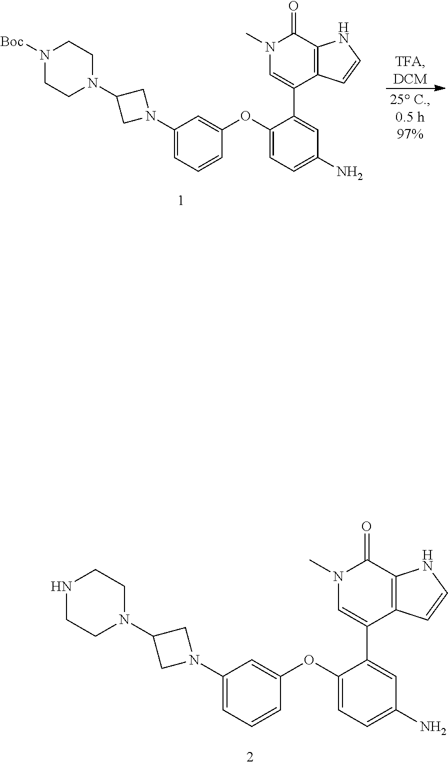

To a solution of tert-butyl 4-[1-[3-[4-amino-2-(6-methyl-7-oxo-1H-pyrrolo[2,3-c]pyridin-4-yl)phenoxy]phenyl]azetidin-3-yl]piperazine-1-carboxylate (50 mg, 87 μmol, 1.0 equiv) in DCM (3 mL) was added TFA (1 mL). The mixture was stirred at 25° C. for 0.5 h and concentrated. The residue was used directly for next step.

### 1.2 Synthesis of Compound I-89

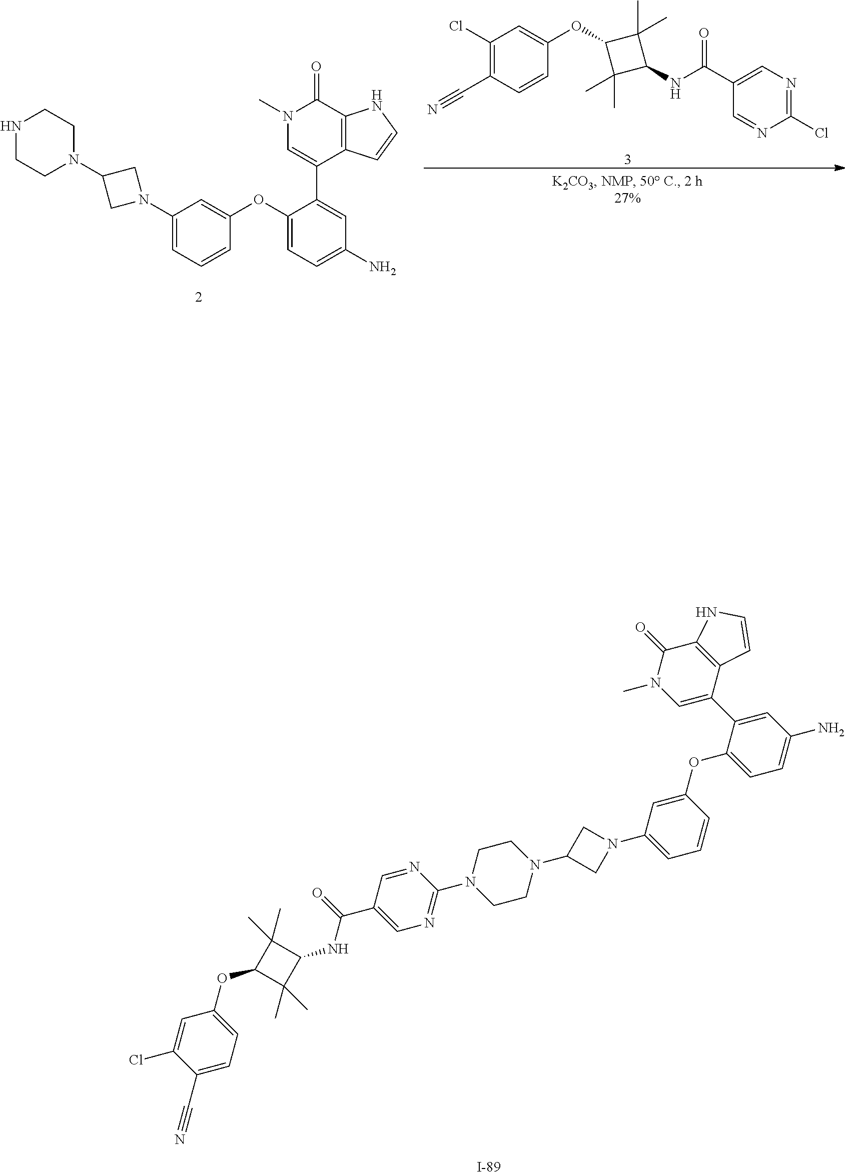

To a solution of 4-[5-amino-2-[3-(3-piperazin-1-ylazetidin-1-yl)phenoxy]phenyl]-6-methyl-1H-pyrrolo[2,3-c]pyridin-7-one (40 mg, 85 mol, 1.0 equiv) in NMP (1 mL) was added K₂CO₃ (35 mg, 255 μmol, 3.0 equiv) and 2-chloro-N-[3-(3-chloro-4-cyano-phenoxy)-2,2,4,4-tetramethyl-cyclobutyl]pyrimidine-5-carboxamide (35 mg, 85 μmol, 1.0 equiv). The mixture was stirred at 50° C. for 2 h and concentrated. The residue was purified by prep-HPLC (column: Phenomenex Synergi C18 150 mm*25 mm*10 μm; mobile phase: [water(FA)-ACN]; B %: 28%-58%, 10 min). to give 2-[4-[1-[3-[4-amino-2-(6-methyl-7-oxo-1H-pyrrolo [2,3-c]pyridin-4-yl)phenoxy]phenyl]azetidin-3-yl]piperazin-1-yl]-N-[3-(3-chloro-4-cyano-phenoxy)-2,2,4,4-tetramethyl-cyclobutyl]pyrimidine-5-carboxamide (20 mg, 27% yield). ¹HNMR (400 MHz, CDCl₃) δ 9.92-9.63 (m, 1H), 8.72 (s, 2H), 7.58 (d, J=8.8 Hz, 1H), 7.21 (t, J=2.8 Hz, 1H), 7.06-6.92 (m, 4H), 6.88 (d, J=2.8 Hz, 1H), 6.84-6.76 (m, 1H), 6.75-6.62 (m, 1H), 6.44 (t, J=2.4 Hz, 1H), 6.23-6.14 (m, 1H), 6.10-6.00 (m, 1H), 5.99-5.91 (m, 1H), 5.88 (s, 1H), 4.14 (d, J=8.0 Hz, 1H), 4.05 (s, 1H), 3.98-3.91 (m, 4H), 3.90-3.83 (m, 2H), 3.65-3.59 (m, 2H), 3.58 (s, 3H), 3.33-3.23 (m, 1H), 2.52-2.33 (m, 4H), 1.24 (d, J=13.2 Hz, 12H).

### Example 25—Synthesis of N-((1r, 3r)-3-(3-chloro-4-cyanophenoxy)-2,2,4,4-tetramethylcyclobutyl)-2-(4-(1-(3-(2-(6-methyl-7-oxo-6,7-dihydro-1H-pyrrolo[2,3-c]pyridin-4-yl)phenoxy)phenyl)azetidin-3-yl)piperazin-1-yl)pyrimidine-5-carboxamide (I-90)

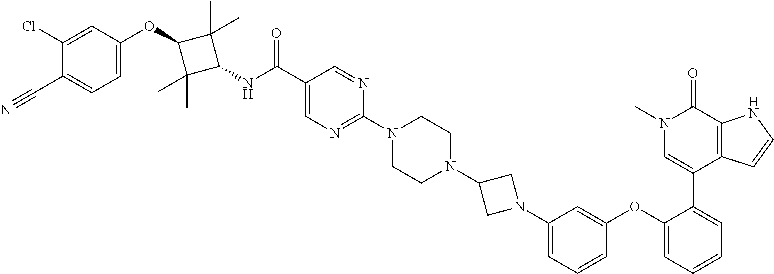 [SMILES: Cn1cc(-c2ccccc2Oc2cccc(N3CC(N4CCN(c5ncc(C(=O)N[C@H]6C(C)(C)[C@H](Oc7ccc(C#N)c(Cl)c7)C6(C)C)cn5)CC4)C3)c2)c2cc[nH]c2c1=O]

### 1.1 Synthesis of Compound 3

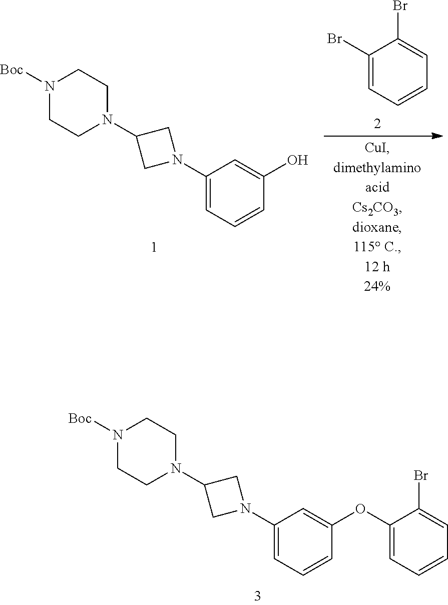 [SMILES: CC(C)(C)OC(=O)N1CCN(C2CN(c3cccc(O)c3)C2)CC1 ; Brc1ccccc1Br ; CC(C)(C)OC(=O)N1CCN(C2CN(c3cccc(Oc4ccccc4Br)c3)C2)CC1]

A mixture of CuI (57 mg, 299 μmol, 0.2 equiv), Cs₂CO₃ (977 mg, 3.00 μmol, 2 equiv), 1,2-dibromobenzene (389 mg, 1.65 μmol, 196 μL, 1.1 equiv), tert-butyl 4-[1-(3-hydroxyphenyl)azetidin-3-yl]piperazine-1-carboxylate (500 mg, 1.50 μmol, 1 equiv) and 2-(dimethylamino)acetic acid (92 mg, 899 μmol, 0.6 equiv) in dioxane (20 mL) was stirred at 25° C. for 30 min under N₂, then the mixture was stirred at 115° C. for 12 h. The reaction mixture was filtered and concentrated under reduced pressure to give a residue. The residue was purified by prep-HPLC (column: Waters Xbridge C18 150 mm*50 mm*10 μm; mobile phase: [water (NH₄HCO₃)-ACN]; B %: 57%-87%, 10 min) to give the compound tert-butyl 4-[1-[3-(2-bromophenoxy)phenyl]azetidin-3-yl]piperazine-1-carboxylate (180 mg, 368 μmol, 24% yield) as a yellow solid.

### 1.2 Synthesis of Compound 5

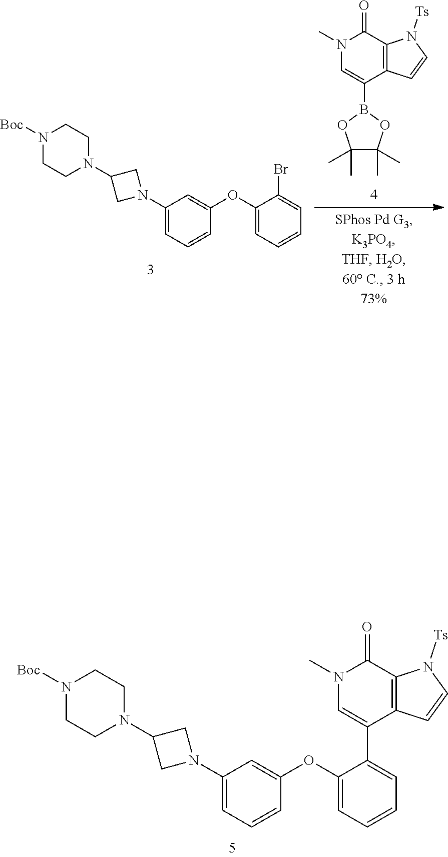 [SMILES: CC(C)(C)OC(=O)N1CCN(C2CN(c3cccc(Oc4ccccc4Br)c3)C2)CC1 ; Cc1ccc(S(=O)(=O)n2ccc3c(B4OC(C)(C)C(C)(C)O4)cn(C)c(=O)c32)cc1 ; Cc1ccc(S(=O)(=O)n2ccc3c(-c4ccccc4Oc4cccc(N5CC(N6CCN(C(=O)OC(C)(C)C)CC6)C5)c4)cn(C)c(=O)c32)cc1]

To a solution of tert-butyl 4-[1-[3-(2-bromophenoxy)phenyl]azetidin-3-yl]piperazine-1-carboxylate (140 mg, 286.64 mol, 1 eq) and 6-methyl-1-(p-tolylsulfonyl)-4-(4,4,5,5-tetramethyl-1,3,2-dioxaborolan-2-yl)pyrrolo[2,3-c]pyridin-7-one (135 mg, 315 mol, 1.1 equiv) in H₂O (1 mL) and THF (5 mL) was added SPhos Pd G3 (22 mg, 28 μmol, 0.1 equiv) and K₃PO₄ (182 mg, 859 mol, 3 equiv). The mixture was stirred at 60° C. for 3 h. The reaction mixture was filtered and concentrated under reduced pressure to give a residue. The residue was purified by prep-TLC (SiO₂, PE: EA=1:1) to give the compound tert-butyl 4-[1-[3-[2-[6-methyl-7-oxo-1-(p-tolylsulfonyl)pyrrolo[2,3-c]pyridin-4-yl]phenoxy]phenyl]azetidin-3-yl]piperazine-1-carboxylate (150 mg, 211 gmol, 73% yield) as a yellow solid.

### 1.3 Synthesis of Compound 6

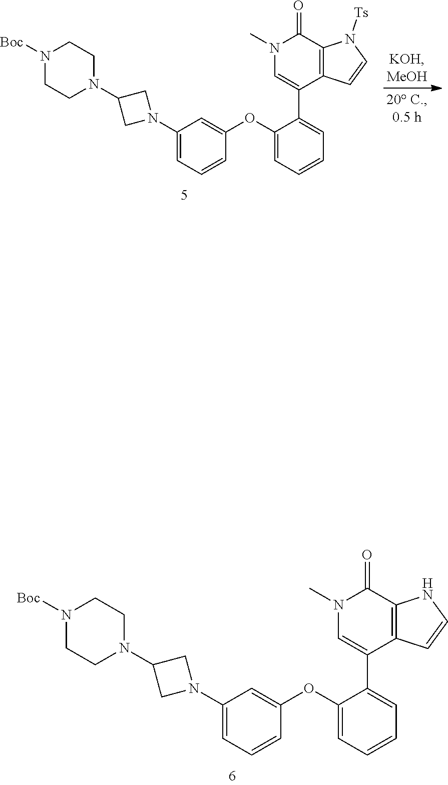 [SMILES: Cc1ccc(S(=O)(=O)n2ccc3c(-c4ccccc4Oc4cccc(N5CC(N6CCN(C(=O)OC(C)(C)C)CC6)C5)c4)cn(C)c(=O)c32)cc1 ; Cn1cc(-c2ccccc2Oc2cccc(N3CC(N4CCN(C(=O)OC(C)(C)C)CC4)C3)c2)c2cc[nH]c2c1=O]

To a solution of tert-butyl 4-[1-[3-[2-[6-methyl-7-oxo-1-(p-tolylsulfonyl) pyrrolo[2,3-c]pyridin-4-yl]phenoxy]phenyl]azetidin-3-yl]piperazine-1-carboxylate (70 mg, 98 μmol, 1 equiv) in MeOH (2 mL) was added KOH (16 mg, 295 mol, 3 equiv). The mixture was stirred at 20° C. for 0.5 h. The reaction mixture was quenched by addition water 30 mL, and then extracted with EA 50 mL. The combined organic layers were washed with brine 100 mL, dried over anhydrous sodium sulfate, filtered and concentrated under reduced pressure to give the compound tert-butyl 4-[1-[3-[2-(6-methyl-7-oxo-1H-pyrrolo[2,3-c]pyridin-4-yl) phenoxy]phenyl]azetidin-3-yl]piperazine-1-carboxylate (50 mg, crude) as a yellow oil.

### 1.4 Synthesis of Compound 7

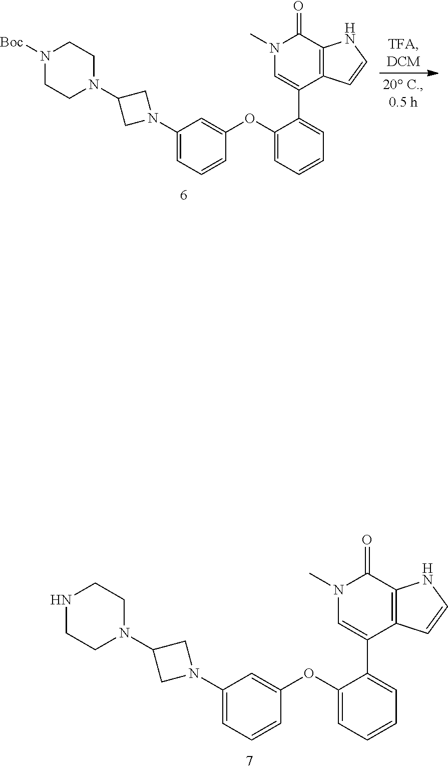 [SMILES: Cn1cc(-c2ccccc2Oc2cccc(N3CC(N4CCN(C(=O)OC(C)(C)C)CC4)C3)c2)c2cc[nH]c2c1=O ; Cn1cc(-c2ccccc2Oc2cccc(N3CC(N4CCNCC4)C3)c2)c2cc[nH]c2c1=O]

To a solution of tert-butyl 4-[1-[3-[2-(6-methyl-7-oxo-1H-pyrrolo[2,3-c]pyridin-4-yl)phenoxy]phenyl]azetidin-3-yl]piperazine-1-carboxylate (50 mg, 89 μmol, 1 equiv) in DCM (2 mL) was added TFA (1.54 g, 1.00 mL). The mixture was stirred at 20° C. for 0.5 h. The reaction mixture was filtered and concentrated under reduced pressure to give the compound 6-methyl-4-[2-[3-(3-piperazin-1-ylazetidin-1-yl)phenoxy]phenyl]-1H-pyrrolo[2,3-c]pyridin-7-one (50 mg, crude, TFA) as a yellow oil.

### 1.5 Synthesis of Compound I-90

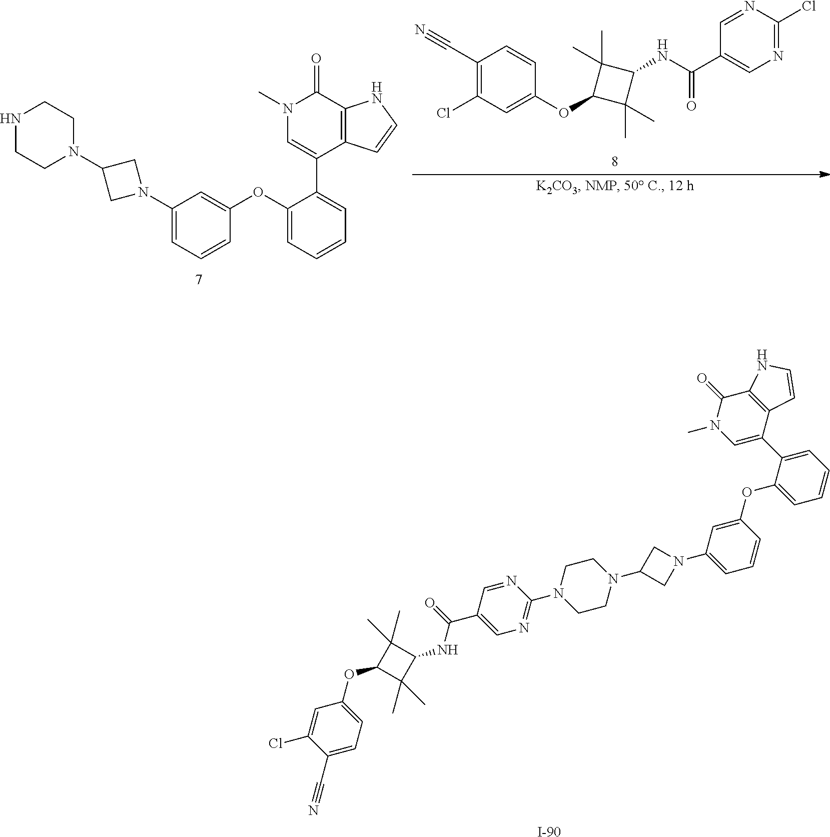

To a solution of 6-methyl-4-[2-[3-(3-piperazin-1-ylazetidin-1-yl)phenoxy]phenyl]-1H-pyrrolo[2,3-c]pyridin-7-one (50 mg, 87 gmol, 1 equiv, TFA) in NMP (2 mL) was added K₂CO₃ (36 mg, 263 mol, 3 equiv) and 2-chloro-N-[3-(3-chloro-4-cyano-phenoxy)-2,2,4,4-tetramethyl-cyclobutyl]pyrimidine-5-carboxamide (36 mg, 87 μmol, 1 equiv). The mixture was stirred at 50° C. for 12 h. The reaction mixture was filtered and concentrated under reduced pressure to give a residue. The residue was purified by prep-HPLC (column: Waters Xbridge 150 mm*25 mm*5 μm; mobile phase: [water(NH₄HCO₃)-ACN]; B %: 63%-93%, 8 min) to give the compound N-[3-(3-chloro-4-cyano-phenoxy)-2,2,4,4-tetramethyl-cyclobutyl]-2-[4-[1-[3-[2-(6-methyl-7-oxo-1H-pyrrolo[2,3-c]pyridin-4-yl)phenoxy]phenyl]azetidin-3-yl]piperazin-1-yl]pyrimidine-5-carboxamide (29.0 mg, 39% yield) as an off-white solid. ¹H NMR (400 MHz, DMSO-d₆): δ 12.01 (s, 1H), 8.78 (s, 2H), 7.91 (d, 1H, J=8.8 Hz), 7.74 (d, 1H, J=9.2 Hz), 7.50-7.48 (m, 1H), 7.39-7.35 (m, 1H), 7.27-7.22 (m, 4H), 7.09-7.00 (m, 3H), 6.22-6.21 (m, 2H), 6.12 (d, 1H, J=6.4 Hz), 5.92 (s, 1H), 4.30 (s, 1H), 4.05 (d, 1H, J=9.2 Hz), 3.85-3.84 (m, 6H), 3.54-3.52 (m, 5H), 3.26-3.22 (m, 2H), 2.39 (s, 3H), 1.22 (s, 6H), 1.12 (s, H).

### Example 26—Synthesis of N-((1r, 3r)-3-(3-chloro-4-cyanophenoxy)-2,2,4,4-tetramethylcyclobutyl)-2-(4-(1-(2-fluoro-3-(4-(2-hydroxypropan-2-yl)-2-(6-methyl-7-oxo-6,7-dihydro-1H-pyrrolo[2,3-c]pyridin-4-yl)phenoxy)phenyl)azetidin-3-yl)piperazin-1-yl)pyrimidine-5-carboxamide (I-92)

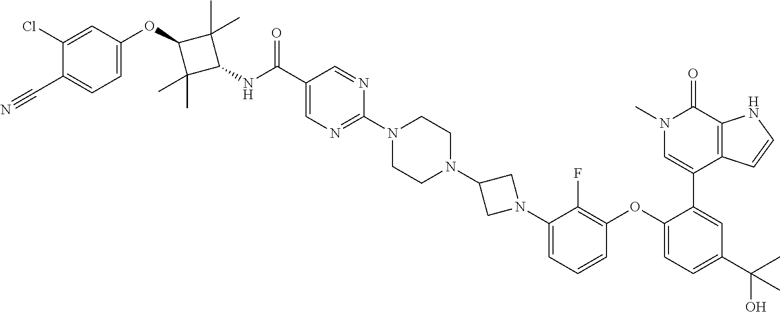 [SMILES: Cn1cc(-c2cc(C(C)(C)O)ccc2Oc2cccc(N3CC(N4CCN(c5ncc(C(=O)N[C@H]6C(C)(C)[C@H](Oc7ccc(C#N)c(Cl)c7)C6(C)C)cn5)CC4)C3)c2F)c2cc[nH]c2c1=O]

### 1.1 Synthesis of Compound 2

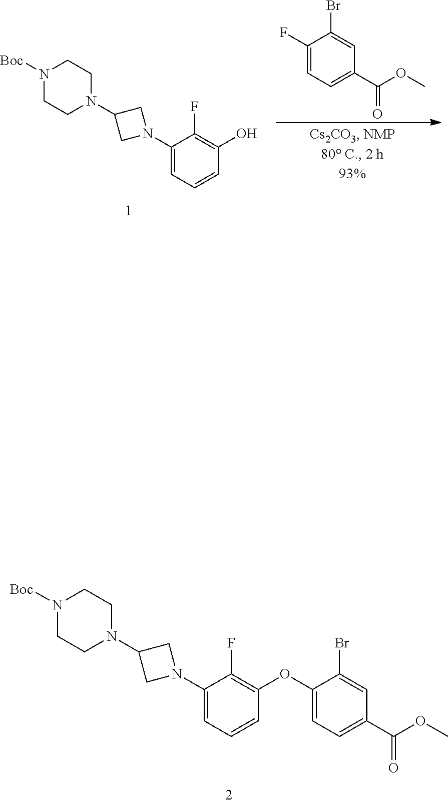 [SMILES: CC(C)(C)OC(=O)N1CCN(C2CN(c3cccc(O)c3F)C2)CC1 ; COC(=O)c1ccc(F)c(Br)c1 ; COC(=O)c1ccc(Oc2cccc(N3CC(N4CCN(C(=O)OC(C)(C)C)CC4)C3)c2F)c(Br)c1]

A mixture of tert-butyl 4-[1-(2-fluoro-3-hydroxy-phenyl)azetidin-3-yl]piperazine-1-carboxylate (400 mg, 1.14 μmol, 1.0 equiv) and methyl 3-bromo-4-fluoro-benzoate (292 mg, 1.25 μmol, 1.1 equiv) and Cs₂CO₃ (742 mg, 2.3 μmol, 2.0 equiv) in NMP (1.5 mL) was stirred at 80° C. for 2 h. The mixture was purified by prep-TLC (SiO₂, Petroleum ether: Ethyl acetate=1:1) to give tert-butyl 4-[1-[3-(2-bromo-4-methoxycarbonyl-phenoxy)-2-fluoro-phenyl]azetidin-3-yl]piperazine-1-carboxylate (600 mg, 93% yield). ¹H NMR (400 MHz, CDCl₃): δ 8.59 (d, J=2.0 Hz, 1H), 7.91-7.86 (m, 1H), 7.03-6.95 (m, 1H), 6.79-6.74 (m, 1H), 6.55-6.48 (m, 1H), 6.41-6.34 (m, 1H), 4.15-4.07 (m, 2H), 3.92 (s, 3H), 3.88-3.81 (m, 2H), 3.54-3.45 (m, 4H), 3.36-3.26 (m, 1H), 2.41-2.32 (m, 4H), 1.49 (s, 9H).

### 1.2 Synthesis of Compound 3

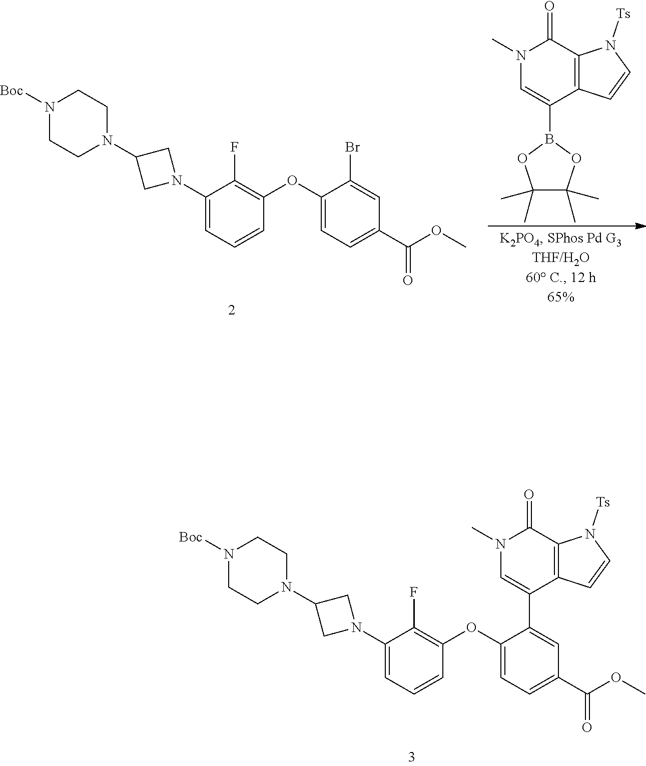 [SMILES: COC(=O)c1ccc(Oc2cccc(N3CC(N4CCN(C(=O)OC(C)(C)C)CC4)C3)c2F)c(Br)c1 ; Cc1ccc(S(=O)(=O)n2ccc3c(B4OC(C)(C)C(C)(C)O4)cn(C)c(=O)c32)cc1 ; COC(=O)c1ccc(Oc2cccc(N3CC(N4CCN(C(=O)OC(C)(C)C)CC4)C3)c2F)c(-c2cn(C)c(=O)c3c2ccn3S(=O)(=O)c2ccc(C)cc2)c1]

To a solution of tert-butyl 4-[1-[3-(2-bromo-4-methoxycarbonyl-phenoxy)-2-fluoro-phenyl]azetidin-3-yl]piperazine-1-carboxylate (600 mg, 1.1 mol, 1.0 equiv) and 6-methyl-1-(p-tolylsulfonyl)-4-(4,4,5,5-tetramethyl-1,3,2-dioxaborolan-2-yl)pyrrolo[2,3-c]pyridin-7-one (500 mg, 1.17 μmol, 1.1 equiv) in THF (30 mL) and H₂O (δ mL) was added K₃PO₄ (678 mg, 3.2 mol, 3.0 equiv) and SPhos Pd G3 (84 mg, 108 mol, 1.01e-¹ eq). The mixture was stirred at 60° C. for 12 h under N₂. The mixture was concentrated and purified by prep-TLC (SiO₂, Petroleum ether: Ethyl acetate=0:1) to give tert-butyl 4-[1-[2-fluoro-3-[4-methoxy carbonyl-2-[6-methyl-7-oxo-1-(p-tolylsulfonyl)pyrrolo[2,3-c]pyridin-4-yl]phenoxy]phenyl]azetidin-3-yl]piperazine-1-carboxylate (540 mg, 65% yield).

### 1.3 Synthesis of Compound 4

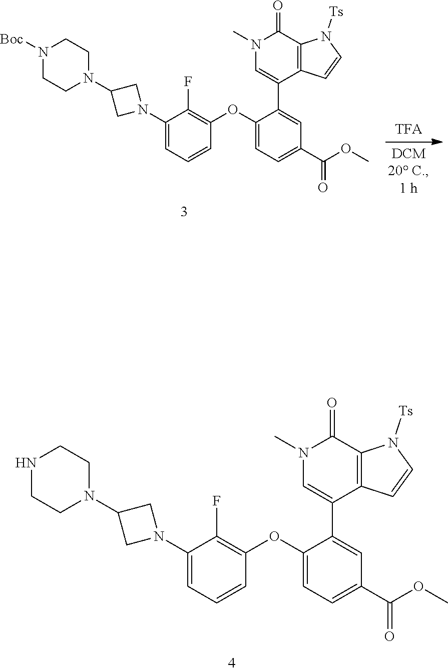 [SMILES: COC(=O)c1ccc(Oc2cccc(N3CC(N4CCN(C(=O)OC(C)(C)C)CC4)C3)c2F)c(-c2cn(C)c(=O)c3c2ccn3S(=O)(=O)c2ccc(C)cc2)c1 ; COC(=O)c1ccc(Oc2cccc(N3CC(N4CCNCC4)C3)c2F)c(-c2cn(C)c(=O)c3c2ccn3S(=O)(=O)c2ccc(C)cc2)c1]

The solution of tert-butyl 4-[1-[2-fluoro-3-[4-methoxycarbonyl-2-[6-methyl-7-oxo-1-(p-tolylsulfonyl)pyrrolo[2,3-c]pyridin-4-yl]phenoxy]phenyl]azetidin-3-yl]piperazine-1-carboxylate (540 mg, 687.13 μmol, 1 equiv) in TFA (1 mL) and DCM (3 mL) was stirred at 20° C. for 1 h. The solution was concentrated under reduced pressure to give methyl 4-[2-fluoro-3-(3-piperazin-1-ylazetidin-1-yl)phenoxy]-3-[6-methyl-7-oxo-1-(p-tolylsulfonyl) pyrrolo[2,3-c]pyridin-4-yl]benzoate (550 mg, crude, TFA salt).

### 1.4 Synthesis of Compound 5

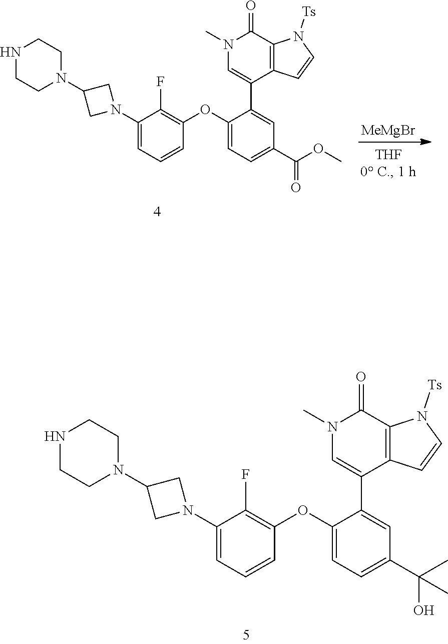 [SMILES: "MeMgBr" ; COC(=O)c1ccc(Oc2cccc(N3CC(N4CCNCC4)C3)c2F)c(-c2cn(C)c(=O)c3c2ccn3S(=O)(=O)c2ccc(C)cc2)c1 ; Cc1ccc(S(=O)(=O)n2ccc3c(-c4cc(C(C)(C)O)ccc4Oc4cccc(N5CC(N6CCNCC6)C5)c4F)cn(C)c(=O)c32)cc1]

To a solution of methyl 4-[2-fluoro-3-(3-piperazin-1-ylazetidin-1-yl)phenoxy]-3-[6-methyl-7-oxo-1-(p-tolylsulfonyl)pyrrolo[2,3-c]pyridin-4-yl]benzoate (100 mg, 146 mol, 1.0 equiv) in THF (1 mL) was added MeMgBr (3 M, 0.3 mL, 6.2 eq) at 0° C. The solution was stirred at 0° C. for 1 h and used directly for next step.

### 1.5 Synthesis of Compound 6

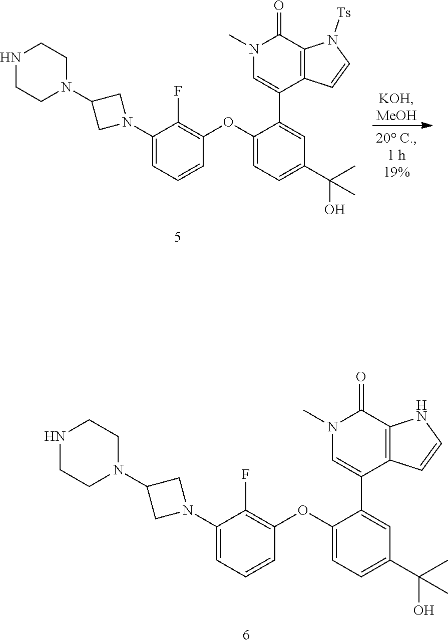

The mixture of 4-[2-[2-fluoro-3-(3-piperazin-1-ylazetidin-1-yl)phenoxy]-5-(1-hydroxy-1-methyl-ethyl)phenyl]-6-methyl-1-(p-tolylsulfonyl)pyrrolo[2,3-c]pyridin-7-one (100 mg, 146 mol, 1.0 equiv) and KOH (164 mg, 2.92 μmol, 20 equiv) in MeOH (1 mL) was stirred at 20° C. for 1 h. The mixture was filtered and concentrated. The residue was purified by prep-HPLC(column: Phenomenex Synergi C18 150 mm*25 mm*10 μm; mobile phase: [water(FA)-ACN]; B %: 9%-39%, 10 min) to give 4-[2-[2-fluoro-3-(3-piperazin-1-ylazetidin-1-yl)phenoxy]-5-(1-hydroxy-1-methyl-ethyl)phenyl]-6-methyl-¹H-pyrrolo[2,3-c]pyridin-7-one (15 mg. 19% yield). ¹H NMR (400 MHz, MeOD): δ 8.63-8.48 (m, 1H), 7.68-7.62 (m, 1H), 7.48-7.44 (m, 1H), 7.34-7.27 (m, 1H), 7.22 (s, 1H), 6.97-6.90 (m, 1H), 6.81-6.74 (m, 1H), 6.38-6.32 (m, 1H), 6.29-6.16 (m, 2H), 4.08-4.00 (m, 2H), 3.77-3.68 (m, 3H), 3.67-3.61 (m, 3H), 3.13-3.07 (m, 4H), 2.59-2.49 (m, 4H), 1.58 (s, 6H).

### 1.6 Synthesis of I-92

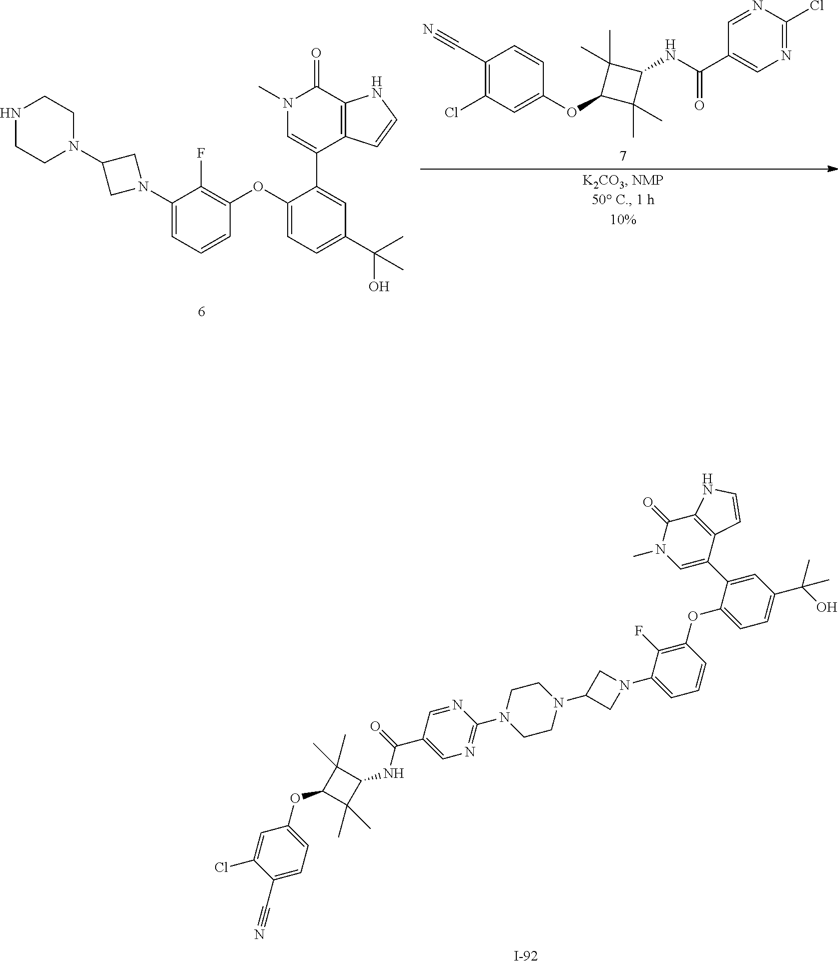

The mixture of N-[4-[2-fluoro-3-(3-piperazin-1-ylazetidin-1-yl)phenoxy]-3-(6-methyl-7-oxo-1H-pyrrolo[2,3-c]pyridin-4-yl)phenyl]-N-methyl-cyclopropanesulfonamide (51 mg, 71 μmol, 1.0 equiv, TFA salt) and 2-chloro-N-[3-(3-chloro-4-cyano-phenoxy)-2,2,4,4-tetramethyl-cyclobutyl]pyrimidine-5-carboxamide (30 mg, 72 μmol, 1.0 equiv) and K₂CO₃(49 mg, 353 mol, 5.0 equiv) in NMP (1.5 mL) was stirred at 50° C. for 1 h. The mixture was purified by prep-HPLC (column: Phenomenex Synergi C18 150 mm*25 mm*10 μm; mobile phase: [water(FA)-ACN]; B %: 44%-64%, 1 0 min) to give N-[3-(3-chloro-4-cyano-phenoxy)-2,2,4,4-tetramethyl-cyclobutyl]-2-[4-[1-[3-[4-[cyclopropylsulfonyl(methyl)amino]-2-(6-methyl-7-oxo-1H-pyrrolo[2,3-c]pyridin-4-yl)phenoxy]-2-fluoro-phenyl]azetidin-3-yl]piperazin-1-yl]pyrimidine-5-carboxamide (9.86 mg, 13% yield). ¹H NMR (400 MHz, CDCl₃): δ 9.73-9.57 (m, 1H), 8.72 (s, 2H), 7.66-7.62 (m, 1H), 7.58 (d, J=8.8 Hz, 1H), 7.43-7.38 (m, 1H), 7.24-7.21 (m, 1H), 7.18 (s, 1H), 6.98-6.95 (m, 1H), 6.92-6.76 (m, 3H), 6.46-6.42 (m, 1H), 6.37-6.30 (m, 1H), 6.26-6.18 (m, 1H), 5.98-5.90 (m, 1H), 4.18-3.80 (m, 9H), 3.74-3.63 (m, 3H), 3.36-3.26 (m, 1H), 2.54-2.41 (m, 4H), 1.64 (s, 6H), 1.26 (s, 6H), 1.22 (s, 6H).

### Example 27—LC-MS Physical Characterization Data

Exemplary compounds were analyzed by LC-MS. Results are provided below.

TABLE 2

| Compound | Observed | Retention | LCMS |
|---|---|---|---|
| No. | Mass (m/z) | Time (min) | Method |
| I-1 | 440.1, 879.2 | 2.961 | 25 |
| I-2 | 484.2, 967.3 | 2.939 | 25 |
| I-3 | 528.3, 1055.3 | 2.906 | 25 |
| I-4 | 572.2, 1143.3 | 2.893 | 25 |
| I-5 | 510.2, 1019.3 | 2.257 | 25 |
| I-6 | 475.0, 949.3 | 3.056 | 25 |
| I-7 | 519.1, 1037.3 | 2.997 | 25 |
| I-8 | 563.2, 1125.3 | 2.964 | 25 |
| I-9 | 607.8, 1213.3 | 2.955 | 25 |
| I-10 | 545.2, 1089.3 | 2.349 | 25 |
| I-11 | 949.2 | 2.954 | 25 |
| I-12 | 511.1, 1021.3 | 3.188 | 25 |
| I-13 | 497.2, 993.3 | 3.057 | 25 |
| I-14 | 1007.2, 504.1 | 3.092 | 25 |
| I-15 | 489.3 | 3.234 | 25 |
| I-16 | 504.1 | 3.107 | 25 |
| I-17 | 963.2 | 3.163 | 25 |
| I-18 | 919.3 | 3.097 | 25 |
| I-19 | 467.3, 933.2 | 3.113 | 25 |
| I-20 | 474.1, 947.3 | 3.184 | 25 |
| I-21 | 481.2, 961.3 | 2.901 | 40 |
| I-22 | 975.3 | 3.312 | 25 |
| I-23 | 452.1, 903.3 | 3.204 | 25 |
| I-24 | 453.1 | 3.106 | 25 |
| I-25 | 919.2 | 2.788 | 40 |
| I-26 | 933.2 | 2.867 | 40 |
| I-27 | 474.2, 947.3 | 3.017 | 40 |
| I-28 | 481.2, 961.3 | 3.062 | 40 |
| I-29 | 488.1 | 3.152 | 40 |
| I-30 | 877.2 | 2.918 | 25 |
| I-31 | 890.2 | 2.906 | 25 |
| I-32 | 516.3, 1031.3 | 2.349 | 25 |
| I-33 | 523.2, 1045.3 | 1.713 | 40 |
| I-34 | 530.2 | 1.703 | 40 |
| I-35 | 500.3, 999.3 | 2.455 | 25 |
| I-36 | 493.6, 986.3 | 2.278 | 40 |
| I-37 | 987.3 | 1.825 | 40 |
| I-38 | 487.6, 974.3 | 1.834 | 40 |
| I-39 | 494.2, 987.3 | 2.45 | 25 |
| I-40 | 486.7 | 3.112 | 40 |
| I-41 | 494.1, 987.5 | 2.439 | 25 |
| I-42 | 494.3, 987.3 | 2.424 | 25 |
| I-43 | 974.3 | 2.398 | 25 |
| I-44 | 945.3 | 3.281 | 25 |
| I-45 | 487.1 | 3.054 | 40 |
| I-46 | 950.2 | 2.999 | 25 |
| I-47 | 475.6, 950.2 | 3.054 | 25 |
| I-48 | 961.3 | 3.102 | 25 |
| I-49 | 488.1, 975.3 | 3.225 | 25 |
| I-50 | 468.2, 935.3 | 2.71 | 40 |
| I-51 | 949.2 | 2.764 | 40 |
| I-52 | 482.1, 963.3 | 2.861 | 40 |
| I-53 | 934.2 | 3.05 | 25 |
| I-54 | 474.6, 948.2 | 3.09 | 25 |
| I-55 | 467.8, 934.2 | 3.169 | 25 |
| I-56 | 450.6, 900.1 | 3.158 | 25 |
| I-57 | 450.1, 899.3 | 3.038 | 25 |
| I-58 | 457.3, 913.3 | 2.825 | 40 |
| I-59 | 913.3 | 3.096 | 25 |
| I-60 | 464.1, 927.3 | 2.888 | 40 |
| I-61 | 421.7, 842.3 | 2.951 | 40 |
| I-62 | 429.2, 857.2 | 2.599 | 25 |
| I-63 | 887.3 | 2.978 | 40 |
| I-64 | 484.2, 967.2 | 3.088 | 25 |
| I-65 | 493.2, 985.3 | 3.116 | 25 |
| I-66 | 495.7, 990.2 | 2.867 | 25 |
| I-67 | 471.2, 941.3 | 3.036 | 25 |
| I-68 | 471.2, 941.3 | 2.995 | 25 |
| I-69 | 474.3, 947.3 | 2.582 | 40 |
| I-70 | 474.6, 948.3 | 2.607 | 25 |
| I-71 | 949.3 | 3.139 | 25 |
| I-72 | 474.8, 948.3 | 2.738 | 40 |
| I-73 | 475.2, 949.3 | 3.017 | 25 |
| I-74 | 472.7, 944.3 | 3.18 | 25 |
| I-75 | 945.2 | 2.37 | 25 |
| I-76 | 472.5, 944.2 | 2.365 | 25 |
| I-77 | 934.2 | 2.352 | 25 |
| I-78 | 958.2 | 2.414 | 25 |
| I-79 | 479.7, 958.3 | 2.879 | 40 |
| I-80 | 480.7, 960.3 | 3.106 | 25 |
| I-81 | 488.4, 975.3 | 3.137 | 25 |
| I-82 | 494.9, 988.3 | 2.707 | 25 |
| I-83 | 481.3, 961.4 | 2.396 | 25 |
| I-84 | 474.8, 948.3 | 2.892 | 25 |
| I-85 | 474.6, 948.3 | 2.36 | 25 |
| I-86 | 488.2, 975.3 | 2.422 | 25 |
| I-87 | 481.8, 962.3 | 2.379 | 25 |
| I-88 | 486.1, 971.3 | 2.496 | 25 |
| I-89 | 853.3 | 2.042 | 25 |
| I-90 | 838.3 | 1.925 | 40 |
| I-91 | 495.4, 989.3 | 2.522 | 25 |
| I-92 | 458.2, 914.3 | 1.871 | 40 |
| I-93 | 871.3 | 2.073 | 25 |
| I-94 | 428.7, 856.3 | 2.534 | 25 |
| I-95 | 480.8, 960.3 | 1.888 | 40 |
| I-96 | 885.3 | 1.747 | 40 |
| I-97 | 421.6, 842.3 | 2.033 | 25 |
| I-98 | 414.2, 827.3 | 1.897 | 40 |
| I-99 | 452.3, 903.4 | 1.771 | 40 |
| I-100 | 430.7, 860.3 | 2.044 | 25 |
| I-101 | 841.6 | 3.015 | 05 |
| I-102 | 826.5 | 3.499 | 05 |
| I-103 | 859.6 | 2.995 | 05 |

### Example 28—Synthesis of Additional Compounds

The following additional compounds were prepared based on procedures described herein: compounds 1-2, 1-3, 1-4, 1-5, 1-6, 1-7, 1-8, 1-9, 1-10, I-11, 1-12, 1-13, 1-14, I-15, 1-16, I-17, 1-19, I-20, 1-21, 1-22, 1-23, 1-24, I-25, 1-26, 1-27, 1-28, 1-29, I-30, 1-31, 1-32, 1-33, 1-34, I-36, 1-37, I-38, 1-39, 1-40, 1-41, 1-42, I-43, 1-44, 1-45, 1-46, 1-49, I-51, 1-52, 1-58, 1-60, 1-64, I-65, 1-67, I-74, 1-75, 1-76, 1-77, 1-78, I-79, 1-80, 1-81, 1-82, 1-83, I-84, 1-85, 1-86, 1-87, 1-91, I-93, 1-94, I-95, 1-96, 1-97, 1-98, 1-99, I-100, 1-101, 1-102, and 1-103. Starting materials used when synthesizing these compounds are listed in Table 3.

TABLE 3

| Compound | | |
|---|---|---|
| Product | Starting Materials | |
| I-2 | 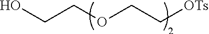 | |
| I-3 | 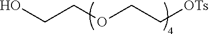 | |
| I-4 | 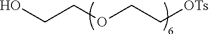 | |
| I-5 | 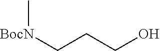 | |
| I-6 | 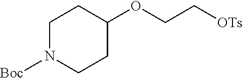 [SMILES: Cc1ccc(S(=O)(=O)OCCOC2CCN(C(=O)OC(C)(C)C)CC2)cc1] | |
| I-7 | 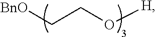 | 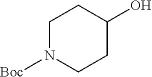 [SMILES: CC(C)(C)OC(=O)N1CCC(O)CC1] |

| I-8 | 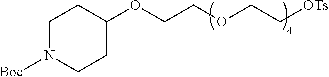 | |
|---|---|---|
| I-9 | 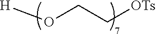 | |
| I-10 | 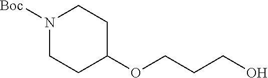 [SMILES: CC(C)(C)OC(=O)N1CCC(OCCCO)CC1] | |
| I-11 | 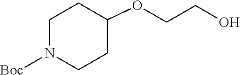 [SMILES: CC(C)(C)OC(=O)N1CCC(OCCO)CC1] | |
| I-12 | 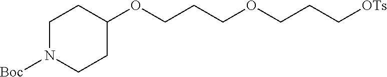 [SMILES: Cc1ccc(S(=O)(=O)OCCCOCCCOC2CCN(C(=O)OC(C)(C)C)CC2)cc1] | |
| I-13 | 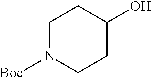 [SMILES: CC(C)(C)OC(=O)N1CCC(O)CC1] | |
| I-14 | 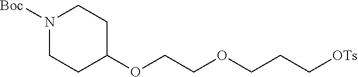 [SMILES: Cc1ccc(S(=O)(=O)OCCCOCCOC2CCN(C(=O)OC(C)(C)C)CC2)cc1] | |
| I-15 | 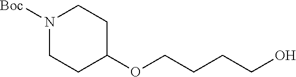 [SMILES: CC(C)(C)OC(=O)N1CCC(OCCCCO)CC1] | |
| I-16 | 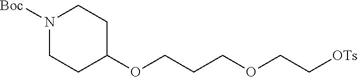 [SMILES: Cc1ccc(S(=O)(=O)OCCOCCCOC2CCN(C(=O)OC(C)(C)C)CC2)cc1] | |
| I-17 | 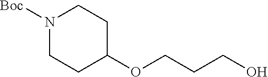 [SMILES: CC(C)(C)OC(=O)N1CCC(OCCCO)CC1] | |
| I-19 | 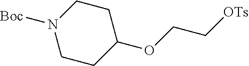 [SMILES: Cc1ccc(S(=O)(=O)OCCOC2CCN(C(=O)OC(C)(C)C)CC2)cc1] | |
| I-20 | 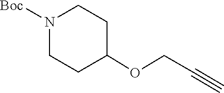 [SMILES: C#CCOC1CCN(C(=O)OC(C)(C)C)CC1] | |
| I-21 | 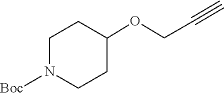 [SMILES: C#CCOC1CCN(C(=O)OC(C)(C)C)CC1] | |
| I-22 | 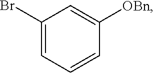 [SMILES: Brc1cccc(OCc2ccccc2)c1] | 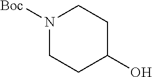 [SMILES: CC(C)(C)OC(=O)N1CCC(O)CC1] |
| I-23 | 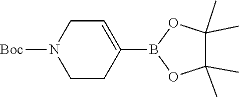 [SMILES: CC(C)(C)OC(=O)N1CC=C(B2OC(C)(C)C(C)(C)O2)CC1] | |
| I-24 | 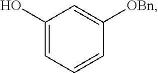 [SMILES: Oc1cccc(OCc2ccccc2)c1] | 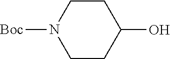 [SMILES: CC(C)(C)OC(=O)N1CCC(O)CC1] |
| I-25 | 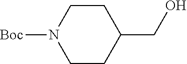 [SMILES: CC(C)(C)OC(=O)N1CCC(CO)CC1] | |
| I-26 | 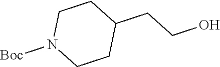 [SMILES: CC(C)(C)OC(=O)N1CCC(CCO)CC1] | |
| I-27 | 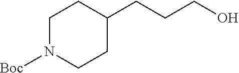 [SMILES: CC(C)(C)OC(=O)N1CCC(CCCO)CC1] | |
| I-28 | 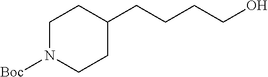 [SMILES: CC(C)(C)OC(=O)N1CCC(CCCCO)CC1] | |
| I-29 | 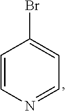 | 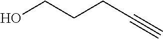 |
| I-30 | 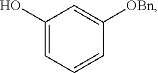 [SMILES: Oc1cccc(OCc2ccccc2)c1] | 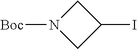 [SMILES: CC(C)(C)OC(=O)N1CC(I)C1] |
| I-31 | 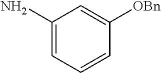 [SMILES: Nc1cccc(OCc2ccccc2)c1] | |
| I-32 | 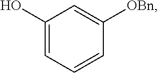 [SMILES: Oc1cccc(OCc2ccccc2)c1] | 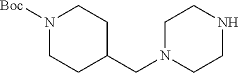 [SMILES: CC(C)(C)OC(=O)N1CCC(CN2CCNCC2)CC1] |

| | | |
|---|---|---|
| I-33 | 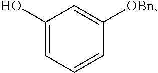 [SMILES: Oc1cccc(OCc2ccccc2)c1] | 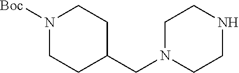 [SMILES: CC(C)(C)OC(=O)N1CCC(CN2CCNCC2)CC1] |
| I-34 | 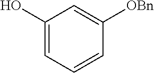 [SMILES: Oc1cccc(OCc2ccccc2)c1] | 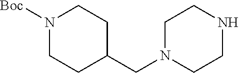 [SMILES: CC(C)(C)OC(=O)N1CCC(CN2CCNCC2)CC1] |

| I-36 | 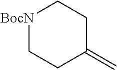 | |
|---|---|---|
| I-37 | 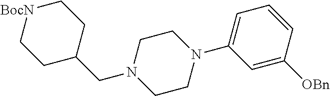 [SMILES: CC(C)(C)OC(=O)N1CCC(CN2CCN(c3cccc(OCc4ccccc4)c3)CC2)CC1] | |
| I-38 | 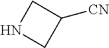 | |
| I-39 | 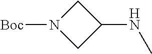 | |
| I-40 | 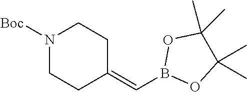 [SMILES: CC(C)(C)OC(=O)N1CCC(=CB2OC(C)(C)C(C)(C)O2)CC1] | |
| I-41 | 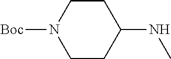 | |
| I-42 | 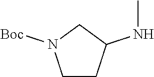 | |
| I-43 | 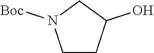 [SMILES: CC(C)(C)OC(=O)N1CCC(O)C1] | |
| I-44 | 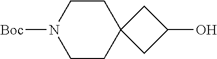 [SMILES: CC(C)(C)OC(=O)N1CCC2(CC1)CC(O)C2] | |
| I-45 | 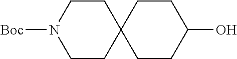 [SMILES: CC(C)(C)OC(=O)N1CCC2(CCC(O)CC2)CC1] | |
| I-46 | 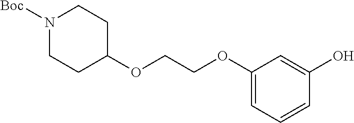 [SMILES: CC(C)(C)OC(=O)N1CCC(OCCOc2cccc(O)c2)CC1] | |
| I-49 | 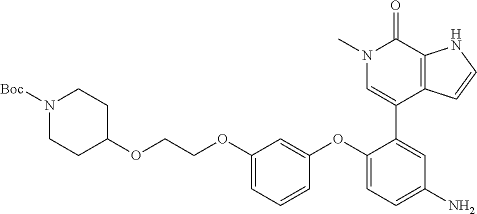 [SMILES: Cn1cc(-c2cc(N)ccc2Oc2cccc(OCCOC3CCN(C(=O)OC(C)(C)C)CC3)c2)c2cc[nH]c2c1=O] | |
| I-51 | 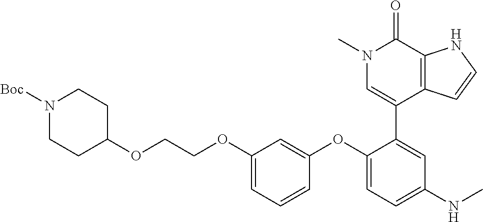 [SMILES: CNc1ccc(Oc2cccc(OCCOC3CCN(C(=O)OC(C)(C)C)CC3)c2)c(-c2cn(C)c(=O)c3[nH]ccc23)c1] | |
| I-52 | 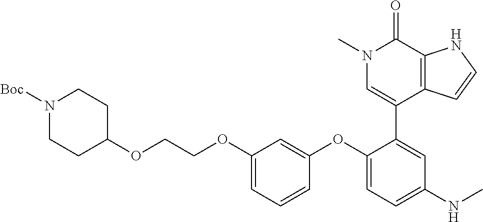 [SMILES: CNc1ccc(Oc2cccc(OCCOC3CCN(C(=O)OC(C)(C)C)CC3)c2)c(-c2cn(C)c(=O)c3[nH]ccc23)c1] | |
| I-58 | 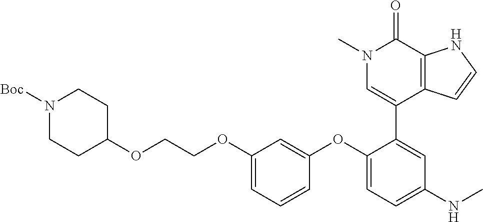 [SMILES: CNc1ccc(Oc2cccc(OCCOC3CCN(C(=O)OC(C)(C)C)CC3)c2)c(-c2cn(C)c(=O)c3[nH]ccc23)c1] | |
| I-60 | 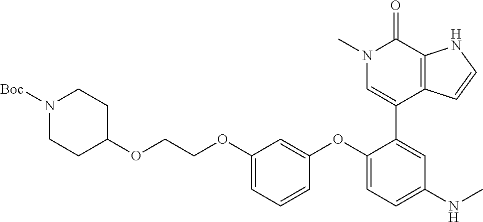 [SMILES: CNc1ccc(Oc2cccc(OCCOC3CCN(C(=O)OC(C)(C)C)CC3)c2)c(-c2cn(C)c(=O)c3[nH]ccc23)c1] | |
| I-64 | 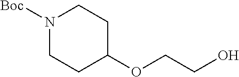 [SMILES: CC(C)(C)OC(=O)N1CCC(OCCO)CC1] | |
| I-65 | 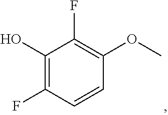 | 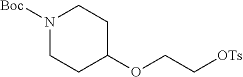 [SMILES: Cc1ccc(S(=O)(=O)OCCOC2CCN(C(=O)OC(C)(C)C)CC2)cc1] |

| I-67 | 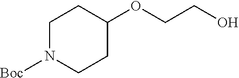 [SMILES: CC(C)(C)OC(=O)N1CCC(OCCO)CC1] | |
|---|---|---|
| I-74 | 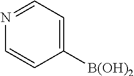 | |
| I-75 | 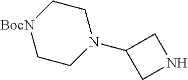 [SMILES: CC(C)(C)OC(=O)N1CCN(C2CNC2)CC1] | |
| I-76 | 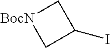 [SMILES: CC(C)(C)OC(=O)N1CC(I)C1] | |
| I-77 | 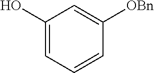 [SMILES: Oc1cccc(OCc2ccccc2)c1] | 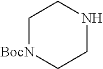 [SMILES: CC(C)(C)OC(=O)N1CCNCC1] |
| I-78 | 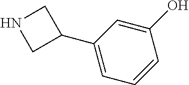 [SMILES: Oc1cccc(C2CNC2)c1] | |
| I-79 | 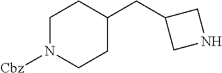 [SMILES: O=C(OCc1ccccc1)N1CCC(CC2CNC2)CC1] | |
| I-80 | 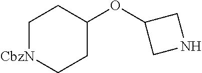 [SMILES: O=C(OCc1ccccc1)N1CCC(OC2CNC2)CC1] | |
| I-81 | 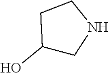 | |
| I-82 | 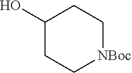 [SMILES: CC(C)(C)OC(=O)N1CCC(O)CC1] | |
| I-83 | 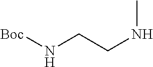 | |
| I-84 | 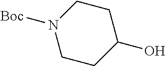 [SMILES: CC(C)(C)OC(=O)N1CCC(O)CC1] | |
| I-85 | 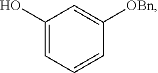 [SMILES: Oc1cccc(OCc2ccccc2)c1] | 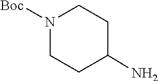 [SMILES: CC(C)(C)OC(=O)N1CCC(N)CC1] |

| | |
|---|---|
| I-86 | 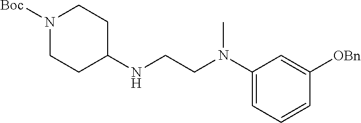 [SMILES: CN(CCNC1CCN(C(=O)OC(C)(C)C)CC1)c1cccc(OCc2ccccc2)c1] |
| I-87 | 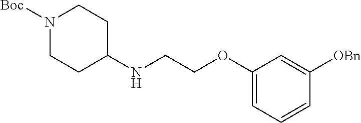 [SMILES: CC(C)(C)OC(=O)N1CCC(NCCOc2cccc(OCc3ccccc3)c2)CC1] |
| I-91 | 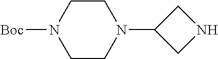 [SMILES: CC(C)(C)OC(=O)N1CCN(C2CNC2)CC1] |
| I-93 | 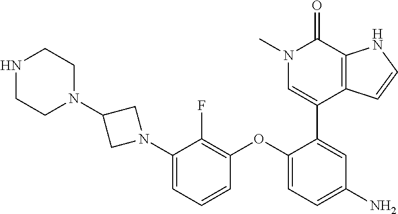 [SMILES: Cn1cc(-c2cc(N)ccc2Oc2cccc(N3CC(N4CCNCC4)C3)c2F)c2cc[nH]c2c1=O] |
| I-94 | 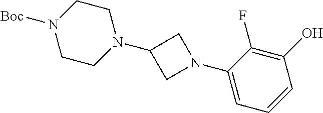 [SMILES: CC(C)(C)OC(=O)N1CCN(C2CN(c3cccc(O)c3F)C2)CC1] |
| I-95 | 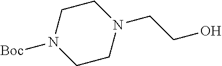 [SMILES: CC(C)(C)OC(=O)N1CCN(CCO)CC1] |
| I-96 | 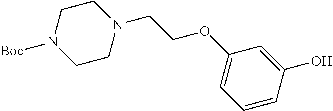 [SMILES: CC(C)(C)OC(=O)N1CCN(CCOc2cccc(O)c2)CC1] |
| I-97 | 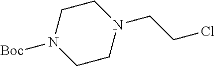 [SMILES: CC(C)(C)OC(=O)N1CCN(CCCl)CC1] |
| I-98 | 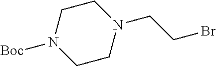 [SMILES: CC(C)(C)OC(=O)N1CCN(CCBr)CC1] |
| I-99 | 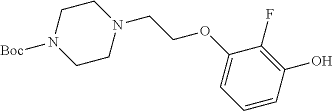 [SMILES: CC(C)(C)OC(=O)N1CCN(CCOc2cccc(O)c2F)CC1] |
| I-100 | 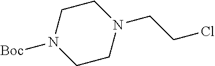 [SMILES: CC(C)(C)OC(=O)N1CCN(CCCl)CC1] |
| I-101 | 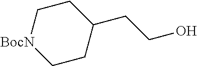 [SMILES: CC(C)(C)OC(=O)N1CCC(CCO)CC1] |
| I-102 | 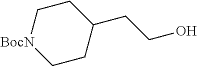 [SMILES: CC(C)(C)OC(=O)N1CCC(CCO)CC1] |
| I-103 | 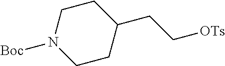 [SMILES: Cc1ccc(S(=O)(=O)OCCC2CCN(C(=O)OC(C)(C)C)CC2)cc1] |

### Example 29—Synthesis of N-[3-(3-chloro-4-cyano-phenoxy)-2,2,4,4-tetramethyl-cyclobutyl]-2-[4-[3-[3-[2-(6-methyl-7-oxo-1H-pyrrolo[2,3-c]pyridin-4-yl)phenoxy]phenyl]propyl]piperazin-1-yl]pyrimidine-5-carboxamide (I-134)

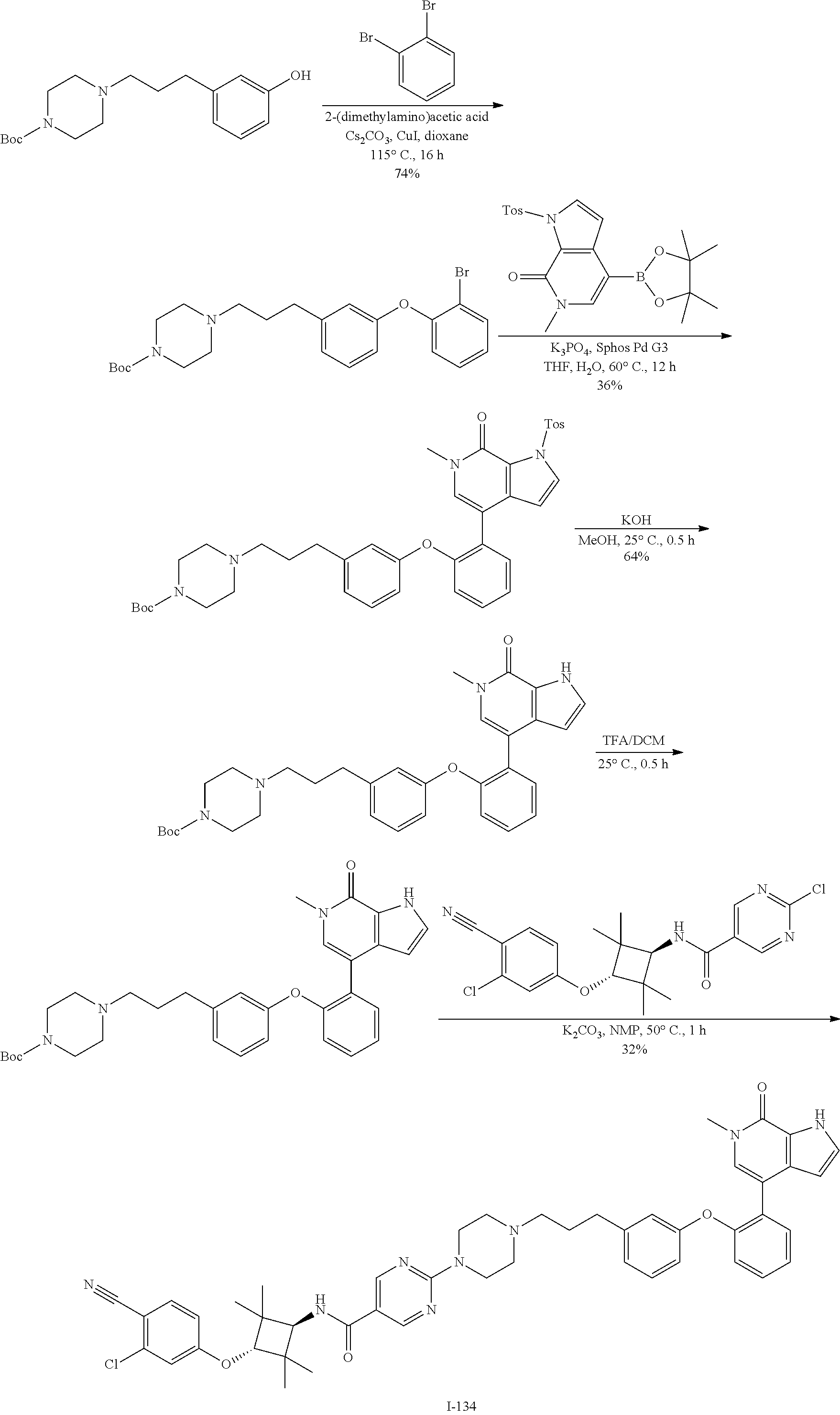

Step 1: Preparation of tert-butyl 4-[3-[3-(2-bromophenoxy)phenyl]propyl]piperazine-1-carboxylate. To a solution of tert-butyl 4-[3-(3-hydroxyphenyl) propyl]piperazine-1-carboxylate (500 mg, 1.56 mmol, 1.0 equiv) and 1,2-dibromobenzene (1.10 g. 4.68 mmol, 3.0 equiv) in dioxane (10 mL) was added Cs₂CO₃ (1.53 g, 4.68 mmol, 3.0 equiv) and 2-(dimethylamino)acetic acid (193 mg, 1.87 mmol, 1.2 equiv) and CuI (118 mg, 624 umol. 0.4 equiv) at 25° C. The mixture was stirred at 115° C. for 16 h and concentrated. The residue was purified by column chromatography (SiO₂, petroleum ether/ethyl acetate=20:1 to 1:1) to give tert-butyl 4-[3-[3-(2-bromophenoxy)phenyl]propyl]piperazine-1-carboxylate (550 mg, 74% yield) was obtained as a yellow oil.

Step 2: Preparation of tert-butyl 4-[3-[3-[2-[6-methyl-7-oxo-1-(p-tolylsulfonyl)pyrrolo[2,3-c]pyridin-4-yl]phenoxy]phenyl]propyl]piperazine-1-carboxylate. To a solution of tert-butyl 4-[3-[3-(2-bromophenoxy)phenyl]propyl]piperazine-1-carboxylate (400 mg, 841 umol, 1.0 equiv) and 6-methyl-1-(p-tolylsulfonyl)-4-(4,4,5,5-tetramethyl-1,3,2-dioxaborolan-2-yl)pyrrolo[2,3-c]pyridin-7-one (432 mg, 1.01 mmol, 1.2 equiv) in THF (5 mL) and H₂O (1 mL) was added SPhos Pd G₃ (65 mg, 84 umol, 0.1 equiv) and K₃PO₄ (535 mg, 2.52 mmol, 3.0 equiv). The mixture was stirred at 60° C. for 12 h and concentrated. To the reaction mixture was added water (10 mL) and the mixture was extracted with EtOAc (30 mL). The combined organic phase was washed with brine (3×10 mL), dried over anhydrous Na₂SO₄, filtered and concentrated in vacuum. The residue was purified by column chromatography (SiO₂, petroleum ether/ethyl acetate=5:1 to 0:1) to give tert-butyl 4-[3-[3-[2-[6-methyl-7-oxo-1-(p-tolylsulfonyl)pyrrolo[2,3-c]pyridin-4-yl]phenoxy]phenyl]propyl]piperazine-1-carboxylate (210 mg, 36% yield) was obtained as a colorless oil.

Step 3: Preparation of tert-butyl 4-[3-[3-[2-(6-methyl-7-oxo-1H-pyrrolo[2,3-c]pyridin-4-yl)phenoxy]phenyl]propyl]piperazine-1-carboxylate. To a solution of tert-butyl 4-[3-[3-[2-[6-methyl-7-oxo-1-(p-tolylsulfonyl)pyrrolo[2,3-c]pyridin-4-yl]phenoxy]phenyl]propyl]piperazine-1-carboxylate (200 mg, 287 umol, 1.0 equiv) in MeOH (3 mL) was added KOH (48 mg, 0.86 mmol, 3.0 equiv). The mixture was stirred at 25° C. for 0.5 h and concentrated. The residue was purified by prep-HPLC (column: Unisil 3-100 C18 Ultra 150 mm*50 mm*3 μm;mobile phase: [water(FA)-ACN];B %: 21%-51%, 7 min) to give tert-butyl 4-[3-[3-[2-(6-methyl-7-oxo-1H-pyrrolo[2,3-c]pyridin-4-yl)phenoxy]phenyl]propyl]piperazine-1-carboxylate (100 mg, 64% yield) was obtained as a white solid.

Step 4: Preparation of 6-methyl-4-[2-[3-(3-piperazin-1-ylpropyl)phenoxy]phenyl]-1H-pyrrolo[2,3-c]pyridin-7-one. To a solution of tert-butyl 4-[3-[3-[2-(6-methyl-7-oxo-1H-pyrrolo[2,3-c]pyridin-4-yl)phenoxy]phenyl]propyl]piperazine-1-carboxylate (50 mg, 92 mol, 1 equiv) in DCM (4 mL) was added TFA (2 mL). The mixture was stirred at 25° C. for 1 h. Without purification, it was used for the next step directly.

Step 5: Preparation of N-[3-(3-chloro-4-cyano-phenoxy)-2,2,4,4-tetramethyl-cyclobutyl]-2-[4-[3-[3-[2-(6-methyl-7-oxo-1H-pyrrolo[2,3-c]pyridin-4-yl)phenoxy]phenyl]propyl]piperazin-1-yl]pyrimidine-5-carboxamide. To a solution of 6-methyl-4-[2-[3-(3-piperazin-1-ylpropyl)phenoxy]phenyl]-1H-pyrrolo[2,3-c]pyridin-7-one (40 mg, 71 mol, 1.0 equiv, TFA salt) and 2-chloro-N-[3-(3-chloro-4-cyano-phenoxy)-2,2,4,4-tetramethyl-cyclobutyl]pyrimidine-5-carboxamide (36 mg, 86 μmol, 1.2 equiv) in NMP (2 mL) was added K₂CO₃ (30 mg, 0.22 mmol, 3.0 equiv). The mixture was stirred at 50° C. for 1 h and concentrated. The residue was purified by prep-HPLC (column: Unisil 3-100 C18 Ultra 150 mm*50 mm*3 um; mobile phase: [water(FA)-ACN];B %: 35%-65%, 7 min) to give N-[3-(3-chloro-4-cyano-phenoxy)-2,2,4,4-tetramethyl-cyclobutyl]-2-[4-[3-[3-[2-(6-methyl-7-oxo-1H-pyrrolo [2,3-c]pyridin-4-yl)phenoxy]phenyl]propyl]piperazin-1-yl]pyrimidine-5-carboxamide (19 mg, 32% yield, 99.14% purity) was obtained as a white solid. ¹H NMR (400 MHz, DMSO-d₆): δ 11.98 (s, 1H), 8.77 (s, 2H), 7.89 (d, J=8.76 Hz, 1H), 7.73 (d, J=8.40 Hz, 1H), 7.49 (d, J=7.56, 1.64 Hz, 1H), 7.37 (d, J=7.76, 1.76 Hz, 1H), 7.23-7.27 (m, 2H), 7.22 (s, 1H), 7.15-7.21 (m, 2H), 6.98-7.01 (m, 2H), 6.87 (d, J=7.56 Hz, 1H), 6.67-6.73 (m, 2H), 6.19-6.22 (m, 1H), 4.27-4.28 (m, 1H), 4.01-4.04 (m, 1H), 3.66-3.88 (m, 3H), 3.31 (s, 4H), 2.53 (s, 1H), 2.51 (d, J=1.88 Hz, 2H), 2.20-2.44 (m, 4H), 1.60-1.76 (m, 2H), 1.20 (s, 6H), 1.10 (s, 6H), 1.08 (s, 1H). LC-MS: MS (ES⁺): RT=2.491 min, m/z=825.3 [M/2+H+]; LCMS method: 25.

### Example 30—Synthesis of N-[3-(3-chloro-4-cyano-phenoxy)-2,2,4,4-tetramethyl-cyclobutyl]-2-[4-[2-[3-[4-isopropyl-2-(6-methyl-7-oxo-1H-pyrrolo[2,3-c]pyridin-4-yl)phenoxy]phenoxy]ethyl]piperazin-1-yl]pyrimidine-5-carboxamide (I-107)

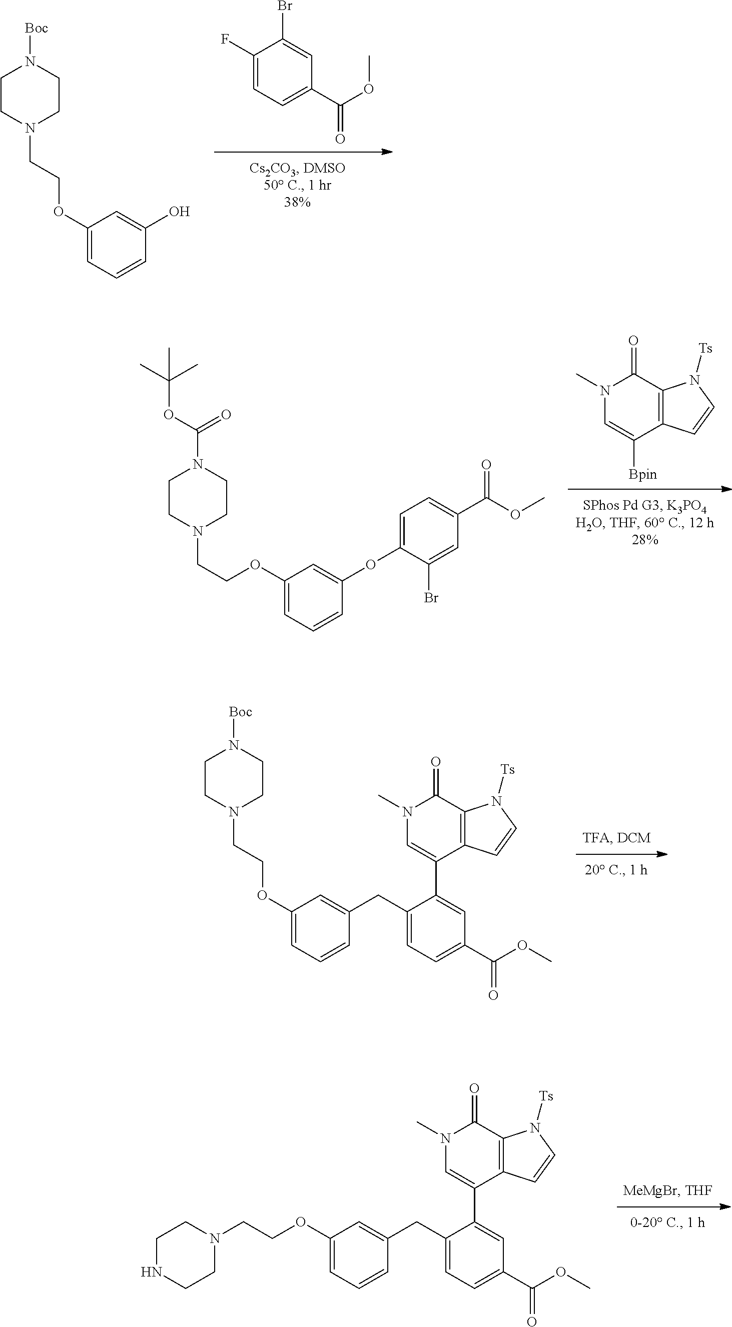

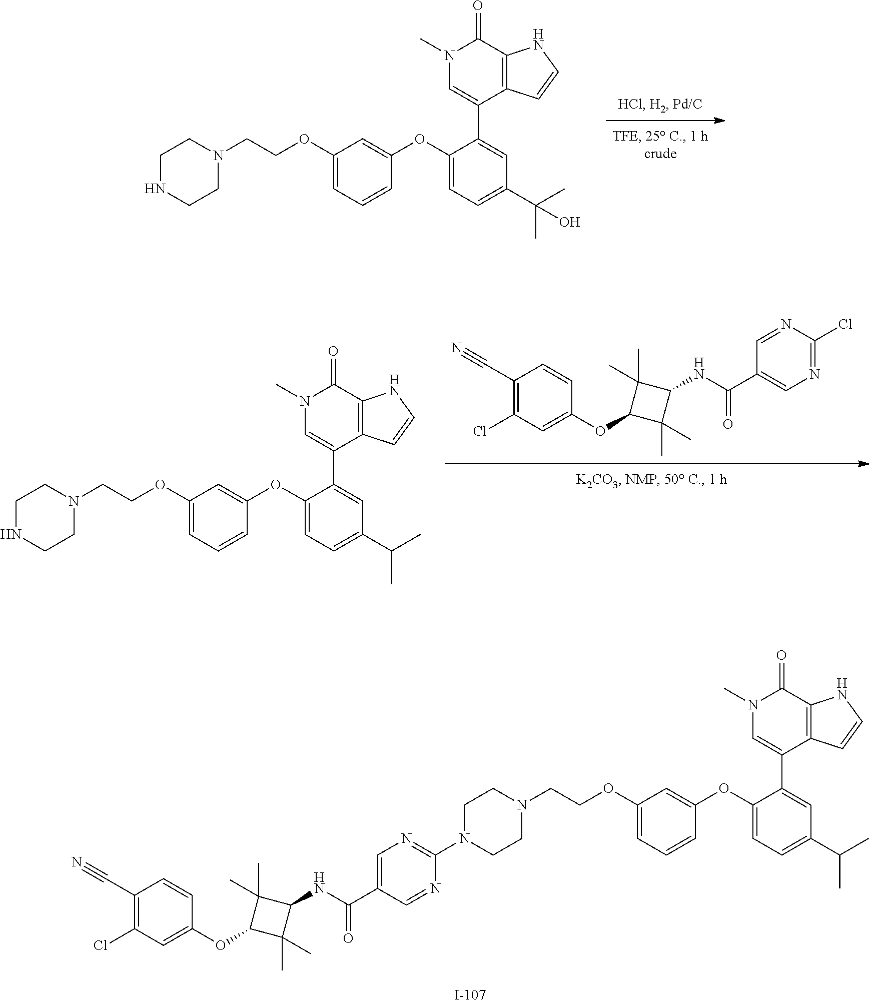 [SMILES: Cn1cc(-c2cc(C(C)(C)O)ccc2Oc2cccc(OCCN3CCNCC3)c2)c2cc[nH]c2c1=O ; CC(C)c1ccc(Oc2cccc(OCCN3CCNCC3)c2)c(-c2cn(C)c(=O)c3[nH]ccc23)c1 ; CC1(C)[C@H](NC(=O)c2cnc(Cl)nc2)C(C)(C)[C@H]1Oc1ccc(C#N)c(Cl)c1 ; CC(C)c1ccc(Oc2cccc(OCCN3CCN(c4ncc(C(=O)N[C@H]5C(C)(C)[C@H](Oc6ccc(C#N)c(Cl)c6)C5(C)C)cn4)CC3)c2)c(-c2cn(C)c(=O)c3[nH]ccc23)c1]

Step 1: Preparation of tert-butyl 4-[2-[3-(2-bromo-4-methoxycarbonyl-phenoxy)phenoxy]ethyl]piperazine-1-carboxylate. To a solution of tert-butyl 4-[2-(3-hydroxyphenoxy)ethyl]piperazine-1-carboxylate (1 g, 3.10 mmol, 1 equiv) and methyl 3-bromo-4-fluoro-benzoate (722 mg, 3.1 mmol, 1 equiv) in DMSO (8 mL) was added Cs₂CO₃ (2.53 g, 7.75 mmol, 2.5 equiv). The mixture was stirred at 50° C. for 1 h and quenched by addition 30 mL water. The mixture was extracted with EA (3×50 mL). The combined organic layers were washed with brine 100 mL, dried over anhydrous sodium sulfate, filtered, and concentrated. The residue was purified by prep-HPLC (column: Waters Xbridge C18 150 mm *50 mm* 10 μm; mobile phase: [water (NH₄HCO₃)-ACN]; B %: 57%-87%, 10 min) to give tert-butyl 4-[2-[3-(2-bromo-4-methoxycarbonyl-phenoxy)phenoxy]ethyl]piperazine-1-carboxylate (630 mg. 38% yield) as a yellow oil.

Step 2: Preparation of tert-butyl 4-[2-[3-[4-methoxycarbonyl-2-[6-methyl-7-oxo-1-(p-tolylsulfonyl)pyrrolo[2,3-c]pyridin-4-yl]phenoxy]phenoxy]ethyl]piperazine-1-carboxylate. To a solution of tert-butyl 4-[2-[3-(2-bromo-4-methoxycarbonyl-phenoxy)phenoxy]ethyl]piperazine-1-carboxylate (620 mg, 1.16 mmol, 1 equiv) and 6-methyl-1-(p-tolylsulfonyl)-4-(4,4,5,5-tetramethyl-1,3,2-dioxaborolan-2-yl)pyrrolo[2,3-c]pyridin-7-one (595 mg, 1.4 mmol, 1.2 equiv) in H₂O (3 mL) and THF (15 mL) was added SPhos Pd G3 (90 mg, 115 mol, 0.1 equiv) and K₃PO₄ (737 mg, 3.5 mmol, 3 equiv). The mixture was stirred at 60° C. for 12 h under N₂. The residue was purified by prep-HPLC (column: Phenomenex luna C18 150 mm*40 mm*15 μm; mobile phase: [water(FA)-ACN]; B %: 28%-58%, 10 min) to give tert-butyl 4-[2-[3-[4-methoxycarbonyl-2-[6-methyl-7-oxo-1-(p-tolylsulfonyl)pyrrolo[2,3-c]pyridin-4-yl]phenoxy]phenoxy]ethyl]piperazine-1-carboxylate (250 mg, 28% yield) as a yellow oil.

Step 3: Preparation of 3-[6-methyl-7-oxo-1-(p-tolylsulfonyl)pyrrolo[2,3-c]pyridin-4-yl]-4-[3-(2-piperazin-1-ylethoxy)phenoxylbenzoate. To a solution of tert-butyl 4-[2-[3-[4-methoxycarbonyl-2-[6-methyl-7-oxo-1-(p-tolylsulfonyl)pyrrolo[2,3-c]pyridin-4-yl]phenoxy]phenoxy]ethyl]piperazine-1-carboxylate (230 mg, 303 μmol, 1 equiv) in DCM (2 mL) was added TFA (1 mL). The mixture was stirred at 20° C. for 1 h and concentrated to give methyl 3-[6-methyl-7-oxo-1-(p-tolylsulfonyl)pyrrolo[2,3-c]pyridin-4-yl]-4-[3-(2-piperazin-1-ylethoxy)phenoxy]benzoate (230 mg, crude, TFA).

Step 4: Preparation of 4-[5-(1-hydroxy-1-methyl-ethyl)-2-[3-(2-piperazin-1-ylethoxy)phenoxy]phenyl]-6-methyl-1-(p-tolylsulfonyl)pyrrolo[2,3-c]pyridin-7-one. To a solution of methyl 3-[6-methyl-7-oxo-1-(p-tolylsulfonyl)pyrrolo[2,3-c]pyridin-4-yl]-4-[3-(2-piperazin-1-ylethoxy)phenoxy]benzoate (200 mg, 259 μmol, 1 equiv, TFA salt) in THF (5 mL) was added MeMgBr (3 M, 432 μL, 5 equiv) at 0° C. The mixture was stirred at 20° C. for 1 h and quenched by addition 30 mL water. The mixture was extracted with EA (3×50 mL). The combined organic layers were washed with brine 100 mL, dried over anhydrous sodium sulfate, filtered and concentrated to give 4-[5-(1-hydroxy-1-methyl-ethyl)-2-[3-(2-piperazin-1-ylethoxy)phenoxy]phenyl]-6-methyl-1-(p-tolylsulfonyl)pyrrolo[2,3-c]pyridin-7-one (150 mg, crude) as a yellow oil.

Step 5: Preparation of 4-[5-isopropyl-2-[3-(2-piperazin-1-ylethoxy) phenoxy]phenyl]-6-methyl-1H-pyrrolo [2, 3-cl pyridin-7-one. To a solution of 4-[5-(1-hydroxy-1-methyl-ethyl)-2-[3-(2-piperazin-1-ylethoxy)phenoxy]phenyl]-6-methyl-1H-pyrrolo[2,3-c]pyridin-7-one (53 mg, 1 eq, TFA) and HCl (12 M, 7 μL, 1 equiv) in TFE (2 mL) was added Pd/C (20 mg, 10% purity) under N₂ atmosphere. The mixture was stirred under H₂ (15 psi.) at 25° C. for 1 h. The mixture was filtered and concentrated to give 4-[5-isopropyl-2-[3-(2-piperazin-1-ylethoxy) phenoxy]phenyl]-6-methyl-1H-pyrrolo [2, 3-c]pyridin-7-one (15 mg, crude).

Step 6: Preparation of N-[3-(3-chloro-4-cyano-phenoxy)-2,2,4,4-tetramethyl-cyclobutyl]-2-[4-[2-[3-[4-isopropyl-2-(6-methyl-7-oxo-1H-pyrrolo[2,3-c]pyridin-4-yl)phenoxy]phenoxy]ethyl]piperazin-1-yl]pyrimidine-5-carboxamide. To a solution of 4-[5-isopropyl-2-[3-(2-piperazin-1-ylethoxy)phenoxy]phenyl]-6-methyl-1H-pyrrolo[2,3-c]pyridin-7-one (10 mg, 20.5 mol, 1 equiv) and 2-chloro-N-[3-(3-chloro-4-cyano-phenoxy)-2,2,4,4-tetramethyl-cyclobutyl]pyrimidine-5-carboxamide (8.6 mg, 20 μmol, 1 equiv) in NMP (2 mL) was added K₂CO₃ (8.5 mg, 61 mol, 3 equiv). The mixture was stirred at 50° C. for 1 h and purified by prep-HPLC (column: Unisil 3-100 C18 Ultra 150*50 mm*3 μm;mobile phase: [water(FA)-ACN];B %: 31%-61%, 7 min) to give N-[3-(3-chloro-4-cyano-phenoxy)-2,2,4,4-tetramethyl-cyclobutyl]-2-[4-[2-[3-[4-isopropyl-2-(6-methyl-7-oxo-1H-pyrrolo[2,3-c]pyridin-4-yl)phenoxy]phenoxy]ethyl]piperazin-1-yl]pyrimidine-5-carboxamide (2 mg, 11% yield). 1H NMR (400 MHz, MeOD) δ 8.79-8.73 (m, 2H), 8.53-8.41 (m, 1H), 7.75-7.69 (m, 1H), 7.42-7.39 (m, 1H), 7.32-7.28 (m, 2H), 7.15-7.12 (m, 2H), 7.08-6.95 (m, 3H), 6.54-6.48 (m, 1H), 6.36-6.28 (m, 3H), 4.29 (s, 1H), 4.16-4.11 (m, 1H), 4.03-3.97 (m, 2H), 3.97-3.90 (m, 4H), 3.60-3.57 (m, 3H), 3.04 (s, 1H), 2.80-2.76 (m, 2H), 2.65-2.59 (m, 4H), 1.32 (d, J=7.4 Hz, 6H), 1.28 (s, 6H), 1.21 (s, 6H). LC-MS: MS (ES⁺): RT=2.196 min, m/z=869.3 [M+H⁺]; LCMS Method: 25.

### Example 31—Synthesis of N-[3-(3-chloro-4-cyano-phenoxy)-2,2,4,4-tetramethyl-cyclobutyl]-2-[4-[2-[[1-[4-(ethylsulfonylamino)-2-(6-methyl-7-oxo-1H-pyrrolo[2,3-c]pyridin-4-yl)phenyl]-2-oxo-4-pyridyl]oxy]ethoxy]-1-piperidyl]pyrimidine-5-carboxamide (I-112)

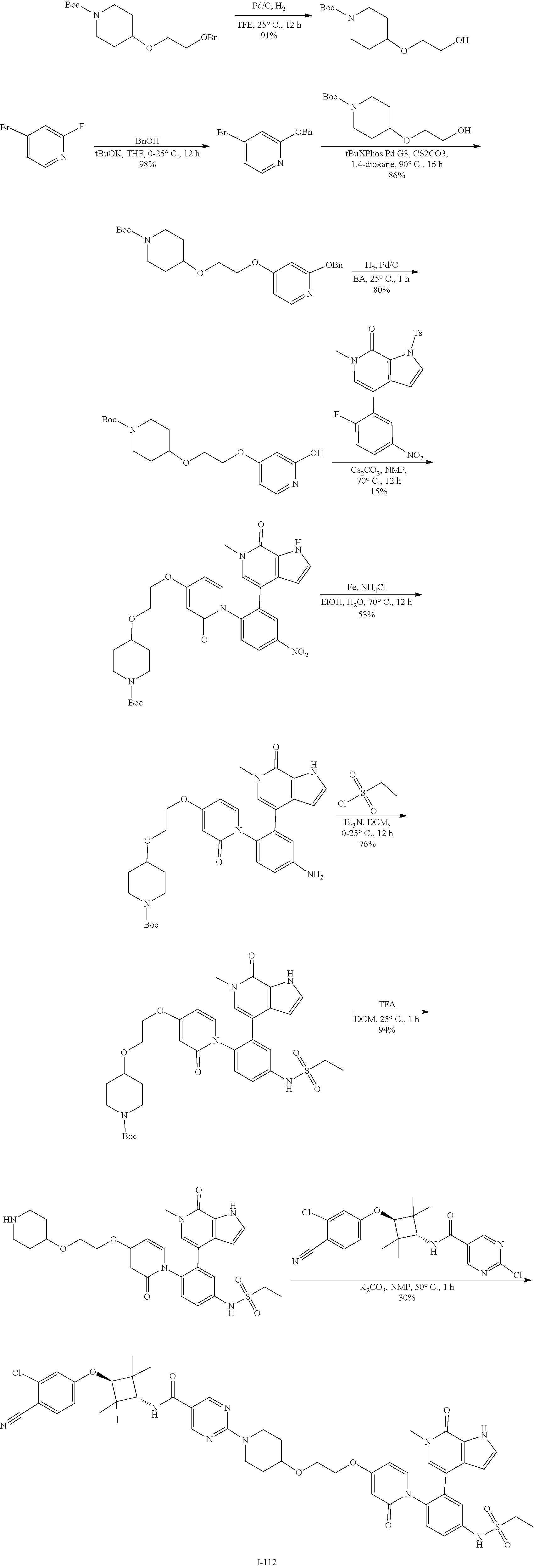

Step 1: Preparation of tert-butyl 4-(2-hydroxyethoxy)piperidine-1-carboxylate. To a solution of tert-butyl 4-(2-benzyloxyethoxy)piperidine-1-carboxylate (3.0 g, 8.9 mmol, 1.0 equiv) in TFE (20 mL) was added Pd/C (1.15 g, 8.94 mmol, 10% purity, 1.0 equiv) under N₂ atmosphere. The suspension was degassed and purged with H₂ three times. The mixture was stirred under H₂ (15 psi) at 25° C. for 12 h. The reaction mixture was filtered and concentrated in vacuo to give the crude product. The crude product tert-butyl 4-(2-hydroxyethoxy)piperidine-1-carboxylate (2.0 g, 8.2 mmol, 91% yield) was a colorless oil used into the next step without further purification.

Step 2: Preparation of 2-benzyloxy-4-bromo-pyridine. To a solution of 4-bromo-2-fluoro-pyridine (3.0 g, 17 mmol, 1.0 equiv) and phenylmethanol (1.84 g, 17.1 mmol, 1.77 mL, 1.0 equiv) in THF (20 mL) was added dropwise tBuOK (1 M, 17 mL, 1.0 equiv) at 0° C. The resulting mixture was stirred at 25° C. for 1 h. The reaction mixture was filtered and concentrated in vacuo to give the crude product. The crude product was purified by column chromatography on silica gel (petroleum ether: ethyl acetate=10:1 to 5:1). The compound 2-benzyloxy-4-bromo-pyridine (4.40 g, 16.7 mmol, 98% yield) was obtained as a colorless oil.

Step 3: Preparation of tert-butyl 4-[2-[(2-benzyloxy-4-pyridyl)oxy]ethoxy]piperidine-1-carboxylate. To a solution of tert-butyl 4-(2-hydroxyethoxy)piperidine-1-carboxylate (1 g, 4 mmol, 2.0 equiv) in 1,4-dioxane (2 mL) was added Cs₂CO₃ (2.66 g, 8.15 mmol, 3.0 equiv) tBuXPhos Pd G3 (215.8 mg, 271.8 mol, 0.10 equiv) and tBuXPhos Pd G3 (215.9 mg, 271.8 mol, 0.10 equiv) and 2-benzyloxy-4-bromo-pyridine (718 mg, 2.70 mmol, 1.0 equiv). The mixture was stirred at 90° C. for 16 h. The reaction mixture was filtered and concentrated in vacuo to give the crude product. The crude product was purified by column chromatography on silica gel (petroleum ether: ethyl acetate=10:1 to 1:1). The compound tert-butyl 4-[2-[(2-benzyloxy-4-pyridyl)oxy]ethoxy]piperidine-1-carboxylate (1.0 g, 2.3 mmol, 86% yield) was obtained a yellow oil.

Step 4: Preparation of tert-butyl 4-[2-[(2-hydroxy-4-pyridyl)oxy]ethoxy]piperidine-1-carboxylate. To a solution of tert-butyl 4-[2-[(2-benzyloxy-4-pyridyl)oxy]ethoxy]piperidine-1-carboxylate (1.0 g, 2.3 mmol, 1.0 equiv) in EtOAc (5 mL) was added Pd/C (299 mg, 2.33 mmol, 10% purity, 1.0 equiv). The suspension was degassed and purged with H₂ three times. The mixture was stirred under H₂ (15 psi) at 25° C. for 1 h. The reaction mixture was filtered and concentrated in vacuo to give the crude product. The crude product tert-butyl 4-[2-[(2-hydroxy-4-pyridyl)oxy]ethoxy]piperidine-1-carboxylate (630 mg, 1.86 mmol, 80% yield) was obtained as a yellow oil and used int the next step without further purification.

Step 5: Preparation of tert-butyl 4-[2-[[1-[2-(6-methyl-7-oxo-1H-pyrrolo[2,3-c]pyridin-4-yl)-4-nitro-phenyl]-2-oxo-4-pyridyl]oxy]ethoxy]piperidine-1-carboxylate. A mixture of tert-butyl 4-[2-[(2-hydroxy-4-pyridyl)oxy]ethoxy]piperidine-1-carboxylate (530 mg, 1.57 mmol, 1.0 equiv), 4-(2-fluoro-5-nitro-phenyl)-6-methyl-1-(p-tolylsulfonyl) pyrrolo[2,3-c]pyridin-7-one (691 mg, 1.57 mmol, 1.0 equiv), Cs₂CO₃ (1.5 g, 4.7 mmol, 3.0 equiv) in NMP (8 mL) was degassed and purged with N₂ three times, and then the mixture was stirred at 70° C. for 12 h under N₂ atmosphere. The reaction mixture was filtered and concentrated in vacuo to give the crude product. The residue was purified by prep-HPLC (column: Phenomenex luna C18 150 mm*40 mm*15 μm;mobile phase: [water(FA)-ACN]; B %: 50%-80%, 10 min). The compound tert-butyl 4-[2-[[1-[2-(6-methyl-7-oxo-1H-pyrrolo[2,3-c]pyridin-4-yl)-4-nitro-phenyl]-2-oxo-4-pyridyl]oxy]ethoxy]piperidine-1-carboxylate (150 mg, 233 μmol, 15% yield, 94% purity) was obtained as a colorless oil.

Step 6: Preparation of tert-butyl 4-[2-[[1-[4-amino-2-(6-methyl-7-oxo-1H-pyrrolo[2,3-c]pyridin-4-yl)phenyl]-2-oxo-4-pyridyl]oxy]ethoxy]piperidine-1-carboxylate. A mixture of tert-butyl 4-[2-[[1-[2-(6-methyl-7-oxo-1H-pyrrolo[2,3-c]pyridin-4-yl)-4-nitro-phenyl]-2-oxo-4-pyridyl]oxy]ethoxy]piperidine-1-carboxylate (100 mg, 165 μmol, 1.0 equiv), NH₄Cl (44.2 mg, 826 mol, 5.0 equiv), Fe (46.1 mg, 826 mol, 5.0 equiv) in EtOH (δ mL) and H₂O (3 mL) was degassed and purged with N₂ three times, and then the mixture was stirred at 70° C. for 12 h under N₂ atmosphere. The reaction mixture was filtered and concentrated in vacuo to give the crude product. The residue was purified by preparative TLC on silica gel (petroleum ether: EtOAc=0:1). Compound tert-butyl 4-[2-[[1-[4-amino-2-(6-methyl-7-oxo-1H-pyrrolo[2,3-c]pyridin-4-yl)phenyl]-2-oxo-4-pyridyl]oxy]ethoxy]piperidine-1-carboxylate (50.0 mg, 86.9 mol, 53% yield) was obtained as a colorless oil.

Step 7: Preparation of tert-butyl 4-[2-[[1-[4-(ethylsulfonylamino)-2-(6-methyl-7-oxo-1H-pyrrolo[2,3-c]pyridin-4-yl)phenyl]-2-oxo-4-pyridyl]oxy]ethoxy]piperidine-1-carboxylate. To a solution of tert-butyl 4-[2-[[1-[4-amino-2-(6-methyl-7-oxo-1H-pyrrolo[2,3-c]pyridin-4-yl)phenyl]-2-oxo-4-pyridyl]oxy]ethoxy]piperidine-1-carboxylate (50.0 mg, 86.9 μmol, 1.0 equiv) in DCM (3 mL) was added Et₃N (176 mg, 1.74 mmol, 242 μL, 20.0 equiv) and methanesulfonyl chloride (11.2 mg, 86.9 μmol, 8.21 μL, 1.0 equiv) at 0° C. The mixture was stirred at 25° C. for 12 h. The reaction mixture was concentrated in vacuo to give the crude product. The residue was purified by preparative TLC on silica gel (dichloromethane: petroleum ether=10:1). The compound tert-butyl 4-[2-[[1-[4-(ethylsulfonylamino)-2-(6-methyl-7-oxo-1H-pyrrolo[2,3-c]pyridin-4-yl)phenyl]-2-oxo-4-pyridyl]oxy]ethoxy]piperidine-1-carboxylate (44 mg, 66 mol, 76% yield) was obtained as a colorless oil.

Step 8: Preparation of N-[3-(6-methyl-7-oxo-1H-pyrrolo[2,3-c]pyridin-4-yl)-4-[2-oxo-4-[2-(4-piperidyloxy)ethoxy]-1-pyridyl]phenyl]ethanesulfonamide. To a solution of tert-butyl 4-[2-[[1-[4-(ethylsulfonylamino)-2-(6-methyl-7-oxo-¹H-pyrrolo[2,3-c]pyridin-4-yl)phenyl]-2-oxo-4-pyridyl]oxy]ethoxy]piperidine-1-carboxylate (50 mg, 75 4 mol, 1.0 equiv) in DCM (3 mL) was added TFA (4.62 g, 40.5 mmol, 3.00 mL, 541.0 equiv). The mixture was stirred at 25° C. for 1 h. The reaction mixture was concentrated in vacuo to give the crude product N-[3-(6-methyl-7-oxo-1H-pyrrolo[2,3-c]pyridin-4-yl)-4-[2-oxo-4-[2-(4-piperidyloxy) ethoxy]-1-pyridyl]phenyl]ethanesulfonamide (40 mg, 70. mol, 94% yield) as a white solid which was used in the next step without further purification.

Step 9: Preparation of N-[3-(3-chloro-4-cyano-phenoxy)-2,2,4,4-tetramethyl-cyclobutyl]-2-[4-[2-[[1-[4-(ethylsulfonylamino)-2-(6-methyl-7-oxo-1H-pyrrolo[2,3-c]pyridin-4-yl)phenyl]-2-oxo-4-pyridyl]oxy]ethoxy]-1-piperidyl]pyrimidine-5-carboxamide. To a solution of tert-butyl 4-[2-[[1-[4-(ethylsulfonylamino)-2-(6-methyl-7-oxo-1H-pyrrolo[2,3-c]pyridin-4-yl)phenyl]-2-oxo-4-pyridyl]oxy]ethoxy]piperidine-1-carboxylate (40 mg, 60 μmol, 1.0 equiv) in NMP (2 mL) was added K₂CO₃ (24.8 mg, 180 mol, 3.0 equiv) and 2-chloro-N-[3-(3-chloro-4-cyano-phenoxy)-2,2,4,4-tetramethyl-cyclobutyl]pyrimidine-5-carboxamide (25.1 mg, 59.9 μmol, 1.0 equiv). The mixture was stirred at 50° C. for 1 h. The reaction mixture was concentrated in vacuo to give the crude product. The residue was purified by prep-HPLC (column: Unisil 3-100 C18 Ultra 150 mm*50 mm*3 μm; mobile phase: [water(FA)-ACN]; B %: 52%-82%, 7 min). The compound N-[3-(3-chloro-4-cyano-phenoxy)-2,2,4,4-tetramethyl-cyclobutyl]-2-[4-[2-[[1-[4-(ethylsulfonylamino)-2-(6-methyl-7-oxo-1H-pyrrolo[2,3-c]pyridin-4-yl)phenyl]-2-oxo-4-pyridyl]oxy]ethoxy]-1-piperidyl]pyrimidine-5-carboxamide (18 mg, 18 μmol, 30% yield, 95% purity) was obtained as a white solid. ¹H NMR (CD₃OD, 400 MHz): δ 8.75 (s, 2H), 7.74 (d, J=8.8 Hz, 1H), 7.52-7.37 (m, 3H), 7.32-7.24 (m, 2H), 7.15 (d, J=2.4 Hz, 1H), 7.06-6.97 (m, 2H), 6.37-6.19 (m, 1H), 6.02-5.96 (m, 1H), 5.92 (d, J=2.4 Hz, 1H), 4.35-4.26 (m, 3H), 4.18-4.09 (m, 3H), 3.85 (m, 2H), 3.77-3.69 (m, 1H), 3.67-3.60 (m, 2H), 3.58 (s, 3H), 3.24 (s, 2H), 2.00-1.89 (m, 2H), 1.65-1.51 (m, 2H), 1.42-1.34 (m, 3H), 1.30 (s, 6H), 1.23 (s, 6H). LC-MS: MS (ES⁺): RT=2.655 min, m/z=950.2 [M+H⁺]; LCMS method: 25.

### Example 32—Synthesis of N-[3-(3-chloro-4-cyano-phenoxy)-2,2,4,4-tetramethyl-cyclobutyl]-2-[4-[1-[3-[4-isopropyl-2-(6-methyl-7-oxo-1H-pyrrolo[2,3-c]pyridin-4-yl)phenoxy]phenyl]azetidin-3-yl]piperazin-1-yl]pyrimidine-5-carboxamide (I-122)

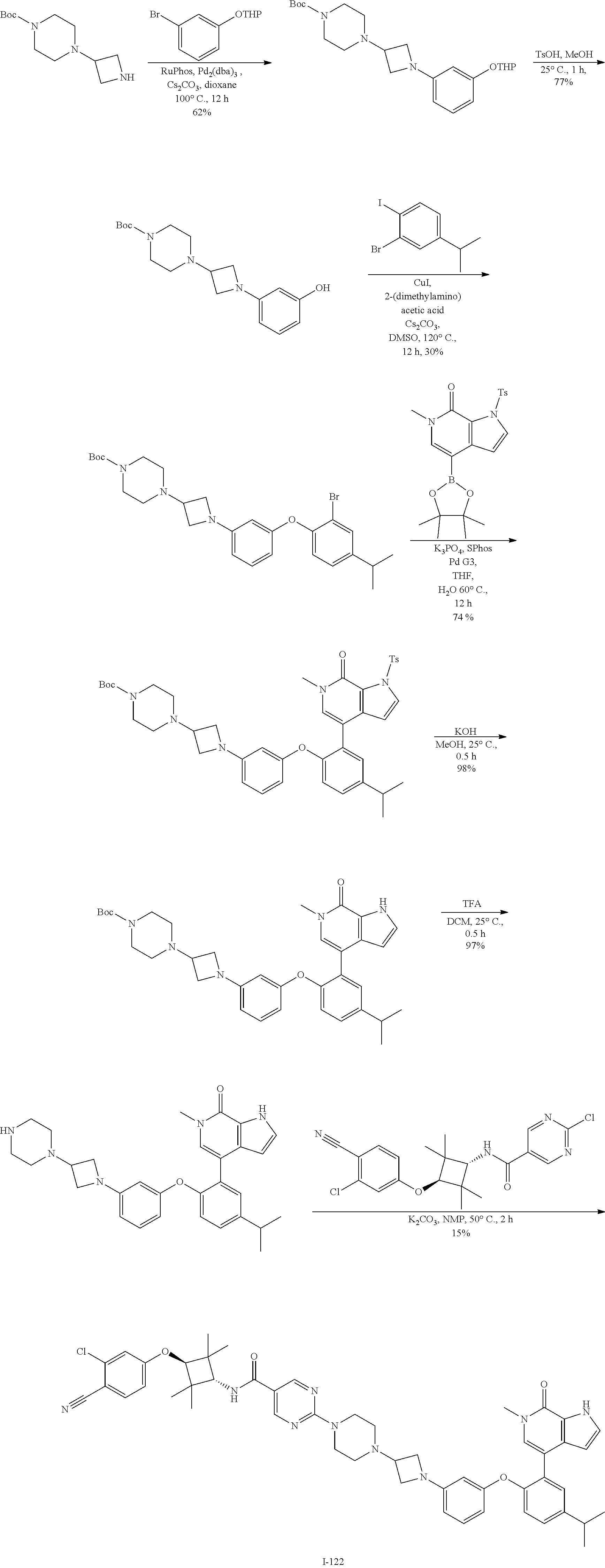

Step 1: Preparation of tert-butyl 4-[1-(3-tetrahydropyran-2-yloxyphenyl) azetidin-3-yl]piperazine-1-carboxylate. To a solution of tert-butyl 4-(azetidin-3-yl)piperazine-1-carboxylate (6.0 g, 25 mmol, 1.0 equiv) in dioxane (60 mL) and 2-(3-bromophenoxy)tetrahydropyran (9.5 g, 37 mmol, 1.5 equiv) was added RuPhos (1.1 g, 2.4 mmol, 0.1 equiv), Pd₂(dba)₃ (1.1 g, 1.2 mmol, 0.1 equiv) and Cs₂CO₃ (24.3 g, 74.5 mmol, 3.0 equiv). The mixture was stirred at 100° C. for 12 h. The mixture was filtered and concentrated. The residue was purified by column chromatography (SiO₂, petroleum ether: ethyl acetate=10:1 to dichloromethane: methanol=10:1) to give tert-butyl 4-[1-(3-tetrahydropyran-2-yloxyphenyl)azetidin-3-yl]piperazine-1-carboxylate (6.5 g, 62% yield) as a yellow solid.

Step 2: Preparation of tert-butyl 4-[1-(3-hydroxyphenyl)azetidin-3-yl]piperazine-1-carboxylate. To a solution of tert-butyl 4-[1-(3-tetrahydropyran-2-yloxyphenyl)azetidin-3-yl]piperazine-1-carboxylate (6.5 g, 15.5 mmol, 1.0 equiv) in MeOH (60 mL) was added TsOH (8.0 g, 46.7 mmol, 3.0 equiv). The mixture was stirred at 25° C. for 1 h. The mixture was quenched with 10 mL H₂O and extracted with EtOAc (3×10 mL). The combined organic layer was concentrated. The residue was purified by prep-TLC (SiO₂, dichloromethane: methanol=10:1) to give tert-butyl 4-[1-(3-hydroxyphenyl)azetidin-3-yl]piperazine-1-carboxylate (4.0 g, 77% yield) as a yellow solid.

Step 3: Preparation of tert-butyl 4-[1-[3-(2-bromo-4-isopropyl-phenoxy) phenyl]azetidin-3-yl]piperazine-1-carboxylate. A mixture of tert-butyl 4-[1-(3-hydroxyphenyl)azetidin-3-yl]piperazine-1-carboxylate (250 mg, 749 μmol, 1.0 equiv), 2-bromo-1-10 do-4-isopropyl-benzene (609 mg, 1.8 mmol, 2.5 equiv), CuI (114 mg, 599 μmol, 0.8 equiv), 2-(dimethylamino)acetic acid (61 mg, 599 μmol, 0.8 equiv) and Cs₂CO₃ (732 mg, 2.2 mmol, 3.0 equiv) in DMSO (9 mL) was stirred at 120° C. for 12 h under N₂ atmosphere. The mixture was diluted with water (20 mL) and extracted with EtOAc (3×10 mL). The combined organic layers were washed with brine (20 mL), dried over Na₂SO₄, filtered, and concentrated. The residue was purified by prep-TLC ((SiO₂, petroleum ether: ethyl acetate=3:1) to give tert-butyl 4-[1-[3-(2-bromo-4-isopropyl-phenoxy)phenyl]azetidin-3-yl]piperazine-1-carboxylate (120 mg, 30% yield).

Step 4: Preparation of tert-butyl 4-[1-[3-[4-isopropyl-2-[6-methyl-7-oxo-1-(p-tolylsulfonyl)pyrrolo[2,3-c]pyridin-4-yl]phenoxy]phenyl]azetidin-3-yl]piperazine-1-carboxylate. To a solution of tert-butyl 4-[1-[3-(2-bromo-4-isopropyl-phenoxy)phenyl]azetidin-3-yl]piperazine-1-carboxylate (140 mg, 263 mol, 1.0 equiv) and 6-methyl-1-(p-tolylsulfonyl)-4-(4,4,5,5-tetramethyl-1,3,2-dioxaborolan-2-yl)pyrrolo[2,3-c]pyridin-7-one (135 mg, 316 μmol, 1.2 equiv) in THF (7 mL) and H₂O (1.4 mL) was added K₃PO₄ (168 mg, 791 μmol, 3.0 equiv) and SPhos Pd G3 (20 mg, 26 μmol, 0.1 equiv). The mixture was stirred at 60° C. for 12 h. The mixture was filtered and the filtrate was concentrated. The residue was purified by column chromatography (SiO₂, petroleum ether: ethyl acetate=10:1 to 1:1) to give tert-butyl 4-[1-[3-[4-isopropyl-2-[6-methyl-7-oxo-1-(p-tolylsulfonyl)pyrrolo[2,3-c]pyridin-4-yl]phenoxy]phenyl]azetidin-3-yl]piperazine-1-carboxylate (147 mg, 74% yield).

Step 5: Preparation of tert-butyl 4-[1-[3-[4-isopropyl-2-(6-methyl-7-oxo-1H-pyrrolo[2,3-c]pyridin-4-yl)phenoxy]phenyl]azetidin-3-yl]piperazine-1-carboxylate. To a solution of tert-butyl 4-[1-[3-[4-isopropyl-2-[6-methyl-7-oxo-1-(p-tolylsulfonyl)pyrrolo[2,3-c]pyridin-4-yl]phenoxy]phenyl]azetidin-3-yl]piperazine-1-carboxylate (140 mg, 186 mol, 1.0 equiv) in MeOH (1 mL) was added KOH (188 mg, 3.3 mmol, 18.0 equiv). The mixture was stirred at 25° C. for 0.5 h. The mixture was diluted with water (20 mL) and extracted with DCM (3×10 mL). The combined organic layers were washed with brine (20 mL), dried over Na₂SO₄, filtered and concentrated. The residue was used in the next step without further purification.

Step 6: Preparation of 4-[5-isopropyl-2-[3-(3-piperazin-1-ylazetidin-1-yl)phenoxy]phenyl]-6-methyl-1H-pyrrolo[2,3-c]pyridin-7-one. To a solution of tert-butyl 4-[1-[3-[4-isopropyl-2-(6-methyl-7-oxo-1H-pyrrolo[2,3-c]pyridin-4-yl)phenoxy]phenyl]azetidin-3-yl]piperazine-1-carboxylate (55 mg, 92 mol, 1.0 equiv) in DCM (1 mL) was added TFA (0.5 mL). The mixture was stirred at 25° C. for 0.5 h and concentrated. The residue was used in the next step without further purification.

Step 7: Preparation of N-[3-(3-chloro-4-cyano-phenoxy)-2,2,4,4-tetramethyl-cyclobutyl]-2-[4-[1-[3-[4-isopropyl-2-(6-methyl-7-oxo-1H-pyrrolo[2,3-c]pyridin-4-yl)phenoxy]phenyl]azetidin-3-yl]piperazin-1-yl]pyrimidine-5-carboxamide. To a solution of 4-[5-isopropyl-2-[3-(3-piperazin-1-ylazetidin-1-yl)phenoxy]phenyl]-6-methyl-¹H-pyrrolo[2,3-c]pyridin-7-one (55 mg, 89 μmol, 1 equiv, TFA salt) in NMP (0.5 mL) was added K₂CO₃ (37 mg, 269 mol, 3.0 equiv) and 2-chloro-N-[3-(3-chloro-4-cyano-phenoxy)-2,2,4,4-tetramethyl-cyclobutyl]pyrimidine-5-carboxamide (37 mg, 89 μmol, 1.0 equiv). The mixture was stirred at 50° C. for 2 h. The reaction mixture was filtered and concentrated. The residue was purified by prep-HPLC(column: Phenomenex luna C18 150 mm*25 mm*m;mobile phase: [water(FA)-ACN];B %: 51%-81%, 10 min) to give N-[3-(3-chloro-4-cyano-phenoxy)-2,2,4,4-tetramethyl-cyclobutyl]-2-[4-[1-[3-[4-isopropyl-2-(6-methyl-7-oxo-1H-pyrrolo[2,3-c]pyridin-4-yl)phenoxy]phenyl]azetidin-3-yl]piperazin-1-yl]pyrimidine-5-carboxamide (12 mg, 15% yield).¹H NMR (400 MHz, CDCl₃) δ 10.14-9.88 (m, 1H), 8.72 (s, 2H), 7.58 (d, J=8.8 Hz, 1H), 7.37 (d, J=2.0 Hz, 1H), 7.24-7.21 (m, 1H), 7.20-7.14 (m, 1H), 7.09-6.93 (m, 4H), 6.88-6.76 (m, 1H), 6.45-6.36 (m, 1H), 6.29-6.20 (m, 1H), 6.16-6.07 (m, 1H), 6.03-5.89 (m, 2H), 4.14 (d, J=8.2 Hz, 1H), 4.05 (s, 1H), 4.03-3.94 (m, 4H), 3.93-3.87 (m, 2H), 3.71-3.64 (m, 2H), 3.61 (s, 3H), 3.40-3.30 (m, 1H), 3.04-2.88 (m, 1H), 2.58-2.42 (m, 4H), 1.30 (d, J=6.8 Hz, 6H), 1.24 (d, J=13.6 Hz, 12H).LC-MS: MS (ES⁺): RT=0.698 min, m/z=514.3 [M+H]⁺; LCMS Method: 25

### Example 33—Synthesis of N-[3-(3-chloro-4-cyano-phenoxy)-2,2,4,4-tetramethyl-cyclobutyl]-2-[4-[3-[3-[4-isopropyl-2-(6-methyl-7-oxo-1H-pyrrolo[2,3-c]pyridin-4-yl)phenoxy]phenyl]propyl]piperazin-1-yl]pyrimidine-5-carboxamide (I-125)

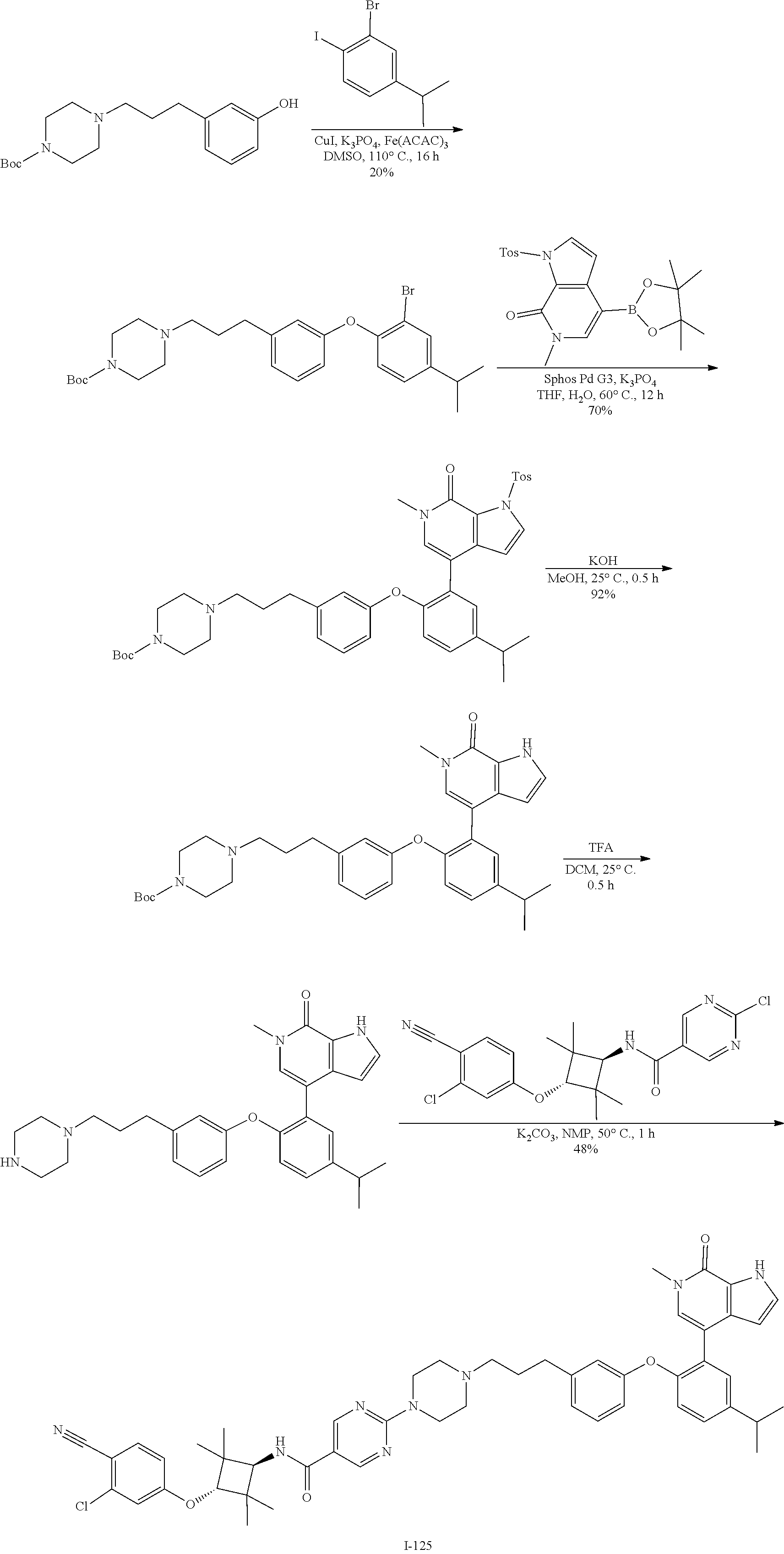

Step 1: Preparation of tert-butyl 4-[3-[3-(2-bromo-4-isopropyl-phenoxy) phenyl]propyl]piperazine-1-carboxylate. To a solution of tert-butyl 4-[3-(3-hydroxyphenyl)propyl]piperazine-1-carboxylate (400 mg, 1.25 mmol, 1.2 equiv) and 2-bromo-1-10 do-4-isopropyl-benzene (338 mg, 1.04 mmol, 1.0 equiv) in DMSO (4 mL) was added CuI (59 mg, 312 μmol, 0.3 equiv) and K₃PO₄ (662 mg, 3.12 mmol, 3.0 equiv), and Fe(acac)₃ (110 mg, 312 mol, 0.3 equiv). The mixture was stirred at 1100 C. for 16 h. To the reaction mixture was added 15 mL water and the mixture was extracted with EtOAc (15 mL×3). The combined organic phase was washed with brine (3×15 mL), dried over anhydrous Na₂SO₄, filtered and concentrated in vacuum. The residue was purified by column chromatography (SiO₂, ethyl acetate: methanol=1:0 to 15:1) to give tert-butyl 4-[3-[3-(2-bromo-4-isopropyl-phenoxy)phenyl]propyl]piperazine-1-carboxylate (110 mg, 20% yield) as a colorless oil.

Step 2: Preparation of tert-butyl 4-[3-[3-[4-isopropyl-2-[6-methyl-7-oxo-1-(p-tolylsulfonyl)pyrrolo[2,3-c]pyridin-4-yl]phenoxy]phenyl]propyl]piperazine-1-carboxylate. To a solution of tert-butyl 4-[3-[3-(2-bromo-4-isopropyl-phenoxy)phenyl]propyl]piperazine-1-carboxylate (110 mg, 212 mol, 1 equiv) and 6-methyl-1-(p-tolylsulfonyl)-4-(4,4,5,5-tetramethyl-1,3,2-dioxaborolan-2-yl)pyrrolo[2,3-c]pyridin-7-one (109 mg, 255 μmol, 1.2 equiv) in THF (5 mL) and H₂O(1 mL) was added SPhos Pd G₃ (16 mg, 21 μmol, 0.1 equiv) and K₃PO₄ (135 mg, 637 μmol, 3.0 equiv). The mixture was stirred at 60° C. for 12 h. To the reaction mixture was added 20 mL water and the mixture was extracted with EtOAc (15 mL×3). The combined organic phase was washed with brine (3×15 mL), dried over anhydrous Na₂SO₄, filtered and concentrated in vacuum. The residue was purified by column chromatography (SiO₂, petroleum ether: ethyl acetate=10:1 to 0:1) to give tert-butyl 4-[3-[3-[4-isopropyl-2-[6-methyl-7-oxo-1-(p-tolylsulfonyl)pyrrolo[2,3-c]pyridin-4-yl]phenoxy]phenyl]propyl]piperazine-1-carboxylate (110 mg, 70% yield) as a white solid.

Step 3: Preparation of tert-butyl 4-[3-[3-[4-isopropyl-2-(6-methyl-7-oxo-1H-pyrrolo[2,3-c]pyridin-4-yl)phenoxy]phenyl]propyl]piperazine-1-carboxylate. To a solution of tert-butyl 4-[3-[3-[4-isopropyl-2-[6-methyl-7-oxo-1-(p-tolylsulfonyl)pyrrolo[2,3-c]pyridin-4-yl]phenoxy]phenyl]propyl]piperazine-1-carboxylate (110 mg, 0.15 mmol, 1.0 equiv) in MeOH (3 mL) was added KOH (10 mg, 0.15 mmol, 1.0 equiv). The mixture was stirred at 25° C. for 1 h and concentrated. The residue was purified by prep-TLC (SiO₂, DCM: MeOH=10:1) to give tert-butyl 4-[3-[3-[4-isopropyl-2-(6-methyl-7-oxo-1H-pyrrolo[2,3-c]pyridin-4-yl)phenoxy]phenyl]propyl]piperazine-1-carboxylate (80 mg, 92% yield) as a colorless oil.

Step 4: Preparation of 4-[5-isopropyl-2-[3-(3-piperazin-1-ylpropyl)phenoxy]phenyl]-6-methyl-1H-pyrrolo[2,3-c]pyridin-7-one. To a solution of tert-butyl 4-[3-[3-[4-isopropyl-2-(6-methyl-7-oxo-1H-pyrrolo[2,3-c]pyridin-4-yl)phenoxy]phenyl]propyl]piperazine-1-carboxylate (80 mg, 0.14 mmol, 1.0 equiv) in DCM (3 mL) was added TFA (1 mL). The mixture was stirred at 25° C. for 0.5 h. Without purification, it was used for the next step directly.

Step 5: Preparation of N-[3-(3-chloro-4-cyano-phenoxy)-2,2,4,4-tetramethyl-cyclobutyl]-2-[4-[3-[3-[4-isopropyl-2-(6-methyl-7-oxo-1H-pyrrolo[2,3-c]pyridin-4-yl) phenoxy]phenyl]propyl]piperazin-1-yl]pyrimidine-5-carboxamide. To a solution of 4-[5-isopropyl-2-[3-(3-piperazin-1-ylpropyl)phenoxy]phenyl]-6-methyl-1H-pyrrolo[2,3-c]pyridin-7-one (40 mg, 66 μmol, 1.0 equiv, TFA salt) and 2-chloro-N-[3-(3-chloro-4-cyano-phenoxy)-2,2,4,4-tetramethyl-cyclobutyl]pyrimidine-5-carboxamide (28 mg, 66 μmol, 1.0 equiv) in NMP (2 mL) was added K₂CO₃ (28 mg, mol, 3 equiv). The mixture was stirred at 50° C. for 1 h and concentrated. The residue was purified by prep-HPLC (column: Unisil 3-100 C18 Ultra 150 mm*50 mm* 3 μm; mobile phase: [water(FA)-ACN];B %: 39%-69%, 7 min) to give N-[3-(3-chloro-4-cyano-phenoxy)-2,2,4,4-tetramethyl-cyclobutyl]-2-[4-[3-[3-[4-isopropyl-2-(6-methyl-7-oxo-1H-pyrrolo[2,3-c]pyridin-4-yl)phenoxy]phenyl]propyl]piperazin-1-yl]pyrimidine-5-carboxamide (28 mg, 47% yield, 99% purity) as a white solid. ¹H NMR (400 MHz, DMSO-d₆): δ 11.99 (s, 1H), 8.80 (s, 2H), 7.90 (d, J=8.76 Hz, 1H), 7.76 (d, J=7.24 Hz, 1H), 7.35 (d, J=2.24 Hz, 1H), 7.23 (d, J=2.24 Hz, 2H), 7.20-7.22 (m, 2H), 7.15-7.19 (m, 1H), 7.01 (d, J=8.84, 2.44 Hz, 1H), 6.93 (d, J=8.24 Hz, 1H), 6.85-6.90 (m, 1H), 6.64-6.75 (m, 2H), 6.19-6.23 (m, 1H), 4.29 (s, 1H), 4.04 (d, J=9.12 Hz, 1H), 3.66-3.88 (m, 2H), 3.50 (s, 3H), 3.32 (s, 9H), 2.92-2.97 (m, 1H), 2.60-2.74 (m, 1H), 2.30-2.36 (m, 1H), 1.66-1.76 (m, 1H), 1.25 (d, J=6.48 Hz, 6H), 1.21 (s, 6H), 1.11 (s, 6H). LC-MS: MS (ES⁺): RT=2.683 min, m/z=867.3 [M+H⁺]; LCMS method: 25

### Example 34—Synthesis of N-[3-(3-chloro-4-cyano-phenoxy)-2,2,4,4-tetramethyl-cyclobutyl]-2-[4-[2-[2-fluoro-3-[2-(6-methyl-7-oxo-1H-pyrrolo[2,3-c]pyridin-4-yl)-4-(trifluoromethylsulfonylamino)phenoxy]phenoxy]ethoxy]-1-piperidyl]pyrimidine-5-carboxamide (I-163)

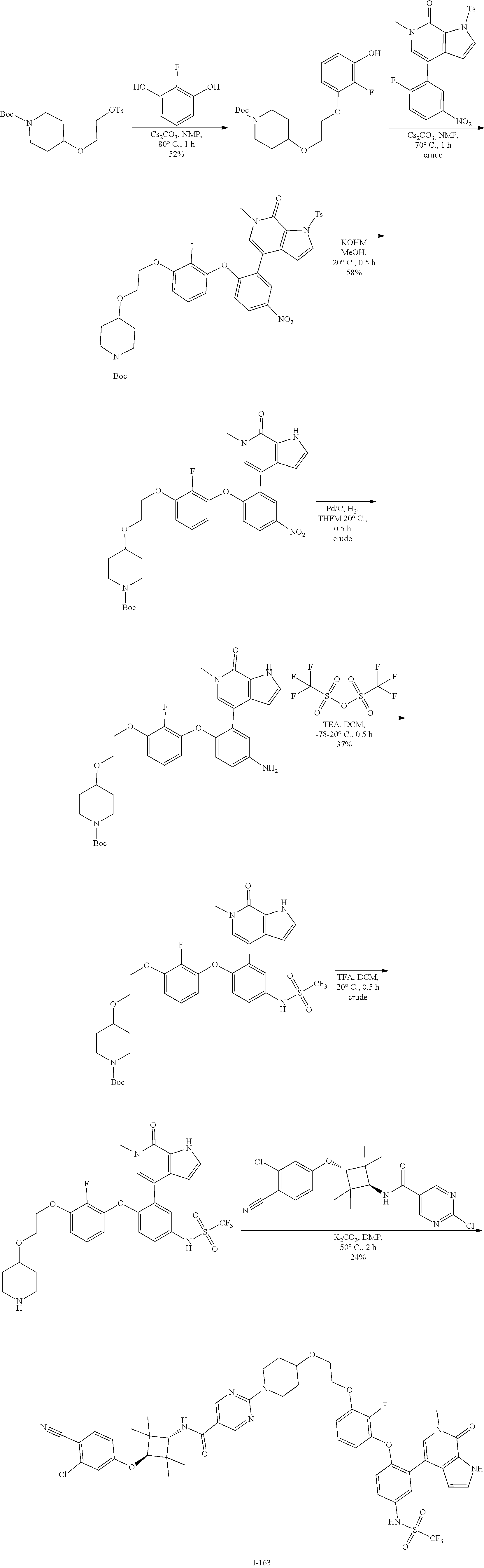

Step 1: Preparation of tert-butyl 4-[2-(2-fluoro-3-hydroxy-phenoxy)ethoxy]piperidine-1-carboxylate. To a solution of tert-butyl 4-[2-(p-tolylsulfonyloxy)ethoxy]piperidine-1-carboxylate (5 g, 12.52 mmol, 1 equiv) in NMP (30 mL) was added Cs₂CO₃ (12.23 g, 37.55 mmol, 3 equiv) and 2-fluorobenzene-1,3-diol (4.81 g, 37.55 mmol, 3 equiv). The mixture was stirred at 80° C. for 1 h and concentrated. The residue was purified by prep-HPLC (column: Kromasil Eternity XT 250 mm*80 mm*10 μm; mobile phase: [water( NH₄HCO₃)-ACN]; B %: 35%-65%, 20 min) to give tert-butyl 4-[2-(2-fluoro-3-hydroxy-phenoxy)ethoxy]piperidine-1-carboxylate (2.3 g, 52% yield) as a yellow oil.

Step 2: Preparation of tert-butyl 4-[2-[2-fluoro-3-[2-[6-methyl-7-oxo-1-(p-tolylsulfonyl)pyrrolo[2,3-c]pyridin-4-yl]-4-nitro-phenoxy]phenoxy]ethoxy]piperidine-1-carboxylate. To a solution of tert-butyl 4-[2-(2-fluoro-3-hydroxy-phenoxy)ethoxy]piperidine-1-carboxylate (966 mg, 2.72 mmol, 1 equiv) in NMP (10 mL) was added Cs₂CO₃ (2.66 g, 8.16 mmol, 3 eq) and 4-(2-fluoro-5-nitro-phenyl)-6-methyl-1-(p-tolylsulfonyl) pyrrolo[2,3-c]pyridin-7-one (1.2 g, 2.72 mmol, 1 equiv). The mixture was stirred at 70° C. for 1 h and concentrated to give crude product tert-butyl 4-[2-[2-fluoro-3-[2-[6-methyl-7-oxo-1-(p-tolylsulfonyl)pyrrolo[2,3-c]pyridin-4-yl]-4-nitro-phenoxy]phenoxy]ethoxy]piperidine-1-carboxylate (2.1 g).

Step 3: Preparation of tert-butyl 4-[2-[2-fluoro-3-[2-(6-methyl-7-oxo-1H-pyrrolo[2,3-c]pyridin-4-yl)-4-nitro-phenoxy]phenoxy]ethoxy]piperidine-1-carboxylate. To a solution of tert-butyl 4-[2-[2-fluoro-3-[2-[6-methyl-7-oxo-1-(p-tolylsulfonyl)pyrrolo[2,3-c]pyridin-4-yl]-4-nitro-phenoxy]phenoxy]ethoxy]piperidine-1-carboxylate (2.1 g, 2.70 mmol, 1 equiv) in MeOH (10 mL) was added KOH (455 mg, 8.11 mmol, 3 equiv). The mixture was stirred at 20° C. for 0.5 h and concentrated. The residue was purified by prep-HPLC (column: Waters Xbridge C18 150 mm*50 mm*10 um; mobile phase: [water(NH₄HCO₃)-ACN]; B %: 44%-74%, 10 min) to give tert-butyl 4-[2-[2-fluoro-3-[2-(6-methyl-7-oxo-1H-pyrrolo[2,3-c]pyridin-4-yl)-4-nitro-phenoxy]phenoxy]ethoxy]piperidine-1-carboxylate (970 mg, 58%).

Step 4: Preparation of tert-butyl 4-[2-[3-[4-amino-2-(6-methyl-7-oxo-1H-pyrrolo[2,3-c]pyridin-4-yl)phenoxy]-2-fluoro-phenoxy]ethoxy]piperidine-1-carboxylate. To a solution of tert-butyl 4-[2-[2-fluoro-3-[2-(6-methyl-7-oxo-1H-pyrrolo[2,3-c]pyridin-4-yl)-4-nitro-phenoxy]phenoxy]ethoxy]piperidine-1-carboxylate (970 mg, 1.56 mmol, 1 equiv) in THF (10 mL) was added Pd/C (1 g, 10% purity) under N₂ atmosphere. The suspension was degassed and purged with H₂ three times. The mixture was stirred under H₂ (50 psi) at 20° C. for 0.5 h and concentrated to give crude product tert-butyl 4-[2-[3-[4-amino-2-(6-methyl-7-oxo-1H-pyrrolo[2,3-c]pyridin-4-yl)phenoxy]-2-fluoro-phenoxy]ethoxy]piperidine-1-carboxylate (850 mg).

Step 5: Preparation of tert-butyl 4-[2-[2-fluoro-3-[2-(6-methyl-7-oxo-1H-pyrrolo[2,3-c]pyridin-4-yl)-4-(trifluoromethylsulfonylamino)phenoxy]phenoxy]ethoxy]piperidine-1-carboxylate. To a solution of tert-butyl 4-[2-[3-[4-amino-2-(6-methyl-7-oxo-1H-pyrrolo[2,3-c]pyridin-4-yl)phenoxy]-2-fluoro-phenoxy]ethoxy]piperidine-1-carboxylate (110 mg, 186 μmol, 1 equiv) in DCM (5 mL) was added TEA (376 mg, 3.71 mmol, 20 equiv) and Tf₂O (262 mg, 928 μmol, 5 equiv) at −78° C. The mixture was stirred at 20° C. for 0.5 h and concentrated. The residue was purified by prep-TLC (SiO₂, DCM: MeOH=10:1) to give tert-butyl 4-[2-[2-fluoro-3-[2-(6-methyl-7-oxo-1H-pyrrolo[2,3-c]pyridin-4-yl)-4-(trifluoro-methylsulfonylamino)phenoxy]phenoxy]ethoxy]piperidine-1-carboxylate (50 mg, 37% yield).

Step 6: Preparation of 1,1,1-trifluoro-N-[4-[2-fluoro-3-[2-(4-piperidyloxy) ethoxy]phenoxy]-3-(6-methyl-7-oxo-1H-pyrrolo[2,3-c]pyridin-4-yl)phenyl]methanesulfonamide. To a solution of tert-butyl 4-[2-[2-fluoro-3-[2-(6-methyl-7-oxo-1H-pyrrolo[2,3-c]pyridin-4-yl)-4-(trifluoromethylsulfonylamino)phenoxy]phenoxy]ethoxy]piperidine-1-carboxylate (40 mg, 55 μmol, 1 equiv) in DCM (1 mL) was added TFA (2 mL). The mixture was stirred at 20° C. for 0.2 h and concentrated to give crude product 1,1,1-trifluoro-N-[4-[2-fluoro-3-[2-(4-piperidyloxy)ethoxy]phenoxy]-3-(6-methyl-7-oxo-1H-pyrrolo[2,3-c]pyridin-4-yl)phenyl]methanesulfonamide (40 mg, TFA salt).

Step 7: Preparation of N-[3-(3-chloro-4-cyano-phenoxy)-2,2,4,4-tetramethyl-cyclobutyl]-2-[4-[2-[2-fluoro-3-[2-(6-methyl-7-oxo-1H-pyrrolo[2,3-c]pyridin-4-yl)-4-(trifluoromethylsulfonylamino)phenoxy]phenoxy]ethoxy]-1-piperidyl]pyrimidine-5-carboxamide. To a solution of 1,1,1-trifluoro-N-[4-[2-fluoro-3-[2-(4-piperidyloxy)ethoxy]phenoxy]-3-(6-methyl-7-oxo-1H-pyrrolo[2,3-c]pyridin-4-yl)phenyl]methanesulfonamide (20 mg, 27 μmol, 1 equiv, TFA salt) in NMP (1 mL) was added K₂CO₃ (11 mg, 81 μmol, 3 equiv) and 2-chloro-N-[3-(3-chloro-4-cyano-phenoxy)-2,2,4,4-tetramethyl-cyclobutyl]pyrimidine-5-carboxamide (11 mg, 27 μmol, 1 equiv). The mixture was stirred at 50° C. for 2 h and concentrated. The residue was purified by prep-HPLC (column: Phenomenex luna C18 150 mm*25 mm*10 μm; mobile phase: [water(FA)-ACN]; B %: 50%-80%, 10 min) to give N-[3-(3-chloro-4-cyano-phenoxy)-2,2,4,4-tetramethyl-cyclobutyl]-2-[4-[2-[2-fluoro-3-[2-(6-methyl-7-oxo-1H-pyrrolo[2,3-c]pyridin-4-yl)-4-(trifluoromethylsulfonylamino) phenoxy]phenoxy]ethoxy]-1-piperidyl]pyrimidine-5-carboxamide (δ mg, 21% yield). LC-MS: MS (ES⁺): RT=2.880 min, m/z=1007.2 [M+H⁺]; LCMS method: 40.

### Example 35—Synthesis of N-[3-(3-chloro-4-cyano-phenoxy)-2,2,4,4-tetramethyl-cyclobutyl]-2-[4-[2-[2-cyclopropyl-2-[4-ethylsulfonyl-2-(6-methyl-7-oxo-1H-pyrrolo[2,3-c]pyridin-4-yl)phenoxy]ethoxy]ethoxy]-1-piperidyl]pyrimidine-5-carboxamide (I-186)

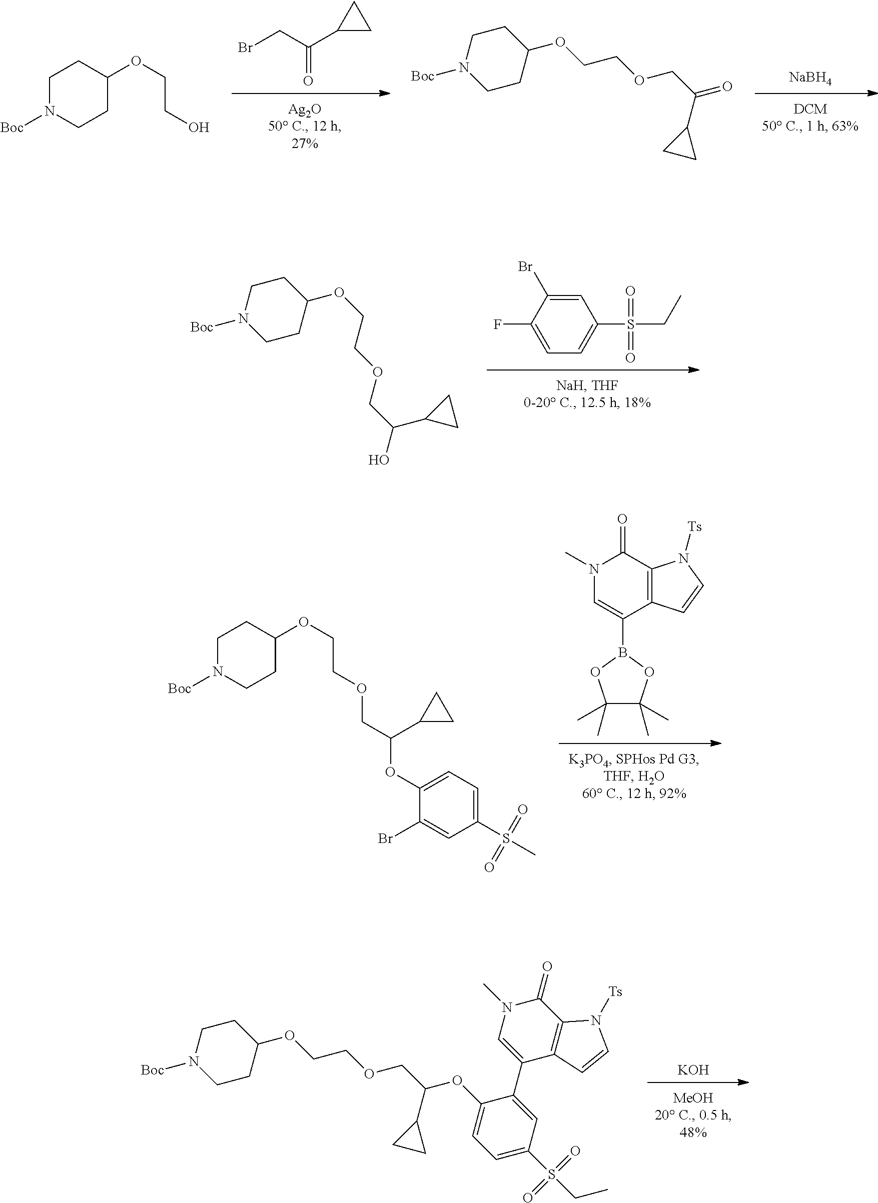

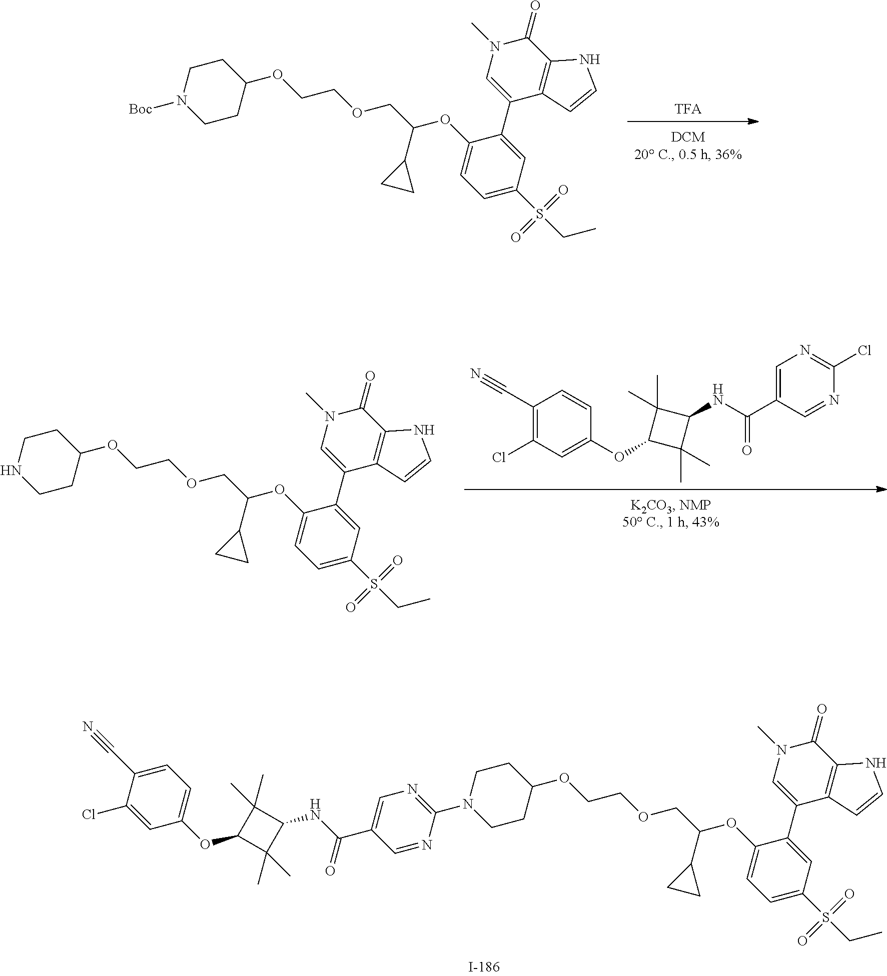 [SMILES: CCS(=O)(=O)c1ccc(OC(COCCOC2CCN(C(=O)OC(C)(C)C)CC2)C2CC2)c(-c2cn(C)c(=O)c3[nH]ccc23)c1 ; CCS(=O)(=O)c1ccc(OC(COCCOC2CCNCC2)C2CC2)c(-c2cn(C)c(=O)c3[nH]ccc23)c1 ; CC1(C)[C@H](NC(=O)c2cnc(Cl)nc2)C(C)(C)[C@H]1Oc1ccc(C#N)c(Cl)c1 ; "TFA" ; CCS(=O)(=O)c1ccc(OC(COCCOC2CCN(c3ncc(C(=O)N[C@H]4C(C)(C)[C@H](Oc5ccc(C#N)c(Cl)c5)C4(C)C)cn3)CC2)C2CC2)c(-c2cn(C)c(=O)c3[nH]ccc23)c1]

Step 1: Preparation of tert-butyl 4-[2-(2-cyclopropyl-2-oxo-ethoxy)ethoxy]piperidine-1-carboxylate. To a solution of 2-bromo-1-cyclopropyl-ethanone (2.2 g, 13 mmol, 2 equiv) and tert-butyl 4-(2-hydroxyethoxy)piperidine-1-carboxylate (1.66 g, 7 mmol, 1 equiv) was added Ag₂O (1.88 g, 8 mmol, 1.2 equiv). The mixture was stirred at 50° C. for 12 h and concentrated. The residue was purified by prep-HPLC (FA condition;column: Shim-pack C18 150 mm*25 mm* 10 μm;mobile phase: [water(FA)-ACN];B %: 35%-65%, 10 min) to give tert-butyl 4-[2-(2-cyclopropyl-2-oxo-ethoxy)ethoxy]piperidine-1-carboxylate (600 mg, 27% yield).

Step 2: Preparation of tert-butyl 4-[2-(2-cyclopropyl-2-hydroxy-ethoxy)ethoxy]piperidine-1-carboxylate. To a solution of tert-butyl 4-[2-(2-cyclopropyl-2-oxo-ethoxy) ethoxy]piperidine-1-carboxylate (600 mg, 2 mmol, 1 equiv) in DCM (9 mL) was added NaBH₄ (173 mg, 5 mmol, 2.5 equiv). The mixture was stirred at 50° C. for 1 h. The mixture was extracted with EA (3×20 mL). The combined organic layers were washed with brine, dried over Na₂SO₄, filtered and concentrated. The residue was purified by column chromatography (SiO₂, petroleum ether: ethyl acetate=1:1 to 1:2) to give tert-butyl 4-[2-(2-cyclopropyl-2-hydroxy-ethoxy)ethoxy]piperidine-1-carboxylate (380 mg, 63% yield).

Step 3: Preparation of tert-butyl 4-[2-[2-(2-bromo-4-ethylsulfonyl- phenoxy)-2-cyclopropyl-ethoxy]ethoxy]piperidine-1-carboxylate. To a solution of tert-butyl 4-[2-(2-cyclopropyl-2-hydroxy-ethoxy)ethoxy]piperidine-1-carboxylate (500 mg, 1.52 mmol, 1 equiv) in THF (5 mL) was added NaH (122 mg, 3 mmol, 60% purity, 2 equiv) at 0° C., after 0.5 h, 2-bromo-4-ethylsulfonyl-1-fluoro-benzene (446 mg, 1.67 mmol, 1.1 equiv) was added. The mixture was stirred at 20° C. for 12 h. The reaction mixture was and concentrated. The residue was purified by prep-HPLC (FA condition; column: Phenomenex luna C18 150 mm*40 mm*15 μm; mobile phase: [water (FA)-ACN]; B %: 63%-93%, 10 min) to give tert-butyl 4-[2-[2-(2-bromo-4-ethylsulfonyl- phenoxy)-2-cyclopropyl-ethoxy]ethoxy]piperidine-1-carboxylate (154 mg, 18% yield).

Step 4: Preparation of tert-butyl 4-[2-[2-cyclopropyl-2-[4-ethylsulfonyl-2-[6-methyl-7-oxo-1-(p-tolylsulfonyl)pyrrolo[2,3-c]pyridine-4-yl]phenoxy]ethoxy]ethoxy]piperidine-1-carboxylate. To a solution of tert-butyl 4-[2-[2-(2-bromo-4-ethylsulfonyl-phenoxy)-2-cyclopropyl-ethoxy]ethoxy]piperidine-1-carboxylate (154 mg, 267 4 mol, 1 equiv) and 6-methyl-1-(p-tolylsulfonyl)-4-(4,4,5,5-tetramethyl-1,3,2-dioxaborolan-2-yl)pyrrolo[2,3-c]pyridin-7-one (137 mg, 320 mol, 1.2 equiv) in THF (2 mL) and H₂O (0.4 mL) was added K₃PO₄ (170 mg, 801 μmol, 3 equiv) and SPhos Pd G3 (21 mg, 27 μmol, 0.1 equiv). The mixture was stirred at 60° C. for 12 h. The mixture was concentrated and the residue was purified by prep-TLC (SiO₂, petroleum ether: ethyl acetate=0:1) to give tert-butyl 4-[2-[2-cyclopropyl-2-[4-ethylsulfonyl-2-[6-methyl-7-oxo-1-(p-tolylsulfonyl) pyrrolo[2,3-c]pyridine-4-yl]phenoxy]ethoxy]ethoxy]piperidine-1-carboxylate (195 mg, 92% yield).

Step 5: Preparation of tert-butyl 4-[2-[2-cyclopropyl-2-[4-ethylsulfonyl-2-(6-methyl-7-oxo-1H-pyrrolo[2,3-c]pyridin-4-yl) phenoxy]ethoxy]ethoxy]piperidine-1-carboxylate. To a solution of tert-butyl 4-[2-[2-cyclopropyl-2-[4-ethylsulfonyl-2-[6-methyl-7-oxo-1-(p-tolylsulfonyl) pyrrolo[2,3-c]pyridin-4-yl]phenoxy]ethoxy]ethoxy]piperidine-1-carboxylate (195 mg, 244 mol, 1 equiv) in MeOH (2 mL) was added KOH (165 mg, 3 mmol, 12 equiv). The mixture was stirred at 20° C. for 0.5 h and quenched with 50 mL water. The organic phase was separated, dried over anhydrous sodium sulfate, filtered and concentrated. The residue was purified by prep-TLC (SiO₂, DCM: MeOH=10:1) to give tert-butyl 4-[2-[2-cyclopropyl-2-[4-ethylsulfonyl-2-(6-methyl-7-oxo-1H-pyrrolo[2,3-c]pyridin-4-yl) phenoxy]ethoxy]ethoxy]piperidine-1-carboxylate (76 mg, 48% yield).

Step 6: Preparation of 4-[2-[1-cyclopropyl-2-[2-(4-piperidyloxy)ethoxy]ethoxy]-5-ethylsulfonyl-phenyl]-6-methyl-1H-pyrrolo[2,3-c]pyridin-7-one. To a solution of tert-butyl 4-[2-[2-cyclopropyl-2-[4-ethylsulfonyl-2-(6-methyl-7-oxo-1H-pyrrolo [2,3-c]pyridin-4-yl)phenoxy]ethoxy]ethoxy]piperidine-1-carboxylate (66 mg, 103 μmol, 1 equiv) in DCM (1 mL) was added TFA (0.5 mL). The mixture was stirred at 20° C. for 0.5 h and concentrated. The residue was purified by prep-HPLC (neutral condition; column: Waters Xbridge 150 mm*25 mm*5 μm; mobile phase: [water(NH₄HCO₃)-ACN]; B %: 24%-54%, 8 min) to give 4-[2-[1-cyclopropyl-2-[2-(4-piperidyloxy)ethoxy]ethoxy]-5-ethylsulfonyl-phenyl]-6-methyl-1H-pyrrolo[2,3-c]pyridin-7-one (20 mg, 36% yield).

Step 7: Preparation of N-[3-(3-chloro-4-cyano-phenoxy)-2,2,4,4-tetramethyl-cyclobutyl]-2-[4-[2-[2-cyclopropyl-2-[4-ethylsulfonyl-2-(6-methyl-7-oxo-1H-pyrrolo[2,3-c]pyridin-4-yl)phenoxy]ethoxy]ethoxy]-1-piperidyl]pyrimidine-5-carboxamide. To a solution of 4-[2-[1-cyclopropyl-2-[2-(4-piperidyloxy)ethoxy]ethoxy]-5-ethylsulfonyl-phenyl]-6-methyl-1H-pyrrolo[2,3-c]pyridin-7-one (18 mg, 33 μmol, 1 equiv) in NMP (1 mL) was added K₂CO₃ (23 mg, 166 mol, 5 equiv) and 2-chloro-N-[3-(3-chloro-4-cyano-phenoxy)-2,2,4,4-tetramethyl-cyclobutyl]pyrimidine-5-carboxamide (15 mg, 37 μmol, 1.1 equiv). The mixture was stirred at 50° C. for 1 h. The residue was purified by prep-HPLC (FA condition; column: Unisil 3-100 C18 Ultra 150 mm*50 mm*3 μm; mobile phase: [water(FA)-ACN]; B %: 61%-91%, 7 min) to give N-[3-(3-chloro-4-cyano-phenoxy)-2,2,4,4-tetramethyl-cyclobutyl]-2-[4-[2-[2-cyclopropyl-2-[4-ethylsulfonyl-2-(6-methyl-7-oxo-1H-pyrrolo[2,3-c]pyridin-4-yl)phenoxy]ethoxy]ethoxy]-1-piperidyl]pyrimidine-5-carboxamide (14 mg, 43% yield). ¹H NMR (400 MHz, CDCl₃): δ 8.06 (d, J=2.4 Hz, 1H), 7.74 (m, 1H), 7.19 (d, J=8.8 Hz, 1H), 4.18 (m, 1H), 3.87-3.74 (m, 4H), 3.70-3.62 (m, 2H), 3.60-3.51 (m, 2H), 3.48-3.39 (m, 1H), 3.14-3.04 (m, 4H), 1.88-1.73 (m, 2H), 1.55-1.47 (m, 2H), 1.46 (s, 9H), 1.30-1.27 (m, 3H), 1.17-1.11 (m, 1H), 0.60-0.56 (m, 2H), 0.42-0.26 (m, 2H). LC-MS: MS (ES⁺): RT=3.075 min, m/z=926.2 [M+H⁺]; LCMS method: 25.

### Example 36—Synthesis of N-[3-(3-chloro-4-cyano-phenoxy)-2,2,4,4-tetramethyl-cyclobutyl]-2-[4-[[1-[2-fluoro-3-[2-(6-methyl-7-oxo-1H-pyrrolo[2,3-c]pyridin-4-yl)-4-methylsulfonyl-phenoxy]phenyl]azetidin-3-yl]methyl]-1-piperidyl]pyrimidine-5-carboxamide (I-189)

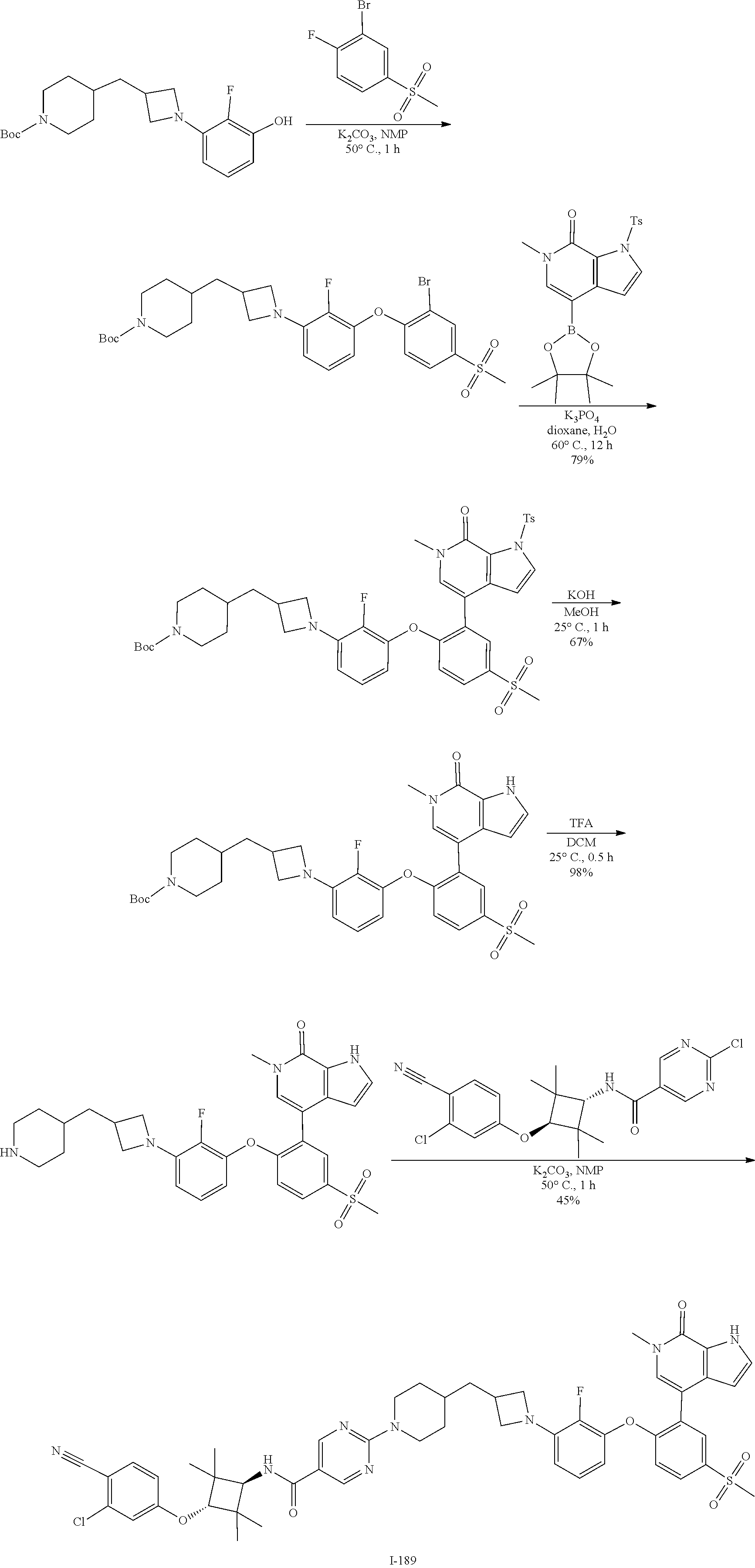

Step 1: Preparation of tert-butyl 4-[[1-[3-(2-bromo-4-methylsulfonyl-phenoxy)-2-fluoro-phenyl]azetidin-3-yl]methyl]piperidine-1-carboxylate. To a solution of tert-butyl 4-[[1-(2-fluoro-3-hydroxy-phenyl) azetidin-3-yl]methyl]piperidine-1-carboxylate (200 mg, 548 μmol, 1.0 equiv) and 2-bromo-1-fluoro-4-methylsulfonyl-benzene (138 mg, 548 μmol, 1.0 equiv) in NMP (4 mL) was added K₂CO₃ (447 mg, 1.37 mmol, 2.5 equiv). The mixture was stirred at 70° C. for 1 h. The reaction mixture was filtered and concentrated under reduced pressure to give a residue. The residue was purified by prep-TLC (SiO₂, DCM: MeOH=15:1) to give the compound tert-butyl 4-[[1-[3-(2-bromo-4-methylsulfonyl-phenoxy)-2-fluoro-phenyl]azetidin-3-yl]methyl]piperidine-1-carboxylate (250 mg, 76% yield).

Step 2: Preparation of tert-butyl 4-[[1-[2-fluoro-3-[2-[6-methyl-7-oxo-1-(p-tolylsulfonyl)pyrrolo[2,3-c]pyridin-4-yl]-4-methylsulfonyl-phenoxy]phenyl]azetidin-3-yl]methyl]piperidine-1-carboxylate. To a solution of tert-butyl 4-[[1-[3-(2-bromo-4-methylsulfonyl-phenoxy)-2-fluoro-phenyl]azetidin-3-yl]methyl]piperidine-1-carboxylate (230 mg, 384 μmol, 1.0 equiv) and 6-methyl-1-(p-tolylsulfonyl)-4-(4,4,5,5-tetramethyl-1,3,2-dioxaborolan-2-yl)pyrrolo[2,3-c]pyridin-7-one (181 mg, 423 mol, 1.1 equiv) in H₂O (1 mL) and THF (5 mL) was added K₃PO₄ (245 mg, 1.15 mmol, 3.0 equiv) and dicyclohexyl-[2-(2,6-dimethoxyphenyl)phenyl]phosphane;methanesulfonate;(2-phenylanilino)palladium(1+) (30 mg, 38 mol, 0.1 equiv). The mixture was stirred at 60° C. for 12 h. The reaction mixture was filtered and concentrated under reduced pressure to give a residue. The residue was purified by prep-TLC (SiO₂, DCM: MeOH=20:1) to give the compound tert-butyl 4-[[1-[2-fluoro-3-[2-[6-methyl-7-oxo-1-(p-tolylsulfonyl)pyrrolo[2,3-c]pyridin-4-yl]-4-methylsulfonyl-phenoxy]phenyl]azetidin-3-yl]methyl]piperidine-1-carboxylate (250 mg, 79% yield).

Step 3: Preparation of tert-butyl 4-[[1-[2-fluoro-3-[2-(6-methyl-7-oxo-1H-pyrrolo[2,3-c]pyridin-4-yl)-4-methylsulfonyl-phenoxy]phenyl]azetidin-3-yl]methyl]piperidine-1-carboxylate. To a solution of tert-butyl 4-[[1-[2-fluoro-3-[2-[6-methyl-7-oxo-1-(p-tolylsulfonyl)pyrrolo[2,3-c]pyridin-4-yl]-4-methylsulfonyl-phenoxy]phenyl]azetidin-3-yl]methyl]piperidine-1-carboxylate (100 mg, 122 mol, 1.0 equiv) in MeOH (2 mL) was added KOH (20 mg, 366 μmol, 3.0 equiv). The mixture was stirred at 25° C. for 1 h. The reaction mixture was filtered and concentrated under reduced pressure to give a residue. The residue was purified by prep-TLC (SiO₂, DCM: MeOH=15:1) to give the compound tert-butyl 4-[[1-[2-fluoro-3-[2-(6-methyl-7-oxo-1H-pyrrolo[2,3-c]pyridin-4-yl)-4-methylsulfonyl-phenoxy]phenyl]azetidin-3-yl]methyl]piperidine-1-carboxylate (55 mg, 67% yield) as a yellow oil.

Step 4: Preparation of 4-[2-[2-fluoro-3-[3-(4-piperidylmethyl) azetidin-1-yl]phenoxy]-5-methylsulfonyl-phenyl]-6-methyl-1H-pyrrolo [2, 3-c]pyridin-7-one. To a solution of tert-butyl 4-[[1-[2-fluoro-3-[2-(6-methyl-7-oxo-1H-pyrrolo[2,3-c]pyridin-4-yl)-4-methylsulfonyl-phenoxy]phenyl]azetidin-3-yl]methyl]piperidine-1-carboxylate (55 mg, 82 μmol, 1.0 equiv) in DCM (2 mL) was added TFA (0.5 mL). The mixture was stirred at 25° C. for 0.5 h. The reaction mixture was filtered and concentrated. The compound 4-[2-[2-fluoro-3-[3-(4-piperidylmethyl) azetidin-1-yl]phenoxy]-5-methylsulfonyl-phenyl]-6-methyl-1H-pyrrolo [2, 3-c]pyridin-7-one (46 mg, 98% yield) was obtained and used directly or the next step.

Step 5: Preparation of N-[3-(3-chloro-4-cyano-phenoxy)-2,2,4,4-tetramethyl-cyclobutyl]-2-[4-[[1-[2-fluoro-3-[2-(6-methyl-7-oxo-1H-pyrrolo[2,3-c]pyridin-4-yl)-4-methylsulfonyl-phenoxy]phenyl]azetidin-3-yl]methyl]-1-piperidyl]pyrimidine-5-carboxamide. To a solution of 4-[2-[2-fluoro-3-[3-(4-piperidylmethyl)azetidin-1-yl]phenoxy]-5-methylsulfonyl-phenyl]-6-methyl-¹H-pyrrolo[2,3-c]pyridin-7-one (46 mg, 81 μmol, 1.0 equiv) and 2-chloro-N-[3-(3-chloro-4-cyano-phenoxy)-2,2,4,4-tetramethyl-cyclobutyl]pyrimidine-5-carboxamide (34 mg, 81 mol, 1.0 equiv) in NMP (1 mL) was added K₂CO₃ (33 mg, 244 μmol, 3.0 equiv). The mixture was stirred at 50° C. for 1 h. The mixture was filtered and concentrated. The residue was purified by prep-HPLC (column: Unisil 3-100 C18 Ultra 150 mm*50 mm*3 μm; mobile phase: [water (FA)-ACN]; B %: 68%-98%, 7 min) to give the compound N-[3-(3-chloro-4-cyano-phenoxy)-2,2,4,4-tetramethyl-cyclobutyl]-2-[4-[[1-[2-fluoro-3-[2-(6-methyl-7-oxo-1H-pyrrolo[2,3-c]pyridin-4-yl)-4-methylsulfonyl-phenoxy]phenyl]azetidin-3-yl]methyl]-1-piperidyl]pyrimidine-5-carboxamide (35 mg, 45% yield). ¹H NMR (400 MHz, DMSO): δ 12.11 (s, 1H), 8.75 (s, 2H), 7.97 (d, J=2.4 Hz, 1H), 7.94-7.83 (m, 2H), 7.72 (d, J=9.3 Hz, 1H), 7.43 (s, 1H), 7.36-7.26 (m, 1H), 7.22 (d, J=2.3 Hz, 1H), 7.11-6.88 (m, 3H), 6.57 (t, J=6.9 Hz, 1H), 6.42 (t, J=8.1 Hz, 1H), 6.27 (d, J=2.8 Hz, 1H), 4.76-4.72 (m, 2H), 4.29 (s, 1H), 4.18-4.01 (m, 3H), 3.59 (s, 3H), 3.55 (t, J=6.2 Hz, 2H), 3.26 (s, 3H), 2.93 (t, J=11.7 Hz, 3H), 1.74-1.70 (m, 2H), 1.58 (d, J=6.0 Hz, 3H), 1.22 (s, 6H), 1.11 (s, 8H). LC-MS: MS (ES⁺): RT=3.364 min, m/z=947.2 [M+H⁺]; LCMS method: 25.

### Example 37—Synthesis of N-[3-(3-chloro-4-cyano-phenoxy)-2,2,4,4-tetramethyl-cyclobutyl]-2-[4-[3-[3-[2-(6-methyl-7-oxo-1H-pyrrolo[2,3-c]pyridin-4-yl)phenoxy]phenyl]propyl]piperazin-1-yl]pyrimidine-5-carboxamide (I-223)

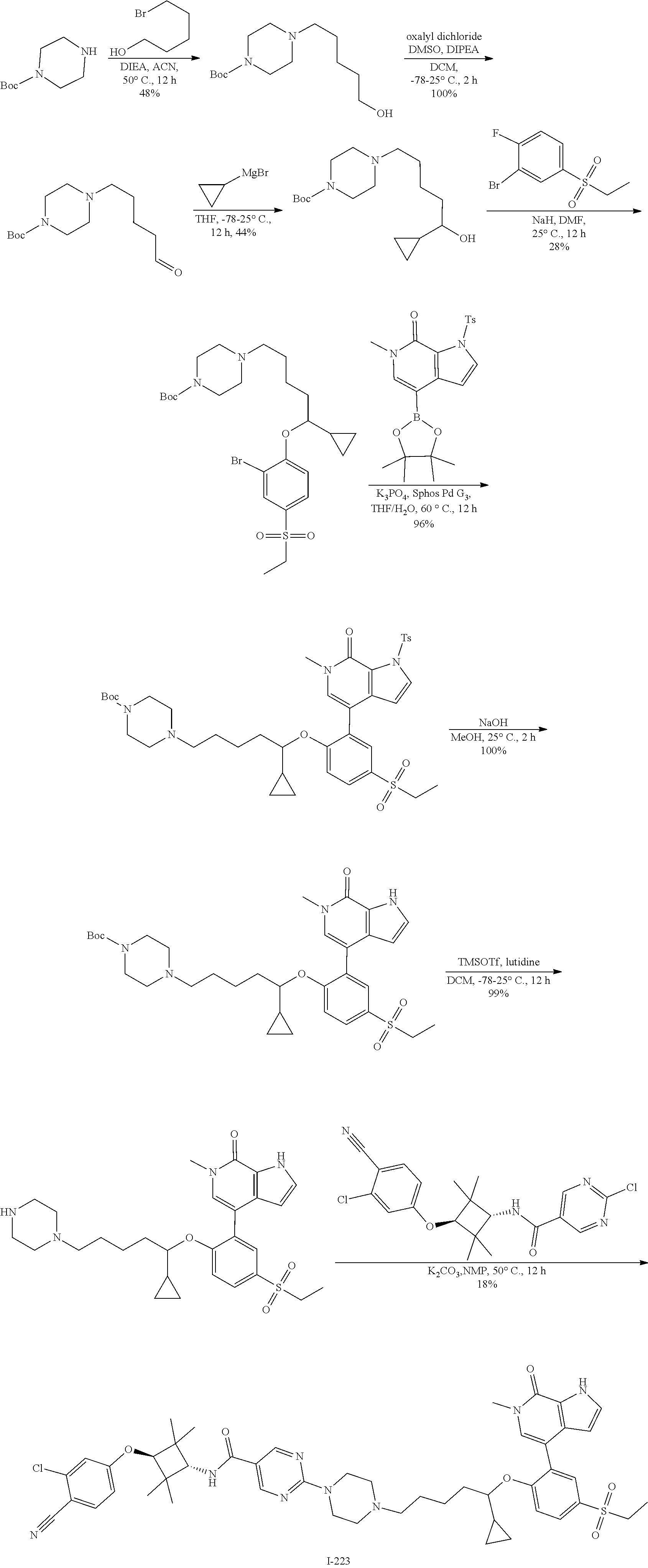

Step 1: Preparation of tert-butyl 4-(5-hydroxypentyl)piperazine-1-carboxylate. A mixture of tert-butyl piperazine-1-carboxylate (10.0 g, 53.7 mmol, 1.0 equiv), 5-bromopentan-1-ol (8.97 g, 53.7 mmol, 1.0 equiv), DIEA (20.8 g, 161 mmol, 28 mL, 3.0 equiv) in MeCN (100 mL) was degassed and purged with N₂ three times, and then the mixture was stirred at 50° C. for 12 h under an N₂ atmosphere. The reaction mixture was concentrated under reduced pressure to give a residue. The residue was purified by column chromatography (SiO₂, petroleum ether: ethyl acetate=1:1 to 0:1). The compound tert-butyl 4-(5-hydroxypentyl)piperazine-1-carboxylate (7.00 g, 25.7 mmol, 48% yield) was obtained as a yellow solid.

Step 2: Preparation of tert-butyl 4-(5-oxopentyl)piperazine-1-carboxylate. DMSO (1.50 g, 19.2 mmol, 1.5 mL, 5.2 equiv) was slowly added to a solution of oxalyl dichloride (1.45 g, 11.4 mmol, 1 mL, 3.1 equiv) in distilled DCM (15 mL) at −78° C. and the mixture was stirred at −78° C. for 0.5 h. Then tert-butyl 4-(5-hydroxypentyl)piperazine-1-carboxylate (1.00 g, 3.67 mmol, 1.0 equiv) was added and the mixture was stirred at −78° C. for 1 h. There after DIPEA (3.34 g, 25.8 mmol, 4.5 mL, 7.0 equiv) was slowly added at -78° C., and the mixture was stirred at 25° C. for 0.5 h. The crude product tert-butyl 4-(5-oxopentyl)piperazine-1-carboxylate (0.99 g, 3.66 mmol, 100% yield) was obtained as a colorless oil and was used in the next step without further purification.

Step 3: Preparation of tert-butyl 4-(5-cyclopropyl-5-hydroxy-pentyl)piperazine-1-carboxylate. To a solution of tert-butyl 4-(5-oxopentyl)piperazine-1-carboxylate (0.99 g, 3.66 nmol, 1.0 equiv) in DCM (15 mL) was added bromo(cyclopropyl) magnesium (0.5 M, 150 mL, 20.5 equiv) at −78° C. The mixture was stirred at −78° C.-25° C. for 12 h. The reaction mixture was quenched by addition water 50 mL at 25° C., and then diluted with DCM 50 mL and washed with brine 50 mL, dried over Na₂SO₄, filtered and concentrated under reduced pressure to give a residue. The residue was purified by prep-HPLC (FA condition; column: Shim-pack C18 150 mm*25 mm*10 um; mobile phase: [water(FA)-ACN]; B %: 2%-32%, 10 min). Compound tert-butyl 4-(5-cyclopropyl-5-hydroxy-pentyl)piperazine-1-carboxylate (0.50 g, 1.60 mmol, 44% yield) was obtained as a white solid.

Step 4: Preparation of tert-butyl 4-[5-(2-bromo-4-ethylsulfonyl-phenoxy)-5-cyclopropyl-pentyl]piperazine-1-carboxylate. To a mixture of NaH (100 mg, 2.50 mmol, 60% purity, 1.6 equiv) in DMF (5 mL) was added a solution of tert-butyl 4-(5-cyclopropyl-5-hydroxy-pentyl)piperazine-1-carboxylate (0.50 g, 1.60 mmol, 1.0 equiv) in DMF (2 mL) at 0° C. and stirred at 0° C. for 0.5 h. Then a solution of 2-bromo-4-ethylsulfonyl-1-fluoro-benzene (640 mg, 2.40 mmol, 1.5 equiv) in DMF (2 mL) was added at 0° C. and stirred at 25° C. for 12 h. The reaction mixture was poured into an ice/water mixture (50 mL) which was at 0° C., and then diluted with ethyl acetate 50 mL and the aqueous phase was extracted with ethyl acetate (50 mL×3). The combined organic layers were washed with brine 50 mL, dried over Na₂SO₄, filtered and concentrated under reduced pressure to give a residue. The residue was purified by prep-HPLC (FA condition; column: Unisil 3-100 C18 Ultra 150 mm*50 mm*3 μm; mobile phase: [water(FA)-ACN]; B %: 20%-50%, 7 min). The compound tert-butyl 4-[5-(2-bromo-4-ethylsulfonyl-phenoxy)-5-cyclopropyl-pentyl]piperazine-1-carboxylate (250 mg, 447 μmol, 28% yield) was obtained as a colorless gum.

Step 5: Preparation of tert-butyl 4-[5-cyclopropyl-5-[4-ethylsulfonyl-2-[6-methyl-7-oxo-1-(p-tolylsulfonyl)pyrrolo[2,3-c]pyridin-4-yl]phenoxy]pentyl]piperazine-1-carboxylate. A mixture of tert-butyl 4-[5-(2-bromo-4-ethylsulfonyl-phenoxy)-5-cyclopropyl-pentyl]piperazine-1-carboxylate (75.0 mg, 134 mol, 1.0 equiv), 6-methyl-1-(p-tolylsulfonyl)-4-(4,4,5,5-tetramethyl-1,3,2-dioxaborolan-2-yl)pyrrolo[2,3-c]pyridin-7-one (150 mg, 350 μmol, 2.6 equiv), K₃PO₄ (85.0 mg, 400 mol, 3.0 equiv) and SPhos Pd G₃ (25.0 mg, 32.0 μmol, 0.2 equiv) in THF (10 mL) and water (2 mL) was stirred at 60° C. for 12 h under an N₂ atmosphere. The reaction mixture was concentrated under reduced pressure to give a residue. The residue was purified by prep-TLC (SiO₂, DCM: MeOH=10:1). The compound tert-butyl 4-[5-cyclopropyl-5-[4-ethylsulfonyl-2-[6-methyl-7-oxo-1-(p-tolylsulfonyl)pyrrolo[2,3-c]pyridin-4-yl]phenoxy]pentyl]piperazine-1-carboxylate (100 mg, 128 μmol, 96% yield) was obtained as a white solid.

Step 6: Preparation of tert-butyl 4-[5-cyclopropyl-5-[4-ethylsulfonyl-2-(6-methyl-7-oxo-1H-pyrrolo[2,3-c]pyridin-4-yl)phenoxy]pentyl]piperazine-1-carboxylate. A mixture of tert-butyl 4-[5-cyclopropyl-5-[4-ethylsulfonyl-2-[6-methyl-7-oxo-1-(p-tolylsulfonyl)pyrrolo[2,3-c]pyridin-4-yl]phenoxy]pentyl]piperazine-1-carboxylate (100 mg, 128 μmol, 1.0 equiv) in MeOH (1 mL) was added NaOH (1 M, 1.92 mL, 15.0 equiv) and the mixture was stirred at 25° C. for 2 h under an N₂ atmosphere. The reaction mixture was poured into 30 mL water, then diluted with EtOAc (10 mL) and extracted with EtOAc (20 mL×3). The combined organic layers were washed with water (10 mL×3), dried over Na₂SO₄, filtered and concentrated under reduced pressure to give a residue. The crude product tert-butyl 4-[5-cyclopropyl-5-[4-ethylsulfonyl-2-(6-methyl-7-oxo-1H-pyrrolo[2,3-c]pyridin-4-yl)phenoxy]pentyl]piperazine-1-carboxylate (80.0 mg, 128 μmol, 100% yield) was obtained as a white gum was used in the next step without further purification.

Step 7: Preparation of 4-[2-(1-cyclopropyl-5-piperazin-1-yl-pentoxy)-5-ethylsulfonyl-phenyl]-6-methyl-1H-pyrrolo[2,3-c]pyridin-7-one. A mixture of tert-butyl 4-[5-cyclopropyl-5-[4-ethylsulfonyl-2-(6-methyl-7-oxo-1H-pyrrolo[2,3-c]pyridin-4-yl)phenoxy]pentyl]piperazine-1-carboxylate (70.0 mg, 112 4 mol, 1.0 equiv) and 2,6-dimethylpyridine (193 mg, 1.80 mmol, 210 μL, 16.1 equiv) in DCM (3 mL) was added trimethylsilyl trifluoromethanesulfonate (258 mg, 1.16 mmol, 210 μL, 10.4 equiv) at −78° C. and the mixture was stirred at -78-25° C. for 12 h under an N₂ atmosphere. The reaction mixture was concentrated under reduced pressure to give a residue. The crude product 4-[2-(1-cyclopropyl-5-piperazin-1-yl-pentoxy)-5-ethylsulfonyl-phenyl]-6-methyl-1H-pyrrolo[2,3-c]pyridin-7-one (58.0 mg, 110 mol, 99% yield) was obtained as a white gum was used in the next step without further purification.

Step 8: Preparation of N-[3-(3-chloro-4-cyano-phenoxy)-2,2,4,4-tetramethyl-cyclobutyl]-2-[4-[5-cyclopropyl-5-[4-ethylsulfonyl-2-(6-methyl-7-oxo-1H-pyrrolo[2,3-c]pyridin-4-yl)phenoxy]pentyl]piperazin-1-yl]pyrimidine-5-carboxamide. To A mixture of 4-[2-(1-cyclopropyl-5-piperazin-1-yl-pentoxy)-5-ethylsulfonyl-phenyl]-6-methyl-1H-pyrrolo[2,3-c]pyridin-7-one (50.0 mg, 94.9 μmol, 1.0 equiv) and 2-chloro-N-[3-(3-chloro-4-cyano-phenoxy)-2,2,4,4-tetramethyl-cyclobutyl]pyrimidine-5-carboxamide (40.0 mg, 95.4 μmol, 1.0 equiv) in NMP (0.5 mL) was added K₂CO₃ (40.00 mg, 289.42 μmol, 3.05 equiv) and the mixture was stirred at 50° C. for 12 h under an N₂ atmosphere. The reaction mixture was concentrated under reduced pressure to give a residue. The residue was purified by prep-HPLC (FA condition; column: Phenomenex Luna C18 150 mm*25 mm*10 μm; mobile phase: [water(FA)-ACN]; B %: 28%-58%, 9 min). The compound N-[3-(3-chloro-4-cyano-phenoxy)-2,2,4,4-tetramethyl-cyclobutyl]-2-[4-[5-cyclopropyl-5-[4-ethylsulfonyl-2-(6-methyl-7-oxo-1H-pyrrolo[2,3-c]pyridin-4-yl)phenoxy]pentyl]piperazin-1-yl]pyrimidine-5-carboxamide (16.1 mg, 16.8 μmol, 18% yield, 95% purity) was obtained as a yellow solid. Spectra: LC-MS: MS (ES⁺): RT=1.984 min, m/z=909.4 [M+H⁺], LCMS Method: 25. ¹H NMR: (400 MHz, METHANOL-d4) δ=8.77 (s, 2H), 8.44 (br s, 1H), 7.90-7.84 (m, 2H), 7.72 (d, J=8.8 Hz, 1H), 7.38-7.34 (m, 2H), 7.29 (s, 1H), 7.13 (d, J=2.4 Hz, 1H), 7.01-6.96 (m, 1H), 6.25 (d, J=2.8 Hz, 1H), 4.27 (s, 1H), 4.14 (s, 1H), 4.11-4.05 (m, 1H), 3.95 (br s, 4H), 3.71 (s, 3H), 3.27-3.19 (m, 2H), 2.67-2.59 (m, 4H), 2.47-2.33 (m, 2H), 1.74-1.67 (m, 2H), 1.53-1.43 (m, 2H), 1.31 (br s, 3H), 1.29-1.23 (m, 10H), 1.21 (s, 6H), 1.06-0.95 (m, 1H), 0.55-0.42 (m, 2H), 0.34-0.22 (m, 2H)

### Example 38-Synthesis of N-[3-(3-chloro-4-cyano-phenoxy)-2,2,4,4-tetramethyl-cyclobutyl]-2-[4-[4-cyclopropyl-5-[4-ethylsulfonyl-2-(6-methyl-7-oxo-1H-pyrrolo[2,3-c]pyridin-4-yl)phenoxy]pentyl]piperazin-1-yl]pyrimidine-5-carboxamide (I-231)

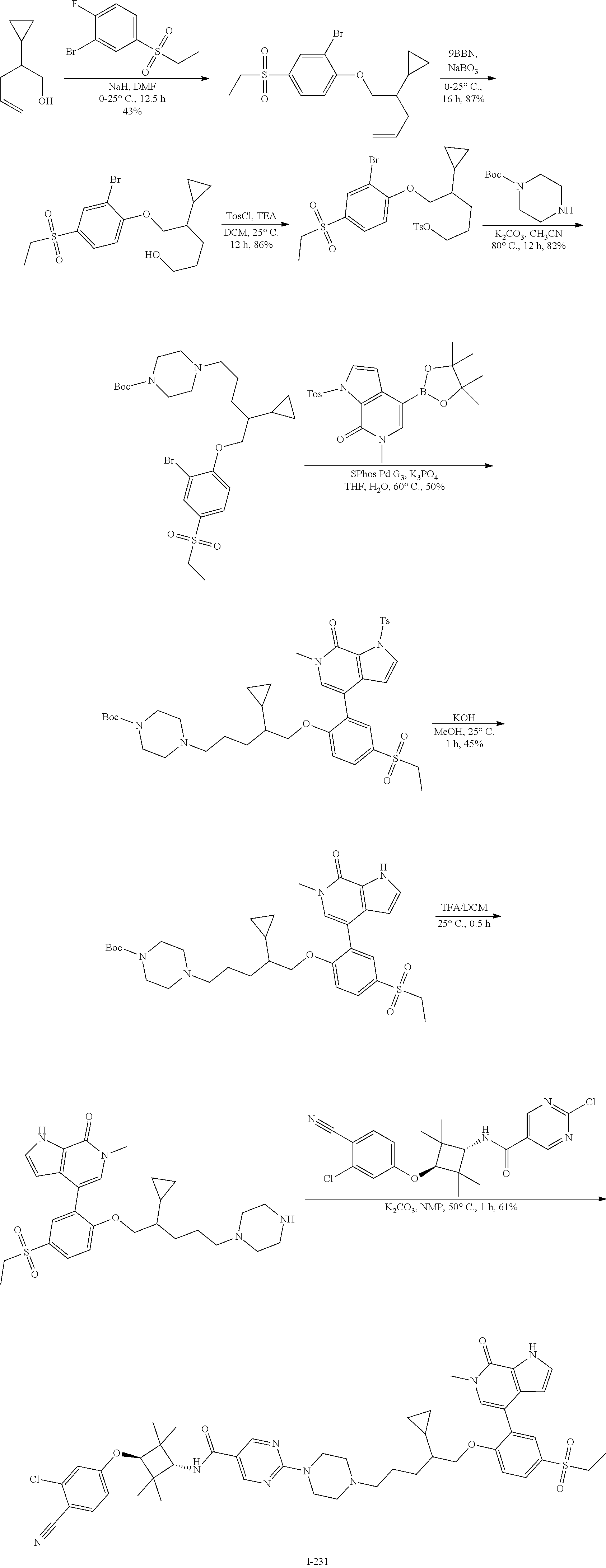

Step 1: Preparation of 2-bromo-1-(2-cyclopropylpent-4-enoxy)-4-ethylsulfonyl-benzene. To a mixture of NaH (451.6 mg, 11.2 mmol, 60% purity, 1.5 equiv) in THF (2 mL) was added a solution of 2-cyclopropylpent-4-en-1-ol (950 mg, 7.5 mmol, 1.0 equiv) in DMF (2 mL) at 0° C. and stirred at 0° C. for 0.5 h. Then a solution of 2-bromo-4-ethylsulfonyl-1-fluoro-benzene (3.0 g, 11.2 mmol, 1.5 equiv) in DMF (5 mL) was added at 0° C. and stirred at 25° C. for 12 h. The reaction mixture was acidified by HOAc to pH=5˜6. The residue was purified by prep-HPLC (column: Phenomenex luna C18 150 mm*40 mm*15 μm; mobile phase: [water (FA)-ACN]; B %: 58%-88%, 10 min) to give 2-bromo-1-(2-cyclopropylpent-4-enoxy)-4-ethylsulfonyl-benzene (1.2 g, 43% yield) as a yellow oil.

Step 2: Preparation of 5-(2-bromo-4-ethylsulfonyl-phenoxy)-4-cyclopropyl-pentan-1-ol. To 9-BBN (0.5 M, 19.3 mL, 3.0 equiv) in THF (12 mL) was added to 2-bromo-1-(2-cyclopropylpent-4-enoxy)-4-ethylsulfonyl-benzene (1.2 g, 3.2 mmol, 1.0 equiv) under an atmosphere of N₂ at 0° C. and the mixture was stirred at 25° C. for 4 h. Then sodium 3-oxidodioxaborirane tetrahydrate (1.98 g, 12.8 mmol, 2.5 mL, 4.0 equiv) and H₂O (8 mL) were added and the mixture was stirred at 25° C. for 12 h. The reaction mixture was concentrated to afford the crude product. The residue was purified by column chromatography on silica gel (petroleum ether: ethyl acetate=20:1 to 1:1) to give 5-(2-bromo-4-ethylsulfonyl-phenoxy)-4-cyclopropyl-pentan-1-ol (1.1 g, 87% yield) as a colorless oil.

Step 3: Preparation of [5-(2-bromo-4-ethylsulfonyl-phenoxy)-4-cyclopropyl-pentyl]4-methylbenzenesulfonate. To a solution of 5-(2-bromo-4-ethylsulfonyl-phenoxy)-4-cyclopropyl-pentan-1-ol (1.1 g, 2.8 mmol, 1.0 equiv) in DCM (10 mL) was added TosCl (1.0 g, 5.6 mmol, 2.0 equiv) and TEA (853.3 mg, 8.4 mmol, 1.1 mL, 3.0 equiv). The mixture was stirred at 25° C. for 12 h. To the reaction mixture was added water (50 mL) and the mixture was extracted with EtOAc (50 mL). The combined organic phase was washed with brine (50 mL×3), dried over anhydrous Na₂SO₄, filtered and concentrated in vacuum. The crude product was purified by column chromatography on silica gel (petroleum ether: ethyl acetate=20:1 to 2:1) to give [5-(2-bromo-4-ethylsulfonyl-phenoxy)-4-cyclopropyl-pentyl]4-methylbenzenesulfonate (1.3 g, 86% yield) as a yellow oil.

Step 4: Preparation of tert-butyl 4-[5-(2-bromo-4-ethylsulfonyl-phenoxy)-4-cyclopropyl-pentyl]piperazine-1-carboxylate. To a solution of [5-(2-bromo-4-ethyl-sulfonyl-phenoxy)-4-cyclopropyl-pentyl]4-methylbenzenesulfonate (1.3 g, 2.3 mmol, 1.0 equiv), tert-butyl piperazine-1-carboxylate; hydrochloride (531 mg, 2.30 mmol, 1.0 equiv) in MeCN (15 mL) was added K₂CO₃ (988 mg, 7.10 mmol, 3.0 equiv). The mixture was stirred at 80° C. for 12 h. The reaction mixture was concentrated to afford crude product. The residue was purified by column chromatography (SiO₂, petroleum ether: ethyl acetate=90:1 to 70:1) to give the compound tert-butyl 4-[5-(2-bromo-4-ethylsulfonyl-phenoxy)-4-cyclopropyl-pentyl]piperazine-1-carboxylate (1.1 g, 1.9 mmol, 82% yield) as a yellow oil.

Step 5: Preparation of tert-butyl 4-[4-cyclopropyl-5-[4-ethylsulfonyl-2-[6-methyl-7-oxo-1-(p-tolylsulfonyl)pyrrolo[2,3-c]pyridin-4-yl]phenoxy]pentyl]piperazine-1-carboxylate. A mixture of tert-butyl 4-[5-(2-bromo-4-ethylsulfonyl-phenoxy)-4-cyclopropyl-pentyl]piperazine-1-carboxylate (200 mg, 357 mol, 1.0 equiv), 6-methyl-1-(p-tolylsulfonyl)-4-(4,4,5,5-tetramethyl-1,3,2-dioxaborolan-2-yl)pyrrolo[2,3-c]pyridin-7-one (199 mg, 465 μmol, 1.3 equiv), K₃PO₄ (227.6 mg, 1.1 mmol, 3.0 equiv) and SPhos Pd G₃ (27.8 mg, 35.7 μmol, 0.1 equiv) in THF (2 mL) and H₂O (0.4 mL), and then the mixture was stirred at 60° C. for 3 h. The reaction mixture was concentrated to afford the crude product. The residue was purified by column chromatography (SiO₂, petroleum ether: ethyl acetate=3:1 to 0:1) to give compound tert-butyl 4-[4-cyclopropyl-5-[4-ethylsulfonyl-2-[6-methyl-7-oxo-1-(p-tolylsulfonyl)pyrrolo[2,3-c]pyridin-4-yl]phenoxy]pentyl]piperazine-1-carboxylate (0.14 g, 50% yield) as a yellow oil.

Step 6: Preparation of tert-butyl 4-[4-cyclopropyl-5-[4-ethylsulfonyl-2-(6-methyl-7-oxo-1H-pyrrolo[2,3-c]pyridin-4-yl)phenoxy]pentyl]piperazine-1-carboxylate. To a solution of tert-butyl 4-[4-cyclopropyl-5-[4-ethylsulfonyl-2-[6-methyl-7-oxo-1-(p-tolylsulfonyl)pyrrolo[2,3-c]pyridin-4-yl]phenoxy]pentyl]piperazine-1-carboxylate (140 mg, 179 μmol, 1.0 equiv) in MeOH (3 mL) was added KOH (1 M, 5 mL, 27.8 equiv). The mixture was stirred at 25° C. for 1 h. The residue was diluted with H₂O (20 mL) and extracted with EA (20 mL×3). The combined organic layers were dried over anhydrous Na₂SO₄, filtered and concentrated under reduced pressure to give a residue. The residue was purified by prep-HPLC (column: Waters Xbridge 150 mm*25 mm*5 μm; mobile phase: [water (NH₄HCO₃)-ACN]; B %: 47%-77%, 8 min) to give the compound tert-butyl 4-[4-cyclopropyl-5-[4-ethylsulfonyl-2-(6-methyl-7-oxo-1H-pyrrolo[2,3-c]pyridin-4-yl) phenoxy]pentyl]piperazine-1-carboxylate (0.05 g, 45% yield) as a yellow oil.

Step 7: Preparation of 4-[2-(2-cyclopropyl-5-piperazin-1-yl-pentoxy)-5-ethylsulfonyl-phenyl]-6-methyl-1H-pyrrolo [2, 3-c]pyridin-7-one. To a solution of tert-butyl 4-[4-cyclopropyl-5-[4-ethylsulfonyl-2-(6-methyl-7-oxo-1H-pyrrolo [2, 3-c]pyridin-4-yl)phenoxy]pentyl]piperazine-1-carboxylate (40 mg, 63.8 μmol, 1.0 equiv) in DCM (1 mL) was added TFA (0.5 mL). The mixture was stirred at 25° C. for 1 h. The reaction mixture was concentrated under reduced pressure to give the compound 4-[2-(2-cyclopropyl-5-piperazin-1-yl-pentoxy)-5-ethylsulfonyl-phenyl]-6-methyl-1H-pyrrolo [2, 3-c]pyridin-7-one (40 mg, 98% yield, TFA salt) as a colorless oil.

Step 8: Preparation of N-[3-(3-chloro-4-cyano-phenoxy)-2,2,4,4-tetramethyl-cyclobutyl]-2-[4-[4-cyclopropyl-5-[4-ethylsulfonyl-2-(6-methyl-7-oxo-1H-pyrrolo[2,3-c]pyridin-4-yl)phenoxy]pentyl]piperazin-1-yl]pyrimidine-5-carboxamide. To a solution of 4-[2-(2-cyclopropyl-5-piperazin-1-yl-pentoxy)-5-ethylsulfonyl-phenyl]-6-methyl-1H-pyrrolo[2,3-c]pyridin-7-one (40 mg, 62.4 μmol, 1.0 equiv, TFA salt), 2-chloro-N-[3-(3-chloro-4-cyano-phenoxy)-2,2,4,4-tetramethyl-cyclobutyl]pyrimidine-5-carboxamide (28.8 mg, 68.6 μmol, 1.1 equiv) in NMP (1 mL) was added K₂CO₃ (25.8 mg, 187.2 μmol, 3.0 equiv). The mixture was stirred at 50° C. for 1 h. The reaction mixture was filtered and concentrated under reduced pressure to give a residue. The residue was purified by prep-HPLC (column: Waters Xbridge 150 mm*25 mm*5 um; mobile phase: [water(NH₄HCO₃)-ACN]; B %: 60%-90%, 8 min) to give the compound N-[3-(3-chloro-4-cyano-phenoxy)-2,2,4,4-tetramethyl-cyclobutyl]-2-[4-[4-cyclopropyl-5-[4-ethylsulfonyl-2-(6-methyl-7-oxo-1H-pyrrolo[2,3-c]pyridin-4-yl)phenoxy]pentyl]piperazin-1-yl]pyrimidine-5-carboxamide (35 mg, 61% yield) as a white solid. LC-MS: MS (ES⁺): RT=2.42 min, m/z=910.3 [M+H⁺]; LCMS method: 25. ¹H NMR (400 MHz, CD₃OD): δ 8.75 (s, 2H), 7.90-7.97 (m, 1H), 7.86-7.90 (m, 1H), 7.73 (d, J=8.80 Hz, 1H), 7.31-7.44 (m, 2H), 7.29 (s, 1H), 7.09-7.17 (m, 1H), 6.94-7.02 (m, 1H), 6.19 (d, J=2.81 Hz, 1H), 4.27 (s, 1H), 4.05-4.22 (m, 3H), 3.82-3.92 (m, 4H), 3.70 (s, 3H), 3.23 (d, J=7.46 Hz, 2H), 2.30-2.47 (m, 4H), 2.09-2.19 (m, 2H), 1.13-1.55 (m, 19H), 0.85-1.05 (m, 1H), 0.60 (s, 2H), 0.30-0.40 (m, 1H), 0.00-0.17 (m, 2H).

### Example 39—Synthesis of N-3-(3-chloro-4-cyanophenoxy)-2,2,4,4-tetramethyl-cyclobutyl)-2-(4-(4-cyclopropyl-5-(4-(2-hydroxypropan-2-yl)-2-(6-methyl-7-oxo-6,7-dihydro-1H-pyrrolo[2,3-c]pyridin-4-yl)phenoxy)pentyl)piperazin-1-yl)pyrimidine-5-carboxamide (I-237)

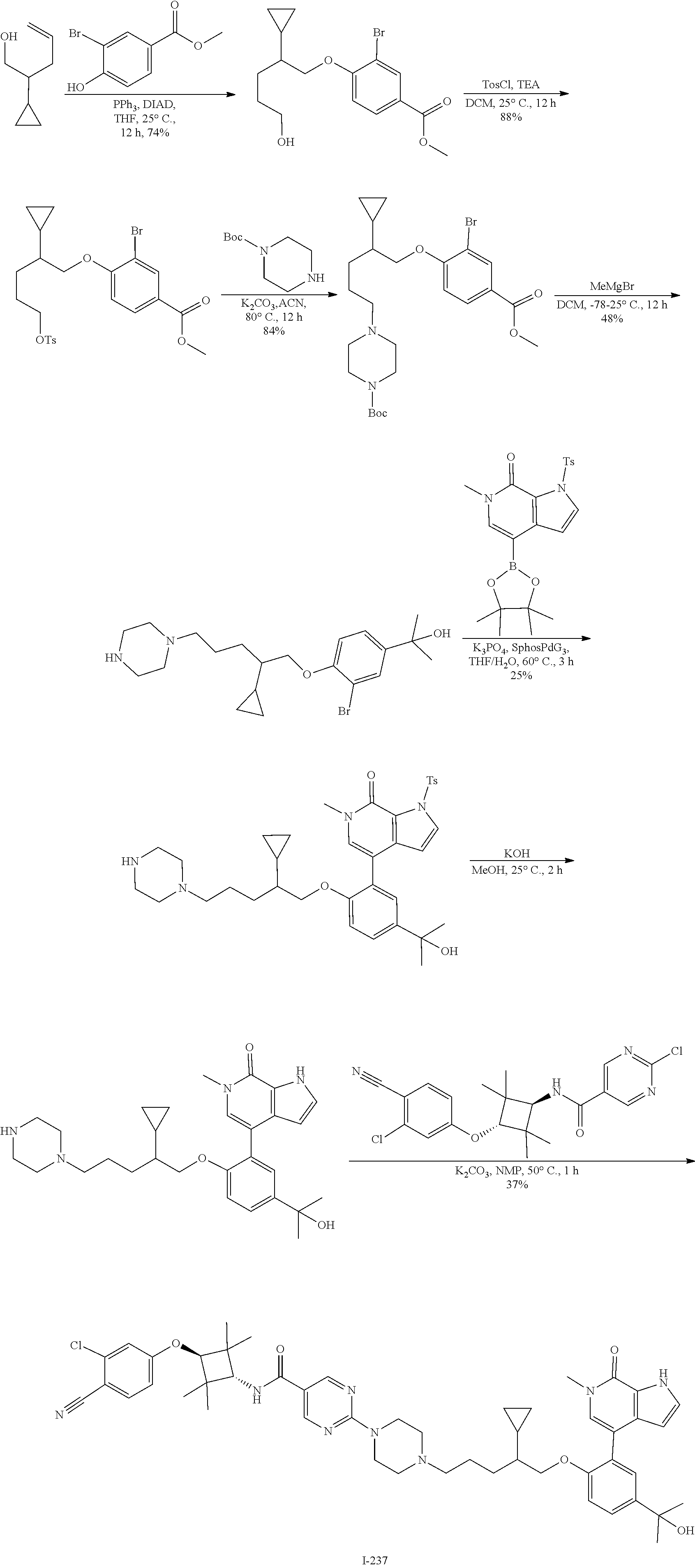

Step 1: Preparation of methyl 3-bromo-4-((2-cyclopropylpent-4-en-1-yl)oxy)benzoate. To a solution of 2-cyclopropylpent-4-en-1-ol (950 mg, 7.53 mmol, 1.0 equiv) in THF (10 mL) was added methyl 3-bromo-4-hydroxybenzoate (2.61 g, 11.3 mmol, 1.5 equiv), PPh₃ (3.95 g, 15.1 mmol, 2.0 equiv) and DIAD (3.04 g, 15.1 mmol, 2.9 mL, 2 equiv). The mixture was stirred at 25° C. for 12 h. To the reaction mixture was added to water (50 mL) and the mixture was extracted with EtOAc (50 mL). The combined organic phase was washed with brine (50 mL×3), dried over anhydrous Na₂SO₄, filtered and concentrated in vacuum. The crude product was purified by column chromatography on silica gel (petroleum ether: ethyl acetate=20:1 to 10:1) to give methyl 3-bromo-4-((2-cyclopropylpent-4-en-1-yl)oxy)benzoate (1.9 g, 5.6 mmol, 74% yield) as a yellow oil.

Step 2: Preparation of methyl 3-bromo-4-((2-cyclopropyl-5-hydroxypentyl) oxy)benzoate. To 9-BBN (0.5 M, 32 mL, 3.0 equiv) in THF (12 mL) was added to methyl 3-bromo-4-((2-cyclopropylpent-4-en-1-yl)oxy)benzoate (1.8 g, 5.3 mmol, 1.0 equiv) under the atmosphere of N₂ at 0° C. and the mixture was stirred at 25° C. for 4 h. Then BH₈NaO₇ (3.27 g, 21.2 mmol, 4.0 equiv) and H₂O (8 mL) were added and the mixture was stirred at 25° C. for 12 h. To the reaction mixture was added water (50 mL) and the mixture was extracted with EtOAc (50 mL). The combined organic phase was washed with brine (50 mL×3), dried over anhydrous Na₂SO₄, filtered and concentrated in vacuum. The residue was purified by column chromatography (SiO₂, Petroleum ether/ethyl acetate=20:1 to 2:1) to give methyl 3-bromo-4-((2-cyclopropyl-5-hydroxypentyl)oxy)benzoate (1.5 g, 4.2 mmol, 79% yield) as a colorless solid.

Step 3: Preparation of methyl 3-bromo-4-((2-cyclopropyl-5-(tosyloxy) pentyl)oxy)benzoate. To a solution of methyl 3-bromo-4-((2-cyclopropyl-5-hydroxypentyl) oxy)benzoate (1.5 g, 1.2 mmol, 1.0 equiv) in DCM (10 mL) was added TosCl (1.6 g, 8.4 mmol, 2.0 equiv) and TEA (1.3 g, 12.6 mmol, 3.0 equiv). The mixture was stirred at 25° C. for 12 h. To the reaction mixture was added water (50 mL) and the mixture was extracted with EtOAc (50 mL). The combined organic phase was washed with brine (50 mL×3), dried over anhydrous Na₂SO₄, filtered and concentrated in vacuum. The residue was purified by column chromatography (SiO₂, petroleum ether: ethyl acetate=20:1 to 6:1) to give methyl 3-bromo-4-((2-cyclopropyl-5-(tosyloxy)pentyl)oxy)benzoate (1.9 g, 3.7 mmol, 88% yield) as a yellow solid.

Step 4: Preparation of tert-butyl 4-(5-(2-bromo-4-(methoxycarbonyl)phenoxy)-4-cyclopropylpentyl)piperazine-1-carboxylate. To a solution of methyl 3-bromo-4-((2-cyclopropyl-5-(tosyloxy)pentyl)oxy)benzoate (1.85 g, 3.62 mmol, 1.0 equiv), tert-butyl piperazine-1-carboxylate (673 mg, 3.62 mmol, 1.0 equiv) in ACN (20 mL) was added K₂CO₃(1.50 g, 10.9 mmol, 3.0 equiv) and the mixture was stirred at 80° C. for 12 h. The reaction mixture was diluted with H₂O(50 mL) and extracted with EA (50 mL*2). The combined organic layers were dried over Na₂SO₄, filtered and concentrated under reduced pressure to give a residue. The residue was purified by prep-HPLC (column: Phenomenex luna C18 150 mm*40 mm*15 μm; mobile phase: [water(FA)-ACN]; B %: 20%-50%, 15 min) to afford tert-butyl 4-(5-(2-bromo-4-(methoxycarbonyl)phenoxy)-4-cyclopropylpentyl) piperazine-1-carboxylate (1.6 g, 3.0 mmol, 84% yield) as a yellow oil.

Step 5: Preparation of 2-(3-bromo-4-((2-cyclopropyl-5-(piperazin-1-yl)pentyl)oxy)phenyl)propan-2-ol. To a solution of tert-butyl 4-(5-(2-bromo-4-(methoxycarbonyl)phenoxy)-4-cyclopropylpentyl)piperazine-1-carboxylate (500 mg, 951 μmol, 1.0 equiv) in DCM (10 mL) was added MeMgBr (3 M, 4.8 mL, 15.0 equiv) at −78° C. The mixture was stirred at 25° C. for 12 h. The reaction mixture was quenched by addition saturated ammonium chloride 10 mL at 0° C., and then was diluted with H₂O (50 mL) and extracted with EA (50 mL*2). The combined organic layers were dried over Na₂SO₄, filtered and concentrated under reduced pressure to give a residue. The residue was purified by prep-HPLC (column: Phenomenex Luna C18 150 mm*25 mm*10 μm; mobile phase: [water(FA)-ACN]; B %: 15%-45%, 10 min) to afford 2-(3-bromo-4-((2-cyclopropyl-5-(piperazin-1-yl)pentyl)oxy)phenyl)propan-2-ol (200 mg, 459 4 mol, 48% yield) as a yellow gum.

Step 6: Preparation 4-(2-((2-cyclopropyl-5-(piperazin-1-yl)pentyl)oxy)-5-(2-hydroxypropan-2-yl)phenyl)-6-methyl-1-tosyl-1,6-dihydro-7H-pyrrolo[2,3-c]pyridin-7-one. To a solution of 2-(3-bromo-4-((2-cyclopropyl-5-(piperazin-1-yl)pentyl)oxy)phenyl) propan-2-ol (200 mg, 470 mol, 1.0 equiv), 6-methyl-4-(4,4,5,5-tetramethyl-1,3,2-dioxaborolan-2-yl)-1-tosyl-1,6-dihydro-7H-pyrrolo[2,3-c]pyridin-7-one (302 mg, 705 mol, 1.5 equiv) in THF (3 mL) was added SPhosPdG₃ (73 mg, 94 μmol, 0.2 equiv) and K₃PO₄ (299 mg, 1.41 mmol, 3.0 equiv) in H₂O (0.6 mL). The mixture was stirred at 60° C. for 3 h. The reaction mixture was filtered and concentrated under reduced pressure to give a residue. The residue was purified by prep-HPLC (column: UniSil 3-100 C18 Ultra (150 mm*25 mm* 3 pm); mobile phase: [water(FA)-ACN]; B %: 20%-40%, 7 min) to afford 4-(2-((2-cyclopropyl-5-(piperazin-1-yl)pentyl)oxy)-5-(2-hydroxypropan-2-yl)phenyl)-6-methyl-1-tosyl-1,6-dihydro-7H-pyrrolo[2,3-c]pyridin-7-one (75 mg, 116 μmol, 25% yield) as a white solid.

Step 7: Preparation of 4-(2-((2-cyclopropyl-5-(piperazin-1-yl)pentyl)oxy)-5-(2-hydroxypropan-2-yl)phenyl)-6-methyl-1,6-dihydro-7H-pyrrolo[2,3-c]pyridin-7-one. To a solution of compound 4-(2-((2-cyclopropyl-5-(piperazin-1-yl)pentyl)oxy)-5-(2-hydroxy-propan-2-yl)phenyl)-6-methyl-1-tosyl-1,6-dihydro-7H-pyrrolo[2,3-c]pyridin-7-one (75 mg, 116 μmol, 1.0 equiv) in MeOH (2 mL) was added KOH (1 M, 2 mL, 17.2 equiv). The mixture was stirred at 25° C. for 2 h. The reaction mixture was added CH₃COOH to adjust pH=3˜4. Freeze-drying afforded the compound 4-(2-((2-cyclopropyl-5-(piperazin-1-yl)pentyl)oxy)-5-(2-hydroxypropan-2-yl)phenyl)-6-methyl-1,6-dihydro-7H-pyrrolo[2,3-c]pyridin-7-one (57 mg, crude) as a colorless gum and it was used in the next step directly.

Step 8: Preparation of N-3-(3-chloro-4-cyanophenoxy)-2,2,4,4-tetramethylcyclobutyl)-2-(4-(4-cyclopropyl-5-(4-(2-hydroxypropan-2-yl)-2-(6-methyl-7-oxo-6,7-dihydro-1H-pyrrolo[2,3-c]pyridin-4-yl)phenoxy)pentyl)piperazin-1-yl)pyrimidine-5-carboxamide. To a solution of compound 4-(2-((2-cyclopropyl-5-(piperazin-1-yl)pentyl)oxy)-5-(2-hydroxypropan-2-yl)phenyl)-6-methyl-1,6-dihydro-7H-pyrrolo[2,3-c]pyridin-7-one (57 mg, 116 mol, 1.0 equiv) in NMP (1 mL) was added the compound 2-chloro-N—(-3-(3-chloro-4-cyanophenoxy)-2,2,4,4-tetramethylcyclobutyl) pyrimidine-5-carboxamide (49 mg, 116 mol, 1.0 equiv) and K₂CO₃ (48.0 mg, 347 μmol, 3.0 equiv). The mixture was stirred at 50° C. for 1 h. The reaction mixture was filtered and concentrated under reduced pressure to give a residue. The residue was purified by prep-HPLC (column: Phenomenex C18 75 mm*30 mm*3 um; mobile phase: [water(FA)-ACN]; B %: 28%-58%, 7 min) to afford N-3-(3-chloro-4-cyanophenoxy)-2,2,4,4-tetramethyl-cyclobutyl)-2-(4-(4-cyclopropyl-5-(4-(2-hydroxypropan-2-yl)-2-(6-methyl-7-oxo-6,7-dihydro-1H-pyrrolo [2,3-c]pyridin-4-yl)phenoxy)pentyl)piperazin-1-yl)pyrimidine-5-carboxamide (37.7 mg, 43.0 mol, 37% yield) as an off-white solid. LC-MS: MS (ES⁺): RT=1.980 min, m/z=875.4 [M+H⁺]; LCMS method: 25. ¹H NMR: (400 MHz, CD₃OD) 8.91-8.68 (m, 2H), 7.76-7.67 (m, 1H), 7.55-7.50 (m, 1H), 7.49-7.43 (m, 1H), 7.33-7.27 (m, 1H), 7.20-7.18 (m, 1H), 7.14-7.10 (m, 1H), 7.08-7.04 (m, 1H), 7.01-6.95 (m, 1H), 6.32-6.10 (m, 1H), 4.71-4.46 (m, 3H), 4.31-4.25 (m, 1H), 4.19-4.11 (m, 1H), 4.09-4.01 (m, 1H), 3.93-3.88 (m, 4H), 3.71-3.65 (m, 3H), 2.64-2.46 (m, 4H), 2.39-2.22 (m, 3H), 1.58-1.53 (m, 6H), 1.47-1.38 (m, 2H), 1.31-1.18 (m, 13H), 0.94-0.77 (m, 1H), 0.67-0.34 (m, 3H), 0.12-0.11 (m, 2H)

### Example 40—Synthesis of N-[3-(3-chloro-4-cyano-phenoxy)-2,2,4,4-tetramethyl-cyclobutyl]-2-[4-[3-[3-[2-(6-methyl-7-oxo-1H-pyrrolo[2,3-c]pyridin-4-yl)phenoxy]phenyl]propyl]piperazin-1-yl]pyrimidine-5-carboxamide (I-243)

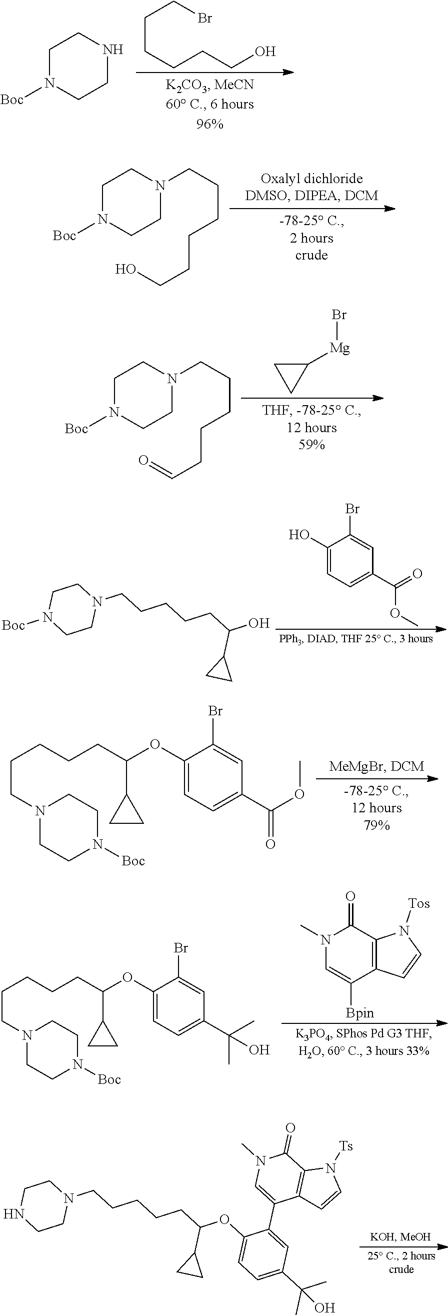

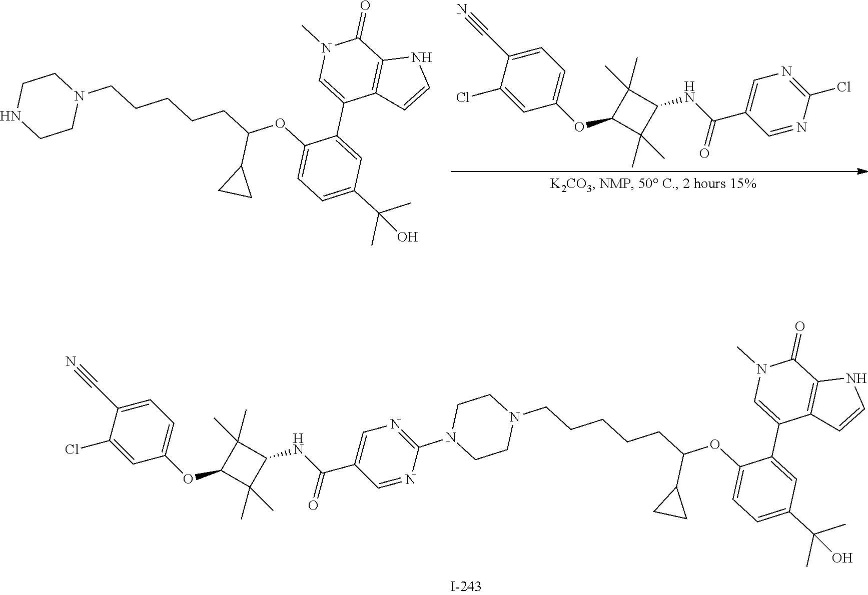

Step 1: Preparation of tert-butyl 4-(6-hydroxyhexyl) piperazine-1-carboxylate. To a solution of tert-butyl piperazine-1-carboxylate;hydrochloride (10.0 g, 44.90 mmol, 1.00 equiv) in MeCN (100 mL) was added K₂CO₃ (18.6 g, 135 mmol, 3.00 equiv) and 6-bromohexan-1-ol (9.76 g, 53.9 mmol, 7.07 mL, 1.20 equiv). The mixture was stirred at 60° C. for 6 hours. The reaction mixture was filtered and the filtrate was concentrated. The residue was purified by prep-HPLC (column: Kromasil Eternity XT 250 mm*80 mm*10 μm; mobile phase: [water (ammonia hydroxide v/v)-ACN]; B %: 23%) to give tert-butyl 4-(6-hydroxyhexyl) piperazine-1-carboxylate (12.4 g, 43.3 mmol, 96% yield) as a colorless oil.

Step 2: Preparation of tert-butyl 4-(6-oxohexylpiperazine-1-carboxylate. DMSO (1.43 g, 18.26 mmol, 1.43 mL, 5.23 equiv) was slowly added to a solution oxalyl dichloride (1.38 g, 10.9 mmol, 951 μL, 3.11 equiv) in distilled DCM (15 mL) at −78° C., and the mixture was stirred at −78° C. for 0.5 hour. Then tert-butyl 4-(6-hydroxyhexyl) piperazine-1-carboxylate (1.00 g, 3.49 mmol, 1.00 equiv) was added and the mixture was stirred at -78° C. for 1 hour. There after DIPEA (3.18 g, 24.58 mmol, 4.28 mL, 7.04 equiv) was slowly added at −78° C., and the mixture was stirred at 25° C. for 0.5 hour. The reaction mixture was concentrated to give crude tert-butyl 4-(6-oxohexylpiperazine-1-carboxylate (990 mg, crude) as a colorless oil.

Step 3: Preparation of tert-butyl 4-(6-cyclopropyl-6-hydroxy-hexyl) piperazine-1-carboxylate. To a solution of tert-butyl 4-(6-oxohexyl)piperazine-1-carboxylate (0.99 g, 3.48 mmol, 1.00 equiv) in DCM (15 mL) was added bromo(cyclopropyl)magnesium (0.5 M, 142.59 mL, 20.5 equiv) at −78° C. The mixture was stirred at 25° C. for 12 hours. The reaction mixture was quenched by addition of water 50 mL at 25° C., and then diluted with ethyl acetate 50 mL and extracted with ethyl acetate (50 mL*3). The combined organic layers were washed with brine 50 mL, dried over Na₂SO₄, and the residue was purified by prep-HPLC (column: Shim-pack C18 150 mm*25 mm*10 μm; mobile phase: [water(FA)-ACN];B %: 5%-35%, 10 nmn) to give tert-butyl 4-(6-cyclopropyl-6-hydroxy-hexyl)piperazine-1-carboxylate (670 mg, 2.05 mmol, 59% yield) as a yellow oil.

Step 4: Preparation of tert-butyl 4-[6-(2-bromo-4-methoxycarbonyl-phenoxy)-6-cyclopropyl-hexyl]piperazine-1-carboxylate. To a solution of tert-butyl 4-(6-cyclopropyl-6-hydroxy-hexvl)piperazine-1-carboxylate (1.00 g, 3.06 mmol, 1.00 equiv), methyl 3-bromo-4-hydroxy-benzoate (708 mg, 3.06 mmol, 1.00 equiv), PPh3 (2.41 g, 9.19 mmol, 3.00 equiv) in THF (10 mL) was added DIAD (1.86 g, 9.19 mmol, 1.79 mL, 3.00 equiv) at 0° C., then it was stirred at 25° C. for 3 hours. The reaction mixture was concentrated under reduced pressure to remove the solvent. The residue was diluted with ethyl acetate (200 mL) and washed with H₂O (50 mL*3), dried over anhydrous Na₂SO₄, filtered and concentrated under reduced pressure to give a residue. The residue was purified by prep-HPLC (column: Waters Xbridge C18 150 mm*50 mm*10 μm; mobile phase: [water(NH₄HCO₃)-ACN]; B %: 64%-94%, 10 min) to give tert-butyl 4-[6-(2-bromo-4-methoxycarbonyl-phenoxy)-6-cyclopropyl-hexyl]piperazine-1-carboxylate (350 mg, 649 μmol, 21% yield) as a colorless oil.

Step 5: Preparation of 2-[3-bromo-4-(1-cyclopropyl-6-piperazin-1-yl-hexoxy)phenyl]propan-2-ol. To a solution of tert-butyl 4-[6-(2-bromo-4-methoxycarbonyl-phenoxy)-6-cyclopropyl-hexyl]piperazine-1-carboxylate (0.1 M, 4.63 mL, 1.00 equiv) in DCM (4.6 mL) was added MeMgBr (3 M, 2.32 mL, 15.0 equiv) at −78° C. The mixture was stirred at 25° C. for 12 hours. The reaction mixture was quenched by the addition of NH₄Cl (1 mL) at 0° C. The reaction mixture was diluted with H₂O (5 mL) and extracted with DCM (50 mL*2). The combined organic layers were dried over Na₂SO₄, filtered and concentrated under reduced pressure to give a residue. The residue was purified by prep-HPLC (column: Phenomenex luna C18 150 mm*25 mm*10 μm; mobile phase: [water(FA)-ACN]; B %: 10%-40%, 10 min) to give 2-[3-bromo-4-(1-cyclopropyl-6-piperazin-1-yl-hexoxy)phenyl]propan-2-ol (160 mg, 364 mol, 79% yield) as a colorless oil.

Step 6: Preparation of 4-[2-(1-cyclopropyl-6-piperazin-1-yl-hexoxy)-5-(1-hydroxy-1-methyl-ethyl)phenyl]-6-methyl-1-(p-tolylsulfonyl)pyrrolo[2,3-c]pyridin-7-one. To a solution of 2-[3-bromo-4-(1-cyclopropyl-6-piperazin-1-yl-hexoxy)phenyl]propan-2-ol (160 mg, 364 μmol, 1.00 equiv) in THF (2 mL) and H₂O (0.4 mL) was added K₃PO₄ (232 mg, 1.09 mmol, 3.00 equiv), 4,6-dimethyl-1-(p-tolylsulfonyl)pyrrolo[2,3-c]pyridin-7-one (138 mg, 437 μmol, 1.20 equiv) and SPhos Pd G3 (28.4 mg, 36.4 μmol, 0.100 equiv). The mixture was stirred at 60° C. for 3 hours. The reaction mixture was filtered and the filtrate was concentrated. The organic phase was purified by prep-HPLC (column: Phenomenex luna C18 150 mm*25 mm*10 μm; mobile phase: [water(FA)-ACN]; B %: 14%-44%, min) to give 4-[2-(1-cyclopropyl-6-piperazin-1-yl-hexoxy)-5-(1-hydroxy-1-methyl-ethyl)phenyl]-6-methyl-1-(p-tolylsulfonyl)pyrrolo[2,3-c]pyridin-7-one (80.0 mg, 121 mol, 33% yield) as a white solid.

Step 7: Preparation of 4-[2-(1-cyclopropyl-6-piperazin-1-yl-hexoxy)-5-(1-hydroxy-1-methyl-ethyl) phenyl]-6-methyl-1H-pyrrolo [2,3-c]pyridin-7-one. To a solution of 4-[2-(1-cyclopropyl-6-piperazin-1-yl-hexoxy)-5-(1-hydroxy-1-methyl-ethyl) phenyl]-6-methyl-1-(p-tolylsulfonyl)pyrrolo[2,3-c]pyridin-7-one (40.0 mg, 60.5 μmol, 1.00 equiv) in MeOH (0.5 mL) was added KOH (1 M, 908 μL, 15.0 equiv). The mixture was stirred at 25° C. for 2 hours. The reaction mixture was filtered and the filtrate was concentrated to give 4-[2-(1-cyclopropyl-6-piperazin-1-yl-hexoxy)-5-(1-hydroxy-1-methyl-ethyl) phenyl]-6-methyl-1H-pyrrolo [2,3-c]pyridin-7-one (30.0 mg, crude) as a white solid.

Step 8: Preparation of N-[3-(3-chloro-4-cyano-phenoxy)-2,2,4,4-tetramethyl-cyclobutyl]-2-[4-[6-cyclopropyl-6-[4-(1-hydroxy-1-methyl-ethyl)-2-(6-methyl-7-oxo-1H-pyrrolo[2,3-c]pyridin-4-yl)phenoxy]hexyl]piperazin-1-yl]pyrimidine-5-carboxamide. To a solution of 4-[2-(1-cyclopropyl-6-piperazin-1-yl-hexoxy)-5-(1-hydroxy-1-methyl-ethyl)phenyl]-6-methyl-1H-pyrrolo[2,3-c]pyridin-7-one (30.0 mg, 59.2 mol, 1.00 equiv) in NMP (1 mL) was added K₂CO₃ (81.8 mg, 592 μmol, 10.0 equiv) and 2-chloro-N-[3-(3-chloro-4-cyano-phenoxy)-2,2,4,4-tetramethyl-cyclobutyl]pyrimidine-5-carboxamide (24.8 mg, 59.2 mol, 1.00 equiv). The mixture was stirred at 50° C. for 2 hours. The reaction mixture was filtered and purified by prep-HPLC(column: Phenomenex luna C18 150*25 mm*10 μm; mobile phase: [water(FA)-ACN]; B %: 31%-61%, 10 min) to give N-[3-(3-chloro-4-cyano-phenoxy)-2,2,4,4-tetramethyl-cyclobutyl]-2-[4-[6-cyclopropyl-6-[4-(1-hydroxy-1-methyl-ethyl)-2-(6-methyl-7-oxo-1H-pyrrolo[2,3-c]pyridin-4-yl)phenoxy]hexyl]piperazin-1-yl]pyrimidine-5-carboxamide (8.00 mg, 8.99 mol, 15.2% yield) as a white solid. ¹H NMR (400 MHz, CD₃OD): 8.67-8.63 (m, 2H), 7.62-7.57 (m, 1H), 7.35 (d, J=7.3 Hz, 1H), 7.32-7.27 (m, 1H), 7.25-7.19 (m, 2H), 7.10-7.06 (m, 1H), 7.02-6.98 (m, 1H), 6.96-6.91 (m, 1H), 6.86 (dd, J=2.3, 8.6 Hz, 1H), 6.19-6.14 (s, 1H), 4.52-4.41 (m, 2H), 4.15 (s, 1H), 4.02 (s, 1H), 3.92-3.83 (s, 3H), 3.60-3.57 (s, 3H), 2.61-2.52 (m, 4H), 2.35-2.28 (m, 1H), 1.93-1.89 (m, 2H), 1.43 (m, 4H), 1.36-1.27 (m, 2H), 1.18-1.14 (m, 6H), 1.11-1.03 (m, 12H), 0.81-0.70 (m, 1H), 0.32-0.20 (m, 2H), 0.04-0.04 (m, 2H). LC-MS: MS (ES⁺): RT=2.053 min, m/z=889.4 [M+H⁺]; LCMS method: 25.

### Example 41—Synthesis of N-[3-(3-chloro-4-cyano-phenoxy)-2,2,4,4-tetramethyl-cyclobutyl]-2-[4-[3-[3-[2-(6-methyl-7-oxo-1H-pyrrolo[2,3-c]pyridin-4-yl)-4-methyl-sulfonyl-phenoxy]phenyl]propyl]-1-piperidyl]pyrimidine-5-carboxamide (I-249)

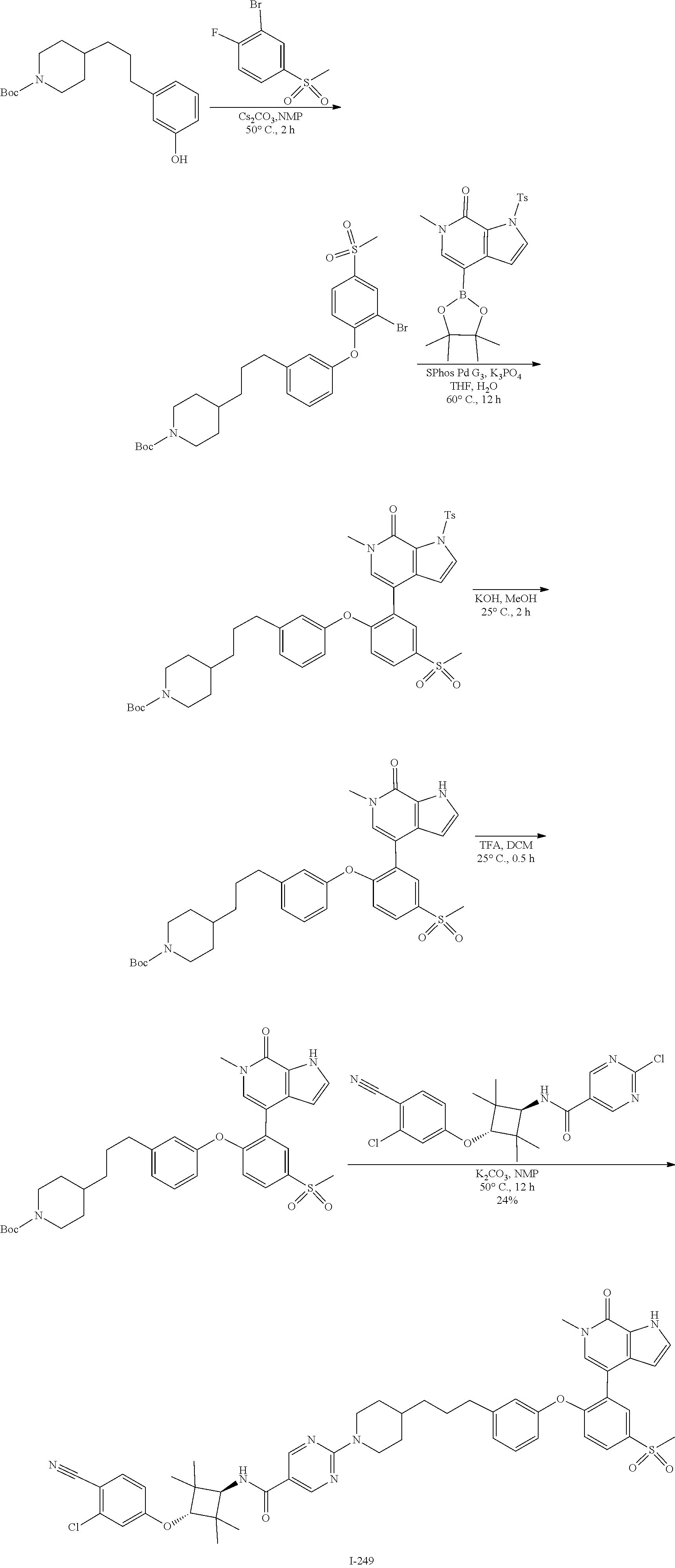

Step 1: Preparation of tert-butyl 4-13-[3-(2-bromo-4-methylsulfonyl-phenoxy)phenyl]propyl]piperidine-1-carboxylate. To a solution of tert-butyl 4-[3-(3-hydroxyphenyl)propyl]piperidine-1-carboxylate (500 mg, 1.5 mmol, 1 equiv) and 2-bromo-1-fluoro-4-methylsulfonyl-benzene (396 mg, 1.5 mmol, 1 equiv) in NMP (3 mL) was added Cs₂CO₃ (1.53 g, 4.7 mmol, 3 equiv). The mixture was stirred at 50° C. for 2 h. The residue was diluted with H₂O (50 mL) and extracted with EA (50 mL*3). The combined organic layers were washed with sat. NaCl (50 mL*3), dried over Na₂SO₄, filtered and concentrated. The residue was purified by column chromatography (SiO₂, petroleum ether: ethyl acetate=50:1 to 3:1) to give tert-butyl 4-[3-[3-(2-bromo-4-methylsulfonyl-phenoxy) phenyl]propyl]piperidine-1-carboxylate (862 mg, 99% yield).

Step 2: Preparation of tert-butyl 4-[3-[3-[2-[6-methyl-7-oxo-1-(p-tolylsulfonyl)pyrrolo[2,3-c]pyridin-4-yl]-4-methylsulfonyl-phenoxy]phenyl]propyl]piperidine-1-carboxylate. To a solution of 6-methyl-1-(p-tolylsulfonyl)-4-(4,4,5,5-tetramethyl-1,3,2-dioxaborolan-2-yl)pyrrolo[2,3-c]pyridin-7-one (325 mg, 760 mol, 1.2 equiv) and tert-butyl 4-[3-[3-(2-bromo-4-methylsulfonyl-phenoxy)phenyl]propyl]piperidine-1-carboxylate (350 mg, 633 μmol, 1 equiv) in THF (10 mL) and H₂O (2 mL) was added K₃PO₄ (403 mg, 1.9 mmol, 3 equiv) and SPhos Pd G3 (49 mg, 63 μmol, 0.1 equiv). The mixture was stirred at 60° C. for 12 h. The mixture was filtered and the residue was diluted with H₂O (20 mL) and extracted with EA (20 mL). The combined organic layers were washed with sat. NaCl (20 mL*3), dried over Na₂SO₄, and concentrated. The residue was purified by prep-TLC (SiO₂, petroleum ether: ethyl acetate=4:1) to give tert-butyl 4-[3-[3-[2-[6-methyl-7-oxo-1-(p-tolylsulfonyl)pyrrolo[2,3-c]pyridin-4-yl]-4-methylsulfonyl-phenoxy]phenyl]propyl]piperidine-1-carboxylate (282 mg, 57% yield).

Step 3: Preparation of tert-butyl 4-[3-[3-[2-(6-methyl-7-oxo-1H-pyrrolo [2,3-c]pyridin-4-yl)-4-methylsulfonyl-phenoxy]phenyl]propyl]piperidine-1-carboxylate. To a solution of tert-butyl 4-[3-[3-[2-[6-methyl-7-oxo-1-(p-tolylsulfonyl) pyrrolo[2,3-c]pyridin-4-yl]-4-methylsulfonyl-phenoxy]phenyl]propyl]piperidine-1-carboxylate (282 mg, 364.3 μmol, 1 equiv) in MeOH (5 mL) was added KOH (204 mg, 3.6 mmol, 10 equiv). The mixture was stirred at 25° C. for 2 h. The residue was diluted with H₂O (20 mL) and extracted with EA (20 mL*3). The combined organic layers were washed with sat. NaCl (20 mL*3), dried over Na₂SO₄, filtered and concentrated. The residue was purified by prep-TLC (SiO₂, petroleum ether: ethyl acetate=1:4) to give tert-butyl 4-[3-[3-[2-(6-methyl-7-oxo-1H-pyrrolo[2,3-c]pyridin-4-yl)-4-methylsulfonyl-phenoxy]phenyl]propyl]piperidine-1-carboxylate (124 mg, 55% yield).

Step 4: Preparation of 6-methyl-4-[5-methylsulfonyl-2-[3-[3-(4-piperidyl) propyl]phenoxy]phenyl]-1H-pyrrolo[2,3-c]pyridin-7-one. To a solution of tert-butyl 4-[3-[3-[2-(6-methyl-7-oxo-1H-pyrrolo[2,3-c]pyridin-4-yl)-4-methylsulfonyl-phenoxy]phenyl]propyl]piperidine-1-carboxylate (62 mg, 100 μmol, 1 equiv) in DCM (2 mL) was added TFA (1 mL). The mixture was stirred at 25° C. for 0.5 h. The mixture was filtered and concentrated to give 6-methyl-4-[5-methylsulfonyl-2-[3-[3-(4-piperidyl)propyl]phenoxy]phenyl]-1H-pyrrolo[2,3-c]pyridin-7-one (103 mg, crude) as a residue, which was used in the next step without further purification.

Step 5: Preparation of N-[3-(3-chloro-4-cyano-phenoxy)-2,2,4,4-tetramethyl-cyclobutyl]-2-[4-[3-[3-[2-(6-methyl-7-oxo-1H-pyrrolo[2,3-c]pyridin-4-yl)-4-methyl-sulfonyl-phenoxy]phenyl]propyl]-1-piperidyl]pyrimidine-5-carboxamide. To a solution of 6-methyl-4-[5-methylsulfonyl-2-[3-[3-(4-piperidyl)propyl]phenoxy]phenyl]-1H-pyrrolo[2,3-c]pyridin-7-one (103 mg, 198 mol, 1 equiv) and 2-chloro-N-[3-(3-chloro-4-cyano-phenoxy)-2,2,4,4-tetramethyl-cyclobutyl]pyrimidine-5-carboxamide (83 mg, 198 μmol, 1 equiv) in NMP (2 mL) was added K₂CO₃ (137 mg, 991 mol, 5 equiv). The mixture was stirred at 50° C. for 12 h. The residue was purified by prep-HPLC (column: Waters Xbridge C18 150 mm*50 mm*10 μm; mobile phase: [water (NH₄HCO₃)-ACN]; B %: 68%-98%, 9 min) to give the desired product N-[3-(3-chloro-4-cyano-phenoxy)-2,2,4,4-tetramethyl-cyclobutyl]-2-[4-[3-[3-[2-(6-methyl-7-oxo-1H-pyrrolo[2,3-c]pyridin-4-yl)-4-methylsulfonyl-phenoxy]phenyl]propyl]-1-piperidyl]pyrimidine-5-carboxamide (43 mg, 24% yield). LC-MS: MS (ES⁺): RT=2.919 min, m/z=902.5[M+1]; LCMS method: 25. 1H NMR: ¹H NMR (400 MHz, DMSO-d₆) δ 12.14 (s, 1H), 8.77 (s, 2H), 7.98 (d, J=2.5 Hz, 1H), 7.92-7.84 (m, 2H), 7.66 (s, 1H), 7.42 (s, 1H), 7.27 (s, 2H), 7.23-7.20 (m, 1H), 7.07 (s, 1H), 7.02-6.98 (m, 2H), 6.95-6.88 (m, 2H), 6.29 (br d, J=18.0 Hz, 1H), 4.77-4.67 (m, 2H), 4.30-4.26 (m, 1H), 4.06 (br d, J=2.1 Hz, 1H), 3.61 (d, J=1.8 Hz, 3H), 3.25 (s, 3H), 2.98 (s, 2H), 2.70-2.54 (m, 3H), 2.35-2.31 (m, 1H), 1.80-1.69 (m, 2H), 1.63-1.51 (m, 3H), 1.27-1.16 (m, 9H), 1.13-1.08 (m, 6H).

### Example 42—Synthesis of N-[3-(3-chloro-4-cyano-phenoxy)-2,2,4,4-tetramethyl-cyclobutyl]-2-[4-[3-[3-[2-(6-methyl-7-oxo-1H-pyrrolo[2,3-c]pyridin-4-yl)phenoxy]phenyl]propyl]piperazin-1-yl]pyrimidine-5-carboxamide (I-253)

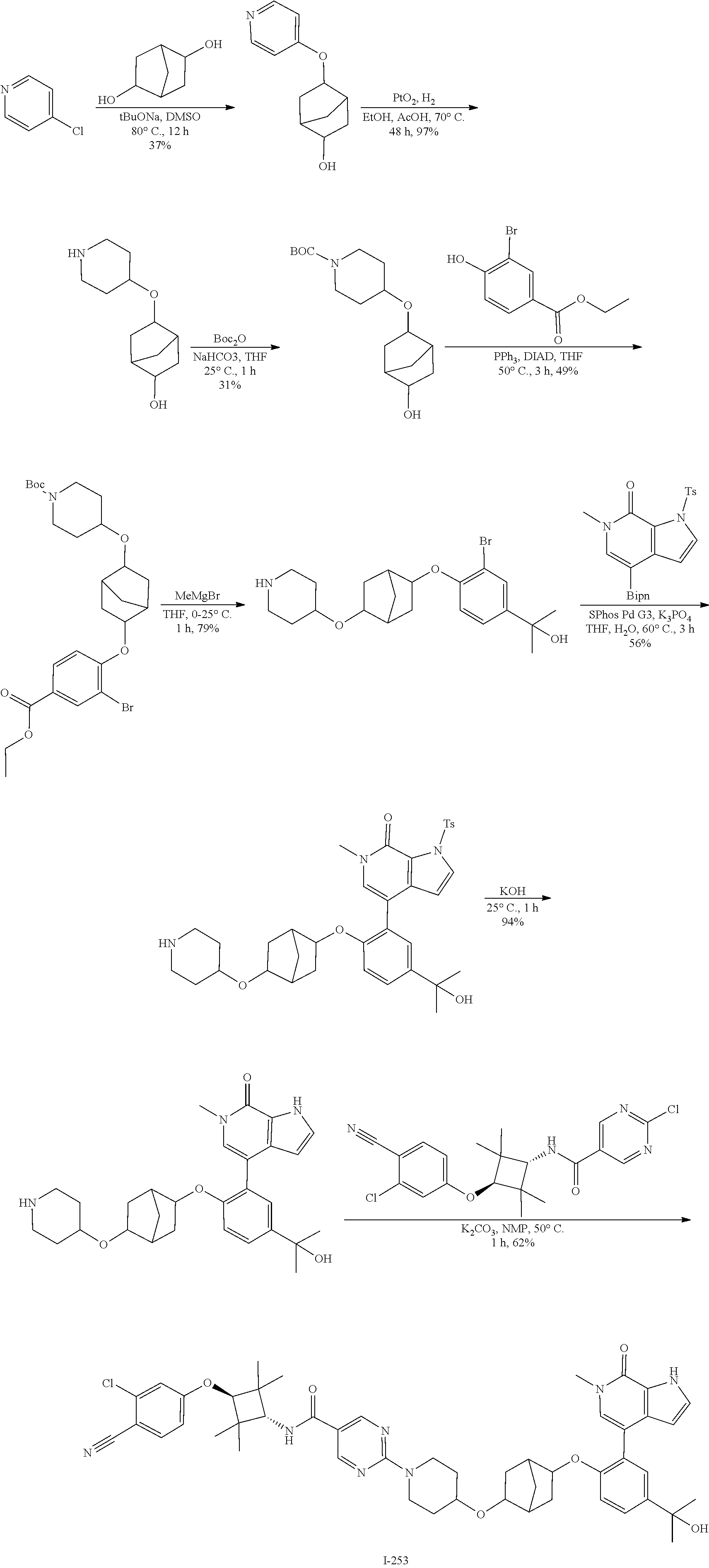

Step 1: Preparation of 5-(4-pyridyloxy)norbornan-2-ol. To a solution of 4-chloropyridine (4 g, 26.6 mmol, 1.0 equiv, HCl) and norbornane-2,5-diol (6.8 g, 53.3 mmol, 2.0 equiv) in DMSO (40 mL) was added sodium 2-methylpropan-2-olate (5.1 g, 53.3 mmol, 2.0 equiv). The mixture was stirred at 80° C. for 12 h. The reaction mixture was filtered and concentrated. The residue was purified by prep-HPLC (column: Kromasil Eternity XT 250 mm*80 mm*10 μm; mobile phase: [water (NH₄HCO₃)-ACN]; B %: 5%-35%, 20 min) to give 5-(4-pyridyloxy) norboman-2-ol (2 g, 36.5% yield).

Step 2: Preparation of 5-(4-piperidyloxy) norbornan-2-ol. To a solution of 5-(4-pyridyloxy)norboman-2-ol (2 g, 9.7 mmol, 1.0 equiv) in EtOH (20 mL) and ACOH (20 mL) was added PtO₂ (2.2 g, 9.7 mmol, 1.0 equiv) under N₂. The mixture was stirred under H₂ (50 psi) at 70° C. for 48 h. The reaction mixture was filtered and concentrated. The product was used directly for next step.

Step 3: Preparation of Tert—butyl 4-(5-hydroxynorbornan-2-yl) oxypiperidine-1-carboxylate. To a solution of 5-(4-piperidyloxy) norboman-2-ol (1 g, 4.7 mmol, 1.0 equiv) in THF (10 mL) was added Boc₂O (929.6 mg, 4.3 mmol, 0.9 equiv) and NaHCO₃ (1.2 g, 14.2 mmol, 3.0 equiv). The mixture was stirred at 25° C. for 1 h. The reaction solution is filtered and concentrated. The residue was purified by column chromatography (SiO₂, petroleum ether: ethyl acetate=2:1 to 1:1) to give Tert-butyl 4-(5-hydroxynorboman-2-yl) oxypiperidine-1-carboxylate (450 mg, 30% yield).

Step 4: Preparation of tert—butyl 4-[5-(2-bromo-4-ethoxycarbonyl-phenoxy)norbornan-2-yl]oxypiperidine-1-carboxylate. A mixture of tert-butyl 4-(5-hydroxynorboman-2-yl)oxypiperidine-1-carboxylate (380 mg, 1.2 mmol, 1.0 equiv), ethyl 3-bromo-4-hydroxy-benzoate (299 mg, 1.2 mmol, 1.0 equiv), DIAD (493.5 mg, 2.4 mmol, 2.0 equiv) and PPh₃ (0.25 M, 9.7 mL, 2.0 equiv), in THF (10 mL) was degassed and purged with N₂ three times, and then the mixture was stirred at 50° C. for 3 h under an N₂ atmosphere. The reaction mixture was filtered and the filtrate was concentrated. The residue was purified by prep-HPLC (column: Phenomenex C18 75 mm*30 mm*3 um; mobile phase: [water (FA)-ACN]; B %: 80%-100%, 7 min) to give tert—butyl 4-[5-(2-bromo-4-ethoxycarbonyl-phenoxy)norboman-2-yl]oxypiperidine-1-carboxylate (320 mg, 48% yield).

Step 5: Preparation of 2-[3-bromo-4-[5-(4-piperidyloxy) norbornan-2-yl]oxy-phenyl]propan-2-ol. A mixture of tert-butyl 4-[5-(2-bromo-4-ethoxycarbonyl-phenoxy)norboman-2-yl]oxypiperidine-1-carboxylate (320 mg, 594 mol, 1.0 equiv) MeMgBr (3 M, 3.9 mL, 20.0 equiv) in THF (5 mL) was degassed and purged with N₂ three times, and then the mixture was stirred at 25° C. for 1 h under N₂ atmosphere. The reaction mixture was filtered and the filtrate was concentrated. The residue was purified by prep-HPLC (column: Unisil 3-100 C18 Ultra 150 mm*50 mm*3 μm; mobile phase: [water(FA)-ACN]; B %: 13%-43%, 7 min) to give 2-[3-bromo-4-[5-(4-piperidyloxy) norboman-2-yl]oxy-phenyl]propan-2-ol (200 mg, 79% yield).

Step 6: Preparation of 4-[5-(1-hydroxy-1-methyl-ethyl)-2-[5-(4-piperidyloxy) norbornan-2-yl]oxy-phenyl]-6-methyl-1-(p-tolylsulfonyl) pyrrolo [2, 3-c]pyridin-7-one. To a solution of 2-[3-bromo-4-[5-(4-piperidyloxy)norbornan-2-yl]oxy-phenyl]propan-2-ol (100 mg, 235 μmol, 1.0 equiv) and 6-methyl-1-(p-tolylsulfonyl)-4-(4,4,5,5-tetramethyl-1,3,2-dioxaborolan-2-yl)pyrrolo[2,3-c]pyridin-7-one (111 mg, 259 mol, 1.1 equiv) in THF (2 mL) and H₂O (0.5 mL) was added SPhos Pd G₃ (37 mg, 47 μmol, 0.2 equiv) and K₃PO₄ (150 mg, 706 μmol, 3.0 equiv). The mixture was stirred at 60° C. for 3 h. The reaction mixture was filtered and concentrated. The residue was purified by prep-HPLC (column: Unisil 3-100 C18 Ultra 150 mm*50 mm*3 μm; mobile phase: [water(FA)-ACN]; B %: 19%-49%, 7 min) to give 4-[5-(1-hydroxy-1-methyl-ethyl)-2-[5-(4-piperidyloxy) norboman-2-yl]oxy-phenyl]-6-methyl-1-(p-tolylsulfonyl) pyrrolo [2, 3-c]pyridin-7-one (85 mg, 55.8% yield).

Step 7: Preparation of 4-[5-(1-hydroxy-1-methyl-ethyl)-2-[5-(4-piperidyloxy) norbornan-2-yl]oxy-phenyl]-6-methyl-1H-pyrrolo[2,3-c]pyridin-7-one. To a solution of 4-[5-(1-hydroxy-1-methyl-ethyl)-2-[5-(4-piperidyloxy)norboman-2-yl]oxy-phenyl]-6-methyl-1-(p-tolylsulfonyl)pyrrolo[2,3-c]pyridin-7-one (70 mg, 108 mol, 1.0 equiv) in MeOH (1 mL) was added KOH (30 mg, 541 μmol, 5.0 equiv). The mixture was stirred at 25° C. for 1 h. The reaction mixture was filtered and the filtrate was concentrated. The product was used directly for next step.

Step 8: Preparation of N-[3-(3-chloro-4-cyano-phenoxy)-2,2,4,4-tetramethyl-cyclobutyl]-2-[4-[5-[4-(1-hydroxy-1-methyl-ethyl)-2-(6-methyl-7-oxo-1H-pyrrolo[2,3-c]pyridin-4-yl)phenoxy]norbornan-2-yl]oxy-1-piperidyl]pyrimidine-5-carboxamide. To a solution of 4-[5-(1-hydroxy-1-methyl-ethyl)-2-[5-(4-piperidyloxy)norbornan-2-yl]oxy-phenyl]-6-methyl-1H-pyrrolo[2,3-c]pyridin-7-one (40 mg, 81 mol, 1.0 equiv) in NMP (1 mL) was added K₂CO₃ (11 mg, 81 μmol, 1.0 equiv) and 2-chloro-N-[3-(3-chloro-4-cyano-phenoxy)-2,2,4,4-tetramethyl-cyclobutyl]pyrimidine-5-carboxamide (34 mg, 81 mol, 1.0 equiv) The mixture was stirred at 50° C. for 1 h. The residue was purified by prep-HPLC (column: Phenomenex Luna C18 150 mm*25 mm*10 μm; mobile phase: [water (FA)-ACN]; B %: 60%-90%, 10 min) to give N-[3-(3-chloro-4-cyano-phenoxy)-2,2,4,4-tetramethyl-cyclobutyl]-2-[4-[5-[4-(1-hydroxy-1-methyl-ethyl)-2-(6-methyl-7-oxo-1H-pyrrolo[2,3-c]pyridin-4-yl)phenoxy]norboman-2-yl]oxy-1-piperidyl]pyrimidine-5-carboxamide (45 mg, 62% oyield). ¹H NMR (400 MHz, DMSO) δ 11.99 (s, 1H), 8.75 (s, 2H), 7.90 (d, J=8.8 Hz, 1H), 7.73 (d, J=9.2 Hz, 1H), 7.42 (d, J=2.4 Hz, 1H), 7.36 (s, 1H), 7.27 (t, J=2.4 Hz, 1H), 7.22 (d, J=2.4 Hz, 1H), 7.17 (s, 1H), 7.00 (s, 1H), 6.93 (d, J=8.8 Hz, 1H), 6.09 (1, J=2.4 Hz, 1H), 4.93 (s, 1H), 4.60-4.46 (m, 1H), 4.38-4.23 (m, 3H), 4.03 (d, J=9.2 Hz, 1H), 3.57 (s, 3H), 3.56-3.48 (m, 1H), 3.47-3.37 (m, 3H), 2.34 (s, 1H), 2.19 (s, 1H), 2.08-1.96 (m, 1H), 1.80 (s, 3H), 1.43 (s, 6H), 1.32-1.24 (m, 3H), 1.21 (s, 6H), 1.11 (s, 6H), 1.00-0.91 (m, 1H), 0.81-0.71 (m, 1H). LC-MS: MS (ES⁺): RT=2.803 min, m/z=874.4 [M+H⁺]; LCMS method: 25.

### Example 43—Synthesis of N-[3-(3-chloro-4-cyano-phenoxy)-2,2,4,4-tetramethyl-cyclobutyl]-2-[4-[1-[2-[4-(1-hydroxy-1-methyl-ethyl)-2-(6-methyl-7-oxo-1H-pyrrolo[2,3-c]pyridin-4-yl)phenoxy]-2-methyl-propyl]azetidin-3-yl]oxy-1-piperidyl]pyrimidine-5-carboxamide (I-254)

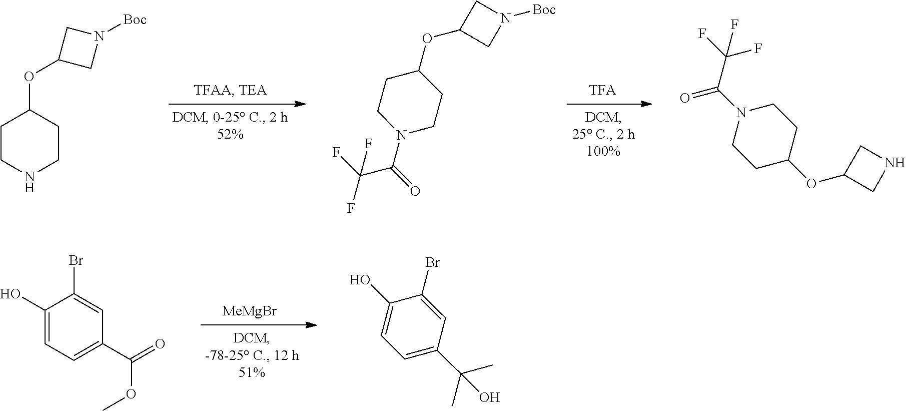 [SMILES: "MeMgBr" ; CC(C)(C)OC(=O)N1CC(OC2CCNCC2)C1 ; CC(C)(C)OC(=O)N1CC(OC2CCN(C(=O)C(F)(F)F)CC2)C1 ; O=C(N1CCC(OC2CNC2)CC1)C(F)(F)F ; COC(=O)c1ccc(O)c(Br)c1 ; CC(C)(O)c1ccc(O)c(Br)c1]

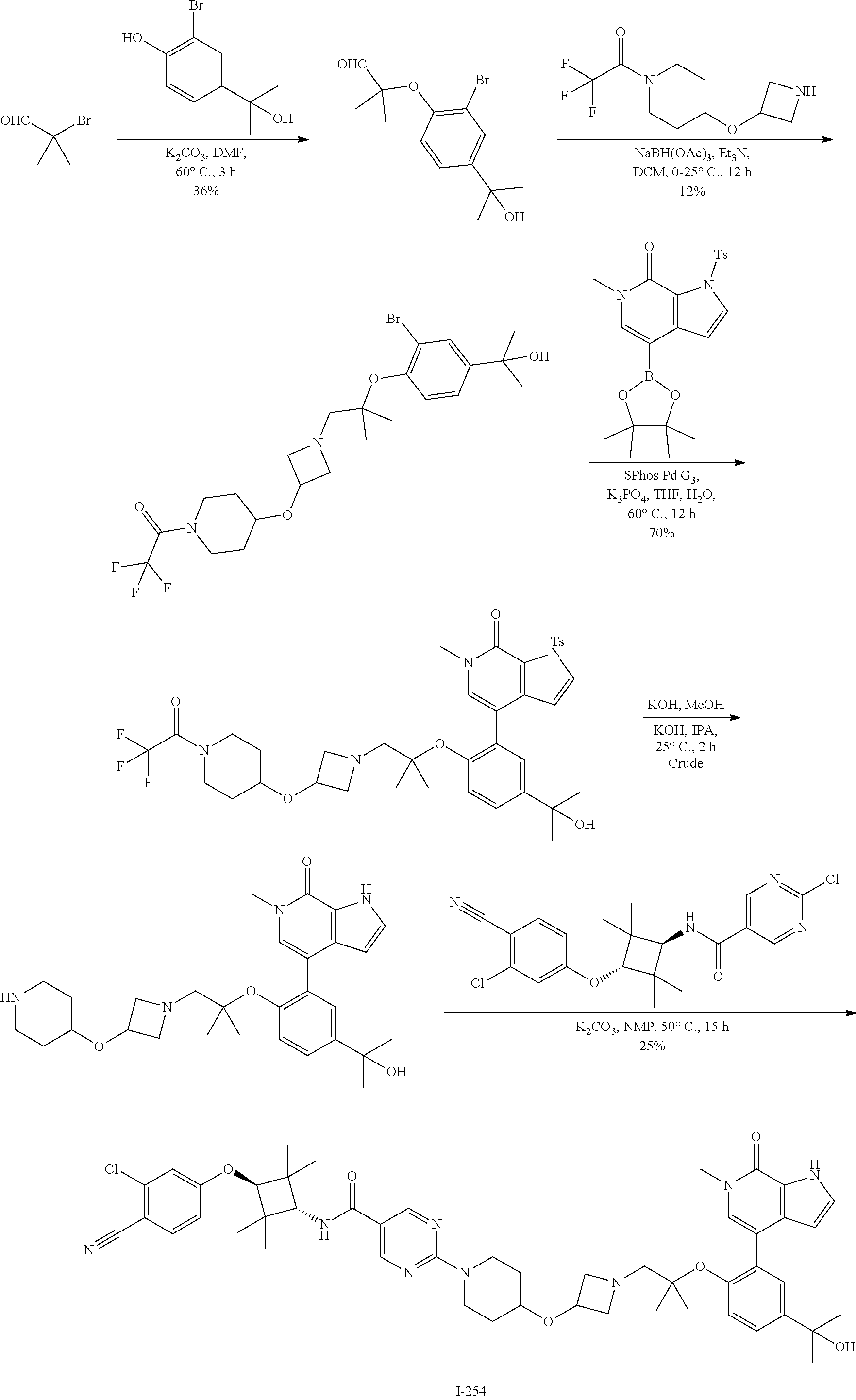

Step 1: Preparation of tert-butyl 3-[[1-(2,2,2-trifluoroacetyl)-4-piperidyl]oxy]azetidine-1-carboxylate. To a stirred solution of tert-butyl 3-(4-piperidyloxy)azetidine-1-carboxylate (2.80 g, 10.9 mmol, 1.0 equiv) in DCM (100 mL), DIEA (3.53 g, 27.3 mmol, 4.76 mL, 2.5 equiv) was added followed by the addition of TFAA (2.79 g, 13.3 mmol, 1.85 mL, 1.2 equiv) at 0° C. and the reaction mixture was stirred at 25° C. for 2 h. The reaction mixture was diluted with DCM (50 mL) and the pH was adjusted to 7.0 with 10% NaHCO₃ aq and then extracted with DCM (50 mL×3). The combined organic layers were washed with brine (50 mL), dried over Na₂SO₄, filtered and concentrated under reduced pressure to give a residue. The residue was purified by column chromatography (SiO₂, petroleum ether: ethyl acetate=10:1 to 1:1). Compound tert-butyl 3-[[1-(2,2,2-trifluoroacetyl)-4-piperidyl]oxy]azetidine-1-carboxylate (2.00 g, 5.68 mmol, 52% yield) was obtained as a white solid.

Step 2: Preparation of 1-[4-(azetidin-3-yloxy)-1-piperidyl]-2,2,2-trifluoro-ethanone. To a stirred solution of tert-butyl 3-[[1-(2,2,2-trifluoroacetyl)-4-piperidyl]oxy]azetidine-1-carboxylate (2.00 g, 5.68 mmol, 1.0 equiv) in DCM (10 mL), TFA (15.4 g, 135 mmol, 10 mL, 23.8 equiv) was added and the reaction mixture was stirred at 25° C. for 2 h. The reaction mixture was concentrated under reduced pressure to give a residue. Compound 1-[4-(azetidin-3-yloxy)-1-piperidyl]-2,2,2-trifluoro-ethanone (2.08 g, 5.68 mmol, 100% yield, TFA) was obtained as a colorless gum and was used in the next step without further purification.

Step 3: Preparation of 2-bromo-4-(1-hydroxy-1-methyl-ethyl)phenol. A mixture of methyl 3-bromo-4-hydroxy-benzoate (5.0 g, 22 mmol, 1.0 equiv) in DCM (500 mL) was degassed at −75° C. MeMgBr (3.00 M, 108 mL, 15.0 equiv) was added, the mixture purged with N₂ three times, and then the mixture was stirred at 25° C. for 12 h under an N₂ atmosphere. The crude product was purified by column chromatography on silica gel (petroleum ether: ethyl acetate=10:1 to 3:1) to give the compound 2-bromo-4-(1-hydroxy-1-methyl-ethyl)phenol (2.57 g, 11.1 mmol, 51% yield) as a white solid.

Step 4: Preparation of 2-[2-bromo-4-(1-hydroxy-1-methyl-ethyl)phenoxy]-2-methyl-propanal. A mixture of 2-bromo-2-methyl-propanal (500 mg, 3.31 mmol, 1.0 equiv), 2-bromo-4-(1-hydroxy-1-methyl-ethyl)phenol (765 mg, 3.31 mmol, 1.0 equiv), K₂CO₃(1.37 g, 9.93 mmol, 3.0 equiv) in DMF (5 mL) was degassed and purged with N₂ three times, and then the mixture was stirred at 60° C. for 3 h under an N₂ atmosphere. The crude product was purified by column chromatography on silica gel (petroleum ether: ethyl acetate=10:1 to 3:1) to give the compound 2-[2-bromo-4-(1-hydroxy-1-methyl-ethyl)phenoxy]-2-methyl-propanal (360 mg, 1.20 mmol, 36% yield) as a white solid.

Step 5: Preparation of 1-[4-[1-[2-[2-bromo-4-(1-hydroxy-1-methyl-ethyl) phenoxy]-2-methyl-propyl]azetidin-3-yl]oxy-1-piperidyl]-2,2,2-trifluoro-ethanone. To a solution of 1-[4-(azetidin-3-yloxy)-1-piperidyl]-2,2,2-trifluoro-ethanone (302 mg, 1.20 mmol, 1.0 equiv) in DCM (3 mL) was added Et₃N (605 mg, 5.98 mmol, 832 4 μL, 5.0 equiv) dropwise and 2-[2-bromo-4-(1-hydroxy-1-methyl-ethyl)phenoxy]-2-methyl-propanal (360 mg, 1.20 mmol, 1.0 equiv). After addition, the mixture was stirred at 25° C. for 0.5 h, and then NaBH(OAc)₃ (760 mg, 3.59 mmol, 3.0 equiv) was added dropwise at 0° C. The resulting mixture was stirred at 25° C. for 11.5 h. The residue was purified by preparative TLC on silica gel (petroleum ether: EtOAc=1:1) to give the compound 1-[4-[1-[2-[2-bromo-4-(1-hydroxy-1-methyl-ethyl)phenoxy]-2-methyl-propyl]azetidin-3-yl]oxy-1-piperidyl]-2,2,2-trifluoro-ethanone (80.0 mg, 149 μmol, 12% yield) was obtained as a colorless oil.

Step 6: Preparation of 4-[2-[1,1-dimethyl-2-[3-[[1-(2,2,2-trifluoroacetyl)-4-piperidyl]oxy]azetidin-1-yl]ethoxy]-5-(1-hydroxy-1-methylethyl)phenyl]-6-methyl-1-(p-tolylsulfonyl)pyrrolo[2,3-c]pyridin-7-one. A mixture of 1-[4-[1-[2-[2-bromo-4-(1-hydroxy-1-methyl-ethyl)phenoxy]-2-methyl-propyl]azetidin-3-yl]oxy-1-piperidyl]-2,2,2-trifluoro-ethanone (90.0 mg, 167 mol, 1.0 equiv), 6-methyl-1-(p-tolylsulfonyl)-4-(4,4,5,5-tetramethyl-1,3,2-dioxaborolan-2-yl)pyrrolo[2,3-c]pyridin-7-one (93.2 mg, 216 μmol, 1.3 equiv), K₃PO₄ (107 mg, 502 μmol, 3.0 equiv) and SPhos Pd G3 (26.1 mg, 33.5 mol, 0.20 equiv) in THF (3 mL) and H₂O (0.6 mL) was stirred at 60° C. for 12 h under N₂. The residue was purified by prep-TLC (dichloromethane: methyl alcohol=10:1). The compound 4-[2-[1,1-dimethyl-2-[3-[[1-(2,2,2-trifluoroacetyl)-4-piperidyl]oxy]azetidin-1-yl]ethoxy]-5-(1-hydroxy-1-methylethyl) phenyl]-6-methyl-1-(p-tolylsulfonyl)pyrrolo[2,3-c]pyridin-7-one (90.0 mg, 119 mol, 70% yield) was obtained as a white solid.

Step 7: Preparation of product 4-12-[1,1-dimethyl-2-[3-(4-piperidyloxy) azetidin-1-yl]ethoxy]-5-(1-hydroxy-1-methyl-ethyl)phenyl]-6-methyl-1H-pyrrolo[2,3-c]pyridin-7-one. To a solution of 4-[2-[1,1-dimethyl-2-[3-[[1-(2,2,2-trifluoroacetyl)-4-piperidyl]oxy]azetidin-1-yl]ethoxy]-5-(1-hydroxy-1-methyl-ethyl)phenyl]-6-methyl-1-(p-tolylsulfonyl)pyrrolo[2,3-c]pyridin-7-one (70 mg, 92. mol, 1.0 equiv) in IPA (0.5 mL) was added KOH (1.0 M, 1.4 mL, 15.0 equiv). The mixture was stirred at 25° C. for 2 h. Acetic acid was added to adjust the pH and water was added, followed by to lyophilization to afford the crude product 4-[2-[1,1-dimethyl-2-[3-(4-piperidyloxy)azetidin-1-yl]ethoxy]-5-(1-hydroxy-1-methyl-ethyl)phenyl]-6-methyl-1H-pyrrolo[2,3-c]pyridin-7-one (46 mg, crude) as a white solid, which was used in the next step without further purification.

Step 8: Preparation of N-[3-(3-chloro-4-cyano-phenoxy)-2,2,4,4-tetramethyl-cyclobutyl]-2-[4-[1-[2-[4-(1-hydroxy-1-methyl-ethyl)-2-(6-methyl-7-oxo-1H-pyrrolo[2,3-c]pyridin-4-yl)phenoxy]-2-methyl-propyl]azetidin-3-yl]oxy-1-piperidyl]pyrimidine-5-carboxamide (I-254). To a solution of 4-[2-[1,1-dimethyl-2-[3-(4-piperidyloxy)azetidin-1-yl]ethoxy]-5-(1-hydroxy-1-methyl-ethyl)phenyl]-6-methyl-¹H-pyrrolo[2,3-c]pyridin-7-one (46 mg, 90 mol, 1.0 equiv) in NMP (1 mL) was added K₂CO₃ (37.5 mg, 271 mol, 3.0 equiv) and 2-chloro-N-[3-(3-chloro-4-cyano-phenoxy)-2,2,4,4-tetramethyl-cyclobutyl]pyrimidine-5-carboxamide (37.9 mg, 90.4 mol, 1.0 equiv). The mixture was stirred at 50° C. for 12 h. The reaction mixture was concentrated in vacuo to give the crude product. The residue was purified by prep-HPLC (column: Waters Xbridge 150 mm*25 mm * Sum; mobile phase: [water(NH₄HCO₃)-ACN]; B %: 55%-85%, 8 min). The compound N-[3-(3-chloro-4-cyano-phenoxy)-2,2,4,4-tetramethyl-cyclobutyl]-2-[4-[1-[2-[4-(1-hydroxy-1-methyl-ethyl)-2-(6-methyl-7-oxo-1H-pyrrolo[2,3-c]pyridin-4-yl)phenoxy]-2-methyl-propyl]azetidin-3-yl]oxy-1-piperidyl]pyrimidine-5-carboxamide (20.9 mg, 22.9 mol, 25% yield, 98% purity) was obtained as a white solid. ¹H NMR (CD₃OD, 400 MHz):δ=8.76 (s, 2H), 7.74 (d, J=8.8 Hz, 1H), 7.58 (d, J=2.4 Hz, 1H), 7.47-7.41 (m, 1H), 7.36 (d, J=2.8 Hz, 1H), 7.25 (s, 1H), 7.19-7.13 (m, 2H), 7.00 (m, 1H), 6.33 (d, J=2.8 Hz, 1H), 4.63 (s, 1H), 4.42-4.33 (m, 2H), 4.29 (s, 1H), 4.22 (m, 1H), 4.15 (s, 1H), 3.74 (s, 3H), 3.67-3.49 (m, 5H), 2.97-2.85 (m, 2H), 2.47 (s, 2H), 1.94-1.83 (m, 2H), 1.58 (s, 6H), 1.30 (s, 6H), 1.23 (s, 6H), 1.06 (s, 6H), 0.93-0.88 (m, 2H). LC-MS: MS (ES⁺): RT=1.191 min, m/z=891.4 [M+H⁺]; LCMS method: 25.

### Example 44—Synthesis of N-[3-(3-chloro-4-cyano-phenoxy)-2,2,4,4-tetramethyl-cyclobutyl]-2-[4-[1-[3-[4-(1-hydroxy-1-methyl-ethyl)-2-(6-methyl-7-oxo-1H-pyrrolo[2,3-c]pyridin-4-yl)phenoxy]cyclopentyl]azetidin-3-yl]oxy-1-piperidyl]pyrimidine-5-carboxamide (I-266)

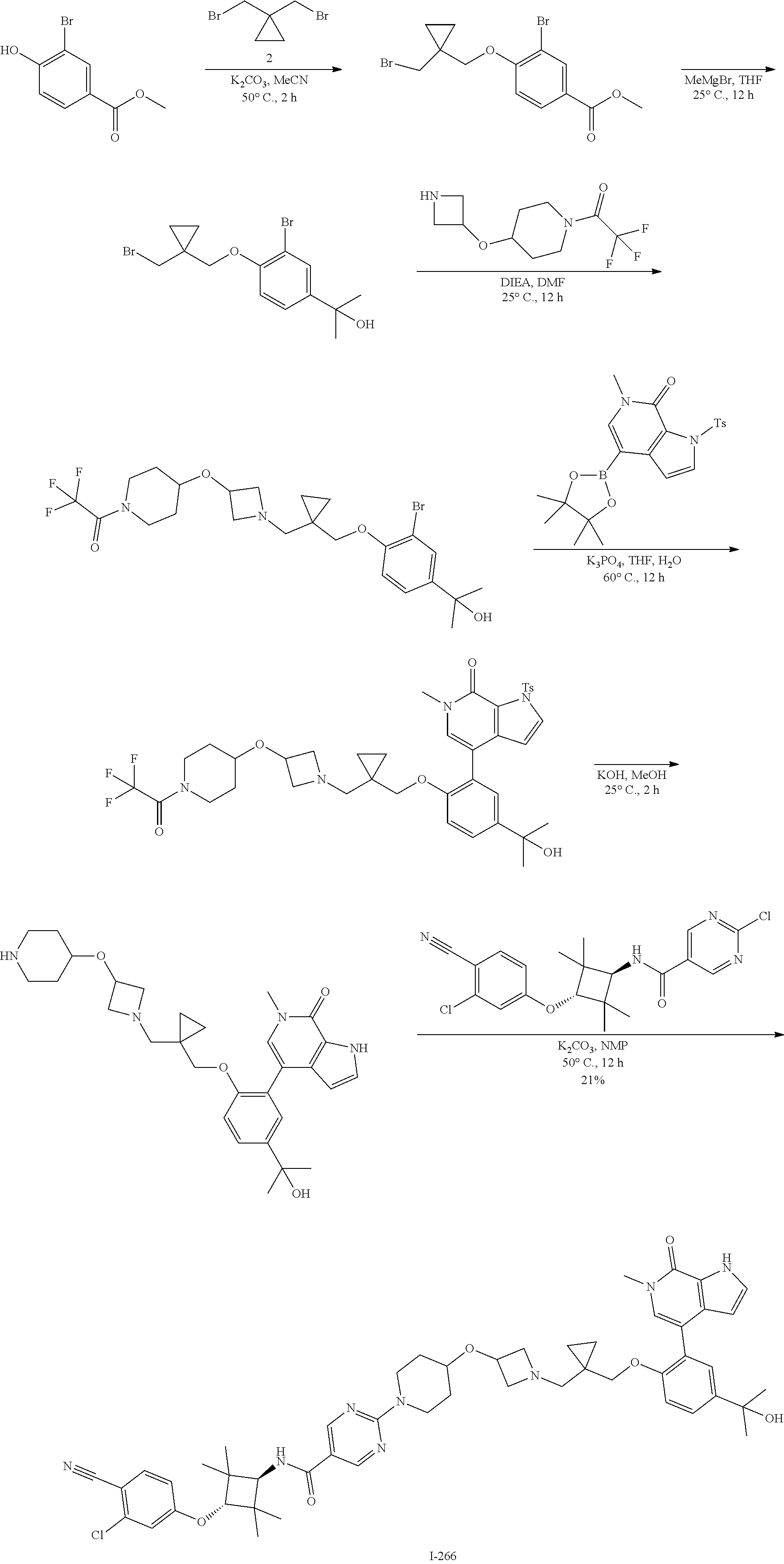

Step 1: Preparation of methyl 3-bromo-4-[[1-(bromomethyl)cyclopropyl]methoxy]benzoate. To a solution of 1,1-bis(bromomethyl)cyclopropane (8.88 g, 39 mmol, 3 equiv) and methyl 3-bromo-4-hydroxy-benzoate (3 g, 12 mmol, 1 equiv) in MeCN (30 mL) was added K₂CO₃ (5.38 g, 38 mmol, 3 equiv). The mixture was stirred at 80° C. for 12 h. The residue was diluted with H₂O 50 mL and extracted with EA (50 mL*3). The combined organic layers were washed with brine (50 mL*3), dried over Na₂SO₄, filtered and concentrated. The residue was purified by column chromatography (SiO₂, petroleum ether: ethyl acetate=50:1 to 5:1) to give methyl 3-bromo-4-[[1-(bromomethyl)cyclopropyl]methoxy]benzoate (2 g, 5 mmol, 41% yield).

Step 2: Preparation of 2-[3-bromo-4-[[1-(bromomethyl) cyclopropyl]methoxy]phenyl]propan-2-ol. To a solution of methyl 3-bromo-4-[[1-(bromomethyl) cyclopropyl]methoxy]benzoate (1 g, 2.6 mmol, 1 equiv) in THF (10 mL) was added MeMgBr (3 M, 8.8 mL, 10 equiv) at 0° C. The mixture was stirred at 25° C. for 12 h. The residue was diluted with H₂O 50 mL and extracted with EA (50 mL*3). The combined organic layers were washed with sat. NaCl (50 mL*3), dried over Na₂SO₄, filtered and concentrated. The residue was purified by column chromatography (SiO₂, petroleum ether: ethyl acetate=50:1 to 3:1) to give 2-[3-bromo-4-[[1-(bromomethyl) cyclopropyl]methoxy]phenyl]propan-2-ol (500 mg, 50% yield).

Step 3: Preparation of 1-[4-[1-[[1-[[2-bromo-4-(1-hydroxy-1-methyl-ethyl)phenoxy]methyl]cyclopropyl]methyl]azetidin-3-yl]oxy-1-piperidyl]-2,2,2-trifluoro-ethanone. To a solution of 2-[3-bromo-4-[[1-(bromomethyl)cyclopropyl]methoxy]phenyl]propan-2-ol (214 mg, 566 μmol, 1 equiv) and 1-[4-(azetidin-3-yloxy)-1-piperidyl]-2,2,2-trifluoro-ethanone (143 mg, 566.94 μmol, 1 equiv) in DMF (2 mL) was added DIEA (366 mg, 2.8 mmol, 5 equiv). The mixture was stirred at 25° C. for 12 h. The residue was filtered and concentrated. The residue was purified by prep-HPLC (column: Phenomenex luna C18 150 mm*25 mm*10 μm; mobile phase: [water (FA)-ACN]; B %: 18%-38%, 10 min). to give 1-[4-[1-[[1-[[2-bromo-4-(1-hydroxy-1-methyl-ethyl)phenoxy]methyl]cyclopropyl]methyl]azetidin-3-yl]oxy-1-piperidyl]-2,2,2-trifluoro-ethanone (167 mg, 53% yield).

Step 4: Preparation of 4-[5-(1-hydroxy-1-methyl-ethyl)-2-[[1-[[3-[[1-(2,2,2-trifluoroacetyl)-4-piperidyl]oxy]azetidin-1-yl]methyl]cyclopropyl]methoxy]phenyl]-6-methyl-1-(p-tolylsulfonyl)pyrrolo[2,3-c]pyridin-7-one. To a solution of 6-methyl-1-(p-tolylsulfonyl)-4-(4,4,5,5-tetramethyl-1,3,2-dioxaborolan-2-yl)pyrrolo[2,3-c]pyridin-7-one (145 mg, 340 μmol, 1.2 equiv) and 1-[4-[1-[[1-[[2-bromo-4-(1-hydroxy-1-methyl-ethyl)phenoxy]methyl]cyclopropyl]methyl]azetidin-3-yl]oxy-1-piperidyl]-2,2,2-trifluoro-ethanone (156 mg, 283 umol, 1 equiv) in THF (5 mL) and H₂O(1 mL) was added K₃PO₄ (180 mg, 851 mol, 3 equiv) and SPhos Pd G3 (22 mg, 28 mol, 0.1 equiv). The mixture was stirred at 60° C. for 12 h. The mixture was diluted with H₂O 20 mL and extracted with EA (20 mL). The combined organic layers were washed with sat. NaCl (20 mL*3), dried over Na₂SO₄, and concentrated. The residue was purified by prep-HPLC (column: Phenomenex Luna C18 150 mm*25 mm*10 4 μm; mobile phase: [water (FA)-ACN]; B %: 21%-51%, 9 min) to give the desired product 4-[5-(1-hydroxy-1-methyl-ethyl)-2-[[1-[[3-[[1-(2,2,2-trifluoroacetyl)-4-piperidyl]oxy]azetidin-1-yl]methyl]cyclopropyl]methoxy]phenyl]-6-methyl-1-(p-tolylsulfonyl)pyrrolo[2,3-c]pyridin-7-one (167 mg, 76% yield).

Step 5: Preparation of 4-[5-(1-hydroxy-1-methyl-ethyl)-2-[[1-[[3-[[1-(2,2,2-trifluoroacetyl)-4-piperidyl]oxy]azetidin-1-yl]methyl]cyclopropyl]methoxy]phenyl]-6-methyl-1-(p-tolylsulfonyl)pyrrolo[2,3-c]pyridin-7-one. To a solution of 6-methyl-1-(p-tolylsulfonyl)-4-(4,4,5,5-tetramethyl-1,3,2-dioxaborolan-2-yl)pyrrolo[2,3-c]pyridin-7-one (145 mg, 340 μmol, 1.2 equiv) and 1-[4-[1-[[1-[[2-bromo-4-(1-hydroxy-1-methyl-ethyl)phenoxy]methyl]cyclopropyl]methyl]azetidin-3-yl]oxy-1-piperidyl]-2,2,2-trifluoro-ethanone (156 mg, 284 μmol, 1 equiv) in THF (5 mL) and H₂O (1 mL) was added K₃PO₄ (181 mg, 851 μmol, 3 equiv) and SPhos Pd G3 (22 mg, 28 μmol, 0.1 equiv). The mixture was stirred at 60° C. for 12 h. The mixture was diluted with H₂O (20 mL) and extracted with EA (20 mL). The combined organic layers were washed with sat. NaCl (20 mL*3), dried over Na₂SO₄, concentrated. The residue was purified by prep-HPLC (column: Phenomenex Luna C18 150 mm*25 mm*10 μm; mobile phase: [water (FA)-ACN]; B %: 21%-51%, 9 min) to give 4-[5-(1-hydroxy-1-methyl-ethyl)-2-[[1-[[3-[[1-(2,2,2-trifluoroacetyl)-4-piperidyl]oxy]azetidin-1-yl]methyl]cyclopropyl]methoxy]phenyl]-6-methyl-1-(p-tolylsulfonyl)pyrrolo[2,3-c]pyridin-7-one (167 mg, 76% yield).

Step 6: Preparation of N-[3-(3-chloro-4-cyano-phenoxy)-2,2,4,4-tetramethyl-cyclobutyl]-2-[4-[1-[3-[4-(1-hydroxy-1-methyl-ethyl)-2-(6-methyl-7-oxo-1H-pyrrolo[2,3-c]pyridin-4-yl)phenoxy]cyclopentyl]azetidin-3-yl]oxy-1-piperidyl]pyrimidine-5-carboxamide (1-266). To a solution of 2-chloro-N-[3-(3-chloro-4-cyano-phenoxy)-2,2,4,4-tetramethyl-cyclobutyl]pyrimidine-5-carboxamide (62 mg, 149 mol, 1 equiv) and 4-[5-(1-hydroxy-1-methyl-ethyl)-2-[[1-[[3-(4-piperidyloxy)azetidin-1-yl]methyl]cyclopropyl]methoxy]phenyl]-6-methyl-1H-pyrrolo[2,3-c]pyridin-7-one (78 mg, 149 μmol, 1 equiv) in NMP (2 mL) was added K₂CO₃ (103 mg, 749 μmol, 5 equiv). The mixture was stirred at 50° C. for 1 h. The residue was filtered and concentrated. The residue was purified by prep-HPLC (column: Phenomenex luna C18 150 mm*25 mm*10 μm; mobile phase: [water(FA)-ACN]; B %: 26%-56%, 9 min) to give N-[3-(3-chloro-4-cyano-phenoxy)-2,2,4,4-tetramethyl-cyclobutyl]-2-[4-[1-[3-[4-(1-hydroxy-1-methyl-ethyl)-2-(6-methyl-7-oxo-1H-pyrrolo[2,3-c]pyridin-4-yl)phenoxy]cyclopentyl]azetidin-3-yl]oxy-1-piperidyl]pyrimidine-5-carboxamide (28 mg, 210% yield). ¹H NMR: ¹H NMR (400 MHz, DMSO-d₆) δ 11.96 (s, 1H), 8.78-8.71 (m, 2H), 7.93-7.86 (m, 1H), 7.74-7.68 (m, 1H), 7.45 (d, J=2.4 Hz, 1H), 7.38-7.31 (m, 1H), 7.28-7.24 (m, 1H), 7.23-7.17 (m, 2H), 7.04-6.98 (m, 1H), 6.96 (d, J=8.8 Hz, 1H), 6.16-6.10 (m, 1H), 5.00-4.81 (m, 1H), 4.33-4.22 (m, 3H), 4.16-4.11 (m, 1H), 4.06-4.00 (m, 1H), 3.79-3.74 (m, 2H), 3.56 (s, 3H), 3.53-3.47 (m, 3H), 2.75-2.64 (m, 2H), 2.29 (s, 2H), 1.88-1.71 (m, 2H), 1.52-1.38 (m, 7H), 1.37-1.23 (m, 4H), 1.21 (s, 6H), 1.11 (s, 6H), 0.40-0.21 (m, 4H). LC-MS: MS (ES⁺): RT=1.920 min, m/z=903.5[M+1]; LCMS method: 25.

### Example 45—Synthesis of N-[3-(3-chloro-4-cyano-phenoxy)-2,2,4,4-tetramethyl-cyclobutyl]-2-[7-[2-[3-[4-ethylsulfonyl-2-(6-methyl-7-oxo-1H-pyrrolo[2,3-c]pyridin-4-yl)phenoxy]cyclobutyl]ethyl]-2,7-diazaspiro[4.4]nonan-2-yl]pyrimidine-5-carboxamide (1-270)

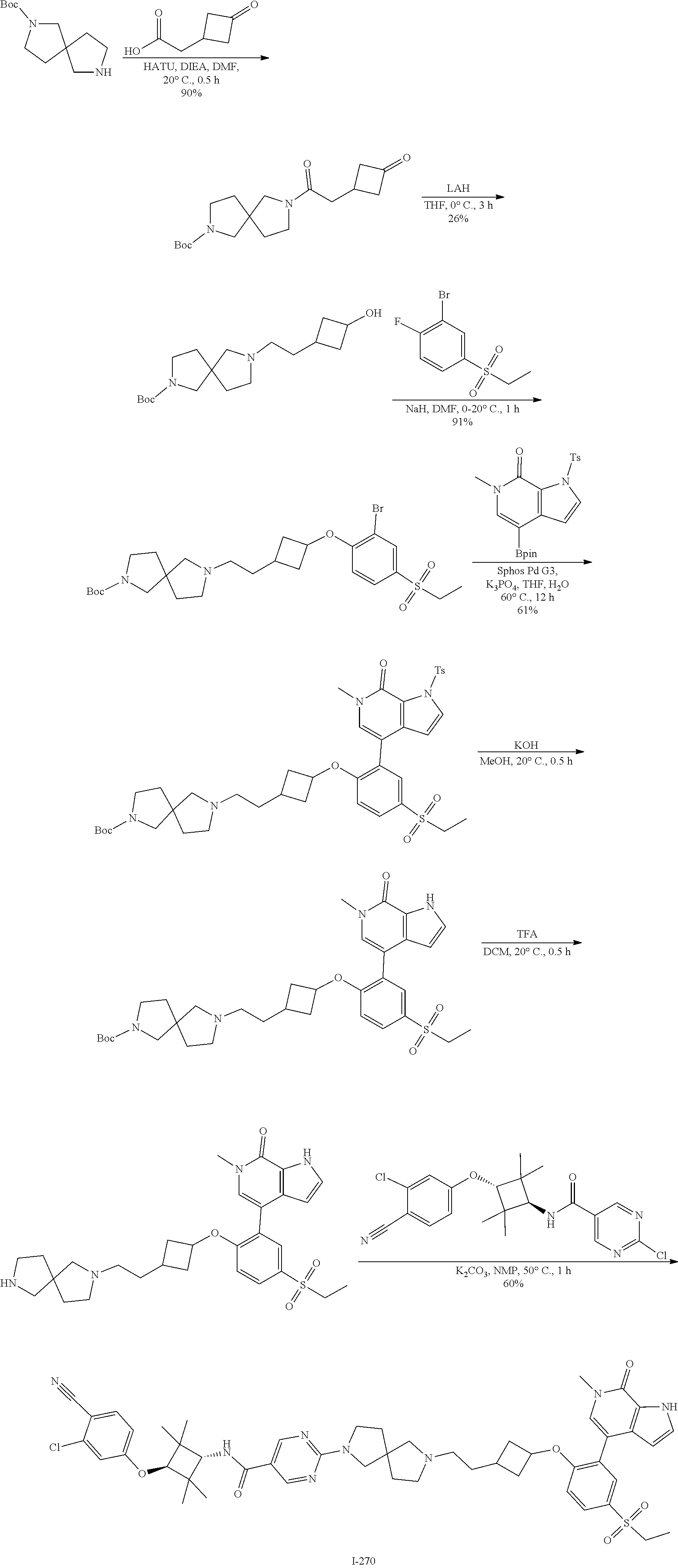

Step 1: Preparation of tert-butyl 7-[2-(3-oxocyclobutyl)acetyl]-2,7-diazaspiro[4.4]nonane-2-carboxylate. To a solution of tert-butyl 2,7-diazaspiro[4.4]nonane-2-carboxylate (3 g, 13.26 mmol, 1.0 equiv), DIEA (5.14 g, 40 mmol, 3.0 equiv) in DMF (20 mL) was added HATU (6.05 g, 15.9 mmol, 1.2 equiv) and 2-(3-oxocyclobutyl)acetic acid (1.7 g, 13 mmol, 1.0 equiv). The mixture was stirred at 20° C. for 0.5 h. The residue was purified by prep-HPLC (column: Phenomenex luna C18 250 mm*80 mm*10 μm; mobile phase: [water(FA)-ACN]; B %: 35%-65%, 20 min) to give tert-butyl 7-[2-(3-oxocyclobutyl)acetyl]-2,7-diazaspiro[4.4]nonane-2-carboxylate (4 g, 90% yield) as a yellow gum.

Step 2: Preparation of tert-butyl 7-[2-(3-hydroxycyclobutyl)ethyl]-2,7-diazaspiro[4.4]nonane-2-carboxylate. To a solution of tert-butyl 7-[2-(3-oxocyclobutyl)acetyl]-2,7-diazaspiro[4.4]nonane-2-carboxylate (1 g, 2.97 mmol, 1.0 equiv) in THF (25 mL) was added LAH (500 mg, 13.17 mmol, 4.4 equiv) at 0° C. The mixture was stirred at 0° C. for 3 h. The reaction mixture was quenched with aq. NH₄Cl (30 mL). The mixture was extracted with DCM (50 mL*3), the combined organic layer was washed with brine (30 mL), dried over anhydrous Na₂SO₄, and the mixture was filtered and the filtrate was concentrated in vacuo. The residue was purified by prep-HPLC (column: Phenomenex Luna C18 150 mm*25 mm*10 μm; mobile phase: [water(FA)-ACN]; B %: 1%-30%, 10 min) to give tert-butyl 7-[2-(3-hydroxycyclobutyl)ethyl]-2,7-diazaspiro[4.4]nonane-2-carboxylate (250 mg, 26% yield) as a yellow oil.

Step 3: Preparation of tert-butyl 7-[2-[3-(2-bromo-4-ethylsulfonyl-phenoxy)cyclobutyl]ethyl]-2,7-diazaspiro[4.4]nonane-2-carboxylate. To a solution of tert-butyl 7-[2-(3-hydroxycyclobutyl)ethyl]-2,7-diazaspiro[4.4]nonane-2-carboxylate (250 mg, 770 μmol, 1.0 equiv) in THF (10 mL) was added NaH (46 mg, 1.16 mmol, 60% purity, 1.5 equiv) and 2-bromo-4-ethylsulfonyl-1-fluoro-benzene (205 mg, 770 μmol, 1.0 equiv). The mixture was stirred at 50° C. for 1 h. The residue was purified by silica gel chromatography (SiO₂, petroleum ether: ethyl acetate=10:1 to 0:1) to give tert-butyl 7-[2-[3-(2-bromo-4-ethylsulfonyl-phenoxy)cyclobutyl]ethyl]-2,7-diazaspiro[4.4]nonane-2-carboxylate (400 mg, 91% yield) as a yellow gum.

Step 4: Preparation of tert-butyl 7-[2-[3-[4-ethylsulfonyl-2-[6-methyl-7-oxo-1-(p-tolylsulfonyl)pyrrolo[2,3-c]pyridin-4-yl]phenoxy]cyclobutyl]ethyl]-2,7-diazaspiro[4.4]nonane-2-carboxylate. To a solution of 6-methyl-1-(p-tolylsulfonyl)-4-(4,4,5,5-tetramethyl-1,3,2-dioxaborolan-2-yl)pyrrolo[2,3-c]pyridin-7-one (180 mg, 420 μmol, 1.2 equiv) and tert-butyl 7-[2-[3-(2-bromo-4-ethylsulfonyl-phenoxy)cyclobutyl]ethyl]-2,7-diazaspiro[4.4]nonane-2-carboxylate (200 mg, 350 μmol, 1.0 equiv) in THF (10 mL) was added H₂O (1 mL), SPhos Pd G3 (27 mg, 35 μmol, 0.1 equiv) and K₃PO₄ (148 mg, 699 mol, 2.0 equiv). The mixture was stirred at 60° C. for 12 h. The residue was purified by prep-HPLC (SiO₂, DCM: MeOH=10:1) to give tert-butyl 7-[2-[3-[4-ethylsulfonyl-2-[6-methyl-7-oxo-1-(p-tolylsulfonyl)pyrrolo[2,3-c]pyridin-4-yl]phenoxy]cyclobutyl]ethyl]-2,7-diazaspiro[4.4]nonane-2-carboxylate (170 mg, 61% yield) as a yellow gum.

Step 5: Preparation of tert-butyl 7-[2-[3-[4-ethylsulfonyl-2-(6-methyl-7-oxo-1H-pyrrolo[2,3-c]pyridin-4-yl)phenoxy]cyclobutyl]ethyl]-2,7-diazaspiro[4.4]nonane-2-carboxylate. To a solution of tert-butyl 7-[2-[3-[4-ethylsulfonyl-2-[6-methyl-7-oxo-1-(p-tolylsulfonyl)pyrrolo[2,3-c]pyridin-4-yl]phenoxy]cyclobutyl]ethyl]-2,7-diazaspiro[4.4]nonane-2-carboxylate (200 mg, 252 μmol, 1.0 equiv) in MeOH (10 mL) was added KOH (283 mg, 5.0 mmol, 20.0 equiv). The mixture was stirred at 20° C. for 0.5 h. The mixture was extracted with DCM (30 mL*3), the combined organic was washed with brine (80 mL), dried over anhydrous Na₂SO₄, and the mixture was filtered and the filtrate was concentrated to give tert-butyl 7-[2-[3-[4-ethylsulfonyl-2-(6-methyl-7-oxo-1H-pyrrolo[2,3-c]pyridin-4-yl) phenoxy]cyclobutyl]ethyl]-2,7-diazaspiro[4.4]nonane-2-carboxylate (160 mg) as a yellow gum.

Step 6: Preparation of 4-[2-[3-[2-(2,7-diazaspiro[4.4]nonan-2-yl)ethyl]cyclobutoxy]-5-ethylsulfonyl-phenyl]-6-methyl-1H-pyrrolo[2,3-c]pyridin-7-one. To a solution of tert-butyl 7-[2-[3-[4-ethylsulfonyl-2-(6-methyl-7-oxo-1H-pyrrolo[2,3-c]pyridin-4-yl)phenoxy]cyclobutyl]ethyl]-2,7-diazaspiro[4.4]nonane-2-carboxylate (200 mg, 313 μmol, 1.0 equiv) in DCM (1 mL) was added TFA (0.3 mL). The mixture was stirred at 20° C. for 0.5 h. The mixture was concentrated to give 4-[2-[3-[2-(2,7-diazaspiro[4.4]nonan-2-yl)ethyl]cyclobutoxy]-5-ethylsulfonyl-phenyl]-6-methyl-1H-pyrrolo[2,3-c]pyridin-7-one (180 mg, TFA salt) as a yellow gum.

Step 7: Preparation of N-[3-(3-chloro-4-cyano-phenoxy)-2,2,4,4-tetramethyl-cyclobutyl]-2-[7-[2-[3-[4-ethylsulfonyl-2-(6-methyl-7-oxo-1H-pyrrolo[2,3-c]pyridin-4-yl)phenoxy]cyclobutyl]ethyl]-2,7-diazaspiro[4.4]nonan-2-yl]pyrimidine-5-carboxamide (I-270). To a solution of 4-[2-[3-[2-(2,7-diazaspiro[4.4]nonan-2-yl)ethyl]cyclobutoxy]-5-ethylsulfonyl-phenyl]-6-methyl-1H-pyrrol [2,3-c]pyridin-7-one (100 mg, 153 μmol, 1.0 equiv, TFA salt) in NMP (5 mL) was added K₂CO₃ (63 mg, 459 mol, 3.0 equiv) and 2-chloro-N-[3-(3-chloro-4-cyano-phenoxy)-2,2,4,4-tetramethyl-cyclobutyl]pyrimidine-5-carboxamide (64 mg, 153 mol, 1.0 equiv). The mixture was stirred at 50° C. for 1 h. The residue was purified by prep-HPLC (column: YMC Triart C18 150 mm*25 mm * Sum; mobile phase: [water(FA)-ACN]; B %: 30%-60%, 8.5 min) to give the desired product N-[3-(3-chloro-4-cyano-phenoxy)-2,2,4,4-tetramethyl-cyclobutyl]-2-[7-[2-[3-[4-ethylsulfonyl-2-(6-methyl-7-oxo-1H-pyrrolo[2,3-c]pyridin-4-yl)phenoxy]cyclobutyl]ethyl]-2,7-diazaspiro[4.4]nonan-2-yl]pyrimidine-5-carboxamide (85 mg, 60% yield) as a white solid. ¹H NMR (400 MHz, DMSO-d₆): δ 12.10-11.99 (m, 1H), 8.89-8.63 (m, 2H), 7.95-7.88 (m, 1H), 7.85-7.76 (m, 2H), 7.71 (d, J=9.2 Hz, 1H), 7.36-7.28 (m, 2H), 7.25-7.08 (m, 2H), 7.05-6.98 (m, 1H), 6.06 (s, 1H), 4.71 (t, J=7.2 Hz, 1H), 4.30 (s, 1H), 4.08-4.02 (m, 1H), 3.60-3.56 (m, 5H), 3.49-3.46 (m, 2H), 3.32-3.26 (m, 2H), 2.82-2.60 (m, 6H), 2.27-2.06 (m, 2H), 2.04-1.90 (m, 3H), 1.87-1.78 (m, 2H), 1.76-1.53 (m, 4H), 1.25-1.19 (m, 6H), 1.17-1.08 (m, 9H). LC-MS: MS (ES⁺): RT=1.854 min, m/z=921.4 [M+H⁺]; LCMS method: 25

### Example 46—Synthesis of N-[3-(3-chloro-4-cyano-phenoxy)-2,2,4,4-tetramethyl-cyclobutyl]-2-[4-[3-[4-(1-hydroxy-1-methyl-ethyl)-2-(6-methyl-7-oxo-1H-pyrrolo[2,3-c]pyridin-4-yl)phenoxy]cyclobutoxy]-1-piperidyl]pyrimidine-5-carboxamide (I-274)

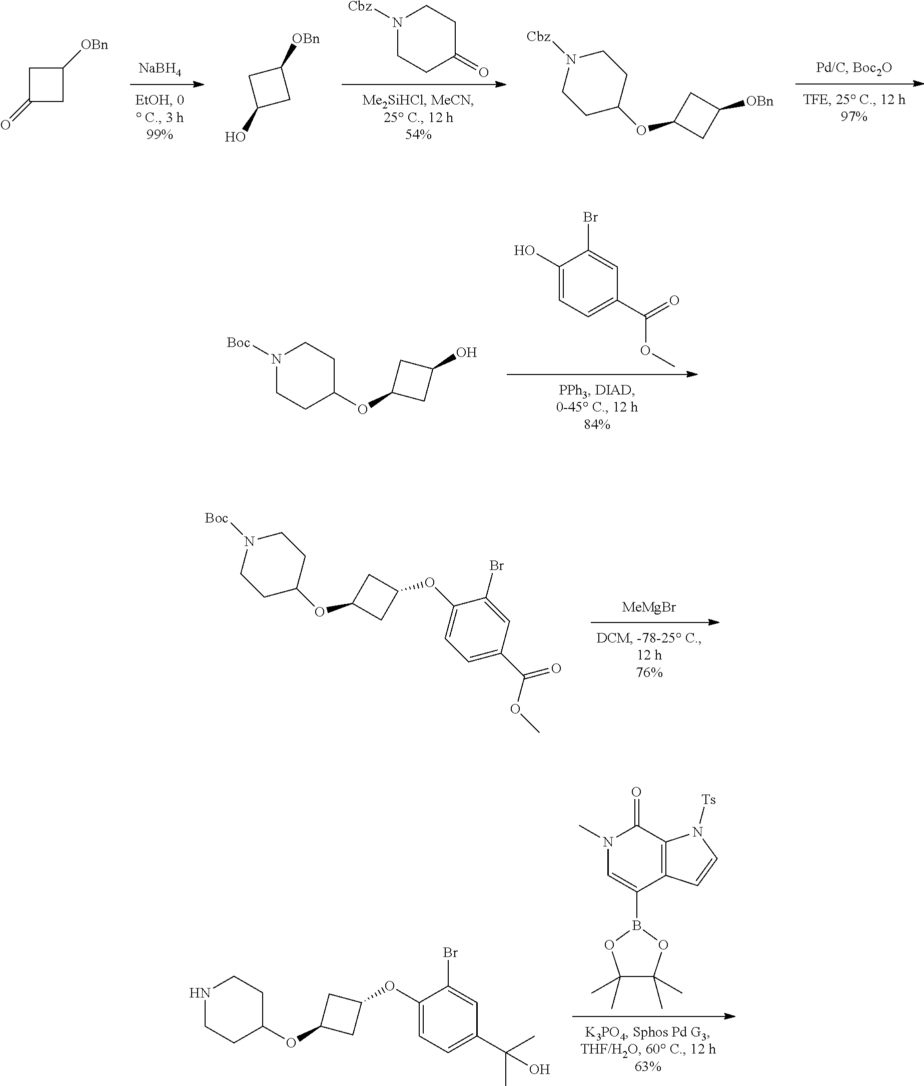

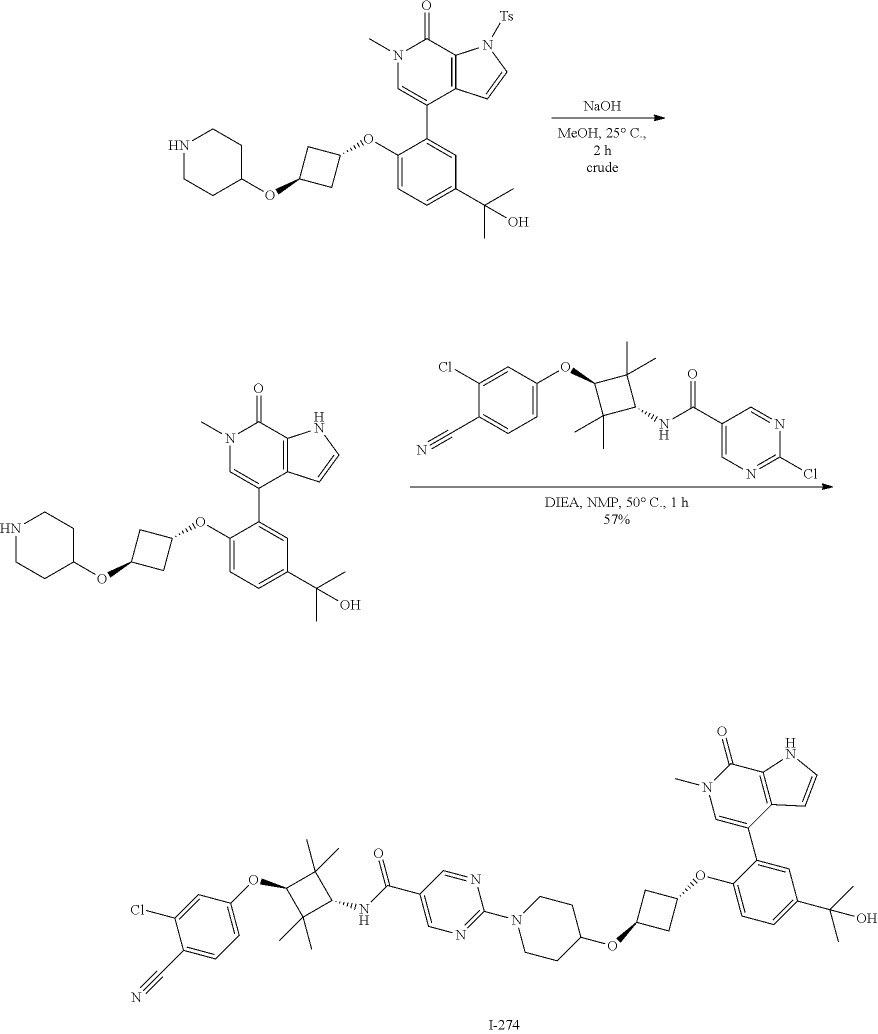 [SMILES: Cc1ccc(S(=O)(=O)n2ccc3c(-c4cc(C(C)(C)O)ccc4O[C@H]4C[C@H](OC5CCNCC5)C4)cn(C)c(=O)c32)cc1 ; "NaOH" ; Cn1cc(-c2cc(C(C)(C)O)ccc2O[C@H]2C[C@H](OC3CCNCC3)C2)c2cc[nH]c2c1=O ; CC1(C)[C@H](NC(=O)c2cnc(Cl)nc2)C(C)(C)[C@H]1Oc1ccc(C#N)c(Cl)c1 ; Cn1cc(-c2cc(C(C)(C)O)ccc2O[C@H]2C[C@H](OC3CCN(c4ncc(C(=O)N[C@H]5C(C)(C)[C@H](Oc6ccc(C#N)c(Cl)c6)C5(C)C)cn4)CC3)C2)c2cc[nH]c2c1=O]

Step 1: Preparation of 3-benzyloxycyclobutanol. To a solution of 3-benzyloxycyclobutanone (6.0 g, 34 mmol, 1.0 equiv) in EtOH (100 mL) was added NaBH₄ (1.46 g, 38.6 mmol, 1.1 equiv). The mixture was stirred at 0° C. for 3 h. The reaction mixture was quenched with saturated aqueous NH₄Cl (100 mL) solution at 0° C. dropwise. And the resulting mixture was extracted with ethyl acetate (50 mL×3). The combined organic layers were dried over anhydrous Na₂SO₄, filtered and concentrated in vacuum. The residue was purified by column chromatography (SiO₂, petroleum ether. ethyl acetate=20.1 to 3:1). The compound 3-benzyloxycyclobutanol (6.0 g, 34 mmol, 99% yield) was obtained as a white solid.

Step 2: Preparation of benzyl 4-(3-benzyloxycyclobutoxy)piperidine-1-carboxylate. To a solution of 3-benzyloxycyclobutanol (5.0 g, 28 mmol, 1.0 equiv), benzyl 4-oxopiperidine-1-carboxylate (6.54 g, 28.1 mmol, 5.59 mL, 1.0 equiv) in MeCN (50 mL) was added chloro(dimethyl)silane (2.92 g, 30.9 mmnol, 1.1 equiv). The mixture was stirred at 25° C. for 12 h. To the reaction mixture was added water (50 mL) and the mixture was extracted with ethyl acetate (50 mL×3). The combined organic phase was washed with brine (50 mL), dried over anhydrous Na₂SO₄, filtered and concentrated in vacuo. The residue was purified by column chromatography (SiO₂, petroleum ether: ethyl acetate=20:1 to 3:1). The compound benzyl 4-(3-benzyloxycyclobutoxy)piperidine-1-carboxylate (6.0 g, 15 mmol, 54% yield) was obtained as a white solid.

Step 3: Preparation of tert-butyl 4-(3-hydroxycyclobutoxy)piperidine-1-carboxylate. To a solution of benzyl 4-(3-benzyloxycyclobutoxy)piperidine-1-carboxylate (3.0 g, 7.6 mmol, 1.0 equiv) in TFE (30 mL) was added Pd/C (2.0 g, 6.1 mmol, 10% purity, 0.8 equiv) and Boc₂O (2.48 g, 11.4 mmol, 2.61 mL, 1.5 equiv) under N₂. The suspension was degassed under vacuum and purged with H₂ several times. The mixture was stirred under H₂ (15 psi) at 25° C. for 12 h. The reaction mixture was filtered and the filtrate was concentrated. The residue was purified by column chromatography (SiO₂, petroleum ether: ethyl acetate=20:1 to 3:1). The compound tert-butyl 4-(3-hydroxycyclobutoxy)piperidine-1-carboxylate (2.0 g, 7.4 mmol, 97% yield) was obtained as a white solid.

Step 4: Preparation of tert-butyl 4-13-(2-bromo-4-methoxycarbonyl-phenoxy) cyclobutoxy]piperidine-1-carboxylate. To a solution of tert-butyl 4-[3-hydroxycyclobutoxy) piperidine-1-carboxylate (2.0 g, 7.4 mmol, 1.0 equiv), methyl 3-bromo-4-hydroxy-benzoate (1.7 g, 7.4 mmol, 1.0 equiv) in THF (20 mL) was added PPh₃ (5.8 g, 22 mmol, 3.0 equiv) and DIAD (4.47 g, 22.1 mmol, 4.30 mL, 3.0 equiv). The mixture was stirred at 0-45° C. for 2 h. The reaction mixture was concentrated in vacuo to give the crude product. The crude product was purified by preparative HPLC (column: Phenomenex luna C18 150 mm*40 mm*m;mobile phase: [water(FA)-ACN]; B %: 67%-97%, 10 min) to yield the compound tert-butyl 4-[3-(2-bromo-4-methoxycarbonyl-phenoxy)cyclobutoxy]piperidine-1-carboxylate (3.0 g, 6.2 mmol, 84% yield) as a white solid.

Step 5: Preparation of 2-[3-bromo-4-[3-(4-piperidyloxy)cyclobutoxy]phenyl]propan-2-ol. To a solution of tert-butyl 4-[3-(2-bromo-4-methoxycarbonyl-phenoxy)cyclobutoxy]piperidine-1-carboxylate (1.0 g, 2.1 mmol, 1.0 equiv) in DCM (10 mL) was added MeMgBr (3.0 M, 10 mL, 15.0 equiv). The mixture was stirred at -78-25° C. for 12 h. To the reaction mixture was added water (30 mL) and the mixture was extracted with dichloromethane (30 mL×3). The combined organic phase was washed with brine (30 mL), dried over anhydrous Na₂SO₄, filtered and concentrated in vacuo. The crude product was purified by preparative HPLC (column: Phenomenex luna C18 150 mm*40 mm*15 μm; mobile phase: [water(FA)-ACN]; B %: 6%-36%, 10 min). The compound 2-[3-bromo-4-[3-(4-piperidyloxy)cyclobutoxy]phenyl]propan-2-ol (600 mg, 1.56 mmol, 76% yield) was obtained as a white solid.

Step 6: Preparation of 4-[5-(1-hydroxy-1-methyl-ethyl)-2-[3-(4-piperidyloxy) cyclobutoxy]phenyl]-6-methyl-1-(p-tolylsulfonyl)pyrrolo[2,3-c]pyridin-7-one. A mixture of 2-[3-bromo-4-[3-(4-piperidyloxy)cyclobutoxy]phenyl]propan-2-ol (300 mg, 781 mol, 1.0 equiv), 6-methyl-1-(p-tolylsulfonyl)-4-(4,4,5,5-tetramethyl-1,3,2-dioxaborolan-2-yl) pyrrolo[2,3-c]pyridin-7-one (435 mg, 1.01 mmol, 1.3 equiv), SPhos Pd G3 (61 mg, 78 4 mol, 0.1 equiv), K₃PO₄ (1.5 M, 1.6 mL, 3.0 equiv) in THF (10 mL) was degassed and purged with N₂ three times, and then the mixture was stirred at 60° C. for 12 h under an N₂ atmosphere. The reaction was filtered and the filtrate was concentrated in vacuo to give the crude product. The crude product was purified by preparative HPLC (column: Phenomenex luna C18 150 mm*25 mm*10 μm; mobile phase: [water(FA)-ACN]; B %: 15%-45%, 10 min). The compound 4-[5-(1-hydroxy-1-methyl-ethyl)-2-[3-(4-piperidyloxy)cyclobutoxy]phenyl]-6-methyl-1-(p-tolylsulfonyl)pyrrolo[2,3-c]pyridin-7-one (300 mg, 495 μmol, 63% yield) was obtained as a white solid.

Step 7: Preparation of 4-[5-(1-hydroxy-1-methyl-ethyl)-2-[3-(4-piperidyloxy) cyclobutoxy]phenyl]-6-methyl-1H-pyrrolo[2,3-c]pyridin-7-one. To a solution of 4-[5-(1-hydroxy-1-methyl-ethyl)-2-[3-(4-piperidyloxy)cyclobutoxy]phenyl]-6-methyl-1-(p-tolylsulfonyl)pyrrolo[2,3-c]pyridin-7-one (150 mg, 248 mol, 1.0 equiv) in MeOH (3 mL) was added NaOH (1.0 M, 3.7 mL, 15.0 equiv). The mixture was stirred at 25° C. for 2 h. The reaction was filtered and the filtrate was concentrated in vacuo to give the crude product. The compound 4-[5-(1-hydroxy-1-methyl-ethyl)-2-[3-(4-piperidyloxy)cyclobutoxy]phenyl]-6-methyl-1H-pyrrolo[2,3-c]pyridin-7-one (110 mg, crude) was obtained as a white solid.

Step 8: Preparation of N-[3-(3-chloro-4-cyano-phenoxy)-2,2,4,4-tetramethyl-cyclobutyl]-2-[4-[3-[4-(1-hydroxy-1-methyl-ethyl)-2-(6-methyl-7-oxo-1H-pyrrolo[2,3-c]pyridin-4-yl)phenoxy]cyclobutoxy]-1-piperidyl]pyrimidine-5-carboxamide (I-274). To a solution of 4-[5-(1-hydroxy-1-methyl-ethyl)-2-[3-(4-piperidyloxy)cyclobutoxy]phenyl]-6-methyl-1H-pyrrolo[2,3-c]pyridin-7-one (100 mg, 221 μmol, 1.0 equiv) in NMP (2 mL) was added DIEA (143 mg, 1.11 mmol, 193 μL, 5.0 equiv) and 2-chloro-N-[3-(3-chloro-4-cyano-phenoxy)-2,2,4,4-tetramethyl-cyclobutyl]pyrimidine-5-carboxamide (93 mg, 0.22 mmol, 1.0 equiv). The mixture was stirred at 50° C. for 1 h. The reaction was filtered and the filtrate was concentrated in vacuo to give the crude product. The crude product was purified by preparative HPLC (column: Phenomenex luna C18 150*25 mm* 10 um;mobile phase: [water(FA)-ACN];B %: 58%-88%, 10 min) to yield. Compound N-[3-(3-chloro-4-cyano-phenoxy)-2,2,4,4-tetramethyl-cyclobutyl]-2-[4-[3-[4-(1-hydroxy-1-methyl-ethyl)-2-(6-methyl-7-oxo-1H-pyrrolo[2,3-c]pyridin-4-yl)phenoxy]cyclobutoxy]-1-piperidyl]pyrimidine-5-carboxamide (106 mg, 125 μmol, 57% yield, 99% purity) was obtained as a white solid. ¹H NMR (400 MHz, DMSO-d₆) δ 11.96 (s, 1H), 8.75 (s, 2H), 7.90 (d, J=8.8 Hz, 1H), 7.72 (d, J=9.2 Hz, 1H), 7.44 (d, J=2.4 Hz, 1H), 7.36 (s, 1H), 7.28 (t, J=2.8 Hz, 1H), 7.24-7.16 (m, 2H), 7.00 (s, 1H), 6.78 (d, J=8.8 Hz, 1H), 6.12 (t, J=2.0 Hz, 1H), 4.93 (s, 1H), 4.78 (s, 1H), 4.35-4.22 (m, 4H), 4.03 (d, J=9.2 Hz, 1H), 3.62-3.49 (m, 4H), 3.42-3.37 (m, 2H), 2.35-2.29 (m, 2H), 2.27-2.19 (m, 2H), 1.86-1.77 (m, 2H), 1.43 (s, 6H), 1.39-1.30 (m, 2H), 1.21 (s, 6H), 1.11 (s, 6H). LC-MS: MS (ES⁺): RT=2.73 min, m/z=834.4 [M+H⁺]; LCMS method: 25.

### Example 47—Synthesis of N-[3-(3-chloro-4-cyano-phenoxy)-2,2,4,4-tetramethyl-cyclobutyl]-2-[4-[2-[6-[4-(1-hydroxy-1-methyl-ethyl)-2-(6-methyl-7-oxo-1H-pyrrolo[2,3-c]pyridin-4-yl)phenoxy]-2-azaspiro[3.3]heptan-2-yl]ethyl]-1-piperidyl]pyrimidine-5-carboxamide (I-281)

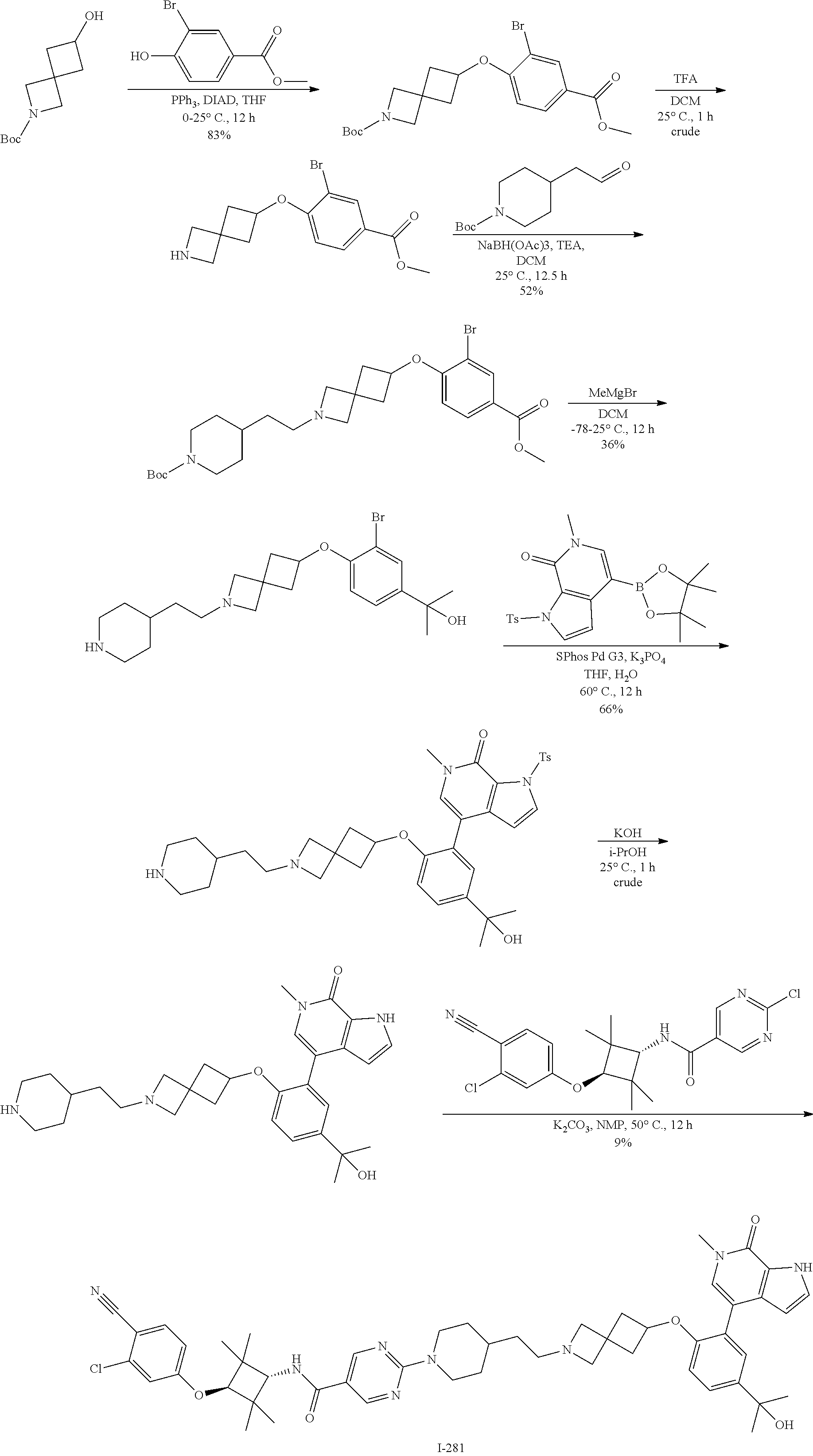

Step 1: Preparation of tert-butyl 6-(2-bromo-4-methoxycarbonyl-phenoxy)-2-azaspiro[3.3]heptane-2-carboxylate. To a solution of tert-butyl 6-hydroxy-2-azaspiro[3.3]heptane-2-carboxylate (3.0 g, 14 mmol, 1.0 equiv), methyl 3-bromo-4-hydroxy-benzoate (3.25 g, 14.0 mmol, 1.0 equiv) and PPh₃ (11.07 g, 42.20 mmol, 3.0 equiv) in THF (60 mL) was added DIAD (8.53 g, 42.2 mmol, 3.0 equiv) at 0° C. under an N₂ atmosphere. The mixture was stirred at 25° C. for 12 h. The reaction mixture was poured into ice water (100 mL), and then extracted with EA (100 mL×3). The combined organic layers were washed with brine (50 mL×3), dried over Na₂SO₄, filtered and concentrated under reduced pressure to give a residue. The residue was purified by prep-HPLC (FA condition; column: Phenomenex luna C18 250 mm*100 mm*15 μm; mobile phase: [water(FA)-ACN]; B %: 55%-75%, 25 min). The desired compound tert-butyl 6-(2-bromo-4-methoxycarbonyl-phenoxy)-2-azaspiro[3.3]heptane-2-carboxylate (5.0 g, 12 mmol, 83% yield) was obtained as white solid.

Step 2: Preparation of methyl 4-(2-azaspiro[3.3]heptan-6-yloxy)-3-bromo-benzoate. To a solution of tert-butyl 6-(2-bromo-4-methoxycarbonyl-phenoxy)-2-azaspiro[3.3]heptane-2-carboxylate (2.50 g, 5.86 mmol, 1.0 equiv) in DCM (25 mL) was added TFA (19.25 g, 168.8 mmol, 28.8 equiv). The mixture was stirred at 25° C. for 1 h. The reaction mixture was concentrated under reduced pressure to give a residue. The residue was used in the next step without further purification. The desired compound methyl 4-(2-azaspiro[3.3]heptan-6-yloxy)-3-bromo-benzoate (1.91 g, crude) was obtained as yellow oil.

Step 3: Preparation of tert-butyl 4-[2-[6-(2-bromo-4-methoxycarbonyl-phenoxy)-2-azaspiro[3.3]heptan-2-yl]ethyl]piperidine-1-carboxylate. To a solution of methyl 4-(2-azaspiro[3.3]heptan-6-yloxy)-3-bromo-benzoate (1.91 g, 5.86 mmol, 1.0 equiv) in DCM (33 mL) was added TEA (5.93 g, 58.6 mmol, 10.0 equiv) and tert-butyl 4-(2-oxoethyl)piperidine-1-carboxylate (1.33 g, 5.86 mmol, 1.0 equiv), the mixture was stirred for 0.5 h, and then NaBH(OAc)₃ (4.96 g, 23.4 mmol, 4.0 equiv) was added. The mixture was stirred at 25° C. for 12 h. The reaction mixture was poured into water (50 mL), and then extracted with DCM (100 mL×3). The combined organic layers were washed with brine (50 mL×2), dried over Na₂SO₄, filtered and concentrated under reduced pressure to give a residue. The residue was purified by prep-HPLC (FA condition; column: Phenomenex luna C18 150 mm*40 mm*15 μm; mobile phase: [water(FA)-ACN]; B %: 20%-50%, 15 min). The desired compound tert-butyl 4-[2-[6-(2-bromo-4-methoxycarbonyl-phenoxy)-2-azaspiro[3.3]heptan-2-yl]ethyl]piperidine-1-carboxylate (1.65 g, 3.07 mmol, 52% yield) was obtained as white gum.

Step 4: Preparation of 2-[3-bromo-4-[[2-[2-(4-piperidyl)ethyl]-2-azaspiro[3.3]heptan-6-yl]oxy]phenyl]propan-2-ol. A mixture of tert-butyl 4-[2-[6-(2-bromo-4-methoxycarbonyl-phenoxy)-2-azaspiro[3.3]heptan-2-yl]ethyl]piperidine-1-carboxylate (517 mg, 962 mol, 1.0 equiv) in DCM (4.8 mL) was degassed and purged with N₂ three times, and then MeMgBr (3.0 M, 4.8 mL, 15.0 equiv) was added to the solution dropwise at −78° C. The mixture was stirred at 25° C. for 12 h under an N₂ atmosphere. The reaction mixture was quenched by addition of NH₄Cl aq. (10 mL), and then extracted with EA (10 mL×3). The combined organic layers were washed with brine (10 mL×3), dried over Na₂SO₄, filtered and concentrated under reduced pressure to give a residue. The residue was purified by prep-HPLC (FA condition; column: Phenomenex luna C18 150 mm*25 mm*10 μm; mobile phase: [water(FA)-ACN]; B %: 1%-30%, 10 min). The desired compound 2-[3-bromo-4-[[2-[2-(4-piperidyl)ethyl]-2-azaspiro[3.3]heptan-6-yl]oxy]phenyl]propan-2-ol (150 mg, 343 μmol, 36% yield) was obtained as off-white solid.

Step 5: Preparation of 4-[5-(1-hydroxy-1-methyl-ethyl)-2-[[2-[2-(4-piperidyl)ethyl]-2-azaspiro[3.3]heptan-6-yl]oxy]phenyl]-6-methyl-1-(p-tolylsulfonyl) pyrrolo[2,3-c]pyridin-7-one. To a solution of 6-methyl-1-(p-tolylsulfonyl)-4-(4,4,5,5-tetramethyl-1,3,2-dioxaborolan-2-yl)pyrrolo[2,3-c]pyridin-7-one (118 mg, 274 μmol. 1.0 equiv) and 2-[3-bromo-4-[[2-[2-(4-piperidyl)ethyl]-2-azaspiro[3.3]heptan-6-yl]oxy]phenyl]propan-2-ol (120 mg, 274 μmol, 1.0 equiv) in THF (4 mL) and H₂O (0.8 mL) was added KPO4 (175 mg, 823 4 mol, 3.0 equiv), and then SPhos Pd G3 (43 mg, 55 mol, 0.2 equiv) was added and purged with N₂ three times. The mixture was stirred at 60° C. for 12 h. The reaction mixture was concentrated under reduced pressure to remove THF. The residue was diluted with ACN (40 mL), and then filtered and concentrated under reduced pressure to give a residue. The residue was purified by prep-HPLC (FA condition; column: Phenomenex luna C18 150 mm*25 mm*10 μm; mobile phase: [water(FA)-ACN]; B %: 7%-37%, 10 min). The desired compound 4-[5-(1-hydroxy-1-methyl-ethyl)-2-[[2-[2-(4-piperidyl)ethyl]-2-azaspiro[3.3]heptan-6-yl]oxy]phenyl]-6-methyl-1-(p-tolylsulfonyl)pyrrolo[2,3-c]498yridine-7-one (120 mg, 182 mol, 66% yield) was obtained as off-white solid.

Step 6: Preparation of 4-[5-(1-hydroxy-1-methyl-ethyl)-2-[[2-[2-(4-piperidyl)ethyl]-2-azaspiro[3.3]heptan-6-yl]oxy]phenyl]-6-methyl-1H-pyrrolo[2,3-c]pyridine-7-one. To a solution of 4-[5-(1-hydroxy-1-methyl-ethyl)-2-[[2-[2-(4-piperidyl)ethyl]-2-azaspiro[3.3]heptan-6-yl]oxy]phenyl]-6-methyl-1-(p-tolylsulfonyl) pyrrolo[2,3-c]pyridine-7-one (120 mg, 182 μmol, 1.0 equiv) in i-PrOH (1.5 mL) was added KOH (153 mg, 2.73 mmol, 15.0 equiv). The mixture was stirred at 25° C. for 1 h. The reaction mixture was quenched by addition of water (20 mL), and then extracted with EA (50 mL×3). The combined organic layers were washed with water (50 mL×3), dried over Na₂SO₄, filtered and concentrated under reduced pressure to give a residue. The reaction mixture was used in the next step without further purification. The desired compound 4-[5-(1-hydroxy-1-methyl-ethyl)-2-[[2-[2-(4-piperidyl)ethyl]-2-azaspiro [3.3]heptan-6-yl]oxy]phenyl]-6-methyl-1H-pyrrolo[2,3-c]pyridin-7-one (90 mg, crude) was obtained as an off-white solid.

Step 7: Preparation of N-[3-(3-chloro-4-cyano-phenoxy)-2,2,4,4-tetramethyl-cyclobutyl]-2-[4-[2-[6-[4-(1-hydroxy-1-methyl-ethyl)-2-(6-methyl-7-oxo-1H-pyrrolo[2,3-c]pyridin-4-yl)phenoxy]-2-azaspiro[3.3]heptan-2-yl]ethyl]-1-piperidyl]pyrimidine-5-carboxamide (1-281). To a solution of 2-chloro-N-[3-(3-chloro-4-cyano-phenoxy)-2,2,4,4-tetramethyl-cyclobutyl]pyrimidine-5-carboxamide (82 mg, 0.20 mmol, 1.1 equiv) and 4-[5-(1-hydroxy-1-methyl-ethyl)-2-[[2-[2-(4-piperidyl)ethyl]-2-azaspiro[3.3]heptan-6-yl]oxy]phenyl]-6-methyl-1H-pyrrolo[2,3-c]pyridin-7-one (90 mg, 0.18 mmol, 1.0 equiv) in NMP (2 mL) was added K₂CO₃ (246 mg, 1.78 mmol, 10.0 equiv). The reaction mixture was stirred at 50° C. for 12 h. The reaction mixture was filtered. The residue was purified by prep-HPLC (FA condition; column: Phenomenex luna C18 150 mm*25 mm*10 μm; mobile phase: [water(FA)-ACN]; B %: 27%-57%, 10 min). The desired compound N-[3-(3-chloro-4-cyano-phenoxy)-2,2,4,4-tetramethyl-cyclobutyl]-2-[4-[2-[6-[4-(1-hydroxy-1-methyl-ethyl)-2-(6-methyl-7-oxo-1H-pyrrolo[2,3-c]pyridin-4-yl)phenoxy]-2-azaspiro[3.3]heptan-2-yl]ethyl]-1-piperidyl]pyrimidine-5-carboxamide (15 mg, 16 mol, 9% yield) was obtained as white solid.

¹H NMR (400 MHz, DMSO-d₆) δ=11.96-11.91 (m, 1H), 8.73 (s, 2H), 7.92-7.87 (m, 1H), 7.73-7.67 (m, 1H), 7.44-7.40 (m, 1H), 7.36-7.32 (m, 1H), 7.27-7.24 (m, 1H), 7.23-7.20 (m, 1H), 7.18-7.14 (m, 1H), 7.03-6.98 (m, 1H), 6.84-6.78 (m, 1H), 6.11-6.06 (m, 1H), 4.96-4.87 (m, 1H), 4.75-4.66 (m, 2H), 4.60-4.51 (m, 1H), 4.30-4.25 (m, 1H), 4.06-3.99 (m, 1H), 3.55 (s, 3H), 3.15 (s, 2H), 3.04 (s, 2H), 2.97-2.87 (m, 2H), 2.69-2.65 (m, 1H), 2.40-2.30 (m, 4H), 2.04-1.96 (m, 2H), 1.75-1.67 (m, 2H), 1.64-1.53 (m, 1H), 1.44-1.40 (m, 6H), 1.21 (s, 6H), 1.18-1.14 (m, 2H), 1.11 (s, 6H), 1.02-0.98 (m, 1H). LC-MS: MS (ES^{T}): RT=1.955 min, m/z=887.4 [M+H⁺]; LCMS method: 25.

### Example 48—Synthesis of N-[3-(3-chloro-4-cyano-phenoxy)-2,2,4,4-tetramethyl-cyclobutyl]-2-[4-[1-[3-[4-(1-hydroxy-1-methyl-ethyl)-2-(6-methyl-7-oxo-1H-pyrrolo[2,3-c]pyridin-4-yl)phenoxy]-2,2-dimethyl-propyl]azetidin-3-yl]oxy-1-piperidyl]pyrimidine-5-carboxamide (I-282)

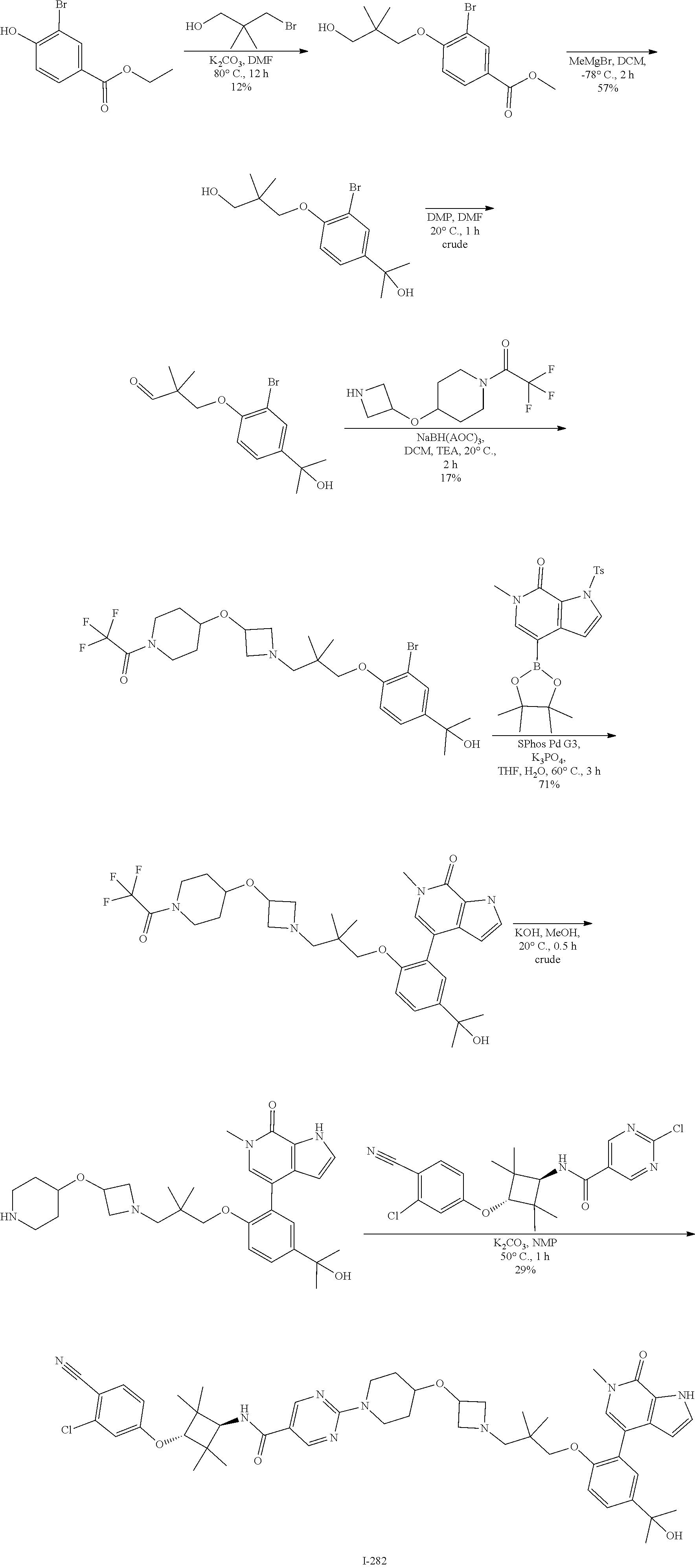

Step 1: Preparation of methyl 3-bromo-4-(3-hydroxy-2,2-dimethyl-propoxy)benzoate. To a solution of methyl 3-bromo-4-hydroxy-benzoate (2.5 g, 10.82 mmol, 1 equiv) in DMF (20 mL) was added K₂CO₃ (2.99 g, 21.64 mmol, 2 equiv) and 3-bromo-2,2-dimethyl-propan-1-ol (2.35 g, 14.07 mmol, 1.69 mL, 1.3 equiv). The mixture was stirred at 80° C. for 12 h. The residue was purified by prep-HPLC (column: Phenomenex luna C18 150 mm*40 mm*15 μm; mobile phase: [water(FA)-ACN]; B %: 43%-73%, 10 min) to give methyl 3-bromo-4-(3-hydroxy-2,2-dimethyl-propoxy)benzoate (1.7 g, 12% yield).

Step 2: Preparation of 3-[2-bromo-4-(1-hydroxy-1-methyl-ethyl)phenoxy]-2,2-dimethyl-propan-1-ol. To a solution of methyl 3-bromo-4-(3-hydroxy-2,2-dimethyl-propoxy)benzoate (700 mg, 2.21 mmol, 1 equiv) in DCM (20 mL) was added MeMgBr (3 M, 11 mL, 15 equiv). The mixture was stirred at −78° C. for 2 h. The reaction mixture was quenched by addition NH₄Cl 20 ml at 0° C., and then diluted with DCM 10 mL and extracted with DCM 30 ml. The combined organic layers were washed with H₂O 20 mL, dried over Na₂SO₄, filtered and concentrated under reduced pressure to give a residue. The residue was purified by prep-HPLC (column: Phenomenex luna C18 150 mm*40 mm*15 um; mobile phase: [water(FA)-ACN]; B %: 32%-62%, 10 min) to give 3-[2-bromo-4-(1-hydroxy-1-methyl-ethyl)phenoxy]-2,2-dimethyl-propan-1-ol (400 mg, 57% yield) was a colorless oil.

Step 3: Preparation of 3-[2-bromo-4-(1-hydroxy-1-methyl-ethyl)phenoxy]-2,2-dimethyl-propanal. To a solution of 3-[2-bromo-4-(1-hydroxy-1-methyl-ethyl)phenoxy]-2,2-dimethyl-propan-1-ol (400 mg, 1.26 mmol, 1 equiv) in DMF (5 mL) was added DMP (1.07 g, 2.52 mmol, 2 equiv). The mixture was stirred at 20° C. for 1 h. The reaction mixture was filtered and concentrated under reduced pressure to give crude product 3-[2-bromo-4-(1-hydroxy-1-methyl-ethyl)phenoxy]-2,2-dimethyl-propanal (395 mg) was a yellow oil.

Step 4: Preparation of 1-[4-[1-[3-[2-bromo-4-(1-hydroxy-1-methyl-ethyl)phenoxy]-2,2-dimethyl-propyl]azetidin-3-yl]oxy-1-piperidyl]-2,2,2-trifluoro-ethanone. To a solution of 3-[2-bromo-4-(1-hydroxy-1-methyl-ethyl)phenoxy]-2,2-dimethyl-propanal (395 mg, 1.25 mmol, 1 equiv) in DCM (35 mL) was added TEA (1.27 g, 12.53 mmol, 10 equiv), 1-[4-(azetidin-3-yloxy)-1-piperidyl]-2,2,2-trifluoro-ethanone (459 mg, 1.25 mmol, 1 equiv, TFA salt) and NaBH(OAc)₃ (2.66 g, 12.53 mmol, 10 equiv). The mixture was stirred at 20° C. for 2 h. The residue was purified by prep-HPLC (column: Phenomenex luna C18 150 mm*40 mm*15 um; mobile phase: [water(FA)-ACN]; B %: 14%-44%, 15 min) to give 1-[4-[1-[3-[2-bromo-4-(1-hydroxy-1-methyl-ethyl)phenoxy]-2,2-dimethyl-propyl]azetidin-3-yl]oxy-1-piperidyl]-2,2,2-trifluoro-ethanone (120 mg, 17% yield).

Step 5: Preparation of 4-[2-[2,2-dimethyl-3-[3-[[1-(2,2,2-trifluoroacetyl)-4-piperidyl]oxy]azetidin-1-yl]propoxy]-5-(1-hydroxy-1-methyl-ethyl)phenyl]-6-methyl-1-(p-tolylsulfonyl)pyrrolo[2,3-c]pyridin-7-one. To a solution of 1-[4-[1-[3-[2-bromo-4-(1-hydroxy-1-methyl-ethyl)phenoxy]-2,2-dimethyl-propyl]azetidin-3-yl]oxy-1-piperidyl]-2,2,2-trifluoro-ethanone (120 mg, 218 mol, 1 equiv) in THF (2.5 mL) was added SPhos Pd G3 (17 mg, 22 mol, 0.1 equiv), 6-methyl-1-(p-tolylsulfonyl)-4-(4,4,5,5-tetramethyl-1,3,2-dioxaborolan-2-yl)pyrrolo[2,3-c]pyridin-7-one (103 mg, 239 mol, 1.1 equiv), H₂O (0.5 mL) and K₃PO₄ (139 mg, 653 mol, 3 equiv). The mixture was stirred at 60° C. for 3 h. The residue was purified by prep-HPLC (column: Phenomenex luna C18 150 mm*25 mm*10 4 μm; mobile phase: [water(FA)-ACN]; B %: 26%-56%, 10 min) to give 4-[2-[2,2-dimethyl-3-[3-[[1-(2,2,2-trifluoroacetyl)-4-piperidyl]oxy]azetidin-1-yl]propoxy]-5-(1-hydroxy-1-methyl-ethyl)phenyl]-6-methyl-1-(p-tolylsulfonyl)pyrrolo[2,3-c]pyridin-7-one (120 mg, 71% yield) as a yellow oil.

Step 6: Preparation of 4-[2-[2,2-dimethyl-3-[3-(4-piperidyloxy)azetidin-1-yl]propoxy]-5-(1-hydroxy-1-methyl-ethyl)phenyl]-6-methyl-1H-pyrrolo[2,3-c]pyridin-7-one. To a solution of 4-[2-[2,2-dimethyl-3-[3-[[1-(2,2,2-trifluoroacetyl)-4-piperidyl]oxy]azetidin-1-yl]propoxy]-5-(1-hydroxy-1-methyl-ethyl)phenyl]-6-methyl-1-(p-tolylsulfonyl)pyrrolo[2,3-c]pyridin-7-one (120 mg, 155 mol, 1 equiv) in MeOH (1.5 mL) was added KOH (2 M, 1.80 mL, 23 equiv). The mixture was stirred at 20° C. for 0.5 h. The reaction mixture was filtered and concentrated under reduced pressure to give the crude product 4-[2-[2,2-dimethyl-3-[3-(4-piperidyloxy)azetidin-1-yl]propoxy]-5-(1-hydroxy-1-methyl-ethyl)phenyl]-6-methyl-1H-pyrrolo[2,3-c]pyridin-7-one (80 mg) as a yellow oil.

Step 7: Preparation of N-[3-(3-chloro-4-cyano-phenoxy)-2,2,4,4-tetramethyl-cyclobutyl]-2-[4-[1-[3-[4-(1-hydroxy-1-methyl-ethyl)-2-(6-methyl-7-oxo-1H-pyrrolo[2,3-c]pyridin-4-yl)phenoxy]-2,2-dimethyl-propyl]azetidin-3-yl]oxy-1-piperidyl]pyrimidine-5-carboxamide (I-282). To a solution of 4-[2-[2,2-dimethyl-3-[3-(4-piperidyloxy)azetidin-1-yl]propoxy]-5-(1-hydroxy-1-methyl-ethyl)phenyl]-6-methyl-H-pyrrolo[2,3-c]pyridin-7-one (80 mg, 153 4 mol, 1 equiv) in NMP (2 mL) was added K₂CO₃ (64 mg, 459 μmol, 3 equiv) and2-chloro-N-[3-(3-chloro-4-cyano-phenoxy)-2,2,4,4-tetramethyl-cyclobutyl]pyrimidine-5-carboxamide (64 mg, 153 4 mol, 1 equiv). The mixture was stirred at 50° C. for 1 h. The residue was purified by prep-HPLC (column: Phenomenex luna C18 150 mm*25 mm*10 μm; mobile phase: [water(FA)-ACN]; B %: 27%-57%, 10 min) to give N-[3-(3-chloro-4-cyano-phenoxy)-2,2,4,4-tetramethyl-cyclobutyl]-2-[4-[1-[3-[4-(1-hydroxy-1-methyl-ethyl)-2-(6-methyl-7-oxo-1H-pyrrolo[2,3-c]pyridin-4-yl)phenoxy]-2,2-dimethyl-propyl]azetidin-3-yl]oxy-1-piperidyl]pyrimidine-5-carboxamide (40 mg, 29% yield). 1HNMR (400 MHz, MeOD) δ 8.74 (s, 2H), 7.72 (d, J=8.68 Hz, 1H), 7.52 (d, J=2.44 Hz, 1H), 7.45-7.48 (m, 1H), 7.32 (d, J=2.80 Hz, 1H), 7.20 (s, 1H), 7.13 (d, J=2.32 Hz, 1H), 7.03 (d, J=8.56 Hz, 1H), 6.96-6.99 (m, 1H), 6.24 (d, J=2.80 Hz, 1H), 4.30-4.36 (m, 2H), 4.27 (s, 1H), 4.09-4.18 (m, 2H), 3.71 (s, 3H), 3.68 (s, 2H), 3.52-3.62 (m, 3H), 3.45-3.49 (m, 2H), 2.91-2.95 (m, 2H), 2.22-2.39 (m, 2H), 1.74-1.87 (m, 2H), 1.56 (s, 6H), 1.39-1.48 (m, 2H), 1.28 (s, 6H), 1.21 (s, 6H), 0.82 (s, 6H). LC-MS: MS (ES⁺): RT=1.967 min, m/z=905.4 [M+H⁺]; LCMS method: 25.

### Example 49—Synthesis of N-[3-(3-chloro-4-cyano-phenoxy)-2,2,4,4-tetramethyl-cyclobutyl]-2-[4-[2-[6-[4-(1-hydroxy-1-methyl-ethyl)-2-(6-methyl-7-oxo-1H-pyrrolo[2,3-c]pyridin-4-yl)phenoxy]-3-pyridyl]ethyl]-1-piperidyl]pyrimidine-5-carboxamide (I-287)

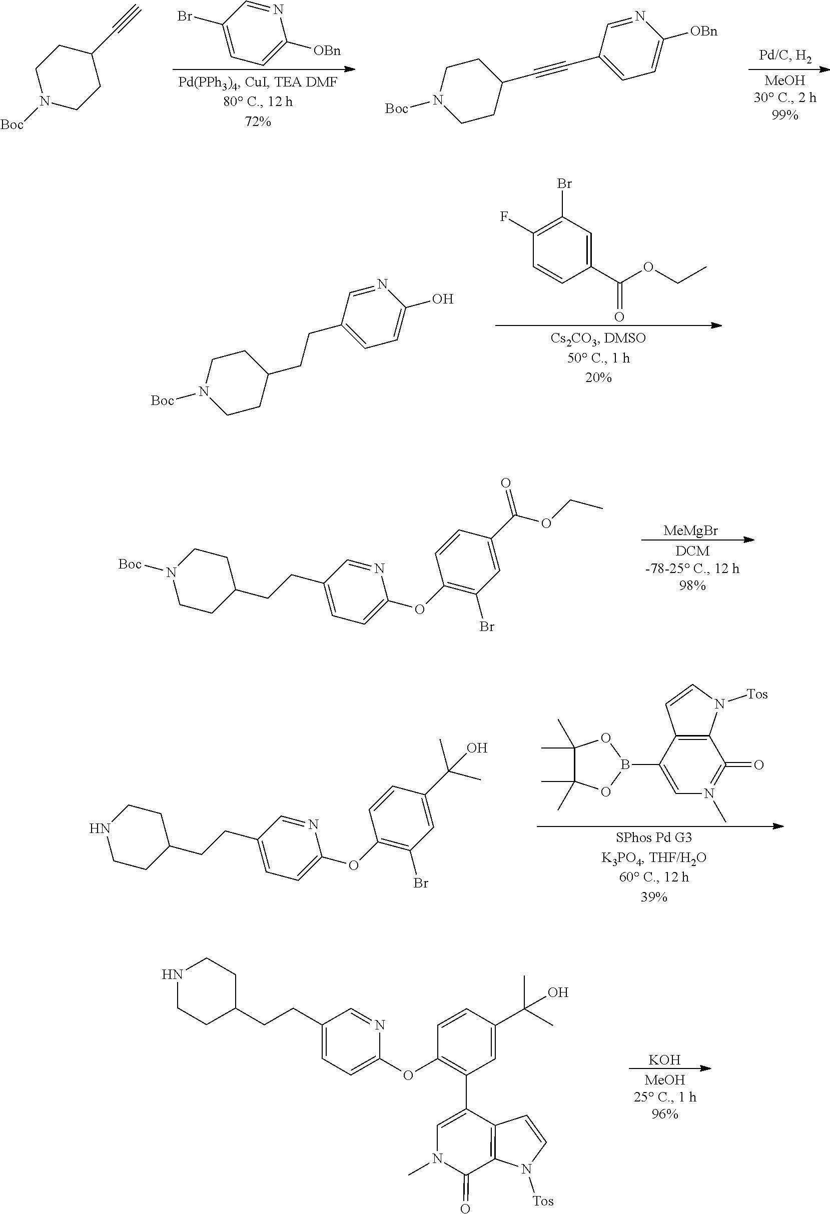

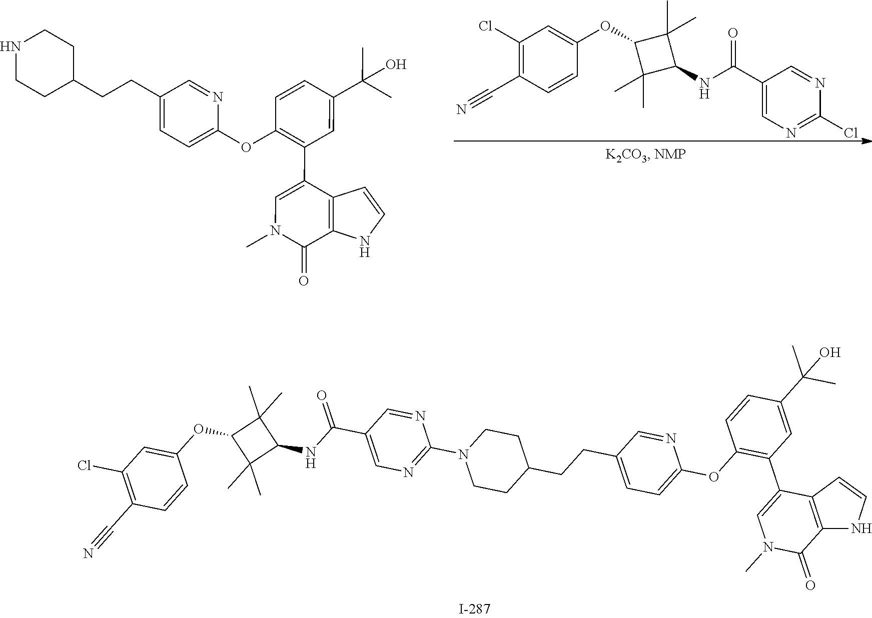 [SMILES: Cn1cc(-c2cc(C(C)(C)O)ccc2Oc2ccc(CCC3CCNCC3)cn2)c2cc[nH]c2c1=O ; CC1(C)[C@H](NC(=O)c2cnc(Cl)nc2)C(C)(C)[C@H]1Oc1ccc(C#N)c(Cl)c1 ; Cn1cc(-c2cc(C(C)(C)O)ccc2Oc2ccc(CCC3CCN(c4ncc(C(=O)N[C@H]5C(C)(C)[C@H](Oc6ccc(C#N)c(Cl)c6)C5(C)C)cn4)CC3)cn2)c2cc[nH]c2c1=O]

Step 1: Preparation of tert-butyl 4-[2-(6-benzyloxy-3-pyridyl) ethynyl]piperidine-1-carboxylate. A mixture of tert-butyl 4-ethynylpiperidine-1-carboxylate (5.5 g, 26 mmol, 1.0 equiv), 2-benzyloxy-5-bromo-pyridine (7 g, 26 mmol, 1.0 equiv), CuI (1.0 g, 5.30 mmol, 0.2 equiv), Pd(PPh₃)₄(6.1 g, 5.30 mmol, 0.2 equiv) in DMF (90 mL) and TEA (30 mL) was degassed and purged with N₂ three times, and then the mixture was stirred at 80° C. for 12 h under an N₂ atmosphere. The reaction mixture was partitioned between H₂O (50 mL) and EA (200 mL). The organic phase was separated, washed with H₂O 30 (30 mL*3), dried over Na₂SO₄, filtered and concentrated. The residue was purified by column chromatography (SiO₂, petroleum ether: ethyl acetate=20:1 to 15:1) to give tert-butyl 4-[2-(6-benzyloxy-3-pyridyl) ethynyl]piperidine-1-carboxylate (7.5 g, 72% yield).

Step 2: Preparation of tert-butyl 4-(2-(6-hydroxypyridin-3-yl)ethyl)piperidine-1-carboxylate. To a solution of tert-butyl 4-[2-(6-benzyloxy-3-pyridyl)ethynyl]piperidine-1-carboxylate (4 g, 10 mmol, 1.0 equiv) in MeOH (30 mL) was added Pd/C (2 g, 10% purity) under an N₂ atmosphere. The mixture was stirred under H₂ (15 psi.) at 30° C. for 2 h. The mixture was filtered and concentrated. The residue was used directly for the next step.

Step 3: Preparation of tert-butyl 4-[2-[6-(2-bromo-4-ethoxycarbonyl-phenoxy)-3-pyridyl]ethyl]piperidine-1-carboxylate. To a solution of tert-butyl 4-[2-(6-hydroxy-3-pyridyl)ethyl]piperidine-1-carboxylate (2.8 g, 9.1 mmol, 1.0 equiv) and ethyl 3-bromo-4-fluoro-benzoate (2.2 g, 9.1 mmol, 1.0 equiv) in NMP (30 mL) was added Cs₂CO₃ (8.9 g, 27 mmol, 3.0 equiv). The mixture was stirred at 50° C. for 1 h. The reaction mixture was partitioned between H₂O 50 mL and EA 200 mL. The organic phase was separated and washed with H₂O 30 mL (30 mL*3), dried over Na₂SO₄, filtered and concentrated. The residue was purified by prep-HPLC (column: Waters Xbridge C18 150 mm*50 mm*10 μm; mobile phase: [water (NH₄HCO₃)-ACN]; B %: 64%-94%, 10 min) to give tert-butyl 4-[2-[6-(2-bromo-4-ethoxycarbonyl-phenoxy)-3-pyridyl]ethyl]piperidine-1-carboxylate (1 g, 20% yield).

Step 4: Preparation of 2-[3-bromo-4-[[5-[2-(4-piperidyl)ethyl]-2-pyridyl]oxy]phenyl]propan-2-ol. To a solution of tert-butyl 4-[2-[6-(2-bromo-4-ethoxycarbonyl-phenoxy)-3-pyridyl]ethyl]piperidine-1-carboxylate (900 mg, 1.69 mmol, 1.0 equiv) in DCM (16 mL) was added MeMgBr (3 M, 8.4 mL, 15 equiv) at −78° C. The mixture was stirred at 25° C. for 12 h and concentrated. The residue was purified by prep-HPLC (column: Phenomenex luna C18 150 mm*40 mm*15 um; mobile phase: [water(TFA)-ACN]; B %: 15%-45%, 10 min) to give 2-[3-bromo-4-[[5-[2-(4-piperidyl)ethyl]-2-pyridyl]oxy]phenyl]propan-2-ol (700 mg, 98% yield).

Step 5: Preparation of 4-[5-(1-hydroxy-1-methyl-ethyl)-2-[[5-[2-(4-piperidyl)ethyl]-2-pyridyl]oxy]phenyl]-6-methyl-1-(p-tolylsulfonyl)pyrrolo[2,3-c]pyridin-7-one. A mixture of 2-[3-bromo-4-[[5-[2-(4-piperidyl)ethyl]-2-pyridyl]oxy]phenyl]propan-2-ol (50 mg, 119 μmol, 1.0 equiv), 6-methyl-1-(p-tolylsulfonyl)-4-(4,4,5,5-tetramethyl-1,3,2-dioxaborolan-2-yl)pyrrolo[2,3-c]pyridin-7-one (56 mg, 131 mol, 1.1 equiv), SPhos Pd G3 (18 mg, 23 μmol, 0.2 equiv), K₃PO₄ (75 mg, 357 μmol, 3.0 equiv) in THF (5 mL) and H₂O (1 mL) was degassed and purged with N₂ three times, and then the mixture was stirred at 60° C. for 12 h under an N₂ atmosphere. The reaction mixture was partitioned between H₂O (50 mL) and EA (100 mL). The organic phase was separated and washed with H₂O (30 mL*3), dried over Na₂SO₄, filtered and concentrated to give 4-[5-(1-hydroxy-1-methyl-ethyl)-2-[[5-[2-(4-piperidyl)ethyl]-2-pyridyl]oxy]phenyl]-6-methyl-1-(p-tolylsulfonyl)pyrrolo[2,3-c]pyridin-7-one (30 mg, 39% yield).

Step 6: Preparation of 4-[5-(1-hydroxy-1-methyl-ethyl)-2-[[5-[2-(4-piperidyl)ethyl]-2-pyridyl]oxylphenyl]-6-methyl-1H-pyrrolo[2,3-c]pyridin-7-one. To a solution of 4-[5-(1-hydroxy-1-methyl-ethyl)-2-[[5-[2-(4-piperidyl)ethyl]-2-pyridyl]oxy]phenyl]-6-methyl-1-(p-tolylsulfonyl)pyrrolo[2,3-c]pyridin-7-one (30 mg, 46 μmol, 1.0 equiv) in MeOH (2 mL) was added KOH (39 mg, 702 mol, 15 equiv). The mixture was stirred at 25° C. for 1 h. The pH of the solution was adjusted to 5-6 with aqueous CH₃COOH, then the combined organic layer was concentrated. The residue was used for next step directly to give 4-[5-(1-hydroxy-1-methyl-ethyl)-2-[[5-[2-(4-piperidyl)ethyl]-2-pyridyl]oxy]phenyl]-6-methyl-1H-pyrrolo[2,3-c]pyridin-7-one (22 mg, 96% yield).

Step 7: Preparation of N-[3-(3-chloro-4-cyano-phenoxy)-2,2,4,4-tetramethyl-cyclobutyl]-2-[4-[2-[6-[4-(1-hydroxy-1-methyl-ethyl)-2-(6-methyl-7-oxo-1H-pyrrolo[2,3-c]pyridin-4-yl)phenoxy]-3-pyridyl]ethyl]-1-piperidyl]pyrimidine-5-carboxamide (I-287). To a solution of 4-[5-(1-hydroxy-1-methyl-ethyl)-2-[[5-[2-(4-piperidyl)ethyl]-2-pyridyl]oxy]phenyl]-6-methyl-¹H-pyrrolo[2,3-c]pyridin-7-one (110 mg, 226.6 μmol, 1.0 equiv) in NMP (2 mL) was added DIEA (87.6 mg, 678.1 mol, 118.1 μL, 3.0 equiv) and 2-chloro-N-[3-(3-chloro-4-cyano-phenoxy)-2,2,4,4-tetramethyl-cyclobutyl]pyrimidine-5-carboxamide (113.7 mg, 271.7 mol, 1.2 equiv). The mixture was stirred at 50° C. for 2 h. After filtration, the filtrate was purified by prep-HPLC (column: Phenomenex luna C18 150 mm*25 mm*10 μm; mobile phase: [water (FA)-ACN]; B %: 65%-95%, 10 min) to give a compound N-[3-(3-chloro-4-cyano-phenoxy)-2,2,4,4-tetramethyl-cyclobutyl]-2-[4-[2-[6-[4-(1-hydroxy-1-methyl-ethyl)-2-(6-methyl-7-oxo-1H-pyrrolo[2,3-c]pyridin-4-yl)phenoxy]-3-pyridyl]ethyl]-1-piperidyl]pyrimidine-5-carboxamide (60 mg, 29% yield, 95% purity) as a white solid. ¹H NMR (400 MHz, DMSO-d₆) δ 8.72 (s, 2H), 7.76-7.78 (m, 1H), 7.72 (d, J=8.68 Hz, 1H), 7.68 (d, J=2.32 Hz, 1H), 7.56 (dd, J=8.50, 2.38 Hz, 1H), 7.46 (dd, J=8.50, 2.38 Hz, 1H), 7.28 (d, J=2.81 Hz, 1H), 7.15-7.21 (m, 1H), 7.10-7.14 (m, 2H), 6.98 (dd, J=8.80, 2.45 Hz, 1H), 6.57 (d, J=8 0.44 Hz, 1H), 6.33 (d, J=2.81 Hz, 1H), 4.27 (s, 1H), 4.08-4.19 (m, 1H), 3.57 (s, 3H), 2.89-2.98 (m, 2H), 2.49-2.60 (m, 2H), 1.76-1.84 (m, 2H), 1.60 (s, 6H), 1.46-1.53 (m, 2H), 1.28 (s, 6H), 1.21 (s, 6H), 1.08-1.16 (m, 2H). LC-MS: MS (ES⁺): RT=2.73 min, m/z=869.4 [M+H⁺]; LCMS method: 25.

### Example 50—Synthesis of N-[3-(3-chloro-4-cyano-phenoxy)-2,2,4,4-tetramethyl-cyclobutyl]-2-[4-[1-[4-[4-(1-hydroxy-1-methyl-ethyl)-2-(6-methyl-7-oxo-1H-pyrrolo[2,3-c]pyridin-4-yl)phenoxy]cyclohexyl]azetidin-3-yl]oxy-1-piperidyl]pyrimidine-5-carboxamide (I-288)

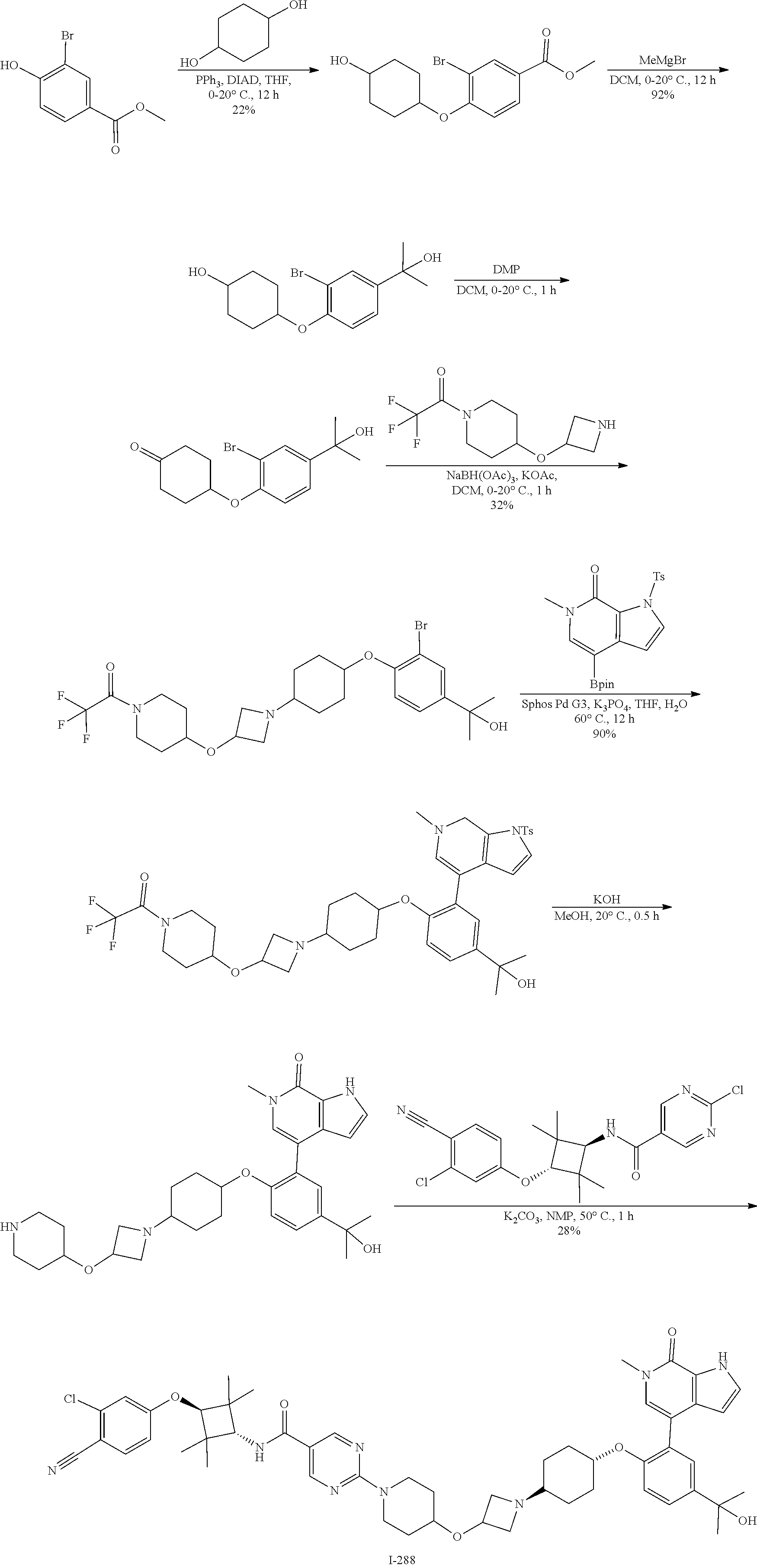

Step 1: Preparation of methyl 3-bromo-4-(4-hydroxycyclohexoxy)benzoate. To a solution of cyclohexane-1,4-diol (2 g, 17.22 mmol. 1.0 equiv), methyl 3-bromo-4-hydroxy-benzoate (3.98 g, 17.22 mmol, 1.0 equiv), PPh₃ (5.42 g, 20.66 mmol, 1.2 equiv) in THF (100 mL) was added DIAD (4.18 g, 20.66 mmol, 1.2 equiv) at 0° C. The mixture was stirred at 20° C. for 12 h. The residue was purified by prep-HPLC (column: Phenomenex luna C18 150 mm*40 mm*15 μm; mobile phase: [water(FA)-ACN]; B %: 37%-67%, 15 min) to give methyl 3-bromo-4-(4-hydroxycyclohexoxy)benzoate (1.25 g, 22% yield) as an off-white solid.

Step 2: Preparation of 4-[2-bromo-4-(1-hydroxy-1-methyl-ethyl)phenoxy]cyclohexanol. To a solution of methyl 3-bromo-4-(4-hydroxycyclohexoxy)benzoate (1.2 g, 3.65 mmol, 3.74 mL, 1.0 equiv) in DCM (20 mL) was added bromo(methyl)magnesium (3 M, 24.30 mL, 20.0 equiv) at 0° C. The mixture was stirred at 20° C. for 12 h. The reaction mixture was quenched with aq. NH₄Cl (30 mL). The mixture was extracted with DCM (50 mL*3), the combined organic was washed with brine (30 mL), dried over anhydrous Na₂SO₄, and the mixture was filtered and the filtrate was concentrated in vacuo. The residue was purified by silica gel chromatography (SiO₂, petroleum ether: ethyl acetate=10:1 to 0:1) to give 4-[2-bromo-4-(1-hydroxy-1-methyl-ethyl)phenoxy]cyclohexanol (1.1 g, 92% yield) as a yellow solid.

Step 3: Preparation of 4-[2-bromo-4-(1-hydroxy-1-methyl-ethyl)phenoxy]cyclohexanone. To a solution of 4-[2-bromo-4-(1-hydroxy-1-methyl-ethyl)phenoxy]cyclohexanol (500 mg, 1.52 mmol, 1.0 equiv) in DCM (50 mL) was added Dess-Martin Periodinane (1.29 g, 3.04 mmol, 2.0 equiv) at 0° C. The mixture was stirred at 20° C. for 1 h. The mixture was extracted with DCM (30 mL*3), the combined organic was washed with brine (80 mL), dried over anhydrous Na₂SO₄, and the mixture was filtered and the filtrate was concentrated in vacuo to give 4-[2-bromo-4-(1-hydroxy-1-methyl-ethyl)phenoxy]cyclohexanone (490 mg) as a yellow gum.

Step 4: Preparation of 1-[4-[1-[4-[2-bromo-4-(1-hydroxy-1-methyl-ethyl) phenoxy]cyclohexyl]azetidin-3-yl]oxy-1-piperidyl]-2,2,2-trifluoroethanone. To a solution of 1-[4-(azetidin-3-yloxy)-1-piperidyl]-2,2,2-trifluoro-ethanone (510 mg, 1.39 mmol, 1.0 equiv, TFA salt), 4-[2-bromo-4-(1-hydroxy-1-methyl-ethyl)phenoxy]cyclohexanone (460 mg, 1.41 mmol, 1.0 equiv) in DCM (30 mL) was added KOAc (344 mg, 3.51 mmol, 2.5 equiv) and NaBH(OAc)₃ (447 mg, 2.11 mmol, 1.5 equiv) at 0° C., and the mixture was stirred at 20° C. for 1 h. The reaction mixture was quenched with aq. NaHCO₃ (10 mL), the aqueous phase was extracted with DCM (50 mL*3). The combined organic phase was washed with brine (20 mL*2), dried with anhydrous Na₂SO₄, filtered and concentrated in vacuum. The residue was purified by prep-HPLC (column: Waters Xbridge 150 mm*25 mm*5 μm; mobile phase: [water(NH₄HCO₃)-ACN]; B %: 40%-70%, 10 mn) to give the desired product 1-[4-[1-[4-[2-bromo-4-(1-hydroxy-1-methyl-ethyl)phenoxy]cyclohexyl]azetidin-3-yl]oxy-1-piperidyl]-2,2,2-trifluoroethanone (250 mg, 32% yield) as a white solid.

Step 5: Preparation of 4-[5-(1-hydroxy-1-methyl-ethyl)-2-[4-[3-[[1-(2,2,2-trifluoroacetyl)-4-piperidyl]oxy]azetidin-1-yl]cyclohexoxy]phenyl]-6-methyl-1-(p-tolylsulfonyl)pyrrolo[2,3-c]pyridin-7-one. To a solution of 6-methyl-1-(p-tolylsulfonyl)-4-(4,4,5,5-tetramethyl-1,3,2-dioxaborolan-2-yl)pyrrolo[2,3-c]pyridin-7-one (182 mg, 425 μmol, 1.2 equiv) and 1-[4-[1-[4-[2-bromo-4-(1-hydroxy-1-methylethyl)phenoxy]cyclohexyl]azetidin-3-yl]oxy-1-piperidyl]-2,2,2-trifluoro-ethanone (200 mg, 354 μmol, 1.0 equiv) in THF (10 mL) was added H₂O(1 mL), SPhos Pd G3 (28 mg, 36 μmol, 0.1 equiv) and K₃PO₄ (150 mg, 709 μmol, 2.0 equiv). The mixture was stirred at 60° C. for 12 h. The residue was purified by prep-TLC (DCM:MeOH=10:1) to give 4-[5-(1-hydroxy-1-methyl-ethyl)-2-[4-[3-[[1-(2,2,2-trifluoroacetyl)-4-piperidyl]oxy]azetidin-1-yl]cyclohexoxy]phenyl]-6-methyl-1-(p-tolylsulfonyl)pyrrolo[2,3-c]pyridin-7-one (250 mg, 90% yield).

Step 6: Preparation of 4-[5-(1-hydroxy-1-methyl-ethyl)-2-[4-[3-(4-piperidyloxy)azetidin-1-yl]cyclohexoxy]phenyl]-6-methyl-1H pyrrolo[2,3-c]pyridin-7-one. To a solution of 4-[5-(1-hydroxy-1-methyl-ethyl)-2-[4-[3-[[1-(2,2,2-trifluoroacetyl)-4-piperidyl]oxy]azetidin-1-yl]cyclohexoxy]phenyl]-6-methyl-1-(p-tolylsulfonyl)pyrrolo[2,3-c]pyridin-7-one (250 mg, 318 μmol, 1.0 equiv) in MeOH (5 mL) was added KOH (357 mg, 6.37 mmol, 20.0 equiv). The mixture was stirred at 20° C. for 0.5 h. The mixture was extracted with DCM (30 mL*3), the combined organic was washed with brine (20 mL), dried over anhydrous Na₂SO₄, and the mixture was filtered and the filtrate was concentrated to give 4-[5-(1-hydroxy-1-methyl-ethyl)-2-[4-[3-(4-piperidyloxy)azetidin-1-yl]cyclohexoxy]phenyl]-6-methyl-1H pyrrolo[2,3-c]pyridin-7-one (170 mg) as a yellow gum.

Step 7: Preparation of N-[3-(3-chloro-4-cyano-phenoxy)-2,2,4,4-tetramethyl-cyclobutyl]-2-[4-[1-[4-[4-(1-hydroxy-1-methyl-ethyl)-2-(6-methyl-7-oxo-1H-pyrrolo[2,3-c]pyridin-4-yl)phenoxy]cyclohexyl]azetidin-3-yl]oxy-1-piperidyl]pyrimidine-5-carboxamide (I-288). To a solution of 4-[5-(1-hydroxy-1-methyl-ethyl)-2-[4-[3-(4-piperidyloxy)azetidin-1-yl]cyclohexoxy]phenyl]-6-methyl-1H-pyrrolo[2,3-c]pyridin-7-one (200 mg, 374 μmol, 1.0 equiv) in NMP (5 mL) was added K₂CO₃ (155 mg, 1.12 mmol, 3.0 equiv) and 2-chloro-N-[3-(3-chloro-4-cyano-phenoxy)-2,2,4,4-tetramethyl-cyclobutyl]pyrimidine-5-carboxamide (157 mg, 374 mol, 1.0 equiv). The mixture was stirred at 50° C. for 1 h. The aqueous phase was extracted with EA (20 mL*3). The combined organic phase was washed with brine (10 mL*2), dried with anhydrous Na₂SO₄, filtered and concentrated in vacuum. The residue was purified by prep-TLC (DCM:MeOH=10:1) to give the desired product N-[3-(3-chloro-4-cyano-phenoxy)-2,2,4,4-tetramethyl-cyclobutyl]-2-[4-[1-[4-[4-(1-hydroxy-1-methyl-ethyl)-2-(6-methyl-7-oxo-1H-pyrrolo[2,3-c]pyridin-4-yl)phenoxy]cyclohexyl]azetidin-3-yl]oxy-1-piperidyl]pyrimidine-5-carboxamide (55 mg, 15% yield) as an off-white solid. ¹H NMR (400 MHz, DMSO-d₆): δ 11.99-11.90 (m, 1H), 8.76 (s, 2H), 7.90 (d, J=8.8 Hz, 1H), 7.78-7.67 (m, 1H), 7.45 (d, J=2.5 Hz, 1H), 7.35 (dd, J=8.4, 2.4 Hz, 1H), 7.29-7.17 (m, 3H), 7.06-6.97 (m, 2H), 6.14 (d, J=2.0 Hz, 1H), 4.93 (s, 1H), 4.33-4.28 (m, 4H), 4.15-4.07 (m, 1H), 4.04 (d, J=9.4 Hz, 1H), 3.61-3.54 (m, 4H), 3.48 (d, J=5.2 Hz, 2H), 3.40-3.38 (m, 2H), 2.69 (d, J=5.6 Hz, 2H), 1.98-1.76 (m, 5H), 1.65-1.55 (m, 2H), 1.48-1.42 (m, 6H), 1.39-1.32 (m, 2H), 1.26-1.20 (m, 8H), 1.11 (s, 6H), 1.07-0.97 (m, 2H) LC-MS: MS (ES⁺): RT=1.997 min, m/z=917.4 [M+H⁺]; LCMS method: 25.

### Example 51—Synthesis of N-[3-(3-chloro-4-cyano-phenoxy)-2,2,4,4-tetramethyl-cyclobutyl]-2-[4-[2-[4-[5-ethylsulfonyl-2-(6-methyl-7-oxo-1H-pyrrolo[2,3-c]pyridin-4-yl)phenyl]-5-oxo-pyrazin-2-yl]ethyl]-1-piperidyl]pyrimidine-5-carboxamide (I-292)

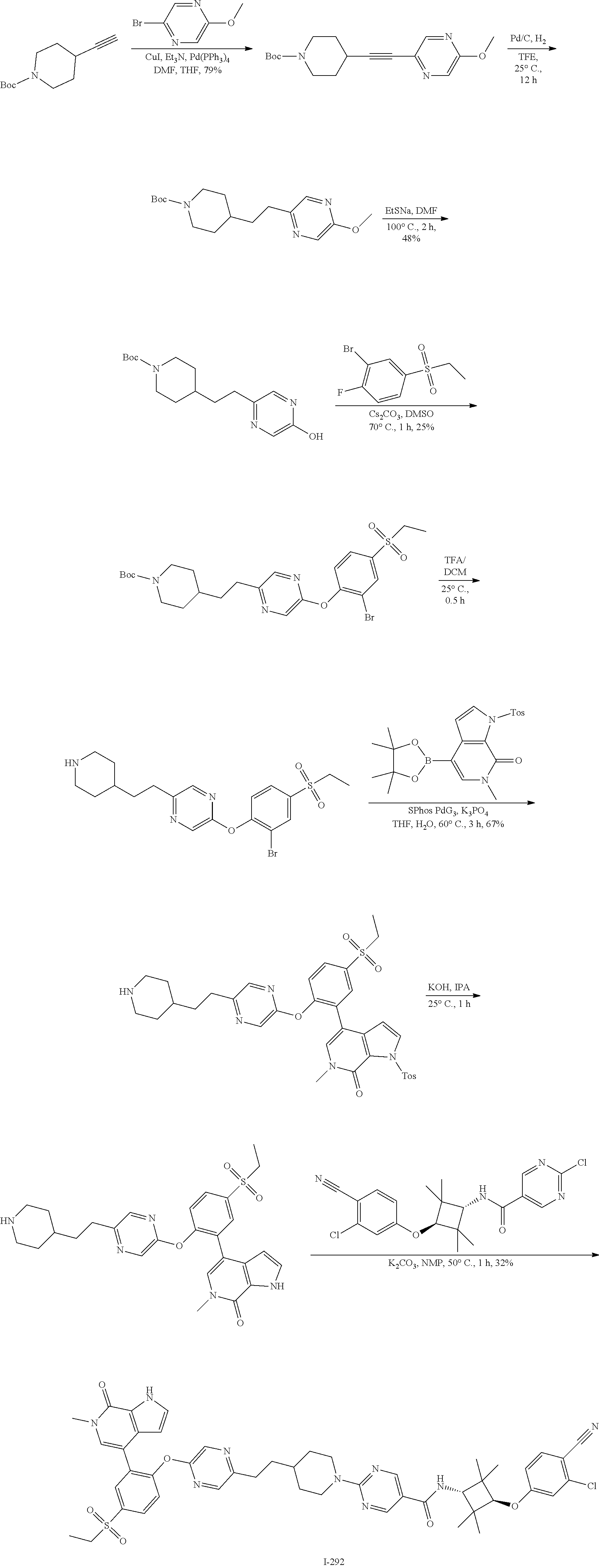

Step 1: Preparation of tert-butyl 4-[2-(5-methoxypyrazin-2-yl)ethynyl]piperidine-1-carboxylate. To a solution of 2-bromo-5-methoxy-pyrazine (3 g, 15.8 mmol, 1.0 equiv), tert-butyl 4-ethynylpiperidine-1-carboxylate (3.6 g, 17.4 mmol, 1.1 equiv) in DMF (20 mL) and THF (20 mL) was added CuI (302.9 mg, 1.9 mmol, 0.1 equiv) and Et₃N (12.1 g, 119.7 mmol, 16.6 mL, 7.5 equiv), Pd(PPh₃)₄ (1.8 g, 1.5 mmol, 0.1 equiv). The mixture was stirred at 80° C. for 2 h. The residue was purified by column chromatography (SiO₂, petroleum ether: ethyl acetate=20:1 to 6:1) to give the compound tert-butyl 4-[2-(5-methoxypyrazin-2-yl)ethynyl]piperidine-1-carboxylate (4 g, 79% yield) as a colorless oil.

Step 2: Preparation of tert-butyl 4-[2-(5-methoxypyrazin-2-yl) ethyl]piperidine-1-carboxylate. To a solution of tert-butyl 4-[2-(5-methoxypyrazin-2-yl) ethynyl]piperidine-1-carboxylate (4 g, 12.6 mmol, 1 equiv) in CF₃CH₂OH (40 mL) was added Pd/C (2.0 g, 10% purity). The mixture was stirred at 25° C. for 12 h under H₂ (15 psi). The reaction mixture filtered and concentrated under reduced pressure to give the compound tert-butyl 4-[2-(5-methoxypyrazin-2-yl) ethyl]piperidine-1-carboxylate (3.8 g, 94% yield) as a colorless oil.

Step 3: Preparation of tert-butyl 4-[2-(5-hydroxypyrazin-2-yl) ethyl]piperidine-1-carboxylate. To a solution of tert-butyl 4-[2-(5-methoxypyrazin-2-yl) ethyl]piperidine-1-carboxylate (2 g, 6.2 mmol, 1.0 equiv) in DMF (20 mL) was added ethylsulfanylsodium (2.9 g, 24.9 mmol, 4.0 equiv). The mixture was stirred at 100° C. for 2 h. The mixture was adjusted to pH=4˜5 with AcOH (5 mL). The mixture was added to H₂O (50 mL) and extracted with EA (50 mL×3). The combined organic layers were dried over anhydrous Na₂SO₄, filtered and concentrated under reduced pressure to give a residue. The residue was purified by prep-HPLC (column: Waters Xbridge C18 150 mm*50 mm*m; mobile phase: [water (NH₄HCO₃)-ACN]; B %: 24%-54%, 10 min) to give a compound tert-butyl 4-[2-(5-hydroxypyrazin-2-yl) ethyl]piperidine-1-carboxylate (0.8 g, 42 % yield) as a white solid.

Step 4: Preparation of tert-butyl 4-[2-[5-(2-bromo-4-ethylsulfonyl-phenoxy) pyrazin-2-yl]ethyl]piperidine-1-carboxylate. To a solution of tert-butyl 4-[2-(5-hydroxypyrazin-2-yl) ethyl]piperidine-1-carboxylate (0.77 g, 2.50 mmol, 1 equiv), 2-bromo-4-ethylsulfonyl-1-fluoro-benzene (736.3 mg, 2.7 mmol, 1.1 equiv) in DMSO (3 mL) was added Cs₂CO₃ (2.5 g, 7.5 mmol, 3.0 equiv). The mixture was stirred at 70° C. for 1 h. After filtration, the filtrate was purified by prep-HPLC (column: Waters Xbridge C18 150 mm*50 mm*10 μm; mobile phase: [water (NH₄HCO₃)-ACN]; B %: 44%-74%, 10 min) to give a compound tert-butyl 4-[2-[5-(2-bromo-4-ethylsulfonyl-phenoxy) pyrazin-2-yl]ethyl]piperidine-1-carboxylate (0.35 g, 25% yield) as a white solid.

Step 5: Preparation of 2-(2-bromo-4-ethylsulfonyl-phenoxy)-5-[2-(4-piperidyl) ethyl]pyrazine. To a solution of tert-butyl 4-[2-[5-(2-bromo-4-ethylsulfonyl-phenoxy) pyrazin-2-yl]ethyl]piperidine-1-carboxylate (0.25 g, 450.8 mol, 1.0 equiv) in DCM (1 mL) was added TFA (0.5 mL). The mixture was stirred at 25° C. for 0.5 h. The reaction mixture was concentrated under reduced pressure to remove solvent to give compound 2-(2-bromo-4-ethylsulfonyl-phenoxy)-5-[2-(4-piperidyl) ethyl]pyrazine (256 mg, 99% yield, TFA salt) as a colorless oil.

Step 6: Preparation of compound 4-[5-ethylsulfonyl-2-[5-[2-(4-piperidyl)ethyl]pyrazin-2-yl]oxy-phenyl]-6-methyl-1-(p-tolylsulfonyl)pyrrolo[2,3-c]pyridin-7-one. A mixture of 2-(2-bromo-4-ethylsulfonyl-phenoxy)-5-[2-(4-piperidyl)ethyl]pyrazine (0.25 g, 439.8 μmol, 1.0 equiv, TFA salt), 6-methyl-1-(p-tolylsulfonyl)-4-(4,4,5,5-tetramethyl-1,3,2-dioxaborolan-2-yl)pyrrolo[2,3-c]pyridin-7-one (244.9 mg, 571.8 mol, 1.3 equiv), K₃PO₄ (280.1 mg, 1.3 mmol, 3.0 equiv) and SPhos PdG₃ (34.3 mg, 43.9 mol, 0.1 equiv) in THF (2.5 mL) and H₂O (0.5 mL) was degassed and purged with N₂ three times, and then the mixture was stirred at 60° C. for 3 h under an N₂ atmosphere. After concentration, the residue was purified by prep-HPLC (column: Phenomenex C18 75 mm*30 mm*3 am; mobile phase: [water (FA)-ACN]; B %: 15%-45%, 7 min) to give a compound 4-[5-ethylsulfonyl-2-[5-[2-(4-piperidyl)ethyl]pyrazin-2-yl]oxy-phenyl]-6-methyl-1-(p-tolylsulfonyl)pyrrolo[2,3-c]pyridin-7-one (0.2 g, 67% yield) as a white solid.

Step 7: Preparation of 4-[5-ethylsulfonyl-2-[5-[2-(4-piperidyl) ethyl]pyrazin-2-yl]oxy-phenyl]-6-methyl-11H-pyrrolo [2, 3-c]pyridin-7-one. To a solution of 4-[5-ethylsulfonyl-2-[5-[2-(4-piperidyl) ethyl]pyrazin-2-yl]oxy-phenyl]-6-methyl-1-(p-tolylsulfonyl) pyrrolo [2, 3-c]pyridin-7-one (0.2 g, 295.9 mol, 1.0 equiv) in IPA (2 mL) was added KOH (1 M, 7.99 mL, 15.0 equiv). The mixture was stirred at 25° C. for 12 h. The reaction mixture was adjusted by AcOH to pH=5-6 and freeze-dried to give a compound 4-[5-ethylsulfonyl-2-[5-[2-(4-piperidyl) ethyl]pyrazin-2-yl]oxy-phenyl]-6-methyl-1H-pyrrolo [2, 3-c]pyridin-7-one (152 mg, 98% yield) as a colorless oil.

Step 8: Preparation of N-[3-(3-chloro-4-cyano-phenoxy)-2,2,4,4-tetramethyl-cyclobutyl]-2-[4-[2-[4-[5-ethylsulfonyl-2-(6-methyl-7-oxo-1H-pyrrolo[2,3-c]pyridin-4-yl)phenyl]-5-oxo-pyrazin-2-yl]ethyl]-1-piperidyl]pyrimidine-5-carboxamide (I-292). To a solution of 4-[4-ethylsulfonyl-2-[2-oxo-5-[2-(4-piperidyl)ethyl]pyrazin-1-yl]phenyl]-6-methyl-1H-pyrrolo[2,3-c]pyridin-7-one (0.1 g, 157.2 μmol, 1.0 equiv, TFA salt), 2-chloro-N-[3-(3-chloro-4-cyano-phenoxy)-2,2,4,4-tetramethyl-cyclobutyl]pyrimidine-5-carboxamide (72.5 mg, 173.0 μmol, 1.1 equiv) in NMP (3 mL) was added K₂CO₃ (65.2 mg, 471.6 μmol, 3.0 equiv). The mixture was stirred at 50° C. for 1 h. After filtration, the filtrate was purified by prep-HPLC (column: Phenomenex Luna C18 150 mm*25 mm*10 μm; mobile phase: [water (FA)-ACN]; B %: 57%-87%, 10 min) to give a compound N-[3-(3-chloro-4-cyano-phenoxy)-2,2,4,4-tetramethyl-cyclobutyl]-2-[4-[2-[4-[5-ethylsulfonyl-2-(6-methyl-7-oxo-1H-pyrrolo[2,3-c]pyridin-4-yl)phenyl]-5-oxo-pyrazin-2-yl]ethyl]-1-piperidyl]pyrimidine-5-carboxamide (47.1 mg, 32% yield) as a white solid. ¹H NMR (400 MHz, DMSO-d₆) δ 12.06 (s, 1H), 8.74 (s, 2H), 8.24 (s, 1H), 8.06 (s, 1H), 7.94-8.01 (m, 1H), 7.85-7.94 (m, 2H), 7.70 (d, J=9.17 Hz, 1H), 7.53 (d, J=8.56 Hz, 1H), 7.37 (d, J=2.57 Hz, 1H), 7.26 (s, 1H), 7.21 (d, J=2.32 Hz, 1H), 7.00 (dd, J=8.68, 2.32 Hz, 1H), 5.98-6.20 (m, 1H), 4.73 (d, J=12.84 Hz, 2H), 4.29 (s, 1H), 4.03 (d, J=9.17 Hz, 1H), 3.51 (s, 3H), 3.39-3.42 (m, 1H), 2.90-2.95 (m, 2H), 2.70-2.74 (m, 2H), 1.73-1.83 (m, 2H), 1.52-1.61 (m, 2H), 0.96-1.27 (m, 19H). LC-MS: MS (ES⁺): RT=2.65 min, m/z=904.4 [M+H]; LCMS method: 25.

### Example 52—Synthesis of N-[3-(3-chloro-4-cyano-phenoxy)-2,2,4,4-tetramethyl-cyclobutyl]-2-[2-[3-[4-ethylsulfonyl-2-(6-methyl-7-oxo-1Hpyrrolo[2,3-c]pyridin-4-yl)phenoxy]cyclobutoxy]-7-azaspiro[3.5]nonan-7-yl]pyrimidine-5-carboxamide (I-295)

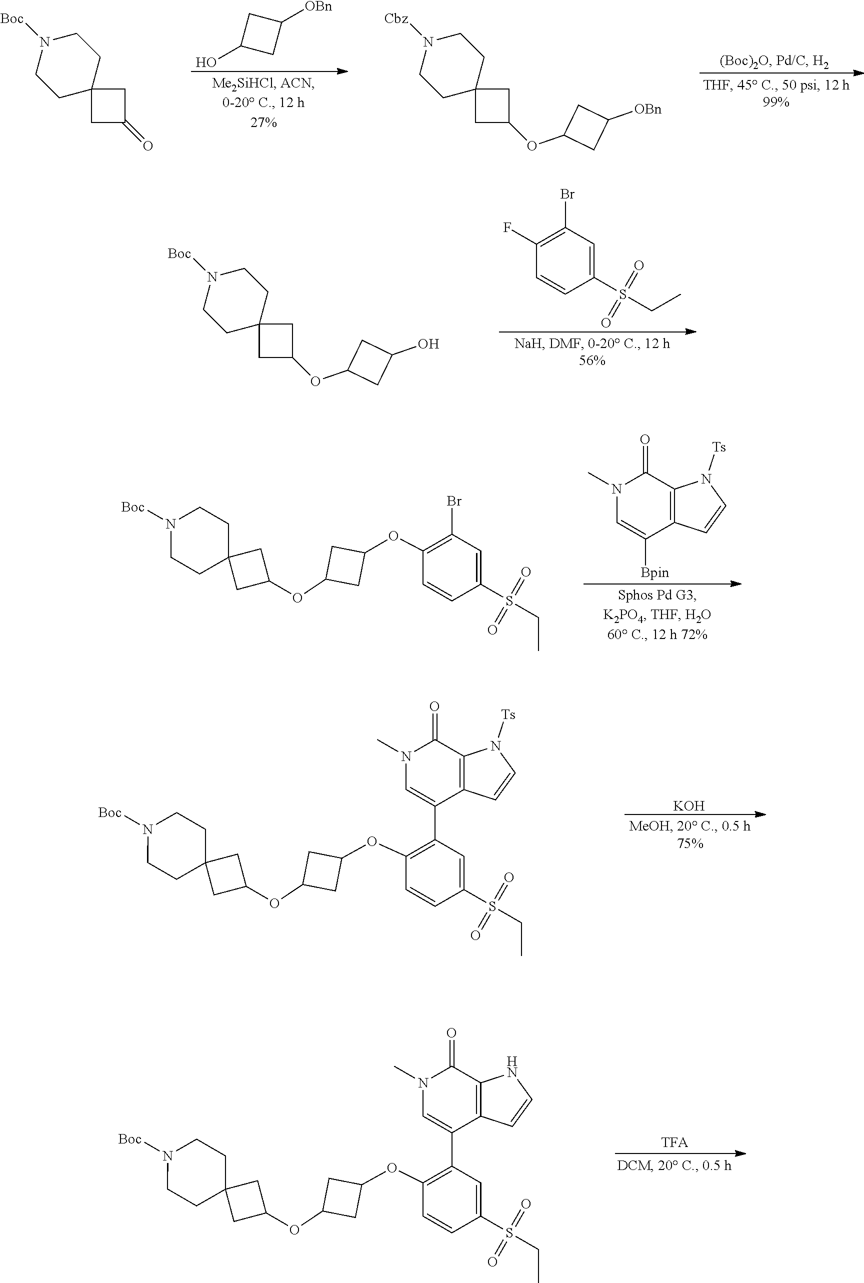

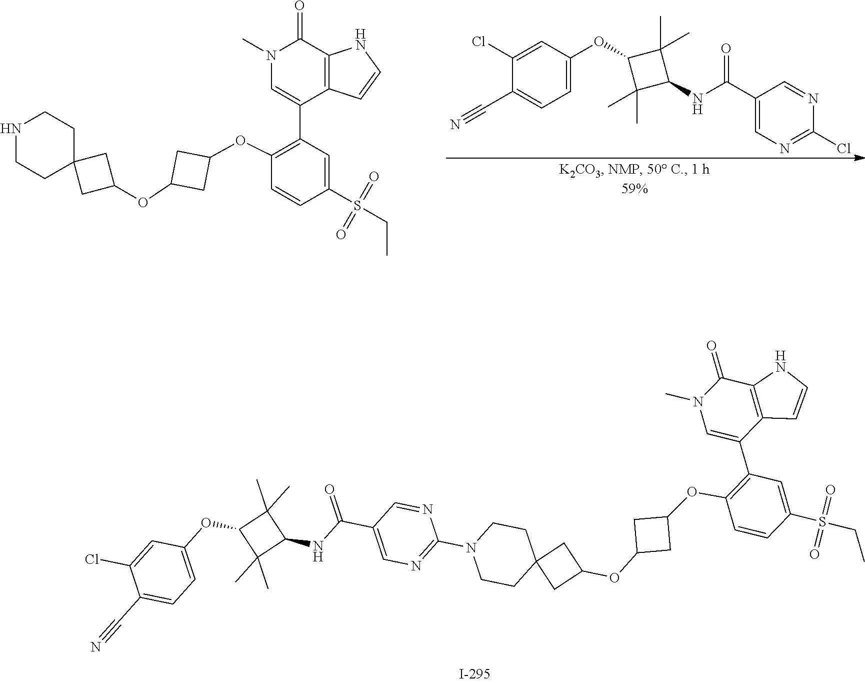 [SMILES: CCS(=O)(=O)c1ccc(OC2CC(OC3CC4(CCNCC4)C3)C2)c(-c2cn(C)c(=O)c3[nH]ccc23)c1 ; CC1(C)[C@H](NC(=O)c2cnc(Cl)nc2)C(C)(C)[C@H]1Oc1ccc(C#N)c(Cl)c1 ; CCS(=O)(=O)c1ccc(OC2CC(OC3CC4(CCN(c5ncc(C(=O)N[C@H]6C(C)(C)[C@H](Oc7ccc(C#N)c(Cl)c7)C6(C)C)cn5)CC4)C3)C2)c(-c2cn(C)c(=O)c3[nH]ccc23)c1]

Step 1: Preparation of benzyl 2-(3-benzyloxycyclobutoxy)-7-azaspiro[3.5]nonane-7-carboxylate. To a solution of 3-benzyloxycyclobutanol (1.43 g, 8.05 mmol, 1.1 equiv) and benzyl 2-oxo-7-azaspiro[3.5]nonane-7-carboxylate (2 g, 7.32 mmol, 1.0 equiv) in ACN (20 mL) was added chloro(dimethyl)silane (1.38 g, 14.63 mmol, 2.0 equiv) at 0° C. The mixture was stirred at 20° C. for 12 h. The residue was purified by prep-HPLC (column: Phenomenex luna C18 150 mm*40 mm*15 μm; mobile phase: [water(FA)-ACN]; B %: 60%-90%, 10 min) to give benzyl 2-(3-benzyloxycyclobutoxy)-7-azaspiro[3.5]nonane-7-carboxylate (850 mg, 27% yield) as a yellow gum.

Step 2: Preparation of tert-butyl 2-(3-hydroxycyclobutoxy)-7-azaspiro[3.5]nonane-7-carboxylate. To a solution of benzyl 2-(3-benzyloxycyclobutoxy)-7-azaspiro[3.5]nonane-7-carboxylate (650 mg, 1.49 mmol, 1.0 equiv) and (Boc)₂₀ (358 mg, 1.64 mmol, 1.1 equiv) in THF (50 mL) was added Pd/C (100 mg, 10% purity) under an N₂ atmosphere. The suspension was degassed and purged with H₂ three times. The mixture was stirred under H₂ (50 psi) at 45° C. for 12 h. The residue was purified by column chromatography (SiO₂, Petroleum ether: ethyl acetate=5:1 to 0:1) to give tert-butyl 2-(3-hydroxycyclobutoxy)-7-azaspiro[3.5]nonane-7-carboxylate (460 mg, 99% yield) as a yellow gum.

Step 3: Preparation of tert-butyl 2-[3-(2-bromo-4-ethylsulfonyl-phenoxy) cyclobutoxy]-7-azaspiro[3.5]nonane-7-carboxylate. To a solution of tert-butyl 2-(3-hydroxycyclobutoxy)-7-azaspiro[3.5]nonane-7-carboxylate (50 mg, 160 μmol, 1.0 equiv) in THF (3 mL) was added NaH (8 mg, 193 mol, 60% purity, 1.2 equiv) at 0° C., the mixture was then stirred at 20° C. for 0.5 h, and then 2-bromo-4-ethylsulfonyl-1-fluoro-benzene (43 mg, 160 μmol, 1.0 equiv) was added. The mixture was stirred at 20° C. for 11.5 h. The residue was purified by prep-TLC (SiO₂, Petroleum ether: ethyl acetate=1:1) to give tert-butyl 2-[3-(2-bromo-4-ethylsulfonyl-phenoxy)cyclobutoxy]-7-azaspiro[3.5]nonane-7-carboxylate (50 mg, 56% yield) as a yellow gum.

Step 4: Preparation of tert-butyl 2-[3-[4-ethylsulfonyl-2-[6-methyl-7-oxo-1-(p-tolylsulfonyl)pyrrolo[2,3-c]pyridin-4-yl]phenoxy]cyclobutoxy]-7-azaspiro[3.5]nonane-7-carboxylate. To a solution of 6-methyl-1-(p-tolylsulfonyl)-4-(4,4,5,5-tetramethyl-1,3,2-dioxaborolan-2-yl)pyrrolo[2,3-c]pyridin-7-one (46 mg, 107 μmol, 1.2 equiv) and tert-butyl 2-[3-(2-bromo-4-ethylsulfonyl-phenoxy)cyclobutoxy]-7-azaspiro[3.5]nonane-7-carboxylate (50 mg, 89 μmol, 1.0 equiv) in THF (5 mL) was added H₂O (1 mL), SPhos Pd G3 (7 mg, 9 μmol, 0.1 equiv) and K₃PO₄ (38 mg, 179 mol, 2.0 equiv). The mixture was stirred at 60° C. for 12 h. The residue was purified by prep-HPLC (SiO₂, DCM:MeOH=20:1) to give tert-butyl 2-[3-[4-ethylsulfonyl-2-[6-methyl-7-oxo-1-(p-tolylsulfonyl)pyrrolo[2,3-c]pyridin-4-yl]phenoxy]cyclobutoxy]-7-azaspiro[3.5]nonane-7-carboxylate (50 mg, 72% yield) as a yellow gum.

Step 5: Preparation of tert-butyl 2-[3-[4-ethylsulfonyl-2-(6-methyl-7-oxo-1H-pyrrolo[2,3-c]pyridin-4-yl)phenoxy]cyclobutoxy]-7-azaspiro[3.5]nonane-7-carboxylate. To a solution of tert-butyl 2-[3-[4-ethylsulfonyl-2-[6-methyl-7-oxo-1-(p-tolylsulfonyl) pyrrolo[2,3-c]pyridin-4-yl]phenoxy]cyclobutoxy]-7-azaspiro[3.5]nonane-7-carboxylate (50 mg, 64 mol, 1.0 equiv) in MeOH (5 mL) was added KOH (72 mg, 1.28 mmol, 20.0 equiv). The mixture was stirred at 20° C. for 0.5 h. The mixture was extracted with DCM (30 mL*3), the combined organic was washed with brine (30 mL), dried over anhydrous Na₂SO₄, the mixture was filtered and the filtrate was concentrated in vacuo to give tert-butyl 2-[3-[4-ethylsulfonyl-2-(6-methyl-7-oxo-1H-pyrrolo[2,3-c]pyridin-4-yl)phenoxy]cyclobutoxy]-7-azaspiro[3.5]nonane-7-carboxylate (30 mg, 75% yield) as a yellow gum.

Step 6: Preparation of 4-[2-[3-(7-azaspiro[3.5]nonan-2-yloxy)cyclobutoxy]-5-ethylsulfonyl-phenyl]-6-methyl-1H-pyrrolo[2,3-c]pyridin-7-one. To a solution of tert-butyl 2-[3-[4-ethylsulfonyl-2-(6-methyl-7-oxo-1H-pyrrolo[2,3-c]pyridin-4-yl)phenoxy]cyclobutoxy]-7-azaspiro[3.5]nonane-7-carboxylate (30 mg, 48 mol, 1.0 equiv) in DCM (1 mL) was added TFA (0.3 mL). The mixture was stirred at 20° C. for 0.5 h. The mixture was concentrated in vacuo to give the desired product 4-[2-[3-(7-azaspiro[3.5]nonan-2-yloxy)cyclobutoxy]-5-ethylsulfonyl-phenyl]-6-methyl-1H-pyrrolo[2,3-c]pyridin-7-one (30 mg, TFA salt) as a yellow gum.

Step 7: Preparation of N-[3-(3-chloro-4-cyano-phenoxy)-2,2,4,4-tetramethyl-cyclobutyl]-2-[2-[3-[4-ethylsulfonyl-2-(6-methyl-7-oxo-1Hpyrrolo[2,3-c]pyridin-4-yl)phenoxy]cyclobutoxyl-7-azaspiro[3.5]nonan-7-yl]pyrimidine-5-carboxamide (I-295). To a solution of 4-[2-[3-(7-azaspiro[3.5]nonan-2-yloxy)cyclobutoxy]-5-ethylsulfonyl-phenyl]-6-methyl-1Hpyrrolo[2,3-c]pyridin-7-one (80 mg, 125 μmol, 1.0 equiv, TFA salt) in NMP (5 mL) was added K₂CO₃ (52 mg, 375 μmol, 3.0 equiv) and 2-chloro-N-[3-(3-chloro-4-cyano-phenoxy)-2,2,4,4-tetramethyl-cyclobutyl]pyrimidine-5-carboxamide (52 mg, 125 mol, 1.0 equiv). The mixture was stirred at 50° C. for 1 h. The residue was purified by prep-HPLC (column: Phenomenex luna C18 150 mm*25 mm*10 μm;mobile phase:[water(FA)-ACN]; B %: 71%-100%, 10 min) to give N-[3-(3-chloro-4-cyano-phenoxy)-2,2,4,4-tetramethyl-cyclobutyl]-2-[2-[3-[4-ethylsulfonyl-2-(6-methyl-7-oxo-1Hpyrrolo[2,3-c]pyridin-4-yl)phenoxy]cyclobutoxy]-7-azaspiro[3.5]nonan-7-yl]pyrimidine-5-carboxamide (70 mg. 59% yield). ¹H NMR (400 MHz, DMSO-d₆): δ 12.10-12.01 (m, 1H), 8.78-8.68 (m, 2H), 7.93-7.87 (m, 1H), 7.85-7.76 (m, 2H), 7.73-7.67 (m, 1H), 7.37-7.27 (m, 2H), 7.23-7.14 (m, 2H), 7.05-6.97 (m, 1H), 6.16-6.04 (m, 1H), 4.56-4.43 (m, 1H), 4.32-4.24 (m, 1H), 4.06-3.93 (m, 2H), 3.82-3.65 (m, 5H), 3.58 (s, 3H), 3.30-3.25 (m, 2H), 2.91-2.76 (m, 2H), 2.21-2.12 (m, 2H), 1.93-1.81 (m, 2H), 1.71-1.62 (m, 2H), 1.56-1.45 (m, 4H), 1.21 (s, 6H), 1.16-1.09 (m, 9H). LC-MS: MS (ES⁺): RT=2.835 min, m/z=908.4 [M+H⁺]; LCMS method: 25.

### Example 53—Synthesis of N-[3-(3-chloro-4-cyano-phenoxy)-2,2,4,4-tetramethyl-cyclobutyl]-2-[4-[[5-[4-(1-hydroxy-1-methyl-ethyl)-2-(6-methyl-7-oxo-1H-pyrrolo[2,3-c]pyridin-4-yl)phenoxy]-2-pyridyl]oxy]-1-piperidyl]pyrimidine-5-carboxamide (I-297)

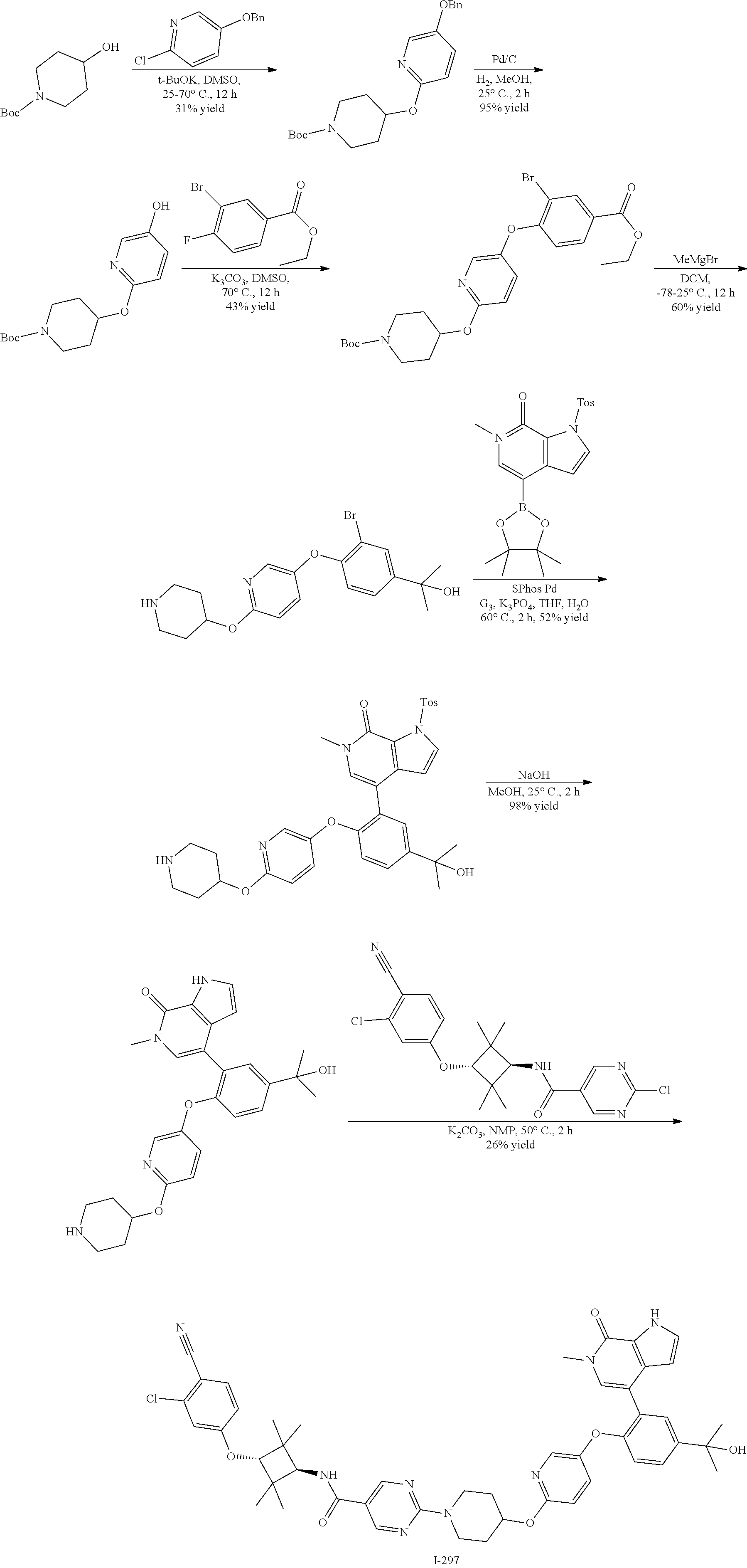

Step 1: Preparation of tert-butyl 4-[(5-benzyloxy-2-pyridyl) oxy]piperidine-1-carboxylate. To a solution of t-BuOK (1 M, 99 mL, 2.0 equiv) in DMSO (50 mL) was added tert-butyl 4-hydroxypiperidine-1-carboxylate (10.0 g, 49.7 mmol, 1.0 equiv) and the reaction mixture was stirred at 25° C. for 1 h. 5-benzyloxy-2-chloro-pyridine (16.4 g, 74.5 mmol, 5 mL, 1.5 equiv) was added and the mixture was stirred at 70° C. for 12 h. The reaction mixture was quenched with saturated aqueous NH₄Cl (100 mL) solution at 0° C. dropwise. And the resulting mixture was extracted with ethyl acetate (30 mL×3). The combined organic layers were dried over anhydrous Na₂SO₄, filtered and concentrated in vacuum. The residue was purified by column chromatography (SiO₂, Petroleum ether: ethyl acetate=10:1 to 3:1). The compound tert-butyl 4-[(5-benzyloxy-2-pyridyl) oxy]piperidine-1-carboxylate (6.0 g, 15.6 mmol, 310% yield) was obtained as a white solid.

Step 2: Preparation of tert-butyl 4-[(5-hydroxy-2-pyridyl) oxy]piperidine-1-carboxylate. To a solution of tert-butyl 4-[(5-benzyloxy-2-pyridyl)oxy]piperidine-1-carboxylate (3.00 g, 7.80 mmol, 1.0 equiv) in MeOH (60 mL) was added Pd/C (1.50 g, 1.40 mmol, 10% purity, 1.8e-1 equiv) under N₂ atmosphere. The suspension was degassed and purged with H₂ three times. The mixture was stirred under H₂ (16.0 mg, 7.80 mmol, 1.0 equiv) at 25° C. for 2 h. The reaction mixture was filtered and concentrated under reduced pressure to give a residue. The compound tert-butyl 4-[(5-hydroxy-2-pyridyl) oxy]piperidine-1-carboxylate (2.20 g, 7.50 mmol, 95% yield) was obtained as a white oil.

Step 3: Preparation of tert-butyl 4-[[5-(2-bromo-4-ethoxycarbonyl-phenoxy)-2-pyridyl]oxy]piperidine-1-carboxylate. K₂CO₃ (610 mg, 4.40 nmol, 1.0 equiv), tert-butyl 4-[(5-hydroxy-2-pyridyl) oxy]piperidine-1-carboxylate (1.30 g, 4.40 mmol, 1.0 equiv), and ethyl 3-bromo-4-fluoro-benzoate (1.60 g, 6.60 mmol, 1.5 equiv) were added into a DMSO (12 mL). The mixture was stirred at 70° C. for 12 h. The reaction mixture was quenched by addition H₂O (100 mL), and then diluted with brine (10 mL) and extracted with EA (30 mL×3). The combined organic layers were washed with brine (30 mL), dried over Na₂SO₄, filtered and concentrated under reduced pressure to give a residue. The residue was purified by column chromatography (SiO₂, Petroleum ether:ethyl acetate=10:1 to 3:1). The compound tert-butyl 4-[[5-(2-bromo-4-ethoxycarbonyl-phenoxy)-2-pyridyl]oxy]piperidine-1-carboxylate (1.00 g, 1.90 mmol, 43% yield) was obtained as a white solid.

Step 4: Preparation of 2-[3-bromo-4-[[6-(4-piperidyloxy)-3-pyridyl]oxy]phenyl]propan-2-ol. To a solution of tert-butyl 4-[[5-(2-bromo-4-ethoxycarbonyl-phenoxy)-2-pyridyl]oxy]piperidine-1-carboxylate (1.00 g, 1.90 mmol, 1.0 equiv) in DCM (10 mL) was added MeMgBr (3 M, 10 mL, 15.0 equiv) at −78° C. The mixture was stirred at 25° C. for 12 h. The reaction mixture was quenched by addition NH₄Cl (10 mL) at −78° C., and then diluted with brine (10 mL) and extracted with EA (10 mL×3). The combined organic layers were washed with brine (10 mL), dried over Na₂SO₄, filtered and concentrated under reduced pressure to give a residue. The residue was purified by prep-HPLC (column: Phenomenex luna C18 150 mm×40 mm×15 um; mobile phase: [water (FA)-ACN]; B %: 10%-40%, 15 min). 2-[3-bromo-4-[[6-(4-piperidyloxy)-3-pyridyl]oxy]phenyl]propan-2-ol (475 mg, 1.20 mmol, 60% yield) was obtained as a white solid.

Step 5: Preparation of 4-[5-(1-hydroxy-1-methyl-ethyl)-2-[[6-(4-piperidyloxy)-3-pyridyl]oxy]phenyl]-6-methyl-1-(p-tolylsulfonyl) pyrrolo [2, 3-c]pyridin-7-one. A mixture of 6-methyl-1-(p-tolylsulfonyl)-4-(4,4,5,5-tetramethyl-1,3,2-dioxaborolan-2-yl)pyrrolo[2,3-c]pyridin-7-one (250 mg, 584 mol, 1.3 equiv), 2-[3-bromo-4-[[6-(4-piperidyloxy)-3-pyridyl]oxy]phenyl]propan-2-ol (182 mg, 449 μmol, 1.0 equiv), SPhos Pd G3 (61.0 mg, 78.6 mol, 1.8e-1 equiv) and added K₃PO₄ (288 mg, 1.40 mmol, 3.0 equiv) in THF (7 mL) and water (1.4 mL) was degassed and purged with N₂ three times, and then the mixture was stirred at 60° C. for 2 h under N₂ atmosphere. The reaction mixture was filtered and concentrated under reduced pressure to give a residue. The residue was purified by prep-HPLC (column: Phenomenex Luna C18 150 mm×25 mm×10 um; mobile phase: [water (FA)-ACN]; B %: 17%-47%, 10 min). 4-[5-(1-hydroxy-1-methyl-ethyl)-2-[[6-(4-piperidyloxy)-3-pyridyl]oxy]phenyl]-6-methyl-1-(p-tolylsulfonyl) pyrrolo [2, 3-c]pyridin-7-one (148 mg, 236 umol, 52% yield) was obtained as a white solid.

Step 6: Preparation of 4-[5-(1-hydroxy-1-methyl-ethyl)-2-[[6-(4-piperidyloxy)-3-pyridyl]oxy]phenyl]-6-methyl-1H-pyrrolo [2, 3-c]pyridin-7-one. To a solution of 4-[5-(1-hydroxy-1-methyl-ethyl)-2-[[6-(4-piperidyloxy)-3-pyridyl]oxy]phenyl]-6-methyl-1-(p-tolylsulfonyl)pyrrolo[2,3-c]pyridin-7-one (148 mg, 235 mol, 1.0 equiv) in MeOH (2 mL) was added NaOH (1 M, 3.5 mL, 15.0 equiv). The mixture was stirred at 25° C. for 2 h. The reaction mixture was filtered and concentrated under reduced pressure to give a residue. 4-[5-(1-hydroxy-1-methyl-ethyl)-2-[[6-(4-piperidyloxy)-3-pyridyl]oxy]phenyl]-6-methyl-1H-pyrrolo [2, 3-c]pyridin-7-one (110 mg, 232 μmol, 98% yield) was obtained as a white solid.

Step 7: Preparation of N-[3-(3-chloro-4-cyano-phenoxy)-2,2,4,4-tetramethyl-cyclobutyl]-2-[4-[[5-[4-(1-hydroxy-1-methyl-ethyl)-2-(6-methyl-7-oxo-1H-pyrrolo[2,3-c]pyridin-4-yl)phenoxy]-2-pyridyl]oxy]-1-piperidyl]pyrimidine-5-carboxamide (I-297). A mixture of 4-[5-(1-hydroxy-1-methyl-ethyl)-2-[[6-(4-piperidyloxy)-3-pyridyl]oxy]phenyl]-6-methyl-1H-pyrrolo[2,3-c]pyridin-7-one (55.00 mg, 115.9 μmol, 1.0 equiv), 2-chloro-N-[3-(3-chloro-4-cyano-phenoxy)-2,2,4,4-tetramethyl-cyclobutyl]pyrimidine-5-carboxamide (48.00 mg, 115.9 μmol, 1.0 equiv), K₂CO₃ (32.00 mg, 231.8 mol, 2.0 equiv) in NMP (2 mL) was degassed and purged with N₂ three times, and then the mixture was stirred at 50° C. for 2 h under an N₂ atmosphere. The reaction mixture was filtered and concentrated under reduced pressure to give a residue. The residue was purified by prep-HPLC (column: Phenomenex luna C18 150 mm×25 mm×10 um; mobile phase: [water (FA)-ACN]; B %: 70%-100%, 10 min). N-[3-(3-chloro-4-cyano-phenoxy)-2,2,4,4-tetramethyl-cyclobutyl]-2-[4-[[5-[4-(1-hydroxy-1-methyl-ethyl)-2-(6-methyl-7-oxo-1H-pyrrolo[2,3-c]pyridin-4-yl)phenoxy]-2-pyridyl]oxy]-1-piperidyl]pyrimidine-5-carboxamide (26.74 mg, 31.2 μmol, 26% yield) was obtained as a white solid. ¹H NMR: (400 MHz, DMSO-d₆) δ=12.02 (m, 1H), 8.78 (s, 2H), 7.91 (d, J=8.8 Hz, 1H), 7.84-7.72 (m, 2H), 7.57 (d, J=2.4 Hz, 1H), 7.47-7.33 (m, 2H), 7.30-7.24 (m, 2H), 7.22 (d, J=2.4 Hz, 1H), 7.05-6.98 (m, 1H), 6.88 (d, J=8.6 Hz, 1H), 6.76 (d, J=8.8 Hz, 1H), 6.24 (t, J=2.4 Hz, 1H), 5.25-5.15 (m, 1H), 5.07 (s, 1H), 4.36-4.24 (m, 3H), 4.04 (d, J=8.8 Hz, 1H), 3.67-3.58 (m, 2H), 3.55 (s, 3H), 2.08-1.96 (m, 2H), 1.67-1.55 (m, 2H), 1.47 (s, 6H), 1.22 (s, 6H), 1.12 (s, 6H). LC-MS: MS (ES⁺): RT=2.730 min, m/z=857.4[M+H⁺], LCMS method: 25.

### Example 54—Synthesis of N-[3-(3-chloro-4-cyano-phenoxy)-2,2,4,4-tetramethyl-cyclobutyl]-2-[4-[[5-[4-(1-hydroxy-1-methyl-ethyl)-2-(6-methyl-7-oxo-1H-pyrrolo[2,3-c]pyridin-4-yl)phenoxy]-2-pyridyl]methyl]-1-piperidyl]pyrimidine-5-carboxamide (I-298)

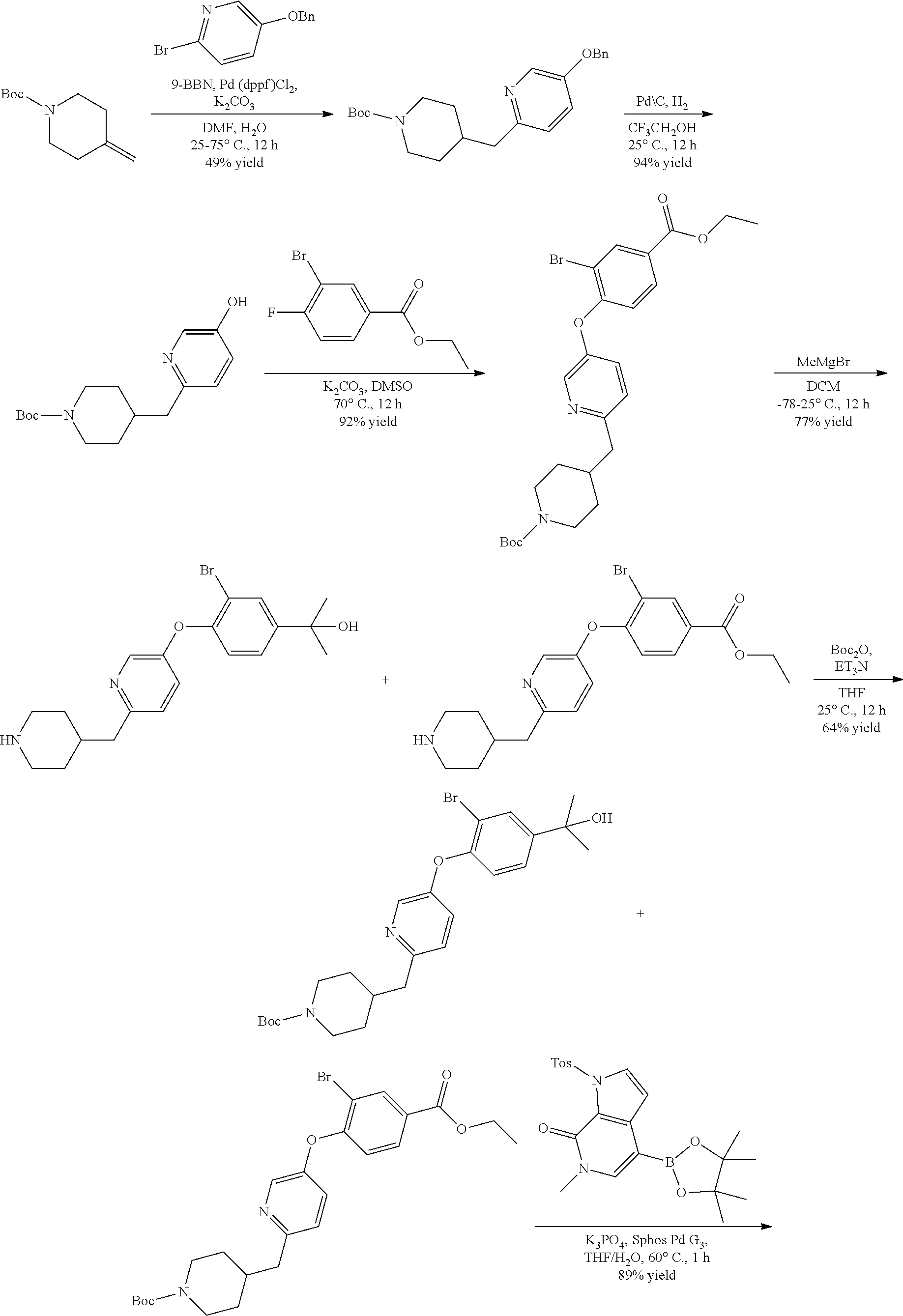

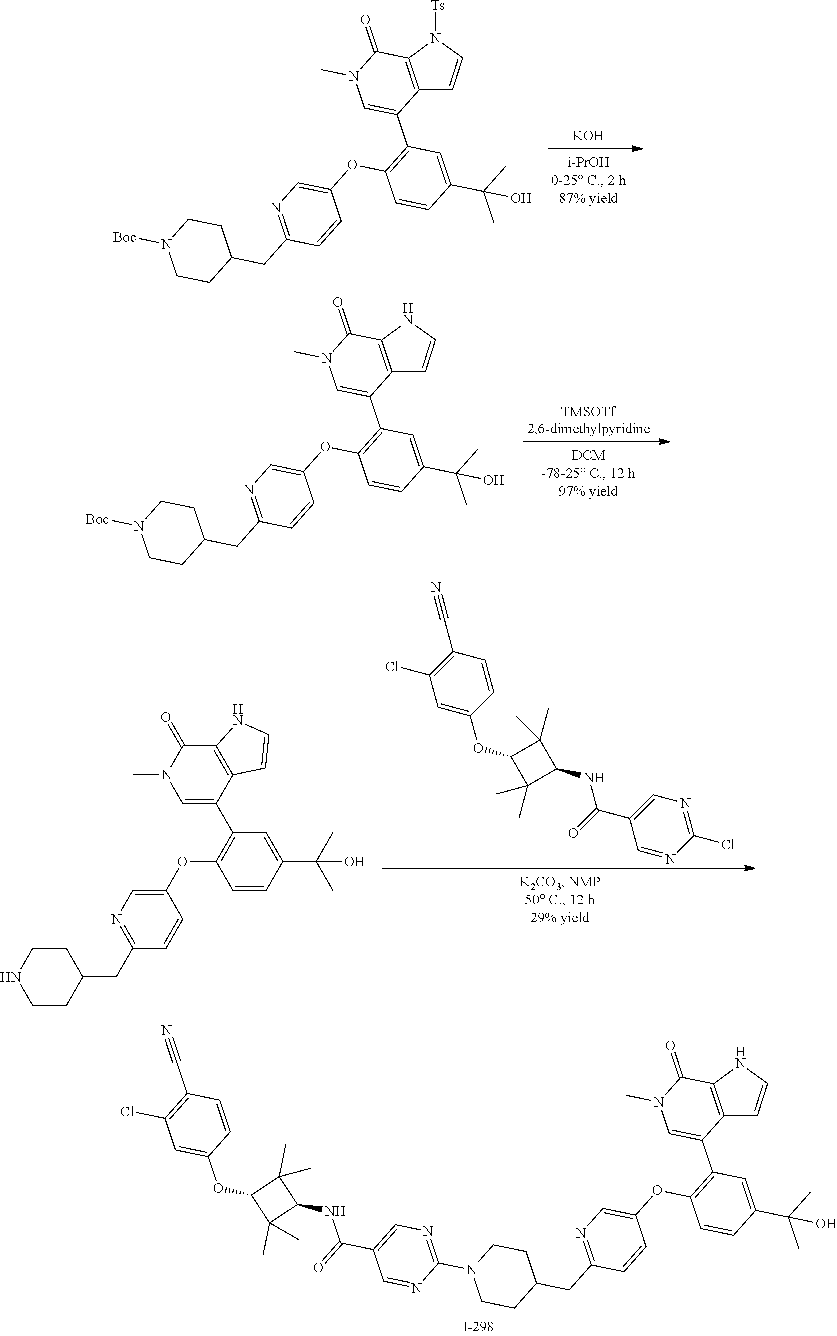 [SMILES: Cc1ccc(S(=O)(=O)n2ccc3c(-c4cc(C(C)(C)O)ccc4Oc4ccc(CC5CCN(C(=O)OC(C)(C)C)CC5)nc4)cn(C)c(=O)c32)cc1 ; "KOH" ; Cn1cc(-c2cc(C(C)(C)O)ccc2Oc2ccc(CC3CCN(C(=O)OC(C)(C)C)CC3)nc2)c2cc[nH]c2c1=O ; Cn1cc(-c2cc(C(C)(C)O)ccc2Oc2ccc(CC3CCNCC3)nc2)c2cc[nH]c2c1=O ; CC1(C)[C@H](NC(=O)c2cnc(Cl)nc2)C(C)(C)[C@H]1Oc1ccc(C#N)c(Cl)c1 ; Cn1cc(-c2cc(C(C)(C)O)ccc2Oc2ccc(CC3CCN(c4ncc(C(=O)N[C@H]5C(C)(C)[C@H](Oc6ccc(C#N)c(Cl)c6)C5(C)C)cn4)CC3)nc2)c2cc[nH]c2c1=O]

Step 1: Preparation of tert-butyl 4-[(5-benzyloxy-2-pyridyl)methyl]piperidine-1-carboxylate. A mixture of tert-butyl 4-methylenepiperidine-1-carboxylate (5 g, 25.35 mmol, 1.0 equiv) and 9-BBN (0.5 M, 50.7 mL, 1.0 equiv) was stirred at 70° C. for 1 h. The reaction mixture was cooled to 25° C. and added to a solution of 5-benzyloxy-2-bromo-pyridine (10.0 g, 38.0 mmol, 1.5 equiv), cyclopentyl(diphenyl)phosphane;dichloromethane;dichloropalladium;iron (2.07 g, 2.53 mmol, 0.10 equiv), and K₂CO₃ (8.76 g, 63.4 mmol, 2.5 equiv) dissolved in DMF (50 mL) and H₂O (10 mL). This mixture was heated at 60° C. for 12 h. The mixture was poured into ice-water (200 mL). The aqueous phase was extracted with ethyl acetate (80 mL×3). The combined organic phase was washed with brine (100 mL×2), dried with anhydrous sodium sulfate, filtered and concentrated under vacuum. The residue was purified by silica gel column chromatography (petroleum ether: ethyl acetate=5:1 to 1:1). Tert-butyl 4-[(5-benzyloxy-2-pyridyl)methyl]piperidine-1-carboxylate (5 g, 12.4 mmol, 49% yield) was obtained as an off-white solid.

Step 2: Preparation of tert-butyl 4-[(5-hydroxy-2-pyridyl)methyl]piperidine-1-carboxylate. To a solution of tert-butyl 4-[(5-benzyloxy-2-pyridyl)methyl]piperidine-1-carboxylate (5.00 g, 13.1 mmol, 1.0 equiv) in CF₃CH₂OH (80 mL) was added Pd/C (2.50 g, 2.35 mmol, 10% purity, 0.18 equiv) under N₂ atmosphere. The suspension was degassed and purged with H₂ three times. The mixture was stirred under H₂ (15 psi) at 25° C. for 12 h. The reaction mixture was filtered and the filter cake was washed with ethanol (20 mL×2). The collected filtrate was concentrated to give a residue. The residue was used for the next step without further purification. Tert-butyl 4-[(5-hydroxy-2-pyridyl)methyl]piperidine-1-carboxylate (3.6 g, 12.3 mmol, 94% yield) was obtained as a white solid.

Step 3: Preparation of tert-butyl 4-[[5-(2-bromo-4-ethoxycarbonyl-phenoxy)-2-pyridyl]methyl]piperidine-1-carboxylate. To a solution of tert-butyl 4-[(5-hydroxy-2-pyridyl)methyl]piperidine-1-carboxylate (1.20 g, 4.10 mmol, 1.0 equiv) and ethyl 3-bromo-4-fluoro-benzoate (1.22 g, 4.93 mmol, 1.2 equiv) in DMSO (10 mL) was added K₂CO₃ (1.70 g, 12.3 mmol, 3.0 equiv) and stirred at 70° C. for 12 h. The reaction mixture was filtered and washed with ethanol (20 mL×2). The collected filtrate was concentrated to give a residue. The residue was used for the next step without further purification. Tert-butyl 4-[[5-(2-bromo-4-ethoxycarbonyl-phenoxy)-2-pyridyl]methyl]piperidine-1-carboxylate (2.00 g, 3.77 mmol, 92% yield) was obtained as a white solid.

Step 4: Preparation of ethyl 3-bromo-4-[16-(4-piperidylmethyl)-3-pyridyl]oxy]benzoate. To a mixture of methyltert-butyl 4-[[5-(2-bromo-4-ethoxycarbonyl-phenoxy)-2-pyridyl]methyl]piperidine-1-carboxylate (1.00 g, 1.93 mmol, 1.0 equiv) in DCM (10 mL) was added MeMgBr (3 M, 9.63 mL, 15.0 equiv) at −78° C. and the mixture stirred at 25° C. for 12 h. The reaction mixture was quenched with saturated aqueous NH₄Cl (10 mL) solution at 0° C. by dropwise addition and diluted with water (15 mL). And the resulting mixture was extracted with DCM (40 mL×3). The combined organic layers were dried over anhydrous Na₂SO₄, filtered and concentrated in vacuum. The residue was used for the next step without further purification. 2-[3-bromo-4-[[6-(4-piperidylmethyl)-3-pyridyl]oxy]phenyl]propan-2-ol (0.600 g, 1.48 mmol, 77% yield) was obtained as a white solid. Ethyl 3-bromo-4-[[6-(4-piperidylmethyl)-3-pyridyl]oxy]benzoate (0.08 g, 191 μmol, 10% yield) was obtained as a white solid.

Step 5: Preparation of tert-butyl 4-[[5-(2-bromo-4-ethoxycarbonyl-phenoxy)-2-pyridyl]methyl]piperidine-1-carboxylate. To a mixture of methyl2-[3-bromo-4-[[6-(4-piperidylmethyl)-3-pyridyl]oxy]phenyl]propan-2-ol (0.500 g, 1.23 mmol, 1.0 equiv) and ethyl 3-bromo-4-[[6-(4-piperidylmethyl)-3-pyridyl]oxy]benzoate (0.08 g, 191 mol, 0.16 equiv) in THF (7 mL) was added TEA (500 mg, 4.93 mmol, 687 μL, 4.0 equiv) and Boc₂₀ (808 mg, 3.70 mmol, 850 μL, 3.0 equiv) and stirred at 25° C. for 12 h. The mixture was poured into ice-water (20 mL). The aqueous phase was extracted with ethyl acetate (20 mL×3). The combined organic phase was washed with brine (10 mL×2), dried with anhydrous sodium sulfate, filtered and concentrated under vacuum. The residue was purified by silica gel column chromatography (petroleum ether: ethyl acetate=1:1 to 0:1). Tert-butyl 4-[[5-[2-bromo-4-(1-hydroxy-1-methyl-ethyl)phenoxy]-2-pyridyl]methyl]piperidine-1-carboxylate (0.400 g, 791 mol, 64% yield) was obtained as a white solid. Tert-butyl 4-[[5-(2-bromo-4-ethoxycarbonyl-phenoxy)-2-pyridyl]methyl]piperidine-1-carboxylate (0.120 g, 231 μmol, 19% yield) was obtained as a white solid.

Step 6: Preparation of tert-butyl 4-[[5-[4-(1-hydroxy-1-methyl-ethyl)-2-[6-methyl-7-oxo-1-(p-tolylsulfonyl)pyrrolo[2,3-c]pyridin-4-yl]phenoxy]-2-pyridyl]methyl]piperidine-1-carboxylate. To a mixture of methyltert-butyl 4-[[5-[2-bromo-4-(1-hydroxy-1-methyl-ethyl)phenoxy]-2-pyridyl]methyl]piperidine-1-carboxylate (0.350 g, 692 μmol, 1.0 equiv), 6-methyl-1-(p-tolylsulfonyl)-4-(4,4,5,5-tetramethyl-1,3,2-dioxaborolan-2-yl)pyrrolo[2,3-c]pyridin-7-one (386 mg, 900 μmol, 1.3 equiv) and SPhos Pd G₃ (108 mg, 138 μmol, 0.2 equiv) in THF (2 mL) was added a solution of K₃PO₄ (441 mg, 2.08 mmol, 3.0 equiv) in H₂O(0.5 mL) and stirred at 60° C. for 1 h. The mixture was diluted with EA (40 mL), poured into ice-water (20 mL). The aqueous phase was extracted with ethyl acetate (20 mL×3). The combined organic phase was washed with brine (10 mL×2), dried with anhydrous sodium sulfate, filtered and concentrated under vacuum. The residue was purified by prep-TLC (petroleum ether: ethyl acetate=0:1). Tert-butyl 4-[[5-[4-(1-hydroxy-1-methyl-ethyl)-2-[6-methyl-7-oxo-1-(p-tolylsulfonyl)pyrrolo[2,3-c]pyridin-4-yl]phenoxy]-2-pyridyl]methyl]piperidine-1-carboxylate (0.450 g, 619 μmol, 89% yield) was obtained as a white solid.

Step 7: Preparation of tert-butyl 4-[[5-[4-(1-hydroxy-1-methyl-ethyl)-2-(6-methyl-7-oxo-1H-pyrrolo[2,3-c]pyridin-4-yl)phenoxy]-2-pyridyl]methyl]piperidine-1-carboxylate. To a mixture of methyl tert-butyl 4-[[5-[4-(1-hydroxy-1-methyl-ethyl)-2-[6-methyl-7-oxo-1-(p-tolylsulfonyl)pyrrolo[2,3-c]pyridin-4-yl]phenoxy]-2-pyridyl]methyl]piperidine-1-carboxylate (0.450 g, 619 mol, 1.0 equiv) in i-PrOH (4 mL) was added KOH (1 M, 9.29 mL, 15.0 equiv) and stirred at 25° C. for 2 h. The mixture was diluted with EA (30 mL), poured into ice-water (20 mL). The aqueous phase was extracted with ethyl acetate (20 mL×3). The combined organic phase was washed with brine (15 mL×2), dried with anhydrous sodium sulfate, filtered and concentrated under vacuum. The residue was purified by prep-TLC (petroleum ether: ethyl acetate=0:1). Tert-butyl 4-[[5-[4-(1-hydroxy-1-methyl-ethyl)-2-(6-methyl-7-oxo-1H-pyrrolo[2,3-c]pyridin-4-yl)phenoxy]-2-pyridyl]methyl]piperidine-1-carboxylate (0.330 g, 536 μmol, 87% yield) was obtained as a white solid.

Step 8: Preparation of 4-[5-(1-hydroxy-1-methyl-ethyl)-2-[[6-(4-piperidyl methyl)-3-pyridyl]oxy]phenyl]-6-methyl-1H-pyrrolo[2,3-c]pyridin-7-one. To a mixture of methyl tert-butyl 4-[[5-[4-(1-hydroxy-1-methyl-ethyl)-2-(6-methyl-7-oxo-1H-pyrrolo[2,3-c]pyridin-4-yl)phenoxy]-2-pyridyl]methyl]piperidine-1-carboxylate (0.250 g, 437 μmol, 1.0 equiv) and 2,6-dimethylpyridine (702 mg, 6.55 mmol, 763 μL, 15.0 equiv) in DCM (2 mL) was added TMSOTf (970 mg, 4.37 mmol, 789 μL, 10.0 equiv) at −78° C. and stirred at 25° C. for 12 h. The reaction mixture was quenched with water (1 mL) at −78° C. by dropwise addition. And the resulting mixture was extracted with ethyl acetate (40 mL×3). The combined organic layers were dried over anhydrous Na₂SO₄, filtered and concentrated in vacuum. The residue was used for the next step without further purification. 4-[5-(1-hydroxy-1-methyl-ethyl)-2-[[6-(4-piperidylmethyl)-3-pyridyl]oxy]phenyl]-6-methyl-1H-pyrrolo[2,3-c]pyridin-7-one (0.200 g, 423 μmol, 97% yield) was obtained as a light yellow solid.

Step 9: Preparation of N-[3-(3-chloro-4-cyano-phenoxy)-2,2,4,4-tetramethyl-cyclobutyl]-2-[4-[[5-[4-(1-hydroxy-1-methyl-ethyl)-2-(6-methyl-7-oxo-1H-pyrrolo[2,3-c]pyridin-4-yl)phenoxy]-2-pyridyl]methyl]-1-piperidyl]pyrimidine-5-carboxamide (I-298). To a mixture of methyl 4-[5-(1-hydroxy-1-methyl-ethyl)-2-[[6-(4-piperidylmethyl)-3-pyridyl]oxy]phenyl]-6-methyl-¹H-pyrrolo[2,3-c]pyridin-7-one (80.0 mg, 169 μmol, 1.0 equiv) and 2-chloro-N-[3-(3-chloro-4-cyano-phenoxy)-2,2,4,4-tetramethyl-cyclobutyl]pyrimidine-5-carboxamide (71.0 mg, 169 mol, 1.0 equiv) in NMP (2 mL) was added K₂CO₃(70.2 mg, 508 μmol, 3.0 equiv) and stirred at 50° C. for 12 h. The mixture was poured into ice-water (20 mL). The aqueous phase was extracted with dichloromethane (20 mL×3). The combined organic phase was washed with brine (10 mL×2), dried with anhydrous sodium sulfate, filtered and concentrated under vacuum. The residue was purified by semi-preparative reverse phase HPLC (column: Phenomenex Luna C18 150 mm*25 mm*10 μm; mobile phase: [water(FA)-ACN]; B %: 50%-80%, 10 min). N-[3-(3-chloro-4-cyano-phenoxy)-2,2,4,4-tetramethyl-cyclobutyl]-2-[4-[[5-[4-(1-hydroxy-1-methyl-ethyl)-2-(6-methyl-7-oxo-1H-pyrrolo[2,3-c]pyridin-4-yl)phenoxy]-2-pyridyl]methyl]-1-piperidyl]pyrimidine-5-carboxamide (44.35 mg, 49.77 μmol, 29% yield, 96% purity) was obtained as a white solid.

¹H NMR (400 MHz, DMSO-d₆): δ=12.01 (s, 1H) 8.74 (s, 2H) 8.09 (d, J=2.8 Hz, 1H) 7.90 (d, J=8.8 Hz, 1H) 7.70 (d, J=9.2 Hz, 1H) 7.60 (d, J=2.0 Hz, 1H) 7.48 (m, 1H) 7.27 (t, J=2.8 Hz, 1H) 7.19-7.24 (m, 3H) 7.14 (d, J=8.8 Hz, 1H) 6.98-7.05 (m, 2H) 6.21 (t, J=2.4 Hz, 1H) 4.70 (d, J=13.2 Hz, 2H) 4.28 (s, 1H) 4.03 (d, J=9.2 Hz, 1H) 3.51 (s, 3H) 2.90 (t, J=11.6 Hz, 2H) 2.59 (d, J=7.2 Hz, 2H) 2.30-2.35 (m, 3H) 1.94-2.02 (m, 1H) 1.53-1.61 (m, 2H) 1.48 (s, 6H) 1.21 (s, 6H) 1.11 (s, 6H).LC-MS: MS (ES⁺): RT=2.020 min, m/z=855.4 [M+H⁺] LCMS method: 25.

### Example 55—Synthesis of N-[3-(3-chloro-4-cyano-phenoxy)-2,2,4,4-tetramethyl-cyclobutyl]-2-[4-[[3-[4-(1-hydroxy-1-methyl-ethyl)-2-(6-methyl-7-oxo-1H-pyrrolo[2,3-c]pyridin-4-yl)phenoxy]cyclobutyl]methyl]-1-piperidyl]pyrimidine-5-carboxamide (I-309)

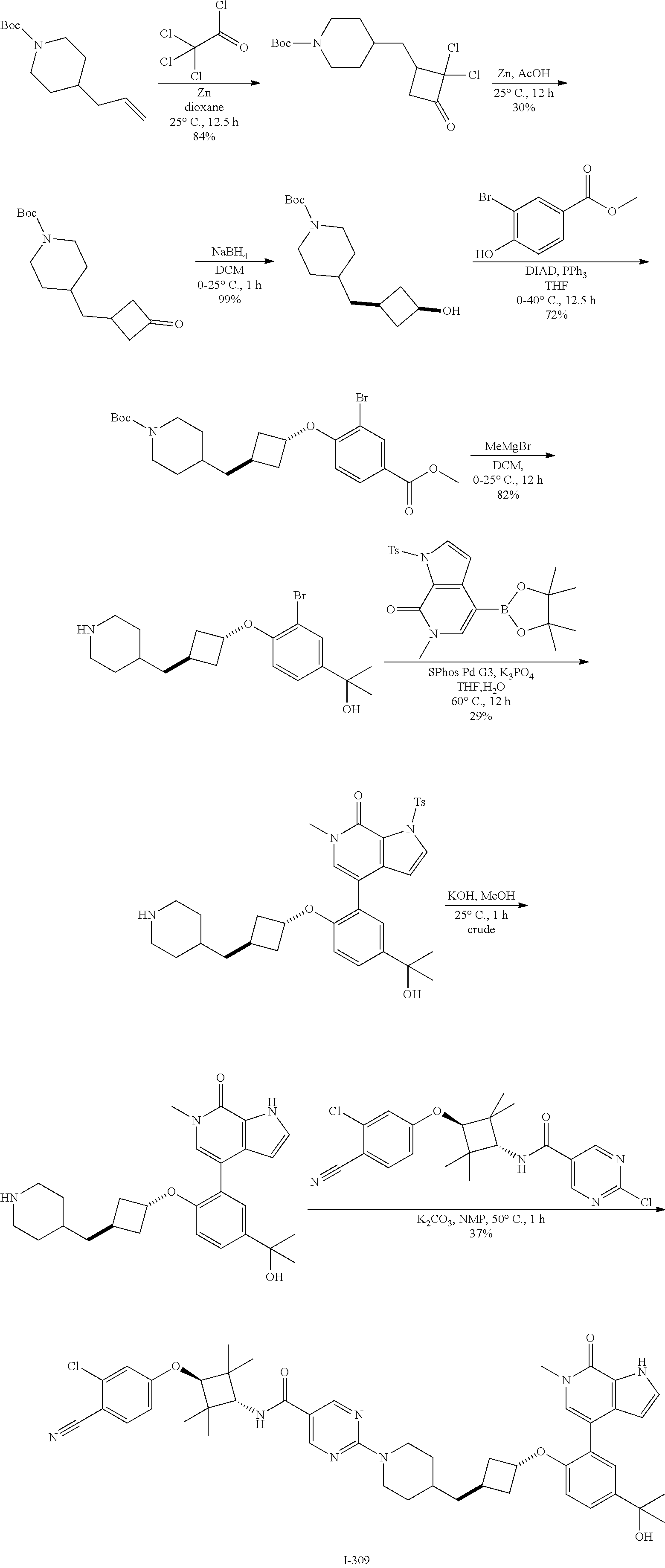

Step 1: Preparation of tert-butyl 4-[(2,2-dichloro-3-oxo-cyclobutyl)methyl]piperidine-1-carboxylate. To a solution of tert-butyl 4-allylpiperidine-1-carboxylate (8.4 g, 37 mmol, 1.0 equiv) and Zn (12 g, 186 mmol, 5.0 equiv) in dioxane (110 mL) was slowly dropwise added a solution of 1,2-dimethoxyethane (10 g, 111 mmol, 12 mL, 3.0 equiv) and 2,2,2-trichloroacetyl chloride (20 g, 111 mmol, 12 mL, 3.0 equiv) in dioxane (110 mL) over a period of about 0.5 h under N₂. The mixture was stirred at 25° C. for 12 h. The mixture was filtered and concentrated. The residue was purified by column chromatography (SiO₂, petroleum ether: ethyl acetate=20:1 to 10:1) to give tert-butyl 4-[(2,2-dichloro-3-oxo-cyclobutyl)methyl]piperidine-1-carboxylate (11 g, 84% yield) as a yellow oil.

Step 2: Preparation of tert-butyl 4-[(3-oxocyclobutyl)methyl]piperidine-1-carboxylate. To a solution of tert-butyl 4-[(2,2-dichloro-3-oxo-cyclobutyl)methyl]piperidine-1-carboxylate (5 g, 15 mmol, 1.0 equiv) in AcOH (50 mL) was added Zn (4 g, 61 mmol, 4.0 equiv). The mixture was stirred at 25° C. for 3 h. The reaction mixture was filtered and concentrated. The residue was purified by column chromatography (SiO₂, petroleum ether: ethyl acetate=1:0 to 20:1) to give tert-butyl 4-[(3-oxocyclobutyl)methyl]piperidine-1-carboxylate (1.2 g, 30% yield).

Step 3: Preparation of tert-butyl 4-[(3-hydroxycyclobutyl)methyl]piperidine-1-carboxylate. To a solution of tert-butyl 4-[(3-oxocyclobutyl)methyl]piperidine-1-carboxylate (500 mg, 2.0 mmol, 1.0 equiv) in DCM (10 mL) was added NaBH₄ (200 mg, 5.3 mmol, 2.8 equiv) at 0° C. The mixture was stirred at 25° C. for 1 h and concentrated. The residue was purified by column chromatography (SiO₂, petroleum ether: ethyl acetate=1:0 to 3:1) to give tert-butyl 4-[(3-hydroxycyclobutyl)methyl]piperidine-1-carboxylate (500 mg, 99% yield).

Step 4: Preparation of tert-butyl 4-[[3-(2-bromo-4-methoxycarbonyl-phenoxy) cyclobutyl]methyl]piperidine-1-carboxylate. To a solution of PPh₃ (5 g, 19 mmol, 7.0 equiv) in THF (40 mL) was added DIAD (3.9 g, 19 mmol, 3.7 mL, 7.0 equiv) at 0° C. and the solution was stirred at 25° C. for 0.5 h. Then 16.5 mL of the mixture was added to the solution of tert-butyl 4-[(3-hydroxycyclobutyl)methyl]piperidine-1-carboxylate (700 mg, 2.6 mmol, 1.0 equiv) and methyl 3-bromo-4-hydroxy-benzoate (1.2 g, 5 mmol, 2.0 equiv) in THF (5 mL) at 25° C. The mixture was stirred at 40° C. for 12 h and concentrated. The residue was purified by prep-HPLC (column: Phenomenex luna Cis 150 m*40 mm*15 μm; mobile phase: [water(FA)-ACN]; B %: 70%-100%, 15 min) to give tert-butyl 4-[[3-(2-bromo-4-methoxycarbonyl-phenoxy)cyclobutyl]methyl]piperidine-1-carboxylate (900 mg, 72% yield).

Step 5: Preparation of 2-[3-bromo-4-13-(4-piperidylmethyl)cyclobutoxy]phenyl]propan-2-ol. To a solution of tert-butyl 4-[[3-(2-bromo-4-methoxycarbonyl-phenoxy)cyclobutyl]methyl]piperidine-1-carboxylate (200 mg, 415 μmol, 1.0 equiv) in DCM (5 mL) was added MeMgBr (3 M, 3 mL, 20.0 equiv) at 0° C. The mixture was stirred at 25° C. for 12 h. The reaction mixture was quenched by saturated aqueous NH₄Cl solution (5 mL) at 0° C. And the resulting mixture was extracted with EA (30 mL*3). The combined organic layers were dried over anhydrous Na₂SO₄ and concentrated. The residue was purified by prep-HPLC (column: UniSil 3-100 C18 Ultra (150 mm*25 mm*3 ™); mobile phase: [water(FA)-ACN]; B %: 20%-40%, 7 min) to give 2-[3-bromo-4-[3-(4-piperidylmethyl) cyclobutoxy]phenyl]propan-2-ol (130 mg, 82% yield).

Step 6: Preparation of 4-[5-(1-hydroxy-1-methyl-ethyl)-2-[3-(4-piperidyl methyl)cyclobutoxy]phenyl]-6-methyl-1-(p-tolylsulfonyl)pyrrolo[2,3-c]pyridin-7-one. A mixture of 2-[3-bromo-4-[3-(4-piperidylmethyl)cyclobutoxy]phenyl]propan-2-ol (130 mg, 340 μmol, 1.0 equiv), 6-methyl-1-(p-tolylsulfonyl)-4-(4,4,5,5-tetramethyl-1,3,2-dioxaborolan-2-yl)pyrrolo[2,3-c]pyridin-7-one (175 mg, 408 μmol, 1.2 equiv), K₃PO₄ (217 mg, 1 mmol, 3.0 equiv), SPhos Pd G3 (27 mg. 34 mol, 0.1 equiv) in THF (5 mL) and H₂O (1 mL) was degassed and purged with N₂ three times. The mixture was stirred at 60° C. for 12 h under an N₂ atmosphere and concentrated. The residue was purified by prep-HPLC (column: Phenomenex luna C₁₋₈ 150 mm*25 mm*10 μm; mobile phase: [water(FA)-ACN]; B %: 16%-46%, 10 min) to give 4-[5-(1-hydroxy-1-methyl-ethyl)-2-[3-(4-piperidylmethyl) cyclobutoxy]phenyl]-6-methyl-1-(p-tolylsulfonyl)pyrrolo[2,3-c]pyridin-7-one (60 mg, 29% yield) as a white solid.

Step 7: Preparation of 4-[5-(1-hydroxy-1-methyl-ethyl)-2-[3-(4-piperidyl-methyl)cyclobutoxy]phenyl]-6-methyl-1H-pyrrolo[2,3-c]pyridin-7-one. To a solution of 4-[5-(1-hydroxy-1-methyl-ethyl)-2-[3-(4-piperidylmethyl)cyclobutoxy]phenyl]-6-methyl-1-(p-tolylsulfonyl)pyrrolo[2,3-c]pyridin-7-one (60 mg, 99 μmol, 1.0 equiv) in MeOH (0.5 mL) was added KOH (56 mg, 994 mol, 10.0 equiv). The mixture was stirred at 25° C. for 1 h. The residue was diluted with H₂O and extracted with DCM (15 mL*4). The combined organic layers were dried over anhydrous Na₂SO₄ and concentrated under reduced pressure to give a crude product.

Step 8: Preparation of N-[3-(3-chloro-4-cyano-phenoxy)-2,2,4,4-tetramethyl-cyclobutyl]-2-[4-[[3-[4-(1-hydroxy-1-methyl-ethyl)-2-(6-methyl-7-oxo-1H-pyrrolo[2,3-c]pyridin-4-yl)phenoxy]cyclobutyl]methyl]-1-piperidyl]pyrimidine-5-carboxamide (I-309). To a solution of 4-[5-(1-hydroxy-1-methyl-ethyl)-2-[3-(4-piperidylmethyl)cyclobutoxy]phenyl]-6-methyl-1H-pyrrolo[2,3-c]pyridin-7-one (100 mg, 222 mol, 1.0 equiv) in NMP (2 mL) was added DIEA (144 mg, 1.11 mmol, 194 μL, 5.0 equiv) and 2-chloro-N-[3-(3-chloro-4-cyano-phenoxy)-2,2,4,4-tetramethyl-cyclobutyl]pyrimidine-5-carboxamide (93 mg, 0.22 mmol, 1.0 equiv). The mixture was stirred at 50° C. for 1 h. The reaction was filtered and the filtrate was concentrated in vacuo to give the crude product. The crude product was purified by preparative HPLC (column: Phenomenex Luna C18 150 mm*25 mm*10 μm; mobile phase: [water(FA)-ACN]; gradient: 64%-94% B over 10 min) to yield N-[3-(3-chloro-4-cyano-phenoxy)-2,2,4,4-tetramethyl-cyclobutyl]-2-[4-[[3-[4-(1-hydroxy-1-methyl-ethyl)-2-(6-methyl-7-oxo-1H-pyrrolo[2,3-c]pyridin-4-yl)phenoxy]cyclobutyl]methyl]-1-piperidyl]pyrimidine-5-carboxamide (70 mg, 0.82 mmol, 37% yield, 98% purity) was obtained as a yellow solid. ¹H NMR (400 MHz, DMSO-d₆) 311.96 (s, 1H), 8.74 (s, 2H) 7.90 (d, J=8.8 Hz, 1H), 7.70 (d, J=9.6 Hz, 1H), 7.43 (d, J=2.4 Hz, 1H), 7.34 (s, 1H), 7.27 (t, J=2.8 Hz, 1H), 7.23-7.15 (m, 2H), 7.00 (s, 1H), 6.77 (d, J=8.8 Hz, 1H), 6.17-6.06 (m, 1H), 4.92 (s, 1H), 4.82-4.66 (m, 3H), 4.28 (s, 1H), 4.03 (d, J=9.2 Hz, 1H), 3.62-3.52 (m, 3H), 2.97-2.85 (m, 2H), 2.57-2.51 (m, 2H), 2.39-2.29 (m, 1H), 2.08 (t, J=6.0 Hz, 3H), 1.69 (d, J=11.6 Hz, 2H), 1.60-1.50 (m, 1H), 1.43 (s, 6H), 1.40-1.37 (m, 1H), 1.21 (s, 6H), 1.10 (s, 6H), 1.06-0.96 (m, 2H).LC-MS: MS (ES⁺): RT=2.932 min, m/z=832.4 [M+H⁺]; LCMS method: 25.

### Example 56—Synthesis of N-[3-(3-chloro-4-cyano-phenoxy)-2,2,4,4-tetramethyl-cyclobutyl]-2-[3-[[1-[4-[1-hydroxy-1-methyl-6-(6-methyl-7-oxo-1H-pyrrolo[2,3-c]pyridin-4-yl)indan-5-yl]oxyphenyl]azetidin-3-yl]methyl]azetidin-1-yl]pyrimidine-5-carboxamide (I-329)

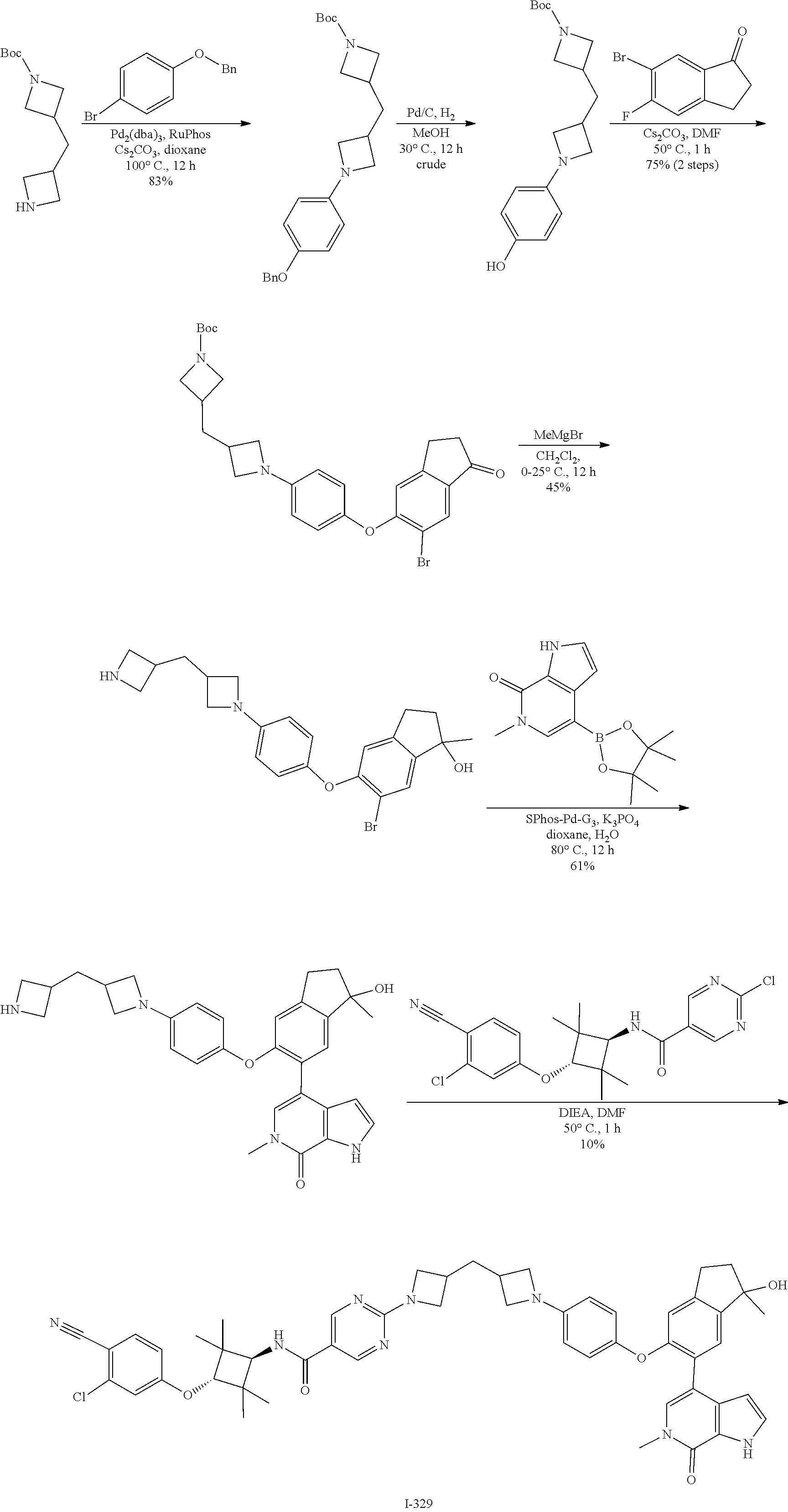

Step 1: Preparation of tert-butyl 3-[[1-(4-benzyloxyphenyl)azetiidin-3-yl]methyl]azetidine-1-carboxylate. A mixture of tert-butyl 3-(azetidin-3-ylmethyl) azetidine-1-carboxyl ate (2.00 g, 8.84 mmol, 1 equiv), 1-benzyloxy-4-bromo-benzene (2.56 g, 9.72 mmol, 1.1 equiv), Pd₂(dba)₃ (809 mg, 0.88 mmol, 0.1 equiv), Cs₂CO₃ (8.64 g, 26.5 mmol, 3 equiv) and RuPhos (825 mg, 1.77 mmol, 0.2 equiv) in dioxane (30 mL) was stirred at 100° C. for 12 h under N₂ atmosphere. The reaction mixture was filtered and concentrated in vacuo. The residue was purified by column chromatography on silica gel (Petroleum ether/Ethyl acetate=5/1 to 3/1) to afford tert-butyl 3-[[1-(4-benzyloxyphenyl) azetidin-3-yl]methyl]azetidine-1-carboxylate (3.00 g, 7.34 mmol, 83% yield) as a yellow solid.

Step 2: Preparation of tert-butyl 3-[[1-(4-hydroxyphenyl)azetidin-3-yl]methyl]azetidine-1-carboxylate. To a solution of tert-butyl 3-[[1-(4-benzyloxyphenyl)azetidin-3-yl]methyl]azetidine-1-carboxylate (2.90 g, 7.10 mmol, 1 equiv) in MeOH (40 mL) was added Pd/C (1.00 g, 10% purity) at 20° C. under N₂ atmosphere. The suspension was stirred under H₂ (15 psi) at 30° C. for 12 h. The reaction mixture was filtered and concentrated in vacuo to afford tert-butyl 3-[[1-(4-hydroxyphenyl)azetidin-3-yl]methyl]azetidine-1-carboxylate (2.11 g, crude) as a colorless oil.

Step 3: Preparation of tert-butyl 3-[[1-[4-(6-bromo-1-oxo-indan-5-yl)oxyphenyl]azetidin-3-yl]methyl]azetidine-1-carboxylate. To a solution of tert-butyl 3-[[1-(4-hydroxyphenyl)azetidin-3-yl]methyl]azetidine-1-carboxylate (1.20 g, 3.77 mmol, 1 equiv) and 6-bromo-5-fluoro-indan-1-one (0.95 g, 4.15 mmol, 1.1 equiv) in DMF (20 mL) was added Cs₂CO₃ (3.68 g, 11.3 mmol, 3 equiv) at 20° C. The mixture was stirred at 50° C. for 1 h. The reaction mixture was diluted with H₂O (50 mL) and extracted with ethyl acetate (30 mL*3). The combined organic phase was washed with brine (20 mL*3), dried with anhydrous Na₂SO₄, filtered and concentrated in vacuo. The residue was purified by column chromatography on silica gel (Petroleum ether/Ethyl acetate=3/1 to 1/1) to afford tert-butyl 3-[[1-[4-(6-bromo-1-oxo-indan-5-yl)oxyphenyl]azetidin-3-yl]methyl]azetidine-1-carboxylate (1.60 g, 3.03 mmol, 75% yield, 2 steps) as a yellow solid.

Step 4: Preparation of 5-[4-[3-(azetidin-3-ylmethyl)azetidin-1-yl]phenoxy]-6-bromo-1-methyl-indan-1-ol. To a solution of tert-butyl 3-[[1-[4-(6-bromo-1-oxo-indan-5-yl)oxyphenyl]azetidin-3-yl]methyl]azetidine-1-carboxylate (1.20 g, 2.28 mmol, 1 equiv) in CH₂Cl₂ (20 mL) was added dropwise MeMgBr (3 M, 11.4 mL, 15 equiv) at 0° C. The resulting mixture was stirred at 25° C. for 12 h. The reaction mixture was poured into water (50 mL) and extracted with CH₂Cl₂ (30 mL*3). The combined organic phase was washed with brine (30 mL*3), dried with anhydrous Na₂SO₄, filtered and concentrated in vacuo. The residue was purified by column chromatography on silica gel (Dichloromethane/Methanol=50/1 to 20/1) to afford 5-[4-[3-(azetidin-3-ylmethyl)azetidin-1-yl]phenoxy]-6-bromo-1-methyl-indan-1-ol (0.45 g, 1.01 mmol, 45% yield) as a yellow solid.

Step 5: Preparation of 4-[6-[4-[3-(azetidin-3-ylmethyl)azetidin-1-yl]phenoxy]-3-hydroxy-3-methyl-indan-5-yl]-6-methyl-1Hpyrrolo[2,3-c]pyridin-7-one. A mixture of 5-[4-[3-(azetidin-3-ylmethyl)azetidin-1-yl]phenoxy]-6-bromo-1-methyl-indan-1-ol (100 mg, 0.23 mmol, 1 equiv), 6-methyl-4-(4,4,5,5-tetramethyl-1,3,2-dioxaborolan-2-yl)-1H-pyrrolo[2,3-c]pyridin-7-one (74.2 mg, 0.27 mmol, 1.2 equiv), SPhos Pd G3 (17.6 mg, 0.02 mmol, 0.1 equiv) and K₃PO₄ (144 mg, 0.68 mmol, 3 equiv) in dioxane (4 mL) and H₂O (0.5 mL) was stirred at 80° C. for 12 h under N₂ atmosphere. The reaction mixture was concentrated in vacuo. The residue was purified by column chromatography on silica gel (Dichloromethane/Methanol=20/1 to 10/1) to afford 4-[6-[4-[3-(azetidin-3-ylmethyl) azetidin-1-yl]phenoxy]-3-hydroxy-3-methyl-indan-5-yl]-6-methyl-1Hpyrrolo[2,3-c]pyridin-7-one (70.0 mg, 0.14 mmol, 61% yield) as a yellow solid.

Step 6: Preparation of N-[3-(3-chloro-4-cyano-phenoxy)-2,2,4,4-tetramethyl-cyclobutyl]-2-[3-[[1-[4-[1-hydroxy-1-methyl-6-(6-methyl-7-oxo-1H-pyrrolo[2,3-c]pyridin-4-yl)indan-5-yl]oxyphenyl]azetidin-3-yl]methyl]azetidin-1-yl]pyrimidine-5-carboxamide. To a solution of 4-[6-[4-[3-(azetidin-3-ylmethyl)azetidin-1-yl]phenoxy]-3-hydroxy-3-methyl-indan-5-yl]-6-methyl]H-pyrrolo[2,3-c]pyridin-7-one (70.0 mg, 0.14 mmol, 1 equiv) and 2-chloro-N-[3-(3-chloro-4-cyano-phenoxy)-2,2,4,4-tetramethyl-cyclobutyl]pyrimidine-5-carboxamide (57.5 mg, 0.14 mmol, 1 equiv) in DMF (2 mL) was added DIPEA (53.2 mg, 0.41 mmol, 3 equiv). The mixture was stirred at 50° C. for 1 h. The reaction mixture was filtered and the filtrate was purified by prep-HPLC (NH₄HCO₃ as additive) to give crude product, and further by prep-TLC on silica gel (Dichloromethane/Methanol=10/1) to afford N-[3-(3-chloro-4-cyano-phenoxy)-2,2,4,4-tetramethyl-cyclobutyl]-2-[3-[[1-[4-[1-hydroxy-1-methyl-6-(6-methyl-7-oxo-1H-pyrrolo[2,3-c]pyridin-4-yl)indan-5-yl]oxyphenyl]azetidin-3-yl]methyl]azetidin-1-yl]pyrimidine-5-carboxamide (15.1 mg, 0.01 mmol, 10% yield) was obtained as a white solid. ¹H NMR (400 MHz, CD₃OD) δ 8.72 (s, 2H), 7.86-7.79 (m, 1H), 7.72 (d, 2H, J=8.8 Hz), 7.34-7.29 (m, 1H), 7.21-7.11 (m, 2H), 7.01-6.94 (m, 1H), 6.79-6.69 (m, 3H), 6.47-6.39 (m, 2H), 6.38-6.29 (m, 1H), 4.34-4.25 (m, 3H), 4.18-4.09 (m, 1H), 3.99-3.92 (m, 2H), 3.89-3.82 (m, 2H), 3.65-3.63 (m, 3H), 3.49-3.44 (m, 2H), 3.04-2.89 (m, 1H), 2.86-2.67 (m, 3H), 2.39-1.97 (m, 4H), 1.56 (s, 3H), 1.28 (s, 6H), 1.21 (s, 6H). LC-MS: MS (ES⁺): RT=2.700 min, m/z=893.4 [M+H⁺]; LCMS Method: 05.

### Example 57—Synthesis of N-[3-(3-chloro-4-cyano-phenoxy)-2,2,4,4-tetramethyl-cyclobutyl]-2-[4-[[1-[5-[4-(1-hydroxy-1-methyl-ethyl)-2-(6-methyl-7-oxo-1H-pyrrolo[2,3-c]pyridin-4-yl)phenoxy]-2-pyridyl]azetidin-3-yl]methyl]-1-piperidyl]pyrimidine-5-carboxamide (I-333)

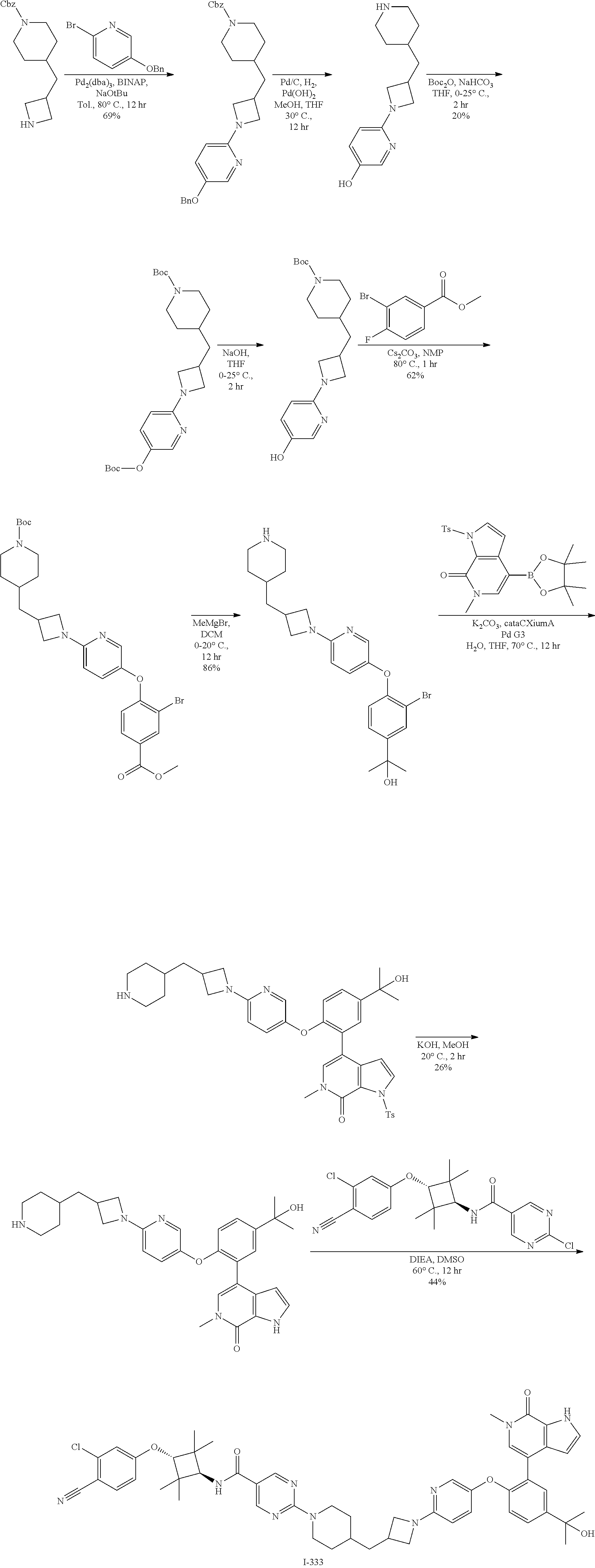

Step 1: Preparation of Benzyl 4-[[1-(5-benzyloxy-2-pyridyl) azetidin-3-yl]methyl]piperidine-1-carboxylate. A mixture of 5-benzyloxy-2-bromo-pyridine (3 g, 11.36 mmol, 1 eq), benzyl 4-(azetidin-3-ylmethyl) piperidine-1-carboxylate (3.28 g, 11.36 mmol, 1 eq), Pd₂(dba)₃ (520.06 mg, 567.93 mol, 0.05 eq), BINAP (707.26 mg, 1.14 mmol, 0.1 eq) and NaOtBu (3.60 g, 37.48 mmol, 3.3 eq) in toluene (30 mL) was degassed and purged with N₂ for 3 times, and then the mixture was stirred at 80° C. for 12 hours under N₂ atmosphere. The residue was diluted with H₂O 100 mL and extracted with EtOAc 100 mL (50 mL*2). The combined organic layers were washed with brine 100 mL, dried with Na₂SO₄, filtered and concentrated under reduced pressure to give a residue. The residue was purified by flash silica gel chromatography (ISCO®; 40 g SepaFlash® Silica Flash Column, Eluent of 0-40% Ethyl acetate/Petroleum ethergradient a 60 mL/min). Compound benzyl 4-[[1-(5-benzyloxy-2-pyridyl) azetidin-3-yl]methyl]piperidine-1-carboxylate (3.7 g, 7.85 mmol, 69.07% yield) was obtained as a brown oil.

Step 2: Preparation of 6-[3-(4-piperidylmethyl)azetidin-1-yl]pyridin-3-ol. To a solution of benzyl 4-[[1-(5-benzyloxy-2-pyridyl)azetidin-3-yl]methyl]piperidine-1-carboxylate (3.5 g, 7.42 mmol, 1 eq) in MeOH (15 mL) and THF (15 mL) was added Pd/C (700.00 mg, 657.77 mol, 10% purity, 8.86e-2 eq) and Pd(OH)₂ (900 mg, 6.41 mmol, 8.63e-1 eq) under N₂ atmosphere. The suspension was degassed and purged with H₂ for 3 times. The mixture was stirred under H₂ (50 Psi) at 30° C. for 12 hours. The organic phase was separated, washed with THF 50 mL then concentrated under reduced pressure to remove solvent. Compound 6-[3-(4-piperidylmethyl)azetidin-1-yl]pyridin-3-ol (2.5 g, crude) was obtained as a brown solid.

Step 3: Preparation of tert-butyl 4-[[1-(5-tert-butoxycarbonyloxy-2-pyridyl)azetidin-3-yl]methyl]piperidine-1-carboxylate. A mixture of 6-[3-(4-piperidylmethyl)azetidin-1-yl]pyridin-3-ol (2.5 g, 10.11 mmol, 1 eq) and tert-butoxycarbonyl tert-butyl carbonate (2.21 g, 10.11 mmol, 2.32 mL, 1 eq) in THF (20 mL) was added NaHCO₃ (1.70 g, 20.22 mmol, 786.62 μL, 2 eq) dropwise slowly at 0° C., and then the mixture was stirred at 25° C. for 2 hours. The residue was diluted with H₂O 80 mL and extracted with EtOAc 100 mL (50 mL*2). The combined organic layers were washed with brine 80 mL, dried over Na₂SO₄, filtered and concentrated under reduced pressure to give a residue. The residue was purified by flash silica gel chromatography (ISCO®; 20 g SepaFlash® Silica Flash Column, Eluent of 0˜10% Dichloromethane/Methanol gradient®50 mL/min). Compound tert-butyl 4-[[1-(5-tert-butoxycarbonyloxy-2-pyridyl)azetidin-3-yl]methyl]piperidine-1-carboxylate (800 mg, 1.79 mmol, 17.68% yield) was obtained as a brown oil.

Step 4: Preparation of tert-butyl 4-[[1-(5-hydroxy-2-pyridyl) azetidin-3-yl]methyl]piperidine-1-carboxylate. A mixture of tert-butyl 4-[[1-(5-tert-butoxycarbonyloxy-2-pyridyl)azetidin-3-yl]methyl]piperidine-1-carboxylate (0.8 g, 1.79 mmol, 1 eq) in THF (30 mL) was added NaOH (142.99 mg, 3.57 mmol, 2 eq) dropwise slowly at 0° C., and then the mixture was stirred at 25° C. for 2 hours. The residue was diluted with H₂O 30 mL and added AcOH to pH=8, extracted with DCM 90 mL (30 mL*3). The combined organic layers were washed with brine 50 mL, dried over anhydrous Na₂SO₄, filtered and concentrated under reduced pressure to give a residue. Compound tert-butyl 4-[[1-(5-hydroxy-2-pyridyl) azetidin-3-yl]methyl]piperidine-1-carboxylate (800 mg, crude) was obtained as a brown oil and used into the next step without further purification.

Step 5: Preparation of tert-butyl 4-[[1-[5-(2-bromo-4-methoxycarbonyl-phenoxy)-2-pyridyl]azetidin-3-yl]methyl]piperidine-1-carboxylate. To a solution of tert-butyl 4-[[1-(5-hydroxy-2-pyridyl)azetidin-3-yl]methyl]piperidine-1-carboxylate (800 mg, 2.30 mmol, 1 eq) in NMP (10 mL) were added Cs₂CO₃ (1.50 g, 4.60 mmol, 2 eq) and methyl 3-bromo-4-fluoro-benzoate (590.21 mg, 2.53 mmol, 1.1 eq). The mixture was stirred at 80° C. for 1 hour. The residue was diluted with H₂O 50 mL and extracted with EtOAc 60 mL (30 mL*2). The combined organic layers were washed with brine 50 mL, dried over Na₂SO₄, filtered and concentrated under reduced pressure to give a residue. The residue was purified by flash silica gel chromatography (ISCO®; 12 g SepaFlash® Silica Flash Column, Eluent of 0˜20% Ethyl acetate/Petroleum ether gradient®50 mL/min). Compound tert-butyl 4-[[1-[5-(2-bromo-4-methoxycarbonyl-phenoxy)-2-pyridyl]azetidin-3-yl]methyl]piperidine-1-carboxylate (660 mg, 1.18 mmol, 51.14% yield) was obtained as a light yellow oil.

Step 6: Preparation of 2-[3-bromo-4-[[6-[3-(4-piperidylmethyl)azetidin-1-yl]-3-pyridyl]oxy]phenyl]propan-2-ol. To a solution of tert-butyl 4-[[1-[5-(2-bromo-4-methoxycarbonyl-phenoxy)-2-pyridyl]azetidin-3-yl]methyl]piperidine-1-carboxylate (1.2 g, 2.14 mmol, 1 eq) in DCM (50 mL) was added MeMgBr (3 M, 14.27 mL, 20 eq) at 0° C. Then the mixture was warmed to 20° C. and stirred for 12 hours. The residue was quenched by addition NH₄Cl (aq.) 40 mL at 0° C. and diluted H₂O 50 mL. The combined organic layers were washed with DCM 150 mL (50 mL*3), dried over Na₂SO₄, filtered and concentrated under reduced pressure to give a residue. Compound 2-[3-bromo-4-[[6-[3-(4-piperidylmethyl)azetidin-1-yl]-3-pyridyl]oxy]phenyl]propan-2-ol (0.85 g, 1.85 mmol, 76.08% yield) was obtained as a light yellow gum, and used into the next step without further purification.

Step 7: Preparation of 4-[5-(1-hydroxy-1-methyl-ethyl)-2-[[6-[3-(4-piperidylmethyl)azetidin-1-yl]-3-pyridyl]oxy]phenyl]-6-methyl-1-(p-tolylsulfonyl)pyrrolo[2,3-c]pyridin-7-one. A mixture of 2-[3-bromo-4-[[6-[3-(4-piperidylmethyl)azetidin-1-yl]-3-pyridyl]oxy]phenyl]propan-2-ol (0.85 g, 1.85 mmol, 1 eq), 6-methyl-1-(p-tolylsulfonyl)-4-(4,4,5,5-tetramethyl-1,3,2-dioxaborolan-2-yl)pyrrolo[2,3-c]pyridin-7-one (0.85 g, 1.98 mmol, 1.07 eq), K₂CO₃ (765.46 mg, 5.54 mmol, 3 eq), [2-(2-aminophenyl)phenyl]palladium(1+);bis(1-adamantyl)-butyl-phosphane;methanesulfonate (67.23 mg, 92.31 mol, 0.05 eq) in THF (8 mL) and H₂O (1 mL) was degassed and purged with N₂ for 3 times, and then the mixture was stirred at 70° C. for 12 hours under N₂ atmosphere. The residue was diluted with H₂O 60 mL and extracted with EtOAc 90 mL (30 mL*3). The combined organic layers were washed with brine 50 mL, dried over Na₂SO₄, filtered and concentrated under reduced pressure to give a residue. Compound 4-[5-(1-hydroxy-1-methyl-ethyl)-2-[[6-[3-(4-piperidylmethyl)azetidin-1-yl]-3-pyridyl]oxy]phenyl]-6-methyl-1-(p-tolylsulfonyl)pyrrolo[2,3-c]pyridin-7-one (1.5 g, crude) was obtained as a yellow gum and used into the next step without further purification.

Step 8: Preparation of 4-[5-(1-hydroxy-1-methyl-ethyl)-2-[[6-[3-(4-piperidylmethyl)azetidin-1-yl]-3-pyridyl]oxy]phenyl]-6-methyl -1H-pyrrolo[2,3-c]pyridin-7-one. A mixture of 4-[5-(1-hydroxy-1-methyl-ethyl)-2-[[6-[3-(4-piperidylmethyl)azetidin-1-yl]-3-pyridyl]oxy]phenyl]-6-methyl-1-(p-tolylsulfonyl) pyrrolo[2,3-c]pyridin-7-one (1.5 g, 2.20 mmol, 1 eq) and KOH (617.14 mg, 11.00 mmol, 5 eq) in MeOH (20 mL), the mixture was stirred at 20° C. for 2 hours. The residue was diluted with H₂O 50 mL and extracted with EtOAc 80 mL (40 mL*2). The combined organic layers were washed with brine 50 mL, dried over Na₂SO₄, filtered and concentrated under reduced pressure to give a residue. The residue was purified by prep-HPLC (FA condition; column: Phenomenex luna C18 150*40 mm* 15 um;mobile phase: [water(FA)-ACN];gradient:2%-32% B over 15 min). Compound 4-[5-(1-hydroxy-1-methyl-ethyl)-2-[[6-[3-(4-piperidylmethyl)azetidin-1-yl]-3-pyridyl]oxy]phenyl]-6-methyl -1H-pyrrolo[2,3-c]pyridin-7-one (300 mg, 568.55 mol, 25.84% yield) was obtained as a white solid.

Step 9: Preparation of N-[3-(3-chloro-4-cyano-phenoxy)-2,2,4,4-tetramethyl-cyclobutyl]-2-[4-[[1-[5-[4-(1-hydroxy-1-methyl-ethyl)-2-(6-methyl-7-oxo-1H-pyrrolo[2,3-c]pyridin-4-yl)phenoxy]-2-pyridyl]azetidin-3-yl]methyl]-1-piperidyl]pyrimidine-5-carboxamide. To a solution of 4-[5-(1-hydroxy-1-methyl-ethyl)-2-[[6-[3-(4-piperidylmethyl)azetidin-1-yl]-3-pyridyl]oxy]phenyl]-6-methyl-¹H-pyrrolo[2,3-c]pyridin-7-one (50 mg, 94.76 mol, 1 eq), 2-chloro-N-[3-(3-chloro-4-cyano-phenoxy)-2,2,4,4-tetramethyl-cyclobutyl]pyrimidine-5-carboxamide (40 mg, 95.40 μmol, 1.01 eq) in DMSO (1 mL) was added DIEA (24.49 mg, 189.52 mol, 33.01 μL, 2 eq). The mixture was stirred at 60° C. for 12 hours. The reaction mixture was filtered. The residue was purified by prep-HPLC (FA condition; column: Phenomenex luna C18 150*25 mm* 10 um;mobile phase: [water(FA)-ACN];gradient:42%-72% B over 10 min). Compound N-[3-(3-chloro-4-cyano-phenoxy)-2,2,4,4-tetramethyl-cyclobutyl]-2-[4-[[1-[5-[4-(1-hydroxy-1-methyl-ethyl)-2-(6-methyl-7-oxo-1H-pyrrolo[2,3-c]pyridin-4-yl)phenoxy]-2-pyridyl]azetidin-3-yl]methyl]-1-piperidyl]pyrimidine-5-carboxanide (38.36 mg, 41.59 mol, 43.89% yield, 98.71% purity) was obtained as a white solid. ¹H NMR (400 MHz, CDCl₃) δ (ppm) 9.70-9.55 (m, 1H), 8.70 (s, 2H), 7.87 (br d, J=1.6 Hz, 1H), 7.62-7.55 (m, 2H), 7.44-7.36 (m, 1H), 7.23 (t, J=2.8 Hz, 1H), 7.14-7.07 (m, 2H), 6.97 (d, J=2.4 Hz, 1H), 6.87-6.79 (m, 2H), 6.39 (t, J=2.4 Hz, 1H), 6.21 (d, J=8.8 Hz, 1H), 5.92 (d, J=8.0 Hz, 1H), 4.86 (br d, J=13.2 Hz, 2H), 4.15-4.03 (m, 4H), 3.67 (s, 3H), 3.64-3.58 (m, 2H), 2.98-2.80 (m, 3H), 1.78 (br d, J=12.0 Hz, 3H), 1.66 (br d, J=7.2 Hz, 4H), 1.63 (s, 6H), 1.25 (s, 6H), 1.22 (s, 6H). LC-MS: MS (ES⁺): RT=2.129 min, m/z=912.4[M+H⁺]; LCMS Method: 25.

### Example 58—Synthesis of N-[3-(3-chloro-4-cyano-phenoxy)-2,2,4,4-tetramethyl-cyclobutyl]-2-[4-[[1-[1-[4-(1-hydroxy-1-methyl-ethyl)-2-(6-methyl-7-oxo-1H-pyrrolo [2, 3-c]pyridin-4-yl) phenyl]-6-oxo-3-pyridyl]azetidin-3-yl]methyl]-1-piperidyl]pyrimidine-5-carboxamide (I-344)

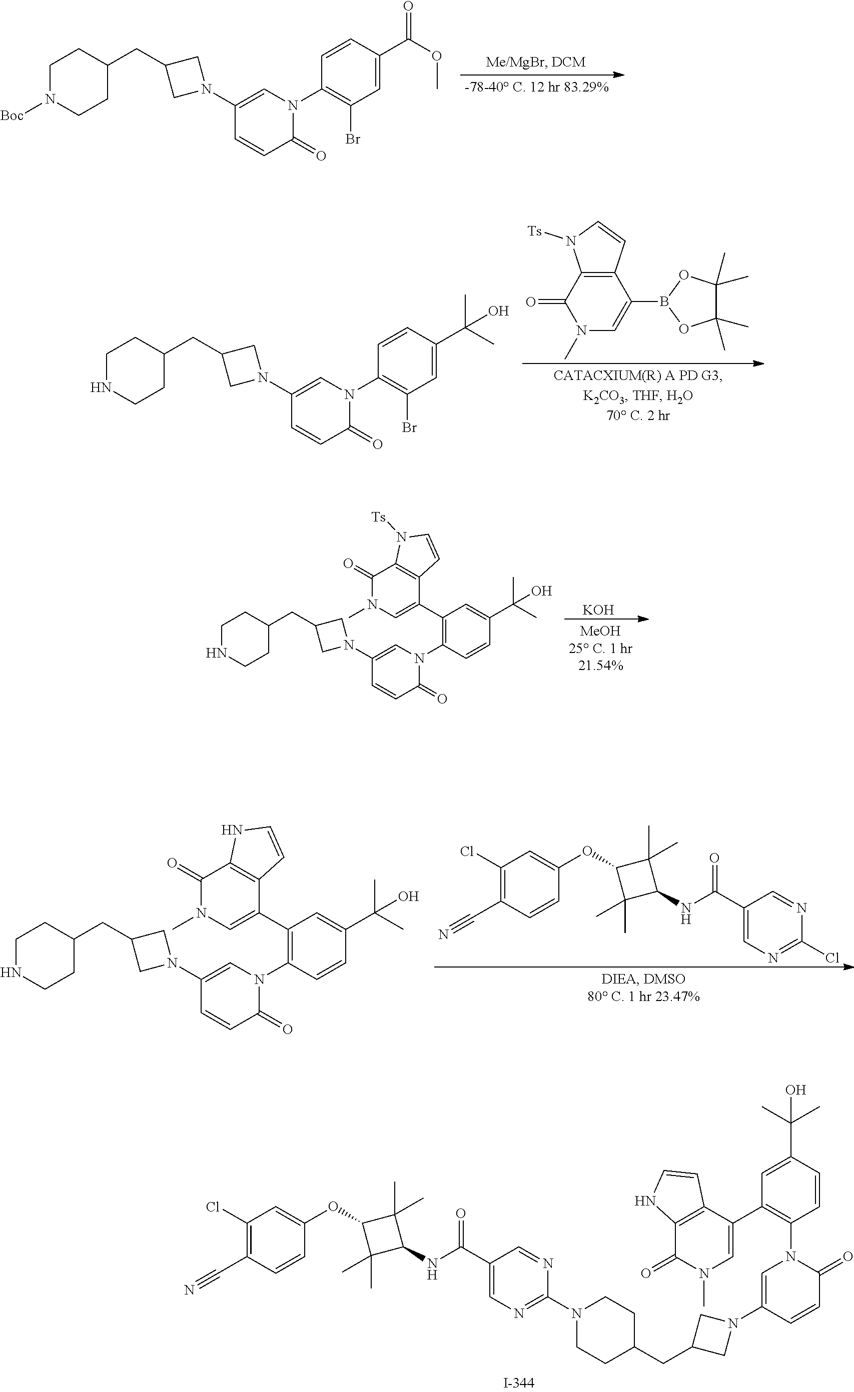

Step 1: Preparation of 1-[2-bromo-4-(1-hydroxy-1-methyl-ethyl)phenyl]-5-[3-(4-piperidylmethyl) azetidin-1-yl]pyridin-2-one. To a solution of tert-butyl 4-[[1-[1-(2-bromo-4-methoxycarbonyl-phenyl)-6-oxo-3-pyridyl]azetidin-3-yl]methyl]piperidine-1-carboxylate (190 mg, 339.00 μmol, 1 eq) in DCM (5 mL) was added MeMgBr (3 M, 2.2 mL, 19.47 eq) at −78° C. under N₂. The mixture was stirred 40° C. for 12 hours. The reaction mixture was quenched by addition NH₄Cl. aq 5 mL, and then extracted with DCM 10 mL. The organic layer was washed with brine 10 mL, dried over Na₂SO₄, filtered and concentrated under reduced pressure to give a residue. The crude product 1-[2-bromo-4-(1-hydroxy-1-methyl-ethyl)phenyl]-5-[3-(4-piperidylmethyl) azetidin-1-yl]pyridin-2-one (130 mg, 282.36 μmol, 83.29% yield) was obtained as a brown solid and used into the next step without further purification.

Step 2: Preparation of 4-[5-(1-hydroxy-1-methyl-ethyl)-2-[2-oxo-5-[3-(4-piperidylmethyl) azetidin-1-yl]-1-pyridyl]phenyl]-6-methyl-1-(p-tolylsulfonyl) pyrrolo [2,3-c]pyridin-7-one. A mixture of 1-[2-bromo-4-(1-hydroxy-1-methyl-ethyl)phenyl]-5-[3-(4-piperidylmethyl)azetidin-1-yl]pyridin-2-one (130 mg, 282.36 mol, 1 eq), 6-methyl-1-(p-tolylsulfonyl)-4-(4,4,5,5-tetramethyl-1,3,2-dioxaborolan-2-yl) pyrrolo[2,3-c]pyridin-7-one (130.00 mg, 303.52 mol, 1.07 eq), CATACXIUM(R) A PD G3 (20 mg, 27.46 μmol, 9.73e-2 eq), K₂CO₃ (117.07 mg, 847.08 gmol, 3 eq) in THF (2 mL) and H₂O(0.2 mL) was degassed and purged with N₂ for 3 times, and then the mixture was stirred at 70° C. for 2 hours under N₂ atmosphere. The reaction mixture was diluted with H₂O 3 mL and extracted with EA 8 mL (4 mL*2). The combined organic layers were washed with brine 10 mL (5 mL*2), dried over Na₂SO₄, filtered and concentrated under reduced pressure to give a residue. The crude product 4-[5-(1-hydroxy-1-methyl-ethyl)-2-[2-oxo-5-[3-(4-piperidylmethyl) azetidin-1-yl]-1-pyridyl]phenyl]-6-methyl-1-(p-tolylsulfonyl) pyrrolo [2,3-c]pyridin-7-one (180 mg, crude) was obtained as a brown solid and used into the next step without further purification.

Step 3: Preparation of 4-[5-(1-hydroxy-1-methyl-ethyl)-2-[2-oxo-5-[3-(4-piperidylmethyl) azetidin-1-yl]-1-pyridyl]phenyl]-6-methyl-11H-pyrrolo [2,3-c]pyridin-7-one. To a solution of 4-[5-(1-hydroxy-1-methyl-ethyl)-2-[2-oxo-5-[3-(4-piperidylmethyl)azetidin-1-yl]-1-pyridyl]phenyl]-6-methyl-1-(p-tolylsulfonyl) pyrrolo[2,3-c]pyridin-7-one (180 mg, 263.99 μmol, 1 eq) in MeOH (5 mL) was added KOH (300 mg, 5.35 mmol, 20.25 eq). The mixture was stirred at 20° C. for 1 hour. The reaction mixture was diluted with H₂O 3 mL and extracted with DCM 15 mL (5 mL*3). The combined organic layers were dried over Na₂SO₄, filtered and concentrated under reduced pressure to give a residue. The residue was purified by prep-HPLC (FA condition:column: Phenomenex luna C18 150*25 mm* 10 um;mobile phase: [water(FA)-ACN]; gradient:5%-35% B over 10 min). Compound 4-[5-(1-hydroxy-1-methyl-ethyl)-2-[2-oxo-5-[3-(4-piperidylmethyl) azetidin-1-yl]-1-pyridyl]phenyl]-6-methyl-1H-pyrrolo [2,3-c]pyridin-7-one (30 mg, 56.86 μmol, 21.54% yield) was obtained as a green solid.

Step 4: Preparation of N-[3-(3-chloro-4-cyano-phenoxy)-2,2,4,4-tetramethyl-cyclobutyl]-2-[4-[[1-[1-[4-(1-hydroxy-1-methyl-ethyl)-2-(6-methyl-7-oxo-1H-pyrrolo [2, 3-c]pyridin-4-yl) phenyl]-6-oxo-3-pyridyl]azetidin-3-yl]methyl]-1-piperidyl]pyrimidine-5-carboxamide. To a solution of 4-[5-(1-hydroxy-1-methyl-ethyl)-2-[2-oxo-5-[3-(4-piperidylmethyl)azetidin-1-yl]-1-pyridyl]phenyl]-6-methyl-1H-pyrrolo [2,3-c]pyridin-7-one (30 mg, 56.86 μmol, 1 eq) in DMSO (0.5 mL) was added DIEA (7.35 mg, 56.86 μmol, 9.90 μL, 1 eq) and 2-chloro-N-[3-(3-chloro-4-cyano-phenoxy)-2,2,4,4-tetramethyl-cyclo butyl]pyrimidine-5-carboxamide (25 mg, 59.62 mol, 1.05 eq). The mixture was stirred at 80° C. for 1 hour. The reaction mixture was concentrated under reduced pressure to remove DIEA. The residue was purified by prep-HPLC (neutral condition: column: Waters xbridge 150*25 mm 10 um;mobile phase: [water(NH₄HCO₃)-ACN];gradient:44%-74% B over 10 min). Compound N-[3-(3-chloro-4-cyano-phenoxy)-2,2,4,4-tetramethyl-cyclobutyl]-2-[4-[[1-[1-[4-(1-hydroxy-1-methyl-ethyl)-2-(6-methyl-7-oxo-1H-pyrrolo [2, 3-c]pyridin-4-yl) phenyl]-6-oxo-3-pyridyl]azetidin-3-yl]methyl]-1-piperidyl]pyrimidine-5-carboxamide (13.4 mg, 13.34 gmol, 23.47% yield, 90.65% purity) was obtained as a green solid. ¹H NMR (400 MHz, CD₃OD) δ (ppm)=8.73 (s, 2H), 7.76-7.67 (m, 3H), 7.46 (d, J=8.0 Hz, 1H), 7.27 (d, J=2.8 Hz, 1H), 7.13 (d, J=2.4 Hz, 1H), 7.10-7.03 (m, 1H), 7.01-6.96 (m, 2H), 6.48 (d, J=9.2 Hz, 1H), 6.40-6.34 (m, 1H), 6.25 (d, J=2.8 Hz, 1H), 4.60 (s, 2H), 4.27 (s, 1H), 4.13 (s, 1H), 3.68 (s, 2H), 3.57 (s, 3H), 3.03 (br t, J=6.4 Hz, 1H), 2.98-2.90 (m, 2H), 2.80-2.66 (m, 2H), 1.78-1.68 (m, 2H), 1.61 (s, 7H), 1.50-1.42 (m, 2H), 1.28 (s, 6H), 1.22-1.16 (m, 8H). LC-MS: MS (ES⁺): RT=2.283 min, m/z=910.4 [M+H]; LCMS Method: 01.

### Example 59—Synthesis of N-[3-(3-chloro-4-cyano-phenoxy)-2,2,4,4-tetramethyl-cyclobutyl]-2-[4-[[1-[6-[4-(1-hydroxy-1-methyl-ethyl)-2-(6-methyl-7-oxo-1H-pyrrolo [2,3-c]pyridin-4-yl) phenoxy]-3-pyridyl]azetidin-3-yl]methyl]-1-piperidyl]pyrimidine-5-carboxamide (I-345)

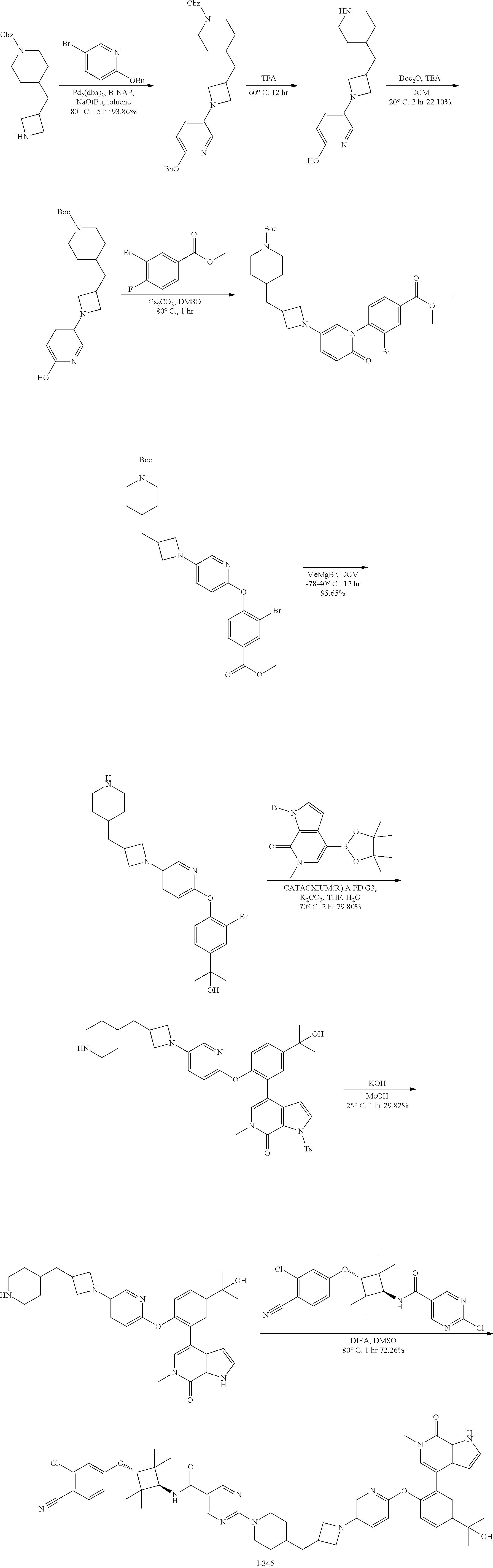

Step 1: Preparation of Benzyl 4-[[1-(6-benzyloxy-3-pyridyl) azetidin-3-yl]methyl]piperidine-1-carboxylate. A mixture of benzyl 4-(azetidin-3-ylmethyl)piperidine-1-carboxylate (10 g, 24.85 mmol, 1 eq, TFA), 2-benzyloxy-5-bromo-pyridine (6.56 g, 24.85 mmol, 1 eq), Pd₂(dba)₃ (1.00 g, 1.09 mmol, 4.39e-2 eq), BINAP (1.60 g, 2.57 mmol, 1.03e-1 eq) and NaOtBu (15.00 g, 156.08 mmol, 6.28 eq) in toluene (200 mL) was degassed and purged with N₂ for 3 times, and then the mixture was stirred at 80° C. for 15 hours under N₂ atmosphere. The reaction mixture was diluted with H₂O 100 mL and extracted with EA 200 mL (100 mL*2). The combined organic layers were washed with brine 200 mL, dried over Na₂SO₄, filtered and concentrated under reduced pressure to give a residue. The residue was purified by flash silica gel chromatography (ISCO®; 80 g SepaFlash® Silica Flash Column, Eluent of 0˜33% Ethyl acetate/Petroleum ethergradient a 100 m/min). Compound benzyl 4-[[1-(6-benzyloxy-3-pyridyl) azetidin-3-yl]methyl]piperidine-1-carboxylate (11 g, 23.33 mmol, 93.86% yield) was obtained as a brown oil.

Step 2: Preparation of 5-[3-(4-piperidylmethyl) azetidin-1-yl]pyridin-2-ol. A solution of benzyl 4-[[1-(6-benzyloxy-3-pyridyl) azetidin-3-yl]methyl]piperidine-1-carboxylate (11 g, 23.33 mmol, 1 eq) in TFA (0.5 mL) was stirred at 60° C. for 12 hours. The reaction mixture was concentrated under reduced pressure to give a residue. The crude product 5-[3-(4-piperidylmethyl) azetidin-1-yl]pyridin-2-ol (8 g, crude, TFA) was obtained as a brown oil and used into the next step without further purification.

Step 3: Preparation of tert-butyl 4-[[1-(6-hydroxy-3-pyridyl) azetidin-3-yl]methyl]piperidine-1-carboxylate. To a solution of 5-[3-(4-piperidylmethyl)azetidin-1-yl]pyridin-2-ol (8 g, 22.14 mmol, 1 eq, TFA) in DCM (200 mL) was added ditert-butyl carbonate (5.79 g, 33.21 mmol, 1.5 eq) and TEA (13.27 g, 131.13 mmol, 18.25 mL, 5.92 eq). The mixture was stirred at 20° C. for 2 hours. The reaction mixture was concentrated under reduced pressure to give a residue. The residue was purified by flash silica gel chromatography (ISCO®; 120 g SepaFlash Silica Flash Column, Eluent of 0˜10% Methanol/Dichloromethane:®100 mL/min). And the product was further purified by prep-HPLC (neutral condition: column: Waters Xbridge C18 150*50 mm* 10 um; mobile phase: [water(NH₄HCO₃)-ACN];gradient:21%-51% B over min). Compound tert-butyl 4-[[1-(6-hydroxy-3-pyridyl) azetidin-3-yl]methyl]piperidine-1-carboxylate (1.7 g, 4.89 mmol, 22.10% yield) was obtained as a green solid.

Step 4: Preparation of tert-butyl 4-[[1-[6-(2-bromo-4-methoxycarbonyl-phenoxy)-3-pyridyl]azetidin-3-yl]methyl]piperidine-1-carboxylate. To a solution of tert-butyl 4-[[1-(6-hydroxy-3-pyridyl) azetidin-3-yl]methyl]piperidine-1-carboxylate (1.3 g, 3.74 mmol, 1 eq) and methyl 3-bromo-4-fluoro-benzoate (1 g, 4.29 mmol, 1.15 eq) in DMS0 (20 mL) was added Cs₂CO₃ (2.5 g, 7.67 mmol, 2.05 eq). The mixture was stirred at 80° C. for 1 hour. The reaction mixture was diluted with H₂O 20 mL and extracted with EA 60 mL (20 mL*3). The combined organic layers were washed with aqueous NaCl 60 mL (20 mL*3), dried over Na₂SO₄, filtered and concentrated under reduced pressure to give a residue. The residue was purified by prep-HPLC (neutral condition:column: Waters Xbridge BEH C18 150*25 mm*5 um;mobile phase: [water(NH₄HCO₃)-ACN];gradient: 64%-94% B over 10 min). Compound tert-butyl 4-[[1-[6-(2-bromo-4-methoxycarbonyl-phenoxy)-3-pyridyl]azetidin-3-yl]methyl]piperidine-1-carboxylate (1.4 g, 2.50 mmol, 66.76% yield) was obtained as a green solid. Tert-butyl 4-[[1-[1-(2-bromo-4-methoxycarbonyl-phenyl)-6-oxo-3-pyridyl]azetidin-3-yl]methyl]piperidine-1-carboxylate (190 mg, 339.00 mol) was delivered as a side product.

Step 5: Preparation of 2-[3-bromo-4-[[5-[3-(4-piperidylmethyl) azetidin-1-yl]-2-pyridyl]oxy]phenyl]propan-2-ol. To a solution of tert-butyl 4-[[1-[6-(2-bromo-4-methoxycarbonyl-phenoxy)-3-pyridyl]azetidin-3-yl]methyl]piperidine-1-carboxylate (1.4 g, 2.50 mmol, 1 eq) in DCM (30 mL) was added MeMgBr (3 M, 16.80 mL, 20.18 eq) at −78° C. under N₂. The mixture was stirred 40° C. for 12 hours. The reaction mixture was quenched by addition NH₄C₁. aq 20 mL, and then extracted with DCM 30*3 mL. The organic layer was washed with brine 30 mL, dried over Na₂SO₄, filtered and concentrated under reduced pressure to give a residue. The crude product 2-[3-bromo-4-[[5-[3-(4-piperidylmethyl) azetidin-1-yl]-2-pyridyl]oxy]phenyl]propan-2-ol (1.1 g, 2.39 mmol, 95.65% yield) was obtained as a yellow gum and used into the next step without further purification.

Step 6: Preparation of 4-[5-(1-hydroxy-1-methyl-ethyl)-2-[[5-[3-(4-piperidylmethyl) azetidin-1-yl]-2-pyridyl]oxy]phenyl]-6-methyl-1-(p-tolylsulfonyl) pyrrolo[2,3-c]pyridin-7-one. A mixture of 2-[3-bromo-4-[[5-[3-(4-piperidylmethyl) azetidin-1-yl]-2-pyridyl]oxy]phenyl]propan-2-ol (1.1 g, 2.39 mmol, 1 eq), 6-methyl-1-(p-tolylsulfonyl)-4-(4,4,5,5-tetramethyl-1,3,2-dioxaborolan-2-yl)pyrrolo[2,3-c]pyridin-7-one (1.10 g, 2.57 mmol, 1.07 eq), CATACXIUM(R) A PD G3 (93.08 mg, 127.81 μmol, 5.35e-2 eq), K₂CO₃ (990.63 mg, 7.17 mmol, 3 eq) in THF (20 mL) and H₂O (2 mL) was degassed and purged with N₂ for 3 times, and then the mixture was stirred at 70° C. for 2 hours under N₂ atmosphere. The reaction mixture was diluted with H₂O 20 mL and extracted with EA 60 mL (20 mL*3). The combined organic layers were washed with brine 50 mL, dried over Na₂SO₄, filtered and concentrated under reduced pressure to give a residue. The crude product 4-[5-(1-hydroxy-1-methyl-ethyl)-2-[[5-[3-(4-piperidylmethyl) azetidin-1-yl]-2-pyridyl]oxy]phenyl]-6-methyl-1-(p-tolylsulfonyl) pyrrolo[2,3-c]pyridin-7-one (1.3 g, 1.91 mmol, 79.80% yield) was obtained as a brown solid and used into the next step without further purification.

Step 7: Preparation of 4-[5-(1-hydroxy-1-methyl-ethyl)-2-[[5-[3-(4-piperidylmethyl) azetidin-1-yl]-2-pyridyl]oxy]phenyl]-6-methyl-1H-pyrrolo [2,3-c]pyridin-7-one. To a solution of 4-[5-(1-hydroxy-1-methyl-ethyl)-2-[[5-[3-(4-piperidylmethyl)azetidin-1-yl]-2-pyridyl]oxy]phenyl]-6-methyl-1-(p-tolylsulfonyl) pyrrolo[2,3-c]pyridin-7-one (1.3 g, 1.91 mmol, 1 eq) in MeOH (20 mL) was added KOH (2.5 g, 44.56 mmol, 23.37 eq). The mixture was stirred at 25° C. for 1 hour. The reaction mixture was diluted with H₂O 15 mL and extracted with DCM 45 mL (15 mL*3). The combined organic layers were dried over Na₂SO₄, filtered and concentrated under reduced pressure to give a residue. The residue was purified by prep-HPLC (neutral condition:column: Waters Xbridge C18 150*50 mm* 10 um;mobile phase: [water(NH₄HCO₃)-ACN];gradient:12%-42% B over 10 min). Compound 4-[5-(1-hydroxy-1-methyl-ethyl)-2-[[5-[3-(4-piperidylmethyl) azetidin-1-yl]-2-pyridyl]oxy]phenyl]-6-methyl-1H-pyrrolo [2,3-c]pyridin-7-one (300 mg, 568.55 μmol, 29.82% yield) was obtained as a white solid.

Step 8: Preparation of N-[3-(3-chloro-4-cyano-phenoxy)-2,2,4,4-tetramethyl-cyclobutyl]-2-[4-[[1-[6-[4-(1-hydroxy-1-methyl-ethyl)-2-(6-methyl-7-oxo-1H-pyrrolo [2,3-c]pyridin-4-yl) phenoxy]-3-pyridyl]azetidin-3-yl]methyl]-1-piperidyl]pyrimidine-5-carboxamide. To a solution of 4-[5-(1-hydroxy-1-methyl-ethyl)-2-[[5-[3-(4-piperidyl methyl)azetidin-1-yl]-2-pyridyl]oxy]phenyl]-6-methyl-1H-pyrrolo [2,3-c]pyridin-7-one (40 mg, 75.81 gmol, 1 eq) in DMSO (0.5 mL) was added DIEA (29.39 mg, 227.42 μmol, 39.61 μL, 3 eq) and 2-chloro-N-[3-(3-chloro-4-cyano-phenoxy)-2,2,4,4-tetramethyl-cyclobutyl]pyrimidine-5-carboxamide (32.00 mg, 76.32 ltmol, 1.01 eq). The mixture was stirred at 80° C. for 1 hour. The reaction mixture was concentrated under reduced pressure to remove DIEA. The residue was purified by prep-HPLC (FA condition: column: Phenomenex luna C18 150*25 mm* 10 um;mobile phase: [water(FA)-ACN];gradient:61%-91% B over 10 min). Compound N-[3-(3-chloro-4-cyano-phenoxy)-2,2,4,4-tetramethyl-cyclobutyl]-2-[4-[[1-[6-[4-(1-hydroxy-1-methyl-ethyl)-2-(6-methyl-7-oxo-1H-pyrrolo [2,3-c]pyridin-4-yl) phenoxy]-3-pyridyl]azetidin-3-yl]methyl]-1-piperidyl]pyrimidine-5-carboxamide (51.64 mg, 54.78 mol, 72.26% yield, 96.58% purity) was obtained as a yellow solid. ¹H NMR (400 MHz, CD₃OD) δ (ppm)=8.72 (s, 2H), 7.72 (d, J=8.8 Hz, 1H), 7.64 (d, J=2.4 Hz, 1H), 7.54-7.47 (m, 1H), 7.29 (d, J=2.8 Hz, 1H), 7.19 (d, J=2.8 Hz, 1H), 7.15-7.11 (m, 2H), 7.06 (d, J=8.6 Hz, 1H), 7.01-6.95 (m, 1H), 6.85-6.79 (m, 1H), 6.54 (d, J=8.8 Hz, 1H), 6.33 (d, J=2.8 Hz, 1H), 4.85-4.82 (m, 1H), 4.60 (s, 1H), 4.27 (s, 1H), 4.13 (s, 1H), 3.95 (t, J=7.2 Hz, 2H), 3.60 (s, 3H), 3.38 (t, J=6.4 Hz, 2H), 3.00-2.84 (m, 3H), 1.78 (br d, J=12.4 Hz, 2H), 1.70-1.55 (m, 9H), 1.28 (s, 6H), 1.23-1.11 (m, 8H). LC-MS: MS (ES⁺): RT=2.681 min, m/z=910.4 [M+H]; LCMS Method: 25.

### Example 60—Synthesis of N-[3-(3-chloro-4-cyano-phenoxy)-2,2,4,4-tetramethyl-cyclobutyl]-2-[4-[[4-[2-(6-methyl-7-oxo-1H-pyrrolo[2,3-c]pyridin-4-yl)phenoxy]phenoxy]methyl]-1-piperidyl]pyrimidine-5-carboxamide (I-356)

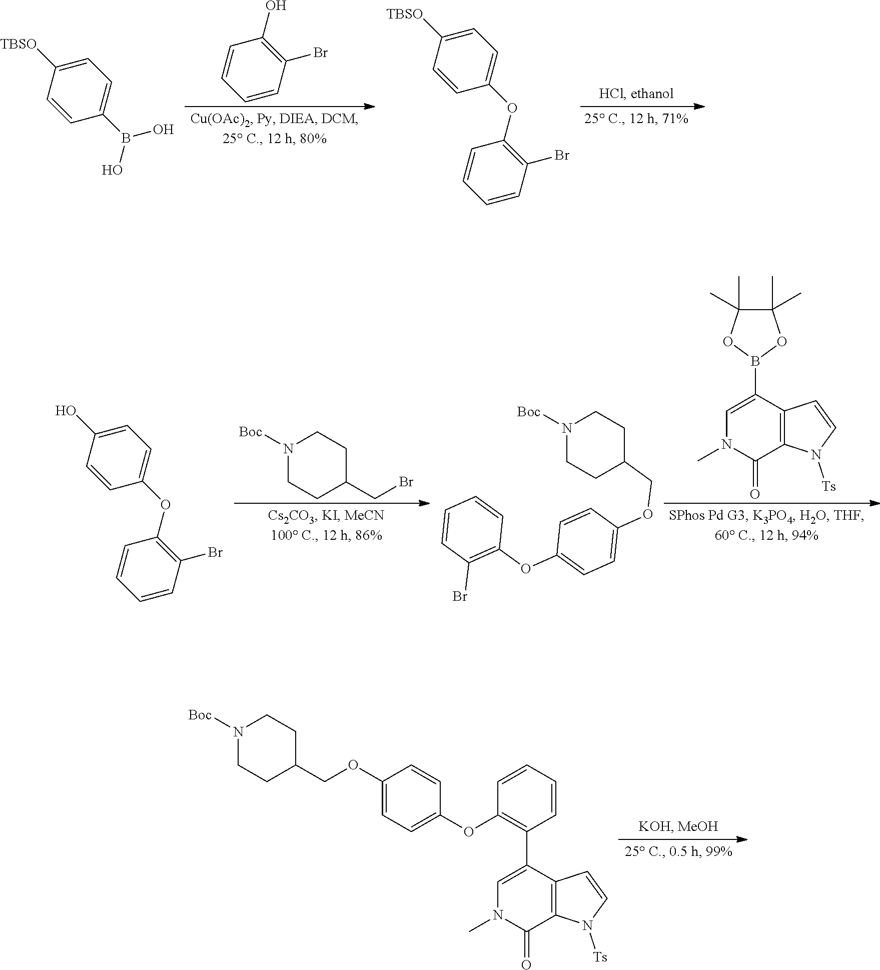

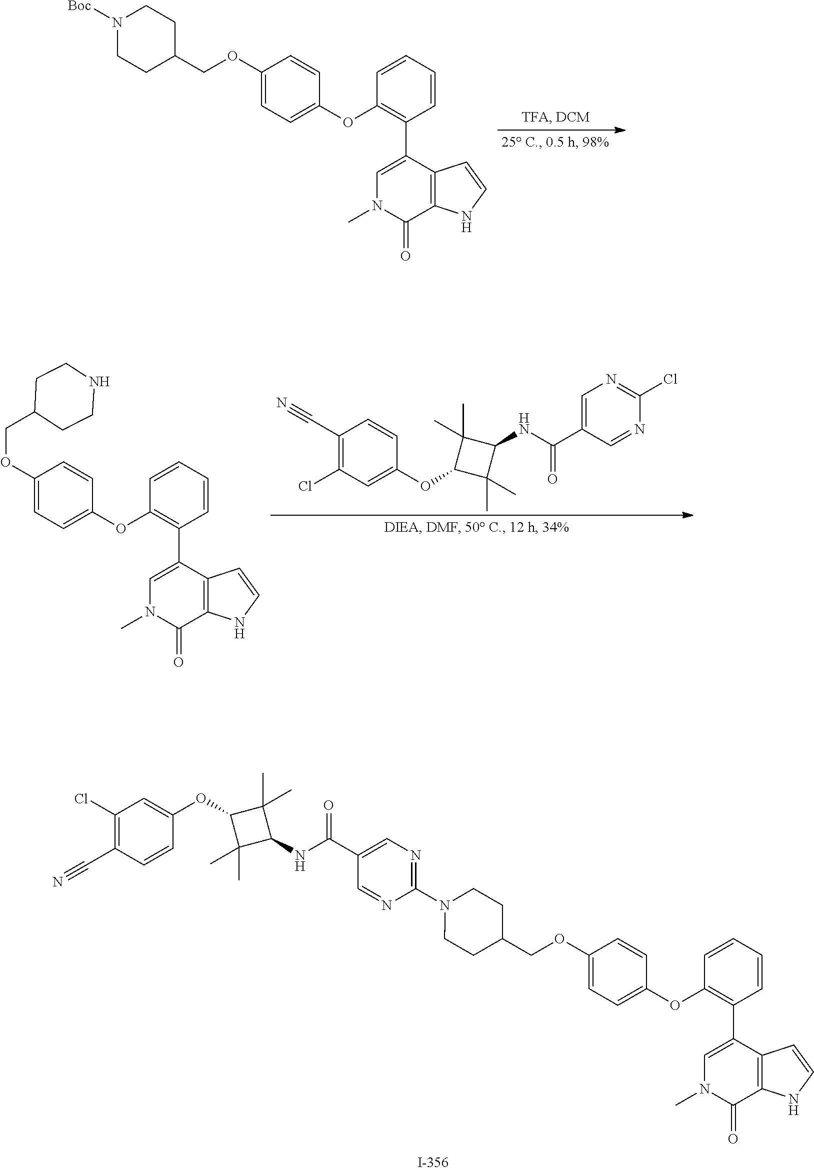 [SMILES: Cn1cc(-c2ccccc2Oc2ccc(OCC3CCN(C(=O)OC(C)(C)C)CC3)cc2)c2cc[nH]c2c1=O ; Cn1cc(-c2ccccc2Oc2ccc(OCC3CCNCC3)cc2)c2cc[nH]c2c1=O ; CC1(C)[C@H](NC(=O)c2cnc(Cl)nc2)C(C)(C)[C@H]1Oc1ccc(C#N)c(Cl)c1 ; Cn1cc(-c2ccccc2Oc2ccc(OCC3CCN(c4ncc(C(=O)N[C@H]5C(C)(C)[C@H](Oc6ccc(C#N)c(Cl)c6)C5(C)C)cn4)CC3)cc2)c2cc[nH]c2c1=O]

Step 1: Preparation of [4-(2-bromophenoxy)phenoxy]-tert-butyl-dimethyl-silane. The solution of 2-bromophenol (13 g, 75.14 mmol, 8.72 mL, 1 eq) and [4-[tert-butyl(dimethyl)silyl]oxyphenyl]boronic acid (22.74 g, 90.17 mmol, 1.2 eq) and Py (29.72 g, 375.71 mmol, 30.32 mL, 5 eq) and Cu(OAc)₂ (13.65 g, 75.14 mmol, 1 eq) and DIEA (48.56 g, 375.71 mmol, 65.44 mL, 5 eq) in DCM (150 mL) was stirred at 25° C. for 12 h under 02. TLC (petroleum ether: ethyl acetate=10:1) showed the reaction was completed. The mixture was filtered and the filtrate was concentrated under reduced pressure to get the residue. The crude product was purified by flash silica gel chromatography (ISCO®; 80 g SepaFlash® Silica Flash Column, Fluent of 0˜5% Ethylacetate/Petroleum ether gradient®70 mL/min). Compound [4-(2-bromophenoxy)phenoxy]-tert-butyl-dimethyl-silane (23 g, 60.63 mmol, 80.69% yield) was obtained as a white oil.

Step 2: Preparation of 4-(2-bromophenoxy)phenol. To a solution of [4-(2-bromophenoxy)phenoxy]-tert-butyl-dimethyl-silane (12 g, 31.63 mmol, 1 eq) was dissolved in HCl (1 M, 251.78 mL, 7.96 eq) in ethanol (120 mL). The mixture was stirred at 25° C. for 12 h. The reaction mixture was concentrated under reduced pressure to give a residue. The crude product was purified by flash silica gel chromatography (ISCO®; 80 g SepaFlash® Silica Flash Column, Fluent of 0˜5% Ethylacetate/Petroleum ether gradient a 70 mL/min). Compound 4-(2-bromophenoxy)phenol (δ g, 22.63 mmol, 71.55% yield) was obtained as a white solid.

Step 3: Preparation of tert-butyl 4-[[4-(2-bromophenoxy)phenoxy]methyl]piperidine-1-carboxylate. A mixture of 4-(2-bromophenoxy)phenol (1 g, 3.77 mmol, 1 eq), Cs₂CO₃ (3.69 g, 11.32 mmol, 3 eq), KI (626.18 mg, 3.77 mmol, 1 eq) and tert-butyl 4-(bromomethyl)piperidine-1-carboxylate (3.15 g, 11.32 mmol, 3 eq) in MeCN (20 mL) was degassed and purged with N₂ for 3 times, and then the mixture was stirred at 80° C. for 12 h under N₂ atmosphere.

The reaction mixture was filtered and concentrated under reduced pressure to give a residue. The crude product was purified by flash silica gel chromatography (ISCOn; 12 g SepaFlash® Silica Flash Column, Eluent of 0-5% Ethylacetate/Petroleum ether gradient®70 mL/min). Compound tert-butyl 4-[[4-(2-bromophenoxy)phenoxy]methyl]piperidine-1-carboxylate (1.5 g, 3.24 mmol, 86.00% yield) was obtained as a white solid.

Step 4: Preparation of tert-butyl4-[[4-[2-[6-methyl-7-oxo-1-(p-tolylsulfonyl)pyrrolo[2,3-c]pyridin-4-yl]phenoxy]phenoxy]methyl]piperidine-1-carboxylate. To a mixture of tert-butyl 4-[[4-(2-bromophenoxy)phenoxy]methyl]piperidine-1-carboxylate (200 mg, 432.55 mol, 1 eq), 6-methyl-1-(p-tolylsulfonyl)-4-(4,4,5,5-tetramethyl-1,3,2-dioxaborolan-2-yl)pyrrolo[2,3-c]pyridin-7-one (222.32 mg. 519.06 μmol, 1.2 eq) and SPhos Pd G₃ (67.50 mg, 86.51 mol, 0.2 eq) in THF (5 mL) was dropwise added a solution of K₃PO₄ (275.45 mg, 1.30 mmol, 3 eq) in H₂O (1 mL) under N₂ The reaction mixture was stirred at 60° C. for 12 h under N₂. To the reaction mixture was added water (20 mL) and the mixture was extracted with ethyl acetate (30 mL×3). The combined organic phase was washed with brine (20 mL), dried over anhydrous Na₂SO₄, filtered and concentrated in vacuo. The crude product was purified by flash silica gel chromatography (ISCO®; 20 g SepaFlash® Silica Flash Column, Eluent of 0˜60% Ethylacetate/Petroleum ether gradient®70 mL/min). Compound tert-butyl4-[[4-[2-[6-methyl-7-oxo-1-(p-tolyl sulfonyl)pyrrolo[2,3-c]pyridin-4-yl]phenoxy]phenoxy]methyl]piperidine-1-carboxylate (280 mg, 409.47 μmol, 94.66% yield) was obtained as a light yellow solid.

Step 5: Preparation of tert-butyl 4-[[4-[2-(6-methyl-7-oxo-1H-pyrrolo[2,3-c]pyridin-4-yl)phenoxy]phenoxy]methyl]piperidine-1-carboxylate. To a solution of tert-butyl 4-[[4-[2-[6-methyl-7-oxo-1-(p-tolylsulfonyl)pyrrolo[2,3-c]pyridin-4-yl]phenoxy]phenoxy]methyl]piperidine-1-carboxylate (280 mg, 409.47 μmol, 1 eq) in MeOH (10 mL) was added KOH (2 M, 2.66 mL, 13 eq). The mixture was stirred at 25° C. for 0.5 h. To the reaction mixture was added water (20 mL) and the mixture was extracted with ethyl acetate (30 mL×3). The combined organic phase was washed with brine (20 mL), dried over anhydrous Na₂SO₄, filtered and concentrated in vacuo. The residue was purified by prep-TLC (SiO₂, DCM: MeOH=10:1). Compound tert-butyl 4-[[4-[2-(6-methyl-7-oxo-1H-pyrrolo[2,3-c]pyridin-4-yl)phenoxy]phenoxy]methyl]piperidine-1-carboxylate (215 mg, 405.95 mol, 99.14% yield) was obtained as a light yellow solid.

Step 6: Preparation of 6-methyl-4-[2-[4-(4-piperidylmethoxy)phenoxy]phenyl]-1H-pyrrolo[2,3-c]pyridin-7-one. To a solution of tert-butyl 4-[[4-[2-(6-methyl-7-oxo-1H-pyrrolo[2,3-c]pyridin-4-yl)phenoxy]phenoxy]methyl]piperidine-1-carboxylate (100 mg, 188.81 mol, 1 eq) in DCM (10 mL) was added TFA (21.53 mg, 188.81 mol, 14.03 L., 1 eq). The mixture was stirred at 25° C. for 0.5 h. The reaction mixture was concentrated under reduced pressure to give a residue. The residue was taken to the next step without purification. Compound 6-methyl-4-[2-[4-(4-piperidy]methoxy)phenoxylphenyl]-1H-pyrrolo[2,3-c]pyridine-7-one (80 mg, 186.26 mol, 98.65% yield) was obtained as a yellow solid.

Step 7: Preparation of N-[3-(3-chloro-4-cyano-phenoxy)-2,2,4,4-tetramethyl-cyclobutyl]-2-[4-[[4-[2-(6-methyl-7-oxo-1H-pyrrolo[2,3-c]pyridin-4-yl)phenoxy]phenoxy]methyl]-1-piperidyl]pyrimidine-5-carboxamide. To A mixture of 6-methyl-4-[2-[4-(4-piperidylmethoxy)phenoxy]phenyl]-1H-pyrrolo[2,3-c]pyridin-7-one (80 mg, 186.26 mol, 1 eq), DIEA (481.45 mg, 3.73 mmol, 648.86 VL, 20 eq) and 2-chloro-N-[3-(3-chloro-4-cyano-phenoxy)-2,2,4,4-tetramethyl-cyclobutyl]pyrimidine-5-carboxamide (85 mg, 202.72 mol, 1.09 eq) in DMF (10 mL) was degassed and purged with N₂ for 3 times, and then the mixture was stirred at 50 C for 12 h under N₂ atmosphere. To the reaction mixture was added water (20 mL) and the mixture was extracted with ethyl acetate (30 mL×3). The combined organic phase was washed with brine (20 mL), dried over anhydrous Na₂SO₄, filtered and concentrated in vacuo. The crude product was purified by prep-HPLC (column: C18 150×30 mm;mobile phase: [water(FA)-ACN];gradient:70%-100% B over 7 min). Compound N-[3-(3-chloro-4-cyano-phenoxy)-2,2,4,4-tetramethyl-cyclobutyl]-2-[4-[[4-[2-(6-methyl-7-oxo-1H-pyrrolo[2,3-c]pyridin-4-yl)phenoxy]phenoxy]methyl]-1-piperidyl]pyrimidine-5-carboxamide (51.84 mg, 63.81 μmol, 34.26% yield) was obtained as a white solid. ¹H NMR (400 MHz, DMSO-d₆):δ 12.00 (br s, 1H), 8.76 (s, 2H), 7.91 (d, J=8.8 Hz, 1H), 7.72 (d, J=9.4 Hz, 1H), 7.46 (dd, J=1.7, 7.7 Hz, 1H), 7.33 (dt, J=1.7, 7.8 Hz, 1H), 7.27-7.25 (m, 2H), 7.22 (d, J=2.4 Hz, 1H), 7.20-7.15 (m, 1H), 7.01 (dd, J=2.3, 8.8 Hz, 1H), 6.91-6.84 (m, 5H), 6.23 (t, J=2.4 Hz, 1H), 4.79 (br d, J=12.6 Hz, 2H), 4.30 (s, 1H), 4.04 (d, J=9.4 Hz, 1H), 3.81 (d, J=6.1 Hz, 2H), 3.54 (s, 3H), 3.07-2.95 (m, 2H), 2.69-2.67 (m, 1H), 2.35-2.32 (m, 1H), 1.93-1.82 (m, 2H), 1.22 (s, 7H), 1.12 (s, 6H). LC-MS: MS (ES⁺): RT=0.689 min, m/z=812.4 [M+H⁺]; LCMS method: 5-95AB_R_220&254.1 cm

### Example 61—Synthesis of N-[3-(3-chloro-4-cyano-phenoxy)-2,2,4,4-tetramethyl-cyclobutyl]-2-[6-[2-[4-[4-(1-hydroxy-1-methyl-ethyl)-2-(6-methyl-7-oxo-1H-pyrrolo[2,3-c]pyridin-4-yl)phenoxy]phenyl]ethynyl]-2-azaspiro[3.3]heptan-2-yl]pyrimidine-5-carboxamide (I-357) o

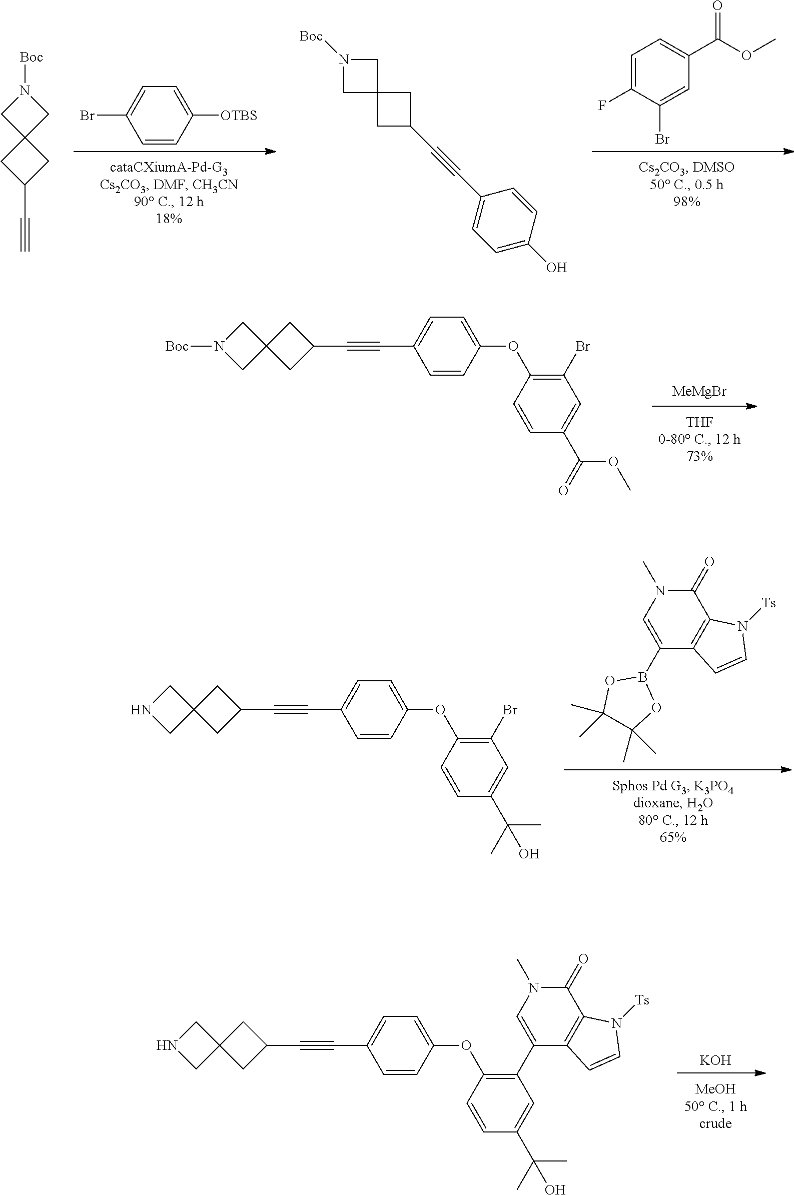

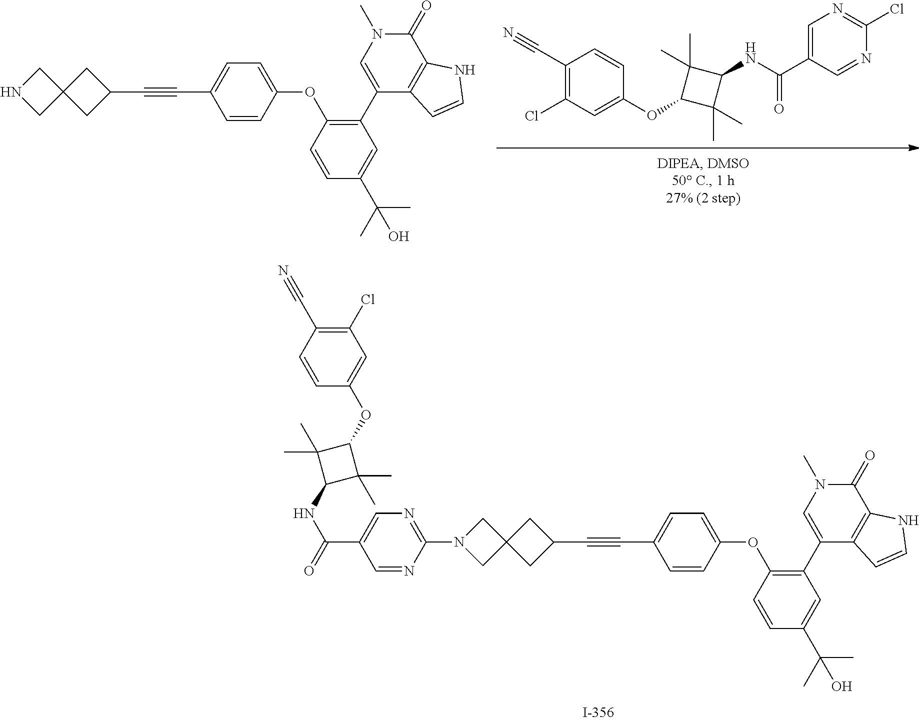 [SMILES: Cn1cc(-c2cc(C(C)(C)O)ccc2Oc2ccc(C#CC3CC4(CNC4)C3)cc2)c2cc[nH]c2c1=O ; CC1(C)[C@H](NC(=O)c2cnc(Cl)nc2)C(C)(C)[C@H]1Oc1ccc(C#N)c(Cl)c1 ; Cn1cc(-c2cc(C(C)(C)O)ccc2Oc2ccc(C#CC3CC4(C3)CN(c3ncc(C(=O)N[C@H]5C(C)(C)[C@H](Oc6ccc(C#N)c(Cl)c6)C5(C)C)cn3)C4)cc2)c2cc[nH]c2c1=O]

Step 1: Preparation of tert-butyl 6-[2-(4-hydroxyphenyl)ethynyl]-2-azaspiro[3.3]heptane-2-carboxylate. A mixture of tert-butyl 6-ethynyl-2-azaspiro [3.3]heptane-2-carboxylate (2.00 g, 9.04 mmol, 1 equiv), (4-bromophenoxy)-tert-butyl-dimethyl-silane (3.12 g, 10.9 mmol, 1.2 equiv), [2-(2-aminophenyl)phenyl]palladium (1+);bis(1-adamantyl)-butyl-phosphane;methanesulfonate (0.66 g, 0.90 mmol, 0.1 equiv) and Cs₂CO₃ (8.83 g, 27.1 mmol, 3 equiv) in DMF (30 mL) and MeCN (10 mL) was stirred at 90° C. for 12 h under N₂ atmosphere. The reaction mixture was concentrated in vacuo. The residue was purified by prep-HPLC (FA condition; column: Phenomenex luna C18 150*40 mm* 15 um;mobile phase: [water(FA)-ACN];gradient:47%-77% B over 15 min) to afford tert-butyl 6-[2-(4-hydroxyphenyl)ethynyl]-2-azaspiro[3.3]heptane-2-carboxylate (0.52 g, 1.66 mmol, 18% yield) as a white solid.

Step 2: Preparation of tert-butyl 6-[2-[4-(2-bromo-4-methoxycarbonyl-phenoxy)phenyl]ethynyl]-2-azaspiro[3.3]heptane-2-carboxylate. To a solution of tert-butyl 6-[2-(4-hydroxyphenyl)ethynyl]-2-azaspiro[3.3]heptane-2-carboxylate (0.52 g, 1.66 mmol, 1 equiv) and methyl 3-bromo-4-fluoro-benzoate (0.39 g, 1.66 mmol, 1 equiv) in DMSO (δ mL) was added Cs₂CO₃ (1.62 g, 4.98 mmol, 3 equiv) at 25° C. The mixture was stirred at 50° C. for 0.5 h. The reaction mixture was diluted with H₂O (25 mL) and extracted with ethyl acetate (20 mL*3). The combined organic layers were washed with brine (20 mL*3), dried with anhydrous Na₂SO₄, filtered and concentrated in vacuo. The residue was purified by column chromatography on silica gel (Petroleum ether/Ethyl acetate—5/1 to 3/1) to afford tert-butyl 6-[2-[4-(2-bromo-4-methoxy carbonyl-phenoxy)phenyl]ethynyl]-2-azaspiro [3.3]heptane-2-carboxylate (0.86 g, 1.63 mmol, 98% yield) as a colorless oil.

Step 3: Preparation of 2-[4-[4-[2-(2-azaspiro[3.3]heptan-6-yl)ethynyl]phenoxy]-3-bromo-phenyl]propan-2-ol. To a solution of tert-butyl 6-[2-[4-(2-bromo-4-methoxycarbonyl-phenoxy)phenyl]ethynyl]-2-azaspiro[3.3]heptane-2-carboxylate (500 mg, 0.95 mmol, 1 equiv) in THF (20 mL) was added dropwise MeMgBr (3 M, 6.33 mL, 20 equiv) at 0° C. The resulting mixture was stirred at 80° C. for 12 h. The reaction mixture was poured into water (20 mL). The aqueous phase was extracted with CH₂Cl₂ (10 mL*3). The combined organic phase was washed with brine (10 mL*3), dried with anhydrous Na₂SO₄, filtered and concentrated in vacuo. The residue was purified by column chromatography on silica gel (Dichloromethane/Methanol=20/1 to 10/1) to afford 2-[4-[4-[2-(2-azaspiro[3.3]heptan-6-yl)ethynyl]phenoxy]-3-bromo-phenyl]propan-2-ol (297 mg, 0.70 mmol, 73% yield) as a white solid.

Step 4: Preparation of 4-[2-[4-[2-(2-azaspiro[3.3]heptan-6-yl)ethynyl]phenoxy]-5-(1-hydroxy-1-methyl-ethyl)phenyl]-6-methyl-1-(p-tolylsulfonyl)pyrrolo[2,3-c]pyridin-7-one. A mixture of 2-[4-[4-[2-(2-azaspiro[3.3]heptan-6-yl)ethynyl]phenoxy]-3-bromo-phenyl]propan-2-ol (150 mg, 0.35 mmol, 1 equiv), 6-methyl-1-(p-tolylsulfonyl)-4-(4,4,5,5-tetramethyl-1,3,2-dioxaborolan-2-yl)pyrrolo[2,3-c]pyridin-7-one (181 mg, 0.42 mmol, 1.2 equiv), SPhos Pd G3 (27.5 mg, 0.04 mmol, 0.1 equiv) and K₃PO₄ (224 mg, 1.06 mmol, 3 equiv) in dioxane (3 mL) and H₂O (0.3 mL) was degassed and purged with N₂ for 3 times, and then the mixture was stirred at 80° C. for 12 h under N₂ atmosphere. The reaction mixture was concentrated in vacuo. The residue was purified by column chromatography on silica gel (Dichloromethane/Methanol=20/1 to 10/1) to afford 4-[2-[4-[2-(2-azaspiro[3.3]heptan-6-yl)ethynyl]phenoxy]-5-(1-hydroxy-1-methyl-ethyl)phenyl]-6-methyl-1-(p-tolylsulfonyl)pyrrolo[2,3-c]pyridin-7-one (150 mg, 0.23 mmol, 65% yield) as a yellow solid.

Step 5: Preparation of 4-[2-[4-[2-(2-azaspiro[3.3]heptan-6-yl)ethynyl]phenoxy]-5-(1-hydroxy-1-methyl-ethyl)phenyl]-6-methyl-1H-pyrrolo[2,3-c]pyridin-7-one. To a solution of 4-[2-[4-[2-(2-azaspiro[3.3]heptan-6-yl)ethynyl]phenoxy]-5-(1-hydroxy-1-methyl-ethyl)phenyl]-6-methyl-1-(p-tolylsulfonyl)pyrrolo[2,3-c]pyridin-7-one (140 mg, 0.22 mmol, 1 equiv) in MeOH (3 mL) was added KOH (60.6 mg, 1.08 mmol, 5 equiv) at 25° C. The mixture was stirred at 50° C. for 1 h. The reaction mixture was poured into water (10 mL). The aqueous phase was extracted with ethyl acetate (10 mL*3). The combined organic phase was washed with brine (10 mL*3), dried with anhydrous Na₂SO₄, filtered and concentrated in vacuo to afford 4-[2-[4-[2-(2-azaspiro[3.3]heptan-6-yl)ethynyl]phenoxy]-5-(1-hydroxy-1-methyl-ethyl)phenyl]-6-methyl-1H-pyrrolo[2,3-c]pyridin-7-one (100 mg, crude) as a yellow solid.

Step 6: Preparation of N-[3-(3-chloro-4-cyano-phenoxy)-2,2,4,4-tetramethyl-cyclobutyl]-2-[6-[2-[4-[4-(1-hydroxy-1-methyl-ethyl)-2-(6-methyl-7-oxo-1H-pyrrolo[2,3-c]pyridin-4-yl)phenoxy]phenyl]ethynyl]-2-azaspiro[3.3]heptan-2-yl]pyrimidine-5-carboxamide. To a solution of 4-[2-[4-[2-(2-azaspiro[3.3]heptan-6-yl)ethynyl]phenoxy]-5-(1-hydroxy-1-methyl-ethyl)phenyl]-6-methyl-1H-pyrrolo[2,3-c]pyridin-7-one (100 mg, 0.20 mmol, 1 equiv) and 2-chloro-N-[3-(3-chloro-4-cyano-phenoxy)-2,2,4,4-tetramethyl-cyclobutyl]pyrimidine-5-carboxamide (59.5 mg, 0.14 mmol, 0.7 equiv) in DMSO (3 mL) was added DIPEA (78.6 mg, 0.61 mmol, 0.11 mL, 3 equiv) at 25° C. The mixture was stirred at 50° C. for 1 h. The reaction mixture was diluted with H₂O(15 mL) and extracted with ethyl acetate (10 mL*3). The combined organic layers were washed with brine (10 mL*3), dried over anhydrous Na₂SO₄, filtered and concentrated in vacuo. The residue was purified by prep-HPLC (FA condition; column: Phenomenex luna C18 150*25 mm*0 um;mobile phase: [water(FA)-ACN];gradient:68%-88% B over 10 min) to afford N-[3-(3-chloro-4-cyano-phenoxy)-2,2,4,4-tetramethyl-cyclobutyl]-2-[6-[2-[4-[4-(1-hydroxy-1-methyl-ethyl)-2-(6-methyl-7-oxo-1H-pyrrolo[2,3-c]pyridin-4-yl)phenoxy]phenyl]ethynyl]-2-azaspiro[3.3]heptan-2-yl]pyrimidine-5-carboxamide (48.6 mg, 0.06 mmol, 27% yield, 99% purity) as a white solid. ¹H NMR (400 MHz, CD₃OD) δ 8.71 (s, 2H), 7.74-7.66 (m, 2H), 7.57-7.51 (m, 1H), 7.35-7.30 (m, 1H), 7.18-7.07 (m, 5H), 7.00-6.94 (m, 1H), 6.70-6.61 (m, 2H), 6.37-6.31 (m, 1H), 4.26 (s, 1H), 4.20 (d, 4H, J=8.0 Hz), 4.13 (s, 1H), 3.60 (s, 3H), 3.19-3.08 (m, 1H), 2.72-2.56 (m, 2H), 2.43-2.31 (m, 2H), 1.60 (s, 6H), 1.27 (s, 6H), 1.21 (s, 6H). LC-MS: MS (ES⁺): RT=2.851 min, m/z=876.4 [M+H⁺]; LCMS Method: 25.

### Example 62—Synthesis of N-[3-[[1-[6-[[3-(3-chloro-4-cyano-phenoxy)-2,2,4,4-tetramethyl-cyclobutyl]carbamoyl]-3-pyridyl]-4-piperidyl]methyl]cyclobutyl]-4-[5-(1-hydroxy-1-methyl-ethyl)-2-phenoxy-phenyl]-6-methyl-7-oxo-1H-pyrrolo[2,3-c]pyridine-2-carboxamide (I-377) 0 HO

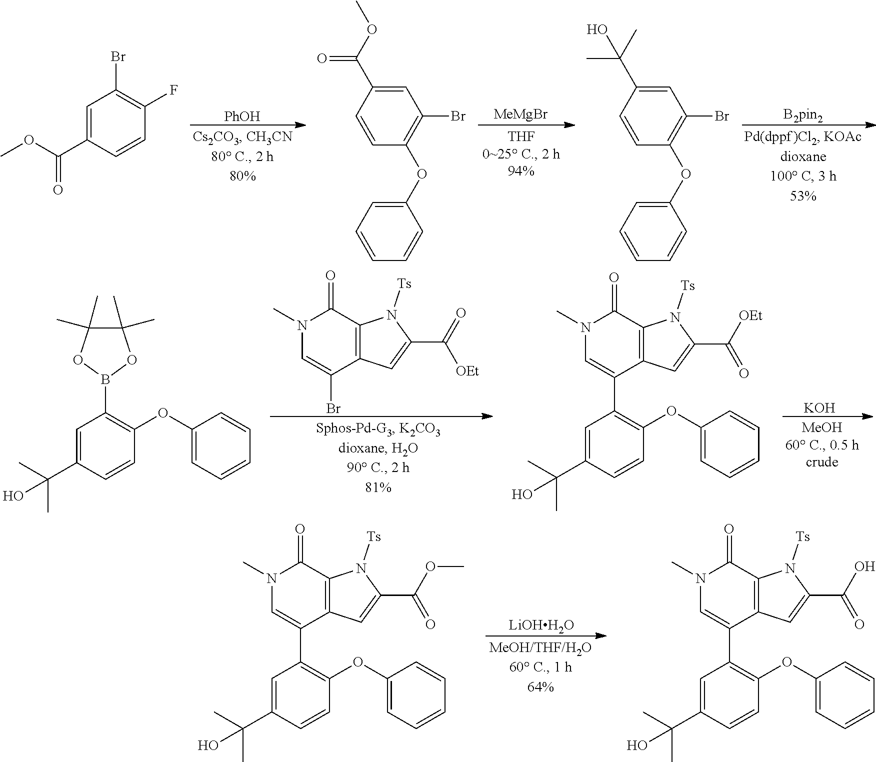

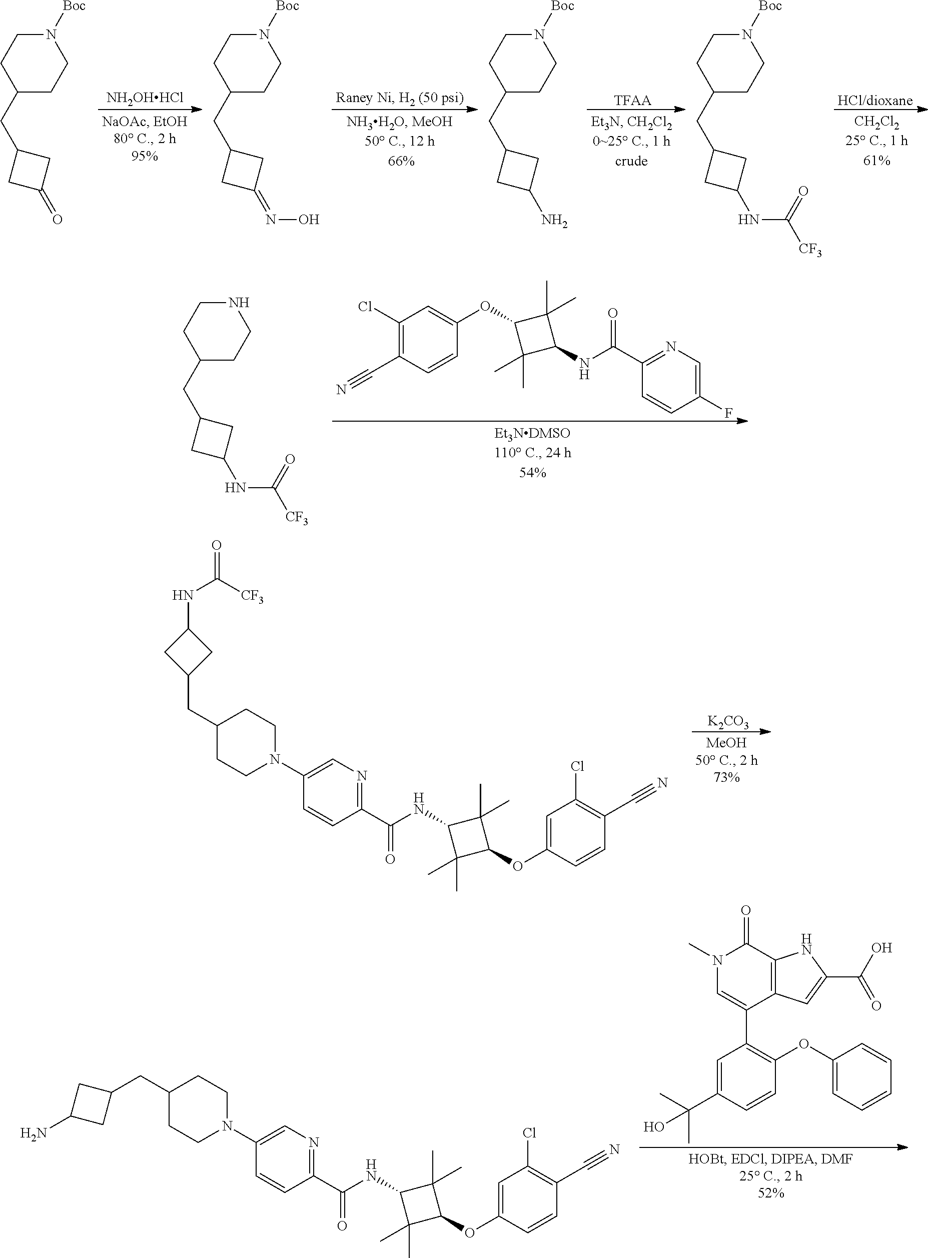

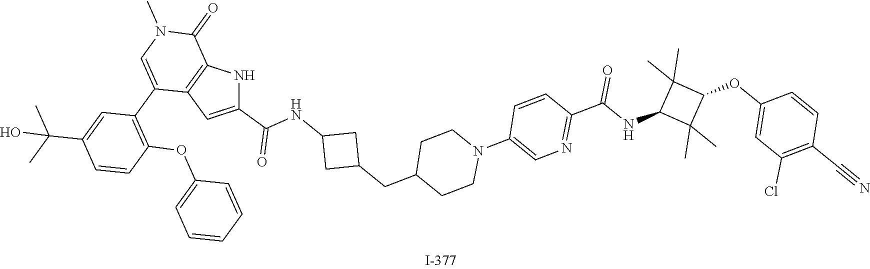 [SMILES: Cn1cc(-c2cc(C(C)(C)O)ccc2Oc2ccccc2)c2cc(C(=O)NC3CC(CC4CCN(c5ccc(C(=O)N[C@H]6C(C)(C)[C@H](Oc7ccc(C#N)c(Cl)c7)C6(C)C)nc5)CC4)C3)[nH]c2c1=O]

Step 1: Preparation of methyl 3-bromo-4-phenoxy-benzoate. To a mixture of methyl 3-bromo-4-fluoro-benzoate (3.00 g, 12.9 mmol, 1 equiv) and phenol (1.21 g, 12.9 mmol, 1.13 mL, 1 equiv) in CH₃CN (30 mL) was added Cs₂CO₃ (8.39 g, 25.75 mmol, 2 equiv) at 25° C. The reaction mixture was stirred at 80° C. for 2 h. The reaction mixture was cooled to 25° C. The reaction mixture was diluted with H₂O (50 mL). The resulting mixture was extracted with ethyl acetate (30 mL×3). The combined organic phases were washed with brine (30 mL), dried with anhydrous Na₂SO₄, filtered and concentrated in vacuo. The residue was purified by column chromatography by silica gel (Petroleum ether/Ethyl acetate=20/1) to afford methyl 3-bromo-4-phenoxy -benzoate (3.16 g, 10.3 mmol, 80% yield) as a colorless oil.

Step 2: Preparation of 2-(3-bromo-4-phenoxy-phenyl)propan-2-ol. To a mixture of methyl 3-bromo-4-phenoxy-benzoate (3.00 g, 9.77 mmol, 1 equiv) in THF (50 mL) was slowly added dropwise MeMgBr (3 M, 9.77 mL, 3 equiv) at 0° C. The reaction mixture was stirred at 25° C. for 2 h under N₂ atmosphere. The reaction mixture was slowly quenched by addition to saturated aqueous solution NH₄Cl (150 mL) at 0° C. Then the resulting mixture was extracted with ethyl acetate (30 mL×3). The combined organic phases were washed with brine (30 mL), dried with anhydrous Na₂SO₄, filtered and concentrated in vacuo. The residue was purified by column chromatography by silica gel (Petroleum ether/Ethyl acetate=8/1) to afford 2-(3-bromo-4-phenoxy-phenyl)propan-2-ol (2.82 g, 9.18 mmol, 94% yield) as a light yellow oil.

Step 3: Preparation of 2-[4-phenoxy-3-(4,4,5,5-tetramethyl-1,3,2-dioxaborolan-2-yl)phenyl]propan-2-ol. To a mixture of 2-(3-bromo-4-phenoxy-phenyl)propan-2-ol (1.70 g, 5.53 mmol, 1 equiv) and BPD (1.69 g, 6.64 mmol, 1.2 equiv) in dioxane (20 mL) was added KOAc (1.63 g, 16.6 mmol, 3 equiv) and Pd(dppf)Cl₂ (0.41 g, 0.55 mmol, 0.1 equiv) at 25° C. The reaction mixture was stirred at 100° C. for 3 h under N₂ atmosphere. The reaction mixture was cooled to 25° C. The reaction mixture was filtered, the residue was washed with ethyl acetate (20 mL×3). The combined organic phase was concentrated in vacuo. The residue was purified by column chromatography by silica gel (Petroleum ether/Ethyl acetate=9/1) to afford 2-[4-phenoxy-3-(4,4,5,5-tetramethyl-1,3,2-dioxaborolan-2-yl)phenyl]propan-2-ol (0.96 g, 2.71 mmol, 53% yield) as a light yellow oil.

Step 4: Preparation of Ethyl 4-[5-(1-hydroxy-1-methyl-ethyl)-2-phenoxy-phenyl]-6-methyl-7-oxo-1-(p-tolylsulfonyl)pyrrolo[2,3-c]pyridine-2-carboxylate. To a mixture of 2-[4-phenoxy-3-(4,4,5,5-tetramethyl-1,3,2-dioxaborolan-2-yl)phenyl]propan-2-ol (150 mg, 0.42 mmol, 1 equiv) and ethyl 4-bromo-6-methyl-7-oxo-1-(p-tolylsulfonyl) pyrrolo[2,3-c]pyridine-2-carboxylate (154 mg, 0.34 mmol, 0.8 equiv) in dioxane (5 mL) and H₂O(0.5 mL) was added SPhos Pd G3 (33.0 mg, 0.04 mmol, 0.1 equiv) and K₂CO₃ (117 mg, 0.85 mmol, 2 equiv) at 25° C. The reaction mixture was stirred at 90° C. for 2 h under N₂ atmosphere. The reaction mixture was cooled to 25° C. The reaction mixture was diluted with H₂O (30 mL) and extracted with ethyl acetate (20 mL×3). The combined organic phases were washed with brine (20 mL), dried with anhydrous Na₂SO₄, filtered and concentrated in vacuo. The residue was purified by column chromatography by silica gel (Petroleum ether/Ethyl acetate=1/1) to afford ethyl 4-[5-(1-hydroxy-1-methyl-ethyl)-2-phenoxy-phenyl]-6-methyl-7-oxo-1-(p-tolylsulfonyl)pyrrolo[2,3-c]pyridine-2-carboxylate (205 mg, 0.34 mmol, 81% yield) as a light yellow oil.

Step 5: Preparation of Methyl 4-[5-(1-hydroxy-1-methyl-ethyl)-2-phenoxy-phenyl]-6-methyl-7-oxo-1H-pyrrolo[2,3-c]pyridine-2-carboxylate. To a mixture of ethyl 4-[5-(1-hydroxy-1-methyl-ethyl)-2-phenoxy-phenyl]-6-methyl-7-oxo-1-(ptolylsulfonyl)pyrrolo[2,3-c]pyridine-2-carboxylate (200 mg, 0.33 mmol, 1 equiv) in MeOH (5 mL) was added KOH (187 mg, 3.33 mmol, 10 equiv) at 25° C. The reaction mixture was stirred at 60° C. for 0.5 h. The reaction mixture was cooled to 25° C. The reaction mixture was diluted with H₂O (30 mL) and extracted with ethyl acetate (20 mL×3). The combined organic phases were washed with brine (20 mL), dried with anhydrous Na₂SO₄, filtered and concentrated in vacuo to afford methyl 4-[5-(1-hydroxy-1-methyl-ethyl)-2-phenoxy-phenyl]-6-methyl-7-oxo-1H-pyrrolo[2,3-c]pyridine-2-carboxylate (130 mg, crude) as a light yellow oil.

Step 6: Preparation of 4-[5-(1-hydroxy-1-methyl-ethyl)-2-phenoxy-phenyl]-6-methyl-7-oxo-1H-pyrrolo[2,3-c]pyridine-2-carboxylic acid. To a mixture of methyl 4-[5-(1-hydroxy-1-methyl-ethyl)-2-phenoxy-phenyl]-6-methyl-7-oxo-1H-pyrrolo[2,3-c]pyridine-2-carboxylate (125 mg, 0.29 mmol, 1 equiv) in MeOH (1 mL), THF (1 mL) and H₂O (1 mL) was added LiOH H₂O (60.7 mg, 1.45 mmol, 5 equiv) at 25° C. The reaction mixture was stirred at 60° C. for 1 h. The reaction mixture was concentrated in vacuo. The residue was diluted with H₂O (30 mL). The pH of the mixture was adjusted to 7 by addition of HCl (1N) solution. The resulting mixture was extracted with ethyl acetate (20 mL×3). The combined organic phases were washed with brine (20 mL), dried with anhydrous Na₂SO₄, filtered and concentrated in vacuo. The residue was purified by prep-HPLC (column: Phenomenex luna C18 150*25 mm* 10 um; mobile phase: [water (FA)-ACN]; gradient: 30%-60% B over 9 min) to afford 4-[5-(1-hydroxy-1-methyl-ethyl)-2-phenoxy-phenyl]-6-methyl-7-oxo-1H-pyrrolo[2,3-c]pyridine-2-carboxylic acid (77 mg, 0.18 mmol, 64% yield) as a light yellow solid.

Step 7: Preparation of tert-butyl 4-[(3-hydroxyiminocyclobutyl)methyl]piperidine-1-carboxylate. To a mixture of tert-butyl 4-[(3-oxocyclobutyl)methyl]piperidine-1-carboxylate (2.00 g, 7.48 mmol, 1 equiv) and NH₂OH HCl (2.60 g, 37.4 mmol, 5 equiv) in EtOH (20 mL) was added NaOAc (3.07 g, 37.4 mmol, 5 equiv) at 25° C. The reaction mixture was stirred at 80° C. for 2 h under N₂ atmosphere. The reaction mixture was cooled to 25° C. The reaction mixture was diluted with H₂O (50 mL). The resulting mixture was extracted with ethyl acetate (30 mL×3). The combined organic phases were washed with brine (30 mL), dried with anhydrous Na₂SO₄, filtered and concentrated in vacuo. The residue was purified by column chromatography by silica gel (Petroleum ether/Ethyl acetate=3/1) to afford tert-butyl 4-[(3-hydroxyiminocyclobutyl)methyl]piperidine-1-carboxylate (2.00 g, 7.08 mmol, 95% yield) as a light yellow solid.

Step 8: Preparation of tert-butyl 4-[(3-aminocyclobutyl)methyl]piperidine-1-carboxylate. To a mixture of tert-butyl 4-[(3-hydroxyiminocyclobutyl)methyl]piperidine-1-carboxylate (2.00 g, 7.08 mmol, 1 equiv) and NH₃—H₂O (9.10 g, 64.9 mmol, 10 mL, 25% purity, 9.17 equiv) in MeOH (40 mL) was slowly added Raney-Ni (200 mg) at 25° C. The suspension was degasses under vacuum and purged with H₂ several times. The reaction mixture was stirred at 50° C. for 12 h under H₂ (50 Psi) atmosphere. The reaction mixture was filtered and the filtrate was concentrated in vacuo. The residue was purified by prep-HPLC (column: Waters Xbridge C18 150*50 mm* 10 um; mobile phase: [water (NH₄HCO₃)-ACN]; gradient: 18%-48% B over 10 min) to afford tert-butyl 4-[(3-aminocyclobutyl) methyl]piperidine-1-carboxylate (1.26 g, 4.69 mmol, 66% yield) as a colorless oil.

Step 9: Preparation of tert-butyl 4-1[3-[(2,2,2-trifluoroacetyl)aμmolcyclobutyl]methyl]piperidine-1-carboxylate. To a mixture of tert-butyl 4-[(3-aminocyclobutyl)methyl]piperidine-1-carboxylate (500 mg, 1.86 mmol, 1 equiv) and Et₃N (754 mg, 7.45 mmol, 1.04 mL, 4 equiv) in CH₂Cl₂ (5 mL) was added TFAA (509 mg, 2.42 mmol, 0.34 mL, 1.3 equiv) at 0° C. The reaction mixture was stirred at 25° C. for 1 h. The reaction mixture was diluted with H₂O (30 mL) and the resulting mixture was extracted with CH₂Cl₂ (20 mL×3). The combined organic phases were washed with brine (20 mL), dried with anhydrous Na₂SO₄, filtered and concentrated in vacuo to afford tert-butyl 4-[[3-[(2,2,2-trifluoroacetyl)amino]cyclobutyl]methyl]piperidine-1-carboxylate (700 mg, crude) as a light yellow oil.

Step 10: Preparation of 2,2,2-trifluoro-N-[3-(4-piperidylmethyl)cyclobutyl]acetamide. To a mixture of tert-butyl 4-[[3-[(2,2,2-trifluoroacetyl)amino]cyclobutyl]methyl]piperidine-1-carboxylate (700 mg, 1.92 mmol, 1 equiv) in CH₂Cl₂ (5 mL) was added HCl/dioxane (4 M, 5 mL) at 25° C. The reaction mixture was stirred at 25° C. for 1 h. The reaction mixture was concentrated in vacuo. The residue was purified by prep-HPLC (column: Phenomenex Luna C18 150*25 mm*10 um; mobile phase: [water (HCl)-ACN]; gradient: 2%-32% B over 8 min) to afford 2,2,2-trifluoro-N-[3-(4-piperidylmethyl)cyclobutyl]acetamide (350 mg, 1.16 mmol, 61% yield, HCl salt) as a colorless oil.

Step 11: Preparation of N-[3-(3-chloro-4-cyano-phenoxy)-2,2,4,4-tetramethyl-cyclobutyl]-5-[4-[[3-[(2,2,2-trifluoroacetyl)amino]cyclobutyl]methyl]-1-piperidyl]pyridine-2-carboxamide. To a mixture of 2,2,2-trifluoro-N-[3-(4-piperidylmethyl) cyclobutyl]acetamide (150 mg, 0.50 mmol, 1 equiv, HCl salt) and N-[3-(3-chloro-4-cyano-phenoxy)-2,2,4,4-tetramethyl-cyclobutyl]-5-fluoro-pyridine-2-carboxamide (80.2 mg, 0.20 mmol, 0.4 equiv) in DMSO (1 mL) was added Et₃N (303 mg, 2.99 mmol, 0.42 mL, 6 equiv) at 25° C. The reaction mixture was stirred at 1100 C. for 24 h under N₂ atmosphere. The reaction mixture was concentrated in vacuo. The residue was purified by preparative HPLC (column: Phenomenex luna C18 150*40 mm* 15 um; mobile phase: [water (FA)-ACN]; gradient: 62%-92% B over 15 min) to afford N-[3-(3-chloro-4-cyano-phenoxy)-2,2,4,4-tetramethyl-cyclobutyl]-5-[4-[[3-[(2,2,2-trifluoroacetyl)amino]cyclobutyl]methyl]-1-piperidyl]pyridine-2-carboxamide (174 mg, 0.27 mmol, 54% yield) as a light yellow solid.

Step 12: Preparation of 5-[4-[(3-aminocyclobutyl)methyl]-1-piperidyl]-N-[3-(3-chloro-4-cyano-phenoxy)-2,2,4,4-tetramethylcyclobutyl]pyridine-2-carboxamide. To a mixture of N-[3-(3-chloro-4-cyano-phenoxy)-2,2,4,4-tetramethyl-cyclobutyl]-5-[4-[[3-[(2,2,2-trifluoroacetyl)amino]cyclobutyl]methyl]-1-piperidyl]pyridine-2-carboxamide (170 mg, 0.26 mmol, 1 equiv) in MeOH (5 mL) was added K₂CO₃ (182 mg, 1.32 mmol, 5 equiv) at 25° C. The reaction mixture was stirred at 50° C. for 2 h. The reaction mixture was diluted with ethyl acetate (20 mL) and water (20 mL). The resulting mixture was extracted with ethyl acetate (10 mL*3). The combined organic phase was dried with anhydrous Na₂SO₄, filtered and concentrated in vacuo. The residue was purified by preparative HPLC (column: Waters xbridge 150*25 mm 10 um; mobile phase: [water (NH₄HCO₃)-ACN]; gradient: 68%-88% B over 8 min) to afford 5-[4-[(3-aminocyclobutyl)methyl]-1-piperidyl]-N-[3-(3-chloro-4-cyano-phenoxy)-2,2,4,4-tetramethylcyclobutyl]pyridine-2-carboxamide (105 mg, 0.19 nmol, 73% yield) as a light yellow solid.

Step 13: Preparation of N-[3-[[1-[6-[[3-(3-chloro-4-cyano-phenoxy)-2,2,4,4-tetramethyl-cyclobutyl]carbamoyl]-3-pyridyl]-4-piperidyl]methyl]cyclobutyl]-4-[5-(1-hydroxy-1-methyl-ethyl)-2-phenoxy-phenyl]-6-methyl-7-oxo-1H-pyrrolo[2,3-c]pyridine-2-carboxamide. To a mixture of 5-[4-[(3-aminocyclobutyl)methyl]-1-piperidyl]-N-[3-(3-chloro-4-cyano-phenoxy)-2,2,4,4-tetramethyl-cyclobutyl]pyridine-2-carboxamide (90 mg, 0.16 mmol, 1 equiv) and 4-[5-(1-hydroxy-1-methyl-ethyl)-2-phenoxy-phenyl]-6-methyl-7-oxo-1H-pyrrolo[2,3-c]pyridine-2-carboxylic acid (68.5 mg, 0.16 mmol, 1 equiv) in DMF (2 mL) was added HOBt (44.2 mg, 0.33 mmol, 2 equiv), EDCI (62.7 mg, 0.33 mmol, 2 equiv) and DIPEA (63.4 mg, 0.49 mmol, 0.09 mL, 3 equiv) at 25° C. The reaction mixture was stirred at 25° C. for 2 h. The reaction mixture was diluted with H₂O (30 mL) and the resulting mixture was extracted with ethyl acetate (20 mL×3). The combined organic phases were washed with brine (20 mL), dried with anhydrous Na₂SO₄, filtered and concentrated in vacuo. The residue was purified by preparative HPLC (column: Phenomenex luna C18 150*25 mm* 10 um; mobile phase: [water (FA)-ACN]; gradient: 68%-98% B over 10 min). The residue was furthered purified by Chiral SFC (column: DAICEL CHIRALPAK AD (250 mm*30 mm, 0 um); mobile phase: [CO₂-ACN/i-PrOH (0.1% NH₃H₂O)]; B %: 65%, isocratic elution mode) to afford N-[3-[[1-[6-[[3-(3-chloro-4-cyano-phenoxy)-2,2,4,4-tetramethyl-cyclobutyl]carbamoyl]-3-pyridyl]-4-piperidyl]methyl]cyclobutyl]-4-[5-(1-hydroxy-1-methyl-ethyl)-2-phenoxy-phenyl]-6-methyl-7-oxo-1H-pyrrolo[2,3-c]pyridine-2-carboxamide (40.8 mg, 0.04 mmol, 26% yield) as a white solid. ¹H NMR (400 MHz, CDCl₃) δ 10.86-10.35 (m, 1H), 8.18-8.11 (m, 2H), 7.98 (d, 1H, J=8.8 Hz), 7.70 (d, 1H, J=2.4 Hz), 7.55 (d, 1H, J=8.8 Hz), 7.40-7.37 (m, 1H), 7.24-7.14 (m, 3H), 7.04-6.95 (m, 4H), 6.87-6.73 (m, 5H), 4.42-4.29 (m, 1H), 4.13-4.11 (m, 1H), 4.06 (s, 1H), 3.79-3.76 (m, 2H), 3.56 (s, 3H), 2.86-2.69 (m, 3H), 2.52-2.50 (m, 2H), 2.08-1.98 (m, 1H), 1.92 (s, 3H), 1.70-1.68 (m, 2H), 1.63 (s, 6H), 1.54-1.36 (m, 3H), 1.27 (s, 6H), 1.18 (s, 6H). LC-MS: RT=2.889 min, m/z=950.6 [M+H⁺]; LCMS Method: 25.

### Example 63—Synthesis of N-[3-(3-chloro-4-cyano-phenoxy)-2,2,4,4-tetramethyl-cyclobutyl]-2-[4-[[1-[2-[4-(1-hydroxy-1-methyl-ethyl)-2-(6-methyl-7-oxo-1H-pyrrolo[2,3-c]pyridin-4-yl)phenoxy]phenyl]azetidin-3-yl]methyl]-1-piperidyl]pyrimidine-5-carboxamide (1-387)

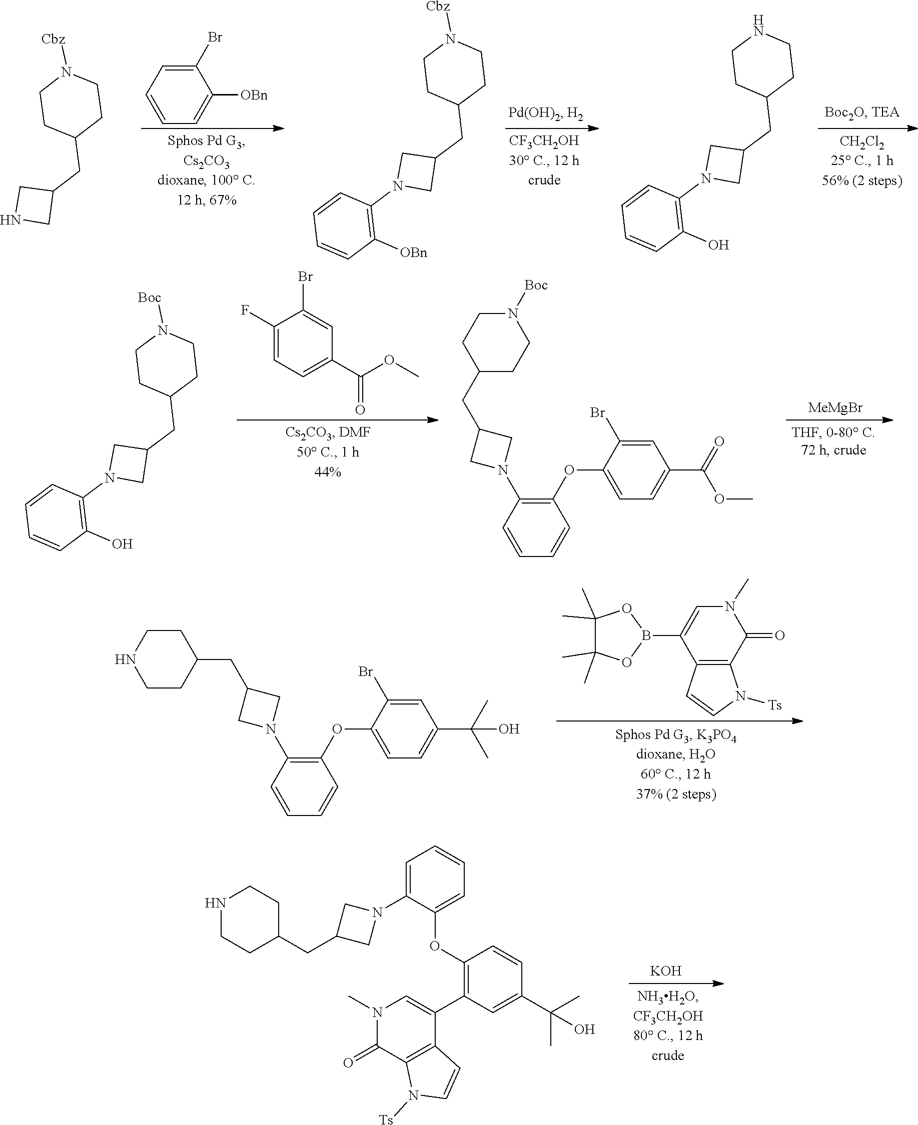

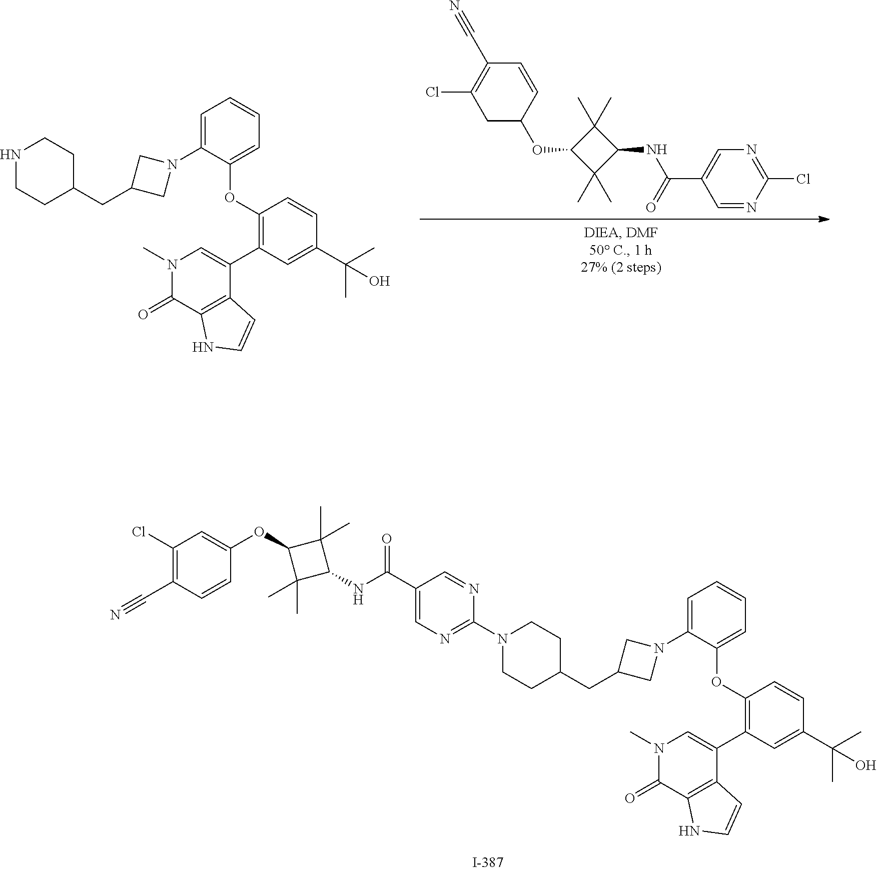 [SMILES: Cn1cc(-c2cc(C(C)(C)O)ccc2Oc2ccccc2N2CC(CC3CCNCC3)C2)c2cc[nH]c2c1=O ; CC1(C)[C@H](NC(=O)c2cnc(Cl)nc2)C(C)(C)[C@H]1OC1C=CC(C#N)=C(Cl)C1 ; Cn1cc(-c2cc(C(C)(C)O)ccc2Oc2ccccc2N2CC(CC3CCN(c4ncc(C(=O)N[C@H]5C(C)(C)[C@H](Oc6ccc(C#N)c(Cl)c6)C5(C)C)cn4)CC3)C2)c2cc[nH]c2c1=O]

Step 1: Preparation of Benzyl 4-[[1-(2-benzyloxyphenyl)azetidin-3-yl]methyl]piperidine-1-carboxylate. To a solution of benzyl 4-(azetidin-3-ylmethyl)piperidine-1-carboxylate (0.85 g, 2.95 mmol, 1 equiv) and 1-benzyloxy-2-bromo-benzene (1.16 g, 4.42 mmol, 0.83 mL, 1.5 equiv) in dioxane (8 mL) was added Cs₂CO₃ (2.88 g, 8.84 mmol, 3 equiv) and SPhos Pd G3 (0.46 g, 0.59 mmol, 0.2 equiv). The mixture was stirred at 100° C. for 12 h under N₂ atmosphere. The reaction mixture was filtered and concentrated in vacuo. The residue was purified by column chromatography on silica gel (Petroleum ether/Ethyl acetate=4/1 to 3/1) to afford benzyl 4-[[1-(2-benzyloxyphenyl)azetidin-3-yl]methyl]piperidine-1-carboxylate (0.93 g, 1.98 mmol, 67% yield) as a yellow oil.

Step 2: Preparation of 2-[3-(4-piperidylmethyl)azetidin-1-yl]phenol. To a solution of benzyl 4-[[1-(2-benzyloxyphenyl)azetidin-3-yl]methyl]piperidine-1-carboxylate (1.00 g, 2.12 mmol, 1 equiv) in CF₃CH₂OH (10 mL) was added Pd(OH)₂ (1 g) under N₂. The mixture was stirred at 30° C. for 12 h under H₂ (50 Psi). The reaction mixture was filtered and the filtrate was concentrated in vacuo to afford 2-[3-(4-piperidylmethyl)azetidin-1-yl]phenol (0.52 g, crude) as a brown oil.

Step 3: Preparation of tert-butyl 4-[[1-(2-hydroxyphenyl)azetidin-3-yl]methyl]piperidine-1-carboxylate. To a solution of 2-[3-(4-piperidylmethyl)azetidin-1-yl]phenol (523 mg, 2.12 mmol, 1 equiv) in CH₂Cl₂ (5 mL) was added TEA (430 mg, 4.25 mmol, 0.59 mL, 2 equiv) and tert-butoxycarbonyl tert-butyl carbonate (510 mg, 2.34 mmol, 0.54 mL, 1.1 equiv) at 25° C. The mixture was stirred at 25° C. for 1 h. The reaction mixture was diluted with water (5 mL) and extracted with CH₂Cl₂ (3 mL*3). The combined organic layers were dried with anhydrous Na₂SO₄, filtered and concentrated in vacuo. The residue was purified by column chromatography on silica gel (Petroleum ether/Ethyl acetate=4/1 to 3/1) to afford tert-butyl 4-[[1-(2-hydroxyphenyl)azetidin-3-yl]methyl]piperidine-1-carboxylate (410 mg, 1.18 mmol, 56% yield for 2 steps) as a yellow oil.

Step 4: Preparation of tert-butyl 4-[[1-[2-(2-bromo-4-methoxycarbonyl-phenoxy)phenyl]azetidin-3-yl]methyl]piperidine-1-carboxylate. To a solution of tert-butyl 4-[[1-(2-hydroxyphenyl)azetidin-3-yl]methyl]piperidine-1-carboxylate (0.41 g, 1.18 mmol, 1 equiv) in DMF (δ mL) was added Cs₂CO₃ (1.16 g, 3.55 mmol, 3 equiv) and methyl 3-bromo-4-fluoro-benzoate (0.30 g, 1.30 mmol, 1.1 equiv) at 25° C. The mixture was stirred at 50° C. for 1 h. The reaction mixture was diluted with H₂O (10 mL) and extracted with ethyl acetate (5 mL*3). The combined organic layers were washed with brine (5 mL*5), dried with anhydrous Na₂SO₄, filtered and concentrated in vacuo. The residue was purified by column chromatography on silica gel (Petroleum ether/Ethyl acetate=4/1 to 3/1) to afford tert-butyl 4-[[1-[2-(2-bromo-4-methoxycarbonyl-phenoxy)phenyl]azetidin-3-yl]methyl]piperidine-1-carboxylate (0.29 g, 0.52 mmol, 44% yield) as a yellow oil.

Step 5: Preparation of 2-[3-bromo-4-[2-[3-(4-piperidylmethyl)azetidin-1-yl]phenoxy]phenyl]propan-2-ol. To a solution of tert-butyl 4-[[1-[2-(2-bromo-4-methoxycarbonyl-phenoxy)phenyl]azetidin-3-yl]methyl]piperidine-1-carboxylate (290 mg, 0.52 mmol, 1 equiv) in THF (5 mL) was added dropwise bromo(methyl)magnesium (3 M, 2.59 mL, 15 equiv) at 0° C. under N₂. The mixture was stirred at 80° C. for 72 h under N₂ atmosphere. The reaction mixture was quenched with saturated aqueous NH₄Cl (10 mL) at 0° C., and then diluted with water (5 mL) and extracted with CH₂Cl₂ (5 mL*3). The combined organic layers were dried with anhydrous Na₂SO₄, filtered and concentrated in vacuo to afford 2-[3-bromo-4-[2-[3-(4-piperidylmethyl)azetidin-1-yl]phenoxy]phenyl]propan-2-ol (238 mg, crude) as a yellow solid.

Step 6: Preparation of 4-[5-(1-hydroxy-1-methyl-ethyl)-2-[2-[3-(4-piperidylmethyl)azetidin-1-yl]phenoxy]phenyl]-6-methyl-1-(p-tolylsulfonyl)pyrrolo[2,3-c]pyridin-7-one. To a solution of 2-[3-bromo-4-[2-[3-(4-piperidylmethyl)azetidin-1-yl]phenoxy]phenyl]propan-2-ol (170 mg, 0.37 mmol, 1 equiv) and 6-methyl-1-(p-tolylsulfonyl)-4-(4,4,5,5-tetramethyl-1,3,2-dioxaborolan-2-yl)pyrrolo[2,3-c]pyridin-7-one (190 mg, 0.44 mmol, 1.2 equiv) in dioxane (2 mL) and H₂O (0.2 mL) was added SPhos Pd G3 (28.9 mg, 0.04 mmol, 0.1 equiv) and K₃PO₄ (236 mg, 1.11 mmol, 3 equiv) at 25° C. The mixture was stirred at 60° C. for 12 h under N₂ atmosphere. The reaction mixture was filtered and concentrated in vacuo. The residue was purified by prep-TLC (Dichloromethane/Methanol=10/1) to afford 4-[5-(1-hydroxy-1-methyl-ethyl)-2-[2-[3-(4-piperidylmethyl)azetidin-1-yl]phenoxy]phenyl]-6-methyl-1-(p-tolylsulfonyl)pyrrolo[2,3-c]pyridin-7-one (94.0 mg, 0.14 mmol, 37% yield for 2 steps) as a yellow solid.

Step 7: Preparation of 4-[5-(1-hydroxy-1-methyl-ethyl)-2-[2-[3-(4-piperidylmethyl)azetidin-1-yl]phenoxy]phenyl]-6-methyl-1H-pyrrolo[2,3-c]pyridin-7-one. To a solution of 4-[5-(1-hydroxy-1-methyl-ethyl)-2-[2-[3-(4-piperidylmethyl)azetidin-1-yl]phenoxy]phenyl]-6-methyl-1-(p-tolylsulfonyl)pyrrolo[2,3-c]pyridin-7-one (94.0 mg, 0.14 mmol, 1 equiv) in CF₃CH₂OH (2 mL) was added KOH (38.7 mg, 0.69 mmol, 5 equiv) and NH₃.H₂O (91.0 mg, 0.26 mmol, 0.1 mL, 10% purity, 1.88 equiv) at 25° C. The mixture was stirred at 80° C. for 12 h. The reaction mixture was diluted with H₂O (10 mL) and extracted with ethyl acetate (8 mL*3). The combined organic layers were washed with brine (5 mL*3), dried with anhydrous Na₂SO₄, filtered and concentrated in vacuo to afford 4-[5-(1-hydroxy-1-methyl-ethyl)-2-[2-[3-(4-piperidylmethyl)azetidin-1-yl]phenoxy]phenyl]-6-methyl-1H-pyrrolo[2,3-c]pyridin-7-one (72.0 mg, crude) as a yellow oil.

Step 8: Preparation of N-[3-(3-chloro-4-cyano-phenoxy)-2,2,4,4-tetramethyl-cyclobutyl]-2-[4-[[1-[2-[4-(1-hydroxy-1-methyl-ethyl)-2-(6-methyl-7-oxo-1H-pyrrolo[2,3-c]pyridin-4-yl)phenoxy]phenyl]azetidin-3-yl]methyl]-1-piperidyl]pyrimidine-5-carboxamide. To a solution of 4-[5-(1-hydroxy-1-methyl-ethyl)-2-[2-[3-(4-piperidylmethyl)azetidin-1-yl]phenoxy]phenyl]-6-methyl-1H-pyrrolo[2,3-c]pyridin-7-one (72.0 mg, 0.14 mmol, 1 equiv) and 2-chloro-N-[3-(3-chloro-4-cyano-phenoxy)-2,2,4,4-tetramethyl-cyclobutyl]pyrimidine-5-carboxamide (40.1 mg, 0.10 mmol, 0.7 equiv) in DMF (1 mL) was added DIEA (53.0 mg, 0.41 mmol, 0.07 mL, 3 equiv). The mixture was stirred at 50° C. for 1 h. The reaction mixture was filtered and concentrated in vacuo. The crude product was purified by prep-HPLC (column: Phenomenex Luna C18 150*25 mm*10 um; mobile phase: [water(FA)-ACN];gradient:60%-90% B over 10 min) to afford N-[3-(3-chloro-4-cyano-phenoxy)-2,2,4,4-tetramethyl-cyclobutyl]-2-[4-[[1-[2-[4-(1-hydroxy-1-methyl-ethyl)-2-(6-methyl-7-oxo-1H-pyrrolo[2,3-c]pyridin-4-yl)phenoxy]phenyl]azetidin-3-yl]methyl]-1-piperidyl]pyrimidine-5-carboxamide (34.3 mg, 0.04 mmol, 27% yield for 2 steps) as a white solid. ¹H NMR (400 MHz, MeOH-d₄) δ 8.73 (s, 2H), 7.72 (d, 1H, J=8.8 Hz), 7.65 (d, 1H, J=2.4 Hz), 7.43 (dd, TH, J=8.8, 2.4 Hz), 7.34 (d, 1H, J=2.8 Hz), 7.22 (s, 1H), 7.13 (d, 1H, J=2.4 Hz), 7.02-6.91 (m, 2H), 6.85-6.77 (m, 2H), 6.75-6.66 (m, 1H), 6.56-6.48 (m, 1H), 6.34 (d, 1H, J=2.8 Hz), 4.79 (s, 2H), 4.27 (s, 1H), 4.13 (s, 1H), 3.85 (t, 2H, J=8.0 Hz), 3.66 (s, 3H), 3.25 (t, 2H, J=6.8 Hz), 2.97-2.84 (m, 2H), 2.69-2.53 (m, 1H), 1.62 (d, 2H, J=14.0 Hz), 1.58 (s, 6H), 1.43 (d, 3H, J=6.0 Hz), 1.28 (s, 6H), 1.21 (s, 6H), 1.11-0.97 (m, 2H). LC-MS: MS (ES⁺): RT=2.643 min, m/z=909.5 [M+H⁺]; LCMS Method: 25.

### Example 64—Synthesis of N-[3-(3-chloro-4-cyano-phenoxy)-2,2,4,4-tetramethyl-cyclobutyl]-5-[4-[[1-[4-[4-(1-hydroxy-1-methyl-ethyl)-2-(6-methyl-7-oxo-1H-pyrrolo[2,3-c]pyridin-4-yl)phenoxy]-2,6-dimethyl-phenyl]azetidin-3-yl]methyl]-1-piperidyl]pyrazine-2-carboxamide (I-391)

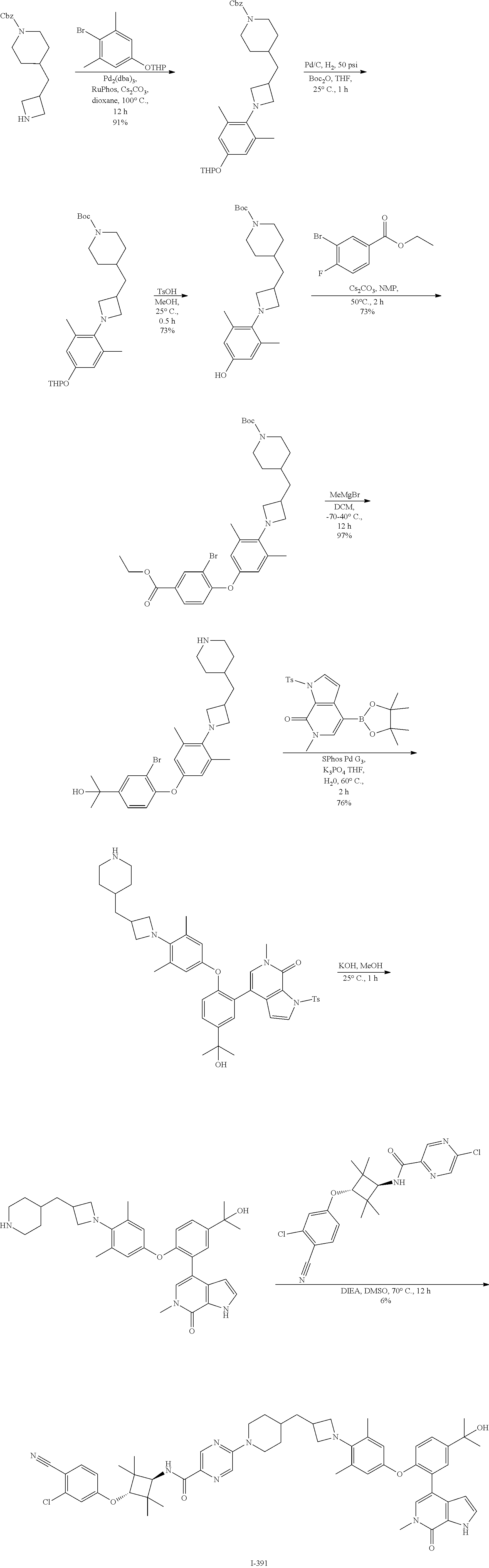

dimethyl-phenoxy)tetrahydropyran (702 mg, 2.46 mmol, 1.1 equiv) in dioxane (15 mL) was added CS₂CO₃ (2.19 g, 6.71 mmol, 3.0 equiv), benzyl 4-(azetidin-3-ylmethyl)piperidine-1-carboxylate (900 mg, 2.24 mmol, 1.0 equiv, TFA salt), RuPhos (104 mg, 224 μmol, 0.1 equiv) and Pd₂(dba)₃ (205 mg, 224 μmol, 0.1 equiv). The mixture was stirred at 100° C. for 12 h. The mixture was filtered and concentrated. The residue was purified by silica column chromatography on silica gel (Petroleum ether: Ethyl acetate from 50/1 to 5/1) to give benzyl 4-[[1-(2,6-dimethyl-4-tetrahydropyran-2-yloxy-phenyl)azetidin-3-yl]methyl]piperidine-1-carboxylate (1 g, 91% yield).

Step 2: Preparation of tert-butyl 4-[[1-(2,6-dimethyl-4-tetrahydropyran-2-yloxy-phenyl)azetidin-3-yl]methyl]piperidine-1-carboxylate. To a solution of benzyl 4-[[1-(2,6-dimethyl-4-tetrahydropyran-2-yloxy-phenyl)azetidin-3-yl]methyl]piperidine-1-carboxylate (1 g, 2.03 mmol, 1.0 equiv) in THF (15 mL) was added Pd/C (100 mg, 10% purity) and Boc₂O (1.33 g, 6.09 mmol. 1.4 mL, 3.0 equiv) under N₂. The mixture was stirred under H₂ (50 psi) at 25° C. for 1 h. The mixture was filtered and concentrated to give tert-butyl 4-[[1-(2,6-dimethyl-4-tetrahydropyran-2-yloxy-phenyl)azetidin-3-yl]methyl]piperidine-1-carboxylate (1 g, crude).

Step 3: Preparation of tert-butyl 4-[[1-(4-hydroxy-2,6-dimethyl-phenyl)azetidin-3-yl]methyl]piperidine-1-carboxylate. To a solution of tert-butyl 4-[[1-(2,6-dimethyl-4-tetrahydropyran-2-yloxy-phenyl)azetidin-3-yl]methyl]piperidine-1-carboxylate (1 g, 2.18 mmol, 1.0 equiv) in MeOH (10 mL) was added TsOH·H₂O (830 mg, 4.36 mmol, 2.0 equiv). The mixture was stirred at 25° C. for 0.5 h. The mixture was adjusted pH to 7 with DIEA and concentrated. The residue was purified by silica column chromatography on silica gel (Petroleum ether: Ethyl acetate from 50/1 to 2/1) to give tert-butyl 4-[[1-(4-hydroxy-2,6-dimethyl-phenyl)azetidin-3-yl]methyl]piperidine-1-carboxylate (600 mg, 73% yield).

Step 4: Preparation of tert-butyl 4-[[1-[4-(2-bromo-4-ethoxycarbonyl-phenoxy)-2,6-dimethyl-phenyl]azetidin-3-yl]methyl]piperidine-1-carboxylate. To a solution of tert-butyl 4-[[1-(4-hydroxy-2,6-dimethyl-phenyl)azetidin-3-yl]methyl]piperidine-1-carboxylate (600 mg, 1.60 mmol, 1.0 equiv) in NMP (8 mL) was added Cs₂CO₃ (1.04 g, 3.20 mmol, 2.0 equiv) and ethyl 3-bromo-4-fluorobenzoate (416 mg, 1.68 mmol, 1.1 equiv). The mixture was stirred at 50° C. for 2 h. The mixture was adjusted pH to 7 with AcOH at 0° C., diluted with water (15 mL) and extracted with ethyl acetate (20 mL*3). The combined organic layers were washed with brine (20 mL*3), dried over sodium sulfate, filtered and concentrated. The residue was purified by silica column chromatography on silica gel (Petroleum ether: Ethyl acetate from 50/1 to 3/1) to give tert-butyl 4-[[1-[4-(2-bromo-4-ethoxycarbonyl-phenoxy)-2,6-dimethyl-phenyl]azetidin-3-yl]methyl]piperidine-1-carboxylate (700 mg, 73% yield).

Step 5: Preparation of 2-[3-bromo-4-[3,5-dimethyl-4-[3-(4-piperidylmethyl)azetidin-1-yl]phenoxy]phenyl]propan-2-ol. To a solution of tert-butyl 4-[[1-[4-(2-bromo-4-ethoxycarbonyl-phenoxy)-2,6-dimethyl-phenyl]azetidin-3-yl]methyl]piperidine-1-carboxylate (700 mg, 1.16 mmol, 1.0 equiv) in DCM (7 mL) was added MeMgBr (3 M, 7.8 mL, 20.0 equiv) at −70° C. The mixture was stirred at 40° C. for 12 h. The mixture was quenched with saturated ammonium chloride solution (50 mL) at 0° C. and extracted with DCM (50 mL*3). The combined organic layers were washed with water (50 mL*3), dried over sodium sulfate, filtered and concentrated under reduced pressure to give 2-[3-bromo-4-[3,5-dimethyl-4-[3-(4-piperidylmethyl)azetidin-1-yl]phenoxy]phenyl]propan-2-ol (550 mg, 97% yield).

Step 6: Preparation of 4-[2-[3,5-dimethyl-4-[3-(4-piperidylmethyl)azetidin-1-yl]phenoxy]-5-(1-hydroxy-1-methyl-ethyl)phenyl]-6-methyl-1-(p-tolylsulfonyl)pyrrolo[2,3-c]pyridin-7-one. To a solution of 6-methyl-1-(p-tolylsulfonyl)-4-(4,4,5,5-tetramethyl-1,3,2-dioxaborolan-2-yl)pyrrolo[2,3-c]pyridin-7-one (395 mg, 923 mol, 1.0 equiv) in THF (10 mL) and H₂O (2 mL) was added SPhos Pd G3 (72 mg, 92 μmol, 0.1 equiv), 2-[3-bromo-4-[3,5-dimethyl-4-[3-(4-piperidylmethyl)azetidin-1-yl]phenoxy]phenyl]propan-2-ol (450 mg, 923 mol, 1.0 equiv) and K₃PO₄ (588 mg, 2.77 mmol, 3.0 equiv). The mixture was stirred at 60° C. for 2 h. The mixture was diluted with water (15 mL) and extracted with ethyl acetate (20 mL*3). The combined organic layers were washed with brine (20 mL*3), dried over sodium sulfate, filtered and concentrated. The residue was purified by prep-HPLC (column: Phenomenex luna C18 150*25 mm*10 um; mobile phase: [water (FA)-ACN]; gradient: 20%-50% B over 10 min) to give 4-[2-[3,5-dimethyl-4-[3-(4-piperidylmethyl)azetidin-1-yl]phenoxy]-5-(1-hydroxy-1-methyl-ethyl) phenyl]-6-methyl-1-(p-tolylsulfonyl)pyrrolo[2,3-c]pyridin-7-one (500 mg, 76% yield).

Step 7: Preparation of 4-[2-[3,5-dimethyl-4-[3-(4-piperidylmethyl)azetidin-1-yl]phenoxy]-5-(1-hydroxy-1-methyl-ethyl)phenyl]-6-methyl-1H-pyrrolo[2,3-c]pyridin-7-one. To a solution of 4-[2-[3,5-dimethyl-4-[3-(4-piperidylmethyl)azetidin-1-yl]phenoxy]-5-(1-hydroxy-1-methyl-ethyl)phenyl]-6-methyl-1-(p-tolylsulfonyl)pyrrolo[2,3-c]pyridin-7-one (200 mg, 282 μmol, 1.0 equiv) in MeOH (2 mL) was added KOH (1 M, 2.8 mL, 10 equiv). The mixture was stirred at 25° C. for 1 h. The mixture was adjusted pH to 7 with AcOH, diluted with water (15 mL) and extracted with DCM (20 mL*3). The combined organic layers were washed with water (20 mL*3), dried over sodium sulfate, filtered and concentrated to give 4-[2-[3,5-dimethyl-4-[3-(4-piperidylmethyl)azetidin-1-yl]phenoxy]-5-(1-hydroxy-1-methyl-ethyl)phenyl]-6-methyl-1H-pyrrolo[2,3-c]pyridin-7-one (150 mg, crude).

Step 8: Preparation of N-[3-(3-chloro-4-cyano-phenoxy)-2,2,4,4-tetramethyl-cyclobutyl]-5-[4-[[1-[4-[4-(1-hydroxy-1-methyl-ethyl)-2-(6-methyl-7-oxo-1H-pyrrolo[2,3-c]pyridin-4-yl)phenoxy]-2,6-dimethyl-phenyl]azetidin-3-yl]methyl]-1-piperidyl]pyrazine-2-carboxamide. To a solution of 5-chloro-N-[3-(3-chloro-4-cyano-phenoxy)-2,2,4,4-tetramethyl-cyclobutyl]pyrazine-2-carboxamide (113 mg, 270 mol, 1.0 equiv) in DMSO (2 mL) was added DIEA (175 mg, 1.35 mmol, 5.0 equiv) and 4-[2-[3,5-dimethyl-4-[3-(4-piperidylmethyl)azetidin-1-yl]phenoxy]-5-(1-hydroxy-1-methyl-ethyl) phenyl]-6-methyl-1H-pyrrolo[2,3-c]pyridin-7-one (150 mg, 270 umol, 1.0 equiv). The mixture was stirred at 70° C. for 12 h. The mixture was concentrated and purified by prep-HPLC (column: Waters xbridge 150*25 mm 10 um; mobile phase: [water (NH₄HCO₃)-ACN]; gradient: 76%-96% B over 8 min) and prep-HPLC (column: Phenomenex luna Cis 150*25 mm*10 um; mobile phase: [water (FA)-ACN]; gradient: 60%-90% B over 10 min) to give N-[3-(3-chloro-4-cyano-phenoxy)-2,2,4,4-tetramethyl-cyclobutyl]-5-[4-[[1-[4-[4-(1-hydroxy-1-methyl-ethyl)-2-(6-methyl-7-oxo-1H-pyrrolo[2,3-c]pyridin-4-yl)phenoxy]-2,6-dimethyl-phenyl]azetidin-3-yl]methyl]-1-piperidyl]pyrazine-2-carboxamide (15 mg, 6% yield).¹H NMR (400 MHz, CDCl₃): δ 9.92 (s, 1H), 8.82 (d, J=1.2 Hz, 1H), 7.99 (s, 1H), 7.76 (d, J=8.8 Hz, 1H), 7.62 (d, J=2.4 Hz, 1H), 7.57 (d, J=8.8 Hz, 1H), 7.40 (m, J=2.4, 8.8 Hz, 1H), 7.24 (t, J=2.8 Hz, 1H), 7.10 (s, 1H), 6.98 (d, J=2.4 Hz, 1H), 6.92 (d, J=8.8 Hz, 1H), 6.82 (m, J=2.4, 8.8 Hz, 1H), 6.48 (s, 2H), 6.41 (t, J=2.4 Hz, 1H), 4.47 (d, J=13.2 Hz, 2H), 4.23 (t, J=7.2 Hz, 2H), 4.14 (d, J=8.8 Hz, 1H), 4.07 (s, 1H), 3.70 (t, J=6.4 Hz, 2H), 3.65 (s, 3H), 3.03-2.91 (m, 2H), 2.75-2.64 (m, 1H), 2.24 (s, 6H), 1.84-1.78 (m, 3H), 1.64 (s, 7H), 1.62 (s, 2H), 1.27 (s, 8H), 1.22 (s, 6H). LC-MS: MS (ES⁺). RT=2.317 min, m/z=937.7 [M+H⁺]; LCMS Method: 25.

### Example 65—Synthesis of N-[3-(3-chloro-4-cyano-phenoxy)-2,2,4,4-tetramethyl-cyclobutyl]-5-[4-[[1-[4-[4-(1-hydroxy-1-methyl-ethyl)-2-(6-methyl-7-oxo-1H-pyrrolo[2,3-c]pyridin-4-yl)phenoxy]-3-methoxy-phenyl]azetidin-3-yl]methyl]-1-piperidyl]pyrazine-2-carboxamide (I-392)

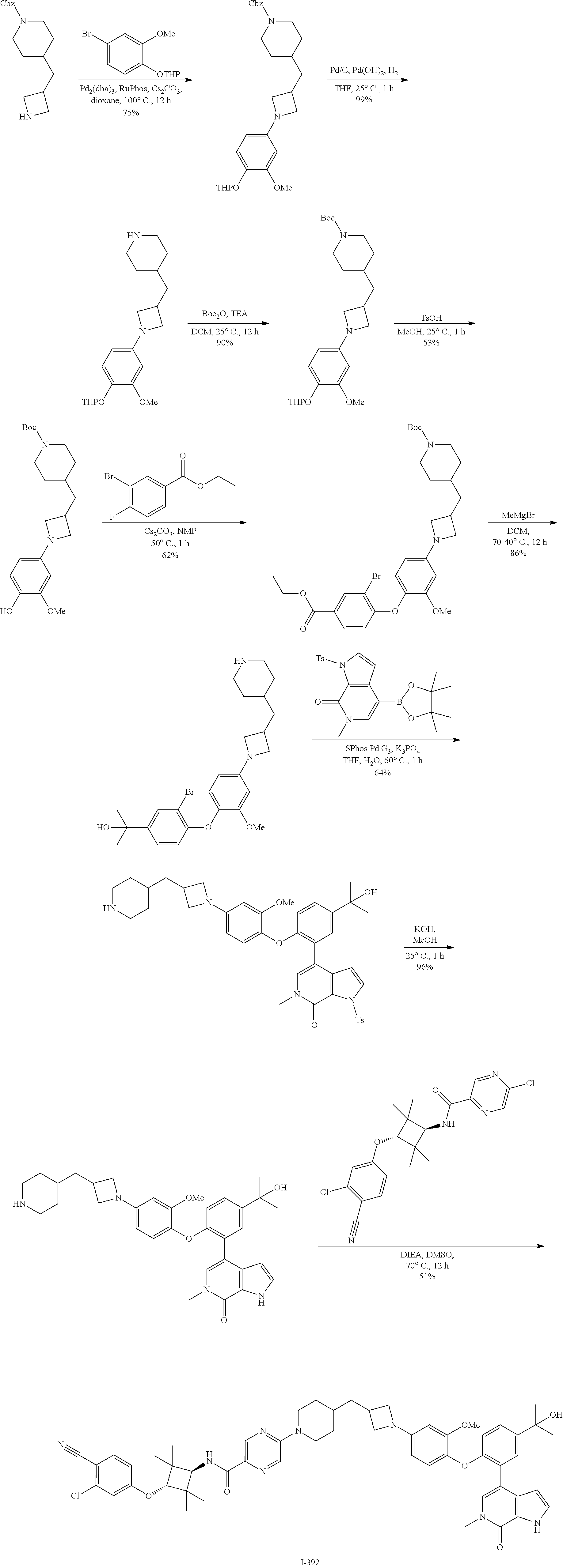

Step 1: Preparation of benzyl 4-[[1-(3-methoxy-4-tetrahydropyran-2-yloxy-phenyl)azetidin-3-yl]methyl]piperidine-1-carboxylate. To a solution of benzyl 4-(azetidin-3-ylmethyl)piperidine-1-carboxylate (668 mg, 2.32 mmol, 1.0 equiv) in dioxane (7 mL) was added Cs₂CO₃ (2.26 g, 6.95 mmol, 3.0 equiv), 2-(4-bromo-2-methoxy-phenoxy)tetrahydropyran (798 mg, 2.78 mmol, 1.2 equiv), RuPhos (108 mg, 232 μmol, 0.1 equiv) andPd₂(dba)₃ (212 mg, 232 μmol, 0.1 equiv). The mixture was stirred at 100° C. for 12 h. The reaction mixture was filtered and concentrated under reduced pressure to give a residue. The residue was purified by silica column chromatography on silica gel (Petroleum ether/Ethyl acetate=I/O to 3/1) to give benzyl 4-[[1-(3-methoxy-4-tetrahydropyran-2-yloxy-phenyl)azetidin-3-yl]methyl]piperidine-1-carboxylate (0.86 g, 75% yield) as a yellow oil.

Step 2: Preparation of 4-[[1-(3-methoxy-4-tetrahydropyran-2-yloxy-phenyl)azetidin-3-yl]methyl]piperidine. To a solution of benzyl 4-[[1-(3-methoxy-4-tetrahy dropyran-2-yloxy-phenyl)azetidin-3-yl]methyl]piperidine-1-carboxylate (860 mg, 1.74 mmol, 1.0 equiv) in THF (10 mL) was added Pd/C (0.1 g, 10% purity) and Pd(OH)₂ (0.1 g, 10% purity) under N₂. The mixture was stirred under H₂ (50 psi) at 25° C. for 1 h. The reaction mixture was filtered and concentrated under reduced pressure to give 4-[[1-(3-methoxy-4-tetrahydropyran-2-yloxy-phenyl)azetidin-3-yl]methyl]piperidine (626 mg, 99% yield).

Step 3: Preparation of tert-butyl 4-[[1-(3-methoxy-4-tetrahydropyran-2-yloxy-phenyl)azetidin-3-yl]methyl]piperidine-1-carboxylate. To a solution of 4-[[1-(3-methoxy-4-tetrahydropyran-2-yloxy-phenyl)azetidin-3-yl]methyl]piperidine (626 mg, 1.74 mmol, 1.0 equiv) in DCM (10 mL) was added Boc₂₀ (569 mg, 2.60 mmol, 0.6 mL, 1.5 equiv) and TEA (0.7 mL). The mixture was stirred at 25° C. for 12 h. The mixture was concentrated and purified by column chromatography (SiO₂, Petroleum ether/Ethyl acetate=I/O to 3/1) to give tert-butyl 4-[[1-(3-methoxy-4-tetrahydropyran-2-yloxy-phenyl)azetidin-3-yl]methyl]piperidine-1-carboxylate (720 mg, 90% yield).

Step 4: Preparation of tert-butyl 4-[[1-(4-hydroxy-3-methoxy-phenyl)azetidin-3-yl]methyl]piperidine-1-carboxylate. To a solution of tert-butyl 4-[[1-(3-methoxy-4-tetrahydropyran-2-yloxy-phenyl)azetidin-3-yl]methyl]piperidine-1-carboxylate (720 mg, 1.56 mmol, 1.0 equiv) in MeOH (14 mL) was added TsOH·H₂O (595 mg, 3.13 mmol, 2.0 equiv). The mixture was stirred at 25° C. for 1 h. The mixture was adjusted pH to 7 with DIEA and concentrated to give a residue. The residue was purified by column chromatography (SiO₂, Petroleum ether/Ethyl acetate=I/O to 2/1) to give tert-butyl 4-[[1-(4-hydroxy-3-methoxy-phenyl)azetidin-3-yl]methyl]piperidine-1-carboxylate (310 mg, 53% yield).

Step 5: Preparation of tert-butyl 4-[[1-[4-(2-bromo-4-ethoxycarbonyl-phenoxy)-3-methoxy-phenyl]azetidin-3-yl]methyl]piperidine-1-carboxylate. To a solution of tert-butyl 4-[[1-(4-hydroxy-3-methoxy-phenyl)azetidin-3-yl]methyl]piperidine-1-carboxylate (310 mg, 823 μmol, 1.0 equiv) in NMP (4 mL) was added Cs₂CO₃ (537 mg, 1.65 mmol, 2.0 equiv) and ethyl 3-bromo-4-fluoro-benzoate (214 mg, 865 μmol, 1.1 equiv). The mixture was stirred at 50° C. for 1 h. The mixture was adjusted pH to 7 with AcOH at 0° C., diluted with water (15 mL) and extracted with ethyl acetate (20 mL*3). The combined organic layers were washed with brine (20 mL*3), dried over sodium sulfate, filtered and concentrated. The residue was purified by silica column chromatography on silica gel (Petroleum ether: Ethyl acetate from 50/1 to 3/1) to give tert-butyl 4-[[1-[4-(2-bromo-4-ethoxy carbonyl-phenoxy)-3-methoxy-phenyl]azetidin-3-yl]methyl]piperidine-1-carboxylate (310 mg, 62% yield).

Step 6: Preparation of 2-[3-bromo-4-[2-methoxy-4-[3-(4-piperidylmethyl) azetidin-1-yl]phenoxy]phenyl]propan-2-ol. To a solution of tert-butyl 4-[[1-[4-(2-bromo-4-ethoxycarbonyl-phenoxy)-3-methoxy-phenyl]azetidin-3-yl]methyl]piperidine-1-carboxylate (300 mg, 497 mol, 1.0 equiv) in DCM (3 mL) was added MeMgBr (3 M, 3.3 mL, 20.0 equiv) at −70° C. The mixture was stirred at 40° C. for 12 h. The mixture was quenched with saturated ammonium chloride solution (20 mL) at 0° C. and extracted with DCM (20 mL*3). The combined organic layers were washed with water (20 mL*3), dried over sodium sulfate, filtered and concentrated to give 2-[3-bromo-4-[2-methoxy-4-[3-(4-piperidylmethyl)azetidin-1-yl]phenoxy]phenyl]propan-2-ol (210 mg, 86% yield).

Step 7: Preparation of 4-[5-(1-hydroxy-1-methyl-ethyl)-2-[2-methoxy-4-[3-(4-piperidylmethyl)azetidin-1-yl]phenoxy]phenyl]-6-methyl-1-(p-tolylsulfonyl)pyrrolo[2,3-c]pyridin-7-one. To a solution of 2-[3-bromo-4-[2-methoxy-4-[3-(4-piperidylmethyl) azetidin-1-yl]phenoxy]phenyl]propan-2-ol (150 mg, 306 mol, 1.0 equiv) in THF (3 mL) and H₂O (0.6 mL) was added SPhos Pd G3 (24 mg, 31 mol, 0.1 equiv), 6-methyl-1-(p-tolylsulfonyl)-4-(4,4,5,5-tetramethyl-1,3,2-dioxaborolan-2-yl)pyrrolo[2,3-c]pyridin-7-one (144 mg, 337 mol, 1.1 equiv) and K₃PO₄ (195 mg, 919 μmol, 3.0 equiv). The mixture was stirred at 60° C. for 1 h. The mixture was diluted with water (10 mL) and extracted with ethyl acetate (15 mL*3). The combined organic layers were washed with brine (20 mL*3), dried over sodium sulfate, filtered and concentrated. The residue was purified by prep-HPLC (column: Phenomenex luna C18 150*25 mm*10 um; mobile phase: [water (FA)-ACN]; gradient: 22%-52% B over 10 min) to give 4-[5-(1-hydroxy-1-methyl-ethyl)-2-[2-methoxy-4-[3-(4-piperidylmethyl)azetidin-1-yl]phenoxy]phenyl]-6-methyl-1-(p-tolylsulfonyl)pyrrolo [2,3-c]pyridin-7-one (140 mg, 64% yield).

Step 8: Preparation of 4-[5-(1-hydroxy-1-methyl-ethyl)-2-[2-methoxy-4-[3-(4-piperidylmethyl)azetidin-1-yl]phenoxy]phenyl]-6-methyl-1H-pyrrolo[2,3-c]pyridin-7-one. To a solution of 4-[5-(1-hydroxy-1-methyl-ethyl)-2-[2-methoxy-4-[3-(4-piperidylmethyl)azetidin-1-yl]phenoxy]phenyl]-6-methyl-1-(p-tolylsulfonyl)pyrrolo[2,3-c]pyridin-7-one (80 mg, 113 mol, 1.0 equiv) in MeOH (1 mL) was added KOH (1 M, 1.1 mL, 10.0 equiv). The mixture was stirred at 25° C. for 1 h. The mixture was adjusted pH to 7 with AcOH at 0° C. Water (30 mL) was added to the mixture. The mixture was lyophilized to give 4-[5-(1-hydroxy-1-methyl-ethyl)-2-[2-methoxy-4-[3-(4-piperidylmethyl)azetidin-1-yl]phenoxy]phenyl]-6-methyl-1H-pyrrolo[2,3-c]pyridin-7-one (60 mg, 96% yield).

Step 9: Preparation of N-[3-(3-chloro-4-cyano-phenoxy)-2,2,4,4-tetramethyl-cyclobutyl]-5-[4-[[1-[4-[4-(1-hydroxy-1-methyl-ethyl)-2-(6-methyl-7-oxo-1H-pyrrolo[2,3-c]pyridin-4-yl)phenoxy]-3-methoxy-phenyl]azetidin-3-yl]methyl]-1-piperidyl]pyrazine-2-carboxamide. To a solution of 4-[5-(1-hydroxy-1-methyl-ethyl)-2-[2-methoxy-4-[3-(4-piperidylmethyl)azetidin-1-yl]phenoxy]phenyl]-6-methyl-¹H-pyrrolo[2,3-c]pyridin-7-one (60 mg, 108 mol, 1.0 equiv) in DMSO (1 mL) was added DIEA (70 mg, 539 mol, 5.0 equiv) and 5-chloro-N-[3-(3-chloro-4-cyano-phenoxy)-2,2,4,4-tetramethyl-cyclobutyl]pyrazine-2-carboxamide (41 mg, 97 μmol, 0.9 equiv). The mixture was stirred at 70° C. for 12 h. The mixture was concentrated and purified by prep-HPLC (column: Phenomenex luna C₁₈ 150*25 mm*10 um; mobile phase: [water (FA)-ACN]; gradient: 80%-100% B over 10 min) to give N-[3-(3-chloro-4-cyano-phenoxy)-2,2,4,4-tetramethyl-cyclobutyl]-5-[4-[[1-[4-[4-(1-hydroxy-1-methyl-ethyl)-2-(6-methyl-7-oxo-1H-pyrrolo[2,3-c]pyridin-4-yl)phenoxy]-3-methoxy-phenyl]azetidin-3-yl]methyl]-1-piperidyl]pyrazine-2-carboxamide (52 mg, 51% yield). ¹H NMR (400 MHz, CDCl₃): δ 10.07 (s, 1H), 8.77 (s, 1H), 7.94 (s, 1H), 7.71 (d, J=8.4 Hz, 1H), 7.56-7.48 (m, 2H), 7.23 (d, J=9.2 Hz, 3H), 7.17 (s, 1H), 6.92 (s, 1H), 6.77 (d, J=8.8 Hz, 2H), 6.63 (d, J=7.6 Hz, 1H), 6.46 (s, 1H), 5.99 (s, 1H), 5.90 (d, J=8.4 Hz, 1H), 4.49-4.38 (m, 2H), 4.09 (d, J=8.8 Hz, 1H), 4.03-3.95 (m, 3H), 3.70 (s, 3H), 3.63 (s, 3H), 3.42 (t, J=6.0 Hz, 2H), 2.92 (t, J=12.8 Hz, 2H), 2.84-2.75 (m, 1H), 1.75 (s, 2H), 1.61 (d, J=5.6 Hz, 3H), 1.56 (s, 6H), 1.22 (s, 8H), 1.16 (s, 6H). LC-MS: MS (ES⁺): RT=2.800 min, m/z=939.7 [M+H⁺]; LCMS Method: 25.

### Example 66—Synthesis of Additional Compounds

The following additional compounds were prepared based on procedures described herein: I-104, I-105, I-106, I-108, I-109, I-110, I-111, I-113, I-114, 1-115, I-116, I-117, I-118, I-119, I-120, I-121, I-123, I-124, I-126, 1-127, I-128, I-129, I-130, I-131, 1-132, I-133, I-135, I-136, I-137, I-138, I-139, I-140, I-141, 1-142, I-143, I-144, I-145, I-146, 1-147, I-148, I-149, I-150, I-151, I-152, I-153, I-154, I-155, 1-156, I-157, I-158, I-159, I-160, 1-161, I-162, I-164, I-165, I-166, I-167, I-168, I-169, I-170, 1-171, I-172, I-173, I-174, I-175, 1-176, I-177, I-178, 1-179, 1-180, I-181, I-182,1-183,1-184,1-185,1-187, 1-188,1-190, 1-191, 1-192,1-193, 1-194, I-195, 1-196, 1-197,1-198,1-199,1-200,1-201,1-202, 1-203,1-204, 1-205, 1-206,1-207, 1-208, I-209, I-210, I-211, I-212, I-213, I-214, 1-215, I-216, I-217, I-218, I-219, I-220, I-221, I-222, I-224, I-225, I-226, I-227, I-228, I-229, 1-230, I-232, I-233, I-234, I-235, 1-236, 1-238, 1-239, 1-240, 1-241, 1-242, 1-244, 1-245, 1-246, 1-247, 1-248, 1-250, 1-251, 1-252, 1-255, 1-256, 1-257, I-258, I-259, I-260, I-261, I-262, I-263, 1-264, I-265, I-267, I-268, I-269, 1-271, I-272, I-273, I-275, 1-276, 1-277, 1-278, 1-279, 1-280, 1-283, 1-284, 1-285, 1-286, 1-289, 1-290, 1-291, I-293, I-294, I-296, I-299, I-300, I-301, I-302, 1-303, I-304, I-305, I-306, I-307, I-308, I-310, I-311, 1-312, 1-313, 1-314,1-315,1-316,1-317,1-318,1-319, 1-320,1-321, 1-322, 1-323,1-324, 1-325, I-326, I-327, I-328, I-330, I-331, I-332, 1-334, I-335, I-336, I-337, I-338, I-339, I-340, I-341, 1-342, 1-343, 1-346,1-347,1-348,1-349,1-350,1-351, 1-352,1-353, 1-354, 1-355,1-358, 1-359, 1-360, 1-361, I-362, I-363, I-364, I-365, 1-366, I-367, I-368, I-369, I-370, I-371, I-372, I-373, I-374, I-375, I-376, I-378, I-379, I-380, 1-381, I-382, I-383, I-384, I-385, 1-386,1-388, 1-389, 1-390, 1-393, and I-394.

Exemplary compounds above were analyzed by LC-MS. Results are provided below.

TABLE 4

| Compound No. | Observed Mass | LCMS Method |
|---|---|---|
| I-104 | 480.2, 959.3 | 25 |
| I-105 | 448.8, 896.3 | 25 |
| I-106 | 467.1, 933.2 | 25 |
| I-108 | 476.1, 951.2 | 25 |
| I-109 | 932.6 | 05 |
| I-110 | 883.4 | 25 |
| I-111 | 475.7, 950.2 | 25 |
| I-113 | 481.7, 962.2 | 25 |
| I-114 | 944.2 | 25 |
| I-115 | 423.1, 845.2 | 25 |
| I-116 | 950.6 | 40 |
| I-117 | 844.5 | 40 |
| I-118 | 480.2, 959.2 | 25 |
| I-119 | 472.8, 944.2 | 25 |
| I-120 | 945.2 | 25 |
| I-121 | 963.2 | 25 |
| I-123 | 898.3 | 25 |
| I-124 | 965.2 | 25 |
| I-126 | 945.3 | 25 |
| I-127 | 967.2 | 25 |
| I-128 | 967.2 | 25 |
| I-129 | 486.7, 972.3 | 25 |
| I-130 | 945.6 | 80 |
| I-131 | 963.7 | 80 |
| I-132 | 887.6 | 05 |
| I-133 | 466.1, 931.2 | 25 |
| I-135 | 481.6, 962.3 | 25 |
| I-136 | 482.1, 963.1 | 25 |
| I-137 | 428.7, 856.3 | 25 |
| I-138 | 460.1, 919.2 | 25 |
| I-139 | 475.1, 949.2 | 25 |
| I-140 | 475.6, 950.2 | 25 |
| I-141 | 868.7 | 05 |
| I-142 | 902.6 | 15 |
| I-143 | 451.3, 901.2 | 25 |
| I-144 | 475.2, 949.2 | 25 |
| I-145 | 473.1, 945.2 | 25 |
| I-146 | 413.7, 826.3 | 25 |
| I-147 | 451.7, 902.2 | 25 |
| I-148 | 443.7, 886.3 | 25 |
| I-149 | 422.7, 844.2 | 25 |
| I-150 | 484.2, 967.2 | 25 |
| I-151 | 467.3, 933.2 | 25 |
| I-152 | 474.3, 947.3 | 25 |
| I-153 | 511.2, 1021.2 | 25 |
| I-154 | 483.2, 965.2 | 25 |
| I-155 | 483.3, 965.2 | 25 |
| I-156 | 884.8 | 80 |
| I-157 | 472.1, 943.2 | 25 |
| I-158 | 465.7, 930.2 | 25 |
| I-159 | 466.6, 932.2 | 25 |
| I-160 | 475.7, 950.3 | 25 |
| I-161 | 484.1, 967.2 | 25 |
| I-162 | 484.2, 967.1 | 25 |
| I-164 | 469.2, 937.2 | 25 |
| I-165 | 468.2, 935.2 | 25 |
| I-166 | 483.2, 965.2 | 25 |
| I-167 | 483.6, 966.2 | 25 |
| I-168 | 482.8, 964.2 | 25 |
| I-169 | 476.7, 952.2 | 25 |
| I-170 | 494.7, 988.3 | 25 |
| I-171 | 495.8, 990.2 | 25 |
| I-172 | 486.8, 972.2 | 25 |
| I-173 | 481.2, 961.3 | 25 |
| I-174 | 481.6, 962.2 | 25 |
| I-175 | 489.6, 978.2 | 25 |
| I-176 | 475.6, 950.2 | 25 |
| I-177 | 463.7, 926.3 | 25 |
| I-178 | 496.2, 991.3 | 25 |
| I-179 | 444.2, 887.3 | 25 |
| I-180 | 944.3 | 25 |
| I-181 | 480.7, 959.2 | 25 |
| I-182 | 472.7, 944.3 | 25 |
| I-183 | 511.2, 1021.3 | 25 |
| I-184 | 482.6, 964.2 | 25 |
| I-185 | 476.2, 951.2 | 25 |
| I-187 | 478.2, 955.3 | 25 |
| I-188 | 483.7, 966.2 | 25 |
| I-190 | 465.2, 929.3 | 25 |
| I-191 | 475.0, 949.2 | 25 |
| I-192 | 912.6 | 40 |
| I-193 | 884.5 | 25 |
| I-194 | 884.5 | 25 |
| I-195 | 463.8, 926.3 | 25 |
| I-196 | 464.2, 927.3 | 25 |
| I-197 | 435.1, 869.3 | 25 |
| I-198 | 407.7, 814.2 | 25 |
| I-199 | 415.7, 830.3 | 25 |
| I-200 | 422.7, 844.3 | 25 |
| I-201 | 427.7, 854.3 | 25 |
| I-202 | 435.7, 870.3 | 25 |
| I-203 | 434.6, 868.3 | 25 |
| I-204 | 443.3, 885.3 | 25 |
| I-205 | 481.6, 962.2 | 25 |
| I-206 | 481.2, 961.3 | 25 |
| I-207 | 469.1, 937.2 | 25 |
| I-208 | 462.7, 924.3 | 25 |
| I-209 | 480.2, 959.3 | 25 |
| I-210 | 457.7, 914.2 | 25 |
| I-211 | 414.7, 828.2 | 25 |
| I-212 | 883.2 | 25 |
| I-213 | 452.6, 904.3 | 25 |
| I-214 | 500.3, 999.5 | 25 |
| I-215 | 481.2, 961.2 | 25 |
| I-216 | 901.2 | 25 |
| I-217 | 475.2, 949.3 | 25 |
| I-218 | 842.2 | 25 |
| I-219 | 456.2, 911.4 | 25 |
| I-220 | 458.7, 916.2 | 25 |
| I-221 | 970.4 | 25 |
| I-222 | 915.2 | 25 |
| I-224 | 959.4 | 25 |
| I-225 | 932.2 | 25 |
| I-226 | 972.4 | 25 |
| I-227 | 915.2 | 25 |
| I-228 | 922.4 | 25 |
| I-229 | 915.2 | 25 |
| I-230 | 471.7, 942.3 | 25 |
| I-232 | 909.4 | 25 |
| I-233 | 882.4 | 25 |
| I-234 | 924.2 | 25 |
| I-235 | 923.4 | 25 |
| I-236 | 910.2 | 25 |
| I-238 | 896.2 | 25 |
| I-239 | 933.4 | 25 |
| I-240 | 968.4 | 25 |
| I-241 | 940.4 | 25 |
| I-242 | 910.2 | 25 |
| I-244 | 876.4 | 25 |
| I-245 | 934.4 | 25 |
| I-246 | 911.2 | 25 |
| I-247 | 931.4 | 25 |
| I-248 | 916.4 | 25 |
| I-250 | 920.4 | 25 |
| I-251 | 890.4 | 25 |
| I-252 | 875.4 | 25 |
| I-255 | 939.4 | 25 |
| I-256 | 925.4 | 25 |
| I-257 | 946.6 | 25 |
| I-258 | 954.4 | 25 |
| I-259 | 881.4 | 25 |
| I-260 | 897.4 | 25 |
| I-261 | 932.4 | 25 |
| I-262 | 945.4 | 25 |
| I-263 | 944.6 | 25 |
| I-264 | 963.2 | 25 |
| I-265 | 876.4 | 25 |
| I-267 | 917.4 | 25 |
| I-268 | 895.4 | 25 |
| I-269 | 925.4 | 25 |
| I-271 | 919.4 | 25 |
| I-272 | 947.4 | 25 |
| I-273 | 954.4 | 25 |
| I-275 | 947.4 | 25 |
| I-276 | 920.4 | 25 |
| I-277 | 919.4 | 25 |
| I-278 | 906.4 | 25 |
| I-279 | 894.4 | 25 |
| I-280 | 881.4 | 25 |
| I-283 | 948.4 | 25 |
| I-284 | 920.4 | 25 |
| I-285 | 934.4 | 25 |
| I-286 | 866.4 | 25 |
| I-289 | 917.4 | 25 |
| I-290 | 848.4 | 25 |
| I-291 | 929.4 | 25 |
| I-293 | 974.4 | 25 |
| I-294 | 956.4 | 25 |
| I-296 | 888.4 | 25 |
| I-299 | 895.4 | 25 |
| I-300 | 897.4 | 25 |
| I-301 | 868.4 | 25 |
| I-302 | 869.4 | 25 |
| I-303 | 883.4 | 25 |
| I-304 | 895.4 | 25 |
| I-305 | 870.4 | 25 |
| I-306 | 882.6 | 40 |
| I-307 | 884.4 | 25 |
| I-308 | 923.4 | 25 |
| I-310 | 846.4 | 25 |
| I-311 | 895.4 | 25 |
| I-312 | 925.4 | 25 |
| I-313 | 885.4 | 25 |
| I-314 | 924.4 | 25 |
| I-315 | 885.4 | 25 |
| I-316 | 880.4 | 25 |
| I-317 | 881.4 | 25 |
| I-318 | 922.7 | 40 |
| I-319 | 881.4 | 25 |
| I-320 | 868.4 | 25 |
| I-321 | 866.4 | 25 |
| I-322 | 880.5 | 25 |
| I-323 | 881.4 | 25 |
| I-324 | 894.4 | 25 |
| I-325 | 937.4 | 25 |
| I-326 | 936.4 | 25 |
| I-327 | 867.4 | 25 |
| I-328 | 939.4 | 25 |
| I-330 | 853.4 | 25 |
| I-331 | 907.4 | 25 |
| I-332 | 909.4 | 25 |
| I-334 | 910.4 | 25 |
| I-335 | 868.4 | 25 |
| I-336 | 879.4 | 25 |
| I-337 | 959.4 | 25 |
| I-338 | 908.4 | 25 |
| I-339 | 870.4 | 25 |
| I-340 | 871.4 | 25 |
| I-341 | 869.4 | 25 |
| I-342 | 881.5 | 25 |
| I-343 | 881.5 | 25 |
| I-346 | 910.4 | 25 |
| I-347 | 908.4 | 40 |
| I-348 | 908.4 | 25 |
| I-349 | 908.4 | 25 |
| I-350 | 909.4 | 25 |
| I-351 | 907.4 | 25 |
| I-352 | 881.4 | 25 |
| I-353 | 881.4 | 25 |
| I-354 | 908.4 | 40 |
| I-355 | 854.4 | 25 |
| I-358 | 439.9 | 05 |
| I-359 | 426.3 | 25 |
| I-360 | 837.6 | 25 |
| I-361 | 851.4 | 25 |
| I-362 | 923.4 | 25 |
| I-363 | 823.5 | 25 |
| I-364 | 850.4 | 25 |
| I-365 | 837.4 | 25 |
| I-366 | 936.4 | 25 |
| I-367 | 935.4 | 40 |
| I-368 | 853.4 | 25 |
| I-369 | 853.4 | 25 |
| I-370 | 854.4 | 25 |
| I-371 | 796.4 | 25 |
| I-372 | 907.4 | 25 |
| I-373 | 943.4 | 25 |
| I-374 | 854.4 | 25 |
| I-375 | 894.4 | 40 |
| I-376 | 909.4 | 25 |
| I-378 | 950.6 | 40 |
| I-379 | 893.4 | 40 |
| I-380 | 894.6 | 40 |
| I-381 | 895.4 | 25 |
| I-382 | 894.4 | 25 |
| I-383 | 893.4 | 25 |
| I-384 | 905.6 | 25 |
| I-385 | 891.6 | 25 |
| I-386 | 836.4 | 01 |
| I-388 | 894.4 | 25 |
| I-389 | 929.4 | 25 |
| I-390 | 879.4 | 25 |
| I-391 | 937.7 | 25 |
| I-394 | 939.6 | 25 |

### Example 67—Assay for Binding Affinity to Androgen Receptor

Exemplary compounds were tested for ability to bind to the androgen receptor. Experimental procedures and results are provided below.

### Part I—Experimental Procedure

Fractions of cell cytosol (106 cell/point) were incubated for 24 hr at 4° C. with 1 nM [³H]methyltrienolone in the absence or presence of the test compound in a buffer containing 25 mM Hepes-Tris (pH 7.4), 1 mM EDTA, 10 mM Na₂MoO₄, 2 mM DTT, 5 μM triamcinolone acetonide, and 10% glycerol. Nonspecific binding was determined in the presence of 1 M testosterone. Following incubation, the samples were filtered rapidly under vacuum through glass fiber filters (GF/B, Packard) presoaked with 0.3% PEI and rinsed several times with ice-cold 50 mM Tris-HCl using a 96-sample cell harvester (Unifilter, Packard). The filters were dried then counted for radioactivity in a scintillation counter (Topcount, Packard) using a scintillation cocktail (Microscint 0, Packard). The results are expressed as a percent inhibition of the control radioligand specific binding. The standard reference compound is testosterone, which is tested in each experiment at several concentrations to obtain a competition curve from which its IC₅₀ is calculated.

### Part II—Results

Results showing ability of exemplary compounds to bind to the androgen receptor are provided in Table 5 below. The symbol “++++” indicates a Ki less than 0.05 μM. The symbol “+++” indicates an Ki in the range of 0.05 M to 0.5 μM. The symbol “++” indicates a Ki in the range of greater than 0.5 μM to 2.5 μM. The symbol “+” indicates a Ki greater than 2.5 μM. The symbol “N/A” indicates that no data was available.

TABLE 5

| Compound No. | Ki | Compound No. | Ki |
|---|---|---|---|
| I-1 | ++++ | I-93 | ++++ |
| I-3 | ++++ | I-100 | ++++ |
| I-8 | ++++ | I-101 | ++++ |
| I-11 | ++++ | I-99 | ++++ |
| I-13 | ++++ | I-118 | ++++ |
| I-18 | ++++ | I-119 | ++++ |
| I-39 | ++++ | I-120 | ++++ |
| I-47 | ++++ | I-126 | ++++ |
| I-61 | ++++ | I-138 | ++++ |
| I-19 | ++++ | I-158 | ++++ |
| I-69 | ++++ | I-185 | ++++ |
| I-70 | ++++ | I-203 | +++ |
| I-72 | +++ | I-212 | ++++ |
| I-71 | ++ | I-216 | ++++ |
| I-73 | +++ | I-217 | ++++ |
| I-84 | ++++ | I-223 | ++++ |
| I-98 | ++++ | I-232 | ++++ |

### Example 68—Assay for Binding Affinity to BRD4-BD1

Exemplary compounds were tested for ability to bind to BRD4-BD1. Experimental procedures and results are provided below.

### Part I—Experimental Procedure

Compounds were tested using a bromoKdELECT assay. T7 phage strains displaying bromodomains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (5,000×g) and filtered (0.2 pm) to remove cell debris. Streptavidin-coated magnetic beads were treated with biotinylated small molecule or acetylated peptide ligands for 30 minutes at room temperature to generate affinity resins for bromodomain assays. The liganded beads were blocked with excess biotin and washed with blocking buffer (SeaBlock (Pierce), 1% BSA, 0.05% Tween 20, 1 mM DTT) to remove unbound ligand and to reduce nonspecific phage binding. Binding reactions were assembled by combining bromodomains, liganded affinity beads, and test compounds in 1× binding buffer (17% SeaBlock, 0.33×PBS, 0.04% Tween 20, 0.02% BSA, 0.004% Sodium azide, 7.4 mM DTT). Test compounds were prepared as 1000× stocks in 100% DMSO. Kds were determined using an 11-point 3-fold compound dilution series with one DMSO control point. All compounds for Kd measurements are distributed by acoustic transfer (non-contact dispensing) in 100% DMSO. The compounds were then diluted directly into the assays such that the final concentration of DMSO was 0.09%. All reactions performed in polypropylene 384-well plates. Each was a final volume of 0.02 ml. The assay plates were incubated at room temperature with shaking for 1 hour and the affinity beads were washed with wash buffer (1×PBS, 0.05% Tween 20). The beads were then resuspended in elution buffer (1×PBS, 0.05% Tween 20, 2 M non-biotinylated affinity ligand) and incubated at room temperature with shaking for 30 minutes. The bromodomain concentration in the eluates was measured by qPCR.

### Part II—Results

Results showing ability of exemplary compounds to bind to BRD4-BD1 are provided in Table 6 below. The symbol “++++” indicates a Kd less than 0.05 μM. The symbol “+++” indicates a Kd in the range of 0.05 μM to 0.5 μM. The symbol “++” indicates a Rd in the range of greater than 0.5 μM to 2.5 μM. The symbol “+” indicates a Kd greater than 2.5 μM. The symbol “N/A” indicates that no data was available.

TABLE 6

| Compound No. | Kd | Compound No. | Kd |
|---|---|---|---|
| I-1 | +++ | I-90 | + |
| I-3 | ++++ | I-91 | +++ |
| I-6 | + | I-92 | +++ |
| I-7 | ++ | I-96 | ++ |
| I-8 | +++ | I-94 | ++ |
| I-9 | +++ | I-99 | ++ |
| I-5 | +++ | I-105 | ++ |
| I-10 | +++ | I-106 | +++ |
| I-11 | +++ | I-107 | +++ |
| I-13 | ++ | I-108 | ++ |
| I-18 | ++ | I-109 | + |
| I-39 | ++++ | I-110 | ++ |
| I-62 | + | I-121 | + |
| I-47 | + | I-126 | +++ |
| I-48 | + | I-133 | +++ |
| I-49 | + | I-137 | + |
| I-51 | + | I-138 | + |
| I-52 | + | I-140 | + |
| I-61 | + | I-142 | + |
| I-64 | + | I-143 | +++ |
| I-75 | +++ | I-144 | +++ |
| I-54 | + | I-147 | + |
| I-56 | ++ | I-151 | +++ |
| I-68 | +++ | I-153 | ++ |
| I-65 | ++ | I-156 | ++ |
| I-84 | ++++ | I-185 | +++ |
| I-98 | + | I-186 | +++ |
| I-80 | +++ | I-187 | +++ |
| I-95 | ++ | I-190 | ++ |
| I-67 | +++ | I-192 | + |
| I-88 | ++ | I-193 | + |
| I-194 | + | I-211 | + |
| I-195 | + | I-214 | ++++ |
| I-196 | +++ | I-223 | ++++ |
| I-197 | +++ | I-228 | ++++ |
| I-203 | + | I-231 | ++++ |

### Example 69—Assay for Binding Affinity to BRD4-BD2

Exemplary compounds were tested for ability to bind to BRD4-BD2. Experimental procedures and results are provided below.

### Part I—Experimental Procedure

Compounds were tested using a bromoKdELECT assay. T7 phage strains displaying bromodomains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (5,000×g) and filtered (0.2 pm) to remove cell debris. Streptavidin-coated magnetic beads were treated with biotinylated small molecule or acetylated peptide ligands for 30 minutes at room temperature to generate affinity resins for bromodomain assays. The liganded beads were blocked with excess biotin and washed with blocking buffer (SeaBlock (Pierce), 1% BSA, 0.05% Tween 20, 1 mM DTT) to remove unbound ligand and to reduce nonspecific phage binding. Binding reactions were assembled by combining bromodomains, liganded affinity beads, and test compounds in 1× binding buffer (17% SeaBlock, 0.33×PBS, 0.04% Tween 20, 0.02% BSA, 0.004% Sodium azide, 7.4 mM DTT). Test compounds were prepared as 1000× stocks in 100% DMSO. Kds were determined using an 11-point 3-fold compound dilution series with one DMSO control point. All compounds for Kd measurements are distributed by acoustic transfer (non-contact dispensing) in 100% DMSO. The compounds were then diluted directly into the assays such that the final concentration of DMSO was 0.09%. All reactions performed in polypropylene 384-well plates. Each was a final volume of 0.02 ml. The assay plates were incubated at room temperature with shaking for 1 hour and the affinity beads were washed with wash buffer (1×PBS, 0.05% Tween 20). The beads were then resuspended in elution buffer (1×PBS, 0.05% Tween 20, 2 μM non-biotinylated affinity ligand) and incubated at room temperature with shaking for 30 minutes. The bromodomain concentration in the eluates was measured by qPCR.

### Part II—Results

Results showing ability of exemplary compounds to bind to BRD4-BD2 are provided in Table 7 below. The symbol “++++” indicates a Kd less than 0.05 μM. The symbol “+++” indicates a Kd in the range of 0.05 μM to 0.5 μM. The symbol “++” indicates a Kd in the range of greater than 0.5 μM to 2.5 μM. The symbol “+” indicates a Kd greater than 2.5 μM. The symbol “N/A” indicates that no data was available.

TABLE 7

| Compound No. | Kd | Compound No. | Kd |
|---|---|---|---|
| I-1 | ++++ | I-98 | + |
| I-3 | ++++ | I-80 | +++ |
| I-6 | +++ | I-95 | +++ |
| I-7 | ++++ | I-67 | +++ |
| I-8 | ++++ | I-88 | +++ |
| I-9 | ++++ | I-90 | + |
| I-5 | ++++ | I-91 | ++++ |
| I-10 | ++++ | I-92 | +++ |
| I-11 | ++++ | I-96 | +++ |
| I-13 | ++++ | I-94 | +++ |
| I-18 | ++++ | I-99 | ++++ |
| I-39 | ++++ | I-105 | ++++ |
| I-62 | + | I-106 | ++++ |
| I-47 | ++++ | I-107 | +++ |
| I-48 | ++ | I-108 | ++++ |
| I-49 | ++ | I-109 | ++ |
| I-51 | ++ | I-110 | ++++ |
| I-52 | ++ | I-121 | + |
| I-61 | + | I-126 | ++++ |
| I-64 | ++++ | I-133 | ++++ |
| I-75 | +++ | I-137 | + |
| I-54 | +++ | I-138 | ++ |
| I-56 | +++ | I-140 | + |
| I-68 | +++ | I-142 | ++ |
| I-65 | ++++ | I-143 | ++++ |
| I-84 | ++++ | I-144 | ++++ |
| I-147 | ++++ | I-194 | ++++ |
| I-151 | ++++ | I-195 | + |
| I-153 | ++++ | I-196 | ++++ |
| I-156 | +++ | I-197 | ++++ |
| I-185 | ++++ | I-203 | + |
| I-186 | +++ | I-211 | +++ |
| I-187 | ++ | I-214 | ++++ |
| I-190 | +++ | I-223 | ++++ |
| I-192 | +++ | I-228 | ++++ |
| I-193 | ++++ | I-231 | ++++ |

### Example 70—Cellular Growth Inhibition Assay Using T-Rex 293 Cells

Exemplary compounds were tested for ability to inhibit the proliferation of the following types of cells: (i) a T-Rex 293 cell line having increased expression of androgen receptor protein due to exposure to doxycycline and (ii) a T-Rex 293 cell line lacking increased expression of androgen receptor protein. Experimental procedures and results are provided below.

### Part I—Experimental Procedure

The following types of cells were prepared for this experiment: (i) a T-Rex 293 cell line having increased expression of androgen receptor protein due to exposure to doxycycline and (ii) a T-Rex 293 cell line lacking increased expression of androgen receptor protein. Ability of the test compounds to inhibit proliferation of the foregoing cell types was evaluated according to the procedures set forth below.

The doxycycline-inducible androgen receptor protein expressing cell line was established using the following protocol: T-Rex 293 cells were purchased from Invitrogen (Cat #R71007) and transfected using Lipofectamine 2000 with the wild-type androgen receptor protein sequence cloned into the pcDNA4/TO vector. Transfected cells were selected using 400 μg/mL Zeocin (Invitrogen Cat #R25001). Following selection, single clones were raised and maintained in Dulbecco's Modified Eagle Medium (DMEM) supplemented with 10% w/w Tetracycline-free fetal bovine serum (FBS) and 250 μg/mL Zeocin. Clones were analyzed for expression of androgen receptor protein in the presence and absence of 10 ng/mL doxycycline (Sigma Cat #D9891), and a single doxycycline-inducible clone (hereinafter “SC3”) was selected for use in downstream assays.

The SC3 cells were seeded on poly-D-lysine coated, black clear-bottom 384-well plates at 2500/well, in 25 μL Phenol Red Free Dulbecco's Modified Eagle Medium (DMEM) supplemented with 10% w/w charcoal-dextran treated fetal bovine serum (FBS) and 1% w/w pen-strep, with or without 10 ng/mL doxycycline. Pen-Strep is a commercially available mixture of penicillin G and streptomycin, which is used in mammalian cell culture media to prevent bacterial contamination. Phenol Red Free Dulbecco's Modified Eagle Medium (DMEM) supplemented with 10% w/w charcoal-dextran treated fetal bovine serum (FBS) and 1% w/w pen-strep, with or without 10 ng/mL doxycycline is herein referred to as Treatment Medium. Following seeding of cells in the plates, the plates were spun at 300×g for 30 seconds, then equilibrated to room temperature for 30 minutes, and then deposited in a humidified tissue culture incubator maintained at 37° C. with 5% CO₂.

At 24 hours after seeding of the cells, dilutions of test compound were prepared in DMSO and dissolved in Treatment Medium, to achieve a final DMSO concentration of 0.5% w/w, thereby providing the Test Compound Solution. A 25 μL aliquot of the Test Compound Solution was added to cells in the well plates. An equal volume of a solution containing DMSO and Treatment Medium was used as a negative control. Following treatment of cells with Test Compound Solution or said equal volume of a solution containing DMSO and Treatment Medium, the plates were spun at 300×g for 30 seconds, and then left in an incubator for 72 hours.

At the end of the treatment duration, cell viability was quantified with CellTiter-Glo 2.0 reagent (Promega Cat #G9243). For this purpose, plates were equilibrated to room temperature for 30 minutes, and then 25 μL of CellTiter-Glo 2.0 reagent was added to cells in the plate wells. Plates were then agitated on a shaker for two minutes at 500 rpm and subsequently incubated at room temperature for 10 minutes. Following incubation, the plates were spun at 3000×g for 30 seconds, then sealed with an optical adhesive cover, and luminescence readings were measured with an EnVision Plate Reader.

Data was normalized using zero luminescence for baseline. A four-parameter non-linear regression curve fit was applied to dose-response data in GraphPad Prism data analysis software to determine the half-maximal growth inhibitory concentration (GI₅₀) for each test compound.

### Part II—Results

The half-maximal growth inhibitory concentration (GI₅₀) results are provided in Tables 8 and 9 below for exemplary compounds. Table 8 provides results from the experiment analyzing ability of test compounds to inhibit proliferation of the T-Rex 293 cell line SC3 cells having increased expression of androgen receptor protein due to exposure to doxycycline. The symbol “++++” indicates a GI₅₀ less than 0.5 μM. The symbol “+++” indicates an GI₅₀ in the range of 0.5 μM to 1.5 μM. The symbol “++” indicates a GI₅₀ in the range of greater than 1.5 μM to 3 μM. The symbol “+” indicates a GI₅₀ greater than 3 μM. The symbol “N/A” indicates that no data was available.

TABLE 8

| Compound No. | GI₅₀ | Compound No. | GI₅₀ |
|---|---|---|---|
| I-1 | ++++ | I-15 | ++++ |
| I-3 | ++++ | I-16 | ++++ |
| I-2 | ++++ | I-24 | ++++ |
| I-4 | ++++ | I-25 | ++++ |
| I-6 | ++++ | I-26 | ++++ |
| I-7 | ++++ | I-27 | ++++ |
| I-8 | ++++ | I-28 | ++++ |
| I-9 | ++++ | I-30 | ++++ |
| I-5 | ++++ | I-41 | ++++ |
| I-10 | ++++ | I-42 | ++++ |
| I-11 | ++++ | I-43 | ++++ |
| I-14 | ++++ | I-62 | ++++ |
| I-13 | ++++ | I-63 | ++++ |
| I-18 | ++++ | I-66 | ++++ |
| I-23 | ++++ | I-20 | ++++ |
| I-35 | ++++ | I-22 | ++++ |
| I-39 | ++++ | I-47 | ++++ |
| I-44 | ++++ | I-48 | ++++ |
| I-45 | ++++ | I-49 | ++++ |
| I-50 | ++++ | I-84 | ++++ |
| I-57 | ++++ | I-97 | ++++ |
| I-58 | ++++ | I-98 | ++++ |
| I-59 | ++++ | I-80 | ++++ |
| I-60 | ++++ | I-86 | ++++ |
| I-12 | ++++ | I-93 | ++++ |
| I-17 | ++++ | I-95 | ++++ |
| I-29 | ++++ | I-67 | ++++ |
| I-31 | +++ | I-74 | ++++ |
| I-33 | ++++ | I-81 | ++++ |
| I-34 | ++++ | I-82 | ++++ |
| I-38 | ++++ | I-88 | ++++ |
| I-51 | ++++ | I-89 | ++++ |
| I-52 | ++++ | I-90 | ++++ |
| I-61 | ++++ | I-91 | ++++ |
| I-64 | ++++ | I-92 | ++++ |
| I-75 | ++++ | I-96 | ++++ |
| I-77 | ++++ | I-100 | ++++ |
| I-21 | ++++ | I-101 | ++++ |
| I-32 | ++++ | I-94 | ++++ |
| I-55 | ++++ | I-99 | ++++ |
| I-36 | ++++ | I-102 | ++++ |
| I-54 | ++++ | I-103 | ++++ |
| I-56 | ++++ | I-104 | ++++ |
| I-76 | ++++ | I-105 | ++++ |
| I-78 | ++++ | I-106 | ++++ |
| I-19 | ++++ | I-107 | ++++ |
| I-37 | ++++ | I-108 | ++++ |
| I-40 | ++++ | I-109 | ++++ |
| I-53 | ++++ | I-110 | ++++ |
| I-68 | ++++ | I-111 | ++++ |
| I-69 | ++++ | I-112 | ++++ |
| I-70 | ++++ | I-113 | ++++ |
| I-72 | ++++ | I-114 | ++++ |
| I-46 | ++++ | I-115 | ++++ |
| I-71 | ++++ | I-116 | ++++ |
| I-73 | ++++ | I-117 | ++++ |
| I-85 | ++++ | I-118 | ++++ |
| I-87 | ++++ | I-119 | ++++ |
| I-65 | ++++ | I-120 | ++++ |
| I-79 | ++++ | I-121 | ++++ |
| I-83 | ++++ | I-122 | ++++ |
| I-123 | ++++ | I-166 | ++++ |
| I-124 | ++++ | I-167 | ++++ |
| I-125 | ++++ | I-168 | ++++ |
| I-126 | ++++ | I-169 | ++++ |
| I-127 | ++++ | I-170 | ++++ |
| I-128 | ++++ | I-171 | ++++ |
| I-129 | ++++ | I-172 | ++++ |
| I-130 | ++++ | I-173 | +++ |
| I-131 | ++++ | I-174 | ++++ |
| I-132 | + | I-175 | ++++ |
| I-133 | ++++ | I-176 | ++++ |
| I-134 | ++++ | I-177 | ++++ |
| I-135 | ++++ | I-178 | ++++ |
| I-136 | ++++ | I-179 | ++++ |
| I-137 | ++++ | I-180 | ++++ |
| I-138 | ++++ | I-181 | ++++ |
| I-139 | ++++ | I-182 | ++++ |
| I-140 | ++++ | I-183 | +++- |
| I-142 | ++++ | I-184 | ++++ |
| I-143 | ++++ | I-185 | ++++ |
| I-144 | ++++ | I-186 | ++++ |
| I-145 | ++++ | I-187 | ++++ |
| I-146 | ++++ | I-188 | ++++ |
| I-147 | ++++ | I-189 | ++++ |
| I-148 | ++++ | I-190 | ++++ |
| I-149 | ++++ | I-191 | ++++ |
| I-150 | ++++ | I-192 | ++++ |
| I-151 | ++++ | I-193 | ++++ |
| I-152 | ++++ | I-194 | ++++ |
| I-153 | ++++ | I-195 | ++++ |
| I-154 | ++++ | I-196 | ++++ |
| I-155 | ++++ | I-197 | ++++ |
| I-156 | ++++ | I-198 | ++++ |
| I-157 | ++++ | I-199 | ++++ |
| I-158 | ++++ | I-200 | ++++ |
| I-159 | ++++ | I-201 | ++++ |
| I-160 | ++++ | I-202 | ++++ |
| I-161 | ++++ | I-203 | ++++ |
| I-162 | ++++ | I-204 | ++++ |
| I-163 | ++++ | I-205 | ++++ |
| I-164 | ++++ | I-206 | ++++ |
| I-165 | ++++ | I-207 | ++++ |
| I-208 | ++++ | I-255 | ++++ |
| I-209 | ++++ | I-257 | ++++ |
| I-210 | ++++ | I-258 | ++++ |
| I-211 | ++++ | I-259 | ++++ |
| I-212 | ++++ | I-260 | ++++ |
| I-213 | ++++ | I-261 | ++++ |
| I-214 | ++++ | I-262 | ++++ |
| I-215 | ++++ | I-263 | ++++ |
| I-216 | ++++ | I-264 | ++++ |
| I-217 | ++++ | I-265 | ++++ |
| I-218 | ++++ | I-266 | ++++ |
| I-219 | ++++ | I-267 | ++++ |
| I-220 | ++++ | I-268 | ++++ |
| I-221 | ++++ | I-269 | ++++ |
| I-222 | ++++ | I-270 | ++++ |
| I-223 | ++++ | I-271 | ++++ |
| I-224 | ++++ | I-272 | ++++ |
| I-225 | ++++ | I-273 | ++++ |
| I-226 | ++++ | I-274 | ++++ |
| I-227 | ++++ | I-275 | ++++ |
| I-228 | ++++ | I-276 | ++++ |
| I-229 | ++++ | I-277 | ++++ |
| I-230 | ++++ | I-278 | ++++ |
| I-231 | ++++ | I-279 | ++++ |
| I-232 | ++++ | I-280 | ++++ |
| I-233 | +++ | I-281 | ++++ |
| I-234 | ++++ | I-282 | ++++ |
| I-235 | ++++ | I-283 | ++++ |
| I-236 | ++++ | I-284 | ++++ |
| I-237 | ++++ | I-285 | ++++ |
| I-238 | ++++ | I-286 | ++++ |
| I-239 | ++++ | I-287 | ++++ |
| I-240 | ++++ | I-288 | ++++ |
| I-241 | ++++ | I-289 | ++++ |
| I-242 | ++++ | I-290 | ++++ |
| I-243 | ++++ | I-291 | ++++ |
| I-244 | ++++ | I-292 | ++++ |
| I-245 | ++++ | I-293 | ++++ |
| I-246 | ++++ | I-294 | ++++ |
| I-249 | ++++ | I-295 | ++++ |
| I-250 | ++++ | I-296 | ++++ |
| I-251 | ++++ | I-297 | ++++ |
| I-298 | ++++ | I-326 | ++++ |
| I-299 | ++++ | I-327 | ++++ |
| I-300 | ++++ | I-328 | ++++ |
| I-301 | ++++ | I-329 | ++++ |
| I-302 | ++++ | I-330 | ++++ |
| I-303 | ++++ | I-331 | ++++ |
| I-304 | ++++ | I-332 | + |
| I-305 | ++++ | I-333 | ++++ |
| I-306 | ++++ | I-334 | ++++ |
| I-307 | ++++ | I-335 | + |
| I-308 | ++++ | I-336 | ++++ |
| I-309 | ++++ | I-337 | ++++ |
| I-310 | ++++ | I-338 | + |
| I-311 | ++++ | I-339 | ++++ |
| I-312 | ++++ | I-340 | + |
| I-313 | +++ | I-341 | ++++ |
| I-314 | ++++ | I-342 | ++++ |
| I-315 | ++++ | I-343 | ++++ |
| I-316 | ++++ | I-344 | + |
| I-317 | ++++ | I-345 | ++++ |
| I-318 | ++++ | I-346 | ++++ |
| I-319 | ++++ | I-347 | + |
| I-320 | ++++ | I-348 | ++++ |
| I-321 | ++++ | I-349 | + |
| I-322 | ++++ | I-350 | ++++ |
| I-323 | +++ | I-351 | ++++ |
| I-324 | ++++ | I-352 | ++++ |
| I-325 | ++++ | I-353 | ++++ |

Table 9 provides results from the experiment analyzing ability of test compounds to inhibit proliferation of the T-Rex 293 cell line SC3 cells lacking increased expression of androgen receptor protein since such cells were not exposed to doxycycline. The symbol “++++” indicates a G150 less than 0.5 μM. The symbol “+++” indicates an G150 in the range of 0.5 μM to 1.5 μM. The symbol “++” indicates a GI₅₀ in the range of greater than 1.5 M to 3 μM. The symbol “+” indicates a GI₅₀ greater than 3 μM. The symbol “N/A” indicates that no data was available.

TABLE 9

| Compound No. | GI₅₀ | Compound No. | GI₅₀ |
|---|---|---|---|
| I-1 | +++ | I-58 | + |
| I-3 | + | I-59 | + |
| I-2 | ++ | I-60 | + |
| I-4 | ++++ | I-12 | ++++ |
| I-6 | ++++ | I-17 | ++++ |
| I-7 | ++++ | I-29 | + |
| I-8 | ++++ | I-31 | ++++ |
| I-9 | +++ | I-33 | ++++ |
| I-5 | +++ | I-34 | ++++ |
| I-10 | ++++ | I-38 | ++++ |
| I-11 | ++++ | I-51 | ++++ |
| I-14 | +++ | I-52 | ++++ |
| I-13 | ++++ | I-61 | ++ |
| I-18 | ++++ | I-64 | ++++ |
| I-23 | ++++ | I-75 | ++++ |
| I-35 | ++ | I-77 | ++++ |
| I-39 | ++++ | I-21 | ++++ |
| I-44 | + | I-32 | ++++ |
| I-45 | + | I-55 | ++++ |
| I-15 | +++ | I-36 | + |
| I-16 | ++++ | I-54 | ++++ |
| I-24 | ++++ | I-56 | ++++ |
| I-25 | ++++ | I-76 | ++ |
| I-26 | ++++ | I-78 | ++++ |
| I-27 | +++ | I-19 | ++++ |
| I-28 | +++ | I-37 | ++++ |
| I-30 | ++++ | I-40 | + |
| I-41 | +++ | I-53 | ++++ |
| I-42 | ++++ | I-68 | + |
| I-43 | ++ | I-69 | ++++ |
| I-62 | + | I-70 | ++++ |
| I-63 | + | I-72 | ++++ |
| I-66 | + | I-46 | + |
| I-20 | ++++ | I-71 | ++++ |
| I-22 | ++++ | I-73 | ++++ |
| I-47 | ++++ | I-85 | +++ |
| I-48 | + | I-87 | ++++ |
| I-49 | + | I-65 | ++++ |
| I-50 | ++++ | I-79 | +++ |
| I-57 | +++ | I-83 | + |
| I-84 | ++++ | I-123 | + |
| I-97 | +++ | I-124 | + |
| I-98 | +++ | I-125 | + |
| I-80 | ++++ | I-126 | + |
| I-86 | ++++ | I-127 | + |
| I-93 | + | I-128 | + |
| I-95 | ++++ | I-129 | ++++ |
| I-67 | ++++ | I-130 | + |
| I-74 | ++++ | I-131 | + |
| I-81 | + | I-132 | + |
| I-82 | + | I-133 | ++++ |
| I-88 | + | I-134 | +++ |
| I-89 | + | I-135 | + |
| I-90 | + | I-136 | ++++ |
| I-91 | + | I-137 | ++++ |
| I-92 | ++++ | I-138 | ++++ |
| I-96 | ++++ | I-139 | ++++ |
| I-100 | + | I-140 | ++++ |
| I-101 | + | I-142 | ++ |
| I-94 | + | I-143 | ++++ |
| I-99 | ++++ | I-144 | ++++ |
| I-102 | +++ | I-145 | + |
| I-103 | + | I-146 | + |
| I-104 | +++ | I-147 | ++++ |
| I-105 | ++++ | I-148 | + |
| I-106 | ++++ | I-149 | ++++ |
| I-107 | + | I-150 | + |
| I-108 | ++++ | I-151 | ++++ |
| I-109 | ++++ | I-152 | ++++ |
| I-110 | ++++ | I-153 | ++++ |
| I-111 | + | I-154 | ++++ |
| I-112 | + | I-155 | + |
| I-113 | ++++ | I-156 | + |
| I-114 | ++++ | I-157 | + |
| I-115 | ++++ | I-158 | + |
| I-116 | + | I-159 | ++++ |
| I-117 | + | I-160 | ++++ |
| I-118 | + | I-161 | + |
| I-119 | ++++ | I-162 | + |
| I-120 | + | I-163 | + |
| I-121 | + | I-164 | + |
| I-122 | + | I-165 | + |
| I-166 | ++++ | I-208 | ++++ |
| I-167 | + | I-209 | ++++ |
| I-168 | + | I-210 | ++++ |
| I-169 | ++++ | I-211 | ++++ |
| I-170 | + | I-212 | +++ |
| I-171 | + | I-213 | ++++ |
| I-172 | + | I-214 | ++++ |
| I-173 | + | I-215 | ++++ |
| I-174 | + | I-216 | ++++ |
| I-175 | ++++ | I-217 | ++++ |
| I-176 | ++++ | I-218 | ++ |
| I-177 | ++++ | I-219 | ++++ |
| I-178 | ++++ | I-220 | ++++ |
| I-179 | + | I-221 | + |
| I-180 | + | I-222 | +++ |
| I-181 | + | I-223 | ++++ |
| I-182 | + | I-224 | ++++ |
| I-183 | ++++ | I-225 | ++++ |
| I-184 | ++++ | I-226 | +++ |
| I-185 | ++++ | I-227 | ++++ |
| I-186 | ++++ | I-228 | ++++ |
| I-187 | + | I-229 | +++ |
| I-188 | ++++ | I-230 | +++ |
| I-189 | + | I-231 | ++++ |
| I-190 | +++ | I-232 | + |
| I-191 | ++++ | I-233 | + |
| I-192 | +++ | I-234 | ++++ |
| I-193 | ++++ | I-235 | ++++ |
| I-194 | ++ | I-236 | ++++ |
| I-195 | + | I-237 | +++ |
| I-196 | + | I-238 | +++ |
| I-197 | ++++ | I-239 | ++++ |
| I-198 | ++++ | I-240 | + |
| I-199 | ++++ | I-241 | ++++ |
| I-200 | +++ | I-242 | ++++ |
| I-201 | +++ | I-243 | +++ |
| I-202 | +++ | I-244 | +++ |
| I-203 | + | I-245 | + |
| I-204 | +++ | I-246 | ++++ |
| I-205 | ++++ | I-249 | + |
| I-206 | ++++ | I-250 | + |
| I-207 | ++++ | I-251 | +++ |
| I-255 | +++ | I-298 | ++++ |
| I-257 | ++++ | I-299 | + |
| I-258 | +++ | I-300 | ++++ |
| I-259 | +++ | I-301 | + |
| I-260 | ++++ | I-302 | +++ |
| I-261 | ++++ | I-303 | ++++ |
| I-262 | ++++ | I-304 | ++++ |
| I-263 | + | I-305 | + |
| I-264 | ++++ | I-306 | + |
| I-265 | +++ | I-307 | + |
| I-266 | ++ | I-308 | +++ |
| I-267 | +++ | I-309 | +++ |
| I-268 | +++ | I-310 | ++++ |
| I-269 | +++ | I-311 | +++ |
| I-270 | +++ | I-312 | +++ |
| I-271 | ++++ | I-313 | +++ |
| I-272 | ++++ | I-314 | + |
| I-273 | +++ | I-315 | + |
| I-274 | ++++ | I-316 | ++++ |
| I-275 | ++++ | I-317 | ++++ |
| I-276 | ++++ | I-318 | + |
| I-277 | ++++ | I-319 | ++++ |
| I-278 | + | I-320 | ++++ |
| I-279 | + | I-321 | ++++ |
| I-280 | ++ | I-322 | ++ |
| I-281 | +++ | I-323 | +++ |
| I-282 | +++ | I-324 | + |
| I-283 | + | I-325 | + |
| I-284 | + | I-326 | + |
| I-285 | + | I-327 | ++++ |
| I-286 | +++ | I-328 | ++++ |
| I-287 | + | I-329 | + |
| I-288 | ++++ | I-330 | ++++ |
| I-289 | ++ | I-331 | ++++ |
| I-290 | ++++ | I-332 | + |
| I-291 | ++++ | I-333 | + |
| I-292 | ++++ | I-334 | + |
| I-293 | + | I-335 | + |
| I-294 | + | I-336 | + |
| I-295 | ++++ | I-337 | + |
| I-296 | + | I-338 | + |
| I-297 | ++++ | I-339 | + |
| I-340 | + | I-347 | + |
| I-341 | ++++ | I-348 | + |
| I-342 | + | I-349 | + |
| I-343 | + | I-350 | ++++ |
| I-344 | + | I-351 | ++++ |
| I-345 | + | I-352 | + |
| I-346 | + | I-353 | ++ |

### Example 71—Cellular Growth Inhibition Assay for VCaP Cells

Exemplary compounds were tested for ability to inhibit the proliferation of VCaP cells. VCap cells are a commercially available human prostate cancer cell line. Experimental procedures and results are provided below.

### Part I—Experimental Procedure

VCaP cells were purchased from American Type Cell Culture (ATCC Cat #CRL2876) and then seeded on poly-D-lysine coated, black clear-bottom 384-well plates at 5000/well in 25 μL Dulbecco's Modified Eagle Medium (DMEM) supplemented with 10% w/w Fetal bovine serum (FBS) and 1% w/w Pen-Strep. Pen-Strep is a commercially available mixture of penicillin G and streptomycin, which is used in mammalian cell culture media to prevent bacterial contamination. Dulbecco's Modified Eagle Medium (DMEM) supplemented with 10% w/w fetal bovine serum (FBS) and 1% w/w Pen-Strep is herein referred to as Treatment Medium. Following seeding of cells in the plates, the plates were spun at 300×g for 30 seconds, then equilibrated to room temperature for 30 minutes, and then deposited in a humidified tissue culture incubator maintained at 37° C. with 5% CO₂.

At 24 hours after seeding of the cells, dilutions of test compound were prepared in DMSO and dissolved in Treatment Medium, to achieve a final DMSO concentration of 0.5% w/w, thereby providing the Test Compound Solution. A 25 μL aliquot of the Test Compound Solution was added to cells in the well plates. An equal volume of a solution containing DMSO and Treatment Medium was used as a negative control. Following treatment of cells with Test Compound Solution or said equal volume of a solution containing DMSO and Treatment Medium, the plates were spun at 300×g for 30 seconds, and then left in an incubator for 72 hours.

At the end of the treatment duration, cell viability was quantified with CellTiter-Glo 2.0 reagent (Promega Cat #G9243). For this purpose, plates were equilibrated to room temperature for 30 minutes, and then 25 μL of CellTiter-Glo 2.0 reagent was added to cells in the plate wells. Plates were then agitated on a shaker for two minutes at 500 rpm and subsequently incubated at room temperature for 10 minutes. Following incubation, the plates were spun at 3000×g for 30 seconds, then sealed with an optical adhesive cover, and luminescence readings were measured with an EnVision Plate Reader.

Data was normalized using zero luminescence for baseline. A four-parameter non-linear regression curve fit was applied to dose-response data in GraphPad Prism data analysis software to determine the half-maximal growth inhibitory concentration (GI₅₀) for each test compound.

### Part II—Results

The half-maximal growth inhibitory concentration (GI₅₀) results are provided in Table 10 below for exemplary compounds. The symbol “++++” indicates a GI₅₀ less than 0.5 μM. The symbol “+++” indicates an GI₅₀ in the range of 0.5 μM to 1.5 μM. The symbol “++” indicates a GI₅₀ in the range of greater than 1.5 μM to 3 μM. The symbol “+” indicates a GI₅₀ greater than 3 μM. The symbol “N/A” indicates that no data was available.

TABLE 10

| Compound No. | GI₅₀ | Compound No. | GI₅₀ |
|---|---|---|---|
| I-1 | ++++ | I-18 | ++++ |
| I-3 | ++++ | I-23 | ++++ |
| I-6 | ++++ | I-35 | ++++ |
| I-7 | ++++ | I-39 | ++++ |
| I-8 | ++++ | I-44 | ++++ |
| I-9 | ++++ | I-45 | ++++ |
| I-10 | ++++ | I-15 | ++++ |
| I-11 | ++++ | I-16 | ++++ |
| I-14 | ++++ | I-24 | ++++ |
| I-13 | ++++ | I-25 | ++++ |
| I-26 | ++++ | I-19 | ++++ |
| I-27 | ++++ | I-37 | ++++ |
| I-28 | ++++ | I-40 | ++++ |
| I-30 | ++++ | I-53 | ++++ |
| I-41 | ++++ | I-68 | ++++ |
| I-42 | ++++ | I-69 | ++++ |
| I-43 | ++++ | I-70 | ++++ |
| I-62 | ++++ | I-72 | ++++ |
| I-63 | ++++ | I-46 | ++++ |
| I-66 | ++++ | I-71 | ++++ |
| I-20 | ++++ | I-73 | ++++ |
| I-22 | ++++ | I-85 | ++++ |
| I-47 | ++++ | I-87 | ++++ |
| I-48 | ++++ | I-65 | ++++ |
| I-49 | ++++ | I-79 | ++++ |
| I-50 | ++++ | I-83 | ++++ |
| I-57 | ++++ | I-84 | ++++ |
| I-58 | ++++ | I-97 | ++++ |
| I-59 | + | I-98 | ++++ |
| I-60 | ++++ | I-80 | ++++ |
| I-12 | ++++ | I-86 | ++++ |
| I-17 | ++++ | I-93 | ++++ |
| I-29 | + | I-95 | ++++ |
| I-31 | ++++ | I-67 | ++++ |
| I-33 | ++++ | I-74 | ++++ |
| I-34 | ++++ | I-81 | ++++ |
| I-38 | ++++ | I-82 | ++++ |
| I-51 | ++++ | I-88 | ++++ |
| I-52 | ++++ | I-89 | ++++ |
| I-61 | ++++ | I-90 | ++++ |
| I-64 | ++++ | I-91 | ++++ |
| I-75 | ++++ | I-92 | ++++ |
| I-77 | ++++ | I-96 | ++++ |
| I-21 | ++++ | I-100 | ++++ |
| I-32 | ++++ | I-101 | ++++ |
| I-55 | ++++ | I-94 | + |
| I-36 | ++++ | I-99 | ++++ |
| I-54 | ++++ | I-102 | ++++ |
| I-56 | ++++ | I-103 | ++++ |
| I-76 | ++++ | I-104 | ++++ |
| I-78 | ++++ | I-105 | ++++ |
| I-106 | ++++ | I-148 | ++++ |
| I-107 | + | I-149 | ++++ |
| I-108 | ++++ | I-150 | ++++ |
| I-109 | ++++ | I-151 | ++++ |
| I-110 | ++++ | I-152 | ++++ |
| I-111 | ++++ | I-153 | ++++ |
| I-112 | + | I-154 | ++++ |
| I-113 | ++++ | I-155 | ++++ |
| I-114 | ++++ | I-156 | + |
| I-115 | ++++ | I-157 | ++++ |
| I-116 | ++++ | I-158 | ++++ |
| I-117 | ++++ | I-159 | ++++ |
| I-118 | ++++ | I-160 | ++++ |
| I-119 | ++++ | I-161 | ++++ |
| I-120 | ++++ | I-162 | ++++ |
| I-121 | ++++ | I-163 | +++ |
| I-122 | + | I-164 | ++++ |
| I-123 | + | I-165 | ++++ |
| I-124 | ++++ | I-166 | ++++ |
| I-125 | ++++ | I-167 | ++++ |
| I-126 | ++++ | I-168 | ++++ |
| I-127 | ++++ | I-169 | ++++ |
| I-128 | ++++ | I-170 | ++++ |
| I-129 | ++++ | I-171 | + |
| I-130 | ++++ | I-172 | + |
| I-131 | ++++ | I-173 | ++++ |
| I-132 | + | I-174 | ++++ |
| I-133 | ++++ | I-175 | ++++ |
| I-134 | ++++ | I-176 | ++++ |
| I-135 | ++++ | I-177 | ++++ |
| I-136 | ++++ | I-178 | ++++ |
| I-137 | + | I-179 | + |
| I-138 | ++++ | I-180 | ++++ |
| I-139 | ++++ | I-181 | ++++ |
| I-140 | ++ | I-182 | ++++ |
| I-142 | ++++ | I-183 | ++++ |
| I-143 | ++++ | I-184 | ++++ |
| I-144 | ++++ | I-185 | ++++ |
| I-145 | ++ | I-186 | ++++ |
| I-146 | ++++ | I-187 | ++++ |
| I-147 | ++++ | I-188 | ++++ |
| I-189 | + | I-230 | ++++ |
| I-190 | ++++ | I-231 | ++++ |
| I-191 | ++++ | I-232 | +++ |
| I-192 | +++ | I-233 | ++++ |
| I-193 | ++++ | I-234 | ++++ |
| I-194 | +++ | I-235 | ++++ |
| I-195 | + | I-236 | ++++ |
| I-196 | + | I-237 | +++ |
| I-197 | ++++ | I-238 | ++++ |
| I-198 | ++++ | I-239 | ++++ |
| I-199 | ++++ | I-240 | +++ |
| I-200 | ++++ | I-241 | ++++ |
| I-201 | +++ | I-242 | ++++ |
| I-202 | +++ | I-243 | ++++ |
| I-203 | ++ | I-244 | ++++ |
| I-204 | ++++ | I-245 | +++ |
| I-205 | ++++ | I-246 | ++++ |
| I-206 | ++++ | I-247 | ++++ |
| I-207 | ++++ | I-248 | +++ |
| I-208 | +++ | I-249 | +++ |
| I-209 | ++++ | I-250 | +++ |
| I-210 | ++++ | I-251 | +++ |
| I-211 | ++++ | I-252 | ++++ |
| I-212 | ++ | I-253 | ++++ |
| I-213 | ++++ | I-254 | ++++ |
| I-214 | ++++ | I-255 | ++++ |
| I-215 | ++++ | I-256 | ++++ |
| I-216 | ++++ | I-257 | ++++ |
| I-217 | ++++ | I-258 | ++++ |
| I-218 | ++ | I-259 | ++++ |
| I-219 | ++++ | I-260 | +++ |
| I-220 | + | I-261 | ++++ |
| I-221 | + | I-262 | ++++ |
| I-222 | ++++ | I-263 | ++++ |
| I-223 | ++++ | I-264 | ++++ |
| I-224 | ++++ | I-265 | +++ |
| I-225 | ++++ | I-266 | ++++ |
| I-226 | +++ | I-267 | ++++ |
| I-227 | ++++ | I-268 | ++++ |
| I-228 | ++++ | I-269 | +++ |
| I-229 | ++++ | I-270 | ++++ |
| I-271 | ++++ | I-312 | ++++ |
| I-272 | ++++ | I-313 | ++++ |
| I-273 | ++++ | I-314 | ++++ |
| I-274 | ++++ | I-315 | ++++ |
| I-275 | ++++ | I-316 | ++++ |
| I-276 | ++++ | I-317 | ++++ |
| I-277 | ++++ | I-318 | + |
| I-278 | ++++ | I-319 | ++++ |
| I-279 | ++++ | I-320 | ++++ |
| I-280 | ++++ | I-321 | ++++ |
| I-281 | ++++ | I-322 | +++ |
| I-282 | ++++ | I-323 | ++++ |
| I-283 | ++++ | I-324 | ++++ |
| I-284 | ++++ | I-325 | + |
| I-285 | ++++ | I-326 | ++++ |
| I-286 | ++++ | I-327 | ++++ |
| I-287 | +++ | I-328 | ++++ |
| I-288 | ++++ | I-329 | ++++ |
| I-289 | +++ | I-330 | ++++ |
| I-290 | ++++ | I-331 | ++++ |
| I-291 | ++++ | I-332 | ++++ |
| I-292 | ++++ | I-333 | ++++ |
| I-293 | ++++ | I-334 | ++++ |
| I-294 | ++++ | I-335 | +++ |
| I-295 | ++++ | I-336 | + |
| I-296 | ++++ | I-337 | ++++ |
| I-297 | ++++ | I-338 | + |
| I-298 | ++++ | I-339 | + |
| I-299 | ++++ | I-340 | + |
| I-300 | ++++ | I-341 | ++++ |
| I-301 | +++ | I-342 | ++++ |
| I-302 | ++++ | I-343 | ++++ |
| I-303 | ++++ | I-344 | + |
| I-304 | ++++ | I-345 | ++++ |
| I-305 | ++++ | I-346 | ++++ |
| I-306 | +++ | I-347 | ++++ |
| I-307 | ++++ | I-348 | ++++ |
| I-308 | ++++ | I-349 | ++++ |
| I-309 | ++++ | I-350 | ++++ |
| I-310 | ++++ | I-351 | ++++ |
| I-311 | ++++ | I-352 | ++++ |
| I-353 | ++ | | |

### Example 72—Cellular Growth Inhibition Assay Using T-Rex 293 Cells

Exemplary compounds were tested for ability to inhibit the proliferation of the following types of cells: (i) a T-Rex 293 cell line having increased expression of progesterone receptor isoform B protein due to exposure of doxycycline and (ii) a T-Rex 293 cell line lacking increased expression of progesterone receptor isoform B protein. Experimental procedures and results are provided below.

### Part I—Experimental Procedure

The following types of cells were prepared for this experiment: (i) a T-Rex 293 cell line having increased expression of progesterone receptor isoform B protein due to exposure to doxycycline and (ii) a T-Rex 293 cell line lacking increased expression of progesterone receptor isoform B protein. Ability of the test compounds to inhibit proliferation of the foregoing cell types was evaluated according to the procedures set forth below.

The doxycycline-inducible progesterone receptor isoform B protein expressing cell line was established using the following protocol: T-Rex 293 cells were purchased from Invitrogen (Cat #R71007) and transfected using Lipofectamine 2000 with the wild-type progesterone receptor B protein sequence cloned into the pcDNA4/TO vector. Transfected cells were selected using 400 μg/mL Zeocin (Invitrogen Cat #R25001). Following selection, single clones were raised and maintained in Dulbecco's Modified Eagle Medium (DMEM) supplemented with 10% w/w Tetracycline-free fetal bovine serum (FBS) and 250 μg/mL Zeocin. Clones were analyzed for expression of progesterone receptor isoform B protein in the presence and absence of 10 ng/mL doxycycline (Sigma Cat #D9891), and a single doxycycline-inducible clone (hereinafter “SC”) was selected for use in downstream assays.

The SC cells were seeded on poly-D-lysine coated, black clear-bottom 384-well plates at 2500/well, in 25 μL Phenol Red Free Dulbecco's Modified Eagle Medium (DMEM) supplemented with 10% w/w charcoal-dextran treated fetal bovine serum (FBS) and 1% w/w pen-strep, with or without 10 ng/mL doxycycline. Pen-Strep is a commercially available mixture of penicillin G and streptomycin, which is used in mammalian cell culture media to prevent bacterial contamination. Phenol Red Free Dulbecco's Modified Eagle Medium (DMEM) supplemented with 10% w/w charcoal-dextran treated fetal bovine serum (FBS) and 1% w/w pen-strep, with or without 10 ng/mL doxycycline is herein referred to as Treatment Medium. Following seeding of cells in the plates, the plates were spun at 300×g for 30 seconds, then equilibrated to room temperature for 30 minutes, and then deposited in a humidified tissue culture incubator maintained at 37° C. with 5% CO₂.

At 24 hours after seeding of the cells, dilutions of test compound were prepared in DMSO and dissolved in Treatment Medium, to achieve a final DMSO concentration of 0.5% w/w, thereby providing the Test Compound Solution. A 25 μL aliquot of the Test Compound Solution was added to cells in the well plates. An equal volume of a solution containing DMSO and Treatment Medium was used as a negative control. Following treatment of cells with Test Compound Solution or said equal volume of a solution containing DMSO and Treatment Medium, the plates were spun at 300×g for 30 seconds, and then left in an incubator for 72 hours.

At the end of the treatment duration, cell viability was quantified with CellTiter-Glo 2.0 reagent (Promega Cat #G9243). For this purpose, plates were equilibrated to room temperature for 30 minutes, and then 25 μL of CellTiter-Glo 2.0 reagent was added to cells in the plate wells. Plates were then agitated on a shaker for two minutes at 500 rpm and subsequently incubated at room temperature for 10 minutes. Following incubation, the plates were spun at 3000×g for 30 seconds, then sealed with an optical adhesive cover, and luminescence readings were measured with an EnVision Plate Reader (Perkin Elmer).

Data was normalized using zero luminescence for baseline. A four-parameter non-linear regression curve fit was applied to dose-response data in GraphPad Prism data analysis software to determine the half-maximal growth inhibitory concentration (GI₅₀) for each test compound.

### Part II—Results

The half-maximal growth inhibitory concentration (GI₅₀) results are provided in Tables 11 and 12 below for exemplary compounds. Table 11 provides results from the experiment analyzing ability of test compounds to inhibit proliferation of the T-Rex 293 cell line SC cells having increased expression of progesterone receptor isoform B protein due to exposure to doxycycline. The symbol “++++” indicates a GI₅₀ less than 0.5 μM. The symbol “+++” indicates an GI₅₀ in the range of 0.5 M to 1.5 μM. The symbol “++” indicates a GI₅₀ in the range of greater than 1.5 μM to 3 μM. The symbol “+” indicates a GI₅₀ greater than 3 M. The symbol “N/A” indicates that no data was available.

TABLE 11

| Compound No. | GI₅₀ | Compound No. | GI₅₀ |
|---|---|---|---|
| I-1 | ++++ | I-202 | ++++ |
| I-3 | ++++ | I-203 | ++++ |
| I-6 | ++++ | I-204 | ++++ |
| I-7 | ++++ | I-205 | ++++ |
| I-18 | ++++ | I-206 | ++++ |
| I-35 | ++++ | I-207 | ++++ |
| I-44 | ++++ | I-208 | ++++ |
| I-45 | ++++ | I-213 | ++++ |
| I-27 | ++++ | I-219 | ++++ |
| I-36 | ++++ | I-220 | ++++ |
| I-70 | ++++ | I-221 | ++++ |
| I-71 | ++++ | I-228 | ++++ |
| I-65 | ++++ | I-232 | ++++ |
| I-92 | ++++ | I-238 | ++++ |
| I-104 | ++++ | I-255 | ++++ |
| I-138 | ++++ | I-268 | ++++ |
| I-175 | ++++ | I-279 | ++++ |
| I-190 | ++++ | I-280 | ++++ |
| I-195 | ++++ | I-299 | ++++ |
| I-196 | ++++ | I-303 | ++++ |
| I-197 | ++++ | I-304 | ++++ |
| I-198 | ++++ | I-317 | ++++ |
| I-199 | ++++ | I-322 | ++++ |
| I-200 | ++++ | I-324 | ++++ |
| I-201 | ++++ | | |

Table 12 provides results from the experiment analyzing ability of test compounds to inhibit proliferation of the T-Rex 293 cell line SC cells lacking increased expression of progesterone receptor isoform B protein since such cells were not exposed to doxycycline. The symbol “++++” indicates a GI₅₀ less than 0.5 μM. The symbol “+++” indicates an GI₅₀ in the range of 0.5 μM to 1.5 μM. The symbol “++” indicates a GI₅₀ in the range of greater than 1.5 μM to 3 μM. The symbol “+” indicates a GI₅₀ greater than 3 μM. The symbol “N/A” indicates that no data was available.

TABLE 12

| Compound No. | GI₅₀ | Compound No. | GI₅₀ |
|---|---|---|---|
| I-1 | ++++ | I-202 | +++ |
| I-3 | +++ | I-203 | + |
| I-6 | ++++ | I-204 | +++ |
| I-7 | ++++ | I-205 | ++++ |
| I-18 | ++++ | I-206 | ++++ |
| I-35 | + | I-207 | ++++ |
| I-44 | + | I-208 | ++++ |
| I-45 | + | I-213 | ++++ |
| I-27 | + | I-219 | + |
| I-36 | + | I-220 | ++++ |
| I-70 | ++++ | I-221 | ++ |
| I-71 | ++++ | I-228 | ++++ |
| I-65 | ++++ | I-232 | +++ |
| I-92 | ++++ | I-238 | ++++ |
| I-104 | + | I-255 | ++ |
| I-138 | ++++ | I-268 | ++++ |
| I-175 | + | I-279 | + |
| I-190 | ++++ | I-280 | ++++ |
| I-195 | + | I-299 | +++ |
| I-196 | + | I-303 | +++ |
| I-197 | ++++ | I-304 | +++ |
| I-198 | ++++ | I-317 | +++ |
| I-199 | ++++ | I-322 | ++ |
| I-200 | ++++ | I-324 | + |
| I-201 | +++ | | |

### Example 73—Cellular Growth Inhibition Assay for T47D Cells

Exemplary compounds were tested for ability to inhibit the proliferation of T47D cells. T47D cells are a commercially available human breast cancer cell line. Experimental procedures and results are provided below.

### Part I—Experimental Procedure

T47D cells were purchased from American Type Cell Culture (ATCC Cat #HTB-133) and then seeded on poly-D-lysine coated, black clear-bottom 384-well plates at 3000/well in 25 μL Dulbecco's Modified Eagle Medium (DMEM) supplemented with 10% w/w Fetal bovine serum (FBS), 0.2 units/mL recombinant human insulin (Gibco Cat #12585014), and 1% w/w Pen-Strep. Pen-Strep is a commercially available mixture of penicillin G and streptomycin, which is used in mammalian cell culture media to prevent bacterial contamination. Dulbecco's Modified Eagle Medium (DMEM) supplemented with 10% w/w fetal bovine serum (FBS), 0.2 units/mL recombinant human insulin, and 1% w/w Pen-Strep is herein referred to as Treatment Medium. Following seeding of cells in the plates, the plates were spun at 300×g for 30 seconds, then equilibrated to room temperature for 30 minutes, and then deposited in a humidified tissue culture incubator maintained at 37° C. with 5% CO₂.

At 24 hours after seeding of the cells, dilutions of test compound were prepared in DMSO and dissolved in Treatment Medium, to achieve a final DMSO concentration of 0.5% w/w, thereby providing the Test Compound Solution. A 25 μL aliquot of the Test Compound Solution was added to cells in the well plates. An equal volume of a solution containing DMSO and Treatment Medium was used as a negative control. Following treatment of cells with Test Compound Solution or said equal volume of a solution containing DMSO and Treatment Medium, the plates were spun at 300×g for 30 seconds, and then left in an incubator for 72 hours.

At the end of the treatment duration, cell viability was quantified with CellTiter-Glo 2.0 reagent (Promega Cat #G9243). For this purpose, plates were equilibrated to room temperature for 30 minutes, and then 25 μL of CellTiter-Glo 2.0 reagent was added to cells in the plate wells. Plates were then agitated on a shaker for two minutes at 500 rpm and subsequently incubated at room temperature for 10 minutes. Following incubation, the plates were spun at 3000×g for 30 seconds, then sealed with an optical adhesive cover, and luminescence readings were measured with an EnVision Plate Reader (Perkin Elmer).

Data was normalized using zero luminescence for baseline. A four-parameter non-linear regression curve fit was applied to dose-response data in GraphPad Prism data analysis software to determine the half-maximal growth inhibitory concentration (GI₅₀) for each test compound.

### Part II—Results

The half-maximal growth inhibitory concentration (GI₅₀) results are provided in Table 13 below for exemplary compounds. The symbol “++++” indicates a GI₅₀ less than 0.5 μM. The symbol “+++” indicates an GI₅₀ in the range of 0.5 μM to 1.5 μM. The symbol “++” indicates a GI₅₀ in the range of greater than 1.5 μM to 3 μM. The symbol “+” indicates a GI₅₀ greater than 3 μM. The symbol “N/A” indicates that no data was available.

TABLE 13

| Compound No. | GI₅₀ | Compound No. | GI₅₀ |
|---|---|---|---|
| I-6 | ++++ | I-78 | ++++ |
| I-7 | ++++ | I-37 | +++ |
| I-18 | ++++ | I-40 | ++++ |
| I-23 | ++++ | I-70 | ++++ |
| I-35 | ++++ | I-71 | ++++ |
| I-44 | ++++ | I-65 | ++++ |
| I-45 | ++++ | I-79 | ++++ |
| I-24 | ++++ | I-83 | ++++ |
| I-25 | ++++ | I-84 | ++++ |
| I-27 | ++++ | I-97 | +++ |
| I-30 | ++++ | I-98 | ++++ |
| I-41 | ++++ | I-80 | ++++ |
| I-42 | ++++ | I-93 | +++ |
| I-43 | ++++ | I-74 | ++++ |
| I-62 | +++ | I-81 | ++++ |
| I-63 | +++ | I-82 | ++++ |
| I-57 | ++++ | I-88 | +++ |
| I-58 | +++ | I-89 | +++ |
| I-31 | ++++ | I-90 | +++ |
| I-61 | ++++ | I-91 | +++ |
| I-75 | ++++ | I-92 | ++++ |
| I-36 | ++++ | I-96 | +++ |
| I-54 | ++++ | I-100 | ++ |
| I-56 | ++++ | I-101 | +++ |
| I-76 | ++++ | I-94 | +++ |
| I-99 | +++ | I-190 | +++ |
| I-102 | ++ | I-191 | ++++ |
| I-103 | ++ | I-192 | +++ |
| I-104 | ++++ | I-193 | ++++ |
| I-105 | +++ | I-194 | +++ |
| I-107 | ++++ | I-195 | +++ |
| I-110 | ++++ | I-196 | +++ |
| I-114 | ++++ | I-197 | ++++ |
| I-115 | +++ | I-198 | ++++ |
| I-117 | +++ | I-199 | ++++ |
| I-118 | ++++ | I-200 | +++ |
| I-119 | ++++ | I-201 | ++++ |
| I-120 | ++++ | I-202 | +++ |
| I-121 | +++ | I-203 | +++ |
| I-122 | +++ | I-204 | ++++ |
| I-123 | +++ | I-205 | ++++ |
| I-125 | ++ | I-206 | ++++ |
| I-126 | ++++ | I-208 | ++++ |
| I-129 | ++++ | I-209 | ++++ |
| I-134 | +++ | I-210 | +++ |
| I-137 | ++++ | I-211 | ++++ |
| I-138 | ++++ | I-212 | +++ |
| I-141 | +++ | I-213 | ++++ |
| I-142 | +++ | I-214 | ++++ |
| I-143 | +++ | I-215 | ++++ |
| I-146 | ++++ | I-216 | ++++ |
| I-147 | ++++ | I-217 | ++++ |
| I-148 | +++ | I-218 | +++ |
| I-149 | +++ | I-219 | ++++ |
| I-156 | +++ | I-220 | ++++ |
| I-157 | ++++ | I-221 | +++ |
| I-158 | ++++ | I-222 | +++ |
| I-164 | ++++ | I-223 | ++++ |
| I-165 | ++++ | I-224 | ++++ |
| I-170 | +++ | I-225 | ++++ |
| I-171 | ++++ | I-226 | + |
| I-175 | ++++ | I-227 | ++++ |
| I-178 | ++++ | I-228 | ++++ |
| I-179 | +++ | I-229 | ++++ |
| I-180 | ++++ | I-230 | ++++ |
| I-181 | ++++ | I-231 | ++++ |
| I-182 | ++++ | I-232 | ++++ |
| I-189 | ++++ | I-233 | +++ |
| I-234 | ++++ | I-277 | ++++ |
| I-235 | ++++ | I-278 | ++++ |
| I-236 | ++++ | I-279 | ++++ |
| I-237 | +++ | I-280 | ++++ |
| I-238 | ++++ | I-281 | ++++ |
| I-239 | ++++ | I-282 | +++ |
| I-240 | ++++ | I-283 | ++++ |
| I-241 | ++++ | I-284 | ++++ |
| I-242 | ++++ | I-285 | ++++ |
| I-243 | +++ | I-286 | ++++ |
| I-244 | +++ | I-287 | ++++ |
| I-245 | ++++ | I-288 | ++++ |
| I-246 | +++ | I-289 | ++ |
| I-247 | ++++ | I-290 | +++ |
| I-248 | ++++ | I-291 | ++++ |
| I-249 | +++ | I-292 | ++++ |
| I-250 | ++++ | I-293 | ++++ |
| I-251 | +++ | I-294 | ++++ |
| I-252 | +++ | I-295 | ++++ |
| I-253 | +++ | I-296 | + |
| I-254 | + | I-297 | ++++ |
| I-255 | ++++ | I-298 | ++++ |
| I-256 | ++++ | I-299 | ++++ |
| I-257 | ++++ | I-300 | ++++ |
| I-258 | ++++ | I-301 | ++++ |
| I-259 | ++++ | I-302 | +++ |
| I-260 | +++ | I-303 | ++++ |
| I-261 | ++++ | I-304 | ++++ |
| I-262 | ++++ | I-305 | ++++ |
| I-263 | ++++ | I-306 | + |
| I-264 | ++++ | I-307 | ++++ |
| I-265 | ++ | I-308 | + |
| I-266 | +++ | I-309 | ++++ |
| I-267 | +++ | I-310 | ++++ |
| I-268 | ++++ | I-311 | ++++ |
| I-269 | +++ | I-312 | +++ |
| I-270 | ++++ | I-313 | ++++ |
| I-271 | ++++ | I-314 | +++ |
| I-272 | ++++ | I-315 | +++ |
| I-273 | ++++ | I-316 | +++ |
| I-274 | ++++ | I-317 | ++++ |
| I-275 | ++++ | I-318 | ++++ |
| I-276 | ++++ | I-319 | ++++ |
| I-320 | ++++ | I-359 | + |
| I-321 | ++++ | I-360 | +++ |
| I-322 | ++++ | I-361 | ++++ |
| I-323 | ++++ | I-362 | ++++ |
| I-324 | ++++ | I-363 | ++++ |
| I-325 | ++++ | I-364 | + |
| I-326 | ++++ | I-365 | ++++ |
| I-327 | ++++ | I-366 | + |
| I-328 | ++++ | I-367 | + |
| I-329 | + | I-368 | ++++ |
| I-330 | ++++ | I-369 | ++++ |
| I-331 | ++++ | I-370 | ++++ |
| I-332 | ++++ | I-371 | ++++ |
| I-333 | ++++ | I-372 | ++++ |
| I-334 | ++++ | I-373 | ++++ |
| I-335 | ++++ | I-374 | +++ |
| I-336 | ++++ | I-375 | + |
| I-337 | ++++ | I-376 | ++++ |
| I-338 | +++ | I-377 | + |
| I-339 | ++++ | I-378 | + |
| I-340 | ++++ | I-379 | ++ |
| I-341 | ++++ | I-380 | +++ |
| I-342 | ++++ | I-381 | +++ |
| I-343 | ++++ | I-382 | ++++ |
| I-344 | +++ | I-383 | ++++ |
| I-345 | ++++ | I-384 | ++++ |
| I-346 | ++++ | I-385 | ++++ |
| I-347 | ++++ | I-386 | ++++ |
| I-348 | ++++ | I-387 | + |
| I-349 | ++++ | I-388 | ++++ |
| I-350 | ++++ | I-389 | ++++ |
| I-351 | ++++ | I-390 | ++++ |
| I-354 | ++++ | I-391 | ++++ |
| I-355 | ++++ | I-392 | ++++ |
| I-356 | ++++ | I-393 | ++++ |
| I-357 | ++++ | I-394 | ++++ |
| I-358 | + | | |

### Example 74—Assay for Binding Affinity to Progesterone Receptor

Exemplary compounds were tested for ability to bind to the progesterone receptor. Experimental procedures and results are provided below.

### Part I—Experimental Procedure

Compounds were tested using the PR Human Progesterone NHR Binding (Agonist Radioligand) Assay, Cerep at Eurofins Discovery using a modified version of the protocol from Sarup et al. (Cancer Res 1988; 48:5071-5078). Briefly, cytosolic fractions of T47D cells were incubated with 0.5 nM [³H]progesterone for 1 hour at 4° C. in the absence or presence of the test compound. Nonspecific binding was determined in the presence of 1 M promegestone. Reactions were subjected to scintillation counting. Results are expressed as a percent inhibition of the control radioligand specific binding.

### Part II—Results

Results showing ability of exemplary compounds to bind to the progesterone receptor are provided in Table 14 below. The symbol “++++” indicates a Ki less than 0.05 μM. The symbol “+++” indicates an Ki in the range of 0.05 μM to 0.5 μM. The symbol “++” indicates a Ki in the range of greater than 0.5 M to 2.5 μM. The symbol “+” indicates a Ki greater than 2.5 μM. The symbol “N/A” indicates that no data was available.

TABLE 14

| Compound No. | Ki |
|---|---|
| I-19 | ++++ |
| I-185 | ++++ |
| I-232 | ++++ |
| I-332 | +++ |
| I-373 | ++ |

### Example 75—FP Assay for Binding Affinity to Progesterone Receptor

Exemplary compounds were tested for ability to bind to the progesterone receptor ligand binding domain (LBD). Experimental procedures and results are provided below.

### Part I—Experimental Procedure

Compounds were tested using the PolarScreen™ Progesterone Receptor Competitor Assay Kit (Life Technologies Cat #A15905 or a15906), as per the manufacturer's instructions. Briefly, GST-tagged PR LBD, Fluormone tracer, and compound were mixed and incubated for 2 hours at room temperature in the dark. Following incubation, the plates were spun at 3000×g for 30 seconds, then sealed with an optical adhesive cover, and fluorescence polarization was measured using appropriate excitation and emission filters with an EnVision Plate Reader (Perkin Elmer). Compound concentration vs. mP value were plotted in Prism (GraphPad). A four-parameter non-linear regression curve fit was applied to dose-response data to determine the half-maximal inhibitory concentration (IC₅₀) for each test compound.

### Part II—Results

The half-maximal inhibitory concentration (IC₅₀) results are provided in Table 15 below for exemplary compounds. The symbol “++++” indicates a IC₅₀ less than 0.05 μM. The symbol “+++” indicates an IC₅₀ in the range of 0.05 μM to 0.5 μM. The symbol “++” indicates a IC₅₀ in the range of greater than 0.5 μM to 2.5 μM. The symbol “+” indicates a IC₅₀ greater than 2.5 μM. The symbol “N/A” indicates that no data was available.

TABLE 15

| Compound No. | IC₅₀ | Compound No. | IC₅₀ |
|---|---|---|---|
| I-35 | ++ | I-219 | +++ |
| I-45 | + | I-220 | +++ |
| I-83 | +++ | I-221 | +++ |
| I-80 | + | I-222 | ++ |
| I-74 | + | I-223 | +++ |
| I-99 | +++ | I-224 | +++ |
| I-104 | + | I-225 | ++++ |
| I-138 | + | I-226 | ++++ |
| I-201 | + | I-227 | ++++ |
| I-202 | + | I-228 | +++ |
| I-203 | + | I-229 | +++ |
| I-204 | +++ | I-232 | ++ |
| I-208 | +++ | I-233 | + |
| I-212 | +++ | I-238 | +++ |
| I-214 | ++++ | I-245 | ++ |
| I-215 | + | I-255 | +++ |
| I-216 | +++ | I-268 | +++ |
| I-217 | ++++ | I-280 | +++ |
| I-218 | +++ | | |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A compound represented by Formula I: 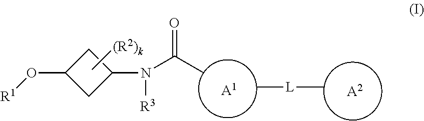
or a pharmaceutically acceptable salt thereof; wherein:
R¹ is phenyl substituted by cyano, halogen, and m occurrences of R⁴;
R² represents independently for each occurrence C₁₋₄ alkyl;
R³ is hydrogen or C₁₋₄ alkyl;
R⁴ is C₁₋₄ alkyl;
R⁵ represents independently for each occurrence C₁₋₄ alkyl or halogen, or one occurrence of R⁵ is taken together with R³ to form a C₁₋₃ alkylene;
A¹ is a pyridazinylene, pyrimidinylene, pyrazinylene, pyridinylene, or phenylene, each of which is substituted with n occurrences of R⁵;
A² is 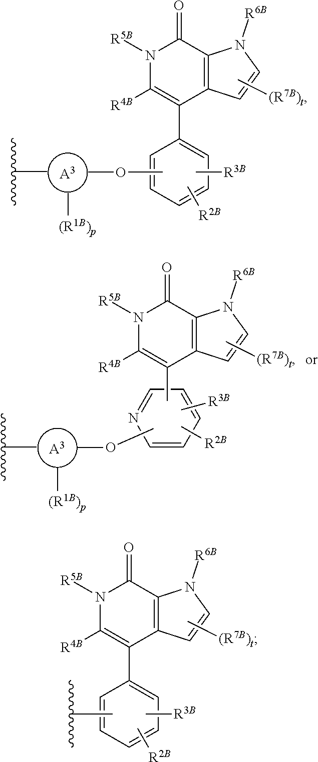
A³ is phenylene, pyridinylene, pyrazinylene, a 3-10 membered saturated monocyclic, bicyclic or spirocyclic carbocycylene, or C₁₋₆ alkylene;
R^{1B} represents independently for each occurrence halo, C₁₋₄ alkyl, or C₁₋₄ haloalkyl;
R^{2B} is hydrogen, halo, C₁₋₄ alkyl, or C₁₋₄ haloalkyl;
R^{3B} is —N(R^{8B})SO₂R^{9B}, —SO₂N(R^{8B})₂, —SO₂R^{9B}, —(C₁₋₆ alkylene)-SO₂R^{9B}, —(C₀₋₆ alkylene)-N(R^{8B})C(O)R^{9B}, —(C₀₋₆ alkylene)-C(O)N(R^{9B})₂, —N(R^{8B})₂, —NO₂, C₁₋₆ hydroxyalkyl, hydrogen, or a 4-7 membered saturated carbocyclic ring in which one CH₂ is replaced with SO₂;
R^{4B} is hydrogen, halo, or C₁₋₄ alkyl;
R^{8B} is C₁₋₄ alkyl or C₃₋₄ cycloalkyl;
R^{6B} is hydrogen, C₁₋₄ alkyl, or C₃₋₄ cycloalkyl;
R^{7B} represents independently for each occurrence C₁₋₄ alkyl or C₃₋₄ cycloalkyl;
R^{8B} represents independently for each occurrence hydrogen or C₁₋₄ alkyl; or two occurrences of R^{8B} are taken together with the nitrogen atom to which they are attached to form a 3-7 membered heterocyclic ring containing 1 or 2 heteroatoms independently selected from nitrogen and oxygen; or R^{8B} and R^{9B} are taken together with their intervening atoms to a form a 5-7 membered ring containing 1, 2, or 3 heteroatoms independently selected from nitrogen, oxygen, and sulfur;
R^{9B} is C₁₋₆ alkyl, C₁₋₆ haloalkyl, —(C₁₋₆ alkylene)-(C₃₋₆ cycloalkyl), or C₃₋₆ cycloalkyl;
L is a linker;
k is 1, 2, 3, or 4; and
m, n, p and t are independently 0, 1, or 2.

2. The compound of claim 1, wherein R² is methyl.

3. The compound of claim 1 or 2, wherein R³ is hydrogen.

4. The compound of any one of claims 1-3, wherein m is 0.

5. The compound of any one of claims 1-3, wherein R¹ is

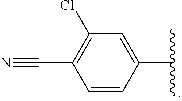 [SMILES: *c1ccc(C#N)c(Cl)c1]

6. The compound of any one of claims 1-5, wherein k is 4.

7. The compound of any one of claims 1-6, wherein the compound is a compound of Formula I.

8. A compound represented by Formula I-1*: 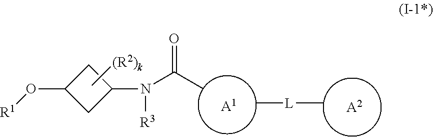
or a pharmaceutically acceptable salt thereof; wherein:
R¹ is phenyl substituted by cyano, halogen, and m occurrences of R⁴;
R² represents independently for each occurrence C₁₋₄ alkyl;
R³ is hydrogen or C₁₋₄ alkyl;
R⁴ is C₁₋₄ alkyl;
R⁵ represents independently for each occurrence C₁₋₄ alkyl or halogen, or one occurrence of R⁵ is taken together with R³ to form a C₁₋₃ alkylene;
A¹ is a pyridazinylene, pyrimidinylene, pyrazinylene, pyridinylene, phenylene, or C₅₋₆ cycloalkylene, each of which is substituted with n occurrences of R⁵;
A² is 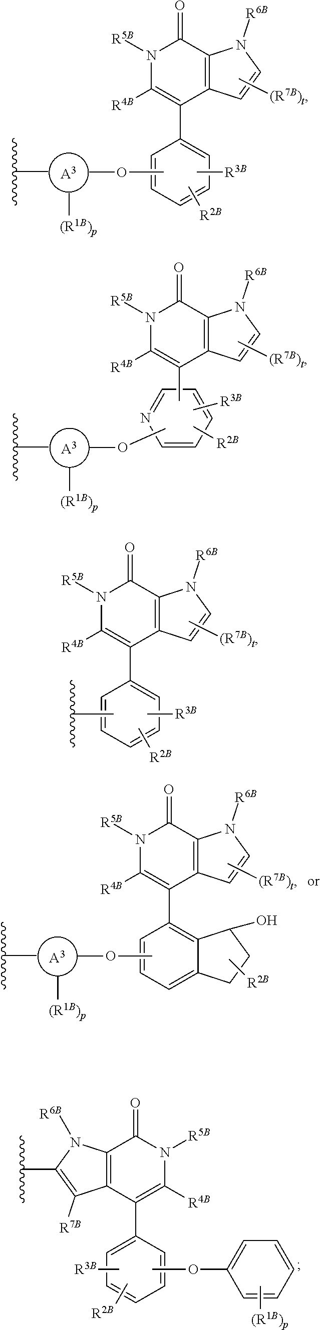
A³ is phenylene, pyridinylene, pyrazinylene, a 3-10 membered saturated monocyclic, bicyclic or spirocyclic carbocycylene, or C₁₋₆ alkylene;
R^{1B} represents independently for each occurrence halo, C₁₋₄ alkyl, or C₁₋₄ haloalkyl;
R^{2B} is hydrogen, halo, C₁₋₄ alkyl, or C₁₋₄ haloalkyl;
R^{3B} is —N(R^{8B})SO₂R^{9B}, —SO₂N(R^{8B})₂, —SO₂R^{9B}, —(C₁₋₆ alkylene)-SO₂R^{9B}, —(C₀₋₆ alkylene)-N(R^{8B})C(O)R^{9B}, —(C₀₋₆ alkylene)-C(O)N(R^{8B})₂, —N(R^{8B})₂, —NO₂, C₁₋₆ hydroxyalkyl, hydrogen, or a 4-7 membered saturated carbocyclic ring in which one CH₂ is replaced with SO₂;
R^{4B} is hydrogen, halo, or C₁₋₄ alkyl;
R^{5B} is C₁₋₄ alkyl or C₃₋₄ cycloalkyl;
R^{3B} is hydrogen, C₁₋₄ alkyl, or C₃₋₄ cycloalkyl;
R^{7B} represents independently for each occurrence C₁₋₄ alkyl or C₃₋₄ cycloalkyl;
R^{8B} represents independently for each occurrence hydrogen or C₁₋₄ alkyl; or two occurrences of R^{8B} are taken together with the nitrogen atom to which they are attached to form a 3-7 membered heterocyclic ring containing 1 or 2 heteroatoms independently selected from nitrogen and oxygen; or R^{8B} and R^{9B} are taken together with their intervening atoms to a form a 5-7 membered ring containing 1, 2, or 3 heteroatoms independently selected from nitrogen, oxygen, and sulfur;
R^{9B} is C₁₋₆ alkyl, C₁₋₆ haloalkyl, —(C₁₋₆ alkylene)-(C₃₋₆ cycloalkyl), or C₃₋₆ cycloalkyl;
L is a linker;
k is 1, 2, 3, or 4; and
m, n, p and t are independently 0, 1, or 2.

9. The compound of claim 1, 2, or 8, wherein the compound is a compound of Formula Ia or a pharmaceutically acceptable salt thereof:

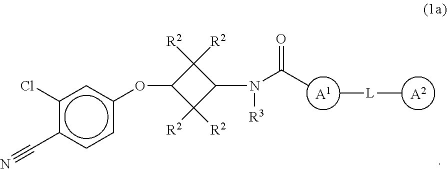

10. The compound of claim 1, 2, or 8, wherein the compound is a compound of Formula Ib or a pharmaceutically acceptable salt thereof:

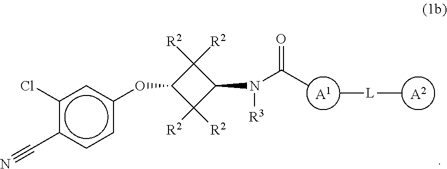

11. The compound of claim 1 or 8, wherein the compound is a compound of Formula Ic or a pharmaceutically acceptable salt thereof:

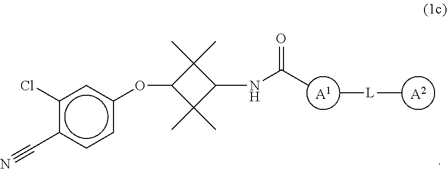

12. The compound of claim 1 or 8, wherein the compound is a compound of Formula Id or a pharmaceutically acceptable salt thereof:

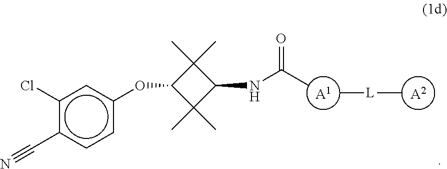

13. The compound of any one of claims 1-12, wherein A¹ is pyridazinylene substituted with n occurrences of R⁵.

14. The compound of any one of claims 1-12, wherein A¹ is

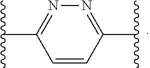 [SMILES: *c1ccc(*)nn1]

15. The compound of any one of claims 1-12, wherein A¹ is pyrimidinylene substituted with n occurrences of R⁵.

16. The compound of any one of claims 1-12, wherein A¹ is 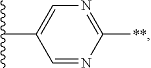
where ** is point of attachment to L.

17. The compound of any one of claims 1-12, wherein A¹ is 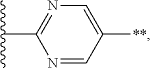
where ** is point of attachment to L.

18. The compound of any one of claims 1-12, wherein A¹ is pyrazinylene substituted with n occurrences of R⁵.

19. The compound of any one of claims 1-12, wherein A¹

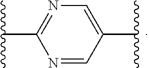 [SMILES: *c1cnc(*)nc1]

20. The compound of any one of claims 1-12, wherein A¹ is pyridinylene substituted with n occurrences of R⁵.

21. The compound of any one of claims 1-12, wherein A¹ is 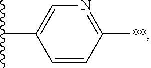
where ** is point of attachment to L.

22. The compound of any one of claims 1-10, wherein A¹ is phenylene substituted with n occurrences of R⁵.

23. The compound of any one of claims 1-10, wherein A¹ is

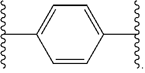 [SMILES: *c1ccc(*)cc1]

24. The compound of any one of claims 1-13, 15, 18, or 20, wherein n is 0.

25. The compound of any one of claims 1-24, wherein A² is

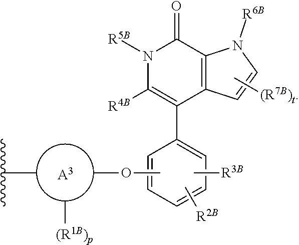

26. The compound of any one of claims 1-24, wherein A² is

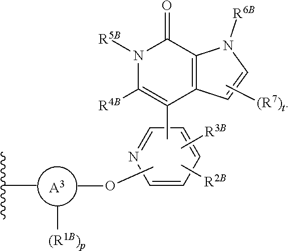

27. The compound of any one of claims 1-26, wherein A³ is a 3-10 membered saturated monocyclic, bicyclic or spirocyclic carbocycylene.

28. The compound of any one of claims 1-24, wherein A² is

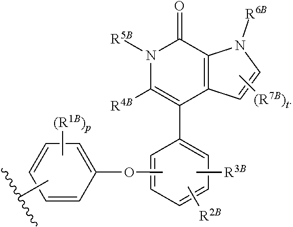

29. The compound of any one of claims 1-24, wherein A² is

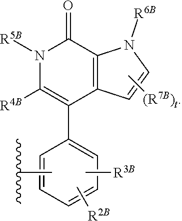

30. The compound of any one of claims 1-24, wherein A² is

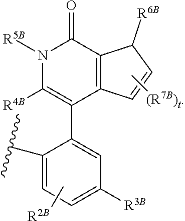

31. The compound of any one of claims 1-30, wherein R^{2B} is hydrogen.

32. The compound of any one of claims 1-31, wherein R^{3B} is —N(R^{8B})SO₂R^{9B}.

33. The compound of any one of claims 1-31, wherein R^{3B} is —SO₂R^{9B}.

34. The compound of any one of claims 1-31, wherein R^{3B} is —(C₁₋₆ alkylene)-SO₂R^{9B}.

35. The compound of any one of claims 1-31, wherein R^{3B} is —SO₂N(R^{8B})₂.

36. The compound of any one of claims 1-31, wherein R^{3B} is —N(R^{8B})C(O)R^{9B}.

37. The compound of any one of claims 1-31, wherein R^{3B} is —N(R^{8B})₂.

38. The compound of any one of claims 1-31, wherein R^{3B} is C₁₋₆ hydroxyalkyl.

39. The compound of any one of claims 1-34 or 36, wherein R^{9B} is C₁₋₆ alkyl or C₁₋₆ haloalkyl.

40. The compound of any one of claims 1-39, wherein R^{4B} is hydrogen.

41. The compound of any one of claims 1-40, wherein R^{5B} is C₁₋₄ alkyl.

42. The compound of any one of claims 1-40, wherein R^{5B} is methyl.

43. The compound of any one of claims 1-42, wherein R^{6B} is hydrogen.

44. The compound of any one of claims 1-43, wherein R^{7B} is C₁₋₄ alkyl.

45. The compound of any one of claims 1-43, wherein R^{7B} is methyl.

46. The compound of any one of claims 1-45, wherein t is 2.

47. The compound of any one of claims 1-24, wherein A² is

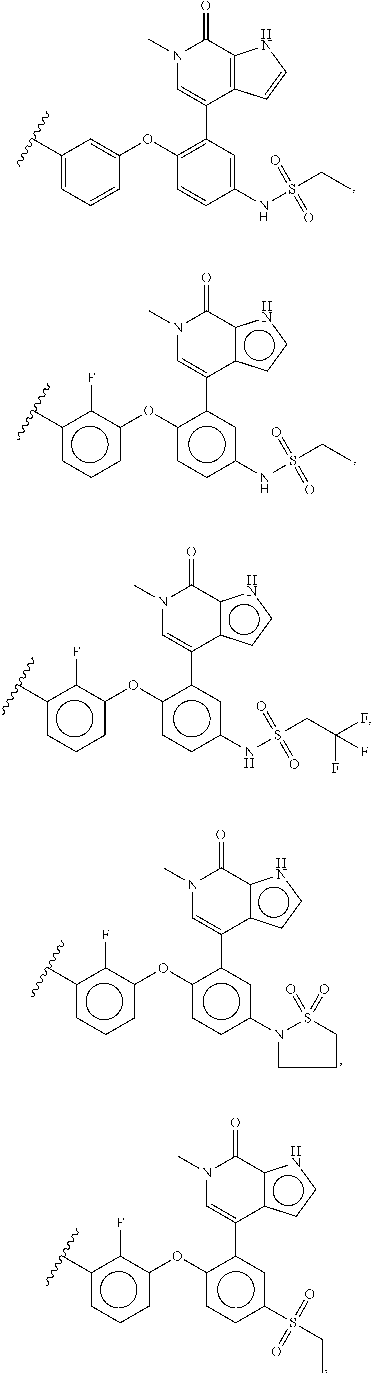

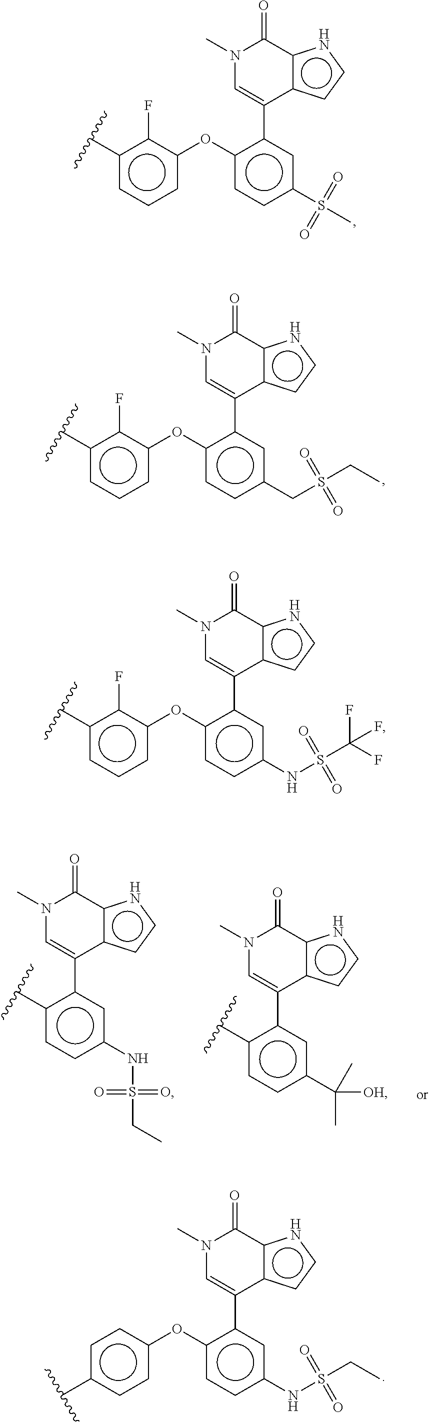

48. The compound of any one of claims 1-24, wherein A² is

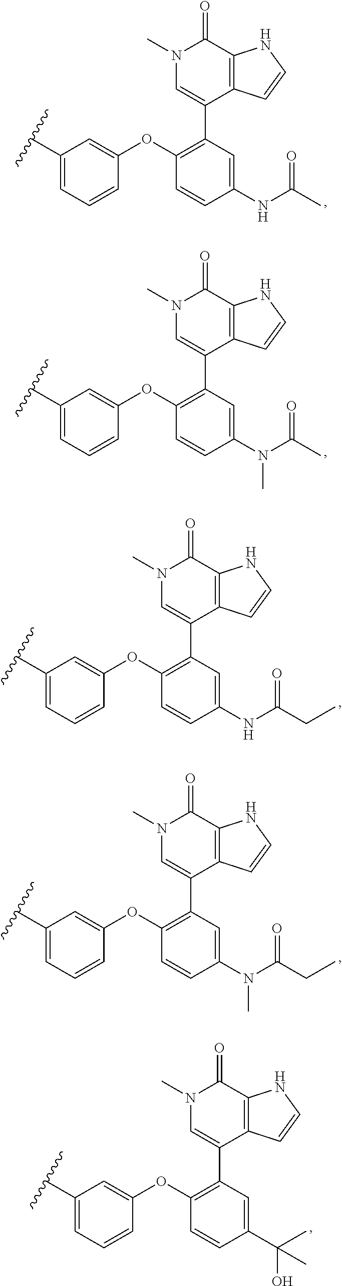

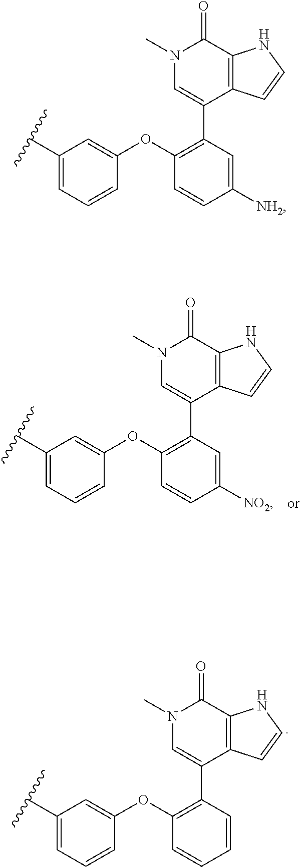 [SMILES: *c1cccc(Oc2ccc(N)cc2-c2cn(C)c(=O)c3[nH]ccc23)c1 ; *c1cccc(Oc2ccc([N+](=O)[O-])cc2-c2cn(C)c(=O)c3[nH]ccc23)c1 ; *c1cccc(Oc2ccccc2-c2cn(C)c(=O)c3[nH]ccc23)c1]

49. The compound of any one of claims 1-24, wherein A² is

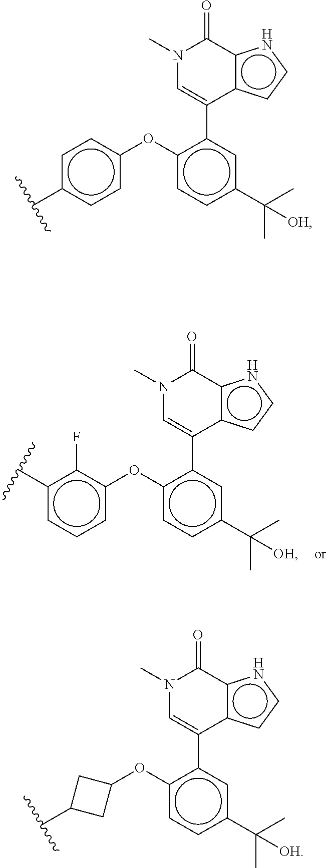

50. The compound of claim 1, wherein the compound is represented by Formula Ie: 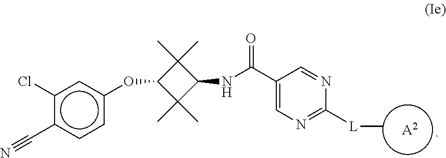
or a pharmaceutically acceptable salt thereof; wherein
A² is 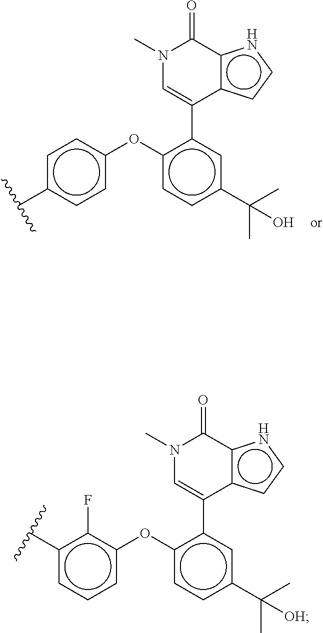
and
L is a linker.

51. The compound of any one of claims 1-50, wherein L is a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —C(O)—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, —N(C₃₋₆ cycloalkyl)-, —C(H)(C₃₋₆ cycloalkyl)-, optionally substituted 3-10 membered carbocyclyl, or optionally substituted 3-11 membered heterocyclyl containing 1, 2, 3, or 4 heteroatoms independently selected from nitrogen, oxygen, and sulfur.

52. The compound of any one of claims 1-50, wherein L is a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C i-₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, or optionally substituted 3-10 membered heterocyclyl containing 1, 2, 3, or 4 heteroatoms independently selected from nitrogen, oxygen, and sulfur.

53. The compound of any one of claims 1-50, wherein L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —C(O)—, —N(H)C(O)—, —N(C₁₋₆ alkyl)C(O)—, —C(O)N(H)—, —C(O)N(C₁₋₆ alkyl)-, —N(C₃₋₆ cycloalkyl)-, —C(H)(C₃₋₆ cycloalkyl)-, 3-10 membered carbocyclyl, or 3-11 membered heterocyclyl containing 1, 2, or 3 heteroatoms independently selected from nitrogen, oxygen, and sulfur.

54. The compound of any one of claims 1-50, wherein L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —N(H)C(O)—, —N(C₁₋₆ alkyl)C(O)—, —C(O)N(H)—, —C(O)N(C₁₋₆ alkyl)-, 3-10 membered carbocyclyl, or 3-10 membered heterocyclyl containing 1, 2, or 3 heteroatoms independently selected from nitrogen, oxygen, and sulfur.

55. The compound of any one of claims 1-50, wherein L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —C(O)—, —N(H)C(O)—, —N(C₁₋₆ alkyl)C(O)—, —C(O)N(H)—, —C(O)N(C₁₋₆ alkyl)-, —N(C₃₋₆ cycloalkyl)-, or —C(H)(C₃₋₆ cycloalkyl)-.

56. The compound of any one of claims 1-50, wherein L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —N(H)C(O)—, —N(C₁₋₆ alkyl)C(O)—, —C(O)N(H)—, or —C(O)N(C₁₋₆ alkyl)-.

57. The compound of any one of claims 1-50, wherein L is a bivalent, saturated or unsaturated, straight or branched C₅₋₄₀ hydrocarbon chain, wherein 1-20 methylene units of the hydrocarbon are independently replaced with —O—, —N(H)—, —N(C₁₋₆ alkyl)-, —N(H)C(O)—, —N(C₁₋₆ alkyl)C(O)—, —C(O)N(H)—, —C(O)N(C₁₋₆ alkyl)-, optionally substituted 3-10 membered carbocyclyl, or optionally substituted 3-10 membered heterocyclyl containing 1, 2, 3, or 4 heteroatoms independently selected from nitrogen, oxygen, and sulfur.

58. The compound of any one of claims 1-50, wherein L is -(3-7 membered, monocyclic, saturated heterocyclic ring containing 1 or 2 heteroatoms selected from nitrogen)-(OCH₂CH₂)₁₋₁₅—O—***, wherein *** is the point of attachment to A².

59. The compound of any one of claims 1-50, wherein L is -(3-7 membered, monocyclic, saturated heterocyclic ring containing 1 or 2 heteroatoms selected from nitrogen)-(OCH₂CH₂)₁₋₅—O—***, wherein *** is the point of attachment to A².

60. The compound of any one of claims 1-50, wherein L is -(3-7 membered, monocyclic, saturated heterocyclic ring containing 1 or 2 heteroatoms selected from nitrogen)-(OCH₂CH₂)₆₋₁₀—O—***, wherein *** is the point of attachment to A².

61. The compound of any one of claims 1-50, wherein L is -piperidinylene-(OCH₂CH₂)₁₋₁₅—O—***, wherein *** is the point of attachment to A².

62. The compound of any one of claims 1-50, wherein L is 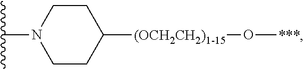
wherein *** is the point of attachment to A².

63. The compound of any one of claims 1-50, wherein L is 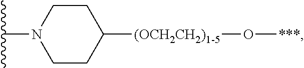
wherein *** is the point of attachment to A².

64. The compound of any one of claims 1-50, wherein L is 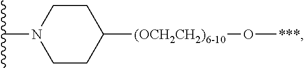
wherein *** is the point of attachment to A².

65. The compound of any one of claims 1-50, wherein L is -(3-7 membered, monocyclic, saturated heterocyclic ring containing 1 or 2 heteroatoms selected from nitrogen)-(OCH₂CH₂)₁₋₁₅—N(H)C(O)—C₁₋₁₀ alkylene-***, -(3-7 membered, monocyclic, saturated heterocyclic ring containing 1 or 2 heteroatoms selected from nitrogen)-(OCH₂CH₂)₁₋₁₅—N(C₁₋₄ alkyl)C(O)—C₁₋₁₀ alkylene-***, -(3-7 membered, monocyclic, saturated heterocyclic ring containing 1 or 2 heteroatoms selected from nitrogen)-(OCH₂CH₂)₁₋₁₅—C(O)N(H)—C₁-10 alkylene-***, or -(3-7 membered, monocyclic, saturated heterocyclic ring containing 1 or 2 heteroatoms selected from nitrogen)-(OCH₂CH₂)₁₋₁₅—C(O)N(C₁₋₄ alkyl)-C₁₋₁₀ alkylene-***, wherein *** is the point of attachment to A².

66. The compound of any one of claims 1-50, wherein L is -piperidinylene-(OCH₂CH₂)₁₋₅—N(H)C(O)—C₁₋₅ alkylene-***, -piperidinylene-(OCH₂CH₂)₁₋₅—N(C₁₋₄ alkyl)C(O)—C₁₋₅ alkylene-***, -piperidinylene-(OCH₂CH₂)₁₋₅—C(O)N(H)—C₁₋₅ alkylene-***, or -piperidinylene-(OCH₂CH₂)₁₋₅—C(O)N(C₁₋₄ alkyl)-C₁₋₅ alkylene-***, wherein *** is the point of attachment to A².

67. The compound of any one of claims 1-50, wherein L is -(3-7 membered, monocyclic, saturated heterocyclic ring containing 1 or 2 heteroatoms selected from nitrogen)-(OCH₂CH₂)₁₋₁₀—***, -(3-7 membered, monocyclic, saturated heterocyclic ring containing 1 or 2 heteroatoms selected from nitrogen)-(C₀₋₁₀ alkylene)-O—***, or -(3-7 membered, monocyclic, saturated heterocyclic ring containing 1 or 2 heteroatoms selected from nitrogen)-C₁₋₁₀ alkylene, wherein *** is the point of attachment to A².

68. The compound of any one of claims 1-50, wherein L is -piperidinylene-(OCH₂CH₂)₁₋₅—***, -piperidinylene-(C₀₋₅ alkylene)-O—***, or -piperidinylene-(C₁₋₅ alkylene)-***, wherein *** is the point of attachment to A².

69. The compound of any one of claims 1-50, wherein L is -(3-7 membered monocyclic saturated heterocyclic ring containing 1 or 2 heteroatoms selected from nitrogen)-X¹—***, wherein *** is the point of attachment to A², and X¹ is (i) C₁₋₁₀ alkylene where 1 or 2 methylene groups are optionally replaced by —O—, —N(H)—, or —N(C₁₋₄ alkyl)-, (ii) a 3-7 membered monocyclic saturated heterocyclic ring containing 1 or 2 heteroatoms selected from nitrogen, or (iii) -(3-7 membered monocyclic saturated heterocyclic ring containing 1 or 2 heteroatoms selected from nitrogen)-(C₁₋₁₀ alkylene)-.

70. The compound of any one of claims 1-50, wherein L is -(piperidinylene)-X¹—***, wherein *** is the point of attachment to A², and X¹ is (i) C₁₋₅ alkylene where 1 or 2 methylene groups are optionally replaced by —O—, —N(H)—, or —N(C₁₋₄ alkyl)-, (ii) a 3-4 membered monocyclic saturated heterocyclic ring containing 1 heteroatom selected from nitrogen, or (iii) -(3-4 membered monocyclic saturated heterocyclic ring containing 1 heteroatom selected from nitrogen)-(C₁₋₅ alkylene)-.

71. The compound of any one of claims 1-50, wherein L is 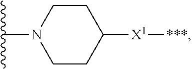
wherein *** is the point of attachment to A², and X¹ is (i) C₁₋₁₀ alkylene where 1 or 2 methylene groups are optionally replaced by —O—, —N(H)—, or —N(C₁₋₄ alkyl)-, (ii) a 3-7 membered monocyclic saturated heterocyclic ring containing 1 or 2 heteroatoms selected from nitrogen, or (iii) -(3-7 membered monocyclic saturated heterocyclic ring containing 1 or 2 heteroatoms selected from nitrogen)-(C₁₋₁₀ alkylene)-.

72. The compound of any one of claims 1-50, wherein L is -(piperazinylene)-X¹—***, wherein *** is the point of attachment to A², and X¹ is (i) C₁₋₅ alkylene where 1 or 2 methylene groups are optionally replaced by —O—, (ii) a 3-4 membered monocyclic saturated heterocyclic ring containing 1 heteroatom selected from nitrogen, or (iii) -(3-4 membered monocyclic saturated heterocyclic ring containing 1 heteroatom selected from nitrogen)-(C₁₋₅ alkylene)-.

73. The compound of any one of claims 1-50, wherein L is 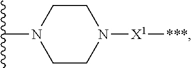
wherein *** is the point of attachment to A², and X¹ is (i) C₁₋₁₀ alkylene where 1 or 2 methylene groups are optionally replaced by —O—, (ii) a 3-7 membered monocyclic saturated heterocyclic ring containing 1 or 2 heteroatoms selected from nitrogen, or (iii) -(3-7 membered monocyclic saturated heterocyclic ring containing 1 or 2 heteroatoms selected from nitrogen)-(C₁₋₁₀ alkylene)-.

74. The compound of any one of claims 1-50, wherein L is -(3-7 membered monocyclic saturated heterocyclic ring containing 1 or 2 heteroatoms selected from nitrogen)-X²-(C₁₋₁₀ alkylene)-***, wherein *** is the point of attachment to A², and X² is —O—, —N(H)—, or —N(C₁₋₆ alkyl)-.

75. The compound of any one of claims 1-50, wherein L is -(piperidinylene)-X²—(C₁₋₁₀ alkylene)-***, wherein *** is the point of attachment to A², and X² is —O—, —N(H)—, or —N(C₁₋₆ alkyl)-.

76. The compound of any one of claims 1-50, wherein L is -(piperidinylene)-X²-(a 3-7 membered monocyclic saturated heterocyclic ring containing 1 or 2 heteroatoms selected from nitrogen)-***, wherein *** is the point of attachment to A², and X² is —O—, —N(H)—, or —N(C₁₋₆ alkyl)-.

77. The compound of any one of claims 1-50, wherein L is 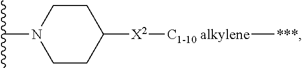
wherein *** is the point of attachment to A², and X² is —O—, —N(H)—, or —N(C₁₋₆ alkyl)-.

78. The compound of any one of claims 1-50, wherein L is 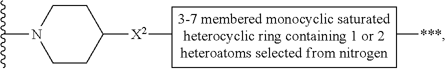
wherein *** is the point of attachment to A², and X² is —O—, —N(H)—, or —N(C₁₋₆ alkyl)-.

79. The compound of any one of claims 74-78, wherein X² is —O—.

80. The compound of any one of claims 1-50, wherein L is -(3-7 membered monocyclic saturated heterocyclic ring containing 1 or 2 heteroatoms selected from nitrogen)-X¹—***, wherein *** is the point of attachment to A², and X¹ is —(OCH₂CH₂)₁₋₁₀ where 1 CH₂ group is optionally replaced with —C(H)(C₃₋₆ cycloalkyl)-.

81. The compound of any one of claims 1-50, wherein L is a 7-11 membered spirocyclic or fused bicyclic saturated heterocyclic ring containing 1, 2, or 3 heteroatoms selected from nitrogen and oxygen.

82. The compound of any one of claims 1-50, wherein L is a 7-8 membered spirocyclic or fused bicyclic saturated heterocyclic ring containing 2 heteroatoms selected from nitrogen.

83. The compound of any one of claims 1-50, wherein L is -(3-7 membered monocyclic saturated heterocyclic ring containing 1 or 2 heteroatoms selected from nitrogen)-X³-(3-7 membered monocyclic saturated heterocyclic ring containing 1 or 2 heteroatoms selected from nitrogen)-, wherein X³ is C₁₋₁₀ alkylene, —O—, —N(H)—, —N(C₁₋₄ alkyl)-, or a bond.

84. The compound of any one of claims 1-50, wherein L is -(piperidinylene)-(C₁₋₅ alkylene)-(piperazinylene)-***, wherein *** is the point of attachment to A².

85. The compound of any one of claims 1-50, wherein L is -(piperazinylene)-(azetidinylene)-*** or (azetidinylene)-(piperazinylene)-***, wherein *** is the point of attachment to A².

86. The compound of any one of claims 1-50, wherein L is -(piperidinylene)-X³-(azetidinylene)- or -(azetidinylene)-X³-(azetidinylene)-, wherein X³ is C₁₋₃ alkylene, and *** is the point of attachment to A².

87. The compound of any one of claims 1-50, wherein L is -(3-7 membered monocyclic saturated heterocyclic ring containing 1 or 2 heteroatoms selected from nitrogen)-X³—(C₃₋₆ cycloalkylene)-O—***, -(3-7 membered monocyclic saturated heterocyclic ring containing 1 or 2 heteroatoms selected from nitrogen)-X³—(C₃₋₆ cycloalkylene)-N(H)—***, or -(3-7 membered monocyclic saturated heterocyclic ring containing 1 or 2 heteroatoms selected from nitrogen)-X³—(C₃₋₆ cycloalkylene)-N(C₁₋₄ alkyl)-***, wherein *** is the point of attachment to A², and X³ is C₁₋₁₀ alkylene, —O—, —N(H)—, —N(C₁₋₄ alkyl)-, or a bond.

88. The compound of any one of claims 1-50, wherein L is -(piperidinylene)-X³—(C₃₋₆ cycloalkylene)-O—***, -(piperidinylene)-X³—(C₃₋₆ cycloalkylene)-N(H)—***, or -(piperidinylene)-X³—(C₃₋₆ cycloalkylene)-***, wherein *** is the point of attachment to A², and X³ is C₁₋₁₀ alkylene, —O—, —N(H)—, —N(C₁₋₄ alkyl)-, or a bond.

89. The compound of any one of claims 1-50, wherein L is —N(C₁₋₃ alkyl)-(C₂₋₇ alkylene)-O—***, —N(H)—(C₂₋₇ alkylene)-O—***, —N(C₁₋₃ alkyl)-[(C₂₋₄ alkylene)-O—]₂₋₁₀—***, or —N(H)—[(C₂₋₄ alkylene)-O—]₂₋₁₀—***, where *** is a point of attachment to A².

90. The compound of any one of claims 1-50, wherein L is —N(C₁₋₃ alkyl)-(C₁₋₆ alkylene)-(5-6 membered saturated heterocyclylene containing 1 or 2 heteratoms independently selected from nitrogen and oxygen)-(C₁₋₆ alkylene)-O—*** or —N(H)—(C₁₋₆ alkylene)-(5-6 membered saturated heterocyclylene containing 1 or 2 heteratoms independently selected from nitrogen and oxygen)-(C₁₋₆ alkylene)-O—***, where *** is a point of attachment to A².

91. The compound of any one of claims 1-50, wherein L has the formula —(C₀₋₁₂ alkylene)-(optionally substituted 3-40 membered heteroalkylene)-(C₀₋₁₂ alkylene)-.

92. The compound of any one of claims 1-50, wherein L is one of the following: 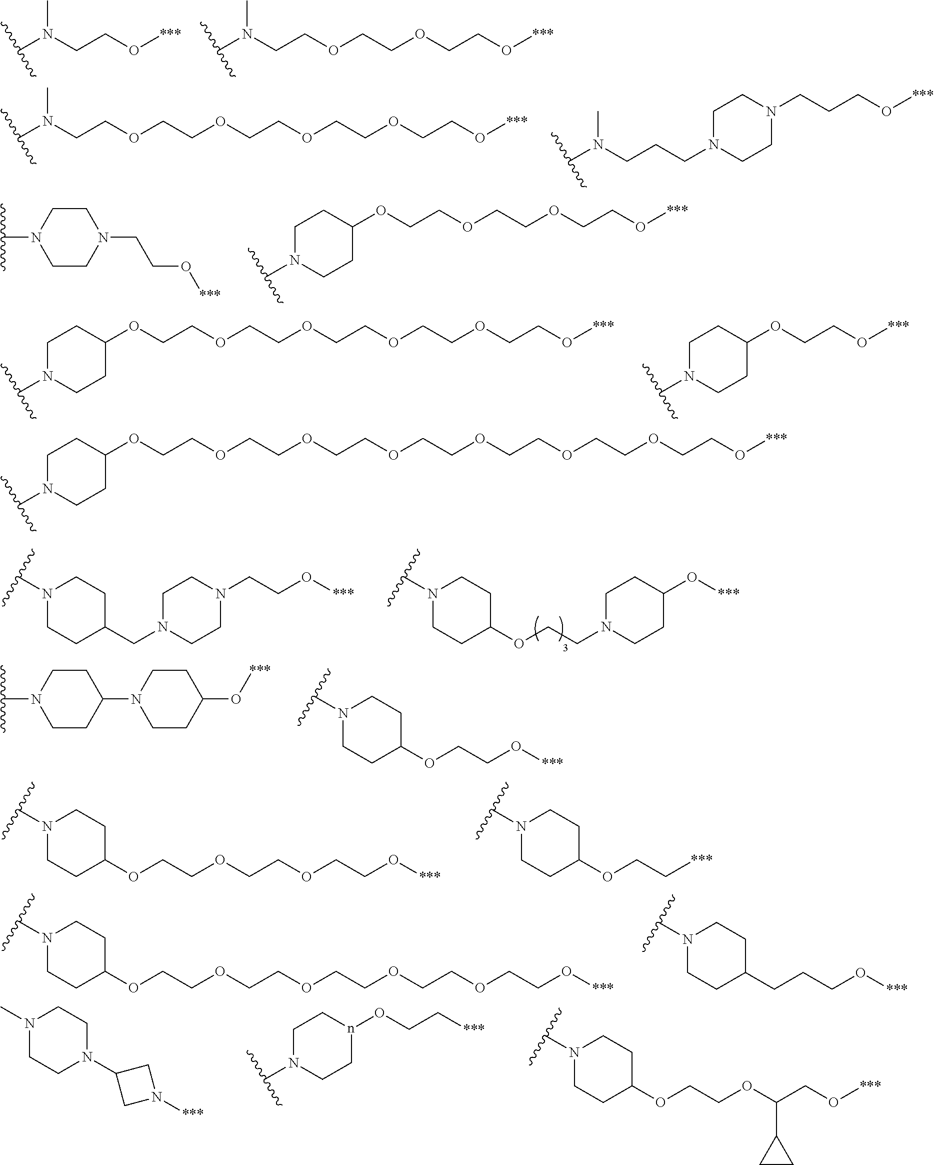
wherein *** is the point of attachment to A².

93. The compound of any one of claims 1-50, wherein L is one of the following: 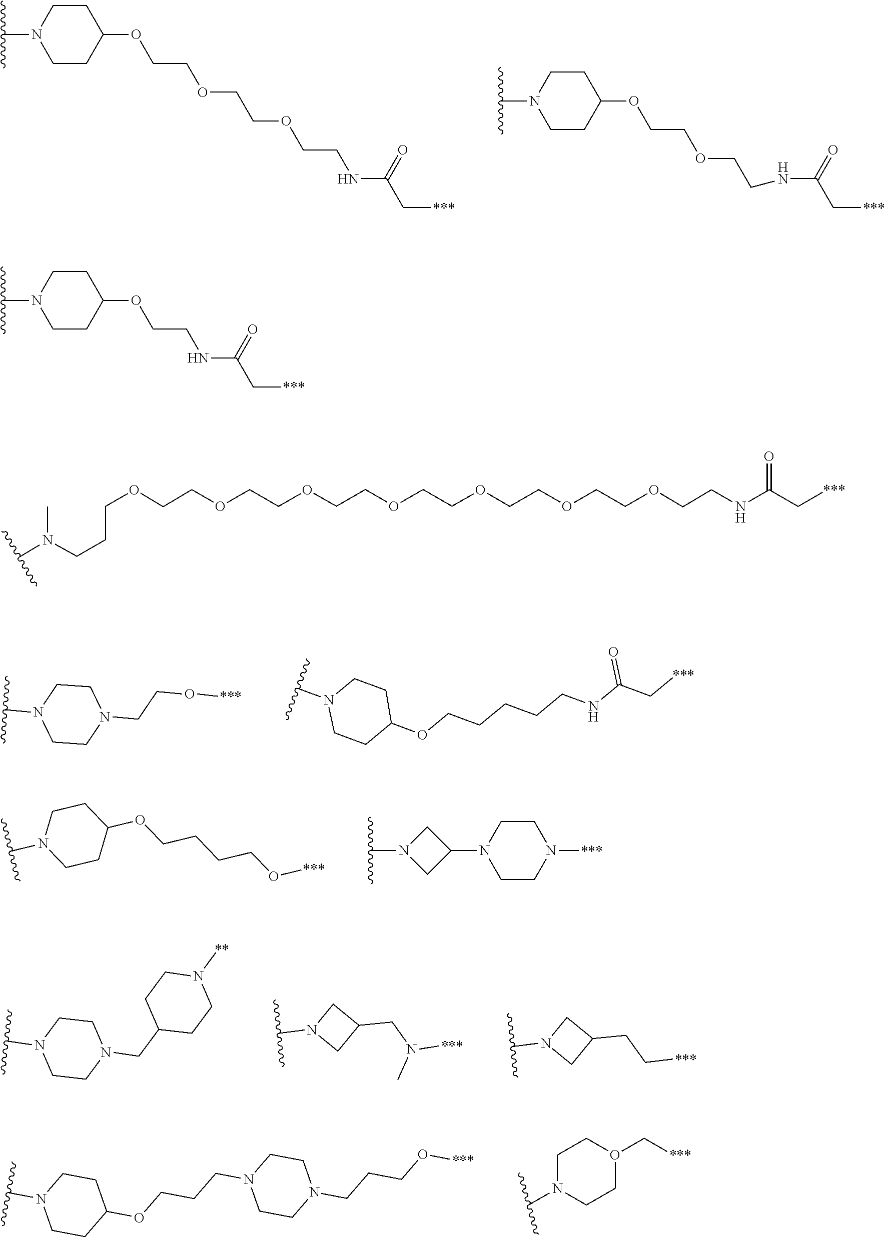 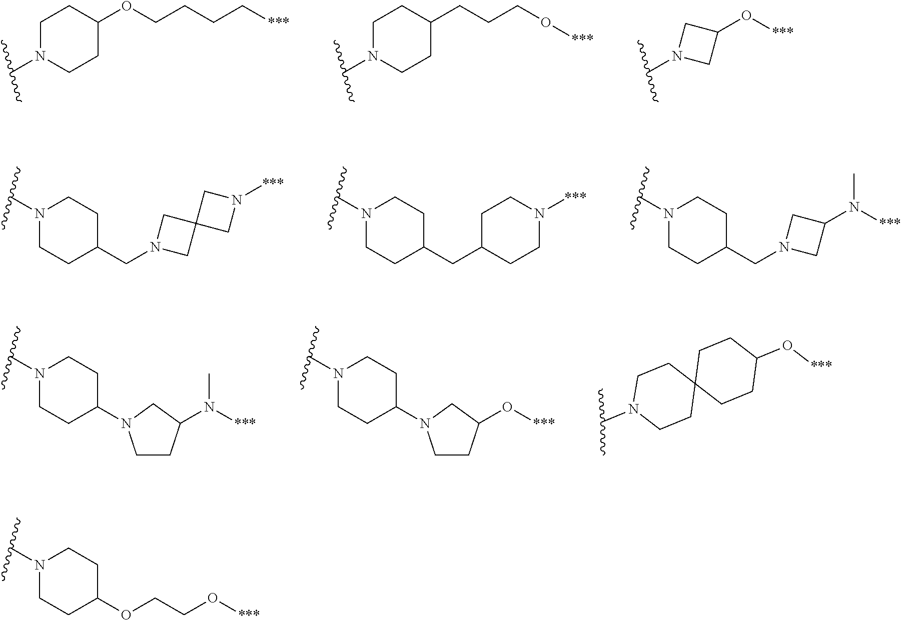
wherein *** is the point of attachment to A².

94. The compound of any one of claims 1-50, wherein L is C₄₋₁₄ alkylene.

95. The compound of any one of claims 1-50, wherein L is one of the following: 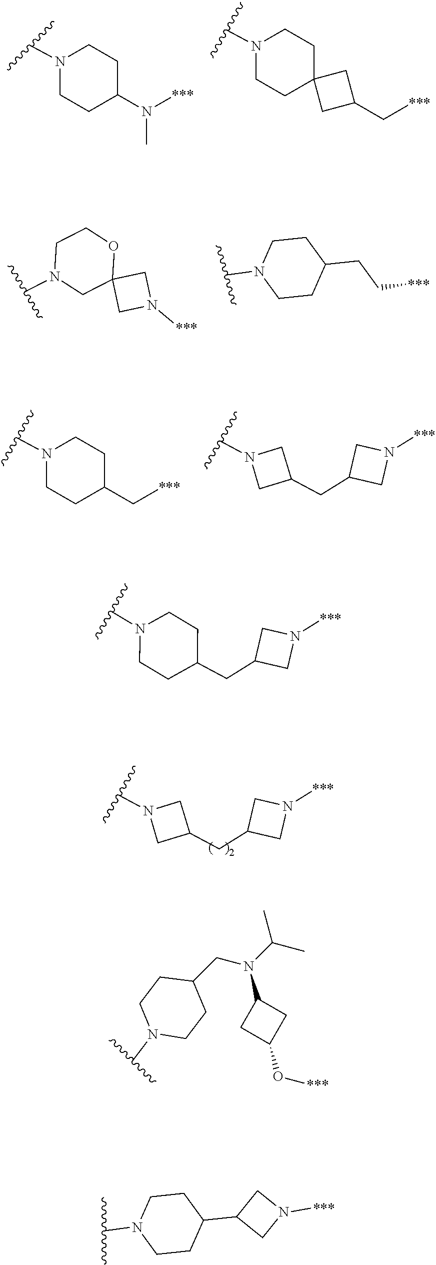
wherein *** is the point of attachment to A².

96. The compound of claim 1, wherein the compound is represented by Formula Ig: 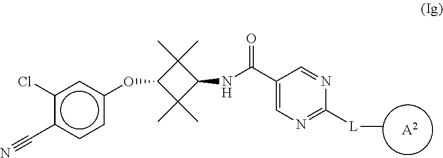
or a pharmaceutically acceptable salt thereof; wherein
A² is 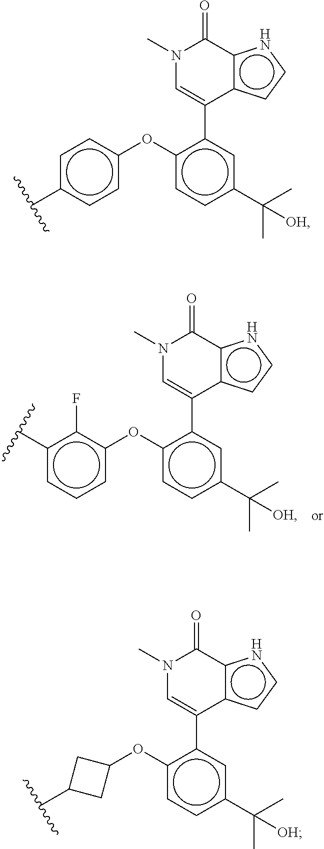
and
L is one of the following:
-(3-7 membered, monocyclic, saturated heterocyclic ring containing 1 or 2 heteroatoms selected from nitrogen)-C₁₋₁₀ alkylene, wherein *** is the point of attachment to A²;
-(piperidinylene)-X¹—***, wherein *** is the point of attachment to A², and X¹ is (i) C₁₋₅ alkylene where 1 or 2 methylene groups are optionally replaced by —O—, —N(H)—, or —N(C₁₋₄ alkyl)-;
-(7-11 membered spirocyclic or fused bicyclic saturated heterocyclic ring containing 1, 2, or 3 heteroatoms selected from nitrogen and oxygen)-(C₀₋₆ alkylene)-***, wherein *** is the point of attachment to A²;
-(3-7 membered monocyclic saturated heterocyclic ring containing 1 or 2 heteroatoms selected from nitrogen)-X³-(3-7 membered monocyclic saturated heterocyclic ring containing 1 or 2 heteroatoms selected from nitrogen)-, wherein X³ is C₁₋₁₀ alkylene, —O—, —N(H)—, —N(C₁₋₄ alkyl)-, or a bond; or
-(3-7 membered monocyclic saturated heterocyclic ring containing 1 or 2 heteroatoms selected from nitrogen)-X³—(C₃₋₅ cycloalkylene)-***, wherein *** is the point of attachment to A², and wherein X³ is C₁₋₁₀ alkylene, —O—, —N(H)—, —N(C₁₋₄ alkyl)-, or a bond.

97. The compound of claim 96, wherein L is -piperidinylene-(C₁₋₅ alkylene)-***, -(piperidinylene)-X³-(azetidinylene)-, or -(azetidinylene)-X³-(azetidinylene)-, wherein X³ is C₁₋₃ alkylene, and *** is the point of attachment to A².

98. A compound represented by Formula IL: 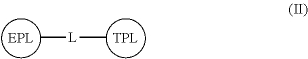
or a pharmaceutically acceptable salt thereof; wherein:
TPL is a group defined by Formula II-1 that is substituted by one occurrence of R^{III-1A}, wherein Formula II-1 is represented by: 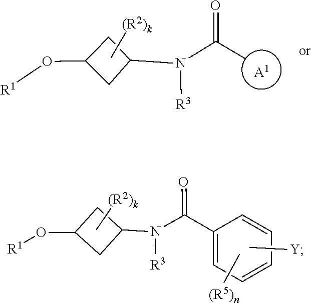
wherein:
R^{II-1A} is a bond to L;
R¹ is phenyl substituted by cyano, halogen, and m occurrences of R⁴;
R² and R³ each represent independently for each occurrence hydrogen or C₁₋₄ alkyl;
R⁴ is C₁₋₄ alkyl;
R⁵ represents independently for each occurrence C₁₋₄ alkyl or halogen;
A¹ is a pyridazinyl, pyrimidinyl, pyrazinyl, or pyridinyl, each of which is substituted with n occurrences of R⁵;
Y is —N(R²) or a 3-7 membered saturated heterocyclyl containing 1 or 2 heteroatoms selected from oxygen and nitrogen;
L is a linker;
EPL is a moiety that binds to BRD4;
k is 1, 2, 3, or 4; and
in and n are independently 0, 1, or 2.

99. The compound of claim 98, wherein the TPL is 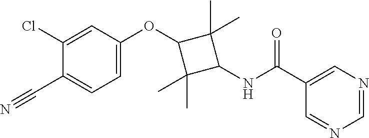 [SMILES: CC1(C)C(NC(=O)c2cncnc2)C(C)(C)C1Oc1ccc(C#N)c(Cl)c1]
that is substituted by one occurrence of R^{III-1A}.

100. The compound of claim 98, wherein the TPL is

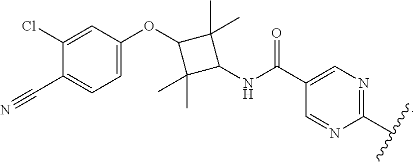 [SMILES: *c1ncc(C(=O)NC2C(C)(C)C(Oc3ccc(C#N)c(Cl)c3)C2(C)C)cn1]

101. The compound of any one of claims 98-100, wherein the EPL is defined by Formula II-2 that is substituted by one occurrence of R^{II-2A} wherein Formula III-2 is represented by: 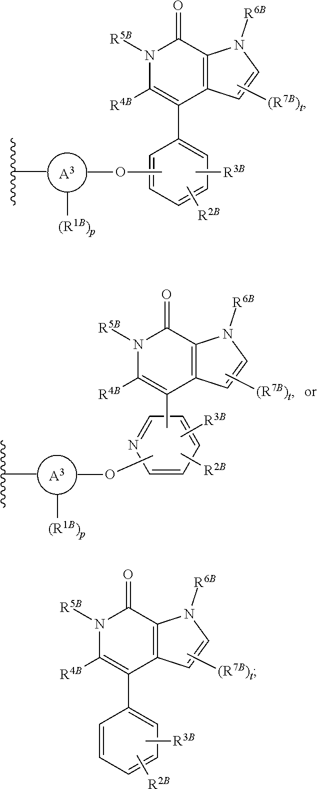
wherein
R^{II-2A} is a bond to L;
A³ is phenylene, a 3-10 membered saturated monocyclic, bicyclic or spirocyclic carbocycylene, or C₁₋₆ alkylene;
R^{1B} represents independently for each occurrence halo, C₁₋₄ alkyl, or C₁₋₄ haloalkyl;
R^{2B} is hydrogen, halo, C₁₋₄ alkyl, or C₁₋₄ haloalkyl;
R^{3B} is —N(R^{8B})SO₂R^{9B}, —SO₂N(R^{1B})₂, —SO₂R^{9B}, —(C₁₋₆ alkylene)-SO₂R^{9B}, —(C₀₋₆ alkylene)-N(R^{8B})C(O)R^{9B}, —(C₀₋₆ alkylene)-C(O)N(R^{8B})₂, —N(R^{8B})₂, —NO₂, C₁₋₆ hydroxyalkyl, hydrogen, or a 4-7 membered saturated carbocyclic ring in which one CH₂ is replaced with SO₂;
R^{4B} is hydrogen, halo, or C₁₋₄ alkyl;
R^{8B} is C₁₋₄ alkyl or C₃₋₄ cycloalkyl;
R^{6B} is hydrogen, C₁₋₄ alkyl, or C₃₋₄ cycloalkyl;
R^{7B} represents independently for each occurrence C₁₋₄ alkyl or C₃₋₄ cycloalkyl;
R^{8B} represents independently for each occurrence hydrogen or C₁₋₄ alkyl; or two occurrences of R^{8B} are taken together with the nitrogen atom to which they are attached to form a 3-7 membered heterocyclic ring containing 1 or 2 heteroatoms independently selected from nitrogen and oxygen; or R^{8B} and R^{9B} are taken together with their intervening atoms to a form a 5-7 membered ring containing 1, 2, or 3 heteroatoms independently selected from nitrogen, oxygen, and sulfur;
R^{9B} is C₁₋₆ alkyl, C₁₋₆ haloalkyl, —(C₁₋₆ alkylene)-(C₃₋₆ cycloalkyl), or C₃₋₆ cycloalkyl; and
p and t are independently 0, 1, or 2.

102. The compound of any one of claims 98-100, wherein the EPL is 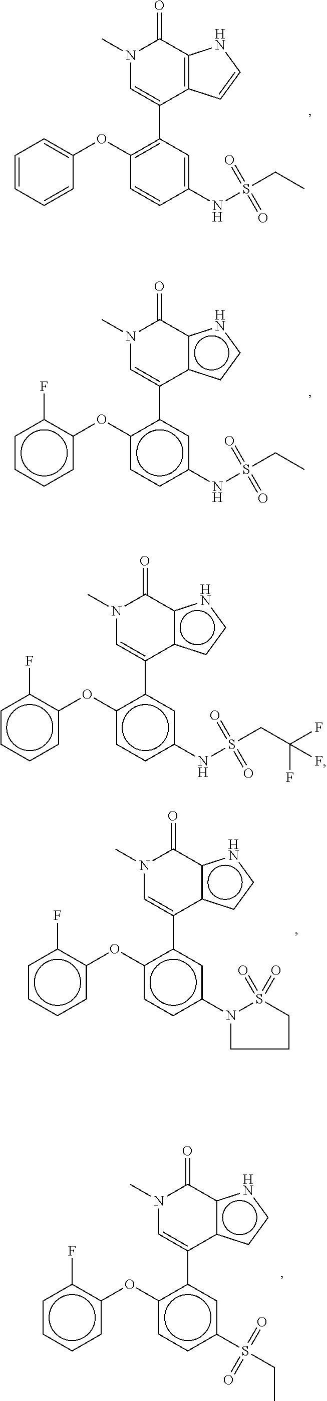 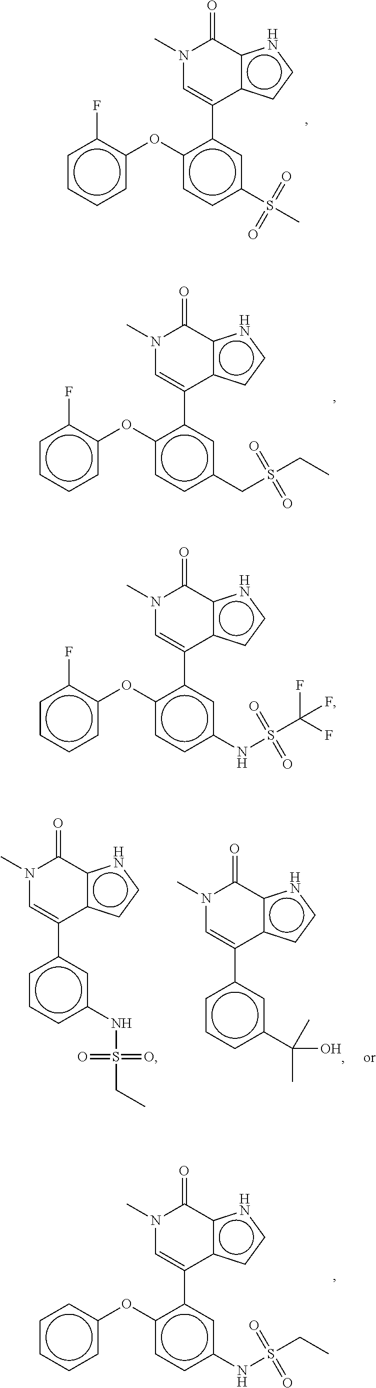
each of which is substituted by one occurrence of R^{II-2A}, wherein R^{II-2A} is a bond to L.

103. The compound of any one of claims 98-100, wherein the EPL is 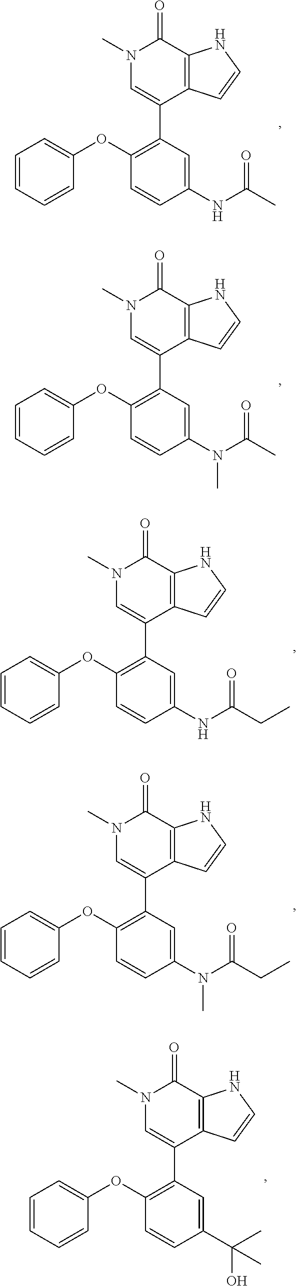 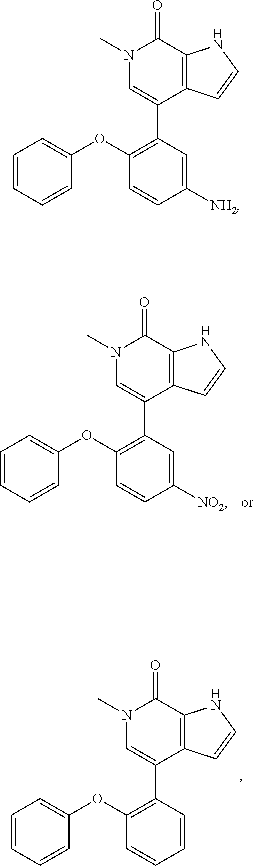 [SMILES: Cn1cc(-c2cc(N)ccc2Oc2ccccc2)c2cc[nH]c2c1=O ; Cn1cc(-c2cc([N+](=O)[O-])ccc2Oc2ccccc2)c2cc[nH]c2c1=O ; Cn1cc(-c2ccccc2Oc2ccccc2)c2cc[nH]c2c1=O]
each of which is substituted by one occurrence of R^{II-2A}, wherein R^{II-2A} is a bond to L.

104. The compound of any one of claims 98-100, wherein the EPL is

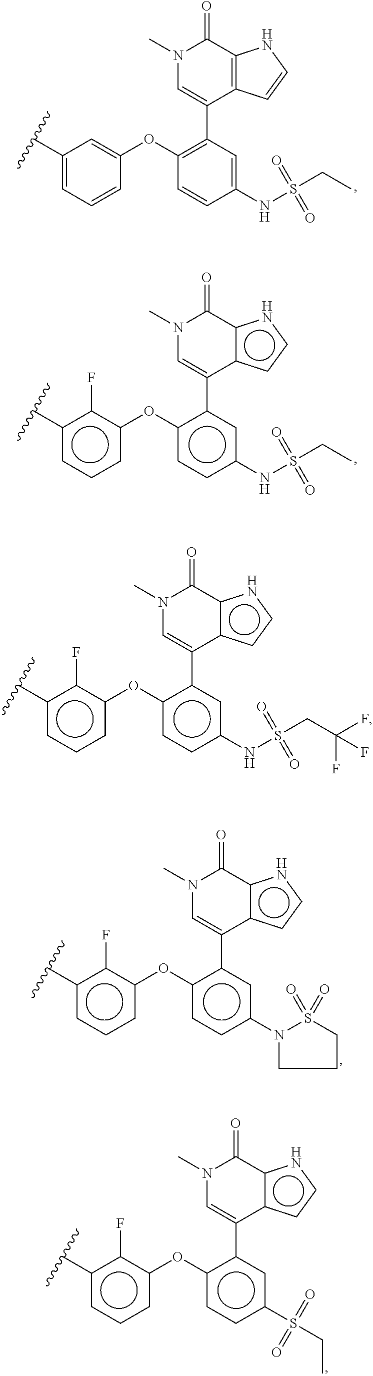

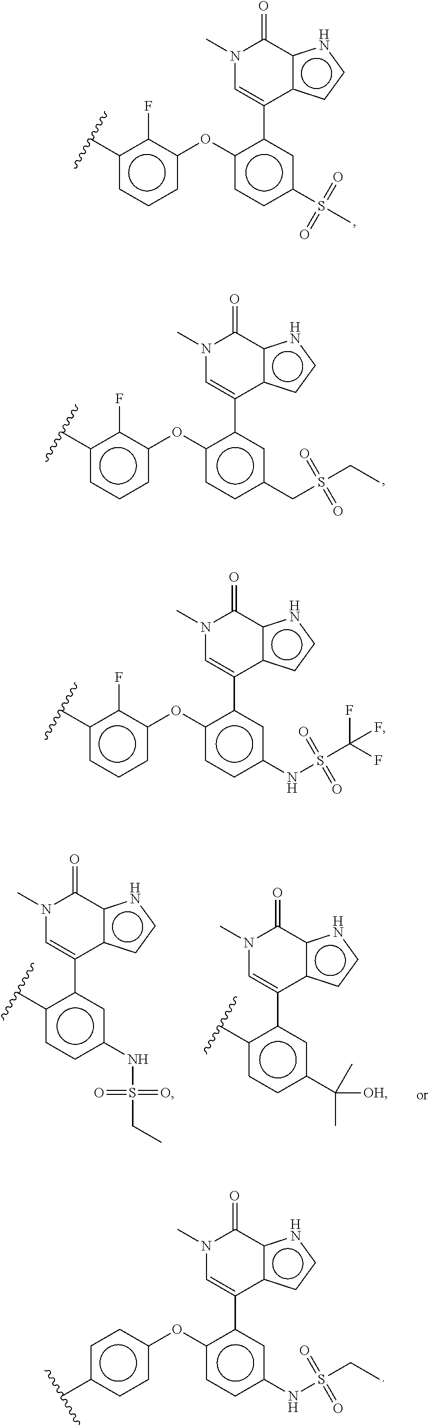

105. The compound of any one of claims 98-100, wherein the EPL is

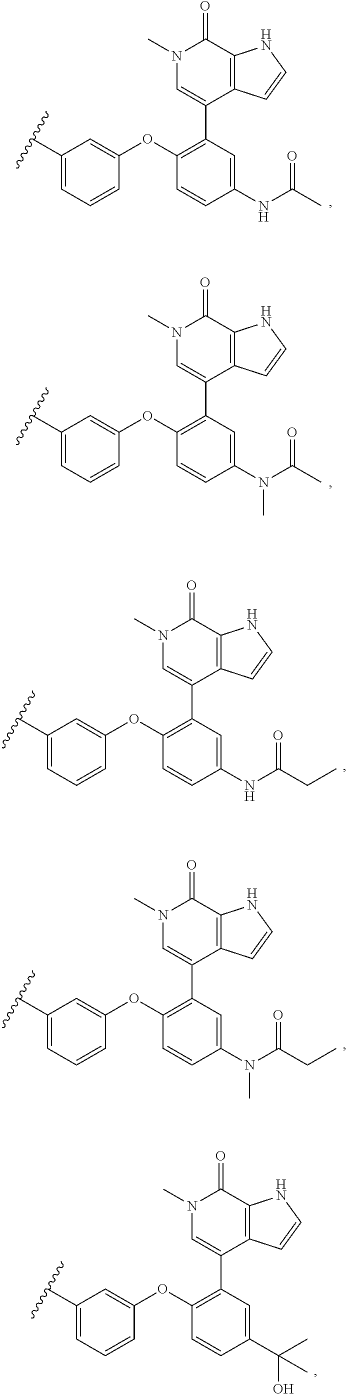 [SMILES: *c1cccc(Oc2ccc(NC(C)=O)cc2-c2cn(C)c(=O)c3[nH]ccc23)c1 ; *c1cccc(Oc2ccc(N(C)C(C)=O)cc2-c2cn(C)c(=O)c3[nH]ccc23)c1 ; *c1cccc(Oc2ccc(NC(=O)CC)cc2-c2cn(C)c(=O)c3[nH]ccc23)c1 ; *c1cccc(Oc2ccc(N(C)C(=O)CC)cc2-c2cn(C)c(=O)c3[nH]ccc23)c1 ; *c1cccc(Oc2ccc(C(C)(C)O)cc2-c2cn(C)c(=O)c3[nH]ccc23)c1]

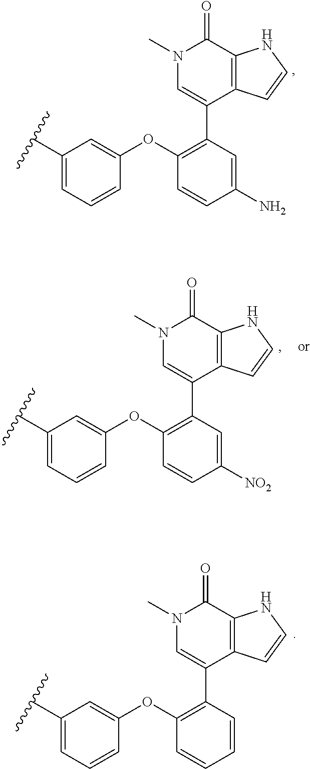 [SMILES: *c1cccc(Oc2ccc(N)cc2-c2cn(C)c(=O)c3[nH]ccc23)c1 ; *c1cccc(Oc2ccc([N+](=O)[O-])cc2-c2cn(C)c(=O)c3[nH]ccc23)c1 ; *c1cccc(Oc2ccccc2-c2cn(C)c(=O)c3[nH]ccc23)c1]

106. The compound of any one of claims 98-100, wherein the EPL is

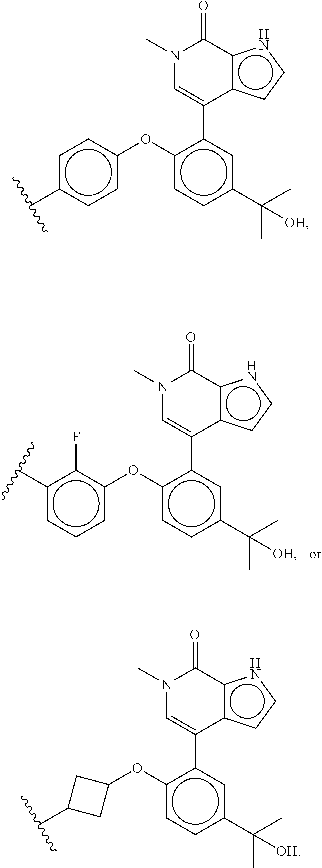

107. The compound of any one of claims 98-106, wherein L is a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, or optionally substituted 3-10 membered heterocyclyl containing 1, 2, 3, or 4 heteroatoms independently selected from nitrogen, oxygen, and sulfur.

108. The compound of any one of claims 98-106, wherein L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —N(H)C(O)—, —N(C₁₋₆ alkyl)C(O)—, —C(O)N(H)—, —C(O)N(C₁₋₆ alkyl)-, 3-10 membered carbocyclyl, or 3-10 membered heterocyclyl containing 1, 2, or 3 heteroatoms independently selected from nitrogen, oxygen, and sulfur.

109. The compound of any one of claims 98-106, wherein L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —N(H)C(O)—, —N(C₁₋₆ alkyl)C(O)—, —C(O)N(H)—, or —C(O)N(C₁₋₆ alkyl)-.

110. The compound of any one of claims 98-106, wherein L is one of the following: 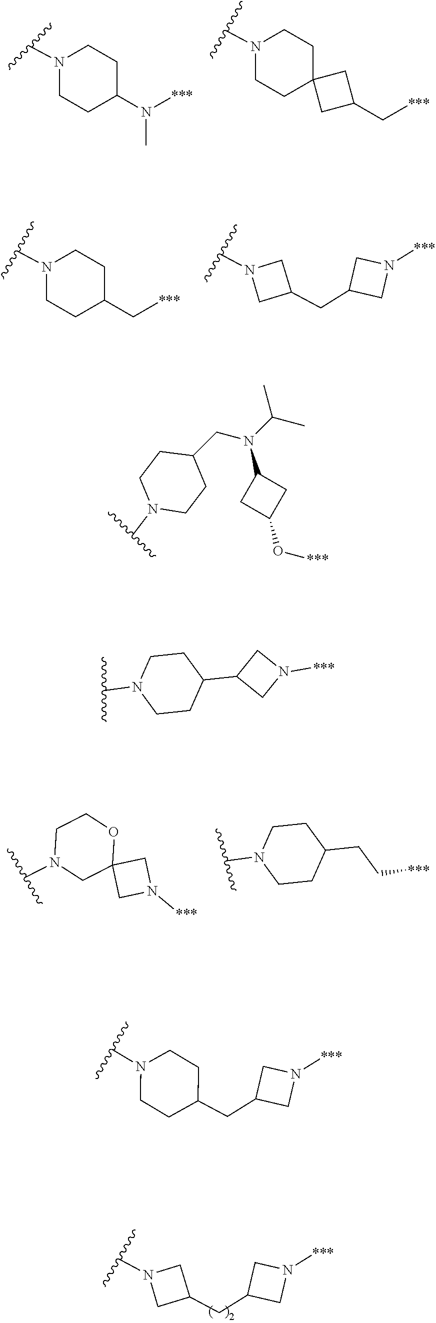
wherein *** is the point of attachment to A².

111. The compound of any one of claims 1-50 or 98-106, wherein L is -(3-7 membered, monocyclic, saturated heterocyclic ring containing 1 or 2 heteroatoms selected from nitrogen)-O—(C₁₋₆ alkylene)-(3-7 membered, monocyclic, saturated heterocyclic ring containing 1 or 2 heteroatoms selected from nitrogen)-(C₁₋₆ alkylene)-O—***, -(3-7 membered, monocyclic, saturated heterocyclic ring containing 1 or 2 heteroatoms selected from nitrogen)-(C₁₋₆ alkylene)-(C₁₋₆ alkylene)-(3-7 membered, monocyclic, saturated heterocyclic ring containing 1 or 2 heteroatoms selected from nitrogen)-(C₁₋₆ alkylene)-O—***, -(3-7 membered, monocyclic, saturated heterocyclic ring containing 1 or 2 heteroatoms selected from nitrogen)-(C₁₋₆ alkylene)-(C₀-6 alkylene)-(7-9 membered spirocyclic saturated heterocyclic ring containing 1 or 2 heteroatoms selected from nitrogen)-(C₀₋₆ alkylene)-***, -(7-12 membered spirocyclic saturated heterocyclic ring containing 1 or 2 heteroatoms selected from nitrogen)-(C₀₋₆ alkylene)-O—(C₀₋₆ alkylene)-***, -(7-12 membered spirocyclic saturated heterocyclic ring containing 1 or 2 heteroatoms selected from nitrogen)-(C₁₋₆ alkylene)-***, -(7-12 membered spirocyclic saturated heterocyclic ring containing 1 or 2 heteroatoms selected from nitrogen)-(C₃₋₆ cycloalkylene)-***, -(7-12 membered spirocyclic saturated heterocyclic ring containing 1 or 2 heteroatoms selected from nitrogen)-(3-7 membered, monocyclic, saturated heterocyclic ring containing 1 or 2 heteroatoms selected from nitrogen)-***, -(7-12 membered spirocyclic saturated heterocyclic ring containing 1 or 2 heteroatoms selected from nitrogen)-(C₂ alkynylene)-***, -(3-7 membered, monocyclic, saturated heterocyclic ring containing 1 or 2 heteroatoms selected from nitrogen)-O—(C₁₋₆ alkylene)-N(C₁₋₆ alkyl)C(O)—***, -(7-10 membered, fused bicyclic saturated heterocyclic ring containing 1 or 2 heteroatoms selected from nitrogen)-(C₁₋₆ alkylene)-***, -(7-10 membered, fused bicyclic saturated heterocyclic ring containing 1 or 2 heteroatoms selected from nitrogen)-(C₁₋₆ alkylene)-(3-7 membered, monocyclic, saturated heterocyclic ring containing 1 or 2 heteroatoms selected from nitrogen)-***, -(3-7 membered, monocyclic, saturated heterocyclic ring containing 1 or 2 heteroatoms selected from nitrogen)-(C₃₋₆ cycloalkylene)-N(C₁₋₆ alkyl)-(C₀₋₆ alkylene)-***, -(3-7 membered, monocyclic, saturated heterocyclic ring containing 1 or 2 heteroatoms selected from nitrogen)-N(C₁₋₆ alkyl)-(C₁₋₆ alkylene)-N(H)—(C₀₋₆ alkylene)-***, -(3-7 membered, monocyclic, saturated heterocyclic ring containing 1 or 2 heteroatoms selected from nitrogen)-N(C₁₋₆ alkyl)-(C₁₋₆ alkylene)-N(C₁₋₆ alkylene)-(C₀₋₆ alkylene)-***, -(3-7 membered, monocyclic, saturated heterocyclic ring containing 1 or 2 heteroatoms selected from nitrogen)-(C₁₋₆ alkylene)-N(C₁₋₆ alkyl)-(C₃₋₆ cycloalkylene)-O—(C₀₋₆ alkylene)-***. -(3-7 membered, monocyclic, saturated heterocyclic ring containing 1 or 2 heteroatoms selected from nitrogen)-N(C₃₋₆ cycloalkyl)-(3-7 membered, monocyclic, saturated heterocyclic ring containing 1 or 2 heteroatoms selected from nitrogen)-***, -(5-9 membered, bridged bicyclic, saturated heterocyclic ring containing 1 or 2 heteroatoms selected from nitrogen)-(C₁₋₆ alkylene)-(3-7 membered, monocyclic, saturated heterocyclic ring containing 1 or 2 heteroatoms selected from nitrogen)-***, -(3-7 membered monocyclic saturated heterocyclic ring containing 1 or 2 heteroatoms selected from nitrogen)-O-(3-7 membered monocyclic saturated heterocyclic ring containing 1 or 2 heteroatoms selected from nitrogen)-(C₁₋₆alkylene)-O—***, -(7-10 membered bridged bicyclic saturated heterocyclic ring containing 1 or 2 heteroatoms selected from nitrogen)-(C₁₋₆ alkylene)-(3-7 membered monocyclic saturated heterocyclic ring containing 1 or 2 heteroatoms selected from nitrogen)-(C₀₋₆ alkylene)-***, -(3-7 membered monocyclic saturated heterocyclic ring containing 1 or 2 heteroatoms selected from nitrogen)-(C₀₋₆ alkylene)-(7-11 membered spirocyclic saturated heterocyclic ring containing 1 or 2 heteroatoms selected from nitrogen)-O—(C₀₋₆ alkylene)-***. -(6-10 membered bicyclic saturated heterocyclic ring containing 1 or 2 heteroatoms selected from nitrogen)-(C₁₋₆ alkylene)-***, -(4-6 membered monocyclic heterocyclyl containing 1 or 2 heteroatoms selected from nitrogen)-(C₀₋₆ alklene)-O—(5-6 membered heteroaryl containing 1 or 2 heteroatoms selected from nitrogen)-O—***, -(4-6 membered monocyclic heterocyclyl containing 1 or 2 heteroatoms selected from nitrogen)-(C₀₋₆ alkylene)-(4-6 membered monocyclic heterocyclyl containing 1 or 2 heteroatoms selected from nitrogen)-O—***, or -(4-6 membered monocyclic heterocyclyl containing 1 or 2 heteroatoms selected from nitrogen)-(C₃₋₆ cycloalkylene)-(C₀₋₆ alkylene)-***, wherein *** is the point of attachment to A².

112. A compound in Table 1, or a pharmaceutically acceptable salt thereof.

113. The compound of claim 111, wherein the compound is selected from compounds I-1 through 1-103 in Table 1, or a pharmaceutically acceptable salt thereof.

114. A pharmaceutical composition comprising a compound of any one of claims 1-113 and a pharmaceutically acceptable carrier.

115. A method of treating cancer, comprising administering to a patient in need thereof a therapeutically effective amount of a compound of any one of claims 1-113 to treat the cancer.

116. The method of claim 115, wherein the cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct cancer, gallbladder cancer, liver cancer, pancreatic cancer, bladder cancer, urinary tract cancer, kidney cancer, eye cancer, thyroid cancer, lymphoma, or leukemia.

117. The method of claim 115, wherein the cancer is prostate cancer.

118. A method of causing death of a cancer cell, comprising contacting a cancer cell with an effective amount of a compound of any one of claims 1-113 to cause death of the cancer cell.

119. The method of claim 118, wherein the cancer cell is selected from an ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct cancer, gallbladder cancer, liver cancer, pancreatic cancer, bladder cancer, urinary tract cancer, kidney cancer, eye cancer, thyroid cancer, lymphoma, or leukemia cell.

120. The method of claim 119, wherein the cancer cell is a prostate cancer cell.
